(19) United States
(12) Patent Application Publication
(10) Pub. No.: US 2026/0158156 A1
(43) Pub. Date: Jun. 11, 2026

(54) SPECIFIC CONJUGATION OF AN ANTIBODY

(71) Applicant: HANGZHOU DAC BIOTECH CO., LTD., Hangzhou, Zhejiang (CN)

(72) Inventors: Robert ZHAO, Lexington, MA (US); Qingliang YANG, Hangzhou, Zhejiang (CN); Xiaolei LIU, Hangzhou, Zhejiang (CN); Lingli ZHANG, Hangzhou, Zhejiang (CN); Yuanyuan HUANG, Hangzhou, Zhejiang (CN); Wenjun LI, Hangzhou, Zhejiang (CN); Hangbo YE, Hangzhou, Zhejiang (CN); Juan WANG, Hangzhou, Zhejiang (CN); Huihui GUO, Hangzhou, Zhejiang (CN); You ZHOU, Hangzhou, Zhejiang (CN)

(73) Assignee: HANGZHOU DAC BIOTECH CO., LTD., Hangzhou, Zhejiang (CN)

(21) Appl. No.: 18/706,734

(22) PCT Filed: Nov. 3, 2021
(86) PCT No.: PCT/CN2021/128453

§ 371 Date: May 1, 2024

(51) Int. Cl. A61K 47/68; A61K 47/18; A61K 47/26; A61P 35/00; C07K 16/28; C07K 16/30

(52) U.S. Cl. CPC A61K 47/68037; A61K 47/183; A61K 47/26; A61K 47/68031; A61K 47/68033; A61K 47/68035; A61K 47/6809; A61K 47/6849; A61K 47/6851; A61K 47/6889; A61P 35/00; C07K 16/2863; C07K 16/2878; C07K 16/30

(57) ABSTRACT

Provided herein is a process for preparing a homogeneous conjugate of an antibody or antibody-like protein via linkage of cysteine sites between heavy-light chains in the IgG antibody or antibody-like protein. It also relates to method of making the conjugates in a specific manner including either generation of specific thiols of an antibody or antibody-like protein agent, followed by reaction with drug/linker complexes, or generation of specific thiols of an antibody or antibody-like protein agent and conjugation of a synthetic linker-drug assembly with the thiols simultaneously in one pot reaction, to provide conjugates with over 75%, in most cases more than 80% of payloads linked at the specific cysteine sites between heavy-light chains of the IgG antibody or antibody-like protein. It also relates to methods of using the homogeneous conjugate in targeted prophylaxis or treatment of cancer, infection and immunological disorders.

### FIELD OF THE INVENTION

The present invention relates to a process for preparing a homogeneous conjugate of an antibody or antibody-like protein molecule/agent via linkage of certain sulphurs of cysteine sites in the antibody. The present invention also relates to methods of making the conjugates in a specific manner comprising either generation of specific thiols of an antibody or antibody-like protein agent, followed by reaction with drug/linker complexes, or generation of specific thiols of an antibody or antibody-like protein agent and conjugation of a synthetic linker-drug assembly to the protein molecule simultaneously in one pot reaction. It also relates to methods of using the homogeneous conjugate in targeted prophylaxis or treatment of cancer, infection and immunological disorders.

### BACKGROUND OF THE INVENTION

Nowadays, the pace of ADC development is accelerating, with and the number of the clinical trials having more than tripled over the past 5 years and 7 of 12 marketed ADCs were approved by US FDA in last 2 years, underscoring the enthusiasm for this transformative approach to cancer treatment. The conjugation of payload and antibody via a linker is a critical aspect that defines ADC quality, safety, efficacy and the overall success (M. Acchione, H. Kwon, et al, 2012, mAbs 4:3, 362-372; M. J. Birrer, K. N. Moore, et al, 2019, J. National Cancer Inst., 111(6), 538-549). A report published by US FDA indicated that for most ADCs currently in clinical development, dose-limiting toxicities (DLT) appear to be unrelated to the targeted antigen, but driven by the payload/linker complexes (H. Saber and J. K. Leighton, Regulatory Toxicology and Pharmacology 71 (2015) 444-452); It also known that ADC linkers and the conjugation sites play critical roles in an ADCs' stability during preparation and storage, as well extent of systemic toxicity in the blood circulation in vivo (J. R. McCombs and S. C. Owen, 2015, AAPS Journal 17, 339-351).

Thus research and development into ADC chemistry and design are now expanding the scopes of the linker-payload compartments and conjugate chemistry to address the challenge of dose-limiting toxicities (DLT) of ADCs toward target diseases (Lambert, J. M. 2016, Ther. Deliv. 7, 279-82; Zhao, R. Y. et al, 2011, J. Med. Chem. 54, 3606-23). Nowadays, one of the extensive R&D effort for broader therapeutic windows (TW) of ADCs is to establish novel reliable methods for site-specific ADC conjugation, which seem to have longer circulation half-life, higher efficacy, potentially decreased off-target toxicity, and a narrow range of in vivo pharmacokinetic (PK) properties of ADCs as well as better batch-to-batch consistency in ADC production (Hussain, A. F., et al, Pharmaceuticals (Basel), 2021, 14(4), 343; Sadiki, A., et al, Antib Ther. 2020, 3(4), 271-284; Wolska-Washer, A.; Robak, T., Drug Saf, 2019, 42(2), 295-314; Tsuchikama, K., An, Z., Protein Cell. 2018, 9(1), 33-46; Thomas, A. et al, Lancet Oncol. 2016, 17(6), e254-e262; Strop, P., et al 2013 Chem. Biol. 20, 161-67; Wakankar, A. mAbs, 2011, 3, 161-172; Zhao, R. Y. et al, 2011, J. Med. Chem. 54, 3606-23).

There are several approaches developed in recent years for the site selective ADC preparation (Panowski, S, 2014, mAbs 6, 34). They include incorporation of unpaired cysteines, e.g. engineered reactive cysteine residues, called THIOMAB from Genentech (Junutula, J. R., et al 2010 Clin. Cancer Res. 16, 4769; Junutula, J. R., et al 2008 Nat Biotechnol. 26, 925-32; U.S. Pat. Nos. 8,309,300; 7,855,275; 7,521,541; 7,723,485, WO2008/141044), genetically introduced glutamine tag with Streptoverticillium mobaraense transglutaminase (mTG) (Strop, P., Bioconjugate Chem., 2014, 25, 855-862; Strop, P., et al., 2013, Chem. Biol. 20, 161-167; U.S. Pat. No. 8,871,908 for Rinat-Pfizer) or with Microbial transglutaminase (MTGase) (Dennler, P., et al, 2014, Bioconjug. Chem. 25, 569-578. US pat appl 20130189287 for Innate Pharma; U.S. Pat. No. 7,893,019 for Bio-Ker S.r.l. (IT)), incorporation of thiolfucose (Okeley, N. M., et al 2013 Bioconjugate Chem. 24, 1650), incorporation of unnatural amino acids through mutagenesis (Axup, J. Y., et al., 2012, Proc. Natl. Acad. Sci. 109, 16101-16106; Zimmerman, E. S., et al., 2014, Bioconjug. Chem. 25, 351-361; Wu, P., et al, 2009 Proc. Natl. Acad. Sci. 106, 3000-5; Rabuka, D., et al, 2012 Nat. Protoc. 7, 1052-67; U.S. Pat. No. 8,778,631 and US Pat Appl. 20100184135, WO2010/081110 for Sutro Biopharma; WO2006/069246, 2007/059312, U.S. Pat. Nos. 7,332,571, 7,696,312, and 7,638,299 for Ambrx; WO2007/130453, U.S. Pat. Nos. 7,632,492 and 7,829,659 for Allozyne), incorporation of selenocysteine into antibodies (Hofer, T., et al 2009, Biochemistry 48, 12047-12057; U.S. Pat. No. 8,916,159 for US National Cancer Institute), Conversion of cysteines located in the CXPXR consensus sequence to formylglycine (FGly) with formylglycine generating enzyme (FGE) (Drake, P. M., et al., 2014, Bioconjug. Chem. 25, 1331-1341; Carrico, I. S. et al U.S. Pat. Nos. 7,985,783; 8,097,701; 8,349,910, and US Pat Appl 20140141025, 20100210543 for Redwood Bioscience); via glycoengineeringly introduction of sialic acid with the use of galactosyl- and sialytransferases (Zhou, Q., et al 2014, Bioconjug.Chem., 25, 510-520, US Pat Appl 20140294867for Sanofi-Genzyme); the incorporation of a cyclopropene derivative of lysine, followed by Diels-Alder cycloaddition with tetrazine derivatives to yield 1, 4-dihydropyridazines in conjugation (Oller-Salvia, B. et al, 2018 Angew Chem Int Ed Engl, 57, 2831-2834). However, the above methods are required antibody-engineering processes and reoptimization of cell culture conditions, often suffer from low protein expression, low conjugation yields, or are limited to specific conjugation sites. Therefore, some simple homogeneous conjugation methods were practically studied through rebridging the reduced inter chain disulfide bonds of a native antibody, such as, using bromo or dibromo-maleimides, called next generation maleimides (NGMs) (Schumacher, F. F., et al 2014, Org. Biomol. Chem. 12, 7261-69; UCL Cancer Institute), or using dibromopyri-dazinediones (A. Maruani, et al, Nat. Commun., 2015, 6, 6645; M. T. Lee, et al, Chem. Sci., 2017, 8, 2056), or applying bis-alkylating reagents via a three-carbon bridge (Badescu, G., et al., 2014, Bioconjug. Chem. 25, 1124-36; WO2013/190272, WO2014/064424 for PolyTherics Ltd), or via arylenedipropiolonitrile (ADPN) molecule (Koniev, O., et al, 2018 MedChemComm. 2018, 9, 827-830). Recently, Coumans et al used 2-(diphenylphosphino)-benzenesulfonic acid (diPPBS) as the reducing agent that only reduced the engineered cysteines without harming the interchain disulfides in an antibody for preparation of ADCs (R. G. E. Coumans, et al, Bioconjugate Chem. 2020, 31, 2136-2146). Wuxi Biologics Co., applied Zn²⁺ ions (ZnCl₂ salt in a buffer) for selected control the cystine reduction in an antibody, followed by reaction with a payload containing maleimide linker to improve homogeneity in production of ADCs (PCT/CN2020/075162). However, most of antibodies are stored in a phosphate based buffer, e. g. called PBS buffer, Zn cation swiftly precipitate in a phosphate buffer to form zinc phosphate, since the solubility constant of zinc phosphate is 9.1×10⁻³³ at neutral or base pH conditions (Martin, R. Bruce. “Solubility and Solubility Products (about J. Chem. Educ. 1998, 75, 1179-1181 and J. Chem. Educ. 1998, 75, 1182-1185) “J. Chem. Educ. 2000, 77, 1558; Dupuis, V., et al, 1992, Biomaterials, 13 (7), 467-470; https://www.chm.uri.edu/weuler/chm112/refmater/KspTable.html). Therefore, when using ZnCl₂ in the reduction of an antibody, the amount of phosphate anions and the pH of the buffers have to be precisely controlled to avoid formation of the precipitate of zinc phosphate.

We have disclosed several conjugation methods of rebridging a pair of thiols of the reduced inter chain disulfide bonds of a native antibody, such as using bromo maleimide and dibromomaleimide linkers (WO2014/009774), 2, 3-disubstituted succinic/2-monosubstituted/2, 3-disubstituted fumaric or maleic linkers (WO2015/155753, WO2016/596228), acetylenedicarboxylic linkers (WO2015/151080, WO2016/596228), hydrazine linkers (WO2015/151081) and acryloyl or propiolyl linkers (WO2018/086139). In this patent application, we extend the scopes of our earlier patent application in production of more homogeneous conjugates via selective control reduction of interchains of disulfide bonds of an antibody follow by or simultaneous conjugation under coordinative help by a zinc amino complex/chelate. Zinc amino complexes have more advantages over ZnCl₂ in coordination of reduction of disulfide bond in an antibody. First, zinc amino complexes are much bulkier than ZnCl₂ and can be more 3-D space selectively to be inserted in certain positions (e. g. more specifically in the positions of the disulfide bonds between heavy-light chains of an IgG1 antibody); Second, zinc amino complexes are more stable in a water based solution, for instance, the stability constant of zinc ammonia complex ion is 2.9×10⁹ (https://chempedia.info/info/stability_constants/), which in turn, slow the precipitation in a neutral pH phosphate buffer. More importantly, by uses of zinc amino complexes to stoichiometrically control reduction of the disulfur bonds between the light chain and heavy chain of an IgG antibody without hurting the disulfide bonds between the heavy chains (in the hinge region) of the antibody, a bis-alkylation of the two geometrically adjacent thiols can be well specifically achieved since our former data (WO2016/059622) and the other research results (S. Shao, et al, Bioorg Med Chem Lett. 2018, 28, 1363) demonstrated that the bis-alkylation of two adjacent thiols was favored in intra-heavy chain fashion in hinge region rather than conjugated between interchain disulfide bonds. In a word, the conjugation strategy of this invention has robust manufacturability to yield highly homogeneous ADCs without antibody engineering and can successfully tackle an important shortcoming in current ADC preparation methods. This conjugation strategy can be applied directly to other antibody likes of proteins. The resulting homogeneous ADCs demonstrate improved pharmacokinetics, superior efficacy, and reduced toxicity in vivo compared to analogous conventional heterogeneous ADCs.

### SUMMARY OF THE INVENTION

The present invention provides conjugation process with improved homogeneity of an antibody conjugate, or antibody-like protein conjugate, in particular, an antibody-drug conjugate (ADC), wherein over 75% of payloads (drugs) are specifically conjugated to the disulfide bond sites between heavy-light chains of an antibody.

The homogenous conjugation process comprises the following three key steps:
(a) incubating an antibody-like protein, in particular, an IgG antibody in the presence of an effective amount of transition metal cation-amino chelate/complex (M(NR₁R₂R₃)ₘ₁^{m2+}) and a reductant (e.g. Tris(2-carboxyethyl)phosphine (TCEP)) in a buffer system (e. g. PBS, Mes, Bis-Tris, Bis-Tris Propane, Pipes, Aces, Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetates, Histidine, Citrates, MES, or Borates, etc.) to selectively reduce interchain disulfide bonds within the antibody, or antibody-like protein to generate thiols;
(b). introducing an effective amount of linker or payload/linker complex/assembly bearing thiol reactive groups (e.g., a drug containing maleimide terminal) to react with the thiol groups resulted from step (a); and
(c). adding an effective amount of oxidant (e. g. dehydroascorbic acid (DHAA)) to re-oxidize unreacted thiol groups and then purifying the resulted conjugates;
(d). the step (c) can be replaced by: adding an effective amount of cystine or relative disulfide compound to quench the unreacted reductant, while generating cysteine from the reduction of the cystine to quench the excessive conjugation linker or linker/payload complex containing thiol reactive groups (e. g. maleimide).

The transition metal cation-amino chelate/complex, M(NR₁R₂R₃)ₘ₁^{m2+}, wherein M is selected from, but not limited to, Zn²⁺, Cu²⁺, Fe²⁺, Cd²⁺, Ni²⁺, Cr²⁺, Cr³⁺, Ti²⁺, Ti³⁺, Co²⁺, Mn²⁺, Mn³⁺, Ag⁺, Hg²⁺; wherein R₁, R₂ and R₃ are independently selected from C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; ml is selected from 1, 2, 3, 4, 5, 6, 7 or 8; m2 is selected from 1, 2, 3, 4, 5, or 6. Preferably M is Zn; ml is 1, 2, 3 or 4; and m2 is 1, 2, 3, or 4.

In addition, (NR₁R₂R₃)ₘ₁ can be form a dimer, trimer, tetramer, pentamer, or hexamer wherein these polymers are covalently linked among N, R₁, R₂ and R₃; and N, R₁, R₂ or R₃ themselves can form heterocyclic, carbocyclic, diheterocyclic, or dicarbocyclic rings.

The transition metal cation-amino chelate/complex, M (NR₁R₂R₃)ₘ₁^{m2+}, used in step (a) is 0.01 mM-1.0 mM in concentration, or 0.5˜20 equivalents in moles of the protein, and it can be added to the reaction solution with a water-soluble organic solvent, selected from, ethanol, methanol, propanol, propandiol, DMA, DMF, DMSO, THF, CH₃CN.

The reductant is an organic phosphine, preferably selected from Tris(2-carboxyethyl)phosphine (TECP) or Tris(hydroxypropyl)phosphine and its use in the reaction solution is 0.02 mM-1.0 mM in concentration, or 1.0-20 equivalents in moles of the protein. The oxidant to be added in step (c) may be DHAA, Fe³⁺, I₂, Cu²⁺, Mn³⁺, MnO₂, or mixture of Fe³⁺/I⁻. The oxidant used in the reaction solution is 0.02 mM-1.0 mM in concentration, or 1-100 equivalents in moles of the protein. The optimum pH in the conjugation reaction is typically between about 5.0 to 8.0, and preferably, about 5.5 to 7.5. The optimum temperature in the conjugation reaction is typically between about −5 to about 40° C. and preferably, about 0 to 37° C.; more preferably about 2 to 8° C. The optimum time of the conjugation reaction is typically between about 15 min to about 48 hours and preferably, about 30 min to overnight (10˜16 h). The optimal reaction conditions (e. g. pH, temperature, buffer, concentrations of the reactants) of course are depended upon specifically an antibody-like protein, a payload/linker complex, a reductant and/or M(NR₁R₂R₃)ₘ₁^{m2+} used.

The antibody or antibody-like protein in the conjugation process can be any types of antibodies or proteins as long as they have two or more disulfide bonds in the protein for differentiation of reduction. And the payload/linker complex may be any types or formats as long as it has a thiol reactive group.

In a word, the ADCs prepared by the process of the present application have more than 80% of payloads conjugated in the Fab region of an antibody, in contrast to the conventional process wherein around 40% of the payloads are in the Fab region of an antibody and about 70% of the payloads are in the Fab region of an antibody using the process of WO2020164561. The advantages of the application along with the improved conjugation process for homogeneity of ADCs will become more apparent from the following detailed description of several embodiments, experimentals and figures.

### BRIEF DESCRIPTION OF THE DRAWINGS

FIG. 1. The proposed mechanism that zinc amino complexes coordinate the reduction of the disulfide bonds in an antibody.

FIG. 2. Middle-level characterization of ADC after N-deglycosylation and reduction. (a) rpHPLC chromatogram of ADC fragments obtained after deglycosylation and DTT reduction. Light chains (LC) with zero or one drug molecule attached (L0 and L1), and heavy chains with zero, one, two, or three drug molecules attached (H0, H1, H2 and H3). (b) Table summarizing masses and proportions of the different ADC fragments and the average DAR measured from peak areas. The results demonstrated the payload was conjugated mainly at the sites between the light-heavy chains.

FIG. 3. MS and MS/MS spectra of drug/linker (C-408b)-loaded peptides. (a) [GEC]+1 payload, (b) [SCDK]+1 payload, demonstrated the payload was conjugated mainly at the sites between light-heavy chain.

FIG. 4. The Percentage of Drug Loaded Peptides which were generated with hydrolases from the BCMA conjugate C-408b and analysized with UPLC-MS. (a). Light chain (LC) Peptide [GEC] with zero or one drug molecule attached (D0 and D1), (b). Heavy chain (HC) Peptide [SCDK] at the arm with zero or one drug molecule attached (D0 and D1), (C) HC Peptide [THTCPPCPAPELLXXXXXXXXXXX XX] at the hinge with zero, one or two drug molecule attached (D0, D1 and D2). (X here is an amino acid that will be disclosed in a separated patent application). The results demonstrated the payloads were conjugated mainly (over 85%) at the cysteine sites between the light-heavy chains of the antibody.

FIG. 5. HIC-HPLC analysis of BCMA antibody conjugate of C-406, which was prepared with regular conjugation process (without coordination of a zinc amino complex, 2.2˜4.0 eq of TCEP, pH 7.0˜7.2). (5a): RT, 6.0 eq of compound 406, 4 h conjugation, DAR=4.0, D4=41.96%; (5b): 4±2° C., 6.0 eq of compound 406, 5 h conjugation, DAR=4.2, D4=47.16%; (5c): 4±2° C., 6.5 eq of compound 406, 5 h conjugation, DAR=5.1, D4=40.03%.

FIG. 6. HIC-HPLC analysis of BCMA antibody conjugate of C-406, which was prepared with coordination of 2.0˜2.4 eq of zinc chloride, pH=7.2, 4±2° C. for 15±2 h. (6a): 2.0 eq of ZnCl₂, 4.0 eq of TCEP, 6.0 eq of compound 406, DAR=4.2, D4=68.15%; (6b): 2.2 eq of ZnCl₂, 4.0 eq of TCEP, 6.5 eq of compound 406, DAR=4.7, D4=68.39%; (6c): 2.4 eq of ZnCl₂, 3.5 eq of TCEP, 6.5 eq of compound 406, DAR=4.6, D4=62.53%; (6d): 2.4 eq of ZnCl₂, 4.0 eq of TCEP, 6.5 eq of compound 406, DAR=5.1, D4=65.16%.

FIG. 7. HIC-HPLC analysis of BCMA antibody conjugate of C-406, which was prepared with coordination of 2.4 eq of Z-11, 4.0 eq of TCEP, 6.5 eq of compound 406, pH=7.2, 4±2° C. for 5 h, DAR=4.6, D4=79.810%.

FIG. 8. HIC-HPLC analysis of EGFR antibody conjugate of C-038, which was prepared with coordination of 2.4 eq of Z-28, 3.6 eq of TCEP, 6.0 eq of compound 038, pH=7.2, 4±2° C. for 5 h, DAR=4.2, D4=79.68%.

FIG. 9. HIC-HPLC analysis of EGFR antibody conjugate of C-111, which was prepared with coordination of 2.4 eq of Z-28, 3.4 eq of TCEP, 6.0 eq of compound 111, pH=7.2, 4 2° C. for 5 h, DAR=4.4, D4=80.39%.

FIG. 10. HIC-HPLC analysis of EGFR antibody conjugate of C-226, which was prepared with coordination of 2.4 eq of Z-28, 3.4 eq of TCEP, 6.0 eq of compound 226, pH=7.2, 4±2° C. for 5 h, DAR=4.4, D4=77.94%;

FIG. 11. HIC-HPLC analysis of EGFR antibody conjugate of C-227, which was prepared with coordination of 2.4 eq of Z-28, 3.4 eq of TCEP, 6.0 eq of compound 038, pH=7.2, 4±2° C. for 5 h, DAR=4.0, D4=78.73%.

FIG. 12. HIC-HPLC analysis of BCMA antibody conjugate of C-325, which was prepared with coordination of 2.4 eq of Z-28, 3.6 eq of TCEP, 6.0 eq of compound 325, pH=7.2, 4±2° C. for 5 h, DAR=4.1, D4=80.11%.

FIG. 13. HIC-HPLC analysis of Trop2 antibody conjugate of C-334, which was prepared with coordination of 2.4 eq of Z-28, 3.6 eq of TCEP, 6.0 eq of compound 334, pH=7.2, 4±2° C. for 5 h, DAR=4.2, D4=81.15%.

FIG. 14. HIC-HPLC analysis of Her2 antibody conjugate of C-334, which was prepared with coordination of 2.4 eq of Z-28, 3.6 eq of TCEP, 6.0 eq of compound 334, pH=7.2, 4±2° C. for 5 h, DAR=4.4, D4=80.82%.

FIG. 15. HIC-HPLC analysis of CD33 antibody conjugate of C-334, which was prepared with coordination of 2.4 eq of Z-28, 3.6 eq of TCEP, 6.0 eq of compound 334, pH=7.2, 4±2° C. for 5 h, DAR=4.4, D4=79.93%.

FIG. 16. HIC-HPLC analysis of EGFR antibody conjugate of C-379, which was prepared with coordination of 2.4 eq of Z-28, 3.6 eq of TCEP, 6.0 eq of compound 379, pH=7.2, 4±2° C. for 5 h, DAR=4.4, D4=79.02%.

FIG. 17. HIC-HPLC analysis of EGFR antibody conjugate of C-385, which was prepared with coordination of 2.2 eq of Z-21, 3.6 eq of TCEP, 6.0 eq of compound 385, pH=7.2, 4±2° C. for 5 h, DAR=3.8, D4=69.54%.

FIG. 18. HIC-HPLC analysis of EGFR antibody conjugate of C-387, which was prepared with coordination of 2.2 eq of Z-21, 3.6 eq of TCEP, 6.0 eq of compound 387, pH=7.2, 4±2° C. for 5 h, DAR=4.1, D4=77.25%.

FIG. 19. HIC-HPLC analysis of CD33 antibody conjugate of C-413d, which was prepared with coordination of 2.2 eq of Z-21, 3.6 eq of TCEP, 6.0 eq of compound 413d, pH=7.2, 4±2° C. for 5 h, DAR=4.1, D4=76.70%.

FIG. 20. HIC-HPLC analysis of EGFR antibody conjugate of C-422a, which was prepared with coordination of 2.2 eq of Z-21, 3.4 eq of TCEP, 6.0 eq of compound 422a, pH=7.2, 4±2° C. for 5 h, DAR=4.2, D4=76.58%.

FIG. 21. HIC-HPLC analysis of EGFR antibody conjugate of C-431a, which was prepared with coordination of 2.2 eq of Z-21, 3.6 eq of TCEP, 6.0 eq of compound 431a, pH=7.2, 4±2° C. for 5 h, DAR=4.1, D4=77.97%.

FIG. 22. HIC-HPLC analysis of Her2 antibody conjugate of C-431a, which was prepared with coordination of 2.2 eq of Z-21, 3.6 eq of TCEP, 6.0 eq of compound 431a, pH=7.2, 4±2° C. for 5 h, DAR=4.2, D4=79.44%.

FIG. 23. HIC-HPLC analysis of Steap1 antibody conjugate of C-412c, which was prepared with coordination of 2.2 eq of Z-21, 3.6 eq of TCEP, 6.0 eq of compound 412c, pH=7.2, 4±2° C. for 5 h, DAR=4.4, D4=80.01%.

FIG. 24. HIC-HPLC analysis of Steap1 antibody conjugate of C-428c, which was prepared with coordination of 2.2 eq of Z-21, 3.6 eq of TCEP, 6.0 eq of compound 428c, pH=7.2, 4±2° C. for 5 h, DAR=4.4, D4=79.83%.

FIG. 25 shows the comparison of the anti-tumor effect of EGFR antibody conjugate of C-031, C-038, C-066, C-071, C-093, C-111, C-118, C-208, C-214, and C-216, prepared through the methods of this patent application (all D4 >75%, except C-066 and C-071 having D8 >75%), along with paclitaxel plus naked EGFR antibody, C-038 conjugate with regular conjugation method having D4=41%, and PBS buffer, using human lung adenocarcinoma HCC-827 cell model at dosing of 6 mg/kg, i.v., one injection. The figure indicates that all the 10 conjugates had better antitumor activity than paclitaxel plus naked EGFR antibody (which was given at dosing of 6 mg/kg of EGFR antibody and 10 mg/kg of paclitaxel i.v., 3×QW (weekly injection for 3 weeks)), and the conjugate C-038 prepared with the method of this invention had better in vivo activity than that prepared by the regular method.

FIG. 26 shows the comparison of the anti-tumor effect of Trop2 antibody conjugate of C-216, C-218, C-328, C-384, C-408b, C-412c, C-422a, C-425a, and C-431c, prepared through the methods of this invention (all of them were prepared by the invention having D4 >75% (78˜83%), DAR=4.2˜4.4), along with C-408b conjugate with regular conjugation method having D4=42%, DAR=4.2, and PBS buffer, using human gastric cancer NCI-N87 cell model at dosing of 6 mg/kg, i.v., one injection. The figure indicates that all the 9 conjugates had antitumor activity, and the conjugate C-408b prepared with the method of this invention had better in vivo activity than that prepared by the regular method.

FIG. 27 shows the comparison of the anti-tumor effect of BCMA antibody conjugate of C-227, C-403a, C-403b, C-408b, C-412e, C-412f, C-428c, and C-431a, prepared through the methods of this invention (all of them were prepared by the invention having D4 >75% (78˜83%), DAR=4.1˜4.4), along with C-408b conjugate with regular conjugation method having D4=47%, DAR=4.2, and PBS buffer, using human multiple myeloma NCI-H929 cell model at dosing of 6 mg/kg, i.v., one injection. The figure indicates that all the 9 conjugates had antitumor activity, and the conjugate C-408b prepared with the method of this invention had better in vivo activity than that prepared by the regular method.

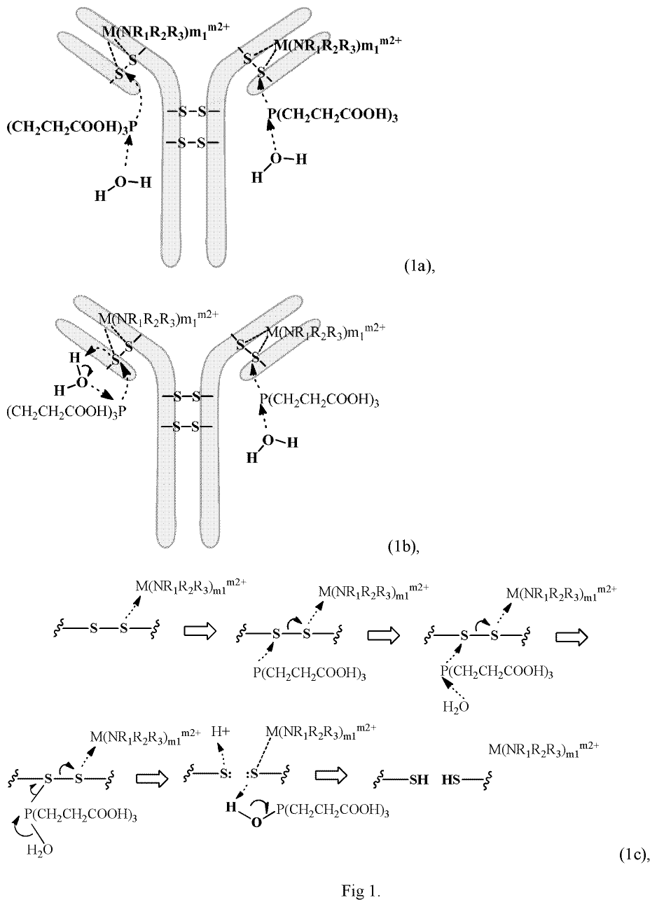

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having Ci-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2, 2-dimethylbutyl, 2, 3-dimethylbutyl, 2, 2-dimethylpentyl, 2, 3-dimethylpentyl, 3, 3-dimethylpentyl, 2, 3, 4-trimethylpentyl, 3-methyl-hexyl, 2, 2-dimethylhexyl, 2, 4-dimethylhexyl, 2, 5-dimethylhexyl, 3, 5-dimethylhexyl, 2, 4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4, 5], [5, 5], [5, 6] or [6, 6] system, or 9 or 10 ring atoms arranged as a bicycle [5, 6] or [6, 6] system. Representative C₃-C₈ carbocycles include, but are not limited to, -cyclopropyl, -cyclobutyl, -cyclopentyl, -cyclopentadienyl, -cyclohexyl, -cyclohexenyl, -1, 3-cyclohexadienyl, -1, 4-cyclohexadienyl, -cycloheptyl, -1, 3-cycloheptadienyl, -1, 3, 5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1, 2-ethyl (—CH₂CH₂—), 1, 3-propyl (—CH₂CH₂CH₂—), 1, 4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1, 2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1, 2, 4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” include, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoro-methylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1, 1′-carbonyldiimidazole; DCC, dicyclohexylcarbodiimide; DCE, dichloroethane; DCM, dichloromethane; DIAD, diisopropylazodicarboxylate; DIBAL-H, diisobutyl-aluminium hydride; DIPEA, diisopropylethylamine; DEPC, diethyl phosphorocyanidate; DMA, N, N-dimethyl acetamide; DMAP, 4-(N, N-dimethylamino)pyridine; DMF, N, N-dimethylformamide; DMSO, dimethylsulfoxide; DTT, dithiothreitol; EDC, 1-(3-dimethylaminopropyl)-3-ethylcarbodiimide hydrochloride; ESI-MS, electrospray mass spectrometry; HATU, 0-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuc-cinimide; MMP, 4-methylmorpholine; PAB, p-aminobenzyl; PBS, phosphate-buffered saline (pH 7.0˜7.5); PEG, polyethylene glycol; SEC, size-exclusion chromatography; TCEP, tris(2-carboxyethyl)phosphine; TFA, trifluoroacetic acid; THF, tetrahydrofuran; Val, valine.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3, 4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Val-Ala-Val, Lys-Lys, Ala-Asn-Val, Val-Leu-Lys, Cit-Cit, Val-Lys, Ala-Ala-Asn, Lys, Cit, Ser, and Glu.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, PA, 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

The abbreviations of biological buffers and their chemical names are listed below:
ACES (N-(2-Acetamido)-2-aminoethanesulfonic acid) is used to buffer at pH 6.1-7.5 (pKa=0.88)
ADA (N-(2-Acetamido)iminodiacetic acid, N-(Carbamoylmethyl)iminodiacetic acid) is useful buffer at pH 6.0-7.2 (pKa=6.65).
AMPD (2-amino-2-methyl-1, 3-propanediol)) is a useful buffer at pH 7.8-9.7.
AMPSO (N-(1, 1-Dimethyl-2-hydroxyethyl)-3-amino-2-hydroxypropanesulfonic acid).
BES (N, N-Bis(2-hydroxyethyl)-2-aminoethanesulfonic acid).
Bicine (N, N-Bis(2-hydroxyethyl)glycine], Bis(2-hydroxyethyl)amino-tris(hydroxymethyl) iethane) is used to buffer at pH 5.8-7.2 (pKa=8.35).
BisTris (Bis-(2-Hydroxyethyl)amino-tris(Hydroxymethyl)Methane).
BisTris propane (1, 3-Bis[tris(hydroxymethyl)methylamino]propane).
DIPSO (N, N-Bis(2-hydroxyethyl)-3-amino-2-hydroxypropanesulfonic acid) is used to buffer H 7.0-8.2.
Gly-Gly (Diglycine; Glycyl-glycine) is used to buffer at pH 7.5-8.9 (pKa=8.30).
HEBPS (N-(2-Hydroxyethyl)piperazine-N′-(4-butanesulfonic acid)) is an homolog of HEPES ad EPPS with higher pKa (pKa=8.30), used to buffer at pH 7.6-9.0
HEPES (4-(2-hydroxyethyl)-1-piperazineethanesulfonic acid; 2-morpholinoethanesulfonic acid; 2-(4-morpholino)ethanesulphonic acid; 2-(N-morpholino)ethanesulfonic acid; morpholine-4-thanesulfonic acid hydrate) is widely used to buffer at pH 6.8-8.2; pKa at 20° C.: 7.45-7.65)
HEPPS or EPPS (3-[4-(2-Hydroxyethyl)-1-piperazinyl]propanesulfonic acid hydrate; 4-(2-Hydroxyethyl)piperazine-1-(2-hydroxypropanesulfonic acid) Hydrate) is used as a buffering agent at pH 7.3-8.7 (pKa=8.00/piperazine ring).
HEPPSO (4-(2-Hydroxyethyl)piperazine-1-(2-hydroxypropanesulfonic acid) hydrate).
MES (2-(N-morpholino)ethanesulfonic acid, monohydrate) is used as buffering agent at pH 5.2-7.1 (pKa:6.16).
MOBS (4-Morpholinebutanesulfonic acid; 3-(N-Morpholino)butanesulfonic acid hemisodium salt) is an homolog of MES and MOPS with higher pKa/ It is used to buffer solution at pH6.9-8.3 (pKa:7.6).
MOPS (4-Morpholinepropanesulfonic acid Sodium salt).
MOPSO (β-Hydroxy-4-morpholinepropanesulfonic acid, 3-Morpholino-2-hydroxypropanesulfonic acid).
PIPES (Piperazine-1, 4-bis(2-ethanesulfonic acid) is used to buffer at pH 6.1-7.5 (pKa=6.80).
POPSO (Piperazine-1, 4-bis(2-hydroxypropanesulfonic acid) dihydrate).
TAPS ([(2-Hydroxy-1, 1-bis(hydroxymethyl)ethyl)amino]-1-propanesulfonic acid).
TAPSO (2-Hydroxy-3-[tris(hydroxymethyl)methylamino]-1-propanesulfonic acid).
TES (2-[(2-Hydroxy-1, 1-bis(hydroxymethyl)ethyl)amino]ethanesulfonic acid).
Tricine (Piperazine-N, N′-Bis[2-Hydroxypropanesulfonic Acid)] is used to buffer at pH7.4-8.8 (pKa:8.16).

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150: 880-887 (1993); Clarkson et al., Nature 352: 624-628 (1991).

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. Preferably, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “monovalent antibody” comprises one antigen binding site per molecule (e.g., IgG or Fab). In some instances, a monovalent antibody can have more than one antigen binding sites, but the binding sites are from different antigens.

A “monospecific antibody” comprises two identical antigen binding sites per molecule (e.g. IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g. Fab).

A “bivalent antibody” comprises two antigen binding sites per molecule (e.g., IgG). In some instances, the two binding sites have the same antigen specificities. However, bivalent antibodies may be bispecific.

A “bispecific” or “dual-specific” is a hybrid antibody having two different antigen binding sites. The two antigen binding sites of a bispecific antibody bind to two different epitopes, which may reside on the same or different protein targets.

A “bifunctional” is antibody is an antibody having identical antigen binding sites (i.e., identical amino acid sequences) in the two arms but each binding site can recognize two different antigens.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

A “heterodimer”, “heterodimeric protein”, “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

The “hinge region”, “hinge sequence”, and variations thereof, as used herein, includes the meaning known in the art, which is illustrated in, for example, Janeway et al., ImmunoBiology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The “immunoglobulin-like hinge region”, “immunoglobulin-like hinge sequence,” and variations thereof, as used herein, refer to the hinge region and hinge sequence of an immunoglobulin-like or an antibody-like molecule (e.g., immunoadhesins). In some embodiments, the immunoglobulin-like hinge region can be from or derived from any IgGI, IgG2, IgG3, or IgG4 subtype, or from IgA, IgE, IgD or IgM, including chimeric forms thereof, e.g., a chimeric IgGl/2 hinge region.

The term “immune effector cell” or “effector cell” as used herein refers to a cell within the natural repertoire of cells in the human immune system which can be activated to affect the viability of a target cell. The viability of a target cell can include cell survival, proliferation, and/or ability to interact with other cells.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4): 924-40, 2007).

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, In111, Re186, Re188, Sm153, Bi212, P32, Pb212, Zr89, F18, and radioactive isotopes of Lu, e.g. Lu177); chemotherapeutic agents or drugs (e.g., tubulysin, maytansin, auristatin, DNA minor groove binders (such as PBD dimers), ducarmysin, topoisomerase inhibitor, RNA polymerase inhibitors, DNA alkylators, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed throughout the application.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR2)nO(CR2) n-, repeating units of alkyloxy (e.g. polyethylenoxy, PEG, polymethyleneoxy) and alkylamino (e.g. polyethyleneamino); and diacid ester and amides including succinate, succinamide, diglycolate, malonate, and caproamide. In various embodiments, linkers can comprise one or more amino acid residues, such as valine, phenylalanine, lysine, and homolysine.

The words “comprise”, “comprising”, “include”, “including” and “includes” when used in this specification and claims are intended to specify the presence of stated features, integers, components, or steps, but they do not preclude the presence or addition of one or more other features, integers, components, steps, or groups thereof. The novel conjugates disclosed herein use the bridge linkers. Examples of some suitable linkers and their synthesis are shown in the examples 1-468 below of the specification.

The Conjugation Process of the Present Invention As disclosed above, the key factor of the invention in the conjugation process is the transition metal cation-amino complex, M (NR₁R₂R₃)ₘ₁^{m2+}, which coordinate the selective reduction of certain chain of disulfide bonds of biomolecule. Preferably the certain inter-chain disulfide bonds in an antibody. Previous study by LC-MS indicated that inter chain disulfide bonds of IgG antibodies are more susceptible to reduction than intra chain disulfide bonds, and the disulfide bonds between the light chain and heavy chain were more susceptible than disulfide bonds between the two heavy chains. The upper disulfide bond of the two inter heavy chain disulfide bonds of IgG antibodies was more susceptible than the lower one. Furthermore, disulfide bonds in the CH2 domain were the most susceptible to reduction. Disulfide bonds in VL, CL, VH, and CH1 domains had similar and moderate susceptibility, while disulfide bonds in the CH3 domain were the least susceptible to reduction (Liu, H, et al Anal. Chem., 2010, 82, 5219-5226). The using of ZnCl₂ salt at low temperatures of 2˜8° C. in coordination the reduction of the disulfide bonds of a IgG antibody of the invention WO2020164561 made practically possible of the distinguishable reduction above. Here, the transition metal cation-amino complex, M (NR₁R₂R₃)ₘ₁^{m2+}, which is used in the conjugation process of the invention, is much bulky, not only can coordinate the disulfide reduction, but also stereoscopically hinders the reductant (such as TCEP) to access to disulfide bonds between the two heavy chains of an IgG antibody, thus results in much better selective reduction and following by conjugation with a drug/linker complex.

The transition metal cation-amino chelate/complex, M(NR₁R₂R₃)ₘ₁^{m2+}, wherein M is selected from, but not limited to, Zn²⁺, Cu²⁺, Fe²⁺, Cd²⁺, Ni²⁺, Cr²⁺, Cr³⁺, Ti²⁺, Ti³⁺, Co²⁺, Mn²⁺, Mn³⁺, Ag⁺, Hg²⁺; wherein R₁, R₂ and R₃ are independently selected from C₁-C₈ of alkyl; C₂-C₈ of heteroalkyl, alkylcycloalkyl, heterocycloalkyl; C₃-C₈ of aryl, Ar-alkyl, heteroaryl, heterocyclic, carbocyclic, cycloalkyl, heteroalkylcycloalkyl, alkylcarbonyl, heteroaryl; m1 is selected from 1, 2, 3, 4, 5, 6, 7, or 8; m₂is selected from 1, 2, 3, 4, 5, 6, or 7. In preference, M is selected from Zn2+, m₁ is selected from 1, 2, 3, or 4, and m₂ is 2; 3 or 4. mᵢ is more preferably 2.

In addition, (NR₁R₂R₃)ₘ₁ can form a dimer, trimer, tetramer, pentamer, or hexamer wherein these polymers are covalently linked among N, R₁, R₂ and R₃; and N, R₁, R₂ and/or R₃ themselves can form heterocyclic, carbocyclic, diheterocyclic, or dicarbocyclic rings.

When the preferred M is Zn, and the preferred M(NR₁R₂R₃)ₘ₁^{m2+} are exampled as following: Zn(NH₂CH₃)₂²⁺, Zn(NH₂CH₂CH₃)₂²⁺, Zn(NH₂CH₂CH₂CH₃)₂²⁺, Zn(NH₂CH(CH₃)₂)₂²⁺, Zn(NH₂C(CH₃)₃)₂²⁺, Zn(NH₂CH₂C(CH₃)₃)₂²⁺, Zn(NH(CH₃)₂)₂²⁺, Zn(NH(CH₂CH₃)₂)₂²⁺, Zn(NH(CH(CH₃)₂)₂)₂²⁺, Zn(NH(C(CH₃)₃)₂)₂²⁺, Zn(NH(CH(CH₂CH₃)₂)₂)₂²⁺, Zn(NH(CH₂C(CH₃)₃)₂)₂²⁺, Zn(NH(CH₂C(CH₂CH₃)₃)₂)₂²⁺, Zn(NH(CH₂CH₂C(CH₃)₃)₂)₂²⁺, Zn(NH₂CH₂CH₂OH)₂²⁺, Zn(NH(CH₂CH₂OH)₂)₂², Zn(N(CH₂CH₂OH)₃)₂²⁺, Zn(NH₂CH₂COOH)₂²⁺, Zn(NH₂CH₂CONH₂)₂²⁺, Zn(NH₂CH₂COOCH₃)₂²⁺, Zn(NH₂CH₂COOCH₂CH₃)₂²⁺, Zn(NH₂CH₂COOC(CH₃)₃)₂²⁺, Zn(NH₂CH₂COOCH(CH₃)₂)₂²⁺, Zn(NH₂CH₂CH₂COOH)₂²⁺, Zn(NH(CH₂COOH)₂)₂²⁺, Zn(N(CH₂CH₂COOH)₃)₂²⁺, Zn(NH₂CH₃)₄²⁺, Zn(NH₂CH₂CH₃)₄²⁺, Zn(NH₂CH₂CH₂CH₃)₄²⁺, Zn(NH₂CH(CH₃)₂)₄²⁺, Zn(NH₂C(CH₃)₃)₄²⁺, Zn(NH₂CH₂C(CH₃)₃)₄²⁺, Zn(NH(CH₃)₂)₄²⁺, Zn(NH(CH₂CH₃)₂)₄²⁺, Zn(NH(CH(CH₃)₂)₂)₄²⁺, Zn(NH(C(CH₃)₃)₂)₄²⁺, Zn(NH(CH(CH₂CH₃)₂)₂)₄²⁺, Zn(NH(CH₂C(CH₃)₃)₂)₄²⁺, Zn(NH(CH₂C(CH₂CH₃)₃)₂)₄²⁺, Zn(NH(CH₂CH₂C(CH₃)₃)₂)₄²⁺, Zn(NH₂CH₂CH₂OH)₄²⁺, Zn(NH(CH₂CH₂OH)₂)₄²⁺, Zn(N(CH₂CH₂OH)₃)₄²⁺, Zn(NH₂CH₂COOH)₄²⁺, Zn(NH₂CH₂CONH₂)₄²⁺, Zn(NH₂CH₂COOCH₃)₄²⁺, Zn(NH₂CH₂COOCH₂CH₃)₄²⁺, Zn(NH₂CH₂COOC(CH₃)₃)₄²⁺, Zn(NH₂CH₂COOCH(CH₃)₂)₄²⁺, Zn(NH₂CH₂CH₂COOH)₄²⁺, Zn(NH(CH₂COOH)₂)₄²⁺, Zn(N(CH₂CH₂COOH)₃)₄²⁺,

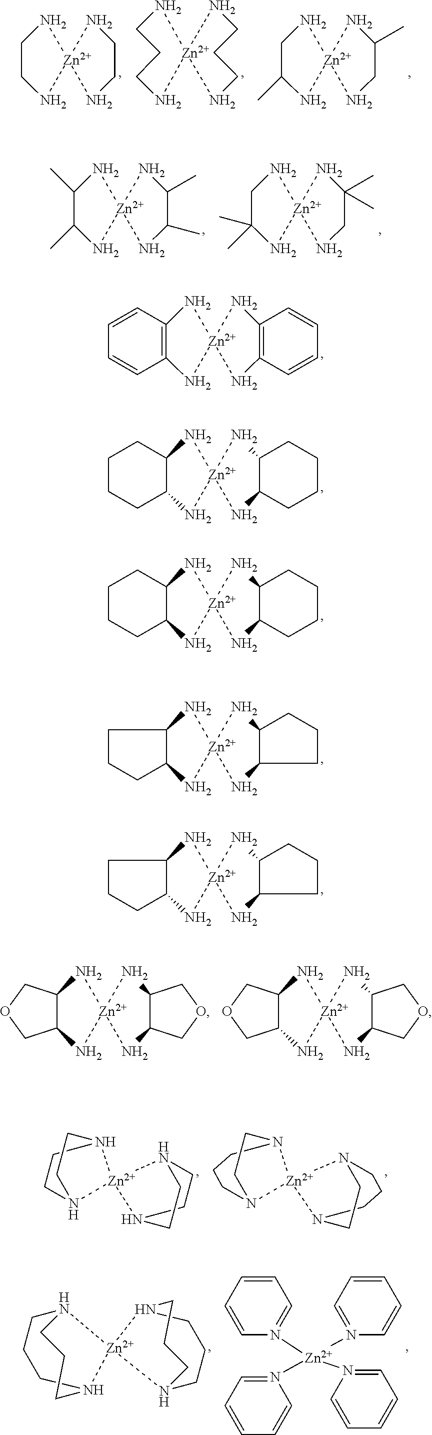

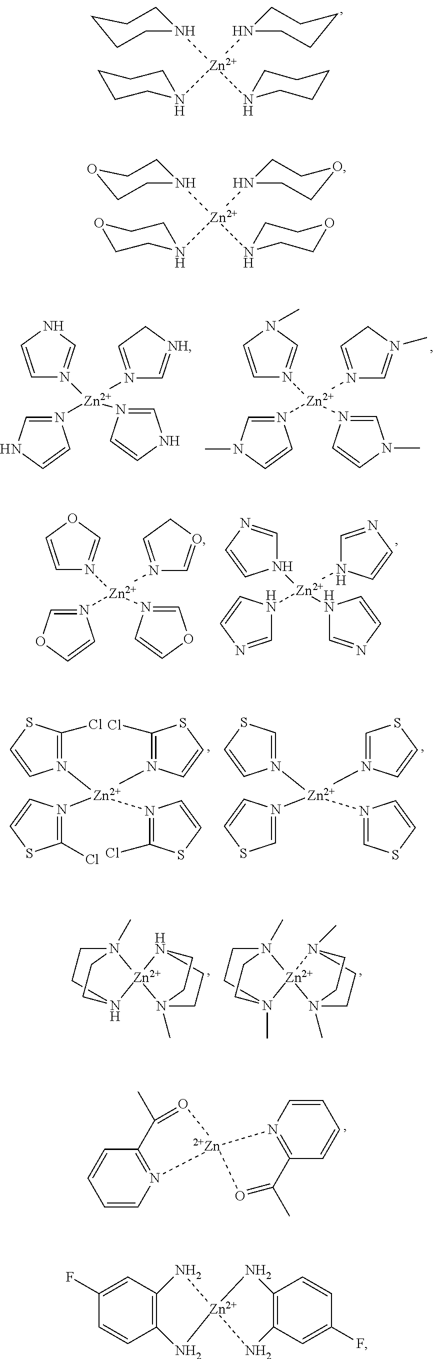

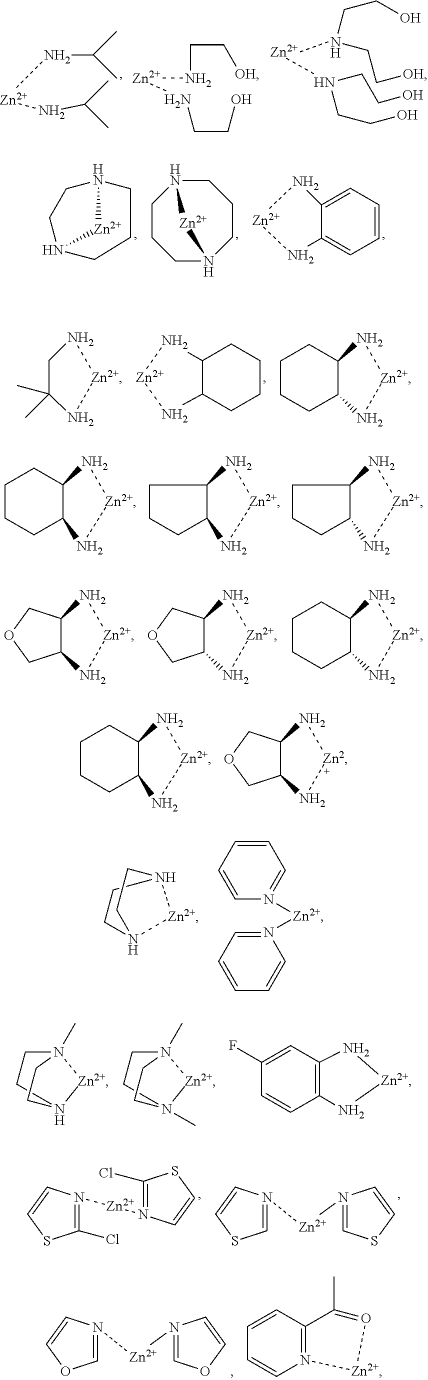

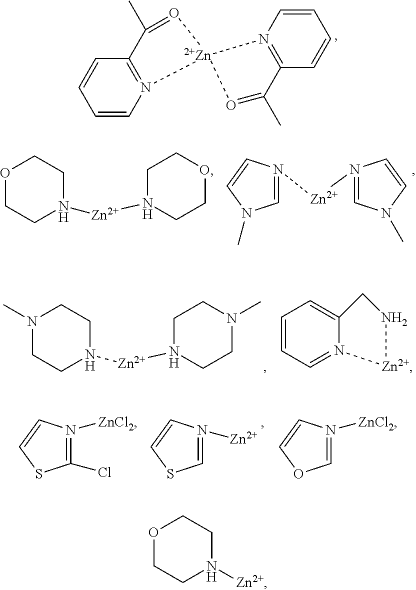

All the complex cations above can be formed with an anion, selected from, but not limited, Cl⁻, Br⁻, I⁻, SO₄²⁻, HSO₄⁻, NO₃⁻, PO₄³⁻, HPO₄²⁻, H₂PO₄⁻, CO₃²⁻, HCO₃⁻, HCOO⁻, CH₃COO⁻, F₃CCOO⁻, Cl₃CCOO⁻, FCH₂COO⁻, ClCH₂COO⁻, F₂CHCOO⁻, Cl₂CHCOO⁻, BF₄⁻, SO₃²⁻, HSO₃⁻, CH₃SO₃⁻, C₆H5CH₂SO³⁻, C₆H₅SO³⁻, C₆H₅COO⁻, C₆H₅CH₂COO⁻, C₆F₅O⁻, C₆H₄(OH)COO⁻, C₆H₂F₃O⁻, C₆H₄(NO₂)O⁻, C₆H₂(NO₂)₃O⁻, etc.

The transition metal cation-amino complex in the reaction solution are 0.5˜20 equivalents of the antibody, preferably 1.0-5.0 equivalents of the antibody, more preferably 1.5-3.0 equivalents of the antibody. The transition metal cation-amino complex can be added to the reaction solution with a water- miscible organic solvent, selected from, but not limited, ethanol, methanol, propanol, propandiol, DMA, DMF, DMSO, THF, or CH₃CN.

The reductant used in the reaction solution with the transition metal cation-amino complex is selected from Tris(2-carboxyethyl)phosphine (TCEP), (P(CH₂CH₂COOH)₃). It can be other reductants, such as Tris(hydroxypropyl)-phosphine (P(CH₂CH₂CH₂OH)₃), P(CH₂CH₃)₃, P(CH₂CH₂CH₃)₃, P(CH₂CH₂—CH₂CH₃)₃, P(CH(CH₃)₂)₃, P(CH₂CH═CH₂)₃, P(CH₂CH₂CN)₃, P(CH(CH₃)₂)₂(CH₂CH₂NH₂), P(CH₂CH₂CONH₂)₃, P(CH₂CH₂CONHCH₃)₃, P(CH₂CH₂CH₂NHCOCH₃)₃, NaB(CN)H₃, (C₆H₁₁)₂P(CH₂)₄P(C₆H₁₁)₂, (C₆H₁₁)₂P(CH₂)₃P—(C₆H₁₁)₂, Dicyclohexyl(ethyl)phosphine, Bis[2-(di-tert-butylphosphino)ethyl]amine, Tricyclohexylphosphine, 1, 2-Ethanediylbis[dicyclohexyl]-phosphine, Bis[2-(dicyclohexylphosphino)ethyl]amine, Tris[2-(diphenylphosphino)ethyl]-phosphine ([(C₆H₅)₂PCH₂CH₂]₃P), triphenylphosphine, sulfonylated triphenylphosphines (2-(diphenylphosphino)benzenesulfonic acid (diPPBS), 3-(diphenylphosphino)benzenesulfonic acid, 4-(diphenylphosphino)benzenesulfonic acid, 3, 3′, 3″-phosphinetriyltribenzenesulfonic acid). Preferably the reductant is selected from TECP or P(CH₂CH₂CH₂OH)₃, and more preferably the reductant is selected from TECP. And the concentration of the reductant in the reaction solution may be 0.04 mM-0.4 mM, or 1.0-10.0 equivalents of antibody used in the reaction. Preferably the reductant is used at 2.0-4.0 equivalents of an antibody.

The optimum buffer for conduction of the selective reduction is selected from, but not limited, PBS, Mes, Bis-Tris, Bis-Tris Propane, Pipes, Aces, Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetates, Histidine, Citrates, MES, Borates, or combinations two, three or four buffer components from above. And the pH of the buffer is selected 4.0-9.0, preferred 5.0-7.5, more preferred 5.5-7.5. The concentration of the buffer in the reaction is 0.02-1.0 M, preferably 20-200 mM, more preferably 20-100 mM. And up to 30% of water mixable (miscible) organic solvents, selected from DMA, DMF, ethanol, methanol, acetone, acetonitrile, THF, isopropanol, dioxane, propylene glycol, or ethylene diol can be added as the co-solvent in water based buffer solution;

The optimum temperature for the reduction reaction is typically controlled between about −5 and 40° C., and the reaction time is 15 minutes to 48 hours. But it is well-understandable in the field of protein conjugation that the reaction time and temperature can be determined by those skilled in the art based on the specific protein, in particular, the antibody to be conjugated. For example a preferable reduction reaction can be controlled at a temperature typically between about −5 to about 40° C., and preferably, about 0 to 37° C.; more preferably about 2 to 8° C., and more procisily 4±1° C. The process of the conjugation is 15 min to 12 hours, and more preferably at a temperature between about 2 and 8° C., and the process time is about 30 min to 15 hours (overnight).

During the reduction, or after the reduction, a Drug/linker complex/assembly is directly added to the solution of the reduction reaction for conjugation. The Drug/linker complex/assembly, having a formula (I) or (II) represented as:

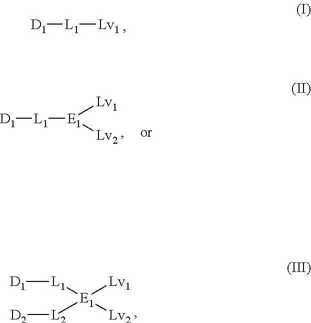

wherein: Lv₁ and Lv₂ are a thiol reaction group, and are independently selected from:

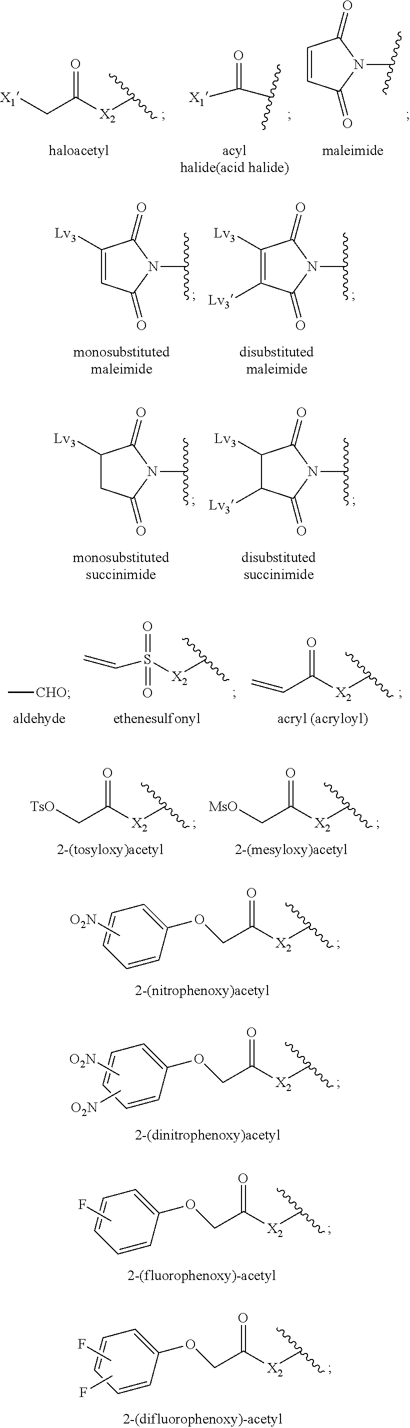

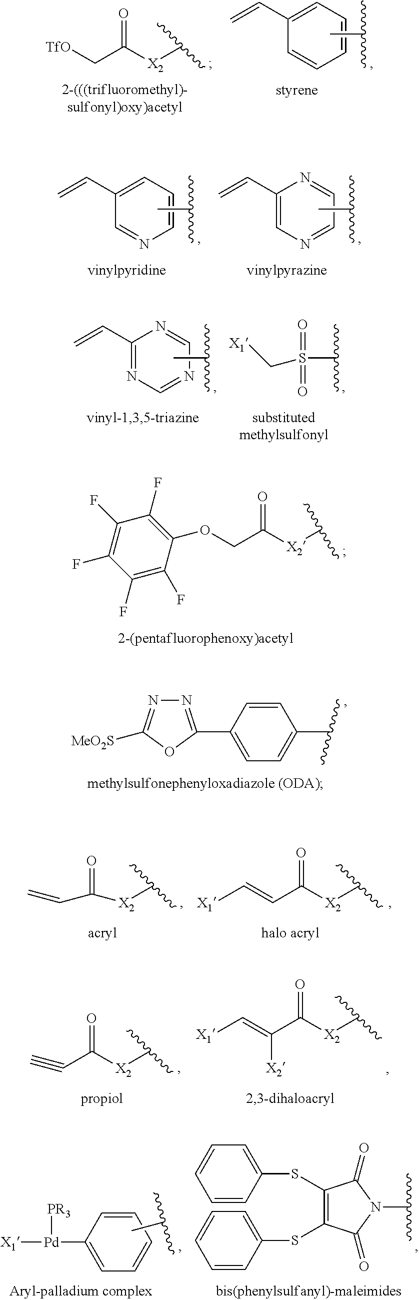

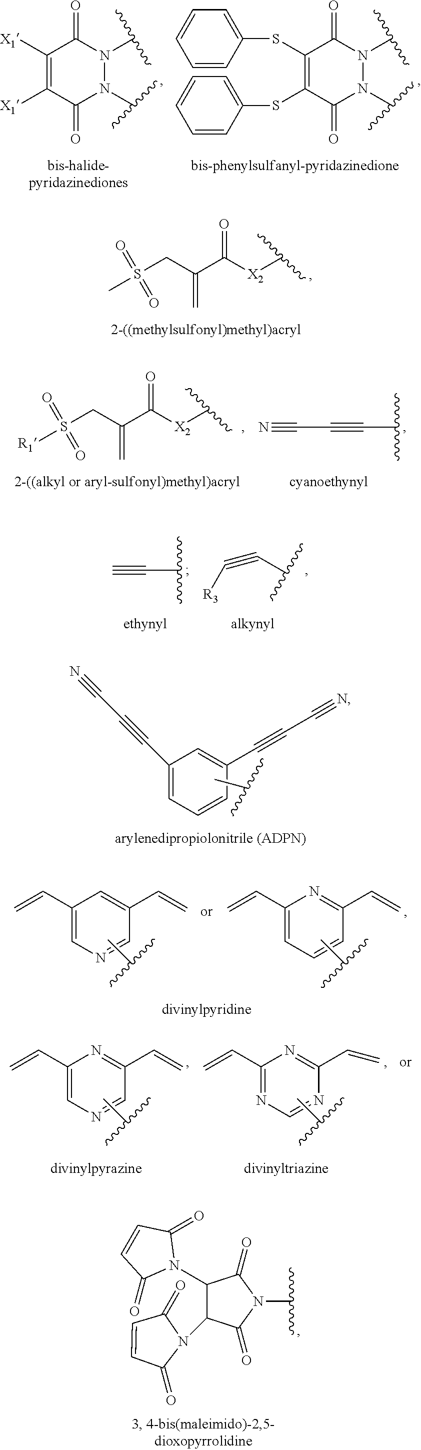

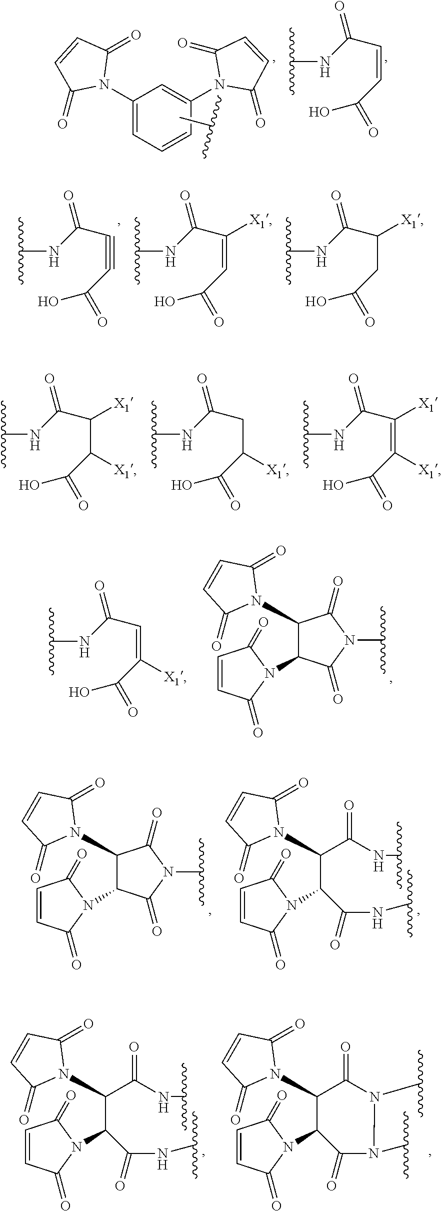

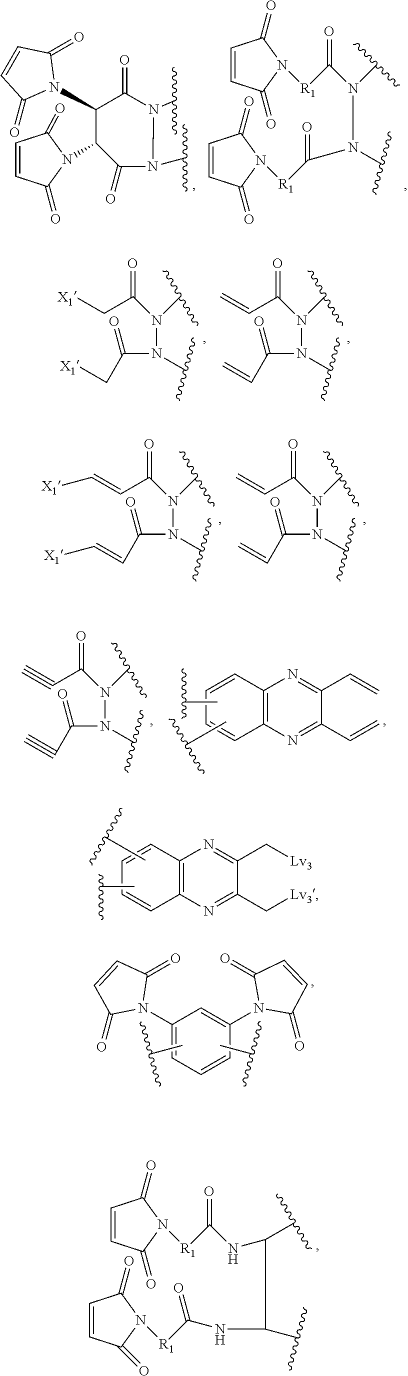

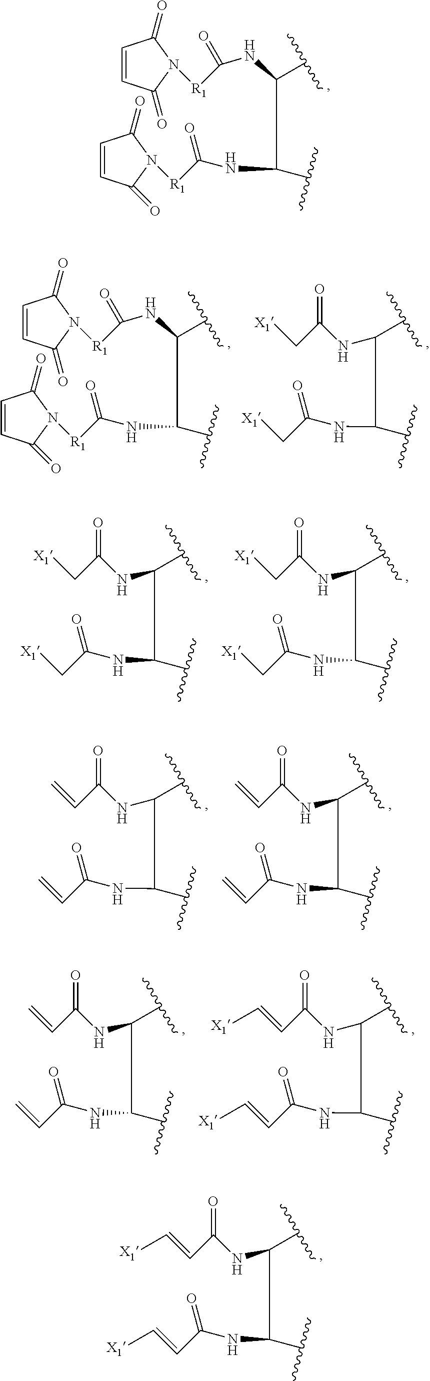

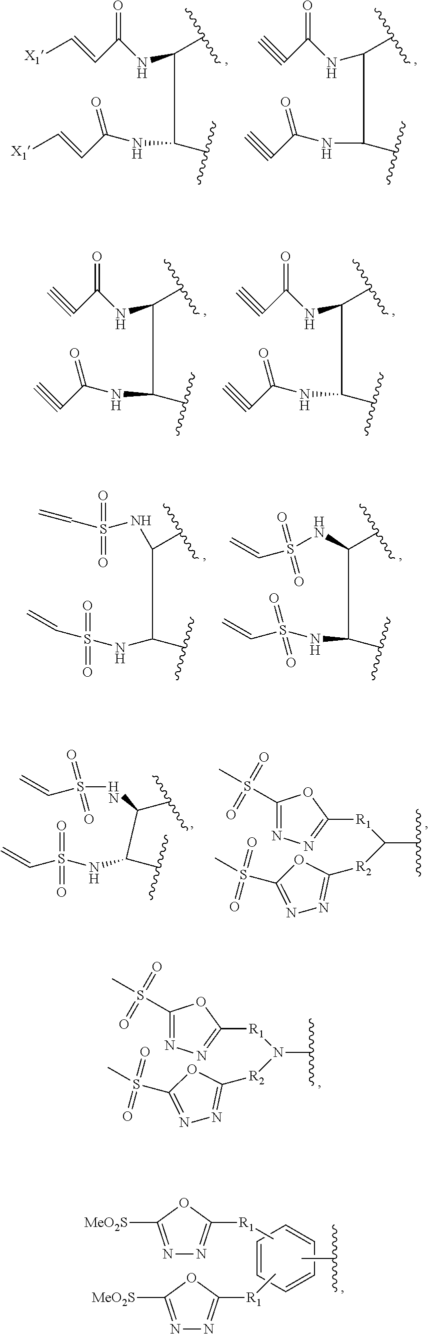

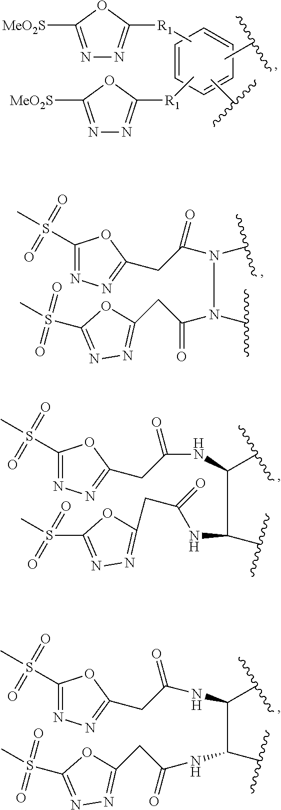

wherein X₁′ and X^{2′} are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

In the formula (II) and formula (III) wherein

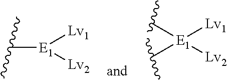

can be selected from:

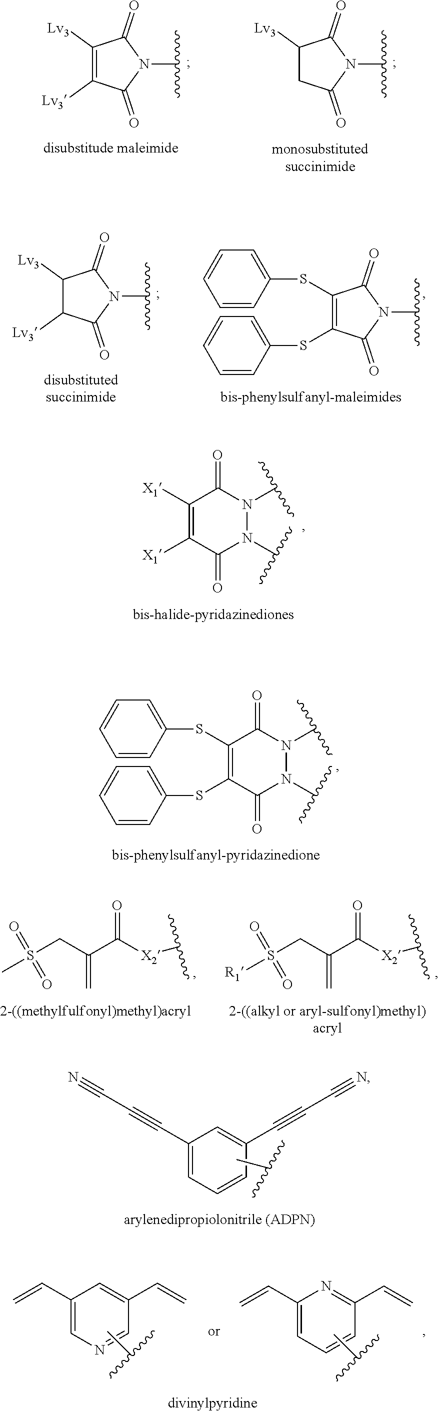

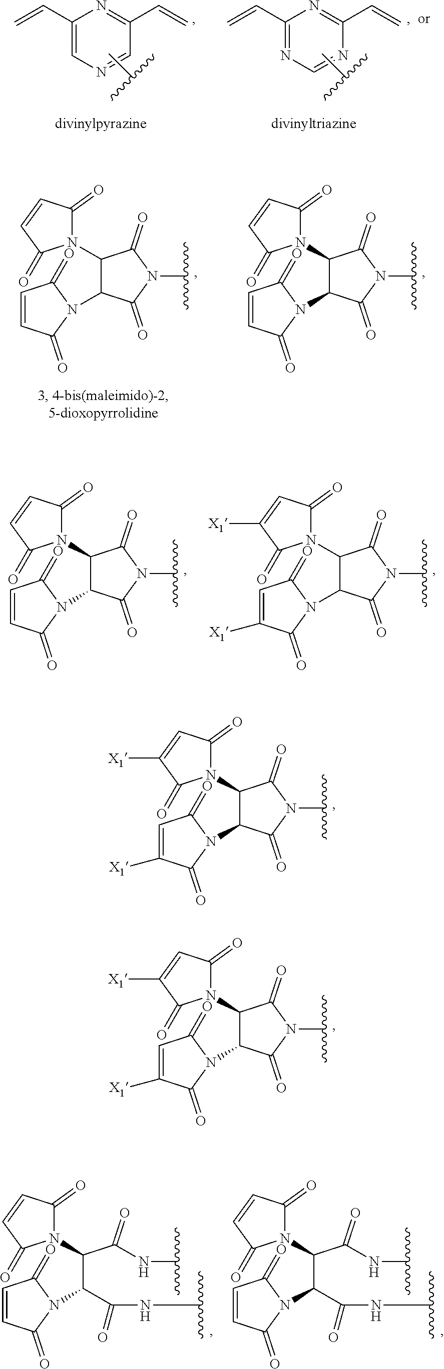

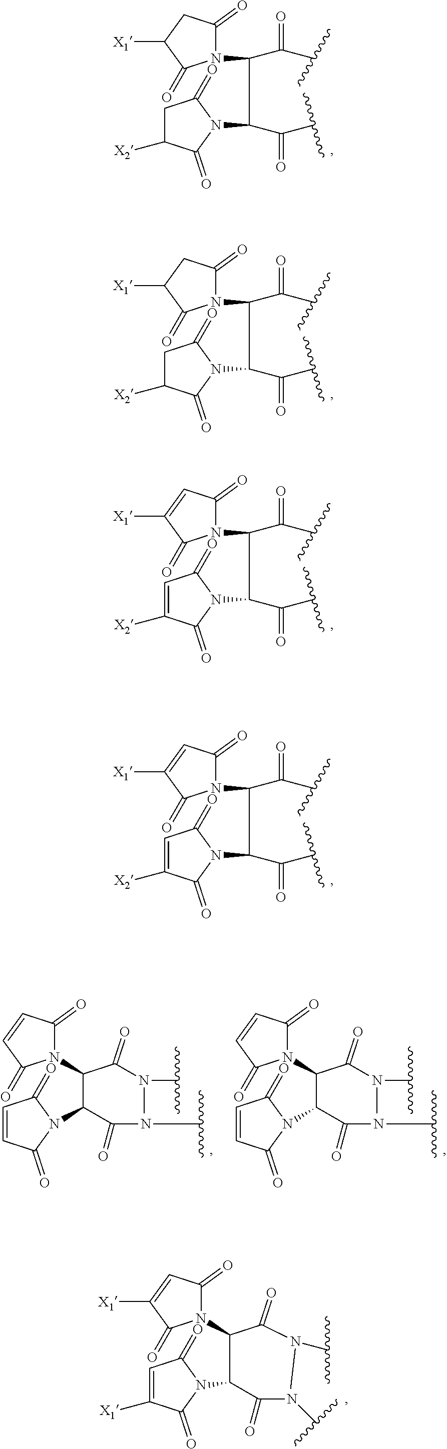

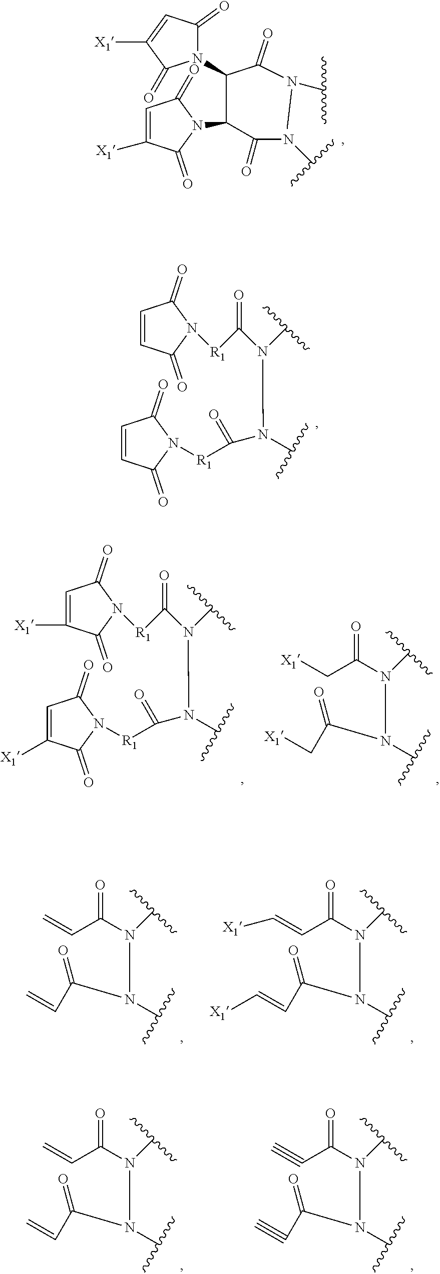

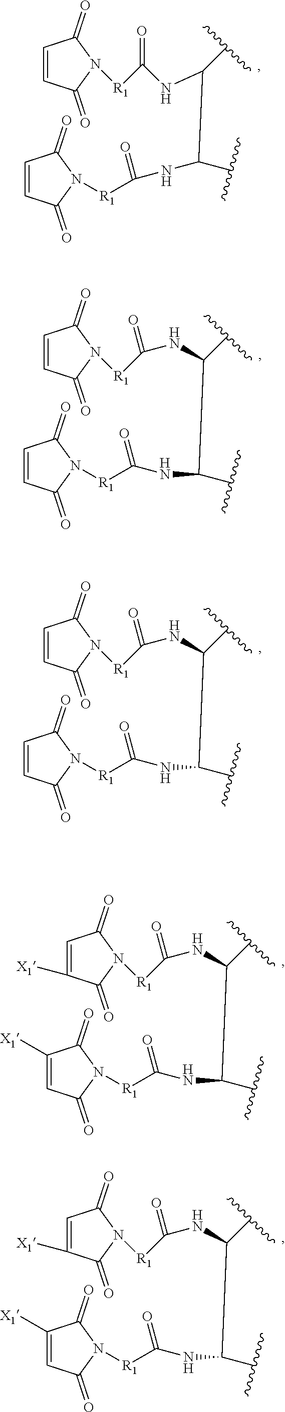

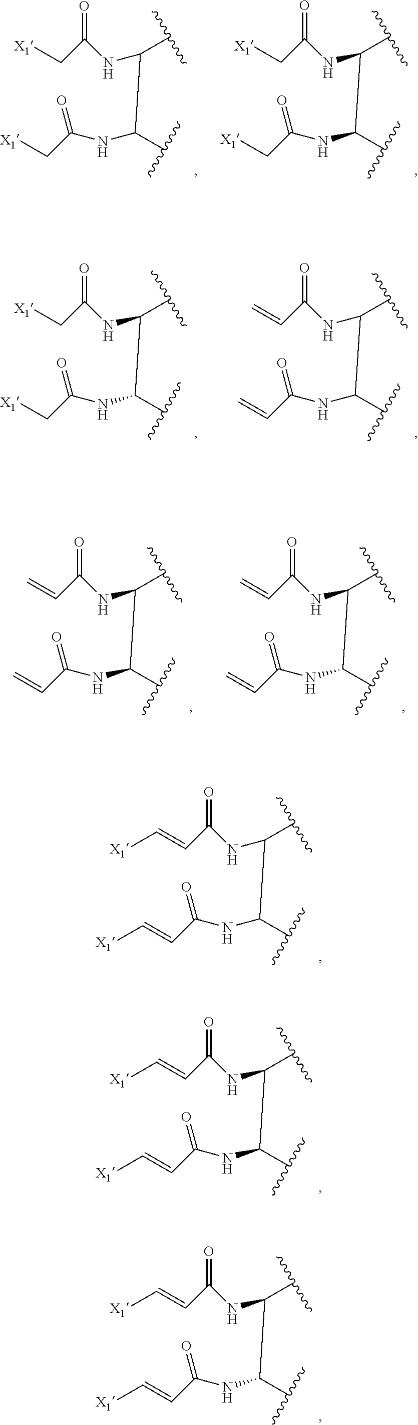

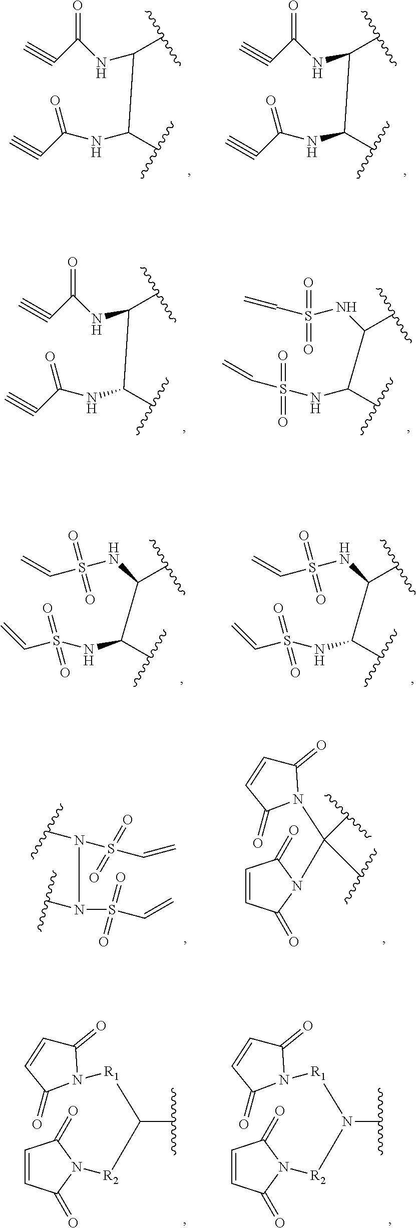

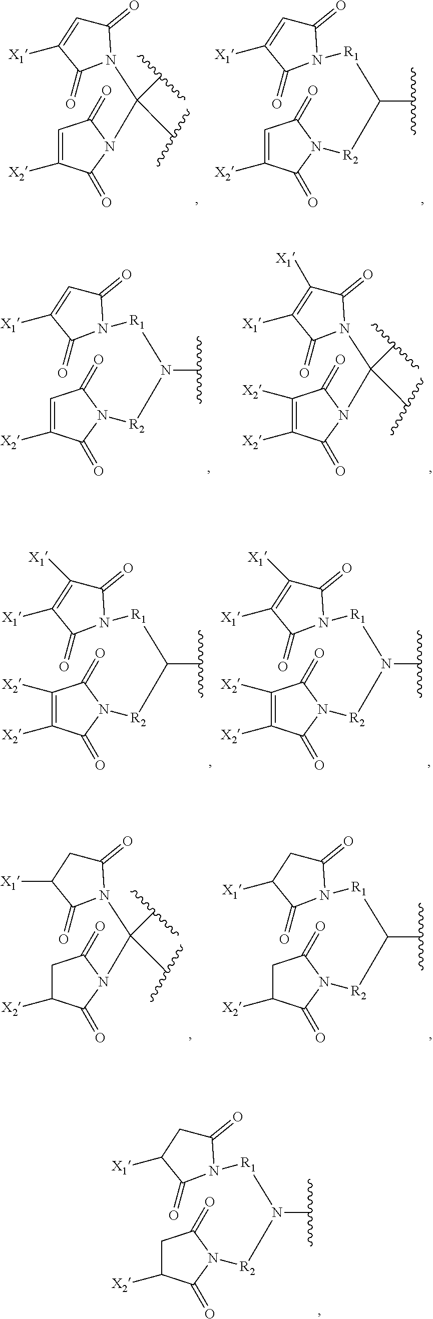

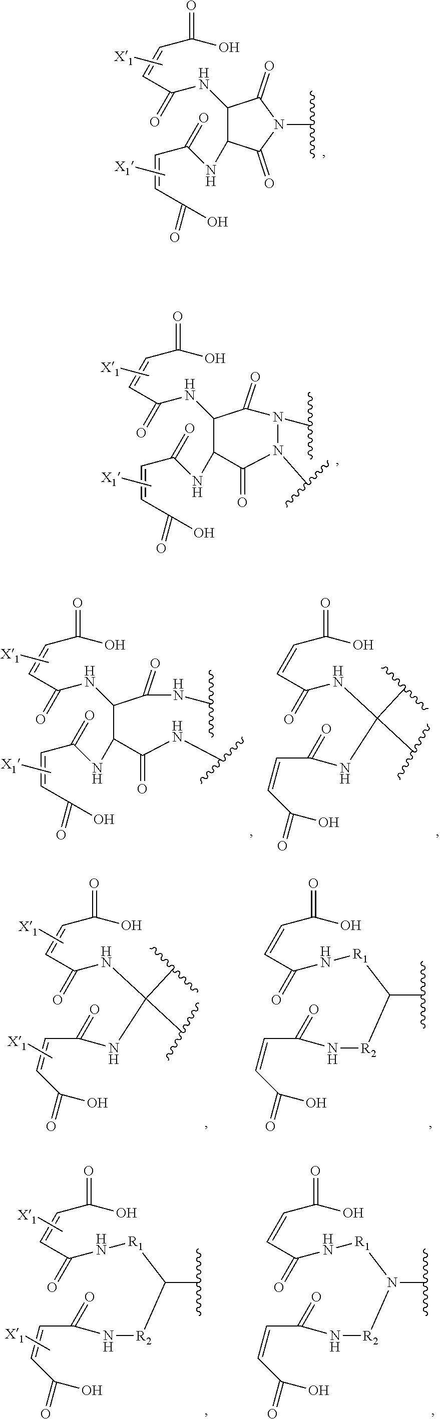

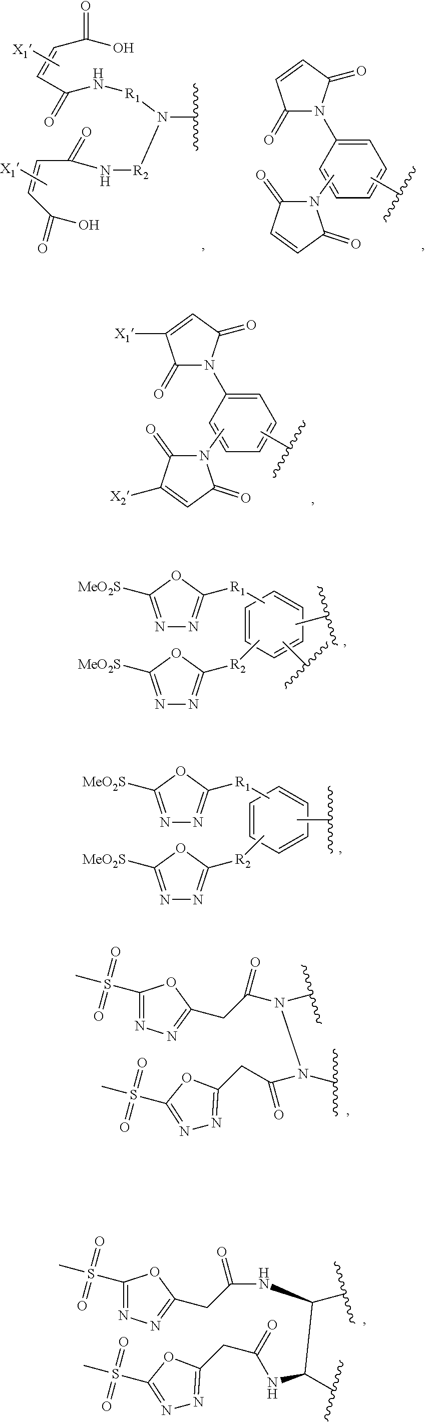

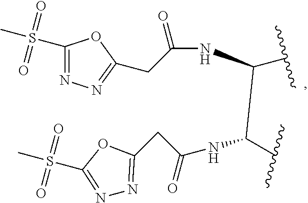

wherein Lv₃, Lv₃′, X₁′ and X^{2′} are described above; the connecting bond “ [symbol] ” in the middle of the two atoms means it can link either of the two atoms.

L₁ and L₂ are, the same or different, independently selected from O, NH, S, NHNH, N(R₃), N(R₃)N(R^{3′}), polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)_{p′}R^{3′}], or (OCH₂CH₂)ₚCOOR₃, or CH₂CH₂(OCH₂CH₂)ₚCOOR₃, wherein p and p′ are independently an integer selected from 0 to about 1000, or combination thereof, C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; Wherein R₃ and R^{3′} are independently H; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or 1-8 natural or unnatural amino acids described in the definition; or polyethyleneoxy unit of formula (OCH₂CH₂)ₚ or (OCH₂CH(CH₃))ₚ, wherein p is an integer from 0 to about 1000, or combination above thereof.

L₁ or L₂ may contain a self-immolative or a non-self-immolative component, peptidyl units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals.

Preferably, the self-immolative linker component has one of the following structures:

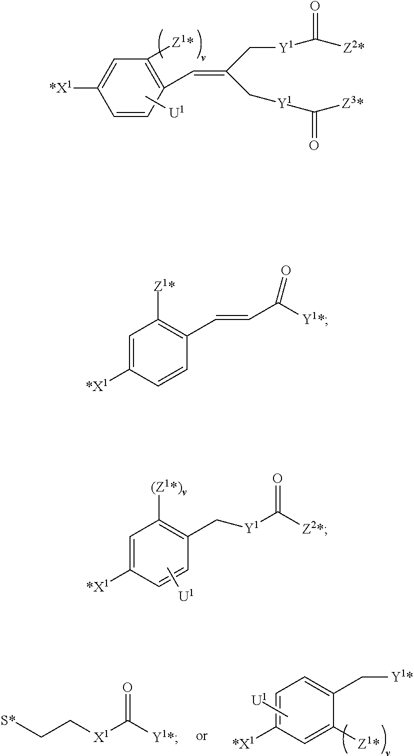

wherein the (*) atom is the point of attachment of additional spacer or releasable linker units or the cytotoxic agent, and/or the binding molecule (CBA); X¹, Y¹, Z² and Z³ are independently NH, 0, or S; Z¹ is independently H, NH, 0 or S; v is 0 or 1; U¹ is independently H, OH, C₁-C₆ alkyl, (OCH₂CH₂)ₙF, C₁, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′) wherein R₅ and R₅′ are as defined above; preferably R₅ and R₅′ are independently selected from H, C₁-C₈ alkyl; C₂˜C₈ alkenyl, alkynyl, heteroalkyl; C₃-C₈ aryl, heterocyclic, carbocyclic, cycloalkyl, heterocycloalkyl, heteroaralkyl, alkylcarbonyl; or pharmaceutical cation salts.

The non-self-immolative linker component is one of the following structures:

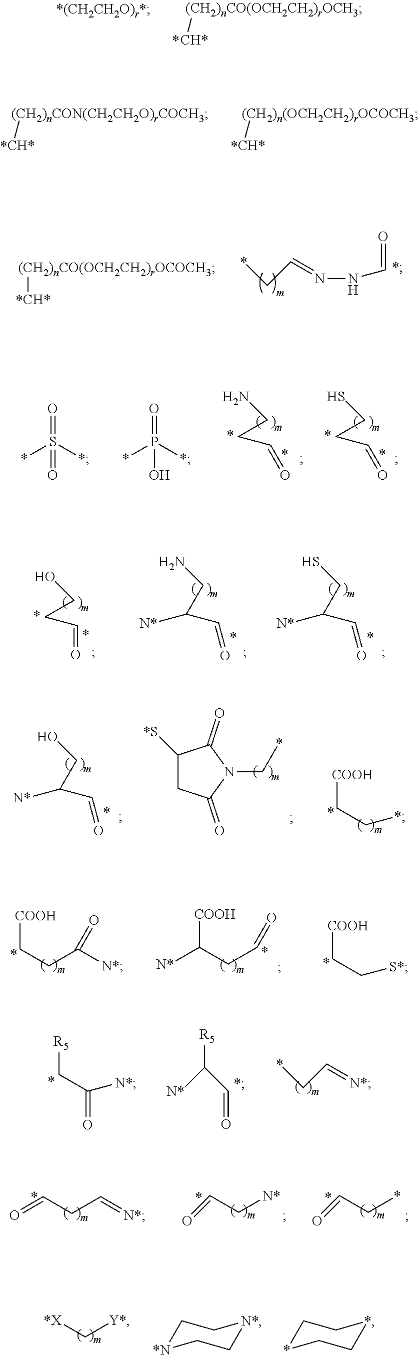

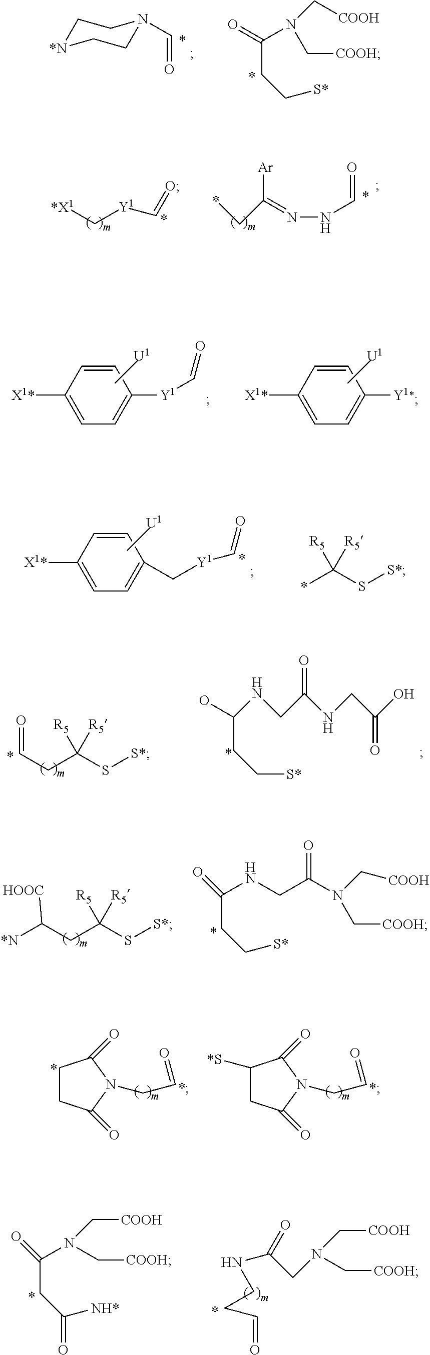

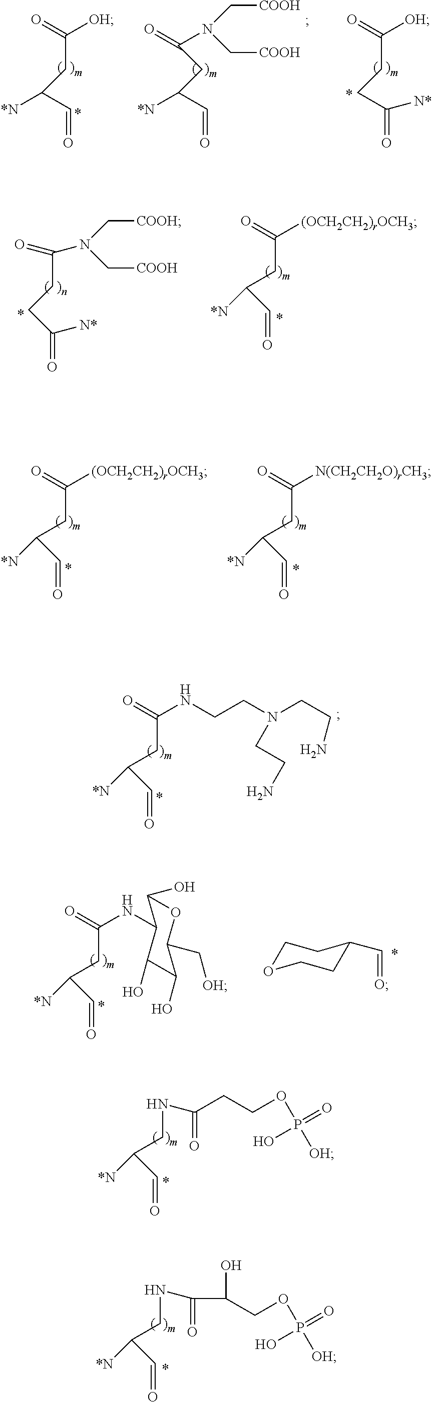

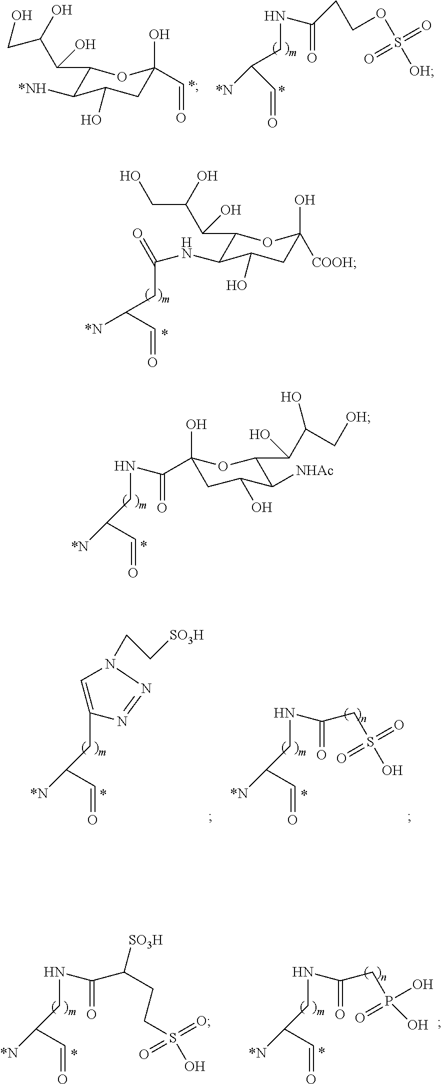

Wherein the (*) atom is the point of attachment of additional spacer R₁ or releasable linkers, the cytotoxic agents, and/or the binding molecules; X¹, Y¹, U¹, R₁, R₅, R₅′ are defined as above; r is 0˜100; m and n are 0˜6 independently.

More preferably, L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites.

Further preferably, L₁ or L₂ may be a releasable linker. The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells.

Examples of the releasable linkers (L, Lᵢ or L₂) include, but not limited:
—(CR₅R₆)ₘ(Aa)r(CR₇R₈)ₙ(OCH₂CH₂)t-, —(CR₅R₆)ₘ(CR₇R₈)ₙ(Aa)ᵣ(OCH₂CH₂)—, -(Aa)ᵣ-(CR₅R₆)m(CR₇R₈)n(OCH₂CH₂)ₜ—, —(CR₅R₆)ₘ(CR₇R₈)ₙ(OCH₂CH₂)r(Aa)ₜ-, —(CR₅R₆)ₘ—(CR₇=CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)t(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ.(CR₉R₁₀)ₙ(OCH₂CH₂)ᵣ—, —(CR₅R₆)ₘ(NR₁₁CO)(Aa)t(CR₉R₁₀)ₙ(OCH₂CH₂)ᵣ—, —(CR₅R₆)ₘ—(OCO)(Aa)t(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)r, —(CR₅R₆)ₘ(CO)(Aa)ₜ(CR₉R₁₀)ₙ(OCH₂CH₂)r, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)(CR₇R₈)ₙ—, —(CR₅R₆)ₘthiazolyl-CO-(Aa)ₜ(CCR₇R₈)ₙ—, —(CR₅R₆)ₜ-thienyl-CO(CR₇R₈)ₙ—, —(CR₅R₆)t-imidazolyl-CO—(CR₇R₈)ₙ—, —(CR₅R₆)-morpholino-CO(Aa)ₜ-(CR₇R₈)ₙ—, —(CR₅R₆)tpiperazino-CO(Aa)ₜ(CR₇R₈)ₙ—, —(CR₅R₆)t-N-methylpiperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)-, —(CR₅R₆)ₘ-thienyl-(Aa)ₜ-, —(CR₅R₆)ₘ-imidazolyl(Aa)ₜ-, —(C R₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)r(CR₇R₈)n(OCH₂CH₂)ₜ—K(CR₅R₆)ₘ(CR₇R₈)ₙ-(Aa)ᵣ(OCH₂CH₂)ₜ—, —K(Aa)ᵣ(CR₅R₆)ₘ(CR₇R₈)ₙ(OCH₂CH₂)—K(CR₅R₆)ₘ(CR₇R₈)ₙ—(OCH₂CH₂)r(Aa)ₜ-, —K(CR₅R₆)ₘ(CR₇=CR₈)(CR₉R₁₀)ₙ(Aa)ₜ(OCH₂CH₂)r-, —K(CR₅R₆)ₘ—(NR₁CO)(Aa)ₜ(CR₉R₁₀)n(OCH₂CH₂)ᵣ—K(CR₅R₆)ₘ(Aa)ₜ(NR₁₁CO)(CR₉R₁₀)n(OCH₂CH₂)r-, —K(CR₅R₆)ₘ(OCO)(Aa)ₜ(CR₉R₁₀)ₙ. (OCH₂CH₂)ᵣ—, —K(CR₅R₆)ₘ(OCNR₇)(Aa)ₜ(CR₉R₁₀)n-(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)-(Aa)ₜ(CR₉R₁₀)˜(OCH₂CH₂)ᵣ—, —K(CR₅R₆)ₘ—(OCO)(Aa)ₜ(CR₉R₁₀)n(OCH₂CH₂)ᵣ—, —K(CR₅R₆)ₘ(OCNR₇)(Aa)ₜ(CR₉R₁₀)n(OCH₂CH₂)ᵣ—, —K(CR₅R₆)ₘ(CO)(Aa)ₜ(CR₉R₁₀)ₙ—(OCH₂CH₂)ᵣ—, —K(CR₅R₆)ₘ-phenyl-CO(Aa)ₜ(CR₇R₈)n-, —K—(CR₅R₆)ₘ-furyl-CO(Aa)ₜ(CR₇R₈)₁—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)timidazolyl-CO—(CR₇R₈)ₙ—, —K(CR₅R₆)morpholino-CO(Aa)ₜ(CR₇R₈)₁—, —K(CR₅R₆)piperazino-CO(Aa)ₜ(CR₇R₈)˜-, —K(CR₅R₆)ₜ—N-methylpiperazinCO(Aa)ₜ(CR₇R₈)ᵣ—, —K(CR₅R)ₘ(Aa)ₜphenyl, —K—(CR₅R₆)ₘ-(Aa)ₜfuryl-, —K(CR₅R₆)ₘ-oxazolyl(Aa)ₜ-, —K(CR₅R₆)ₘ-thiazolyl(Aa)-, —K(CR₅R₆)ₘ-thienyl-(Aa)-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, n, R₃, R₄, and R₅ are described above; t and r are 0-100 independently; R₆, R₇, and Rₛ are independently chosen from H; halide; C₁-C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group.

Example structures of the components of the linker L₁ and L₂ may contain:

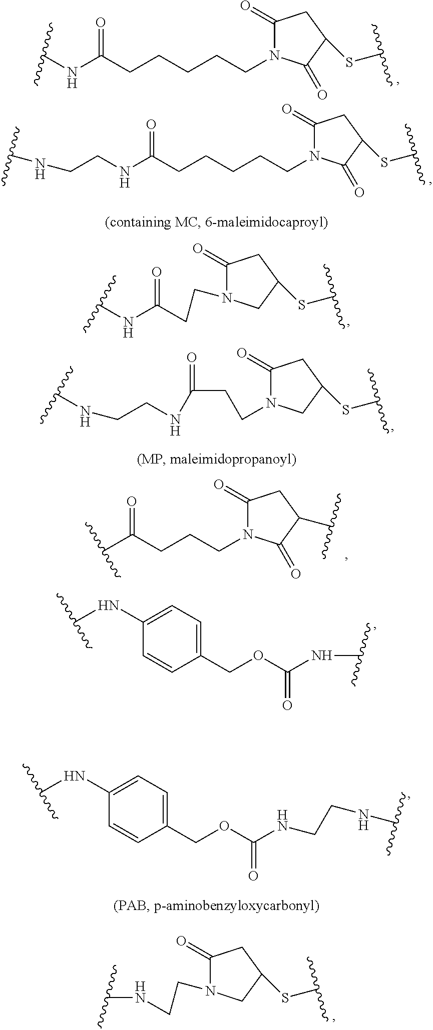

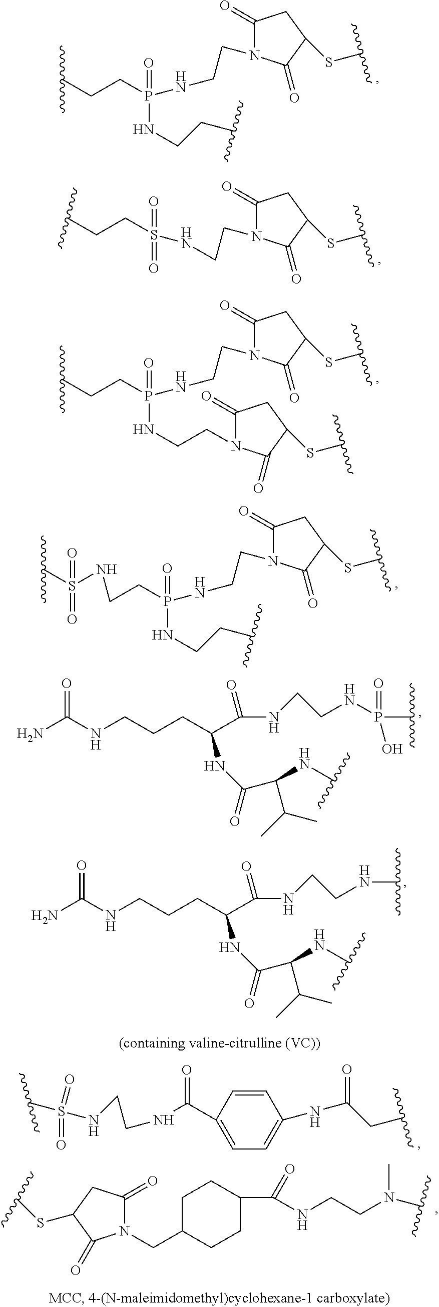

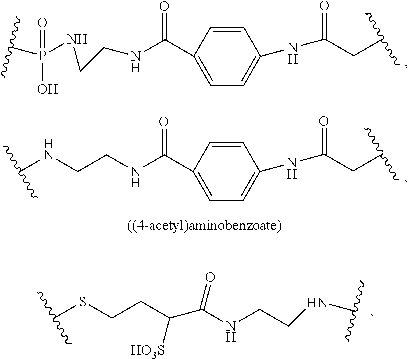 [SMILES: *CC(=O)Nc1ccc(C(=O)NCCNP(*)(=O)O)cc1 ; *CC(=O)Nc1ccc(C(=O)NCCN*)cc1 ; *NCCNC(=O)C(CCS*)S(=O)(=O)O]

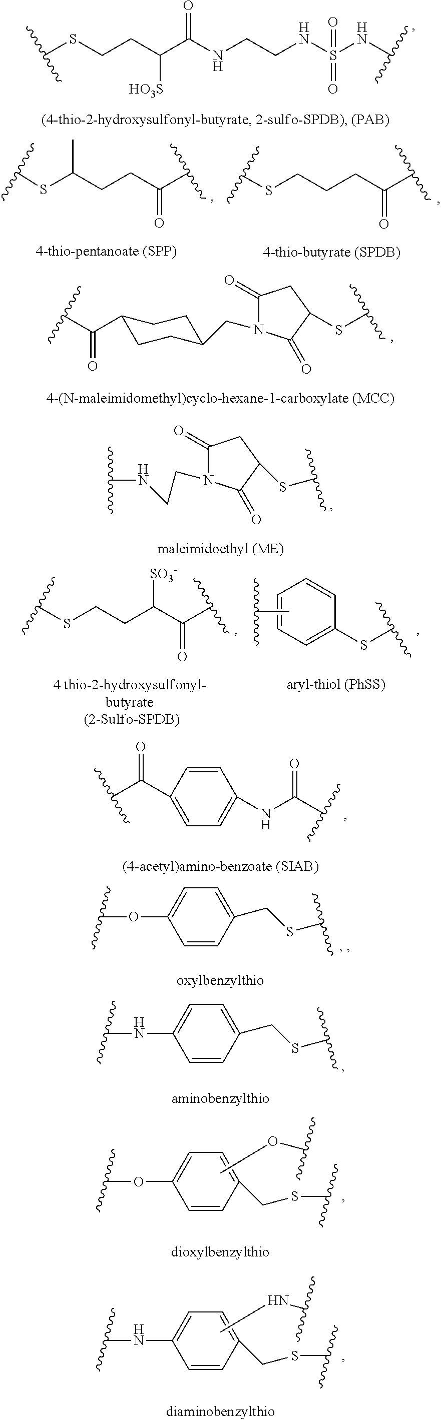

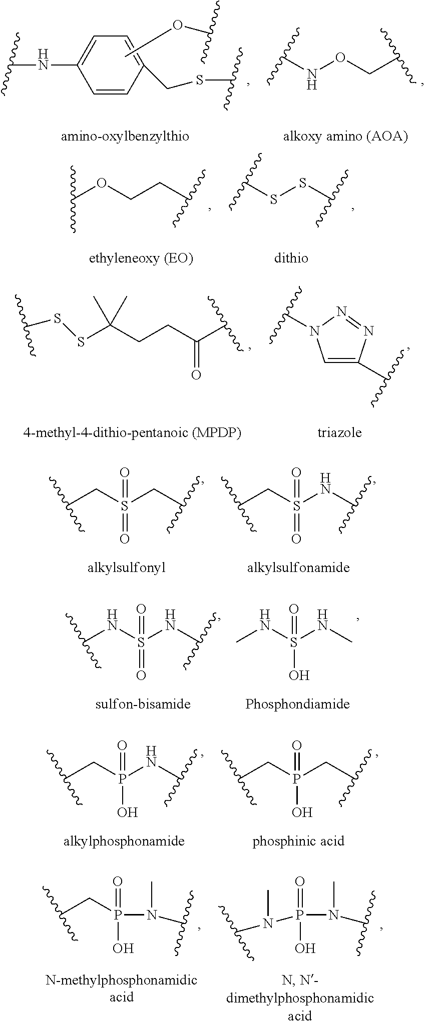

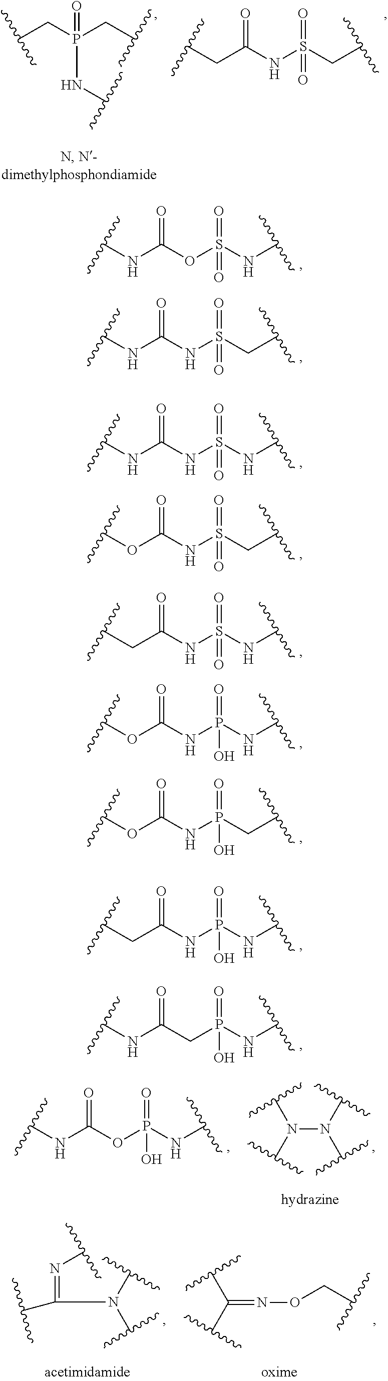

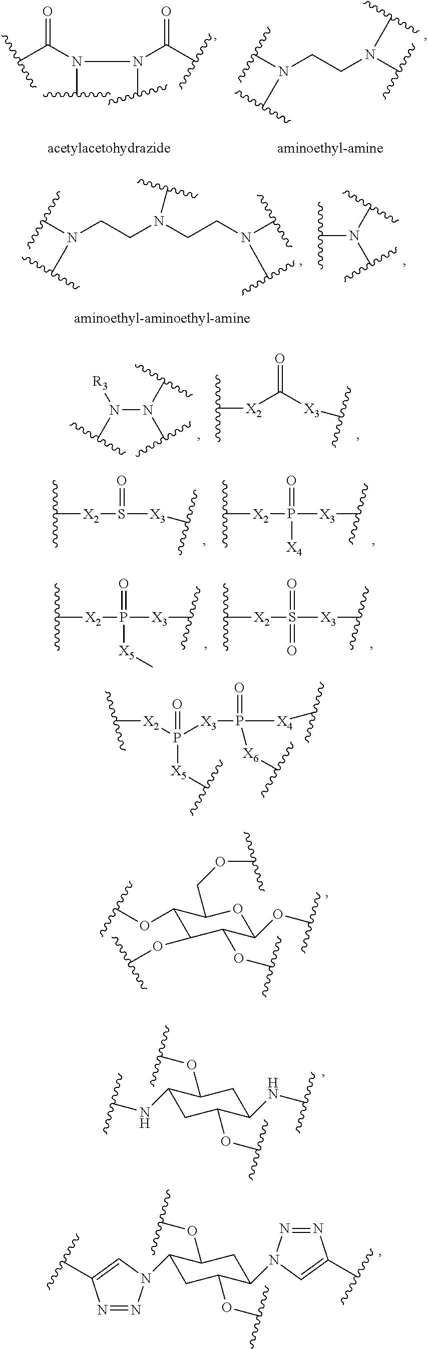

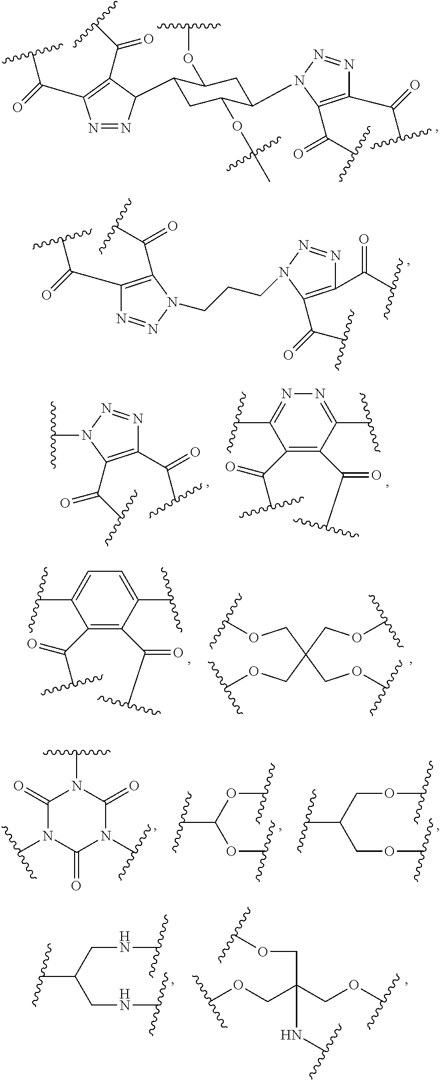

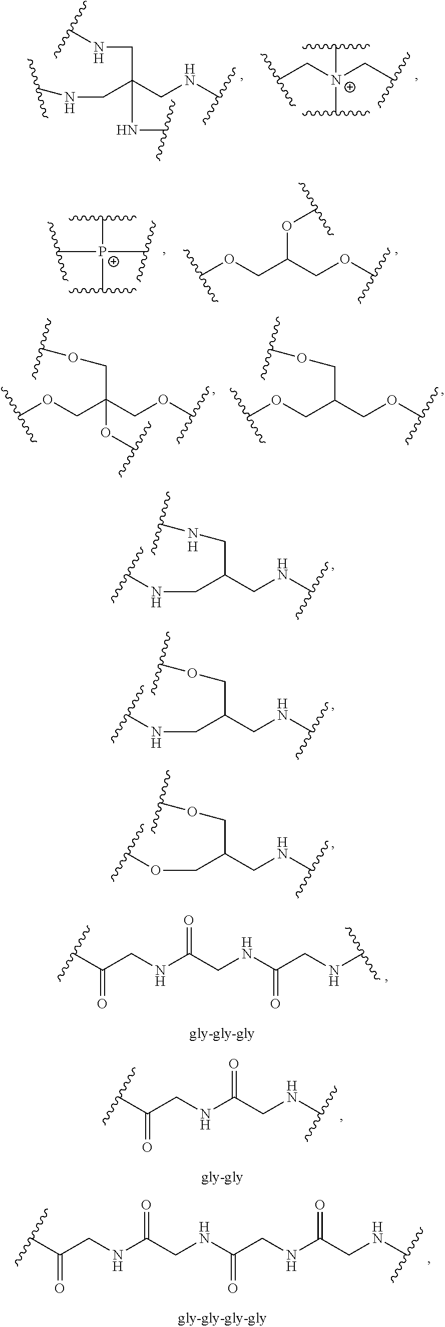

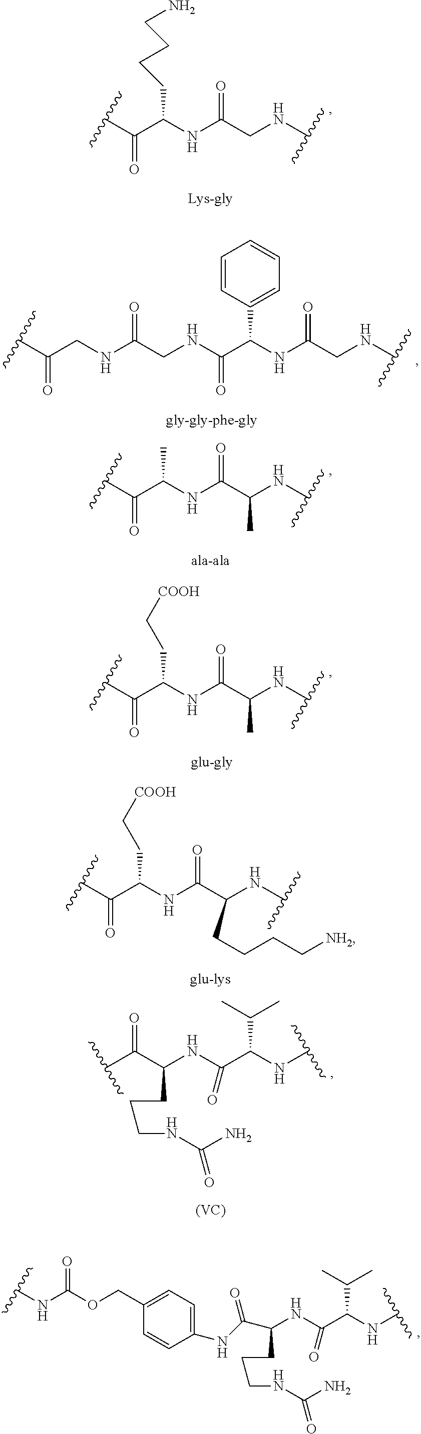

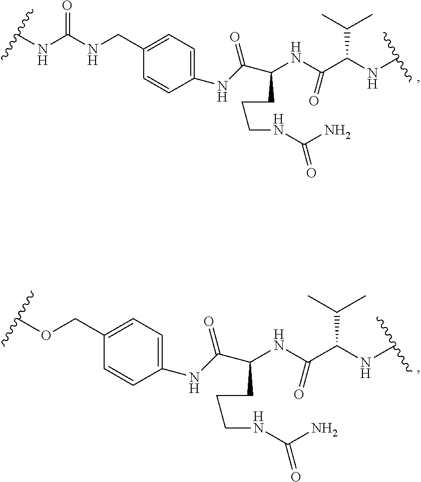 [SMILES: *NC(=O)NCc1ccc(NC(=O)[C@H](CCCNC(N)=O)NC(=O)[C@@H](N*)C(C)C)cc1 ; *N[C@H](C(=O)N[C@@H](CCCNC(N)=O)C(=O)Nc1ccc(CO*)cc1)C(C)C]

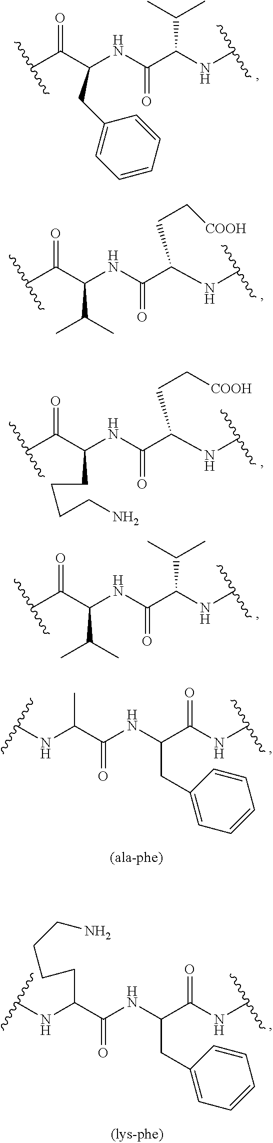

or a combination above thereof; wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

In addition, L₁, L₂, X₁, X₂, X₃, X₁′, X^{2′} and X₃′ can be independently absent.

E₁ is a joint group that link two thiol reactonable groups of Lv₁ and Lv₂. E₁ is selected from CH, CH₂, NH, NHNH, N(R₃), N(R₃)N(R^{3′}), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1˜4 units of aminoacids, preferably selected from aspatic acid, glutamic acid, arginine, histidine, lysine, serine, threonine, asparagine, glutamine, cysteine, selenocysteine, tyrosine, phenylalanine, glycine, proline, tryptophan, alanine; or one of the following structures:

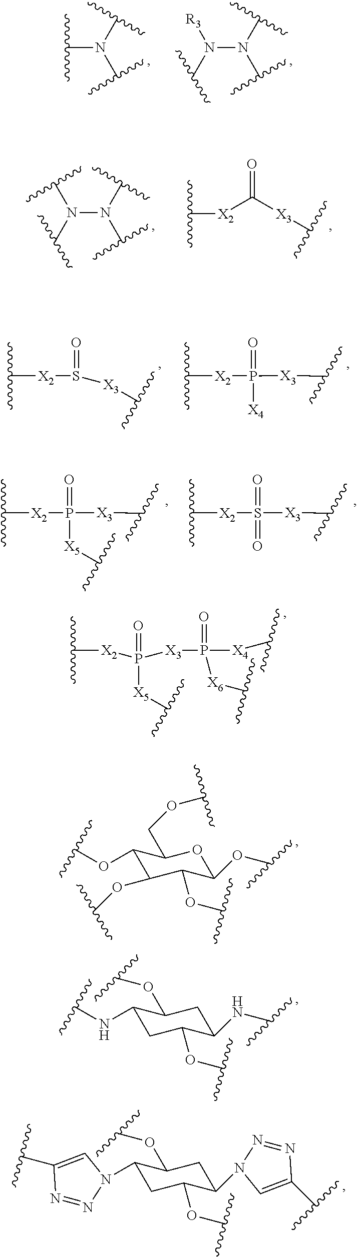

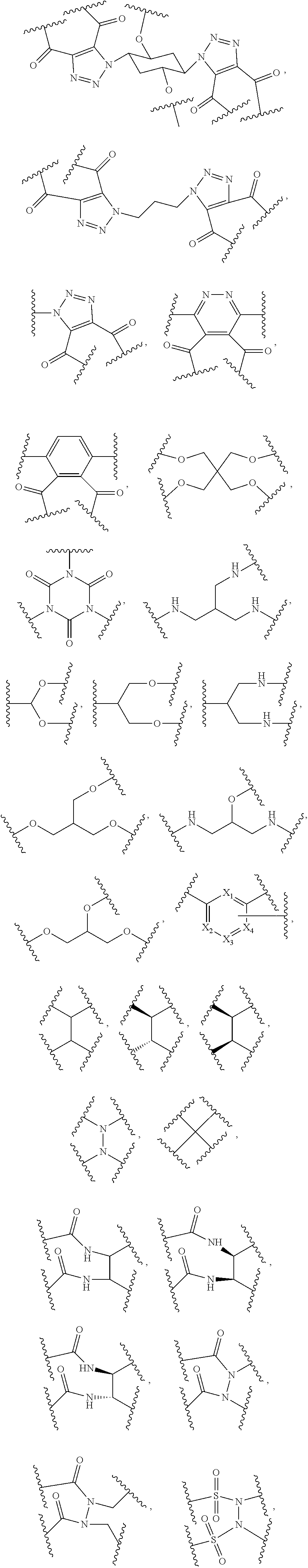

wherein [symbol] is the site of linkage;
D is a cytotoxic drug, or a therapeutic drug, or an immunotherapeutical protein, or a function molecule for enhancement of binding or stabilization of the cell-binding protein agent, or a cell-surface receptor binding lingand, such as an antybody or an antibody fragment, or siRNA or DNA molecule.

The cytotoxic drug is selected from, but not limited to:
1). Chemotherapeutic agents: a). Alkylating agents: such as Nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189 and CBI-TMI); Benzodiazepine dimers (e.g., dimmers of pyrrolobenzodiazepine (PBD) or tomaymycin, indolinobenzodiazepines, imidazobenzothiadiazepines, or oxazolidino-benzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemel-amine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermo-lide, bryostatins, dolostatins, auristatins, tubulysins, cephalostatins; pancratistatin; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C)]; d). Anti-metabolites: such as {[Anti-folate: DIFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; e). Hormonal therapies: such as {Receptor antagonists: [Anti-estrogen: (megestrol, raloxifene, tamoxifen); LURH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; f). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, pirarubicin, zorubicin, mtoxantrone, MDR inhibitors (such as verapamil), Ca²⁺ ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine, razoxane; rhizoxin; sizofiran; spirogermanium; tenuazonic acid; triaziquone; 2, 2′, 2″-trichlorotriethylamine; trichothecenes (especially T-2 toxin, verrucarin A, roridin A and anguidine); urethane, siRNA, antisense drugs, and a nucleolytic enzyme.
2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.
3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols:azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). (3-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; 1). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;
4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′, 3′-dideoxythymidine (FLT) and 3′-fluoro-2′, 3′-dideoxyguanosine (FLG), fomivirsen, ganciclovir, idoxuridine, lamivudine (3TC), 1-nucleosides (e.g. β-1-thymidine and p-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.
5). The drugs used for conjugates via a bridge linker of the present invention also include radioisotopes. Examples of radioisotopes (radionuclides) are 3H, C, 1C, F, 32P 35, 64Cu, ⁸Ga, 86Y 99Tc, In, 123I 124I 125I 131 133Xe, ¹⁷⁷Lu, ²¹¹At, or ²¹³Bi. Radioisotope labeled antibodies are useful in receptor targeted imaging experiments or can be for targeted treatment such as with the antibody-drug conjugates of the invention (Wu et al (2005) Nature Biotechnology 23(9): 1137-46). The cell binding molecules, e.g. an antibody can be labeled with ligand reagents through the bridge linkers of the present patent that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).
6). The pharmaceutically acceptable salts, acids or derivatives of any of the above drugs.

In another embodiment, the drug D can be a chromophore molecule, for which the conjugate can be used for detection, monitoring, or study the interaction of the cell binding molecule with a target cell. Chromophore molecules are a compound that have the ability to absorb a kind of light, such as UV light, florescent light, IR light, near IR light, visual light; A chromatophore molecule includes a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

The chromophore molecule can be selected from, but not limited, non-protein organic fluorophores, such as: Xanthene derivatives (fluorescein, rhodamine, Oregon green, eosin, and Texas red); Cyanine derivatives: (cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (dansyl and prodan derivatives); Coumarin derivatives; Oxadiazole derivatives (pyridyloxazole, nitrobenzoxadiazole and benzoxadiazole); Anthracene derivatives (anthraquinones, including DRAQ5, DRAQ7 and CyTRAK Orange); Pyrene derivatives (cascade blue, etc); Oxazine derivatives (Nile red, Nile blue, cresyl violet, oxazine 170 etc). Acridine derivatives (proflavin, acridine orange, acridine yellow etc). Arylmethine derivatives (auramine, crystal violet, malachite green). Tetrapyrrole derivatives (porphin, phthalocyanine, bilirubin).

Or a chromophore molecule can be selected from any analogs and derivatives of the following fluorophore compounds: CF dye (Biotium), DRAQ and CyTRAK probes (BioStatus), BODIPY (Invitrogen), Alexa Fluor (Invitrogen), DyLight Fluor (Thermo Scientific, Pierce), Atto and Tracy (Sigma Aldrich), FluoProbes (Interchim), Abberior Dyes (Abberior), DY and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (APC, RPEPerCP, Phycobilisomes)(Columbia Biosciences), APC, APCXL, RPE, BPE (Phyco-Biotech).

Examples of the widely used fluorophore compounds which are reactive or conjugatable with the linkers of the invention are: Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, IR-783, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine.

The fluorophore compounds that can be linked to the linkers of the invention for study of nucleic acids or proteins are selected from the following compounds or their derivatives: 7-AAD (7-aminoactinomycin D, CG-selective), Acridine Orange, Chromomycin A3, CyTRAK Orange (Biostatus, red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1. The fluorophore compounds that can be linked to the linkers of the invention for study cells are selected from the following compounds or their derivatives: DCFH (2′7′Dichorodihydro-fluorescein, oxidized form), DHR (Dihydrorhodamine 123, oxidized form, light catalyzes oxidation), Fluo-3 (AM ester. pH >6), Fluo-4 (AM ester. pH 7.2), Indo-1 (AM ester, low/high calcium (Ca2+)), and SNARF (pH 6/9). The preferred fluorophore compounds that can be linked to the linkers of the invention for study proteins/antibodies are selected from the following compounds or their derivatives: Allophycocyanin (APC), AmCyan1 (tetramer, Clontech), AsRed2 (tetramer, Clontech), Azami Green (monomer, MBL), Azurite, B-phycoerythrin (BPE), Cerulean, CyPet, DsRed monomer (Clontech), DsRed2 (“RFP”, Clontech), EBFP, EBFP2, ECFP, EGFP (weak dimer, Clontech), Emerald (weak dimer, Invitrogen), EYFP (weak dimer, Clontech), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRedl, J-Red, Katusha, Kusabira Orange (monomer, MBL), mCFP, mCherry, mCitrine, Midoriishi Cyan (dimer, MBL), mKate (TagFP635, monomer, Evrogen), mKeima-Red (monomer, MBL), mKO, mOrange, mPlum, mRaspberry, mRFP1 (monomer, Tsien lab), mStrawberry, mTFP1, mTurquoise2, P3 (phycobilisome complex), Peridinin Chlorophyll (PerCP), R-phycoerythrin(RPE), T-Sapphire, TagCFP (dimer, Evrogen), TagGFP (dimer, Evrogen), TagRFP (dimer, Evrogen), TagYFP (dimer, Evrogen), tdTomato (tandem dimer), Topaz, TurboFP602 (dimer, Evrogen), TurboFP635 (dimer, Evrogen), TurboGFP (dimer, Evrogen), TurboRFP (dimer, Evrogen), TurboYFP (dimer, Evrogen), Venus, Wild Type GFP, YPet, ZsGreenl (tetramer, Clontech), ZsYellowl (tetramer, Clontech).

In another embodiment, the drug D can be polyalkylene glycols that are used for extending the half-life of the cell-binding antibody, or antibody-like protein molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 40 kDa; two branches each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965, 566; 5,969,040; 5,981,709; 6,011,042; 6,042,822; 6,113,906; 6,127,355; 6,132,713; 6,177,087, and 6,180,095.

In yet another embodiment, D is more preferably a potent cytotoxic agent, selected from a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (e.g., dimers of pyrrolobenzodiazepine (PBD), tomaymycin, anthramycin, indolinobenzodiazepines, imidazobenzothiadiazepines, or oxazolidinobenzo-diazepines) and their analogs, a calicheamicin and the enediyne antibiotic and their analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT) inhibitor, an immunotoxin, and stereoisomers, isosteres, analogs, or derivatives above thereof.

Tubulysin and its analogs are well known in the art and can be isolated from natural sources according to known methods or prepared synthetically according to known methods (e. g. Balasubramanian, R., et al. J. Med. Chem., 2009, 52, 238-40; Wipf, P., et al. Org. Lett., 2004, 6, 4057-60; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; Patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat. Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules through process of the present patent application are described in the patent application of PCT/IB2012/053554.

Tubulysin analog having the following formula (IV):

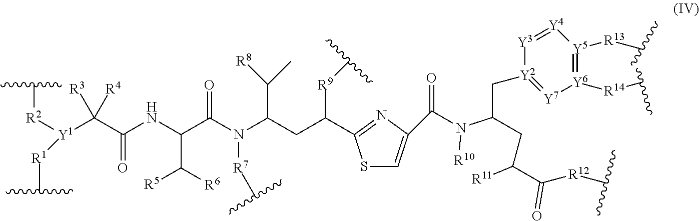

or a pharmaceutically acceptable salt, hydrates, or hydrated salt; or a polymorphic crystalline structure; or an optical isomer, racemate, diastereomer or enantiomer thereof,
wherein [symbol] is a linkage site that links to L₁ and/or L₂ independently;
wherein R¹, R², R³, and R⁴ are independently H, C₁-C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹R², R¹R³, R²R³, R³R⁴, R⁵R⁶, R¹¹R¹² or R¹³R¹⁴ form a 3˜7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R⁸, R¹⁰ and R¹¹ are independently H, or C₁-C₄ alkyl or heteroalkyl;
wherein R⁷ is independently H, R¹⁴, —R¹⁴C(═O)X¹R¹⁵; or —R¹⁴X¹R⁵; X¹ is O, S, S—S, NH, CH₂ or NR¹⁴;
wherein R⁹ is selected from H, OH, —O—, ═O, —OR¹⁴, —OC(═O)R¹⁴, —OC(═O)NHR¹⁴—, —OC(═O) R¹⁴SSR¹⁵—, OP(═O)(OR¹⁴)—, —OC(═O)NR¹⁴R¹⁵, OP(═O)(OR¹⁴), or OR¹⁴OP(═O)(OR¹⁵);
wherein R¹¹ is independently H, R¹⁴, —R¹⁴C(═O)R¹⁶, —R¹⁴X²R¹⁶, —R¹⁴C(═O)X², wherein X² is —O—, —S—, —NH—, —N(R¹⁴)—, —O—R¹⁴—, —S—R¹⁴—, —S(═O)—R¹⁴—, or —NHR¹⁴;
wherein R¹² is R¹⁵, —OH, —SH, —NH₂, NH, NHNH₂, —NH(R¹⁵), —OR¹⁵—, R¹⁵COR¹⁶, —R¹⁵COOR¹⁶, —R¹⁵C(O)NH₂, —R¹⁵C(O)NHR¹⁷, —SR¹⁶, R¹⁵S(═O)R¹⁶, —R¹⁵P(═O)(OR¹⁷)₂, —R¹⁵OP(═O)(OR¹⁷)₂, —CH₂OP(═O)(OR¹⁷)₂, —R¹⁵SO₂R¹⁷, —R¹⁵X²R¹⁶, —R¹⁵C(═O)X², where X² is —O—, OH, SH, —S—, NH₂, —NH—, —N(R¹⁵)—, —O—R¹⁵—, —S—R¹⁵—, —S(═O)—R¹⁵—, CH₂ or —NHR¹⁵—;
R¹³ and R¹⁴ are independently H, O, S, NH, N(R¹⁵), NHNH, —OH, —SH, —NH₂, NH, NHNH₂, —NH(R⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N—(C₄-C₁₂ glycoside); C₁˜C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)₁₋₈ or CO(Aa)₁₋₈(N-terminal or C-terminal 1-8 the same or different amino acids), or polyethyleneoxy unit of formula (OCH₂CH₂)ₚ or (OCH₂CH(CH₃))ₚ, wherein p is an integer from 0 to about 1000, or combination of above groups thereof, X² is O, S, S—S, NH, CH₂, OH, SH, NH₂, CHR¹⁴ or NR¹⁴;
R¹⁵, R¹⁶ and R¹⁷is independently H, C₁-C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, alkylcarbonyl, or Na⁺, K⁺, Cs⁺, Li⁺, Ca²⁺, Mg⁺, Zn²⁺, N⁺(R¹)(R²)(R³) (R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N (R1), thus Y², Y⁴, Y⁵, Y⁶ and Y⁷form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y₄, Y⁵, Y⁶ and Y⁷ form aromatic ring of benzene, pyridine, pyridazine, pyrimidine, pyrazine, triazine, tetrazine, pentazine;

Examples of the structures of the tubulysin analogs are shown below:

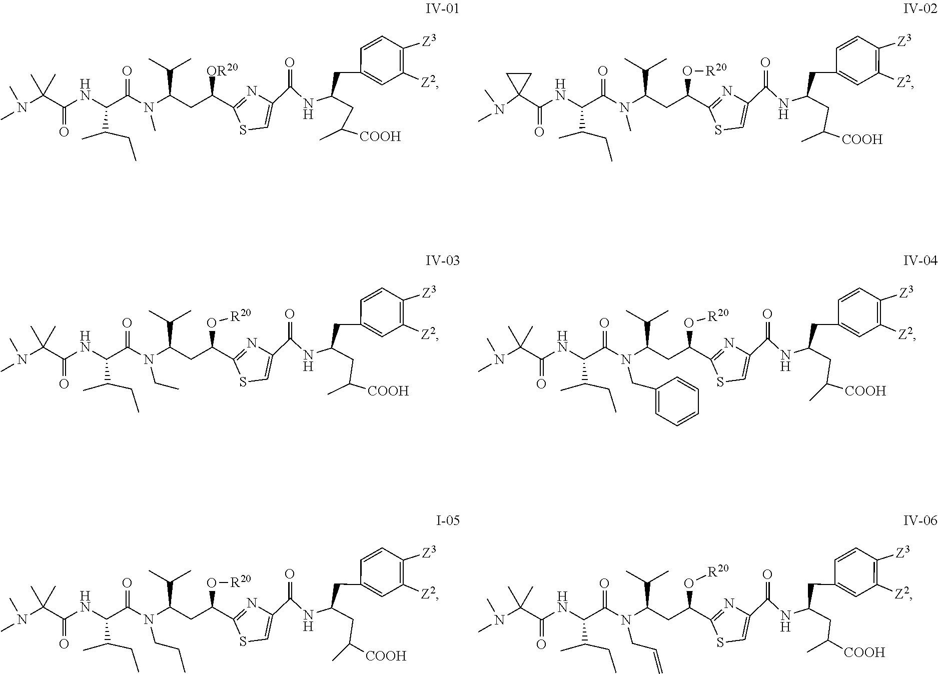

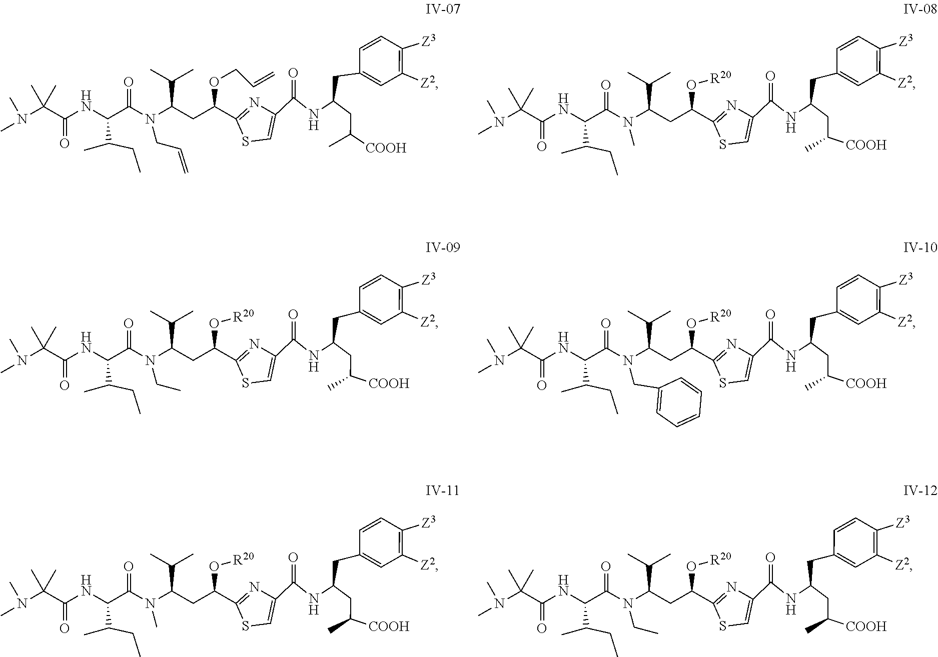

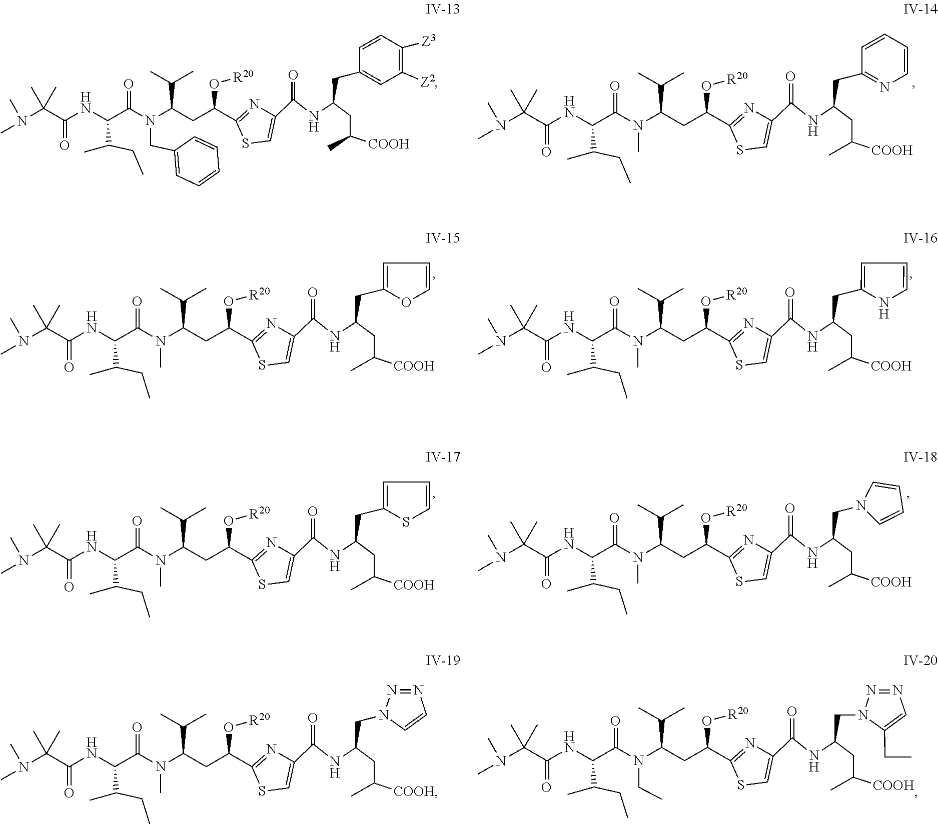

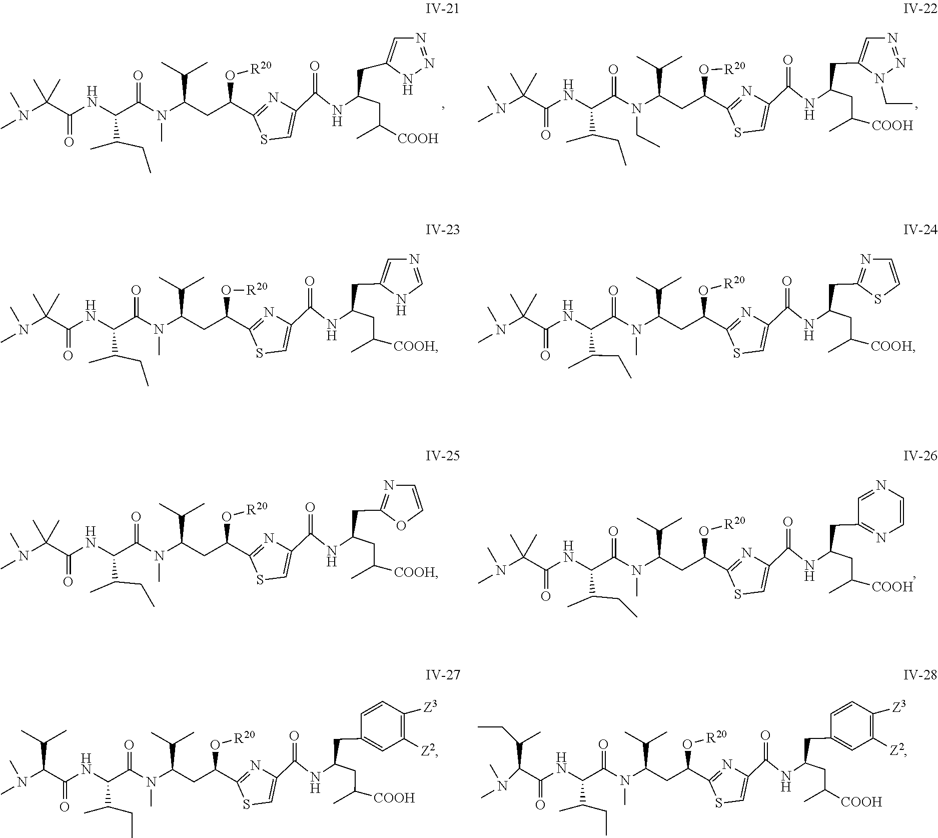

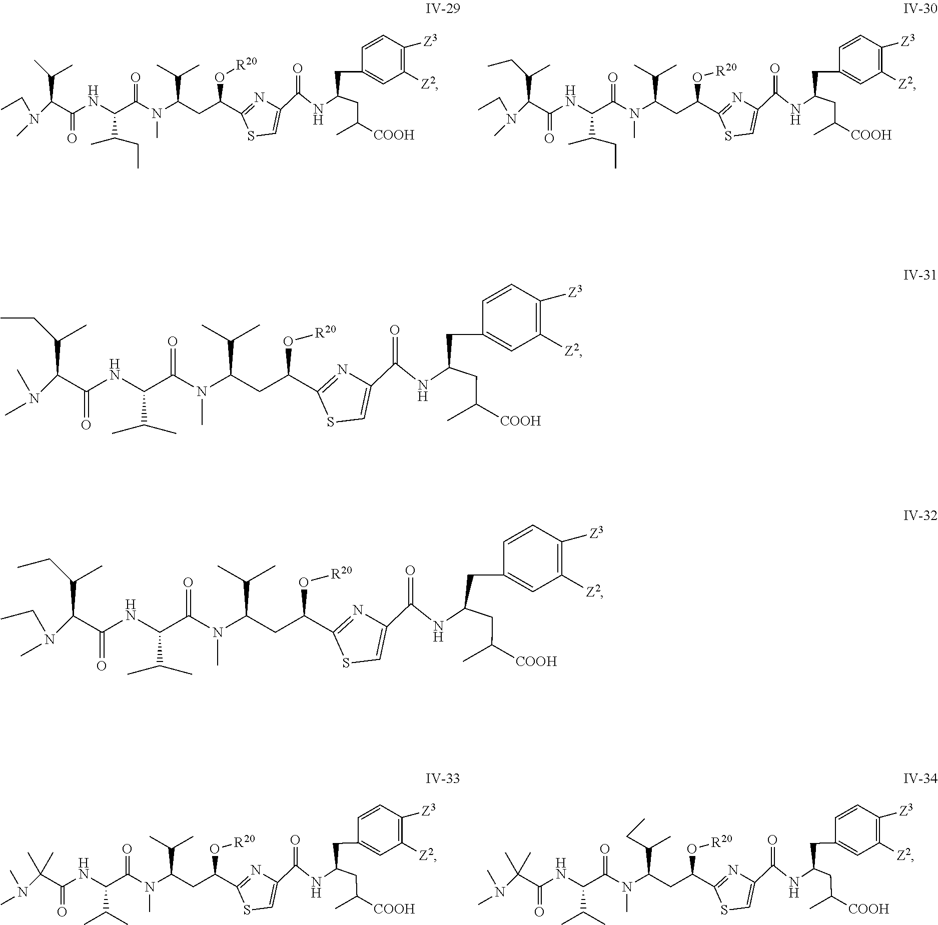

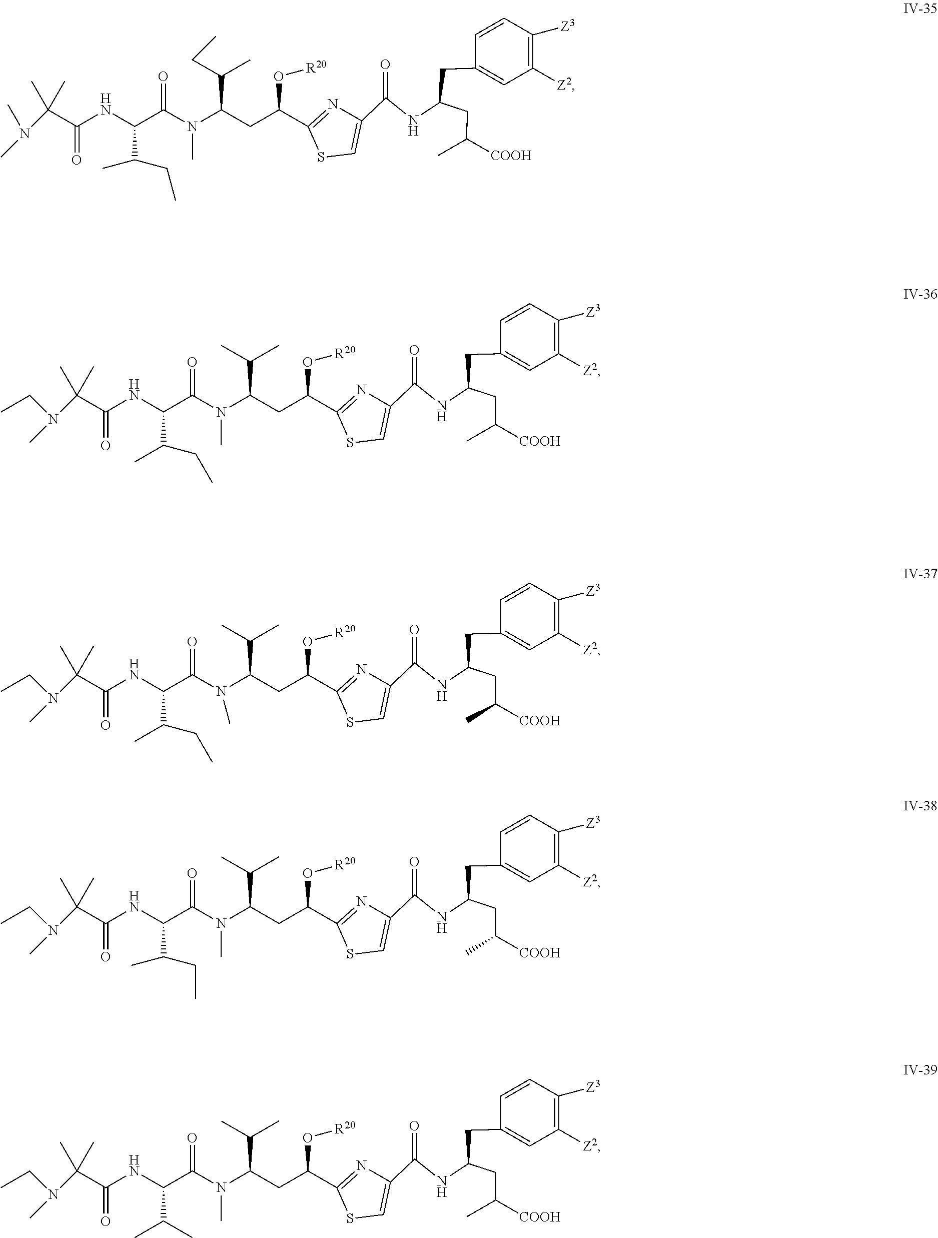

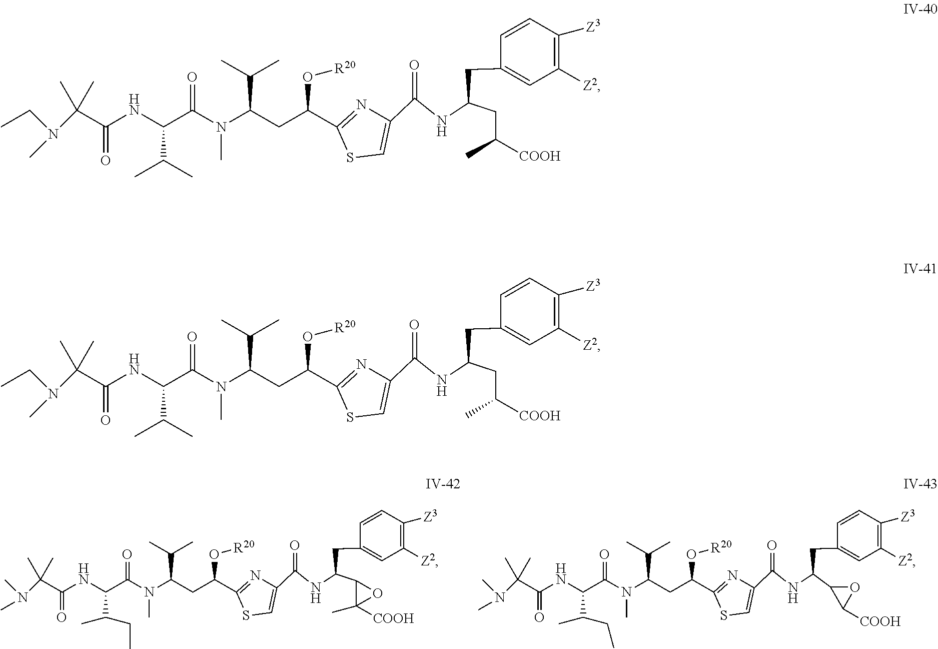

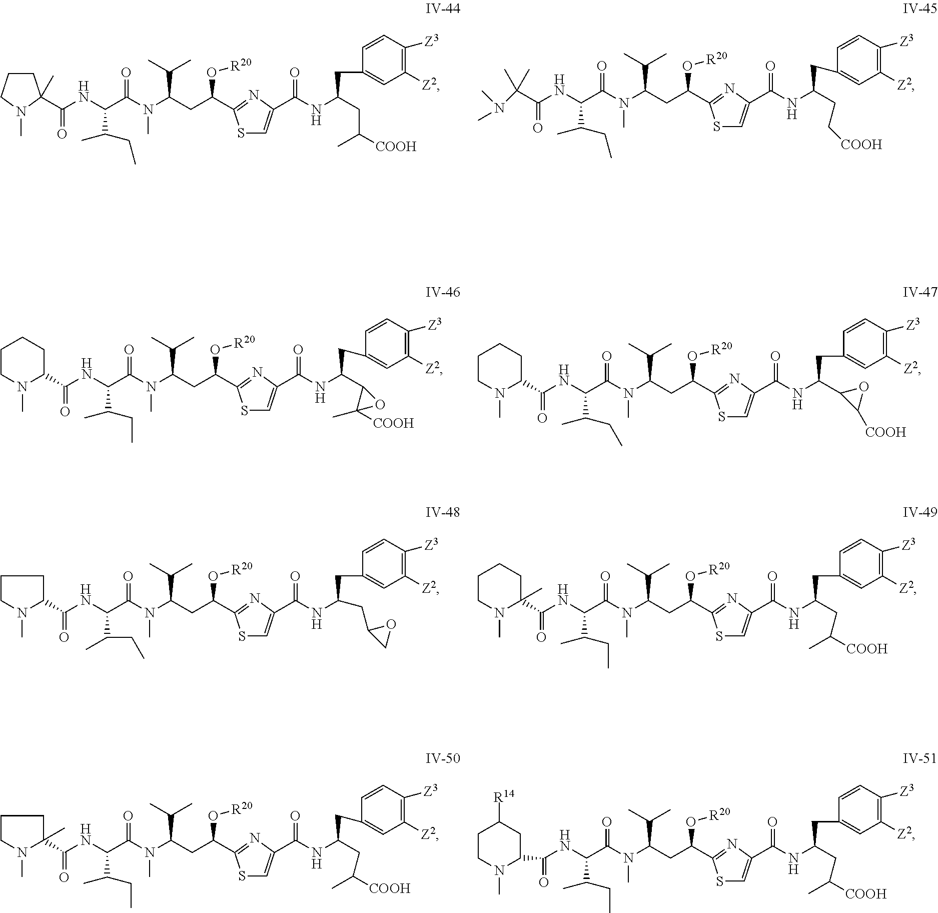

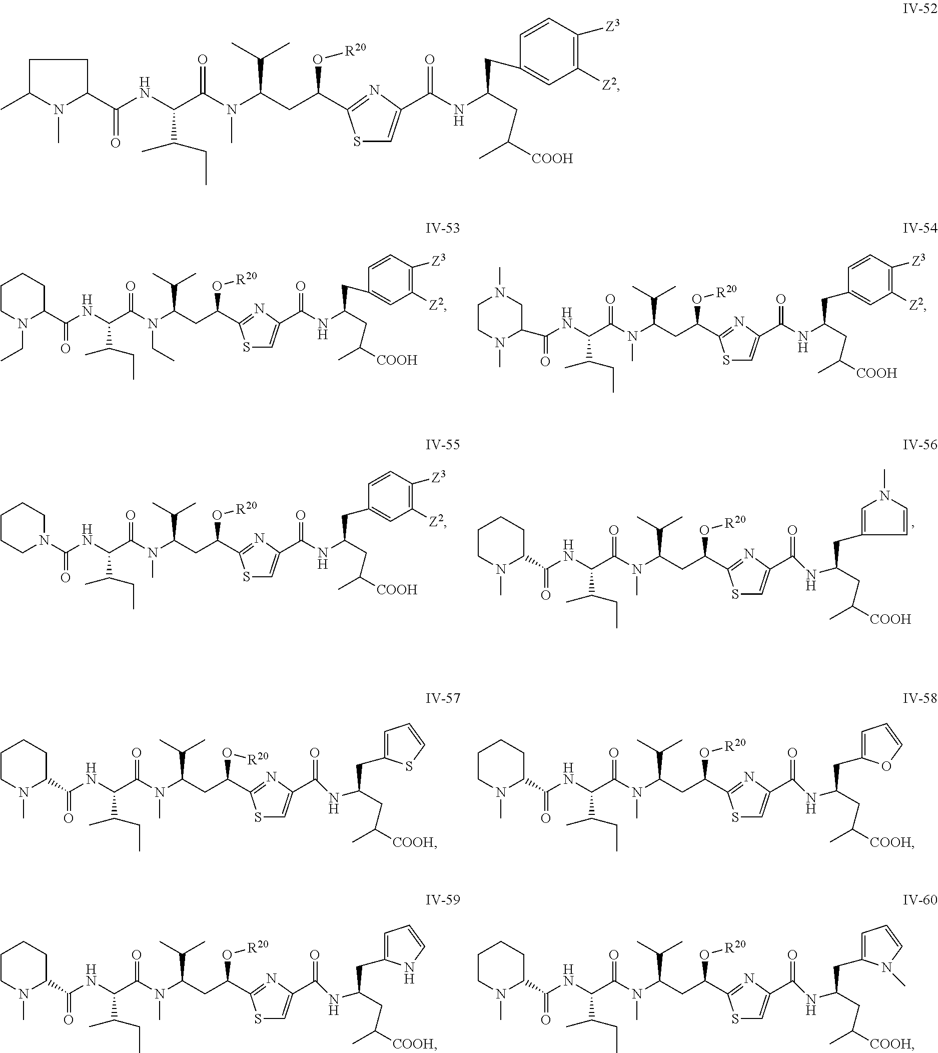

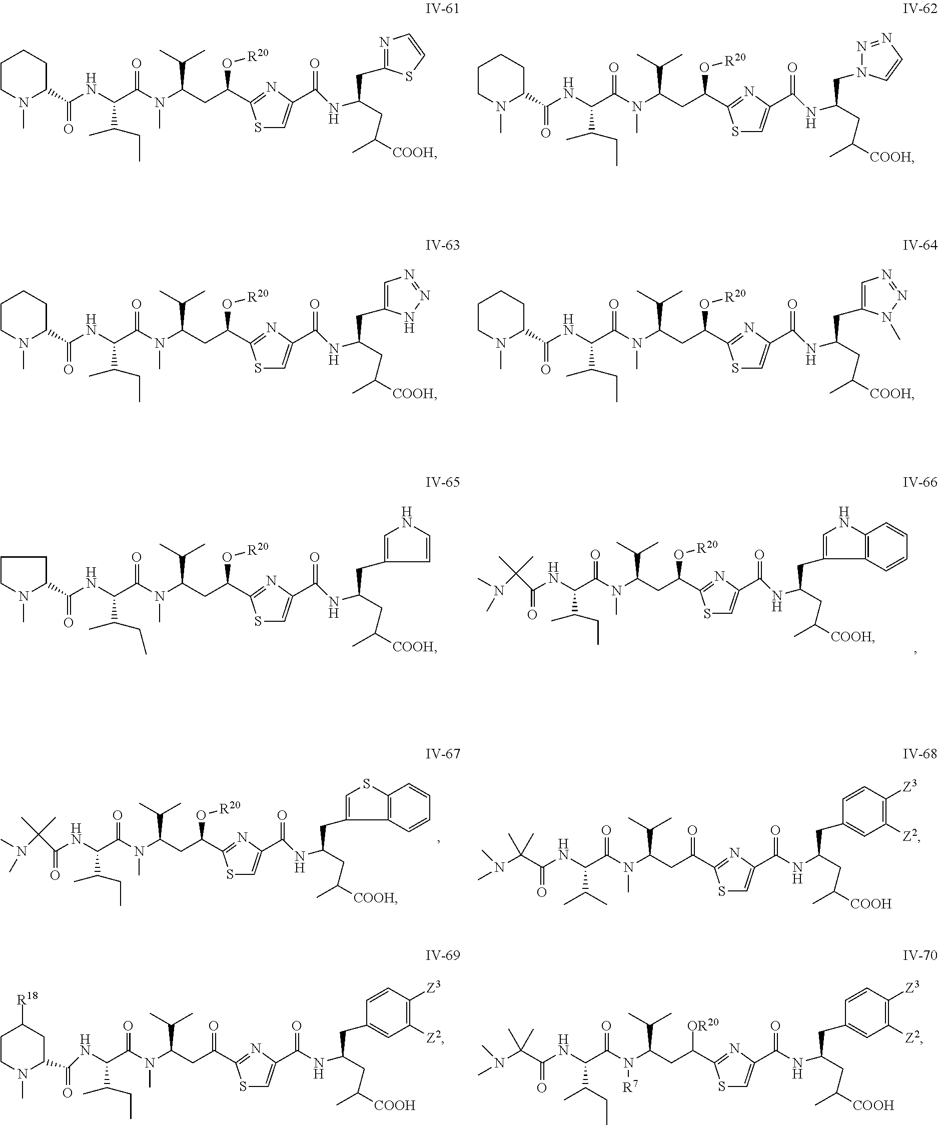

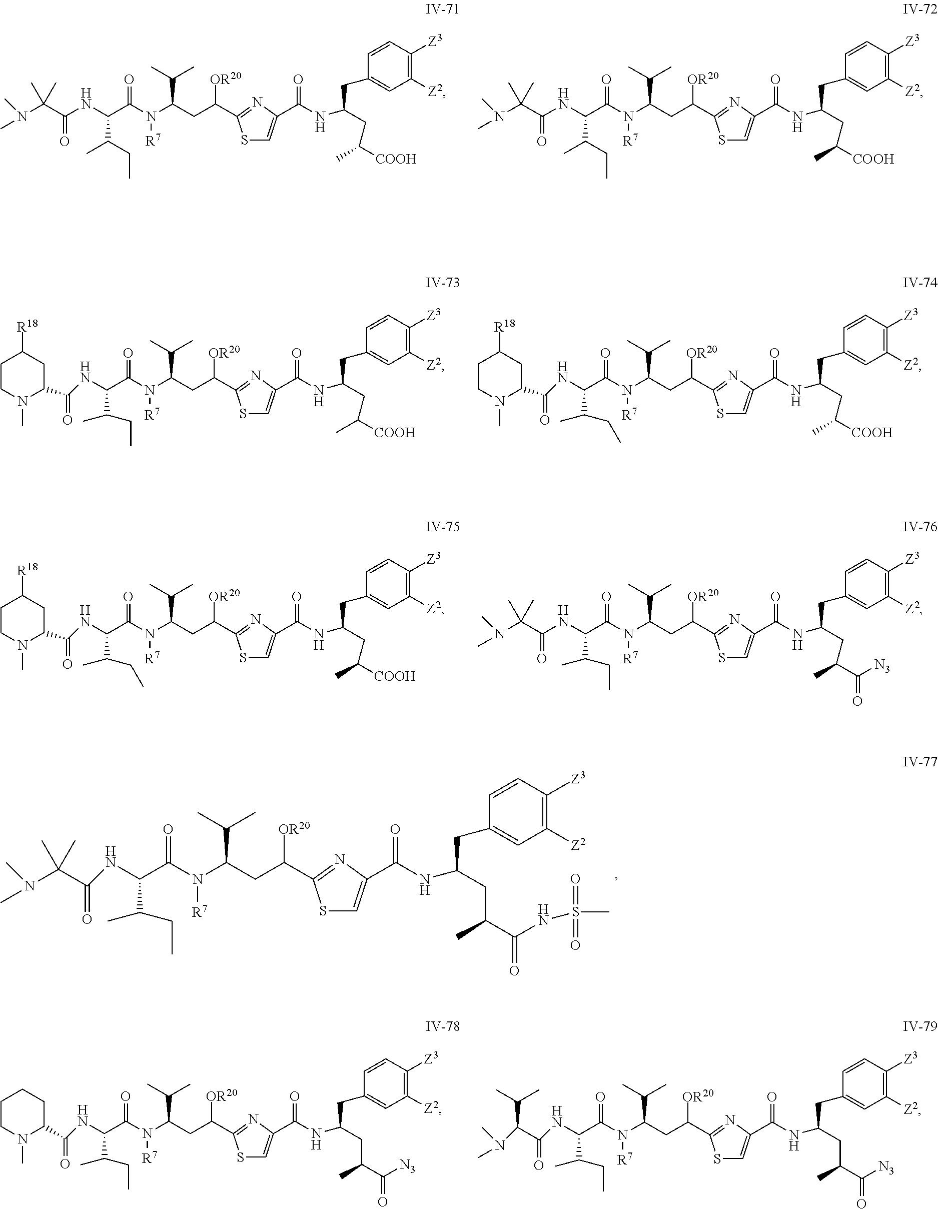

wherein R²⁰ is H; C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); or 1-8 carbon atoms of carboxylate, esters, ether, or amide; or 1˜8 amino acids; or polyethyleneoxy unit of formula (OCH₂CH₂)ₚ or (OCH₂CH(CH₃))ₚ, wherein p is an integer from 0 to about 1000; or R₂₀ is absent and the oxygene forms a ketone, or combination above thereof, Z³ and Z³ are independently H, OH, NH₂, O, NH, COOH, COO, C(O), C(O), C(O)NH, C(O)NH₂, R¹⁸, OCH₂OP(O)(OR¹⁸)₂, OC(O)OP(O)(OR¹⁸)₂, OPO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁹is H, OH, NH₂, OSO₂(OR¹⁸), XCH₂OP(O)(OR¹⁸)₂, XPO(OR¹⁸)₂, XC(O)OP(O)(OR^{1′})₂, XC(O)R¹⁸, XC(O)NHR¹⁸, C₁˜C₈ alkyl or carboylate; C₂˜Cₛ alkenyl, alkynyl, alkylcycloalkyl, heterocycloalkyl; C₃˜Cₛ aryl or alkylcarbonyl; or pharmaceutical salts; X isO, S, NH, NHNH, or CH₂; R⁷ is defined the same above; wherein the linkage sites, “ [symbol] ” in formula IV-01-IV-79 are the same indication according to formula (IV).

Calicheamicins and their related enediyne antibiotics that are described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. Exemplary enediynes include, but are not limited to, calicheamicin, esperamicin, uncialamicin, dynemicin, and their derivatives. The structure of calicheamicins is preferred the following formula:

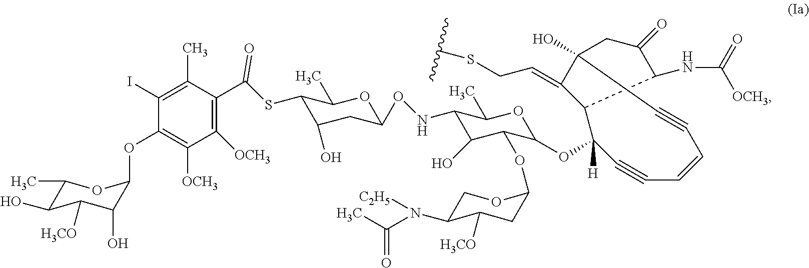 [SMILES: *SC/C=C1\[C@@H]2C(NC(=O)OC)C(=O)C[C@@]1(O)C#C/C=C\C#C[C@@H]2OC1OC(C)C(NOC2CC(O)C(SC(=O)c3c(C)c(I)c(OC4OC(C)C(O)C(OC)C4O)c(OC)c3OC)C(C)O2)C(O)C1OC1CC(OC)C(N(CC)C(C)=O)CO1]

or a isotope of a chemical element, or a pharmaceutically acceptable salt, hydrates, or hydrated salt; or a polymorphic crystalline structure; or an optical isomer, racemate, diastereomer or enantiomer thereof,
wherein [symbol] the site linked to L₁ or L₂;
Geldanamycins are enzoquinone ansamycin antibiotic that bind to Hsp90 (Heat Shock Protein 90) and have been used antitumor drugs. Exemplary geldanamycins include, but are not limited to, 17-AAG (17-N-Allylamino-17-Demethoxygeldanamycin) and 17-DMAG (17-Dimethylaminoethylamino-17-demethoxygeldanamycin).

Maytansines or their derivatives maytansinoids inhibit cell proliferation by inhibiting the microtubules formation during mitosis through inhibition of polymerization of tubulin. See Remillard et al., Science 189:1002-1005 (1975). Exemplary maytansines and maytansinoids include, but are not limited to, mertansines (DM1, DM4), maytansinol and its derivatives as well as ansamitocin. Maytansinoids are described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333.410; 6,441,163; 6,716,821, 7,276,497, 7,301,019, 7,303,749, 7,368,565, 7,411,063, 7,851, 432, and 8,163,888. The structure of maytansinoids is preferred the following formula:

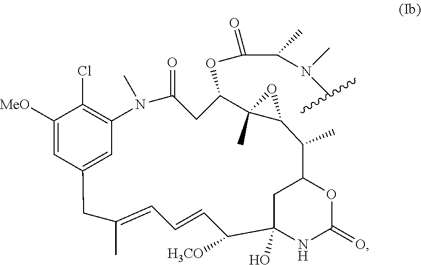

wherein [symbol] is the site linked to L₁ or L₂.

A camptothecin (CPTs) and its derivatives, which are topoisomerase inhibitors to prevent DNA re-ligation and therefore to causes DNA damage resulting in apoptosis, are described in: Shang, X. F. et al, Med Res Rev. 2018, 38(3):775-828; Botella, P. and Rivero-Buceta, E. J Control Release. 2017, 247: 28-54; Martino, E. et al, Bioorg Med Chem Lett. 2017, 27(4):701-707; Lu, A., et al, Acta Pharmacol Sin 2007, 28(2): 307-314. It includes SN-38, Topotecan, Irinotecan (CPT-11), Silatecan (DB-67, AR-67), Cositecan (BNP-1350), Etirinotecan, Exatecan, Lurtotecan, Gimatecan (ST1481), Belotecan (CKD-602), Rubitecan and several others (Shang, X. F. et al, Med Res Rev. 2018, 38(3):775-828). So far three CPT analogues, topotecan, irinotecan, and belotecan have been approved and are used in cancer chemotherapy (Palakurthi, S., Expert Opin Drug Deliv. 2015; 12(12):1911-21; Shang, X. F. et al, Med Res Rev. 2018, 38(3):775-828) and both SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21(17): 3870-8; Cardillo, T. M., et al, Bioconjug Chem. 2015, 26(5): 919-31; Ogitani, Y. et al, Bioorg Med Chem Lett. 2016, 26(20): 5069-5072; Takegawa, N. et al, Int J Cancer. 2017 Oct. 15; 141(8):1682-1689. US patents 7, 591, 994; 7, 999,083, 8,080, 250, 8, 268, 317; US patent applications 20130090458, 20140099258, 20150297748, 20160279259).

The structure of Camptothecin (CPT) is illustrated below formula:

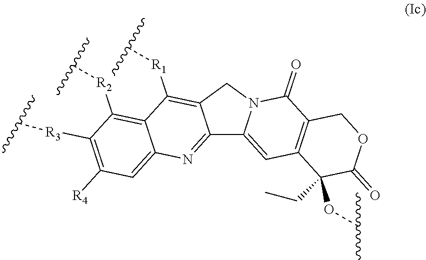

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁˜C₈ alkyl; O—C₁˜C₈ alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate; R₃ is H, OH, NH₂, C₁˜C₈ alkyl; O—C₁˜C₈ alkyl; NH—C₁˜C₈ alkyl; C₂-C₈ of heteroalkyl, alkylcycloalkyl, heterocycloalkyl; or 2-8 carbon atoms of esters, ether, amide, carbonate, urea, or carbamate; or R₁R₂, R₂R₃ and R₃R₄ independently form a 5˜7 membered carbocyclic, heterocyclic, heterocycloalkyl, aromatic or heteroaromatic ring system; [symbol] is the site in the molecule that can be linked to Lᵢ or L₂.

The structures of camptothecins are preferred the following formula:

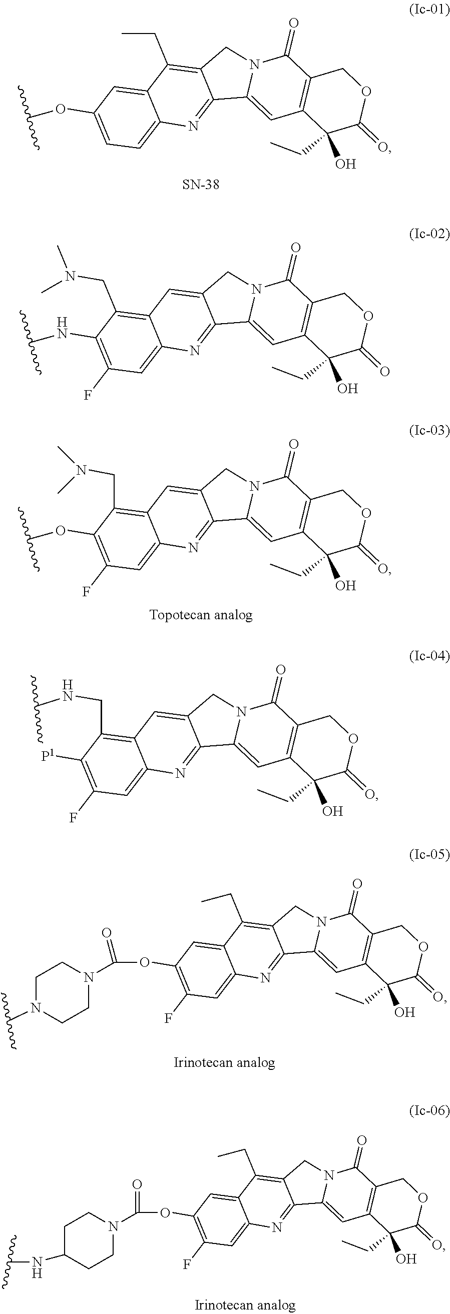

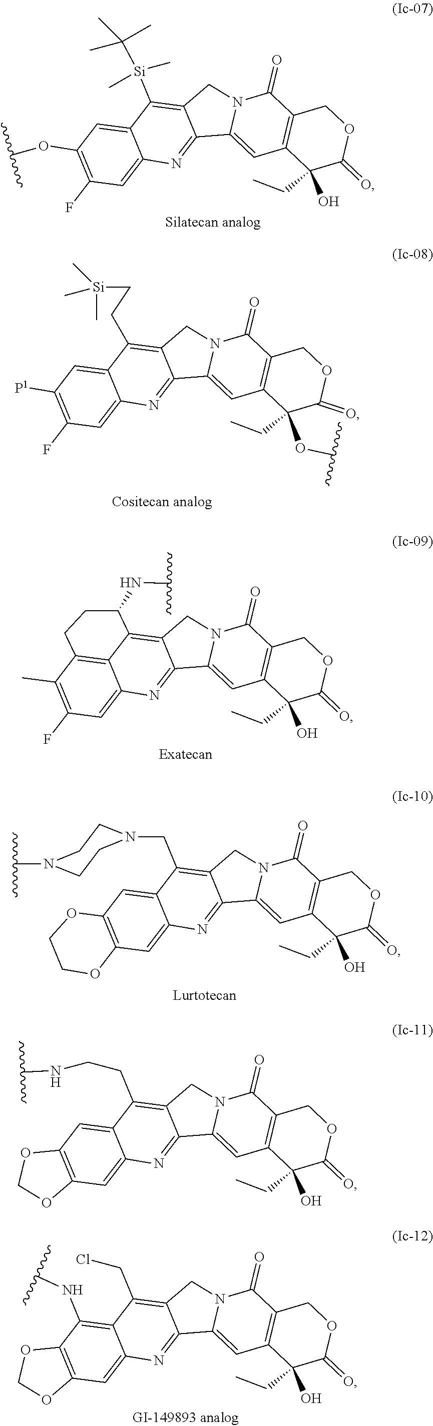

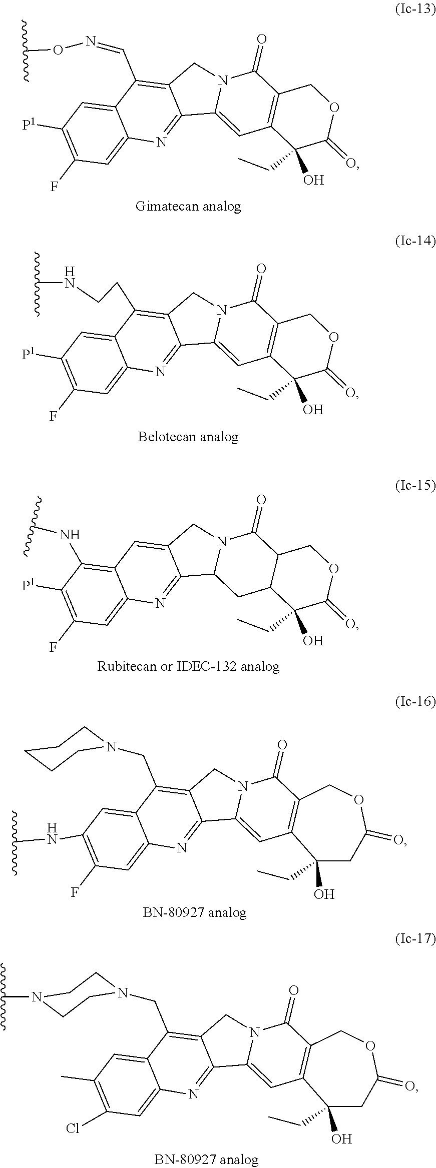

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the site linked to L₁ or L₂; P¹ is H, OH, NH₂, COOH, C(O)NH₂, OCH₂OP(O)(OR¹⁸)₂, OC(O)OP(O(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸).

Combretastatins are natural phenols with vascular disruption properties in tumors. Exemplary combretastatins and their derivatives include, but are not limited to, combretastatin A-4 (CA-4), CA4-βGals, CA-4PD, CA4-NPs and ombrabulin.

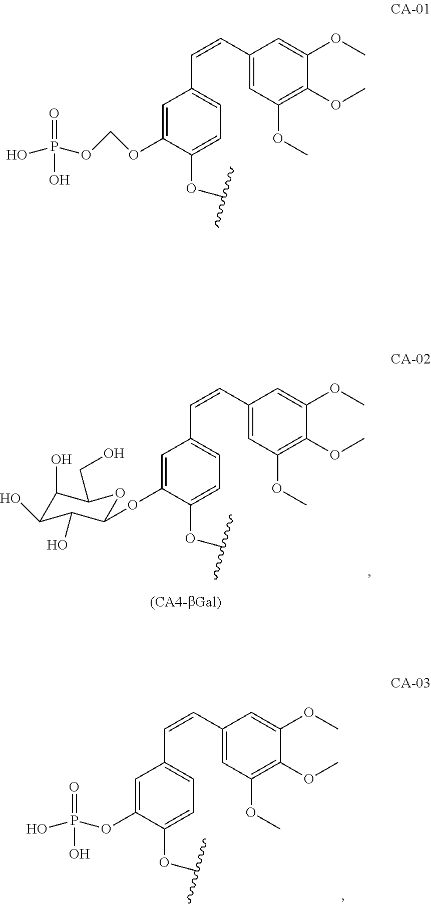 [SMILES: *Oc1ccc(/C=C\c2cc(OC)c(OC)c(OC)c2)cc1OCOP(=O)(O)O ; *Oc1ccc(/C=C\c2cc(OC)c(OC)c(OC)c2)cc1OC1OC(CO)C(O)C(O)C1O ; *Oc1ccc(/C=C\c2cc(OC)c(OC)c(OC)c2)cc1OP(=O)(O)O]

Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Pat. Nos. 5,475,011 5,728, 849, 5,811,452; 6,340,701; 6,372,738; 6,391,913, 6,436,931; 6,589,979; 6,596,757; 6,706,708; 7,008,942; 7,186,851; 7,217,819; 7,276,499; 7,598,290; and 7,667,054. The structures of taxanes are preferred the following formula:

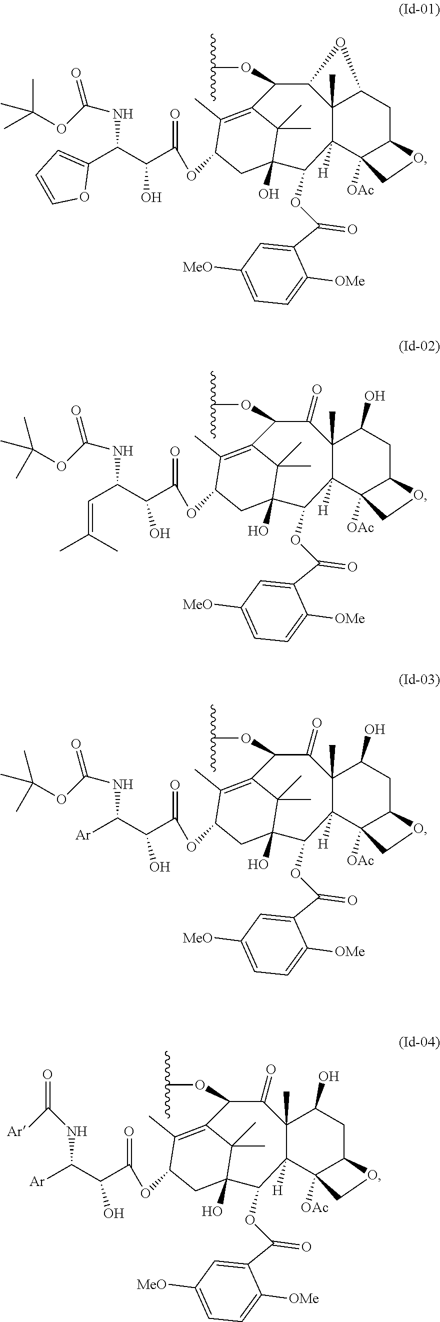

wherein [symbol] is the site linked to Lᵢ or L₂; Ar and Ar′ are independently aryl or heteroaryl.

Anthracyclines are mammalian DNA topoisomerases II inhibitors that are able to stabilize enzyme-DNA complexes wherein DNA strands are cut and covalently linked to the antibody-like protein. These anticancer agents maintain a prominent role in treating many forms of solid tumors and acute leukemias during the last several decades. However, anthracyclines cause cardiovascular morbidity and mortality (Sagi, J. C., et al, Pharmacogenomics. 2016, 17(9), 1075-87; McGowan, J. V., et al, Cardiovasc Drugs Ther. 2017, 31(1), 63-75). Thus, to enhance specific activity of such molecules while reducing the cardiotoxicity, reasearchers actively are using the conjugation of anthracyclines to a cell-binding antibody, or antibody-like protein molecule as a general approach for improving the therapeutic index of these drugs, (Mollaev, M. et al, Int J Pharm. 2018 Dec. 29. pii: S0378-5173(18) 30991-8; Rossin, R., et al, Bioconjug Chem. 2016, 27(7):1697-706; Dal Corso, A., et al, J Control Release. 2017, 264:211-218). Exemplary anthracyclines include, but are not limited to, daunorubicin, doxorubicin (i.e., adriamycin), epirubicin, idarubicin, valrubicin, and mitoxantrone. The structures of anthracyclines used for the present application are preferred the following formula:

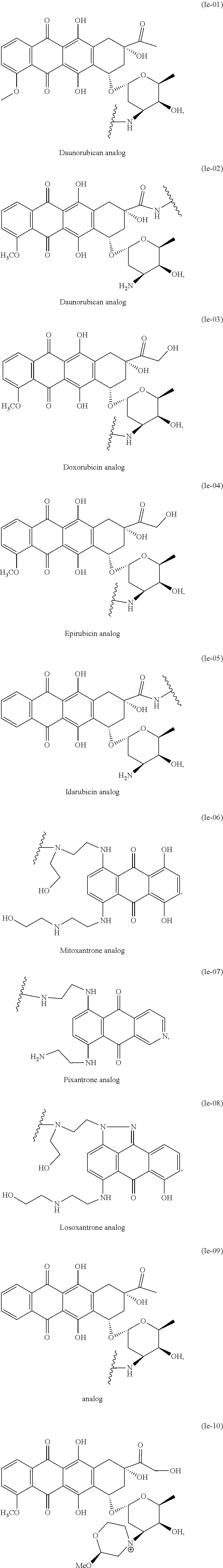

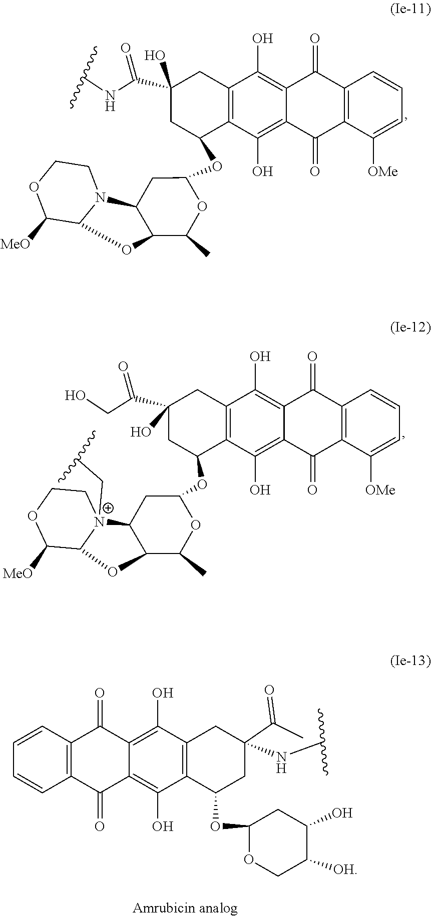

wherein [symbol] is the site that links to L₁ or L₂.

Vinca alkaloids are a set of anti-mitotic and anti-microtubule alkaloid agents that work by inhibiting the ability of cancer cells to divide. Vinca alkaloids include vinblastine, vincristine, vindesine, leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine. The structures of vinca alkaloids are preferred vinblastine, vincristine having the following formula:

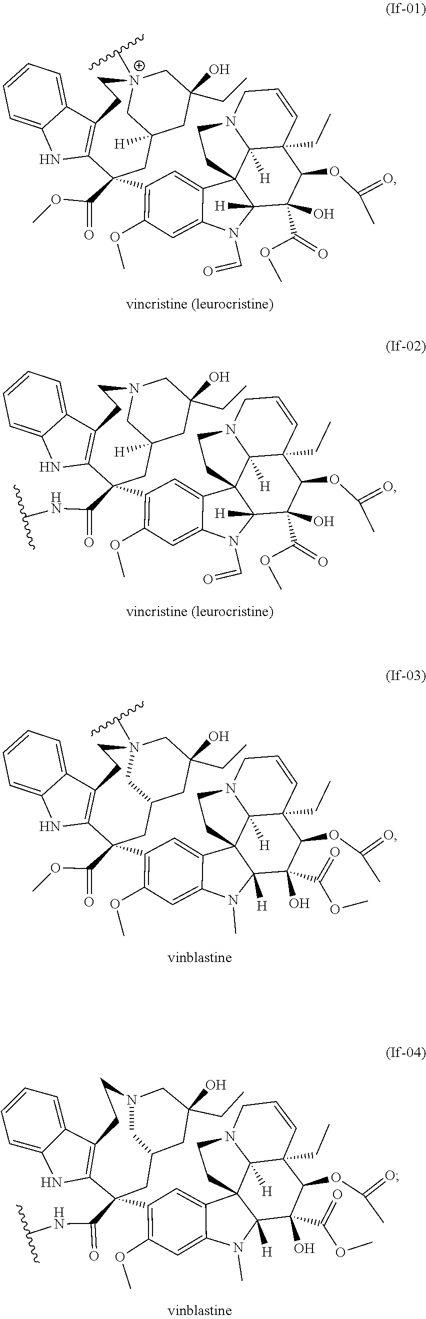

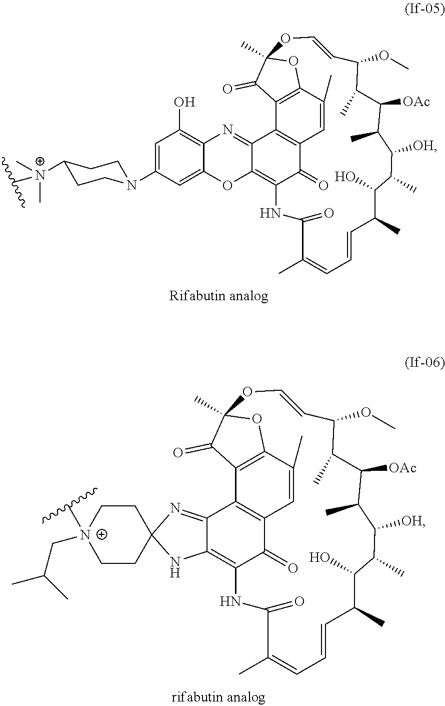

wherein [symbol] is the site linked to L₁ or L₂;
Dolastatins and their peptidic analogs and derivatives, auristatins, are highly potent antimitotic agents that have been shown to have anticancer and antifungal activity. See, e.g., U.S. Pat. No. 5,663, 149 and Pettit et al., Antimicrob. Agents Chemother. 42:2961-2965, 1998. Exemplary dolastatins and auristatins include, but are not limited to, dolastatin 10, auristatin E (AE), auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), Auristatin F phenylene diamine (AFP) and other novel auristatins. The auristatins are described in Int. J. Oncol. 15: 367-72 (1999); Molecular Cancer Therapeutics, vol. 3, No. 8, pp. 921-32 (2004); U.S application Ser. No. 11/134,826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. The structures of auristatin analogs are preferred the following formula (Ih-01), (Ih-02), (Ih-03), (Ih-04), (Ih-05), (Ih-06), (Ih-07), (Ih-08), (Ih-09), (Ih-10), and (Ih-11):

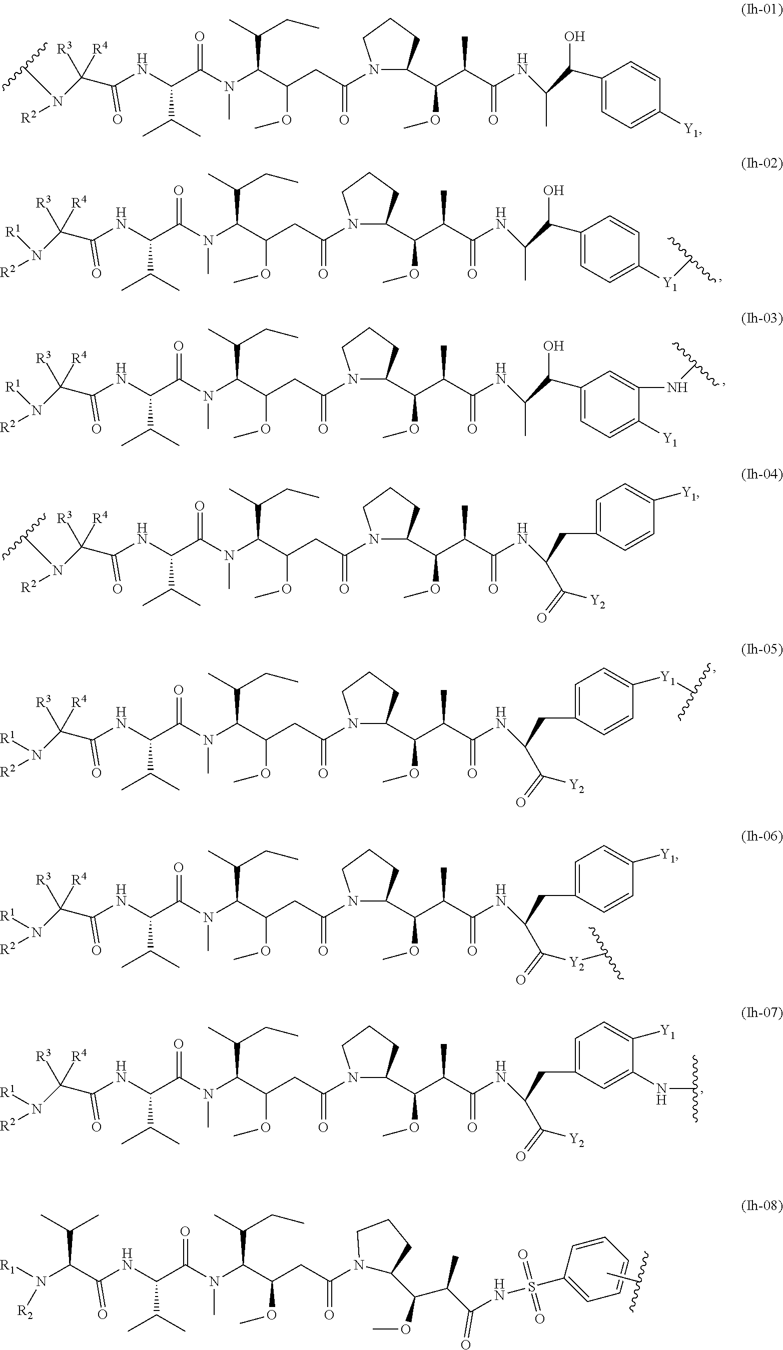

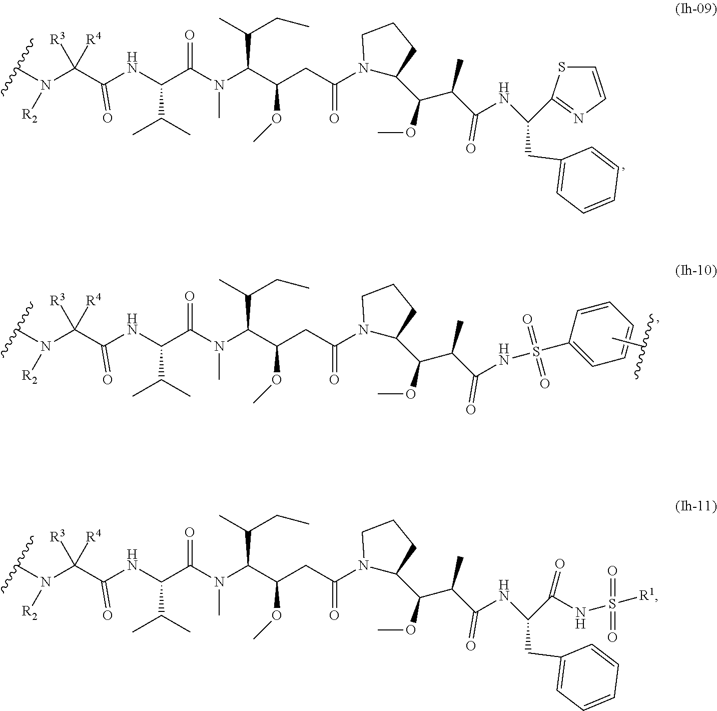

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 1000. The two Rs: R¹R², R₂R₃, R¹R³ or R³R⁴ together can form 3˜8 member cyclic ring of alkyl, aryl, heteroaryl, heteroalkyl, or alkylcycloalkyl group; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR_{I} when not linked to the connecting site “ [symbol] ”; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NI-)ₚCH₂—CH₂NH₂, NH(CH₂CH₂S)ₚCH₂CH₂NH₂, NH(CH₂CH₂NI-)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-5000; R₁, R₂, R₃, R₄, R₅, R₅′, Z₁, Z₂, and n are defined the same above.

Hemiasterlin and its analogues (e.g., HTI-286) bind to the tubulin, disrupt normal microtubule dynamics, and, at stoichiometric amounts, depolymerize microtubules. The structure of maytansinoids is preferred the following formula:

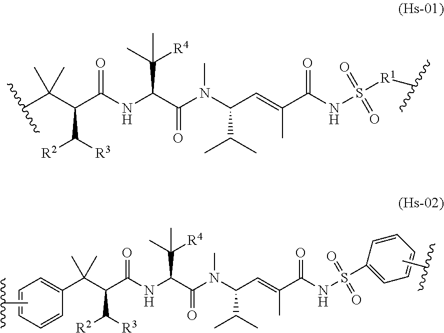

wherein wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; In addition, R²R³ can form 3˜8 member cyclic ring of alkyl, aryl, heteroaryl, heteroalkyl, or alkylcycloalkyl group.

Eribulin which is binding predominantly to a small number of high affinity sites at the plus ends of existing microtubules has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6.; Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). The structure of Eribulin is preferred the following formula, Eb01:

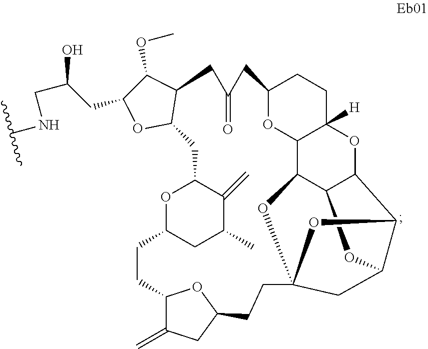 [SMILES: *NC[C@@H](O)C[C@H]1O[C@H]2C[C@H]3O[C@@H](CC[C@@H]4O[C@@H](CC[C@@]56C[C@H]7O[C@@H]8C(O[C@H]9CC[C@H](CC(=O)C[C@@H]2[C@H]1OC)OC9[C@@H]8O5)[C@H]7O6)CC4=C)C[C@@H](C)C3=C]

[symbol] is a linkage site that links to L₁ and/or L₂ independently;

An Inhibitor of nicotinamide phosphoribosyltransferases (NAMPT) can be an interesting ADC payload due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus using ADCs for targeting delivery of NAMPT inhibitors might circumvent the systemic toxicities to achieve much broader therapeutic index. The structures of NAMPT inhibitors are preferred the following formula, NP01, NP02, NP03, NP04, NP05, NP06, NP07, NP08, and NP09:

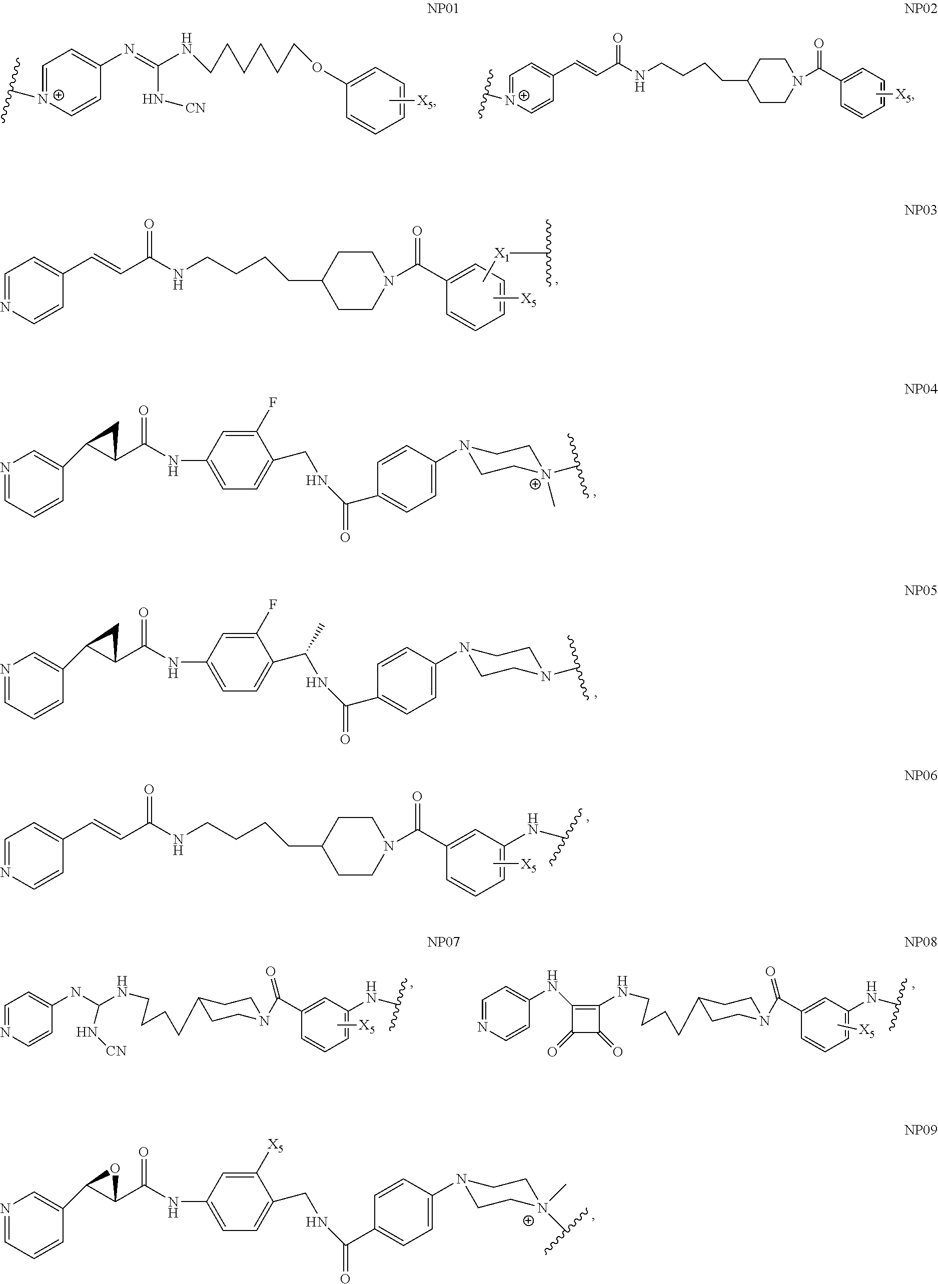

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁.

A benzodiazepine dimer and its analogs: (e. g. a dimer of pyrrolobenzodiazepine (PBD) or (tomaymycin), a dimer of indolinobenzodiazepine (IGN), a dimer of imidazobenzothiadia-zepine, or a dimer of oxazolidinobenzodiazepines) are anti-tumor agents that contain one or more immine functional groups, or their equivalents, that bind to duplex DNA. PBD and IGN molecules are based on the natural product athramycin, and interact with DNA in a sequence-selective manner, with a preference for purine-guanine-purine sequences. The preferred benzodiazepine dimers according to the present invention are exampled in: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109,193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; US patent appl. 20100203007, 20100316656, 20030195196. Examples of the structures of the conjugate of the antibody- benzodiazepine dimers are illustrated below PB01, PB02, PB03, PB04, PB05, PB06, PB07, PB08, PB09, PB10, PB 11, PB12, PB13, PB14, PB15, and PB16.

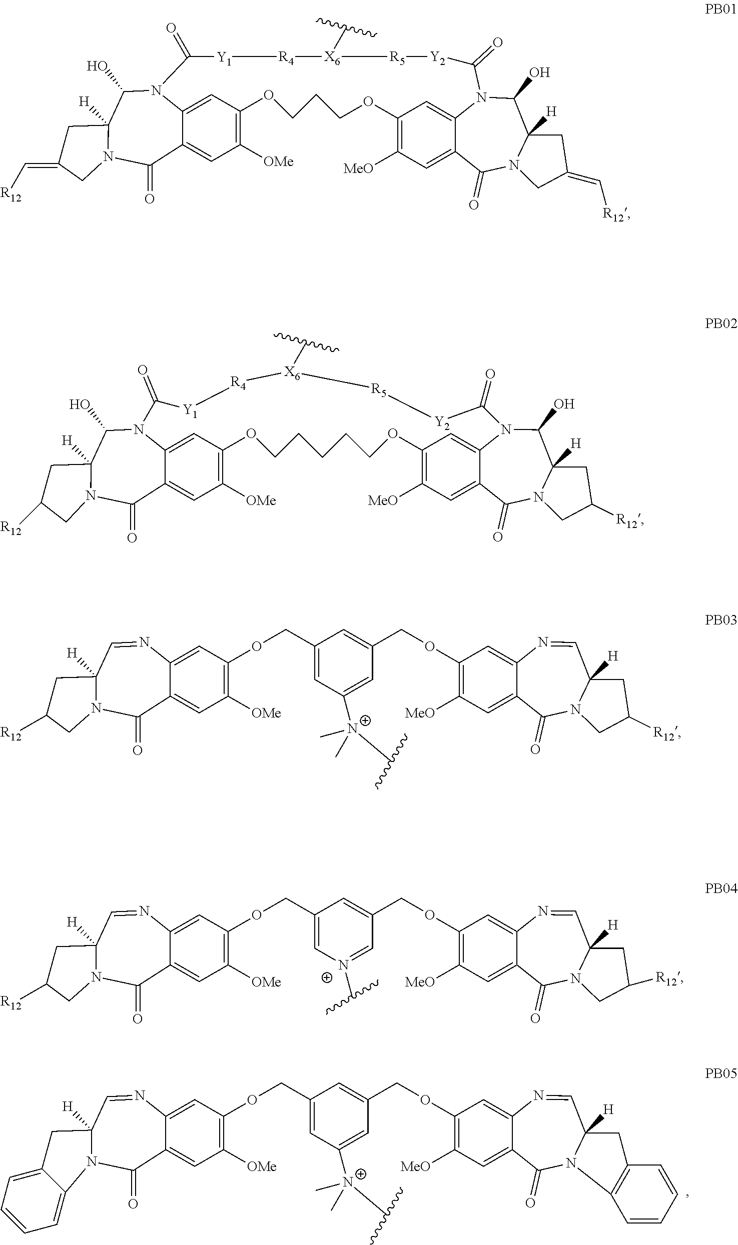

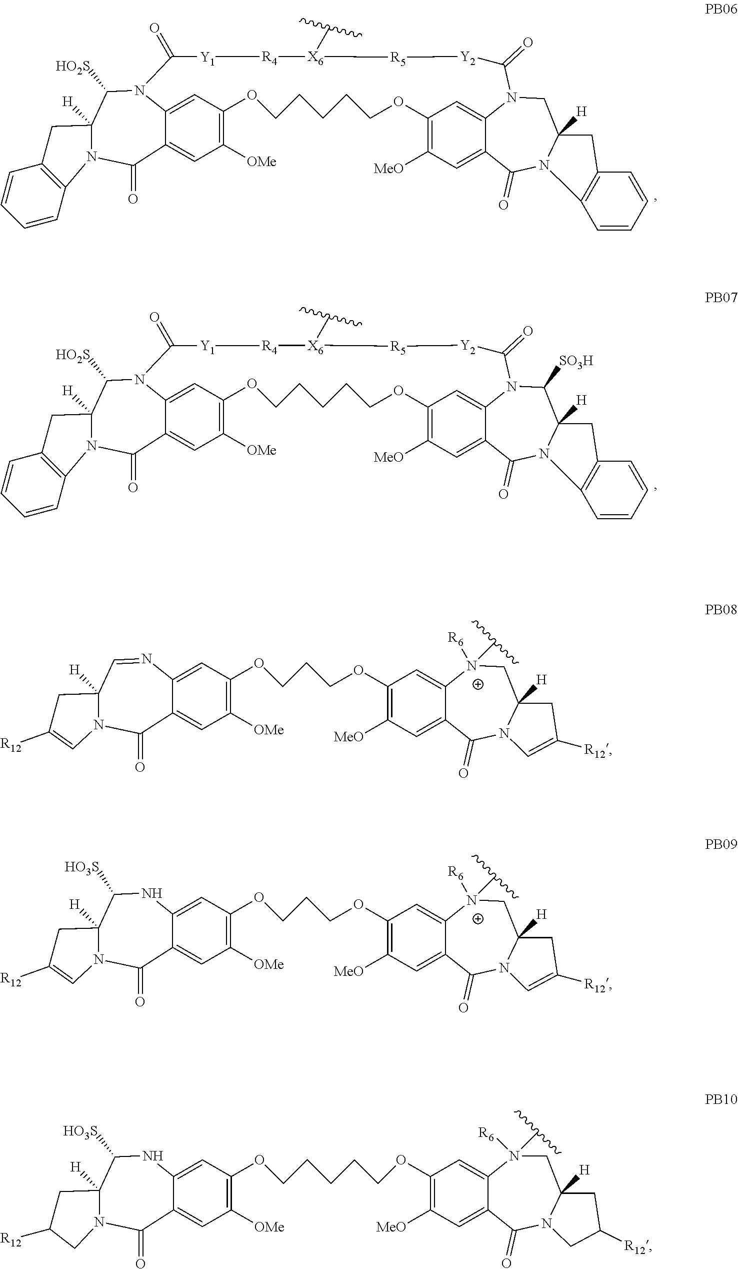

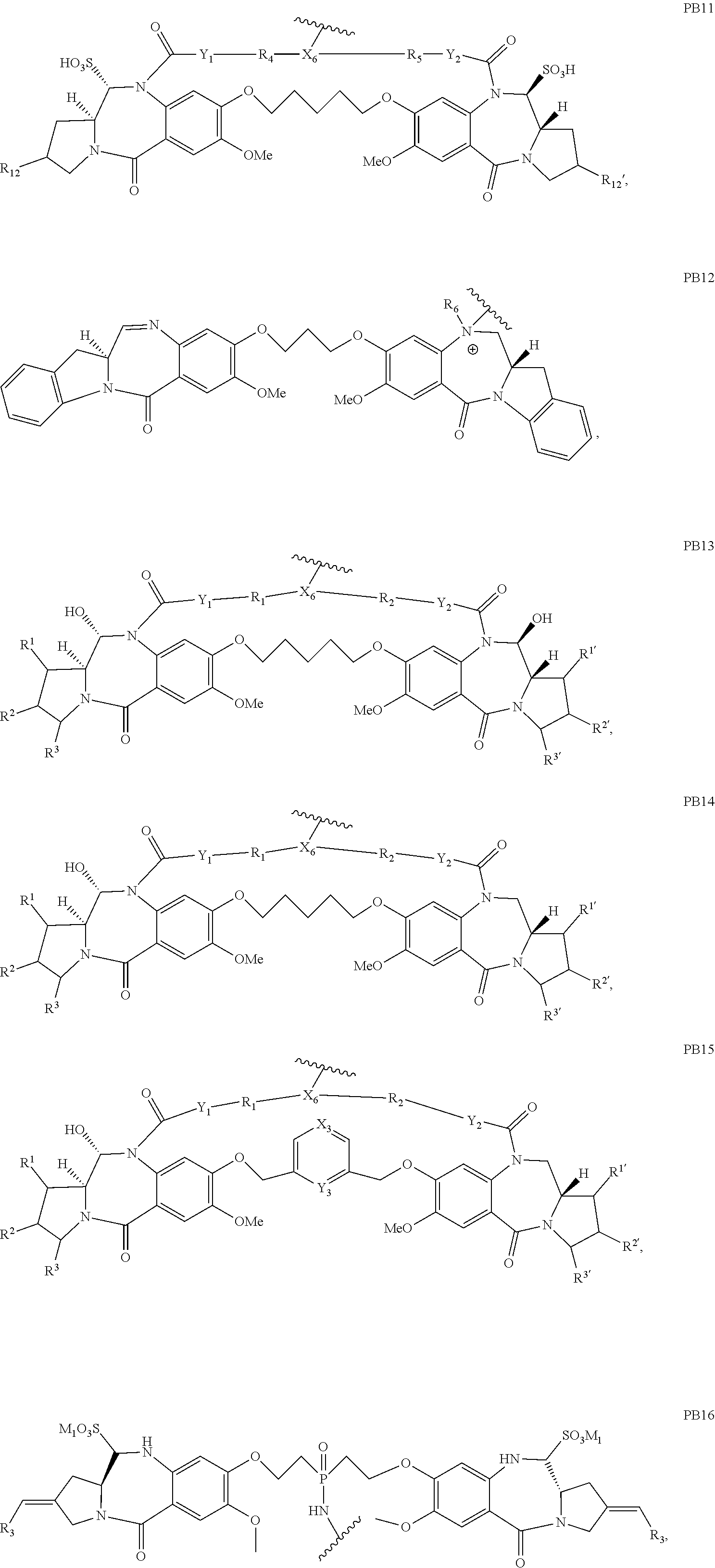

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁, Y₂, Z₁, Z₂, and n are defined the same above; Preferabably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR_{I}; R¹, R², R³, R^{1′}, R^{2′}, and R^{3′} are independently H; F; Cl; ═O; ═S; OH; SH; C₁-C₈ linear or branched alkyl, aryl, alkenyl, heteroaryl, heteroalkyl, alkylcycloalkyl, ester (COOR₅ or —OC(O)R₅), ether (OR₅), amide (CONR₅), carbamate (OCONRs), amines (NHR₅, NR₅R₅′), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000. The two Rs: R¹, R², R²R³, R¹R³, R^{1′}R^{2′}, R^{2′}, R^{3′}, or R^{1′}R^{3′} can independently form 3-8 member cyclic ring of alkyl, aryl, heteroaryl, heteroalkyl, or alkylcycloalkyl group; X₃ and Y₃ are independently N, NH, CH₂ or CR₅, or one of X₃ and Y₃ can be absent; wherein R₁, and R₂ are C₁-C₈ linear or branched alkyl, heteroalkyl; C₃-C₈ aryl, heteroaryl, alkylcycloalkyl, acyloxyl, alkylaryl, alkylaryloxyl, alkylarylamino, alkylarylthiol; or 1-6 the same or different sequence of amino acid/peptides (Ar)r, r=1-6; wherein R₄, R₅, R₅′, R₆, R₁₂ and R_{12′} are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃; X₆ is CH, N, P(O)NH, P(O)NR₁, CHC(O)NH, C₃-C₈ aryl, heteroaryl, alkylcycloalkyl, acyloxyl, alkylaryl, alkylaryloxyl, alkylarylamino, or an Aa (amino acid is preferably selected from Lys, Phe, Asp, Glu, Ser, Thr, His, Cys, Tyr, Trp, Gln, Asn, Arg); “ [symbol] ” is defined the same above.

An CC-1065 analog and doucarmycin analogs are also preferred to be used for a conjugate of the present process invention. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6, 586, 618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837,7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are illustrated below CC01, CC02, CC03, CC04, CC05, CC06 and CC07:

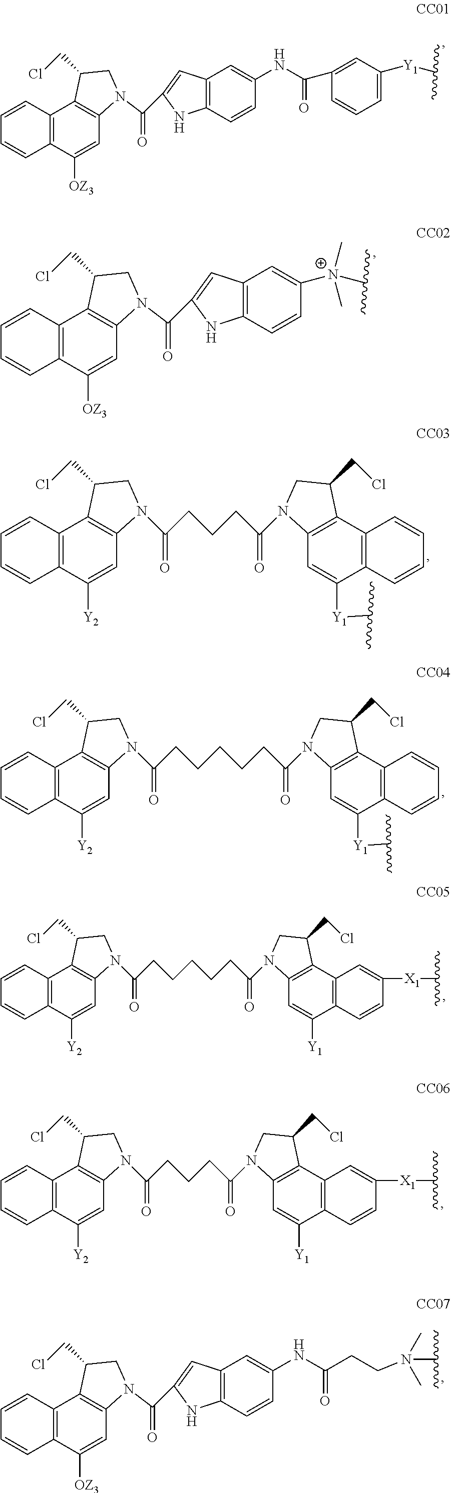

wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above;

An amatoxin and its analogs which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3): 257-76). Amatoxins can be produced from collected Amanita phalloides mushrooms (Yocum, R. R. 1978 Biochemistry 17(18): 3786-9; Zhang, P. et al, 2005, FEMS Microbiol. Lett. 252(2), 223-8), or from fermentation using a basidiomycete (Muraoka, S. and Shinozawa T., 2000 J. Biosci. Bioeng. 89(1): 73-6) or from fermentation using A. fissa (Guo, X. W., et al, 2006 Wei Sheng Wu Xue Bao 46(3): 373-8), or from culturing Galerina fasciculata or Galerina helvoliceps, a strain belonging to the genus (WO/1990/009799, JP 11137291). However, the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M. O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68: 189-96; WO2014/043403, US20150218220, EP 1661584). We have been working on the conjugation of amatoxins for a while. Examples of the structures of the amatoxins used for the present application are preferred the following structures of Am01, Am02, and Am03:

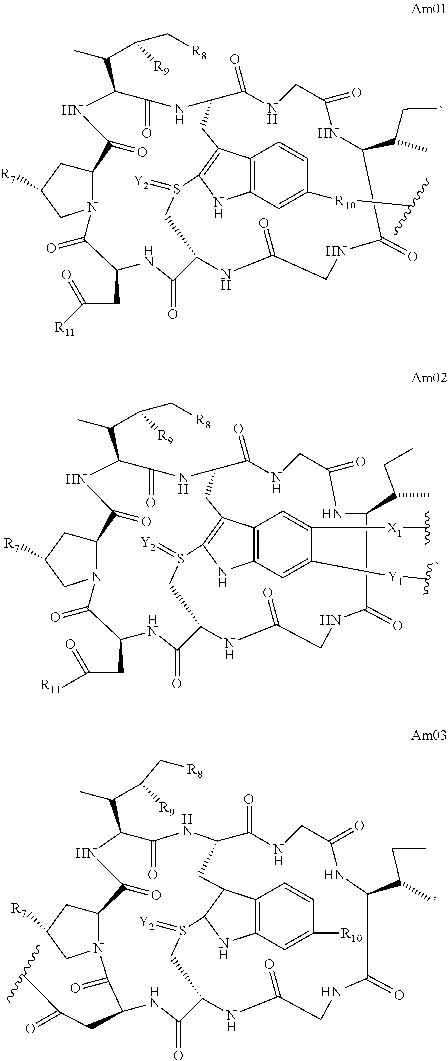

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; Rio is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)P—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa)ᵣ is 1-8 aminoacids; n and mᵢ are independently 1-20; p is 1-5000; R₁, R₂ and Ar, are the same defined through out the application; “ [symbol] ” is defined the same above.

Spliceostatins and pladienolides are anti-tumor compounds which inhibit splicing and interacts with spliceosome, SF3b. Examples of spliceostatins include, but are not limited to, spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)-4-hydroxy-1, 6-dioxaspiro[2.5]oct-5-yl]-3-methylpenta-2, 4-dien-1-y-1}-2, 5-dimethyltetrahydro-2H-pyran-3-yl]amino}-5-oxopent-3-en-2-yl acetate having the core structure:

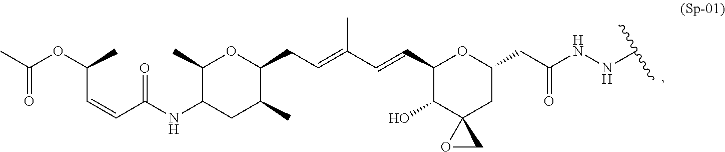 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]

Examples of pladienolides include, but are not limited to, Pladienolide B, Pladienolide D, and E7107.

Protein kinase inhibitors that block the action of an enzyme to add a phosphate (PO₄) group to serine, threonine, or tyrosine amino acids on an antibody-like protein, and can modulate the protein function. The protein kinase inhibitors can be used to treat diseases due to hyperactive protein kinases (including mutant or overexpressed kinases) in cancer or to modulate cell functions to overcome other disease drivers. The structures of protein kinase inhibitors are preferred to selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, Nazartinib (EGF816), having the following formula, PKO1˜PK40:

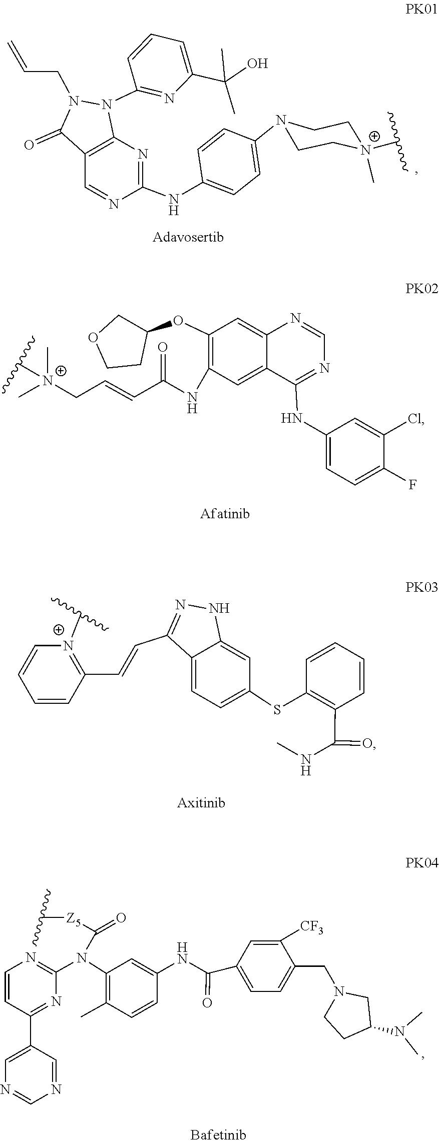

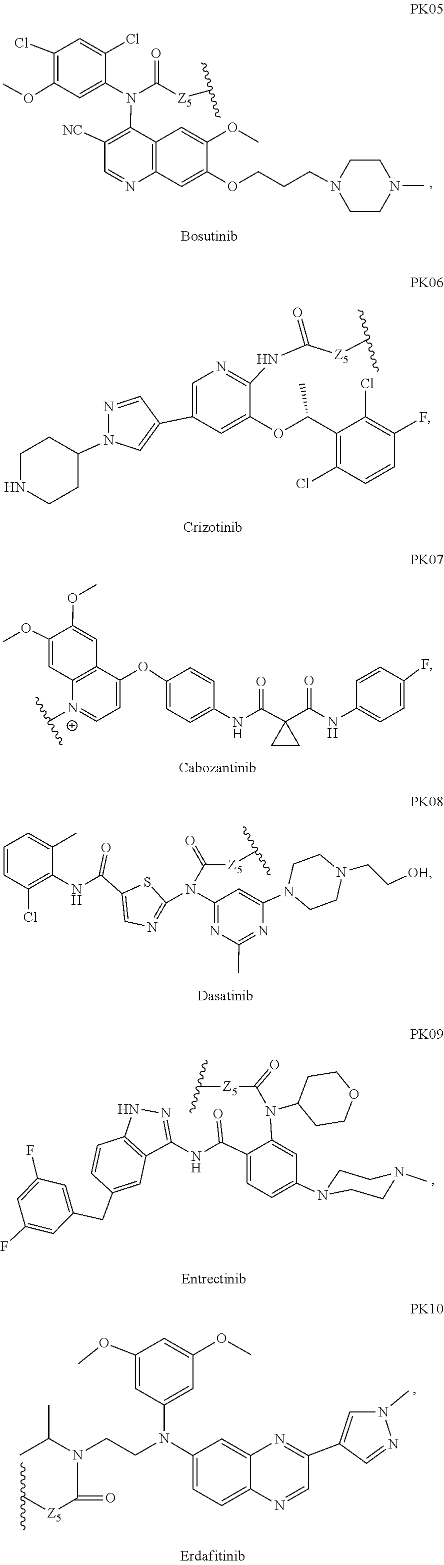

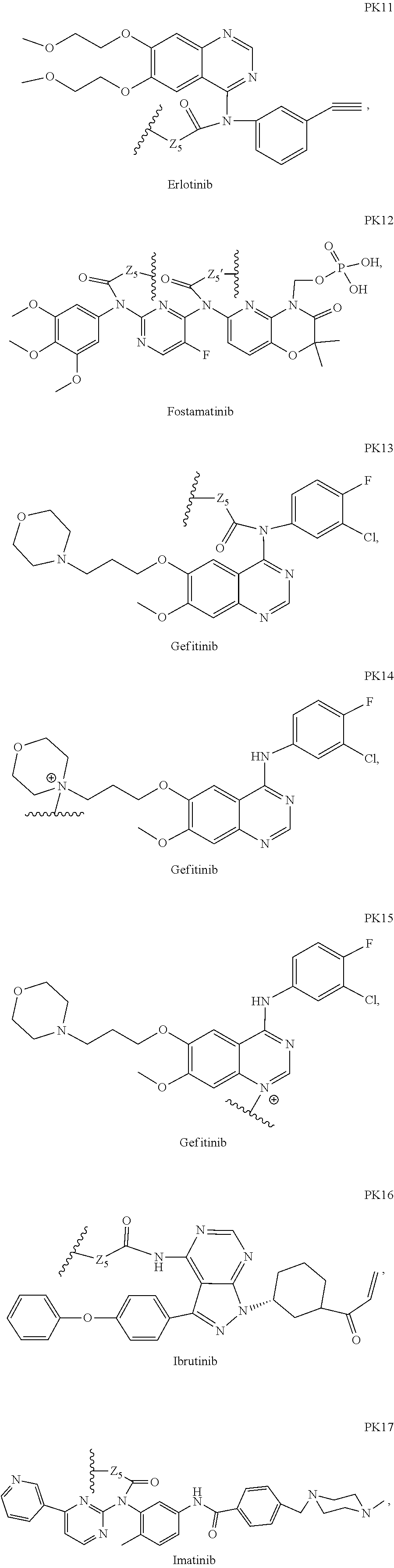

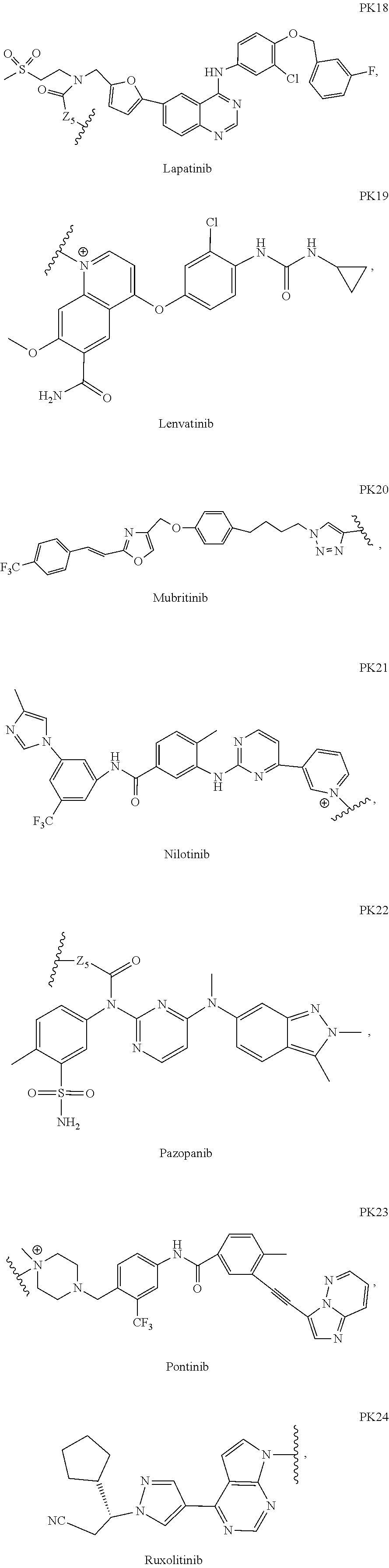

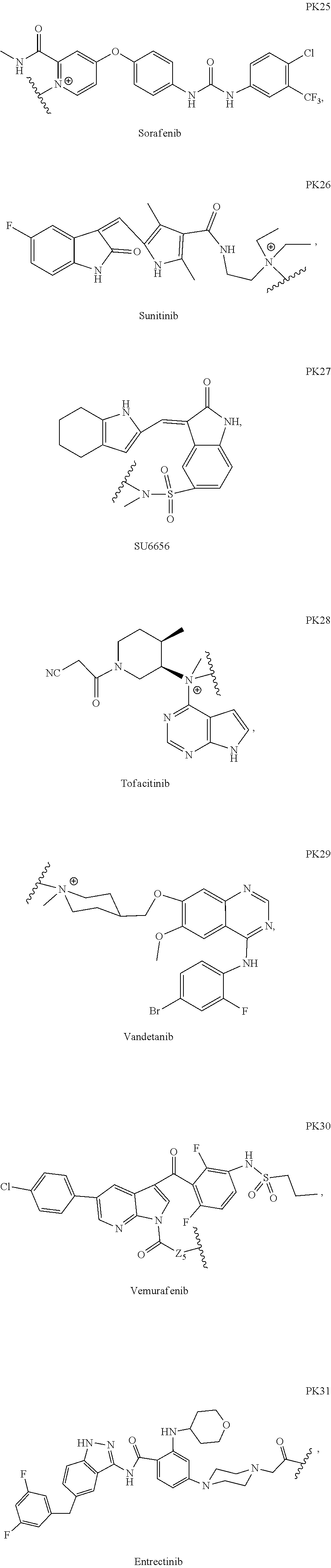

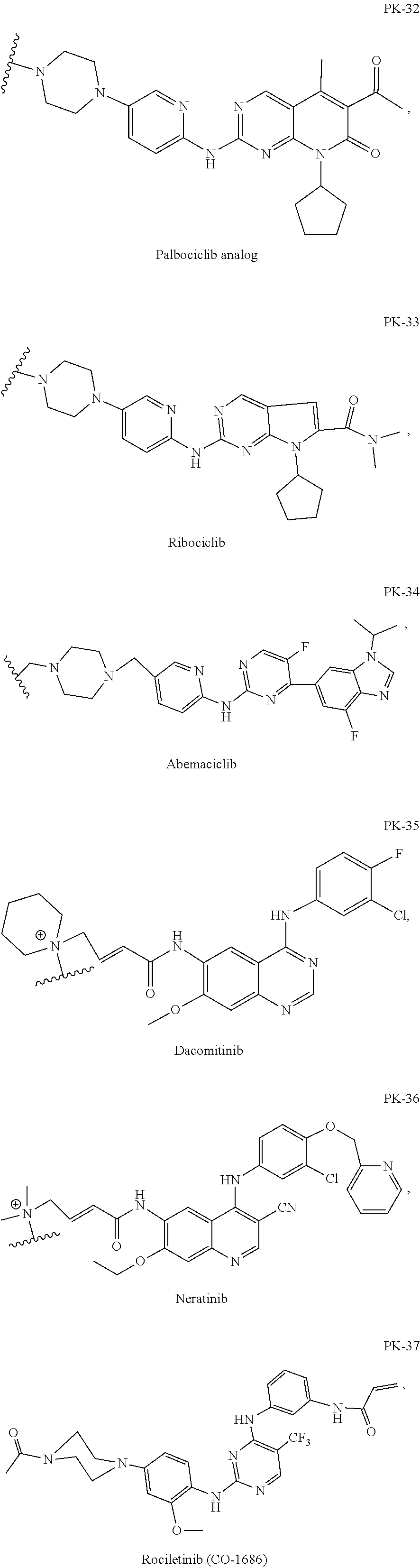

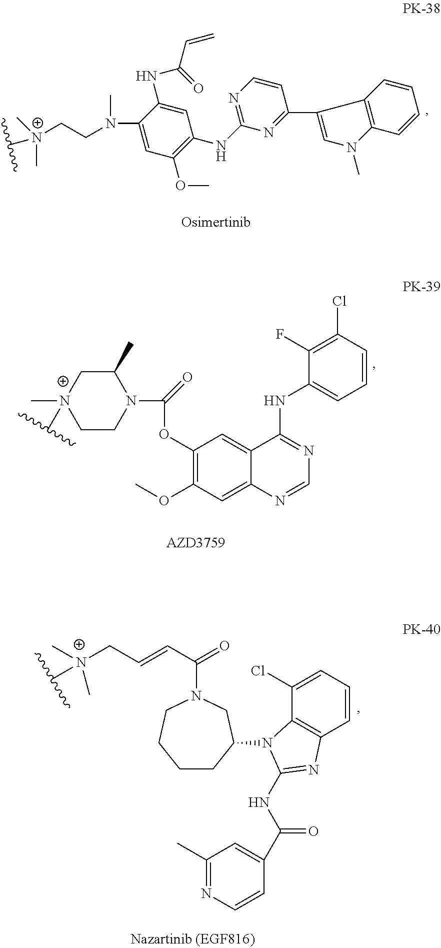

wherein Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁.

A MEK inhibitor inhibits the mitogen-activated protein kinases MEK1 and/or MEK2 which is often overactive in some cancers. MEK inhibitors are especially used for treatment of BRAF-mutated melanoma, and KRAS/BRAF mutated colorectal cancer, breast cancer, and non-small cell lung cancer (NSCLC). MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733. The preferred MEK inhibitors are selected from Trametinib (GSK1120212), Cobimetinib (XL518), Binimetinib (MEK162), selumetinib having the following formula:

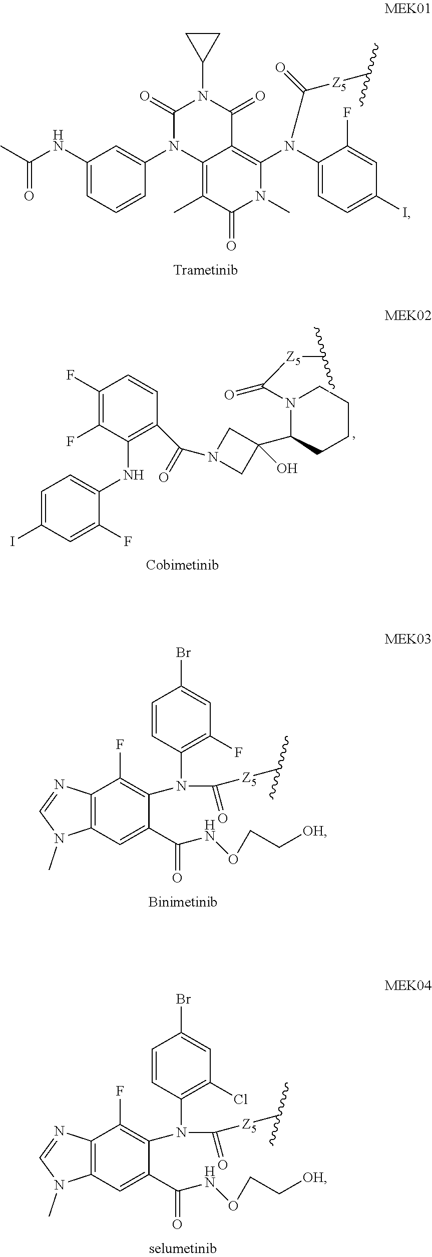

wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁;

A proteinase inhibitor that are used as a payload is preferably selected from: Carfilzomib, Clindamycin, Retapamulin, Indibulin, as shown in the following structures:

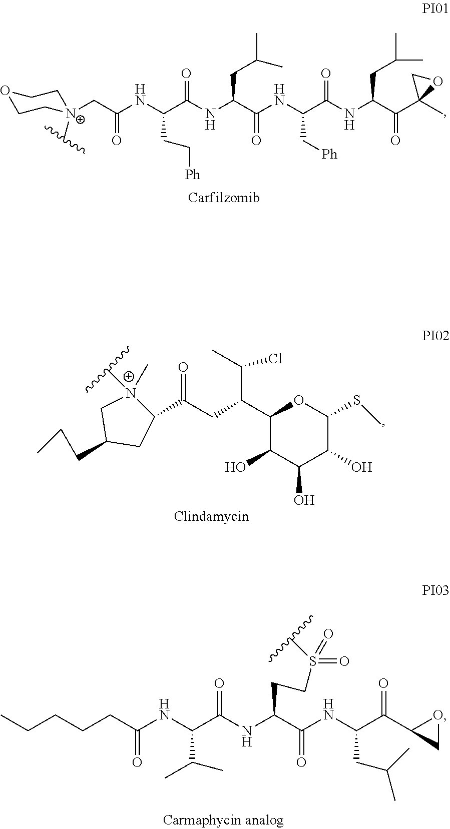

An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves; An immunotoxin herein is preferably conjugated via the process of the application through an amino acid having free amino, thiol or carboxyl acid group; and more preferably through N-terminal amino acid.

In addition, a certain cell receptor agonist, a cell stimulating molecule or intracellular signalling molecule can be as a drug D conjugated via the process of the invention.

A cell-binding ligand or receptor agonist selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinizing hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gy-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gy-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of αᵥβ₁, αvβ₃, αVβ₅, αVβ₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists;

A cell-binding molecule/ligand or a cell receptor agonist selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃, integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an av integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) which are shown in the following structures:

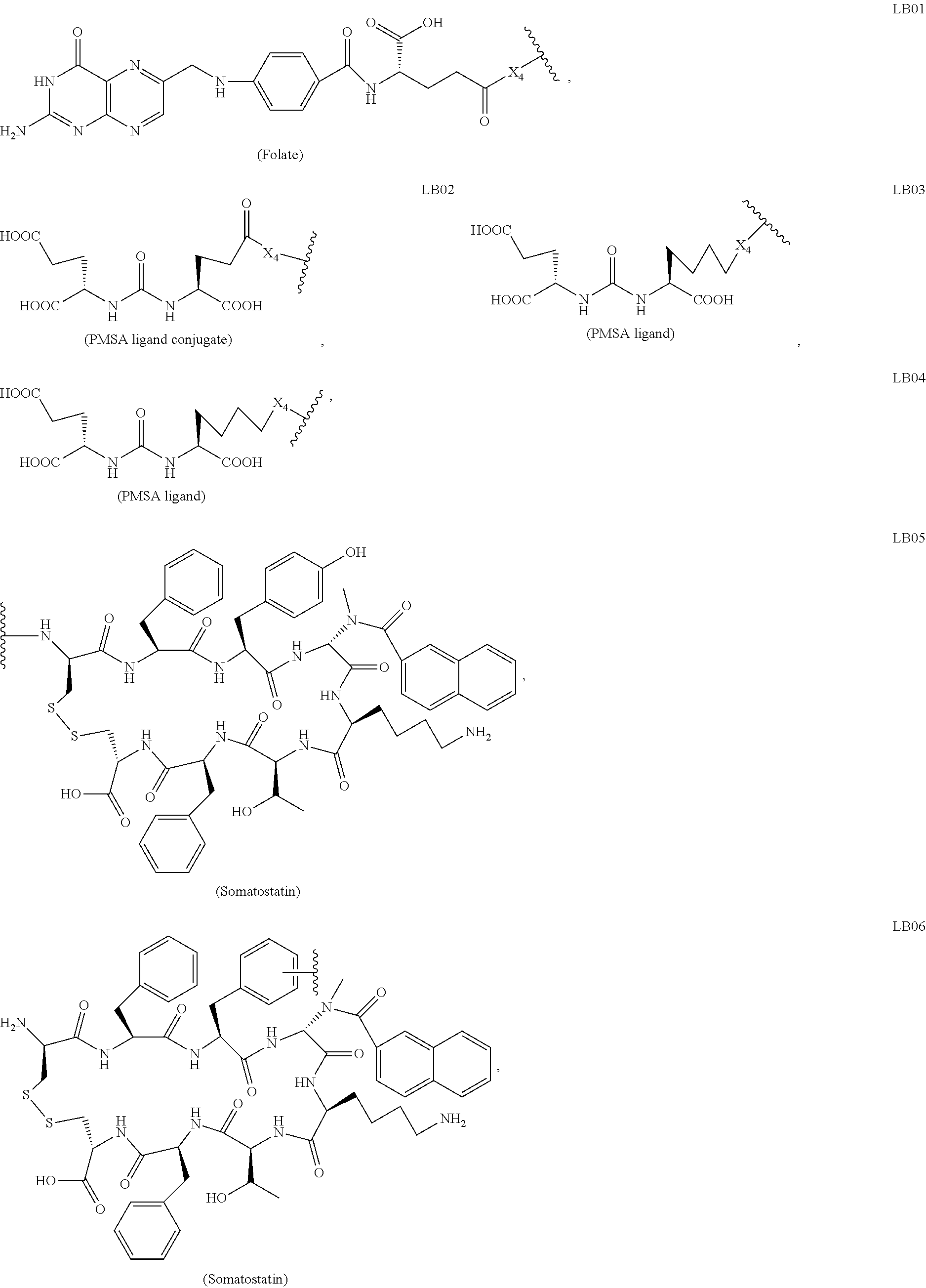

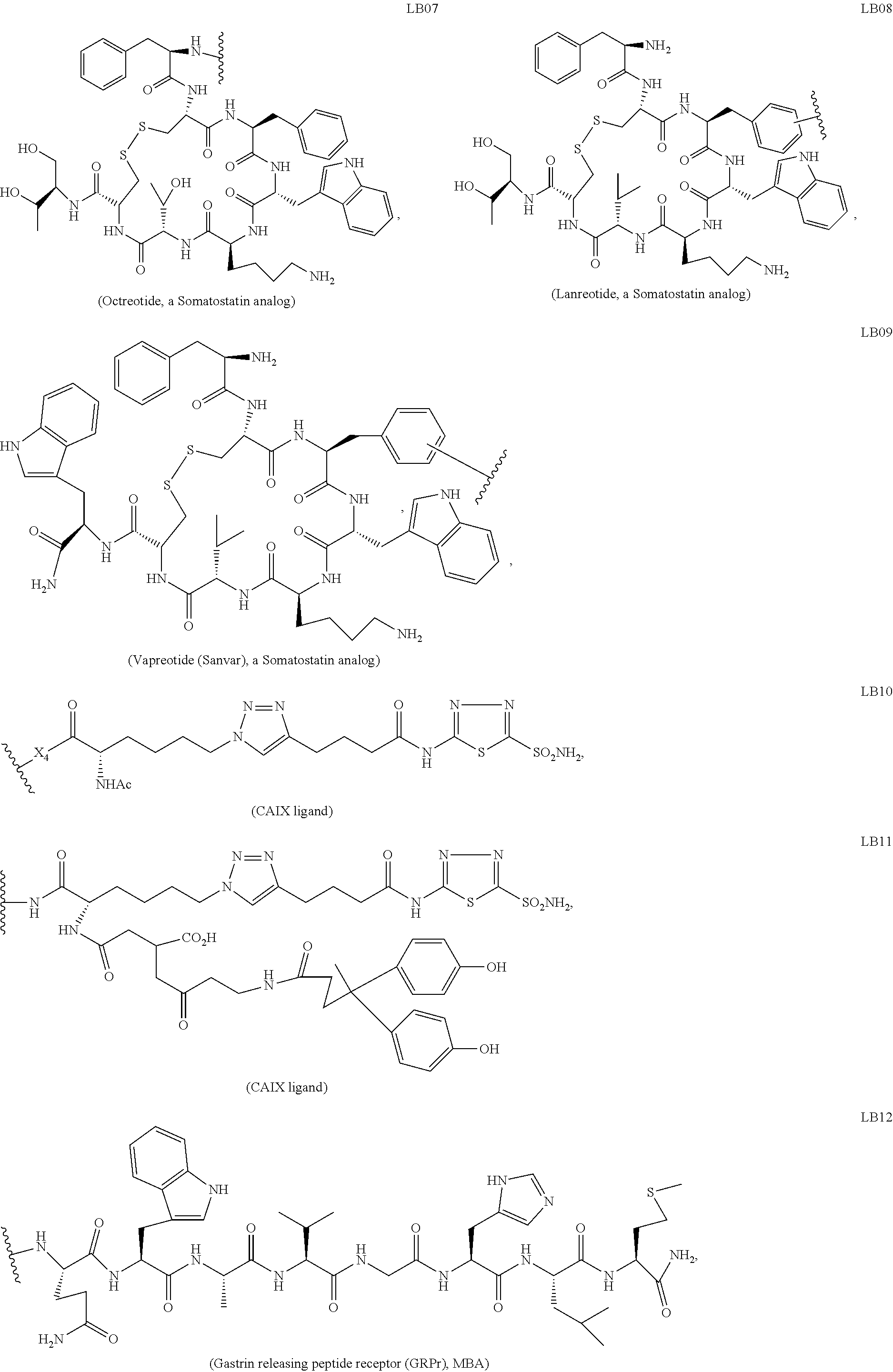

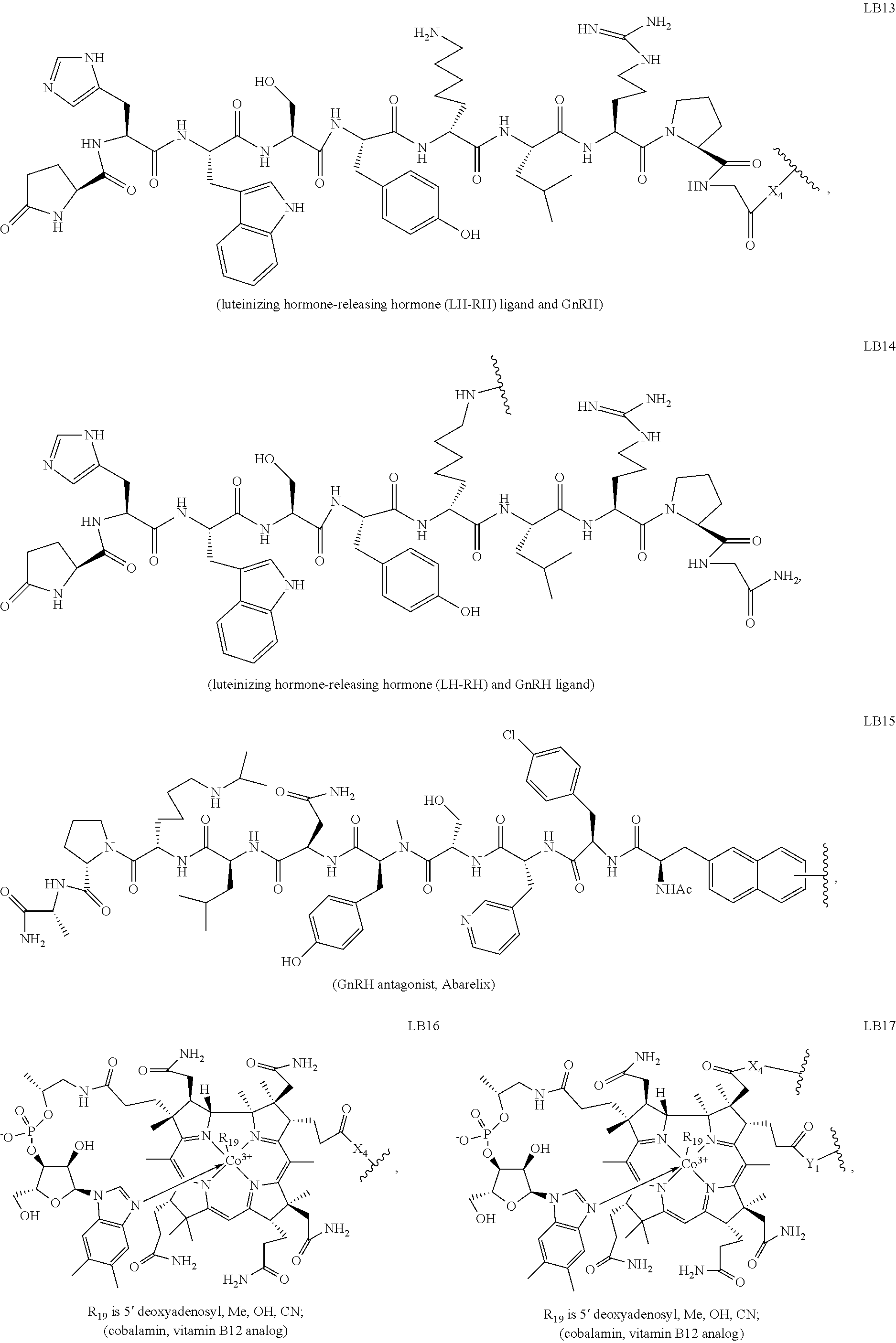

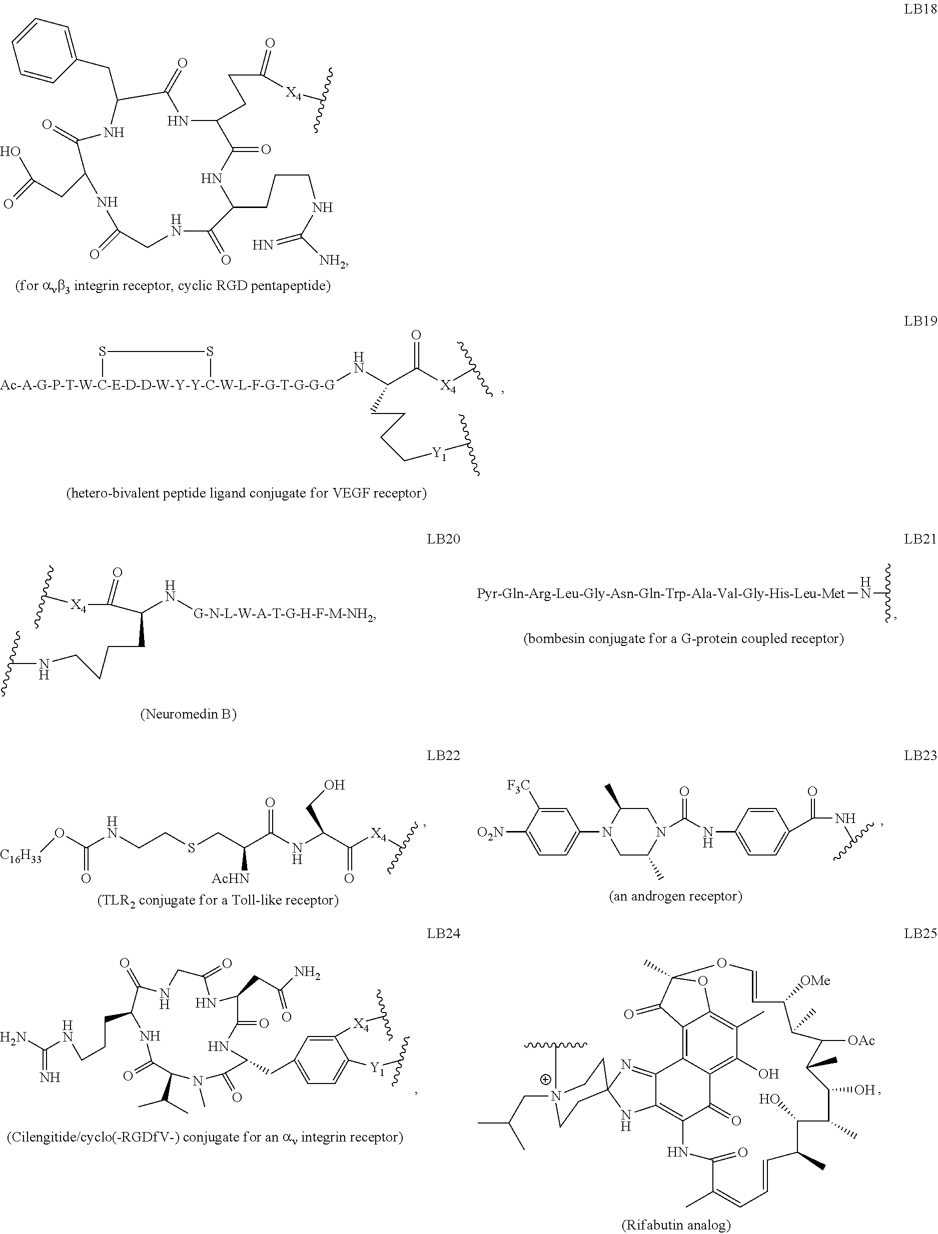

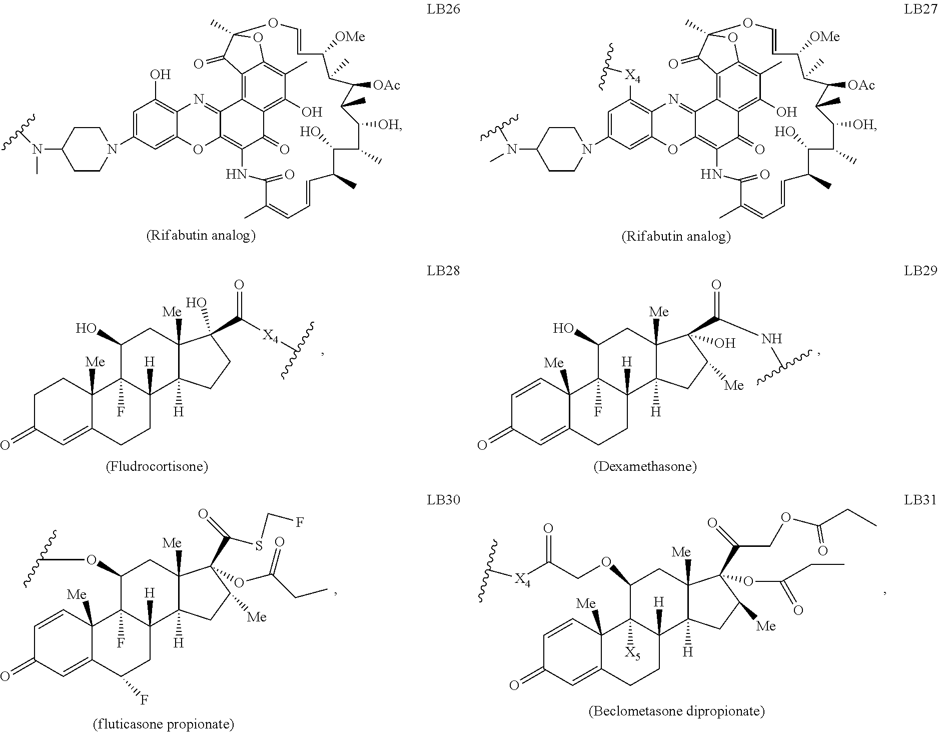

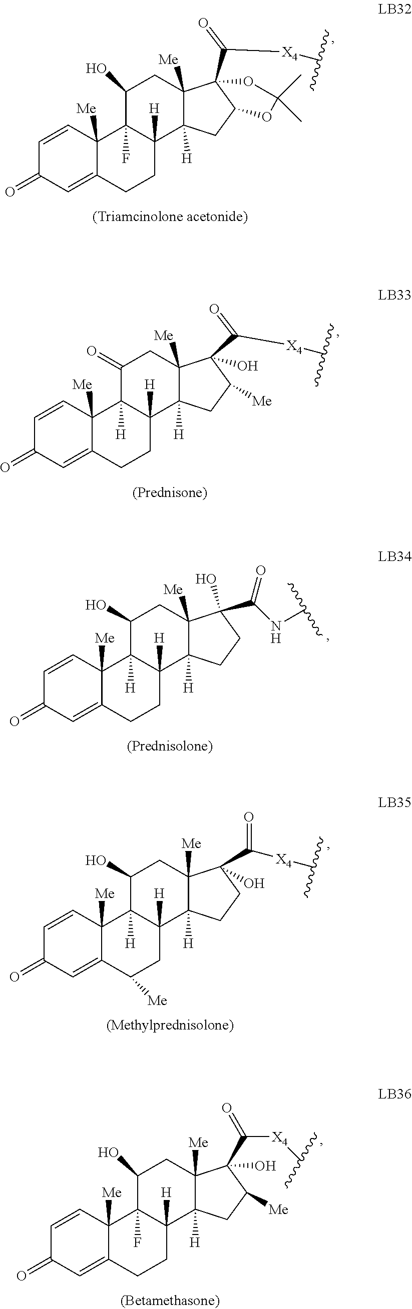

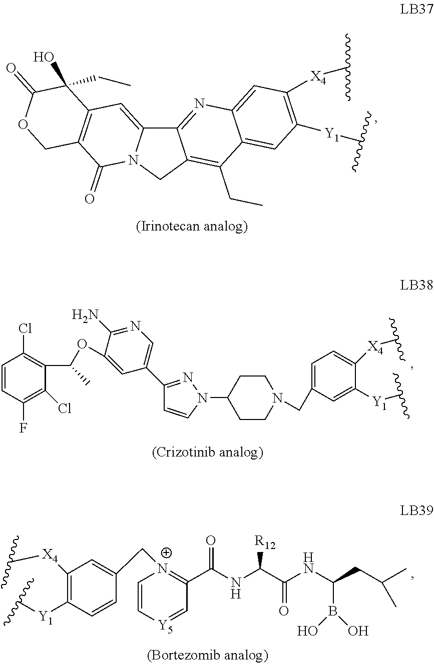

wherein Y₅, is N, CH, C(Cl), C(CH₃), or C(COOR₁); R₁₂ is H, C₁-C₆ Alkyl, C₃-C₈ Ar;

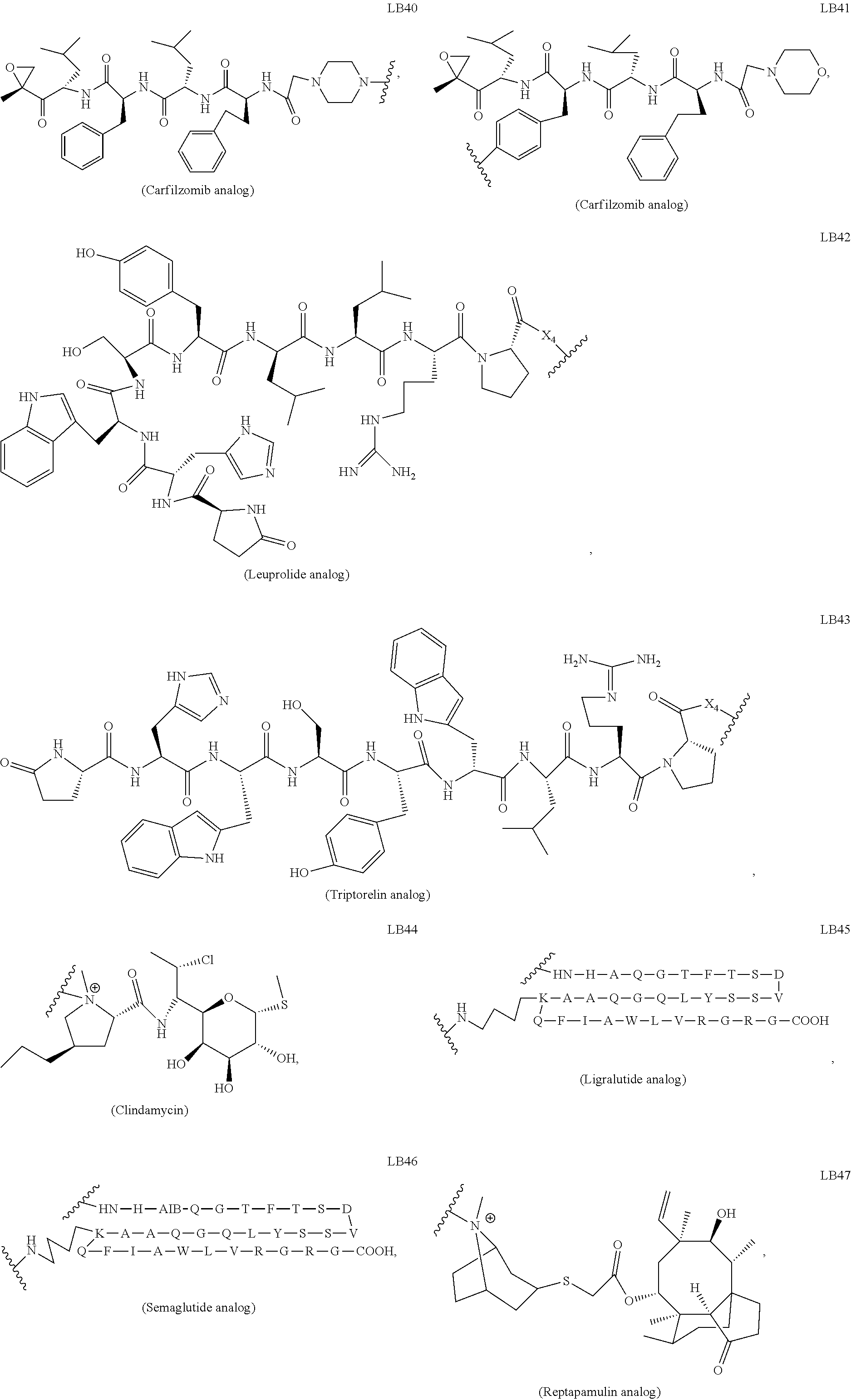

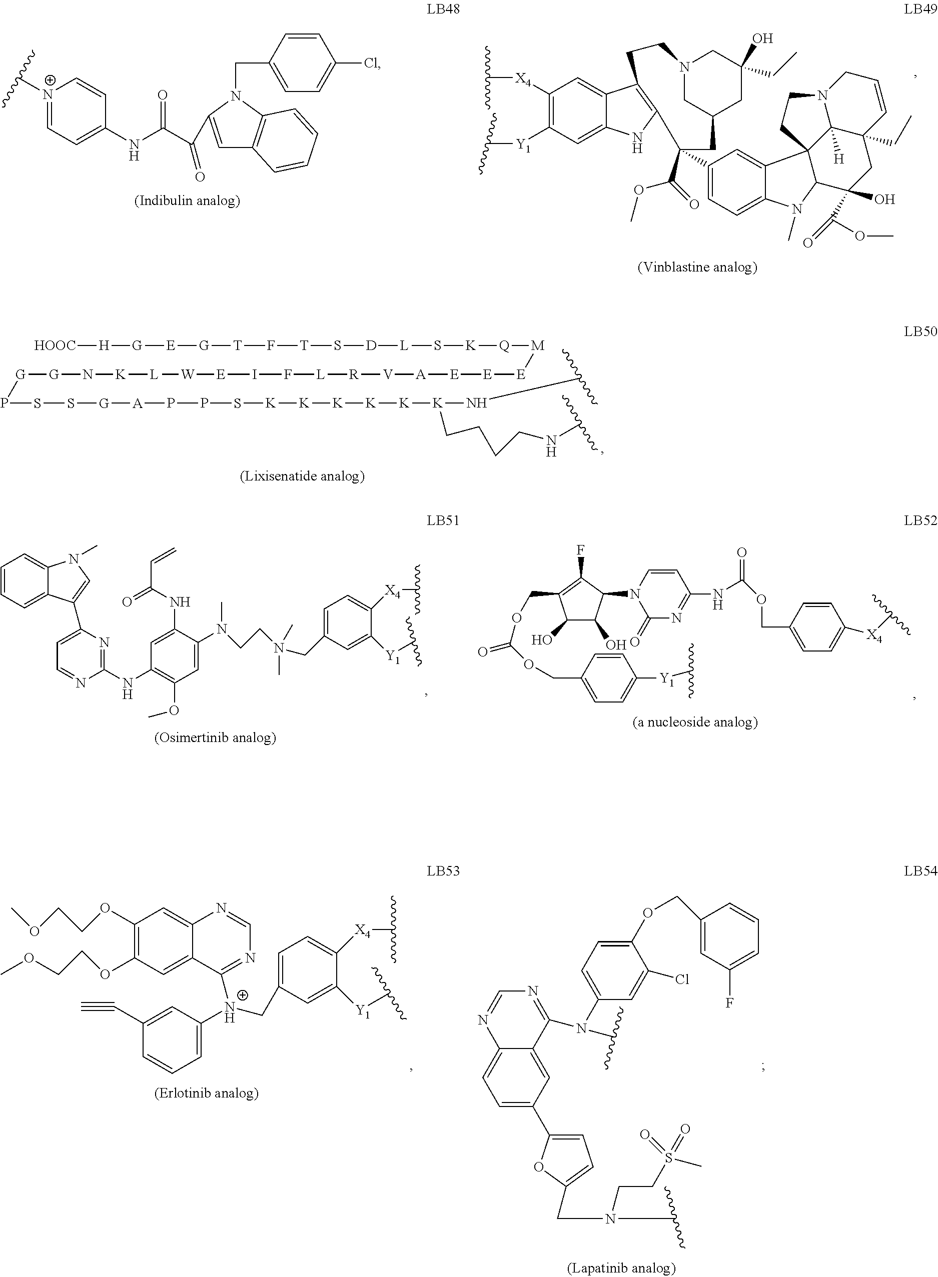

Wherein X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

In certain embodiments, one, two or more DNA, RNA, mRNA, small interfering RNA (siRNA), microRNA (miRNA), and PIWI interacting RNAs (piRNA) can be as a drug conjugated via the process of the invention:

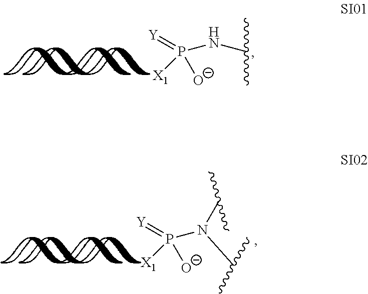

wherein “ [symbol] ” is the site to link the side chain linker of the present patent; [symbol] is single or double strands of DNA, RNA, mRNA, siRNA, miRNA, or piRNA; X₁, and Y are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

In certain embodiments, the oxidant which can be added in step (c) (after the step of the conjugation reaction) in the process of invention is preferably selected from dehydroascorbic acid (DHAA) to re-oxidize unreacted thiol groups, thus leading to restore the disulfide linkage in the antibody or antibody-like protein for having longer half life. The concentration of the oxidant in the reaction solution may be 0.01 mM-1.0 mM.

Instead of addition of DHAA in the process of invention, an excess amount of disulfide compound, such as cystine can be added in the step (c) to replace DHAA. Thus the disulfide compound can be reduced by the excess reductant, such as TCEP in step (b), to form a thiol compound, which simultaneously reacts to the excessive conjugation linker or linker/payload complex containing thiol reactive groups (e. g. maleimide), and following by removing of the generated thiol-succinimide linker/payload complex by chromatography.

In some embodiments, under conjugation process of the present invention to introduce specific thiols in the antibody or antibody-like protein (Preferably only the disulfide bonds between heavy-light chain when the antibody or antibody-like protein is IgG antibody are reduced under conjugation process of the present invention to generate the thiols), then the thiols simultaneously or sequentially in conjugation process react to formula (I), (II) or (III) independently to an antibody or antibody-like protein to form the conjugates of formula (V), (VI), or (VII) as represented below:

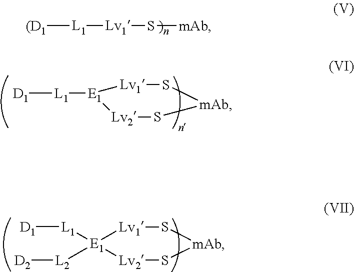

wherein n is 1-20; n′ is 1-10; preferably n is 1-8 and n′ is 1-4; more preferably n is 2-4 and n′ is 1-2; D₁, D₂, L₁, L₂, and E₁ are described the same above; S (sulfur) is generated from the reduction of disulfide bonds in the antibody-like protein (e.g. antibody) under process of the invention; mAb is an antibody-like protein; Wherein Lv₁′ and Lv^{2′} are independently the resulting groups that a thiol in mAb reacted with Lv₁ and Lv₂, whose structures described above. Wherein Lv₁′ and Lv^{2′} are independently having the following structures:

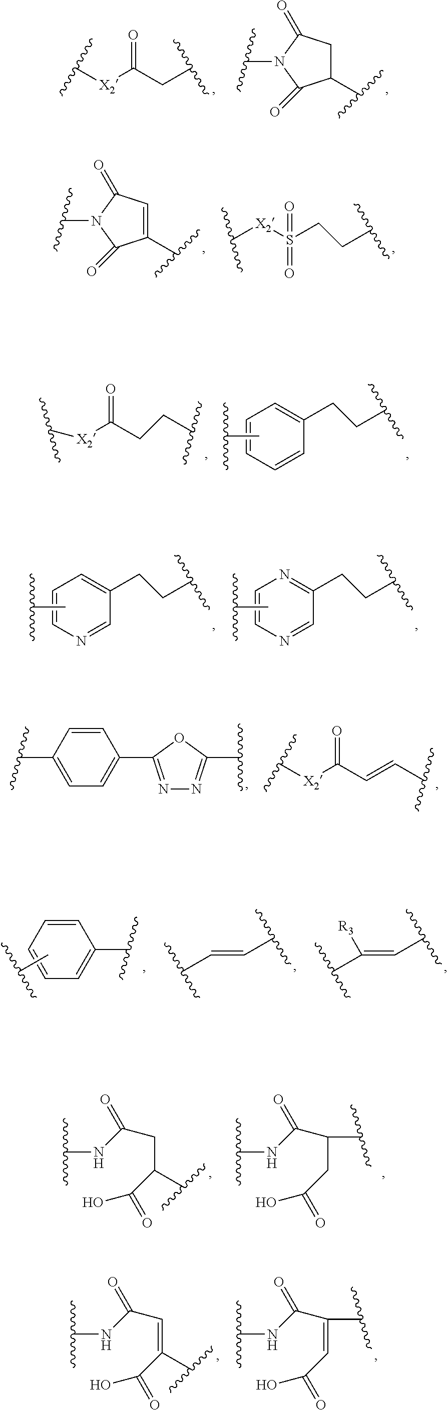

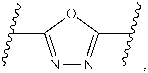 [SMILES: *c1nnc(*)o1]

wherein X^{2′} and R₃ are defined the same previously.

In the formula (VI) and formula (VII) wherein the fragments of,

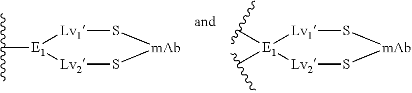

can independently be selected from:

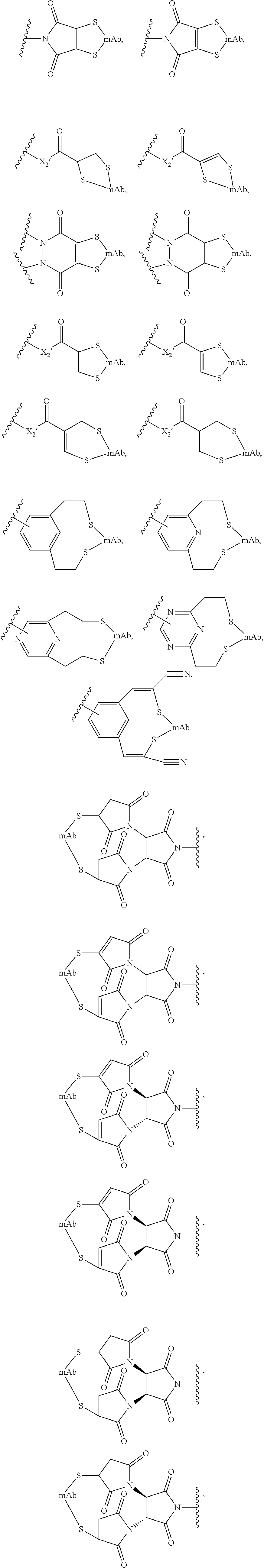

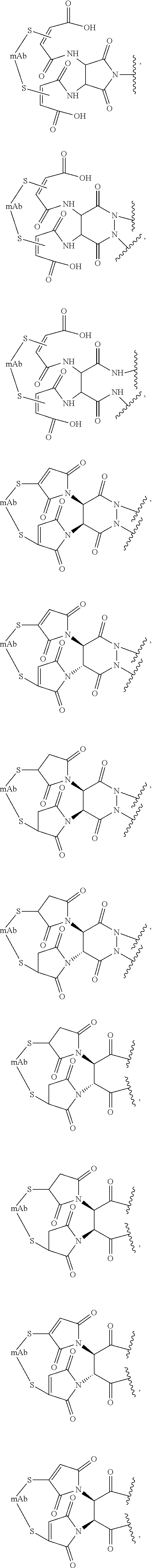

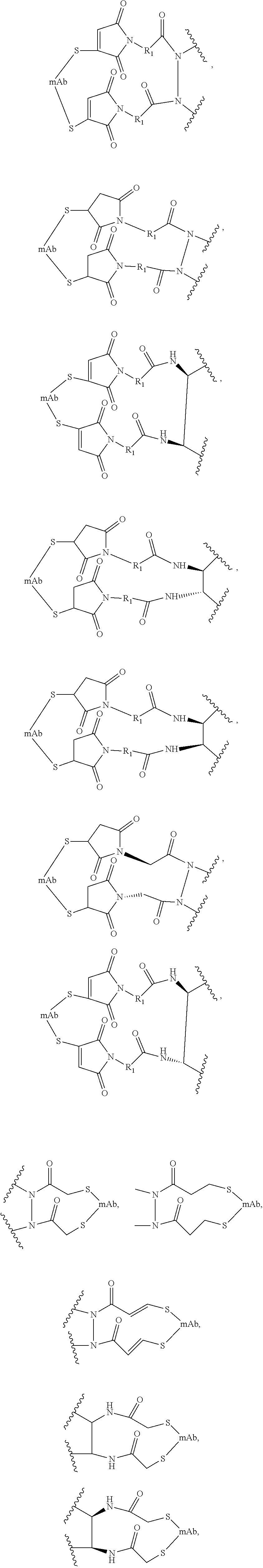

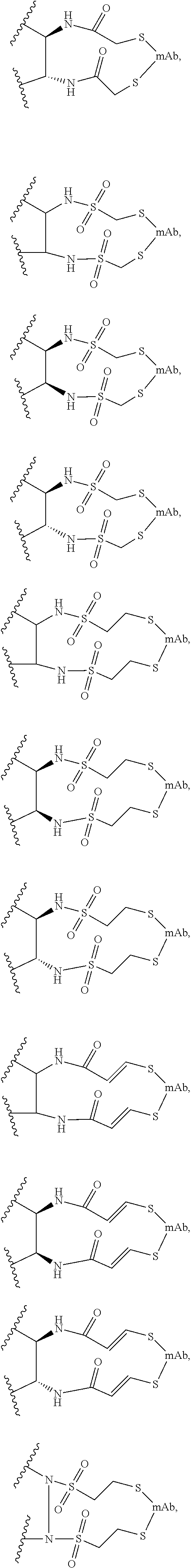

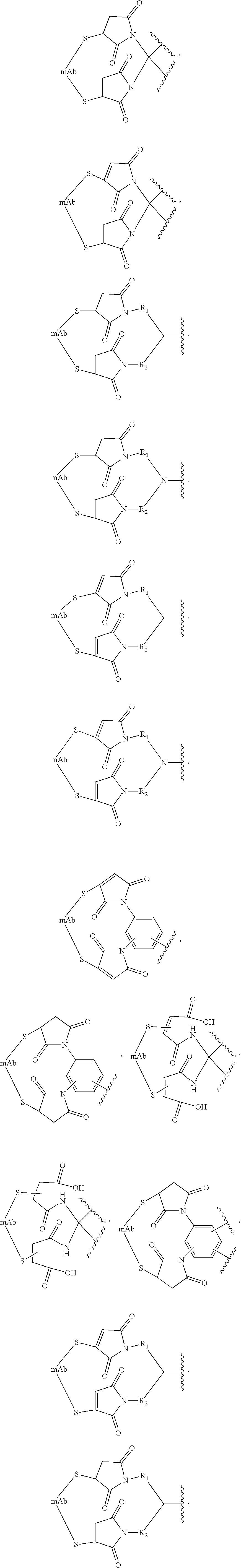

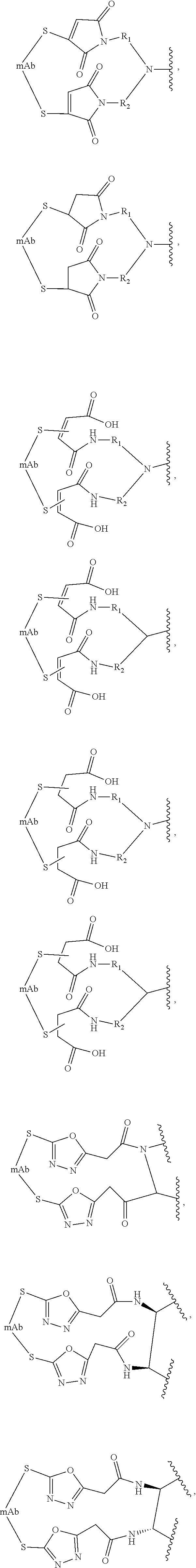

wherein R₁, R₂, X₂ are defined the same above; mAb is an antibody-like protein, preferably an antibody. Preferably, the conjugates are specifically linked to the tools between heavy-light chains of the antibody when an antibody-like protein is specifically an antibody.

In some embodiments, under process of the present patent invention, wherein a linker having formula (VIII), (IX) or (X) illustrated below can react first to the selectively reduced thiols in the antibody or antibody-like protein (e. g. typically thiols between heavy-light chain when the antibody or antibody-like protein is IgG antibody) independently, followed by condensation with a cytotoxic drug or cytotoxic drug/linker complex to form the conjugates of formula (V), (VI), or (VII) as shown above:

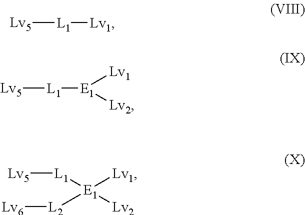

Wherein L₁, L₂, E₁, Lv₁, and Lv₂ are defined the same above for Formula (I), (II) and (III); wherein Lv₅ and Lv₆ are independently selected from

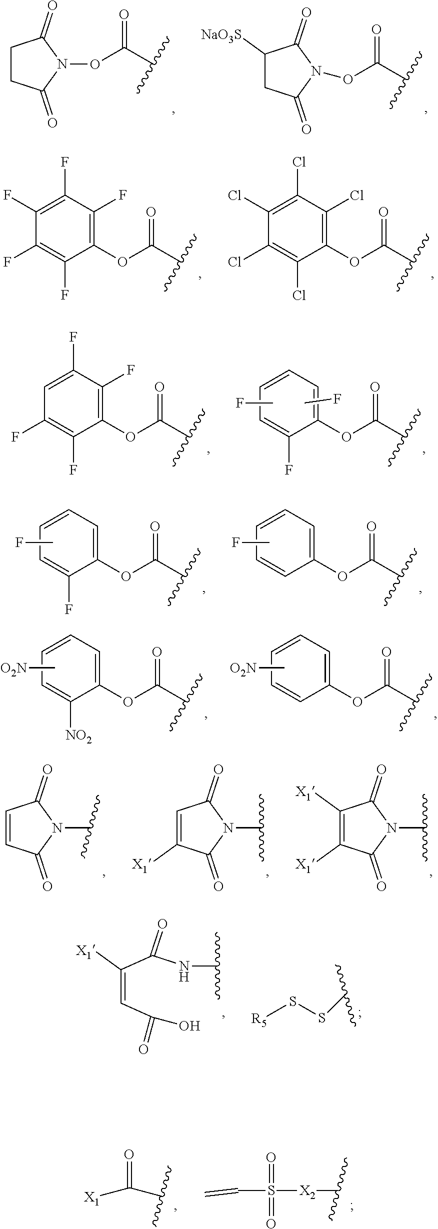

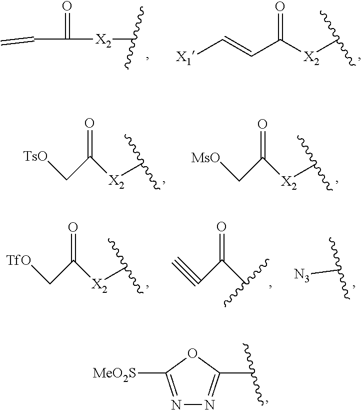

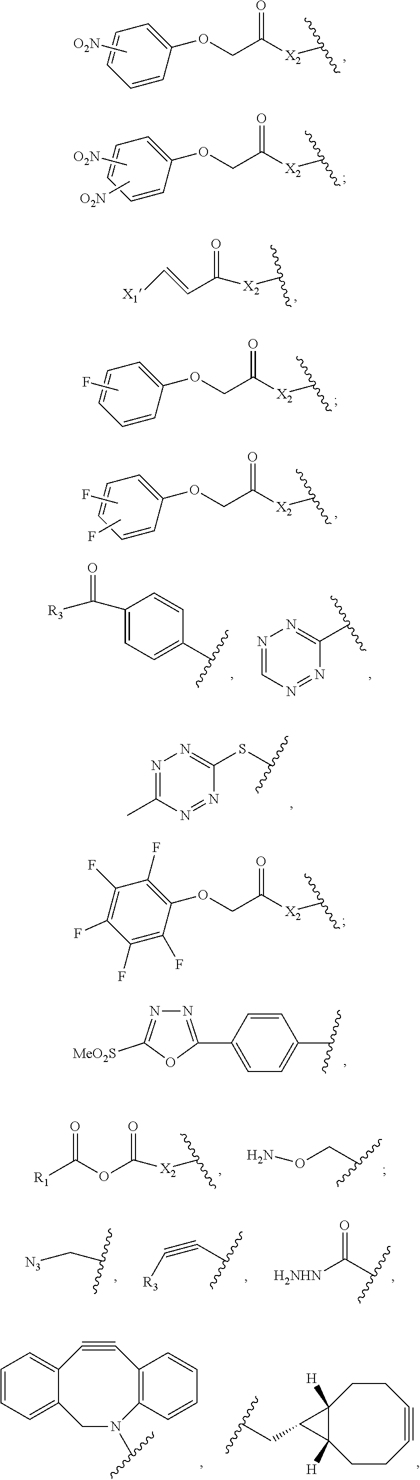

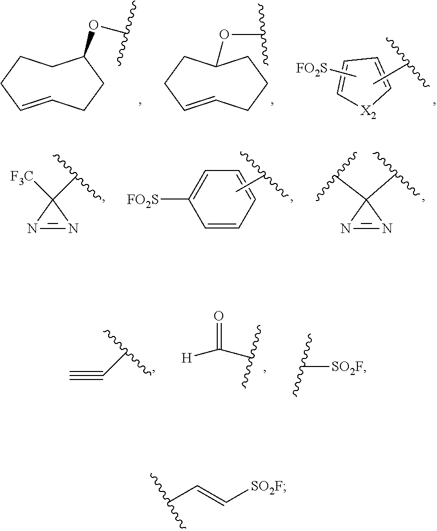

wherein X₁′ is F, Cl, Br, I, OTs (tosylate), OTf (triflate), OMs (mesylate), OC₆H₄(NO₂), OC₆H₃(NO₂)₂, OC₆F₅, OC₆HF₄, or Lv₃; X^{2′} is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions; wherein the function groups Lv₅ and/or Lv₆ can be also reacted with a thiol in a cytotoxic drug as long as the reaction are at least one fold faster or slower than the reaction between Lv₁ or Lv₂ and a thiol in an antibody-like protein, in particular, in an antibody.

In some embodiments, under process of the present patent invention introducing specific thiols in the antibody-like protein, typically generating thiols between heavy-light chain when the antibody or antibody-like protein is IgG antibody, then the thiols simultaneously or sequentially in the conjugation process react to the linker of formula (VIII), (IX) or (X) illustrated above to form the antibody-like protein/linker complex molecule of formula (XI), (XII) or (XIII) below, following by reaction with a a cytotoxic drug D₁ or D₂ independently to form the conjugate of formula (V), (VI), or (VII).

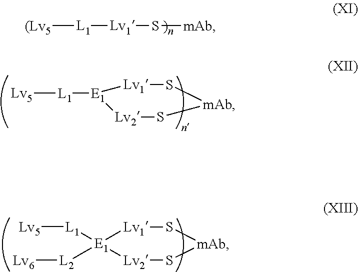

wherein Lv₅, Lv₆, L₁, L₂, E₁, Lv₁′ Lv^{2′}, mAb, n and n′ are described the same above.

In some embodiments, under process of the present patent invention, wherein the linker of formula (VIII), (IX) or (X) illustrated above can react first with a cytotoxic drug to form the cytotoxic drug/linker complex molecule of formula (I), (II) or (III), follow by reaction with the reduced thiols in the antibody or antibody-like protein independently to form the conjugate of formula (V), (VI), or (VII) under process of this invention. The first step condensation reaction of the formula (VIII), (IX) or (X) to a cytotoxic drug can be in a separated pot, and the resulted cytotoxic drug/linker complex molecules of formula (I), (II) or (III) can be optionally purified by a chromatography, extraction or precipitation before for conjugation to the reduced thiols in the antibody-like protein. The first step of specific reduction of disulfide bonds in an antibody-like protein and conjugation reaction with formula (I), (II) or (III) are preferred in the same pot without separation of intermediates.

To distinguish the reactions between Lv₅ and/or Lv₆ to a cytotoxic drug, and Lv₁ and/or Lv₂ to a thiol in an antibody-like protein, each step of the reactions for the linker of formula (VIII), (IX) or (X) can be conducted at different conditions in the same or different reaction pots. For instance, a drug containing an amino group can undergo condensation with a carboxylic acid group in the linker in the present of a condensation regent, e. g. EDC, TBTU or BroP, to give a modified drug/linker complex of Formula (I), (II) or III) bearing amide bonds. This condensation reaction can be performed at physiological buffer solution wherein the carboxylic acid group at one terminal of the linker of formula (VIII), (IX) or (X) is activated to be N-hydoxylsuccinimidyl (NHS), pentfluorophenyl, dinitrophenyl ester, or carboxylic acid chloride group, etc, which can react to a drug bearing an amino group to provide drug/linker complex of Formula (I), (II) or III), then subsequently or simultaneously undergo the conjugation to thiols of an antibody-like protein according to the process of the present application to form the conjugate of formula (V), (VI), or (VII). In another practice, the linker of formula (VIII), (IX) or (X) bearing both a thiol reactive group (e. g. maleimido, vinylsulfonyl, haloacetyl, acrylic, substituted propiolic) at one terminal and a drug reactive group (e. g. hydoxylsuccinimidyl (NHS), pentfluorophenyl, dinitrophenyl ester, amino, alkyloxylamino or clickable chemistry group (e. g. azide, alkyne, dibenzocyclooctyne, BCN ((1R, 8S, 9s)-bicyclo[6.1.0]non-4-yn-9-ylmethanol)) at the other terminal can undergo undergo the conjugation to thiols of an antibody-like protein (preferably an antibody) according to the process of the present application in a buffer solution at pH 4.5-7.5, 2° C.-40° C. (preferably 2° C.-8° C.) with or without addition of 0˜30% of water mixable (miscible) organic solvents to form the antibody-like protein-linker conjugate of formula (XI), (XII) or (XIII) independently. Then a drug bearing a reactive group matched to the reactive group in the antibody-like protein-linker conjugate of formula (XI), (XII) or (XIII) accordingly can be subsequently or simultaneously added to the reaction solution to provide the conjugate of formula (V), (VI), or (VII). In the second step reaction, the antibody-like protein-linker conjugate of formula (XI), (XII) or (XIII) can be optionally purified before proceeding the condensation with a drug, and the condensation condition of the second step can be adjusted, e. g. the pH is adjusted to 6.5-8.0, and/or temperature is adjusted to 20-45° C.

In some embodiments, during the process of the conjugation of this invention, prior to conjugating with a drug, the antibody-like protein can be modified through attachment of a heterobifunctional cross linker of formula (XI), (XII) or (XIII), such as with linkers of Amine-to-Sulfhydryl (succinimidyl (NHS) ester/maleimide, NHS ester/pyridyldithiol, NHS esters/haloacetyl), diazirine (SDA)-to-Sulfhydryl, Azide-to-Sulfhydryl, Alkyne-to-Sulfhydryl, Sulfhydryl-to-Carbohydrate (Maleimide/Hydrazide, Pyridyldithiol/Hydrazide, haloacetyl/Hydrazide), Hydroxyl-to-Sulfhydryl (Isocyanate/Maleimide), Sulfhydryl-to-DNA (Maleimide/Psoralen, Pyridyldithiol/Psoralen, haloacetyl/Psoralen), Sulfhydryl-to-Carboxyl (Carbodiimide).

The reactive group of a drug/cytotoxic agent that reacting to a modified an antibody-linker conjugate of formula (XI), (XII) or (XIII) to give the final conjugate can be in different ways accordingly. For example, the conjugate linked via disulfide bonds is achieved via the first step, a linker of formula (VIII), (IX) or (X) is conjugated to the antibody-like protein at 2° C.-8° C., pH 4.5-6.0, according to the present invention of reduction and conjugation of an antibody-like protein, following by a disulfide exchange between a drug containing a free thiol group and the disulfide bond ((e. g. pyridyldithio moiety) in the linker attached to the modified antibody-like protein at pH 6.5-8.0, at 20° C.-40° C. Before the addition of the drug containing a free thiol for conjugation, the excess reduction agent (e. g. TCEP, or tri(3-hydroxylpropyl)phosphine) is preferably removed from the reaction pot. Synthesis of the conjugates linked via thioether is achieved by first reaction of a linker containing both thiol reactive terminals of maleimido or haloacetyl or ethylsulfonyl or substituted propiolic group to the thiols in an antibody which are reduced by the process of the present patent application at 2° C.-8° C., pH 4.5-6.0 to give the antibody-linker conjugate of formula (XI), (XII) or (XIII), following by reaction of a drug containing a thiol at pH 6.5-8.0, at 20° C.-40° C. to to provide the conjugate of formula (V), (VI), or (VII). If the same pH and/or temperature conditions are chosen for the two step reactions for thioether linked conjugates, the over four times equivalents of the linker containing dual terminal thiol reactive are used for the conjugation. It should be noted that the preferred methods of synthesis of the disulfide or thiol-ether linked conjugates are through the first chemical synthesis the drug-linker complex having disulfide or thiol-ether bonds of the formula (I), (II) or (III); following by reaction with the thiols in the protein (antibody) according the process of the invention. Synthesis of conjugates bearing an acid labile hydrazone linkage can be achieved by reaction of a carbonyl group with the hydrazide moiety in the linker, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997). Synthesis of conjugates bearing triazole linkage can be achieved by reaction of a 1-yne group of the drug with the azido moiety in the linker, through the click chemistry (Huisgen cycloaddition) (Lutz, J-F. et al, 2008, Adv. Drug Del. Rev. 60, 958-70; Sletten, E. M. et al 2011, AccChem. Research 44, 666-76). Synthesis of the conjugates linked via oxime is achieved by reaction of a modified antibody-like protein containing a ketone or aldehyde and a drug containing oxyamine group. A drug bearing a hydroxyl group or a thiol group can be reacted with a modified linker of Formula (XI), (XII), or (XIII), bearing a halogen, particularly the alpha halide of carboxylates, in the presence of a mild base, e.g. pH 8.0˜9.5, to give a modified drug/linker complex bearing an ether or thiol ether linkage of Formula (I), (II), or (III). A drug containing a hydroxyl group can be condensed with a linker of Formula (XI), (XII), or (XIII) bearing a carboxyl group, in the presence of a dehydrating agent, such as EDC or DCC, to give ester linkage, then the subject drug/linker complex undergoes the conjugation with an antibody-like protein under the process of the present invention. A drug containing an amino group can condensate with a carboxyl ester of NHS, imidazole, nitrophenoxyl; N-hydroxysuccinimide (NHS); methylsufonylphenoxyl; dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxyben-zotriazole; tosylate; mesylate; 2-ethyl-5-phenylisoxazolium-3′-sulfonate on the antibody-like protein-linker of Formula (VIII), or (XI) to give a conjugate via amide bond linkage of Formula (V), (VI), or (VII).

In further embodiments, under process of the present patent invention, the resulted conjugates of formula (V), (VI), or (VII) are over 75% linked to the cysteine sites between heavy-light chains of an antibody, and are less than 15% linked to the cysteine sites between heavy-heavy chains (hinge region) of an antibody. Typically, for formula (V) or (VII), when drug/antibody ratio (DAR) is set to be 4, the distributions in percentage of the numbers of drugs in the antibody are: D0<1%, D2<10%, D4>75%, D6<10%, D8<10%; for formula (VI), when drug/antibody ratio (DAR) is set to be 4, the distributions in percentage of the numbers of drugs in the antibody are: D0<1%, D1<10%, D2>75%, D3<10%, D4<10%.

The resulted conjugate may be purified by standard biochemical means, such as gel filtration on a Sephadex G25 or Sephacryl S300 column, adsorption chromatography, ion (cation or anion) exchange chromatography or by dialysis (ultrafiltration or hyperfiltration (UF) and diafiltration (DF)). In some cases, a small size molecule of antibody-like protein (e.g. <10 KD) conjugated with a small molecular drugs can be purified by chromatography such as by HPLC, medium pressure column chromatography or ion exchange chromatography.

In general, the conjugate of Formula (V), (VI), or (VII) is preferably generated from a drug/linker complex of Formula (I), (XII), or (XIII), as in a one pot reaction. When a thiol reduced from an antibody-like protein reacts a thiol reactive group in the terminal of drug/linker complex of Formula (I), (XII), or (XIII), the Ellman reagent can be optionally used to monitor the efficient reduction of the disulfide bonds and conjugation of the tiols through measurement of the numbers of the free thiols during the reactions. A UV spectrometry at wavelength of range 190-390 nm, preferably at 240-380 nm, more preferably at 240-330 nm is preferred to be used in assisting the reaction (via monitoring the conjugation). The conjugation reaction can be thus measured or conducted in a quartz cell or Pyrex flask in temperature control environment. The drug/protein (antibody) ratios (DAR) of the conjugates can also be measured by UV at wavelength of range 240-380 nm via calculation of the concentrations of the drug and the protein, by Hydrophobic Interaction Chromatography (HIC-HPLC) via measurement of the integration areas of each drug/protein fragment, by Capillary electrophoresis (CE), and/or by LC-MS or LC-MS/MS or CE-MS (the combination of liquid chromatography (LC) or CE with mass spectrometry (MS) via measurement of both the integration areas of LC or CE and Peak intensity of MS for each drug/protein fragment). It is also noted in the conjugation process of the present invention, when a drug or a drug/linker complex is not well soluble in a water based buffer solution, up to 30% of water mixable (miscible) organic solvents, such as DMA, DMF, ethanol, methanol, acetone, acetonitrile, THF, isopropanol, dioxane, propylene glycol, or ethylene diol can be added as the co-solvent in water based buffer solution.

The aqueous solutions for the modification of the antibody-like protein are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, sucrose and salts, for examples, NaCl and KCl. Other biological buffers that are used for the conjugation process are listed in the definition section. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding antibody-like protein agent can be performed in a routine way, using for example gel filtration chromatography, or adsorptive chromatography.

When disulfide exchange reaction is used for modification of an antibody-like protein, the extent of the modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by UPLC-QTOF mass spectrometry, or Capillary electrophoresis-mass spectrometry(CE-MS). The linker compounds have diverse functional groups that can react with drugs, preferably cytotoxic agents that possess a suitable substituent. For examples, the modified antibody-like protein bearing an amino or hydroxyl substituent can react with drugs bearing an N-hydroxysuccinimide (NHS) ester, the modified antibody-like protein bearing a thiol substituent can react with drugs bearing a maleimido or haloacetyl group. Additionally, the modified antibody-like protein bearing a carbonyl (ketone or aldehyde) substituent can react with drugs bearing a hydrazide or an alkoxyamine. One skilled in the art can readily determine which linker to use based on the known reactivity of the available functional group on the linkers.

Examples of preferred conjugates of formula (V), (VI), and (VII) which can be constructed under the process of the invention to achieve over 80% of the total drugs linked to the cysteines between heavy-light chains of the antibody are illustrated below:

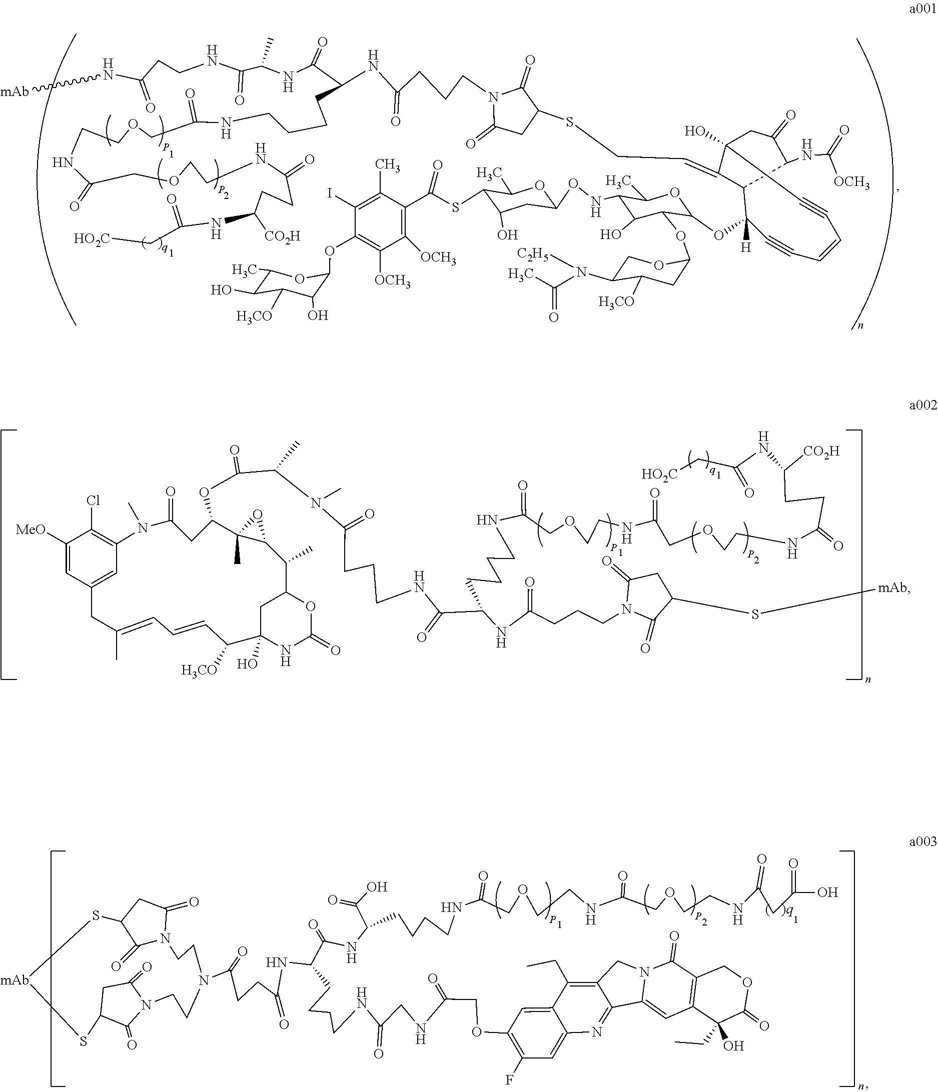

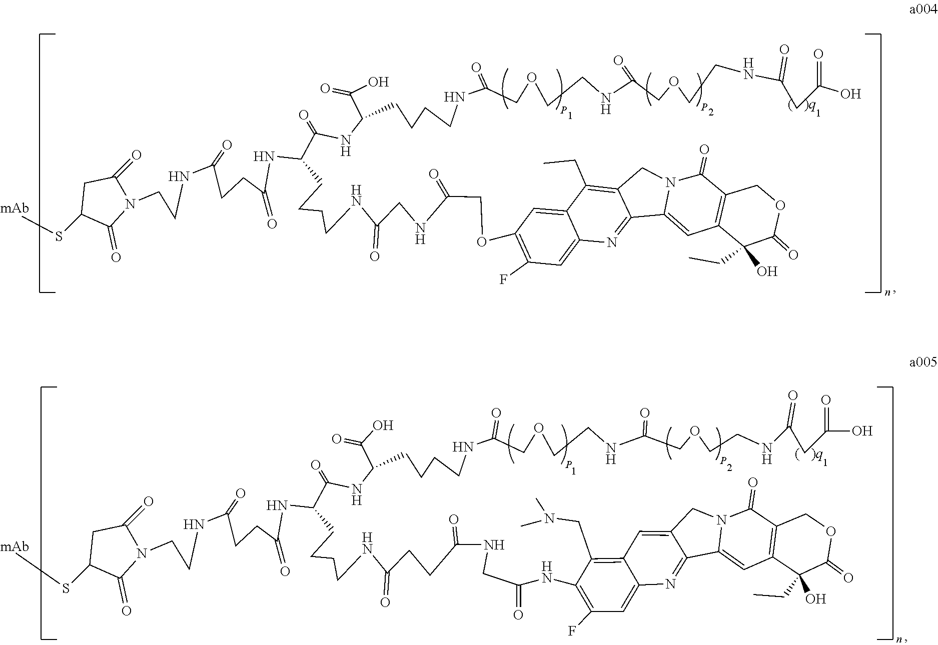

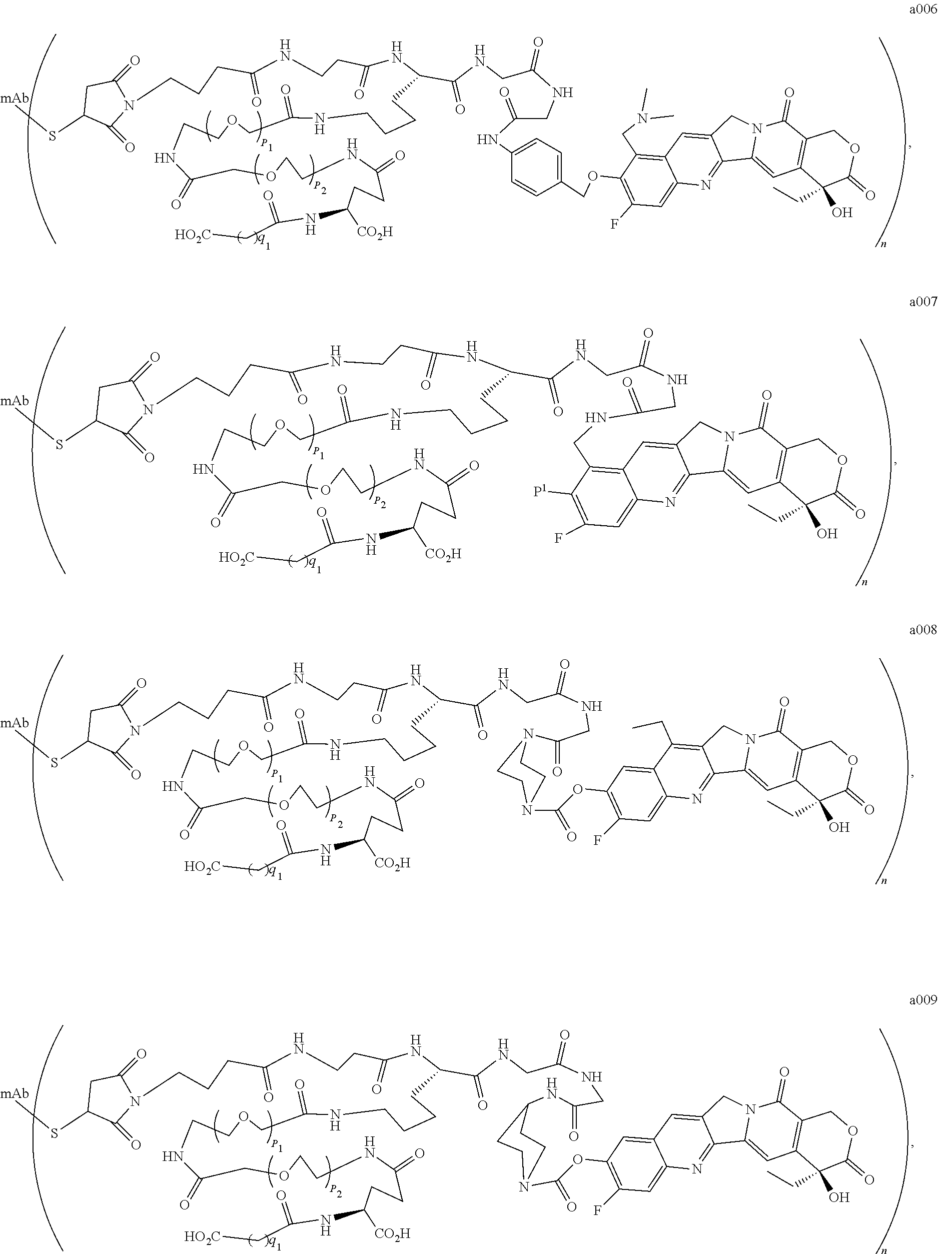

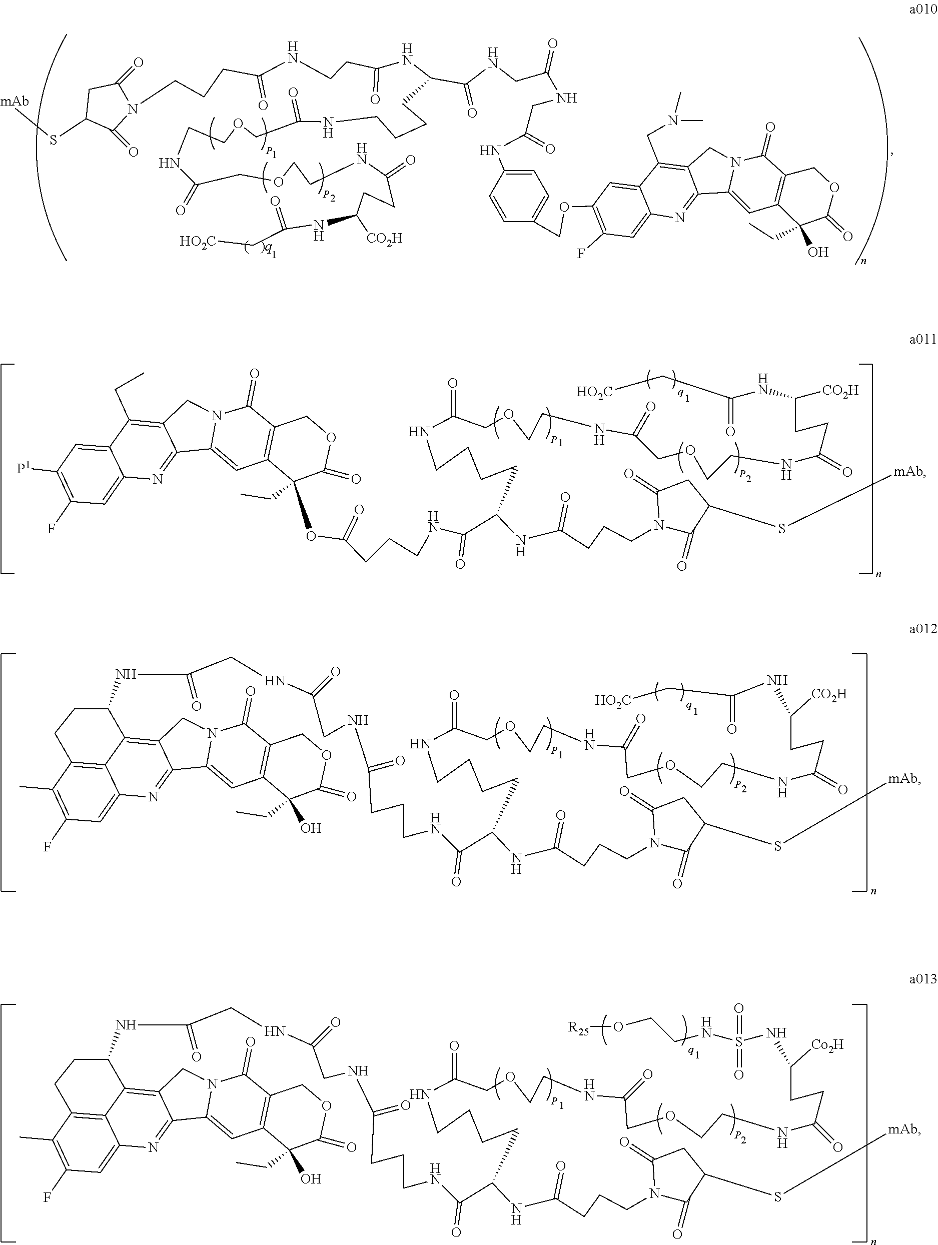

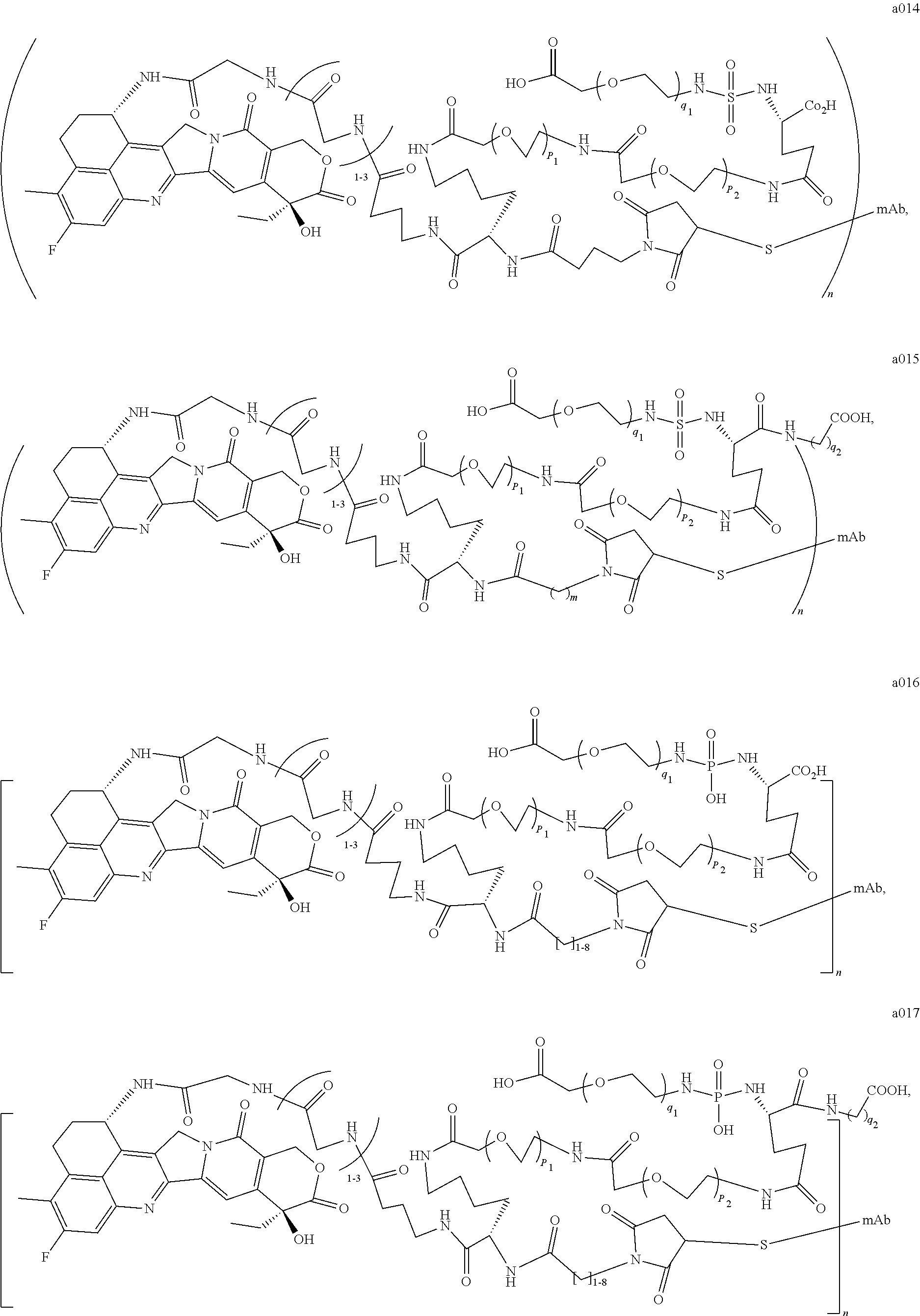

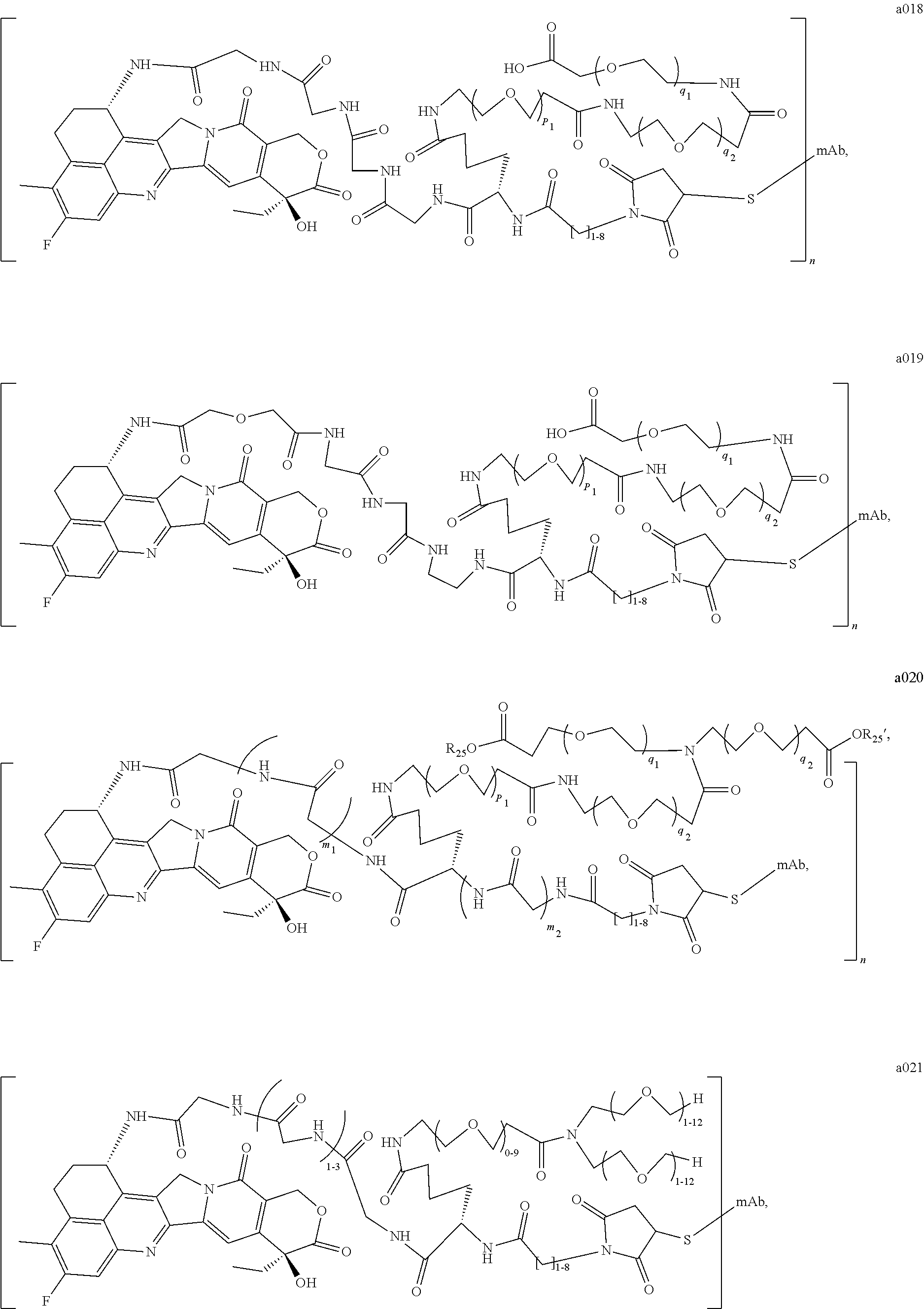

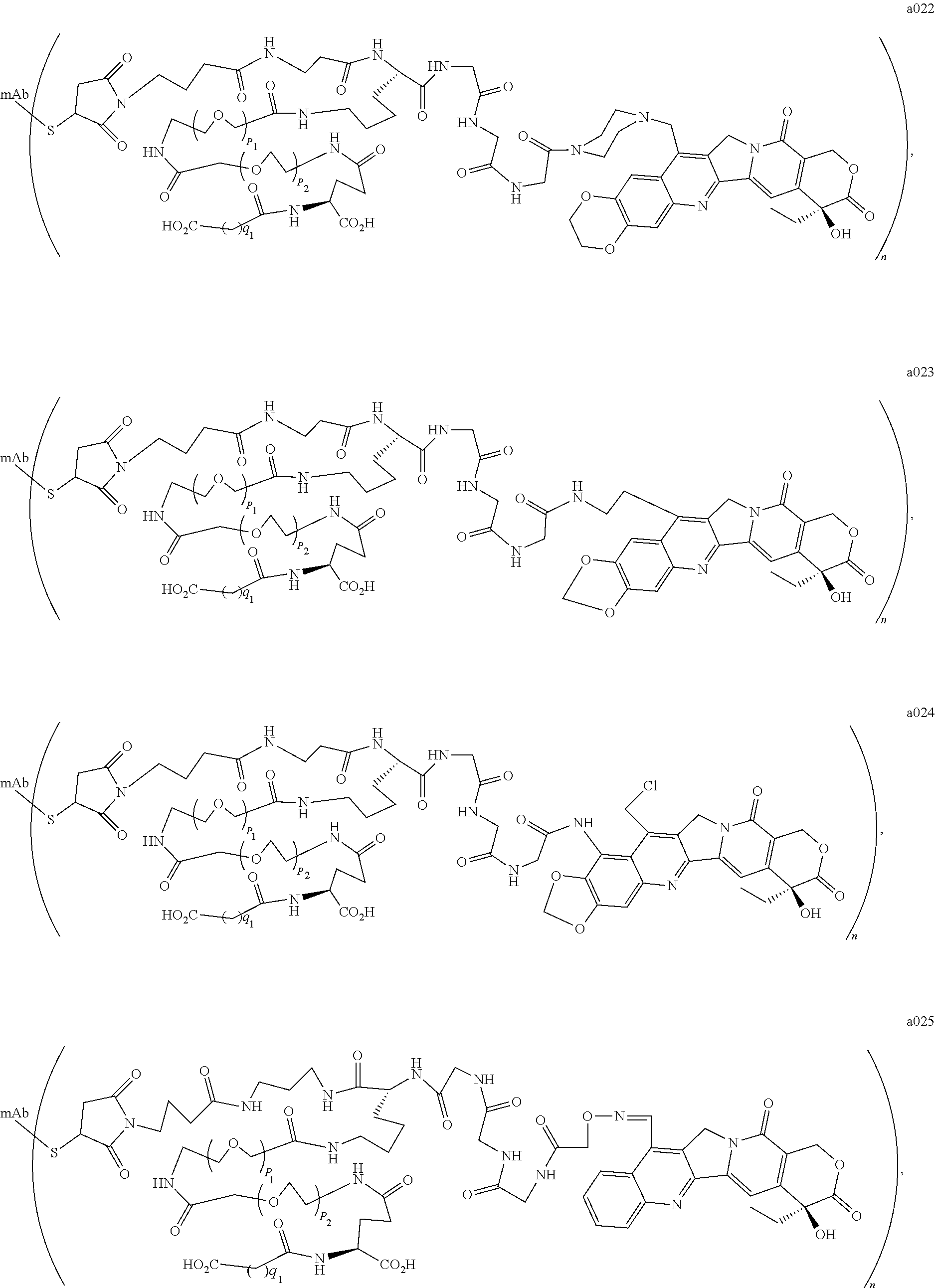

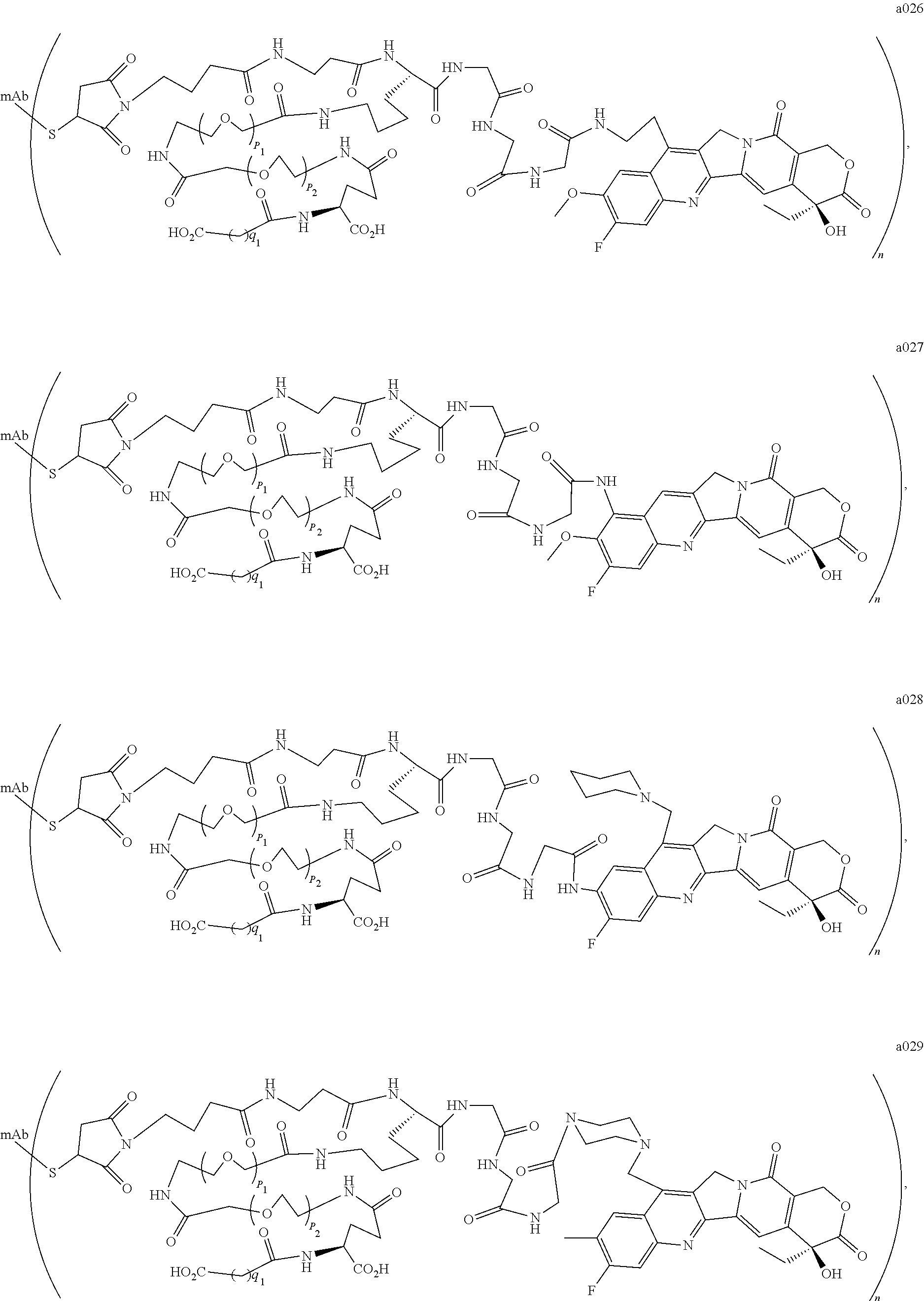

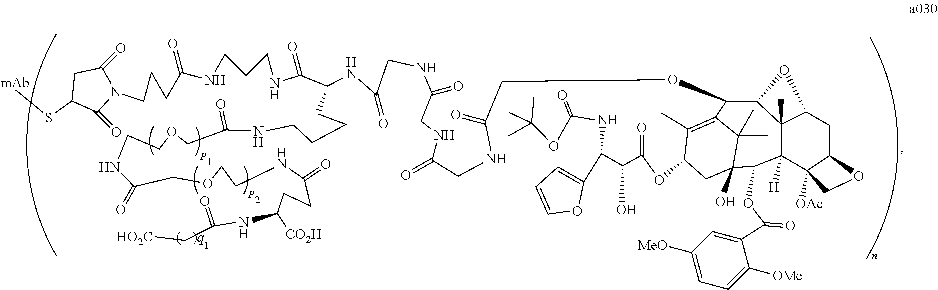

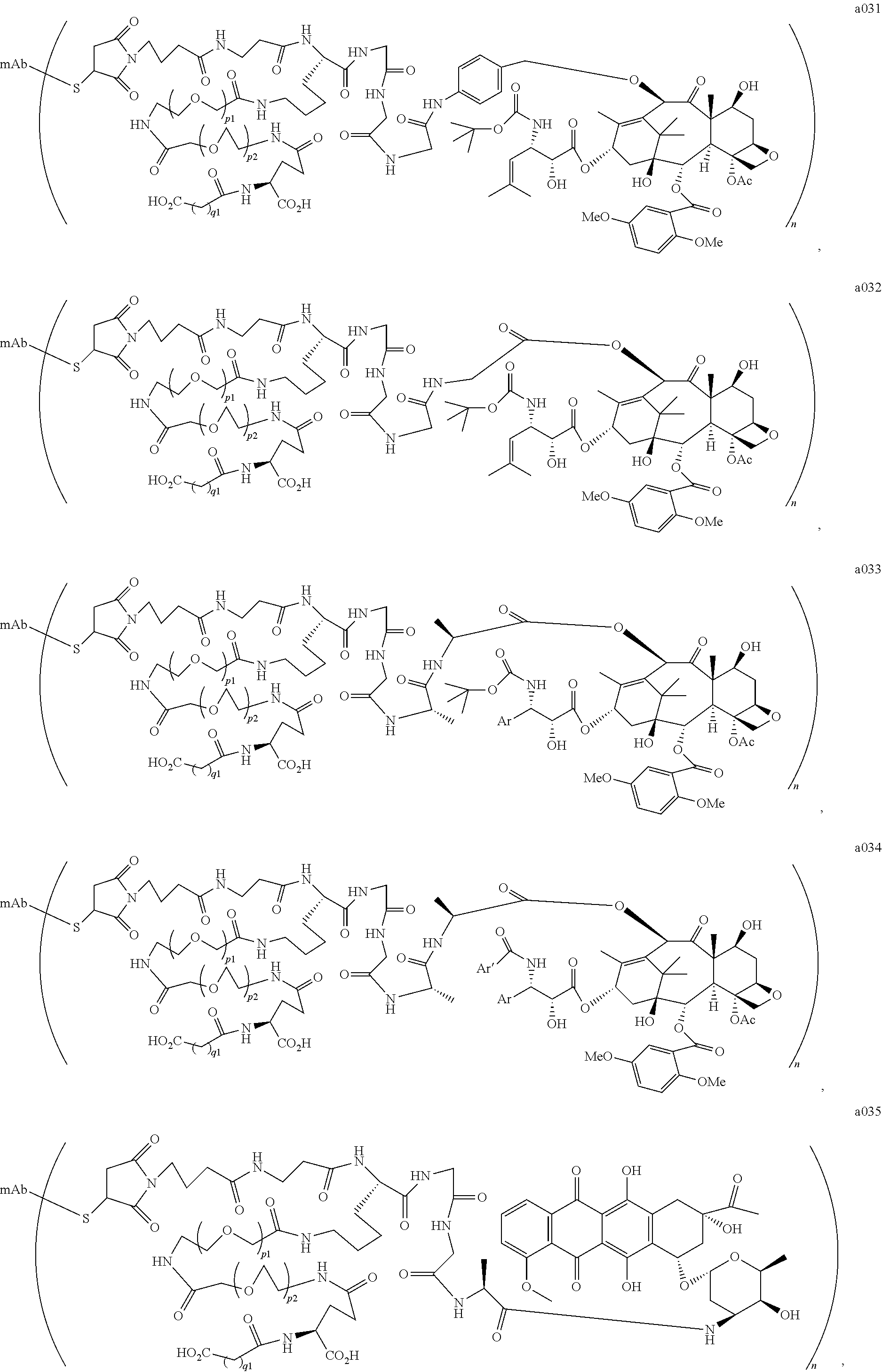

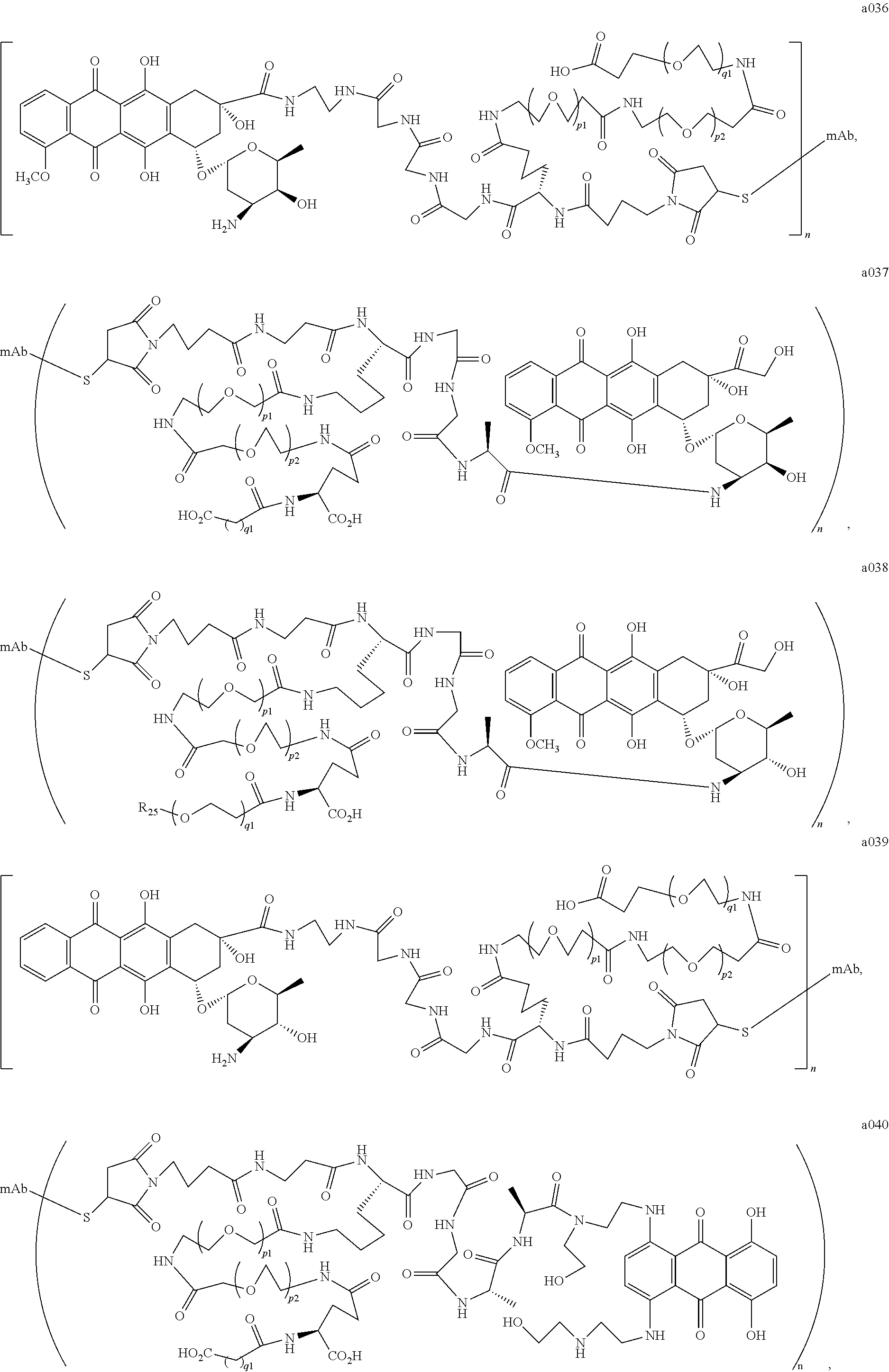

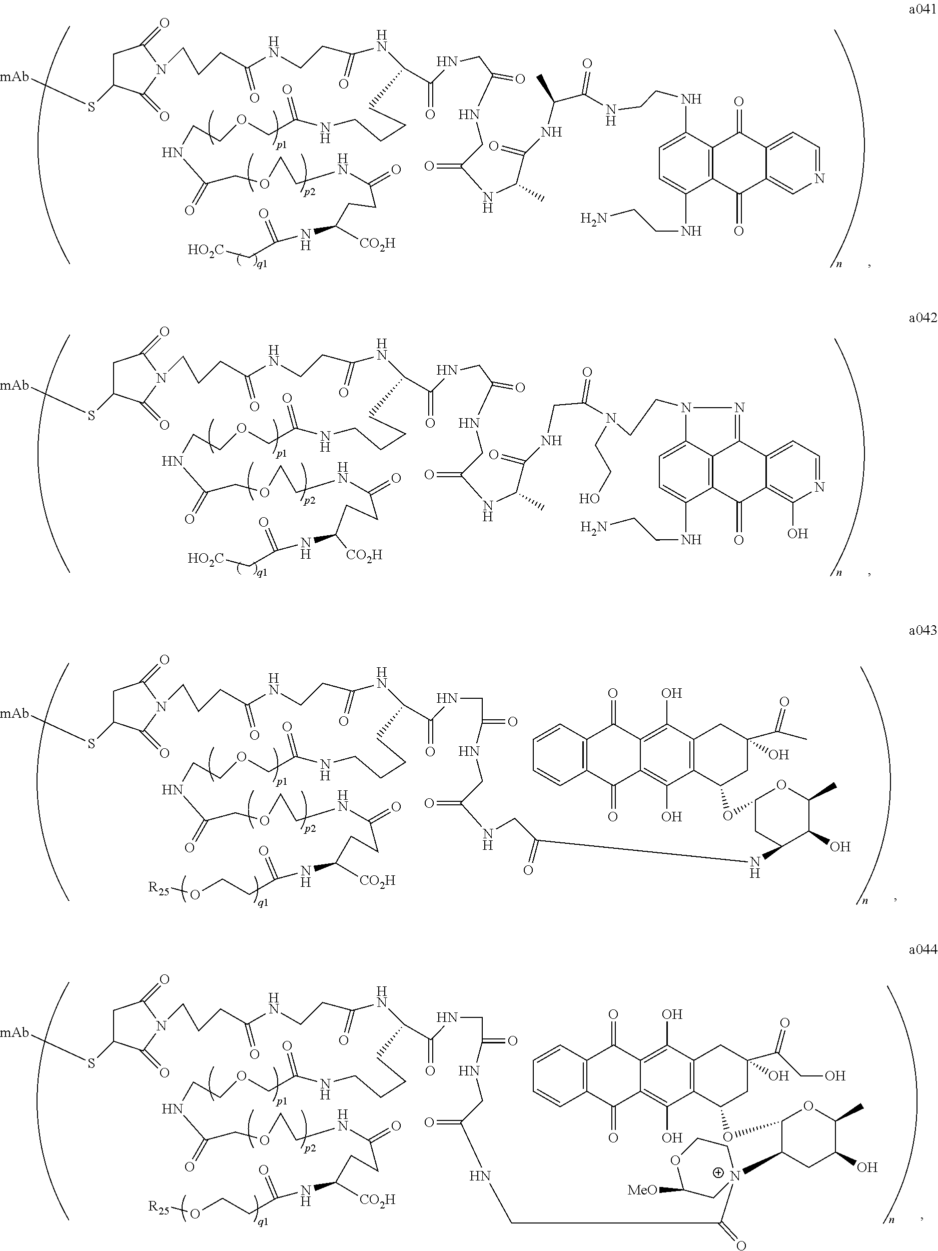

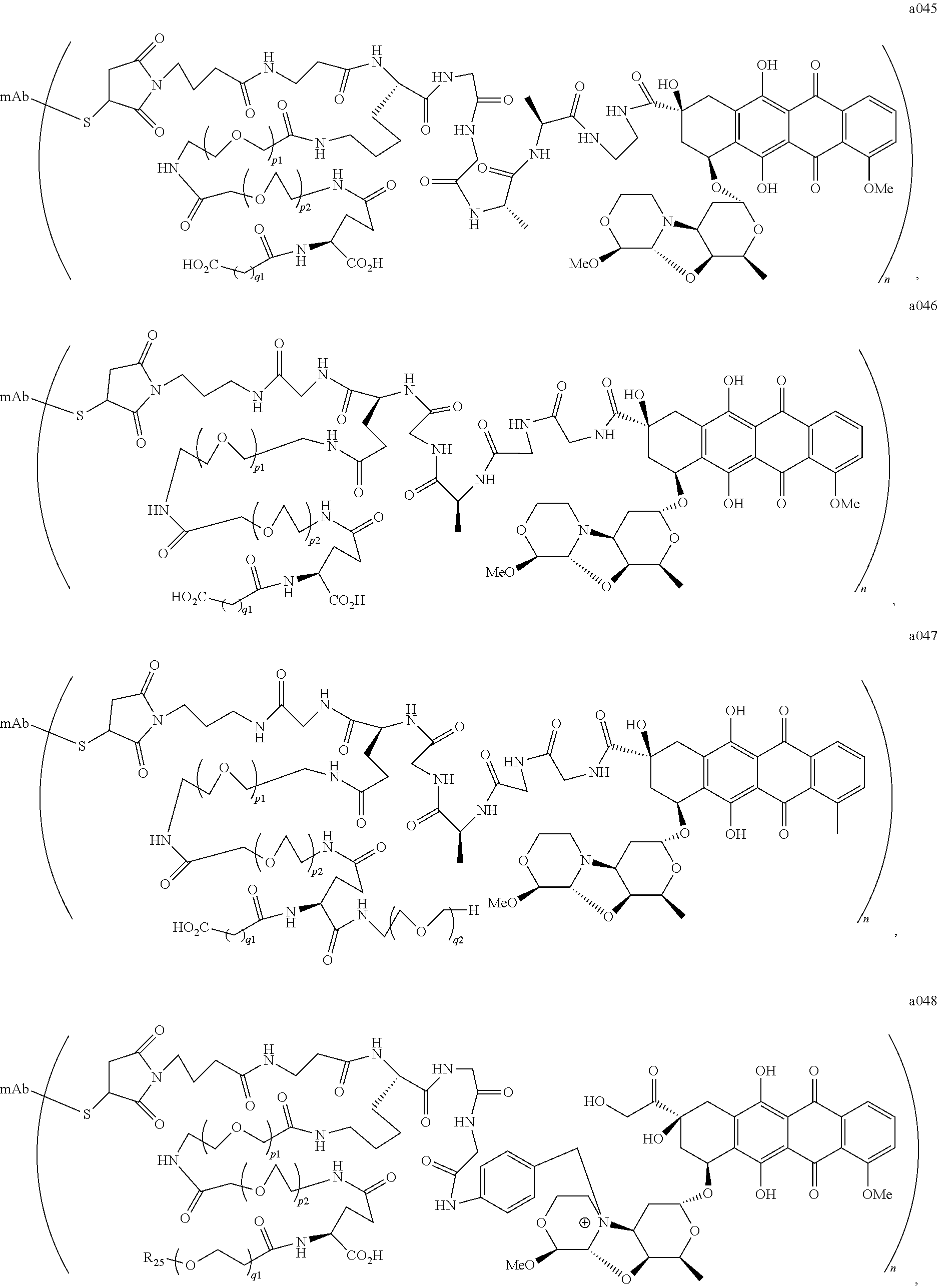

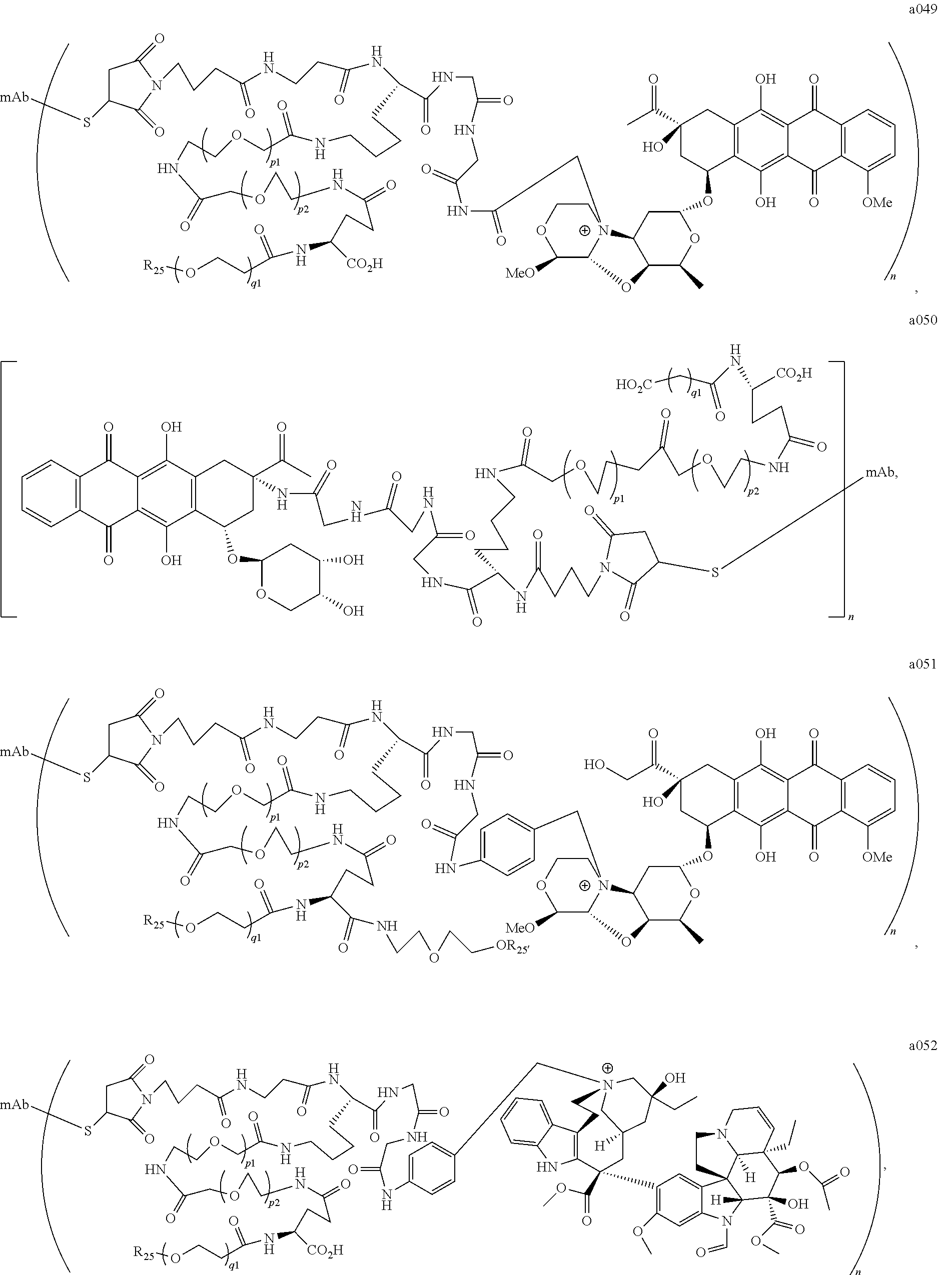

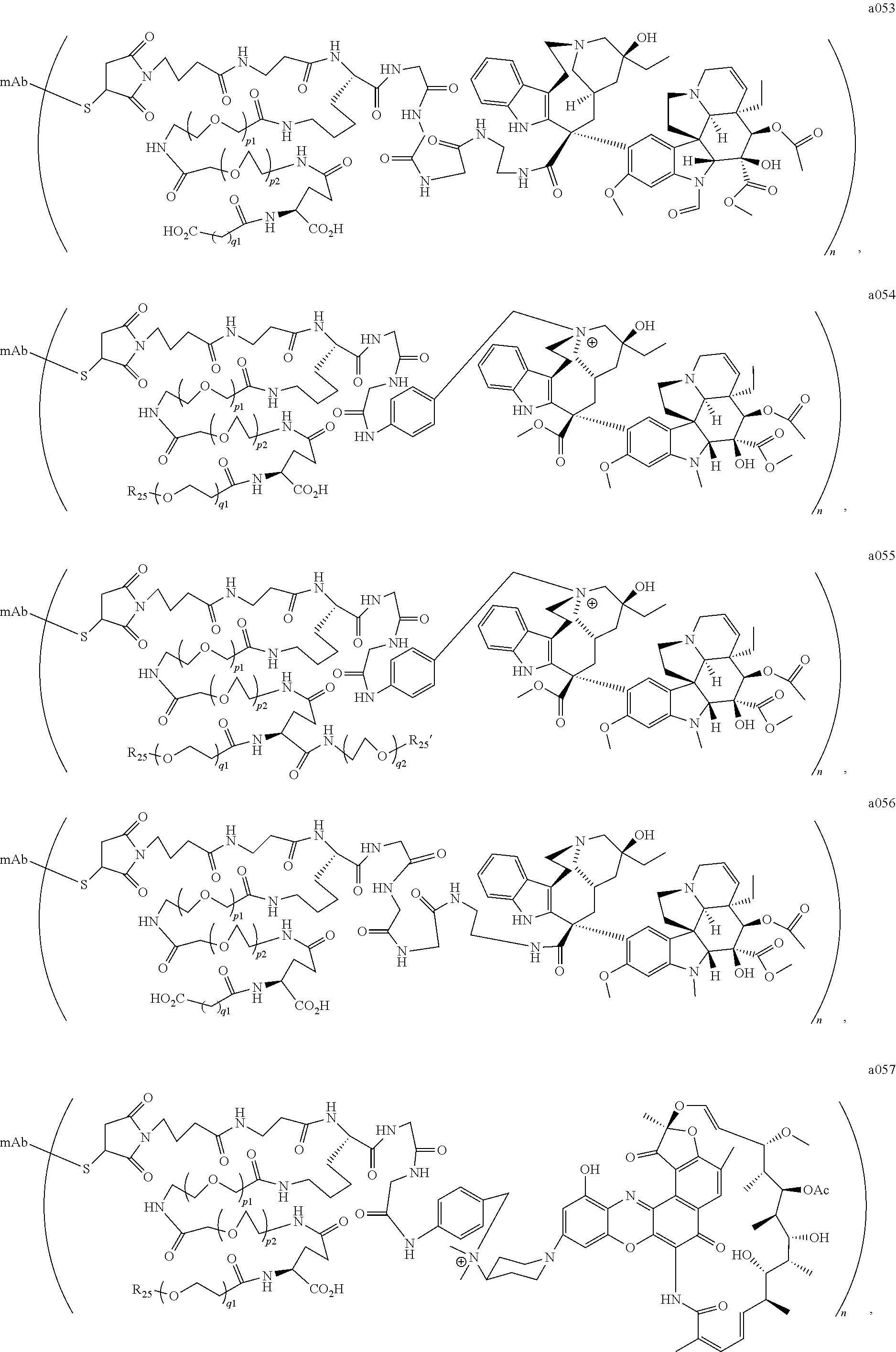

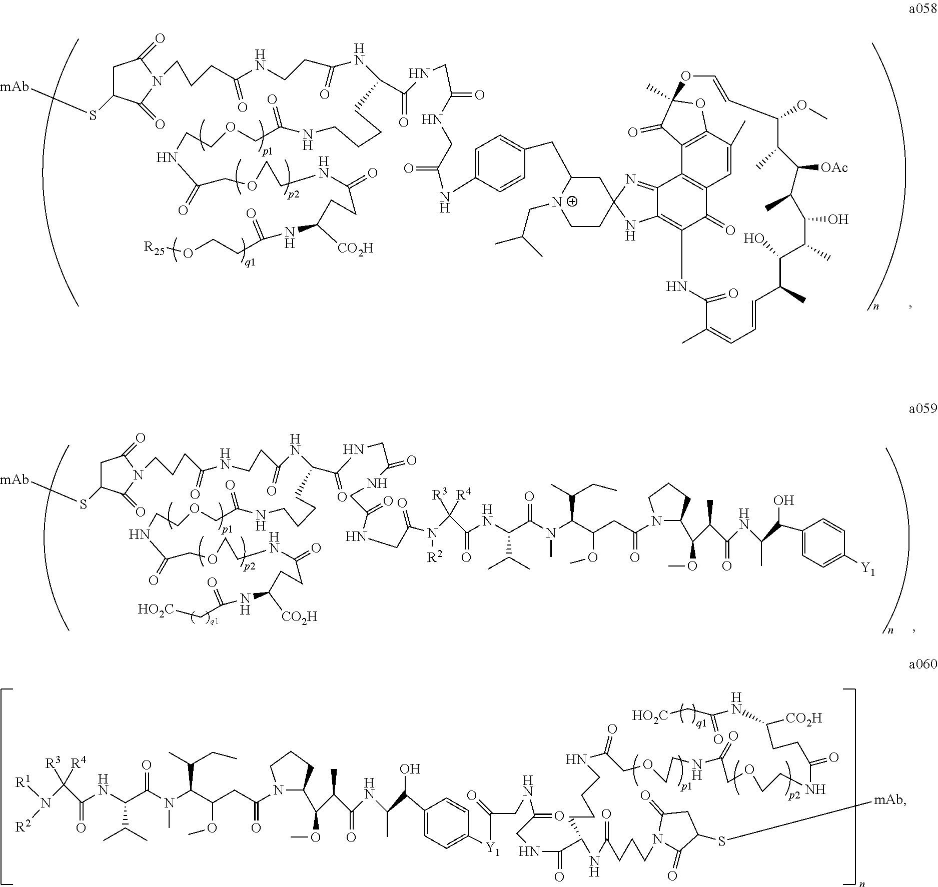

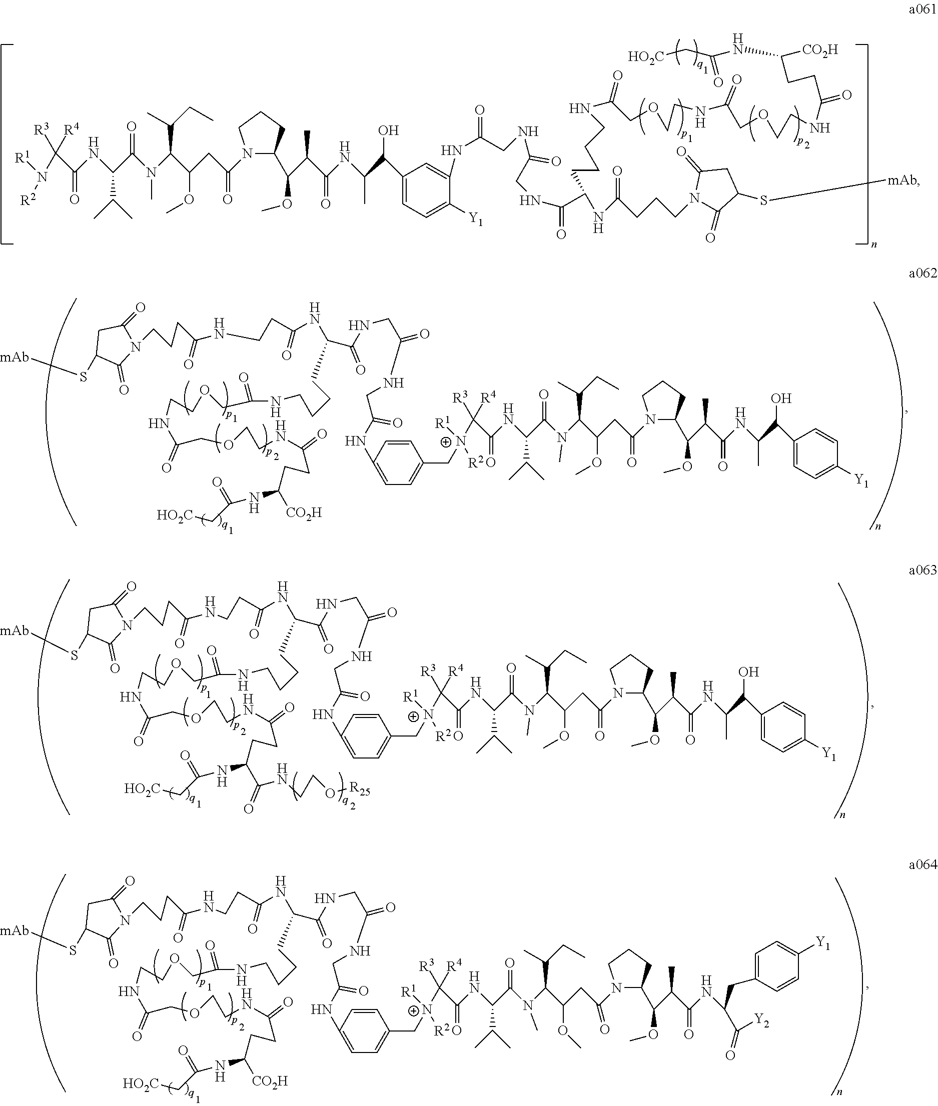

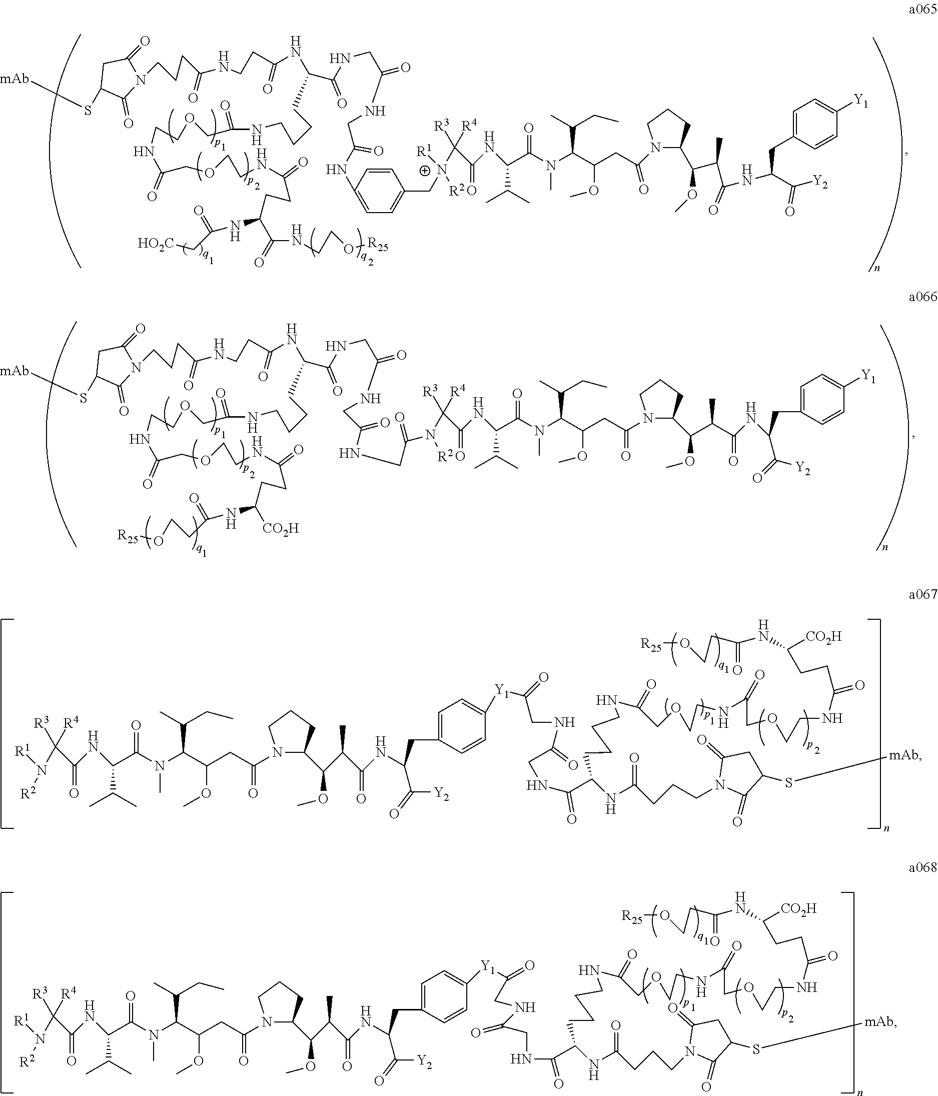

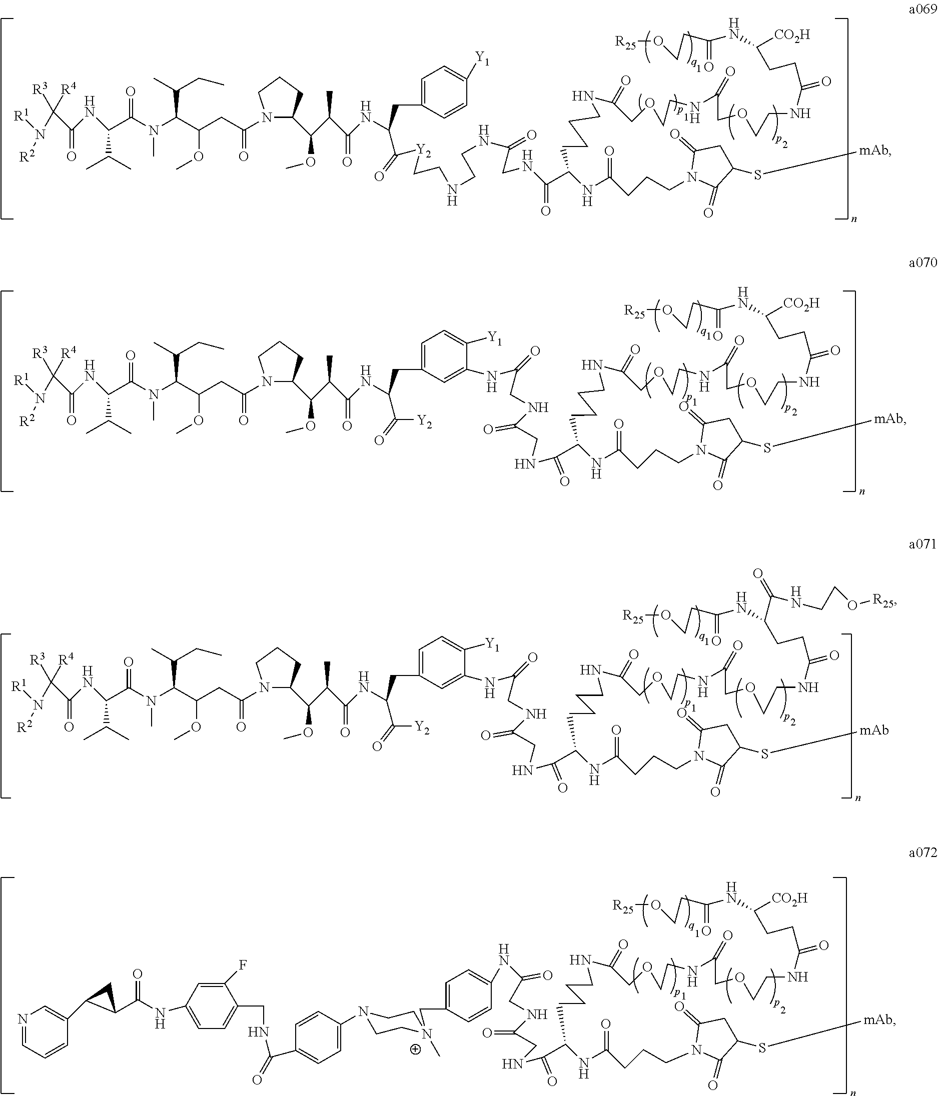

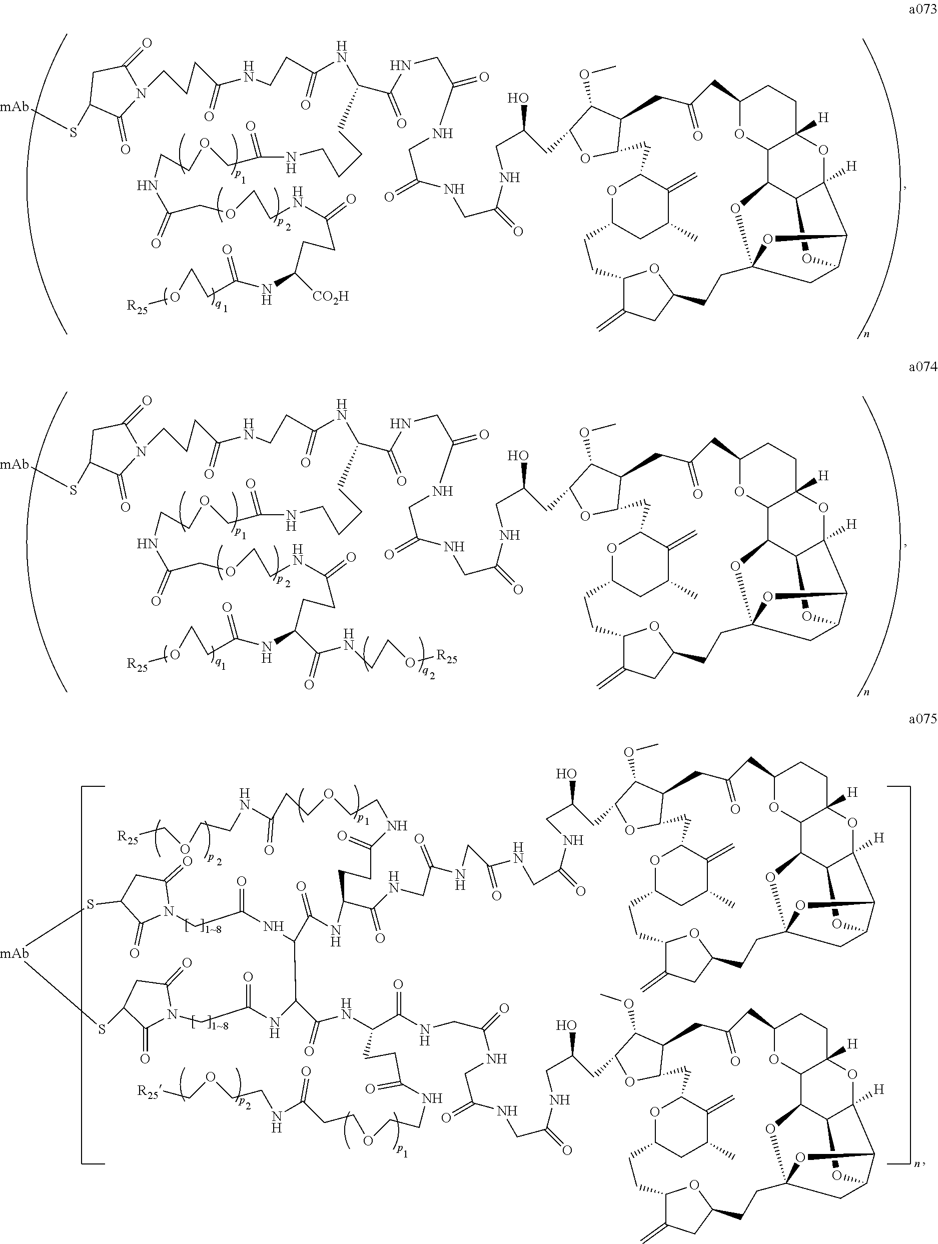

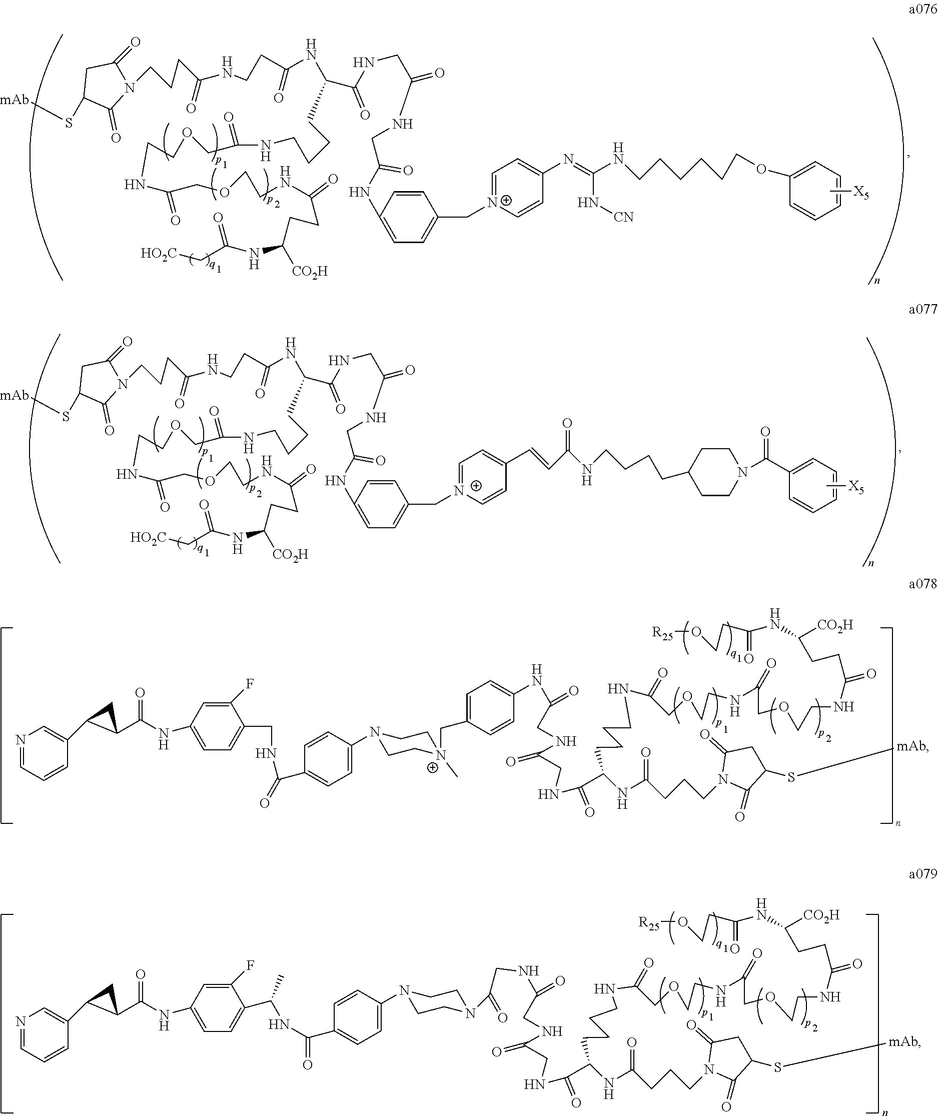

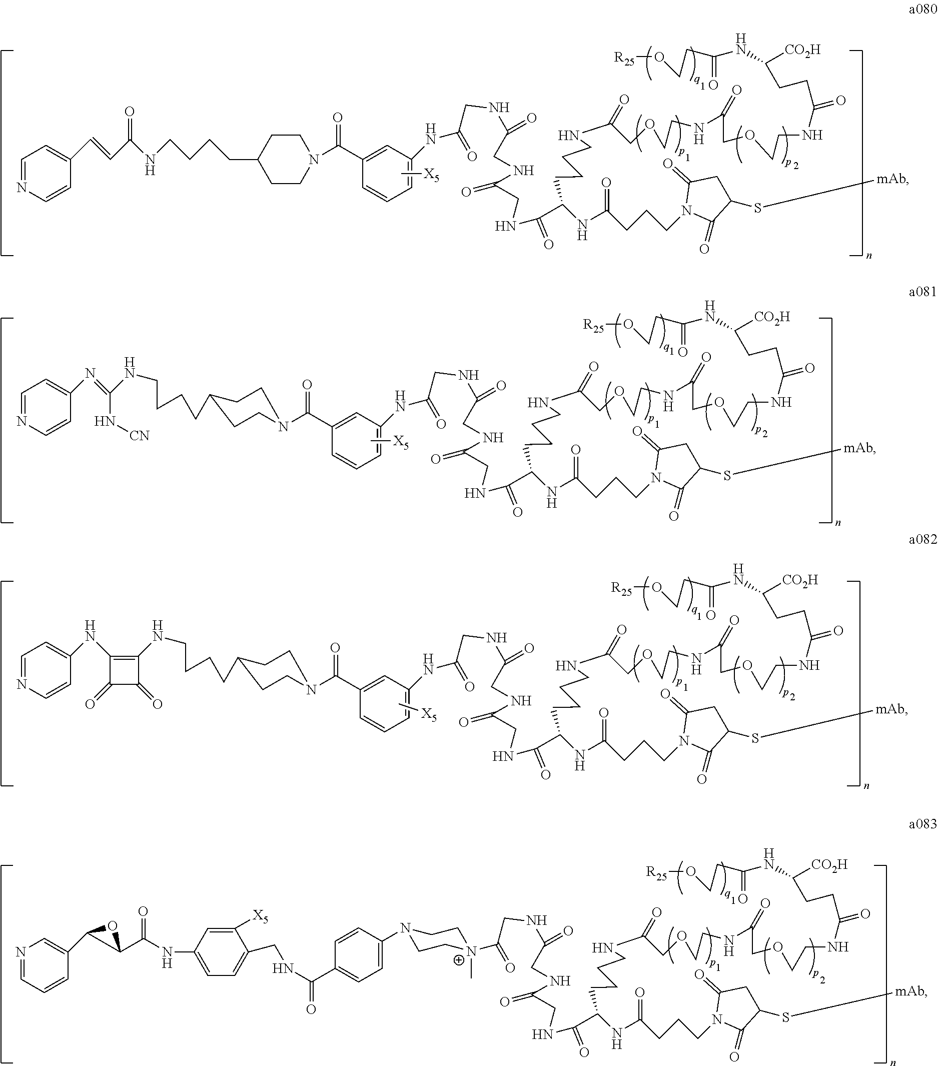

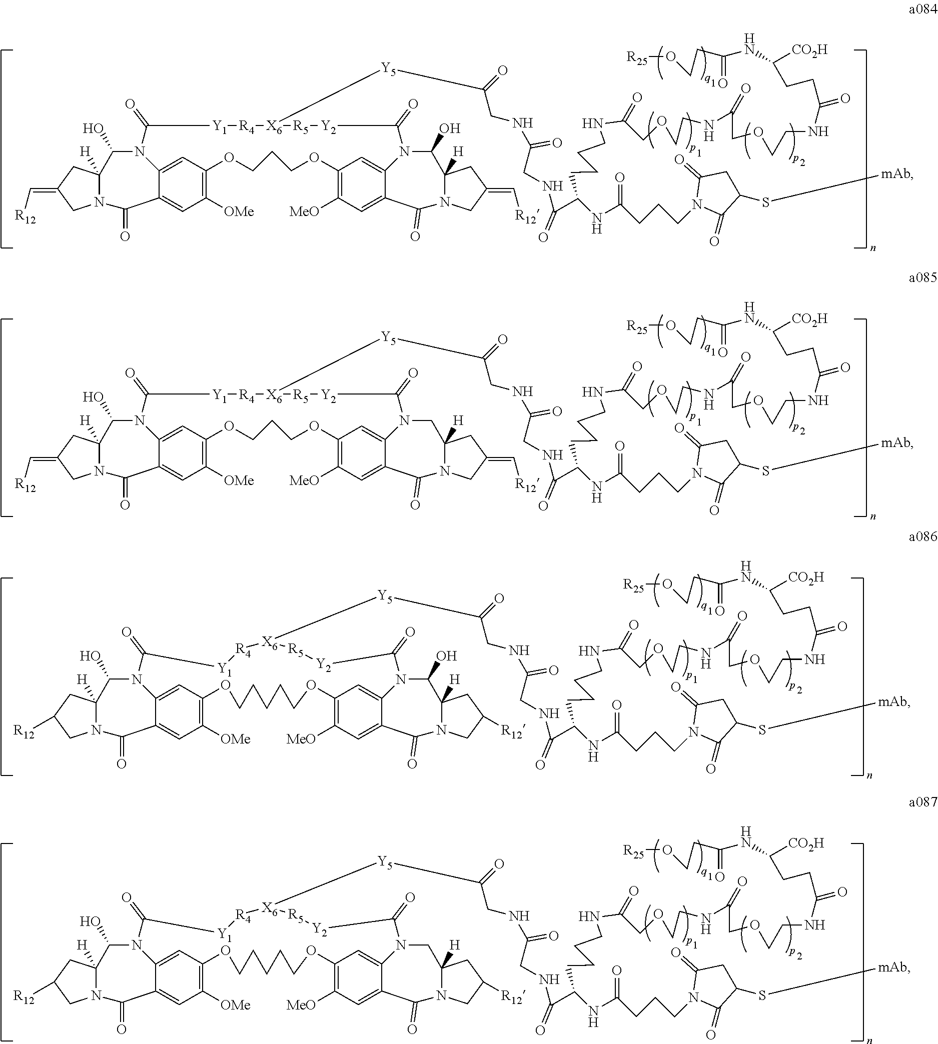

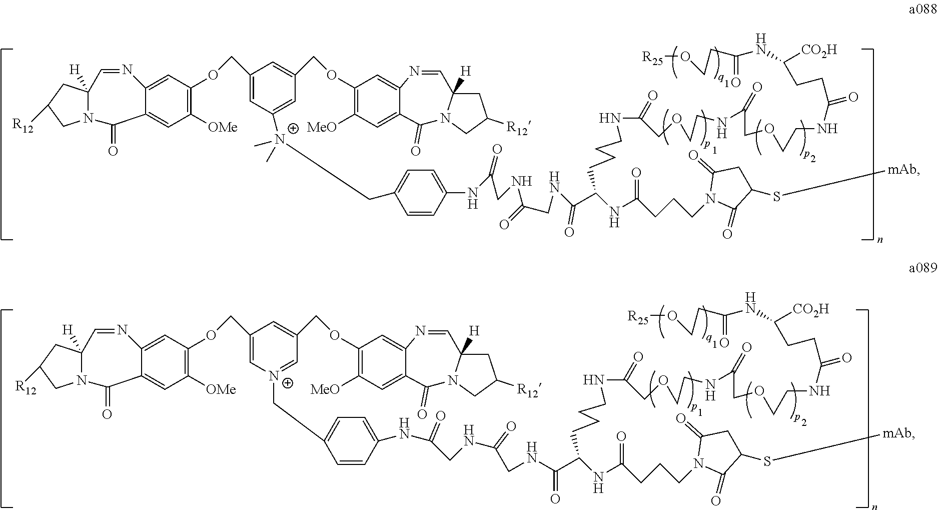

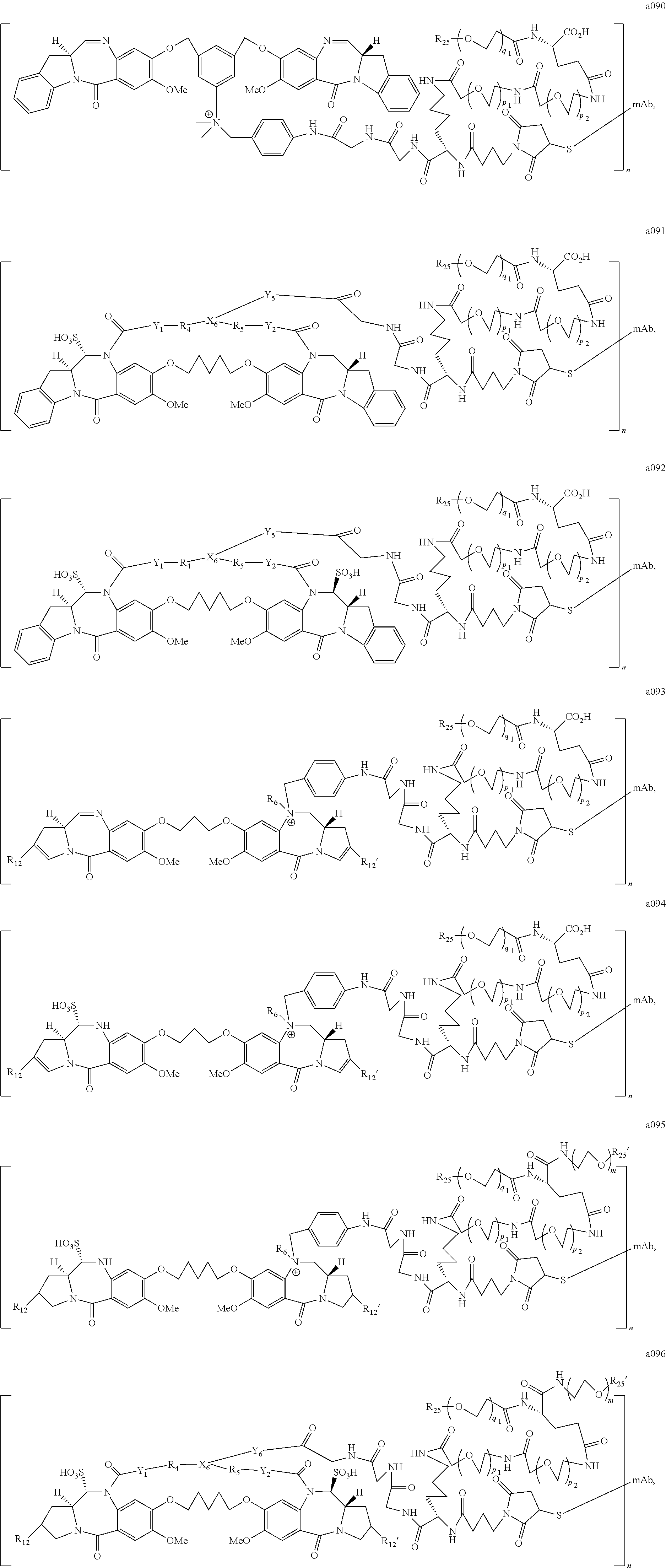

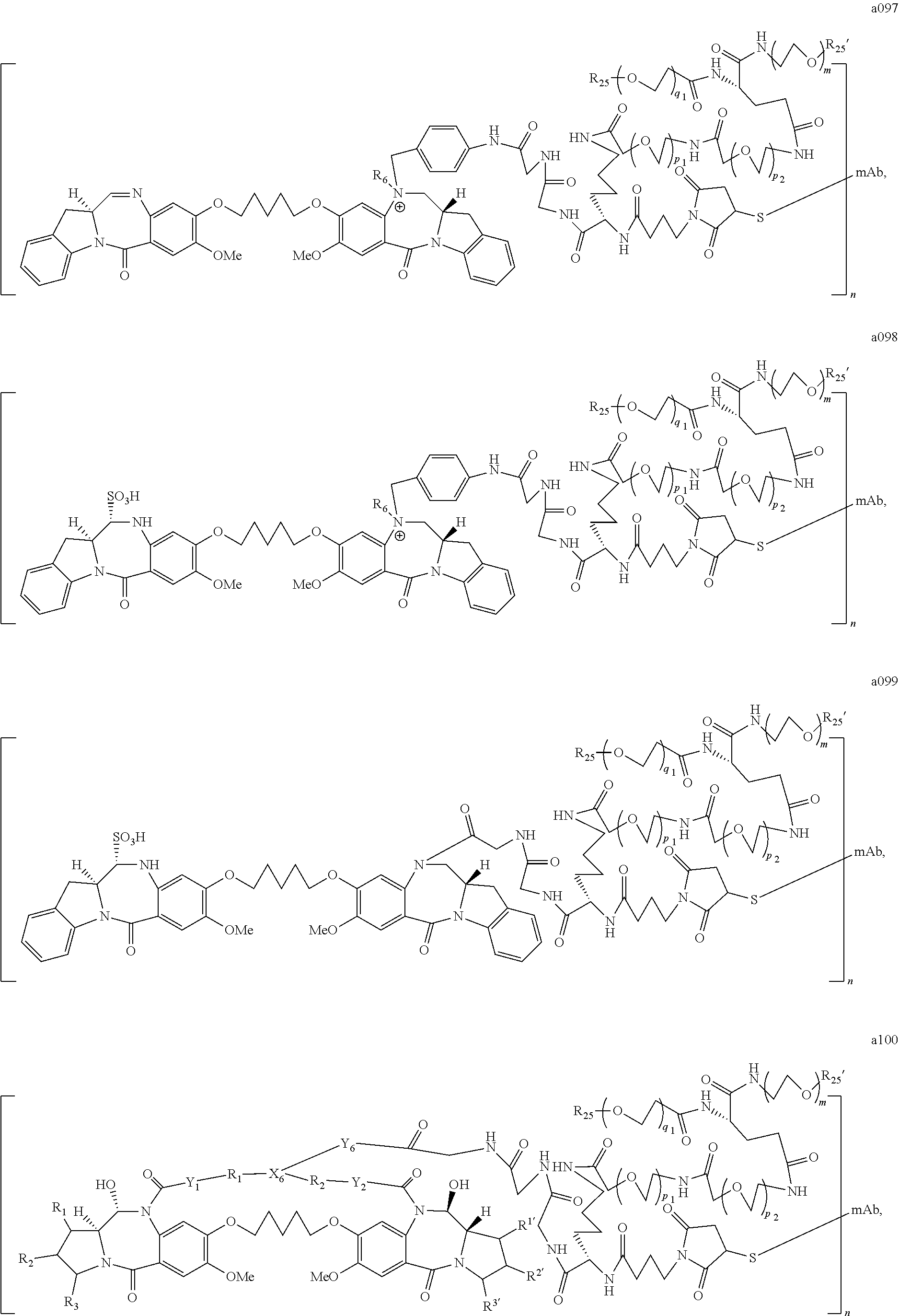

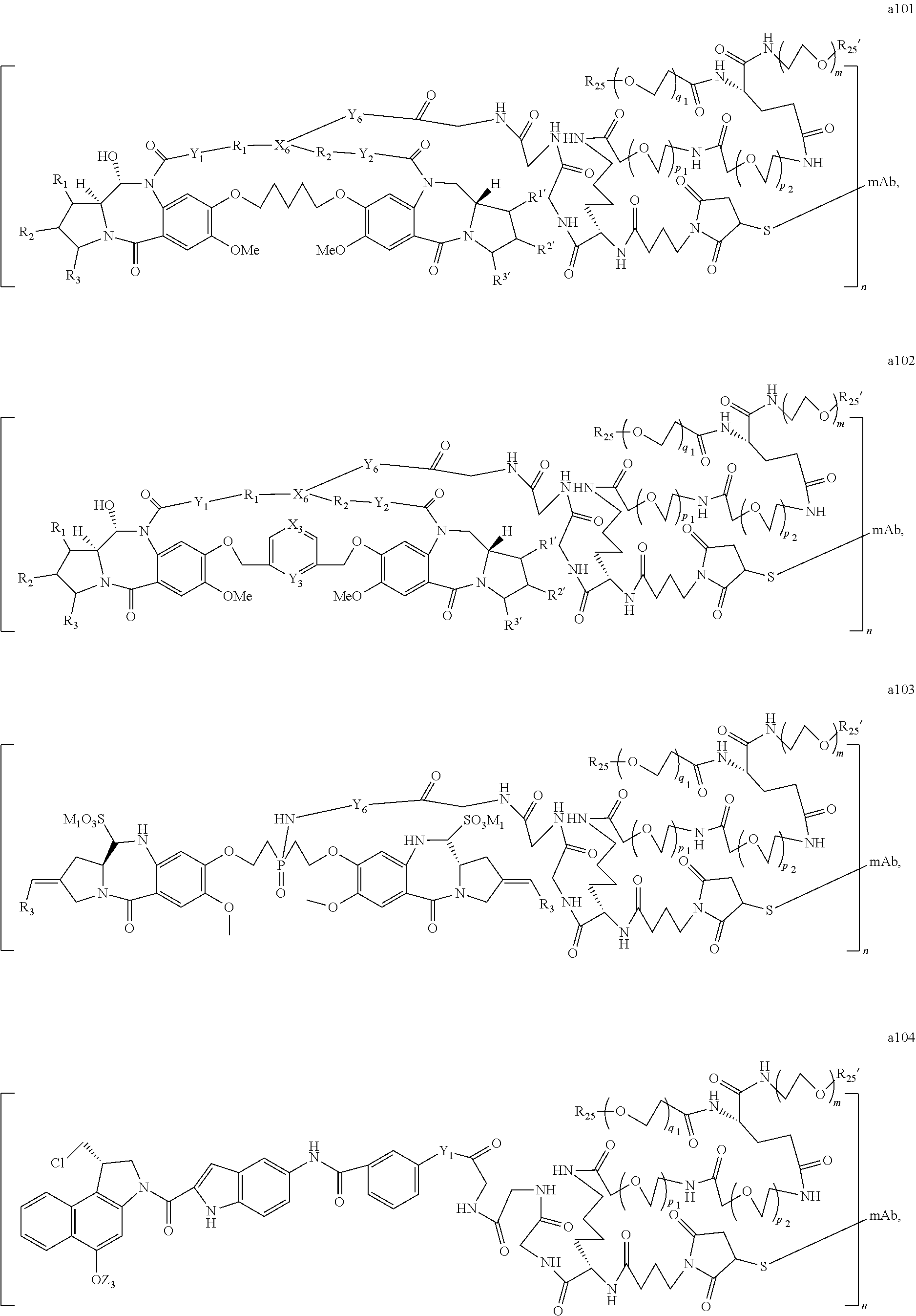

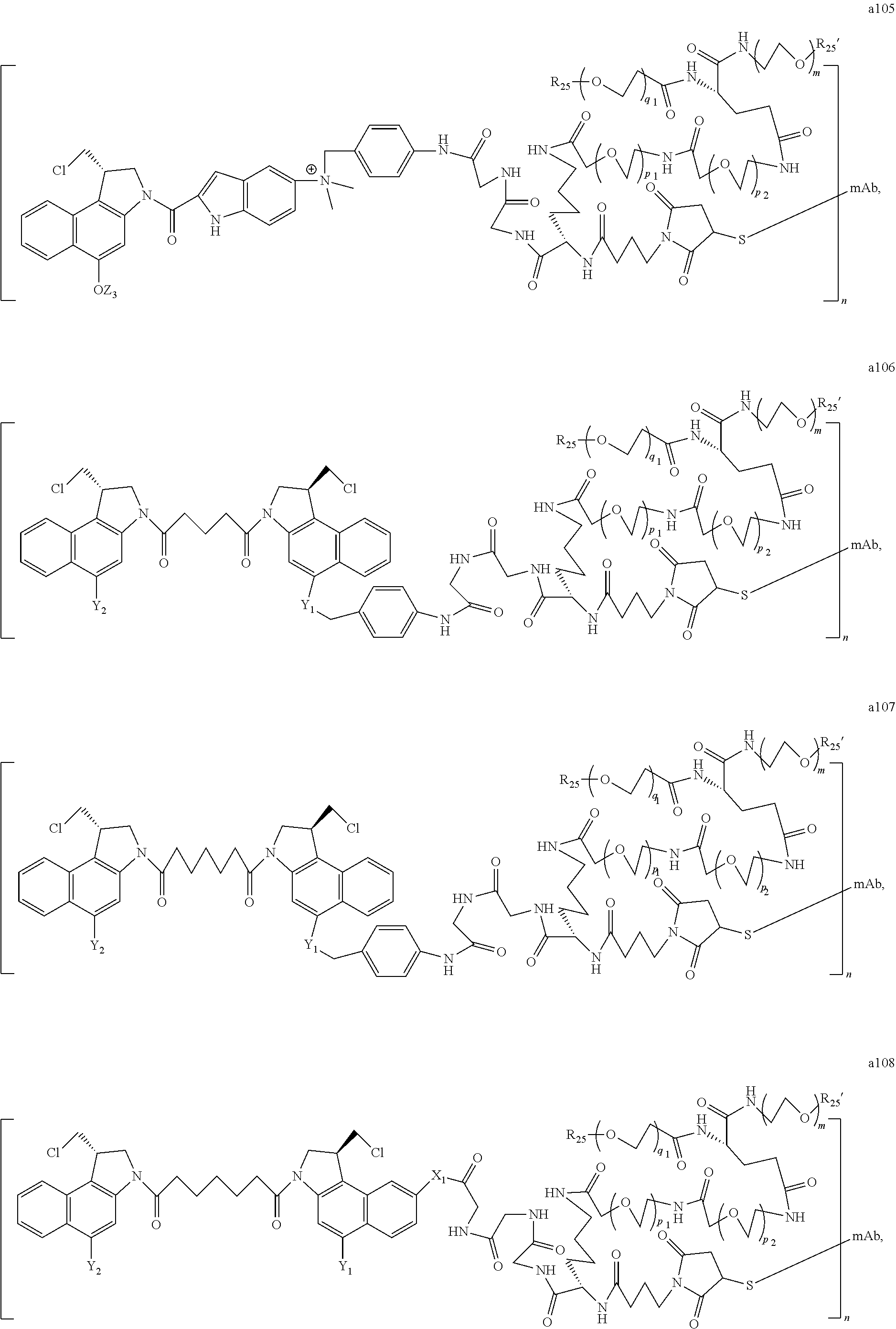

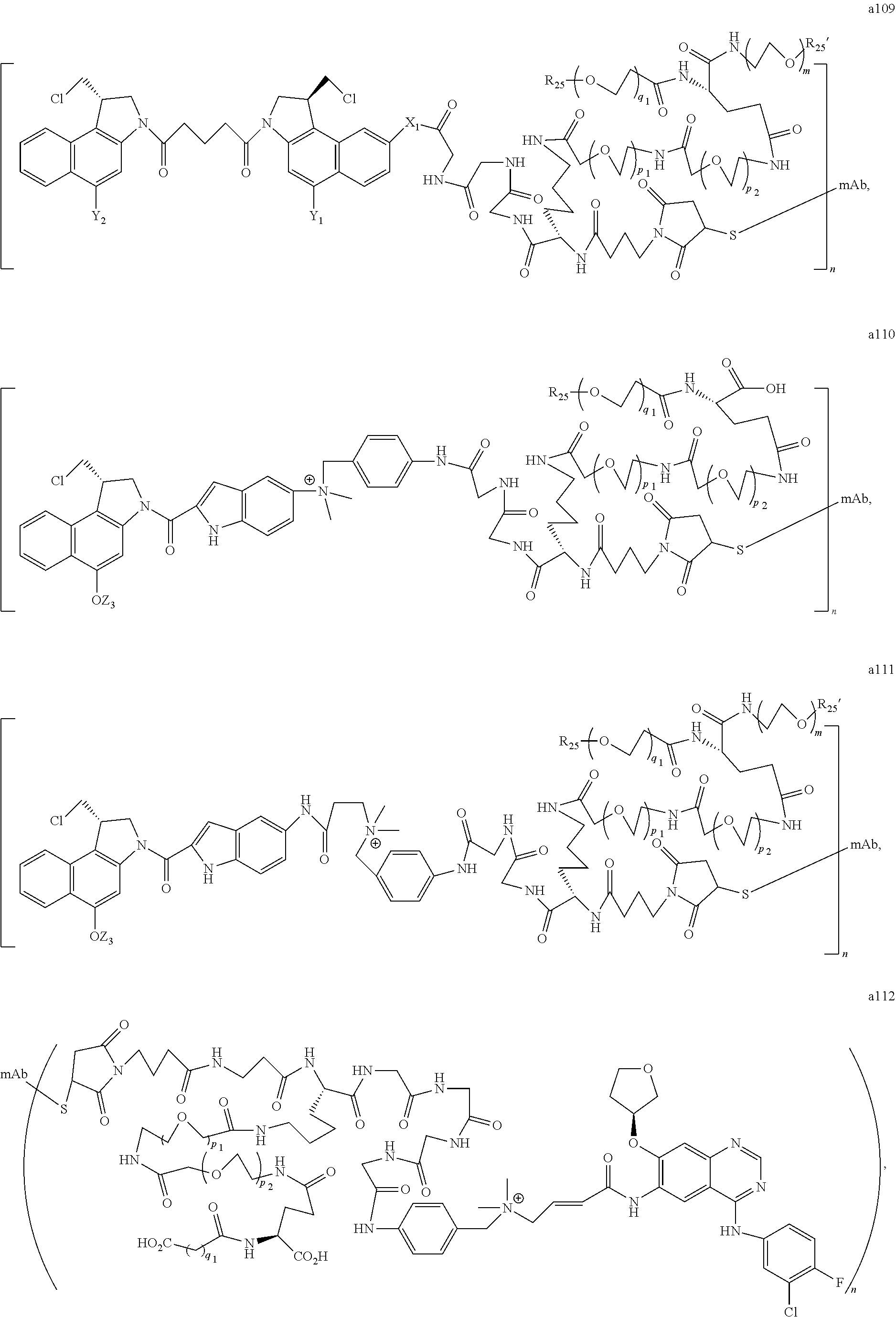

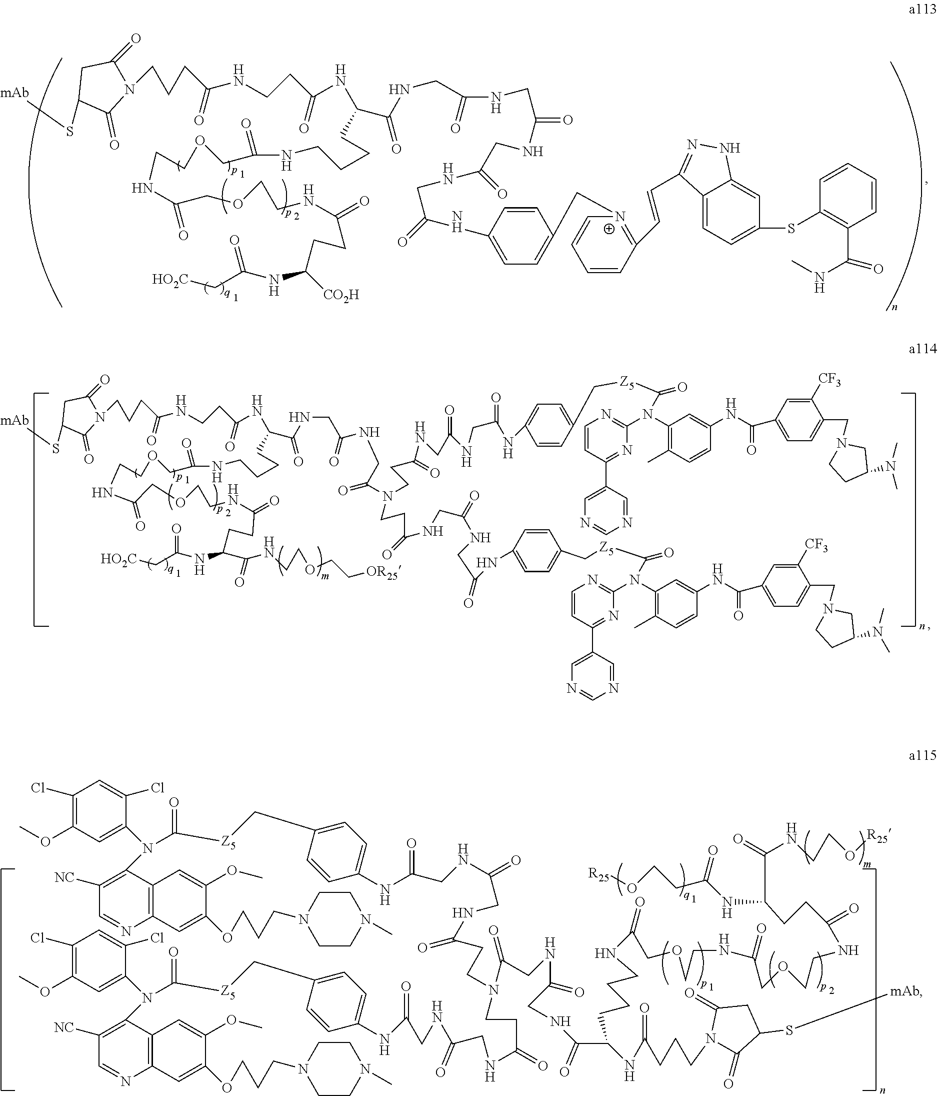

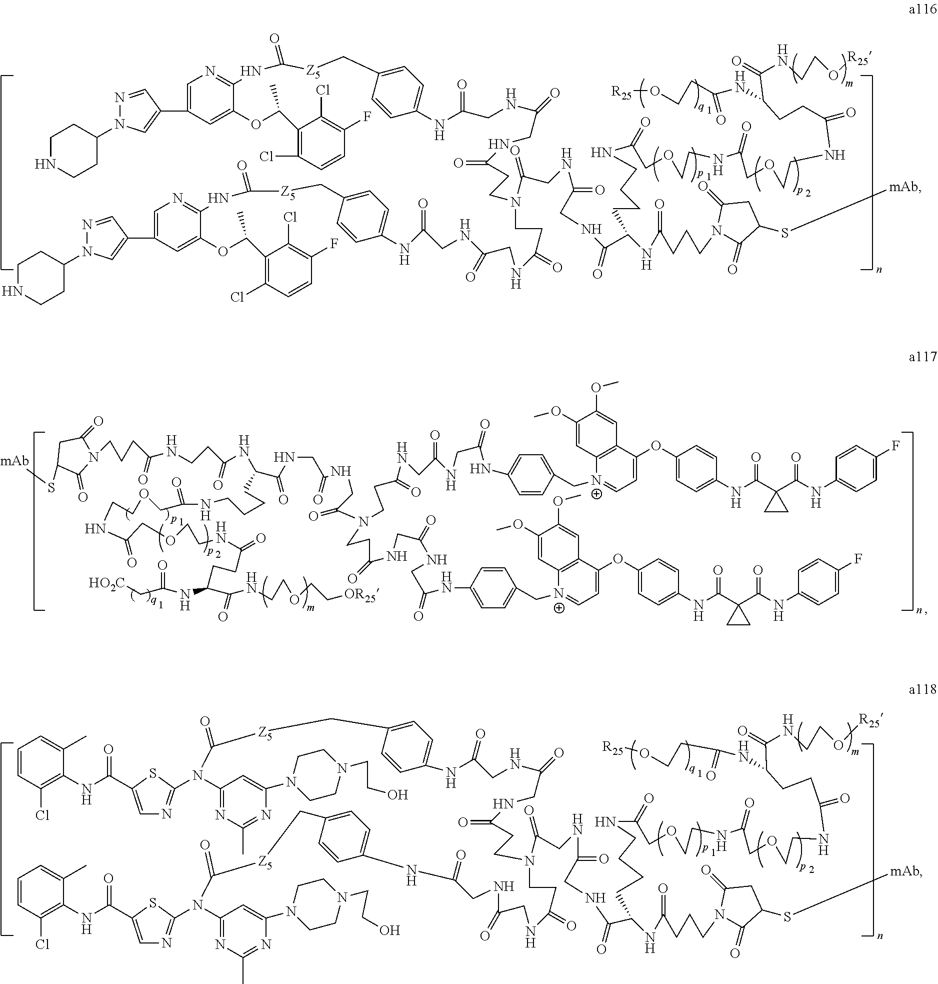

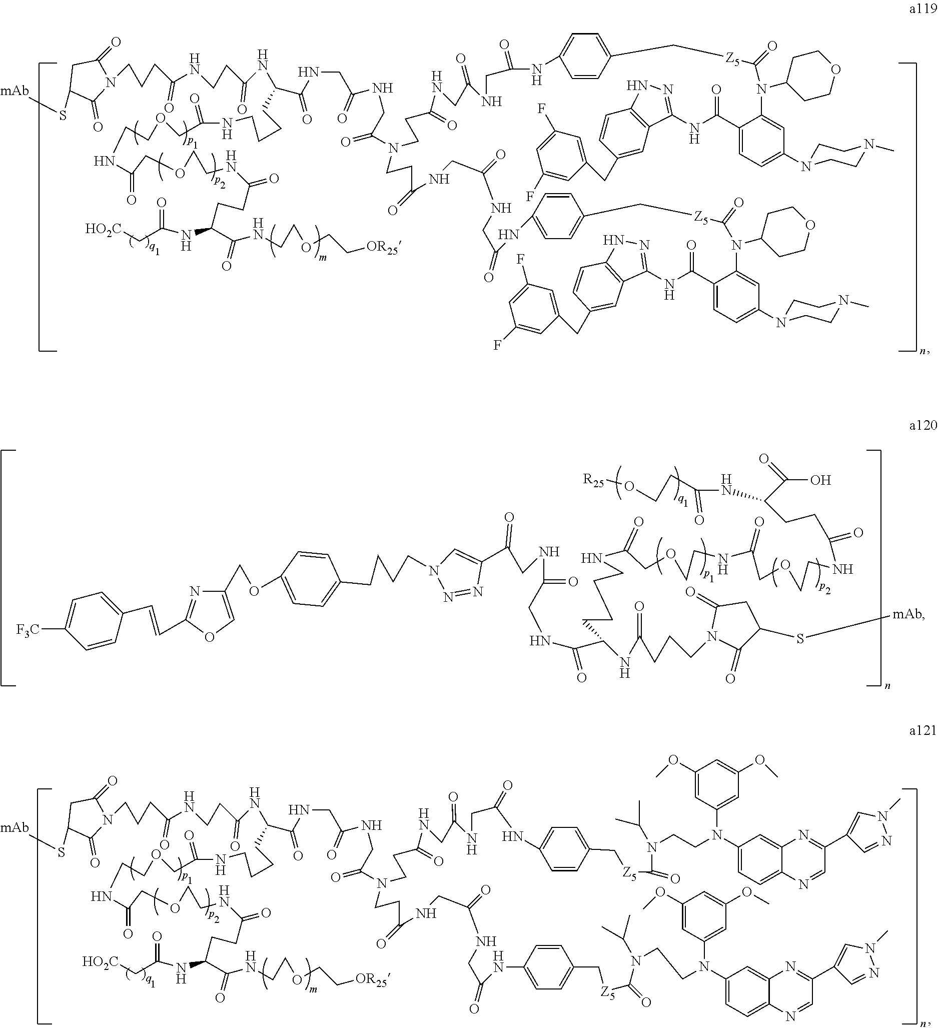

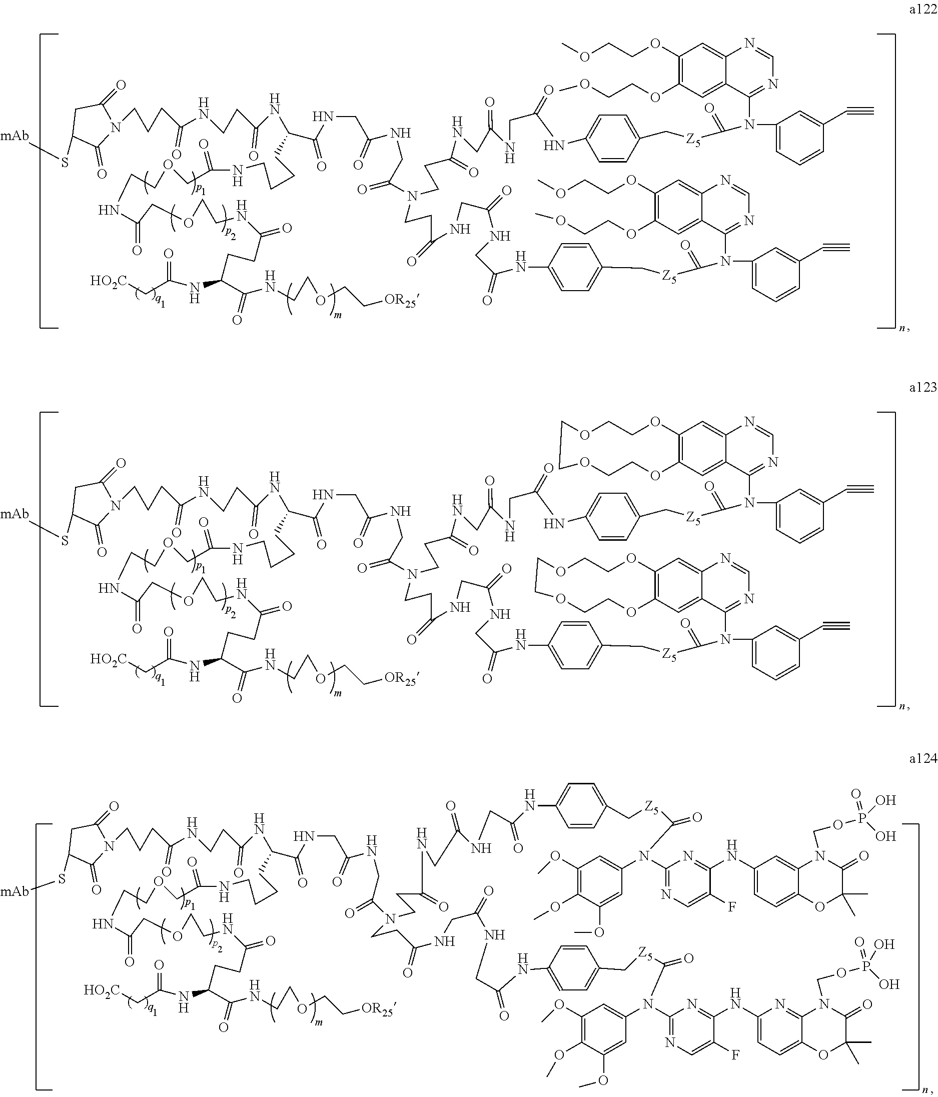

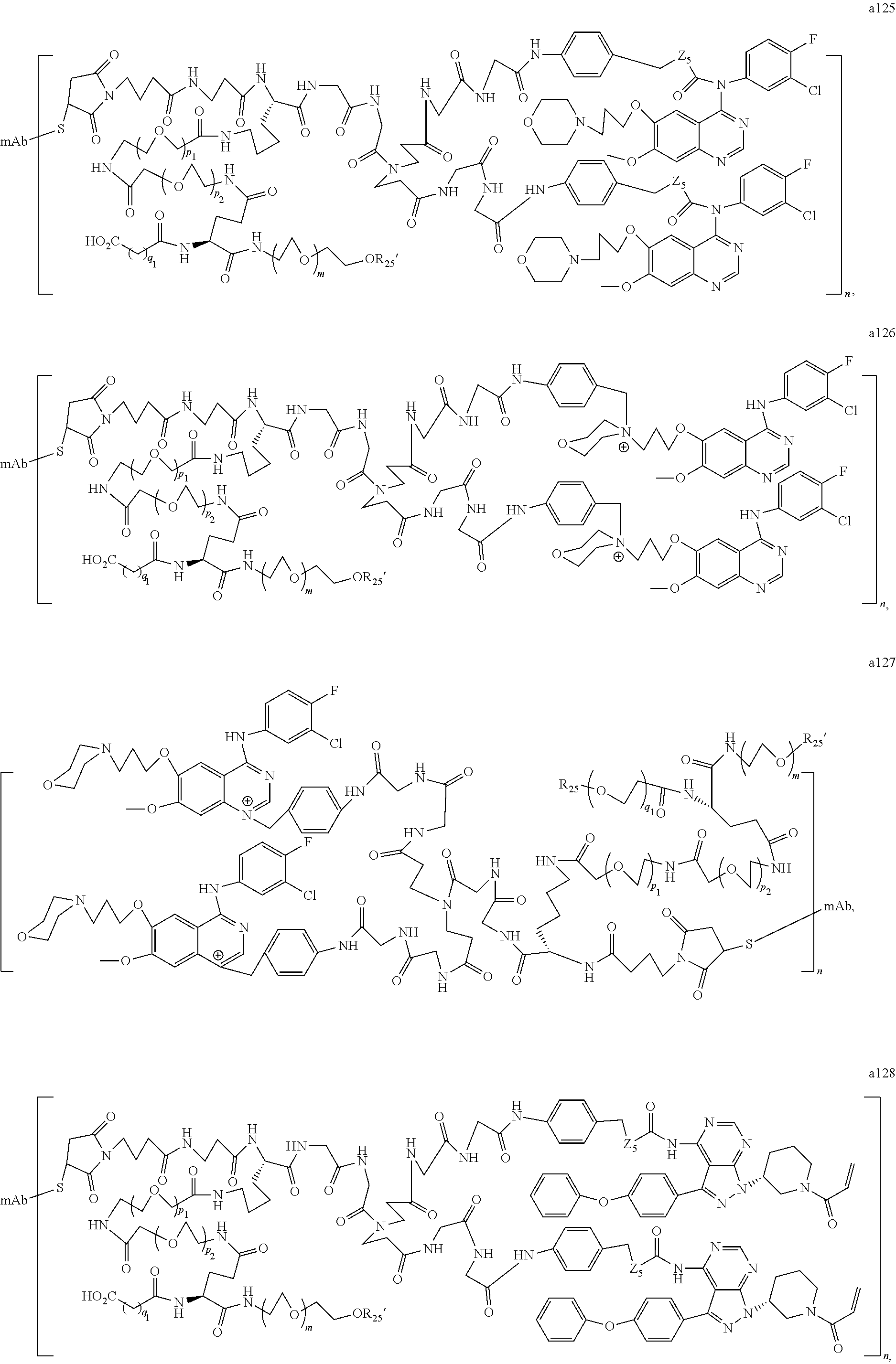

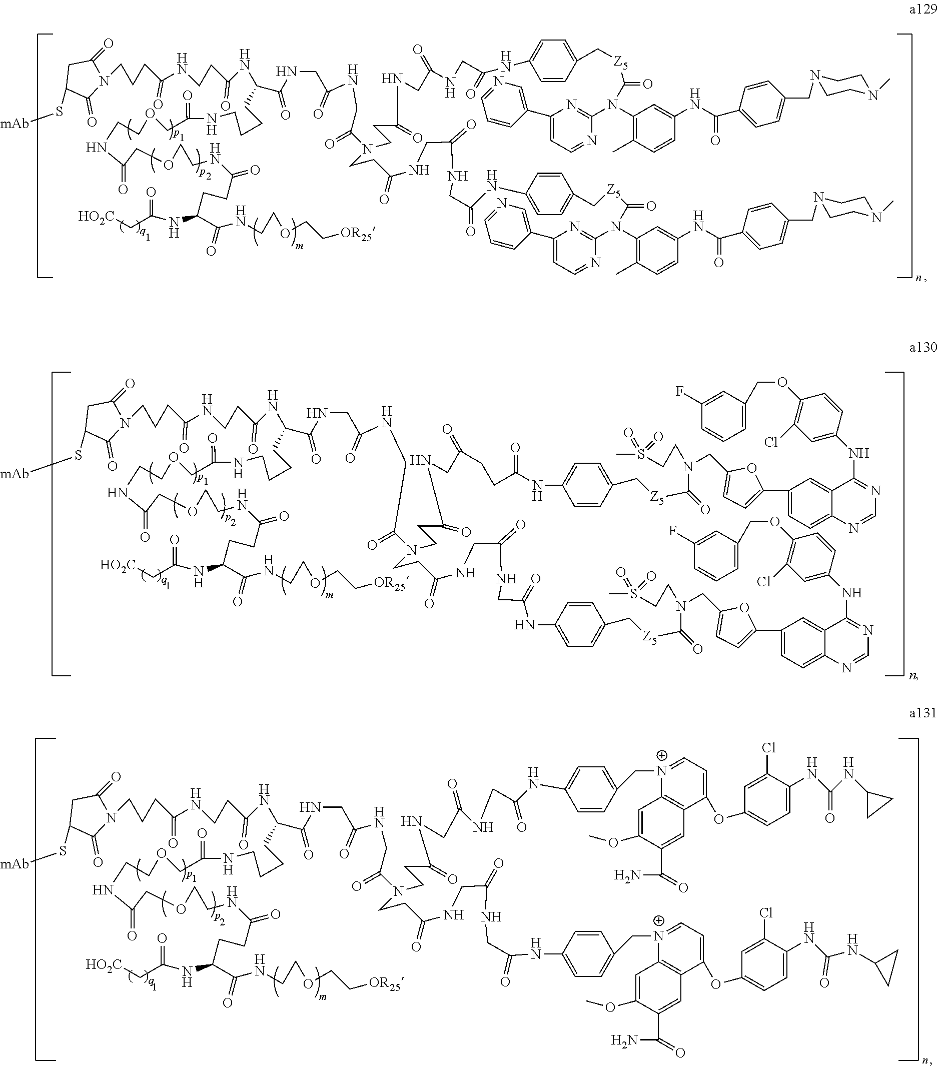

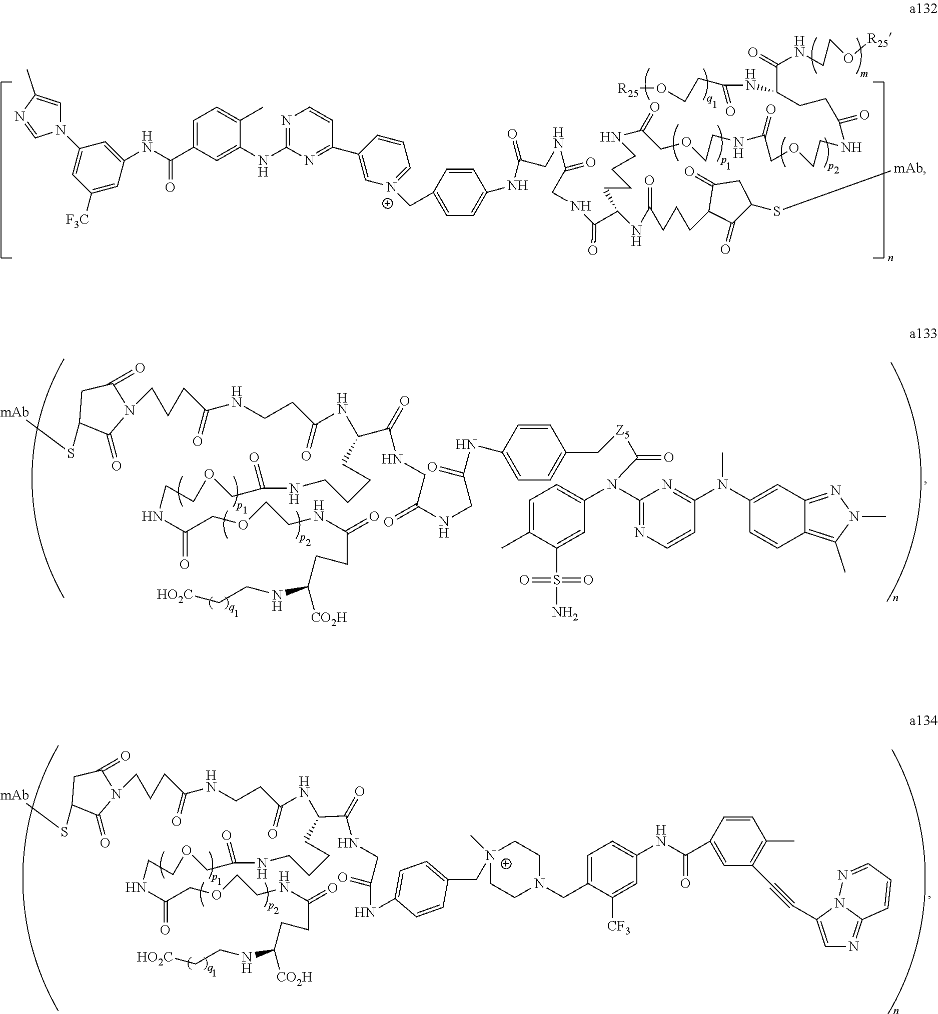

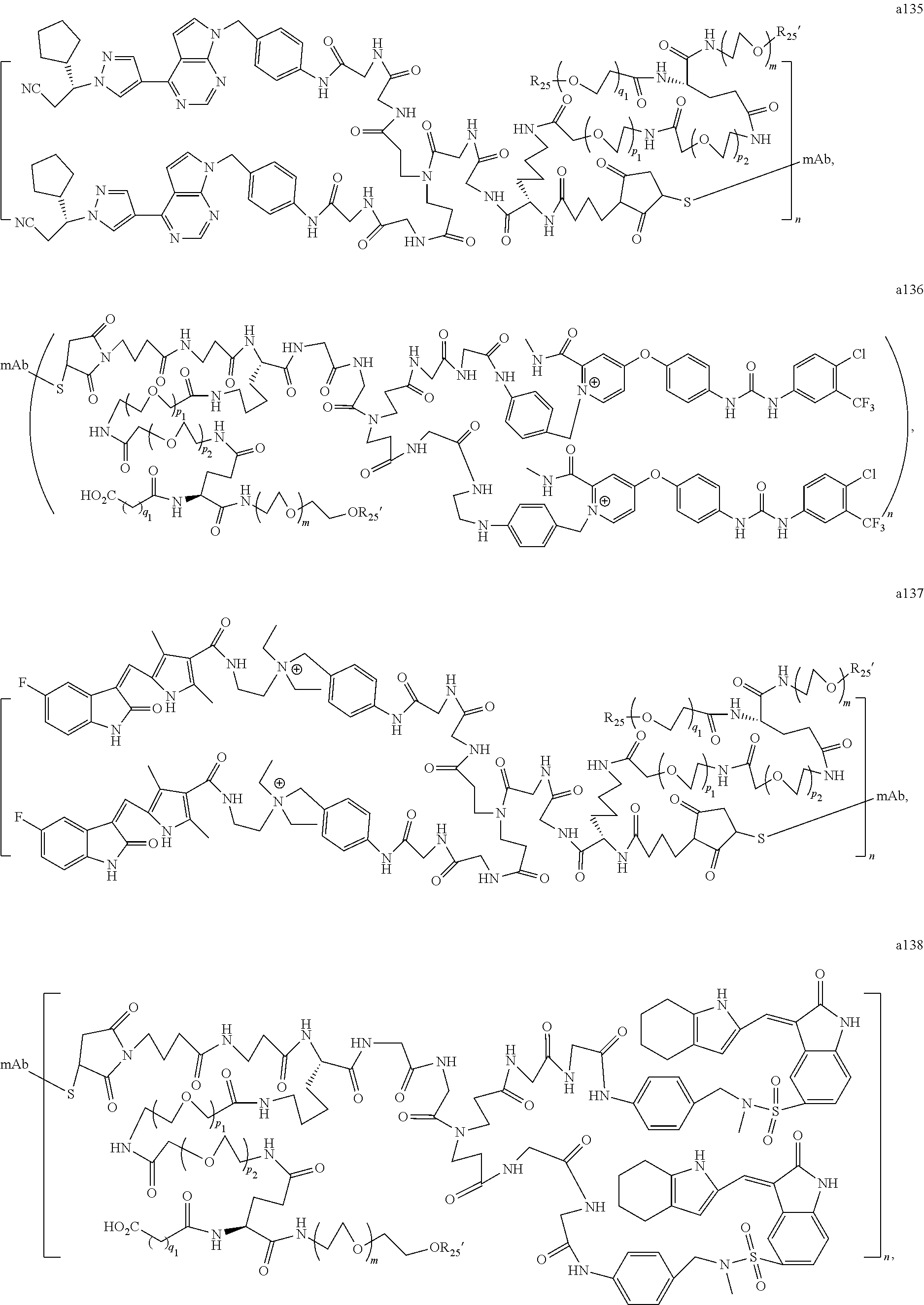

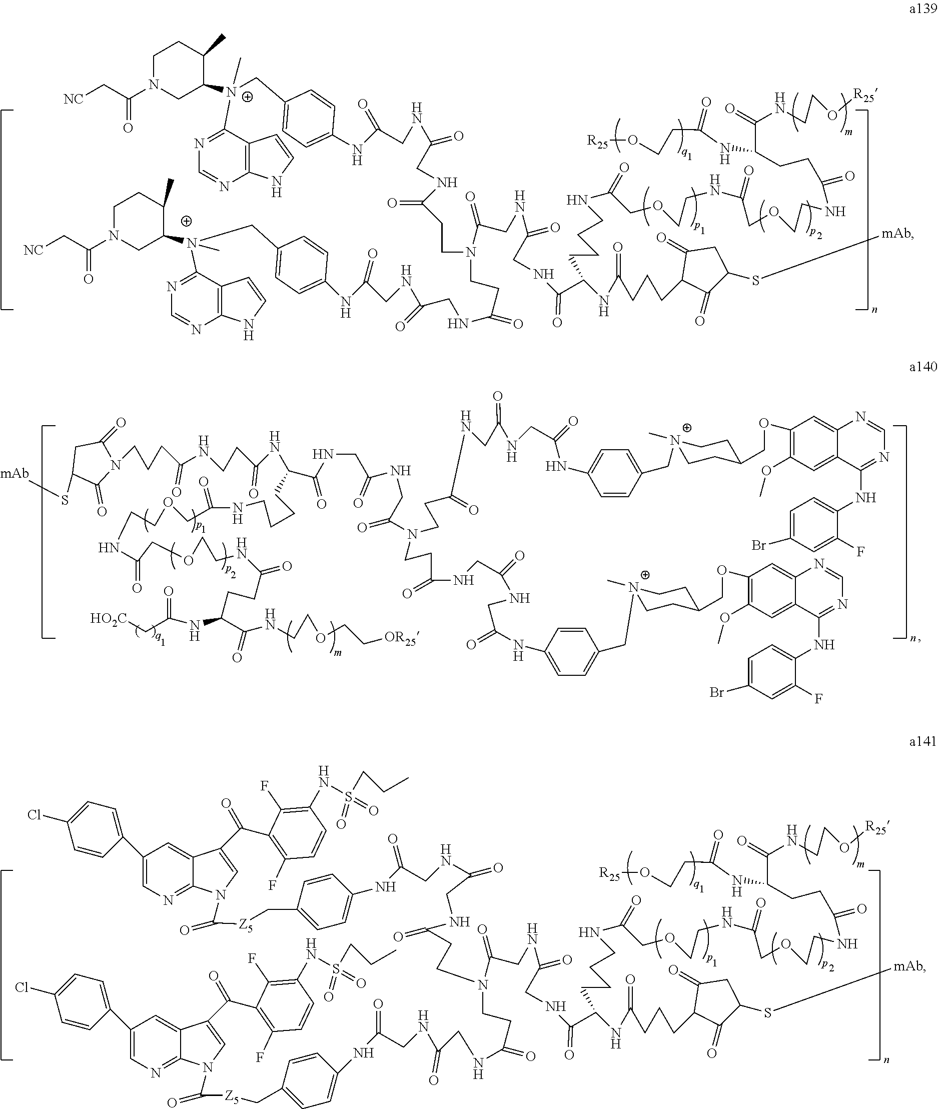

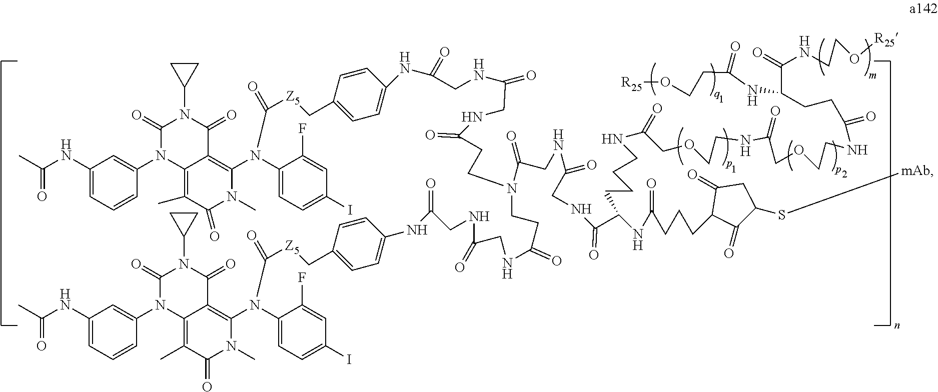

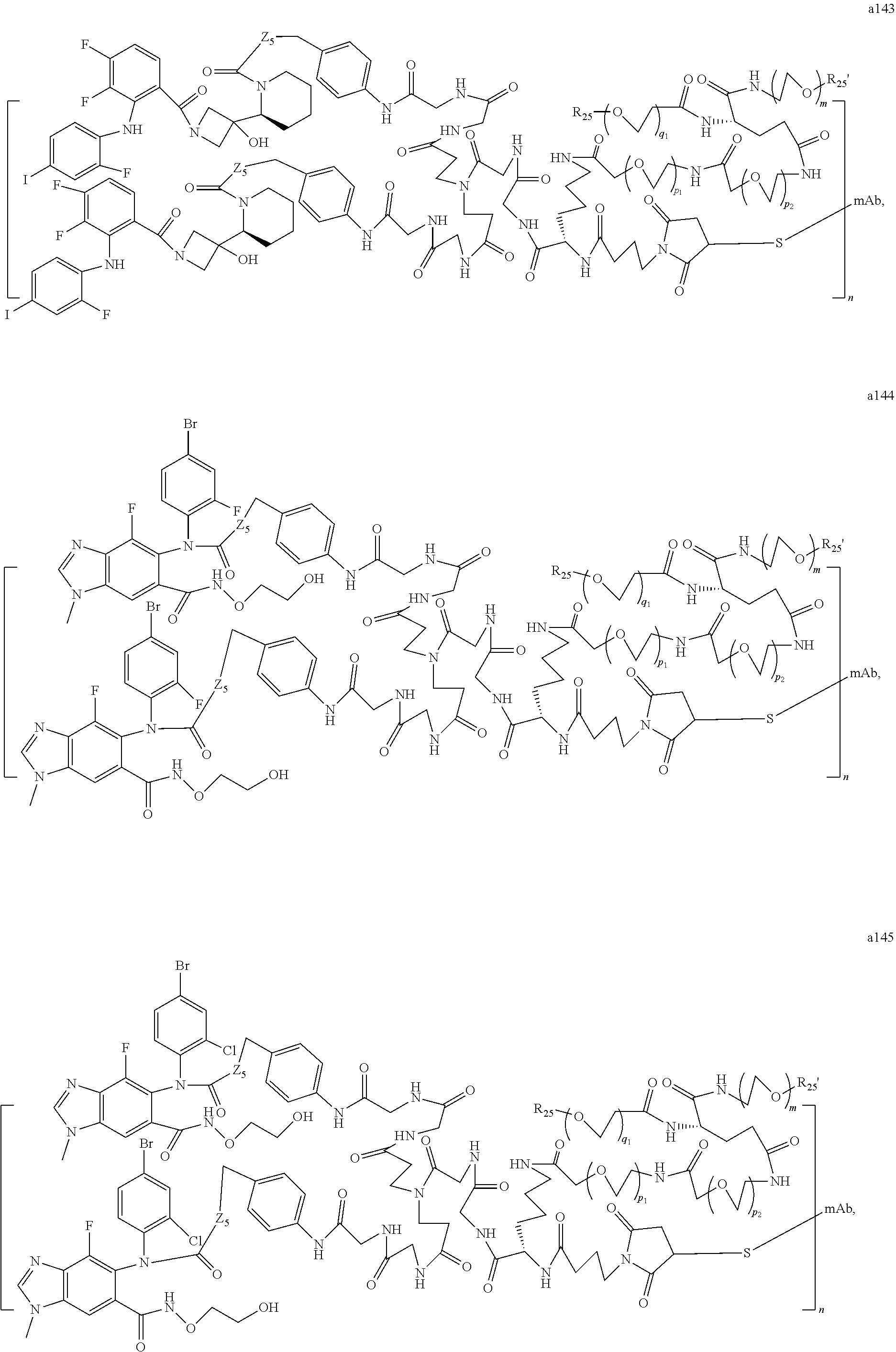

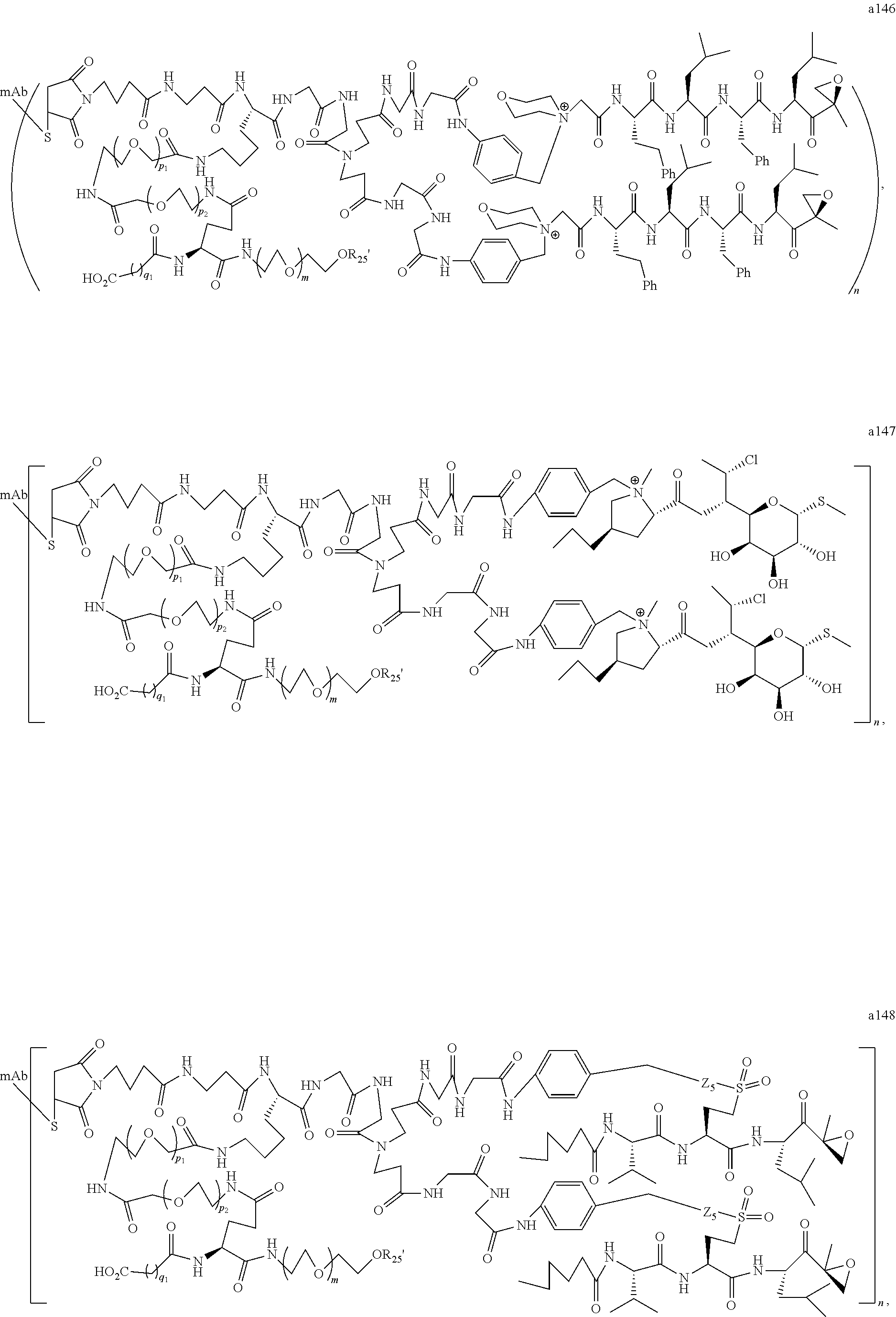

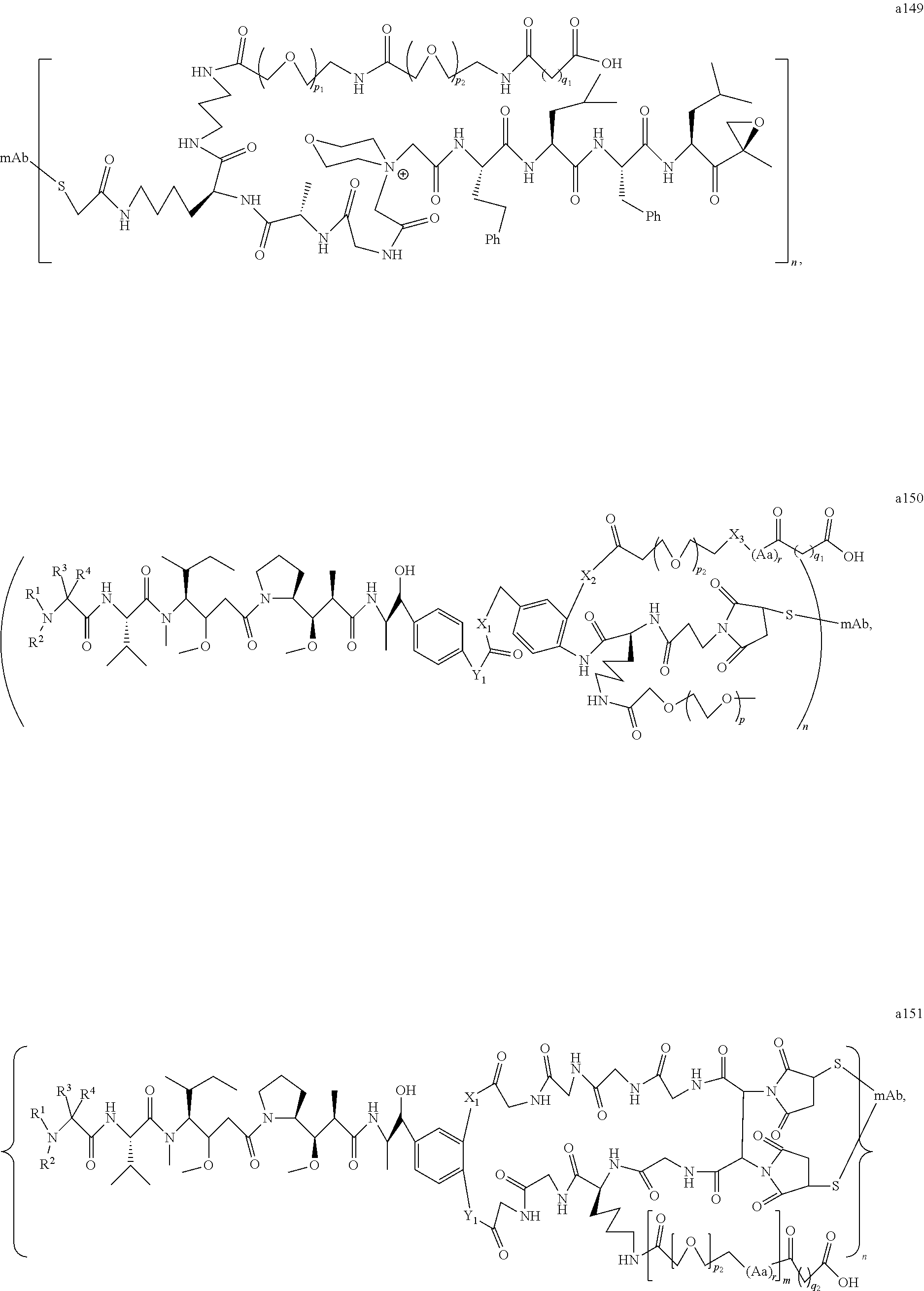

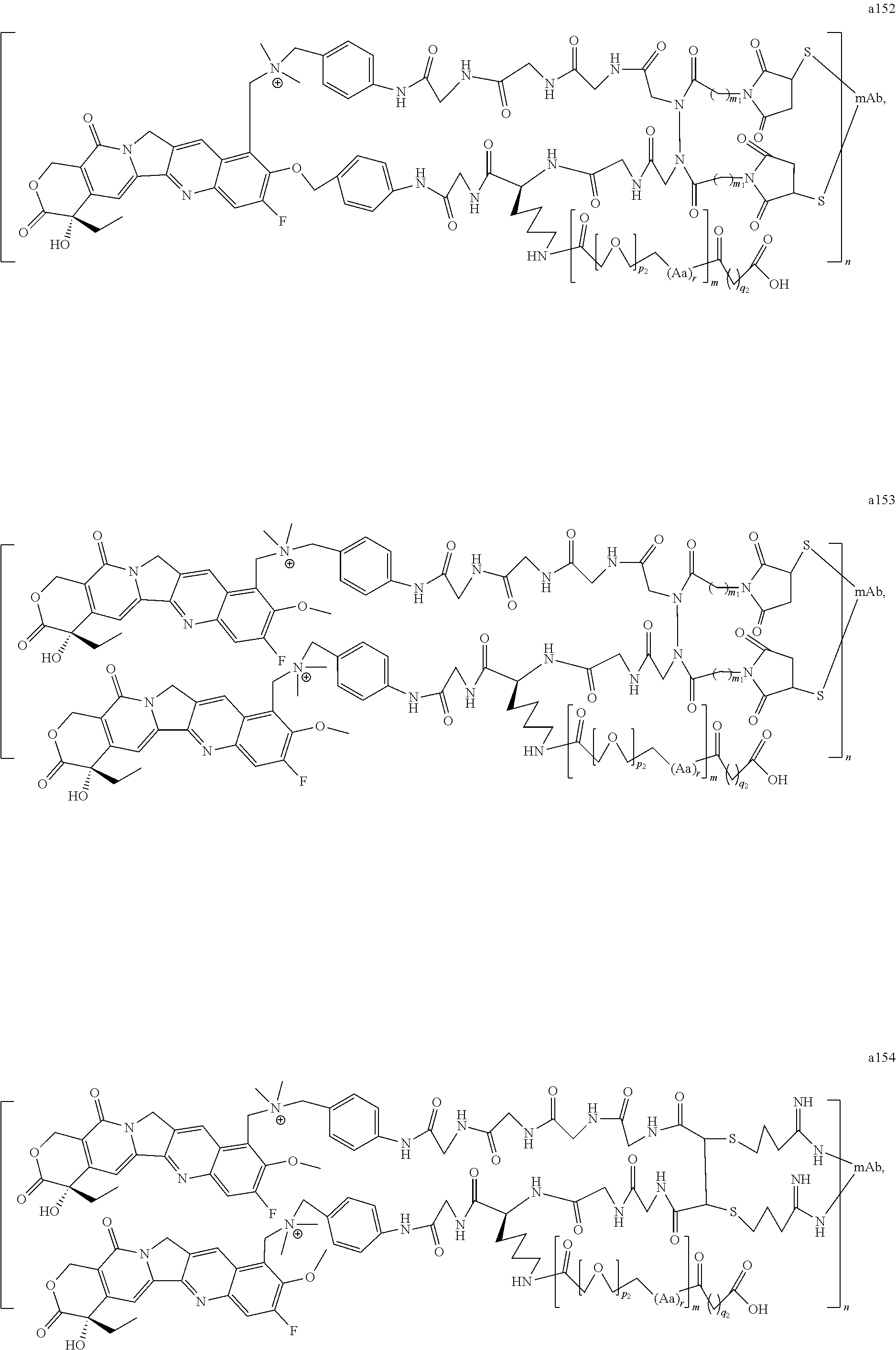

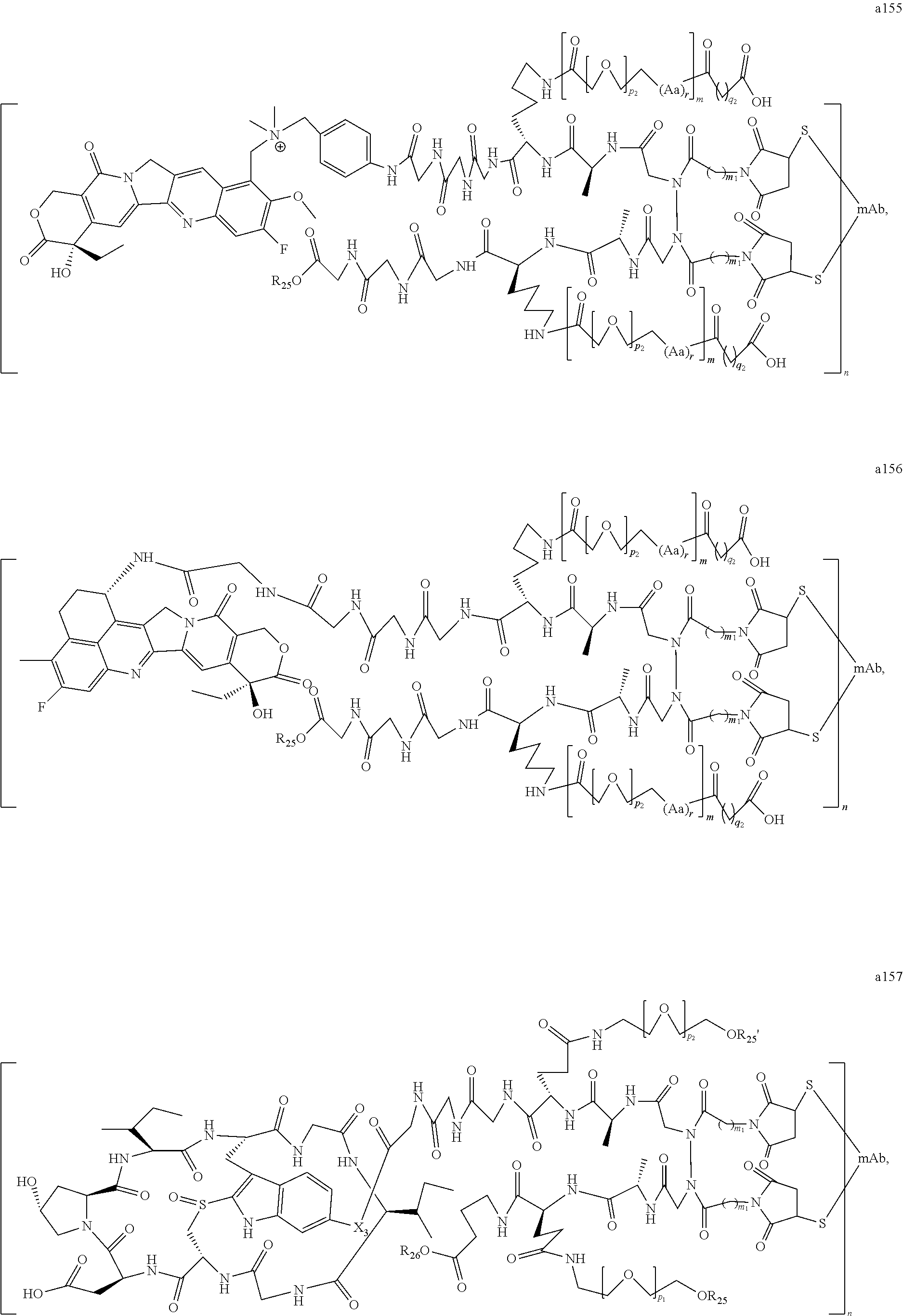

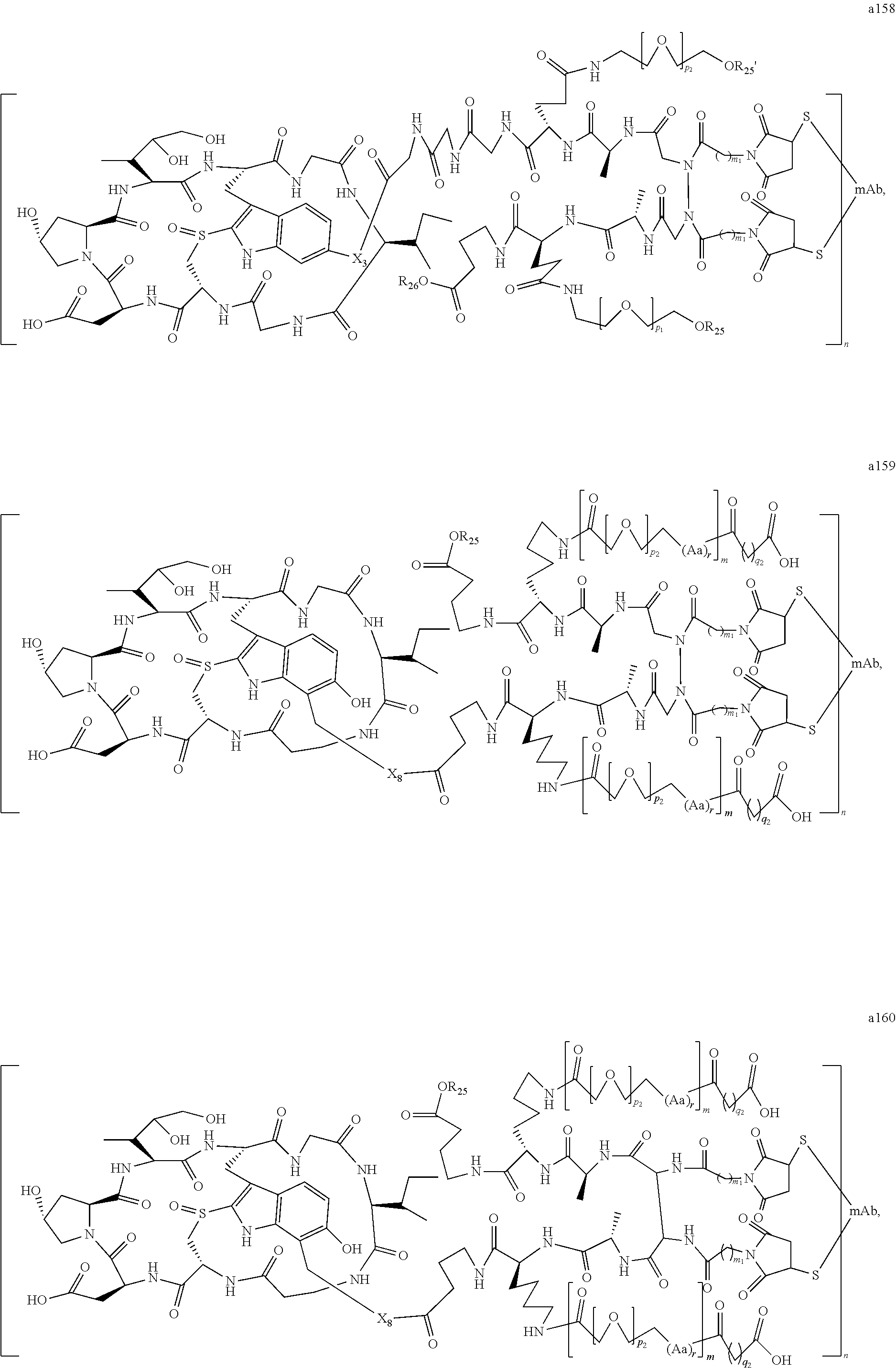

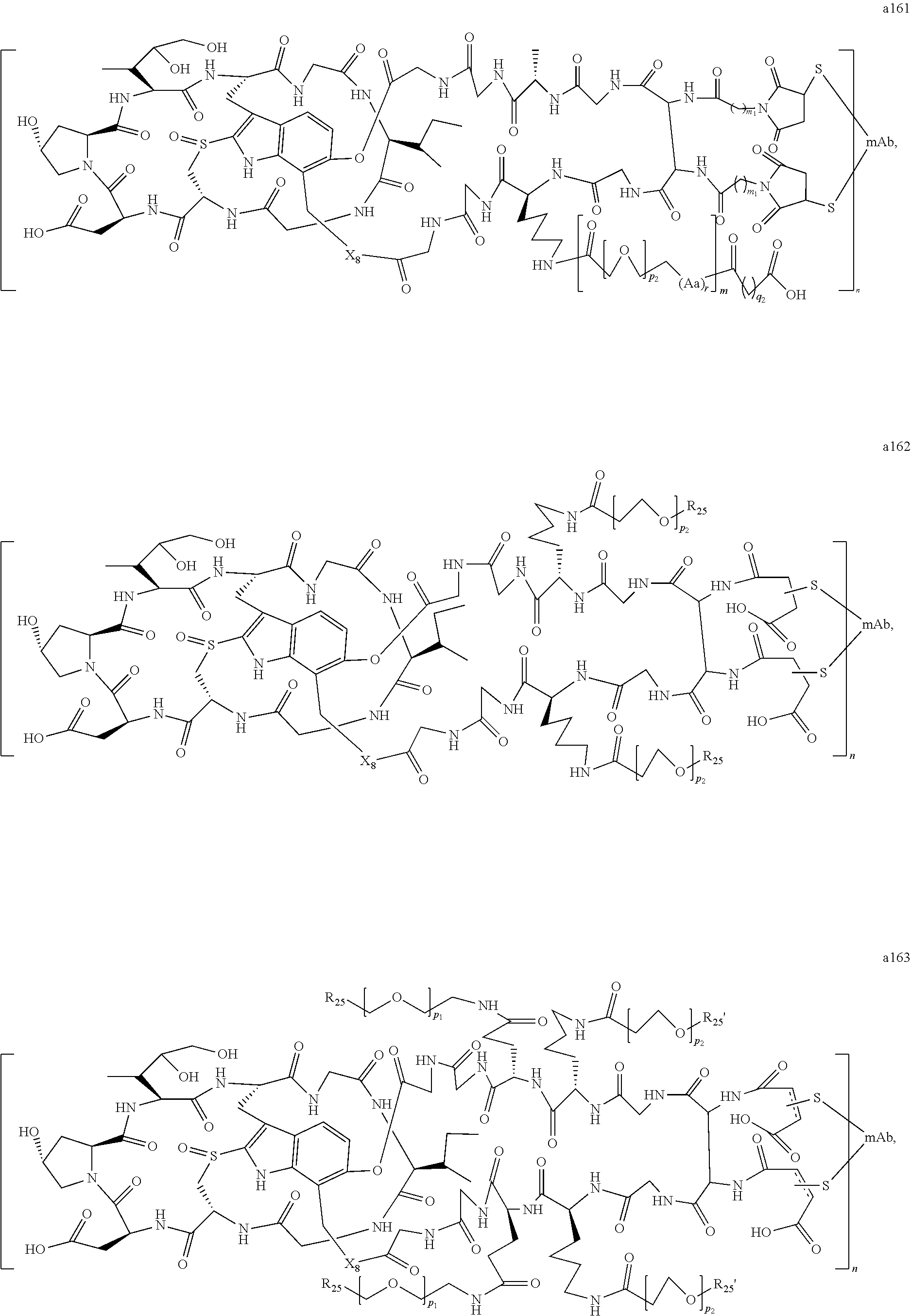

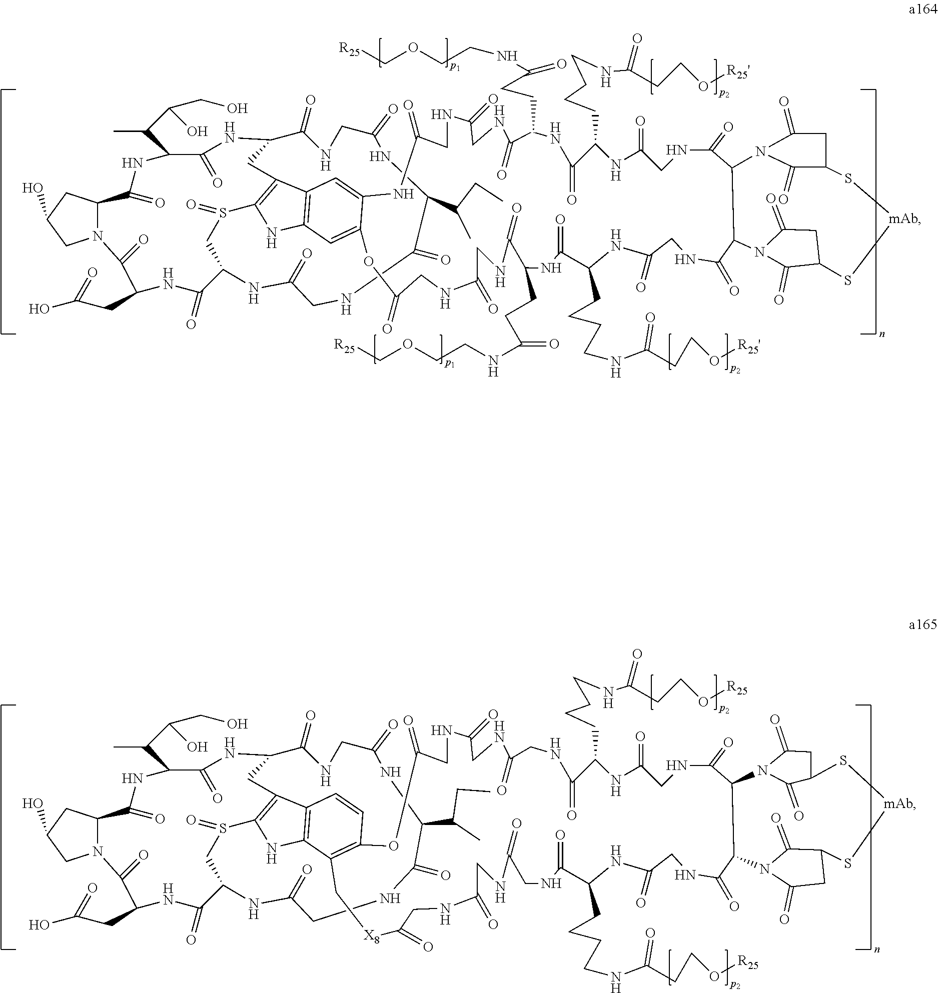

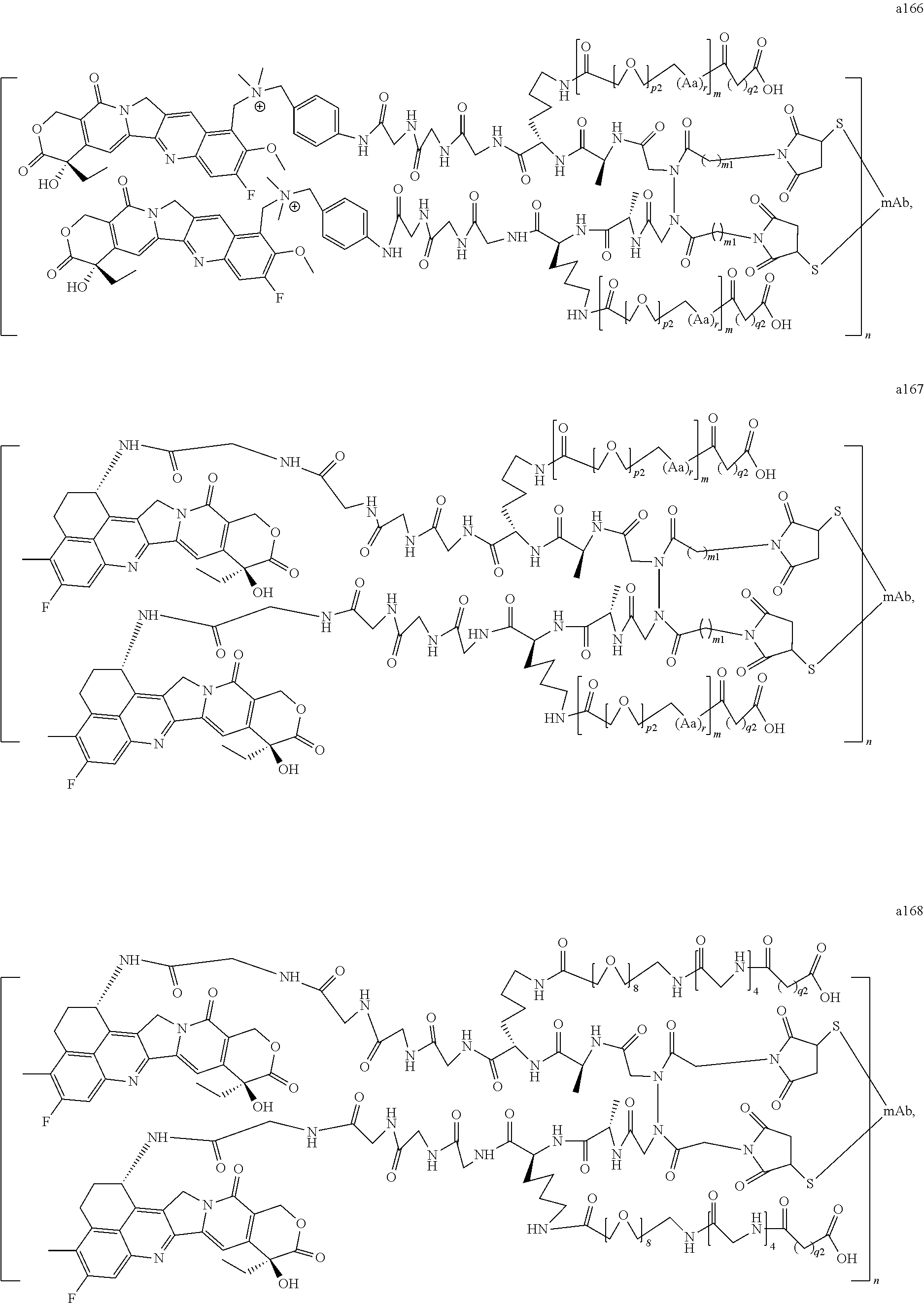

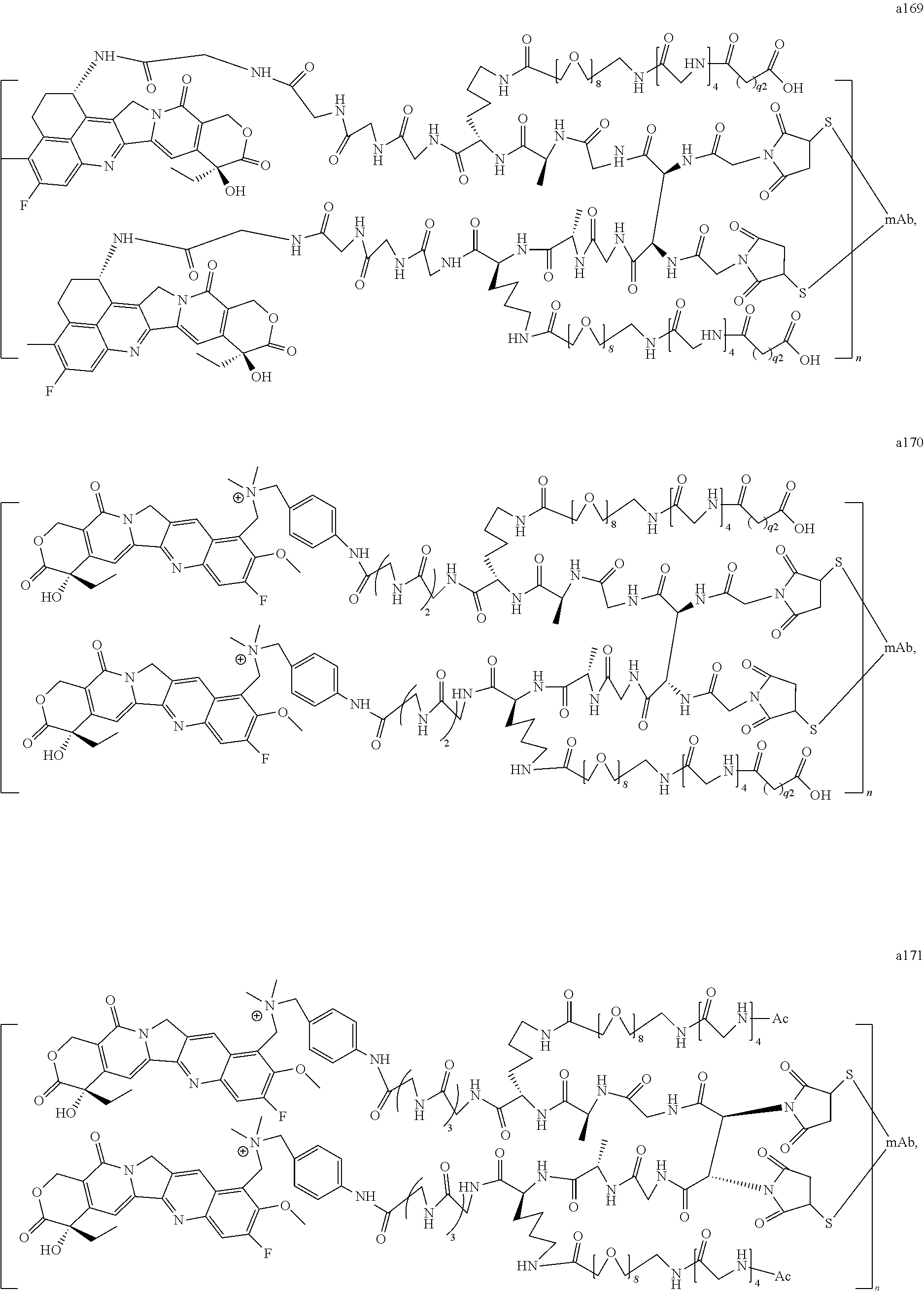

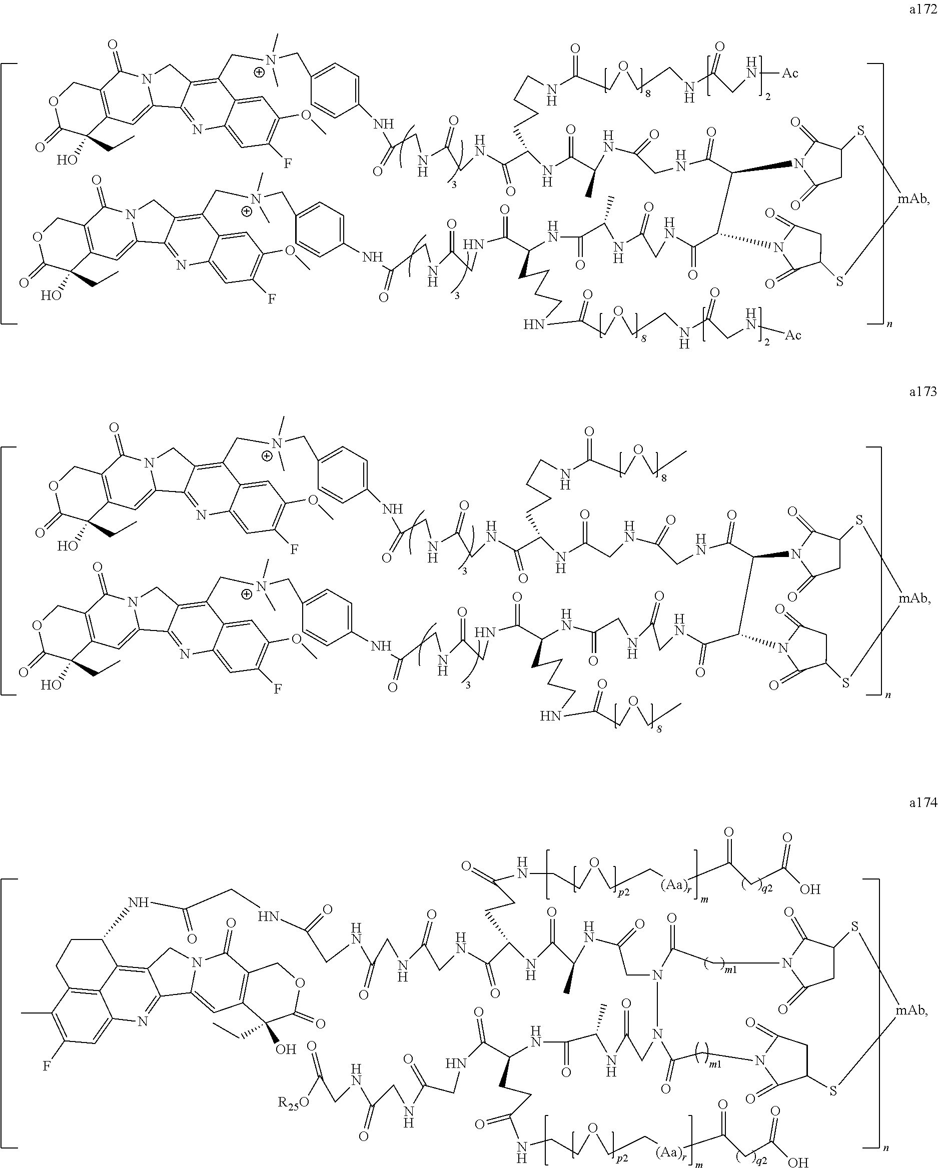

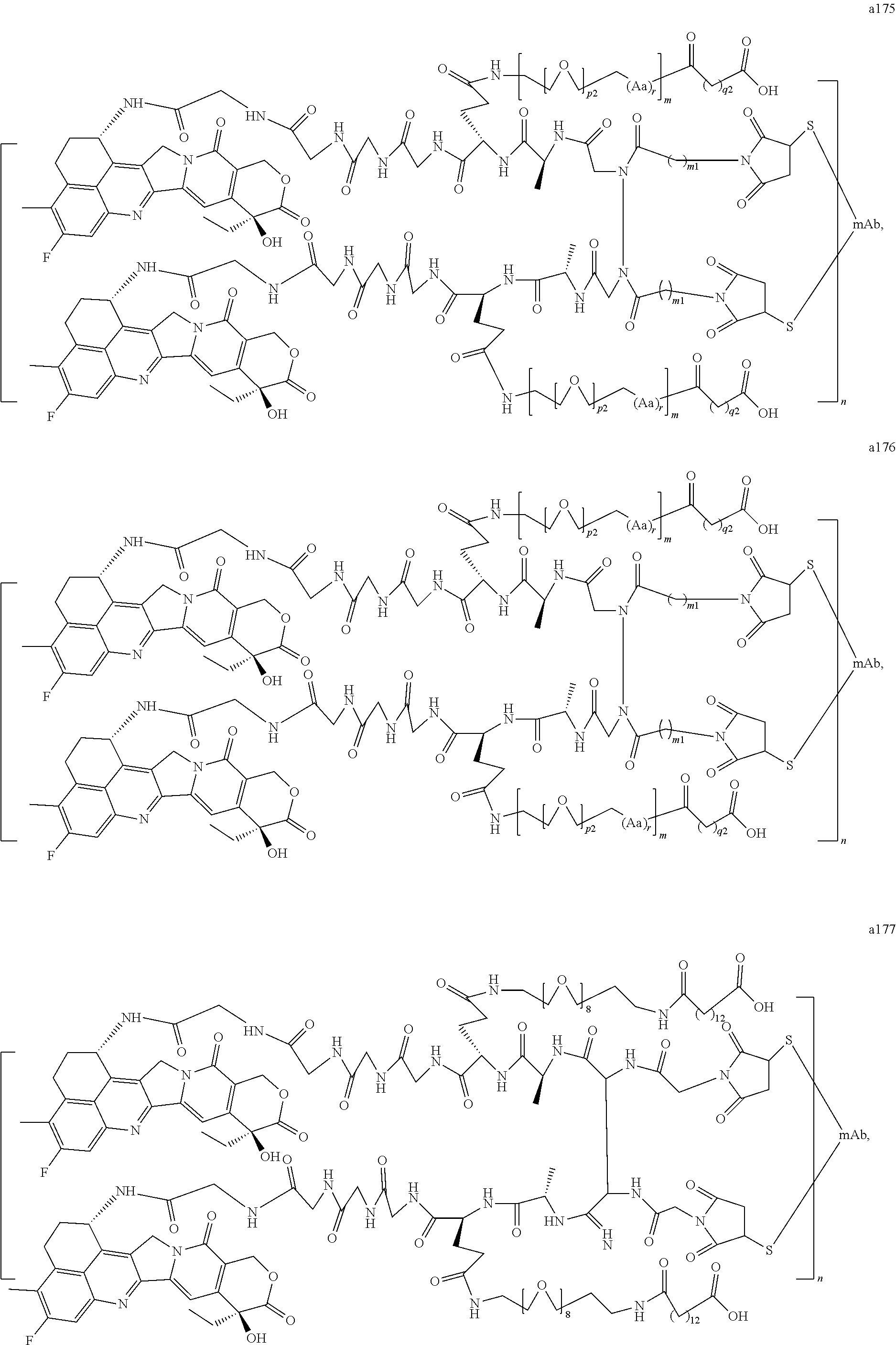

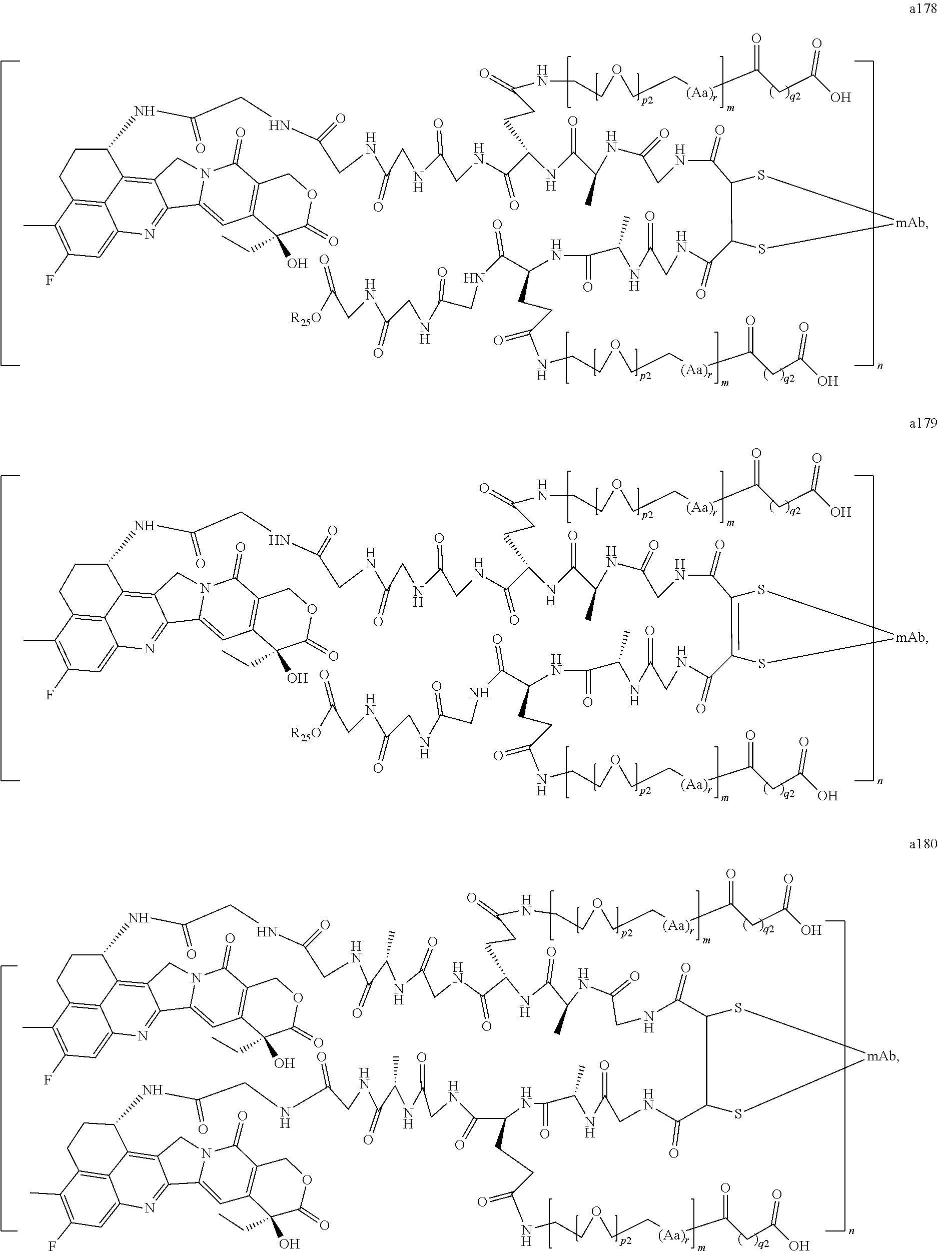

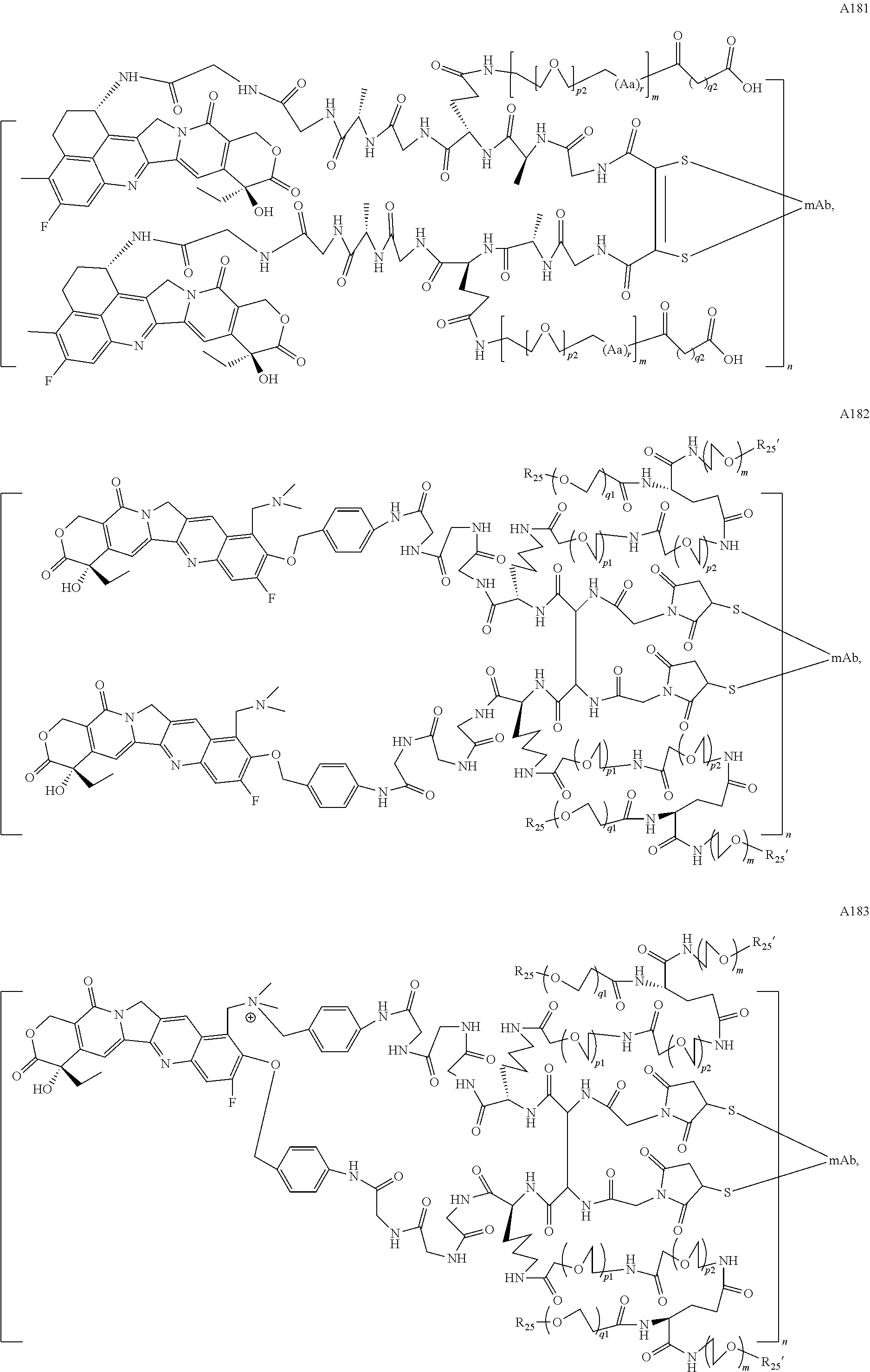

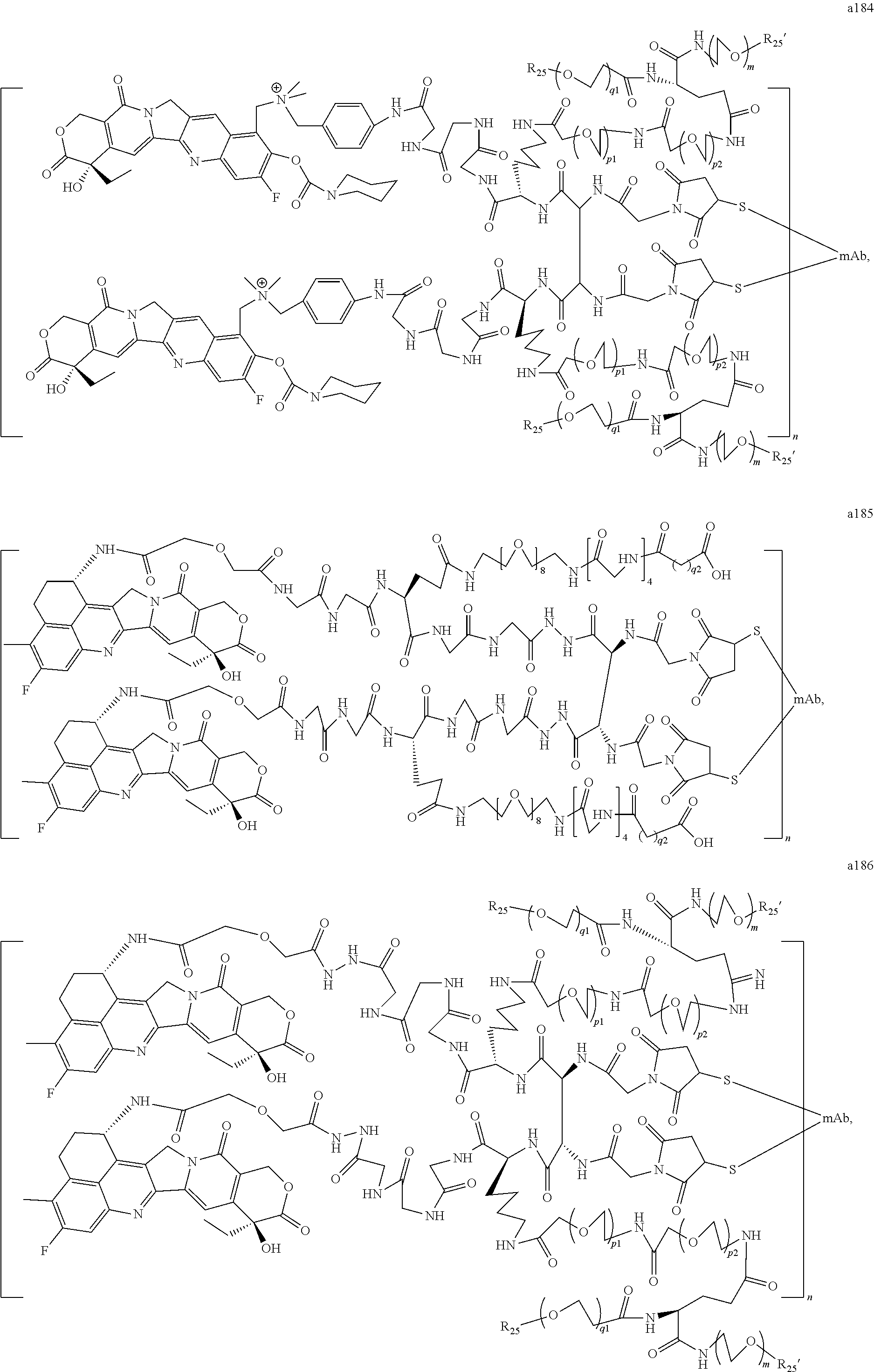

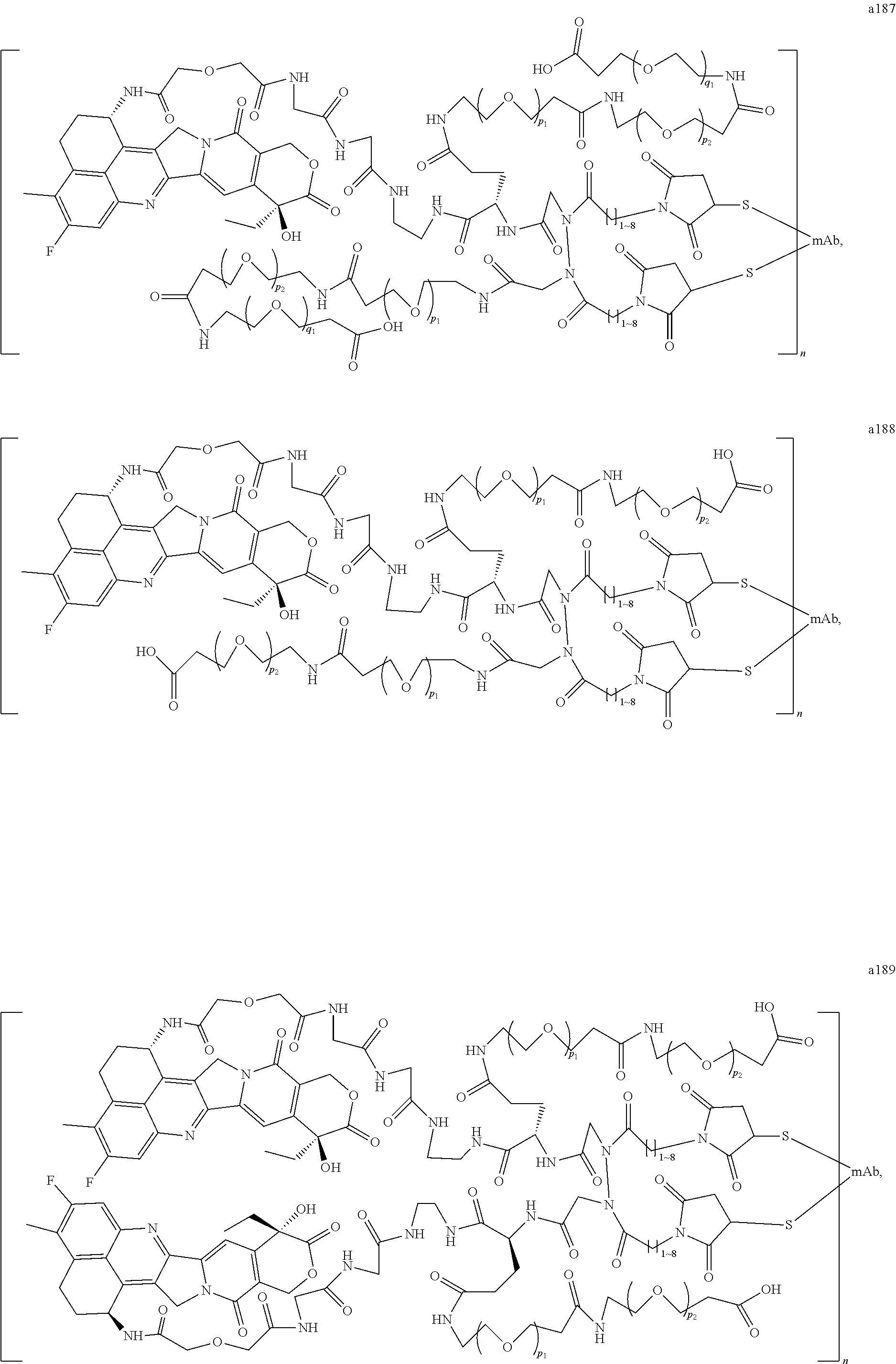

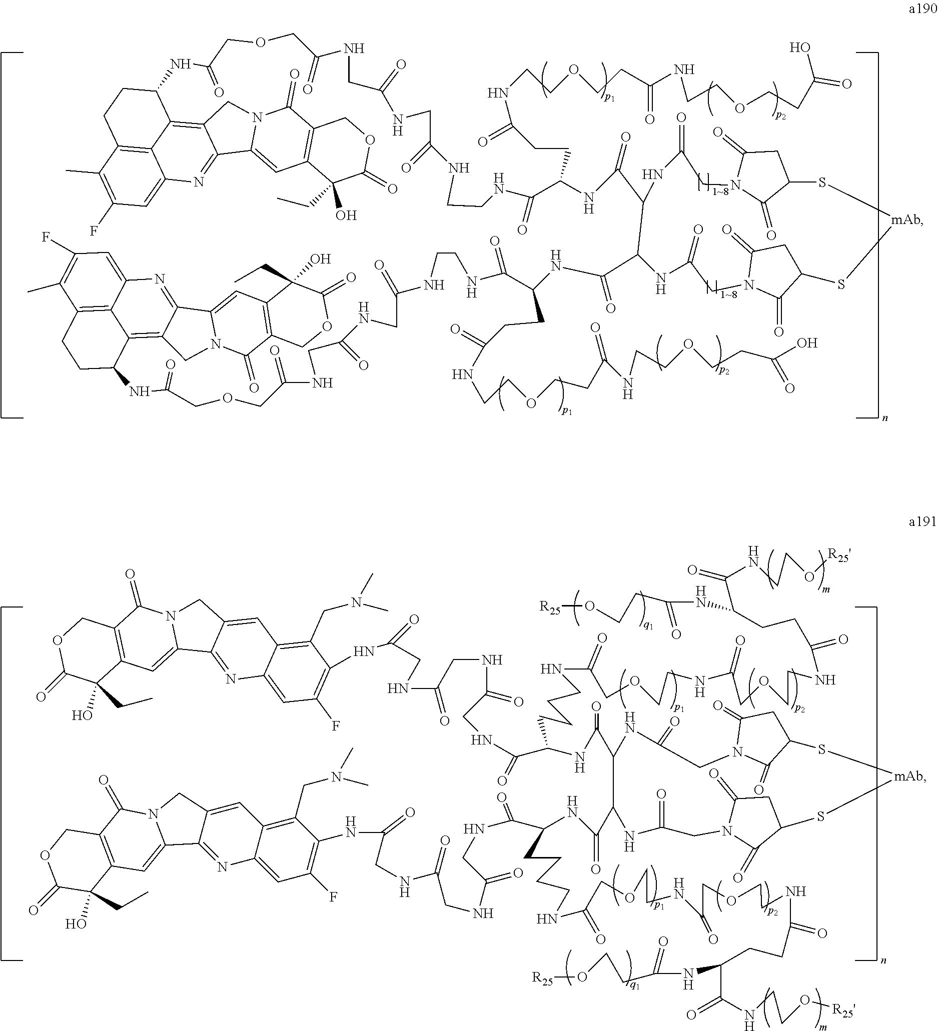

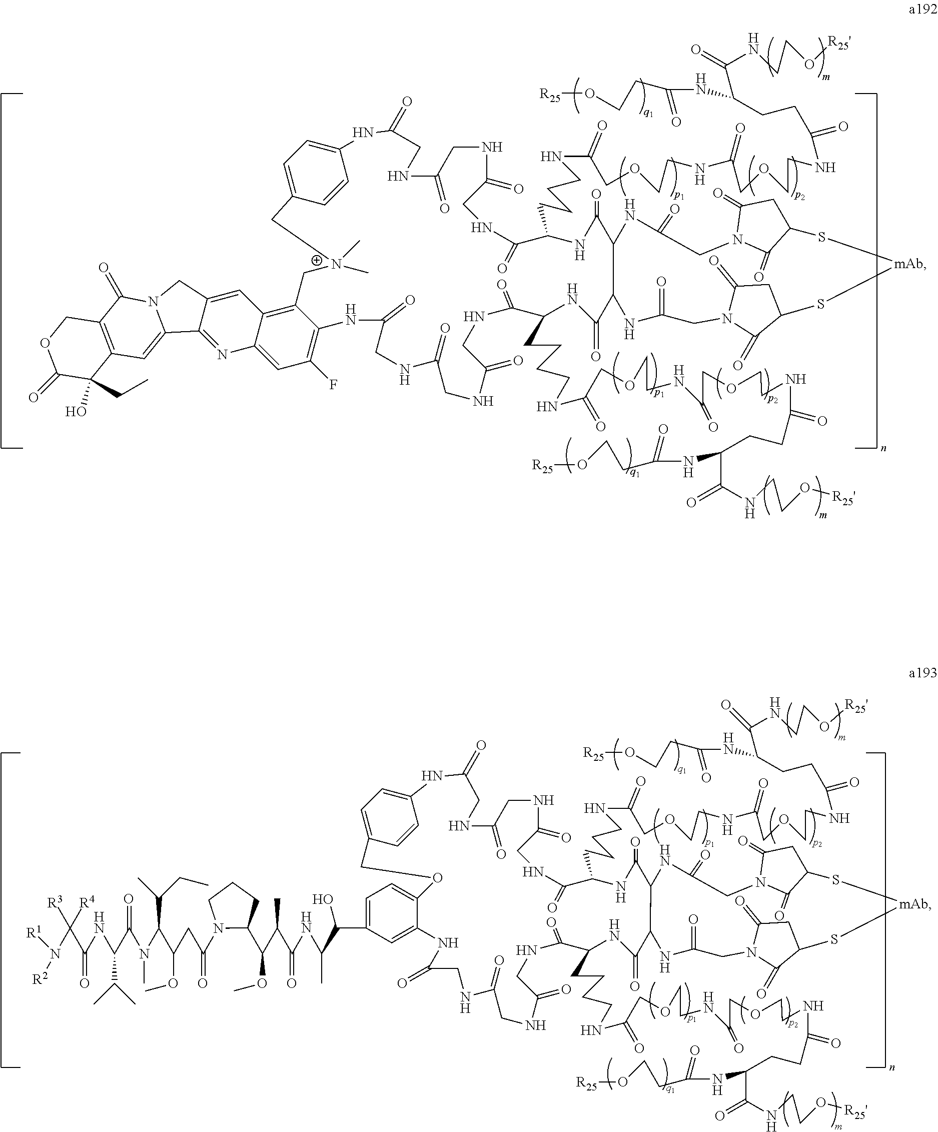

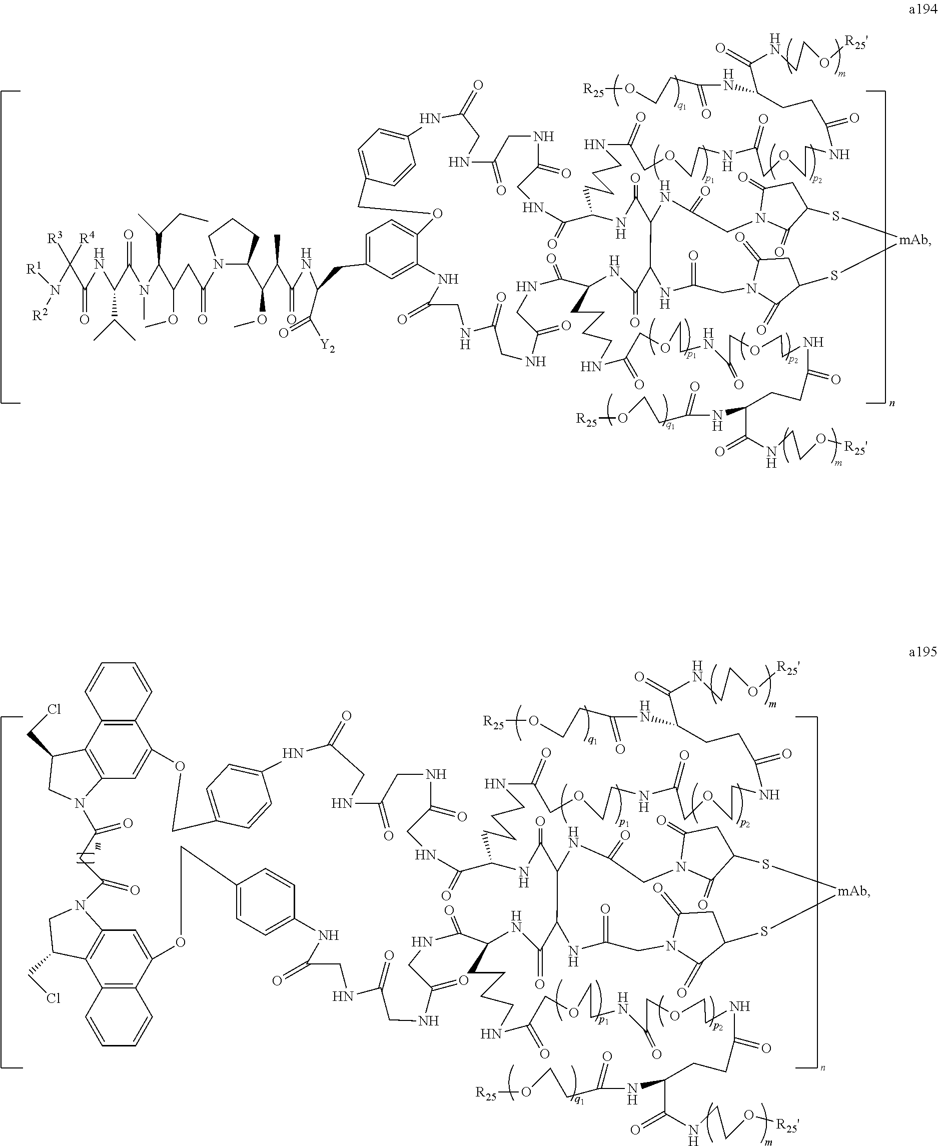

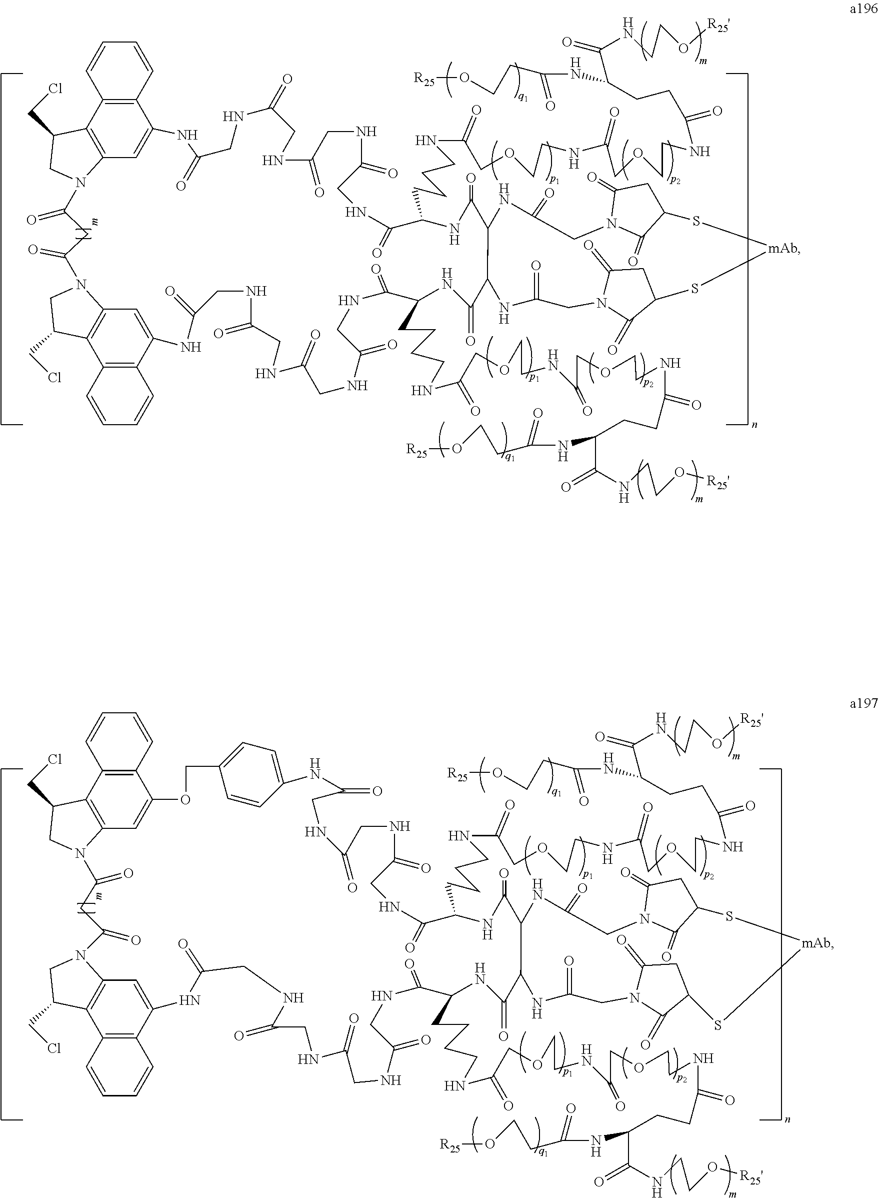

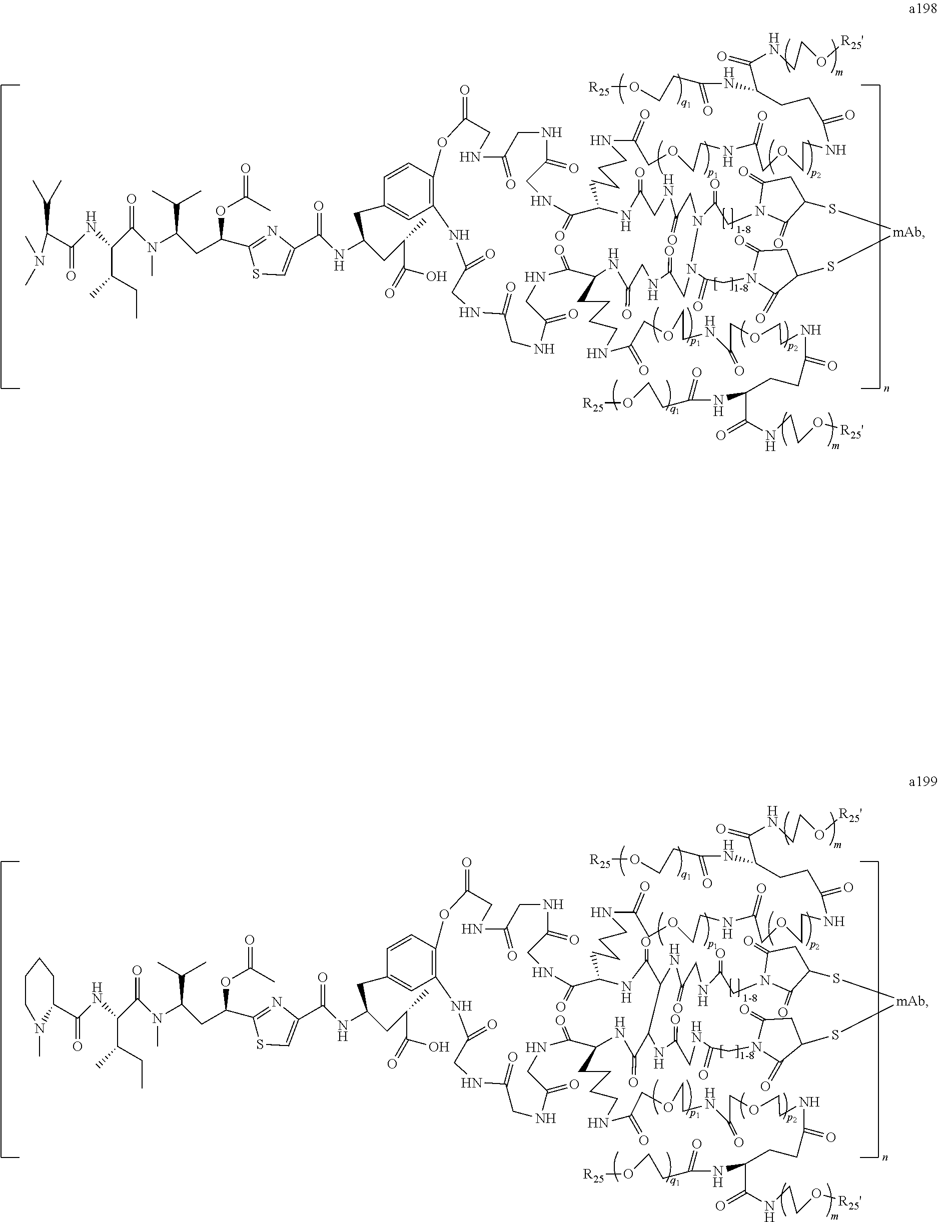

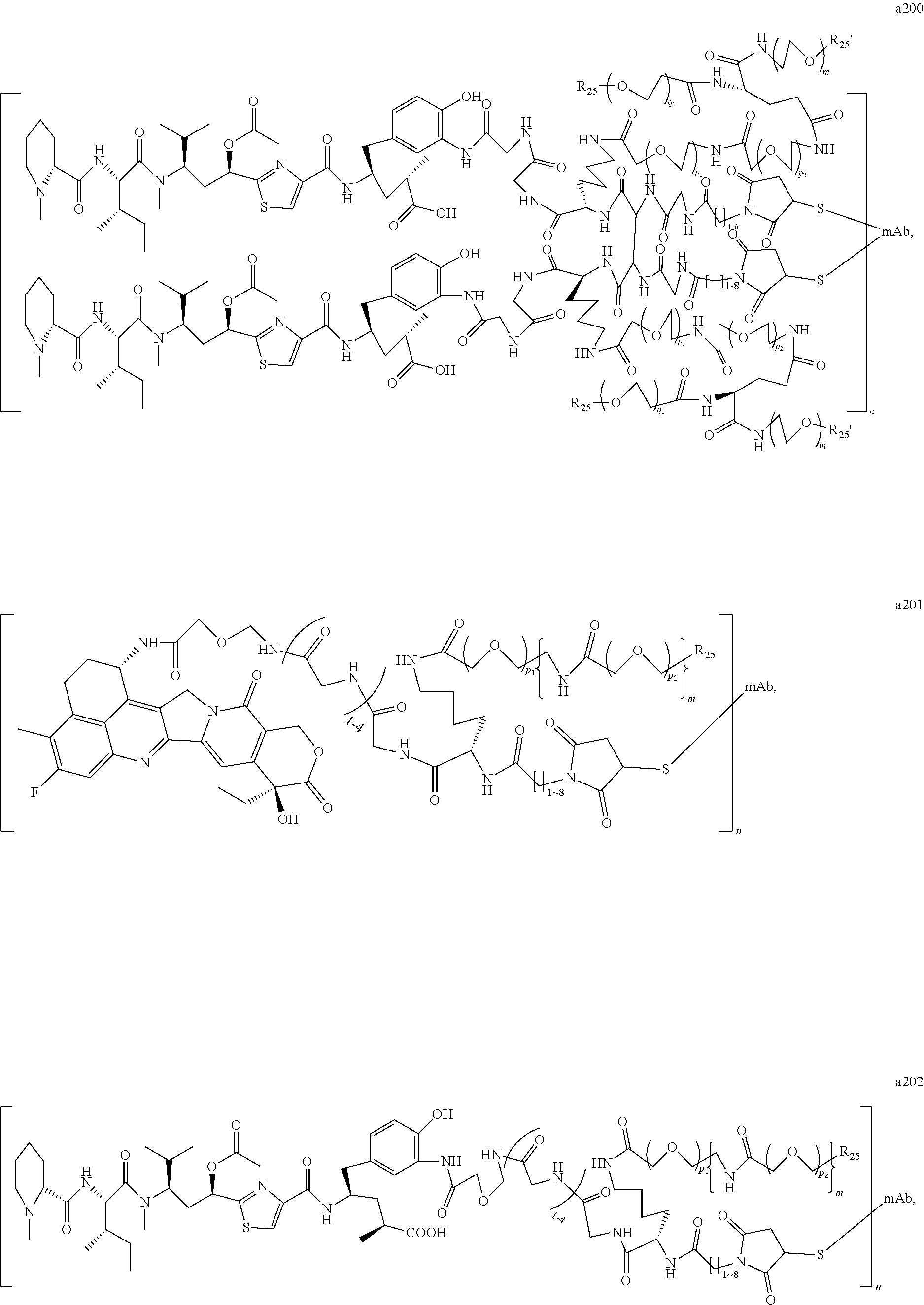

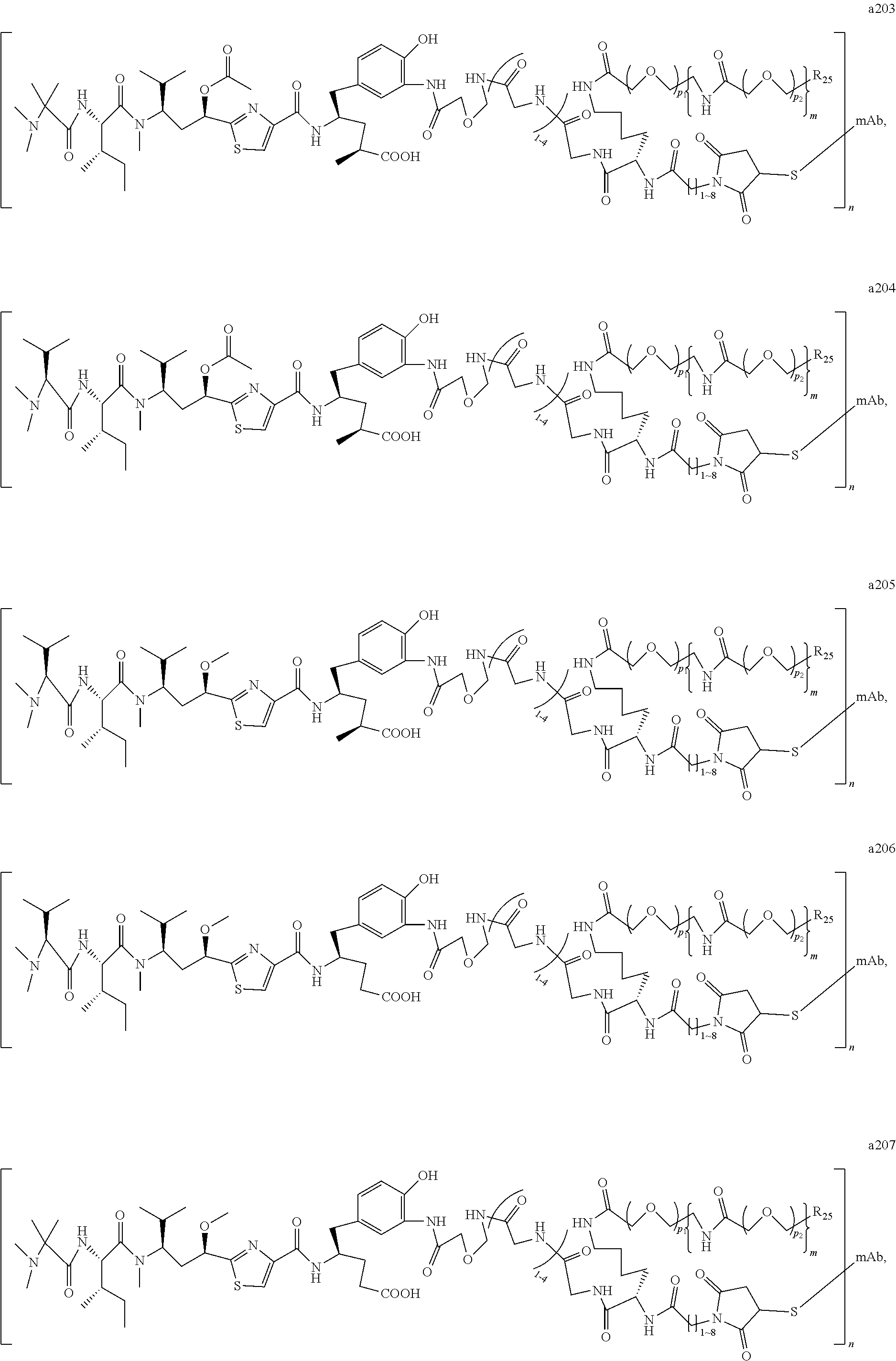

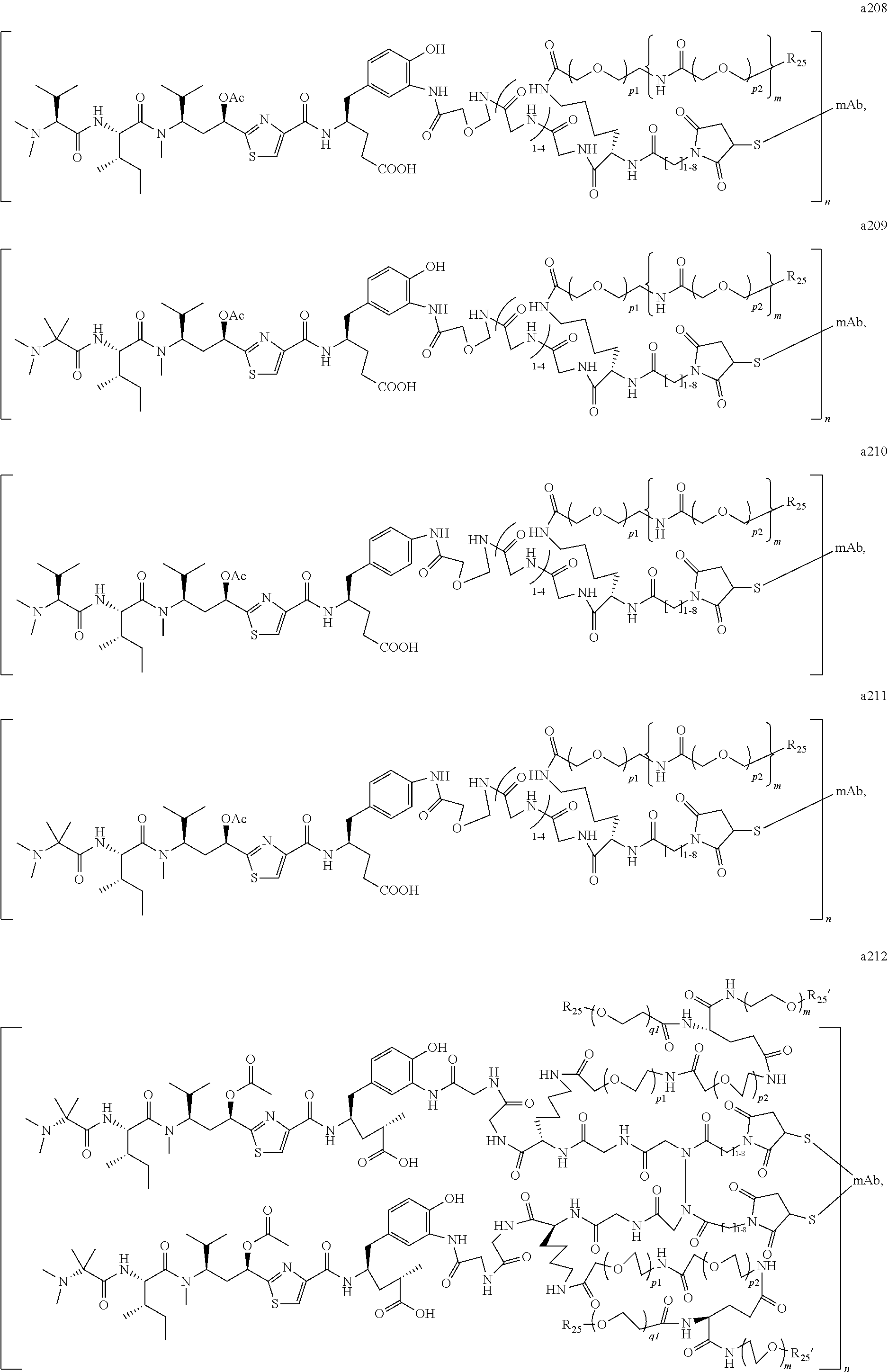

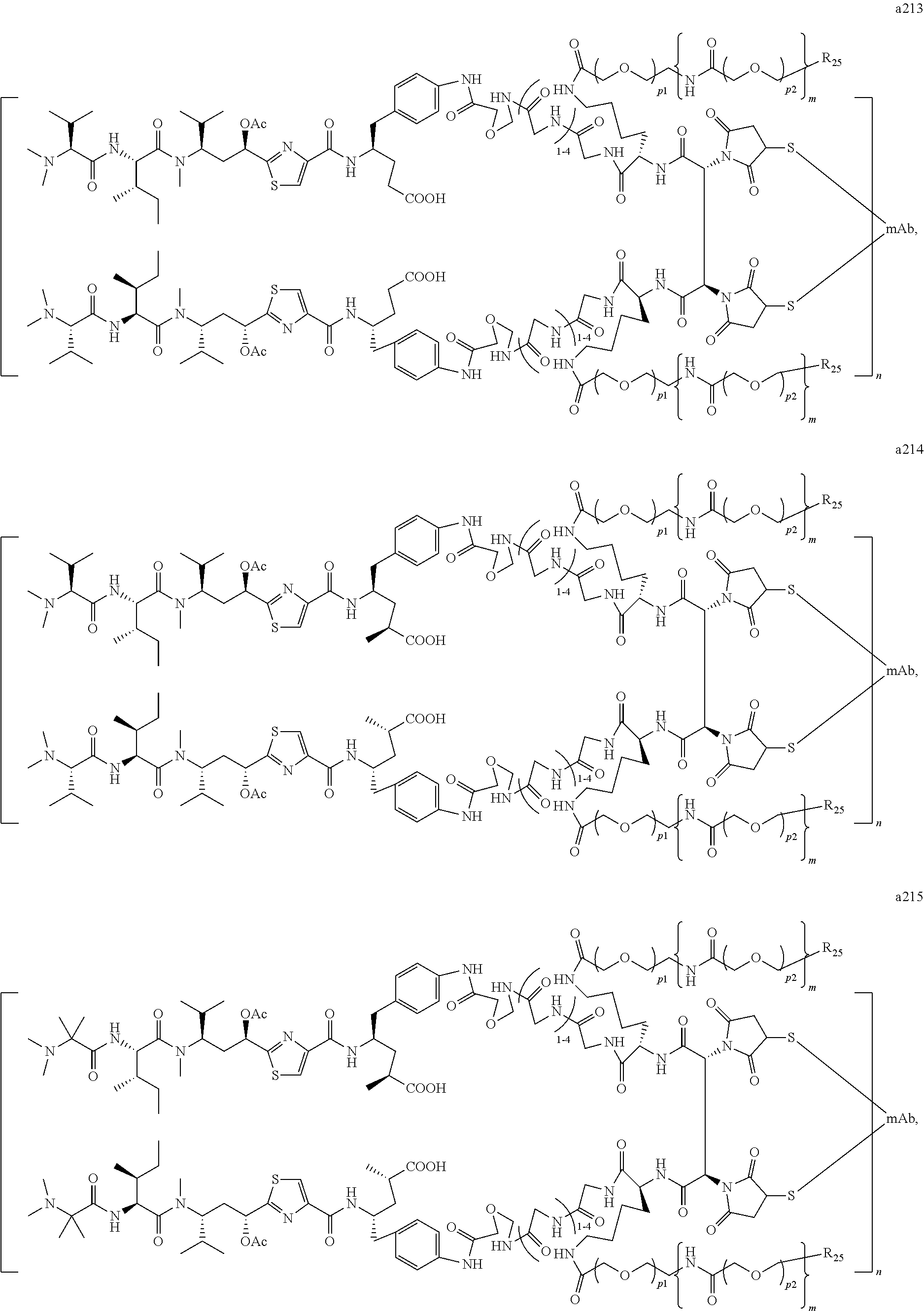

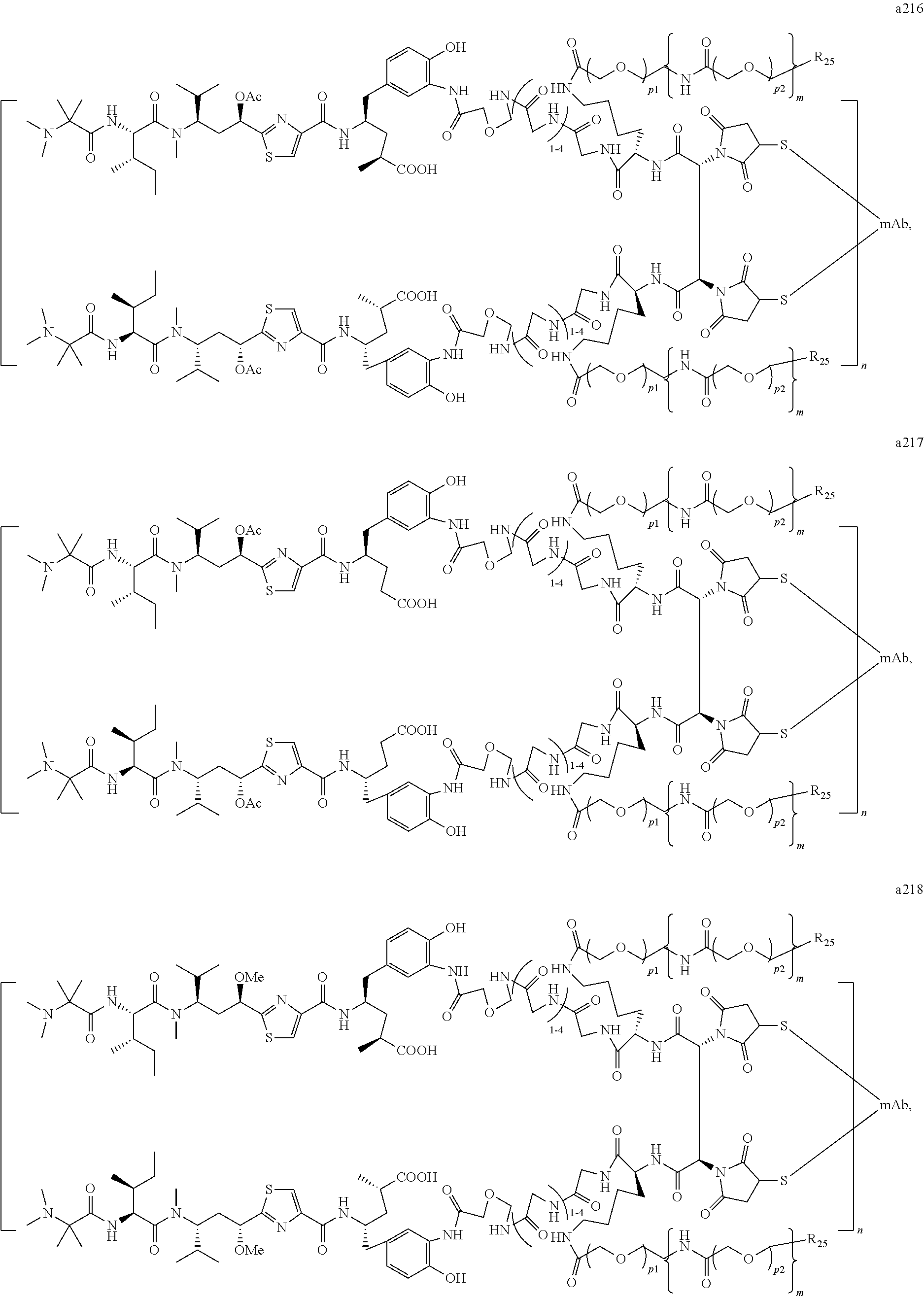

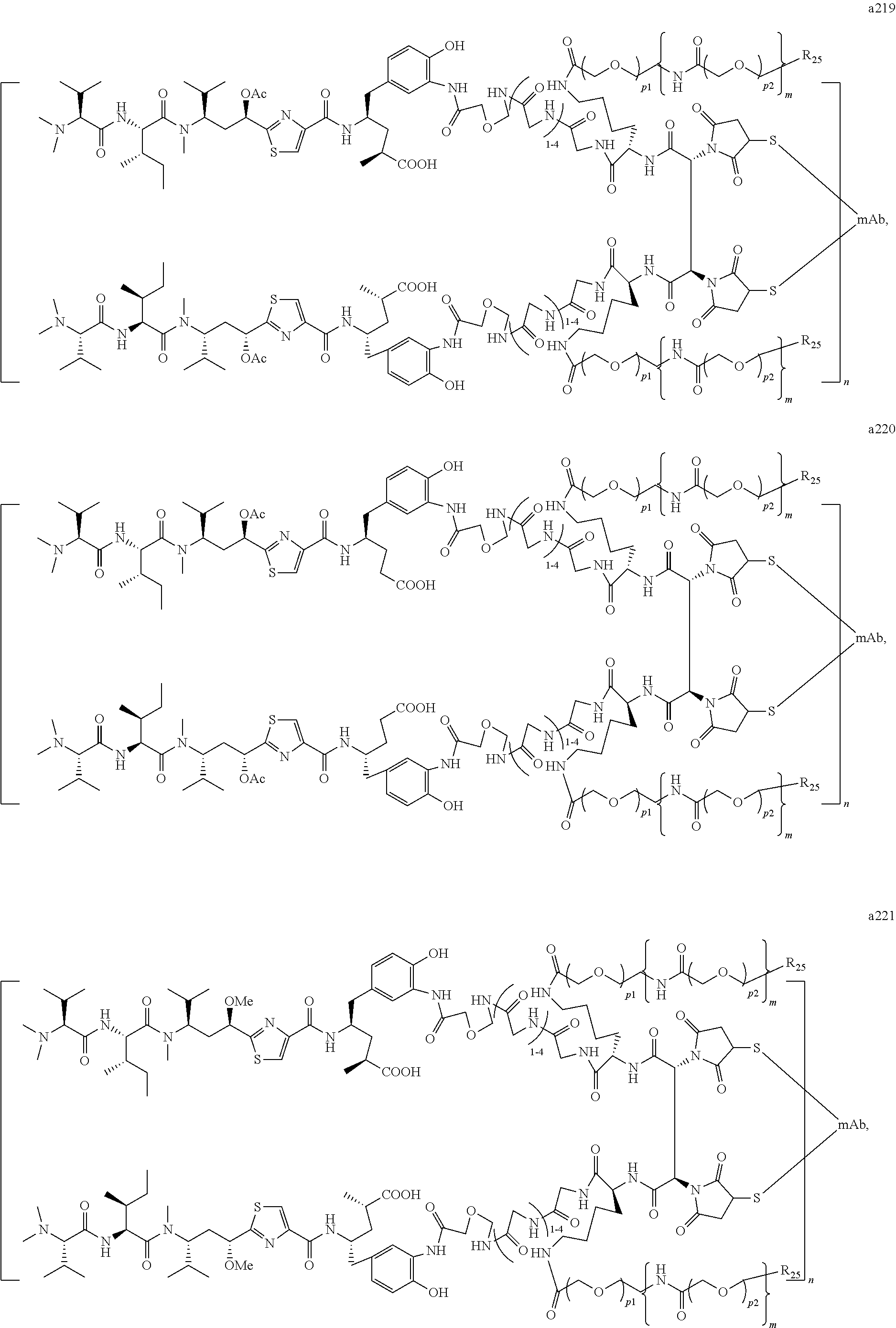

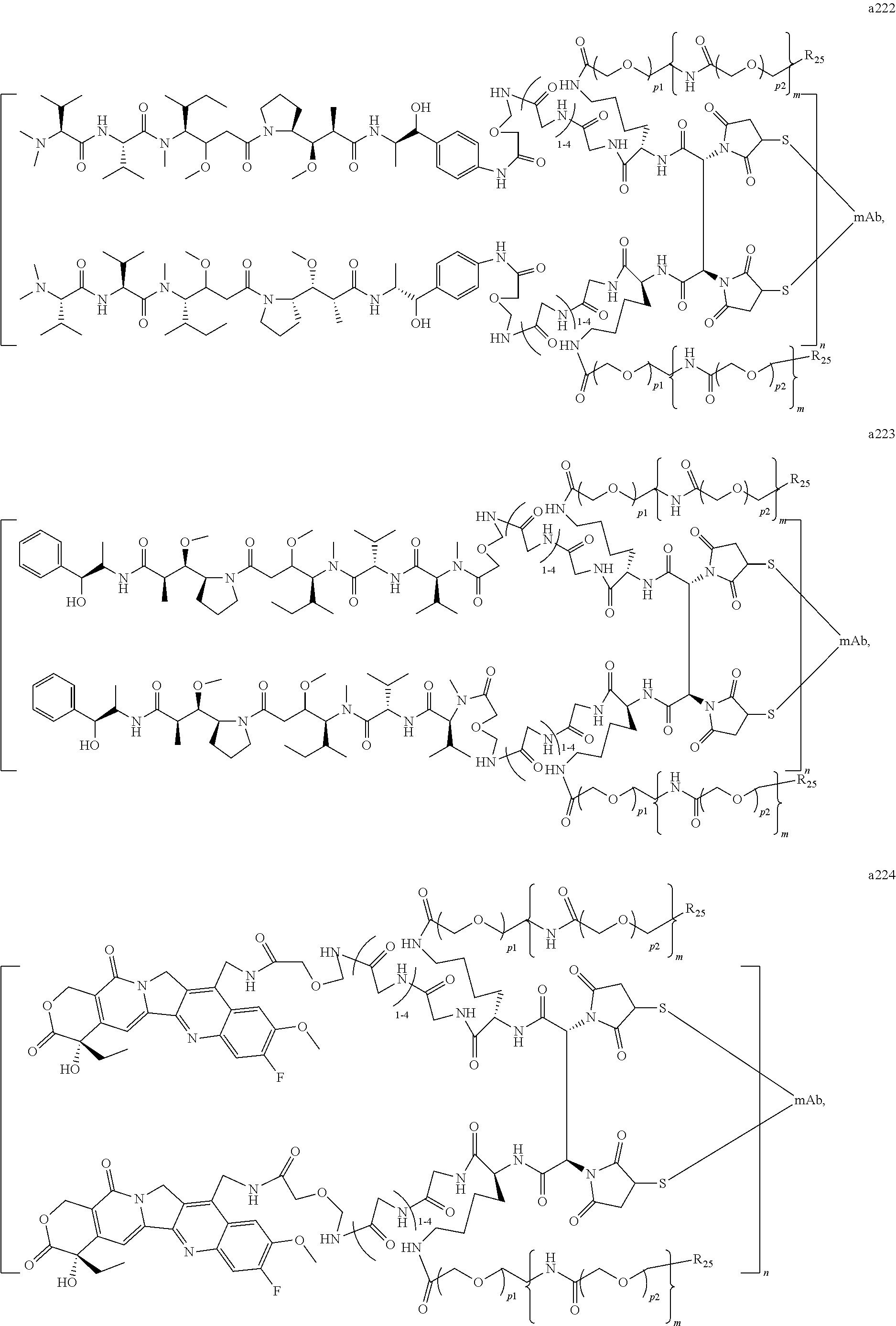

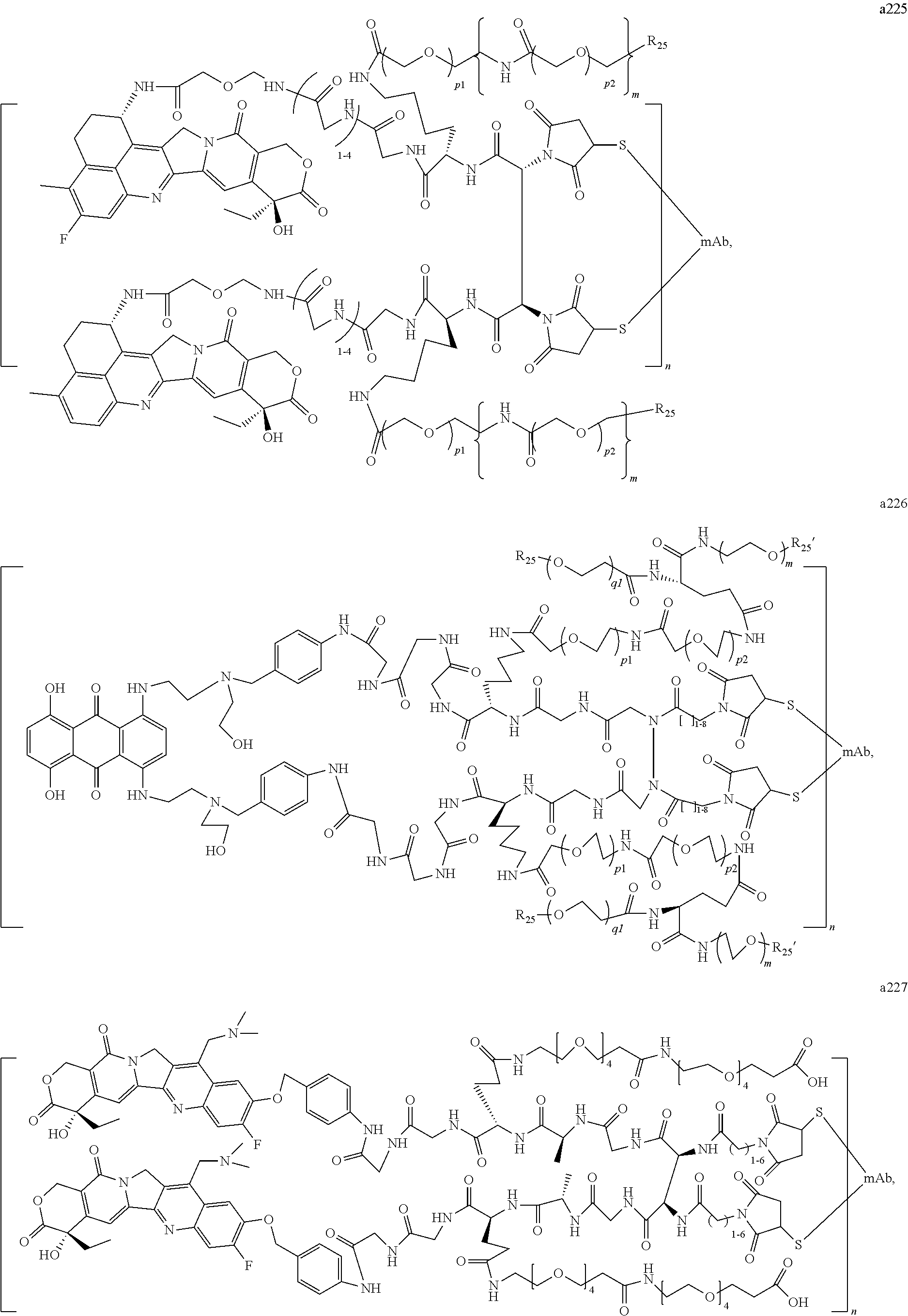

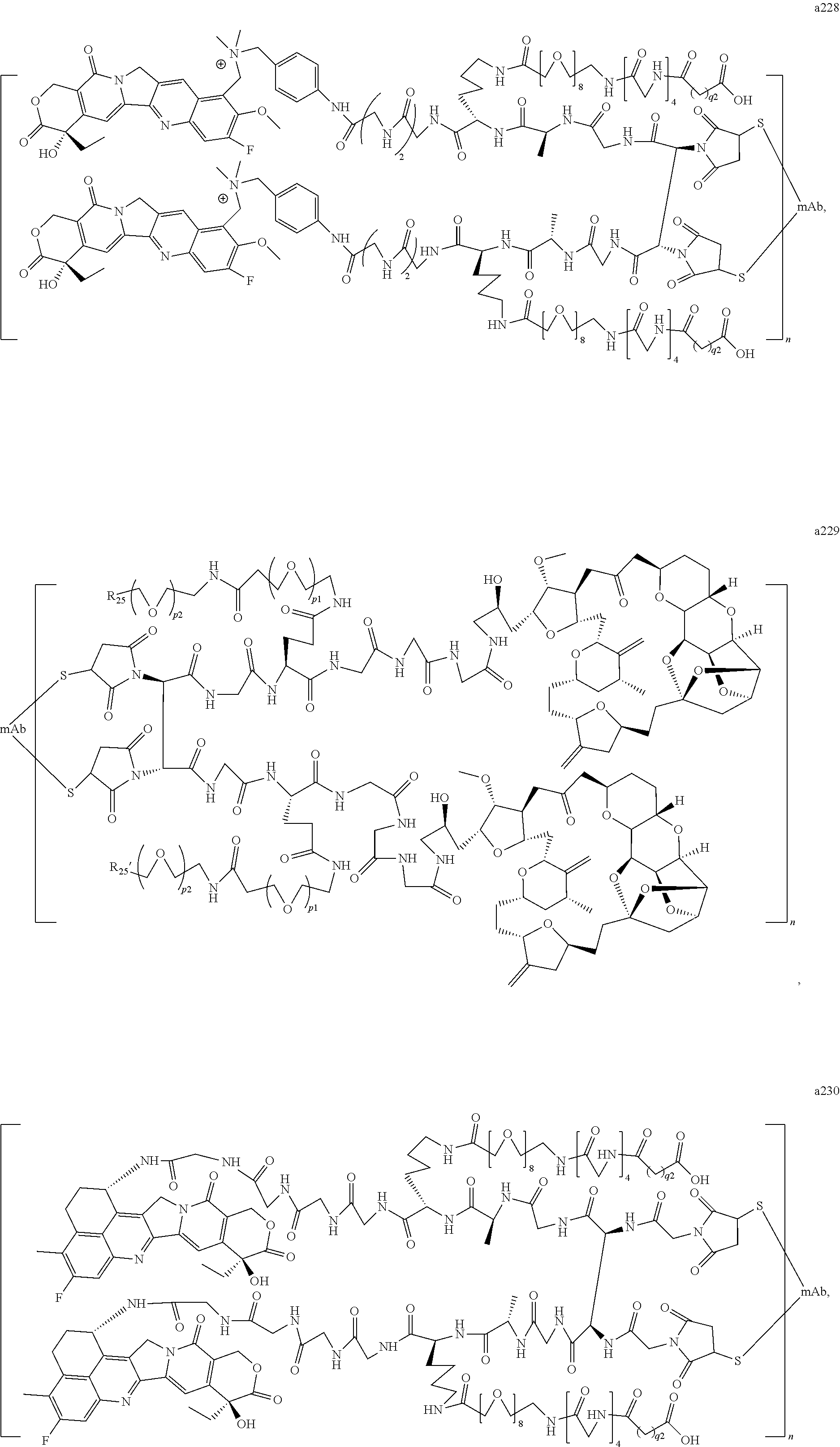

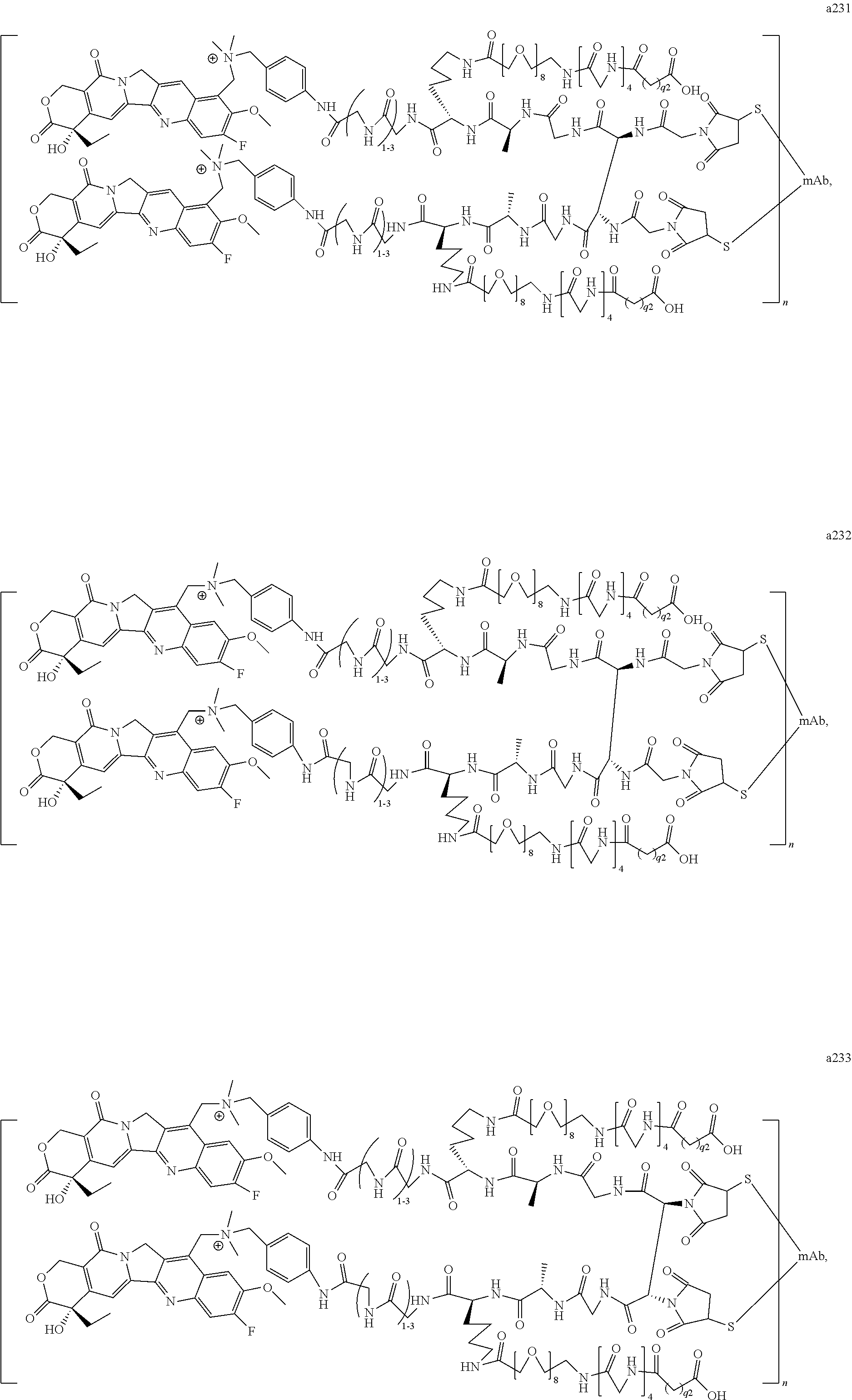

or one or more isotope of chemical elements, pharmaceutically acceptable salts, hydrates, or hydrated salts; or the polymorphic crystalline structures of these compounds; or the optical isomers, racemates, diastereomers or enantiomers;
wherein p. p₁, p₂, and p₃ are independently 0-100; m, m₁, and m₂ are independently 0-20; n is 1-10;

P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO(OR¹⁸), O—(C₄-C₁₂-glycoside), OC(O)N(C₂H₄)₂CH₂N(C₂H₄)₂CH₃, O—(C₁-C₈ of linear or branched alkyl), C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸);
R¹, R², R³, R^{1′}, R^{2′}, R^{3′}, and R⁴ are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹, R², R¹R³, R²R³, R³R⁴, R^{1′}R^{2′}, R^{1′}R^{3′} or R^{2′}R^{3′} form a 3˜7 membered carbocyclic, cycloalkyl, heterocyclic, heterocycloalkyl, aromatic or heteroaromatic ring system; R₄, R₅, R₅′, and R₆, are independently H, C₁-C₈ linear or branched alkyl, C₃-C₈ aryl, heteroaryl, heteroalkyl, alkylcycloalkyl, acyloxylamines, or(Ar)ᵣ, r=1-6 (amino acid or peptides having the same or different sequence of amino acids);
R₇, R₈, and R₉ are independently H, OH, OR₁, NH₂, NHR₁, C₁-C₆ alkyl, or absent;
R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent;
R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa)ᵣ is 1-8 aminoacids; n and mᵢ are independently 1-20; p is 1-1000; R₁, R₂ and Ar, are the same defined through out the application; “ [symbol] ” is defined the same above;
R₁₂ and R_{12′} are independently H, ═O, OR₁, NH₂, NH(CH₃), NHNH₂, COOH, SH, OZ₃, SZ₃, F, Cl, or C₁-C₈ linear or branched alkyl, C₃-C₈ aryl, heteroaryl, heteroalkyl, alkylcycloalkyl, acyloxylamines;
R₂₅, R₂₆ and R₂₅′ are are independently H, Ac, R₁, C(O)NHR_{I}, C(O)R₁, R₁COOH, R₁COOR₂, R₁OR₂, R₁CONHR₂, CH₂OAc, CH₂NHAc, R₁NH₂, NR₁R₂, N⁺R₁R₂R₃, CH₂CONH(CH₂)_{q1}COOH, CH2CONH(CH₂)_{q1}COOR₁, CH₂CONH(CH₂)_{q1}N⁺R₁R₂R₃, or (Aa)ᵣ;
X₁, X₂, X₄, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), CH₂, CHNH, CH₂O, C(O)NHNHC(O), OCH₂C₆H₄NH, NHCH₂C₆H₄NH, SCH₂C₆H₄NH and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; In addition, Y₂ can be O, O₂, NR₁, NH, or absent when it links S;
X₃ and Y₃ are independently N, NH, CH, CH₂ or CR₁, or one of X₃ and Y₃ can be absent; wherein R₁ is C₁-C₈ linear or branched alkyl, heteroalkyl; C₃-C₈ aryl, heteroaryl, alkylcycloalkyl, acyloxyl, alkylaryl, alkylaryloxyl, alkylarylamino, alkylarylthiol; (Ar)ᵣ, r=1-6 (amino acid or peptides having the same or different sequence of amino acids);
X₃ is H, CH₃ or X₁′ R₁′, wherein X₁′ is NH, N(CH₃), NHNH, O, or S; and R₁′ is H, C₁-C₈ linear or branched alkyl, C₃-C₈ aryl, heteroaryl, heteroalkyl, alkylcycloalkyl, acyloxylamines;
Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃;
Z₃ is H, OP(O)(OM₁)(OM₂), OCH₂OP(O)(OM₁)(OM₂), OSO₃M₁, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃;
X₅ is F, Cl, Br, I, OH, OR₁, R₁, OPO₃H₂, OSO₃H, NHR₁, OCOR₁, NHCOR₁, CN or OCH₂OP(O)(OM₁)(OM₂);
Y₅ is NH, NHNH, NR₁, O, OCH₂C₆H₄NH, NHCH₂C₆H₄NH, SCH₂C₆H₄NH, R₁, (Ar)ᵣ, r=1-6 (amino acid or peptides having the same or different sequence of amino acids);
X₆ and Y₆ are independently CH, C(O), N, P(O)NH, P(O)NR₁, CHC(O)NH, C₁-C₈ linear or branched alkyl, or heteroalkyl; C₃-C₈ aryl, heteroaryl, alkylcycloalkyl, acyloxyl, alkylaryl, alkylaryloxyl, alkylarylamino, or an Aa (amino acid, preferably selected from Lys, Phe, Asp, Glu, Ser, Thr, His, Cys, Tyr, Trp, Gln, Asn, Arg);
Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) or C(O)NR₁;
X₈ is O, S, NH, NHNH, NHR₁, SR₁₂, SSR₁₂, SSCH(CH₃)R₁, SSC(CH₃)₂R₁, or R₁; wherein R₁, R₂ and R₃ are in dependently H, C₁-C₈ linear or branched alkyl, C₃-C₈ aryl, heteroaryl, heteroalkyl, alkylcycloalkyl, acyloxylamines;
Lv₁ is a leaving group defined the same above. Preferably Lv₁ is selected from F, Cl, Br, I, OTs, OMS, OC₆H₃(NO₂)₂, OC₆F₅, OC₆H₄(NO₂), OC₆Cl₅;
M₁ and M₂ are independently H, Na, K, Ca, Mg, NH₄, NR₁R₂R₃; “ [symbol] ” is defined the same above.

Examples of preferred drug/linker complex of formula (I), (II), and (III) which can be used under the process of the invention to achieve over 80% of the total drugs linked to the cysteines between heavy-light chains of the antibody are illustrated below:

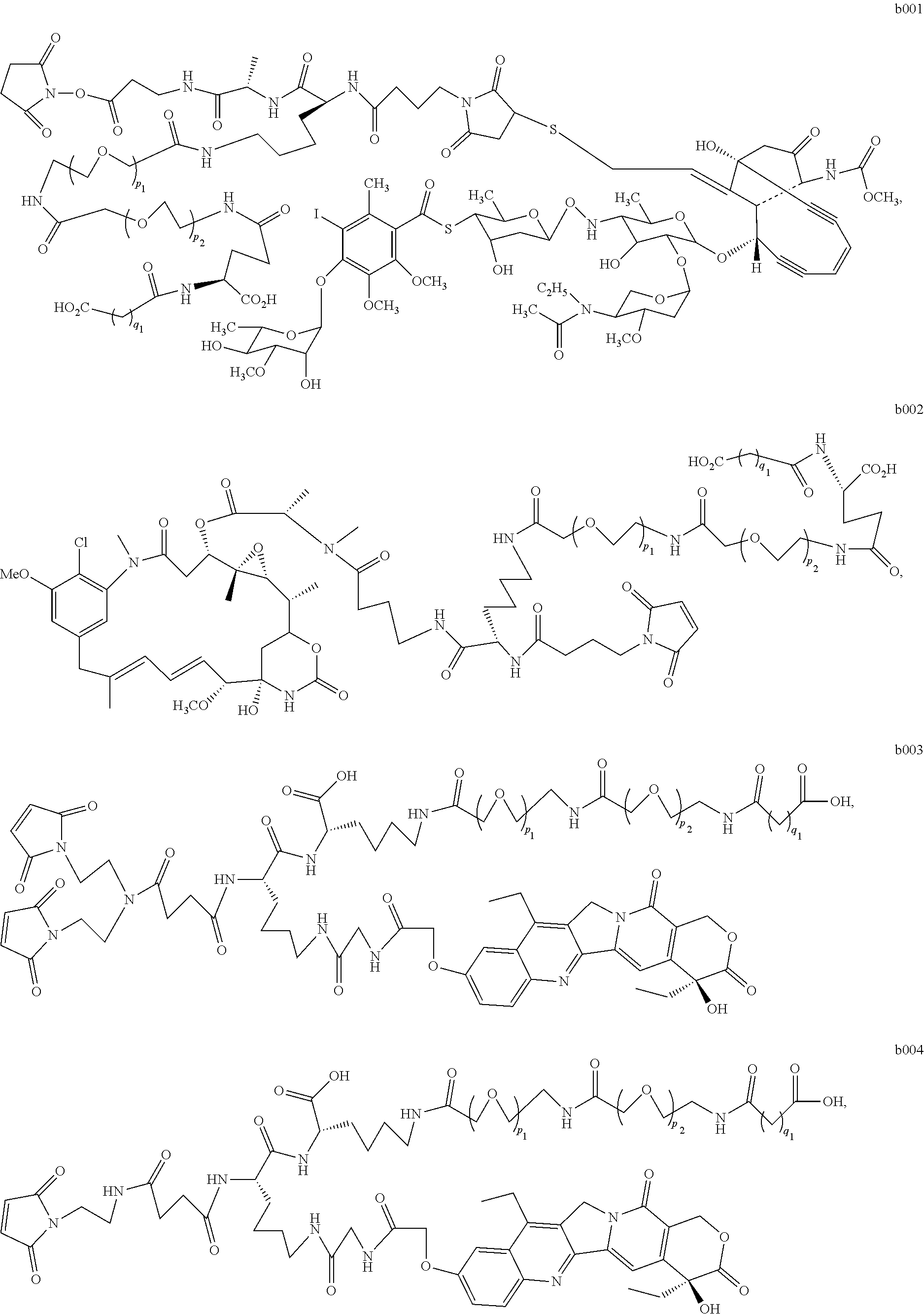

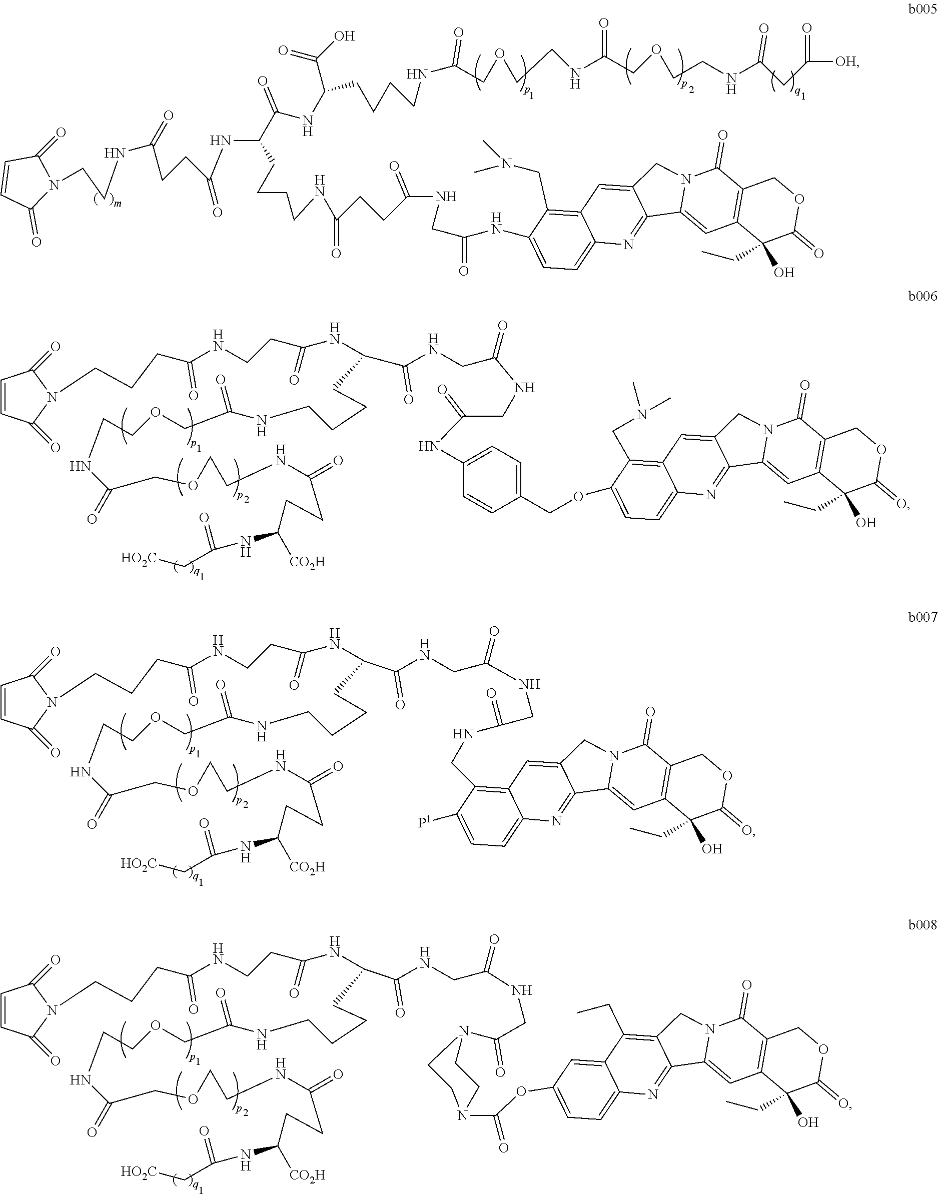

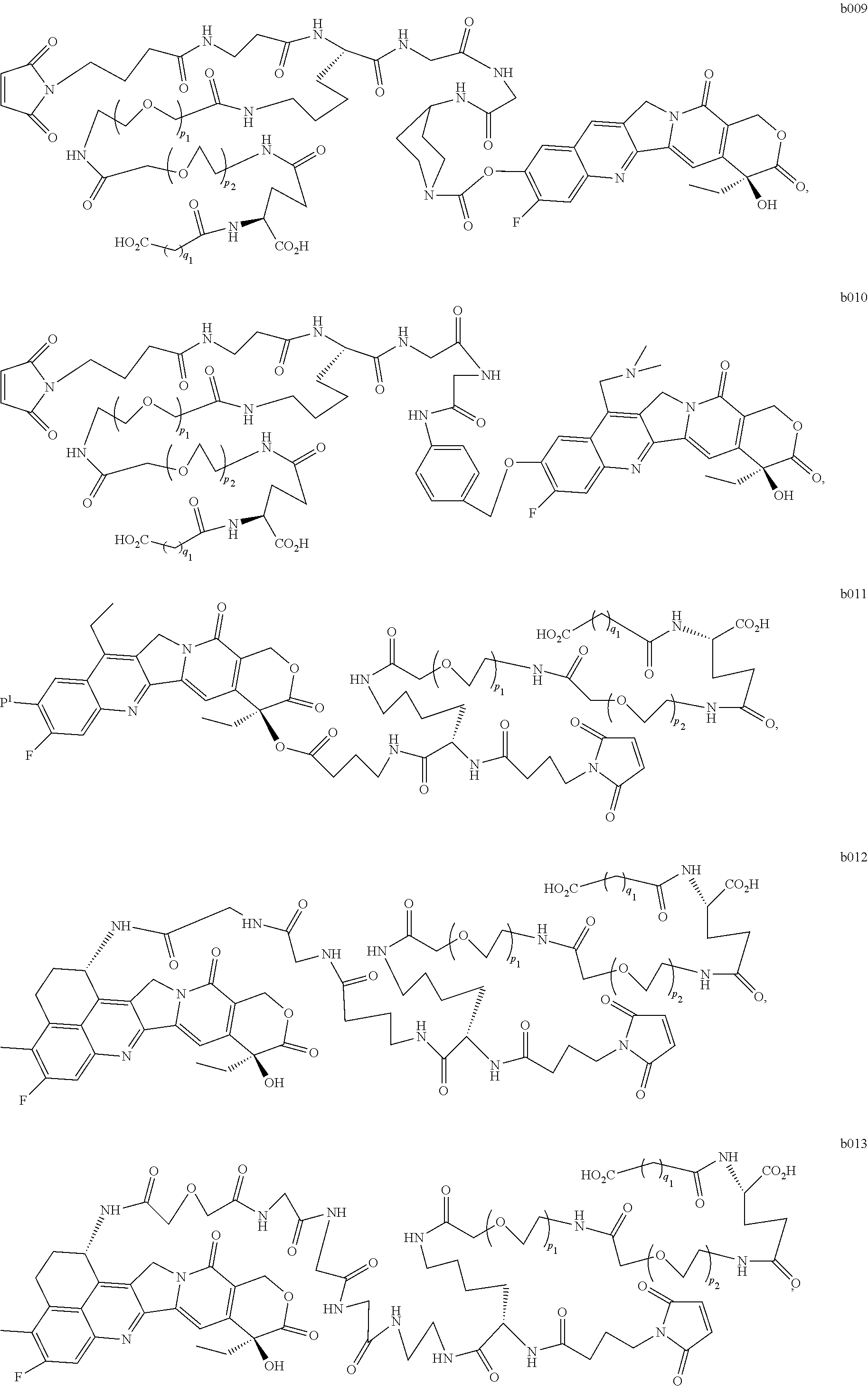

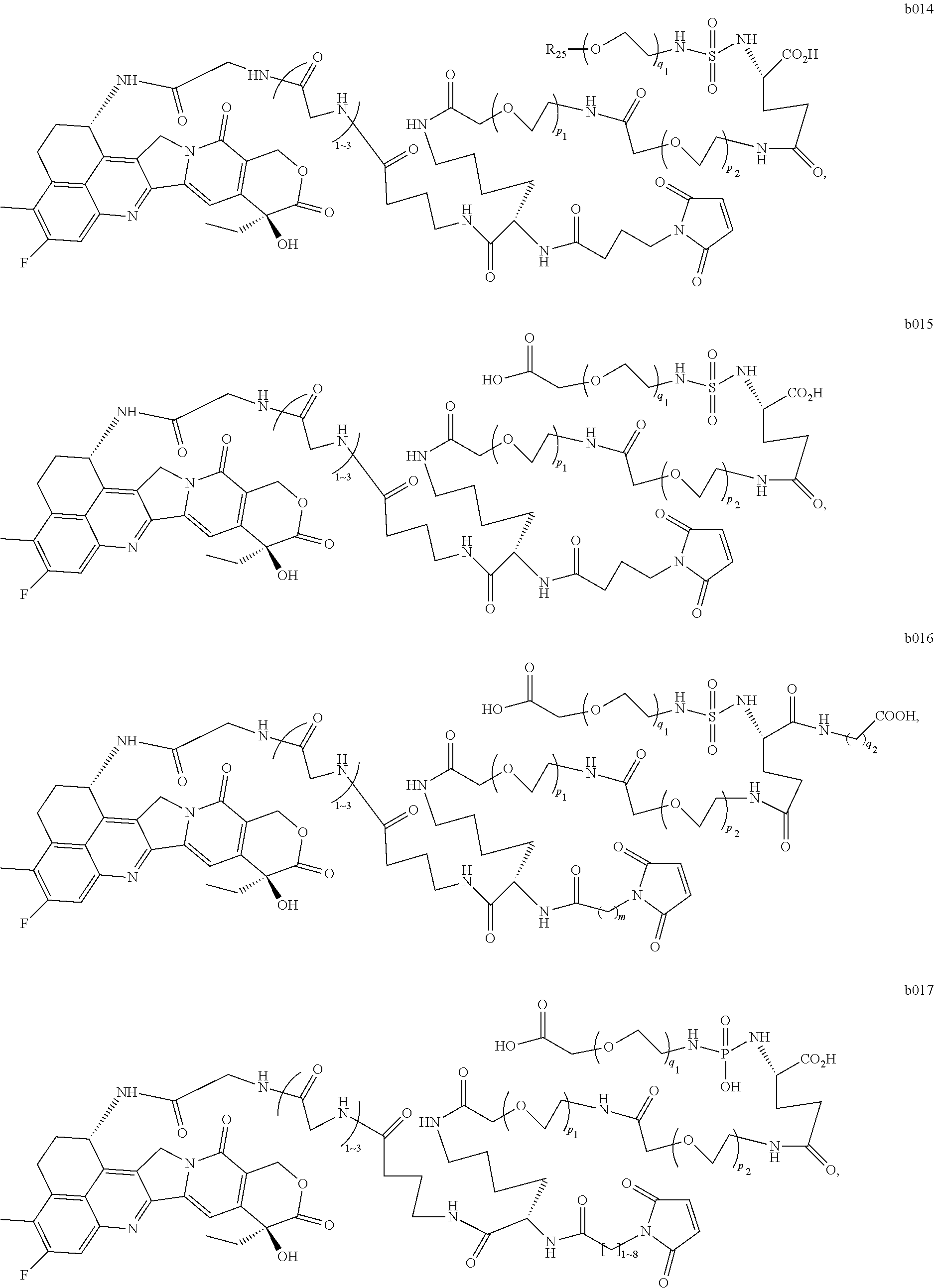

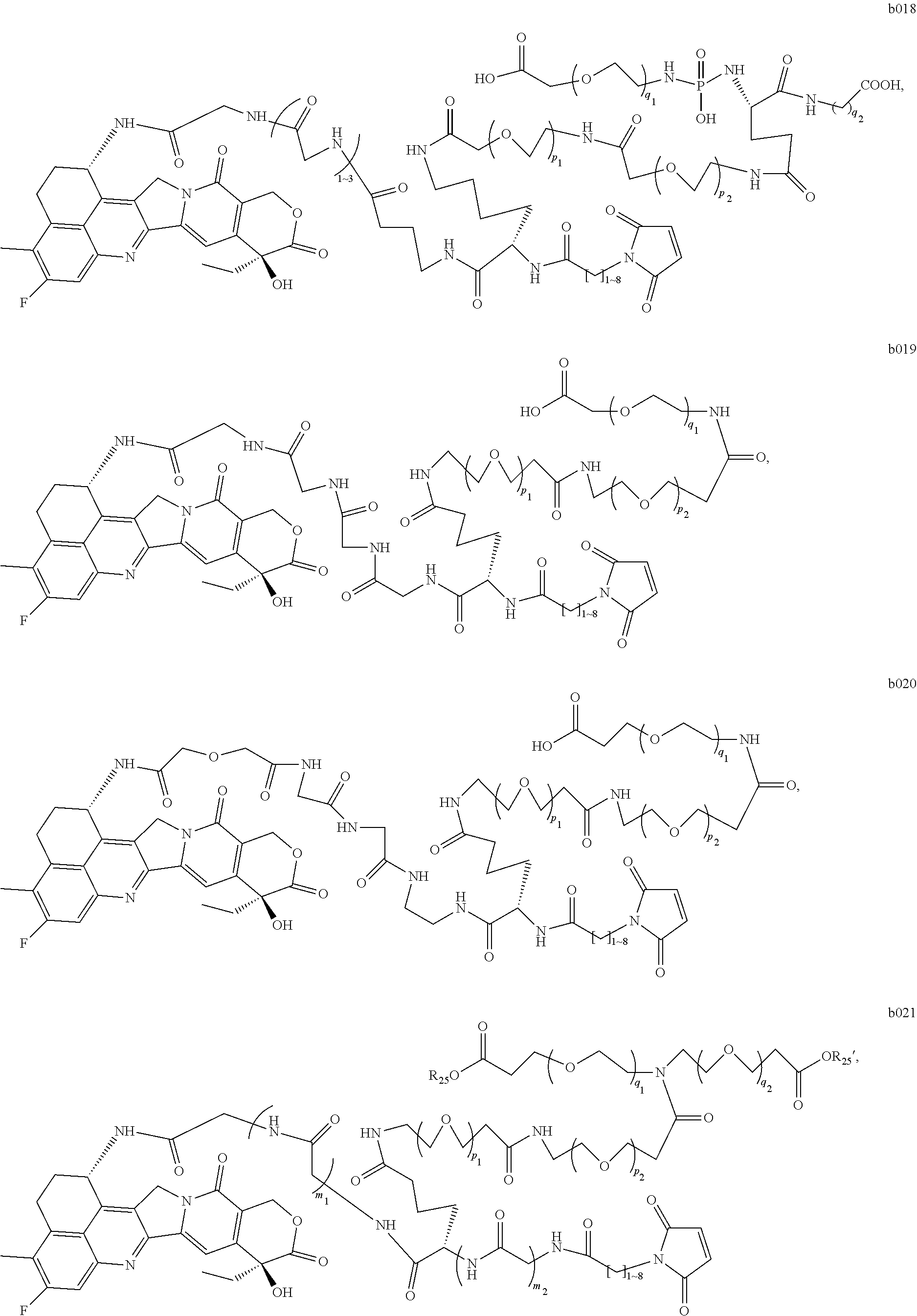

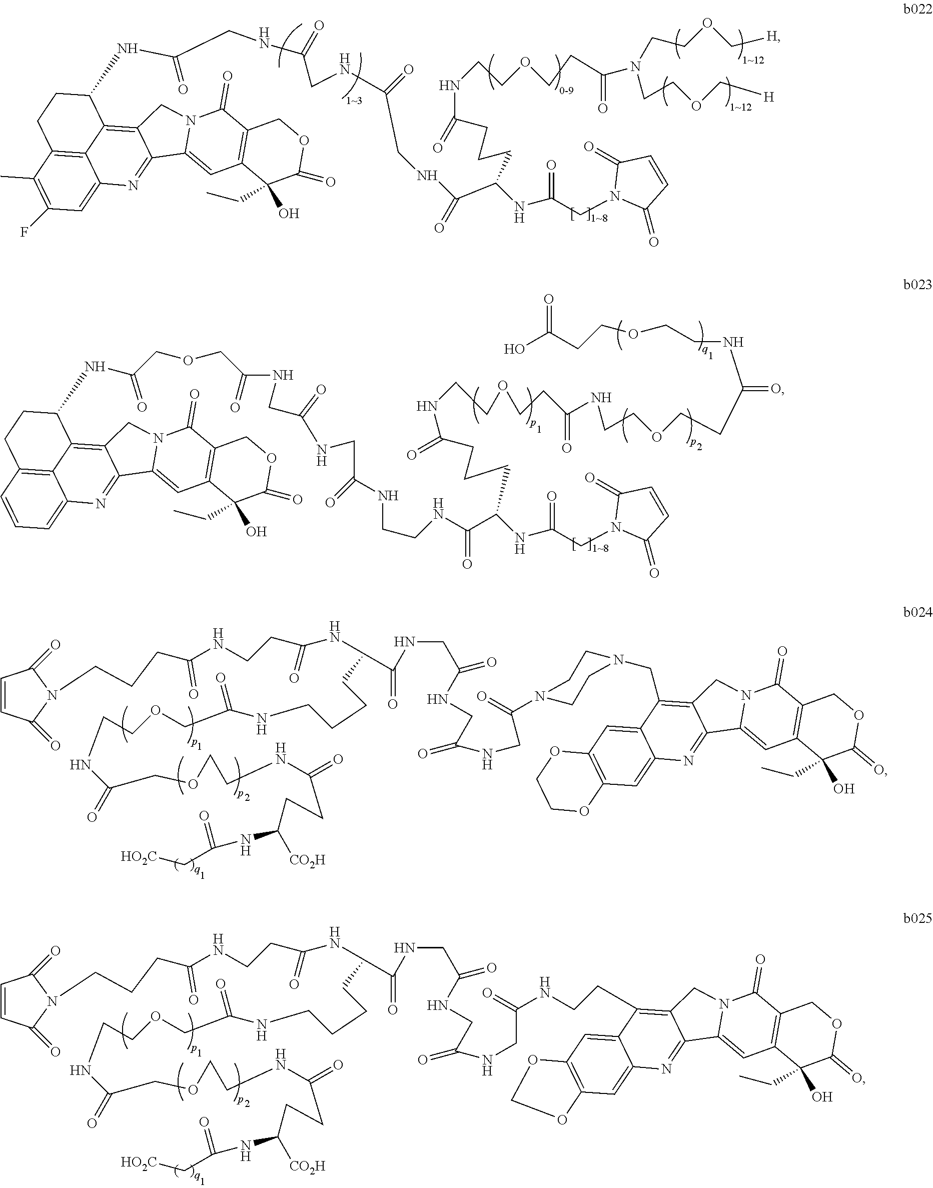

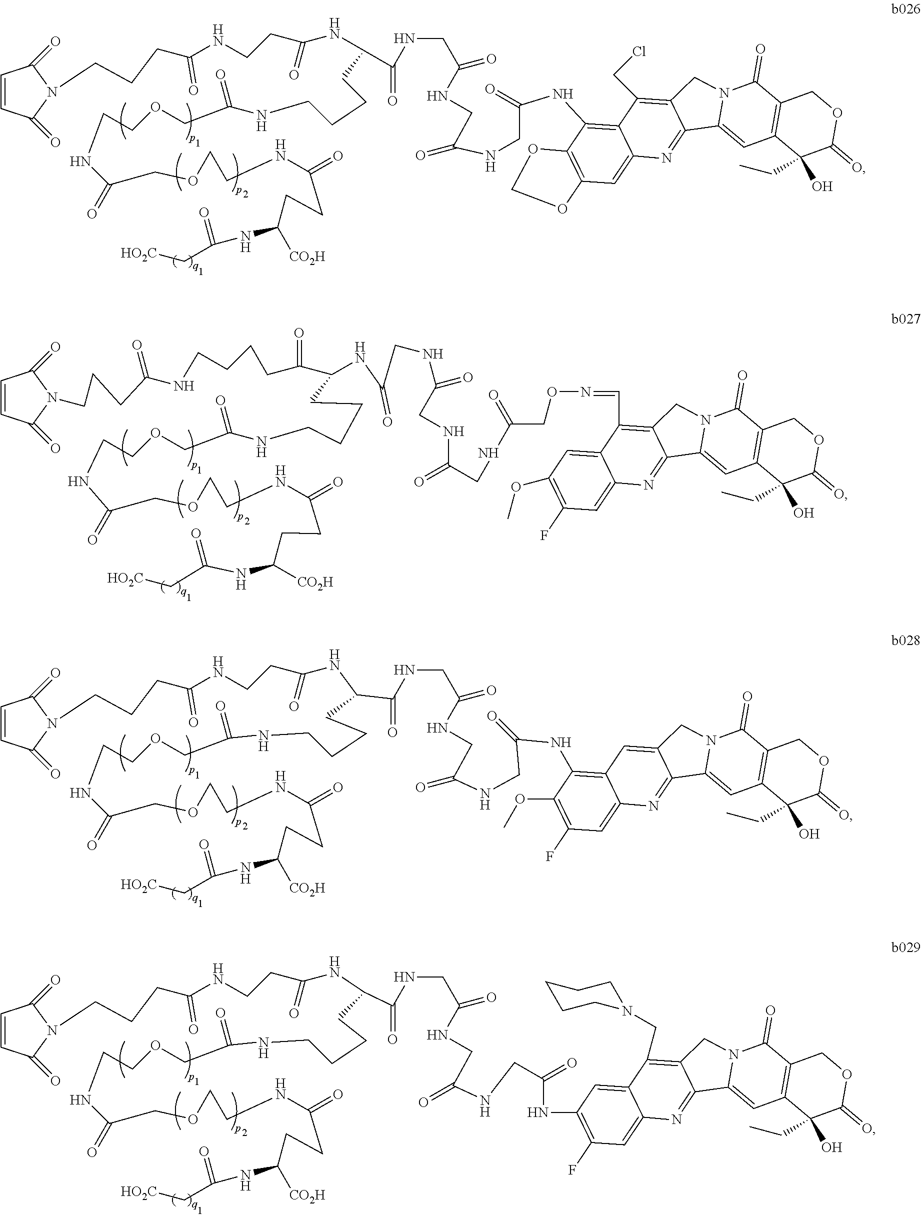

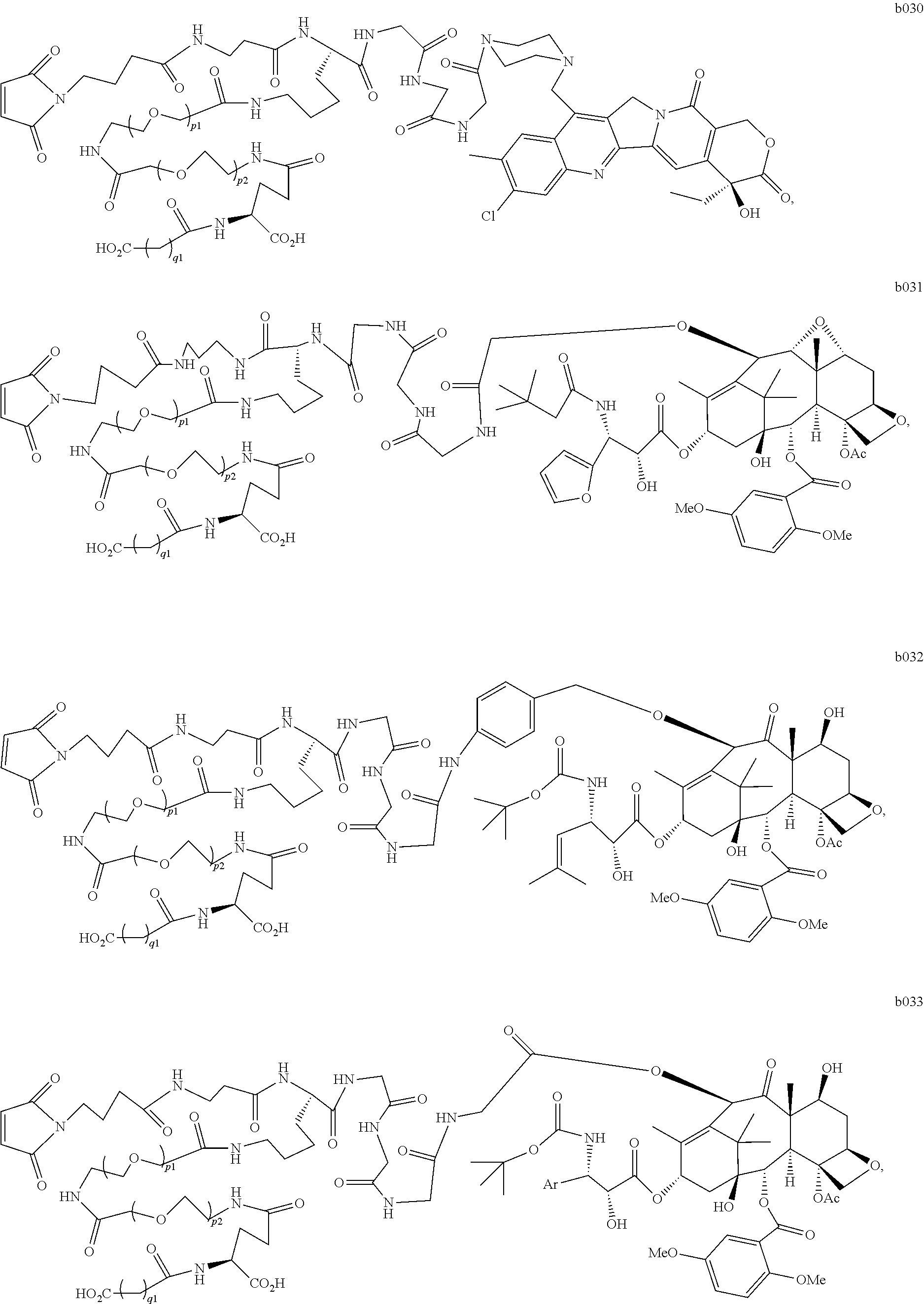

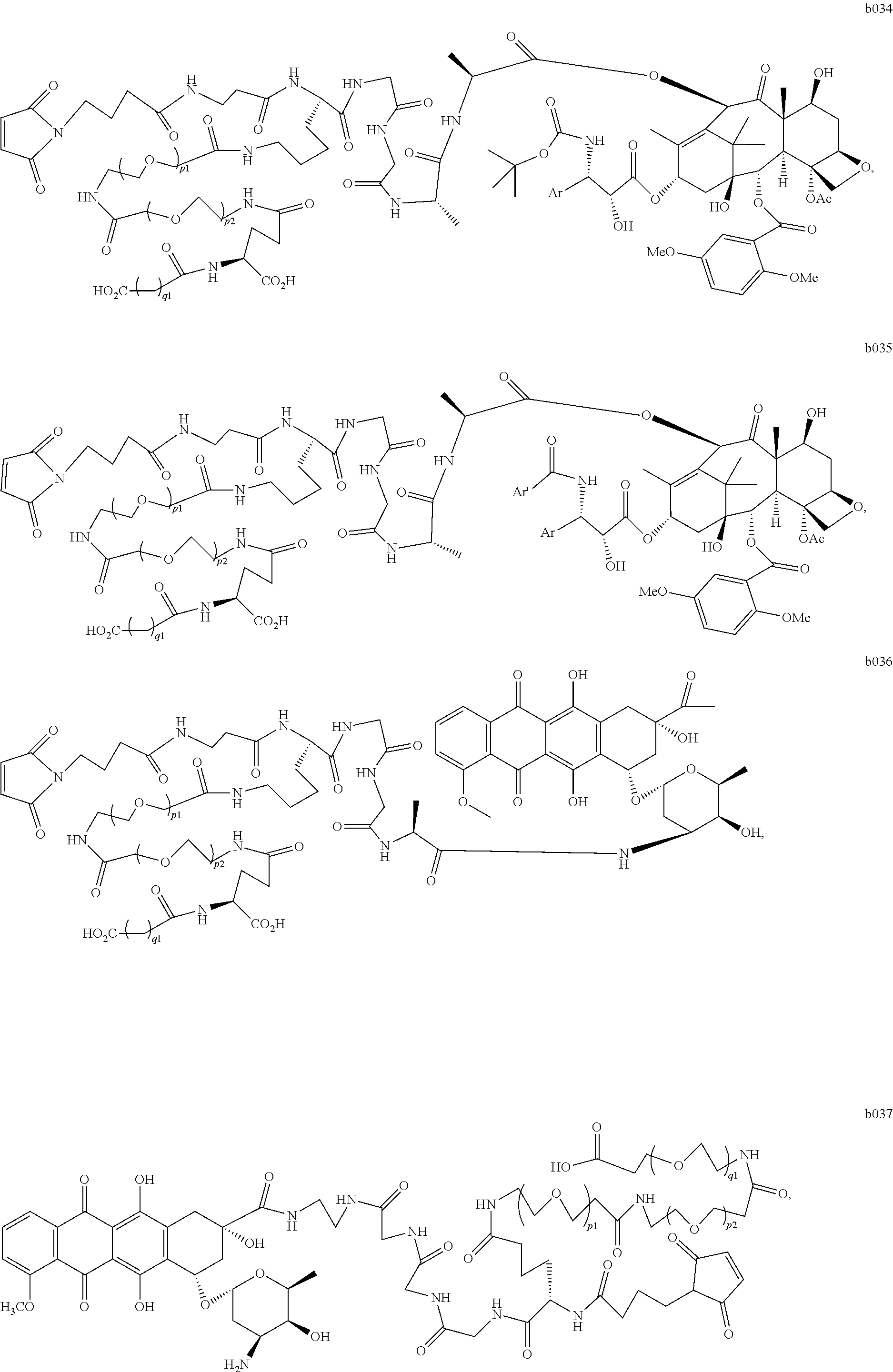

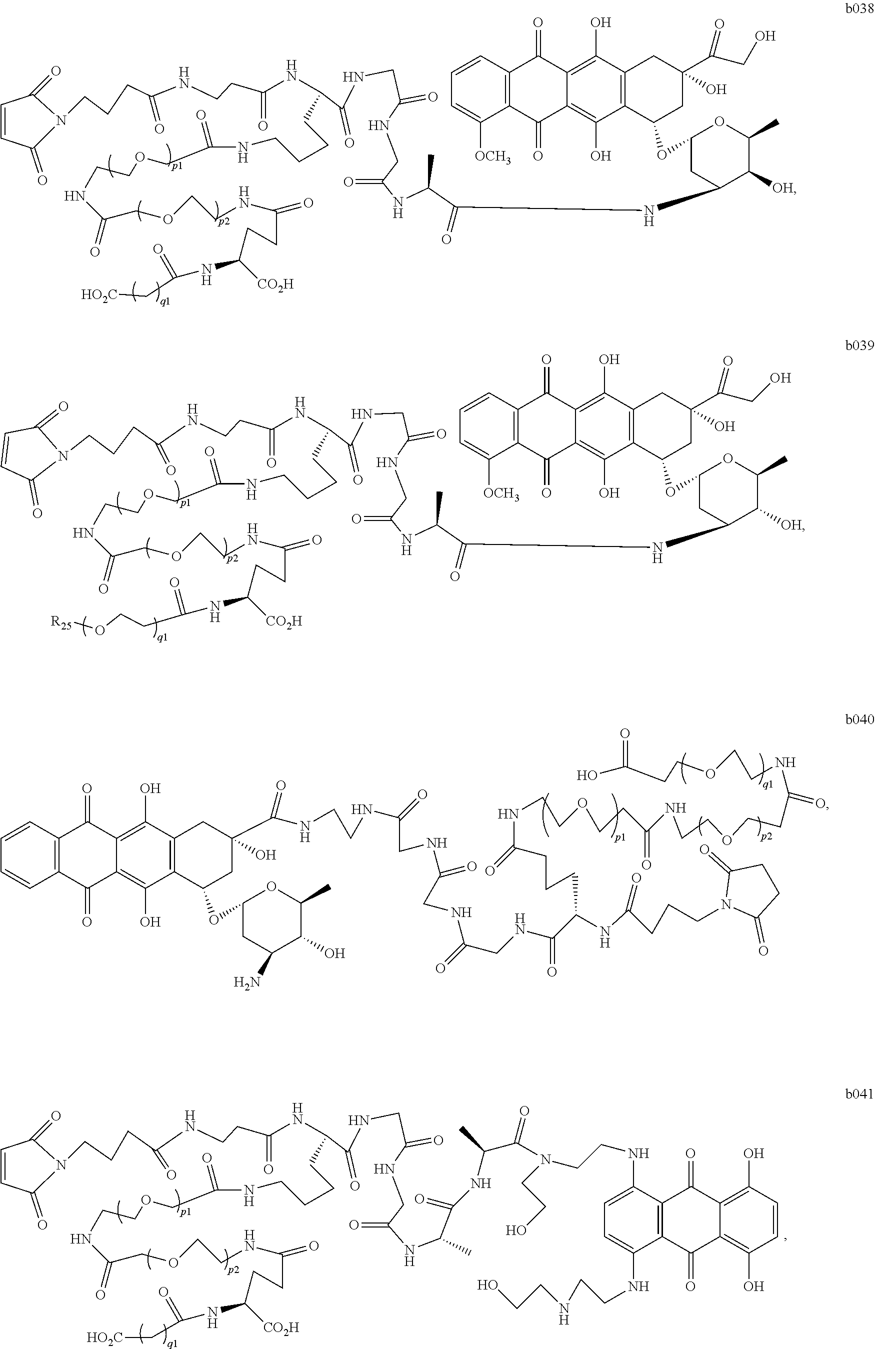

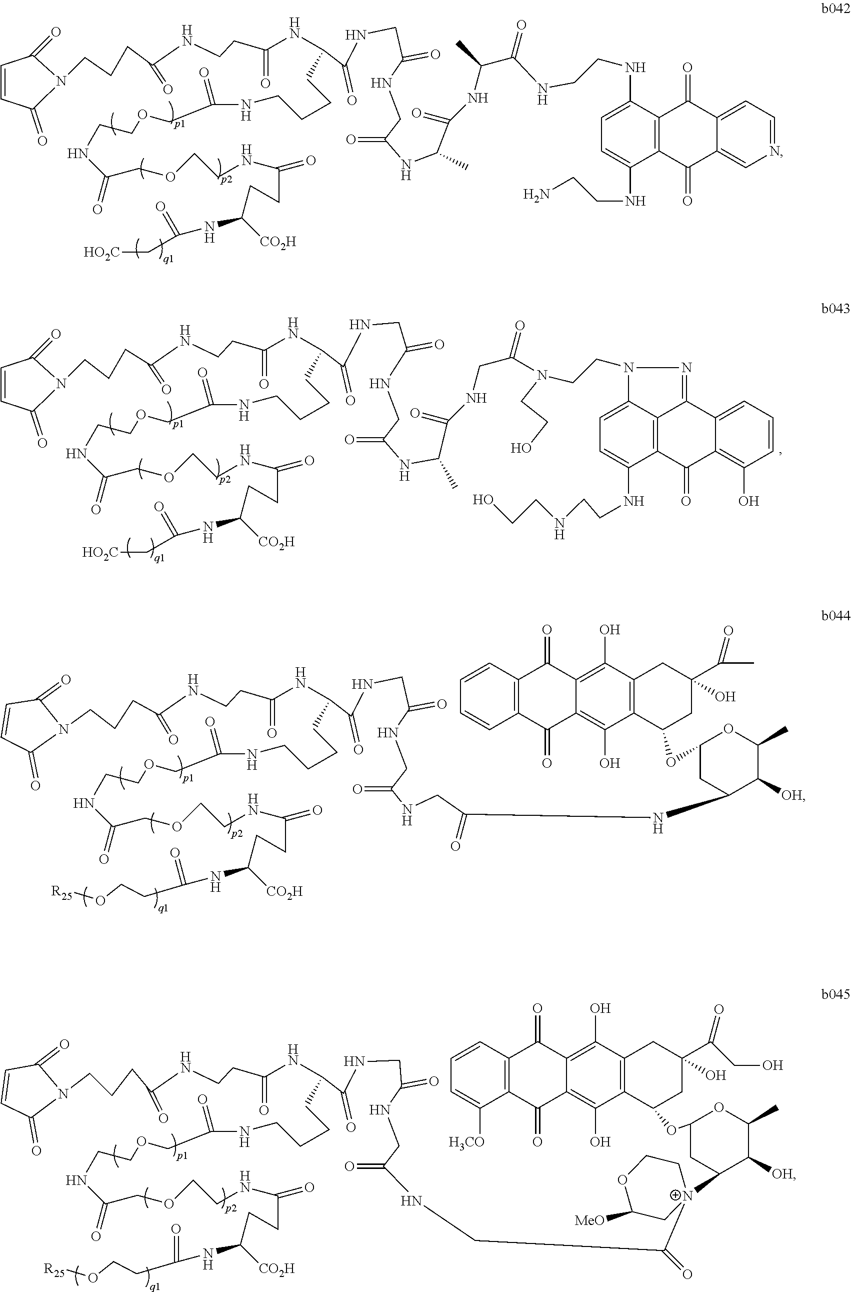

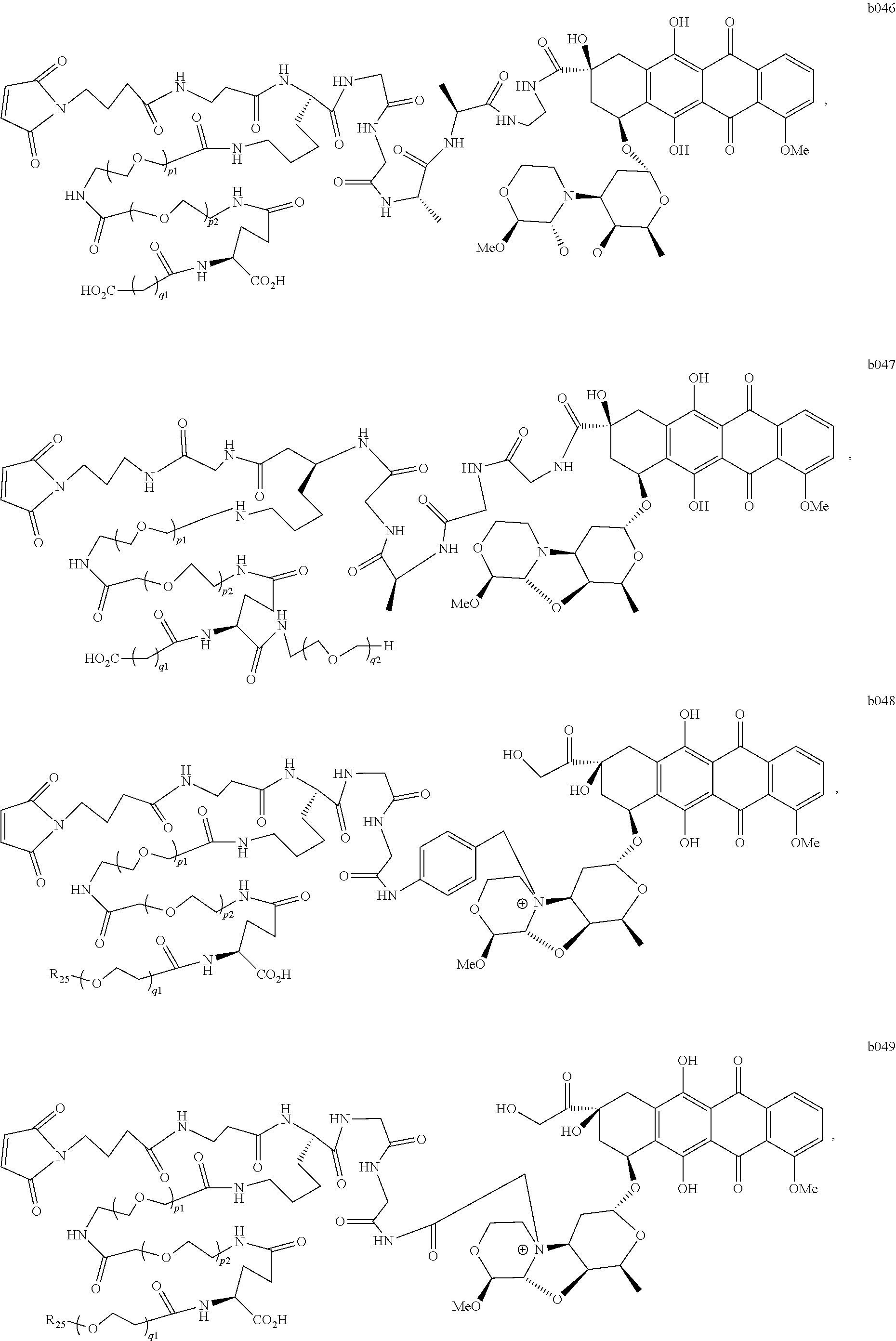

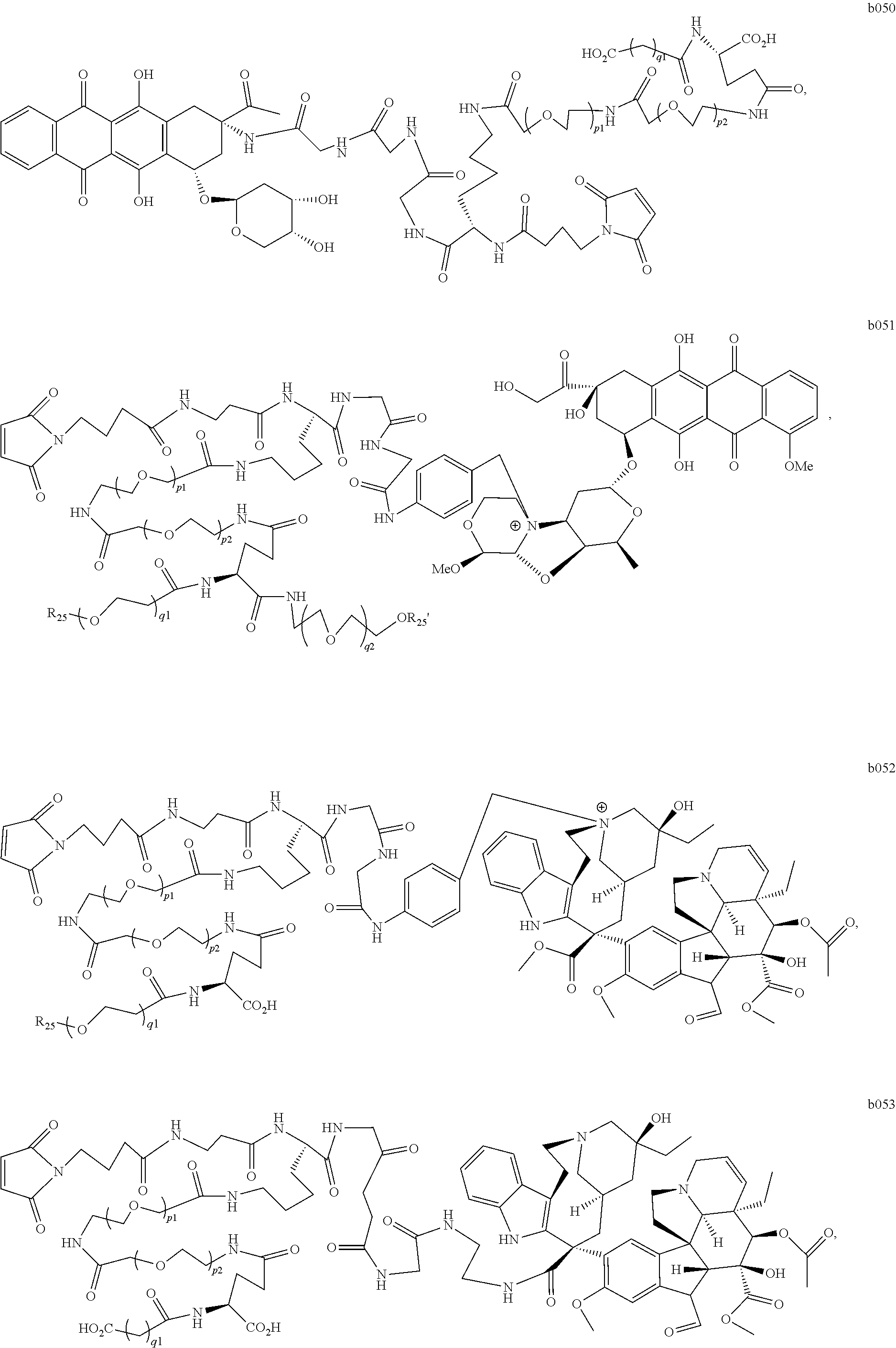

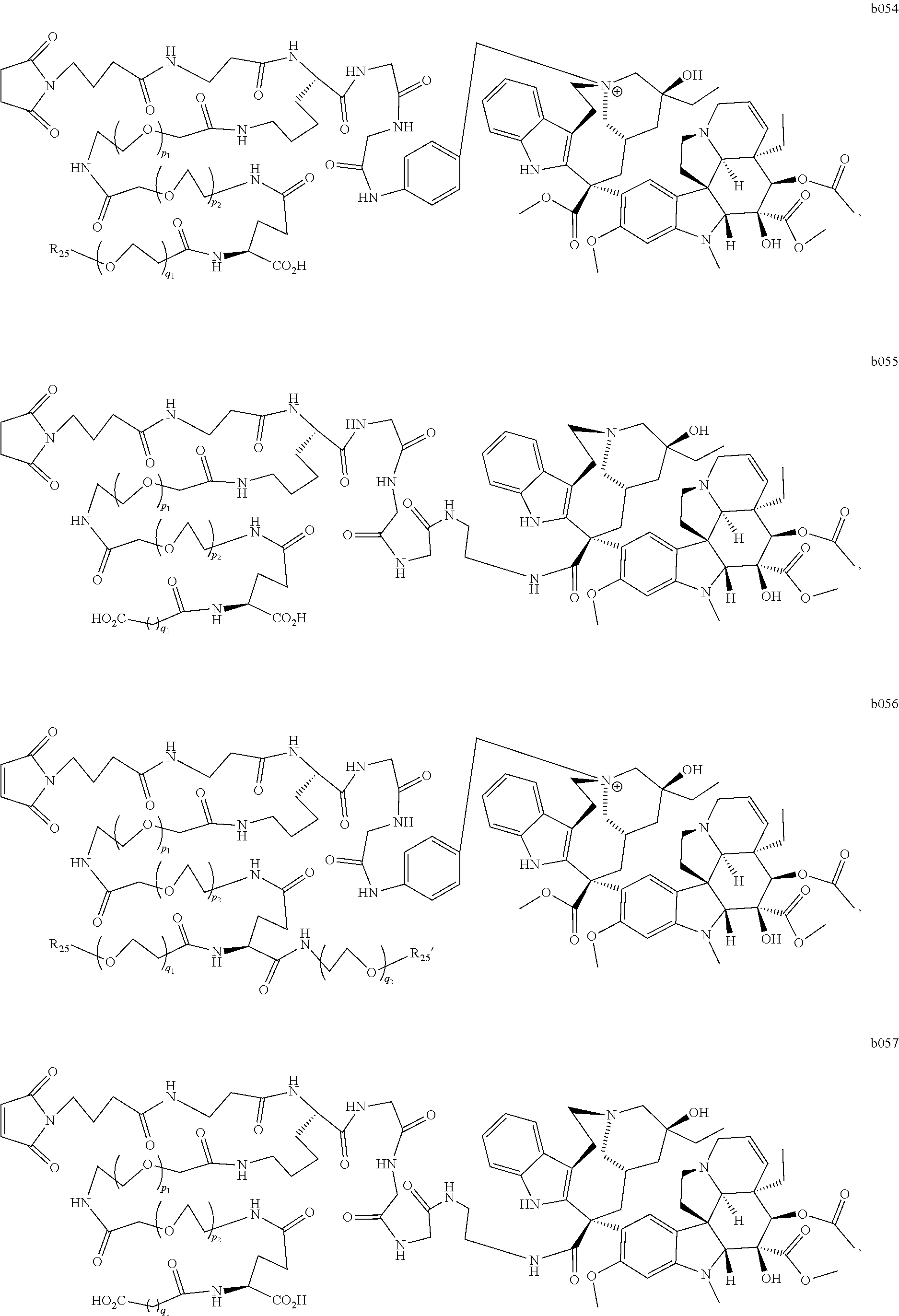

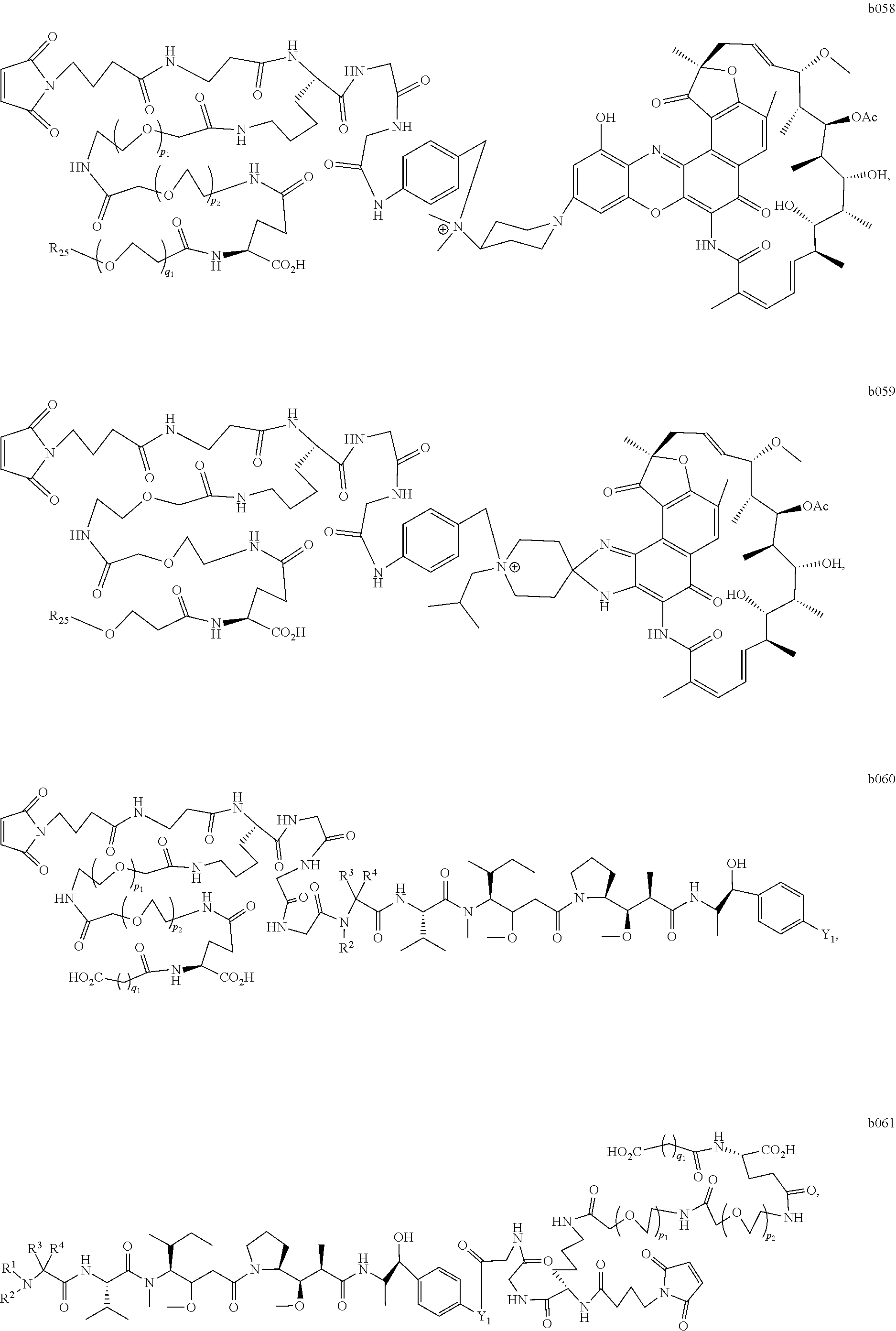

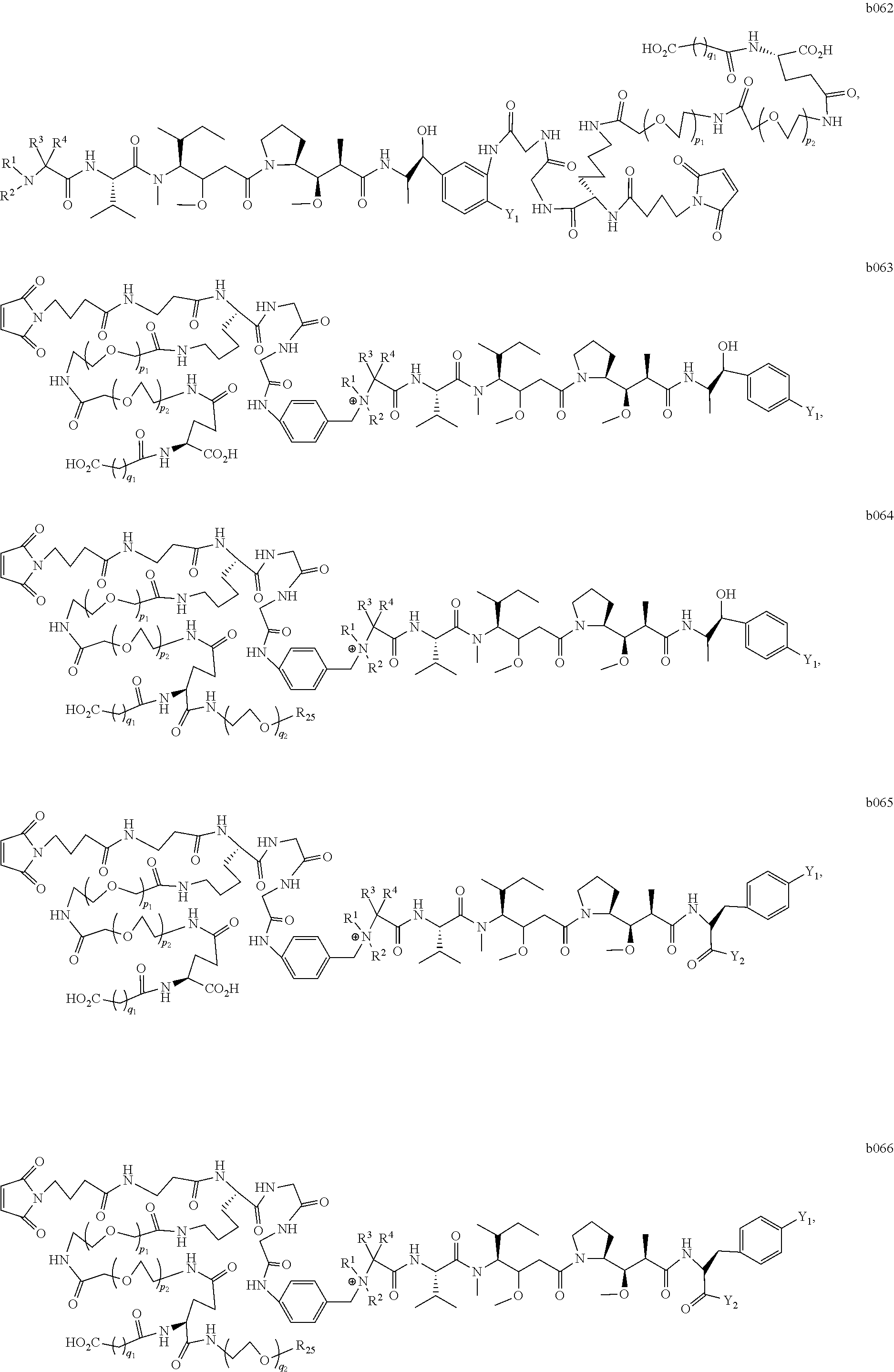

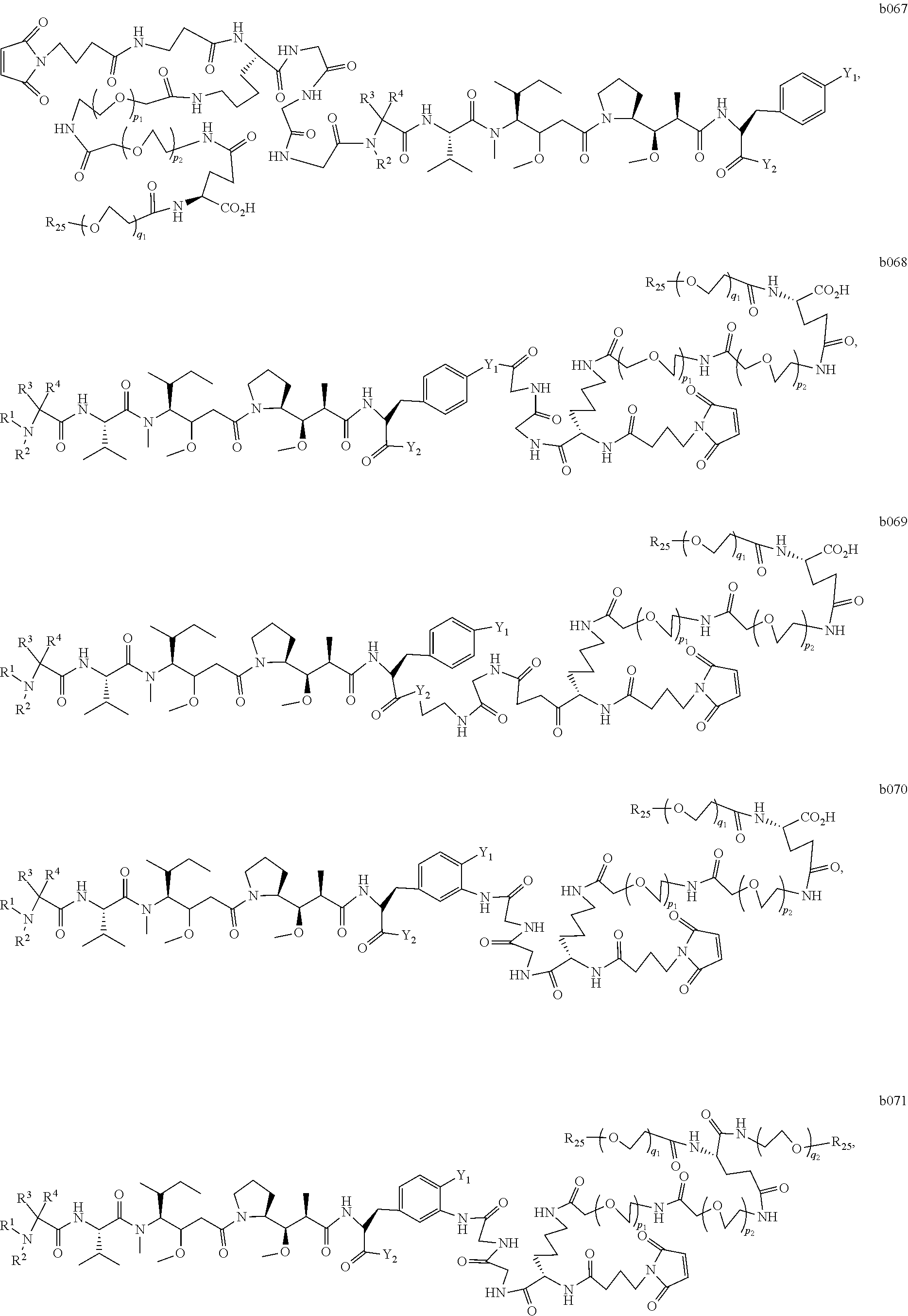

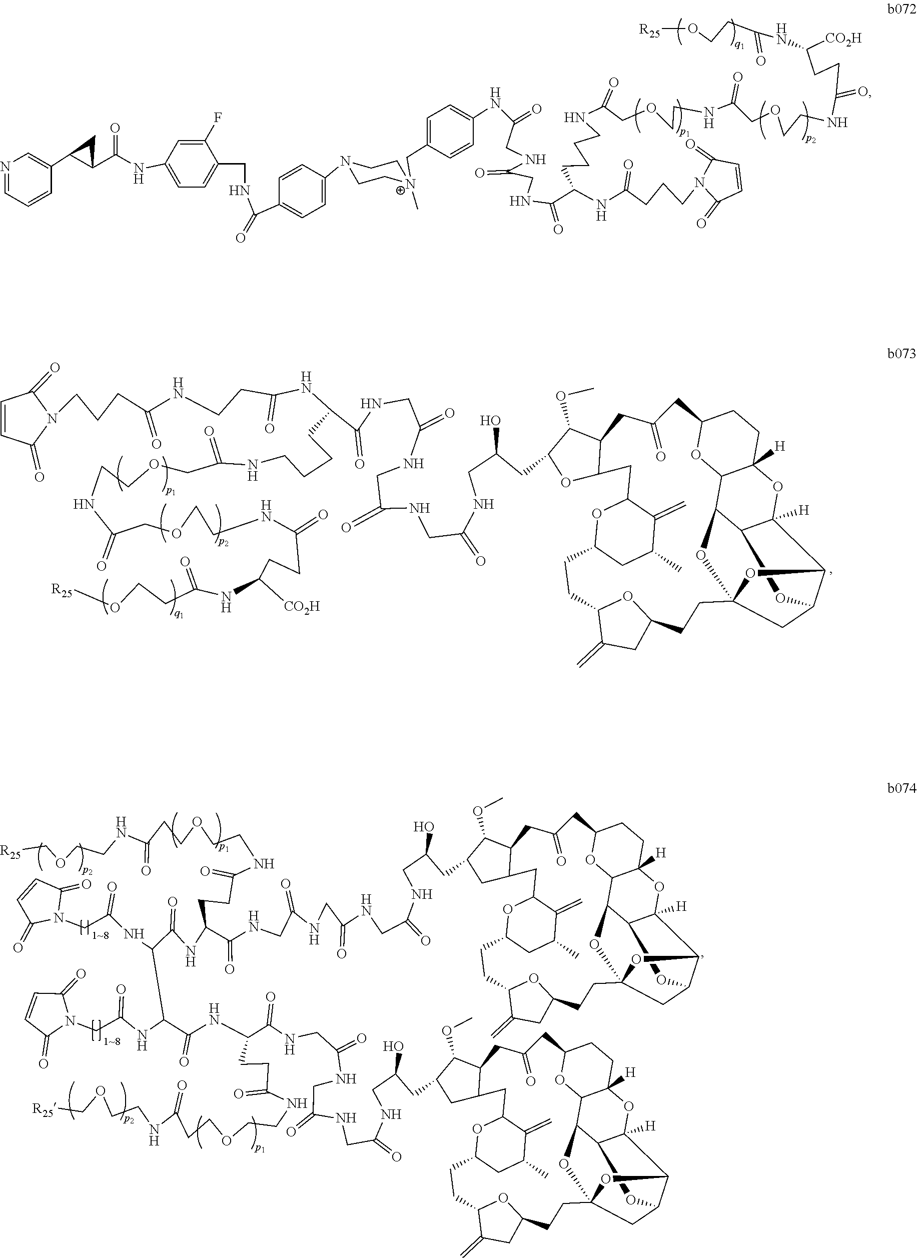

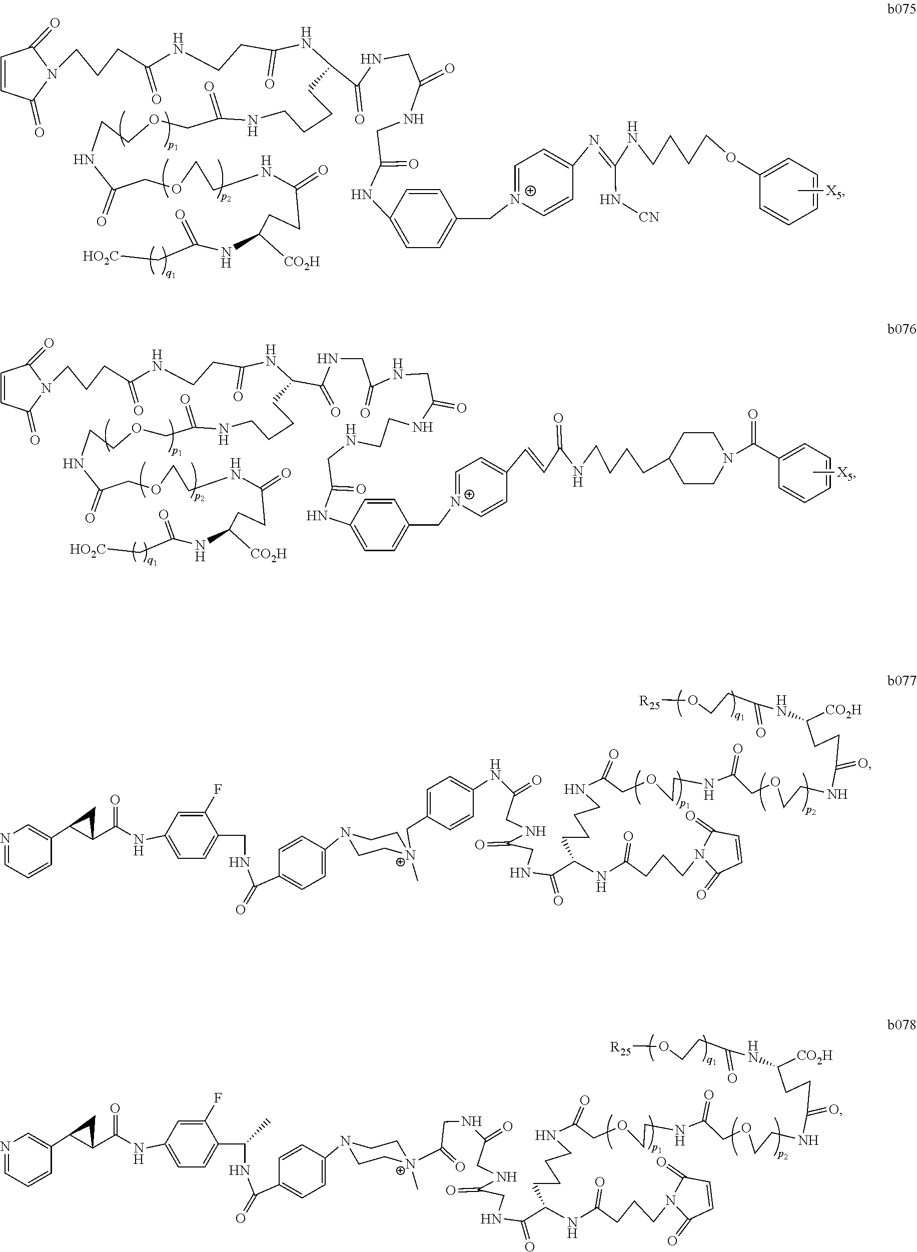

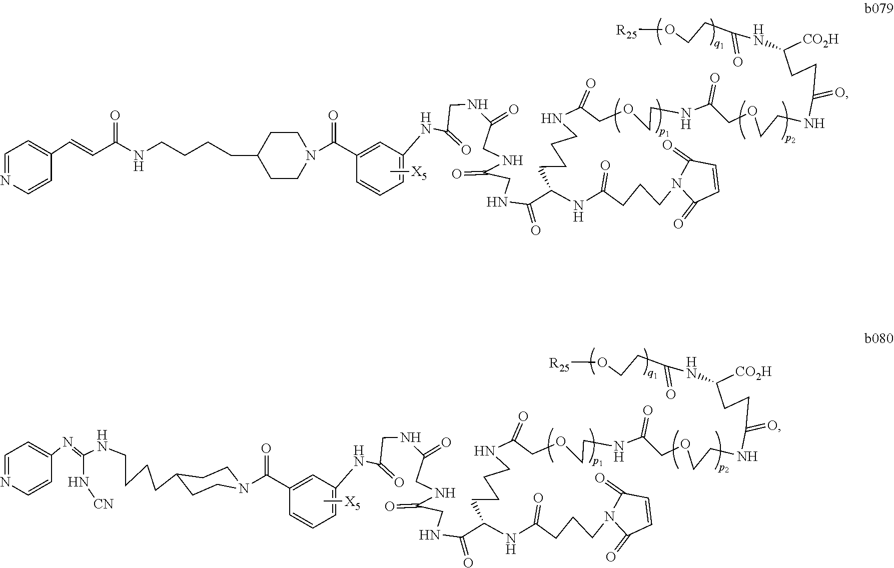

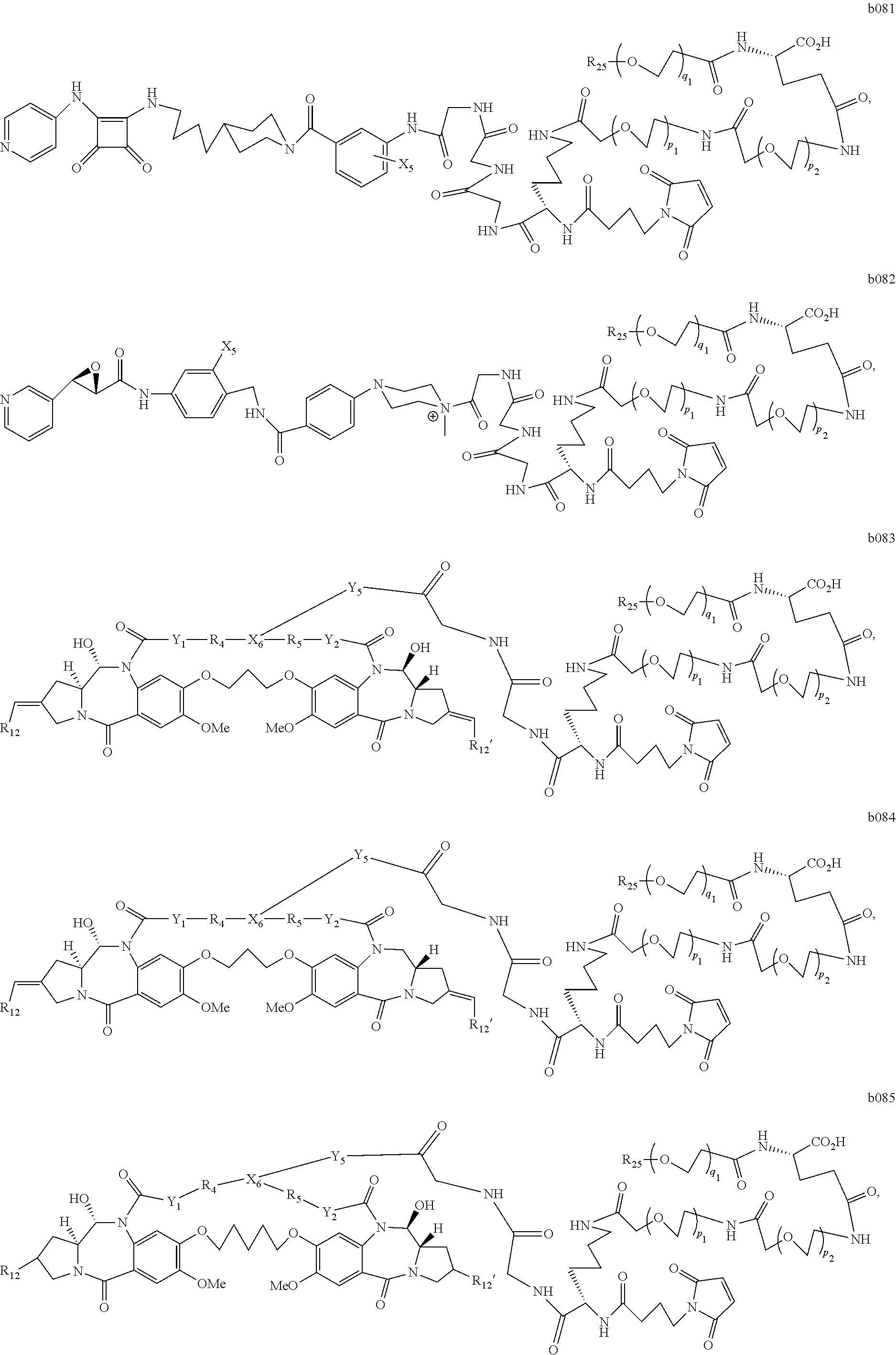

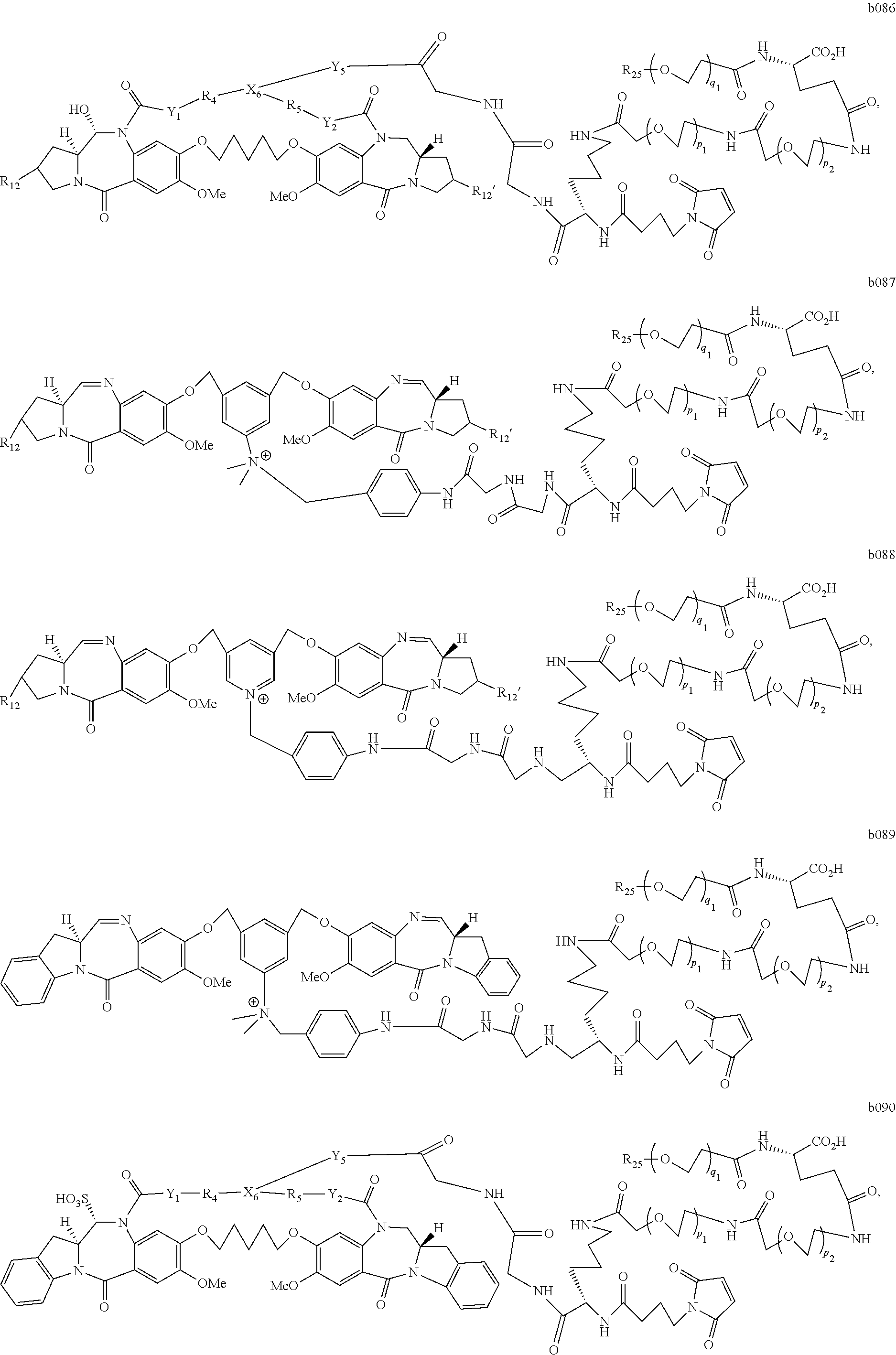

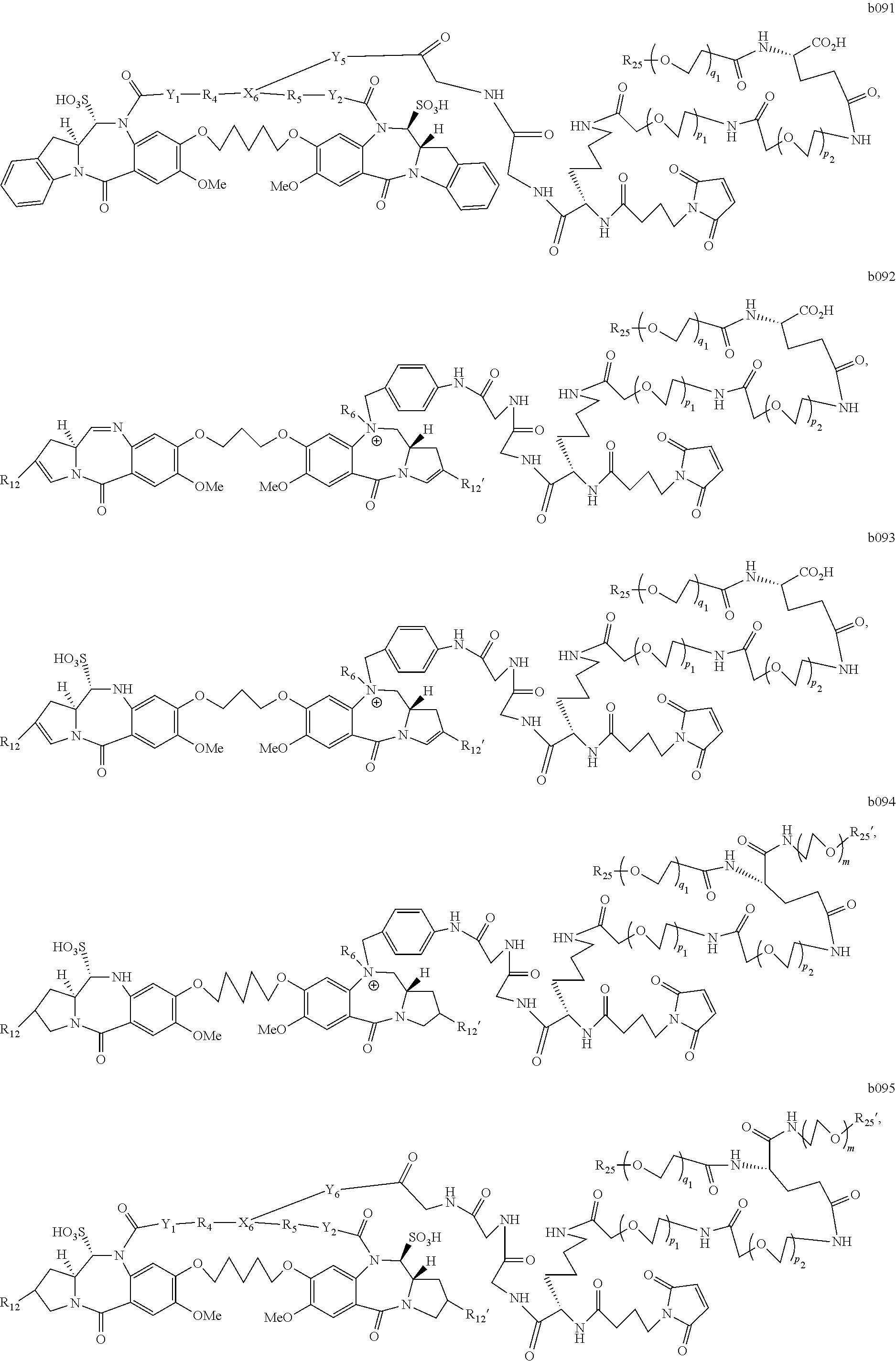

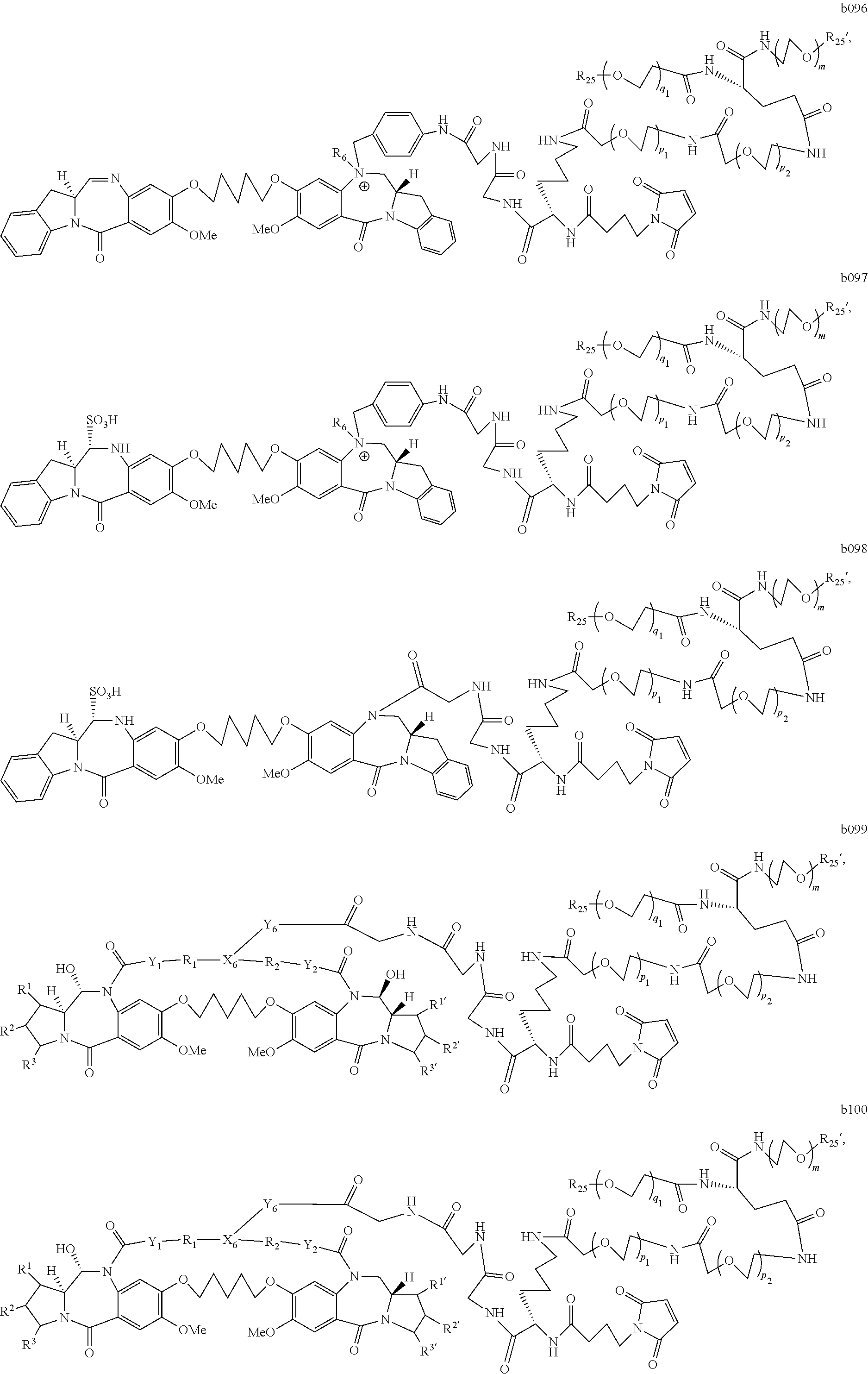

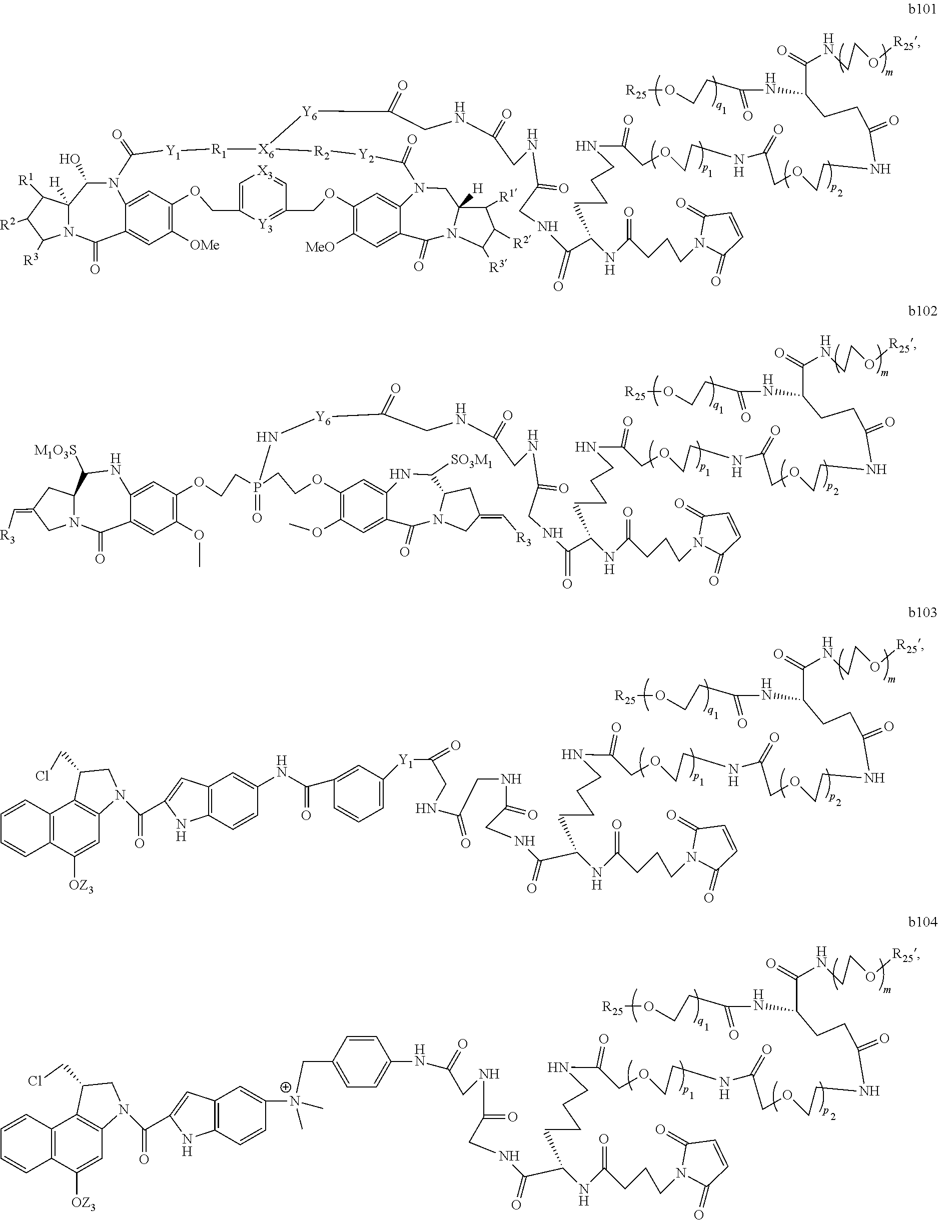

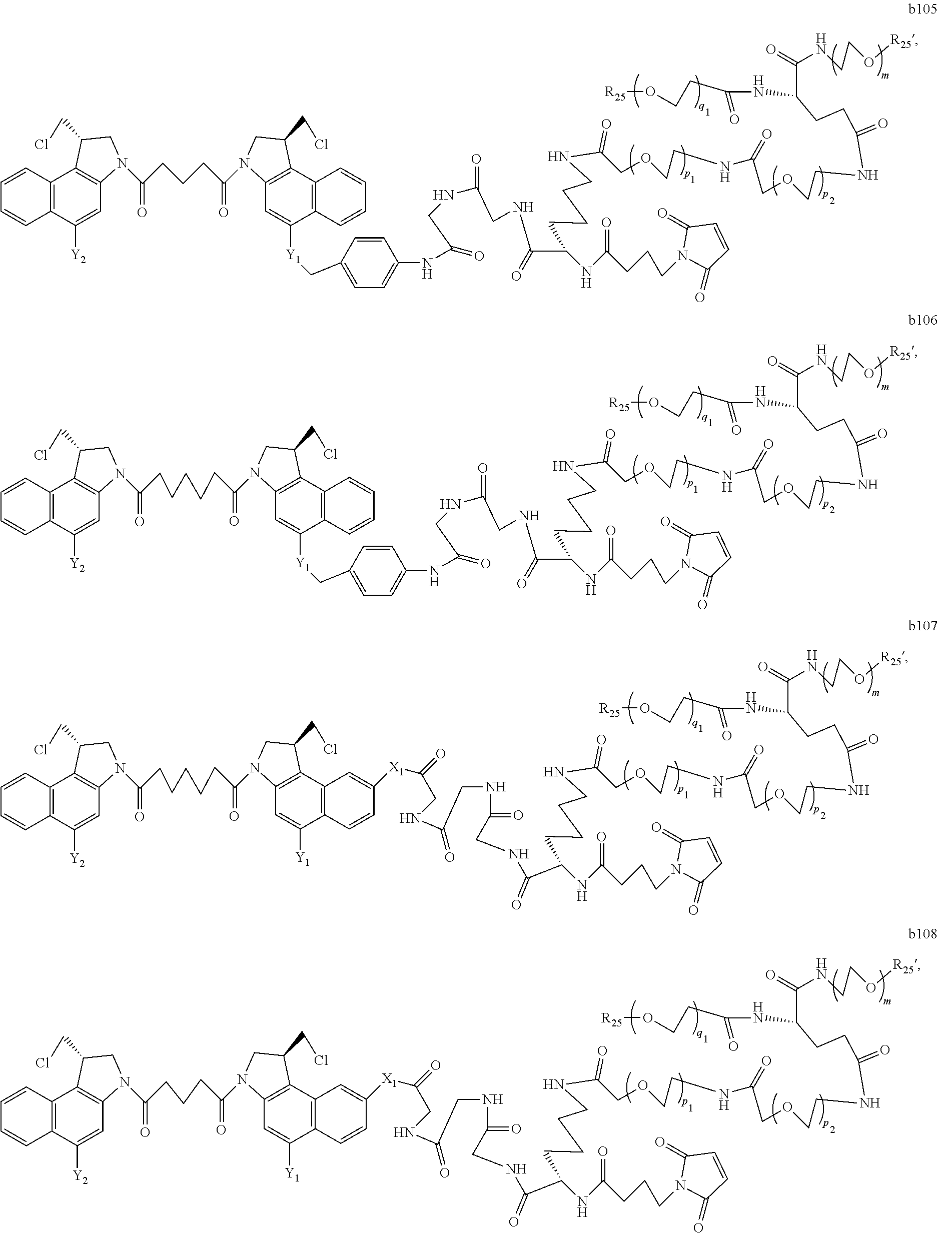

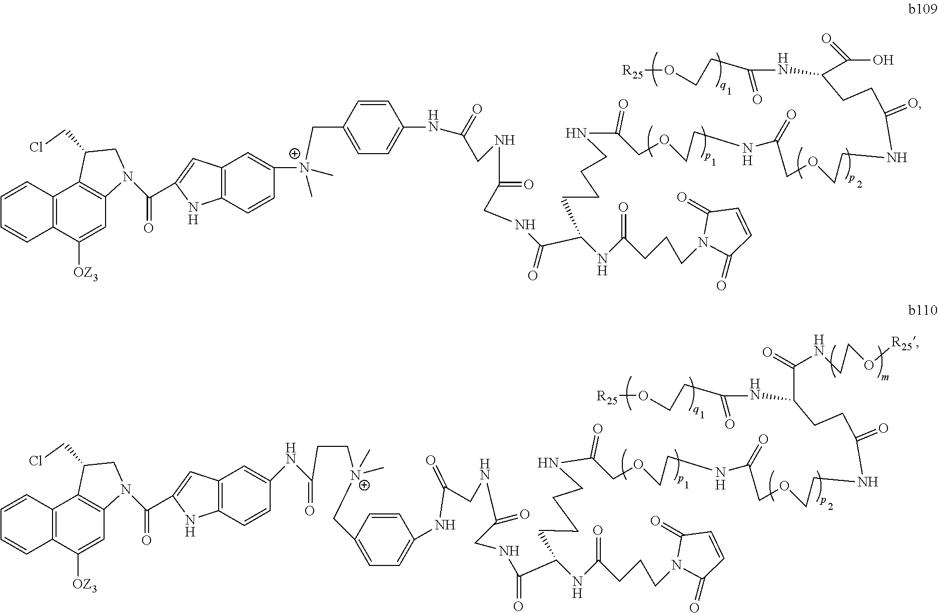

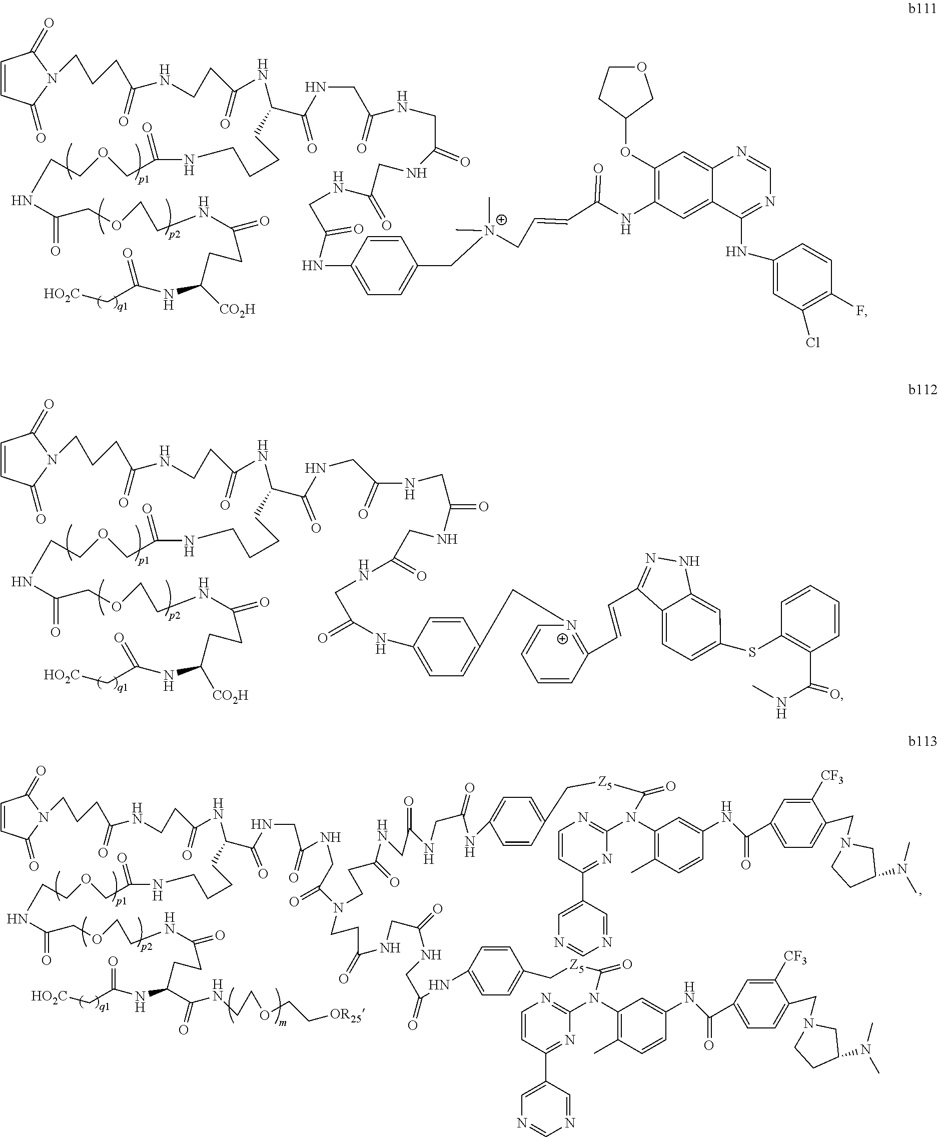

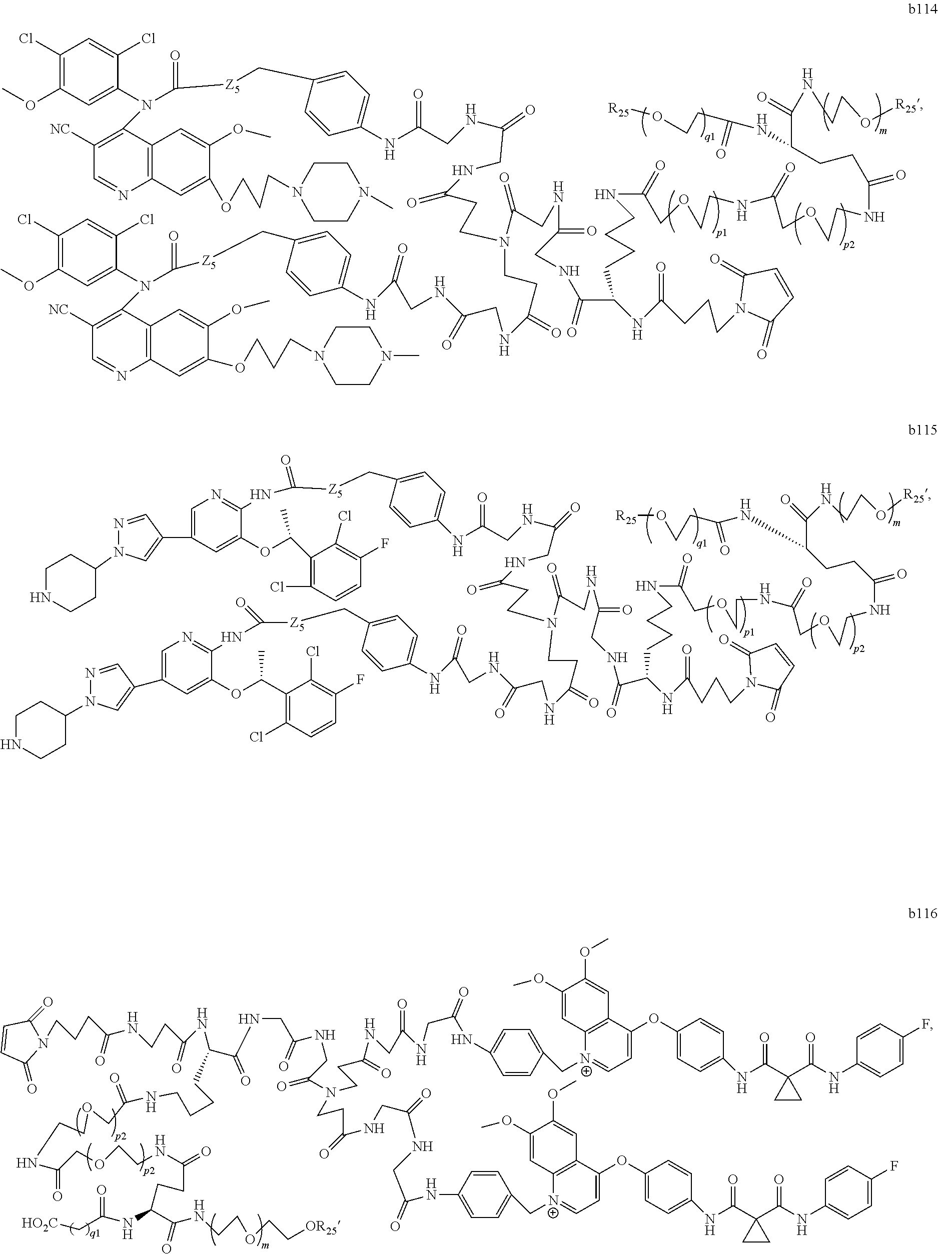

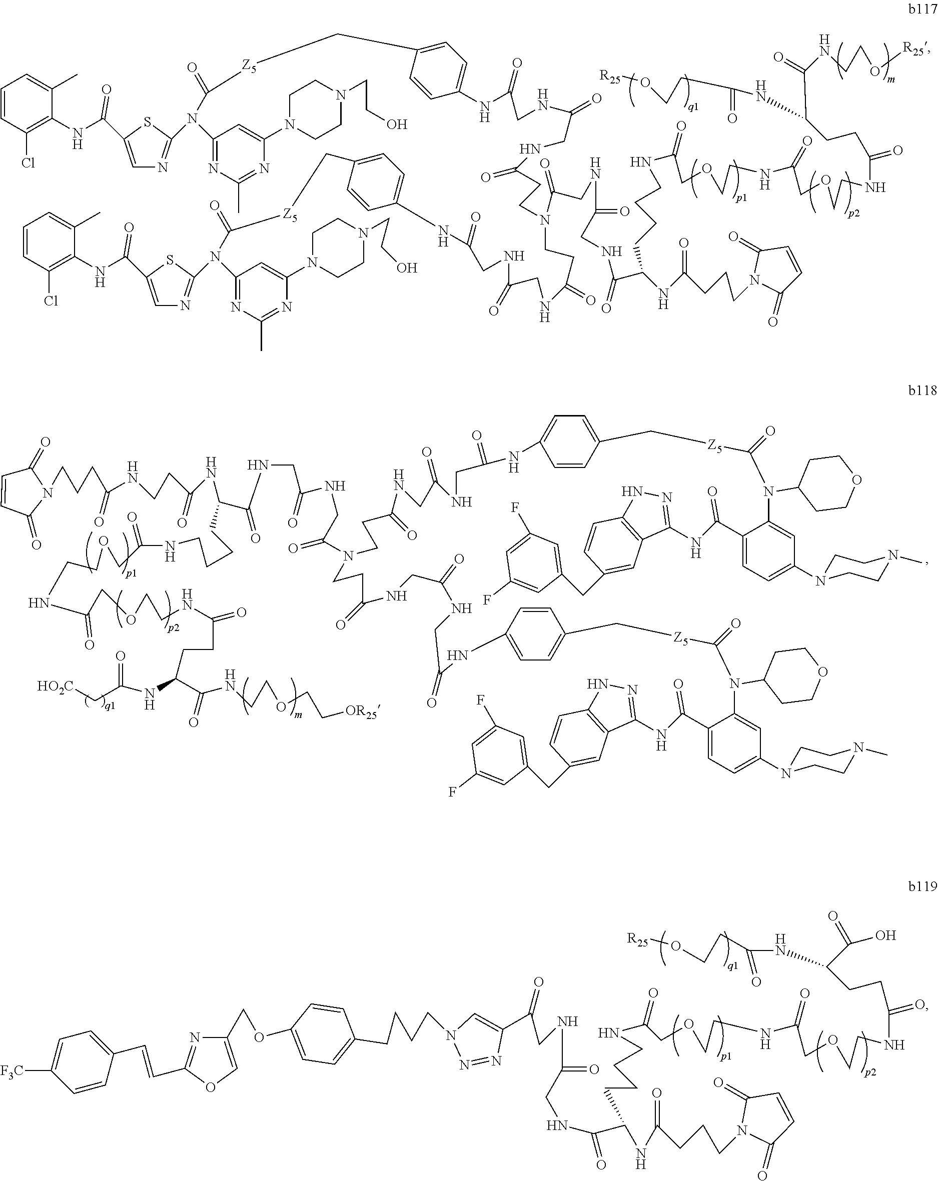

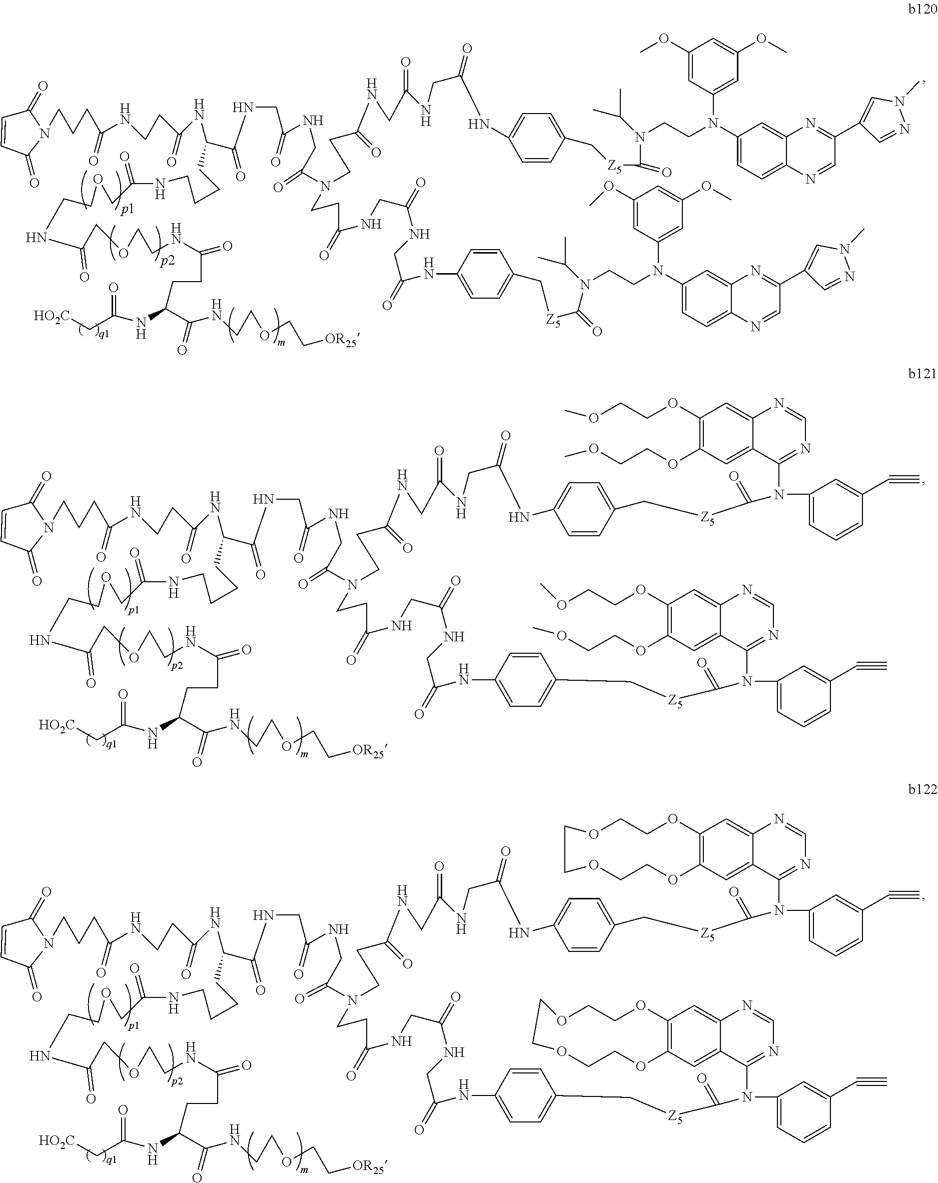

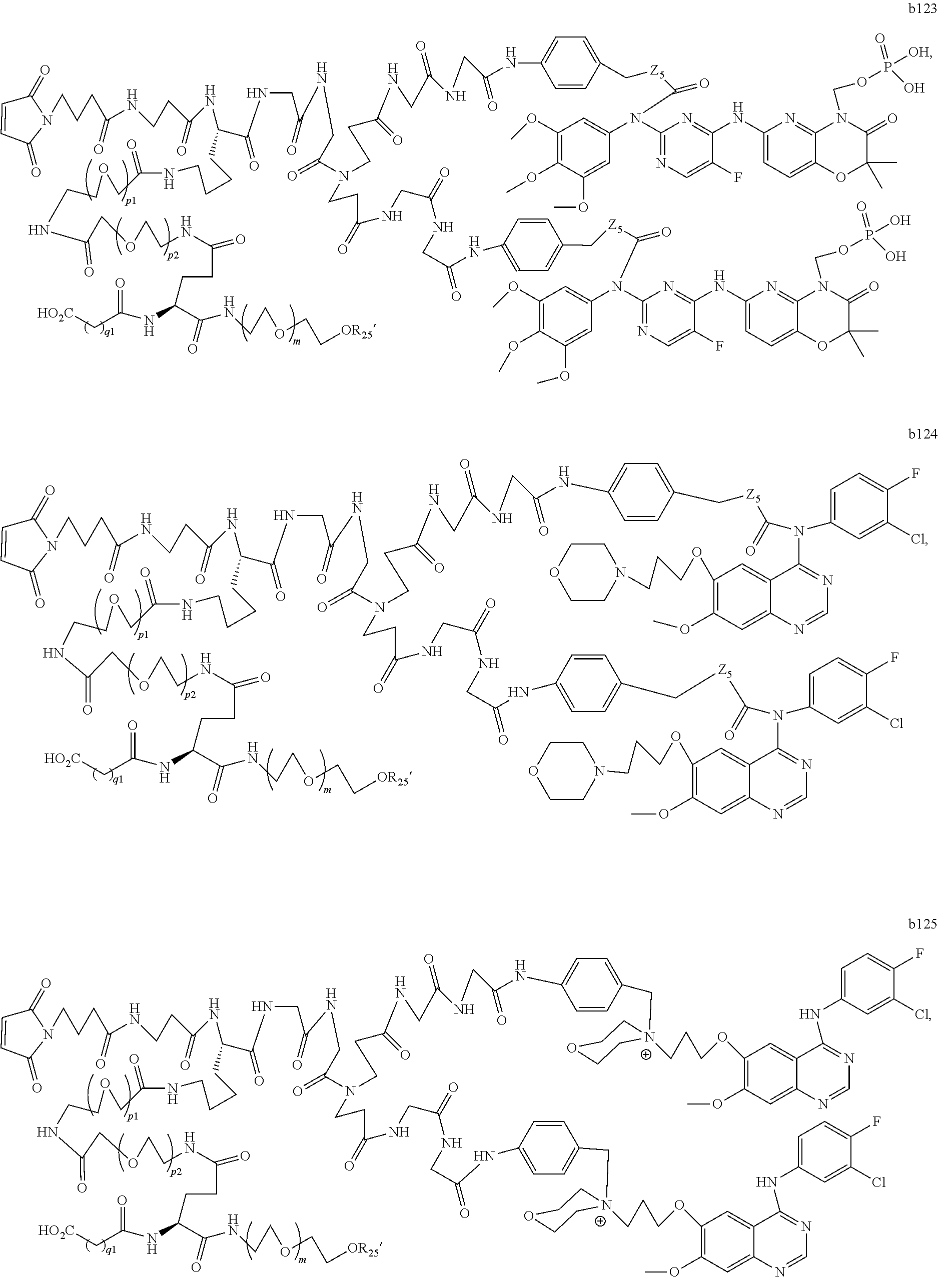

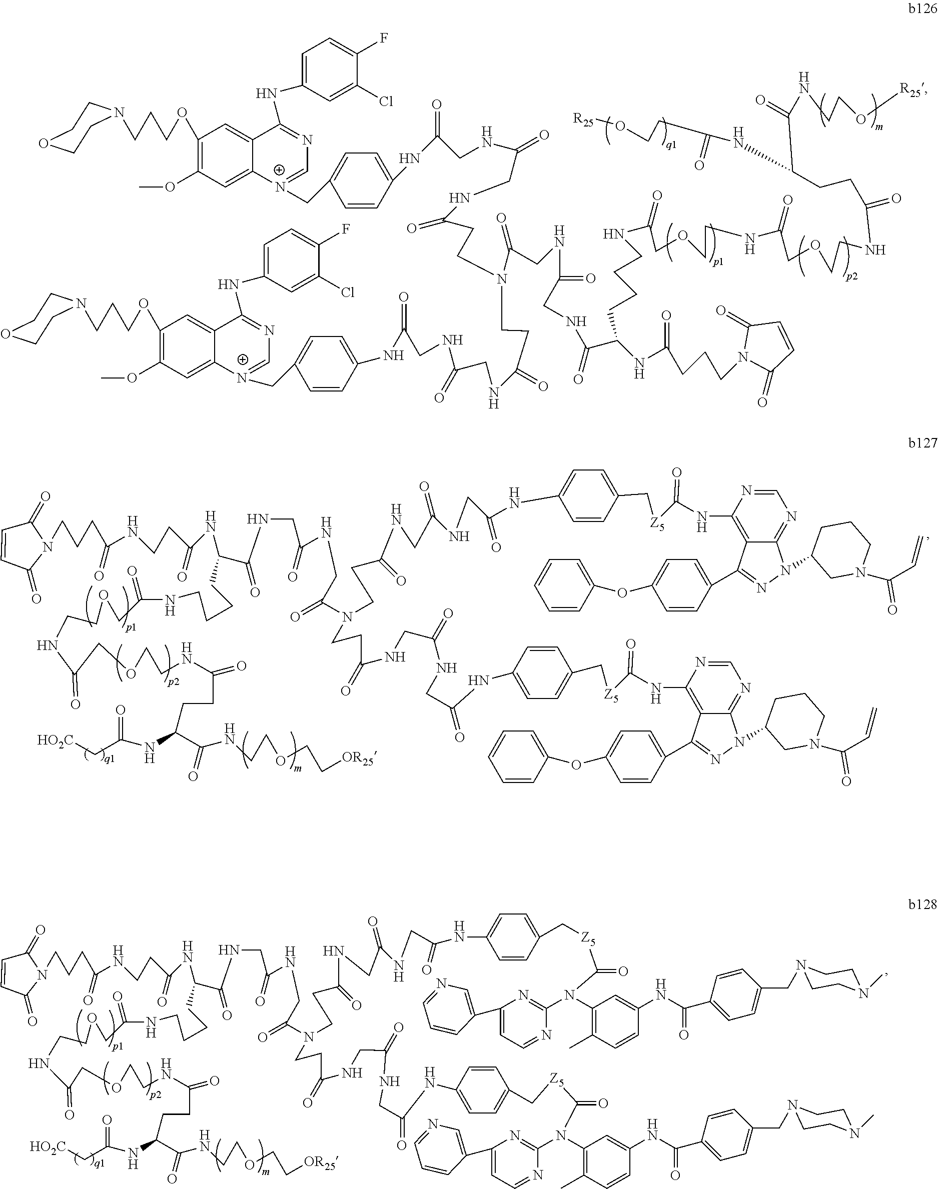

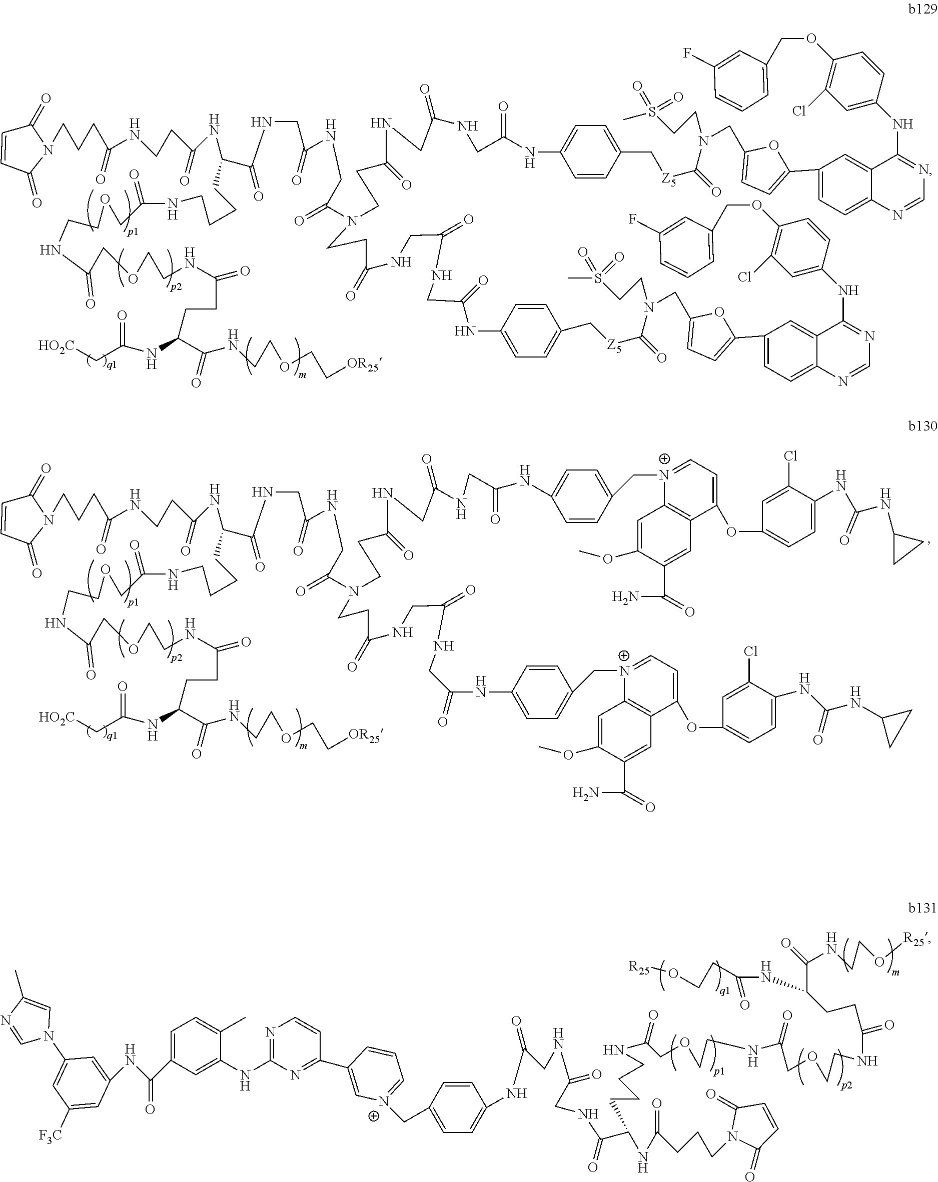

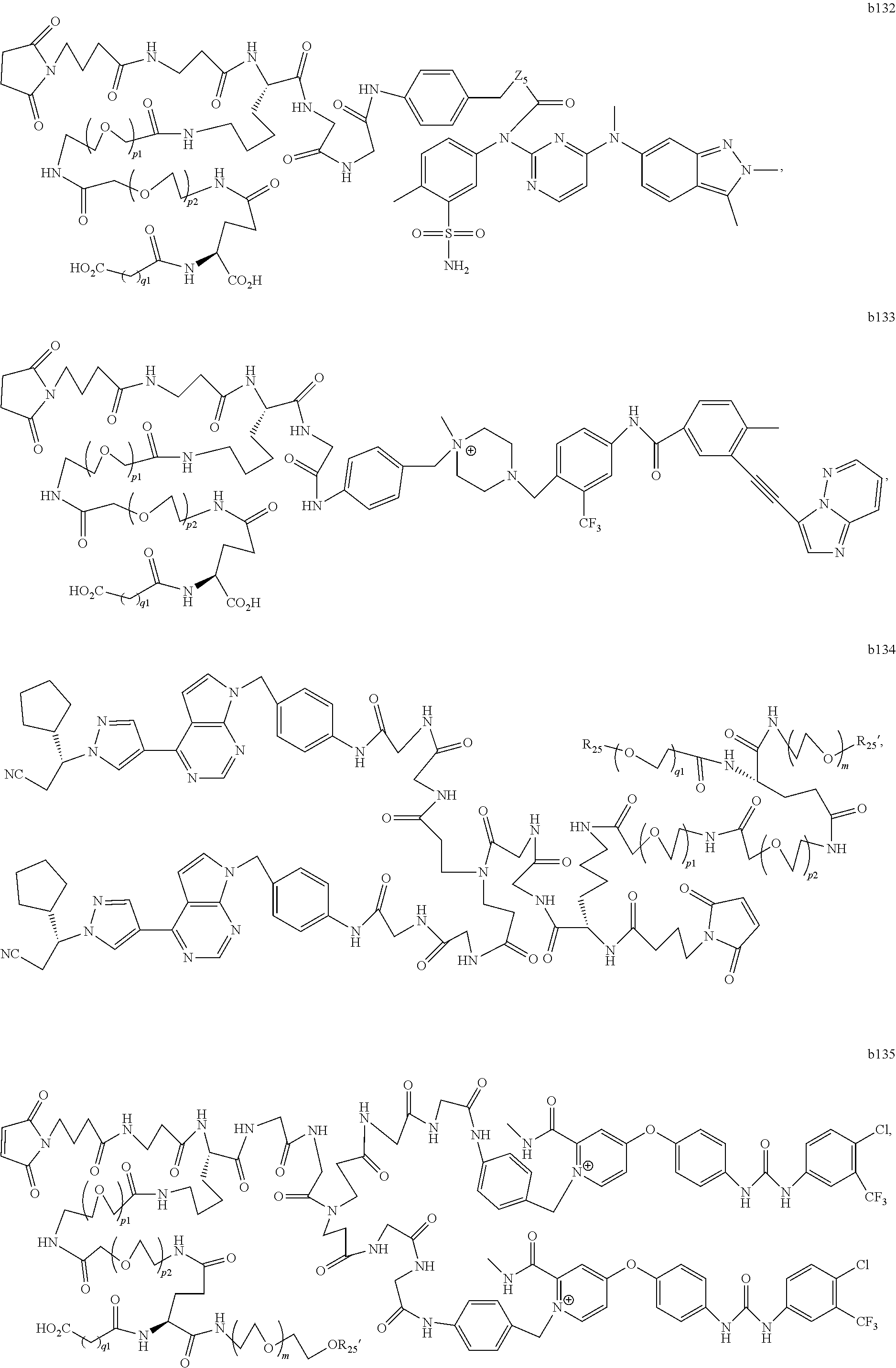

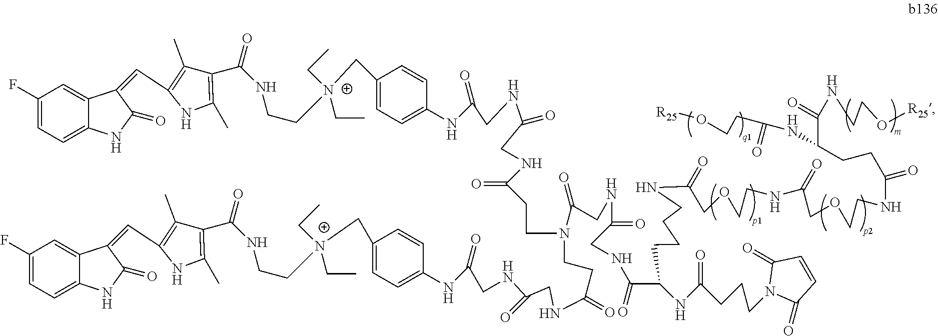

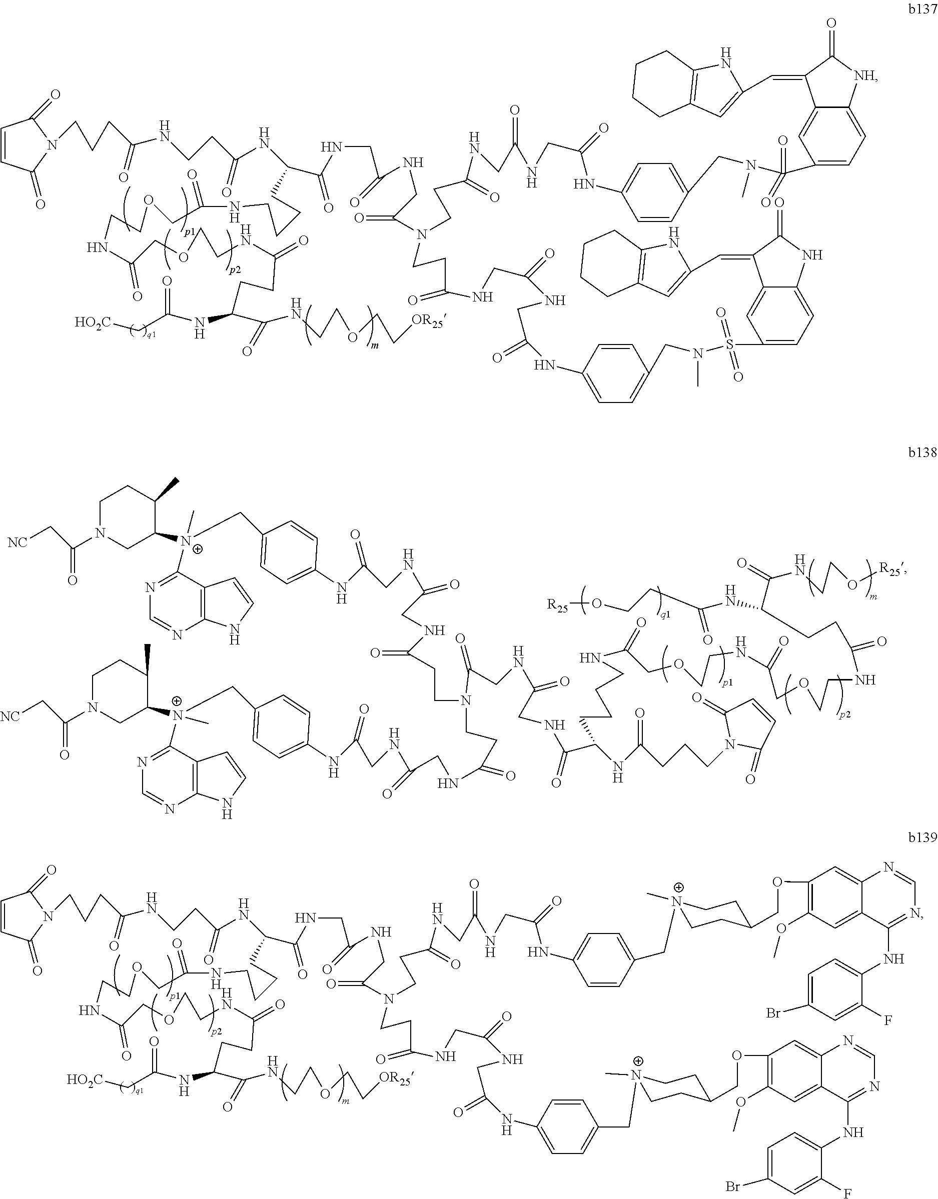

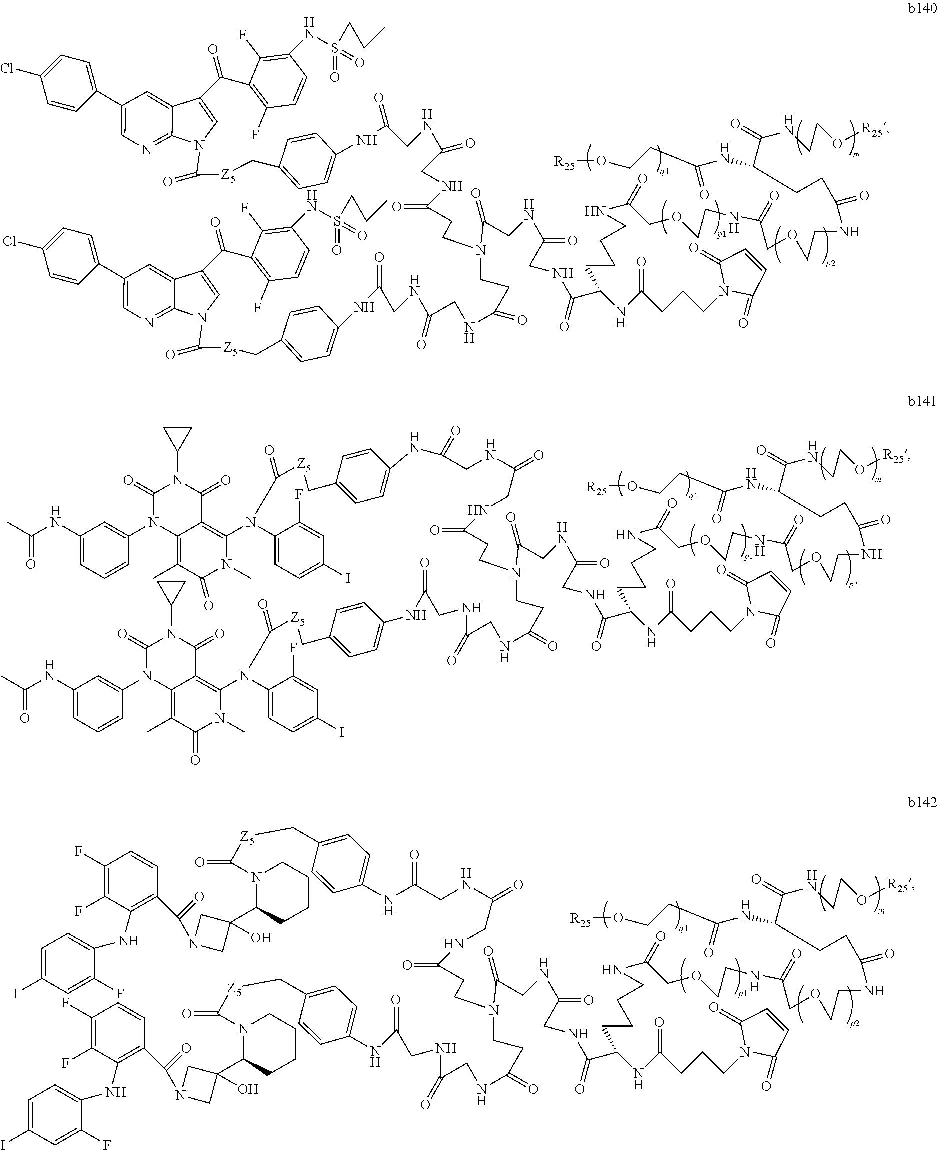

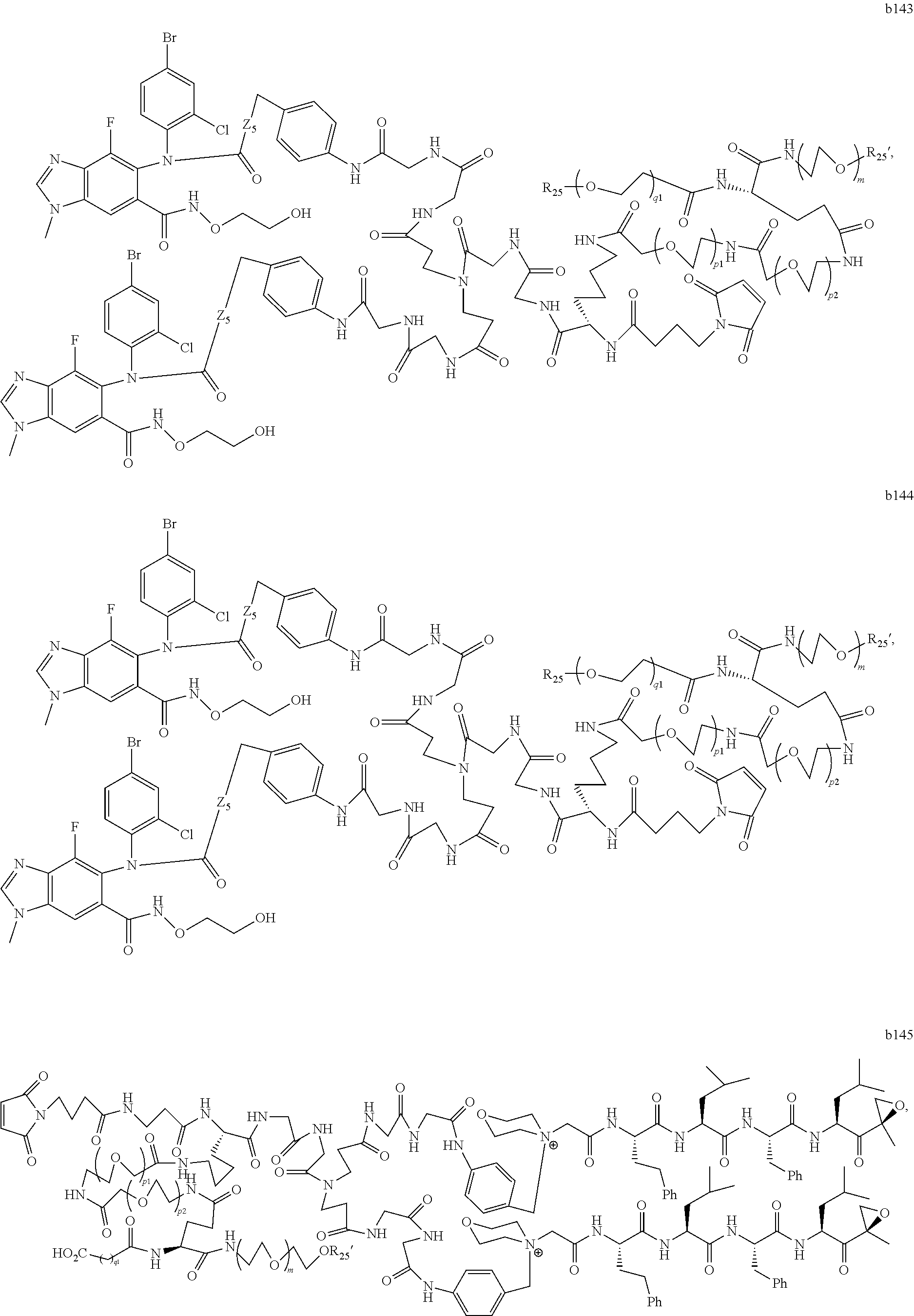

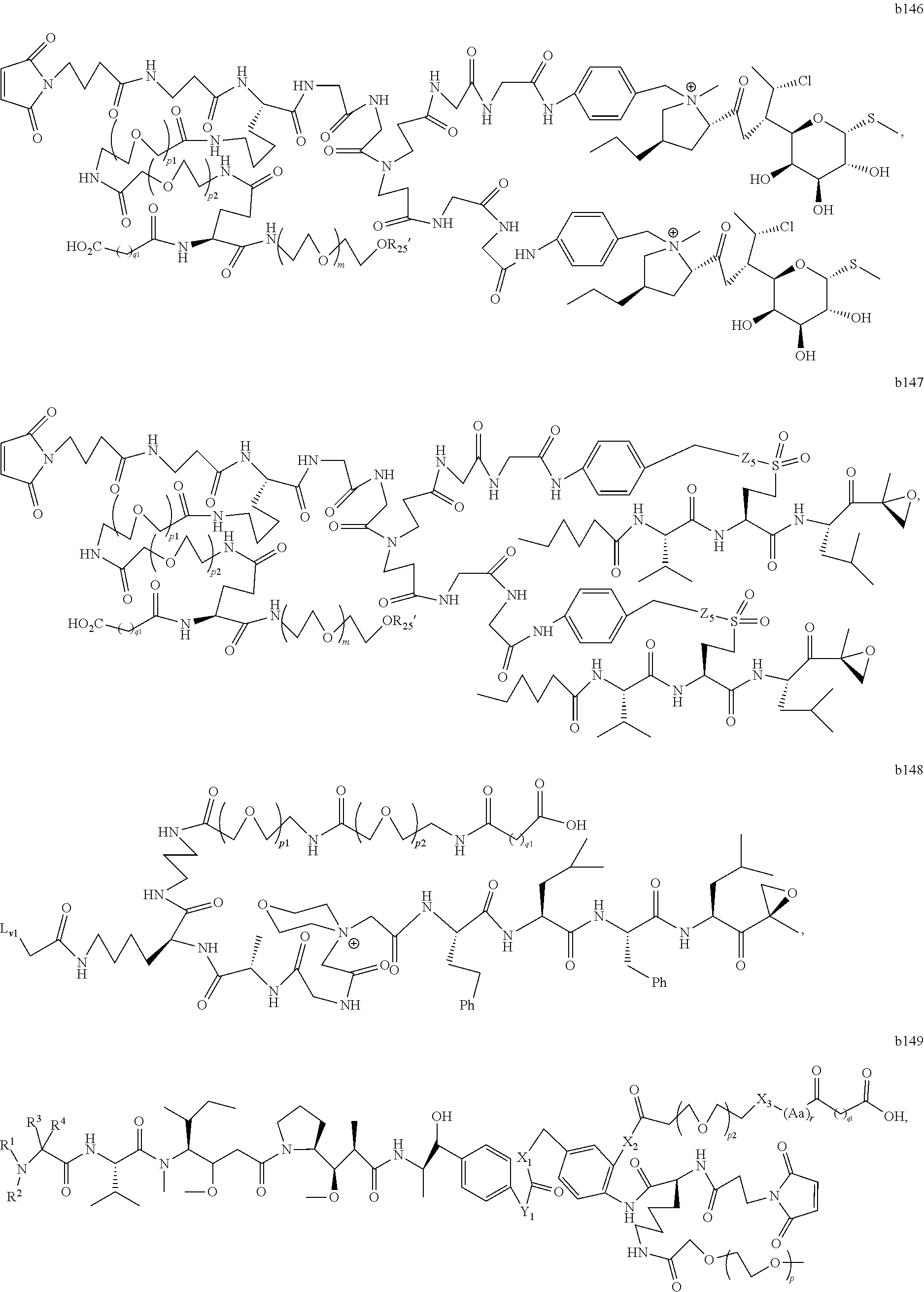

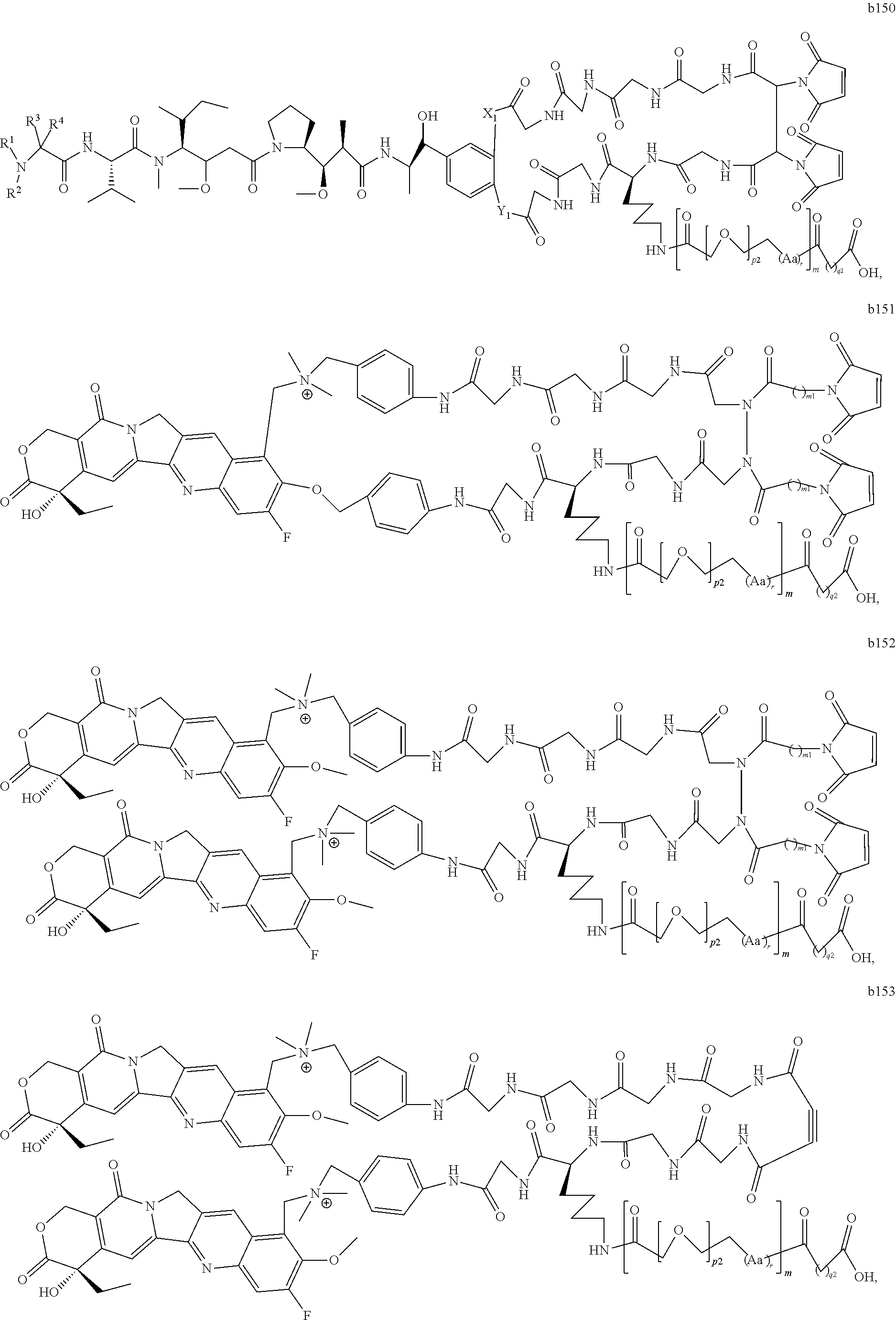

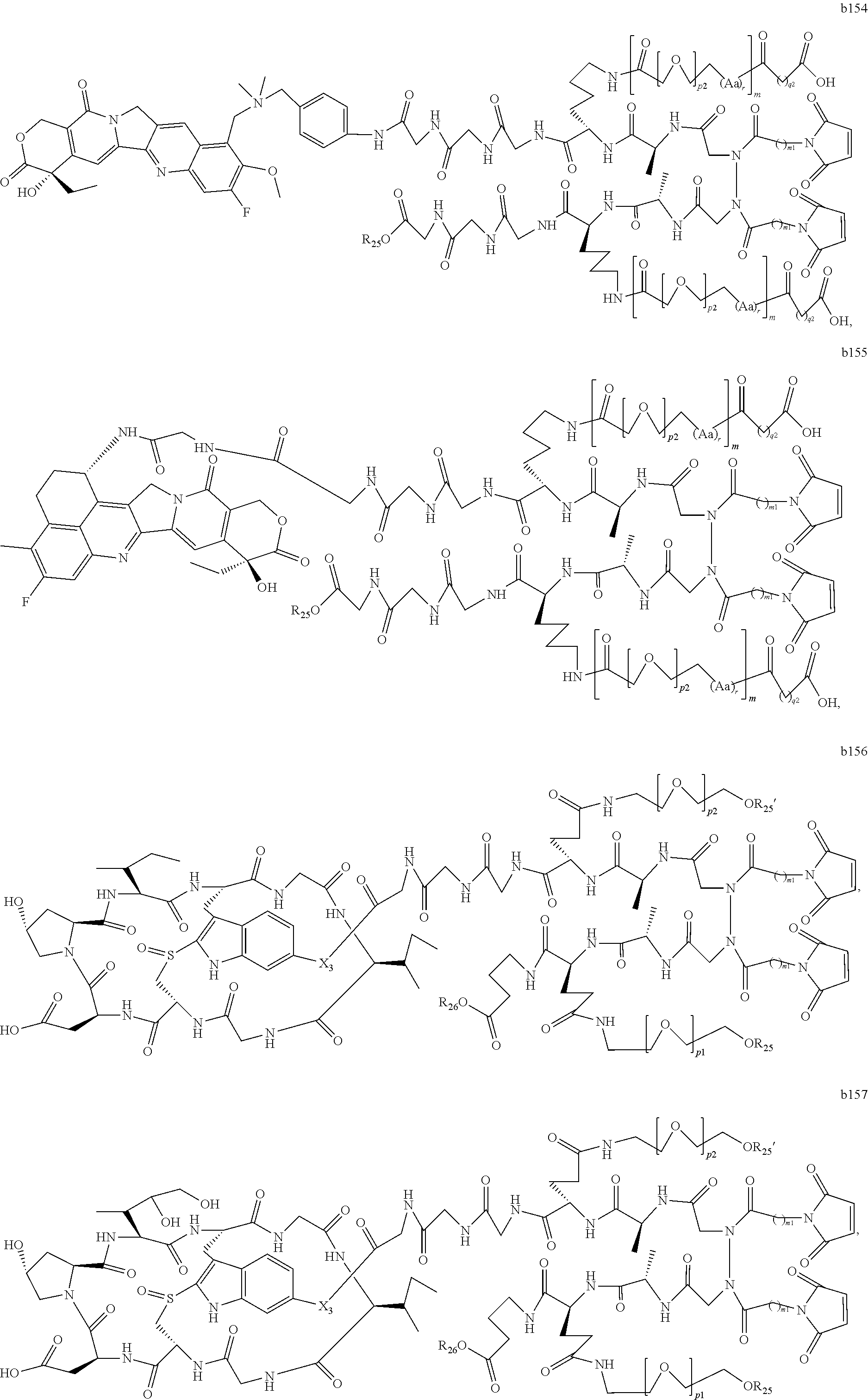

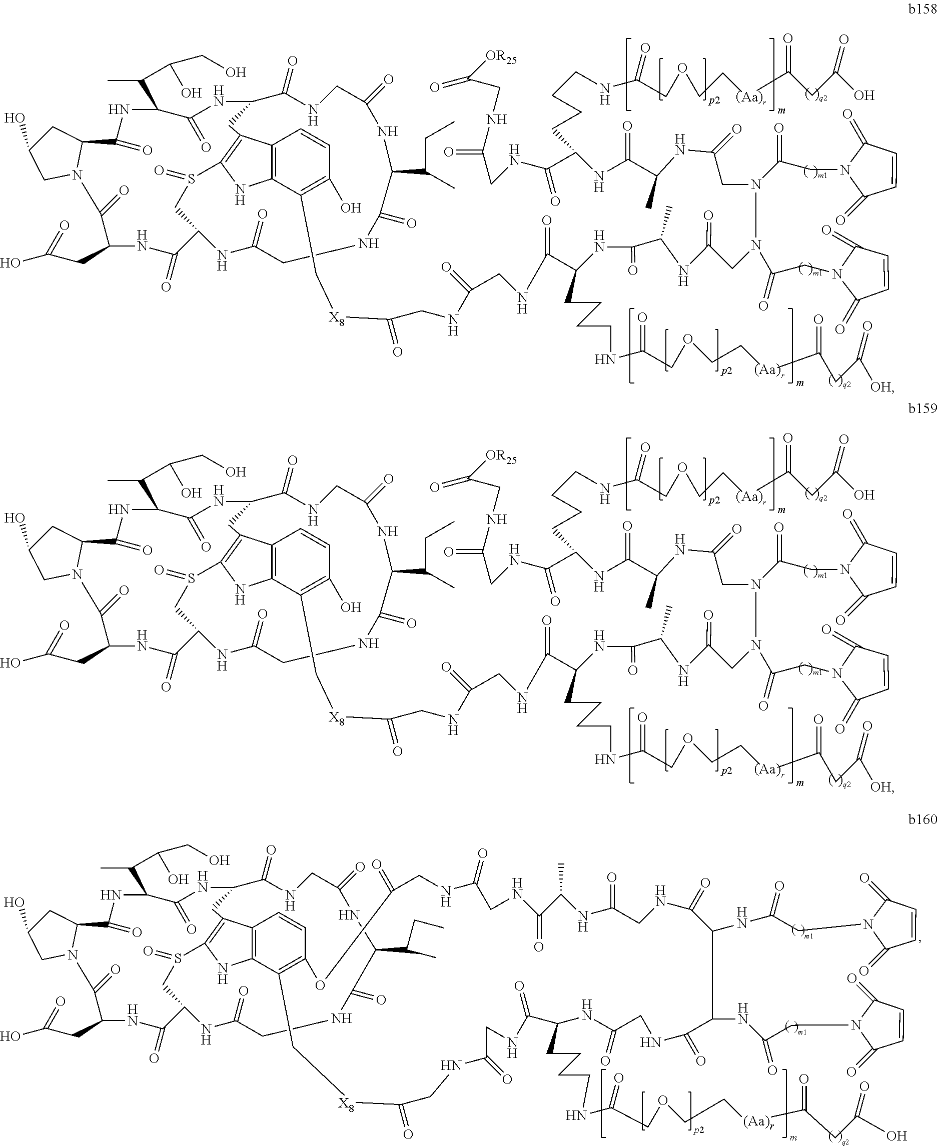

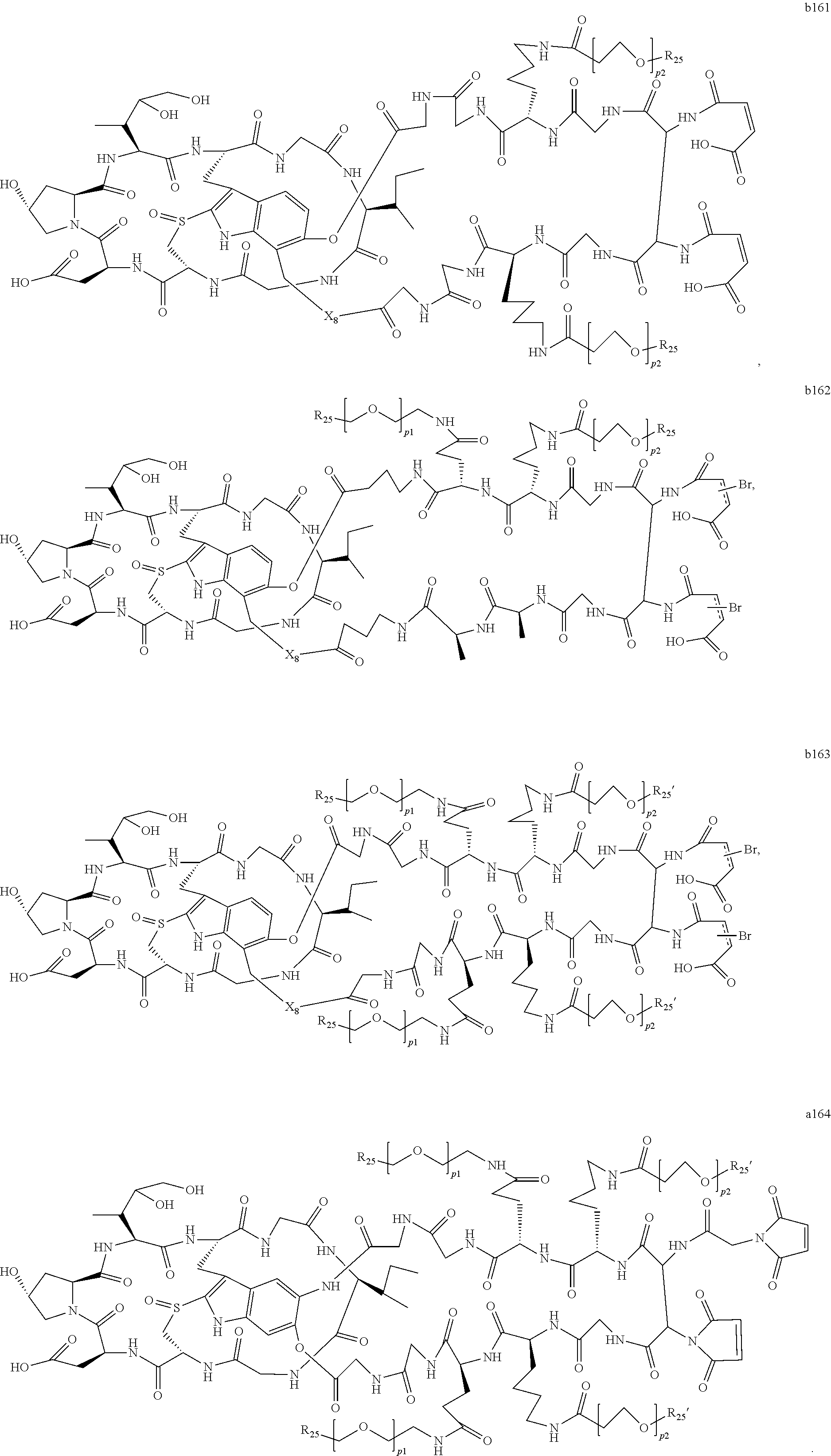

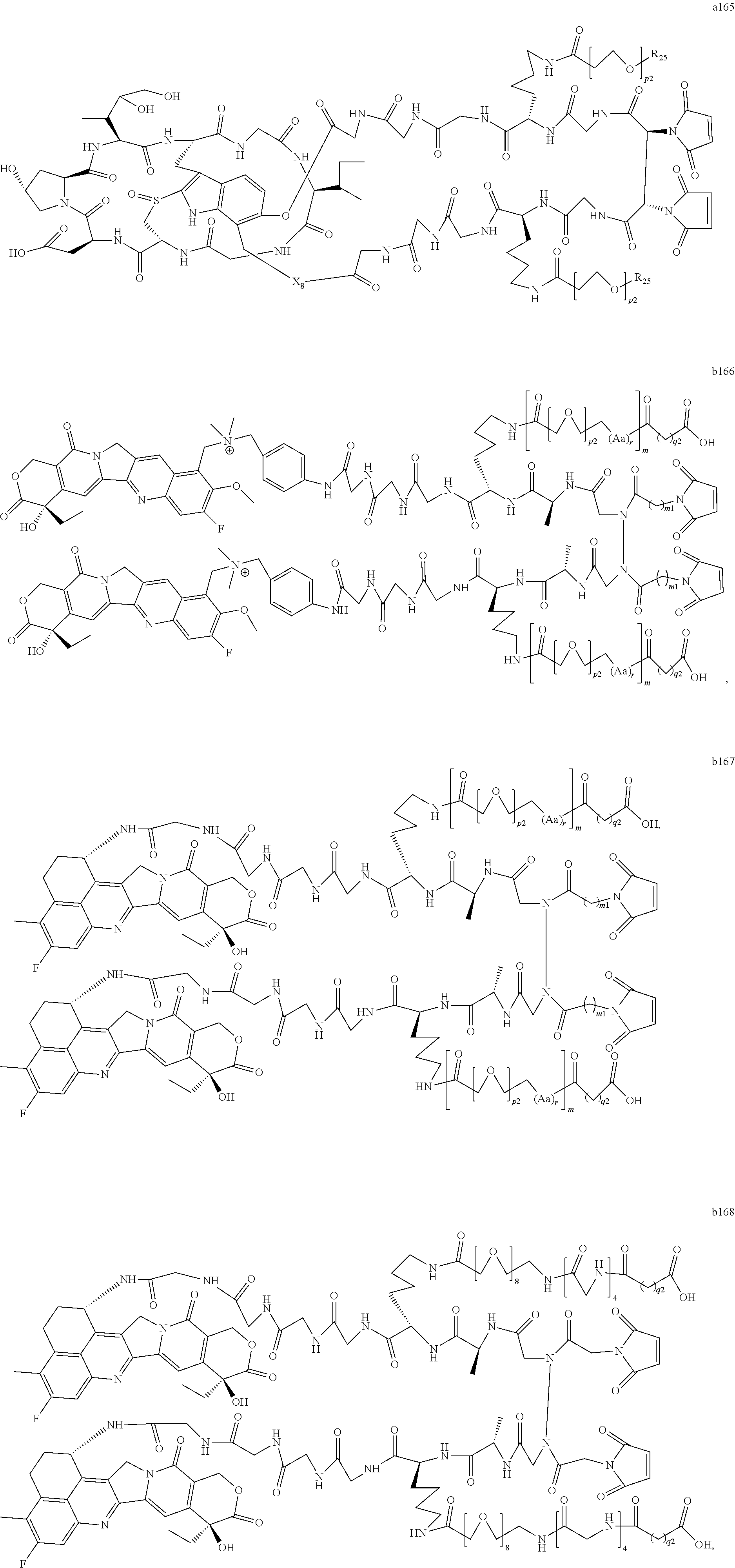

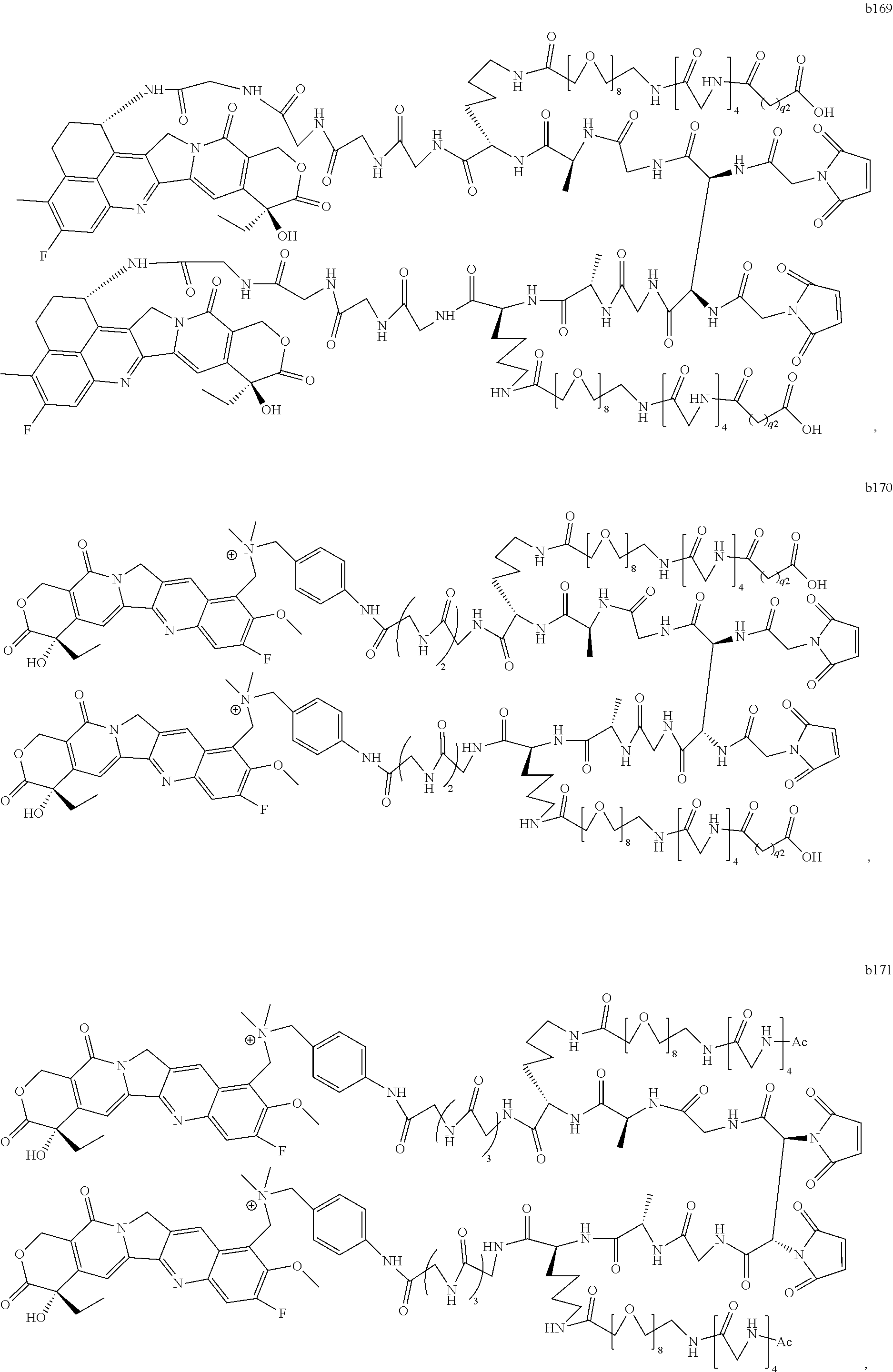

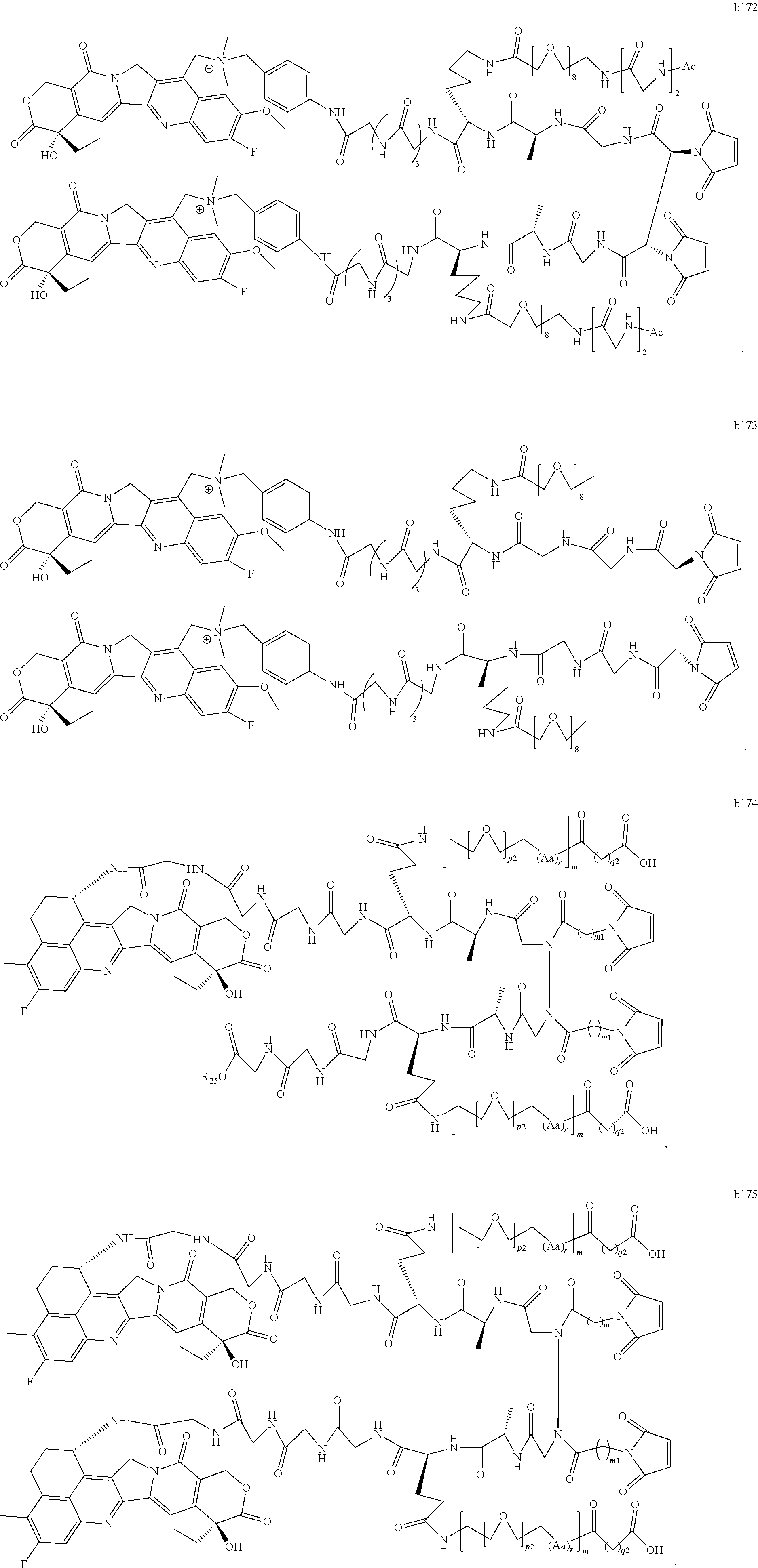

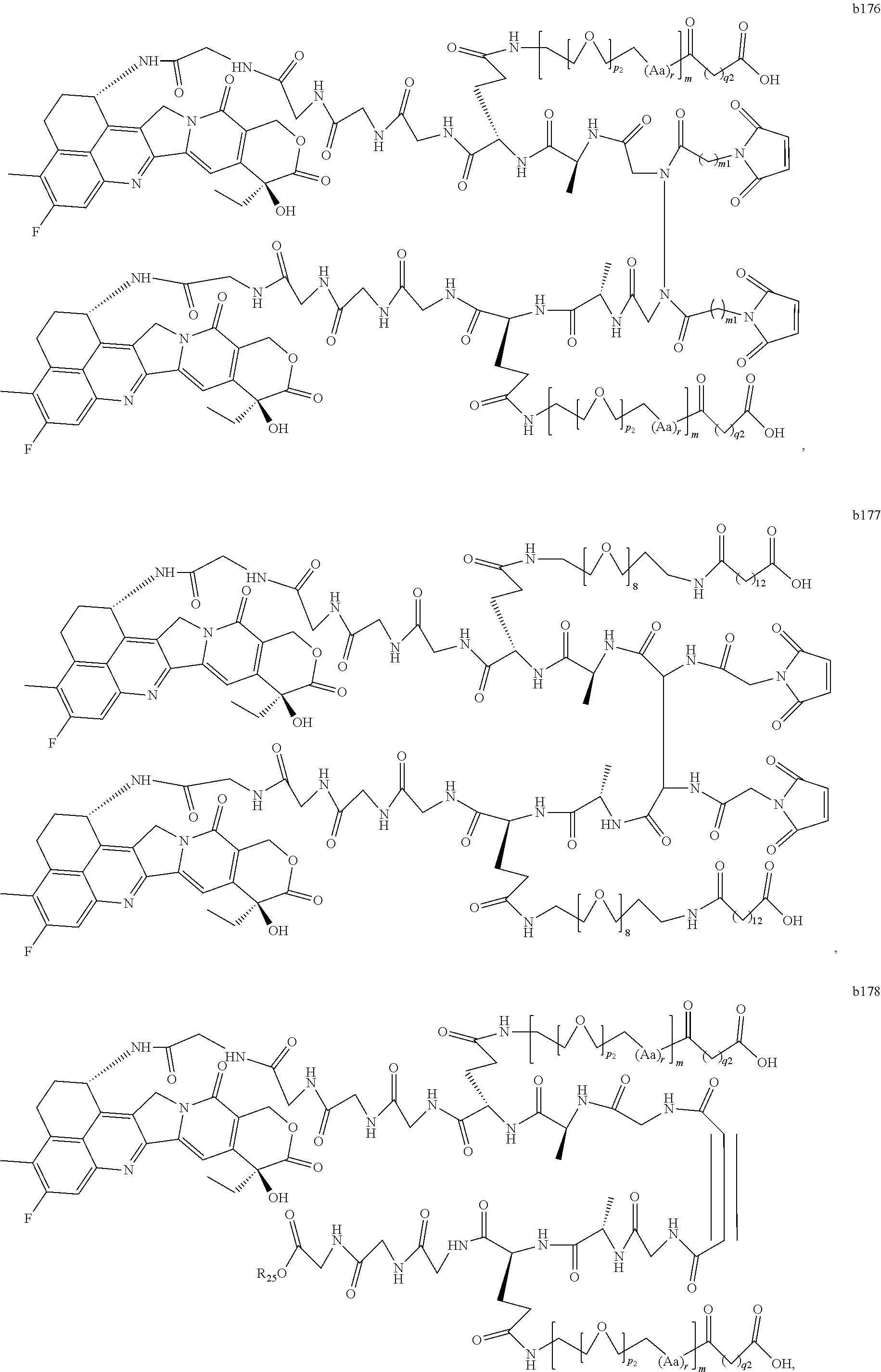

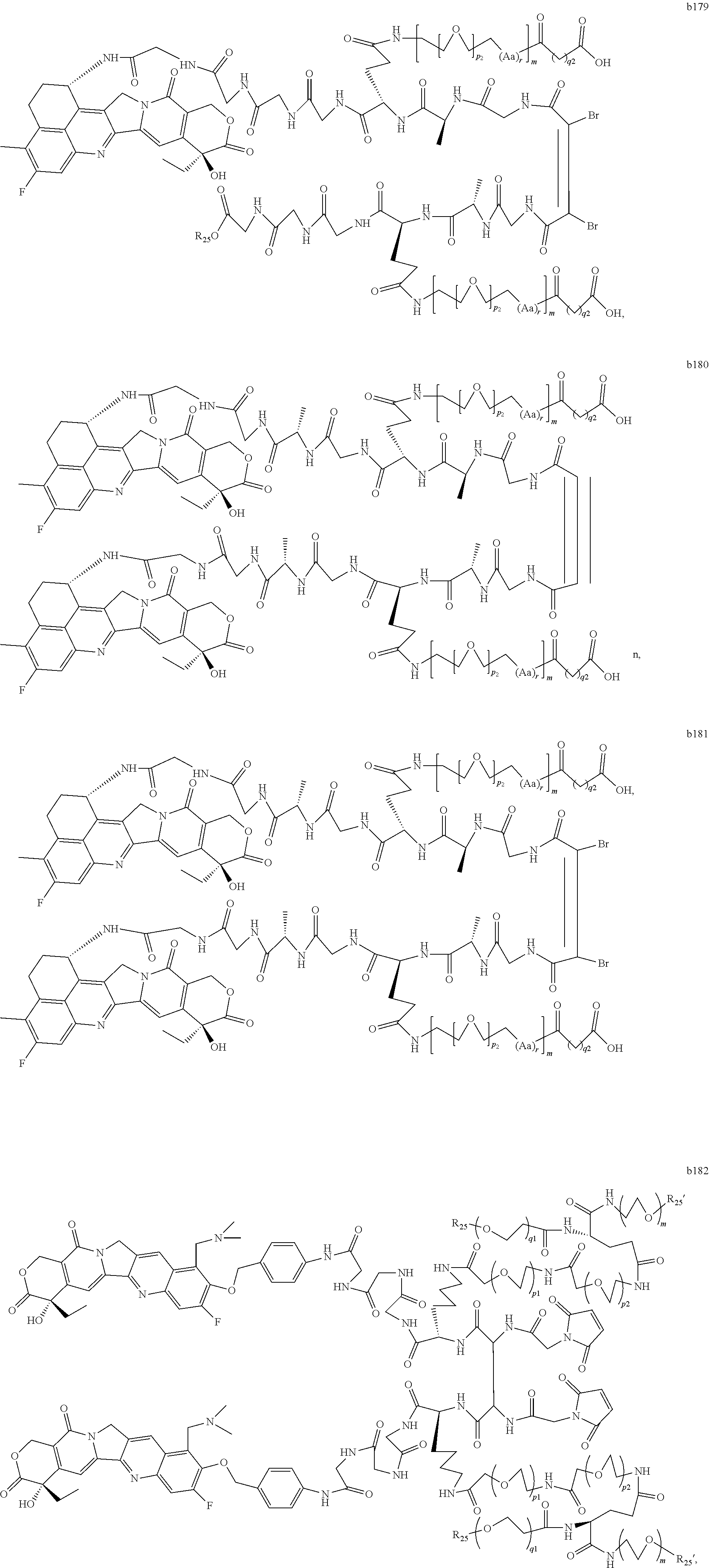

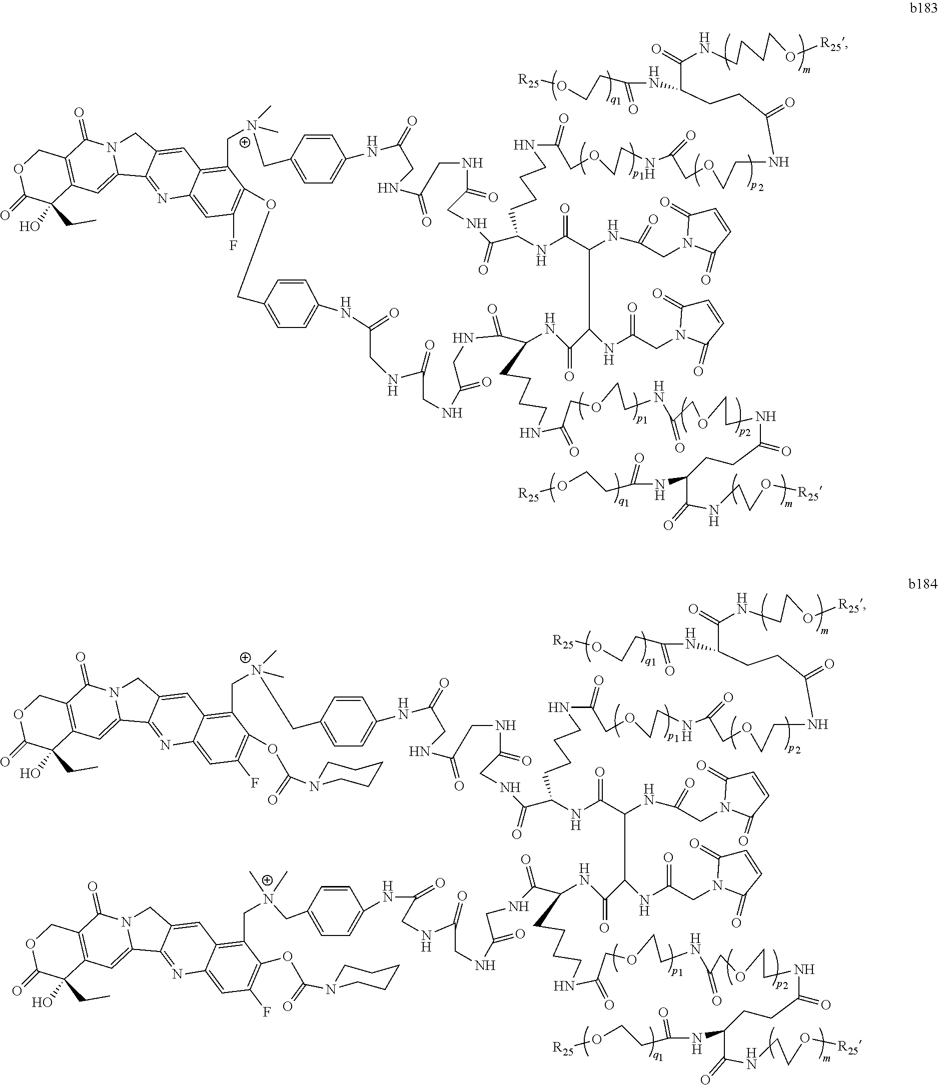

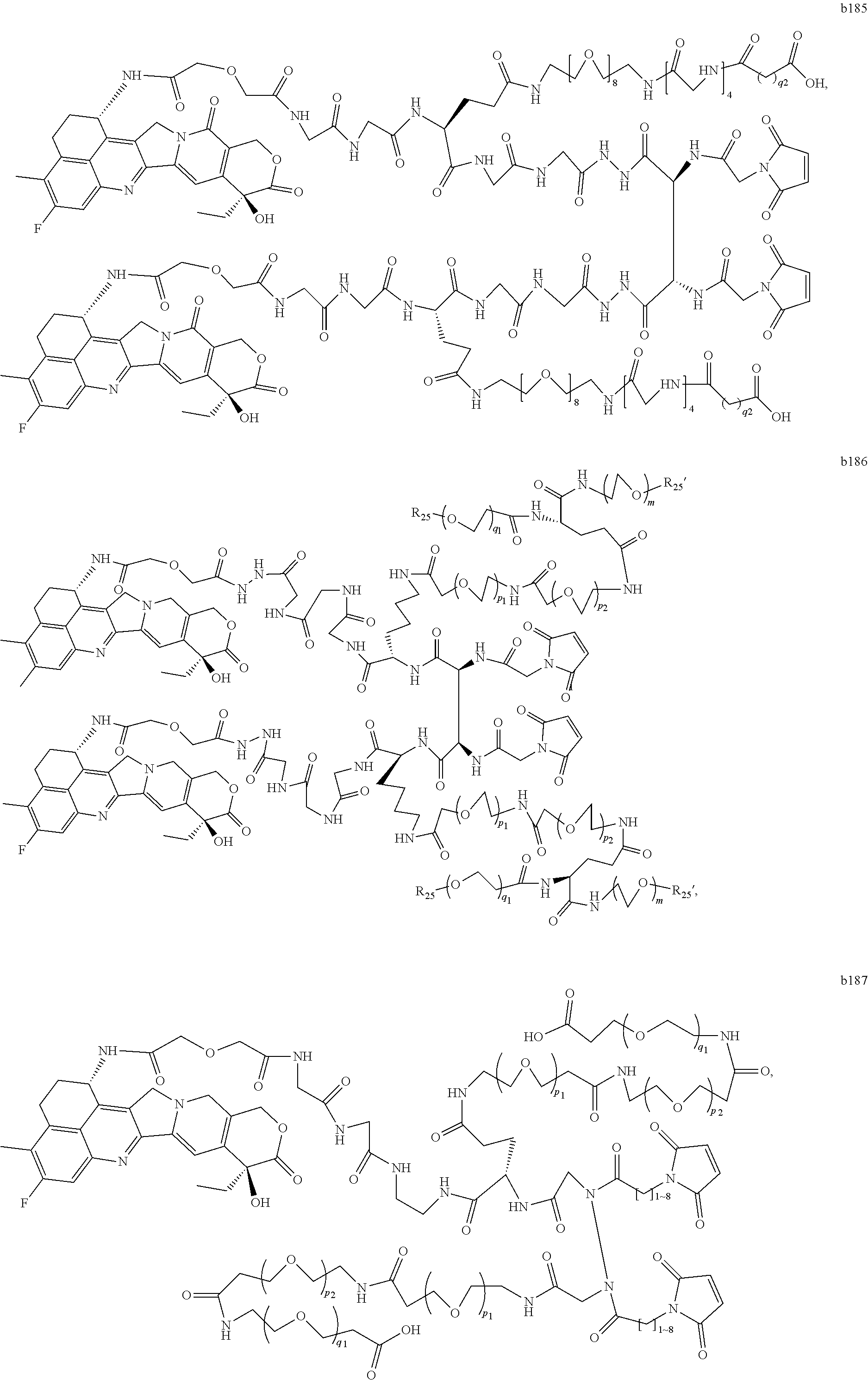

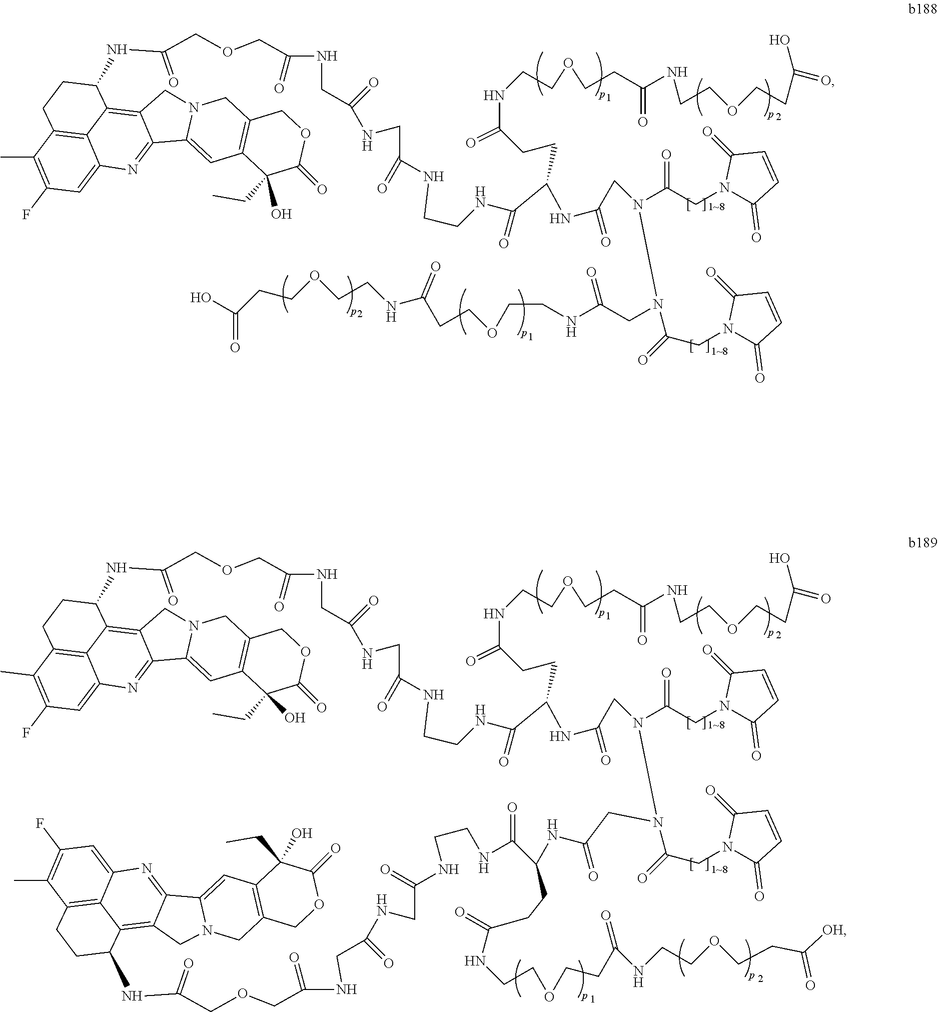

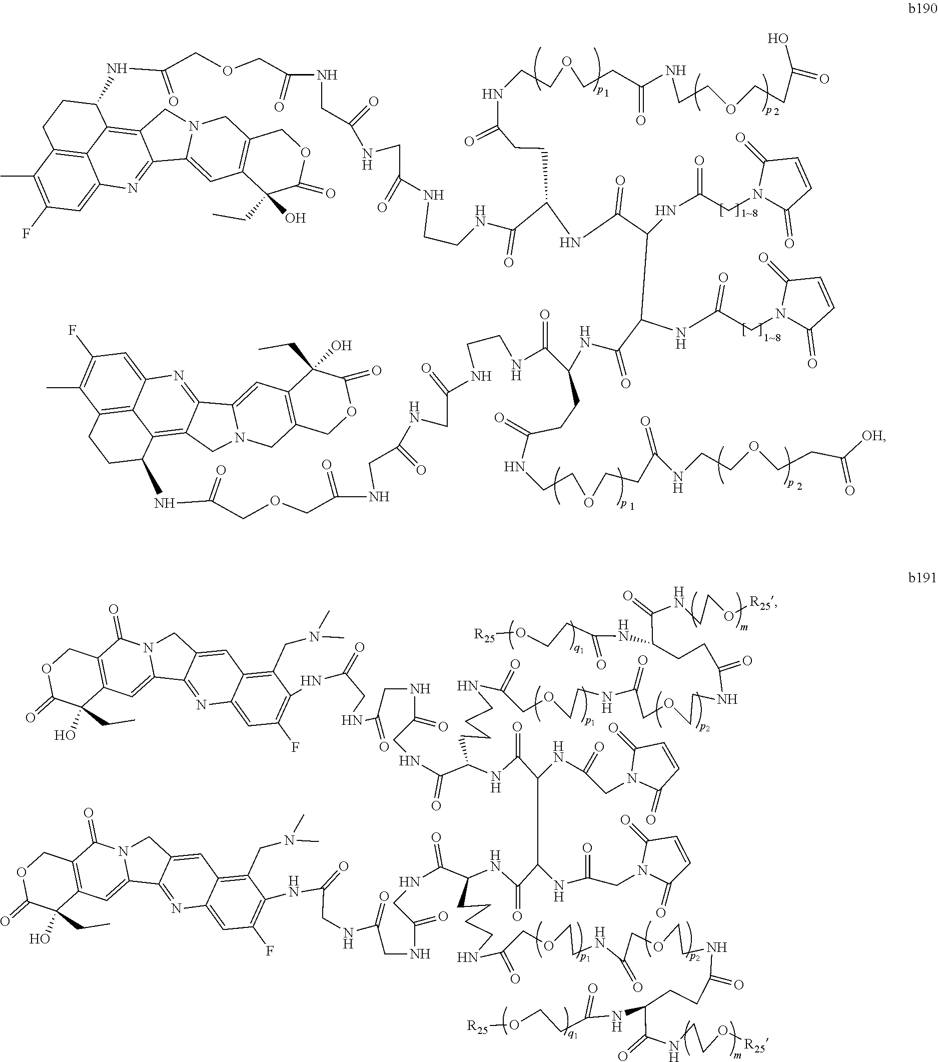

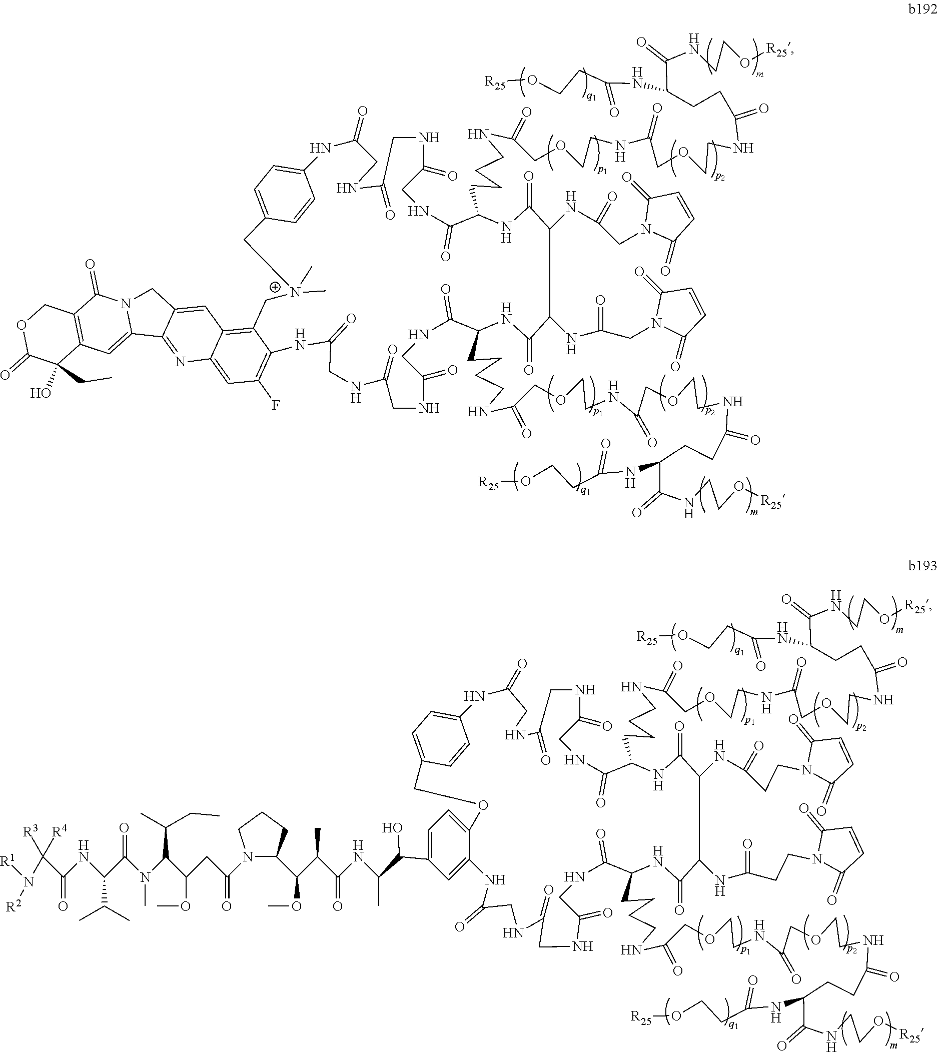

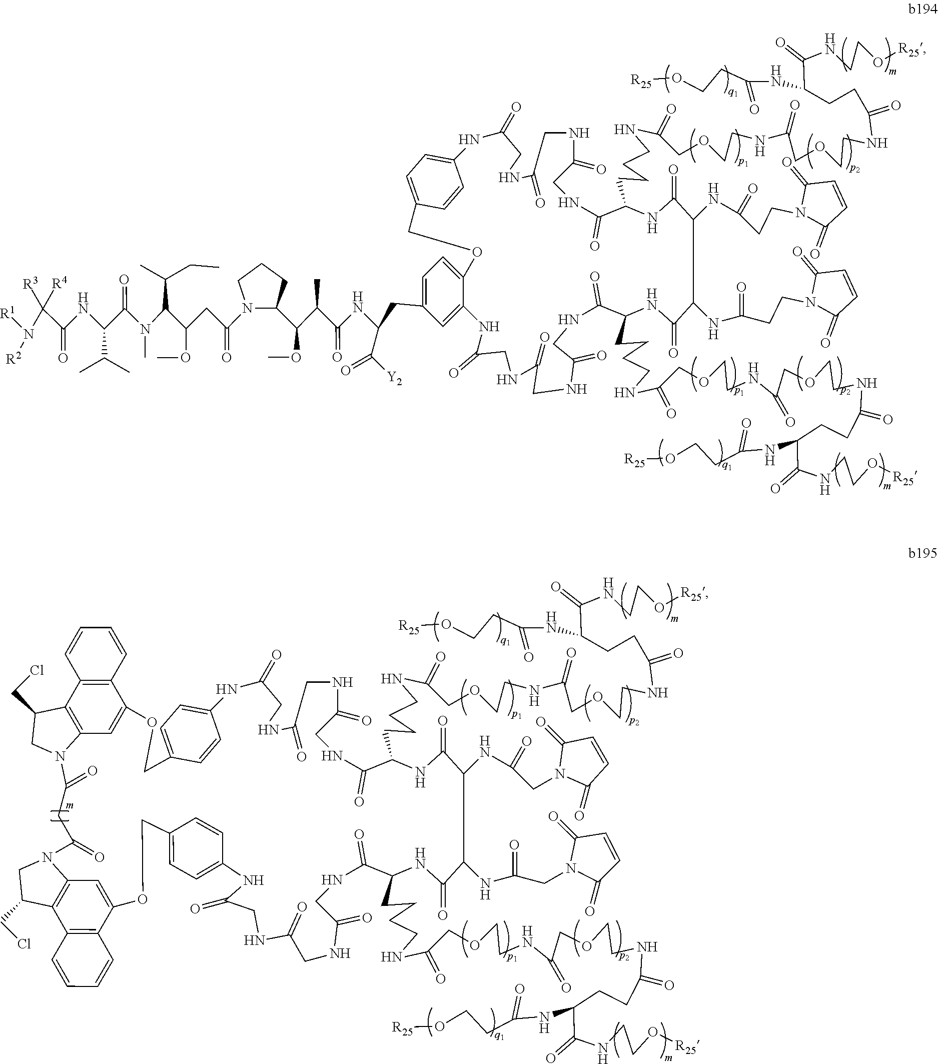

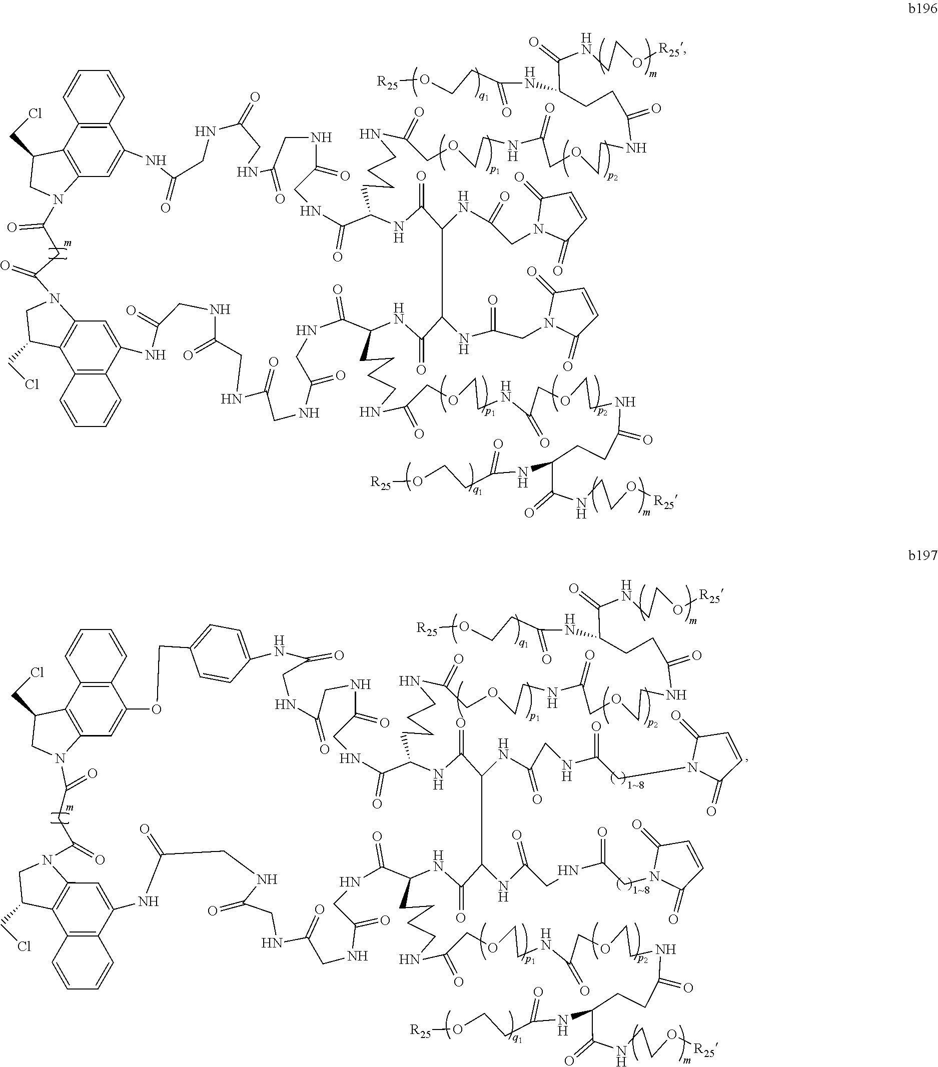

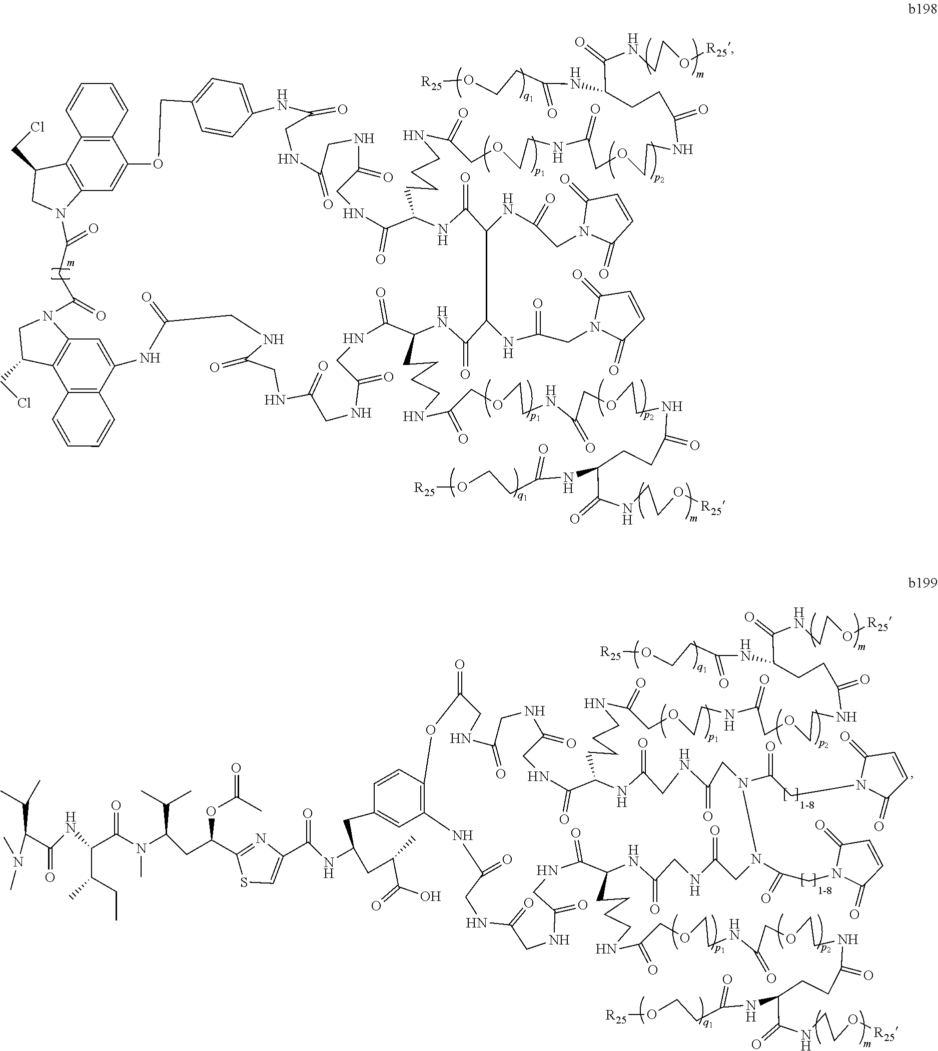

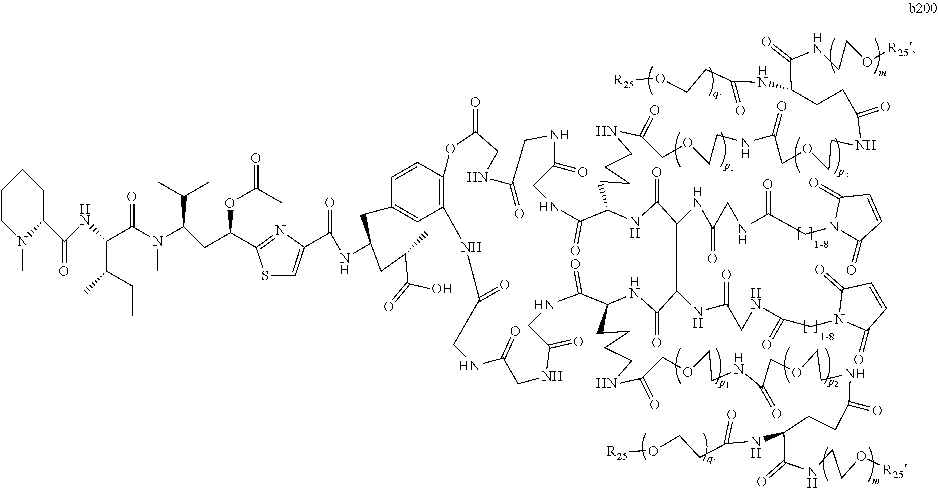

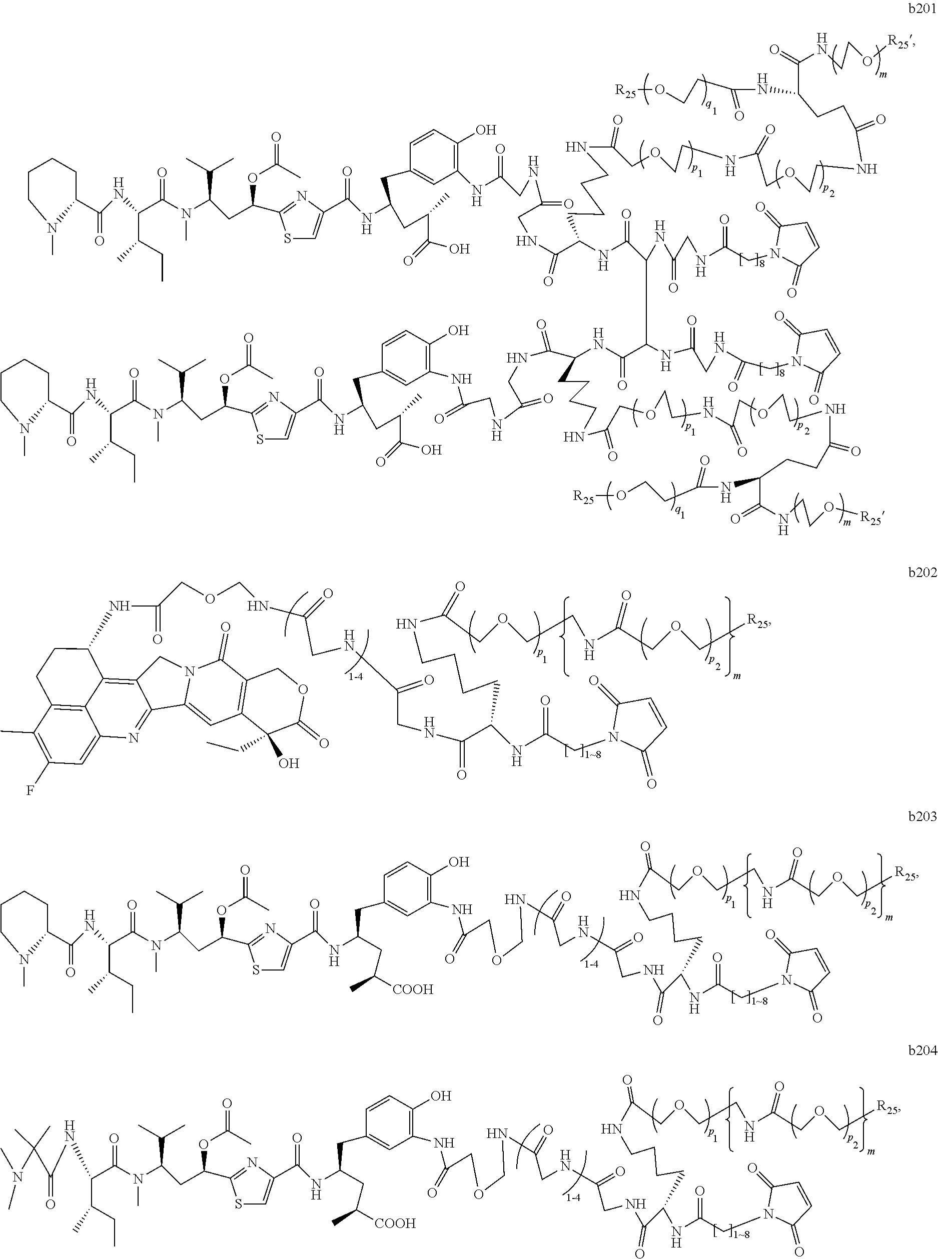

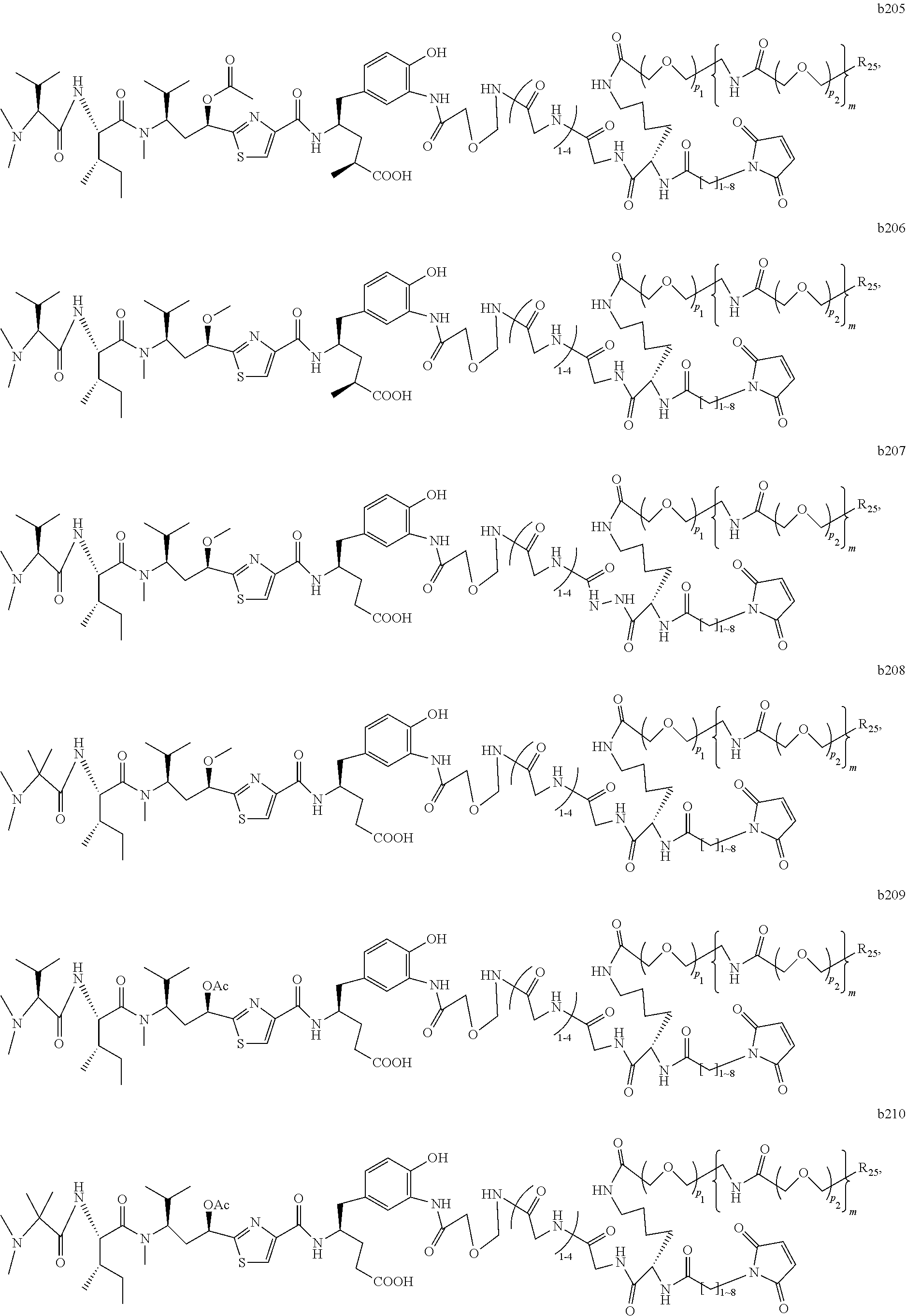

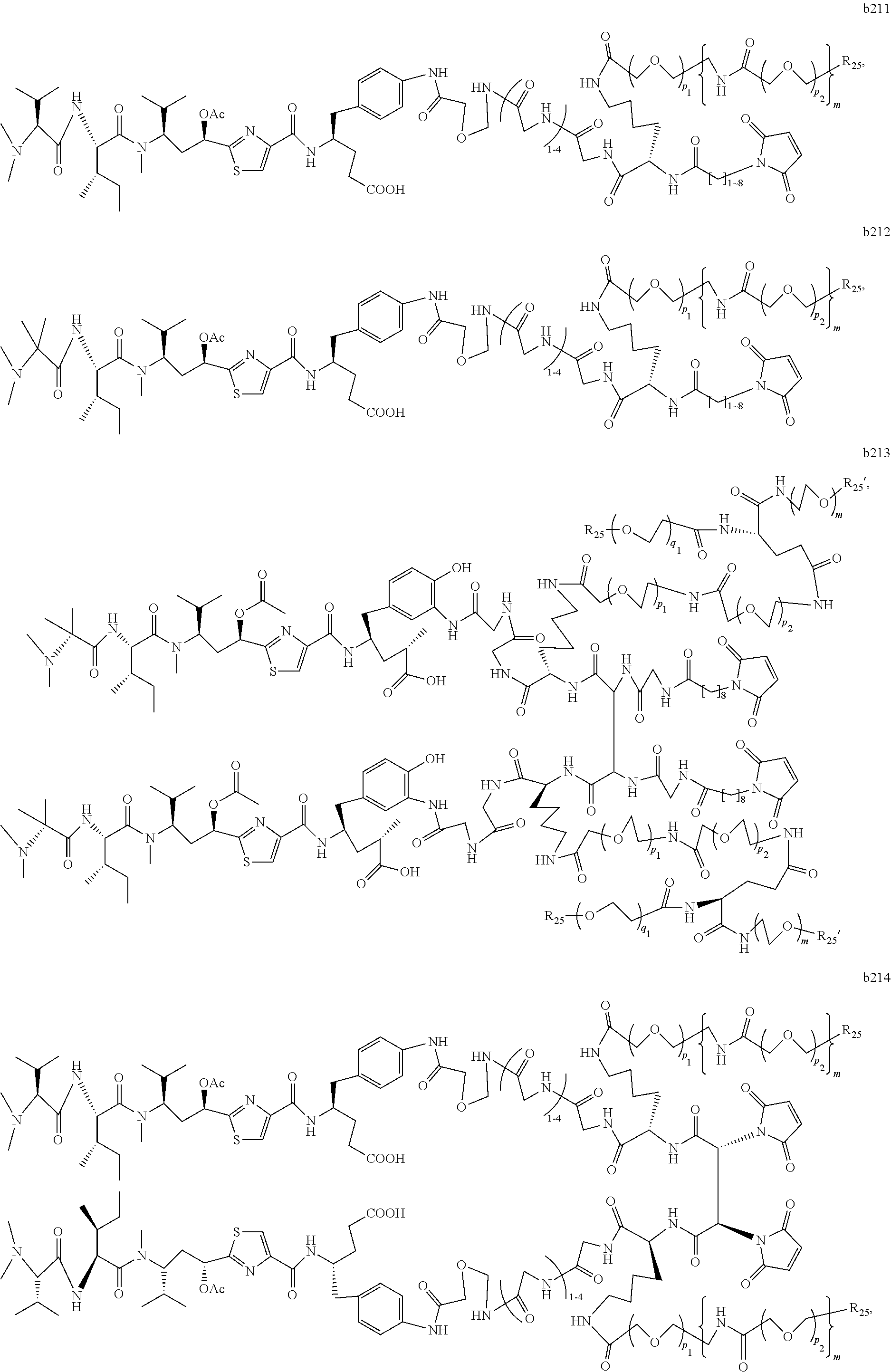

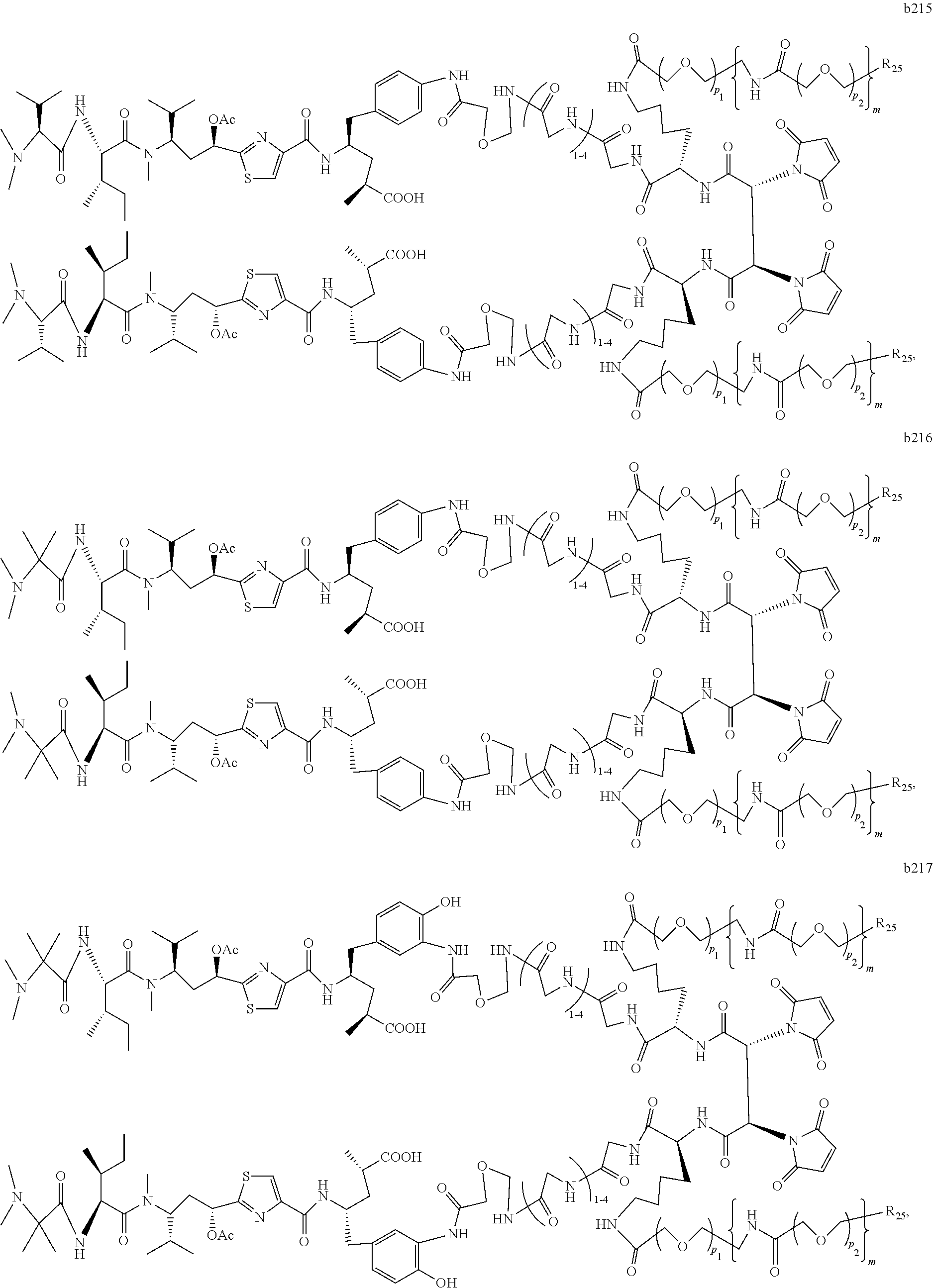

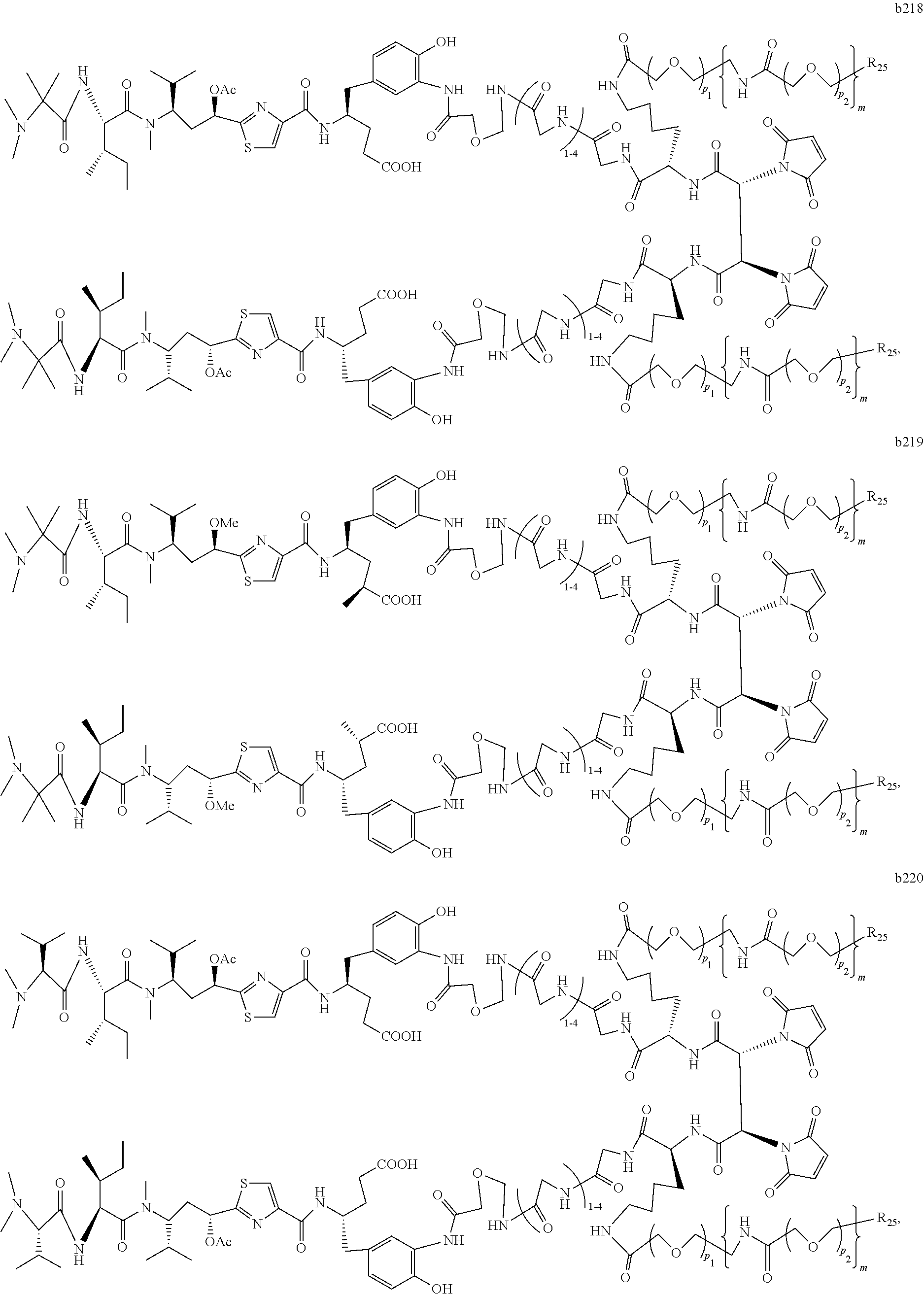

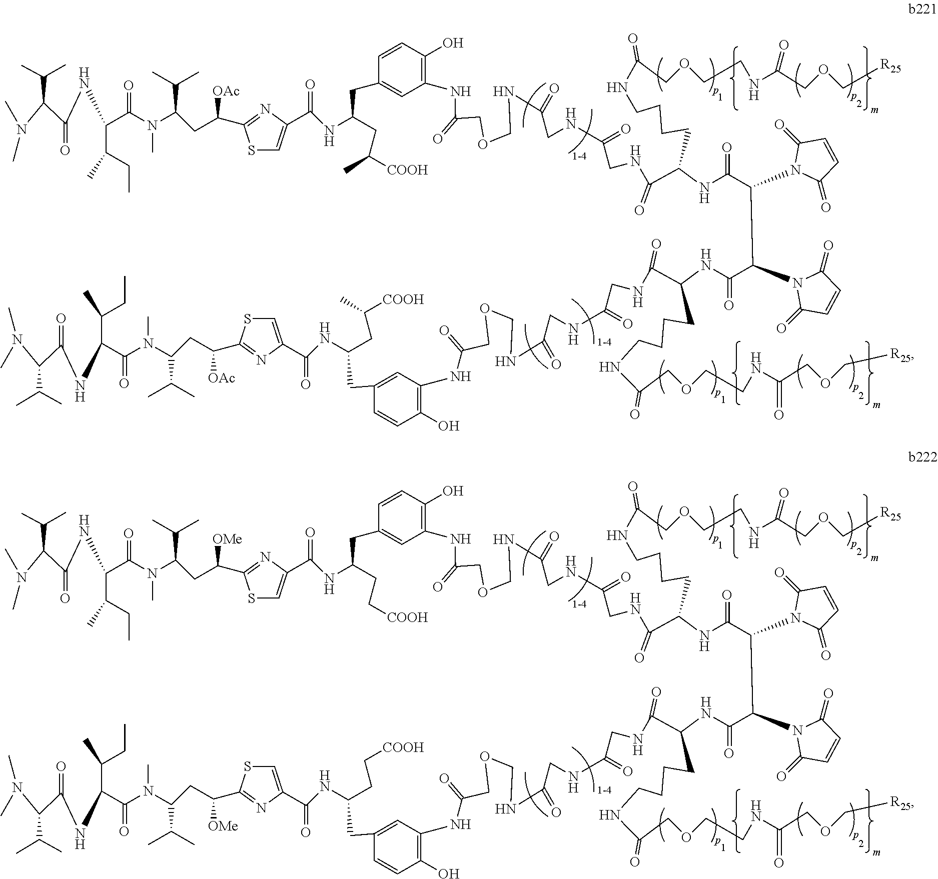

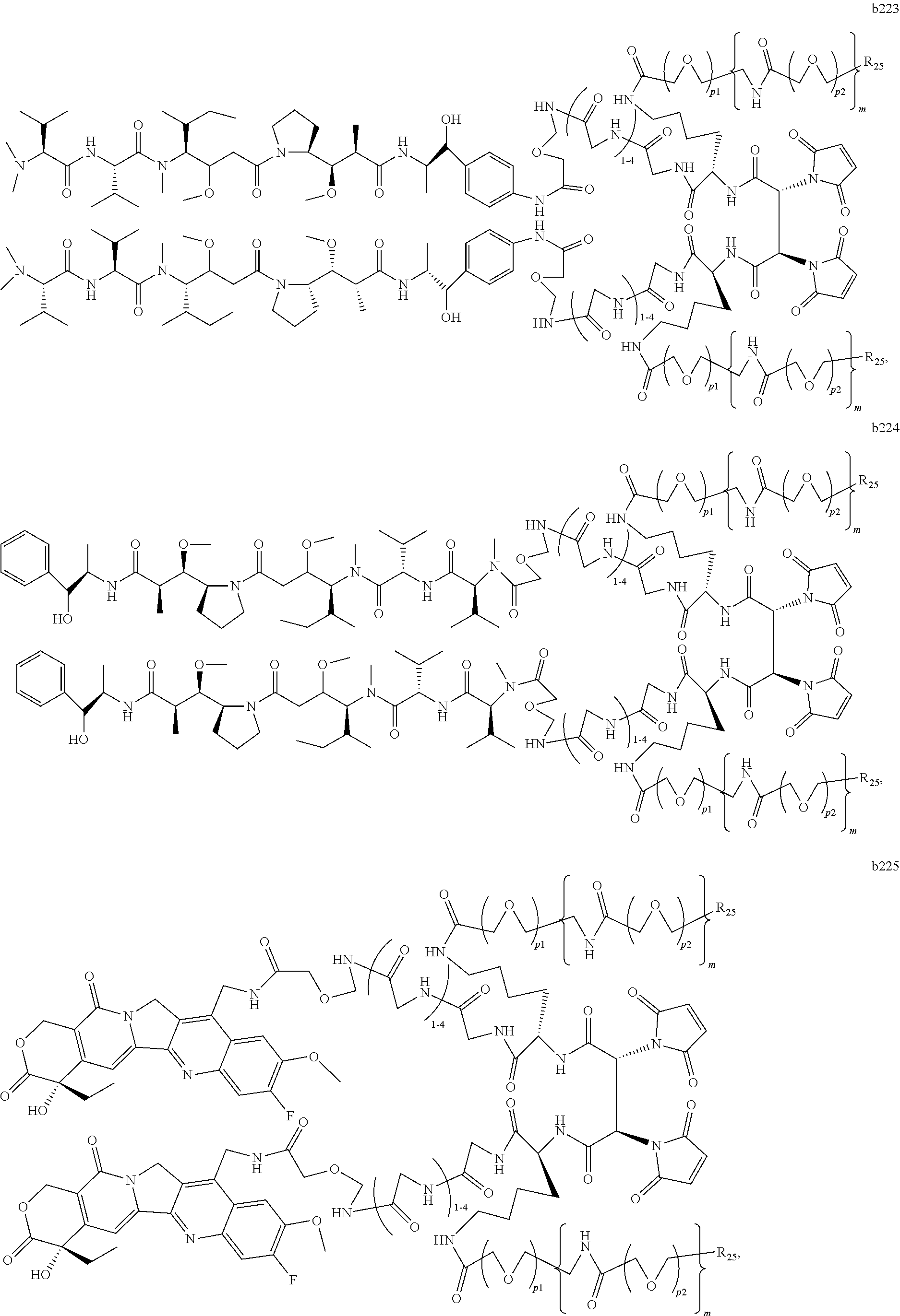

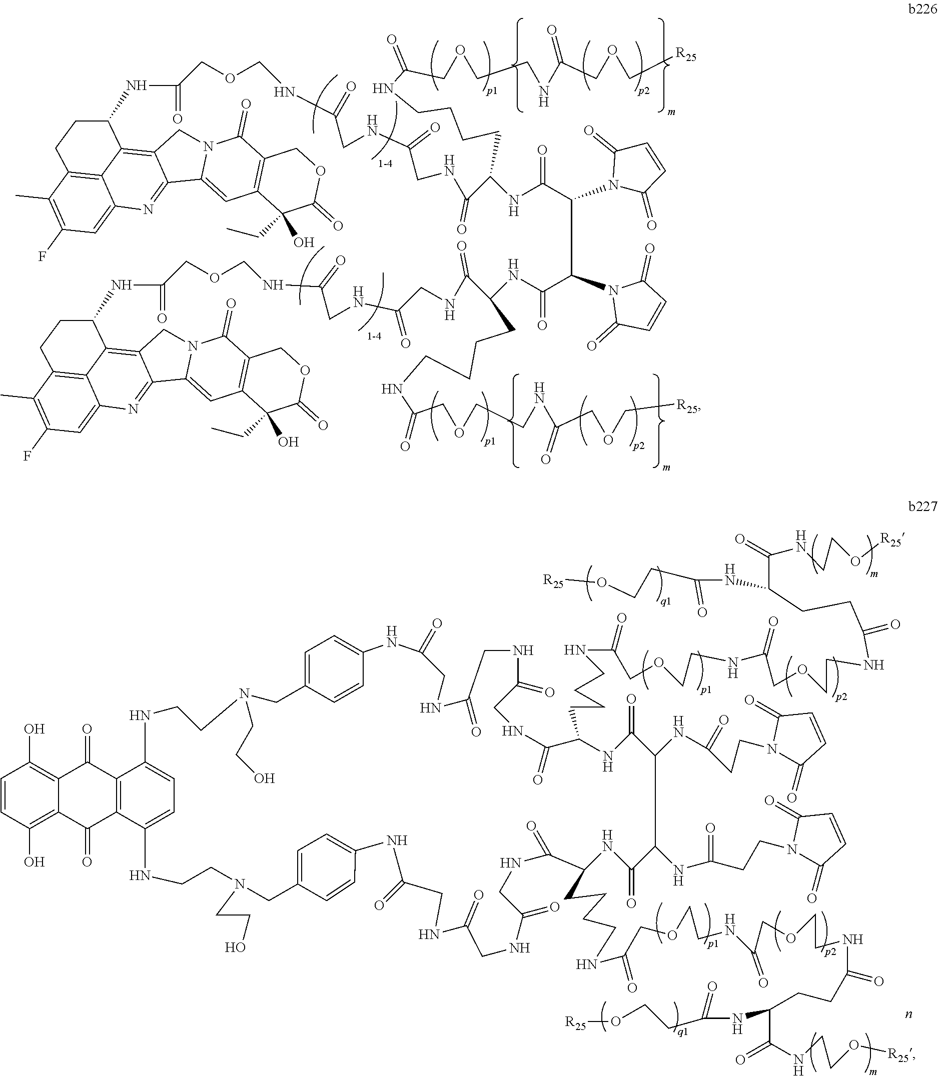

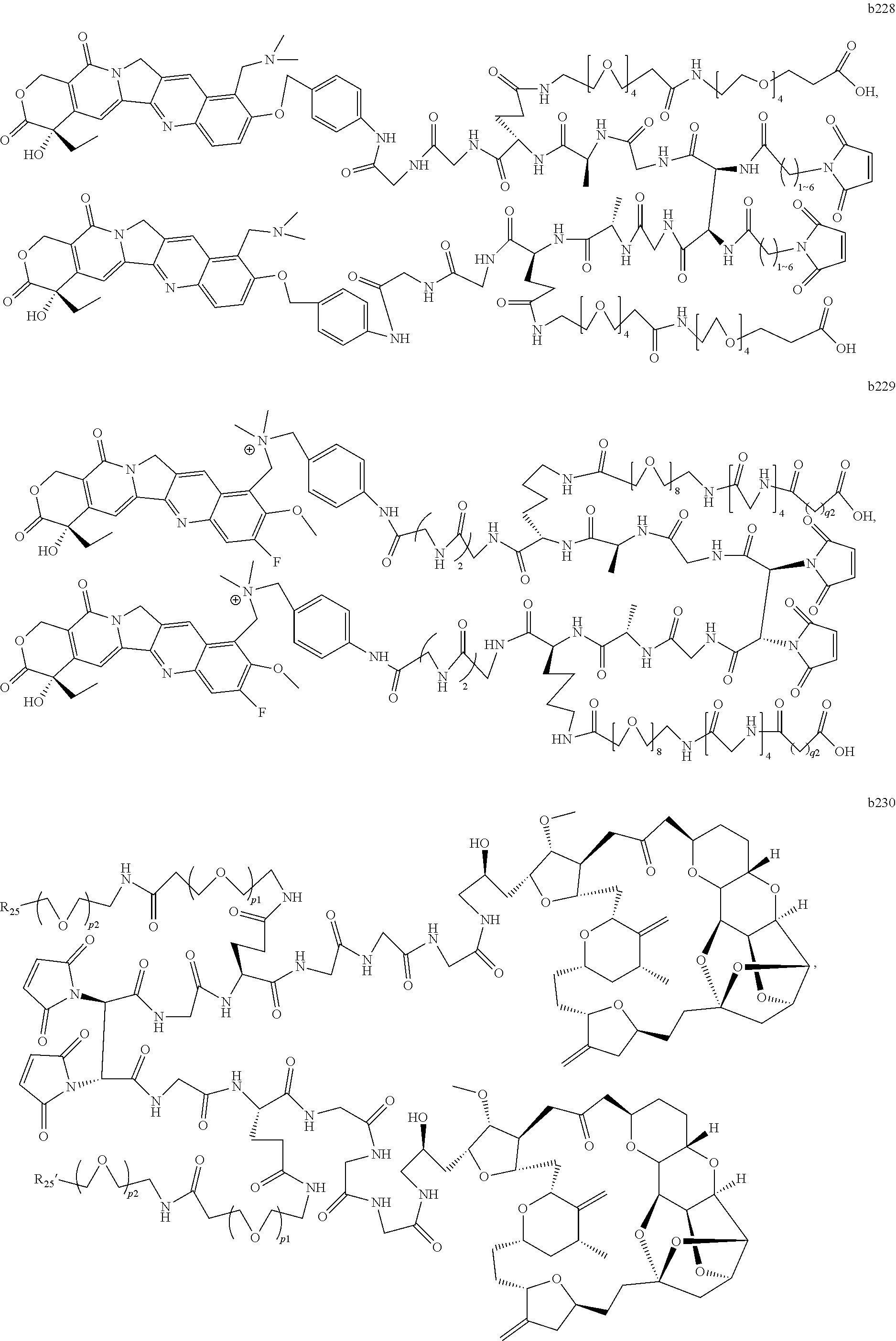

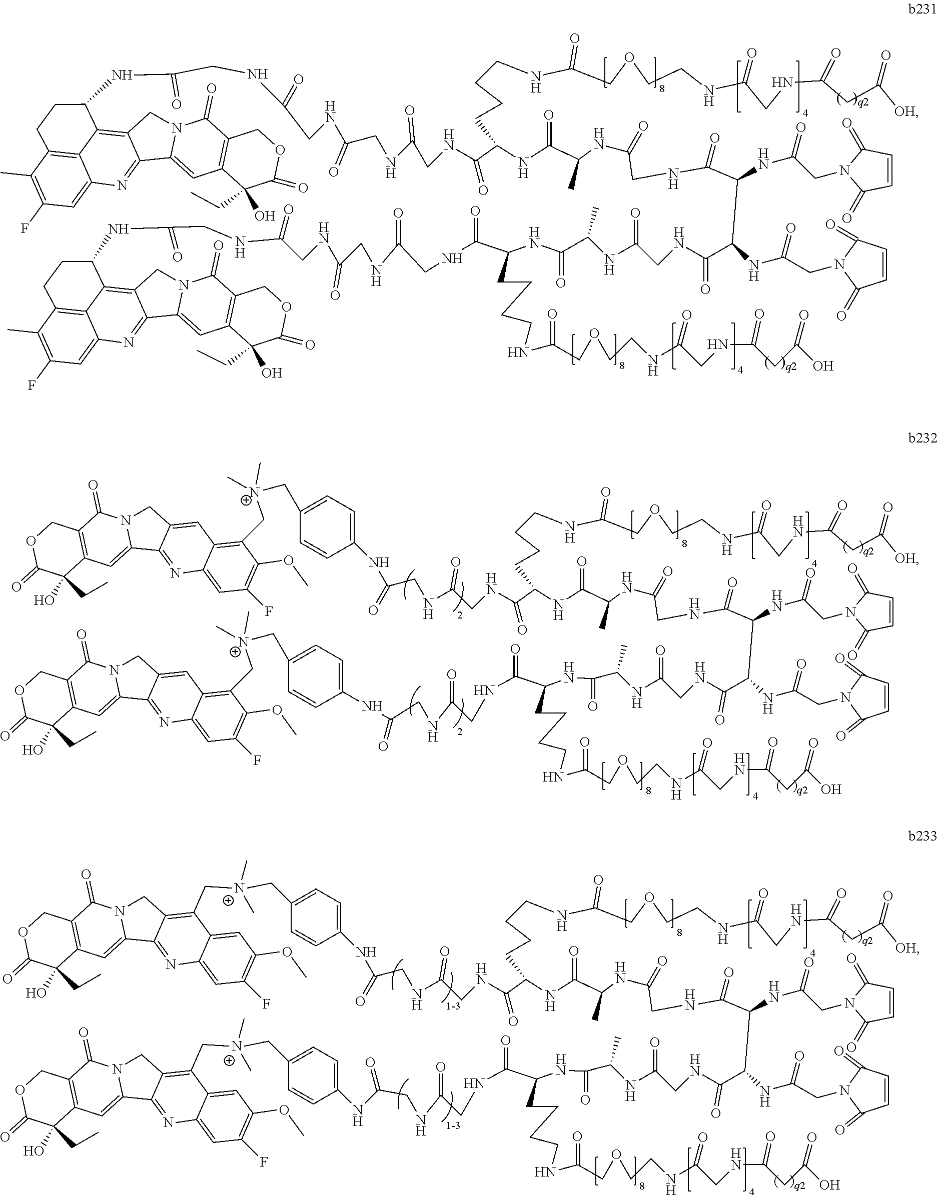

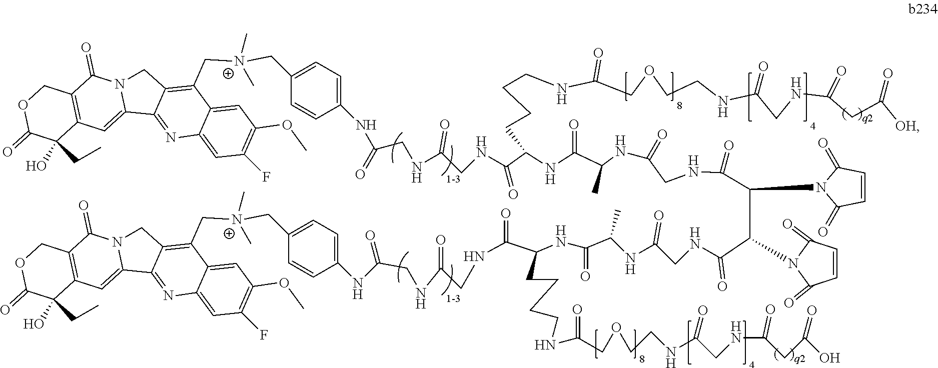

or one or more isotope of chemical elements, pharmaceutically acceptable salts, hydrates, or hydrated salts; or the polymorphic crystalline structures of these compounds; or the optical isomers, racemates, diastereomers or enantiomers; wherein m, m₁, m₂, n, P¹, R¹, R², R³, R⁴, R^{1′}, R^{2′}, R^{3′}, R₁, R₂, R₃, R₄, R₅, R₆, R₁₂, R₁₂′, R₂₅, R₂₆, R₂₅′, X₁, X₂, X₃, X₅, X₆, Y₁, Y₂, Y₆, Z₃, Z₅, p. p₁, p₂, P₃, q₁, q₂, Lv₁, Aa, (Aa)r, Ar and mAb are described the same above.

### The Antibody-Like Protein

The antibody-like protein used for the conjugation process is preferred a cell-binding antibody-like protein molecule that binds to, complexes with, or reacts with a moiety of a cell population sought to be therapeutically or otherwise biologically modified.

For convenience in this section and elsewhere, “antibody-like protein” should be understood to include “antibody-like protein and peptide” except where the context requires otherwise. Suitable antibody-like proteins which may be present in the conjugates of the invention include for example peptides, polypeptides, antibodies, antibody fragments, enzymes, cytokines, chemokines, receptors, blood factors, peptide hormones, toxin, transcription antibody-like proteins, or multimeric antibody-like proteins, wherein they have interchain disulfide bonds structurally.

Enzymes include carbohydrate-specific enzymes, proteolytic enzymes and the like, for example the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerbrosidase, and glutaminase.

Blood antibody-like proteins include albumin, transferrin, Factor VII, Factor VIII or Factor IX, von Willebrand factor, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, for example IFN-α. or IFN-β, colony stimulating factors, haemoglobin, cytokines, antibodies, antibody fragments, chorionicgonadotropin, follicle-stimulating hormone, thyroid stimulating hormone and tissue plasminogen activator.

Other antibody-like proteins of interest are allergen antibody-like proteins disclosed by Dreborg et al Crit. Rev. Therap. Drug Carrier Syst. (1990) 6 315-365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosylated interleukins, interferons and colony stimulating factors are of interest, as are immunoglobulins such as IgG, IgE, IgM, IgA, IgD and fragments thereof. Of particular interest are receptor and ligand binding antibody-like proteins and antibodies and antibody fragments which are used in clinical medicine for diagnostic and therapeutic purposes.

The antibody-like protein herein is preferred (A): the group consisting of an antibody, a antibody-like protein molecule, probody, nanobody, peptides, an antibody coating on polymeric micelle, an antibody-liposome, a lipoprotein-based drug carrier, an antibody coating on nano-particle, an antibody-dendrimer, and a particle said above coated or linked with an antibody-like protein (antibody), or a combination of said above thereof,
(B): an antibody-like protein, full-length antibodies (polyclonal antibodies, monoclonal antibodies, dimers, multimers, multispecific antibodies (e.g., bispecific antibody, trispecific antibody, or tetraspecific antibody); single chain antibodies; an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune antibody-like proteins (SIP), a lymphokine antibody-like protein, a hormone type antibody-like protein, a growth factor antibody-like protein, a colony stimulating factor antibody-like protein, a nutrient-transport antibody-like protein, large molecular weight antibody-like proteins, fusion antibody-like proteins, a kinase inhibitor antibody-like protein, gene-targeting antibody-like protein, antibody-like protein coated on nanoparticles or polymers modified with antibodies or large molecular weight antibody-like proteins;

The fragments of antibodies include Fab, Fab′, F(ab′)₂, Fᵥ, [Parham, J. Immunol. 131, 2895-902 (1983)], fragments produced by a Fab expression library, and epitope-binding fragments of any of the above which immuno-specifically bind to cancer cell antigens, viral antigens, microbial antigens or an antibody-like protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-10, GM-CSF, interferon-gamma (IFN-7); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight antibody-like proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Kohler, G.; Milstein, C. (1975). Nature 256: 495-7). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0˜20 mM glutamine, 0˜20% fetal calf serum, several ppm amount of heavy metals, such as Cu, Mn, Fe, or Zn, etc, or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82: 178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352: 264-8 (1991); Huse et al., Science 246: 1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222: 9-27 (2008); Almagro et al, Front Biosci. 13: 1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, an antibody like peptide or protein that bind/block/target or in some other way interact with the epitopes or corresponding receptors on a targeted cell can be used as a binding molecule. These antibody like peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of antibody like peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs via the linkers of this prevention for treating cancer, autoimmune disease, and/or infectious disease include, but are not limited to, 3F₈ (anti-GD2), Abagovomab (anti CA-125), Abciximab (anti CD41 (integrin alpha-JIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (a chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (α chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R₂), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin a4), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R a), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C11 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 or DLL4 (delta-like-3 or delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF1OB (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The antibody-like protein, more preferred an IgG antibody that is able to against tumor cells, virus infected cells, microorganism infected cells, parasite infected cells, autoimmune disease cells, activated tumor cells, myeloid cells, activated T-cells, an affecting B cells, or melanocytes. More specifically the antibody is able to against abnormal cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158bl, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213al, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11(C—C motif chemokine 11), CCR4 (C—C chemokine receptor type 4), CCR5, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor a-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α6β4, α7β7, αllβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUCl(Mucin 1, cell surface associated (MUC1) or polymorphic epithelial mucin (PEM)), MUC1-KLH, MUC16 (CA125), MCP1(monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY—BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1(glycoprotein 75), TRP-1 (Trop1), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

In another specific embodiment, the antibody-drug conjugates of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the the antibody-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjogren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome In another specific embodiment, the antibody-drug conjugates of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial(M2) antibody; Rheumatoid factor antibody; Anti-MCV antibody; Anti-topoisomerase antibody; Anti-neutrophil cytoplasmic(cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF—R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimi-dase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The antibody-drug conjugates of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The cell binding molecule, which is more preferred to be an antibody described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)-Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157:H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as cell binding ligands used in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

### Formulation and Application

The conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L ˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammounium, or trihydroxyethylamino salts of citric acid, ascorbic acid, gluconic acid, carbonic acid, tartaric acid, succinic acid, acetic acid or phtalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0%-20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc); etc. Preferred surfactants are polyoxyethylene sorbitan fatty acid esters e.g. polysorbate 20, 40, 60 or 80 (Tween 20, 40, 60 or 80). The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenoxyl, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0%-5.0% by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0%-30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄₀H, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-p-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenoxyl, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 6 months at 25° C. More preferred a conjugate in either liquid or loyphilizate formulation should exhibit a shelf life of more than 12 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody(protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 24 month, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8-54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 25 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/triweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferrably the unit dose range is from 1 to 500 mg administered one to four times a month and even more preferably from 1 mg to 100 mg, once a week, or once a biweek, or once a triweek. Conjugatess provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21^{st} ed.; Lippincott Williams & Wilkins: Philadelphia, PA, 2005.

The formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination.

Liquid preparations for parenteral administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In yet another embodiment, a pharmaceutical composition comprising a therapeutically effective amount of the conjugate of Formula (V), (VI), (VII), or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic drugs or radiation therapy can be administered prior or subsequent to administration of a conjugate, in one aspect at least an hour, 12 hours, a day, a week, biweeks, triweeks, a month, in further aspects several months, prior or subsequent to administration of a conjugate of the invention.

The synergistic agents are preferably selected from one or several of the following drugs: The synergistic agents according to Claim 20 are selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Poziotinib Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.

The drugs/cytotoxic agents used for conjugation of the present patent can be any analogues and/or derivatives of drugs/molecules described in the present patent. One skilled in the art of drugs/cytotoxic agents will readily understand that each of the drugs/cytotoxic agents described herein can be modified in such a manner that the resulting compound still retains the specificity and/or activity of the starting compound. The skilled artisan will also understand that many of these compounds can be used in place of the drugs/cytotoxic agents described herein. Thus, the drugs/cytotoxic agents of the present invention include analogues and derivatives of the compounds described herein.

According to a still further object, the conjugate and process of the present invention may be prepared in a number of ways well known to those skilled in the art. The Camptothecin analogs used in the conjugate can be synthesized, for example, by application or adaptation of the methods described below, or variations thereon as appreciated by the skilled artisan. The appropriate modifications and substitutions will be readily apparent and well known or readily obtainable from the scientific literature to those skilled in the art. In particular, such methods can be found in R. C. Larock, Comprehensive Organic Transformations, 2^{nd} Edition, Wiley-VCH Publishers, 1999.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see P. G. Wuts and T.W. Greene, Greene's Protective Groups in Organic Synthesis, Wiley-Interscience; 4th edition (2006). Some reactions may be carried out in the presence of a base, or an acid or in a suitable solvent. There is no particular restriction on the nature of the base, acid and solvent to be used in this reaction, and any base, acid or solvent conventionally used in reactions of this type may equally be used here, provided that it has no adverse effect on other parts of the molecule. The reactions can take place over a wide range of temperatures. In general, we find it convenient to carry out the reaction at a temperature of from −80° C. to 150° C. (more preferably from about room temperature to 100° C.). The time required for the reaction may also vary widely, depending on many factors, notably the reaction temperature and the nature of the reagents. However, provided that the reaction is effected under the preferred conditions outlined above, a period of from 3 hours to 20 hours will usually suffice.

The work-up of the reaction can be carried out by conventional means. For example, the reaction products may be recovered by distilling off the solvent from the reaction mixture or, if necessary after distilling off the solvent from the reaction mixture, pouring the residue into water followed by extraction with a water-immiscible organic solvent and distilling off the solvent from the extract. Additionally, the product can, if desired, be further purified by various well known techniques, such as recrystallization, reprecipitation or the various chromatography techniques, notably column chromatography or preparative thin layer chromatography.

The process of the invention is further illustrated but not restricted by the description in the following examples. All references cited herein and in the examples that follow are expressly incorporated by reference in their entireties.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Acadmy of Science, unless otherwise specified. Cell culture reagents were obtained from Invitrogen Corp., unless otherwise specified. All anhydrous solvents were commercially obtained and stored in Sure-seal bottles under nitrogen. PEG compounds were purchased from Biomatrik Inc, Jiaxing, China. Some chemical compounds, when were not referred synthesis from, were provided by CROs (e. g. Wuxi Apptec, Haoyuan Chemexpress, Raybow Pharma) in China. Experimental animals were purchased from National Resource Center of Model Mice via GemPharmatech. Co., Ltd, Najing, China and Shanghai SLAC Laboratory Animal Co., Ltd., Shanghai, China; T-DM1 was purchased from Roche via a pharmacy in Hong Kong, China. All other reagents and solvents were purchased as the highest grade available and used without further purification. The preparative HPLC separations were performed with Varain PreStar HPLC. HPLC analysis was conducted on Agilent 1260. The mass spectral data were acquired on a Waters Xevo QTOF mass spectrum equipped with Waters Acquity UPLC separations module and Acquity TUV detector. NMR spectra were recorded on Zhongke-niujin WNMR-I 400 MHz instrument at the Department of Chemistry of Zhejiang Sci-Tech University. Chemical shifts (6) are reported in parts per million (ppm) referenced to tetramethylsilane at 0.00 and coupling constants (J) are reported in Hz. The elemental analysis of C, H, and/or N was provided by the Department of Chemistry of Zhejiang Sci-Tech University and conducted on Elementar UNICUBE. Quantitative analysis of metal atoms was performed on Agilent ICPOES 730 ICP-MS.

### Example 1. Synthesis of zinc propan-2-amine chloride complex (Z-01)

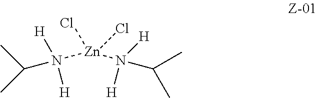

zinc chloride (6.0 g, 44.03 mmol) was dissolved into 50 mL methanol and cooled to about 5° C. in an ice-water bath. Propan-2-amine (10.4 g, 176.11 mmol) dissolved in 60 mL methanol was added dropwise to the methanol solution of zinc chloride, while the solution temperature was maintained below 5° C. In the process of dripping of the amine solution, there was white solid precipitation slowly. After dripping, the solution was warmed to room temperature and stirred slowly overnight to have much more precipitation of white solids. Then 200 mL of ethyl acetate was added to the mixture and the mixture was kept to stir for 10 more minutes. The resulting white solid was filtered, washed with methanol and then dried over vacuum pump to give 8.9 g of zinc propan-2-amine chloride complex as an off-white solid, 79.4% yield. ¹HNMR (400 MHz, DMSO-d6): δ=3.61 (s, 4H), 3.12-3.06 (m, 2H), 1.12 (d, J=6.3 Hz, 12H). Elemental analysis, calcd.: C, 28.32; H, 7.13; N, 11.01; found: C, 28.08; H, 7.10; N, 11.20;

### Example 2. Synthesis of Zinc Ethanolamine Chloride Complex (Z-02)

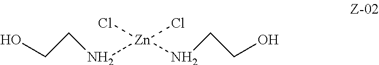

The procedure is the same as that of Example 1, starting from zinc chloride (6.0 g, 44.03 mmol) and ethanolamine (10.8 g, 176.11 mmol) to provide 8.2 g of zinc ethanolamine chloride complex as an off-white solid, 72.6% yield. ¹H NMR (400 MHz, DMSO) δ 3.77 (s, 2H), 3.50 (t, J=5.6 Hz, 2H), 2.69 (t, J=5.7 Hz, 2H). Elemental analysis, calcd.: C, 18.59; H, 5.46; N, 10.84; found: C, 18.25; H, 5.02; N, 10.30.

### Example 3. Synthesis of Zinc Diethanolamine Chloride Complex (Z-03)

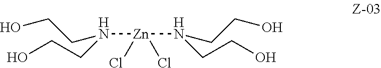

The procedure is the same as that of Example 1, starting from zinc chloride (6.0 g, 44.03 mmol) and diethanolamine (18.5 g, 176.11 mmol) to provide 12.2 g of zinc diethanolamine chloride complex as an off-white solid, 80.4% yield. ¹H NMR (400 MHz, DMSO) δ 4.60 (s, 2H), 3.59 (m, 8H), 3.35 (m, 4H), 2.72 (s, 8H). Elemental analysis, calcd.: C, 27.73; H, 6.40; N, 8.08; found: C, 27.25; H, 6.05; N, 7.80;

### Example 4. Synthesis of Zinc Homopiperazine Chloride Complex (Z-04)

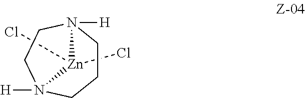

The procedure is the same as that of Example 1, starting from zinc chloride (6.0 g, 44.03 mmol) and Piperazine (15.2 g, 176.11 mmol) to provide 9.2 g of zinc homopiperazine chloride complex as an off-white solid, 73.6% yield. ¹H NMR (400 MHz, DMSO) δ 4.34 (s, 2H), 3.01-2.85 (m, 8H), 1.87-1.75 (m, 2H). Elemental analysis, calcd.: N, 11.85; found: N, 11.50;

### Example 5. Synthesis of zinc piperazine chloride complex (Z-05)

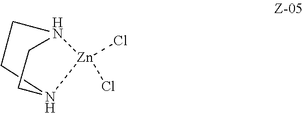

The procedure is the same as that of Example 1, starting from zinc chloride (6.0 g, 44.03 mmol) and piperazine (15.2 g, 176.11 mmol) to provide 7.6 g of zinc piperazine chloride complex as an off-white solid, 94.0% yield. ¹FH NMR (400 MHz, DMSO) δ 2.73 (s, 8H). Elemental analysis, calcd.: C, 21.60; H, 4.53; N, 12.59; found: C, 21.10; H, 4.69; N, 12.30;

### Example 6. Synthesis of zinc o-phenylenediamine chloride complex (Z-06)

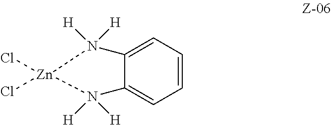

The procedure is the same as that of Example 1, starting from zinc chloride (6.0 g, 44.03 mmol) and O-phenylenediamine (19.1 g, 176.11 mmol) to provide 9.5 g of zinc o-phenylenediamine chloride complex as an off-white solid, 88.3% yield. ¹HNMR (400 MHz, DMSO-d6): δ=6.55-6.51 (m, 2H), 6.43-6.39 (m, 2H), 4.43 (s, 4H). Elemental analysis, calcd.: C, 29.48; H, 3.30; N, 11.46; found: C, 29.70; H, 3.63; N, 11.30.

### Example 7. Synthesis of zinc propylenediamine chloride complex (Z-07)

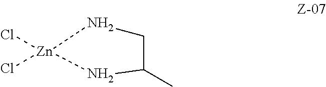

The procedure is the same as that of Example 1, starting from zinc chloride (6.0 g, 44.03 mmol) and propylenediamine (13.0 g, 176.11 mmol) to provide 8.1 g of zinc propylenediamine chloride complex as an off-white solid, 87.4% yield. ¹H NMR (400 MHz, DMSO) δ 3.93 (s, 2H), 3.82 (s, 2H), 2.86-2.66 (m, 2H), 2.24 (s, 1H), 1.09 (d, J=6.5 Hz, 3H). Elemental analysis, calcd. for C₃HiOCl₂N₂Zn (207.95): C, 17.12; H, 4.79; Cl, 33.70; N, 13.31; Zn, 31.07

### Example 8. Synthesis of zinc 1, 2-cyclohexanediamine chloride complex (Z-08)

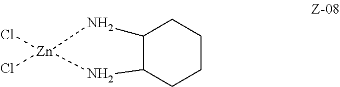

The procedure is the same as that of Example 1, starting from zinc chloride (6.0 g, 44.03 mmol) and 1, 2-cyclohexanediamine (20.1 g, 176.11 mmol) to provide 8.5 g of zinc 1, 2-cyclohexanediamine chloride complex as an off-white solid, 77.7% yield. ¹H NMR (400 MHz, DMSO) δ 3.89 (s, 4H), 2.27-2.11 (m, 2H), 1.65 (d, J=9.9 Hz, 2H), 1.59-1.45 (m, 2H), 1.32-1.26 (m, 2H), 1.19-1.14 (m, 2H). Elemental analysis, calcd. for C₆H₁₄Cl₂N₂Zn (247.98): N, 11.18; Zn, 26.10, found N, 10.88; Zn, 25.95.

### Example 9. Synthesis of zinc methylamine chloride complex (Z-09)

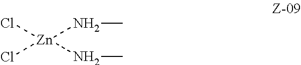

The procedure is the same as that of Example 1, starting from zinc chloride (6.0 g, 44.03 mmol) and methylamine hydrochloride (20.1 g, 176.11 mmol) to provide 9.5 g of zinc methylamine chloride complex as an off-white solid, 94.0% yield. ¹FH NMR (400 MHz, DMSO) δ 7.71 (s, 6H), 2.37 (d, J=3.6 Hz, 6H). Elemental analysis, calcd. for C₂HiOCl₂N₂Zn (195.95): N, 14.12; Zn, 32.95, found N, 13.96; Zn, 32.82.

### Example 10. Synthesis of zinc ethylamine chloride complex (Z-10)

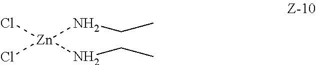

The procedure is the same as that of Example 1, starting from zinc chloride (6.0 g, 44.03 mmol) and ethylamine solution (19.9 g, 176.11 mmol, 40% w.t) to provide 8.3 g of zinc ethylamine chloride complex as an off-white solid, 84.0% yield. ¹H NMR (400 MHz, DMSO) δ 3.76-3.50 (m, 4H), 2.66 (q, J=7.2 Hz, 4H), 1.10 (t, J=7.2 Hz, 6H). Elemental analysis, calcd. for C₄H₁₄Cl₂N₂Zn (223.98): N, 12.37; Zn, 28.87, found N, 12.08; Zn, 28.72.

### Example 11. Synthesis of zinc 2-methylpropane-1, 2-diamine chloride complex (Z-11)

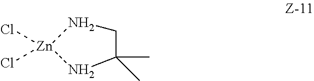

The procedure is the same as that of Example 1, starting from zinc chloride (6.0 g, 44.03 mmol) and 2-methylpropane-1, 2-diamine (15.5 g, 176.11 mmol) to provide 7.8 g of zinc 2-methylpropane-1, 2-diamine chloride complex as an off-white solid, 79.6% yield. ¹FH NMR (400 MHz, DMSO) δ 4.02 (s, 2H), 3.78 (s, 2H), 2.43 (d, J=5.6 Hz, 2H), 1.12 (s, 6H). Elemental analysis, calcd. for C₄H₁₂Cl₂N₂Zn (221.97): N, 12.48; Zn, 28.80; Found: N, 12.35; Zn, 28.58.

### Example 12. Synthesis of zinc (3R, 4S)-tetrahydrofuran-3, 4-diamine chloride complex (Z-12)

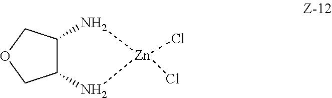

The procedure is the same as that of Example 1, starting from zinc chloride (2.0 g, 14.68 mmol) and (3R, 4S)-tetrahydrofuran-3, 4-diamine (5.1 g, 29.35 mmol) to provide 3.8 g of zinc (3R, 4S)-tetrahydrofuran-3, 4-diamine chloride complex as an off-white solid, 83.7% yield. ¹H NMR (400 MHz, DMSO) δ 8.82 (s, 6H), 4.07-3.83 (m, 6H). Elemental analysis, calcd. for C₄HiOCl₂N₂OZn (235.95): Zn, 27.42; N, 12.48; Found: N, 12.30; Zn, 27.26.

### Example 13. Synthesis of zinc pyrrolidine chloride complex (Z-13)

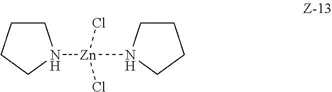

The procedure is the same as that of Example 1, starting from zinc chloride (6.0 g, 44.03 mmol) and pyrrolidine (15.5 g, 176.11 mmol) to provide 11.0 g of zinc pyrrolidine chloride complex as an off-white solid, 90.4% yield. ¹FH NMR (400 MHz, DMSO) δ 4.46 (s, 2H), 2.85 (t, J=6.2 Hz, 8H), 1.81-1.65 (m, 8H). Elemental analysis, calcd. for C₈H₁₈Cl₂N₂Zn (276.01): N, 10.06; Zn, 23.47. Found: N, 9.88; Zn, 23.59.

### Example 14. Synthesis of zinc N-methylimidazole chloride complex (Z-14)

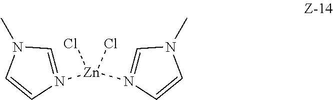

The procedure is the same as that of Example 1, starting from zinc chloride (6.0 g, 44.03 mmol) and N-methylimidazole (14.5 g, 176.11 mmol) to provide 10.0 g of zinc homopiperazine chloride complex as an off-white solid, 90.4% yield. ¹FH NMR (400 MHz, DMSO) δ 8.09 (s, 2H), 7.39 (d, J=1.4 Hz, 2H), 7.06 (t, J=1.3 Hz, 2H), 3.77 (s, 6H). Elemental analysis, calcd. for C₈H₁₂Cl₂N₄Zn (297.97): N, 18.64; Zn, 21.76; Found: N, 18.40; Zn, 21.58.

### Example 15. Synthesis of zinc piperidine chloride complex (Z-15)

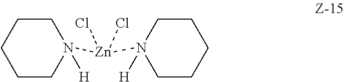

The procedure is the same as that of Example 1, starting from zinc chloride (6.0 g, 44.03 mmol) and piperidine (15.0 g, 176.11 mmol) to provide 10.5 g of zinc piperidine chloride complex as an off-white solid, 77.8% yield. ¹H NMR (400 MHz, DMSO) δ 4.00 (s, 2H), 2.80 (t, J=4.6 Hz, 8H), 1.61-1.45 (m, 12H). Elemental analysis, calcd. for C₁₀H₂₂Cl₂N₂Zn (304.04): N, 9.14; Zn, 21.33; Found: N, 8.83; Zn, 21.50.

### Example 16. Synthesis of zinc pyridine chloride complex (Z-16)

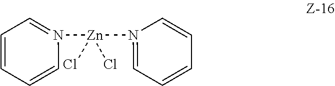

The procedure is the same as that of Example 1, starting from zinc chloride (6.0 g, 44.03 mmol) and pyridine (13.9 g, 176.11 mmol) to provide 9.5 g of zinc pyridine chloride complex as an off-white solid, 73.2% yield. ¹H NMR (400 MHz, DMSO) δ 8.61 (dt, J=4.4, 1.7 Hz, 4H), 7.94-7.86 (m, 2H), 7.49 (ddd, J=7.6, 4.4, 1.5 Hz, 4H). Elemental analysis: calcd. for C₁₀H₁₀C₁₂N₂Zn (291.95): N, 9.51; Zn, 22.20. Found: N, 9.35, Zn, 21.90.

### Example 17. Synthesis of zinc 2-methylpyridine chloride complex (Z-17)

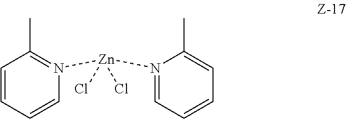

The procedure is the same as that of Example 1, starting from zinc chloride (6.0 g, 44.03 mmol) and 2-methylpyridine (16.1 g, 176.11 mmol) to provide 11.3 g of zinc 2-methylpyridine chloride complex as an off-white solid, 79.6% yield. ¹H NMR (400 MHz, DMSO) δ 8.48 (ddd, J=5.0, 1.9, 0.9 Hz, 2H), 7.72 (td, J=7.7, 1.9 Hz, 2H), 7.29 (d, J=7.8 Hz, 2H), 7.22 (ddd, J=7.7, 5.4, 1.2 Hz, 2H), 2.50 (s, 6H). Elemental analysis: calcd. for C₁₂H₁₄Cl₂N₂Zn (319.98): N, 8.69; Zn, 20.27; Found: N, 8.35, Zn, 20.12.

### Example 18. Synthesis of zinc 6-methylpyridin-2-amine chloride complex (Z-18)

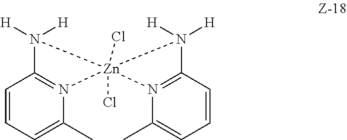

The procedure is the same as that of Example 1, starting from zinc chloride (4.0 g, 29.35 mmol) and 6-methylpyridin-2-amine (12.7 g, 117.41 mmol) to provide 2.3 g of zinc 6-methylpyridin-2-amine chloride complex as an off-white solid, 32.0% yield. ¹FH NMR (400 MHz, DMSO) δ 7.25 (dd, J=8.2, 7.2 Hz, 1H), 6.34 (d, J=7.2 Hz, 1H), 6.23 (d, J=8.2 Hz, 1H), 5.75 (s, 2H), 2.23 (s, 3H). Elemental analysis: calcd. for C₆H₈Cl₂N₂Zn (241.94): N, 11.46; Zn, 26.75; Found: N, 11.25, Zn, 26.48.

### Example 19. Synthesis of zinc morpholine chloride complex (Z-19)

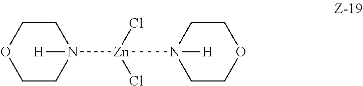

The procedure is the same as that of Example 1, starting from zinc chloride (6.0 g, 44.03 mmol) and morpholine (15.2 g, 176.11 mmol) to provide 8.1 g of zinc morpholine chloride complex as an off-white solid, 89.5% yield. ¹H NMR (400 MHz, DMSO) δ 3.64-3.59 (m, 8H), 2.79 (dd, J=5.6, 4.0 Hz, 8H). Chemical formula: C₈H₁₈Cl₂N₂O₂Zn; exact mass: 308.0037; elemental analysis: N, 9.02; Zn, 21.05, found: N, 8.81; Zn, 20.75.

### Example 20. Synthesis of zinc methylpiperazine chloride complex (Z-20)

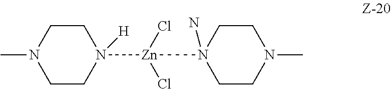

The procedure is the same as that of Example 1, starting from zinc chloride (4.0 g, 29.35 mmol) and methylpiperazine (11.8 g, 117.41 mmol) to provide 8.1 g of zinc methylpiperazine chloride complex as an off-white solid, 82.5% yield. ¹H NMR (400 MHz, DMSO) δ 2.81 (t, J=5.0 Hz, 8H), 2.37 (s, 8H), 2.15 (s, 6H). Chemical formula: C₁₀H₂₄Cl₂N₄Zn; exact mass: 334.0669; elemental analysis: N, 16.64; Zn, 19.42, found: N, 16.80; Zn, 18.98.

### Example 21. Synthesis of zinc pyridin-2-ylmethanamine chloride complex (Z-21)

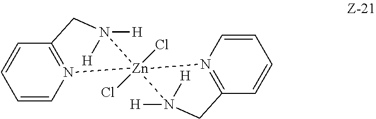

The procedure is the same as that of Example 1, starting from zinc chloride (4.0 g, 29.35 mmol) and pyridin-2-ylmethanamine (12.7 g, 117.41 mmol) to provide 6.2 g of zinc pyridin-2-ylmethanamine chloride complex as an off-white solid, 86.8% yield. ¹H NMR (400 MHz, DMSO) δ 8.55 (dt, J=5.2, 1.4 Hz, 1H), 7.99 (td, J=7.7, 1.7 Hz, 1H), 7.58-7.53 (m, 1H), 7.51 (ddd, J=7.5, 5.2, 1.2 Hz, 1H), 4.09 (s, 2H), 4.06 (s, 2H). Chemical formula: C₁₂H₁₆Cl₂N₄Zn; exact mass: 350.0043; elemental analysis: N, 15.89; Zn, 18.54, found: N, 16.21; Zn, 18.72.

### Example 22. Synthesis of zinc 4-methylthiazole chloride complex (Z-22)

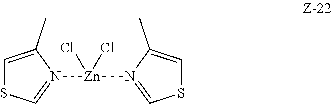

The procedure is the same as that of Example 1, starting from zinc chloride (4.0 g, 29.35 mmol) and 4-methylthiazole (11.6 g, 117.41 mmol) to provide 6.5 g of zinc 4-methylthiazole chloride complex as an off-white solid, 66.2% yield. ¹FH NMR (400 MHz, DMSO) δ 9.02 (d, J=2.0 Hz, 2H), 7.33 (dt, J=2.0, 1.0 Hz, 2H), 2.43 (d, J=1.0 Hz, 6H).

### Example 23. Synthesis of zinc 4-methylbenzene-1, 2-diamine chloride complex (Z-23)

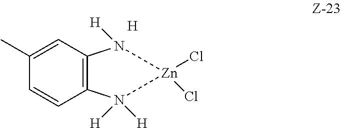

The procedure is the same as that of Example 1, starting from zinc chloride (4.0 g, 29.35 mmol) and 4-methylbenzene-1, 2-diamine (14.3 g, 117.41 mmol) to provide 5.8 g of zinc 4-methylbenzene-1, 2-diamine chloride complex as an off-white solid, 77.0% yield. ¹H NMR (400 MHz, DMSO) δ 6.42 (d, J=7.7 Hz, 1H), 6.36 (d, J=2.0 Hz, 1H), 6.21 (dd, J=7.7, 2.0 Hz, 1H), 4.30 (s, 4H), 2.08 (s, 3H). Chemical formula: C₇H₁₀Cl₂N₂Zn; exact mass: 255.9513; elemental analysis: N, 10.84; Zn, 25.30; found: N, 10.42; Zn, 25.73.

### Example 24. Synthesis of zinc butane-2, 3-diamine chloride complex (Z-24)

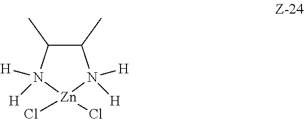

The procedure is the same as that of Example 1, starting from zinc chloride (800 mg, 5.87 mmol) and butane-2, 3-diamine (939.2 mg, 5.87 mmol) to provide 1.90 g of zinc butane-2, 3-diamine chloride complex as an off-white solid, 87.1% yield. ¹H NMR (400 MHz, DMSO) δ 8.61 (s, 5H), 3.54-3.47 (m, 2H), 1.29 (d, J=6.5 Hz, 6H).

### Example 25. Synthesis of zinc oxazole chloride complex (Z-25)

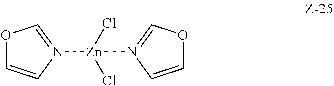

The procedure is the same as that of Example 1, starting from zinc chloride (4.0 g, 29.35 mmol) and oxazole (8.1 g, 117.41 mmol) to provide 7.8 g of zinc oxazole chloride complex as an off-white solid, 96.8% yield. ¹H NMR (400 MHz, DMSO) δ 8.41 (d, J=0.9 Hz, 2H), 8.16 (t, J=0.9 Hz, 2H), 7.27 (d, J=0.9 Hz, 2H). Chemical formula: C₆H₆Cl₂N₂O₂Zn; exact mass: 271.9098; elemental analysis: N, 10.21; Zn, 23.83; found: N, 9.91; Zn, 23.70.

### Example 26. Synthesis of zinc thiazole chloride complex (Z-26)

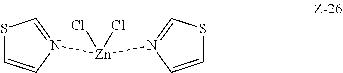

The procedure is the same as that of Example 1, starting from zinc chloride (4.0 g, 29.35 mmol) and thiazole (10.1 g, 117.41 mmol) to provide 6.5 g of zinc thiazole chloride complex as an off-white solid, 72.3% yield. ¹H NMR (400 MHz, DMSO) δ 9.17 (d, J=1.9 Hz, 2H), 7.99 (d, J=3.2 Hz, 2H), 7.82 (dd, J=3.2, 1.9 Hz, 2H). Chemical formula: C₆H₆Cl₂N₂S₂Zn; exact mass: 303.8641; elemental analysis: N, 9.14; Zn, 21.33; found: N, 8.96; Zn, 20.94.

### Example 27. Synthesis of zinc 2-chlorothiazole chloride complex (Z-27)

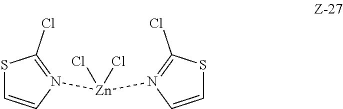

The procedure is the same as that of Example 1, starting from zinc chloride (4.0 g, 29.35 mmol) and 2-chlorothiazole (14.0 g, 117.41 mmol) to provide 7.0 g of zinc 2-chlorothiazole chloride complex as an off-white solid, 63.5% yield. ¹H NMR (400 MHz, DMSO) δ 7.78 (d, J=3.6 Hz, 2H), 7.72 (d, J=3.6 Hz, 2H). Chemical formula: C₆H₄Cl₄N₂S₂Zn; exact mass: 371.7861; elemental analysis: N, 7.46; Zn, 17.41; found: N, 7.11; Zn, 17.72.

### Example 28. Synthesis of zinc 4-methyloxazole chloride complex (Z-28)

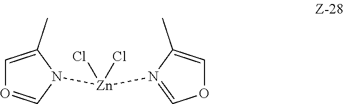

The e same as that of Example 1, starting from zinc chloride (4.0 g, 29.35 mmol) and 4-methyloxazole (9.8 g, 117.41 mmol) to provide 6.0 g of zinc homopiperazine chloride complex as an off-white solid, 67.5% yield. ¹H NMR (400 MHz, DMSO) δ 8.26 (s, 2H), 7.82 (p, J=1.2 Hz, 2H), 2.11 (d, J=1.3 Hz, 6H). Elemental anal.: calcd. for C₈H₁₀Cl₂N₂O₂Zn (299.94): N, 9.34. found: N, 8.95.

### Example 29. Synthesis of zinc 2-acetylpyridine chloride complex (Z-29)

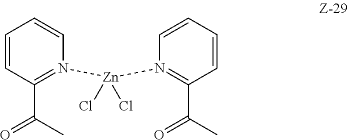

The procedure is the same as that of Example 1, starting from zinc chloride (4.0 g, 29.35 mmol) and 2-acetylpyridine (14.2 g, 117.41 mmol) to provide 9.0 g of zinc 2-acetylpyridine chloride complex as an off-white solid, 81.0% yield. ¹H NMR (400 MHz, DMSO) δ 8.75 (ddd, J=4.7, 1.7, 1.0 Hz, 1H), 8.05-7.95 (m, 2H), 7.68 (ddd, J=7.3, 4.7, 1.5 Hz, 1H), 2.65 (s, 3H).

### Example 30. Synthesis of zinc N, N-dimethylpyridin-4-amine chloride complex (Z-30)

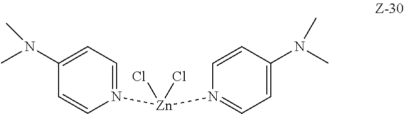

The procedure is the same as that of Example 1, starting from zinc chloride (4.0 g, 29.35 mmol) and 4-dimethylaminopyridine (7.2 g, 58.70 mmol) to provide 9.1 g of zinc 4-dimethylaminopyridine chloride complex as an off-white solid, 81.5% yield. ¹H NMR (400 MHz, DMSO-d6) δ 8.08-8.02 (m, 4H), 6.80-6.76 (m, 4H), 3.04 (s, 12H). Elemental anal. calcd. for C₁₄H₂₀Cl₂N₄Zn: Zn, 16.80; N, 14.71. Found: Zn, 17.23; N, 14.80.

### Example 31. Synthesis of zinc 4-(pyrrolidin-1-yl)pyridine chloride complex (Zn-31)

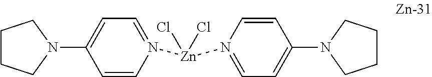

The procedure is the same as that of Example 1, starting from zinc chloride (4.0 g, 29.35 mmol) and 4-(pyrrolidin-1-yl)pyridine (7.2 g, 58.70 mmol) to provide 8.5 g of zinc 4-dimethylaminopyridine chloride complex as an off-white solid, 66.8% yield. ¹H NMR (400 MHz, DMSO-d6) δ 8.07-8.01 (m, 4H), 6.66-6.59 (m, 4H), 2.02-1.93 (m, 8H).

### Example 32. Synthesis of zinc 1-(pyridin-2-yl)ethan-1-amine chloride complex (Zn-32)

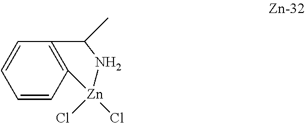

The procedure is the same as that of Example 1, starting from zinc chloride (4.0 g, 29.35 mmol) and 1-(pyridin-2-yl)ethan-1-amine (3.6 g, 29.35 mmol) to provide 6.6 g of zinc 1-(pyridin-2-yl)ethan-1-amine chloride complex as an off-white solid, 87.1% yield. 1H NMR (400 MHz, DMSO-d6) δ 8.61 (dt, J=5.0, 1.4 Hz, 1H), 8.10 (td, J=7.7, 1.7 Hz, 1H), 7.67 (dt, J=8.1, 1.1 Hz, 1H), 7.62 (ddd, J=7.5, 5.2, 1.2 Hz, 1H), 4.51 (s, 2H), 4.43 (q, J=6.8 Hz, 1H), 1.49 (d, J=6.7 Hz, 3H). Anal. calcd. for C₇H₁₀Cl₂N₂Zn: Zn, 24.74; N, 10.83. Found: Zn, 25.19; N, 10.84.

### Example 33. Synthesis of zinc 2, 2′-bipyridine complex chloride (Zn-33)

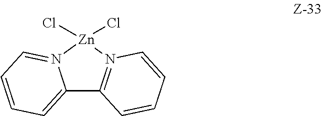

The procedure is the same as that of Example 1, starting from zinc chloride (4.0 g, 29.35 mmol) and 2, 2′-bipyridine (4.6 g, 29.35 mmol) to provide 6.5 g of zinc 2, 2′-bipyridine complex chloride complex as an off-white solid, 75.2% yield. 1H NMR (400 MHz, DMSO-d6) δ 8.77-8.54 (m, 4H), 8.22 (t, J=7.9 Hz, 2H), 7.76-7.63 (m, 2H). Anal. calcd. for C₁oHsCl₂N₂Zn: Zn, 21.86; N, 9.57. Found: Zn, 22.56; N, 9.58.

### Example 34. Synthesis of N, N-dimethylpiperidin-4-amine (1)

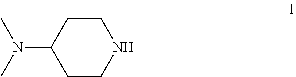

N-Boc piperidone (10 g, 0.05 mol) was dissolve in MeOH (100 mL), to which dimethylamine aqueous solution (25 mL, 0.22 mol) and 10% palladium on carbon (1 g) were added, and the reaction flask was evacuated and re-filled with hydrogen, then stirred at r.t. overnight. After filtration, the filtrate was concentrated and co-evaporated with dichloromethane for three times (3×80 mL), and dried on a vacuum pump to remove all dimethylamine. HCl/MeOH (4 M, 50 mL) was added to the residue and stirred at r.t. for 30 minutes. A large amount of white solid precipitated out and the mixture was filtered to yield a white solid 1(9 g, 90% yield). ESI-MS m/z: [M+H]⁺ calcd. for C₇H₁₆N₂, 129.13; found 129.13.

### Example 35. Synthesis of (9H-fluoren-9-yl)methyl 4-(dimethylamino)piperidine-1-carboxylate (2)

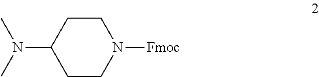

Compound 13 (2.0 g, 9.9 mmol) was dissolved in a mixed solution of 1, 4-dioxane and water (30 mL/50 mL), and sodium bicarbonate (2.5 g, 29.8 mmol) was added, and the mixture was cooled to 0° C. A solution of 9-fluorenylmethoxycarbonyl chloride (3.1 g, 11.9 mmol) in 1, 4-dioxane (10 mL) was added dropwise. After the addition, the temperature was gradually raised to r.t. and the reaction was stirred for 1 hour. 100 mL of 1M HCl was added, and the mixture was washed with ethyl acetate (3×50 mL), the aqueous phase was adjusted to pH˜10 with sodium carbonate, then extracted with dichloromethane (3×50 mL). The combined organic phases were washed with water (50 mL), dried over sodium sulfate, filtered, concentrated, and purified by column chromatography (MeOH/dichloromethane) to yield compound 2 (2.75 g, 79% yield). ESI-MS m/z: [M+H]⁺ calcd. for C₂₂H₂₆N₂O₂, 351.20; found 351.20.

### Example 36. Synthesis of (S)-tert-butyl (1-((4-(hydroxymethyl)phenyl)amino)-1-oxopropan-2-yl)carbamate (3)

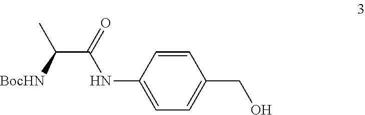 [SMILES: C[C@H](NC(=O)OC(C)(C)C)C(=O)Nc1ccc(CO)cc1]

p-aminobenzyl alcohol (5.0 g, 0.04 mol) and Boc-L-alanine (8.0 g, 0.042 mol) were dissolved in anhydrous THF (100 mL), and 2-ethoxy-1-ethoxycarbonyl-1, 2-dihydroquinoline (11 g, 0.044 mol) was added and stirred at r.t. overnight. The reaction mixture was poured into water (300 mL), extracted with ethyl acetate (3×100 mL), the combined organic phases were washed with water (100 mL), dried over sodium sulfate, filtered, and concentrated. The crude product was triturated with ethyl acetate/petroleum ether (1: 3) and filtered to yield compound 3 (9.8 g, 84% yield) as a white solid. ESI-MS m/z: [M+H]⁺ calcd. for C₁₅H₂₂N₂O₄: 295.16; found 295.16.

### Example 37. Synthesis of (S)-tert-butyl (1-((4-(bromomethyl)phenyl)amino)-1-oxopropan-2-yl)carbamate (4)

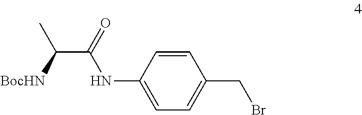 [SMILES: C[C@H](NC(=O)OC(C)(C)C)C(=O)Nc1ccc(CBr)cc1]

Compound 3 (3.5 g, 11.9 mmol) and carbon tetrabromide (5.9 g, 17.8 mmol) were dissolved in dichloromethane (80 mL), cooled to about 0° C., and triphenylphosphine (4.7 g, 17.8 mmol) was added. The reaction was warmed to r.t. and stirred for 30 minutes, and then 20 g of silica gel was added, mixed, and dried on a rotavap, loaded on a silica gel column (100 g of silica gel) and eluted with petroleum ether/ethyl acetate to yield compound 4 (2.6 g, 62% yield). ESI-MS m/z: [M+H]+ calcd. for C₁₅H₂₁BrN₂O₃: 357.07; found 357.07.

### Example 38. Synthesis of(S)-1-(((9H-fluoren-9-yl)methoxy)carbonyl)-N-(4-(2-((tert-butoxycarbonyl)amino)propanamido)benzyl)-N,_N-dimethylpiperidin-4-aminium bromide (5)

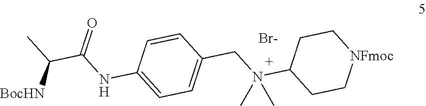

Compound 4 (2.3 g, 6.4 mmol) and compound 2 (2.7 g, 7.7 mmol) were dissolved in anhydrous THF (50 mL) and stirred at r.t. overnight. After removal of most THF on a rotavap, ethyl acetate (50 mL) was added to the residue. The resulting slurry was filtered to give a white solid (4.5 g, 100% yield). ESI-MS m/z: M calcd. for C₃₇H₄₇N₄O₅: 627.35; found 627.35.

### Example 39. Synthesis of (S)—N-(4-(2-((tert-butoxycarbonyl)amino)propanamido) benzyl)-N, N-dimethylpiperidin-4-aminium bromide (6)

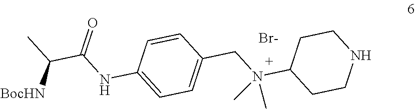

Compound 5 (1.0 g, 1.41 mmol) was dissolved in DMF (5 mL), and piperidine (1 mL) was added. After stirring at r.t. for 30 minutes, 30 mL of ethyl acetate was added and stirred for 10 minutes. The mixture was filtered to give a white powdery solid (550 mg, 80% yield). ESI-MS m/z: M⁺ calcd. for C₂₂H₃₇N₄O₃: 405.29; found 405.29.

### Example 40. Synthesis of N-(4-((S)-2-((tert-butoxycarbonyl)amino)propanamido) benzyl)-1-(((S)-4-ethyl-4, 9-dihydroxy-3, 14-dioxo-3, 4, 12, 14-tetrahydro-1H-pyrano[3′, 4′:6, 7]indolizino[1, 2-b]quinolin-10-yl)methyl)-N, N-dimethylpiperidin-4-aminium bromide (7)

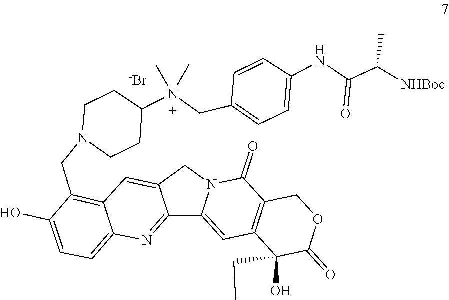

To a solution of 10-hydroxycamptothecin (375 mg, 1.03 mmol) in acetic acid (5 mL) was added a solution of compound 6 (550 mg, 1.13 mmol) and 37% formaldehyde (92 mg, 1.13 mmol) in acetic acid (5 mL). The mixture was heated to about 65° C. and stirred for 1 hour, then concentrated, co-evaporated with dry MeOH. Recrystallization in dichloromethane and a small amount of MeOH gave compound 7 (0.5 g, 63% yield) as a yellow powder. ESI-MS m/z: M⁺ calcd. for C₄₃H₅₃N₆O₈: 781.39; found 781.39.

### Example 41. Synthesis of N-(4-((S)-2-aminopropanamido)benzyl)-1-(((S)-4-ethyl-4, 9-dihydroxy-3, 14-dioxo-3, 4, 12, 14-tetrahydro-1H-pyrano[3′, 4′:6, 7]indolizino[1, 2-b]quinolin-10-yl)methyl)-N, N-dimethylpiperidin-4-aminium bromide (8)

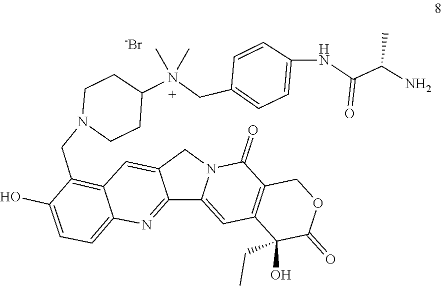

Compound 7 (50 mg, 0.058 mmol) was dissolved in a mixture of dichloromethane and trifluoroacetic acid (2 mL/6 mL), and stirred at r.t. for 30 minutes. The mixture was then concentrated and dried on a vacuum pump to give compound 8 (44 mg, 100% yield) as a yellow solid. ESI-MS m/z: M⁺ calcd. for C₃₈H₄₅N₆O₆: 681.34; found 681.34.

### Example 42. Synthesis of N-(4-((S)-2-(4-(2, 5-dioxo-2, 5-dihydro-1H-pyrrol-1-yl)butanamido)propanamido)benzyl)-1-(((S)-4-ethyl-4, 9-dihydroxy-3, 14-dioxo-3, 4, 12, 14-tetrahydro-1H-pyrano[3′, 4′:6, 7]indolizino[1, 2-b]quinolin-10-yl)methyl)-N, N-dimethylpiperidin-4-aminium formate (9)

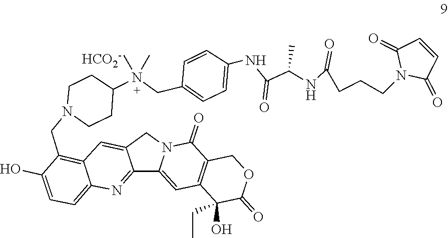

Compound 8 (88 mg, 0.116 mmol) and N-succinimidyl 4-maleimido-butyrate (49 mg, 0.140 mmol) were dissolved in DMF (5 mL), cooled to about 0° C., and then N, N-diisopropylethylamine (40 μL, 0.232 mmol) was added. The reaction was warmed to r.t. and stirred for 2 hours, concentrated, and purified by preparative HPLC (acetonitrile/water containing formic acid) to give compound 9 (66 mg, 68% yield). ESI-MS m/z: M calcd. for C₄₆H₅₂N₇O₉: 846.38; found 846.38.

### Example 43. Synthesis of 1-(tert-butyl) 5-(perfluorophenyl) ((benzyloxy)carbonyl)-L-glutamate (11)

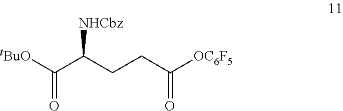

To a solution of Cbz-L-Glu-OtBu (135 g, 0.40 mol) in dichloromethane (2.0 L) was added pentafluorophenol (147 g, 0.80 mol) and DIC (202 g, 1.6 mol). The reaction was stirred at r.t. for 1 h, and then concentrated to give the crude title product (500 g).

### Example 44. Synthesis of tert-butyl (S)-30-(((benzyloxy)carbonyl)amino)-27-oxo-2,5,8,11,14,17,20,23-octaoxa-26-azahentriacontan-31-oate (12)

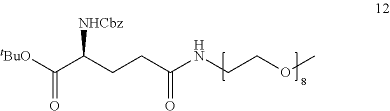

To the solution of mPEG₈-NH₂ (153 g, 0.4 mol) in DMF (2.5 L), DIPEA (206 g, 1.6 mol) and compound 11 (500 g, 0.4 mol, crude) were added and stirred at r.t. for 1 h. The resulting solution was concentrated and diluted with dichloromethane, washed with water. The aqueous layer was back-extracted with dichloromethane. The combined organic phase was washed with 0.2 N HCl and brine, dried over anhydrous Na₂SO₄, filtered and concentrated. Column chromatography (50% EtOAc/PE to pure EtOAc, then 10% methanol/dichloromethane) gave the title compound (260 g, 93% yield).

### Example 45. Synthesis of (S)-30-(((benzyloxy)carbonyl)amino)-27-oxo-2,5,8,11,14,17,20,23-octaoxa-26-azahentriacontan-31-oic acid (13)

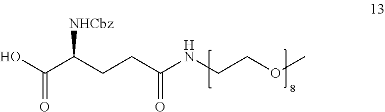

Compound 12 (255 g, 363 mmol) was dissolved in dichloromethane (500 mL) and HCOOH (1.0 L), and then stirred at room temperature overnight. The reaction mixture was diluted with chloromethane (3 L) and washed with water (1.5 L×3). The organic phase was concentrated and diluted with ethyl acetate (1.5 L), extracted with 5% NaHCO₃ solution (3 L). The aqueous layer was adjusted to pH 2˜3 using con. HCl, then extracted with dichloromethane, dried over sodium sulfate, filtered and concentrated to give the title compound (230 g, 98% yield).

### Example 46. Synthesis of perfluorophenyl (S)-30-(((benzyloxy)carbonyl)amino)-27-oxo-2,5,8,11,14,17,20,23-octaoxa-26-azahentriacontan-31-oate (14)

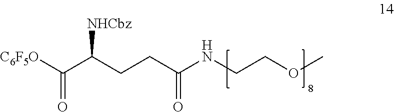

To a solution of compound 13 (220 g, 340 mmol) in dichloromethane (2.5 L) was added pentafluorophenol (125 g, 680 mmol) and DIC (171 g, 1.36 mol). The reaction was stirred at r.t. for 1 h, and then concentrated to give the crude title product (550 g).

### Example 47. Synthesis of tert-butyl (S)-30-(((benzyloxy)carbonyl)amino)-27,31-dioxo-2,5,8,11,14,17,20,23-octaoxa-26,32-diazahexatriacontan-36-oate (15)

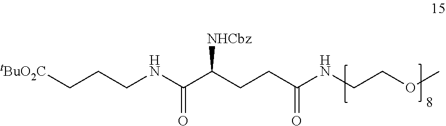

To a solution of tert-butyl 4-aminobutanoate (65.0 g, 410 mmol) in DMF (2.5 L) was added N, N-diisopropylethylamine (175 g, 1.36 mol). Compound 14 (550 g, 0.34 mol, crude) was then added at 10-20° C. and the resulting mixture was stirred at r.t. for 1 h. DMF was removed under vacuum and the residue was diluted with dichloromethane (2 L), washed with water twice, 0.2 N HCl and brine, dried over anhydrous Na₂SO₄, filtered and concentrated. Column chromatography (50% EtOAc/PE to pure EtOAc, then 0 to 5% methanol/dichloromethane) gave the title compound as a yellow oil (240 g, 90% yield).

### Example 48. Synthesis of tert-butyl (S)-30-amino-27,31-dioxo-2,5,8,11,14,17,20,23-octaoxa-26,32-diazahexatriacontan-36-oate (16)

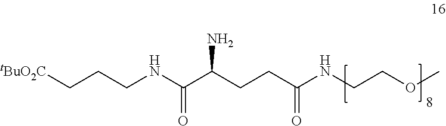

To a solution of compound 15 (220 g, 0.28 mol) in MeOH (1.0 L) was added Pd/C (20 g, 10% Pd/C, 50% wet). The mixture was hydrogenated under 1 atm H₂ at r.t. overnight, then filtered and concentrated to give the title compound (167 g, 91% yield).

### Example 49. Synthesis of tert-butyl (S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oate (17)

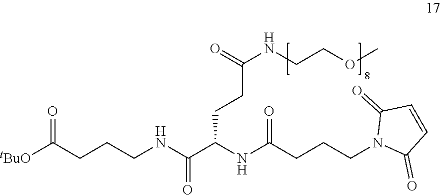

To a solution of compound 16 (167 g, 0.26 mmol) in DMF (1.0 L), DIPEA (132 g, 1.02 mol) and perfluorophenyl 4-(2,5-dioxo-2,5-dihydro-1H-pyrrol-1-yl)butanoate (250 g, 0.26 mol, crude) were added. The mixture was stirred for 1 h, then concentrated and diluted with dichloromethane (2.0 L) and washed with water twice, 0.2 N HCl and brine, dried over anhydrous sodium sulfate, filtered, concentrated and purified by silica gel column chromatography (50-100% ethyl acetate/petroleum ether and 0-10% methanol/dichloromethane) to give the title compound as a light yellow oil (201 g, 94% yield).

### Example 50. Synthesis of(S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oic acid (18)

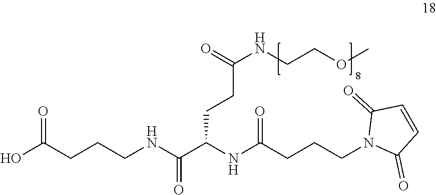

Compound 17 (16.8 g, 20.5 mmol) was dissolved in dichloromethane (60 mL) and HCOOH (120 mL), and then stirred at room temperature overnight. The reaction mixture was concentrated and extracted with ethyl acetate (150 mL). NaCl was added to the aqueous phase until saturation and the solution was extracted with dichloromethane (200 mL×2). The organic phase was dried over sodium sulfate, filtered and concentrated, purified by column chromatography (0 to 20% methanol/dichloromethane) to give the title compound (16.4 g, crude product containing formic acid). ESI MS m/z: [M+H]⁺ calcd. for C₃₄H₅₉O₁₅N₄ 763.39; found 763.29.

### Example 51. Synthesis of 2,5-dioxopyrrolidin-1-yl (S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oate (19)

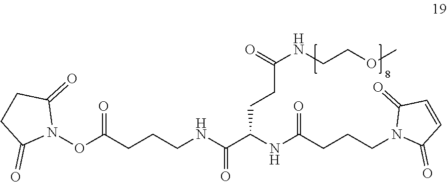

To a solution of compound 18 (15.6 g, 20.5 mol) in dichloromethane (200 mL), NHS (3.7 g, 32.3 mmol) and EDC-HCl (8.3 g, 43.0 mmol) were added, and the reaction was stirred at r.t. for 30 min, then washed with brine, dried over anhydrous sodium sulfate, filtered and concentrated to give a colorless oil compound (17.6 g, 100% yield). ESI MS m/z: [M+H]⁺ calcd. for C₃₈H₆₂O₁₇N₅ 860.41; found 860.29.

### Example 52. Synthesis of N-(4-((9S, 17S)-9-(4-(2, 5-dioxo-2, 5-dihydro-1H-pyrrol-1-yl)butanamido)-17-methyl-6, 10, 15-trioxo-2-oxa-5, 11, 16-triazaoctadecanamido)benzyl)-1-(((S)-4-ethyl-4, 9-dihydroxy-3, 14-dioxo-3, 4, 12, 14-tetrahydro-1H-pyrano[3′, 4′:6, 7]indolizino[1, 2-b]quinolin-10-yl)methyl)-N, N-dimethylpiperidin-4-aminium formate (20)

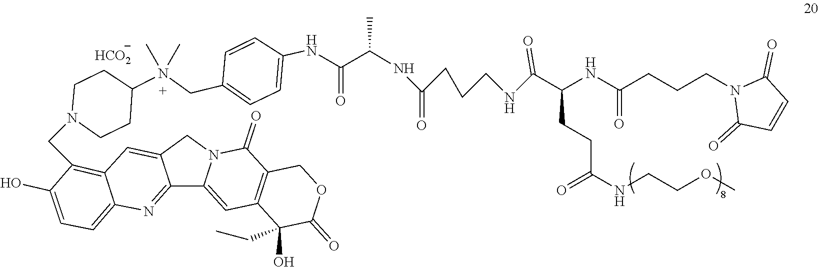

Compound 8 (44 mg, 0.058 mmol) and compound 19 (60 mg, 0.065 mmol) were dissolved in DMF (5 mL), cooled to about 0° C., and then N, N-diisopropylethylamine (20 μL, 0.116 mmol) was added. The reaction was warmed to r.t. and stirred for 2 hours, concentrated, and purified by preparative HPLC (acetonitrile/water containing formic acid) to give compound 20 (51 mg, 58% yield). ESI-MS m/z: M⁺ calcd. for C₇₂H₁₀₁N₁₀O₂₀: 1425.72; found 1425.72.

### Example 53. Synthesis of 1-(2-amino-4-fluoro-5-methoxyphenyl)-2-chloroethanone (21)

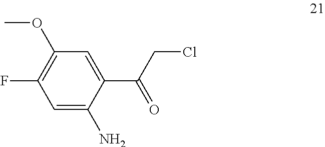

A solution of 3-fluoro-4-methoxyaniline (5 g, 35.4 mmol) dissolved in dichloromethane (20 mL) was added dropwise to an ice water cooled boron trichloride (1 M in dichloromethane, 38.9 mL) solution. The reaction was stirred for 10 minutes and then chloroacetonitrile (3.2 g, 42.5 mmol) and aluminum trichloride (5.2 g, 38.9 mmol) were added. After the addition was completed, the reaction was warmed to r.t. and then refluxed overnight. The reaction mixture was then cooled to about 0° C., quenched with 2 M HCl (80 mL) and stirred at r.t. for 2 hours. Layers were separated and the aqueous phase was extracted with dichloromethane (3×80 mL). Combined organic phases were washed with water (100 mL), dried over sodium sulfate, filtered, concentrated, purified on a silica gel column, eluted with petroleum ether/ethyl acetate to give compound 21 (2 g, 26% yield) as a yellow solid. ESI-MS m/z: [M+H]⁺ calcd. for C₉H9ClFNO₂: 218.03; found 218.03.

### Example 54. Synthesis of (S)-11-(chloromethyl)-4-ethyl-8-fluoro-4-hydroxy-9-methoxy-1H-pyrano[3′, 4′:6, 7]indolizino[1, 2-b]quinoline-3, 14(4H, 12H)-dione (22)

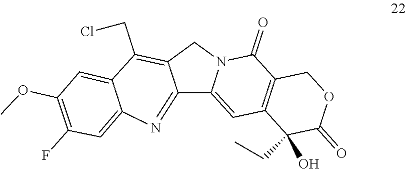 [SMILES: CC[C@@]1(O)C(=O)OCc2c1cc1n(c2=O)Cc2c-1nc1cc(F)c(OC)cc1c2CCl]

Compound 21 (0.50 g, 2.29 mmol) and (S)-4-ethyl-4-hydroxy-7,8-dihydro-1H-pyrano[3,4-f]indolizine-3,6,10(4H)-trione (0.57 g, 2.19 mmol) were dissolved in anhydrous toluene (40 mL), and p-toluenesulfonic acid (42 mg, 0.219 mmol) was added. The suspension was heated at reflux for 2 days and allowed to cool to r.t. After removal of about two-thirds of toluene, the residue was filtered and the filter cake was washed with dichloromethane, air-dried to give compound 22 (0.7 g, 72% yield) as a gray powdery solid. ESI-MS m/z: [M+H]⁺ calcd. for C₂₂H₁₈C₁FN₂O₅: 445.09; found 445.09.

### Example 55. Synthesis of N-(4-((S)-2-((tert-butoxycarbonyl)amino)propanamido) benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium chloride (23)

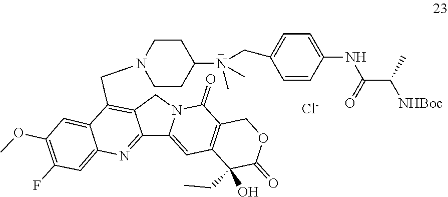

A mixture of compound 22 (218 mg, 0.49 mmol), compound 6 (200 mg, 0.49 mmol) in DMF (5 mL) was stirred at 0° C. for 30 minutes, then triethylamine (63 μL, 0.45 mmol) was added and the stirring was continued for 1 hour. The reaction was concentrated and purification by preparative HPLC (acetonitrile/water containing formic acid) gave compound 23 (240 mg, 59% yield) as a yellow solid. ESI-MS m/z: M⁺ calcd. for C₄₄H₅₄FN₆O₈: 813.40; found 813.40.

### Example 56. Synthesis of N-(4-((S)-2-aminopropanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium (24)

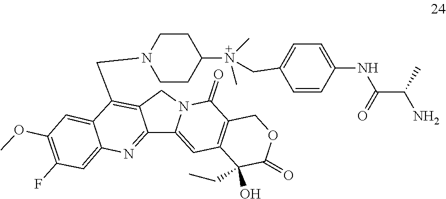

Compound 23 (50 mg, 0.06 mmol) was dissolved in a mixture of dichloromethane and trifluoroacetic acid (2 mL/6 mL), and stirred at r.t. for 30 minutes. The mixture was then concentrated and dried on a vacuum pump to give compound 24 (42 mg, 100% yield) as a yellow solid. ESI-MS m/z: M⁺ calcd. for C₃₉H₄₆FN₆06: 713.35; found 713.35.

### Example 57. Synthesis of N-(4-((30S, 38S)-30-(4-(2, 5-dioxo-2, 5-dihydro-1H-pyrrol-1-yl)butanamido)-38-methyl-27, 31, 36-trioxo-2, 5, 8, 11, 14, 17, 20, 23-octaoxa-26, 32, 37-triazanonatriacontanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium formate (25)

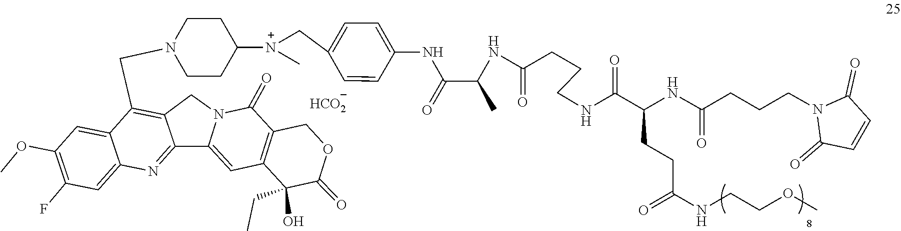

Compound 24 (47 mg, 0.060 mmol) and compound 19 (60 mg, 0.066 mmol) were dissolved in DMF (5 mL), cooled to about 0° C., and then N, N-diisopropylethylamine (21 μL, 0.12 mmol) was added. The reaction was warmed to r.t. and stirred for 2 hours, concentrated, and purified by preparative HPLC (acetonitrile/water containing formic acid) (acetonitrile/water containing formic acid) to give compound 25 (23 mg, 25% yield). ESI-MS m/z: M⁺ calcd. for C₇₃H₁₀₂FN₁₀O₂₀: 1457.73; found 1457.73.

### Example 58. Synthesis of (S)-11-(aminomethyl)-4-ethyl-8-fluoro-4-hydroxy-9-methoxy-1H-pyrano[3′, 4′:6, 7]indolizino[1, 2-b]quinoline-3, 14(4H, 12H)-dione (26)

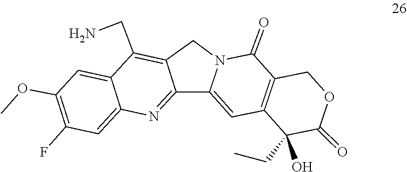 [SMILES: CC[C@@]1(O)C(=O)OCc2c1cc1n(c2=O)Cc2c-1nc1cc(F)c(OC)cc1c2CN]

Compound 22 (80 mg, 0.18 mmol) was dissolved in ethanol (5 mL), hexamethylenetetramine (76 mg, 0.54 mmol) was added and the mixture was refluxed for 90 minutes and then cooled to r.t. Concentrated hydrochloric acid (100 L) was added, and stirred for 30 minutes. After concentration, an off-white solid was obtained, which was purified by preparative HPLC (acetonitrile/water containing formic acid) to give compound 26 (40 mg, 52% yield). ESI-MS m/z: [M+H]⁺ calcd. for C₂₂H₂₀FN₃O₅: 426.14; found 426.14.

### Example 59. Synthesis of (S)-2-(4-(2, 5-dioxo-2, 5-dihydro-1H-pyrrol-1-yl)butanamido)-N₁-(4-((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-10-yl)methyl)amino)-4-oxobutyl)-N₅-(2, 5, 8, 11, 14, 17, 20, 23-octaoxapentacosan-25-yl)pentanediamide (27)

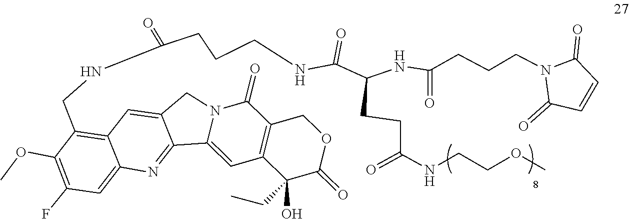

Compound 26 (40 mg, 0.094 mmol) and compound 19 (120 mg, 0.13 mmol) were dissolved in DMF (5 mL), cooled to about 0° C., and then N, N-diisopropylethylamine (33 μL, 0.188 mmol) was added. The reaction was warmed to r.t. and stirred for 2 hours, concentrated, and purified by preparative HPLC (acetonitrile/water containing formic acid) to give compound 27 (55 mg, 50% yield). ESI-MS m/z: [M+H]⁺ calcd. for C₅₆H₇₆FN₇O₁₉: 1170.52; found 1170.52.

### Example 60. Synthesis of tert-butyl (1-methylpiperidin-4-yl)carbamate (28)

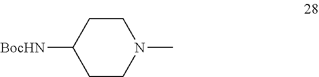

4-(tert-butoxycarbonylamino)piperidine (2 g, 10 mmol) was dissolved in MeOH (30 mL), and then 37% formaldehyde (1.6 g, 20 mmol) and 10% palladium on carbon (0.2 g) were added. The reaction was stirred under 1 atm hydrogen overnight and filtered. The filtrate was concentrated to give compound 28 (2.1 g, 100% yield). ESI-MS m/z: [M+H]⁺ calcd. for C₁₁H₂₂N₂O₂: 215.17; found 215.17.

### Example 61. Synthesis of (S)-4-((tert-butoxycarbonyl)amino)-1-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperidin-1-ium chloride (29)

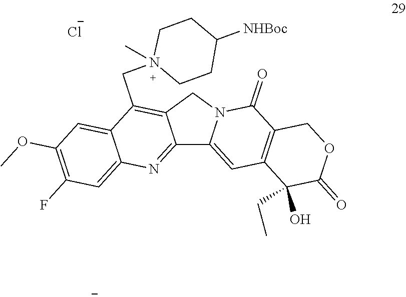

Compound 22 (50 mg, 0.112 mmol) and compound 28 (26 mg, 0.123 mmol) in DMF (3 mL) was stirred at r.t. for 2 hours. The reaction solution was purified by preparative HPLC (acetonitrile/water containing formic acid) to give compound 29 (33 mg, 47% yield). ESI-MS m/z: [M]⁺ calcd. for C₃₃H₄₀FN₄O₇: 623.29; found 623.29.

### Example 62. Synthesis of (S)-4-amino-1-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperidin-1-ium (30)

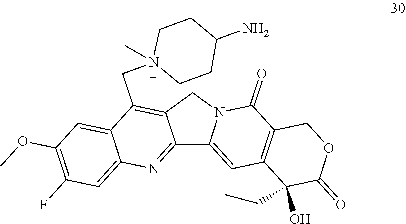

Compound 29 (30 mg, 0.053 mmol) was dissolved in a mixture of dichloromethane and trifluoroacetic acid (3 mL/1 mL), and stirred at r.t. for 30 minutes. The mixture was then concentrated and dried on a vacuum pump to give compound 30 (33 mg, 100% yield). ESI-MS m/z: [M]⁺ calcd. for C₂₈H₃₂N₄O₅: 477.21; found 477.21.

### Example 63. Synthesis of 4-((S)-30-(4-(2, 5-dioxo-2, 5-dihydro-1H-pyrrol-1-yl)butanamido)-27, 31-dioxo-2, 5, 8, 11, 14, 17, 20, 23-octaoxa-26, 32-diazahexa triacontanamido)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methyl piperidin-1-ium formate (31)

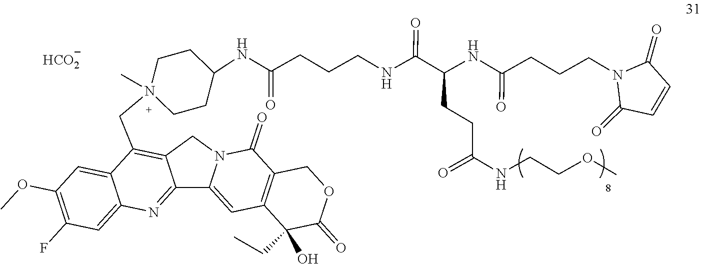

Compound 30 (30 mg, 0.053 mmol) and compound 19 (60 mg, 0.079 mmol) were dissolved in DMF (5 mL), cooled to about 0° C., and then N, N-diisopropylethylamine (18 μL, 0.106 mmol) was added. The reaction was warmed to r.t. and stirred for 2 hours, concentrated, and purified by preparative HPLC (acetonitrile/water containing formic acid) to give compound 31 (15 mg, 21% yield). ESI-MS m/z: [M]⁺ calcd. for C₆₂H₈₈FN₈O₁₉: 1267.61; found 1267.61.

### Example 64. Synthesis of (9H-fluoren-9-yl)methyl 4-methylpiperazine-1-carboxylate (32)

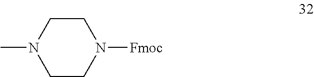

1-methylpiperazine (5.0 g, 50.0 mmol) was dissolved in a mixed solution of 1, 4-dioxane and water (60 mL/100 mL), and sodium bicarbonate (12.6 g, 150 mmol) was added, and the mixture was cooled to 0° C. A solution of 9-fluorenylmethoxycarbonyl chloride (15.5 g, 60.0 mmol) in 1, 4-dioxane (20 mL) was added dropwise. After the addition, the temperature was gradually raised to r.t. and the reaction was stirred for 3 hours. 300 mL of 1M HCl was added, and the mixture was washed with ethyl acetate (2×100 mL), the aqueous phase was adjusted to pH˜10 with sodium carbonate, then extracted with ethyl acetate (2×100 mL). The combined organic phases were washed with water (250 mL), dried over sodium sulfate, filtered, concentrated, and purified by column chromatography (MeOH/dichloromethane) to yield compound 32 (6.5 g, 40% yield). ESI-MS m/z: [M+H]⁺ calcd. for C₂₀H₂₂N₂O₂, 323.17; found 323.19.

### Example 65. Synthesis of(S)-4-(((9H-fluoren-9-yl)methoxy)carbonyl)-1-(4-(2-((tert-butoxycarbonyl)amino)propanamido)benzyl)-1-methylpiperazin-1-ium bromide (33)

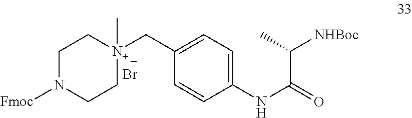

Compound 4 (2.3 g, 6.4 mmol) and compound 32 (2.1 g, 6.4 mmol) were dissolved in anhydrous THF (100 mL) and stirred at r.t. overnight. After removal of most THF on a rotavap, ethyl acetate (200 mL) was added to the residue. The resulting slurry was filtered to give a white solid (3.8 g, 87% yield). ESI-MS m/z: M⁺ calcd. for C₃₅H₄₃N₄O₅: 599.32; found 599.32.

### Example 66. Synthesis of (S)-1-(4-(2-((tert-butoxycarbonyl)amino)propanamido) benzyl)-1-methylpiperazin-1-ium bromide (34)

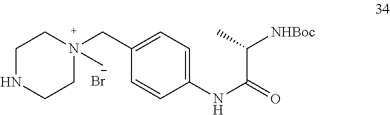

Compound 33 (3.12 g, 4.6 mmol) was dissolved in DMF (25 mL), and piperidine (3 mL) was added. After stirring at r.t. for 2 hours, 200 mL of ethyl acetate was added and stirred for 10 minutes. The mixture was filtered to give a white solid (1.54 g, 77% yield). ESI-MS m/z: M⁺ calcd. for C₂₀H₃₃N₄O₃: 377.26; found 377.26.

### Example 67. Synthesis of 1-(4-((S)-2-((tert-butoxycarbonyl)amino)propanamido) benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (35)

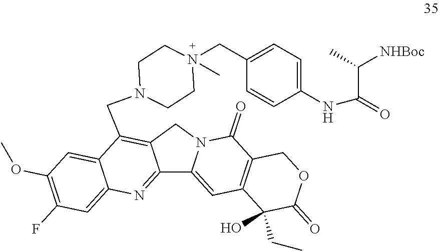

A mixture of compound 34 (0.30 g, 0.66 mmol), compound 22 (0.25 g, 0.56 mmol) in DMF (10 mL) was stirred at 0° C. for 30 minutes, then N, N-diisopropylethylamine (49 μL, 0.28 mmol) was added and the reaction was warmed to r.t. and stirred overnight, concentrated and purification by preparative HPLC (acetonitrile/water containing formic acid) to give compound 35 (0.40 g, 80% yield). ESI-MS m/z: M⁺ calcd. for C₄₂H₅₀FN₆O₈: 785.37; found 785.37.

### Example 68. Synthesis of 1-(4-((S)-2-aminopropanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (36)

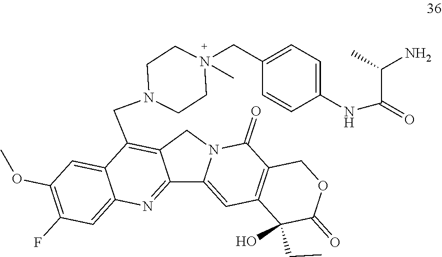

Compound 35 (0.30 g, 0.35 mmol) was dissolved in a mixture of dichloromethane and trifluoroacetic acid (3 mL/3 mL), and stirred at r.t. for 30 minutes. The mixture was then concentrated and dried on a vacuum pump to give compound 36 (0.27 g, 100% yield) as a yellow solid. ESI-MS m/z: M⁺ calcd. for C₃₇H₄₂FN₆06: 477.21; found 477.21.

### Example 69. Synthesis of 1-(4-((S)-2-(4-(2, 5-dioxo-2, 5-dihydro-1H-pyrrol-1-yl)butanamido)propanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium formate (37)

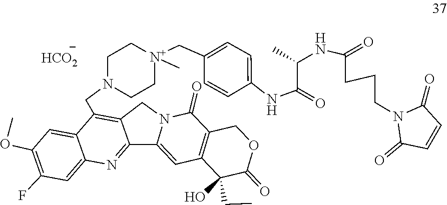 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)CCCN3C(=O)C=CC3=O)cc2)CC1 ; "HCO2"]

Compound 36 (50 mg, 0.065 mmol) and N-succinimidyl 4-maleimido-butyrate (30 mg, 0.098 mmol) were dissolved in DMF (3 mL), and then N, N-diisopropylethylamine (45 μL, 0.26 mmol) was added. The reaction was stirred at r.t for 30 minutes, concentrated, and purified by preparative C-18 HPLC (acetonitrile/water containing formic acid) to give compound 37 (37 mg, 61% yield). ESI-MS m/z: M⁺ calcd. for C₄₅H₄₉FN₇O₉: 850.36; found 850.36.

### Example 70. Synthesis of 1-(4-((30S, 38S)-30-(4-(2, 5-dioxo-2, 5-dihydro-1H-pyrrol-1-yl)butanamido)-38-methyl-27, 31, 36-trioxo-2, 5, 8, 11, 14, 17, 20, 23-octaoxa-26, 32, 37-triazanonatriacontanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium formate (38)

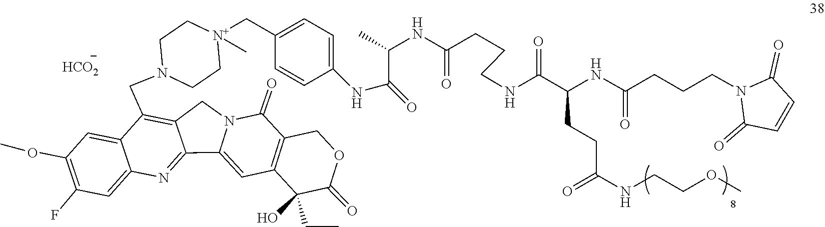

Compound 36 (70 mg, 0.092 mmol) was dissolved in DMF (2 mL), to which compound 18 (70 mg, 0.092 mmol) in DMF (2 mL) was added, followed by HATU (52 mg, 0.138 mmol) and triethylamine (52 μL, 0.368 mmol) in sequence, and the reaction was stirred at r.t. for 30 minutes. After concentration, the residue was purified by preparative HPLC (acetonitrile/water containing formic acid) to give compound 38 (50.9 mg, 37% yield). ESI-MS m/z: [M]⁺ calcd. for C₇₁H₉₈FN₁₀O₂₀: 1429.69; found 1429.69.

### Example 71. Synthesis of 1-(4-((S)-17-(2, 5-dioxo-2, 5-dihydro-1H-pyrrol-1-yl)-2-methyl-4, 14-dioxo-7, 10-dioxa-3, 13-diazaheptadecanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium formate (39)

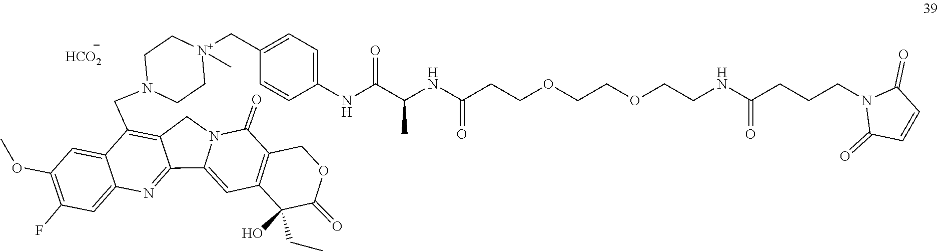 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)CCOCCOCCNC(=O)CCCN3C(=O)C=CC3=O)cc2)CC1 ; "HCO2"]

Compound 36 (0.10 g, 0.13 mmol) in DMF (1 mL) and 2,5-dioxopyrrolidin-1-yl 3-(2-(2-(4-(2,5-dioxo-2,5-dihydro-1H-pyrrol-1-yl)butanamido)ethoxy)ethoxy)propanoate (57 mg, 0.13 mmol) in DMF (2 mL) were mixed, and then N, N-diisopropylethylamine (90 μL, 0.52 mmol) was added. The reaction mixture was stirred at r.t. for 1 hour, concentrated, and purified by preparative HPLC (acetonitrile/water containing formic acid) to give compound 39 (50.9 mg, 39% yield). ESI-MS m/z: M⁺ calcd. for C₅₂H₆₂N₈O₁₂: 1009.45; found 1009.45.

### Example 72. Synthesis of (S)-3-((tert-butoxycarbonyl)amino)-2-(2, 5-dioxo-2, 5-dihydro-1H-pyrrol-1-yl)propanoic acid (40)

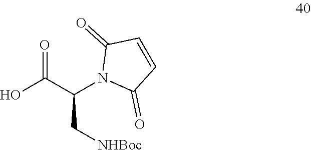 [SMILES: CC(C)(C)OC(=O)NC[C@@H](C(=O)O)N1C(=O)C=CC1=O]

To the solution of 2-amino-3-((tert-butoxycarbonyl)amino)propanoic acid (1 g, 4.90 mmol) in a saturated solution of NaHCO₃ (20 mL) was added methyl 2, 5-dioxo-2, 5-dihydro-1H-pyrrole-1-carboxylate (1.52 g, 9.80 mmol) in ice-water bath. The reaction was stirred for 30 min and then poured into a separatory funnel containing 100 mL of ethyl acetate and the organic phase was separated, washed with 50 mL of water and 50 mL of brine, dried over anhydrous Na₂SO₄, filtered and concentrated to give compound 40 (1.39 g, yield 72%).

### Example 73. Synthesis of (S)-perfluorophenyl 3-((tert-butoxycarbonyl)amino)-2-(2, 5-dioxo-2, 5-dihydro-1H-pyrrol-1-yl)propanoate (41)

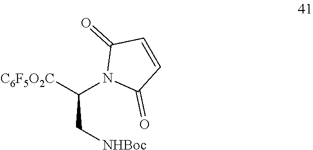

To a solution of compound 40 (0.10 g, 0.35 mmol) dissolved in dichloromethane (30 mL), were added pentafluorophenol (97 mg, 0.52 mmol) and 1-(3-dimethylaminopropyl)-3-ethylcarbodiimide hydrochloride (0.13 g, 0.7 mmol). The reaction was stirred at r.t. for 2 hours and diluted with dichloromethane (50 mL), washed with water (200 mL), dried over sodium sulfate, filtered, and concentrated to give compound 41 (0.16 g, 100% yield). ESI-MS m/z: [M+H]⁺ calcd. for C₁₈H₁₅F₅N₂O₆: 451.09; found 451.09.

### Example 74. Synthesis of Compound 42

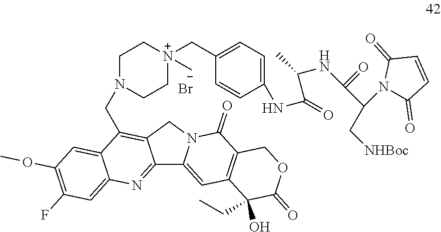

Compound 36 (0.05 g, 0.065 mmol) and compound 41 (45 mg, 0.10 mmol) were dissolved in DMF (3 mL), and then N, N-diisopropylethylamine (45 μL, 0.26 mmol) was added. The reaction was stirred at r.t. for 1 hour, concentrated and purified by preparative HPLC (acetonitrile/water containing formic acid) to yield compound 42 (35 mg, 52% yield). ESI-MS m/z: M⁺ calcd. for C₄₉H₅₆FN₈O₁₁: 951.41; found 951.41.

### Example 75. Synthesis of 1-(4-((S)-2-((S)-3-amino-2-(2, 5-dioxo-2, 5-dihydro-1H-pyrrol-1-yl)propanamido)propanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (43)

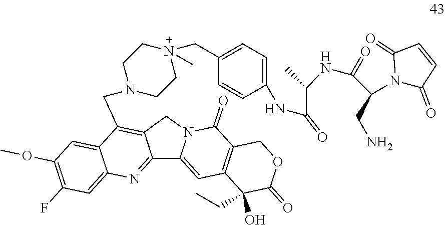 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@H](CN)N3C(=O)C=CC3=O)cc2)CC1]

Compound 42 (35 mg, 0.03 mmol) was dissolved in dichloromethane (2 mL) and treated with trifluoroacetic acid (1 mL). After stirring at r.t. for 1 hour, the reaction mixture was concentrated, co-evaporated with dichloromethane twice and dried on a vacuum pump to give compound 43 (30.4 mg, 96% yield). ESI-MS m/z: M calcd. for C₄₄H₄₈FN₈O₉: 851.35; found 851.35.

### Example 76. Synthesis of (S)-tert-butyl (1-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)carbamate (44)

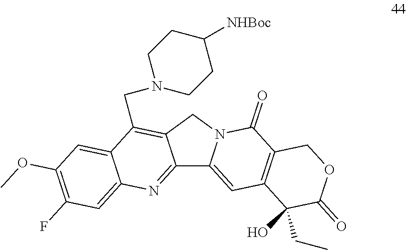 [SMILES: CC[C@@]1(O)C(=O)OCc2c1cc1n(c2=O)Cc2c-1nc1cc(F)c(OC)cc1c2CN1CCC(NC(=O)OC(C)(C)C)CC1]

Compound 22 (50 mg, 0.11 mmol) was dissolved in DMF (3 mL), and then tert-butyl piperidin-4-ylcarbamate (25 mg, 0.12 mmol) was added and stirred at r.t. for 5 hours. The mixture was concentrated and purified by preparative HPLC (acetonitrile/water containing formic acid) to yield compound 44 (30 mg, 45% yield). ESI-MS m/z: [M+H]⁺ calcd. for C₃₂H₃₇FN₄O₇: 609.26; found 609.26.

### Example 77. Synthesis of (S)-11-((4-aminopiperidin-1-yl)methyl)-4-ethyl-8-fluoro-4-hydroxy-9-methoxy-1H-pyrano[3′, 4′:6, 7]indolizino[1, 2-b]quinoline-3, 14(4H, 12H)-dione (45)

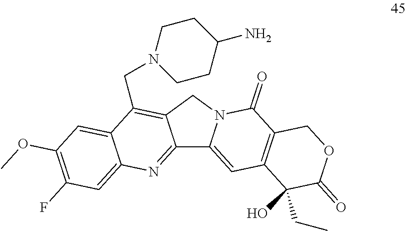 [SMILES: CC[C@@]1(O)C(=O)OCc2c1cc1n(c2=O)Cc2c-1nc1cc(F)c(OC)cc1c2CN1CCC(N)CC1]

Compound 44 (30 mg, 0.03 mmol) was dissolved in dichloromethane (2 mL) and treated with trifluoroacetic acid (2 mL). After stirring at r.t. for 1 hour, the mixture was concentrated, co-evaporated with dichloromethane twice and dried on a vacuum pump to give compound 45 (25.4 mg, 100% yield). ESI-MS m/z: [M+H]⁺ calcd. for C₂₇H₃₀FN₄₅: 509.21; found 509.21.

### Example 78. Synthesis of (S)-2-(4-(2, 5-dioxo-2, 5-dihydro-1H-pyrrol-1-yl)butanamido)-N₁-(4-((1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)amino)-4-oxobutyl)-N₅-(2, 5, 8, 11, 14, 17, 20, 23-octaoxapentacosan-25-yl)pentanediamide (46)

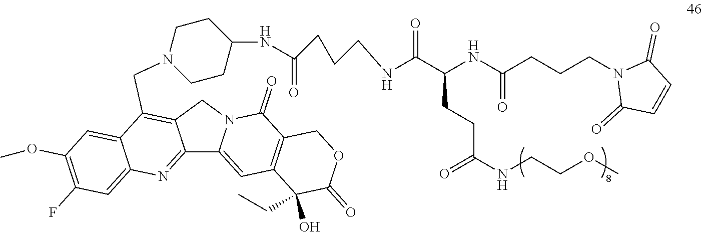

Compound 45 (25.4 mg, 0.05 mmol) was dissolved in DMF (2 mL), to which compound 19 (38.1 mg, 0.05 mmol) was added, followed by HATU (28.5 mg, 0.08 mmol) and triethylamine (14 μL, 0.1 mmol) in sequence, and the reaction was stirred at r.t. for 1 h, concentrated and purified by preparative HPLC (acetonitrile/water containing formic acid) to give compound 46 (14.4 mg, 23% yield). ESI-MS m/z: [M+H]⁺ calcd. for C₆₁H₈₅FN₈O₁₉: 1253.59; found 1253.59.

### Example 79. Synthesis of tert-butyl bis(2-(2, 2, 2-trifluoroacetamido)ethyl)carbamate (47)

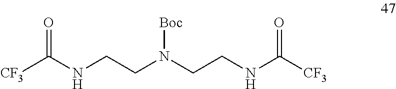 [SMILES: CC(C)(C)OC(=O)N(CCNC(=O)C(F)(F)F)CCNC(=O)C(F)(F)F]

To a solution of diethylenetriamine (6.18 g, 60 mmol) in dichloromethane (120 mL), was added dropwise a solution of ethyl trifluoroacetate (18.75 g, 132 mmol) in dichloromethane (60 mL) at 0° C. The solution was stirred for 30 minutes, and then warmed to r.t. and stirred for 1 hour. A solution of di-tert-butyl dicarbonate (28.78 g, 132 mmol) and triethylamine (13.33 g, 132 mmol) in dichloromethane (60 mL) was added dropwise at r.t. and stirred overnight. The reaction mixture was washed with saturated sodium carbonate (2×200 mL), water (2×200 mL), brine (200 mL), dried over sodium sulfate, filtered and concentrated. The residue was purified by silica gel column (petroleum ether/ethyl acetate) to give a white solid (17.4 g, 73.3% yield). ESI-MS m/z: [M+H]⁺ calcd. for C₁₃H₁₉F₆N₃O₄: 396.30; found 396.28.

### Example 80. Synthesis of tert-butyl bis(2-aminoethyl)carbamate (48)

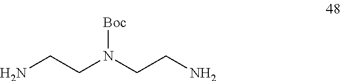 [SMILES: CC(C)(C)OC(=O)N(CCN)CCN]

Compound 47 (4.28 g, 10.8 mmol) was dissolved in MeOH (50 mL) and stirred with a solution of sodium hydroxide (5.42 g, 135 mmol) in water (50 mL) at r.t. for 3 hours. The reaction was concentrated, extracted with dichloromethane (3×100 mL), the organic phase was washed with brine (100 mL), dried with sodium sulfate, filtered and concentrated to give compound 3 (1.8 g, 82% yield). ESI-MS m/z: [M+H]⁺ calcd. for C₉H₂₁N₃O₂ 204.28; found 204.12.

### Example 81. Synthesis of 4, 4′-((((tert-butoxycarbonyl)azanediyl)bis(ethane-2, 1-diyl))bis(azanediyl))bis(4-oxobutanoic acid) (49)

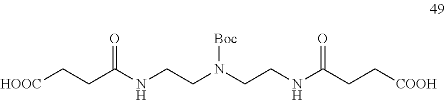 [SMILES: CC(C)(C)OC(=O)N(CCNC(=O)CCC(=O)O)CCNC(=O)CCC(=O)O]

Compound 48 (1.8 g, 8.8 mmol) was dissolved in dichloromethane (150 mL), to which succinic anhydride (2.2 g, 22.1 mmol) was added. After stirring at r.t. overnight, the reaction was concentrated and purified on silica gel column, eluting with dichloromethane/MeOH to yield compound 49 (2.99 g, 84% yield). ESI-MS m/z: [M+H]⁺ calcd. for C₁₇H₂₉N₃O₈: 404.43; found 404.11.

### Example 82. Synthesis of bis((S)-4-ethyl-4-hydroxy-3, 14-dioxo-3, 4, 12, 14-tetrahydro-1H-pyrano[3′, 4′:6, 7]indolizino[1, 2-b]quinolin-9-yl) 4, 4′-((((tert-butoxycarbonyl)azanediyl)bis(ethane-2, 1-diyl))bis(azanediyl))bis(4-oxobutanoate) (50)

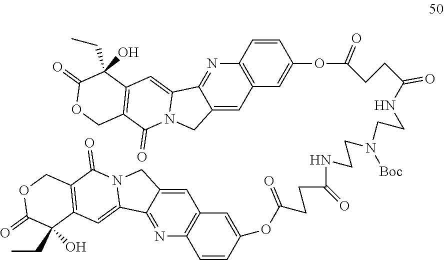 [SMILES: CC[C@@]1(O)C(=O)OCc2c1cc1n(c2=O)Cc2cc3cc(OC(=O)CCC(=O)NCCN(CCNC(=O)CCC(=O)Oc4ccc5nc6c(cc5c4)Cn4c-6cc5c(c4=O)COC(=O)[C@]5(O)CC)C(=O)OC(C)(C)C)ccc3nc2-1]

To a solution of compound 49 (853 mg, 2.1 mmol) and (S)-4-ethyl-4,9-dihydroxy-1,12-dihydro-14H-pyrano[3′,4′:6,7]indolizino[1,2-b]quinoline-3,14(4H)-dione (1.71 g, 4.7 mmol) in DMF (100 mL), triethylamine (948 mg, 9.4 mmol) and HATU (1.79 g, 4.7 mmol) were added in sequence. The resulting mixture was stirred overnight, and then concentrated, purified by silica gel column (dichloromethane/MeOH) to give compound 50 (2.84 g, 100% yield). ESI-MS m/z: [M+H]⁺ calcd. for C₅₇H₅₇N₇O₁₆: 1097.10; found 1097.65.

### Example 83. Synthesis of bis((S)-4-ethyl-4-hydroxy-3, 14-dioxo-3, 4, 12, 14-tetrahydro-1H-pyrano[3′, 4′:6, 7]indolizino[1, 2-b]quinolin-9-yl) 4, 4′-((azanediylbis(ethane-2, 1-diyl))bis(azanediyl))bis(4-oxobutanoate) (51)

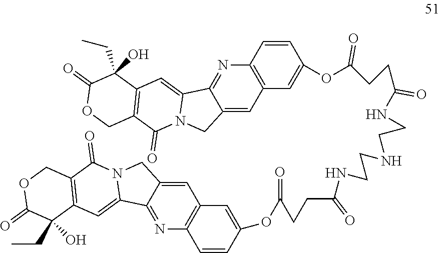 [SMILES: CC[C@@]1(O)C(=O)OCc2c1cc1n(c2=O)Cc2cc3cc(OC(=O)CCC(=O)NCCNCCNC(=O)CCC(=O)Oc4ccc5nc6c(cc5c4)Cn4c-6cc5c(c4=O)COC(=O)[C@]5(O)CC)ccc3nc2-1]

Compound 50 (2.84 g, 2.1 mmol) was dissolved in dichloromethane (40 mL). and trifluoroacetic acid (20 mL) was added. The reaction was stirred at r.t. for 1 hour and then concentrated to give compound 51 (3.3 g, 100% yield). ESI-MS m/z: [M+H]⁺ calcd. for C₅₂H₄₉N₇O₁₄: 996.98; found 996.60.

### Example 84. Synthesis of (S)—(S)-4-ethyl-4-hydroxy-3, 14-dioxo-3, 4, 12, 14-tetrahydro-1H-pyrano[3′, 4′:6, 7]indolizino[1, 2-b]quinolin-9-yl 30-(4-(2, 5-dioxo-2, 5-dihydro-1H-pyrrol-1-yl)butanamido)-37-(2-(4-(((S)-4-ethyl-4-hydroxy-3, 14-dioxo-3, 4, 12, 14-tetrahydro-1H-pyrano[3′, 4′:6, 7]indolizino[1, 2-b]quinolin-9-yl)oxy)-4-oxobutanamido)ethyl)-27, 31, 36, 41-tetraoxo-2, 5, 8, 11, 14, 17, 20, 23-octaoxa-26, 32, 37, 40-tetraazatetratetracontan-44-oate (52)

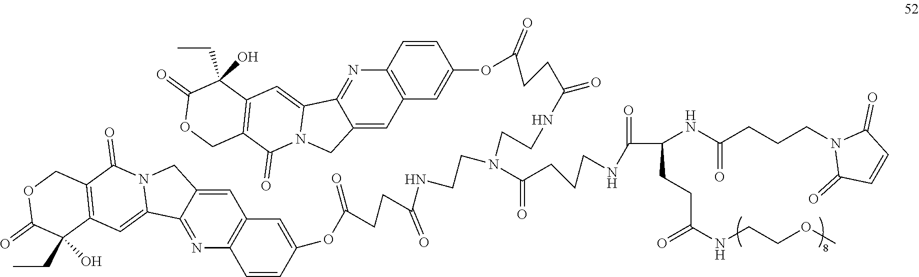

To a solution of compound 51 (614 mg, 0.60 mmol) and compound 19 (470 mg, 0.60 mmol) in DMF (20 mL), triethylamine (249 mg, 2.5 mmol) and HATU (234 mg, 0.60 mmol) were added in sequence. The resulting mixture was stirred for 40 minutes, and then concentrated, purified by silica gel column (MeOH/dichloromethane) to give compound 52 (46 mg, 5% yield). ESI-MS m/z: [M+H]⁺ calcd. for C₈₆H₁₀₅N₁₁O₂₈: 17410.81; found 1742.01.

### Example 85. Synthesis of (S)-4-ethyl-4-hydroxy-9-methoxy-1H-pyrano[3′, 4′:6, 7]indolizino[1, 2-b]quinoline-3, 14(4H, 12H)-dione (53)

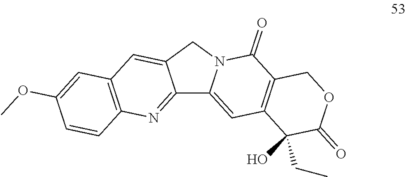 [SMILES: CC[C@@]1(O)C(=O)OCc2c1cc1n(c2=O)Cc2cc3cc(OC)ccc3nc2-1]

10-hydroxycamptothecin (2.5 g, 6.86 mmol) was dissolved in DMF (150 mL), to which potassium carbonate (1.90 g, 13.72 mmol) and methyl iodide (1.17 g, 8.23 mmol) were added, and the reaction was stirred at r.t. overnight. A mixed solvent of petroleum ether (150 mL) and ethyl acetate (150 mL) was added to the reaction mixture and stirred. A yellow solid was precipitated out and collected by filtration, then dispersed in water (20 mL). 1N hydrochloric acid was added dropwise until pH 7, and the mixture was filtered again to give compound 53 (1.0 g, 38% yield). ESI-MS m/z: [M+H]⁺ calcd. for C₂₁H₁₈N₂O₅ 379.38; found 379.05.

### Example 86. Synthesis of bis((S)-4-ethyl-9-methoxy-3, 14-dioxo-3, 4, 12, 14-tetrahydro-1H-pyrano[3′, 4′:6, 7]indolizino[1, 2-b]quinolin-4-yl) (((tert-butoxycarbonyl)azanediyl)bis(ethane-2, 1-diyl))dicarbamate (54)

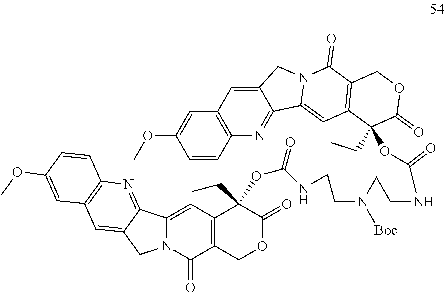 [SMILES: CC[C@@]1(OC(=O)NCCN(CCNC(=O)O[C@]2(CC)C(=O)OCc3c2cc2n(c3=O)Cc3cc4cc(OC)ccc4nc3-2)C(=O)OC(C)(C)C)C(=O)OCc2c1cc1n(c2=O)Cc2cc3cc(OC)ccc3nc2-1]

Compound 53 (350 mg, 0.9 mmol), 4-dimethylaminopyridine (339 mg, 2.8 mmol) and triphosgene (93 mg, 0.34 mmol) were crushed and mixed evenly under N₂, anhydrous dichloromethane (8 mL) was then added dropwise and stirred for 10 minutes. A solution of compound 48 (64 mg, 0.34 mmol) dissolved in anhydrous dichloromethane (4 mL) was added to the mixture, followed by triethylamine (93 mg, 0.9 mmol). After stirring for 15 minutes, the solution was concentrated, and purified by silica gel column (MeOH/dichloromethane) to give compound 54 (200 mg, 22% yield). ESI-MS m/z: [M+H]⁺ calcd. for C₅₃H₅₃N₇O₁₄: 1013.03; found 1013.26.

### Example 87. Synthesis of bis((S)-4-ethyl-9-methoxy-3, 14-dioxo-3, 4, 12, 14-tetrahydro-1H-pyrano[3′, 4′:6, 7]indolizino[1, 2-b]quinolin-4-yl) (azanediylbis(ethane-2, 1-diyl))dicarbamate (55)

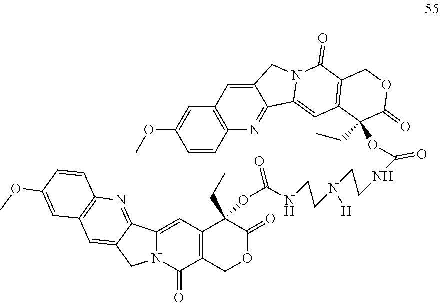 [SMILES: CC[C@@]1(OC(=O)NCCNCCNC(=O)O[C@]2(CC)C(=O)OCc3c2cc2n(c3=O)Cc3cc4cc(OC)ccc4nc3-2)C(=O)OCc2c1cc1n(c2=O)Cc2cc3cc(OC)ccc3nc2-1]

Compound 54 (200 mg, 0.2 mmol) was dissolved in dichloromethane (10 mL), and treated with trifluoroacetic acid (5 mL) for 4 hours. Concentration of the reaction mixture gave compound 55 (0.43 g, 100% yield). ESI-MS m/z: [M+H]⁺ calcd. for C₄₈H₄₅N₇O₁₂: 912.91; found 912.62.

### Example 88. Synthesis of bis((S)-4-ethyl-9-methoxy-3, 14-dioxo-3, 4, 12, 14-tetrahydro-1H-pyrano[3′, 4′:6, 7]indolizino[1, 2-b]quinolin-4-yl) (((4-(2, 5-dioxo-2, 5-dihydro-1H-pyrrol-1-yl)butanoyl)azanediyl)bis(ethane-2, 1-diyl))dicarbamate (56)

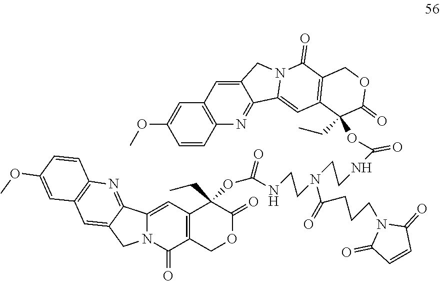 [SMILES: CC[C@@]1(OC(=O)NCCN(CCNC(=O)O[C@]2(CC)C(=O)OCc3c2cc2n(c3=O)Cc3cc4cc(OC)ccc4nc3-2)C(=O)CCCN2C(=O)C=CC2=O)C(=O)OCc2c1cc1n(c2=O)Cc2cc3cc(OC)ccc3nc2-1]

To a solution of compound 55 (249 mg, 0.27 mmol) and 4-(2,5-dioxo-2,5-dihydro-1H-pyrrol-1-yl)butanoic acid (60 mg, 0.32 mmol) in dichloromethane (10 mL), were added triethylamine (112 μL, 0.81 mmol) and HATU (104 mg, 0.27 mmol). The reaction was stirred for 40 minutes, and then washed with water (20 mL). The organic phase was concentrated and purified by preparative HPLC (acetonitrile/water containing formic acid) to give compound 56 (50 mg). ESI-MS m/z: [M+H]+ calcd. for C₅₆H₅₂N₈O₁₅ 1078.06; found 1078.77.

### Example 89. Synthesis of (S)—N, N′-(((((2S, 20S)-11-(tert-butoxycarbonyl)-2, 20-dimethyl-4, 7, 15, 18-tetraoxo-3, 8, 11, 14, 19-pentaazahenicosane-1, 21-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (57)

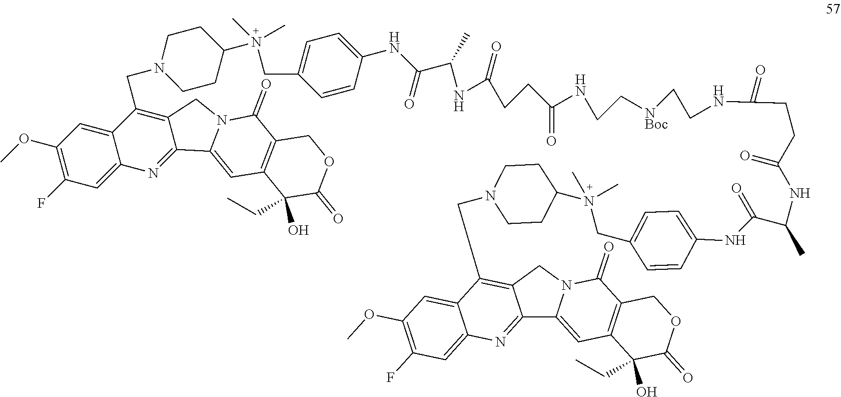

Compound 24 (96 mg, 0.132 mmol) and compound 49 (26 mg, 0.066 mmol were is solved in DMF (3 mL), and cooled to 0° C. HATU (50 mg, 0.132 mmol) and N, N-diisopropylethylamine (46 L, 0.264 mmol) were added, and the reaction was stirred at 0° C. for 30 minutes after addition was completed. The crude reaction mixture was purified directly on preparative HPLC (acetonitrile/water containing formic acid) (acetonitrile/water with 0.1% formic acid) to yield compound 57 (80 mg, 67% yield). ESI-MS m/z: [M]²⁺ calcd. for C₉₁H₁₀₉F₂N₁₅O₁₈: 868.90; found 868.90.

### Example 90. Synthesis of (S)—N, N′-(((((2S, 20S)-2, 20-dimethyl-4, 7, 15, 18-tetraoxo-3, 8, 11, 14, 19-pentaazahenicosane-1, 21-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (58)

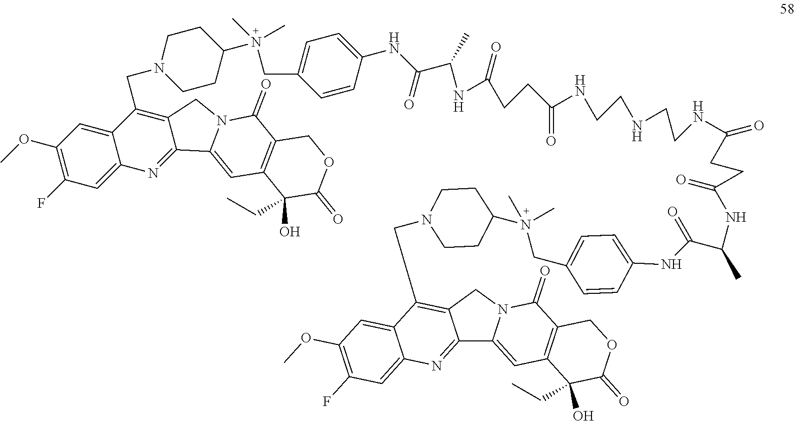

Compound 57 (80 mg, 0.043 mmol) was dissolved in a mixture of dichloromethane and trifluoroacetic acid (3 mL/1 mL), and stirred at r.t. for 30 minutes. Concentration of the reaction mixture afforded compound 58 (100% yield). ESI-MS m/z: [M]²⁺ calcd. for C₈₆H₁₀₁F₂N₁₅O₁₆: 818.87; found818.87.

### Example 91. Synthesis of (S)—N, N′-(((((2S, 20S)-11-((S)-30-(4-(2, 5-dioxo-2, 5-dihydro-1H-pyrrol-1-yl)butanamido)-27, 31-dioxo-2, 5, 8, 11, 14, 17, 20, 23-octaoxa-26, 32-diazahexatriacontan-36-oyl)-2, 20-dimethyl-4, 7, 15, 18-tetraoxo-3, 8, 11, 14, 19-pentaazahenicosane-1, 21-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (59)

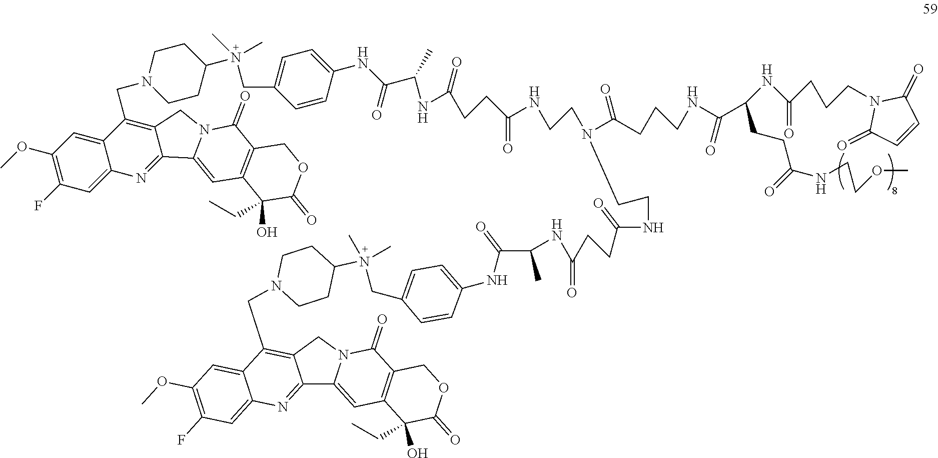

To a solution of compound 58 (74 mg, 0.043 mmol) and compound 19 (39 mg, 0.0516 mmol) in DMF (3 mL), N, N-diisopropylethylamine (15 μL, 0.086 mmol) was added at 0° C. The reaction was warmed to r.t. and stirred for 2 hours. After concentration, the residue was purified by preparative HPLC (acetonitrile/water containing formic acid) to yield compound 59 (12 mg). ESI-MS m/z: [M]²⁺, calcd. for C₁₂₀H₁₅₇F₂N₁₉O₃₀: 1191.06; found 1191.06.

### Example 92. Synthesis of 2, 2′-((tert-butoxycarbonyl)azanediyl)diacetic acid (60)

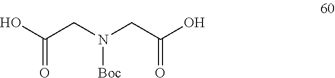 [SMILES: CC(C)(C)OC(=O)N(CC(=O)O)CC(=O)O]

Iminodiacetic acid (5.0 g, 37.6 mmol) was dissolved in THF (50 mL) and water (50 mL), mixed with NaHCO₃ (12.6 g, 150 mmol). Boc₂O (9.8 g, 45.1 mmol) was added slowly at about 5° C., then the reaction was warmed to r.t. and stirred for 2 days. The reaction mixture was diluted with water (100 mL), washed with ethyl acetate (2×30 mL), and then adjusted to pH 1.0 using concentrated HCl. The solution was extracted with ethyl acetate (3×50 mL) and the combined organic phase was washed with water (50 mL), dried over anhydrous Na₂SO₄, filtered and concentrated, triturated with ethyl acetate/petroleum ether to give a white solid (5.5 g, 63% yield). ESI-MS m/z: [M+H]⁺ calcd. for C₉H₁₅NO₆: 234.09; found 234.09.

### Example 93. Synthesis of (S)-1, 1′-(((((2S, 2's)-2, 2′-((2, 2′-((tert-butoxycarbonyl)azanediyl)bis(acetyl))bis(azanediyl))bis(propanoyl))bis(azanediyl))bis(4, 1-phenylene))bis(methylene))bis(4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium) formate (61)

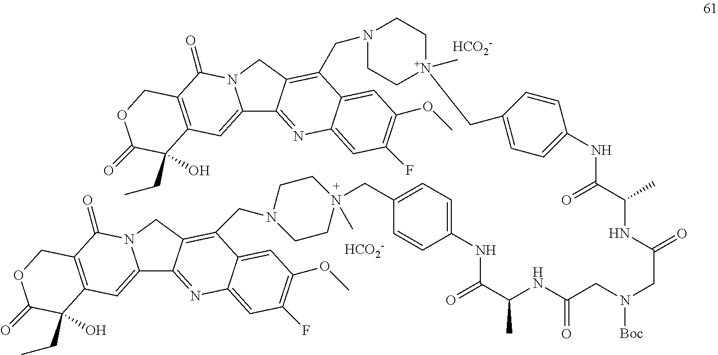

To a solution of compound 36 (109 mg, 0.12 mmol) and compound 60 (14 mg, 0.06 mmol) in DMF (3 mL), cooled to 0° C., were added HATU (50 mg, 0.132 mmol) and N, N-diisopropylethylamine (84 μL, 0.48 mmol). The reaction was stirred at 0° C. for 30 min, and then purified by preparative C-18 HPLC (acetonitrile/water containing formic acid) to give compound 61 (61 mg, 62% yield). ESI-MS m/z: [M]²⁺ calcd. for C₈₃H₉₅F₂N₁₃O₁₆: 783.85; found 783.85.

### Example 94. Synthesis of (S)-1, 1′-(((((2S, 2's)-2, 2′-((2, 2′-azanediylbis(acetyl))bis(azanediyl))bis(propanoyl))bis(azanediyl))bis(4, 1-phenylene))bis(methylene))bis(4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium) formate (62)

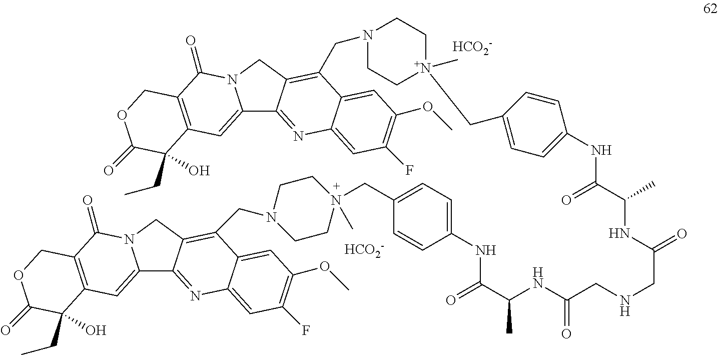

Compound 61 (61 mg, 0.036 mmol) was dissolved in TFA/DCM (1 mL/3 mL) and stirred at r.t. for 30 min. The reaction mixture was diluted with toluene (4 mL) and concentrated to dryness, yielding compound 62 (59.3 mg, >100% yield). ESI-MS m/z: [M]²⁺ calcd. for C₇₈H₈₇F₂N₁₃O₁₄: 733.82; found 733.82.

### Example 95. Synthesis of 1-(4-((30S, 41S)-30-(4-(2, 5-dioxo-2, 5-dihydro-1H-pyrrol-1-yl)butanamido)-37-(2-(((S)-1-((4-((4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methyl-piperazin-1-ium-1-yl)methyl)phenyl)amino)-1-oxopropan-2-yl)amino)-2-oxoethyl)-41-methyl-27, 31, 36, 39-tetraoxo-2, 5, 8, 11, 14, 17, 20, 23-octaoxa-26, 32, 37, 40-tetraazadotetracontanamido)-benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium formate (63)

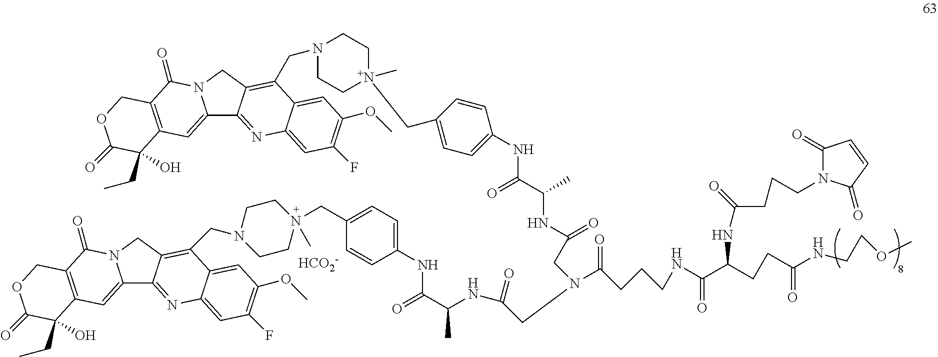

To a solution of compound 62 (65 mg, 0.036 mmol) and compound 18 (27 mg, 0.036 mmol) in DMF (3 mL), cooled to 0° C., were added HATU (17.5 mg, 0.046 mmol) and N, N-diisopropylethylamine (26 μL, 0.144 mmol). The reaction was stirred at 0° C. for 30 min, and then purified by preparative C-18 HPLC (acetonitrile/water containing 2% formic acid) to give compound 63 (39 mg, 62% yield). ESI-MS m/z: [M]²⁺ calcd. for C₁₁₂H₁₄₃F₂N₁₇O₂₈: 1106.01; found 1106.01.

### Example 96. Synthesis of (S)—N, N′-(((((2S, 2's)-2, 2′-((2, 2′-((tert-butoxycarbonyl)azanediyl)bis(acetyl))bis(azanediyl))bis(propanoyl))bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) formate (64)

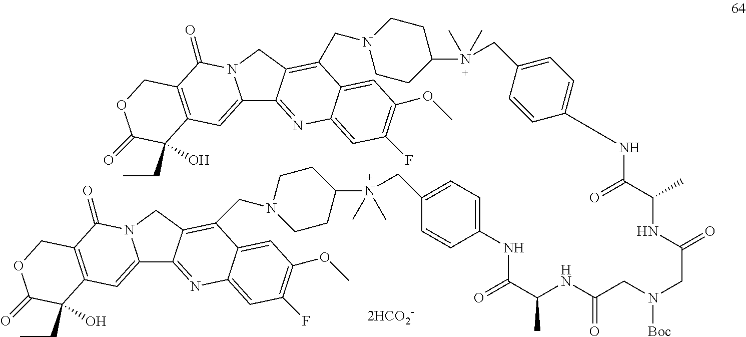

To a solution of compound 24 (106 mg, 0.113 mmol) and compound 60 (13 mg, 0.056 mmol) in DMF (3 mL), cooled to 0° C., were added HATU (43 mg, 0.113 mmol) and N, N-diisopropylethylamine (39 μL, 0.226 mmol). The reaction was stirred for 4 h, and then purified by preparative C-18 HPLC (acetonitrile/water containing formic acid) to give compound 64 (71 mg, 74% yield). ESI-MS m/z: [M]²⁺ calcd. for C₈₇H₁₀₃F₂N₁₃O₁₆: 811.8801; found 811.8875.

### Example 97. Synthesis of (S)—N, N′-(((((2S, 2's)-2, 2′-((2, 2′-azanediylbis(acetyl))bis-(azanediyl))bis(propanoyl))bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino-[1, 2-b]quinolin-11-yl)methyl)-N, N-dimethylpiperidin-4-aminium) formate (65)

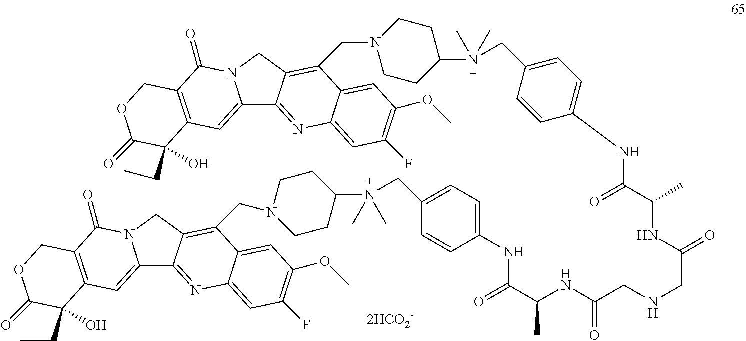

Compound 64 (71 mg, 0.041 mmol) was dissolved in TFA/DCM (1 mL/3 mL) and stirred at r.t. for 30 min. The reaction mixture was diluted with toluene (5 mL) and concentrated to dryness, yielding compound 65 (70 mg, >100 yield). ESI-MS m/z: [M]² calcd. for C₈₂H₉₅F₂N₁₃O₁₄: 761.8539; found 761.8595.

### Example 98. Synthesis of N-(4-((30S, 41S)-30-(4-(2, 5-dioxo-2, 5-dihydro-1H-pyrrol-1-yl)butanamido)-37-(2-(((S)-1-((4-(((1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)dimethylammonio)methyl)phenyl)amino)-1-oxopropan-2-yl)amino)-2-oxoethyl)-41-methyl-27, 31, 36, 39-tetraoxo-2, 5, 8, 11, 14, 17, 20, 23-octaoxa-26, 32, 37, 40-tetraazadotetracontanamido)-benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium formate (66)

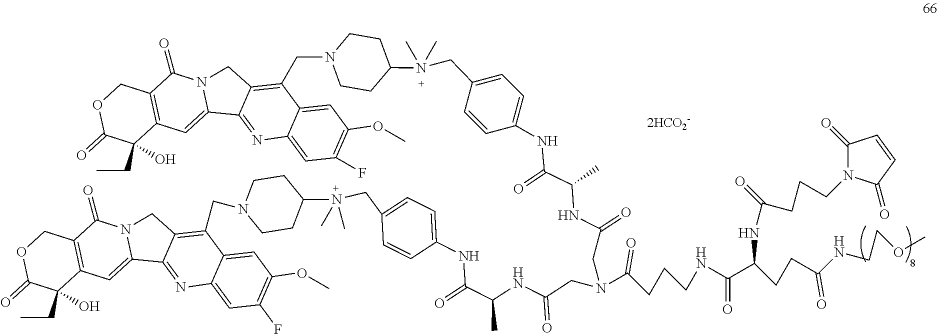

To a solution of compound 65 (70 mg, ˜0.041 mmol) and compound 18 (32 mg, 0.041 mmol) in DMF (4 mL), cooled to 0° C., were added HATU (19 mg, 0.049 mmol) and N, N-diisopropylethylamine (28 μL, 0.164 mmol). The reaction was stirred for 4 h, and then purified by preparative C-18 HPLC (acetonitrile/water containing formic acid) to give compound 66 (43 mg, 45% yield). ESI-MS m/z: [M]²⁺ calcd. for C₁₁₆H₁₅₁F₂N₁₇O₂₈: 1134.04; found 1134.04.

### Example 99. Synthesis of 4-((S)-2-((tert-butoxycarbonyl)amino)propanamido) benzyl (((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)carbamate (67)

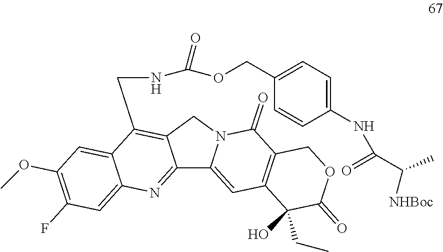 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)NC(=O)OC(C)(C)C)cc1]

To a solution of compound 3 (83 mg, 0.282 mmol) in DCM (2 mL) were added triphosgene (30 mg, 0.094 mmol) and triethylamine (37 μL, 0.282 mmol). The reaction was then warmed to r.t. and stirred for 1 h, concentrated to dryness. Compound 26 (100 mg, 0.235 mmol) was dissolved in DMF (2 mL) and cooled to 0° C., to which triethylamine (37 μL, 0.282 mmol) and the above chloroformate were added. After the addition was completed, the resulting mixture was stirred at 0° C. for 1 h and then purified by preparative C-18 HPLC (acetonitrile/water containing formic acid) to give compound 67 (122 mg, 70% yield). ESI-MS m/z: [M+H]⁺ calcd. for C₃₈H₄₀FN₅O₁₀: 746.2838; found 746.2898.

### Example 100. Synthesis of 4-((S)-2-aminopropanamido)benzyl (((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)carbamate (68)

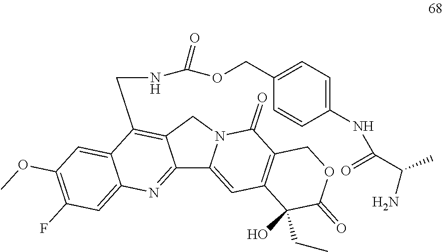 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)N)cc1]

Compound 67 (122.5 mg, 0.164 mmol) was dissolved in TFA/DCM (1 mL/3 mL) and stirred at r.t. for 30 min. The reaction mixture was diluted with toluene (4 mL) and concentrated to dryness, yielding compound 68 (120.2 mg, 0.100% yield). ESI-MS m/z: [M+H]⁺ calcd. for C₃₃H₃₂FN₅O₈: 646.22; found 646.22.

### Example 101. Synthesis of tert-butyl bis(2-(((S)-1-((4-((((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)carbamoyl)oxy)methyl)phenyl)amino)-1-oxopropan-2-yl)amino)-2-oxoethyl)carbamate (69)

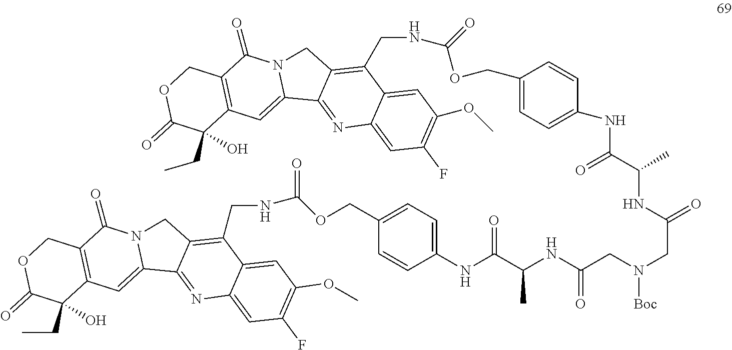 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)NC(=O)CN(CC(=O)N[C@@H](C)C(=O)Nc2ccc(COC(=O)NCc3c4c(nc5cc(F)c(OC)cc35)-c3cc5c(c(=O)n3C4)COC(=O)[C@]5(O)CC)cc2)C(=O)OC(C)(C)C)cc1]

To a solution of compound 68 (120 mg, 0.164 mmol) and compound 60 (19 mg, 0.082 mmol) in DMF (3 mL), cooled to 0° C., were added HATU (62 mg, 0.164 mmol) and N, N-diisopropylethylamine (57 μL, 0.328 mmol). The reaction was stirred for 8 h, concentrated and then purified by preparative C-18 HPLC (acetonitrile/water containing formic acid) to give compound 69 (171 mg, 70% yield). ESI-MS m/z: [M+H]⁺ calcd. for C₇₅H₇₆F₂N₁₁O₂₀: 1488.5237; found 1488.5295.

### Example 102. Synthesis of ((((2S, 2's)-2, 2′-((2, 2′-azanediylbis(acetyl))bis(azanediyl))-bis(propanoyl))bis(azanediyl))bis(4, 1-phenylene))bis(methylene) bis((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)carbamate) (70)

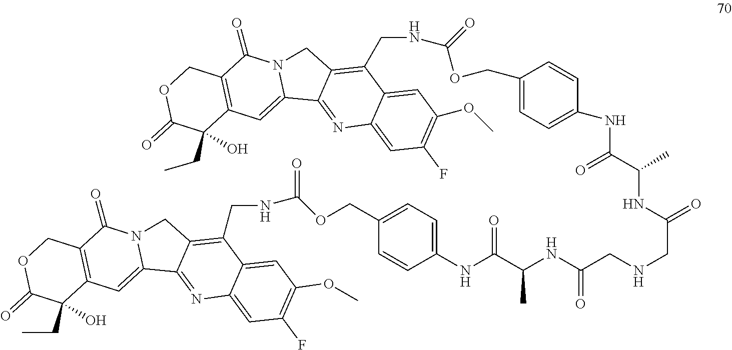 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)NC(=O)CNCC(=O)N[C@@H](C)C(=O)Nc2ccc(COC(=O)NCc3c4c(nc5cc(F)c(OC)cc35)-c3cc5c(c(=O)n3C4)COC(=O)[C@]5(O)CC)cc2)cc1]

Compound 69 (171 mg, 0.115 mmol) was dissolved in TFA/DCM (1 mL/3 mL) and stirred at r.t. for 30 min. The reaction mixture was concentrated to dryness, yielding compound 70 (172 mg, >100% yield). ESI-MS m/z: [M+H]⁺ calcd. for C₇₀H₆₈F₂N₁₁O₁₈: 1388.46; found 1388.46.

### Example 103. Synthesis of ((((2S, 2's)-2, 2′—(((S)-30-(4-(2, 5-dioxo-2, 5-dihydro-1H-pyrrol-1-yl)butanamido)-27, 31, 36-trioxo-37-(2-oxoethyl)-2, 5, 8, 11, 14, 17, 20, 23-octaoxa-26, 32, 37-triazanonatriacontan-39-oyl)bis(azanediyl))bis(propanoyl))bis (azanediyl))bis(4, 1-phenylene))bis(methylene) bis((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)carbamate) (71)

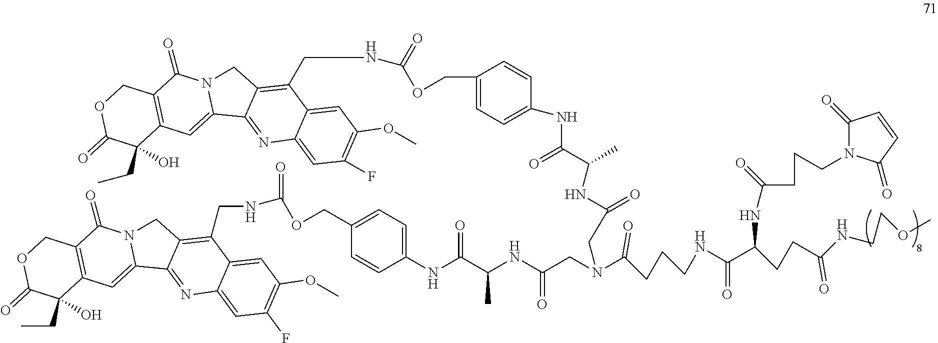

To a solution of compound 70 (172 mg, 0.115 mmol) and compound 18 (87 mg, 0.115 mmol) in DMF (3 mL), cooled to 0° C., were added HATU (52 mg, 0.138 mmol) and N, N-diisopropylethylamine (40 μL, 0.23 mmol). The reaction was stirred for 4 h, and then purified by preparative C-18 HPLC (acetonitrile/water containing formic acid) to give compound 71 (122 mg, 50% yield). ESI-MS m/z: [M+H]⁺ calcd. for C₁₀₄H₁₂₃F₂N₁₅O₃₂: 2132.84; found 2132.84.

### Example 104. Synthesis of (S)-4-ethyl-8-fluoro-4, 9-dihydroxy-11-methyl-1H-pyrano[3′, 4′:6, 7]indolizino[1, 2-b]quinoline-3, 14(4H, 12H)-dione (72)

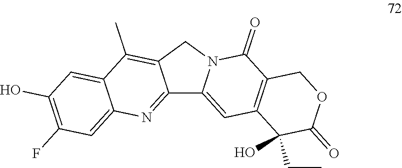 [SMILES: CC[C@@]1(O)C(=O)OCc2c1cc1n(c2=O)Cc2c-1nc1cc(F)c(O)cc1c2C]

1-(2-amino-4-fluoro-5-hydroxyphenyl)ethanone (0.41 g, 2.5 mmol) and (S)-4-ethyl-4-hydroxy-7,8-dihydro-1H-pyrano[3,4-f]indolizine-3,6,10(4H)-trione (0.62 g, 2.5 mmol) were dissolved in anhydrous toluene (40 mL), and p-toluenesulfonic acid (46 mg, 0.25 mmol) was added. The suspension was heated at reflux for 3 days and allowed to cool to r.t. After removal of the solvent, the residue was purified by column chromatography to give compound 72 (0.69 g, 73% yield) as a gray powdery solid. ESI-MS m/z: [M+H]⁺ calcd. for C₂₁H₁₇FN₂O₅: 397.11; found 397.16.

### Example 105. Synthesis of (S)-9-(2-bromoethoxy)-4-ethyl-8-fluoro-4-hydroxy-11-methyl-1H-pyrano[3′, 4′:6, 7]indolizino[1, 2-b]quinoline-3, 14(4H, 12H)-dione (73)

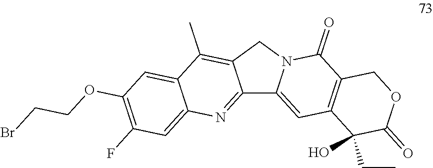 [SMILES: CC[C@@]1(O)C(=O)OCc2c1cc1n(c2=O)Cc2c-1nc1cc(F)c(OCCBr)cc1c2C]

A mixture of compound 72 (0.69 g, 1.74 mmol), anhydrous 1, 2-dibromoethane (6.4 g, 34.8 mmol), and anhydrous K₂CO₃ (1.2 g, 8.7 mmol) in anhydrous DMF (10 mL) was mechanically stirred at 80° C. for 16 h. The reaction mixture was filtered through a pad of Celite, and the filtered residue was washed well with DMF. The combined filtrate and washings were evaporated to dryness in vacuo to afford a dark residue. The residue was purified by column chromatography (0-5% MeOH/dichloromethane) to afford compound 73 (0.74 g, 85%). ESI-MS m/z: [M+H]⁺ calcd. for C₂₃H₂₀BrFN₂O₅: 503.05; found 503.05.

### Example 106. Synthesis of (S)-9-(2-bromoethoxy)-4-ethyl-8-fluoro-4-hydroxy-11-methyl-10-nitro-1H-pyrano[3′, 4′:6, 7]indolizino[1, 2-b]quinoline-3, 14(4H, 12H)-dione (74)

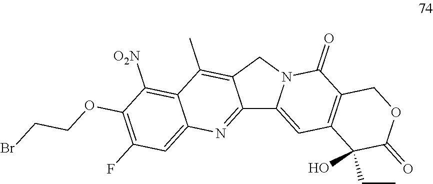 [SMILES: CC[C@@]1(O)C(=O)OCc2c1cc1n(c2=O)Cc2c-1nc1cc(F)c(OCCBr)c([N+](=O)[O-])c1c2C]

To a stirred concentrated H₂SO₄ (1 mL) at 0° C. was added compound 73 (0.74 g, 1.47 mmol) slowly, and the resulting clear solution was cooled to −10° C. A mixture of concentrated H₂SO₄ (0.5 mL) and fuming HNO₃ (0.5 mL), pre-cooled to −10° C., was added dropwise to the cooled reaction mixture at −10° C. The reaction mixture was allowed to warm to 0° C., stirred for an additional 1 h, and then poured slowly onto the ice chips. The yellow precipitate was filtered and washed with H₂O, cold EtOH, and Et₂O. The aqueous filtrate was filtered again through a pad of Celite, and the Celite filter cake was extracted with 30% MeOH/DCM (50 mL). Evaporation of the organic solvent afforded an additional yellow solid. Trituration of the combined yellow solid with EtOH afforded compound 74 (0.74 g, 92%). ESI-MS m/z [M+H]⁺: calcd. for C₂₃H₁₉BrFN₃07: 548.04; found 548.14.

### Example 107. Synthesis of (S)-10-amino-9-(2-bromoethoxy)-4-ethyl-8-fluoro-4-hydroxy-11-methyl-1H-pyrano[3′, 4′:6, 7]indolizino[1, 2-b]quinoline-3, 14(4H, 12H)-dione (75)

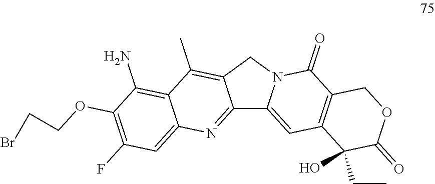 [SMILES: CC[C@@]1(O)C(=O)OCc2c1cc1n(c2=O)Cc2c-1nc1cc(F)c(OCCBr)c(N)c1c2C]

To a stirred concentrated HCl solution (18 mL) at 0° C. was added compound 74 (0.50 g, 0.91 mmol) in small portions, and the resulting clear solution was cooled to −10° C. after 15 min. To the reaction mixture was added SnCl₂ (0.86 g, 4.55 mmol) in small portions and the reaction mixture was allowed to warm to r. t., stirred for 1.5 h, and then poured slowly onto the ice chips. The precipitate was filtered and washed with EtOH and Et₂O, and the aqueous filtrate was extracted with 10% MeOH/DCM. The organic solution was combined with the filtered precipitate dissolved in 30% MeOH/DCM, and then passed through a short silica gel pad and eluted with 30% MeOH/DCM. The organic solvent was removed to afford compound 75 (0.44 g, 94%), which was used in the next step without further purification.

### Example 108. Synthesis of (S)-9-ethyl-5-fluoro-9-hydroxy-16-methyl-2, 3, 12, 15-tetrahydro-[1, 4]oxazino[3, 2-f]pyrano[3′, 4′:6, 7]indolizino[1, 2-b]quinoline-10, 13(1H, 9H)-dione (76)

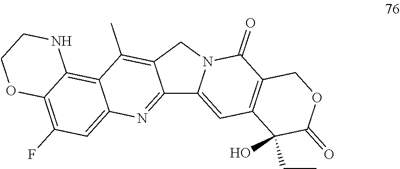 [SMILES: CC[C@@]1(O)C(=O)OCc2c1cc1n(c2=O)Cc2c-1nc1cc(F)c3c(c1c2C)NCCO3]

A solution of compound 75 (0.44 g, 0.85 mmol) in DMSO (4 mL) and NaHCO₃ (0.10 g, 1.19 mmol) was stirred at 70° C. for 4 h, and diluted with HCl (0.1 M, 8 mL) and H₂O (40 mL). The precipitated solid was filtered, dissolved in a small volume of 10% MeOH/DCM, and purified by column chromatography using (1:20-1:6) MeOH/DCM as eluent to afford compound 76 (0.24 g, 66%). ESI-MS m/z: [M+H]⁺ calcd. for C₂₃H₂₀FN₃O₅: 438.14; found 438.14.

### Example 109. Synthesis of (S)-tert-butyl (2-(9-ethyl-5-fluoro-9-hydroxy-16-methyl-10, 13-dioxo-2, 3, 9, 10-tetrahydro-[1, 4]oxazino[3, 2-f]pyrano[3′, 4′:6, 7]indolizino[1, 2-b]quinolin-1(12H, 13H, 15H)-yl)ethyl)carbamate (77)

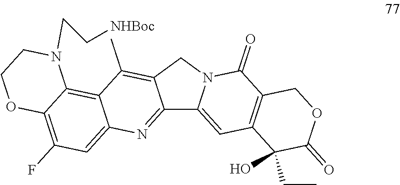 [SMILES: CC[C@@]1(O)C(=O)OCc2c1cc1n(c2=O)Cc2c-1nc1cc(F)c3c(c1c2C)N(CCNC(=O)OC(C)(C)C)CCO3]

To a stirred solution of compound 76 (0.20 g, 0.456 mmol) in anhydrous DMF (2 mL) were added NaI (0.68 g, 4.56 mmol) and tert-butyl (2-chloroethyl)carbamate (0.82 g, 4.56 mmol), and the mixture was heated at 120° C. for 18 h. The reaction mixture was cooled to r. t., evaporated to dryness in vacuo, and purified by column chromatography (0-5% MeOH/DCM) to afford compound 77 (0.19 g, 75%). ESI-MS m/z: [M+H]⁺ calcd. for C₃₀H₃₃FN₄O₇: 581.23; found 581.40.

### Example 110. Synthesis of (S)-1-(2-aminoethyl)-9-ethyl-5-fluoro-9-hydroxy-16-methyl-2, 3, 12, 15-tetrahydro-[1, 4]oxazino[3, 2-f]pyrano[3′, 4′:6, 7]indolizino[1, 2-b]quinoline-10, 13(1H, 9H)-dione (78)

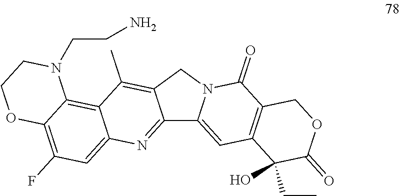 [SMILES: CC[C@@]1(O)C(=O)OCc2c1cc1n(c2=O)Cc2c-1nc1cc(F)c3c(c1c2C)N(CCN)CCO3]

To a solution of compound 77 (0.19 g, 0.327 mmol) in dichloromethane (5 mL) was added TFA (2.5 mL) and the reaction was stirred at r.t. for 30 min. The reaction mixture was concentrated, co-evaporated with dichloromethane for three times to afford compound 78, which was used in the next step without further purification.

### Example 111. Synthesis of Compound 79

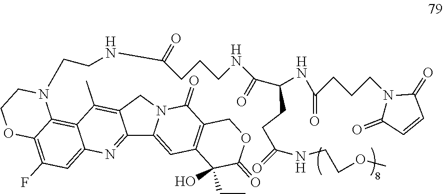

Compound 78 from the previous step and compound 19 (0.45 g, 0.49 mmol) were dissolved in DMF (5 mL), cooled to about 0° C., and then N, N-diisopropylethylamine (172 μL, 0.98 mmol) was added. The reaction was warmed to r.t. and stirred for 2 hours, concentrated, and purified by preparative HPLC (acetonitrile/water containing formic acid) to give compound 79 (359 mg, 60% yield). ESI-MS m/z: [M+H]⁺ calcd. for C₅₉H₈₁FN₈O₁₉: 1224.56; found 1224.78.

### Example 112. Synthesis of 2-amino-4-fluoro-5-hydroxybenzaldehyde (80)

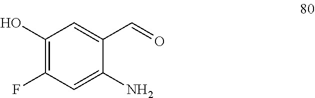 [SMILES: Nc1cc(F)c(O)cc1C=O]

To a solution of 4-fluoro-3-methoxybenzaldehyde (770 mg, 5.0 mmol) in concentrated sulfuric acid (10 mL) at 0° C. was added fuming nitric acid (95%, 315 mg, 4.8 mmol) dropwise. The mixture was stirred at r.t for 1h, then poured into ice water, and filtered. The filter cake was washed with water and then dried. The resulting residue was dissolved in DMF (20 mL), lithium chloride (1.6 g, 25 mmol) was added and the mixture was refluxed for 4 h then poured into water, and concentrated hydrochloric acid was added dropwise to reach pH 4. The solution was extracted with ethyl acetate and the organic layer was washed with brine, dried and concentrated in vacuo. To the resulting residue were added ethanol/water (25 mL, 4:1), iron powder (1.21 g, 22 mmol) and ammonium chloride (433 mg, 8.1 mmol). The mixture was stirred at 80° C. for 2h, and solid was then filtered off. Water was added to the filtrate, and the resulting mixture was extracted with ethyl acetate. The organic layer was washed with brine, dried, and concentrated, purified by column chromatography to give the title compound (125 mg, 16 yield). ESI-MS m/z: [M+H]⁺ calcd. for C₇H₆FNO₂ 156.04; found 156.04.

### Example 113. Synthesis of (S)-4-ethyl-8-fluoro-4, 9-dihydroxy-1H-pyrano[3′, 4′:6, 7]indolizino[l, 2-b]quinoline-3, 14(4H, 12H)-dione (81)

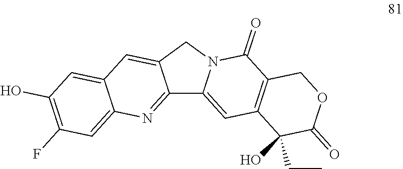 [SMILES: CC[C@@]1(O)C(=O)OCc2c1cc1n(c2=O)Cc2cc3cc(O)c(F)cc3nc2-1]

Compound 80 (0.125 g, 0.805 mmol) and (S)-4-ethyl-4-hydroxy-7,8-dihydro-1H-pyrano[3,4-f]indolizine-3,6,10(4H)-trione (0.202 g, 0.76 mmol) were dissolved in anhydrous toluene (40 mL), and p-toluenesulfonic acid (13 mg, 0.076 mmol) was added. The suspension was heated at reflux for 2 days and allowed to cool to r.t. After removal of about two-thirds of toluene, the residue was filtered and the filter cake was washed with dichloromethane, air-dried to give compound 81 (0.26 g, 90% yield) as a gray powdery solid. ESI-MS m/z: [M+H]⁺ calcd. for C₂₀H₁₆FN₂O₅: 383.10; found 383.10.

### Example 114. Synthesis of (S)-tert-butyl (2-(9-ethyl-5-fluoro-9-hydroxy-10, 13-dioxo-9, 10-dihydro-[1, 3]oxazino[5, 6-f]pyrano[3′, 4′:6, 7]indolizino[1, 2-b]quinolin-2(1H, 3H, 12H, 13H, 15H)-yl)ethyl)carbamate (82)

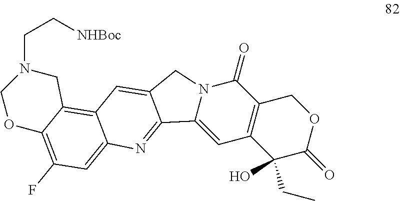 [SMILES: CC[C@@]1(O)C(=O)OCc2c1cc1n(c2=O)Cc2cc3c4c(c(F)cc3nc2-1)OCN(CCNC(=O)OC(C)(C)C)C4]

A solution of N-Boc-ethylenediamine (50 mg, 0.31 mmol) and paraformaldehyde (70 mg, 0.78 mmol) in 1, 4-dioxane (5 mL) was heated at about 100° C. for 2 h, then cooled to r.t. and compound 81 (100 mg, 0.26 mmol) was added. The reaction was heated to 100° C. again and stirred for 2 days, cooled to r.t. and purified by preparative C-18 HPLC (acetonitrile/water containing formic acid) to give compound 82 (117 mg, 80% yield). ESI-MS m/z: [M+H]⁺ calcd. for C₂₉H₃₁FN₄O₇: 567.22; found 567.22.

### Example 115. Synthesis of (S)-2-(2-aminoethyl)-9-ethyl-5-fluoro-9-hydroxy-2, 3, 12, 15-tetrahydro-[1, 3]oxazino[5, 6-f]pyrano[3′, 4′:6, 7]indolizino[1, 2-b]quinoline-10, 13(1H, 9H)-dione (83)

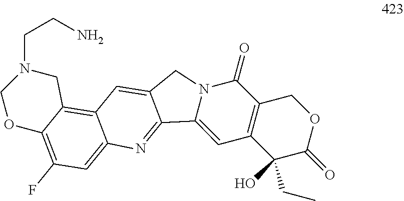 [SMILES: CC[C@@]1(O)C(=O)OCc2c1cc1n(c2=O)Cc2cc3c4c(c(F)cc3nc2-1)OCN(CCN)C4]

Compound 82 (117 mg, 0.208 mmol) was dissolved in TFA/DCM (2 mL/6 mL) and stirred at r.t. for 1 h. The reaction mixture was concentrated to dryness, yielding a yellow solid 83 (117 g, >100 yield). ESI-MS m/z: [M+H]⁺ calcd. for C₂₄H₂₃FN₄05: 467.17; found 467.17.

### Example 116. Synthesis of (S)-2-(4-(2, 5-dioxo-2, 5-dihydro-1H-pyrrol-1-yl)butanamido)-N₁-(4-((2-((S)-9-ethyl-5-fluoro-9-hydroxy-10, 13-dioxo-9, 10-dihydro-[1, 3]oxazino[5, 6-f]pyrano[3′, 4′:6, 7]indolizino[1, 2-b]quinolin-2(1H, 3H, 12H, 13H, 15H)-yl)ethyl)amino)-4-oxobutyl)-N₅-(2, 5, 8, 11, 14, 17, 20, 23-octaoxapentacosan-25-yl)pentanediamide (84)

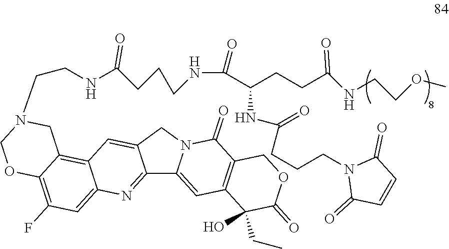

To a solution of compound 83 (120 mg, 0.208 mmol) and compound 19 (193 mg, 0.208 mmol) in DMF (5 mL), cooled to 0° C., was added N, N-diisopropylethylamine (72 μL, 0.416 mmol). The reaction was warmed to r.t. and stirred for 2 h, concentrated and purified by preparative HPLC (acetonitrile/water containing formic acid) to give compound 84 (100 mg, 40% yield). ESI-MS m/z: [M+H]⁺ calcd. for C₅₈H₇₉FN₈O₁₉: 1211.54; found 1211.54.

### Example 117. Synthesis of (S)-9-ethyl-5-fluoro-9-hydroxy-2-(2-hydroxyethyl)-2, 3, 12, 15-tetrahydro-[1, 3]oxazino[5, 6-f]pyrano[3′, 4′:6, 7]indolizino[1, 2-b]quinoline-10, 13(1H, 9H)-dione (85)

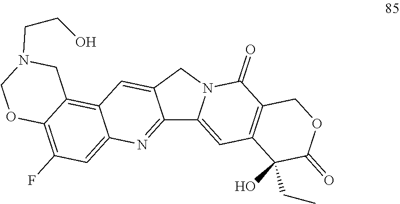

A solution of ethanolamine (19 mg, 0.31 mmol) and paraformaldehyde (70 mg, 0.78 mmol) in 1, 4-dioxane (5 mL) was heated at about 100° C. for 2 h, then cooled to r.t. and compound 81 (100 mg, 0.26 mmol) was added. The reaction was heated to 100° C. again and stirred for 2 days, cooled to r.t. and purified by preparative HPLC (acetonitrile/water containing formic acid) to give compound 85 (91 mg, 75% yield). ESI-MS m/z: [M+H]⁺ calcd. for C₂₄H₂₂FN₃06: 468.15; found 468.15.

### Example 118. Synthesis of (S)—N₁-(4-((2-aminoethyl)amino)-4-oxobutyl)-2-(4-(2, 5-dioxo-2, 5-dihydro-1H-pyrrol-1-yl)butanamido)-N₅-(2, 5, 8, 11, 14, 17, 20, 23-octaoxapentacosan-25-yl)pentanediamide (86)

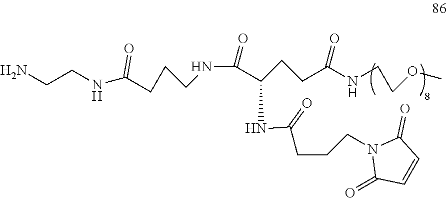

A solution of 1, 2-diethyl-diamine (300 mg, 4.99 mmol) in THF (15 mL) and 1.0 M NaH₂PO₄ (15 mL) was adjusted to pH 7.5 with 0.1 M H₃PO₄. The mixture was cooled to 4˜10° C., and the title compound 19 (700 mg, 0.75 mmol) was added in four portions in 1 h. After additionally stirred for 2 h, the mixture was concentrated and purified by preparative HPLC (acetonitrile/water containing 1% formic acid) to give compound 86 (528 mg, 82% yield). ESI-MS m/z: [M+H]⁺ calcd. for C₃₆H₆₅N₆O₁₄: 805.4560; found 805.4595.

### Example 119. Synthesis of 2-((S)-9-ethyl-5-fluoro-9-hydroxy-10, 13-dioxo-9, 10-dihydro-[1, 3]oxazino[5, 6-f]pyrano[3′, 4′:6, 7]indolizino[1, 2-b]quinolin-2(1H, 3H, 12H, 13H, 15H)-yl)ethyl ((S)-30-(4-(2, 5-dioxo-2, 5-dihydro-1H-pyrrol-1-yl)butanamido)-27, 31, 36-trioxo-2, 5, 8, 11, 14, 17, 20, 23-octaoxa-26, 32, 37-triazanonatriacontan-39-yl)carbamate (87)

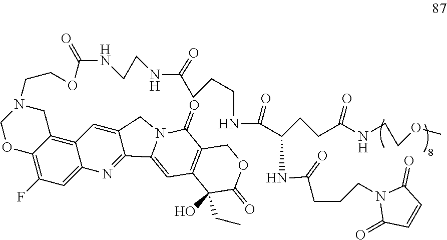

To a solution of compound 85 (30 mg, 0.0642 mmol) in dry THF (5 mL) and DIPEA (15 μl, 0.091 mmol) at 0° C., 4-nitrophenyl carbonochloridate (13 mg, 0.0646 mmol) was added. The mixture was stirred for 4 h at 0° C., and compound 86 (55 mg, 0.0643 mmol) and DIPEA (10 1, 61.2 mmol) were added. The mixture was stirred for 4 h, concentrated and purified by preparative C-18 HPLC (acetonitrile/water containing 1% formic acid) to give compound 87 (39 mg, 47% yield). ESI-MS m/z: [M+H]⁺ calcd. for C₆₁H₈₅FN₉O₂₁: 1298.5845; found 1298.5935.

### Example 120. Synthesis of bis(2, 5-dioxopyrrolidin-1-yl) 4, 4′-((((tert-butoxycarbonyl)azanediyl)bis(ethane-2, 1-diyl))bis(azanediyl))bis(4-oxobutanoate) (88)

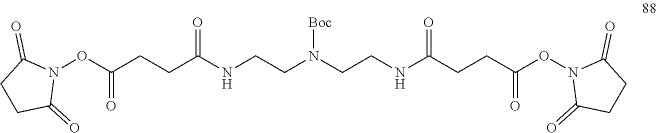 [SMILES: CC(C)(C)OC(=O)N(CCNC(=O)CCC(=O)ON1C(=O)CCC1=O)CCNC(=O)CCC(=O)ON1C(=O)CCC1=O]

To a solution of compound 49 (201 mg, 0.5 mmol) in DCM (10 mL), were added EDC·HCl (287 mg, 1.5 mmol) and NHS (173 mg, 1.5 mmol). The reaction was stirred at r.t. for 1 h and then diluted with DCM (50 mL), washed with water (2×10 mL), dried over anhydrous Na₂SO₄, filtered and concentrated to give compound 88 (297 mg, 100% yield). ESI-MS m/z: [M+H]⁺ calcd. for C₂₅H₃₅N₅O₁₂: 598.22; found 598.22.

### Example 121. Synthesis of 11-(tert-butoxycarbonyl)-4, 7, 15, 18-tetraoxo-3, 8, 11, 14, 19-pentaazahenicosane-1, 21-dioic acid (89)

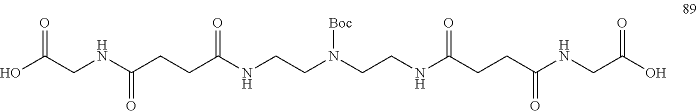 [SMILES: CC(C)(C)OC(=O)N(CCNC(=O)CCC(=O)NCC(=O)O)CCNC(=O)CCC(=O)NCC(=O)O]

H-Gly-OH (94 mg, 1.25 mmol) was dissolved in water (10 mL) and NaHCO₃ (168 mg, 2.00 mmol) was added, followed by compound 88 (297 mg, 0.5 mmol). The reaction was then stirred at r.t. for 1 h and concentrated, purified by preparative HPLC (acetonitrile/water containing formic acid) to give compound 89 (155 mg, 60% yield). ESI-MS m/z: [M+H]⁺ calcd. for C₂₁H₃₅N₅O₁₀: 518.23; found 518.23.

### Example 122. Synthesis of bis(perfluorophenyl) 11-(tert-butoxycarbonyl)-4, 7, 15, 18-tetraoxo-3, 8, 11, 14, 19-pentaazahenicosane-1, 21-dioate (90)

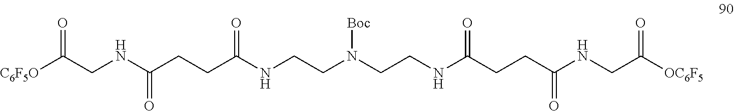

To a solution of compound 89 (110 mg, 0.12 mmol) in DCM (5 mL) were added pentafluorophenol (48 mg, 0.26 mmol) and EDC·HCl (50 mg, 0.26 mmol). The reaction was stirred at r.t. for 2 h and then diluted with DCM (50 mL), washed with water (2×10 mL), dried over anhydrous Na₂SO₄, filtered and concentrated to give compound 90 (180 mg, 100% yield). ESI-MS m/z: [M+H]⁺ calcd. for C₃₃H₃₃F₁₀N₅O₁₀: 850.20; found 850.20.

### Example 123. Synthesis of tert-butyl bis(2-(4-((2-((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)amino)-2-oxoethyl)amino)-4-oxobutanamido)ethyl)carbamate (91)

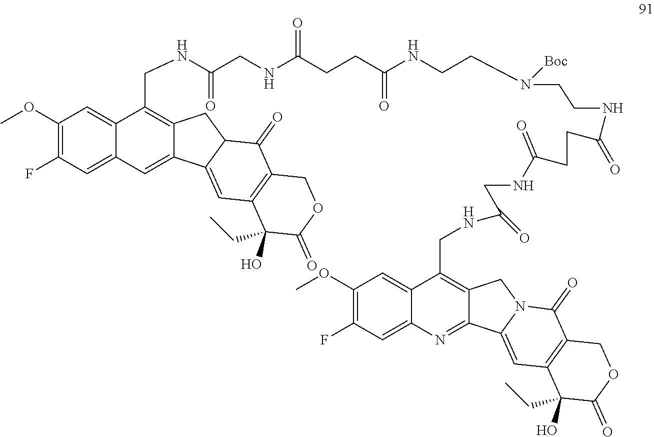 [SMILES: CC[C@@]1(O)C(=O)OCC2=C1C=C1c3cc4cc(F)c(OC)cc4c(CNC(=O)CNC(=O)CCC(=O)NCCN(CCNC(=O)CCC(=O)NCC(=O)NCc4c5c(nc6cc(F)c(OC)cc46)-c4cc6c(c(=O)n4C5)COC(=O)[C@]6(O)CC)C(=O)OC(C)(C)C)c3CC1C2=O]

To a solution of compound 26 (55 mg, 0.13 mmol) in DMF (1 mL) were added DIPEA (27 mg, 0.21 mmol) and compound 90 (50 mg, 0.06 mmol) over an ice-water bath. The reaction was warmed to r.t. and stirred for 1 h, then concentrated, purified by preparative HPLC (acetonitrile/water containing formic acid) to give compound 91 (20 mg, 25% yield). ESI-MS m/z: [M+H]⁺ calcd. for C₆₅H₇₂F₂N₁₁O₁₈: 1332.49; found 1332.49.

### Example 124. Synthesis of N₁, N₁′-(azanediylbis(ethane-2, 1-diyl))bis(N₄-(2-((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)amino)-2-oxoethyl)succinamide) (92)

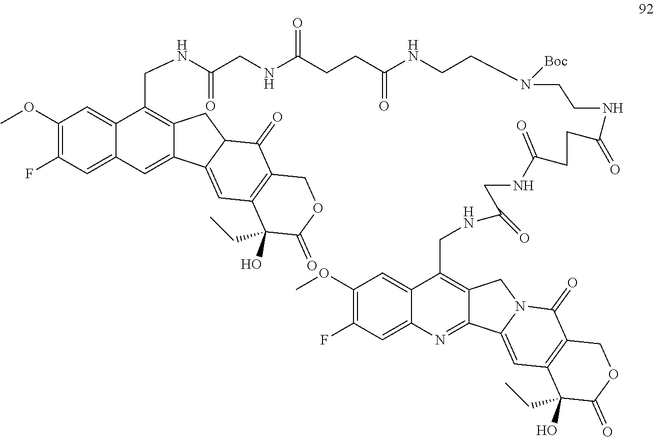 [SMILES: CC[C@@]1(O)C(=O)OCC2=C1C=C1c3cc4cc(F)c(OC)cc4c(CNC(=O)CNC(=O)CCC(=O)NCCN(CCNC(=O)CCC(=O)NCC(=O)NCc4c5c(nc6cc(F)c(OC)cc46)-c4cc6c(c(=O)n4C5)COC(=O)[C@]6(O)CC)C(=O)OC(C)(C)C)c3CC1C2=O]

Compound 91 (20 mg, 0.015 mmol) was dissolved in TFA/DCM (0.5 mL/1 mL) and stirred at r.t. for 2 h. The reaction mixture was concentrated to dryness, yielding a yellow solid (18.5 mg, 100% yield). ESI-MS m/z: [M+H]⁺ calcd. for C₆₀H₆₃F₂N₁₁O₁₆: 1232.44; found 1232.44.

### Example 125. Synthesis of (S)-2-(4-(2, 5-dioxo-2, 5-dihydro-1H-pyrrol-1-yl)butanamido)-N₁-(1-((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)-13-(2-(4-((2-((((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)amino)-2-oxoethyl)amino)-4-oxobutanamido)ethyl)-3, 6, 9, 14-tetraoxo-2, 5, 10, 13-tetraazaheptadecan-17-yl)-N5-(2, 5, 8, 11, 14, 17, 20, 23-octaoxapentacosan-25-yl)pentanediamide (93)

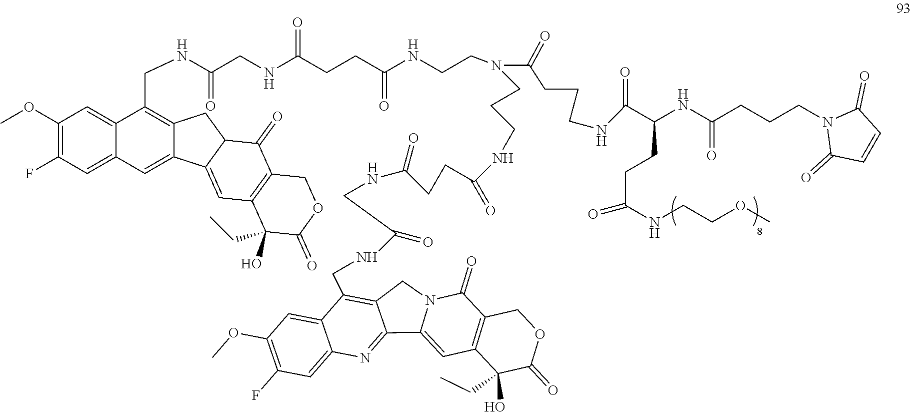

To an ice cold solution of compound 18 (11 mg, 0.015 mmol) in DMF (1 mL), were added HATU (11.4 mg, 0.03 mmol) and N, N-diisopropylethylamine (10 μL, 0.06 mmol), followed by compound 92 (18.5 mg, 0.015 mmol). The reaction was stirred at 0° C. for 1 h, and then purified by preparative HPLC (acetonitrile/water containing formic acid) to give compound 93 (10 mg, 34% yield). ESI-MS m/z: [M+H]⁺ calcd. for C₉₄H₁₁₉F₂N₁₅O₃₀: 1976.82; found 1976.82.

### Example 126. Synthesis of 4-(2-pyridyldithio)-4-methylpentanoic acid (94)

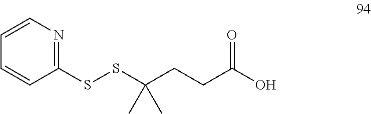 [SMILES: CC(C)(CCC(=O)O)SSc1ccccn1]

4-Mercapto-4-methylpentanoic Acid (Goff, D. et al, BioConjugate Chem. 1990, 1, 381-386) (4.67 g, 31.5 mmol) in MeOH (15 mL) was added the solution of 2, 2′-dithiodipyridine (30.0 g, 136.2 mmol) in the mixture of MeOH (80 mL) and 100 mM sodium phosphate buffer solution (pH 7.5, 70 mL). After stirred for 6 h, the mixture was concentrated, extracted with EtOAc/Hexane (1:1). The aqueous solution was adjusted to pH 3 and extracted with EtOAc (3×100 mL). The organic layers were combined, dried over Na₂SO₄, filtered, evaporated and purified on silica gel column (MeOH/dichloromethane/HOAc, 1:15:0.01) to afford the title compound (7.05 g, 87%). ESI-MS m/z: [M+H]⁺ calcd. for C₁₁H₁₅NO₂S₂ 258.05; found 258.05.

### Example 127. Synthesis of N-Succinimidyl 4-(2-pyridyldithio)-4-methylpentanoate (95)

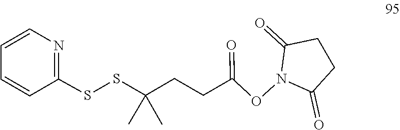 [SMILES: CC(C)(CCC(=O)ON1C(=O)CCC1=O)SSc1ccccn1]

4-(2-pyridyldithio)-4-methylpentanoic acid (2.0 g, 7.78 mmol) in dichloromethane (20 mL) was added N-hydroxysuccimide (1.10 g, 9.56 mmol) and EDC·HCl (4.0 g, 20.8 mmol) and the mixture was stirred overnight, evaporated and purified on silica gel column (EtOAc/dichloromethane, 1:10) to afford the title compound (2.48 g, 90%). ESI-MS m/z: [M+Na]⁺ calcd. for C₁₅H₁₈N₂O₄S2 377.07; found 377.08.

### Example 128. Synthesis of 1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethyl-N-(4-((S)-2-(4-methyl-4-(phenyldisulfanyl)pentanamido) propanamido)benzyl)piperidin-4-aminium (96)

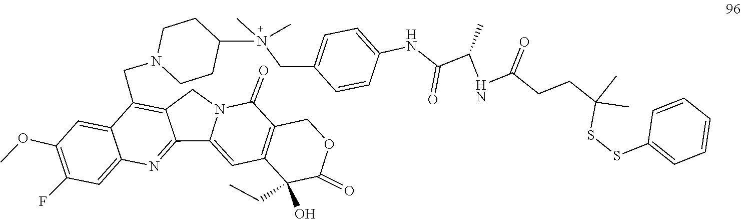

Compound 95 (15 mg, 0.04 mmol) was dissolved in DMA (2 mL), to which compound 24 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 96 (32 mg, 86% yield). ESI-MS m/z: M⁺ calcd. for C₅₁H₆₀FN₆O₇S₂: 951.39; found 951.39.

### Example 129. Synthesis of (S)-4-ethyl-8-fluoro-4, 9-dihydroxy-11-methyl-10-nitro-1H-pyrano[3′, 4′:6, 7]indolizino[1, 2-b]quinoline-3, 14(4H, 12H)-dione (97)

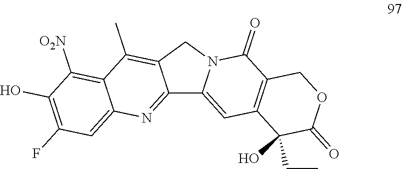 [SMILES: CC[C@@]1(O)C(=O)OCc2c1cc1n(c2=O)Cc2c-1nc1cc(F)c(O)c([N+](=O)[O-])c1c2C]

Compound 72 (451.1 mg, 1.139 mmol) in DCM (10 mL) were added HOAc (1 mL), Ac₂O (0.2 mL) and HNO₃ (conc., 0.3 mL, 4.665 mmol). The mixture was stirred for 3 h, diluted with water (10 mL), separated and the aqueous solution was extracted with DCM (3×25 mL). The organic layers were combined, dried over Na₂SO₄, filtered, and purified on short silica gel column eluted with MeOH/DCM (1:10) to afford the title compound (361.6 mg, 72% yield). ESI-MS m/z: (M+H)⁺ calcd. for C₂₁H₁₇FN₃07: 442.3739; found 442.3810.

### Example 130. Synthesis of (S)-9-(bromomethoxy)-4-ethyl-8-fluoro-4-hydroxy-11-methyl-10-nitro-1H-pyrano[3′, 4′:6, 7]indolizino[1, 2-b]quinoline-3, 14(4H, 12H)-dione (98)

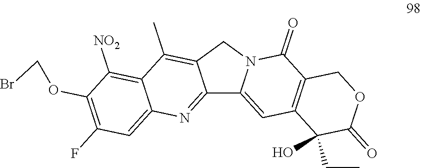 [SMILES: CC[C@@]1(O)C(=O)OCc2c1cc1n(c2=O)Cc2c-1nc1cc(F)c(OCBr)c([N+](=O)[O-])c1c2C]

Compound 97 (350.3 mg, 0.793 mmol), CH₂Br₂ (1 mL, 14.41 mmol) and NaHCO₃ (0.25 g, 2.97 mmol) in THF were stirred at 70° C. for 8 h. The mixture was concentrated and diluted with HCl (0.1 M, 8 mL) and H₂O (40 mL). The precipitated solid was filtered, dissolved in a small volume of (1:10) EtOAc/DCM, and purified by column chromatography using MeOH/DCM (1:10-1:6) as eluent to afford the title compound (0.366 g, 86% yield). ESI-MS m/z: [M+H]⁺ calcd. for C₂₂H₁₈BrFN₃O₇: 534.0313; found 534.0385.

### Example 131. Synthesis of (S)-8-ethyl-4-fluoro-8-hydroxy-15-methyl-11, 14-dihydro-1H-oxazolo[4, 5-f]pyrano[3′, 4′:6, 7]indolizino[1, 2-b]quinoline-9, 12(2H, 8H)-dione (99)

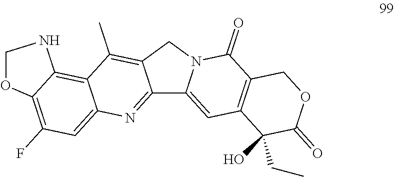

To a stirred mixture of THF (10 mL) and a concentrated HCl solution (5 mL) at 0° C. was added compound 98 (0.360 g, 0.675 mmol) in small portions, and the resulting clear solution was cooled to −10° C. after 15 min. To the reaction mixture was added SnCl₂ (0.384 g, 2.022 mmol) in small portions and the reaction mixture was allowed to warm to r. t., stirred for 1.5 h, and then cooled onto ice. The mixture was neutralized with slowly addition of NaHCO₃ to pH 5.5-6.0 on ice water, followed by refluxing at 70° C. for 6 h and concentrated in vacuo. The precipitate was filtered and washed with EtOH and Et₂O, and the aqueous filtrate was extracted with 10% MeOH/DCM. The organic solution was combined with the filtered precipitate dissolved in 30% MeOH/DCM, and then passed through a short silica gel pad eluted with 20% MeOH/DCM. The organic solvent was removed to afford the title compound (0.120 g, 42% yield in two steps), which was used in the next step without further purification. ESI-MS m/z: [M+H]⁺ calcd. for C₂₂H₁₈FN₃O₅: 424.1309; found 424.1375.

### Example 132. Synthesis of (S)-tert-butyl (2-((2-(8-ethyl-4-fluoro-8-hydroxy-15-methyl-9, 12-dioxo-2, 8, 9, 11, 12, 14-hexahydro-1H-oxazolo[4, 5-f]pyrano[3′, 4′:6, 7]indolizino[1, 2-b]quinolin-1-yl)-2-oxoethyl)amino)-2-oxoethyl)carbamate (100)

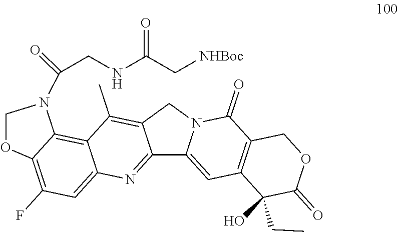

A mixture of compound 99 (158.3 mg, 0.344 mmol), 2-(2-((tert-butoxycarbonyl) amino)acetamido)acetic acid (Boc-Gly-Gly-OH) (103.9 mg, 0.447 mmol) and EDC (153.5 mg, 0.799 mmol) was stirred in DMA (10 mL) for 8 h. The mixture was concentrated and purified on silica gel column eluted with EtOAc/DCM (1:10-1:3) to afford the title compound (182.6 mg, 82% yield). ESI-MS m/z: (M+H) calcd. for C₃₁H₃₃FN₅09: 638.2263; found 638.2295.

### Example 133. Synthesis of (S)-2-amino-N-(2-(8-ethyl-4-fluoro-8-hydroxy-15-methyl-9, 12-dioxo-2, 8, 9, 11, 12, 14-hexahydro-1H-oxazolo[4, 5-f]pyrano[3′, 4′:6, 7]indolizino[1, 2-b]quinolin-1-yl)-2-oxoethyl)acetamide, HCl salt (101)

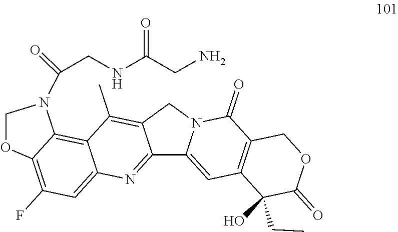

A mixture of compound 100 (175.6 mg, 0.275 mmol), con. HCl solution (1 mL) and dioxane (4 mL) was stirred for 30 min. The mixture was diluted with toluene (5 mL), concentrated and co-evaporated with DCM/toluene (5:5 mL, 2 times) to afford the title compound for the next step without further purification (154.6 mg, 98% yield). ESI-MS m/z: (M+H)⁺ calcd. for C₂₆H₂₅FN₅O₇: 538.1739; found 538.1780.

### Example 134. Synthesis of (R)-2-(4-(2, 5-dioxo-2, 5-dihydro-1H-pyrrol-1-yl)butanamido)-N₁-(4-((2-((2-((S)-8-ethyl-4-fluoro-8-hydroxy-15-methyl-9, 12-dioxo-2, 8, 9, 11, 12, 14-hexahydro-1H-oxazolo[4, 5-f]pyrano[3′, 4′:6, 7]indolizino[1, 2-b]quinolin-1-yl)-2-oxoethyl)amino)-2-oxoethyl)amino)-4-oxobutyl)-N₅-(2, 5, 8, 11, 14, 17, 20, 23-octaoxapentacosan-25-yl)pentanediamide (102)

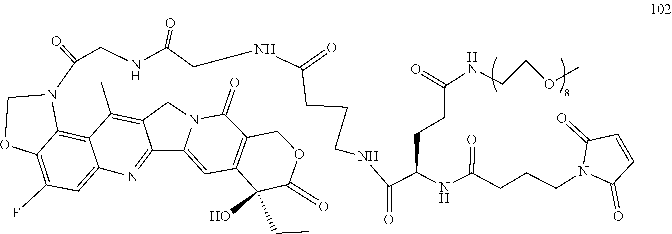

To a solution of compound 101 (47.3 mg, 0.088 mmol) and compound 18 (70.1 mg, 0.092 mmol) in DMF (5 mL), EDC (55 mg, 0.286 mmol) was added. The reaction was stirred for 8 hours. After concentration, the residue was purified by purified on silica gel column eluted with MeOH/DCM (1:6-1:3) to afford the title compound 102 (89.3 mg, 79% yield). ESI-MS m/z: (M+H)⁺ calcd. for C₆₀H₈₁FN₉O₂₁: 1282.5532; found 1282.5590.

### Example 135. Synthesis of 2, 5, 8, 11, 14, 17, 20, 23-octaoxapentacosan-25-yl 4-methylbenzenesulfonate (103)

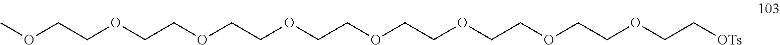 [SMILES: COCCOCCOCCOCCOCCOCCOCCOCCOS(=O)(=O)c1ccc(C)cc1]

To a solution of 2, 5, 8, 11, 14, 17, 20, 23-Octaoxapentacosan-25-ol (50.0 g, 0.130 mol) in dichloromethane (200 mL) and pyridine (100 mL), TsCl (30.2 g, 0.159 mol) was added. The mixture was stirred overnight, evaporated and purified on silica gel column eluted with acetone/dichloromethane (1:1 to 4:1), and dried on a vacuum pump to afford the title compound 57.34 g (82.0% yield). ESI-MS m/z 539.40 ([M+H]⁺).

### Example 136. Synthesis of S-2, 5, 8, 11, 14, 17, 20, 23-octaoxapentacosan-25-yl ethanethioate (104)

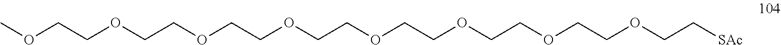

To a solution of 2, 5, 8, 11, 14, 17, 20, 23-octaoxapentacosan-25-yl 4-methylbenzenesulfonate (57.30 g, 0.106 mol) in the mixture of THF (300 mL) and N, N-diisopropylethylamine (50 mL), HSAc (10.0 g, 0.131 mol) was added. The mixture was stirred overnight, evaporated and purified on silica gel column eluted with EtOAc/dichloromethane (1:2 to 4:1), and dried on a vacuum pump to afford the title compound 40.51 g (86% yield). ESI-MS m/z 443.35 ([M+H]⁺).

### Example 137. Synthesis of 2, 5, 8, 11, 14, 17, 20, 23-octaoxapentacosane-25-sulfonic acid (105)

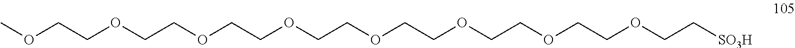 [SMILES: COCCOCCOCCOCCOCCOCCOCCOCCS(=O)(=O)O]

S-2, 5, 8, 11, 14, 17, 20, 23-octaoxapentacosan-25-yl ethanethioate (40.40 g, 0.091 mol) in the mixture of acetic acid (200 mL) and 30% H₂O₂(100 mL) was stirred at 35° C. overnight. The mixture was concentrated, diluted with pure water (200 mL) and toluene (150 mL), separated and the organic layer was extracted with water (2×25 mL). The aqueous solutions were combined, evaporated and dried on a vacuum pump to afford the title compound 40.50 g (99% yield, 95% pure by LC-MS). ESI-MS m/z 449.30 ([M+H]⁺).

### Example 138. Synthesis of 3, 3-N, N-(2″-maleimidoethyl) (2′, 5′, 8′, 11′, 14′, 17′, 20′, 23′, 26′-nonaoxaoctacosane-28′-sulfin)aminopropanoic acid (106)

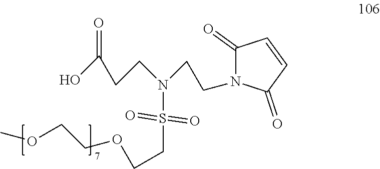

To a solution of 2, 5, 8, 11, 14, 17, 20, 23-octaoxapentacosane-25-sulfonic acid (20.0 g, 44.62 mmol) in the mixture of THF (100 mL) and dichloromethane (100 mL), (COCl)₂ (25.21 g, 200.19 mmol) and DMF (0.015 mL) was added in sequence. The mixture was stirred at r.t. for 2 h, concentrated, co-evaporated with dichloromethane/toluene (1:1, 2×50 mL) and then re-dissolved in THF (50 mL). To the title compound of 3-((2-(2, 5-dioxo-2, 5-dihydro-1H-pyrrol-1-yl)ethyl)amino)-propanoic acid (7.50 g, 35.36 mmol) in THF (100 mL) was added above sulfonyl chloride solution. The mixture was stirred overnight, evaporated in vacuo and purified on silica gel column eluted with MeOH/dichloromethane (1:6 to 1:5), and dried on a vacuum pump to afford the title compound 14.76 g (65% yield). ESI-MS m/z 643.35 ([M+H]⁺).

### Example 139. Synthesis of N—N-succinimido 3, 3-N, N-(2″-maleimidoethyl) (2′, 5′, 8′, 11′, 14′, 17′, 20′, 23′, 26′-nonaoxaoctacosane-28′-sulfin)aminopropanoate (107)

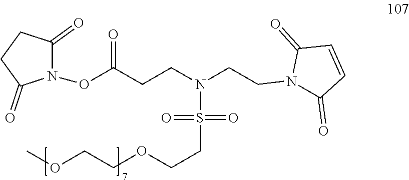

A mixture of compound 106 (7.50 g, 11.67 mmol), N-hydroxysuccinimide (1.50 g, 13.04 mmol) and EDC·HCl (10.10 g, 52.60 mmol) in THF (100 mL) was stirred overnight, evaporated under vacuum and purified on silica gel column eluted with EtOAc/dichloromethane (1:4 to 2:1), and dried on a vacuum pump to afford the title compound 6.30 g (73% yield). ESI-MS m/z 740.40 ([M+H]⁺).

### Example 140. Synthesis of Compound 108

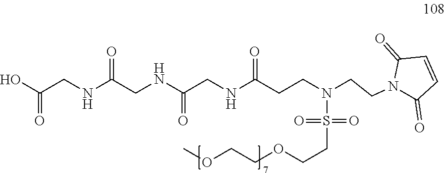

A solution of H-Gly-Gly-Gly-OH (0.50 g, 2.03 mmol) and compound 107 (1.65 g, 2.22 mmol) in DMF (15 mL) at 0° C., N, N-diisopropylethylamine (3 mL) was added. The reaction mixture was stirred at 0° C. for 0.5 h, at r. t. for 4 h. Then the reaction mixture was concentrated, and purified by silica gel chromatography (acetonitrile/water 95:5 with 0.1% formic acid) to afford the title compound (1.04 g, 63% yield). ESI-MS m/z [M+H]⁺: calcd. for C₃₂H₅₆N₅O₁₇S 814.33; found, 814.46.

### Example 141. Synthesis of Compound 109

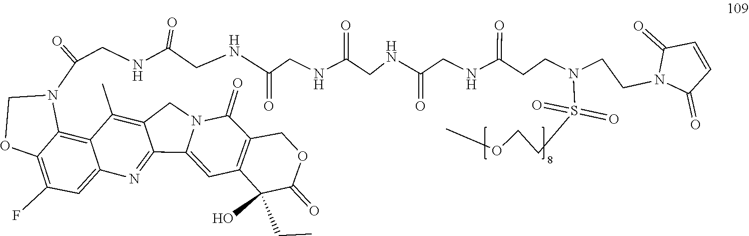

In a solution o compound 108 83.2 mg, 0.102 mmol) and compound 101 (55.1 mg, 0.0960 mmol) in DMA (8 mL) was added EDC (95.5 mg, 0.497 mmol). The mixture was stirred overnight, concentrated and purified on silica gel column eluted with MeOH/DCM (1:6-1:3) to afford the title compound (103.3 mg, 81% yield). ESI-MS m/z: (M+H)⁺ calcd. for C₅₈H₇₈FN₁₀O₂₃S: 1333.4947; found 1333.5015.

### Example 142. Synthesis of Compound 110

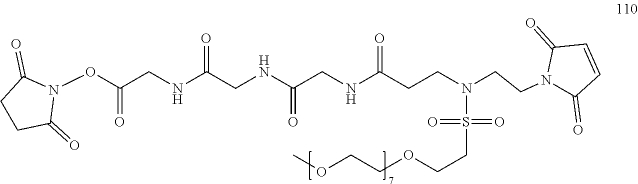

A mixture of compound 108 (0.70 g, 0.86 mmol), N-hydroxysuccinimide (0.20 g, 1.73 mmol) and EDC·HCl (1.21 g, 6.36 mmol) in THF (20 mL) was stirred overnight, evaporated in vacuo and purified on silica gel column, eluted with EtOAc/dichloromethane (1:4 to 2:1), and dried on a vacuum pump to afford the title compound (0.540 g, 69% yield). ESI-MS m/z [M+H]⁺: calcd. for C₃₆H₅₉N₆O₁₉S, 911.34; found 911.42.

### Example 143. Synthesis of Compound 111

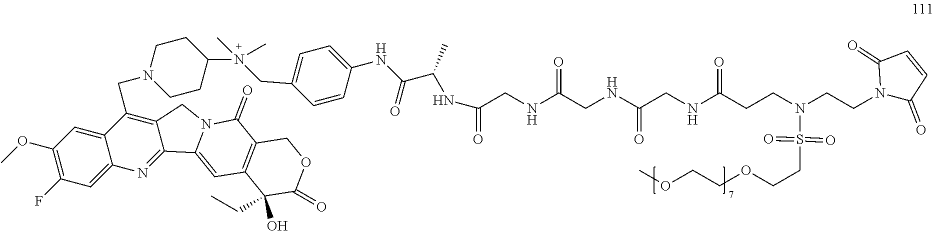

Compound 110 (36 mg, 0.04 mmol) was dissolved in DMF (5 mL), to which compound 24 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 111 (48 mg, 80% yield). ESI-MS m/z: M⁺ calcd. for C₇₁H₉₉FN₁₁O₂₂S 1508.67; found 1508.86.

### Example 144. Synthesis of tert-butyl (2-(2, 5-dioxo-2, 5-dihydro-1H-pyrrol-1-yl)ethyl)carbamate (112)

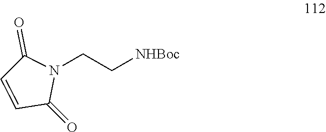 [SMILES: CC(C)(C)OC(=O)NCCN1C(=O)C=CC1=O]

A mixture of N-Boc-ethylenediamine (5.6 mL, 35.4 mmol, 1.1 eq.) and saturated NaHCO₃ (60 mL) was cooled to 0° C., to which N-methoxycarbonyl maleimide (5.00 g, 32.2 mmol, 1.0 eq.) was added in portions. After stirring at 0° C. for 30 min, the reaction was warmed to r.t. and stirred for 1 h. The precipitate was collected by filtration and washed with cold water, then dissolved in ethyl acetate and washed with brine, dried over anhydrous sodium sulfate and concentrated to give a white solid (6.69 g, 87% yield). ESI MS m/z 241.12 ([M+H]).

### Example 145. Synthesis of tert-butyl (2-(1, 3-dioxo-3a, 4, 7, 7a-tetrahydro-1H-4, 7-epoxyisoindol-2(3H)-yl)ethyl)carbamate (113)

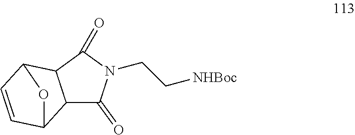 [SMILES: CC(C)(C)OC(=O)NCCN1C(=O)C2C3C=CC(O3)C2C1=O]

In a high pressure tube, a solution of compound 112 (6.00 g, 25.0 mmol), furan (18.0 mL) in toluene (120 mL) was heated to reflux and stirred for 16 h. The colorless solution turned yellow during reaction. The mixture was then cooled to r.t. and concentrated. The resulting white solid was triturated with ethyl ether to give compound 113 (6.5 g, 84% yield). ESI MS m/z 309.13 ([M+H]⁺).

### Example 146. Synthesis of 2-(2-aminoethyl)-3a, 4, 7, 7a-tetrahydro-1H-4, 7-epoxyisoindole-1, 3(2H)-dione hydrochloride (114)

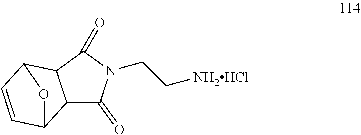

A solution of compound 113 (9.93 g, 32.2 mmol) in dioxane (15 mL) was treated with concentrated HCl (15 mL) at r.t. for 3 h. The reaction was concentrated and the resulting solid was collected by filtration, with washing of the filter cake with ethyl acetate. The solid was dried in an oven (50° C.) overnight to give compound 114 (6.94 g, 88% yield). ESI MS m/z 206.05 ([M+H]⁺).

### Example 147. Synthesis of Compound 115

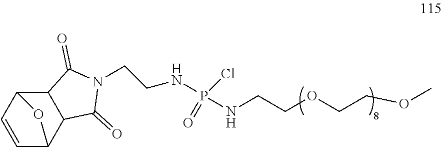

To a solution of compound 114 (1.22 g, 5 mmol) in THF (10 mL) and CH₃CN (10 mL) at −10° C., POCl₃ (0.47 mL, 5 mmol) was added. After stirring for 10 min., 2, 5, 8, 11, 14, 17, 20, 23, 26-nonaoxaoctacosan-28-amine (2.14 g, 5 mmol) was added, followed by DIPEA (0.87 mL, 5 mmol). The reaction was warmed to 0° C. and stirred for 3 h, and then concentrated. The residue was diluted with dichloromethane (10 mL) and filtered over Celite, the filtrate was concentrated in vacuo to afford crude compound (˜3.7 g, ˜50% pure) which was used in the next step directly. ESI MS m/z 716.29 ([M+H]⁺).

### Example 148. Synthesis of Compound 116

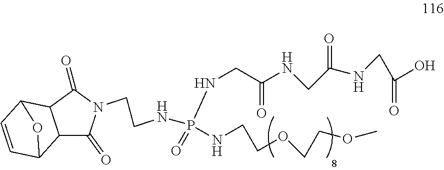

To a solution of 2-(2-(2-aminoacetamido)acetamido)acetic acid (Gly-Gly-Gly, 0.501 g, 2.644 mmol) in CH₃CN (20 mL) and DIPEA (0.87 mL, 5 mmol), compound 115 (1.00 g, 50% pure, ˜0.699 mmol) was added. The mixture was stirred at 40° C. for 6 h, concentrated and purified by preparative HPLC (acetonitrile/water containing formic acid, Φ=5 cm, v=30 mL/min, 70% water to 25% water in 45 min) to give compound 116 (321.5 mg, ˜53% yield). ESI-MS m/z: (M+H)⁺ calcd. for C₃₅H₆₂N₆O₁₇P: 869.3910; found 869.3995.

### Example 149. Synthesis of Compound 117

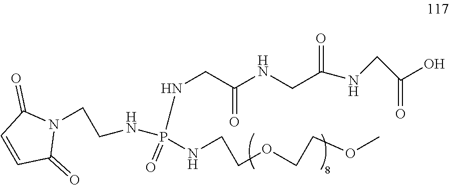

A solution of compound 116 (160.1 mg, 0.184 mmol) in DMA (10 mL) and toluene (10 mL) was refluxed for 8 h, concentrated and purified by preparative C-18 HPLC (acetonitrile/water containing 1% formic acid, Φ=3 cm, v=20 mL/min, 70% water to 25% water in 45 min) to give compound 117 (125.5 mg, 85% yield) after lyophilization. ESI-MS m/z: (M+H)⁺ calcd. for C₃₅H₆₂N₆O₁₇P: 801.3648; found 801.3725.

### Example 150. Synthesis of Compound 118

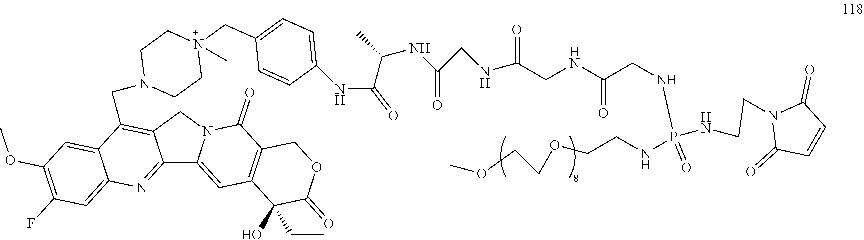

To a solution of compound 36 (50 mg, 0.064 mmol) and compound 117 (51.5 mg, 0.064 mmol) in DMF (5 mL), EDC (99.5 mg, 0.517 mmol) and N, N-diisopropylethylamine (45 μL, 0.26 mmol) were added. The reaction was stirred at r.t for 6 h, concentrated, and purified by preparative C-18 HPLC (acetonitrile/water containing 0.5% formic acid, Φ=3 cm, v=20 mL/min, 70% water to 25% water in 45 min) to give compound 118 (66.7 mg, 71% yield). ESI-MS m/z: M⁺ calcd. for C₄₅H₄₉FN₇O₉: 1467.6607; found 1467.6675.

### Example 151. Synthesis of 5-amino-4-(2-chloroacetyl)-2-methoxy-N-methylbenzamide (119)

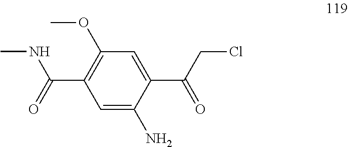 [SMILES: CNC(=O)c1cc(N)c(C(=O)CCl)cc1OC]

A solution of 5-amino-2-methoxy-N-methylbenzamide (5.00 g, 27.76 mmol) dissolved in dichloromethane (20 mL) was added dropwise to an ice water cooled boron trichloride (1 M in dichloromethane, 38.9 mL) solution. The reaction was stirred for 10 minutes and then chloroacetonitrile (3.2 g, 42.5 mmol) and aluminum trichloride (5.2 g, 38.9 mmol) were added. After the addition was completed, the reaction was warmed to r.t. and then refluxed overnight. The reaction mixture was then cooled to about 0° C., quenched with 2 M HCl (80 mL) and stirred at r.t. for 2 hours. Layers were separated and the aqueous phase was extracted with dichloromethane (3×80 mL). Combined organic phases were washed with water (100 mL), dried over sodium sulfate, filtered, concentrated, purified on a C-18 column, eluted with EtOH/H₂O (1:6 to 1:1) to give compound 119 (3.05 g, 43% yield) as a yellow solid. ESI-MS m/z: [M+H]⁺ calcd. for C₁₁H₁₄C₁N₂03: 257.0693; found 257.0725.

### Example 152. Synthesis of (S)-11-(chloromethyl)-4-ethyl-4-hydroxy-9-methoxy-N-methyl-3, 14-dioxo-3, 4, 12, 14-tetrahydro-1H-pyrano[3′, 4′:6, 7]indolizino[1, 2-b]quinoline-8-carboxamide (120)

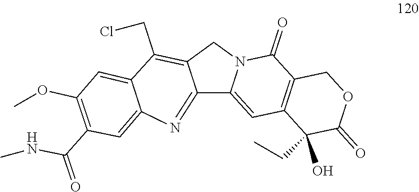 [SMILES: CC[C@@]1(O)C(=O)OCc2c1cc1n(c2=O)Cc2c-1nc1cc(C(=O)NC)c(OC)cc1c2CCl]

Compound 119 (0.59 g, 2.30 mmol) and (S)-4-ethyl-4-hydroxy-7,8-dihydro-1H-pyrano[3,4-f]indolizine-3,6,10(4H)-trione (0.57 g, 2.19 mmol) were dissolved in anhydrous toluene (40 mL), and p-toluenesulfonic acid (42 mg, 0.219 mmol) was added. The suspension was heated at reflux for 2 days and allowed to cool to r.t. After removal of about two-thirds of toluene, the residue was filtered and the filter cake was washed with dichloromethane, air-dried to give compound 120 (0.74 g, 70% yield) as a gray powdery solid. ESI-MS m/z: [M+H]⁺ calcd. for C₂₄H₂₃C₁N₃06: 484.1276; found 484.1220.

### Example 153. Synthesis of N-(4-((S)-2-((tert-butoxycarbonyl)amino)propanamido) benzyl)-1-(((S)-4-ethyl-4-hydroxy-9-methoxy-8-(methylcarbamoyl)-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium, formic acid salt (121)

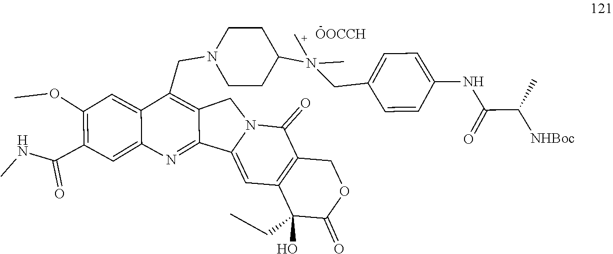

A mixture of compound 120 (238 mg, 0.49 mmol), compound 6 (200 mg, 0.49 mmol) in DMF (5 mL) was stirred at 0° C. for 30 minutes, then triethylamine (63 μL, 0.45 mmol) was added and the stirring was continued for 1 hour. The reaction was concentrated and purification by preparative HPLC (acetonitrile/water containing formic acid, Φ=5 cm, v=30 mL/min, 100% water to 50% water in 45 min) gave compound 121 (242 mg, 55% yield) as a yellow solid. ESI-MS m/z: M⁺ calcd. for C₄₆H₅₈N₇O₉: 852.4291; found 852.4355.

### Example 154. Synthesis of N-(4-((S)-2-aminopropanamido)benzyl)-1-(((S)-4-ethyl-4-hydroxy-9-methoxy-8-(methylcarbamoyl)-3, 14-dioxo-3, 4, 12, 14-tetrahydro-1H-pyrano[3′, 4′:6, 7]-indolizino[1, 2-b]quinolin-11-yl)methyl-N N-dimethylpiperidin-4-aminium trifluoroacetic acid salt (122)

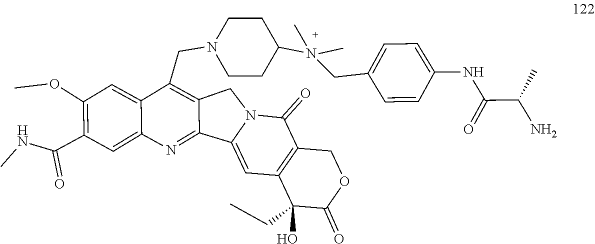

Compound 121 (95 mg, 0.111 mmol) was dissolved in a mixture of dichloromethane and trifluoroacetic acid (2 mL/6 mL), and stirred at r.t. for 30 minutes. The mixture was diluted with toluene (10 mL), then concentrated and dried on a vacuum pump to give compound 122 (108 mg, 100% yield) as a yellow solid. ESI-MS m/z: M⁺ calcd. for C₄₁H₅₀N₇O₇: 752.3766; found 752.3710.

### Example 155. Synthesis of N-(4-((30S, 38S)-30-(4-(2, 5-dioxo-2, 5-dihydro-1H-pyrrol-1-yl)butanamido)-38-methyl-27, 31, 36-trioxo-2, 5, 8, 11, 14, 17, 20, 23-octaoxa-26, 32, 37-triazanonatriacontanamido)benzyl)-1-(((S)-4-ethyl-4-hydroxy-9-methoxy-8-(methylcarbamoyl)-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium formate (123)

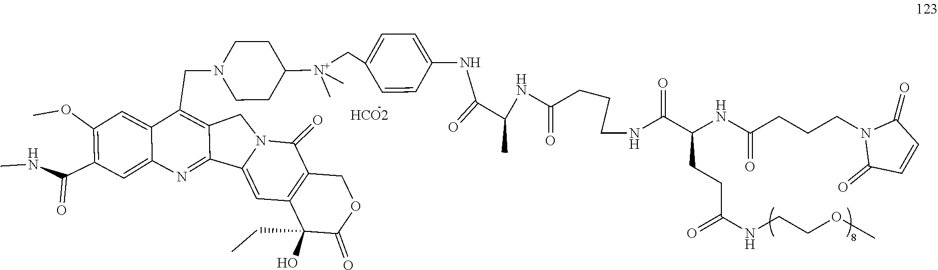

Compound 122 (60 mg, 0.061 mmol) and compound 19 (60 mg, 0.064 mmol) were dissolved in DMF (5 mL), cooled to about 0° C., and then N, N-diisopropylethylamine (21 μL, 0.12 mmol) was added. The reaction was warmed to r.t. and stirred for 2 hours, concentrated, and purified by preparative HPLC (acetonitrile/water containing formic acid, Φ=3 cm, v=20 mL/min, 100% water to 50% water in 45 min) to give compound 123 (38.5 mg, 41% yield). ESI-MS m/z: M⁺ calcd. for C₇₅H₁₀₆N₁₁O₂₁: 1496.7559; found 1496.7595.

### Example 156. Synthesis of meso-2, 3-bis(benzylamino)succinic acid (124)

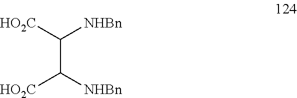

To a solution of meso-2, 3-dibromosuccinic acid (50 g, 181 mmol) in EtOH (400 mL) was added benzylamine (150 mL) dropwise. After completion of addition, the mixture was heated to 90° C. and stirred overnight. The mixture was cooled to r.t. and diluted with water. 6 N HCl was added until pH 4 was reached, to give white precipitates. The precipitates were filtered, rinsed with water and dried to give meso-2, 3-bis(benzylamino)succinic acid (50 g, 152 mmol, 84%).

### Example 157. Synthesis of meso-2, 3-diaminosuccinic acid (125)

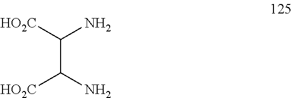

To a solution of meso-2, 3-bis(benzylamino)succinic acid (18 g, 55 mmol) in AcOH (100 mL) and HCl (100 mL) was added Pd/C (3 g, 10 wt %), and the mixture was stirred under 1 atm hydrogen atmosphere at 50° C. for 48 h. The catalyst was removed by filtration and washed with water. The filtrate was concentrated and the residue was dissolved in 1N NaOH (200 mL). Acetic acid was added until pH 5 was reached, to give white precipitates. The precipitates were filtered, rinsed with water and dried to give meso-2, 3-diaminosuccinic acid (8.7 g, >100%).

### Example 158. Synthesis of meso-2, 3-bis(((benzyloxy)carbonyl)amino)succinic acid (126)

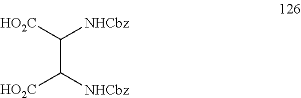 [SMILES: O=C(NC(C(=O)O)C(NC(=O)OCc1ccccc1)C(=O)O)OCc1ccccc1]

To a solution of meso-2, 3-diaminosuccinic acid (31.74 g, 214 mmol) in THF (220 mL) and 4 N NaOH (214 mL) was added benzyl chloroformate (61 mL, 428 mmol) dropwise at 0° C. After completion of the addition, the mixture was allowed to warm to r.t. and stirred for 2 h. The reaction was diluted with water (1600 mL) and washed with ethyl acetate (2×1500 mL). The aqueous layer was separated and acidified with con·HCl until pH 2 was reached. The resultant solution was stirred for 1 h and stood at 5° C. to give white precipitates. The precipitates were filtered, rinsed with water and dried to give meso-2, 3-bis(((benzyloxy)carbonyl)amino)succinic acid (52.2 g, 125 mmol, 59%).

### Example 159. Synthesis of dibenzyl ((3R, 4S)-2, 5-dioxotetrahydrofuran-3, 4-diyl)-dicarbamate

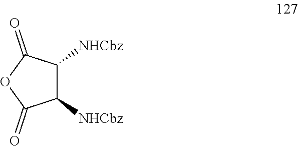 [SMILES: O=C(N[C@H]1C(=O)OC(=O)[C@@H]1NC(=O)OCc1ccccc1)OCc1ccccc1]

The solution of meso-2, 3-bis(benzyloxyl)carbonyl)amino)succinic acid (5 0 g, 12 mmol) in Ac₂O (37.5 mL) was refluxed for 20 min, cooled and concentrated to give an anhydride. The diastereomeric mixture was treat with CHCl₃ (37 mL), the insoluble meso-isomer was filtered and the filtrate was treated with petroleum ether to give crystals of dibenzyl ((3R, 4S)-2, 5-dioxotetrahydrofuran-3, 4-diyl)dicarbamate (racemic mixture, 2.0 g, 5 mmol, 42%).

### Example 160. Synthesis of di-tert-butyl 4, 4′-(((2S, 3S)-2, 3-bis(((benzyloxy) carbonyl)amino)succinyl)bis(azanediyl))dibutanoate (128)

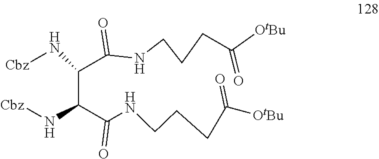 [SMILES: CC(C)(C)OC(=O)CCCNC(=O)[C@@H](NC(=O)OCc1ccccc1)[C@H](NC(=O)OCc1ccccc1)C(=O)NCCCC(=O)OC(C)(C)C]

To solution of dibenzyl ((3S, 4S)-2, 5-dioxotetrahydrofuran-3, 4-diyl)dicarbamate (200 mg, 0.5 mmol) in DMF (5 mL) at about 0° C., tert-butyl aminobutyrate (80 mg, 0.5 mmol) was added. The mixture was stirred at 0° C. for 30 min and then room temperature for 30 min. The reaction solution was re-cooled to about 0° C., followed by addition of DIPEA (64 mg, 0.5 mmol), tert-butyl aminobutyrate (80 mg, 0.5 mmol) and HATU (190 mg, 0.5 mmol). The reaction mixture was warmed to room temperature and stirred for 2 hours, then diluted with dichloromethane (50 mL), washed with saturated NaHCO₃ (20 mL), water (10 mL), dried over anhydrous sodium sulfate, filtered and concentrated. The residue was purified by column chromatography (dichloromethane/MeOH=100:0 to 10:1) to give the title compound (262 mg, 75% yield). MS-ESI m/z: [M+H]⁺ calcd. for C₃₆H₅₀N₄O₁₀, 699.35; found, 699.35.

### Example 161. Synthesis of di-tert-butyl 4, 4′-(((2S, 3S)-2, 3-diaminosuccinyl) bis(azanediyl))-dibutanoate (129)

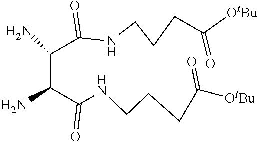 [SMILES: CC(C)(C)OC(=O)CCCNC(=O)[C@@H](N)[C@H](N)C(=O)NCCCC(=O)OC(C)(C)C]

A mixture of compound above (100 mg, 0.14 mmol) and 10% palladium carbon (10 mg) in methanol (5 mL) were stirred under hydrogen (5 psi) overnight. The solid was filtered off and filtrated solution was concentrated to give a colorless oil title compound for the next step without purification (61 mg, 100% yield). MS-ESI m/z: [M+H]⁺ calcd. for C₂₀H₃₈N₄O₆, 431.28; found, 431.28.

### Example 162. Synthesis of di-tert-butyl 4, 4′-(((2S, 3S)-2, 3-bis(4-(2, 5-dioxo-2, 5-dihydro-1H-pyrrol-1-yl)butanamido)succinyl)bis(azanediyl))dibutanoate (130)

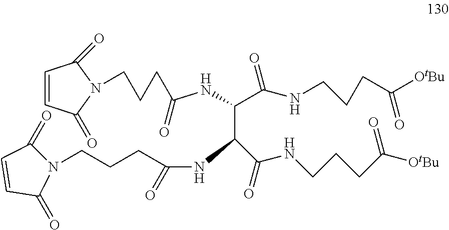 [SMILES: CC(C)(C)OC(=O)CCCNC(=O)[C@@H](NC(=O)CCCN1C(=O)C=CC1=O)[C@H](NC(=O)CCCN1C(=O)C=CC1=O)C(=O)NCCCC(=O)OC(C)(C)C]

To solution of compound 129 (61 mg, 0.14 mmol) in the mixture of ethanol (5 mL) and PBS (0.1 M, pH 7.5, 1.0 mL), 2, 5-dioxopyrrolidin-1-yl 4-(2, 5-dioxo-2, 5-dihydro-1H-pyrrol-1-yl)butanoate (118 mg, 0.42 mmol) was added. The reaction mixture was stirred overnight, concentrated and purified on a silica gel column (dichloromethane/MeOH=100:0 to 10:1) to afford the title compound (65 mg, 60% yield). MS-ESI m/z: [M+H]⁺ calcd. for C₃₇H₅₆N₆O₁₂, 777.40; found, 777.41.

### Example 163. Synthesis of 4, 4′-(((2S, 3S)-2, 3-bis(4-(2, 5-dioxo-2, 5-dihydro-1H-pyrrol-1-yl) butanamido)succinyl)bis(azanediyl))dibutanoic acid (131)

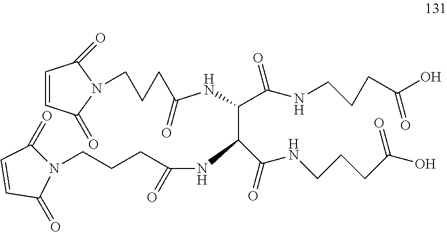 [SMILES: O=C(O)CCCNC(=O)[C@@H](NC(=O)CCCN1C(=O)C=CC1=O)[C@H](NC(=O)CCCN1C(=O)C=CC1=O)C(=O)NCCCC(=O)O]

Compound 130 (65 mg, 0.083 mmol) was dissolved in dichloromethane (6 mL), and treated with trifluoroacetic acid (2 mL) for 2 hours. The reaction mixture was diluted with toluene (5 mL), concentrated to give the title compound (53 mg, 100% yield). MS-ESI m/z: [M+H]⁺ calcd. for C₂₈H₃₆N₆O₁₂, 649.24; found, 649.24.

### Example 164. Synthesis of bis(2, 5-dioxopyrrolidin-1-yl) 4, 4′-(((2R, 3R)-2, 3-bis(4-(2, 5-dioxo-2, 5-dihydro-1H-pyrrol-1-yl)butanamido)succinyl)bis(azanediyl))dibutanoate (132)

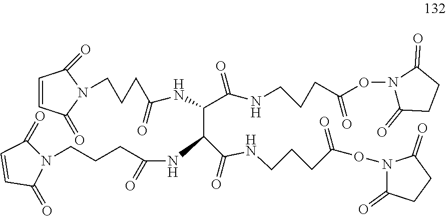 [SMILES: O=C(CCCN1C(=O)C=CC1=O)N[C@H](C(=O)NCCCC(=O)ON1C(=O)CCC1=O)[C@H](NC(=O)CCCN1C(=O)C=CC1=O)C(=O)NCCCC(=O)ON1C(=O)CCC1=O]

To a solution of compound 131 (1.10 g, 1.69 mmol) in DMA (30 mL) were added N-hydroxysuccinimide (1-hydroxypyrrolidine-2, 5-dione) (0.58 g, 5.08 mmol) and EDC·HCl (1.25 g, 6.54 mmol). The mixture was stirred overnight, concentrated and purified on silica gel column, eluted with EtOAc/DCM (1:10) to afford the title compound (1.30 g, 91% yield). ESI-MS m/z [M+H]⁺: calcd. for C₃₆H₄₂NO₁₆ 843.27, found 843.50.

### Example 165. Synthesis of (S)—N, N′-(((((2S, 10S, 11S, 19S)-10, 11-bis(4-(2, 5-dioxo-2, 5-dihydro-1H-pyrrol-1-yl)butanamido)-2, 19-dimethyl-4, 9, 12, 17-tetraoxo-3, 8, 13, 18-tetraazaicosane-1, 20-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (133)

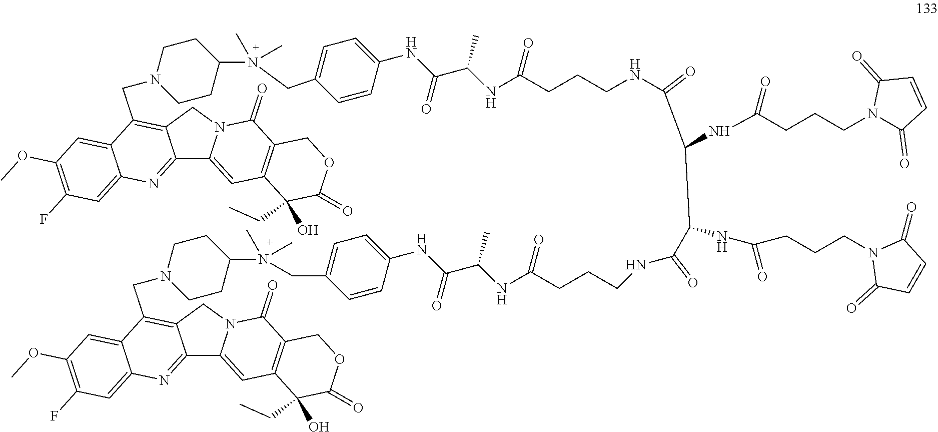

Compound 24 (94 mg, 0.12 mmol) and compound 132 (55 mg, 0.066 mmol) were dissolved in DMA (5 mL), cooled to about 0° C., and then N, N-diisopropylethylamine (84 μL, 0.48 mmol) was added. The reaction was warmed to r.t. and stirred for 2 hours, concentrated, and purified by preparative HPLC (acetonitrile/water containing formic acid) to give compound 133 (23 mg, 19% yield). ESI-MS m/z: M2⁺ calcd. for C₁₀₆H₁₂₄F₂N₁₈O₂₂: 1019.46; found 1019.50.

### Example 166. Synthesis of 3-oxo-1-phenyl-2, 7, 10, 13, 16-pentaoxa-4-azanonadecan-19-oic acid (134)

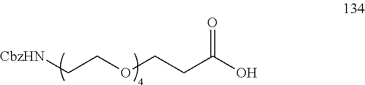

In a 500 mL flask, H₂N-PEG₄-CH₂CH₂CO₂H (3.0 g, 11.3 mmol, 1.0 eq.) and K₂CO₃ (4.7 g, 33.93 mmol, 3.0 eq.) were dissolved in 50 mL of water, and cooled over an ice water bath. CbzCl (2.50 g, 14.7 mmol, 1.3 eq.) in 50 mL of THF was added dropwise. The reaction was warmed to r.t. and stirred overnight. The reaction mixture was adjusted to pH 4-5 with 1N KHSO₄ and extracted with dichloromethane (200 mL×1, 100 mL×3), washed with water (500 mL), and brine (500 mL), dried over anhydrous sodium sulfate, and concentrated. The residue was dissolved in a small amount of dichloromethane and then loaded on a silica gel column, eluted with 24% MeOH/dichloromethane, and the fractions were combined and concentrated to give 3.8 g of colorless oil (yield 84%). ESI-MS m/z [M+H]⁺: calcd. for C₁₉H₂₉NO₈ 400.2, found: 400.2.

### Example 167. Synthesis of 2, 5-dioxopyrrolidin-1-yl 3-oxo-1-phenyl-2, 7, 10, 13, 16-pentaoxa-4-azanonadecan-19-oate (135)

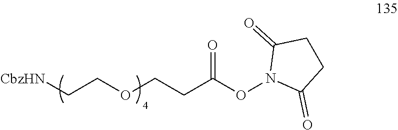

To a solution of CbzHN-PEG₄-CH₂CH₂CO₂H (3.8 g, 9.5 mmol, 1.0 eq.) in 50 mL of dry dichloromethane, N-hydroxysuccinimide (1.3 g, 11.4 mmol, 1.2 eq.) and EDC·HCl (9.1 g, 47.5 mmol, 5.0 eq.) were added. The reaction was stirred at r.t. overnight and then washed with water (50 mL×2), brine (100 mL×1), dried over anhydrous sodium sulfate, and concentrated. The crude product was used directly in the next step. ESI-MS m/z [M+H]⁺: calcd. for C₂₃H₃₂N₂O₁₀ 497.2, found: 497.2.

### Example 168. Synthesis of 3, 19-dioxo-1-phenyl-2, 7, 10, 13, 16, 23, 26, 29, 32-nonaoxa-4, 20-diazapentatriacontan-35-oic acid (136)

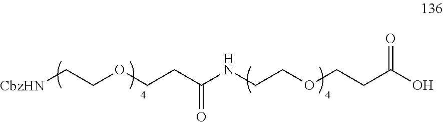

In a 300 mL flask, H₂N-PEG₄-CH₂CH₂CO₂H (2.6 g, 9.5 mmol, 1.0 eq.) and K₂CO₃ (3.9 g, 28.5 mmol, 3.0 eq.) were dissolved in 40 mL of water, cooled over an ice water bath, and the above crude N-hydroxysuccinimide ester solution (3.8 g, 9.5 mmol) in 40 mL of THF was added dropwise, and the mixture was warmed to r.t. and stirred overnight. The reaction mixture was adjusted to pH 4-5 using 1N KHSO₄, extracted with dichloromethane (150 mL×1, 100 mL×2), washed with water (200 mL), and brine (200 mL), dried over anhydrous sodium sulfate, and concentrated. The residue was dissolved in small amount of dichloromethane, and the loaded on a silica gel column, eluted with 4-6% MeOH/dichloromethane to give a colorless oil (4.91 g, 80% yield). ESI-MS m/z [M+H]⁺: calcd. for C₃₀H₅₀N₂O₁₃ 646.3, found: 646.3.

### Example 169. Synthesis of tert-butyl 3, 19, 35-trioxo-1-phenyl-2, 7, 10, 13, 16, 23, 26, 29, 32, 39, 42, 45, 48-tridecaoxa-4, 20, 36-triazahenpentacontan-51-oate (137)

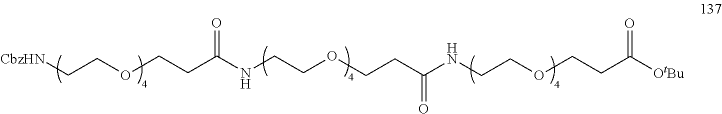

H₂N-PEG₄-CH₂CH₂CO₂^{t}Bu (0.48 g, 1.5 mmol, 1.0 eq.) was dissolved in 3 mL of DMF, cooled over ice/water bath, N, N-diisopropylethylamine (DIPEA) (0.78 g, 6.0 mmol, 4.0 eq.) was added dropwise, and followed by a solution of compound 136 (0.97 g, 1.5 mmol, 1.0 eq.) in 7 mL of DMF and HATU (1.72 g, 4.5 mmol, 3.0 eq.). The reaction was stirred over the ice bath for 2 hours, and diluted with 100 mL of water, extracted with dichloromethane (100 mL×3), washed with 1N KHSO₄ (200 mL), saturated sodium bicarbonate (200 mL), and brine (200 mL), dried over anhydrous sodium sulfate, and concentrated. The residue was dissolved in a small amount of dichloromethane, loaded on a silica gel column, and eluted 0-5% MeOH/dichloromethane. Fractions were combined and concentrated to give 1.22 g of light yellow oil (86% yield). ESI-MS m/z [M+H]⁺: calcd. for C₄₅H₇₉N₃O₁₈ 950.5, found: 950.5.

### Example 170. Synthesis of tert-butyl 1-amino-15, 31-dioxo-3, 6, 9, 12, 19, 22, 25, 28, 35, 38, 41, 44-dodecaoxa-16, 32-diazaheptatetracontan-47-oate (138)

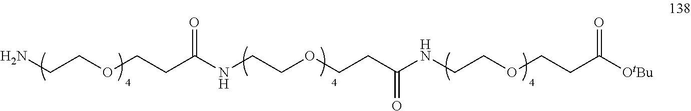

A solution of compound 137 (1.22 g, 1.28 mmol) in dichloromethane (5 mL) was stirred with Pd/C (5% wet, 500 mg) under 1 atm H₂ for 2 h. The reaction was then filtered over Celite and the filter cake was washed with MeOH. The filtrate and washings were combined and concentrated to give a light yellow oil (1.04 g, 100% yield). ESI-MS m/z [M+H]⁺: calcd. for C₃₇H₇₃N₃O₁₆ 816.5, found: 816.5.

### Example 171. Synthesis of (50R, 51R)-di-tert-butyl 50, 51-bis(((benzyloxy)carbonyl) amino)-17, 33, 49, 52, 68, 84-hexaoxo-4, 7, 10, 13, 20, 23, 26, 29, 36, 39, 42, 45, 56, 59, 62, 65, 72, 75, 78, 81, 88, 91, 94, 97-tetracosaoxa-16, 32, 48, 53, 69, 85-hexaazahectane-1, 100-dioate (139)

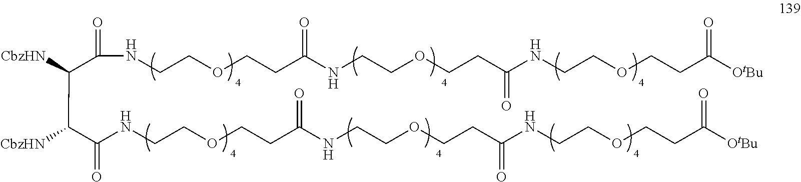

To a solution of compound 127 (0.26 g, 0.64 mmol) in DMA (10 mL) was added a solution of compound 138 (1.04 g, 1.28 mmol) in dichloromethane (5 mL), followed by DMAP (0.23 g, 1.92 mmol) and EDC·HCl (0.36 g, 1.92 mmol). The mixture was stirred overnight, concentrated and purified on silica gel column, eluted with MeOH/DCM (1:10) to afford compound 139 (0.81 g, 63% yield). ESI-MS m/z: [M+2H]²⁺ calcd. for C₉₄H₁₆₂N₈O₃₈ 1006.55; found 1006.70.

### Example 172. Synthesis of (50R, 51R)-di-tert-butyl 50, 51-diamino-17, 33, 49, 52, 68, 84-hexaoxo-4, 7, 10, 13, 20, 23, 26, 29, 36, 39, 42, 45, 56, 59, 62, 65, 72, 75, 78, 81, 88, 91, 94, 97-tetracosaoxa-16, 32, 48, 53, 69, 85-hexaazahectane-1, 100-dioate (140)

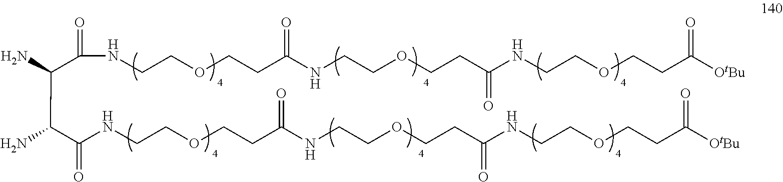

To a solution of compound 139 (0.81 g, 0.40 mmol) in MeOH (5 mL) was added 10% Pd/C (100 mg, 5 wt %), and the mixture was stirred under hydrogen atmosphere at r. t. for 18 h. Then the Pd/C catalyst was removed by filtration over Celite and the filter cake was washed with MeOH. The filtrate and washings were combined and concentrated to afford compound 140 (0.70 g, 100% yield). ESI-MS m/z: [M+2H]²⁺ calcd. for C₇₈H₁₅₀N₈O₃₄: 872.52; found 872.55.

### Example 173. Synthesis of (50R, 51R)-di-tert-butyl 50, 51-bis(4-(2, 5-dioxo-2, 5-dihydro-1H-pyrrol-1-yl)butanamido)-17, 33, 49, 52, 68, 84-hexaoxo-4, 7, 10, 13, 20, 23, 26, 29, 36, 39, 42, 45, 56, 59, 62, 65, 72, 75, 78, 81, 88, 91, 94, 97-tetracosaoxa-16, 32, 48, 53, 69, 85-hexaazahectane-1, 100-dioate (141) and (50S, 51S)-50, 51-bis(4-(2, 5-dioxo-2, 5-dihydro-1H-pyrrol-1-yl)butanamido)-17, 33, 49, 52, 68, 84-hexaoxo-4, 7, 10, 13, 20, 23, 26, 29, 36, 39, 42, 45, 56, 59, 62, 65, 72, 75, 78, 81, 88, 91, 94, 97-tetracosaoxa-16, 32, 48, 53, 69, 85-hexaazahectane-1, 100-dioic acid (142)

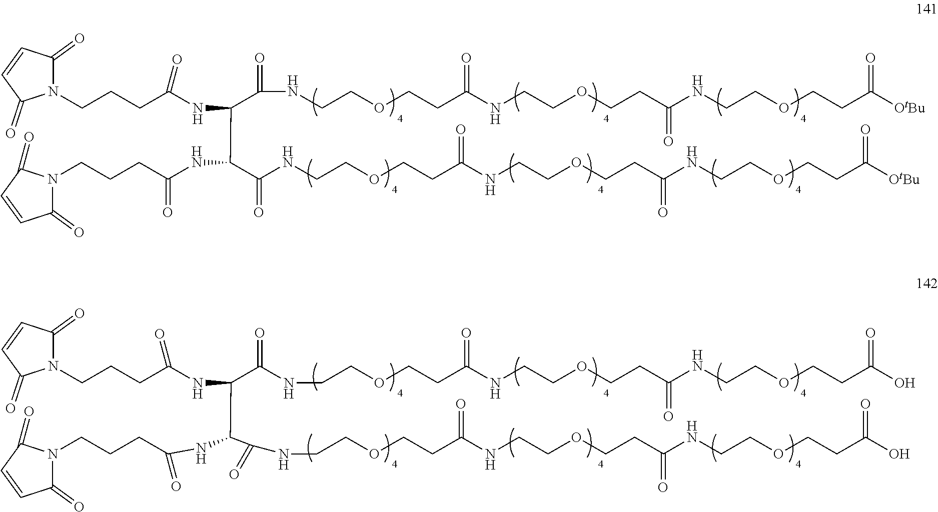

To a solution of 4-(2, 5-dioxo-2, 5-dihydro-1H-pyrrol-1-yl) butanoic acid (0.17 g, 1.00 mmol) and compound 140 (0.70 g, 0.40 mmol) in DMF (5 mL) were added N, N-diisopropylethylamine (0.88 mL, 5 mmol) and HATU (1.90 g, 12.56 mmol). The mixture was stirred overnight, concentrated and purified on silica gel column, eluted with 1-10% MeOH/DCM to afford compound 141 as an oil, (0.548 g, 66% yield). ESI-MS m/z [M+2H]²⁺. calcd. for C₉₄H₁₆₆N₁₀O₄₀ 2075.1264; found 2075.1350.

Compound 141 (0.54 g, 0.26 mmol) was dissolved in dichloromethane (5 mL) and treated with TFA (2.5 mL). The mixture was stirred at r.t. for 30 min, diluted with toluene (20 mL), concentrated to afford the title compound 142 (0.488, 96% yield) which was used for next step without further purification. ESI-MS m/z [M+H]: calcd. for C₈₆H₁₄₉N₁₀O₄₀ 1961.9933; found 1961.9987.

### Example 174. Synthesis of (S)—N, N′-(((((2S, 53S, 54S, 105S)-53, 54-bis(4-(2, 5-dioxo-2, 5-dihydro-1H-pyrrol-1-yl)butanamido)-2, 105-dimethyl-4, 20, 36, 52, 55, 71, 87, 103-octaoxo-7, 10, 13, 16, 23, 26, 29, 32, 39, 42, 45, 48, 59, 62, 65, 68, 75, 78, 81, 84, 91, 94, 97, 100-tetracosaoxa-3, 19, 35, 51, 56, 72, 88, 104-octaazahexahectane-1, 106-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (143)

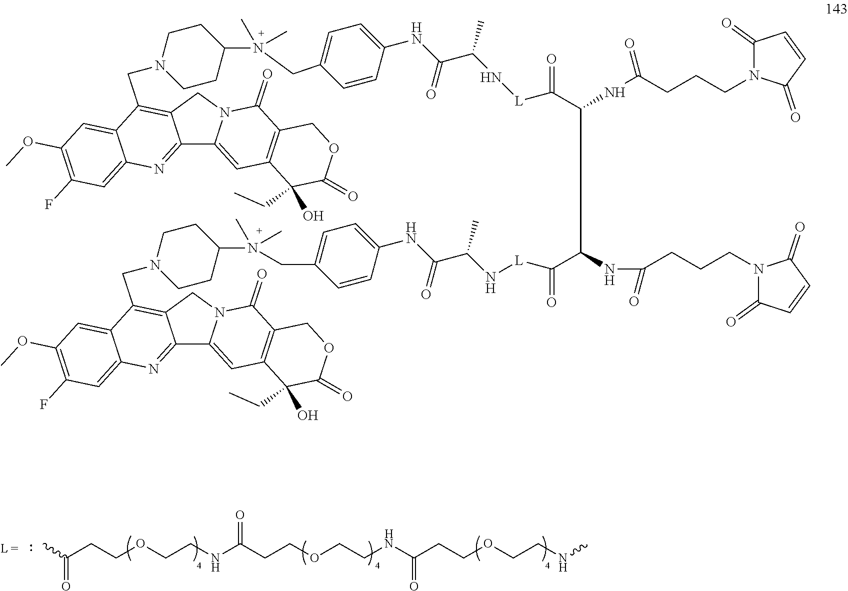

Compound 24 (47 mg, 0.060 mmol) and compound 142 (59 mg, 0.030 mmol) were dissolved in DMA (5 mL), cooled to about 0° C., and then EDC (23.1 mg, 0.12 mmol) and N, N-diisopropylethylamine (21 μL, 0.12 mmol) were added. The reaction was warmed to r.t. and stirred for 2 hours, concentrated, and purified by preparative HPLC (acetonitrile/water containing formic acid) to give compound 143 (36 mg, 36% yield). ESI-MS m/z: M2⁺ calcd. for C₁₆₄H₂₃₈F₂N₂₂O₅₀: 1675.8279; found 1675.8392.

### Example 175. Synthesis of 2, 5, 8, 11, 14, 17, 20, 23, 26-nonaoxaoctacosan-28-oic acid (144)

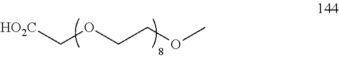

Tert-butyl 2, 5, 8, 11, 14, 17, 20, 23, 26-nonaoxaoctacosan-28-oate (210 g, 422 mmol) was dissolved in dichloromethane (400 mL) and anhydrous formic acid (1 L). The resulting solution was stirred at r.t. overnight. All volatiles were removed under vacuum, which afforded the title compound as a yellow oil (200 g, >100% yield).

### Example 176. Synthesis of 2, 5, 8, 11, 14, 17, 20, 23, 26-nonaoxaoctacosan-28-oyl chloride (145)

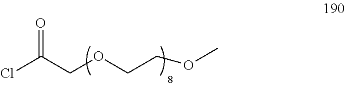

To the solution of 2, 5, 8, 11, 14, 17, 20, 23, 26-nonaoxaoctacosan-28-oic acid (198 g, 422 mmol) dissolved in dichloromethane (2.6 L), (COCl)₂ (275 mL) and DMF (0.5 mL) were added at r.t. The resulting solution was stirred at r.t. for 3 h. All volatiles were removed under vacuum to yield the title compound as a yellow oil (210 g, >100% yield).

### Example 177. Synthesis of (S)-34-(((benzyloxy)carbonyl)amino)-28-oxo-2, 5, 8, 11, 14, 17, 20, 23, 26-nonaoxa-29-azapentatriacontan-35-oic acid (146)

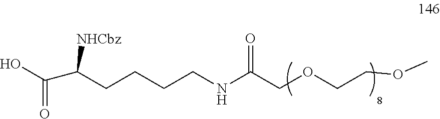

Z-L-Lys-OH (236 g, 844 mmol), Na₂CO₃ (89.5 g, 844 mmol) and NaOH (33.8 g, 844 mmol) were dissolved in water (1.6 L). The mixture was cooled under 0° C. using ice salt bath, to which a solution of 2, 5, 8, 11, 14, 17, 20, 23, 26-nonaoxaoctacosan-28-oyl chloride (210 g, 422 mmol) in THF (160 mL) was added. The resulting mixture was stirred at r.t. for 1 h, and then diluted with EtOAc (1 L). The aqueous layer was separated, to which concentrated HCl was added under ice cooling until pH 3 was reached. After extraction with dichloromethane, the organic layer was washed with brine, dried over Na₂SO₄ and concentrated to give the title compound as a yellow oil (290 g, 97% yield).

### Example 178. Synthesis of (S)-perfluorophenyl 34-(((benzyloxy)carbonyl)amino)-28-oxo-2, 5, 8, 11, 14, 17, 20, 23, 26-nonaoxa-29-azapentatriacontan-35-oate(147)

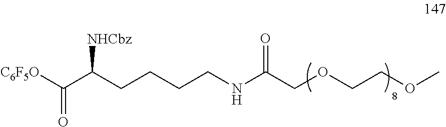

To a solution of compound 146 (183 g, 260 mmol) in dichloromethane (2 L) was added pentafluorophenol (95.4 g, 520 mmol) and DIC (131 g, 1.04 mol). The reaction was stirred at r.t. for 1 h, and then concentrated to give crude the title product (430 g).

### Example 179. Synthesis of (S)-tert-butyl 34-(((benzyloxy)carbonyl)amino)-28, 35-dioxo-2, 5, 8, 11, 14, 17, 20, 23, 26-nonaoxa-29, 36-diazatetracontan-40-oate (148)

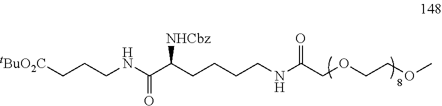

To a solution of tert-butyl 4-aminobutanoate (62.0 g, 390 mmol) in DMF (1.5 L) was added N, N-diisopropylethylamine (134 g, 1.04 mol) at 0° C. Compound 147 (430 g, crude) was then added at 10-20° C. and the resulting mixture was stirred at r.t. for 1 h. DMF was removed under vacuum and the residue was diluted with dichloromethane, washed with water. The aqueous phase was back-extracted with dichloromethane. The combined organic phase was washed with 0.2 N HCl and brine, dried over anhydrous Na₂SO₄, filtered and concentrated. Column chromatography (25% EtOAc/PE to pure EtOAc, then 0 to 5% MeOH/dichloromethane) gave the title compound as a yellow oil (180 g, 82% yield).

### Example 180. Synthesis of (S)-tert-butyl 34-amino-28, 35-dioxo-2, 5, 8, 11, 14, 17, 20, 23, 26-nonaoxa-29, 36-diazatetracontan-40-oate (149)

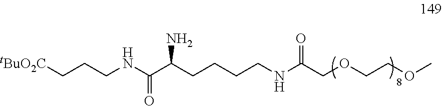

To a solution of compound 148 (78.0 g, 92.3 mmol, 1.0 eq.) in MeOH (500 mL) was added Pd/C (13 g, 10% Pd/C, 50% wet). The mixture was hydrogenated under 1 atm H₂ at r.t. overnight, then filtered and concentrated. The residue was purified by column chromatography (0 to 20% MeOH/dichloromethane) to give the title compound as a greenish yellow oil (70.2 g, 92% yield).

### Example 181. Synthesis of (S)-tert-butyl 34-(4-(2, 5-dioxo-2, 5-dihydro-1H-pyrrol-1-yl) butanamido)-28, 35-dioxo-2, 5, 8, 11, 14, 17, 20, 23, 26-nonaoxa-29, 36-diazatetracontan-40-oate (150)

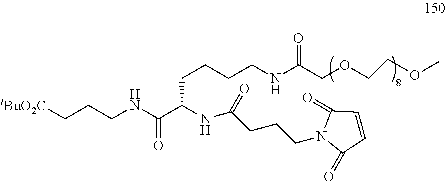

To a solution of (S)-tert-butyl 34-amino-28, 35-dioxo-2, 5, 8, 11, 14, 17, 20, 23, 26-nonaoxa-29, 36-diazatetracontan-40-oate (149, 0.93 g, 1.18 mmol) in 95% EtOH (50 mL) and NaH₂PO₄ solution (0.1 M, pH 5.0, 10 mL), N-succinimidyl 4-maleimido-butyrate (0.50 g, 1.77 mmol, 1.5 eq.) was added. The mixture was stirred overnight, then concentrated and diluted with water (50 mL) and extracted with dichloromethane (80 mL×3), dried over anhydrous sodium sulfate, filtered, concentrated and purified by silica gel column chromatography (25:1 dichloromethane/methanol) to give the title compound as a light yellow oil (0.82 g, 80% yield). ESI MS m/z 877.52 ([M+H]⁺).

### Example 182. Synthesis of (S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontan-40-oic acid (151)

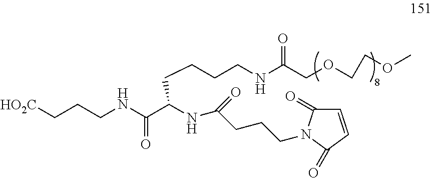

(S)-tert-butyl 34-(4-(2, 5-dioxo-2, 5-dihydro-1H-pyrrol-1-yl) butanamido)-28, 35-dioxo-2, 5, 8, 11, 14, 17, 20, 23, 26-nonaoxa-29, 36-diazatetracontan-40-oate (0.82 g, 0.94 mmol) was dissolved in HCOOH (50 mL) and stirred at room temperature for 1 hour. The reaction mixture was concentrated and co-evaporated with toluene twice, and the residue was placed on a vacuum pump to give the title compound (0.80 g, crude product). ESI MS m/z 820.45 ([M+H]⁺).

### Example 183. Synthesis of (S)-2, 5-dioxopyrrolidin-1-yl 34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontan-40-oate (152)

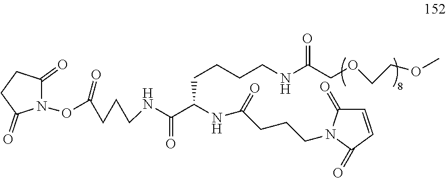

To a solution of (S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontan-40-oic acid (0.80 g, crude, 0.94 mmol) in DMA (5.0 mL), NHS (0.12 g, 1.03 mmol) and EDC·HCl (0.27 g, 1.41 mmol) were added, and the reaction was stirred at r.t. for 2 h, then diluted with water (15 mL) and extracted with ethyl acetate (3×10 mL). The combined organic phase was washed with brine (10 mL), dried over anhydrous sodium sulfate, filtered and concentrated. The residue was purified by silica gel column (10-50% ethyl acetate/petroleum ether) to give a colorless oil compound (0.67 g, 78% yield). ESI MS m/z 918.55 ([M+H]⁺).

### Example 184. Synthesis of (7S, 10R, 11R, 14S)-di-tert-butyl 10, 11-bis(((benzyloxy) carbonyl)amino)-6, 9, 12, 15-tetraoxo-7, 14-bis(28-oxo-2, 5, 8, 11, 14, 17, 20, 23, 26-nonaoxa-29-azatritriacontan-33-yl)-5, 8, 13, 16-tetraazaicosane-1, 20-dioate (153)

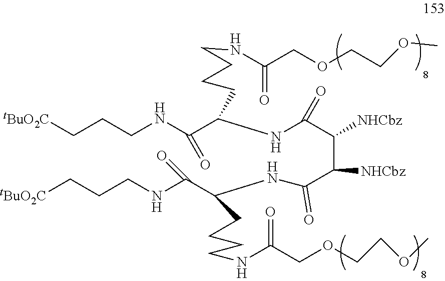

To a solution of compound 127 (0.85 g, 2.00 mmol) in DMA (10 mL) were added a solution of compound 149 (3.20 g, 4.50 mmol) in dichloromethane (10 mL), DMAP (1.50 g, 12 mmol) and EDC·HCl (2.3 g, 12 mmol). The mixture was stirred overnight, concentrated and purified on silica gel column, eluted with EtOAc/DCM (1:10) to afford compound 153 (3.33 g, 88% yield). ESI-MS m/z: [M+2H]²⁺ calcd. for C₈₆H₁₄₆N₈O₃₂ 902.50; found 902.55.

### Example 185. Synthesis of (7S, 10R, 1IR, 14S)-di-tert-butyl 10, 11-bis(4-(2, 5-dioxo-2, 5-dihydro-1H-pyrrol-1-yl)butanamido)-6, 9, 12, 15-tetraoxo-7, 14-bis(28-oxo-2, 5, 8, 11, 14, 17, 20, 23, 26-nonaoxa-29-azatritriacontan-33-yl)-5, 8, 13, 16-tetraazaicosane-1, 20-dioate (154)

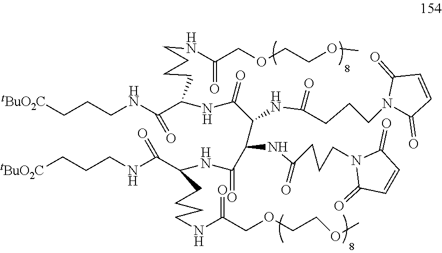

A mixture of compound 153 (3.33 g, 1.76 mmol) and Pd/C (5 wt %, 0.10 g) in dichloromethane (50 mL) was hydrogenated under 1 atm H₂ pressure overnight and then filtered over Celite (filter aid). The filtrate was concentrated and then dissolved in DMF (10 mL), to which EDC·HCl (1.00 g, 5.28 mmol) and 4-(2,5-dioxo-2,5-dihydro-1H-pyrrol-1-yl)butanoic acid (1.84 g, 5.28 mmol) were added. The mixture was stirred at r. t. for 16 h, concentrated and purified by silica gel column chromatography (1:4 MeOH/dichloromethane) to give an oil (2.56 g, 78% yield). ESI-MS m/z: [M+2H]2⁺ calcd. for C₈₆H₁₄₈N₁₀O₃₄ 933.51; found 933.55.

### Example 186. Synthesis of (S)—N, N′-(((((2S, 10S, 13R, 14R, 17S, 25S)-13, 14-bis(4-(2, 5-dioxo-2, 5-dihydro-1H-pyrrol-1-yl)butanamido)-2, 25-dimethyl-4, 9, 12, 15, 18, 23-hexaoxo-10, 17-bis(28-oxo-2, 5, 8, 11, 14, 17, 20, 23, 26-nonaoxa-29-azatritriacontan-33-yl)-3, 8, 11, 16, 19, 24-hexaazahexa-cosane-1, 26-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) formic acid salt (155)

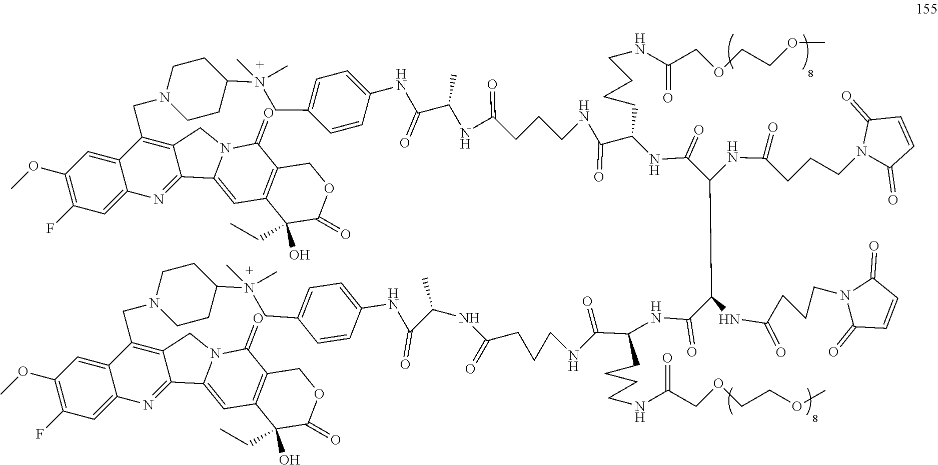

A mixture of compound 154 (1.00 g, 0.536 mmol) in dichloromethane (5 mL) and formic acid (5 mL) was stirred at r.t. for 24 h, and then concentrated. The residue was dissolved in DMA (5 mL), to which compound 24 (0.64 g, 0.89 mmol), triethylamine (0.15 mL, 1.07 mmol) and HATU (0.41 g, 1.07 mmol) were added and stirred at r.t. for 16 h. After the solvent was removed under high vacuum, the residue was purified by preparative HPLC (acetonitrile/water containing formic acid) (acetonitrile/water) to afford the title compound 155 (1.06 g, 63% yield). ESI-MS m/z: M2⁺ calcd. for C₁₅₆H₂₂₀F₂N₂₂O₄₄ 1571.78; found 1571.78.

### Example 187. Synthesis of methyl 4-(bis(2-hydroxyethyl)amino)-4-oxobutanoate (156)

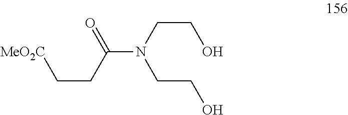 [SMILES: COC(=O)CCC(=O)N(CCO)CCO]

Dimethyl succinate (20.0 g, 136.9 mmol) and dihydroxyethylamine (7.20 g, 68.7 mmol) in a mixture of anhydrous toluene (500 mL) and pyridine (50 mL) were heated at 150° C. for 28 h. The mixture was concentrated and purified on silica gel column eluted with 5-25% ethyl acetate/dichloromethane to afford the title compound (12.5 g, 83% yield). ESI-MS m/z 242.42 ([M+Na]⁺).

### Example 188. Synthesis of methyl 4-(bis(2-((methylsulfonyl)oxy)ethyl) amino)-4-oxobutanoate (157)

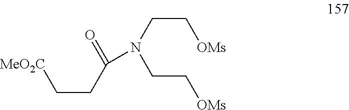 [SMILES: COC(=O)CCC(=O)N(CCOS(C)(=O)=O)CCOS(C)(=O)=O]

To a solution of methyl 4-(bis(2-hydroxyethyl)amino)-4-oxobutanoate (12.0 g, 49.56 mmol) in anhydrous pyridine (350 mL), methanesulfonyl chloride (20.0 g, 175.4 mmol) was added. After stirring overnight, the mixture was concentrated, diluted with ethyl acetate (350 mL), washed with cold 1 M NaH₂PO₄ (2×300 mL), dried over Na₂SO₄, filtered and evaporated to afford crude product (˜18.8 g, >100% yield). The crude product was used in the next step without further purification. ESI-MS m/z 376.06 ([M+H]⁺).

### Example 189. Synthesis of 3, 6-endoxo-Δ-tetrahydrophthalimide (158)

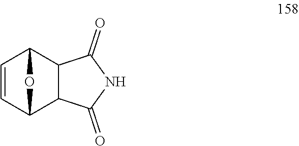 [SMILES: O=C1NC(=O)C2C1[C@@H]1C=C[C@H]2O1]

To a solution of maleimide (10.0 g, 103.0 mmol) in toluene (200 mL) was added furan (10.0 mL, 137.4 mmol). The mixture was heated in a 1 L auto Clave bomb at 100° C. for 8 h. The bomb was cooled to r. t., and the solid was rinsed out with MeOH, concentrated and crystallized in ethyl acetate/hexane to afford 16.7 g (99%) of the title compound. 1H NMR (CDCl₃): 11.12 (s, 1H), 6.68˜6.64 (m, 2H), 5.18˜5.13 (m, 2H), 2.97˜2.92 (m, 2H); ESI-MS m/z 188.04 ([M+Na]⁺).

### Example 190. Synthesis of Methyl 4-((2-((3aR, 4R, 7S, 7aS)-1, 3-dioxo-3a, 4, 7, 7a-tetrahydro-1H-4, 7-epoxyisoindol-2(3H)-yl)ethyl)(2-((4R, 7S, 7aS)-1, 3-dioxo-3a, 4, 7, 7a-tetrahydro-1H-4, 7-epoxyisoindol-2(3H)-yl)ethyl)amino)-4-oxobutanoate (159)

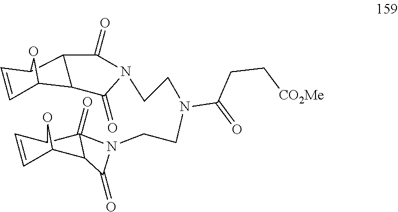

To a solution of methyl 4-(bis(2-((methylsulfonyl)oxy)ethyl)amino)-4-oxobutanoate (157, fresh made, 90% pure, 8.5 g, ˜20 mmol) in DMA (350 mL), 3, 6-endoxo-A-tetrahydrophthalimide (158, 10.2 g, 61.8 mmol), sodium carbonate (8.0 g, 75.5 mmol) and sodium iodide (0.3 g, 2.0 mmol) were added. The mixture was stirred at r. t. overnight, concentrated, diluted with ethyl acetate (350 mL), washed with sat'ed NaHCO₃ solution (300 mL), brine (300 mL) and 1 M NaH₂PO₄ (300 mL). The organic layer was dried over sodium sulfate, filtered, evaporated, loaded on silica gel column and eluted with 10-30% ethyl acetate/hexane to afford the title compound (7.9 g, 77% yield). ESI-MS m/z 536.4 ([M+Na]⁺).

### Example 191. Synthesis of 4-(bis(2-(2, 5-dioxo-2, 5-dihydro-1H-pyrrol-1-yl)ethyl) amino)-4-oxobutanoic acid (160)

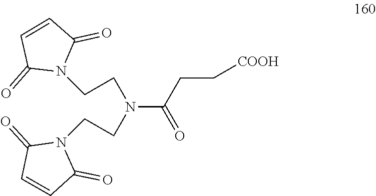 [SMILES: O=C(O)CCC(=O)N(CCN1C(=O)C=CC1=O)CCN1C(=O)C=CC1=O]

Compound 159 (3.0 g, 5.8 mmol) and trimethylstannanol (4.8 g, 26.4 mmol) in 1, 2-dichloroethane (150 mL) were refluxed at 80° C. for 8 h, then cooled to r. t. and the residue was passed through a short silica gel column and eluted with dichloromethane/MeOH to remove excess trimethyltin hydroxide. Then the pooled fractions were combined, concentrated and diluted with DMA and toluene, heated to 120° C. and stirred overnight. The reaction mixture was loaded on silica gel column and eluted with 5-10% MeOH/dichloromethane to afford the title compound (1.62 g, 76% yield). ESI-MS m/z 386.2 ([M+Na]⁺).

### Example 192. Synthesis of N-(methoxycarbonyl)maleimide (161)

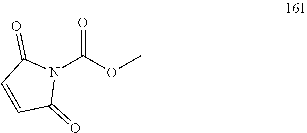

Maleimide (12.0 g, 123.7 mmol) was dissolved in ethyl acetate (150 mL) in a 250 mL round-bottom flask, and the solution was cooled to approximately 0° C. A solution of N-methyl morpholine (14.1 mL, 12.8 g, 126.2 mmol) in ethyl acetate (10 mL) was added dropwise over 15 min. A solution of methyl chloroformate (9.60 mL, 11.5 g, 123.7 mmol) in ethyl acetate (50 mL) was added dropwise, and the solution was warmed to room temperature and stirring for 2 h. The solution was diluted with ethyl acetate (100 mL) and washed with saturated aqueous sodium bicarbonate solution, water, and saturated sodium chloride solution. The organic layer was separated, dried over Na₂SO₄, and filtered. The supernatant was concentrated under reduced pressure to yield the title compound as a solid (15.9 g, 102.5 mmol, 82.9% yield). ¹H NMR (500 MHz, CDCl₃): δ 6.84 (s, 2H), 3.97 (s, 3H).

### Example 193. Synthesis of tert-butyl bis(2-(2, 5-dioxo-2, 5-dihydro-1H-pyrrol-1-yl)ethyl)carbamate (162)

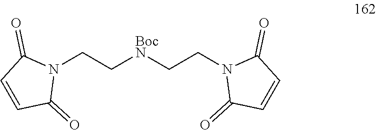 [SMILES: CC(C)(C)OC(=O)N(CCN1C(=O)C=CC1=O)CCN1C(=O)C=CC1=O]

Tert-butyl bis(2-aminoethyl)carbamate (4.00 g, 19.68 mmol) was dissolved in a mixture of saturated solution of NaHCO₃ (80 mL) and MeOH (10 mL) cooled at 0° C. N-(methoxycarbonyl)maleimide (6.20 g, 40.00 mmol) was added to the stirred solution. After 20 mins the reaction mixture was diluted with water (150 mL) and stirred for 30 min at room temperature. The reaction mixture was cooled to 0° C., and the reaction mixture was filtered and washed with ice-cold water (100 mL). Drying in high vacuum afforded the title compound (5.51 g, 77.1% yield) as a white solid. ESI MS m/z C₁₇H₂₂N₃O₆ [M+H]⁺, calcd. 363.15, found 364.20.

### Example 194. Synthesis of 1, 1′-(azanediylbis(ethane-2, 1-diyl))bis(1H-pyrrole-2, 5-dione), HCl salt (163)

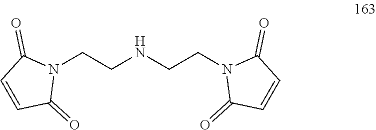 [SMILES: O=C1C=CC(=O)N1CCNCCN1C(=O)C=CC1=O]

Tert-butyl bis(2-(2, 5-dioxo-2, 5-dihydro-1H-pyrrol-1-yl)ethyl)carbamate (5.50 g, 15.14 mmol) in dioxane (40 mL) at 0° C. was added HCl (37% conc, 10 mL). The mixture was stirred on the ice bath for 30 min, evaporated, concentrated and coevaporated with dioxane/toluene (1:1, 3×40 mL) and dried in high vacuum to afford the title compound (4.40 g, 97%) which was used for the next step without further purification. ESI MS m/z C₁₂H₁₄N₃O₄ [M+H]⁺, calcd. 264.09, found 264.20.

### Example 195. Synthesis of 2-(2-(bis(2-(2, 5-dioxo-2, 5-dihydro-1H-pyrrol-1-yl)ethyl)amino)-2-oxoethoxy)acetic acid (164)

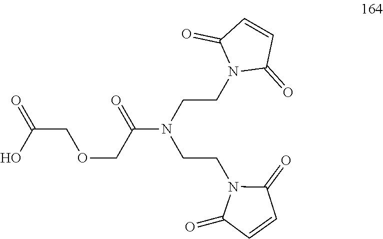 [SMILES: O=C(O)COCC(=O)N(CCN1C(=O)C=CC1=O)CCN1C(=O)C=CC1=O]

1, 1′-(azanediylbis(ethane-2, 1-diyl))bis(1H-pyrrole-2, 5-dione), HCl salt (2.01 g, 6.70 mmol) in the mixture of ethanol (50 mL) and NaH₂PO₄ buffer (100 mL, 100 mM, pH 7.0) on a ice bath was added 1, 4-dioxane-2, 6-dione (0.80 g, 6.89 mmol). The mixture then was stirred at r.t. (room temperature) for 4 h, concentrated, purified on silica gel column eluted with H₂O/CH₃CN (1:99 to 3:97) to afford the title compound (2.16 g, 85% yield). ESI MS m/z C₁₆H₁₈N₃O₈ [M+H]⁺, calcd. 380.11, found 380.20.

### Example 196. Synthesis of 4-(bis(2-(2, 5-dioxo-2, 5-dihydro-1H-pyrrol-1-yl)ethyl)amino)-4-oxobutanoic acid (165)

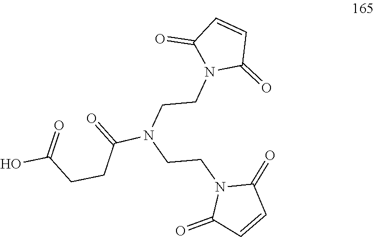 [SMILES: O=C(O)CCC(=O)N(CCN1C(=O)C=CC1=O)CCN1C(=O)C=CC1=O]

1, 1′-(azanediylbis(ethane-2, 1-diyl))bis(1H-pyrrole-2, 5-dione), HCl salt (2.01 g, 6.70 mmol) in the mixture of ethanol (50 mL) and NaH₂PO₄ buffer (100 mL, 100 mM, pH 7.0) on a ice bath was added dihydrofuran-2, 5-dione (0.68 g, 6.80 mmol). The mixture then was stirred at r.t. (room temperature) for 4 h, concentrated, purified on silica gel column eluted with H₂O/CH₃CN (100% CH₃CN to 3% H₂O in CH₃CN) to afford the title compound (2.09 g, 86% yield). ESI MS m/z C₁₆H₁₈N₃O₇ [M+H]⁺, calcd. 364.11, found 364.20.

### Example 197. Synthesis of 2, 5-dioxopyrrolidin-1-yl 2-(2-(bis(2-(2, 5-dioxo-2, 5-dihydro-1H-pyrrol-1-yl)ethyl)amino)-2-oxoethoxy)acetate (166)

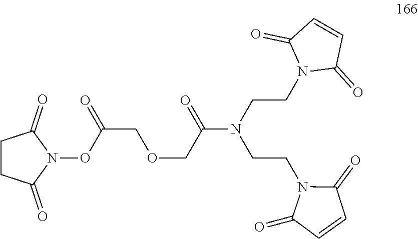 [SMILES: O=C(COCC(=O)N(CCN1C(=O)C=CC1=O)CCN1C(=O)C=CC1=O)ON1C(=O)CCC1=O]

In the mixture solution of 2-(2-(bis(2-(2, 5-dioxo-2, 5-dihydro-1H-pyrrol-1-yl)ethyl)amino)-2-oxoethoxy)acetic acid (1.10 g, 2.90 mmol) and N-hydroxysuccinimide (0.36 g, 3.12 mmol) in dry DMA (40 mL) was added EDC (1.20 g, 6.25 mmol). The reaction mixture was stirred for 4 h, then concentrated and purified by silica gel column chromatography (10:1 to 5:1 DCM/EtOAc) to give the title compound (1.09 g, 79% yield). ESI MS m/z: calcd. for C₂₀H₂₁N₄O₁₀ [M+H]⁺ 477.12, found 477.20.

### Example 198. Synthesis of 2, 5-dioxopyrrolidin-1-yl 4-(bis(2-(2, 5-dioxo-2, 5-dihydro-1H-pyrrol-1-yl)ethyl)amino)-4-oxobutanoate (167)

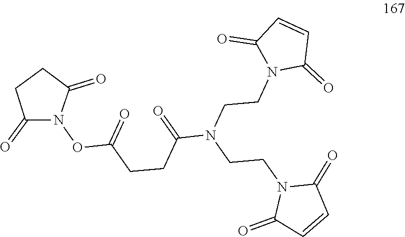 [SMILES: O=C(CCC(=O)N(CCN1C(=O)C=CC1=O)CCN1C(=O)C=CC1=O)ON1C(=O)CCC1=O]

In the mixture solution of 4-(bis(2-(2, 5-dioxo-2, 5-dihydro-1H-pyrrol-1-yl)ethyl)amino)-4-oxobutanoic acid (1.05 g, 2.89 mmol) and N-hydroxysuccinimide (0.36 g, 3.12 mmol) in dry DMA (40 mL) was added EDC (1.20 g, 6.25 mmol). The reaction mixture was stirred for 4 h, then concentrated and purified by silica gel column chromatography (10:1 to 5:1 DCM/EtOAc) to give the title compound (1.10 g, 83% yield). ESI MS m/z: calcd. for C₂₀H₂₁N₄O₉ [M+H]⁺ 461.12, found 461.20

### Example 199. Synthesis of N-(4-((S)-2-(4-(bis(2-(2, 5-dioxo-2, 5-dihydro-1H-pyrrol-1-yl)ethyl)amino)-4-oxobutanamido)propanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium (168)

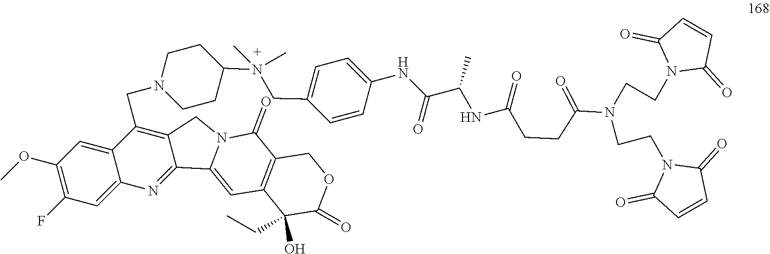

The crude product from the previous step (0.20 g) was dissolved in DMA (5 mL), to which compound 24 (0.71 g, 1.00 mmol) and N, N-diisopropylethylamine (0.20 mL, 1.20 mmol) were added at 0° C. The reaction was warmed to r.t. and stirred for 2 hours, concentrated, and purified by preparative HPLC (acetonitrile/water containing formic acid) to give compound 168 (0.85 g, 80% yield). ESI-MS m/z: M⁺ calcd. for C₅₅H₆₁FN₉O₁₂: 1058.44; found 1058.60.

### Example 200. Synthesis of (S)-tert-butyl 34-(4-(bis(2-(2, 5-dioxo-2, 5-dihydro-1H-pyrrol-1-yl)ethyl)amino)-4-oxobutanamido)-28, 35-dioxo-2, 5, 8, 11, 14, 17, 20, 23, 26-nonaoxa-29, 36-diazatetracontan-40-oate (169)

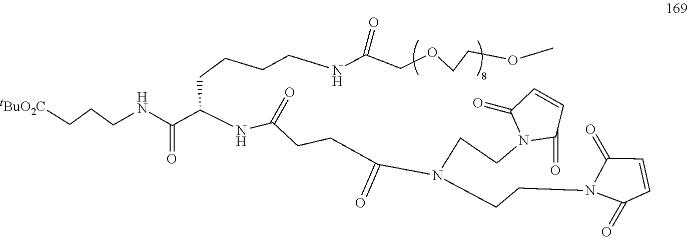

To a solution of compound 149 (2.98 g, 4.20 mmol) and compound 165 (1.39 g, 3.82 mmol) in DMA (20 mL), EDC·HCl (0.80 g, 4.20 mmol) was added. The reaction was stirred at r.t. overnight, then poured onto water (50 mL) and extracted with ethyl acetate (3×40 mL). The combined organic phase was washed with brine (40 mL), dried over anhydrous sodium sulfate, filtered and concentrated. The residue was purified by column chromatography (10-50% ethyl acetate/petroleum ether) to give a colorless oil (3.23 g, 80% yield). ESI-MS m/z 1057.85 ([M+H]⁺).

### Example 201. Synthesis of (S)-34-(4-(bis(2-(2, 5-dioxo-2, 5-dihydro-1H-pyrrol-1-yl)ethyl)amino)-4-oxobutanamido)-28, 35-dioxo-2, 5, 8, 11, 14, 17, 20, 23, 26-nonaoxa-29, 36-diazatetracontan-40-oic acid (170)

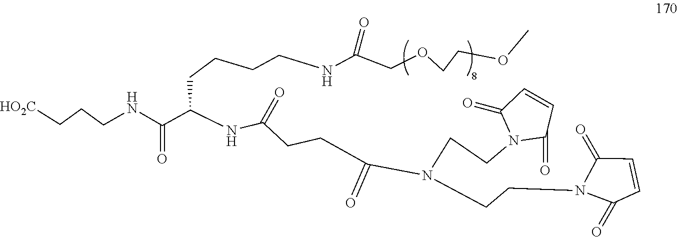

A solution of compound 169 (3.20 g, 3.03 mmol) in formic acid (10 mL) and dichloromethane (5 mL) was stirred at r.t. overnight. The solution was then concentrated and co-evaporated with toluene three times to give a colorless oil (3.00 g, crude), which was used without further purification. ESI-MS m/z 1001.50 ([M+H]⁺).

### Example 202. Synthesis of (S)-2, 5-dioxopyrrolidin-1-yl 34-(4-(bis(2-(2, 5-dioxo-2, 5-dihydro-1H-pyrrol-1-yl)ethyl)amino)-4-oxobutanamido)-28, 35-dioxo-2, 5, 8, 11, 14, 17, 20, 23, 26-nonaoxa-29, 36-diazatetracontan-40-oate (171)

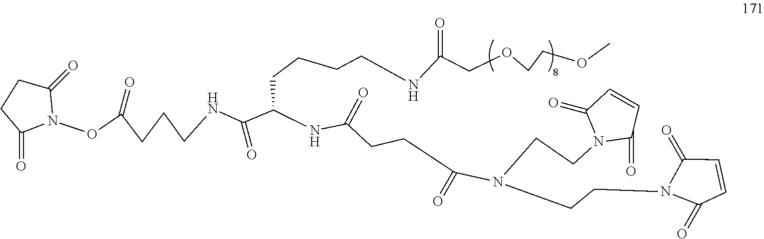

To a solution of compound 170 (3.00 g, crude, 3.03 mmol) in DMA (15.0 mL), N-hydroxysuccinimide (0.38 g, 3.33 mmol) and EDC·HCl (0.87 g, 4.55 mmol) were added, and the reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil (2.90 g, 90% yield). ESI-MS m/z 1098.50 ([M+H]⁺).

### Example 203. Synthesis of N-(4-((34S, 42S)-34-(4-(bis(2-(2, 5-dioxo-2, 5-dihydro-1H-pyrrol-1-yl)ethyl)amino)-4-oxobutanamido)-42-methyl-28, 35, 40-trioxo-2, 5, 8, 11, 14, 17, 20, 23, 26-nonaoxa-29, 36, 41-triazatritetracontanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium (172)

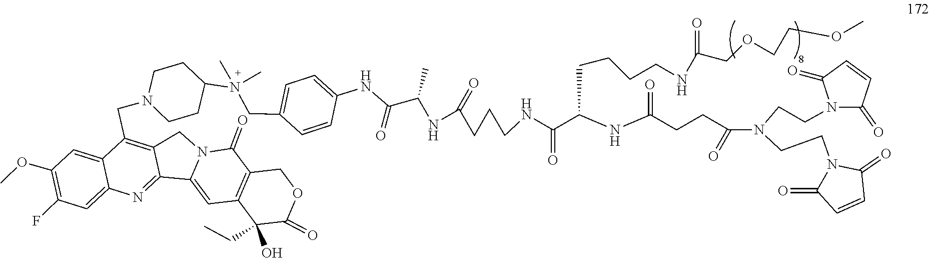

Compound 171 (0.10 g, 0.091 mmol) was dissolved in DMA (5 mL), to which compound 24 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 172 (84 mg, 62% yield). ESI-MS m/z: M⁺ calcd. for C₈₄H₁₁₆FN₁₂O₂₄: 1695.82; found 1695.82.

### Example 204. Synthesis of tert-butyl 2-(2-(1, 3-dioxoisoindolin-2-yl)acetyl) hydrazinecarboxylate (173)

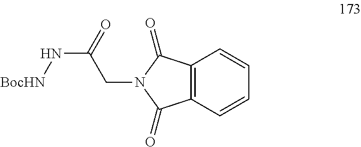 [SMILES: CC(C)(C)OC(=O)NNC(=O)CN1C(=O)c2ccccc2C1=O]

To a solution of Boc-hydrazine (7.08. g, 53.5 mmol) in dichloromethane (200 mL) at 0° C., triethylamine (13.5 mL, 97.4 mmol) and 2-(1,3-dioxoisoindolin-2-yl)acetyl chloride (10.8 g, 48.7 mmol) was added in sequence. After stirred at r.t. for 30 min, the mixture was poured into ice-water (100 mL) and extracted with dichloromethane (3×100 mL). The combined organic phases were washed with water (100 mL) and brine (100 mL), dried over anhydrous sodium sulfate, filtered and concentrated to give a white solid (15.5 g, 100% yield). ESI-MS m/z 320.12 ([M+H]⁺).

### Example 205. Synthesis of 2-(1, 3-dioxoisoindolin-2-yl)acetohydrazide (174)

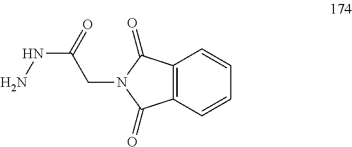 [SMILES: NNC(=O)CN1C(=O)c2ccccc2C1=O]

Compound 173 (15.5 g, 48.7 mmol) was dissolved in 1, 4-dioxane (150 mL) and treated with 25% HCl (50 mL) at r.t. for 1 h. The reaction mixture was concentrated and then co-evaporated with toluene to give a white solid (10.6 g, 100% yield). ESI-MS m/z 220.06 ([M+H]⁺).

### Example 206. Synthesis of 2-(1, 3-dioxoisoindolin-2-yl)-N′-(2-(1, 3-dioxoisoindolin-2-yl)acetyl)acetohydrazide (175)

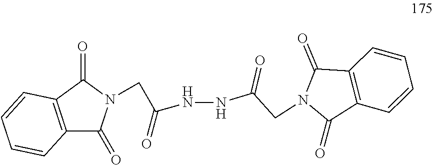 [SMILES: O=C(CN1C(=O)c2ccccc2C1=O)NNC(=O)CN1C(=O)c2ccccc2C1=O]

To a solution of compound 174 (10.6 g, 48.7 mmol) in THF (200 mL) at 0° C., triethylamine (13.5 mL, 97.4 mmol) and 2-(1,3-dioxoisoindolin-2-yl)acetyl chloride (10.8 g, 48.7 mmol) were added. The reaction was warmed to r.t. and stirred overnight. The precipitate was collected by filtration and suspended in water (100 mL) and stirred for 20 min. The mixture was filtered again and a white solid (15.7 g, 80% yield) was collected as compound 175. ESI-MS m/z 407.09 ([M+H]⁺).

### Example 207. Synthesis of di-tert-butyl 2, 2′-(1, 2-bis(2-(1, 3-dioxoisoindolin-2-yl)acetyl)hydrazine-1, 2-diyl)diacetate (176)

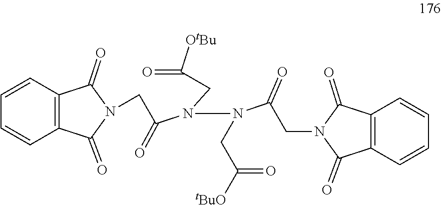 [SMILES: CC(C)(C)OC(=O)CN(C(=O)CN1C(=O)c2ccccc2C1=O)N(CC(=O)OC(C)(C)C)C(=O)CN1C(=O)c2ccccc2C1=O]

NaH (0.5 g, 12.3 mmol) was added to a solution of compound 175 (2.0 g, 4.92 mmol) in DMF (40 mL) at 0° C. in portions. The mixture was warmed to r.t. and stirred for 3 h. After that tert-butyl bromoacetate (2.0 g, 10.3 mmol) was added and the reaction was stirred overnight before pouring into ice-water (100 mL) and extracting with dichloromethane (3×50 mL). The combined organic phase was washed with water (50 mL), brine (50 mL), dried over anhydrous sodium sulfate, filtered and concentrated, purified by silica gel chromatography to give a white solid (1.5 g, 50% yield). ESI-MS m/z 635.23 ([M+H]⁺).

### Example 208. Synthesis of di-tert-butyl 2, 2′-(1, 2-bis(2-aminoacetyl)hydrazine-1, 2-diyl)diacetate (177)

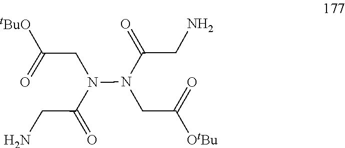 [SMILES: CC(C)(C)OC(=O)CN(C(=O)CN)N(CC(=O)OC(C)(C)C)C(=O)CN]

A mixture of compound 176 (1.5 g, 2.36 mmol) and hydrazine (442 mg, 7.08 mmol) in ethanol (30 mL) was refluxed for 1 h, then cooled to r.t. and filtered. The filtrate was concentrated and taken up in ethyl acetate (20 mL), filtered again. The filtrate was concentrated to give a white solid 177 (750 mg, 85% yield). ESI-MS m/z 375.22 ([M+H]⁺).

### Example 209. Synthesis of di-tert-butyl 2, 2′-(1, 2-bis(2-(2, 5-dioxo-2, 5-dihydro-1H-pyrrol-1-yl)acetyl)hydrazine-1, 2-diyl)diacetate (178)

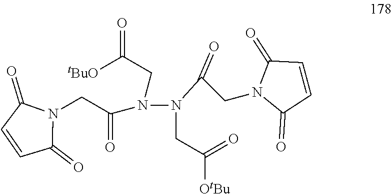 [SMILES: CC(C)(C)OC(=O)CN(C(=O)CN1C(=O)C=CC1=O)N(CC(=O)OC(C)(C)C)C(=O)CN1C(=O)C=CC1=O]

A solution of compound 177 (750 mg, 2 mmol) in THF (20 mL) and saturated NaHCO₃ aqueous solution (30 mL) at 0° C., N-methoxycarbonyl maleimide (622 mg, 4 mmol) was added. The reaction mixture was stirred at 0° C. for 1 h. A white solid was collected by filtration as compound 178 (854 mg, 80% yield). ESI-MS m/z 535.20 ([M+H]⁺).

### Example 210. Synthesis of 2, 2′-(1, 2-bis(2-(2, 5-dioxo-2, 5-dihydro-1H-pyrrol-1-yl)acetyl)hydrazine-1, 2-diyl)diacetic acid (179)

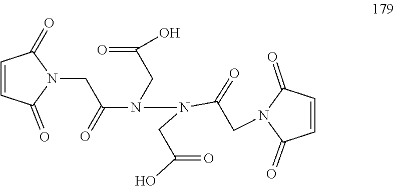 [SMILES: O=C(O)CN(C(=O)CN1C(=O)C=CC1=O)N(CC(=O)O)C(=O)CN1C(=O)C=CC1=O]

Compound 178 (854 mg, 1.6 mmol) was dissolved in dioxane (3 mL) and treated with 25% HCl (3 mL) at r.t. for 2 h. The reaction was then evaporated to give compound 179 (675 mg, 100% yield). ESI-MS m/z 423.07 ([M+H]⁺).

### Example 211. Synthesis of di-tert-butyl 4, 4′-((2, 2′-(1, 2-bis(2-(2, 5-dioxo-2, 5-dihydro-1H-pyrrol-1-yl)acetyl)hydrazine-1, 2-diyl)bis(acetyl))bis(azanediyl))dibutanoate (180)

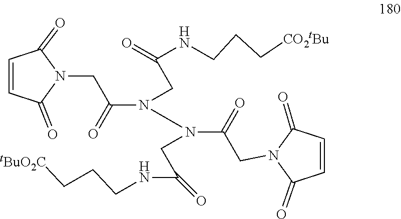 [SMILES: CC(C)(C)OC(=O)CCCNC(=O)CN(C(=O)CN1C(=O)C=CC1=O)N(CC(=O)NCCCC(=O)OC(C)(C)C)C(=O)CN1C(=O)C=CC1=O]

To a solution of compound 179 (200 mg, 0.47 mmol) in DMF (5 mL) at 0° C., tert-butyl 4-aminobutanoate (158 mg, 0.99 mmol) and EDC·HCl (189.7 mg, 0.99 mmol) were added. The reaction mixture was warmed to r.t. and stirred overnight, poured into ice-water, and extraction with dichloromethane (3×10 mL). The combined organic phase was washed with 0.2 N HCl (5 mL), water (5 mL), brine (5 mL), dried over anhydrous sodium sulfate, filtered and concentrated to give a white solid (330 mg, 100% yield).

### Example 212. Synthesis of bis(2, 5-dioxopyrrolidin-1-yl) 4, 4′-((2, 2′-(1, 2-bis(2-(2, 5-dioxo-2, 5-dihydro-1H-pyrrol-1-yl)acetyl)hydrazine-1, 2-diyl)bis(acetyl)) bis(azanediyl))dibutanoate (181)

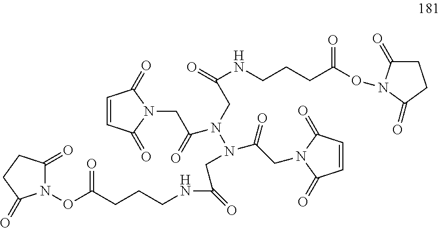 [SMILES: O=C(CN(C(=O)CN1C(=O)C=CC1=O)N(CC(=O)NCCCC(=O)ON1C(=O)CCC1=O)C(=O)CN1C(=O)C=CC1=O)NCCCC(=O)ON1C(=O)CCC1=O]

Compound 180 (330 mg, 0.47 mmol) was dissolved in dioxane (3 mL) and treated with 25% HCl (3 mL) at r.t. for 2 h. The reaction was concentrated and re-dissolved in DMF (5 mL) and cooled to 0° C., N-hydroxysuccinimide (113 mg, 0.98 mmol) and EDC·HCl (189 mg, 0.98 mmol) were added in sequence. The reaction was warmed to r.t. and stirred overnight, poured into ice-water, and extraction with dichloromethane (3×20 mL). The combined organic phase was washed with water (5 mL), brine (5 mL), dried over anhydrous sodium sulfate, filtered and concentrated to give a white solid 181 (369 mg, 100% yield). ESI-MS m/z 787.21 ([M+H]⁺).

### Example 213. Synthesis of (S)—N, N′-(((((2S, 21S)-11, 12-bis(2-(2, 5-dioxo-2, 5-dihydro-1H-pyrrol-1-yl)acetyl)-2, 21-dimethyl-4, 9, 14, 19-tetraoxo-3, 8, 11, 12, 15, 20-hexaazadocosane-1, 22-dioyl)bis(azanediyl))bis(4, 1-phenylene))bis(methylene))bis(1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium) (182)

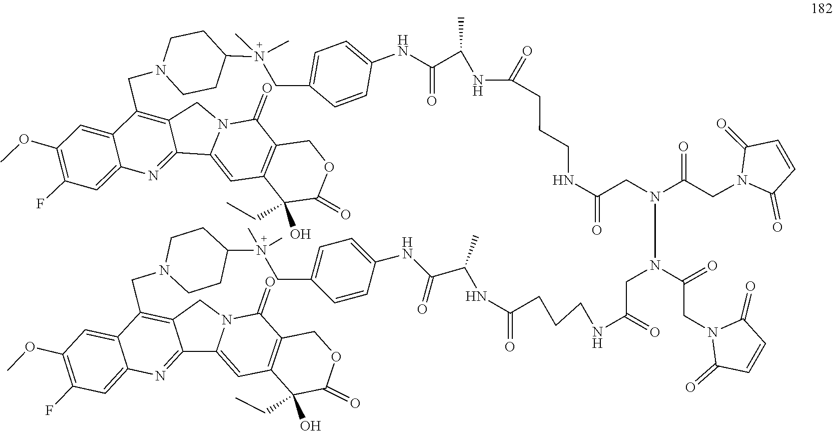

Compound 181 (31.5 mg, 0.04 mmol) was dissolved in DMA (5 mL), to which compound 24 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 182 (57 mg, 72% yield). ESI-MS m/z: M²⁺ calcd. for C₁₀₂H₁₁₆F₂N₁₈02: 991.42; found 991.86.

### Example 214. Synthesis of Methyl 4-(bis(2-(acetylthio)ethyl)amino)-4-oxobutanoate (183)

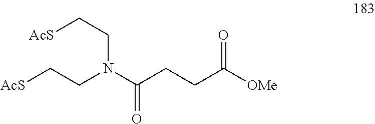

Methyl 4-(bis(2-((methylsulfonyl)oxy)ethyl)amino)-4-oxobutanoate (fresh made, 90% pure, 8.5 g, ˜20 mmol) in DMA (350 mL) at 0° C. was added thioacetic acid (10 mL, 134 mmol), followed by triethylamine (30 mL, 215 mmol). The mixture was then stirred at r. t. overnight, concentrated, diluted with EtOAc (350 mL), washed with sat'ed NaHCO₃ (300 mL), brine (300 mL) and 1 M NaH₂PO₄ (300 mL). The organic layer was dried over Na₂SO₄, filtered, evaporated and purified on silica gel column eluted with EtOAc/hexane (10%˜25% EtOAc) to afford the title compound (5.1 g, 76% yield). ESI-MS m/z [M+Na]⁺: calcd. for C₁₃H₂₁NO₅S₂ 358.1; found 358.2.

### Example 215. Synthesis of 4-(Bis(2-(pyridin-2-yldisulfanyl)ethyl)amino)-4-oxobutanoic acid (184)

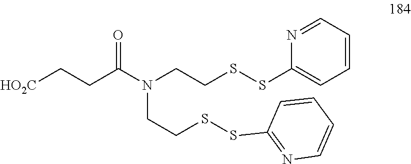 [SMILES: O=C(O)CCC(=O)N(CCSSc1ccccn1)CCSSc1ccccn1]

Methyl 4-(bis(2-(acetylthio)ethyl)amino)-4-oxobutanoate (5.0 g, 14.9 mmol) in THF (150 mL) was added NaOH (5.0 g, 125 mmol) in water (100 mL). The mixture was stirred at r.t. for 35 min, neutralized with H₃PO₄ to pH 7. Then PySSPy (26.0 g, 118 mmol) in THF (100 mL) was added and the mixture was stirred for 4 h, concentrated and purified on silica gel column, eluted with MeOH/dichloromethane/HOAc (1:20/0.2) to afford the title product (5.8 g, 85.6% yield). ESI-MS m/z [M+Na]⁺: calcd. for C₁₈H₂₁N₃O₃S₄ 478.0; found 478.2.

### Example 216. Synthesis of 2, 5-dioxopyrrolidin-1-yl 4-(bis(2-(pyridin-2-yldisulfanyl)ethyl)amino)-4-oxobutanoate (185)

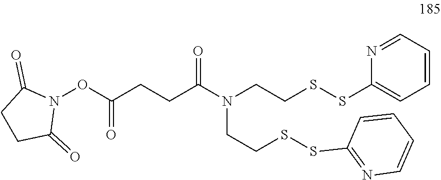 [SMILES: O=C(CCC(=O)N(CCSSc1ccccn1)CCSSc1ccccn1)ON1C(=O)CCC1=O]

To a solution of 4-(bis(2-(pyridin-2-yldisulfanyl)ethyl)amino)-4-oxobutanoic acid (5.2 g, 11.5 mmol) in DMA (100 mL) were added N-hydroxysuccinimide (1.6 g, 13.9 mmol) and EDC·HCl (5.0 g, 26.1 mmol). The mixture was stirred overnight, evaporated and purified on silica gel column, eluted with EtOAc/dichloromethane (5% to 15% EtOAc) to afford the title product (5.8 g, 85.6% yield). ESI-MS m/z [M+Na]⁺: calcd. for C₂₂H₂₄N₄O₅S₄ 575.1; found 575.2.

### Example 217. Synthesis of N-(4-((S)-2-(4-(bis(2-(pyridin-2-yldisulfanyl)ethyl) amino)-4-oxobutanamido)propanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium (186)

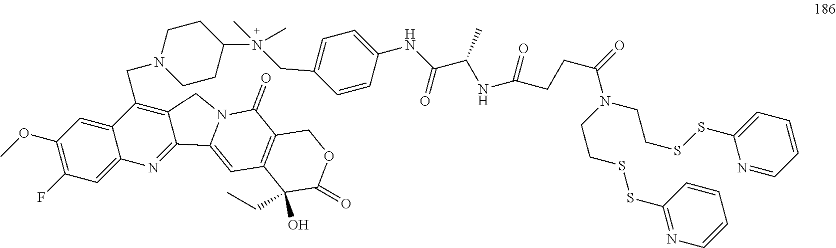

Compound 185 (23 mg, 0.04 mmol) was dissolved in DMA (5 mL), to which compound 24 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 186 (39 mg, 85% yield). ESI-MS m/z: M⁺ calcd. for C₅₇H₆₅FN₉08S₄: 1150.38; found 1150.45.

### Example 218. Synthesis of (S)-1-benzyl 5-tert-butyl 2-(14-(benzyloxy)-14-oxotetradecanamido) pentanedioate (187)

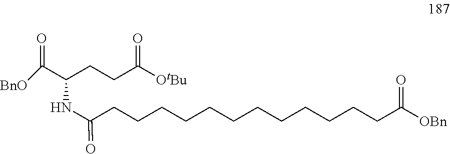 [SMILES: CC(C)(C)OC(=O)CC[C@H](NC(=O)CCCCCCCCCCCCC(=O)OCc1ccccc1)C(=O)OCc1ccccc1]

A solution of (S)-1-benzyl 5-tert-butyl 2-aminopentanedioate, HCl salt (8.70 g, 26.39 mmol), 14-(benzyloxy)-14-oxotetradecanoic acid (9.19 mmol), DIPEA (8.0 mL, 46.0 mmol) and EDC (15.3 g, 80.50 mmol) in DCM (200 mL) was stirred at room temperature for 6 hours. The mixture was diluted with water (100 mL) and separated. The aqueous phase was extracted with DCM (100 mL). The organic phases were combined, washed with brine, dried over Na₂SO₄, filtered, concentrated and purified on a silica gel column (dichloromethane/EtOAc=20:1 to 5:1) to give the title compound (13.65 g, 83% yield). MS-ESI m/z: [M+H]⁺ calcd. for C₃₇H₅₄NO₇, 624.38; found, 624.38.

### Example 219. Synthesis of (S)-5-(benzyloxy)-4-(14-(benzyloxy)-14-oxotetradecanamido)-5-oxopentanoic acid (188)

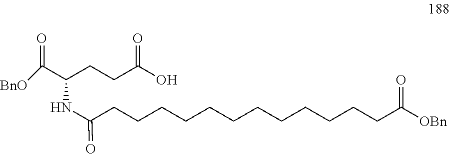 [SMILES: O=C(O)CC[C@H](NC(=O)CCCCCCCCCCCCC(=O)OCc1ccccc1)C(=O)OCc1ccccc1]

Compound 187 (12.50 g, 20.05 mmol) was dissolved in dioxane (30 mL) at 4° C., and treated with hydrochloric acid (10 mL, 36% conc) for 0.5 hours. The reaction mixture was diluted with toluene (20 mL) and DMF (20 mL), evaporated at 15° C. to give the title compound 188 (11.26 g, 99% yield). MS-ESI m/z: [M+H]⁺ calcd. for C₃₃H₄₆NO₇, 568.32; found, 568.34.

### Example 220. Synthesis of (S)-35, 49-dibenzyl 1-tert-butyl 16, 32, 37-trioxo-3, 6, 9, 12, 19, 22, 25, 28-octaoxa-15, 31, 36-triazanonatetracontane-1, 35, 49-tricarboxylate (189)

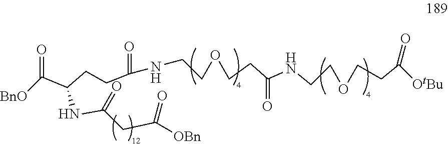

A mixture of compound 188 (10.70 g, 18.86 mmol), tert-butyl 1-amino-15-oxo-3, 6, 9, 12, 19, 22, 25, 28-octaoxa-16-azahentriacontan-31-oate HCl salt (11.45 g, 18.93 mmol), EDC (9.51 g, 50.01 mmol) and DIPEA (4.00 mL, 23.00 mol) in DCM (200 mL) was stirred overnight, diluted with brine (100 mL) and separated. The aqueous phase was extracted with DCM (100 mL). The organic phases were combined, washed with brine, dried over Na₂SO₄, filtered, concentrated and purified on a silica gel column (dichloromethane/EtOAc=10:1 to 4:1) to give the title compound 189 (18.15 g, 86% yield). MS-ESI m/z: [M+H]⁺ calcd. for C₅₉H₉₆N₃O₁₇, 1118.67; found, 1118.80.

### Example 221. Synthesis of (S)-18-((benzyloxy)carbonyl)-3, 16, 21, 37-tetraoxo-1-phenyl-2, 25, 28, 31, 34, 41, 44, 47, 50-nonaoxa-17, 22, 38-triazatripentacontan-53-oic acid (190)

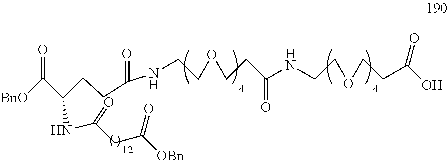

Compound 189 (10.50 g, 9.39 mmol) was dissolved in dioxane (45 mL) at 4° C., and treated with hydrochloric acid (15 mL, 36% conc) for 0.5 hours. The reaction mixture was diluted with toluene (20 mL) and DMF (20 mL), evaporated at 15° C. and purified on a silica gel column (dichloromethane/MeOH=10:1 to 6:1) to give the title compound 190 (8.67 g, 87% yield). MS-ESI m/z: [M+H]⁺ calcd. for C₅₅H₈₈N₃O₁₇, 1062.60; found, 1062.68.

### Example 222. Synthesis of (18S, 59S)-18-((benzyloxy)carbonyl)-59-((tert-butoxycarbonyl)amino)-3, 16, 21, 37, 53-pentaoxo-1-phenyl-2, 25, 28, 31, 34, 41, 44, 47, 50-nonaoxa-17, 22, 38, 54-tetraazahexacontan-60-oic acid (191)

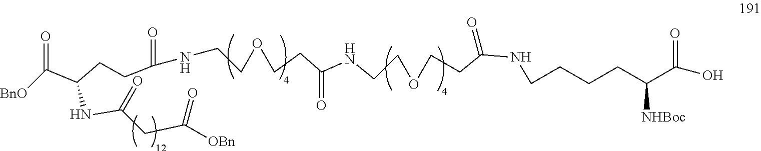

A solution of compound 190 (8.50 g, 8.01 mmol), N-hydroxysuccinimide (3.20 g, 27.82 mmol), EDC (10.28 g, 54.10 mmol) and DIPEA (6.00 mL, 34.51 mmol) in THF (150 mL) was stirred for 6 h and evaporated in vacuo to give a NHS ester crude product.

To a solution of (S)-6-amino-2-((tert-butoxycarbonyl)amino)hexanoic acid, HCl salt (2.75 g, 9.73 mmol) in DMF (100 mL) and 1.0 M Na₂PO₄ (pH 7.5, 55 mL), the above prepared ester was added in four portion in 1 h. The reaction mixture was stirred at room temperature for another 3 hours. After concentration, the residue was purified on a silica gel column (dichloromethane/MeOH=10:1 to 4:1) to give the title compound (8.16 g, 79% yield). MS-ESI m/z: [M+H]⁺ calcd. for C₆₆H₁₀₈N₅O₂₀, 1289.75; found, 1289.90.

### Example 223. Synthesis of (18S, 59S)-59-amino-18-((benzyloxy)carbonyl)-3, 16, 21, 37, 53-pentaoxo-1-phenyl-2, 25, 28, 31, 34, 41, 44, 47, 50-nonaoxa-17, 22, 38, 54-tetraazahexacontan-60-oic acid, HCl salt (192)

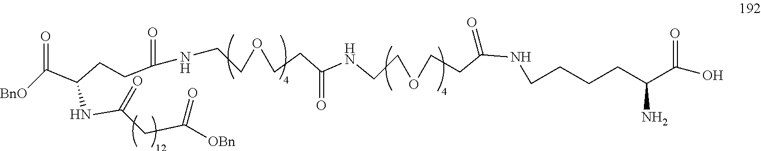

Compound 191 (8.10 g, 6.28 mmol) was dissolved in dioxane (40 mL) at 4° C., and treated with hydrochloric acid (15 mL, 36% conc) for 0.5 hours. The reaction mixture was diluted with toluene (20 mL) and DMF (20 mL), evaporated at 15° C. to give the crude title compound 192 (7.71 g, 100% yield) for next step without further purification. MS-ESI m/z: [M+H]⁺ calcd. for C₆₁H₈₈N₃O₁₇, 1190.70; found, 1190.78.

### Example 224. Synthesis of (S)-2-(4-(2, 5-dioxo-2, 5-dihydro-1H-pyrrol-1-yl)butanamido)-propanoic acid (193)

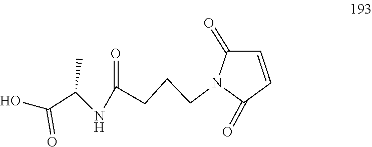 [SMILES: C[C@H](NC(=O)CCCN1C(=O)C=CC1=O)C(=O)O]

To a solution of N-succinimidyl 4-maleimido-butyrate (7.10 g, 25.35 mmol) and alanine (3.01 g, 33.80 mmol) in DMF (50 mL) at 0° C., DIPEA (10 mL) was added. The reaction mixture was stirred at 0° C. for 0.5 h, followed by at room temperature for 1 h. Then the reaction mixture was concentrated and purified on silica gel column (mobile phase: DCM/MeOH=10:1 with 0.1% formic acid) to afford compound 193 (5.21 g, 81% yield). MS-ESI m/z: [M+H]⁺ calcd. for C₁₁H₁₄N₂O₅, 255.09; found, 255.15.

### Example 225. Synthesis of (S)-2, 5-dioxopyrrolidin-1-yl 2-(4-(2, 5-dioxo-2, 5-dihydro-1H-pyrrol-1-yl)butanamido)propanoate (194)

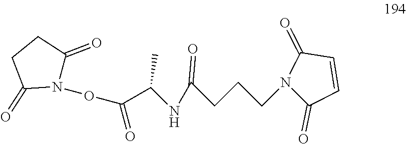 [SMILES: C[C@H](NC(=O)CCCN1C(=O)C=CC1=O)C(=O)ON1C(=O)CCC1=O]

A solution of compound 193 (5.15 g, 20.26 mmol), N-hydroxysuccinimide (2.80 g, 24.34 mmol), EDC (10.28 g, 54.10 mmol) and DIPEA (5.50 mL, 31.63 mmol) in DCM (70 mL) was stirred for 6 h, evaporated in vacuo and purified on silica gel column (mobile phase: DCM/EtOAc=10:1) to afford compound 194 (5.83 g, 82% yield). MS-ESI m/z: [M+H]⁺ calcd. for C₁₅H₁₇N₃O₇, 351.11; found, 351.20.

### Example 226. Synthesis of (18S, 59S)-18-((benzyloxy)carbonyl)-59-((S)-2-(4-(2, 5-dioxo-2, 5-dihydro-1H-pyrrol-1-yl)butanamido)propanamido)-3, 16, 21, 37, 53-pentaoxo-1-phenyl-2, 25, 28, 31, 34, 41, 44, 47, 50-nonaoxa-17, 22, 38, 54-tetraazahexacontan-60-oic acid (195)

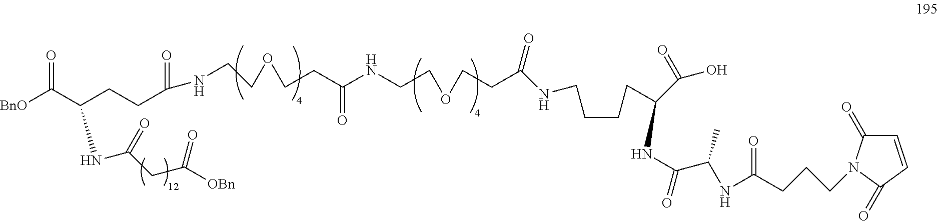

To a solution of compound 192 7.61 g, 6.39 mmol) an compound 194 2.90 g, 8.280 mmol) in DMF (40 mL) at 0° C., DIPEA (7 mL) was added. The reaction mixture was stirred at 0° C. for 0.5 h, followed by at room temperature for 1 h. Then the reaction mixture was concentrated and purified on silica gel column (mobile phase: DCM/MeOH=10:1 with 0.1% formic acid) to afford compound 195 (7.10 g, 78% yield). MS-ESI m/z: [M+H]⁺ calcd. for C₇₂H₁₁₂N₇O₂₂, 1426.7782; found, 1426.7820.

### Example 227. Synthesis of (18S, 59S)-18-((benzyloxy)carbonyl)-59-((S)-2-(4-(2, 5-dioxo-2, 5-dihydro-1H-pyrrol-1-yl)butanamido)propanamido)-3, 16, 21, 37, 53, 60, 63, 66, 69-nonaoxo-1-phenyl-2, 25, 28, 31, 34, 41, 44, 47, 50-nonaoxa-17, 22, 38, 54, 61, 64, 67, 70-octaazadoheptacontan-72-oic acid (196)

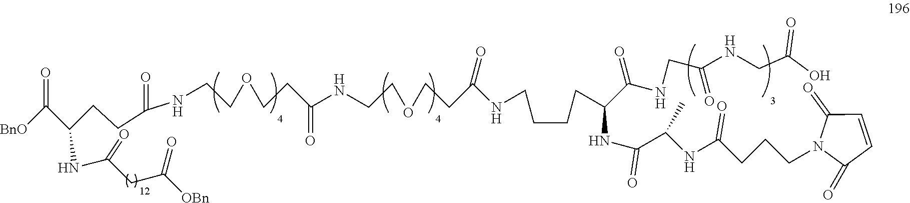

A solution of compound 195 (7.05 g, 4.94 mmol), N-hydroxysuccinimide (0.92 g, 8.00 mmol), EDC (3.01 g, 15.84 mmol) and DIPEA (1.00 mL, 5.75 mmol) in THF (50 mL) was stirred for 6 h and evaporated in vacuo to give a crude NHS ester.

To a solution of 2-(2-(2-aminoacetamido)acetamido)acetic acid (Gly-Gly-Gly) HCl salt (1.67 g, 7.40 mmol) in DMF (40 mL) and 1.0 M Na₂PO₄ (pH 7.5, 15 mL), the above ester was added in four portions in 1 h. The reaction mixture was stirred at room temperature for another 3 hours. After concentration, the residue was purified on a silica gel column (dichloromethane/MeOH=10:1 to 7:1) to give the title compound 196 (8.16 g, 79% yield). MS-ESI m/z: [M+H]⁺ calcd. for C₇₈H₁₂₁N₁₀O₂₅, 1597.8426; found, 1597.8495.

### Example 228. Synthesis of N-(4-((18S, 61S, 76S)-18-((benzyloxy)carbonyl)-61-((S)-2-(4-(2, 5-dioxo-2, 5-dihydro-1H-pyrrol-1-yl)butanamido)propanamido)-76-methyl-3, 16, 21, 38, 55, 62, 65, 68, 71, 74-decaoxo-1-phenyl-2, 25, 29, 32, 35, 42, 46, 49, 52-nonaoxa-17, 22, 39, 56, 63, 66, 69, 72, 75-nonaazaheptaheptacontanamido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium formate (197)

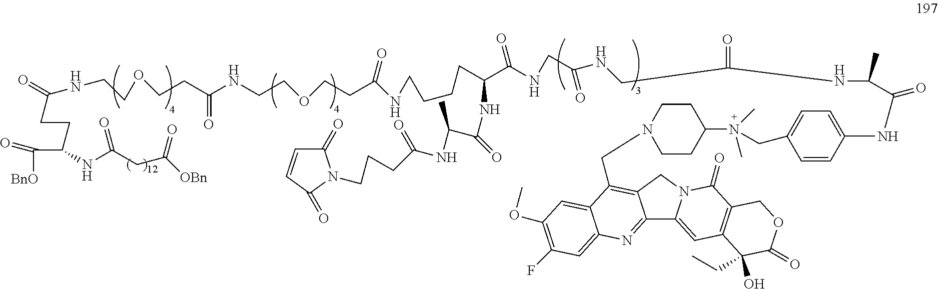

A solution of compound 196 (251 mg, 0.157 mmol), compound 24 (147.8 mg, 0.157 mmol), EDC (101 mg, 0.526 mmol) and DIPEA (0.10 mL, 0.575 mmol) in DMA (10 mL), was stirred at room temperature for 6 h. The mixture was evaporated in vacuo and purified by preparative C-18 HPLC (acetonitrile/water containing 0.5% formic acid, Φ=3 cm, v=20 mL/min, 90% water to 30% water in 45 min) to give compound 197 (235.8 mg, 62% yield). ESI-MS m/z: M⁺ calcd. for C₁₂₁H₁₇₁FN₁₇O₃₁: 2377.2305; found 2377.2415.

### Example 229. Synthesis of N-(4-((2S, 17S, 60S)-60, 74-dicarboxy-17-((S)-2-(4-(2, 5-dioxo-2, 5-dihydro-1H-pyrrol-1-yl)butanamido)propanamido)-2-methyl-4, 7, 10, 13, 16, 23, 40, 57, 62-nonaoxo-26, 29, 32, 36, 43, 46, 49, 53-octaoxa-3, 6, 9, 12, 15, 22, 39, 56, 61-nonaazatetraheptacontan-amido)benzyl)-1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-N, N-dimethylpiperidin-4-aminium (198)

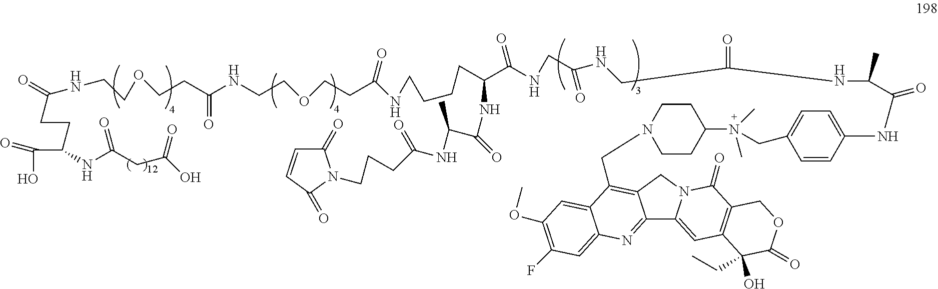

Compound 197 (110 mg, 0.0454 mmol) in DCM (2 mL) was treated with TFA (4 mL) for 1 hour. The reaction mixture was diluted with toluene (5 mL) and DMF (5 mL), evaporated, and by preparative C-18 HPLC (acetonitrile/water containing 0.5% formic acid, Φ=3 cm, v=20 mL/min, 95% water to 30% water in 45 min) to give compound 198 (70.2 mg, 69% yield). ESI-MS m/z: M⁺ calcd. for C₁₀₇H₁₅₉FN₁₇O₃₁: 2197.1366; found 2197.1410.

### Example 230. Synthesis of (S)-tert-butyl (2-((2-((2-((1-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)amino)-2-oxoethyl)amino)-2-oxoethyl)amino)-2-oxoethyl)carbamate (199)

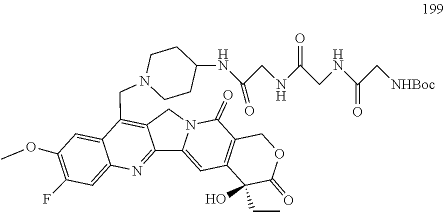 [SMILES: CC[C@@]1(O)C(=O)OCc2c1cc1n(c2=O)Cc2c-1nc1cc(F)c(OC)cc1c2CN1CCC(NC(=O)CNC(=O)CNC(=O)CNC(=O)OC(C)(C)C)CC1]

In a solution of (S)-11-((4-aminopiperidin-1-yl)methyl)-4-ethyl-8-fluoro-4-hydroxy-9-methoxy-1H-pyrano[3′, 4′:6, 7]indolizino[1, 2-b]quinoline-3, 14(4H, 12H)-dione, HCl salt (49) (0.805 g, 1.478 mmol) in DMF (25 mL) and 0.1 M NaH₂PO₄ pH 7.5 (50 mL), 2, 5-dioxopyrrolidin-1-yl 2, 2-dimethyl-4, 7, 10-trioxo-3-oxa-5, 8, 11-triazatridecan-13-oate (0.855 g, 2.214 mmol) was added in 4 portions in 3 h. After addition, the mixture was stirred for another 2 h, concentrated, extracted with EtOAc/n-butanol (1:1, 15 mL×3). The organic layers were combined, concentrated and purified on a silica gel column (dichloromethane/MeOH=12:1 to 7:1) to give the title compound 199 (0.841 g, 73% yield). MS-ESI m/z: [M+H]⁺ calcd. for C₃₈H₄₇FN₇O₁₀, 780.3369; found, 780.3415.

### Example 231. Synthesis of (S)-2-amino-N-(2-((2-((1-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)amino)-2-oxoethyl)amino)-2-oxoethyl)acetamide, HCl salt (200)

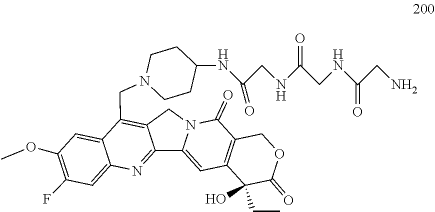 [SMILES: CC[C@@]1(O)C(=O)OCc2c1cc1n(c2=O)Cc2c-1nc1cc(F)c(OC)cc1c2CN1CCC(NC(=O)CNC(=O)CNC(=O)CN)CC1]

Compound 199 (0.810 g, 1.039 mmol) was dissolved in dioxane (25 mL) at 4° C., and treated with hydrochloric acid (10 mL, 36% conc) for 0.5 hours. The reaction mixture was diluted with toluene (15 mL) and DMF (15 mL), evaporated at 15° C. to give the crude title compound 200 (0.744 g, 100% yield) for next step without further purification. MS-ESI m/z: [M+H]⁺ calcd. for C₃₃H₃₉FN₇08, 680.2845; found, 680.2895.

### Example 232. Synthesis of (2S, 10S, 11S, 19S)-2, 19-bis((S)-18-((benzyloxy)carbonyl)-3, 16, 21, 37, 53-pentaoxo-1-phenyl-2, 25, 28, 31, 34, 41, 44, 47, 50-nonaoxa-17, 22, 38, 54-tetraazaoctapentacontan-58-yl)-10, 11-bis(4-(2, 5-dioxo-2, 5-dihydro-1H-pyrrol-1-yl)butanamido)-4, 9, 12, 17-tetraoxo-3, 8, 13, 18-tetraazaicosane-1, 20-dioic acid (201)

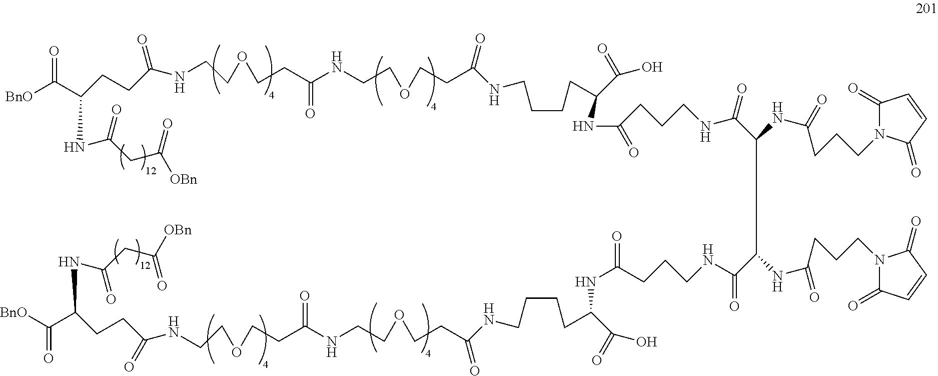

To a solution of compound 192 (2.78 g, 2.267 mmol) and compound 132 (0.951 g, 1.129 mmol) in DMF (40 mL) at 0° C., DIPEA (6 mL) was added. The reaction mixture was stirred at 0° C. for 0.5 h, followed by at room temperature for 1 h. Then the reaction mixture was concentrated and purified on silica gel column (mobile phase: DCM/MeOH=10:1 to 3:1 with 0.1% formic acid) to afford compound 201 (2.432 g, 72% yield). MS-ESI m/z: [M+H]⁺ calcd. for C₁₅₀H₂₃₁N₁₆O₄₆, 2992.6229; found, 2992.6295.

### Example 233. Synthesis of (15S, 56S, 64S, 65S, 73S, 114S)-tetrabenzyl 64, 65-bis(4-(2, 5-dioxo-2, 5-dihydro-1H-pyrrol-1-yl)butanamido)-56, 73-bis((2-((2-((2-((1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-piperidin-4-yl)amino)-2-oxoethyl)amino)-2-oxoethyl)amino)-2-oxoethyl)carbamoyl)-13, 18, 34, 50, 58, 63, 66, 71, 79, 95, 111, 116-dodecaoxo-22, 25, 28, 31, 38, 41, 44, 47, 82, 85, 88, 91, 98, 101, 104, 107-hexadecaoxa-14, 19, 35, 51, 57, 62, 67, 72, 78, 94, 110, 115-dodecaazaoctacosahectane-1, 15, 114, 128-tetracarboxylate (202)

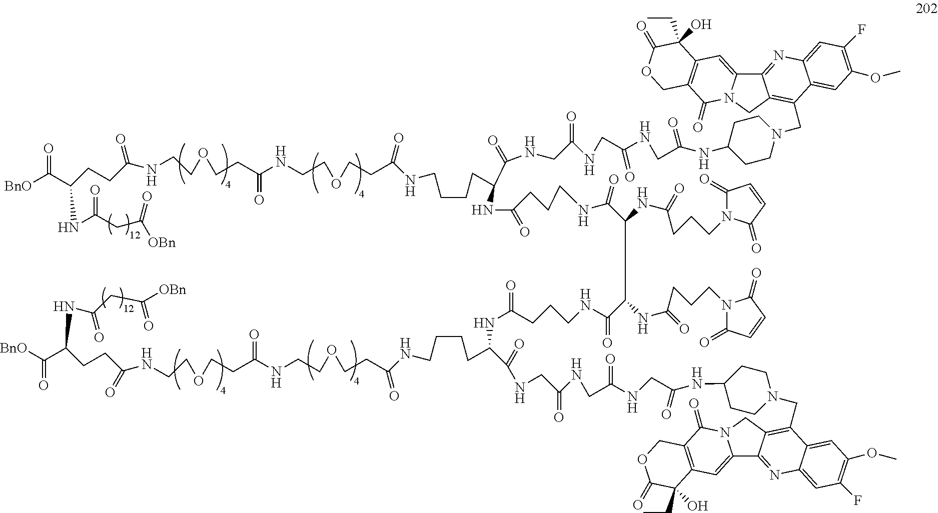

A solution of compound 201 (0.150 g, 0.209 mmol), compound 200 (0.312 g, 0.104 mmol), EDC (0.252 g, 1.311 mmol) in DMF (8 mL) was stirred for 8 h, evaporated in vacuo and purified on a silica gel column (dichloromethane/MeOH=10:1 to 7:1) to give the title compound 202 (0.301 g, 67% yield). MS-ESI m/z: [M+H]⁺ calcd. for C₂₁₆H₃₀₃F₂N₃₀06₀, 4315.1550; found, 4315.1685.

### Example 234. Synthesis of (15S, 56S, 64S, 65S, 73S, 114S)-64, 65-bis(4-(2, 5-dioxo-2, 5-dihydro-1H-pyrrol-1-yl)butanamido)-56, 73-bis((2-((2-((2-((1-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)piperidin-4-yl)amino)-2-oxoethyl)amino)-2-oxoethyl)amino)-2-oxoethyl)carbamoyl)-13, 18, 34, 50, 58, 63, 66, 71, 79, 95, 111, 116-dodecaoxo-22, 25, 28, 31, 38, 41, 44, 47, 82, 85, 88, 91, 98, 101, 104, 107-hexadecaoxa-14, 19, 35, 51, 57, 62, 67, 72, 78, 94, 110, 115-dodecaazaoctacosahectane-1, 15, 114, 128-tetracarboxylic acid (203)

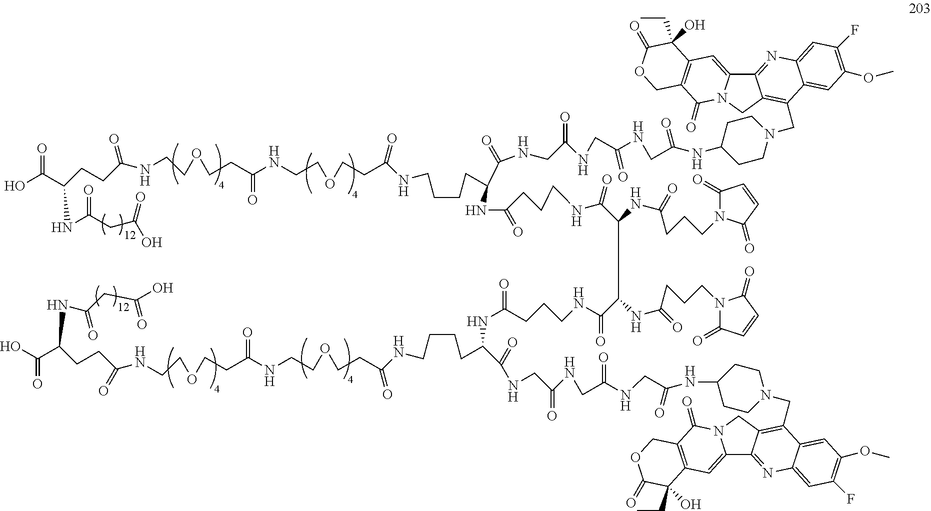

Compound 202 (105 mg, 0.0243 mmol) in DCM (2 mL) was treated with TFA (4 mL) for 1 hour. The reaction mixture was diluted with toluene (5 mL) and DMF (5 mL), evaporated, and purified by preparative C-18 HPLC (acetonitrile/water containing 0.5% formic acid, Φ=3 cm, v=20 mL/min, 95% water to 30% water in 45 min) to give compound 203 (65.3 mg, 68% yield). ESI-MS m/z: [M+H]⁺ calcd. for C₁₈₈H₂₇₉F₂N₃₀O₆₀: 3954.9672; found 3954.9785.

### Example 235. Synthesis of (115, 19S, 20S, 28S)-di-tert-butyl 19, 20-bis(((benzyloxy)-carbonyl)amino)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioate (204)

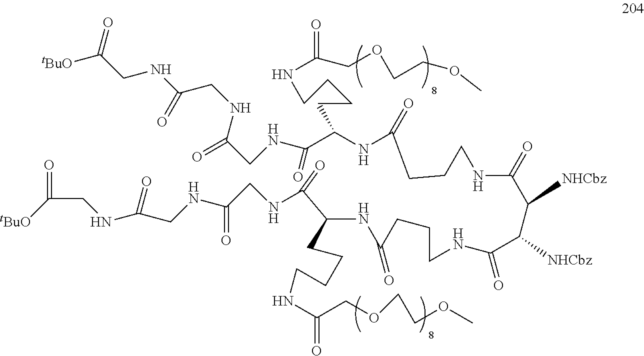

To the solution of (S)-tert-butyl 34-(4-aminobutanamido)-28, 35, 38, 41-tetraoxo-2, 5, 8, 11, 14, 17, 20, 23, 26-nonaoxa-29, 36, 39, 42-tetraazatetratetracontan-44-oate (4.427 g, 5.01 mmol) in DMF (80 mL) were added DIPEA (2.0 mL, 11.503 mmol) and compound 127 (2.001 g, 5.02 mmol). The mixture was stirred at r.t. overnight, followed by addition of EDC (3.851 g, 20.05 mmol). The mixture was continued stirring for 8 h, concentrated under reduced pressure and purified by silica gel column chromatography with a gradient of 5-20% methanol in DCM to deliver the title product (8.491 g, 79% yield). MS ESI m/z calcd. for C₉₈H₁₆₅N₁₄O₃₈ [M+H]⁺ 2146.1410, found 2146.1985.

### Example 236. Synthesis of (11S, 19S, 20S, 28S)-di-tert-butyl 19, 20-diamino-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaocta triacontane-1, 38-dioate (205)

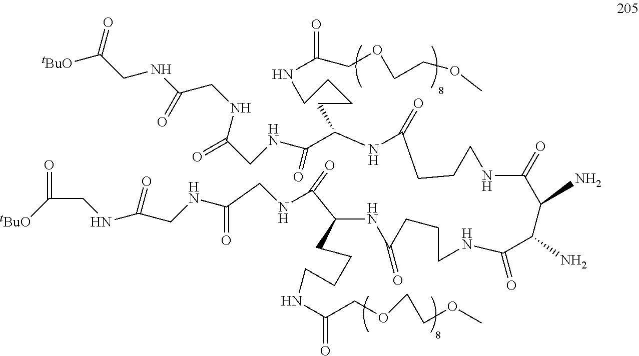

To a solution of compound 204 (8.450 g, 3.939 mmol) in DMA (100 mL) was added Pd/C (1.00 g, 10 wt %, 50% wet) in a hydrogenation bottle. The mixture was shaken with 40 psi of H₂ overnight, filtered through Celite (filter aid), and the filtrate was concentrated to afford light brown colored gum (7.2458 g, 98% yield) which was used for the next step without further purification. MS ESI m/z calcd. for C₈₂H₁₅₃N₁₄O₃₄ [M+H]²⁺ 939.5377, found 939.5485.

### Example 237. Synthesis of (11S, 19R, 20S, 28S)-di-tert-butyl 19, 20-bis(2, 5-dioxo-2, 5-dihydro-1H-pyrrol-1-yl)-4, 7, 10, 13, 18, 21, 26, 29, 32, 35-decaoxo-I1, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioate (206)

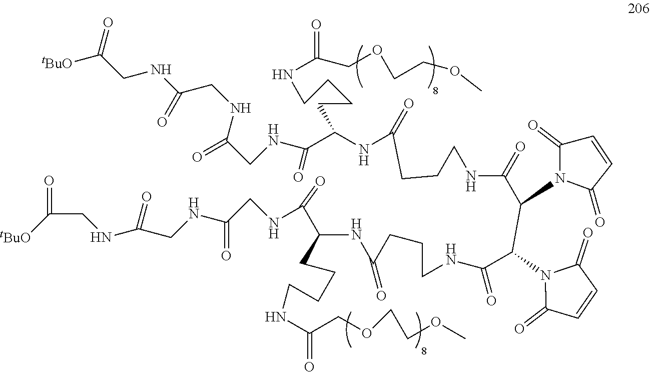

To a mixture of compound 205 (7.201 g, 3.836 mmol) in saturated solution of NaHCO₃ (90 mL) and MeOH (10 mL) cooled at 0° C., N-(methoxycarbonyl)maleimide (3.10 g, 20.00 mmol) was added to the stirred solution. After 20 mins the reaction mixture was diluted with water (150 mL) and stirred for 30 min at room temperature. The reaction mixture was concentrated at 2-8° C. to ˜100 mL and extracted with DCM (4×60 mL). The organic layers were combined, dried over MgSO₄, filtered, concentrated and purified by silica gel column chromatography with a gradient of 5-20% methanol in DCM to give the title product (8.491 g, 79% yield). MS ESI m/z calcd. for C₉₀H₁₅₃N₁₄O₃₈ [M+H]⁺ 2038.0471, found 2038.0545.

### Example 238. Synthesis of (11S, 19R, 205, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioic acid (207)

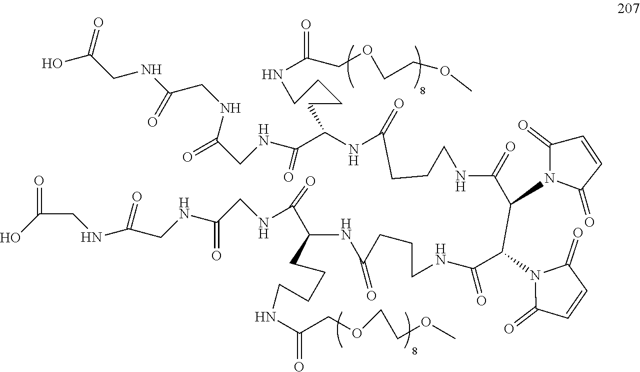

To a solution of compound 206 (8.451 g, 4.148 mmol) in dioxane (50 mL) on ice bath was added HCl (conc. 12 mL). The mixture was stirred on the ice bath for 45 min, diluted with dioxane (50 mL) and toluene (50 mL), concentrated, and co-evaporated with dioxane/toluene (1:1, 2×50 mL) in vacuum to afford colorless gum (7.745 g, 97% yield) which was 93% pure by HPLC and can be used for the next step without further purification. The crude compound can be purified by silica gel column chromatography with a gradient of 3-10% water in acetone to give the title product (7.141 g, 84% yield). The MS ESI m/z calcd. for C₈₂H₁₃₇N₁₄O₃₈ [M+H]⁺ 1925.9219, found 1925.9395.

### Example 239. Synthesis of (2R, 3S)-2, 3-bis(2, 5-dioxo-2, 5-dihydro-1H-pyrrol-1-yl)-N₁, N₄-bis((S)-34-((2-((2-((2-(((1S, 9S)-9-ethyl-5-fluoro-9-hydroxy-4-methyl-10, 13-dioxo-1, 2, 3, 9, 10, 12, 13, 15-octahydrobenzo[de]pyrano[3′, 4′:6, 7]indolizino[1, 2-b]quinolin-1-yl)amino)-2-oxoethyl)amino)-2-oxoethyl)amino)-2-oxoethyl)carbamoyl)-28, 36-dioxo-2, 5, 8, 11, 14, 17, 20, 23, 26-nonaoxa-29, 35-diazanonatriacontan-39-yl)succinamide (208)

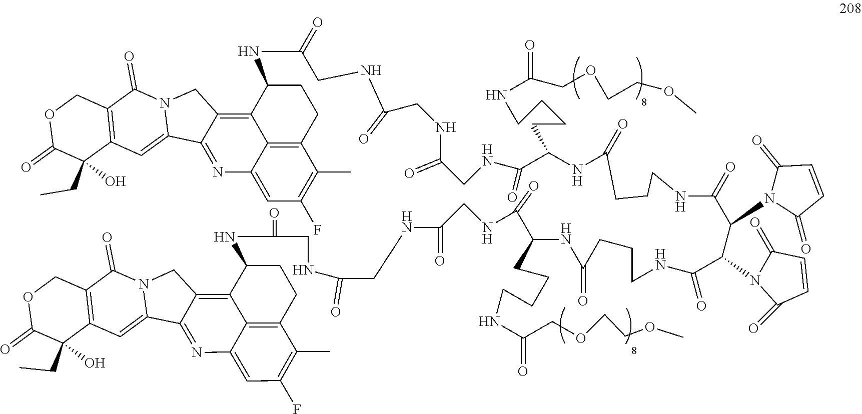

To a solution of compound 207 (0.301 g, 0.156 mmol), exatecan HCl salt (0.151 g, 0.318 mmol), EDC (0.150 g, 0.781 mmol) in DMA (8 mL), DIPEA (0.080 mL, 0.460 mmol) was added. Then the mixture was stirred at r.t. for 6 h, concentrated under reduced pressure and purified by silica gel column chromatography with a gradient of 3-18% methanol in DCM to give the title product (0.207 g, 72% yield). MS ESI m/z calcd. for C₁₃₀H₁₇₆F₂N₂₀04₄ [M+H]⁺ 2760.2196, found 2760.2450.

### Example 240. Synthesis of (2R, 3S)-2, 3-bis(2, 5-dioxo-2, 5-dihydro-1H-pyrrol-1-yl)-N¹, N⁴-bis((S)-34-((2-((2-((2-((4-(hydroxymethyl)phenyl)amino)-2-oxoethyl)amino)-2-oxoethyl)amino)-2-oxoethyl)carbamoyl)-28, 36-dioxo-2, 5, 8, 11, 14, 17, 20, 23, 26-nonaoxa-29, 35-diazanonatriacontan-39-yl)succinamide (209)

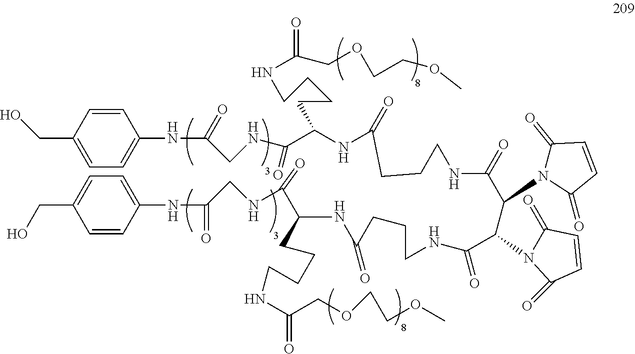

To a solution of compound 207 (1.008 g, 0.523 mmol) and (4-aminophenyl)methanol HCl salt (0.261 g, 1.635 mmol) in DMA (15 mL) were added EDC (0401 g, 2.088 mmol) and DIPEA (0.20 mL, 1.15 mmol). The mixture was stirred for 8 h, concentrated under reduced pressure and purified by silica gel column chromatography with a gradient of 5-15% methanol in DCM to give the title product (0.904 g, 81% yield). MS ESI m/z calcd. for C₉₆H₁₅₀N₁₆O₃₈ [M+H]⁺ 2136.0376, found 2136.0520.

### Example 241. Synthesis of di-tert-butyl (((((11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioyl)bis(azanediyl))bis(4, 1-phenylene))bis(methylene))-dicarbamate (210)

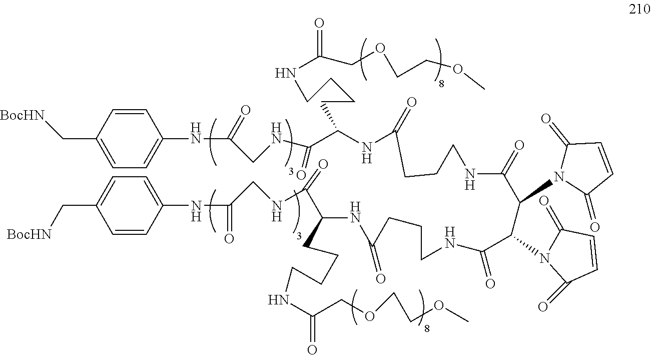

To a solution of compound 207 (1.001 g, 0.520 mmol) and tert-butyl 4-aminobenzylcarbamate, HCl salt (0.301 g, 1.163 mmol) in DMA (20 mL) were added EDC (0.401 g, 2.088 mmol) and DIPEA (0.20 mL, 1.15 mmol). The mixture was stirred for 8 h, concentrated under reduced pressure and purified by silica gel column chromatography with a gradient of 5-15% methanol in DCM to give the title product (1.007 g, 83% yield). MS ESI m/z calcd. for C₁₀₆H₁₆₉N₁₈O₄₀ [M+H]⁺ 2334.1744, found 2334.1980.

### Example 242. Synthesis of di-tert-butyl (((((11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioyl)bis(azanediyl))bis(4, 1-phenylene))bis(methylene))bis-(methylcarbamate) (211)

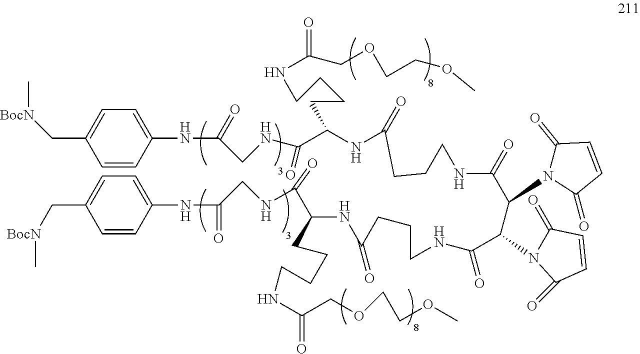

To a solution of compound 207 (1.001 g, 0.520 mmol) and tert-butyl 4-aminobenzyl(methyl)carbamate, HCl salt (0.300 g, 1.100 mmol) in DMA (20 mL) were added EDC (0.401 g, 2.088 mmol) and DIPEA (0.20 mL, 1.15 mmol). The mixture was stirred for 8 h, concentrated under reduced pressure and purified by silica gel column chromatography with a gradient of 5-15% methanol in DCM to give the title product (0.988 g, 81% yield). MS ESI m/z calcd. for C₁₀₈H₁₇₃N₁₈O₄₀ [M+H]2362.2056, found 2362.2230.

### Example 243. Synthesis of (2R, 3S)—N′, N⁴-bis((S)-34-((2-((2-((2-((4-(aminomethyl)phenyl)-amino)-2-oxoethyl)amino)-2-oxoethyl)amino)-2-oxoethyl)carbamoyl)-28, 36-dioxo-2, 5, 8, 11, 14, 17, 20, 23, 26-nonaoxa-29, 35-diazanonatriacontan-39-yl)-2, 3-bis(2, 5-dioxo-2, 5-dihydro-1H-pyrrol-1-yl)succinamide (212)

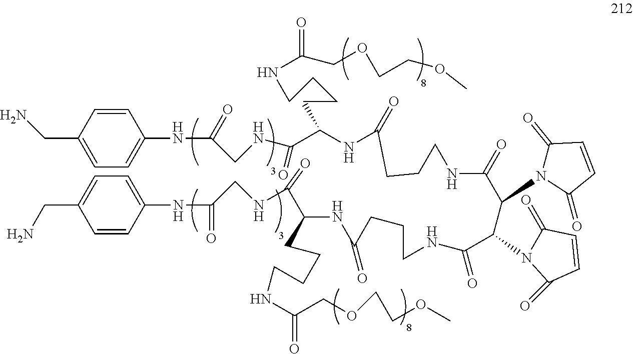

To a solution of compound 210 (0.2511 g, 0.107 mmol) in dioxane (10 mL) on an ice bath was added HCl (conc. 2 mL). The mixture was stirred on the ice bath for 30 min, diluted with dioxane (10 mL) and toluene (10 mL), concentrated and co-evaporated with dioxane/toluene (1:1, 2×10 mL) in vacuum to afford 212 HCl salt (0.2373 g, 100% yield) which was 95% pure by HPLC and used for the next step without further purification. The MS ESI m/z calcd. for C₉₆H₁₅₄N₁₈O₃₆ [M+2H]2+1067.5388, found 1067.5445.

### Example 244. Synthesis of (2R, 3S)—N¹, N⁴-bis((S)-34-((2-((2-((2-((4-(methylaminomethyl)phenyl)-amino)-2-oxoethyl)amino)-2-oxoethyl)amino)-2-oxoethyl)carbamoyl)-28, 36-dioxo-2, 5, 8, 11, 14, 17, 20, 23, 26-nonaoxa-29, 35-diazanonatriacontan-39-yl)-2, 3-bis(2, 5-dioxo-2, 5-dihydro-1H-pyrrol-1-yl)succinamide (213)

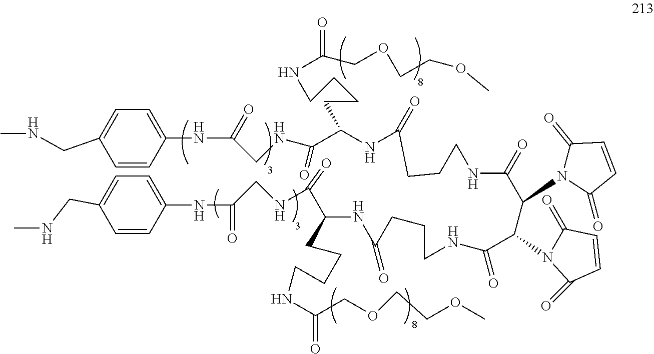

To a solution of compound 211 (0.2501 g, 0.106 mmol) in dioxane (10 mL) on an ice bath was added HCl (conc. 2 mL). The mixture was stirred on the ice bath for 30 min, diluted with dioxane (10 mL) and toluene (10 mL), concentrated and co-evaporated with dioxane/toluene (1:1, 2×10 mL) in vacuum to afford 213 HCl salt (0.2292 g, 100% yield) which was 95% pure by HPLC and used for the next step without further purification. The MS ESI m/z calcd. for C₉₈H₁₅₈N₁₈O₃₆ [M+2H]2+1082.5622, found 1082.5815.

### Example 245. Synthesis of bis((S)-4-ethyl-8-fluoro-4-hydroxy-3, 14-dioxo-3, 4, 12, 14-tetrahydro-1H-pyrano[3′, 4′:6, 7]indolizino[1, 2-b]quinolin-9-yl) (((((11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioyl)bis(azanediyl))bis(4, 1-phenylene))bis(methylene))-dicarbamate (214)

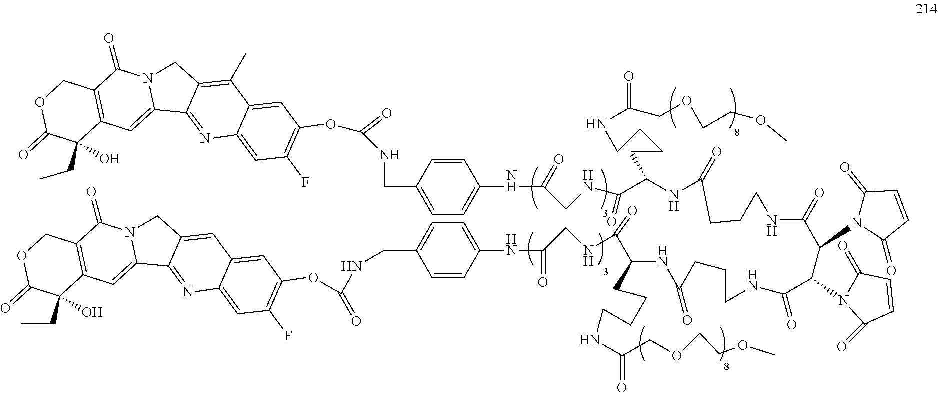

To a solution of (S)-4-ethyl-8-fluoro-4, 9-dihydroxy-1H-pyrano[3′, 4′:6, 7]indolizino[l, 2-b]quinoline-3, 14(4H, 12H)-dione (0.101 g, 0.264 mmol) in DCM (10 mL) on ice bath, DIPEA (0.050 mL, 0.287 mmol) and 4-nitrophenyl carbonochloridate (0.056 g, 0.279 mmol) were added. The mixture was then stirred at r. t. for 2 h, followed by addition of compound 212 (0.288 g, 0.135 mmol) and DIPEA (0.060 mL, 0.345 mmol). The reaction mixture was continued stirring for overnight, concentrated under reduced pressure and purified by silica gel column chromatography with a gradient of 5-15% methanol in DCM to give the title product (0.303 g, 76% yield). MS ESI m/z calcd. for C₁₃₈H₁₇₈F₂N₂₂O₄₈ [M+H]⁺ 2590.2211, found 2950.2390.

### Example 246. Synthesis of bis((S)-4-ethyl-8-fluoro-4-hydroxy-3, 14-dioxo-3, 4, 12, 14-tetrahydro-1H-pyrano[3′, 4′:6, 7]indolizino[1, 2-b]quinolin-9-yl) (((((11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioyl)bis(azanediyl))bis(4, 1-phenylene))bis(methylene))-bis(methylcarbamate) (215)

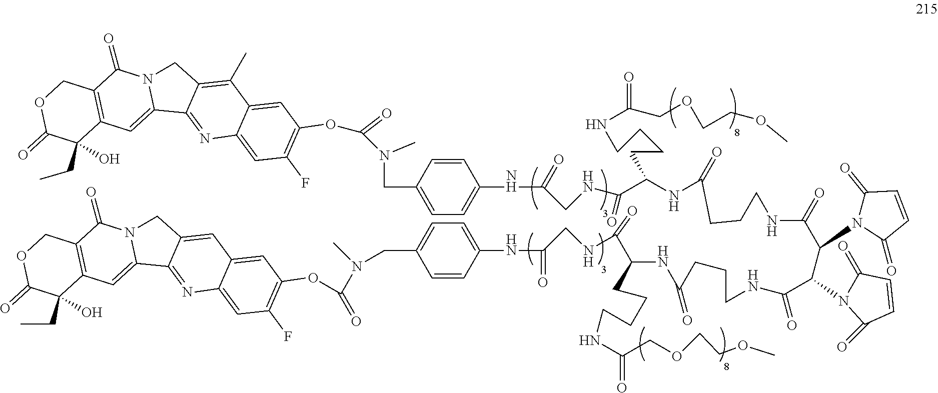

To a solution of (S)-4-ethyl-8-fluoro-4, 9-dihydroxy-1H-pyrano[3′, 4′:6, 7]indolizino[l, 2-b]quinoline-3, 14(4H, 12H)-dione (0.101 g, 0.264 mmol) in DCM (10 mL) on ice bath, DIPEA (0.050 mL, 0.287 mmol) and 4-nitrophenyl carbonochloridate (0.056 g, 0.279 mmol) were added. The mixture was then stirred at r. t. for 2 h, followed by addition of compound 213 (0.295 g, 0.136 mmol) and DIPEA (0.060 mL, 0.345 mmol). The reaction mixture was continued stirring for overnight, concentrated under reduced pressure and purified by silica gel column chromatography with a gradient of 5-15% methanol in DCM to give the title product (0.303 g, 76% yield). MS ESI m/z calcd. for C₁₄₀H₁₈₄F₂N₂₂O₄₈ [M+H]⁺ 2979.2601, found 2979.2890.

### Example 247. Synthesis of ((((11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioyl)bis(azanediyl))bis(4, 1-phenylene))bis(methylene) bis(((iS, 9S)-9-ethyl-5-fluoro-9-hydroxy-4-methyl-10, 13-dioxo-1, 2, 3, 9, 10, 12, 13, 15-octahydrobenzo[de]pyrano[3′, 4′:6, 7]-indolizino[1, 2-b]quinolin-1-yl)carbamate) (216)

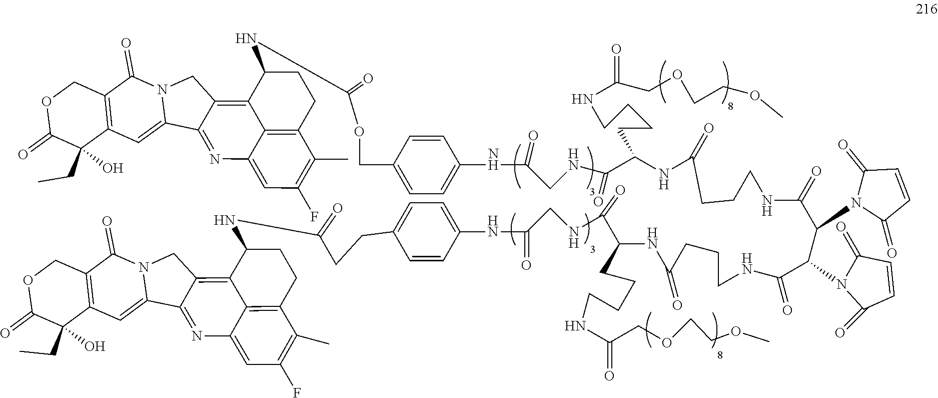

To a solution of compound 209 (0.201 g, 0.094 mmol) in CH₂CH₂ (5 mL) on ice bath, triphosgene (0.0575 g, 0.191 mmol) and DIPEA (0.040 mL, 0.230 mmol) were added under N₂. The mixture was stirred at 0° C. for 30 min and r. t. for 30 min, followed by addition of exatecan HCl salt (0.110 g, 0.233 mmol) and DIPEA (0.045 mL, 0.258 mmol) on ice bath. Then the mixture was stirred at r. t. for 2 h, concentrated under reduced pressure and purified by silica gel column chromatography with a gradient of 3-18% methanol in DCM to give the title product (0.207 g, 72% yield). MS ESI m/z calcd. for C₁₄₆H₁₉₀F₂N₂₂O₄₈ [M+H]⁺ 3058.3150, found 3058.3345.

### Example 248. Synthesis of (2R, 3S)-2, 3-bis(2, 5-dioxo-2, 5-dihydro-1H-pyrrol-1-yl)-N₁, N₄-bis((S)-34-((2-((2-(((2-(((1S, 9S)-9-ethyl-5-fluoro-9-hydroxy-4-methyl-10, 13-dioxo-1, 2, 3, 9, 10, 12, 13, 15-octahydrobenzo[de]pyrano[3′, 4′:6, 7]indolizino[1, 2-b]quinolin-1-yl)amino)-2-oxoethoxy)methyl)amino)-2-oxoethyl)amino)-2-oxoethyl)carbamoyl)-28, 36-dioxo-2, 5, 8, 11, 14, 17, 20, 23, 26-nonaoxa-29, 35-diazanonatriacontan-39-yl)succinamide (217)

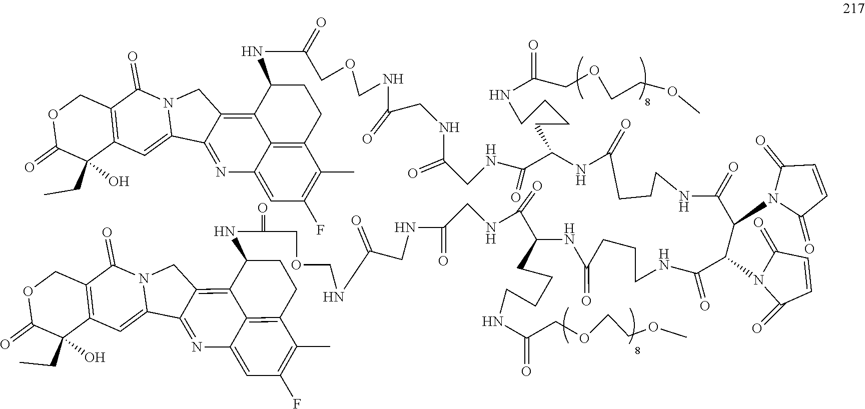

To a solution of (11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioic acid (0.301 g, 0.156 mmol), 2-(aminomethoxy)-N-((1S, 9S)-9-ethyl-5-fluoro-9-hydroxy-4-methyl-10, 13-dioxo-1, 2, 3, 9, 10, 12, 13, 15-octahydrobenzo[de]pyrano[3′, 4′:6, 7]indolizino[1, 2-b]quinolin-1-yl)acetamide (0.185 g, 0.354 mmol), EDC (0.150 g, 0.781 mmol) in DMA (8 mL), DIPEA (0.080 mL, 0.460 mmol) were added. Then the mixture was stirred at r. t. for 6 h, concentrated under reduced pressure and purified by silica gel column chromatography with a gradient of 3-18% methanol in DCM to give the title product (0.308 g, 70% yield). MS ESI m/z calcd. for C₁₃₂H₁₈₁F₂N₂₀O₄₆ [M+H]⁺ 2820.2408, found 2820.2635.

### Example 249. Synthesis of (2R, 3S)-2, 3-bis(2, 5-dioxo-2, 5-dihydro-1H-pyrrol-1-yl)-N₁, N₄-bis((S)-34-((1-((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)-3, 8, 11-trioxo-5-oxa-2, 7, 10-triazadodecan-12-yl)carbamoyl)-28, 36-dioxo-2, 5, 8, 11, 14, 17, 20, 23, 26-nonaoxa-29, 35-diazanonatriacontan-39-yl)succinamide (218)

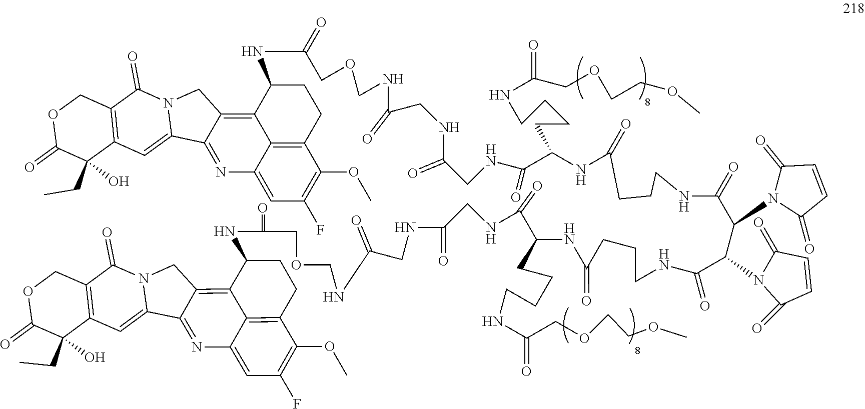

To a solution of (11S, 19R, 20S, 28S)-19, 20-bis(2, 5-dioxo-2, 5-dihydro-1H-pyrrol-1-yl)-4, 7, 10, 13, 18, 21, 26, 29, 32, 35-decaoxo-11, 28-bis(28-oxo-2, 5, 8, 11, 14, 17, 20, 23, 26-nonaoxa-29-azatritriacontan-33-yl)-3, 6, 9, 12, 17, 22, 27, 30, 33, 36-decaazaoctatriacontane-1, 38-dioic acid (0.251 g, 0.130 mmol), (S)-2-(aminomethoxy)-N-((4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)acetamide (0.158 g, 0.308 mmol), EDC (0.150 g, 0.781 mmol) in DMA (8 mL), DIPEA (0.070 mL, 0.402 mmol) was added. Then the mixture was stirred at r. t. for 6 h, concentrated under reduced pressure and purified by silica gel column chromatography with a gradient of 3-18% methanol in DCM to give the title product (0.265 g, 73% yield). MS ESI m/z calcd. for C₁₂₈H₁₇₇F₂N₂₀O₄₈ [M+H]⁺ 2800.1993, found 2800.2120.

### Example 250. Synthesis of Boc-N-Me-L-Val-OH (219)

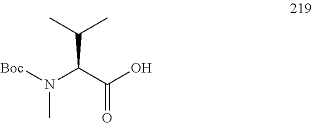 [SMILES: CC(C)[C@@H](C(=O)O)N(C)C(=O)OC(C)(C)C]

To a solution of Boc-L-Val-OH (2.00 g, 9.2 mmol) and methyl iodide (5.74 mL, 92 mmol) in anhydrous THF (40 mL) was added sodium hydride (3.68 g, 92 mmol) at 0° C. The reaction mixture was stirred at 0° C. for 1.5 h, then warmed to r.t. and stirred for 24 h. The reaction was quenched by ice water (50 mL). After addition of water (100 mL), the reaction mixture was washed with ethyl acetate (3×50 mL) and the aqueous solution was acidified to pH 3 then extracted with ethyl acetate (3×50 mL). The combined organic phase was dried over Na₂SO₄ and concentrated to afford Boc-N-Me-Val-OH (2.00 g, 94% yield) as a white solid. ¹H NMR (500 MHz, CDCl₃) δ 4.10 (d, J=10.0 Hz, 1H), 2.87 (s, 3H), 2.37-2.13 (m, 1H), 1.44 (d, J=26.7 Hz, 9H), 1.02 (d, J=6.5 Hz, 3H), 0.90 (t, J=8.6 Hz, 3H).

### Example 251. Synthesis of (S)-tert-butyl 2-((1R, 2R)-1-methoxy-3-(((S)-1-methoxy-1-oxo-3-phenylpropan-2-yl)amino)-2-methyl-3-oxopropyl)pyrrolidine-1-carboxylate (220)

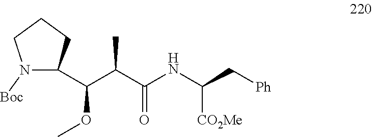 [SMILES: COC(=O)[C@H](Cc1ccccc1)NC(=O)[C@H](C)[C@@H](OC)[C@@H]1CCCN1C(=O)OC(C)(C)C]

To a solution of (2R, 3R)-3-((S)-1-(tert-butoxycarbonyl)pyrrolidin-2-yl)-3-methoxy-2-methylpropanoic acid (100 mg, 0.347 mmol) and L-phenylalanine methyl ester hydrochloride (107.8 mg, 0.500 mmol) in DMF (5 mL) at 0° C. was added diethyl cyanophosphonate (75.6 μL, 0.451 mmol), followed by Et₃N (131 μL, 0.94 mmol). The reaction mixture was stirred at 0° C. for 2 h, then warmed to r.t. and stirred overnight. The reaction mixture was then diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄, and concentrated in vacuo. The residue was purified by column chromatography (15-75% ethyl acetate/hexanes) to afford the title compound (130 mg, 83% yield) as a white solid. ¹H NMR (500 MHz, CDCl₃) δ 7.28 (dd, J=7.9, 6.5 Hz, 2H), 7.23 (t, J=7.3 Hz, 1H), 7.16 (s, 2H), 4.81 (s, 1H), 3.98-3.56 (m, 5H), 3.50 (s, 1H), 3.37 (d, J=2.9 Hz, 3H), 3.17 (dd, J=13.9, 5.4 Hz, 2H), 3.04 (dd, J=14.0, 7.7 Hz, 1H), 2.34 (s, 1H), 1.81-1.69 (m, 2H), 1.65 (s, 3H), 1.51-1.40 (m, 9H), 1.16 (d, J=7.0 Hz, 3H).

### Example 252. General Procedure for the Removal of the Boc Function with Trifluoroacetic Acid

To a solution of the N-Boc amino acid (1.0 mmol) in methylene chloride (2.5 mL) was added trifluoroacetic acid (1.0 mL). After being stirred at room temperature for 1-3 h, the reaction mixture was concentrated in vacuo. Co-evaporation with toluene gave the deprotected product, which was used without any further purification.

### Example 253. Synthesis of (S)-methyl 2-((2R, 3R)-3-((S)-1-((3R, 4S, 5S)-4-((tert-butoxycarbonyl)(methyl)amino)-3-methoxy-5-methylheptanoyl)pyrrolidin-2-yl)-3-methoxy-2-methylpropanamido)-3-phenylpropanoate (221)

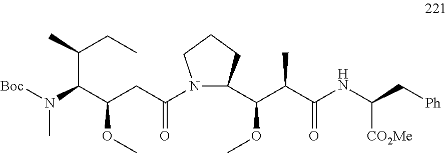 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)OC(C)(C)C]

To a solution of the Boc-deprotected product of (S)-tert-butyl 2-((1R, 2R)-1-methoxy-3-(((S)-1-methoxy-1-oxo-3-phenylpropan-2-yl)amino)-2-methyl-3-oxopropyl)pyrrolidine-1-carboxylate (0.29 mmol) and (3R, 4S, 5S)-4-((tert-butoxycarbonyl)(methyl)amino)-3-methoxy-5-methylheptanoic acid (96.6 mg, 0.318 mmol) in DMF (5 mL) at 0° C. was added diethyl cyanophosphonate (58 PL, 0.347 mmol), followed by Et₃N (109 μL, 0.78 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄ and concentrated in vacuo. The residue was purified by column chromatography (15-75% ethyl acetate/hexanes) to afford the title compound (150 mg, 81% yield) as a white solid. LC-MS (ESI) m/z calcd. for C₃₄H₅₅N₃Oₛ[M+H]⁺: 634.40, found: 634.40.

### Example 254. Synthesis of (S)-methyl 2-((2R, 3R)-3-((S)-1-((3R, 4S, 5S)-4-((S)-2-((tert-butoxycarbonyl)amino)-N, 3-dimethylbutanamido)-3-methoxy-5-methylheptanoyl)pyrrolidin-2-yl)-3-methoxy-2-methylpropanamido)-3-phenylpropanoate (222)

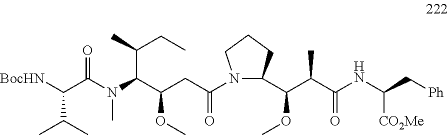 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)OC(C)(C)C)C(C)C]

To a solution of the Boc-deprotected product of (S)-methyl 2-((2R, 3R)-3-((S)-1-((3R, 4S, 5S)-4-((tert-butoxycarbonyl)(methyl)amino)-3-methoxy-5-methylheptanoyl)-pyrrolidin-2-yl)-3-methoxy-2-methylpropanamido)-3-phenylpropanoate (0.118 mmol) and Boc-Val-OH (51.8 mg, 0.236 mmol) in DCM (5 mL) at 0° C. was added BroP(70.1 mg, 0.184 mmol), followed by diisopropylethylamine (70 L, 0.425 mmol). The mixture was shielded from light and stirred at 0° C. for 30 min then at r.t. for 2 days. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over Na₂SO₄ and concentrated in vacuo. The residue was purified by column chromatography (20-100% ethyl acetate/hexanes) to afford the title compound (67 mg, 77% yield) as a white solid. LC-MS (ESI) m/z calcd. for C₃₉H₆₄N₄O₉ [M+H]⁺: 733.47, found: 733.46.

### Example 255. Synthesis of (S)-methyl 2-((2R, 3R)-3-((S)-1-((6S, 9S, 12S, 13R)-12-((S)-sec-butyl)-6, 9-diisopropyl-13-methoxy-2, 2, 5, 11-tetramethyl-4, 7, 10-trioxo-3-oxa-5, 8, 11-triazapentadecan-15-oyl)pyrrolidin-2-yl)-3-methoxy-2-methylpropanamido)-3-phenylpropanoate (223)

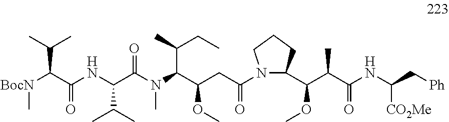 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)OC(C)(C)C)C(C)C]

To a solution of the Boc-deprotected product of (S)-methyl 2-((2R, 3R)-3-((S)-1-((3R, 4S, 5S)-4-((S)-2-((tert-butoxycarbonyl)amino)-N, 3-dimethylbutanamido)-3-methoxy-5-methylheptanoyl)pyrrolidin-2-yl)-3-methoxy-2-methylpropanamido)-3-phenylpropanoate (0.091 mmol) and Boc-N-Me-Val-OH (127 mg, 0.548 mmol) in DMF (5 mL) at 0° C. was added diethyl cyanophosphonate (18.2 μL, 0.114 mmol), followed by N-methylmorpholine (59 μL, 0.548 mmol). The reaction mixture was stirred at 0° C. for 2 h, then warmed to r.t. and stirred overnight. The reaction mixture was diluted with ethyl acetate (80 mL), washed with 1 N aqueous potassium hydrogen sulfate (40 mL), water (40 mL), saturated aqueous sodium hydrogen carbonate (40 mL), and saturated aqueous sodium chloride (40 mL), dried over sodium sulfate, and concentrated in vacuo. The residue was purified by column chromatography (20-100% ethyl acetate/hexanes) to afford the title compound (30 mg, 39% yield) as a white solid. LC-MS (ESI) m/z calcd. for C₄₅H₇₅N₅O₁₀ [M+H]⁺: 846.55, found: 846.56.

### Example 256. Synthesis of (S)-2-((2R, 3R)-3-((S)-1-((6S, 9S, 12S, 13R)-12-((S)-sec-butyl)-6, 9-diisopropyl-13-methoxy-2, 2, 5, 11-tetramethyl-4, 7, 10-trioxo-3-oxa-5, 8, 11-triazapenta-decan-15-oyl)pyrrolidin-2-yl)-3-methoxy-2-methylpropanamido)-3-phenylpropanoic acid (224)

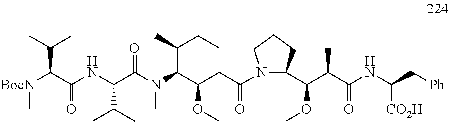 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OC(C)(C)C)C(C)C]

To a solution of (S)-methyl 2-((2R, 3R)-3-((S)-1-((6S, 9S, 12S, 13R)-12-((S)-sec-butyl)-6, 9-diisopropyl-13-methoxy-2, 2, 5, 11-tetramethyl-4, 7, 10-trioxo-3-oxa-5, 8, 11-triazapentadecan-15-oyl)pyrrolidin-2-yl)-3-methoxy-2-methylpropanamido)-3-phenylpropanoate (30 mg, 0.035 mmol) in THF (1.0 mL) was added LiOH in water (1.0M, 0.8 mL). The mixture was stirred at r.t. for 35 min, neutralized with 0.5 M H₃PO₄ to pH 6, concentrated and purified on silica gel column chromatography (CH₃OH/DCM/HOAc 1:10:0.01) to afford the title compound (25.0 mg, 85% yield). LC-MS (ESI) m/z calcd. for C₄₄H₇₄N₅O₁₀ [M+H]⁺: 832.54, found: 832.60.

### Example 257. Synthesis of (S)-2-((2R, 3R)-3-((S)-1-((3R, 4S, 5S)-4-((S)—N, 3-dimethyl-2-((S)-3-methyl-2-(methylamino)butanamido)butanamido)-3-methoxy-5-methylheptanoyl)-pyrrolidin-2-yl)-3-methoxy-2-methylpropanamido)-3-phenylpropanoic acid (MMAF) (225)

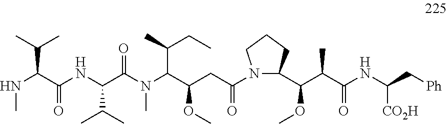 [SMILES: CC[C@H](C)C([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

(S)-Methyl 2-((2R, 3R)-3-((S)-1-((3R, 4S, 5S)-4-((S)—N, 3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate (25 mg, 0.030 mmol) in the mixture of conc. HCl (0.3 mL) and 1, 4-dioxane (0.9 mL) was stirred at r.t. for 35 min. The mixture was diluted with EtOH (1.0 mL) and toluene (1.0 mL), concentrated and co-evaporated with EtOH/toluene (2:1) to afford the title compound as a white solid (22 mg, ˜100% yield), which was used in the next step without further purification. LC-MS (ESI) m/z calcd. for C₃₉H₆₆N₅O₈ [M+H]⁺: 732.48, found: 732.60.

### Example 258. Synthesis of Compound 226

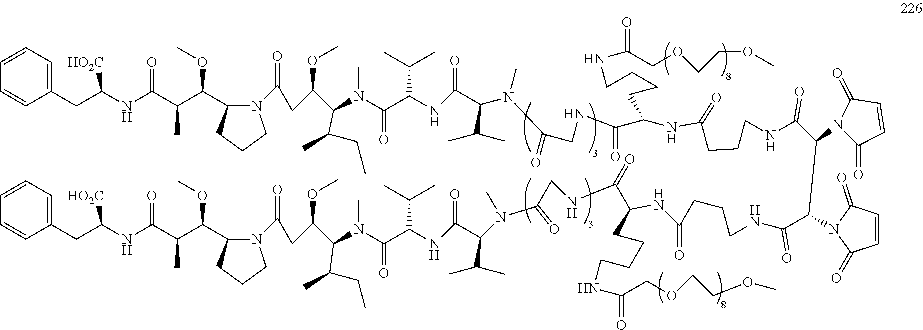

To a solution of compound 207 (0.101 g, 0.052 mmol), N-hydroxysucciminide (NHS) (0.020 g, 0.173 mmol), EDC (0.050 g, 0.260 mmol) in DMA (4 mL), DIPEA (0.020 mL, 0.115 mmol) was added. Then the mixture was stirred at r. t. for 6 h. Then the mixture was added to a solution of MMAF (0.095 g, 0.130 mmol) in DMA (1 mL) and NaH₂PO₄ (5 mL, 0.1 M, pH 7.5). The mixture was then stirred for 4 h, concentrated under reduced pressure and purified by C-18 HPLC chromatography (10 mL/min) with a gradient of methanol/H₂O (5%-50%) to give the title product (0.120 g, 69% yield) after lyophilization. MS ESI m/z calcd. for C₁₆₀H₂₆₃N₂₄O₅₂ [M+H]⁺ 3352.8674, found 3352.8935.

### Example 259. Synthesis of Compound 227

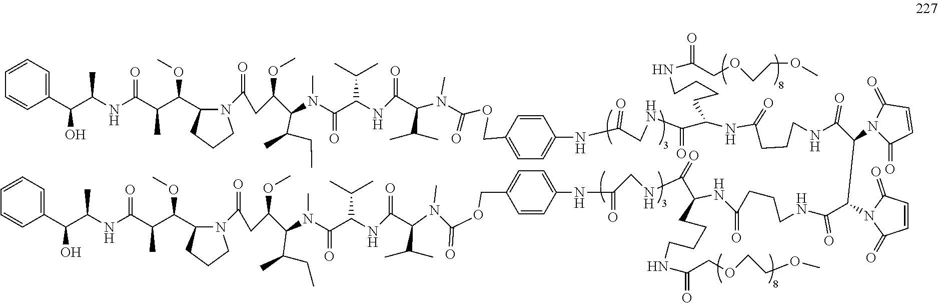

To a solution of compound 209 (0.101 g, 0.047 mmol) in CH₂CH₂ (5 mL) on ice bath, triphosgene (0.0285 g, 0.096 mmol) and DIPEA (0.020 mL, 0.115 mmol) were added under N₂. The mixture was stirred at 0° C. for 30 min and r. t. for 30 min, followed by addition of (S)—N-((3R, 4S, 5R)-1-((S)-2-((1R, 2R)-3-(((1S, 2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N, 3-dimethyl-2-((S)-3-methyl-2-(methylamino)butanamido) butanamide (MMAE) (0.080 g, 0.111 mmol) and DIPEA (0.025 mL, 0.144 mmol) on ice bath. Then the mixture was stirred at r. t. for 2 h, concentrated under reduced pressure and purified by silica gel column chromatography with a gradient of 3-18% methanol in DCM to give the title product (0.124 g, 73% yield). MS ESI m/z calcd. for C₁₇₆H₂₈₀N₂₇O₅₄ [M+H]⁺ 3623.0042, found 3623.0250.

### Example 260. Synthesis of (S)-methyl 2-((2R, 3R)-3-((S)-1-((3R, 4S, 5S)-4-((S)—N, 3-dimethyl-2-((S)-3-methyl-2-(methylamino)butanamido)butanamido)-3-methoxy-5-methyl-heptanoyl)pyrrolidin-2-yl)-3-methoxy-2-methylpropanamido)-3-phenylpropanoate (228)

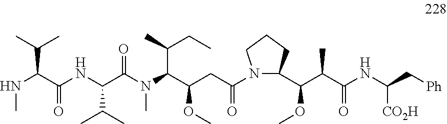 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

To a solution of (S)-methyl 2-((2R, 3R)-3-((S)-1-((6S, 9S, 12S, 13R)-12-((S)-sec-butyl)-6, 9-diisopropyl-13-methoxy-2, 2, 5, 11-tetramethyl-4, 7, 10-trioxo-3-oxa-5, 8, 11-triazapentadecan-15-oyl)pyrrolidin-2-yl)-3-methoxy-2-methylpropanamido)-3-phenylpropanoate (75.0 mg, 0.0886 mmol) in methylene chloride (5 mL) was added trifluoroacetic acid (2 mL) at room temperature. After being stirred at room temperature for 1 h, the reaction mixture was concentrated in vacuo. Co-evaporation with toluene gave the deprotected title product, which was used without further purification.

### Example 261. Synthesis of (S)-methyl 2-((2R, 3R)-3-((S)-1-((5S, 8S, 11S, 14S, 15R)-14-((S)-sec-butyl)-8, 11-diisopropyl-15-methoxy-5, 7, 13-trimethyl-3, 6, 9, 12-tetraoxo-1-phenyl-2-oxa-4, 7, 10, 13-tetraazaheptadecan-17-oyl)pyrrolidin-2-yl)-3-methoxy-2-methyl propanamido)-3-phenylpropanoate (229)

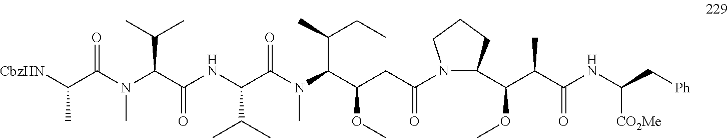 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)NC(=O)OCc1ccccc1)C(C)C]

To a solution of MMAF-OMe (0.132 g, 0.178 mmol, 1.0 eq.) and Z-L-Alanine (0.119 g, 0.533 mmol, 3.0 eq.) in anhydrous DCM (10 mL) at 0° C. was added HATU (0.135 g, 0.356 mmol, 2.0 eq.) and NMM (0.12 mL, 1.07 mmol, 6.0 eq.) in sequence. The reaction was stirred at 0° C. for 10 minutes, then warmed to room temperature and stirred overnight. The mixture was diluted with DCM and washed with water and brine, dried over anhydrous Na₂SO₄, concentrated and purified by silica gel column chromatography (20:1 DCM/MeOH) to give the title compound as a white foamy solid (0.148 g, 88% yield). ESI MS m/z: calcd. for C₅₁H₇₉N₆O₁₁ [M+H]⁺ 951.6, found 951.6.

### Example 262. Synthesis of (S)-methyl 2-((2R, 3R)-3-((S)-1-((3R, 4S, 5S)-4-((S)-2-((S)-2-((S)-2-amino-N-methylpropanamido)-3-methylbutanamido)-N, 3-dimethylbutanamido)-3-methoxy-5-methylheptanoyl)pyrrolidin-2-yl)-3-methoxy-2-methylpropanamido)-3-phenylpropanoate (230)

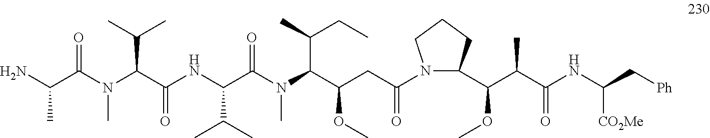 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C(=O)[C@H](C)N)C(C)C]

To a solution of (S)-methyl 2-((2R, 3R)-3-((S)-1-((5S, 8S, 11S, 14S, 15R)-14-((S)-sec-butyl)-8, 11-diisopropyl-15-methoxy-5, 7, 13-trimethyl-3, 6, 9, 12-tetraoxo-1-phenyl-2-oxa-4, 7, 10, 13-tetraazaheptadecan-17-oyl)pyrrolidin-2-yl)-3-methoxy-2-methylpropan amido)-3-phenyl-propanoate (0.148 g, 0.156 mmol, 1.0 eq.uiv) in MeOH (5 mL) was added Pd/C (0.100 g, 10% Pd/C, 50% wet) in a hydrogenation bottle. The mixture was shaken for 5 h then filtered through a Celite pad. The filtrate was concentrated to give the title compound as a white foamy solid (0.122 g, 96% yield). ESI MS m/z: calcd. for C₄₃H₇₃N₆O₉ [M+H]⁺ 817.5, found 817.5.

### Example 263. Synthesis of Compound 231

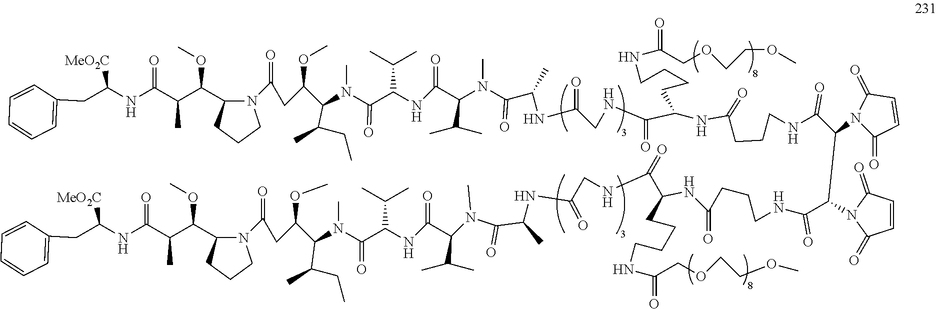

To a solution of compound 207 (0.101 g, 0.052 mmol), compound 230 (0.106 g, 0.130 mmol), EDC (0.100 g, 0.521 mmol) in DMA (4 mL), DIPEA (0.040 mL, 0.230 mmol) was added. Then the mixture was stirred at r. t. for 6 h, concentrated under reduced pressure and purified by silica gel column chromatography with a gradient of 3-15% methanol in DCM to give the title product (0.135 g, 74% yield). MS ESI m/z calcd. for C₁₆₈H₂₇₇N₂₆O₅₄ [M+H]⁺ 3522.9729, found 3522.9980.

### Example 264. Synthesis of (5S,12S,13S,20S)-di-tert-butyl 12,13-bis(((benzyloxy)-carbonyl)amino)-4,7,11,14,18,21-hexaoxo-5,20-bis(4-(((2,2,2-trichloroethoxy)carbonyl)amino)-butyl)-3,6,10,15,19,22-hexaazatetracosane-1,24-dioate (232)

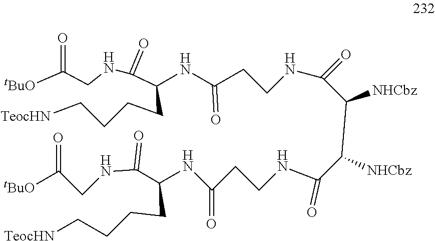

To a solution of (S)-tert-butyl 2-(2-(3-aminopropanamido)-6-(((2,2,2-trichloroethoxy)-carbonyl)amino)hexanamido)acetate (6.05 g, 12.0 mmol) and (2S,3S)-2,3-bis(((benzyloxy)-carbonyl)amino)succinic acid (2.48 g, 5.96 mmol) in DMA (60 mL), EDC·HCl (5.01 g, 26.09 mmol) and DIPEA (4.7 mL, 26.4 mmol) were added. The reaction mixture was stirred at r.t. overnight, then diluted with 150 mL dichloromethane and poured into a separatory funnel containing 100 mL of water. The organic phase was separated, washed with brine (2×80 mL), dried over anhydrous sodium sulfate, filtered and concentrated. The residue was purified by column chromatography (10-80% ethyl acetate/petroleum ether) to afford the title compound 232 (7.03 g, 85% yield). ESI MS m/z 1389.50 ([M+H]⁺).

### Example 265. Synthesis of (5S,12S,13S,20S)-di-tert-butyl 12,13-diamino-4,7,11,14,18,21-hexaoxo-5,20-bis(4-(((2,2,2-trichloroethoxy)carbonyl)amino)butyl)-3,6,10,15,19,22-hexaazatetracosane-1,24-dioate (233)

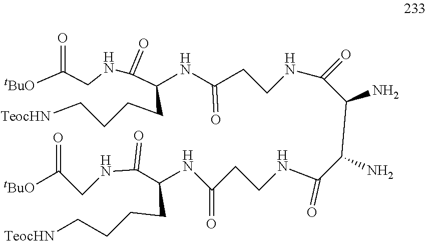

To a solution of compound 232 (7.01 g, 5.02 mmol) in methanol (100 mL) was added Pd/C (10 wt %, 0.80 g) in a hydrogenation bottle. The mixture was shaken for 2 h, filtered through Celite (filter aid), and the filtrate was concentrated to afford compound 233 (5.57 g, 99% yield) as a colorless oil. ESI MS m/z 1121.55 ([M+H]⁺).

### Example 266. Synthesis of (5S,12S,13S,20S)-di-tert-butyl 12,13-bis(2,5-dioxo-2,5-dihydro-1H-pyrrol-1-yl)-4,7,11,14,18,21-hexaoxo-5,20-bis(4-(((2,2,2-trichloroethoxy)carbonyl)amino)butyl)-3,6,10,15,19,22-hexaazatetracosane-1,24-dioate (234)

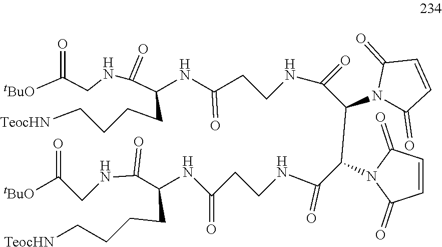

To a solution of compound 233 (5.49 g, 4.90 mmol) in saturated solution of NaHCO₃ (90 mL) and MeOH (10 mL) cooled at 0° C., N-(methoxycarbonyl)maleimide (3.10 g, 20.00 mmol) was added to the stirred solution. After 20 mins the reaction mixture was diluted with water (150 mL) and stirred for 30 min at room temperature. The reaction mixture was concentrated at 2-8° C. to ˜100 mL and extracted with DCM (4×60 mL). The organic layers were combined, dried over MgSO₄, filtered, concentrated and purified by silica gel column chromatography with a gradient of 5-10% methanol in DCM to give the title product 234 (4.893 g, 78% yield). MS ESI [M+H]⁺ 1281.55.

### Example 267. Synthesis of (5S,12S,13S,20S)-12,13-bis(2,5-dioxo-2,5-dihydro-1H-pyrrol-1-yl)-4,7,11,14,18,21-hexaoxo-5,20-bis(4-(((2,2,2-trichloroethoxy)carbonyl)amino)butyl)-3,6,10,15,19,22-hexaazatetracosane-1,24-dioic acid (235)

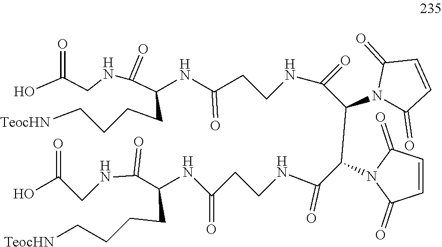

A solution of compound 234 (4.88 g, 3.81 mmol) in dioxane (15 mL) was treated with 4 N HCl (5 mL) at 0° C. for 30 min, diluted with toluene (10 ml) then concentrated, and purified with a short silica gel chromatography eluted with 0-18% methanol/dichloromethane to give a colorless oil (4.01 g, 90% yield). ESI MS m/z 1169.25 ([M+H]⁺).

### Example 268. Synthesis of Compound 236

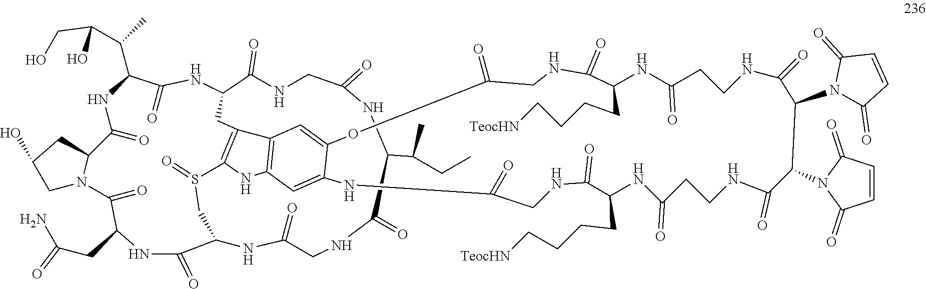

To a solution of compound 235 (130.0 mg, 0.111 mmol) and an amanitin derivative (104.0 mg, 0.111 mmol, WO2020/155017) in DMF (10 mL), TBTU (140.6 mg, 0.442 mmol), DIPEA (40.0 μL, 0.229 mmol) were added and the mixture was stirred at r.t. for 4 h. After removal of DMF under high vacuum, the residue was purified by C-18 prep-HPLC (acetonitrile/water, 5%-50% MeCN/H2O in 45 min, d20×250 mm, 10 ml/min) to give a colorless oil (133.2 mg, 58% yield). ESI MS m/z 2066.70 ([M+H]⁺).

### Example 269. Synthesis of Compound 237

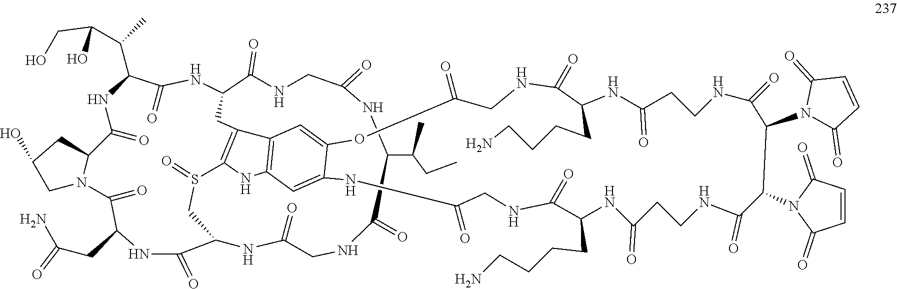 [SMILES: CC[C@H](C)[C@@H]1NC(=O)CNC(=O)[C@@H]2Cc3c([nH]c4cc5c(cc34)OC(=O)CNC(=O)[C@H](CCCCN)NC(=O)CCNC(=O)[C@@H](N3C(=O)C=CC3=O)[C@H](N3C(=O)C=CC3=O)C(=O)NCCC(=O)N[C@@H](CCCCN)C(=O)NCC(=O)N5)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@H](O)CO)C(=O)N2]

A solution of compound 236 (120.0 mg, 0.058 mmol) in THF (10 mL) was treated with TBAF (1.0 M in THF, 350 μL) at 0° C. for 30 min, then concentrated and purified purified by C-18 prep-HPLC (acetonitrile/water, 5%-40% MeCN/H2O in 45 min, d20×250 mm, 10 ml/min) to give 237 as oil (79.2 mg, 79% yield). ESI MS m/z 1718.85 ([M+H]⁺).

### Example 270. Synthesis of 14-(benzyloxy)-14-oxotetradecanoic acid (238)

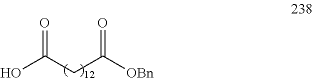

To a solution of tetradecanedioic acid (2.06 g, 8 mmol) in DMF (30 mL), K₂CO₃ (1.1 g, 8 mmol) and BnBr (1.36 g, 8 mmol) were added. The mixture was stirred at r.t. overnight, then concentrated and purified by column chromatography (ethyl acetate/petroleum ether) to afford the title compound 238 (1.2 g, 45% yield). ESI MS m/z 349.23 ([M+H]⁺).

### Example 271. Synthesis of tert-butyl 3-(2-(2-(2-hydroxyethoxy)ethoxy)ethoxy) propanoate (239)

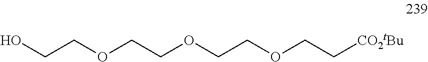 [SMILES: CC(C)(C)OC(=O)CCOCCOCCOCCO]

To a solution of 2, 2′-(ethane-1, 2-diylbis(oxy))diethanol (55.0 mL, 410.75 mmol, 3.0 eq.) in anhydrous THF (200 mL), sodium (0.1 g) was added. The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THF was removed by rotary evaporation, brine (300 mL) was added and the resulting mixture was extracted with ethyl acetate (3×100 mL). The organic layers were washed with brine (3×300 mL), dried over anhydrous sodium sulfate, filtered and concentrated to afford a colorless oil of the title compound (30.20 g, 79.0% yield), which was used without further purification. MS ESI m/z 278.17 ([M+H]⁺).

### Example 272. Synthesis of tert-butyl 3-(2-(2-(2-(tosyloxy)ethoxy)ethoxy)ethoxy) propanoate (240)

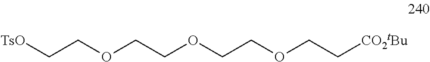 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C., TEA (30.0 mL, 217.0 mmol, 2.0 eq.) was added. The mixture was stirred at room temperature overnight, and then washed with water (3×300 mL) and brine (300 mL), dried over anhydrous sodium sulfate, filtered, concentrated and purified by silica gel column chromatography (3:1 hexanes/ethyl acetate) to give a colorless oil (39.4 g, 84.0% yield). MS ESI m/z 433.28 ([M+H]⁺).

### Example 273. Synthesis of tert-butyl 3-(2-(2-(2-azidoethoxy)ethoxy)ethoxy) propanoate (241)

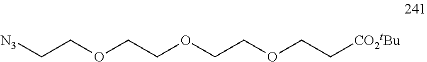 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF(100 mL), NaN₃ (20.67 g, 316.6 mmol, 3.5 eq.) was added. The mixture was stirred at room temperature overnight. Water (500 mL) was added and extracted with ethyl acetate (3×300 mL). The combined organic layers were washed with water (3×900 mL) and brine (900 mL), dried over anhydrous sodium sulfate, filtered, concentrated and purified by silica gel column chromatography (5:1 hexanes/ethyl acetate) to give a light-yellow oil (23.8 g, 85.53% yield). MS ESI m/z 326.2 ([M+Na]⁺).

### Example 274. Synthesis of tert-butyl 3-(2-(2-(2-aminoethoxy)ethoxy)ethoxy) propanoate (242)

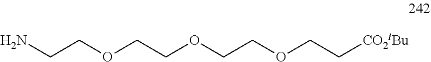 [SMILES: CC(C)(C)OC(=O)CCOCCOCCOCCN]

Raney-Ni (7.5 g, suspended in water) was washed with water (three times) and isopropyl alcohol (three times) and mixed with compound 241 (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% methanol/dichloromethane) to give a light-yellow oil (2.60 g, 57% yield). MS ESI m/z 279.19 ([M+H]⁺).

### Example 275. Synthesis of 27-benzyl 1-tert-butyl 14-oxo-4, 7, 10-trioxa-13-azaheptacosane-1, 27-dioate (243)

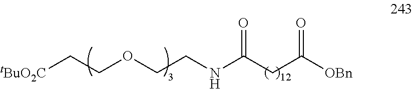

To a solution of compound 238 (2.60 g, 9.35 mmol) and compound 242 (3.91 g, 11.2 mmol) in dichloromethane (50 mL), EDC·HCl (2.15 g, 11.2 mmol) and DIPEA (3.6 mL, 20.6 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound (4.94 g, 87% yield). ESI m/z 608.40 ([M+H]⁺).

### Example 276. Synthesis of 3, 16-dioxo-1-phenyl-2, 20, 23, 26-tetraoxa-17-azanonacosan-29-oic acid (244)

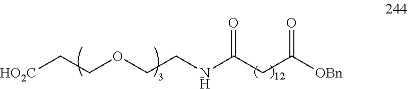

To a solution of compound 243 (4.94 g, 8.14 mmol) in dichloromethane (20 mL), TFA (20 mL) was added. The reaction was stirred at room temperature for 1 h, then concentrated to dryness and co-evaporated twice with dichloromethane, and the residue was placed on a pump to give compound 244 (4.50 g, crude product). ESI MS m/z 552.35 ([M+H]⁺).

### Example 277. Synthesis of 40-benzyl 1-tert-butyl 14, 27-dioxo-4, 7, 10, 17, 20, 23-hexaoxa-13, 26-diazatetracontane-1, 40-dioate (245)

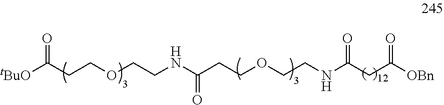

To a solution of compound 244 (4.50 g, crude, 8.14 mmol) and compound 242 (1.95 g, 7.00 mmol) in dichloromethane (50 mL), EDC·HCl (1.56 g, 8.14 mmol) and DIPEA (2.7 mL, 15.4 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 245 (5.22 g, 92% yield). ESI m/z 811.52 ([M+H]⁺).

### Example 278. Synthesis of 3, 16, 29-trioxo-1-phenyl-2, 20, 23, 26, 33, 36, 39-heptaoxa-17, 30-diazadotetracontan-42-oic acid (246)

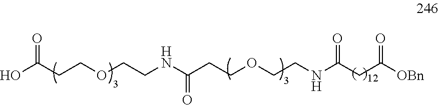

To a solution of compound 245 (5.22 g, 6.44 mmol) in dichloromethane (20 mL), TFA (5 mL) was added. The reaction was stirred at room temperature for 1 h, then concentrated to dryness and co-evaporated twice with dichloromethane, and the residue was placed on a pump to give compound 246 (4.90 g, crude product). ESI MS m/z 755.46 ([M+H]⁺).

### Example 279. Synthesis of 40-benzyl 1-(2, 5-dioxopyrrolidin-1-yl) 14, 27-dioxo-4, 7, 10, 17, 20, 23-hexaoxa-13, 26-diazatetracontane-1, 40-dioate (247)

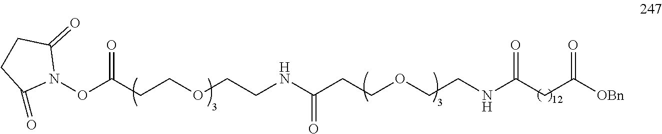

To a solution of compound 246 (4.90 g, crude, 6.44 mmol) in dichloromethane (30 mL), NHS (0.81 g, 7.08 mmol), EDC·HCl (1.85 g, 9.66 mmol), and DIPEA (2.8 mL, 16.1 mmol) were added. The reaction mixture was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil 247 (4.90 g, 90% yield). ESI MS m/z 852.48 ([M+H]⁺).

### Example 280. Synthesis of 1-((2, 5-dioxopyrrolidin-1-yl)oxy)-1, 14, 27-trioxo-4, 7, 10, 17, 20, 23-hexaoxa-13, 26-diazatetracontan-40-oic acid (248)

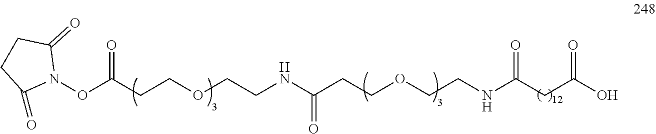

To a solution of compound 247 (4.90 g, 5.75 mmol) in THF (20 mL) in a hydrogenation bottle, Pd/C (10 wt %, 0.20 g) was added. The mixture was stirred under 1 atm H₂ overnight, filtered through Celite (filter aid), and the filtrated solution was concentrated to afford compound 248 (4.50 g, >100% yield). ESI MS m/z 762.44 ([M+H]⁺).

### Example 281. Synthesis of Compound 249

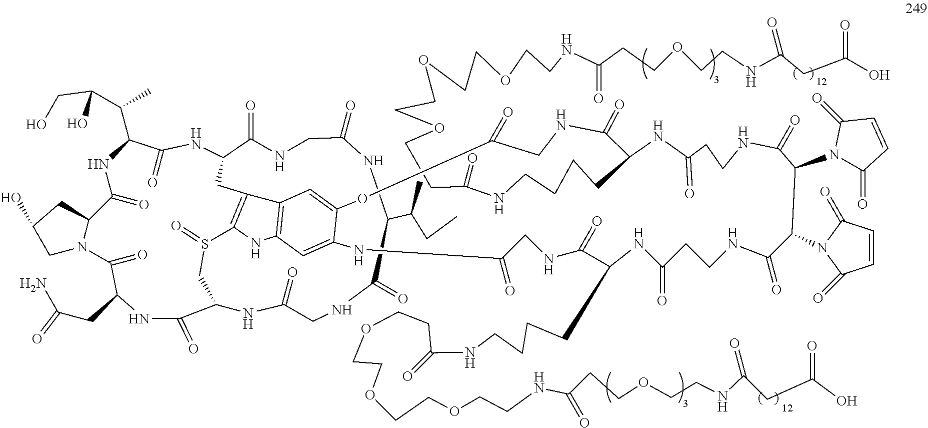

A mixture of compound 237 (60.2 mg, 0.0349 mmol) and compound 248 (110.2 mg, 0.146 mmol) in THF (10 mL) and phosphate buffer solution (10 mL, 0.2 M, pH 7.7) was stirred at r.t. overnight, then concentrated and purified by C-18 prep-HPLC (acetonitrile/water, 5%-40% MeCN/H₂O in 45 min, d20×250 mm, 10 ml/min) to give a white foam (80.2 mg, 76% yield). ESI MS m/z 3011.65 ([M+H]⁺).

### Example 282. Synthesis of 4-(benzyloxy)-3-methoxybenzoic acid (250)

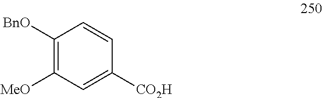 [SMILES: COc1cc(C(=O)O)ccc1OCc1ccccc1]

To a mixture of 4-hydroxy-3-methoxybenzoic acid (50.0 g, 297.5 mmol) in ethanol (350 mL) and aq. NaOH solution (2.0 M, 350 mL) was added BnBr (140.0 g, 823.5 mmol). The mixture was stirred at 65° C. for 8 h, concentrated, co-evaporated with water (2×400 mL) and concentrated to ˜400 mL, acidified to pH 3.0 with 6 N HCl. The solid was collected by filtration, crystallized with EtOH, dried at 45° C. under vacuum to afford the title compound (63.6 g, 83% yield). ESI MS m/z 281.2 ([M+Na]⁺).

### Example 283. Synthesis of 4-(benzyloxy)-5-methoxy-2-nitrobenzoic acid (251)

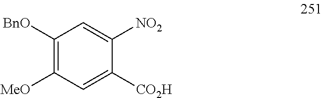 [SMILES: COc1cc(C(=O)O)c([N+](=O)[O-])cc1OCc1ccccc1]

To a solution of 4-(benzyloxy)-3-methoxybenzoic acid (63.5 g, 246.0 mmol) in DCM (400 mL) and HOAc (100 mL) was added HNO₃ (fuming, 25.0 mL, 528.5 mmol). The mixture was stirred for 6 h, concentrated, crystallized with EtOH, dried at 40° C. under vacuum to afford the title compound (63.3 g, 85% yield). ESI MS m/z 326.1 ([M+Na]⁺).

### Example 284. Synthesis of (S)-(4-(benzyloxy)-5-methoxy-2-nitrophenyl)(2-(hydroxyl methyl)-4-methylenepyrrolidin-1-yl)methanone (252)

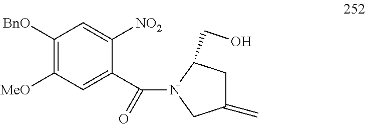 [SMILES: C=C1C[C@@H](CO)N(C(=O)c2cc(OC)c(OCc3ccccc3)cc2[N+](=O)[O-])C1]

A catalytic amount of DMF (30 p1) was added to a solution of 4-(benzyloxy)-5-methoxy-2-nitrobenzoic acid (2.70 g, 8.91 mmol) and oxalyl chloride (2.0 mL, 22.50 mmol) in anhydrous DCM (70 mL) and the resulting mixture was stirred at room temperature for 2 h. Excess DCM and oxalyl chloride was removed with rotavap. The acetyl chloride was re-suspended in fresh DCM (70 mL) and was added slowly to a pre-mixed solution of (S)-(4-methylenepyrrolidin-2-yl)methanol, hydrochloride salt (1.32 g, 8.91 mmol) and Et₃N (6 mL) in DCM at 0° C. under N₂ atmosphere. The reaction mixture was allowed to warm to r.t. and stirring was continued for 8 h. After removal of DCM and Et₃N, the residue was partitioned between H₂O and EtOAc (70/70 mL). The aqueous layer was further extracted with EtOAc (2×60 mL). The combined organic layers were washed with brine (40 mL), dried (MgSO₄) and concentrated. Purification of the residue with flash chromatography (silica gel, 2:8 hexanes/EtOAc) yielded the title compound (2.80 g, 79% yield). EI MS m/z 421.2 ([M+Na]⁺).

### Example 285. Synthesis of (S)-(4-(benzyloxy)-5-methoxy-2-nitrophenyl)(2-(((tert-butyldimethylsilyl)oxy)methyl)-4-methylenepyrrolidin-1-yl)methanone (253)

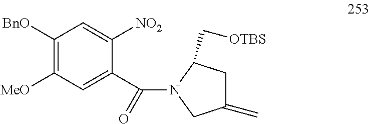 [SMILES: C=C1C[C@@H](CO[Si](C)(C)C(C)(C)C)N(C(=O)c2cc(OC)c(OCc3ccccc3)cc2[N+](=O)[O-])C1]

(S)-(4-(Benzyloxy)-5-methoxy-2-nitrophenyl)(2-(hydroxymethyl)-4-methylene pyrrolidin-1-yl)methanone (2.78 g, 8.52 mmol) in the mixture of DCM (10 mL) and pyridine (10 mL) was added tert-butylchlorodimethylsilane (2.50 g, 16.66 mmol). The mixture was stirred for overnight, concentrated and purified on silica gel column eluted with EtOAc/DCM (1:6) to afford the title compound (3.62 g, 83% yield, ˜95% pure). MS ESI m/z calcd. for C₂₇H₃₇N₂O₆Si [M+H]⁺ 513.23, found 513.65.

### Example 286. Synthesis of (S)-(4-hydroxy-5-methoxy-2-nitrophenyl) (2-(hydroxyl methyl)-4-methylenepyrrolidin-1-yl)methanone (254)

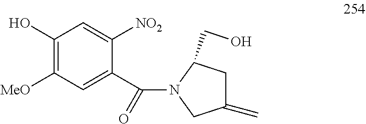 [SMILES: C=C1C[C@@H](CO)N(C(=O)c2cc(OC)c(O)cc2[N+](=O)[O-])C1]

(S)-(4-(Benzyloxy)-5-methoxy-2-nitrophenyl)(2-(hydroxymethyl)-4-methylene pyrrolidin-1-yl)methanone (2.80 g, 7.03 mmol) in the mixture of DCM (30 mL) and CH₃SO₃H (8 mL) was added PhSCH₃ (2.00 g, 14.06 mmol). The mixture was stirred for 0.5 h, diluted with DCM (40 mL), neutralized with carefully addition of 0.1 M Na₂CO₃ solution. The mixture was separated and the aqueous solution was extracted with DCM (2×10 mL). The organic layers were combined, dried over Na₂SO₄, concentrated and purified on silica gel column eluted with MeOH/DCM (1:15 to 1:6) to afford the title compound (1.84 g, 85% yield, ˜95% pure). MS ESI m/z calcd. for C₁₄H₁₇N₂O₆ [M+H]⁺ 309.10, found 309.30.

### Example 287. Synthesis of (S)-((pentane-1, 5-diylbis(oxy))bis(5-methoxy-2-nitro-4, 1-phenylene))bis(((S)-2-(hydroxymethyl)-4-methylenepyrrolidin-1-yl)methanone) (255)

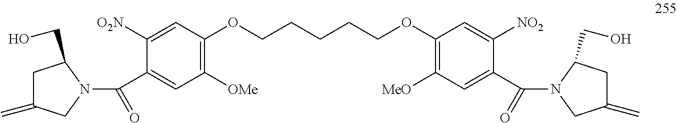

To a solution of compound 254 (0.801 g, 2.60 mmol) in butanone (10 mL) was added Cs₂CO₃, (2.50 g, 7.67 mmol), followed by addition of 1, 5-diiodopentane (415 mmol, 1.28 mmol). The mixture was stirred for 26 h, concentrated and purified on silica gel column eluted with MeOH/DCM (1:15 to 1:5) to afford the title compound (0.675 g, 77% yield, ˜95% pure). MS ESI m/z calcd. for C₃₃H₄₁N₄O₁₂ [M+H]⁺ 685.26, found 685.60.

### Example 288. Synthesis of (S)-((pentane-1, 5-diylbis(oxy))bis(2-amino-5-methoxy-4, 1-phenylene))bis(((S)-2-(hydroxymethyl)-4-methylenepyrrolidin-1-yl)methanone) (256)

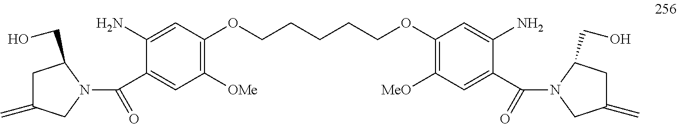

To a solution of compound 255 (0.670 g, 0.98 mmol) in CH₃OH (10 mL) was added Na₂S204 (1.01 g, 5.80 mmol) in H₂O (8 mL). The mixture was stirred at room temperature for 30 h. The reaction mixture was evaporated and co-evaporated with DMA (2×10 mL) and EtOH (2×10 mL) under high vacuum to dryness to afford the title compound (total weight 1.63 g) containing inorganic salts which was used directly for the next step reaction (without further separation). EIMS m/z 647.32 ([M+Na]⁺).

### Example 289. Synthesis of 257

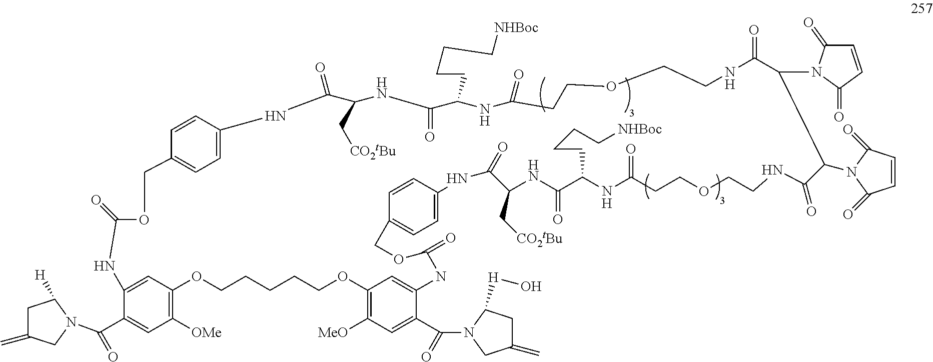

To a solution of (3S, 6S, 39S, 42S)-di-tert-butyl 6, 39-bis(4-((tert-butoxy carbonyl)amino)butyl)-22, 23-bis(2, 5-dioxo-2, 5-dihydro-1H-pyrrol-1-yl)-3, 42-bis((4-(hydroxymethyl)phenyl)carbamoyl)-5, 8, 21, 24, 37, 40-hexaoxo-1 1, 14, 17, 28, 31, 34-hexaoxa-4, 7, 20, 25, 38, 41-hexaazatetratetracontane-1, 44-dioate (0.840 g, 0.488 mmol) in THF (8 mL) containing pyridine (0.100 mL, 1.24 mmol) at 0° C. was added dropwise a solution of triphosgene (0.290 mg, 0.977 mmol) in THF (3.0 mL). The reaction mixture was stirred at 0° C. for 15 min then was used directly in the next step.

To a suspension of compound 256 (0.842 mg, ˜0.49 mmol, containing inorganic salts) in EtOH (10 mL) at 0° C. was added the chloride prepared above. The mixture was stirred at 0° C. for 4 h, then warmed to r. t. for 1 h, concentrated, and purified by reverse phase HPLC (250 (L) mm×10(d) mm, C₁₈ column, 10-80% acetonitrile/water in 40 min v=8 mL/min) to afford the title compound (561.1 mg, 48% yield in three steps). ESI MS m/z: calcd. for C₁₁₇H₁₆₃N₁₆O₃₈ [M+H]⁺2400.12, found 2400.90.

### Example 290. Synthesis of 258

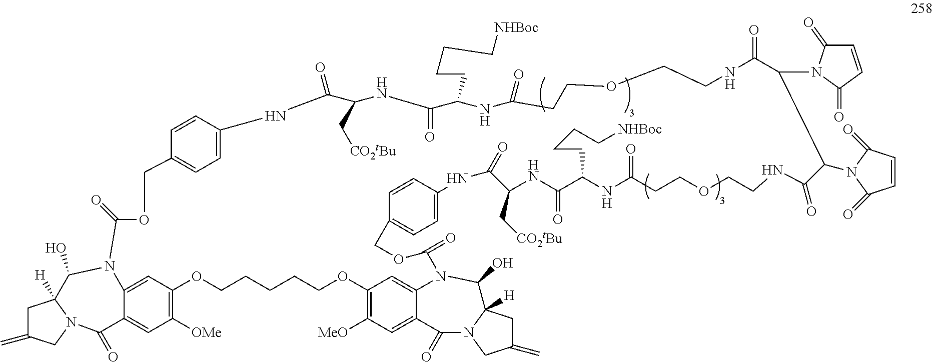

Dess-Martin periodinane (138.0 mg, 0.329 mmol) was added to a solution of compound 257 (132.0 mg, 0.055 mmol) in DCM (5.0 mL) at 0° C. The reaction mixture was warmed to r. t. and was stirred for 2 h. A saturated solution of NaHCO₃/Na₂SO₃(5.0 mL/5.0 mL) was then added and the mixture was extracted with DCM (3×25 mL). The combined organic layers were washed with NaHCO₃/Na₂SO₃ (5.0 mL/5.0 mL), brine (10 mL), dried over Na₂SO₄, filtered, concentrated and purified by reverse phase HPLC (250 (L) mmx 10(d) mm, C₁₈ column, 10-80% acetonitrile/water in 40 min, v=8 mL/min) to afford the title compound (103.1 mg, 78% yield) as a foam. ESI MS m/z: calcd. for C₁₁₇H₁₅₈N₁₆O₃₈ [M+H]⁺ 2396.09, found 2396.65.

### Example 291. Synthesis of 259

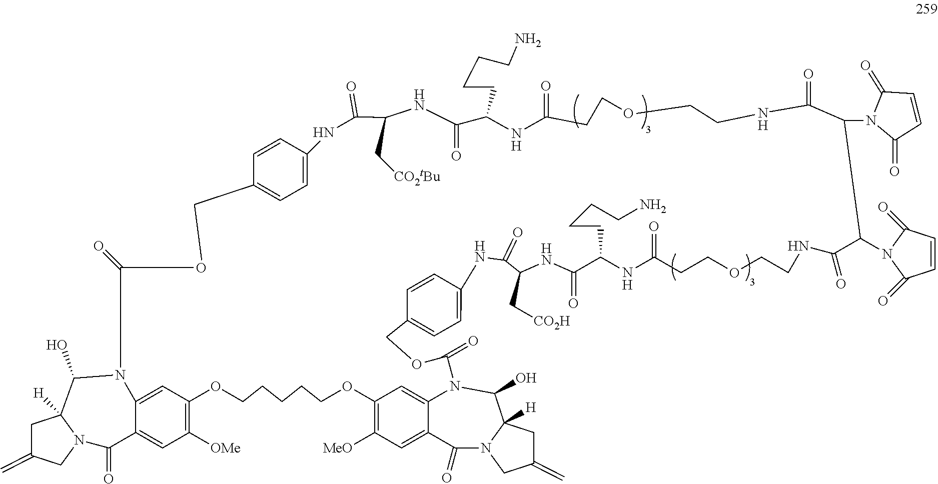

Compound 258 (55.0 mg, 0.023 mmol) was dissolved in DCM (3 mL), and TFA (3 mL) was added at 4° C. The reaction mixture was then stirred at r.t. for 1 h, then concentrated, and co-evaporated with DCM/toluene to dryness to afford the crude product C-3 (48.0 mg, 100% yield, 92% pure by HPLC) which was further purified by reverse phase HPLC (250 (L) mm×20(d) mm, C₁₈ column, 5-60% acetonitrile/water in 40 min, v=8 mL/min) to afford the pure product C-3 (42.1 mg, 88% yield, 96% pure) as a foam. ESI MS m/z: calcd. for C₉₉H₁₂₆N₁₆O₃₄ [M+H]⁺ 2083.86, found 2084.35.

### Example 292. Synthesis of 260

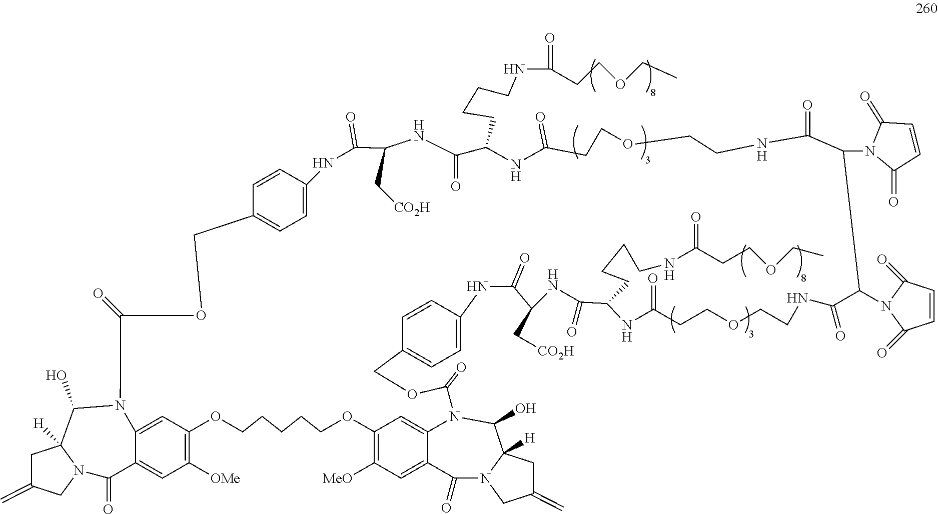

Compound 259 (35.0 mg, 0.017 mmol) was dissolved in a mixture solution of THF (3 mL) and 0.1 M, NaH₂PO₄ (3 mL), pH 7.5, and N-succinimidyl 2, 5, 8, 11, 14, 17, 20, 23-octaoxahexacosan-26-oate (43.0 mg, 0.084 mmol) was added in 4 portions in 2 h. The reaction mixture was then continued to stir at r.t. for 4 h, and co-evaporated with DMF (10 mL) to dryness to afford the crude product which was further purified by reverse phase HPLC (250 (L) mm×20(d) mm, C₁₈ column, 20-60% acetonitrile/water in 40 min, v=8 mL/min) to afford the pure product 260 (39.4 mg, 81% yield, 96% pure) as a foam. ESI MS m/z: calcd. for C₁₃₅H₁₉₅N₁₆O₅₂ [M+H]⁺ 2872.30, found 2871.65.

### Example 293. Synthesis of 261

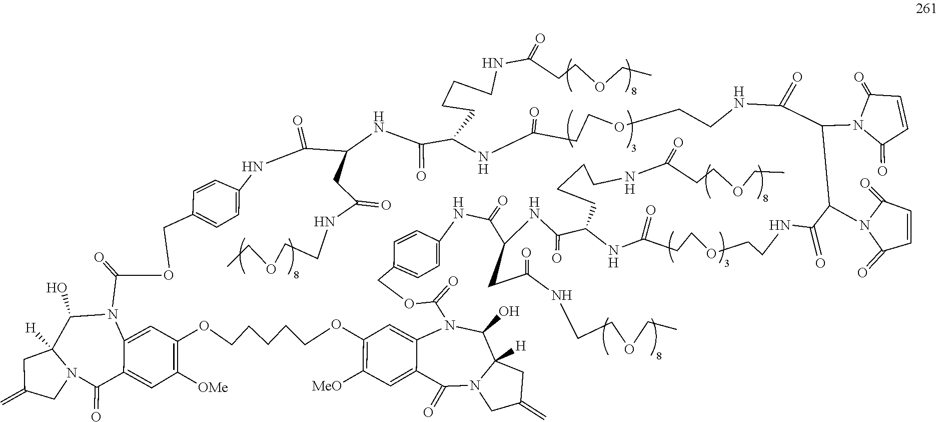

To a solution of compound 260 (35.0 mg, 0.012 mmol) and 2, 5, 8, 11, 14, 17, 20, 23-octaoxapentacosan-25-amine (15.1 mg, 0.0394 mmol) in dry DMA (2 mL) was added EDC (30.0 mg, 0.156 mmol). The reaction mixture was stirred at r.t. for 14 h, concentrated, purified by reverse phase HPLC (250 (L) mm×20(d) mm, C₁₈ column, 20-60% acetonitrile/water in 40 min, v=8 mL/min) to afford the pure product 261 (31.2 mg, 77% yield, 97% pure by HPLC) as a foam. ESI MS m/z: calcd. for C₁₆₁H₂₄₉N₁₈O₆₂ [M+H]⁺ 3426.68, found 3427.21.

### Example 294. General Synthesis of tert-butyl 3-(ω-methoxyl PEGyl)propanoate

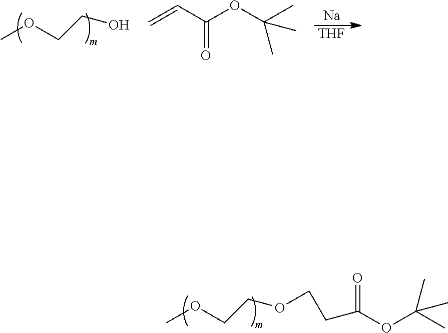

A PEG 1 eq. in stirred dry THF 0.1˜0.3 M o PEG was added sodium 0.1˜0.3 eq.) which was cut in small piece under N₂ atmosphere. After sodium disappeared, tert-butyl acrylate (1.0˜1.5 eq.) was added. The mixture was stirred overnight, concentrated in vacuo and purified with silica gel chromatography eluted with EtOAc/DCM (1:10 to 100:1) to afford the title compound (70%˜95% yield).

### Example 295. Synthesis of tert-butyl 2, 5, 8, 11, 14, 17, 20, 23, 26-nonaoxanonacosan-29-oate (262)

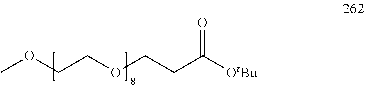

2, 5, 8, 11, 14, 17, 20, 23-octaoxapentacosan-25-ol (25.00 g, 65.06 mmol) in stirred dry THF (450 mL) was added sodium (0.25 g, 10.86 mmol) which was cut in small piece under N₂ atmosphere. After sodium disappeared, tert-butyl acrylate (9.21 g, 71.90 mmol) was added in and the mixture was stirred overnight. The mixture was concentrated in vacuo and purified with silica gel chromatography eluted with EtOAc/DCM (1:5 to 1:2) to afford the title compound (30.97 g, 93% yield). ESI m/z calcd. for C₂₄H₄₉O₁₁ [M+H]⁺: 513.3276, found 512.3298.

### Example 296. Synthesis of tert-butyl 2, 5, 8, 11, 14, 17, 20, 23, 26, 29-decaoxadotriacontan-32-oate (263)

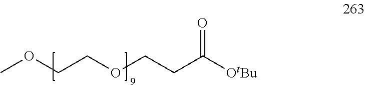

92% yield with 95% purity by HPLC. ESI m/z calcd. for C₂₆H₅₃O₁₂ [M+H]⁺: 557.3538, found 557.3580.

### Example 297. Synthesis of tert-butyl 2, 5, 8, 11, 14, 17, 20, 23-octaoxahexacosan-26-oate (264)

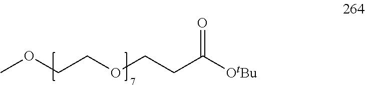

93% yield with 95% purity by HPLC. ESI m/z calcd. for C₂₂H₄₅O₁₀ [M+H]⁺: 469.3013, found 469.3077.

### Example 298. Synthesis of tert-butyl 2, 5, 8, 11, 14, 17, 20-heptaoxatricosan-23-oate (265)

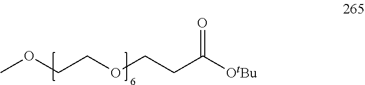

94% yield with 95% purity by HPLC. ESI m/z calcd. for C₂₀H₄₁O₉[M+H]⁺: 425.2771, found 425.2811.

### Example 299. General Synthesis of 3-(ω-Methoxy PEGyl) Propanoic Acid

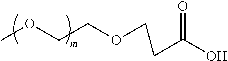

tert-butyl 3-(ω-methoxyl PEGyl)propanoate in dioxane (0.1˜0.3 M) was added concentrated hydrochloride (36%, 1/3 vol of dioxane). The mixture was stirred at r.t. for 30 min, diluted with toluene (1/4˜1/2 vol of dioxane), concentrated in vacuo, co-evaporated with ethanol/toluene (1:1, 2×(1/4˜1/2 vol of original dioxane)) and dried over vacuum pump to afford the title compound (92%-99% yield) which was used for the next step directly. The product was also purified on a short silica gel column eluted with 3%-10% water in CH₃CN or eluted with methanol/DCM (1:8-1:3) containing 1% acetic acid to afford 75%-90% yield with over 95% purity by HPLC.

### Example 300. Synthesis of 2, 5, 8, 11, 14, 17, 20, 23, 26-nonaoxanonacosan-29-oic acid (266)

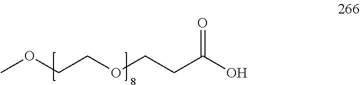

Tert-butyl 2, 5, 8, 11, 14, 17, 20, 23, 26-nonaoxanonacosan-29-oate (10.01 g, 19.53 mmol) in dioxane (75 mL) was added concentrated hydrochloride (25 mL, 36%). The mixture was stirred at r.t. for 30 min, diluted with toluene (50 mL), concentrated in vacuo, co-evaporated with ethanol/toluene (1:1, 2×50 mL) and dried over vacuum pump to afford the title compound (8.55 g, 96% yield) with 95% purity by HPLC. ESI m/z calcd. for C₂₀H₄₁O₁₁ [M+H]⁺: 457.2650, found 457.2683.

### Example 301. Synthesis of 2, 5, 8, 11, 14, 17, 20, 23, 26, 29-decaoxadotriacontan-32-oic acid (267)

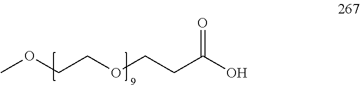

95% yield with 94% purity by HPLC. ESI m/z calcd. for C₂₂H₄₅O₁₂ [M+H]⁺: 501.2912, found 501.2935.

### Example 302. Synthesis of 2, 5, 8, 11, 14, 17, 20, 23-octaoxahexacosan-26-oic acid (268)

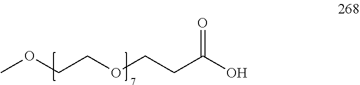

95% yield with 95% purity by HPLC. ESI m/z calcd. for C₁₈H₃₇O₁₀ [M+H]⁺: 413.2387, found 413.2395.

### Example 303. Synthesis of 2, 5, 8, 11, 14, 17, 20-heptaoxatricosan-23-oic acid (269)

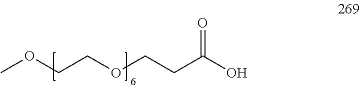

95% yield with 95% purity by HPLC. ESI m/z calcd. for C₁₆H₃₃O₉[M+H]⁺: 369.2125, found 369.2148.

### Example 304. General Synthesis of tert-butyl 3-(PEGyl)propanoate

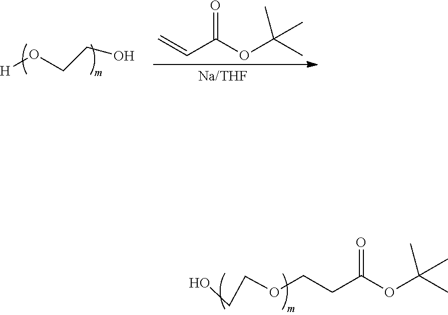

A PEG (1 eq.) in stirred dry THF (0.1˜0.3 M of PEG) was added sodium (0.1˜0.2 eq.) which was cut in small piece under N₂ atmosphere. After sodium disappeared, tert-butyl acrylate (1/4 eq.) was added. The mixture was stirred overnight, concentrated in vacuo and purified with silica gel chromatography eluted with MeOH/DCM (1:8 to 1:4) to afford the title compound (65%˜83% yield).

### Example 305. Synthesis of tert-butyl 1-hydroxy-3, 6, 9, 12, 15, 18, 21, 24, 27-nonaoxatriacontan-30-oate (270)

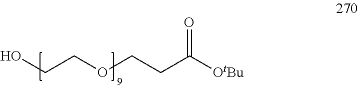

3, 6, 9, 12, 15, 18, 21, 24-octaoxahexacosane-1, 26-diol (40.01 g, 96.58 mmol) in stirred dry THF (600 mL) was added sodium (0.40 g, 17.39 mmol) which was cut in small piece under N₂ atmosphere. After sodium disappeared, tert-butyl acrylate (3.11 g, 24.28 mmol) was added in and the mixture was stirred overnight. The mixture was concentrated in vacuo and purified with silica gel chromatography eluted with MeOH/DCM (1:8 to 1:4) to afford the title compound (10.27 g, 78% yield). ESI m/z calcd. for C₂₅H₅₁O₁₂ [M+H]⁺: 543.3381, found 543.3416.

### Example 306. Synthesis of tert-butyl 1-hydroxy-3, 6, 9, 12, 15, 18, 21, 24-octaoxaheptacosan-27-oate (271)

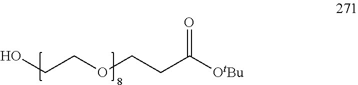

79% yield. ESI m/z calcd. for C₂₃H₄₇O₁₁ [M+H]⁺: 499.3119, found 499.3145.

### Example 307. Synthesis of tert-butyl 1-hydroxy-3, 6, 9, 12, 15, 18, 21-heptaoxatetracosan-24-oate (272)

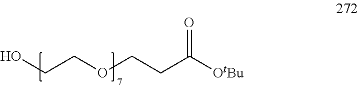

79% yield. ESI m/z calcd. for C₂₁H₄₃O₁₀ [M+H]⁺: 455.2857, found 455.2885.

### Example 308. Synthesis of tert-butyl 1-hydroxy-3, 6, 9, 12, 15, 18-hexaoxahenicosan-21-oate (273)

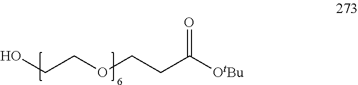

80% yield. ESI m/z calcd. for C₁₉H₃₉O₉[M+H]⁺: 411.2595, found 411.2570.

### Example 309. General Synthesis of tert-butyl 3-(ω-tosyl-PEGyl)propanoate

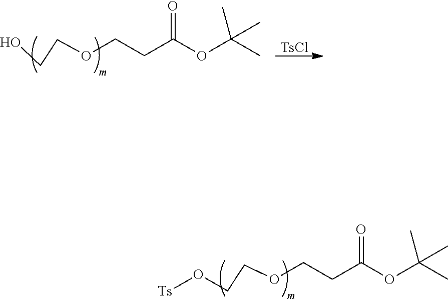

Tert-butyl 3-(PEGyl)propanoate (1 eq.) in the mixture of dry THF/DCM (1:3) and DIPEA (10 eq.) at 4° C. was added tosyl chloride (1.2˜1.5 eq.). Then the mixture was warm to r.t., stirred overnight, concentrated and purified with short silica gel column eluted with MeOH/DCM (1:10-1:8) containing 0.2% acetic acid to afford the title compound (78˜90% yield).

### Example 310. Synthesis of tert-butyl 1-(tosyloxy)-3, 6, 9, 12, 15, 18, 21, 24, 27-nonaoxatriacontan-30-oate (274)

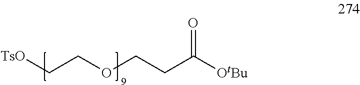

Tert-butyl 1-hydroxy-3, 6, 9, 12, 15, 18, 21, 24, 27-nonaoxatriacontan-30-oate (7.82 g, 14.41 mmol) in the mixture of dry THF/DCM (1:3, 120 mL) and DIPEA (8 mL) at 4° C. was added tosyl chloride (3.57 g, 18.72 mmol). Then the mixture was warm to r.t., stirred overnight, concentrated and purified with short silica gel column eluted with MeOH/DCM (1:10-1:8) to afford the title compound (8.62 g. 86% yield). ESI m/z calcd. for C₃₂H₅₇O₁₄S [M+H]⁺: 697.3480, found 697.3522.

### Example 311. Synthesis of tert-butyl 1-(tosyloxy)-3, 6, 9, 12, 15, 18, 21, 24-octaoxaheptacosan-27-oate (275)

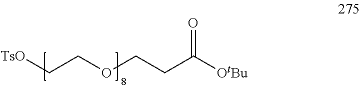

85% yield. ESI m/z calcd. for C₃₀H₅₃O₁₃S [M+H]⁺: 653.3208, found 653.3240.

### Example 312. Synthesis of tert-butyl 1-(tosyloxy)-3, 6, 9, 12, 15, 18, 21-heptaoxatetracosan-24-oate (276)

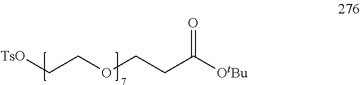

86% yield. ESI m/z calcd. for C₂₈H₄₉O₁₂S [M+H]⁺: 609.2945, found 609.2968.

### Example 313 Synthesis of tert-butyl 1-(tosyloxy)-3, 6, 9, 12, 15, 18-hexaoxahenicosan-21-oate (277)

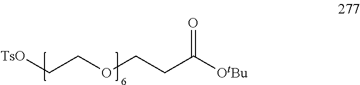

87% yield. ESI m/z calcd. for C₂₆H₄₅O₁₁S [M+H]: 565.2683, found 565.2705.

### Example 314. General Synthesis of tert-butyl 3-(ω-azido-PEGyl)propanoate

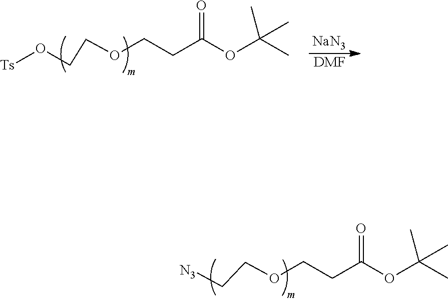

NaN₃ (1.5˜3 eq.) stirred in DMF (60 mL) was added tert-butyl 3-(ω-tosyloxy-PEGyl)propanoate (1 eq.). The mixture was stirred overnight, concentrated and purified with short silica gel column eluted with MeOH/DCM (1:15-1:8) to afford the title compound (83%˜91% yield).

### Example 315. Synthesis of tert-butyl 1-azido-3, 6, 9, 12, 15, 18, 21, 24, 27-nonaoxatriacontan-30-oate (278)

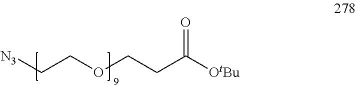

NaN₃ (2.00 g, 30.76 mmol) stirred in DMF (60 mL) was added tert-butyl 1-(tosyloxy)-3, 6, 9, 12, 15, 18, 21, 24, 27-nonaoxatriacontan-30-oate (7.51 g, 10.78 mmol). The mixture was stirred overnight, concentrated and purified with short silica gel column eluted with MeOH/DCM (1:15-1:10) to afford the title compound (5.32 g. 84% yield). ESI m/z calcd. for C₂₅H₅₀N₃O₁₁ [M+H]⁺: 568.3446, found 568.3467.

### Example 316. Synthesis of tert-butyl 1-azido-3, 6, 9, 12, 15, 18, 21, 24-octaoxaheptacosan-27-oate (279)

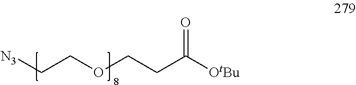

84% yield. ESI m/z calcd. for C₂₃H₄₆N₃O₁₀ [M+H]⁺: 524.3184, found 524.3205.

### Example 317. Synthesis of tert-butyl 1-azido-3, 6, 9, 12, 15, 18, 21-heptaoxatetracosan-24-oate (280)

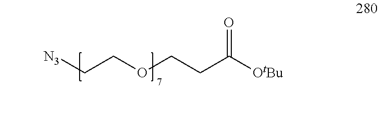

85% yield. ESI m/z calcd. for C₂₁H₄₂N₃O₉ [M+H]⁺: 480.2922, found 480.2945.

### Example 318. Synthesis of tert-butyl 1-azido-3, 6, 9, 12, 15, 18-hexaoxahenicosan-21-oate (281)

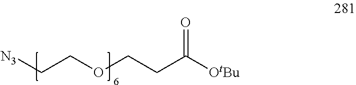

85% yield. ESI m/z calcd. for C₁₉H₃₈N₃O₈ [M+H]⁺: 436.2660, found 436.2695.

### Example 319. General Synthesis of tert-butyl 3-(ω-amino-PEGyl)propanoate

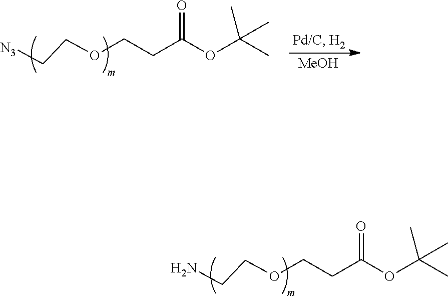

Tert-3-(ω-azido-PEGyl)propanoate (1 eq.) in methanol (0.15˜0.2 M conc.) in a hydrogenation bottle was added Pd/C (10% Pd, 2%˜10% by weight of the starting material). Then the mixture was conducted with H₂ at 5˜50 psi, shaken 2˜12 h, filtrated through Celite, concentrated and dried over vacuum to afford the title compound (87˜95% yield), which was used for the next step without further purification.

### Example 320. Synthesis of tert-butyl 1-amino-3, 6, 9, 12, 15, 18, 21, 24, 27-nonaoxatriacontan-30-oate (282)

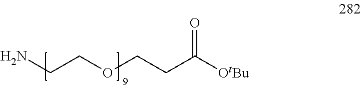

tert-butyl 1-azido-3, 6, 9, 12, 15, 18, 21, 24, 27-nonaoxatriacontan-30-oate (3.22 g, 5.67 mmol) was added in metanol (80 mL) containing Pd/C (0.20 g, 10% Pd). The mixture was conducted with hydrogen (25 psi), shaken 6 h, filtrated through Celite, concentrated and dried over vacuum to afford the title compound (90% yield), which was used for the next step without further purification. ESI m/z calcd. for C₂₅H₅₂NO₁₁ [M+H]⁺: 542.3541, found 542.3575.

### Example 321. Synthesis of tert-butyl 1-amino-3, 6, 9, 12, 15, 18, 21, 24-octaoxaheptacosan-27-oate (283)

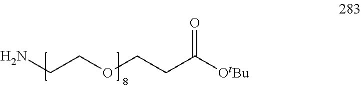

>90% yield. ESI m/z calcd. for C₂₃H₄₈NO₁₀ [M+H]⁺: 498.3279, found 498.3315.

### Example 322. Synthesis of tert-butyl 1-amino-3, 6, 9, 12, 15, 18, 21-heptaoxatetracosan-24-oate (284)

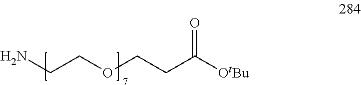

>90% yield. ESI m/z calcd. for C₂₁H₄₄NO₉ [M+H]⁺: 454.3017, found 454.3035.

### Example 323. Synthesis of tert-butyl 1-amino-3, 6, 9, 12, 15, 18-hexaoxahenicosan-21-oate (285)

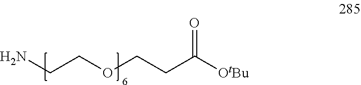

>90% yield. ESI m/z calcd. for C₁₉H₄₀NO₈ [M+H]⁺: 410.2755, found 410.2780.

### Example 324. General Synthesis of tert-butyl 3-(ω-(3′-(ω′-methoxy PEGyl)-propanamido)-PEGyl)propanoate

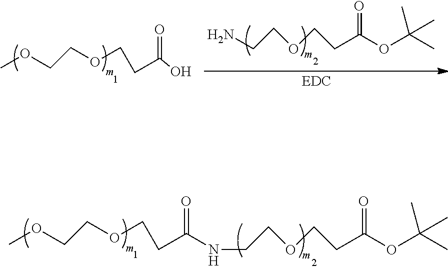

Tert-butyl 3-(ω-amino-PEGyl)propanoate (1 eq.) and 3-(ω-methoxy PEGyl) propanoic acid (1 eq.) in DMF (0.1-3.0 M conc. of the starting material) was added EDC (1.2˜3.0 eq.). The mixture was stirred overnight, concentrated in vacuo and purified with silica gel chromatography eluted with MeOH/DCM (1:8 to 1:2) to afford the title compound (63%˜88% yield).

### Example 325. Synthesis of tert-butyl 23-oxo-2, 5, 8, 11, 14, 17, 20, 27, 30, 33, 36, 39, 42-tridecaoxa-24-azapentatetracontan-45-oate (286)

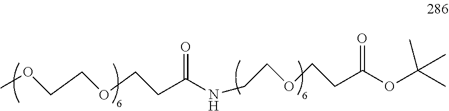

2, 5, 8, 11, 14, 17, 20-Heptaoxatricosan-23-oic acid (5.011 g, 13.60) and tert-butyl 1-amino-3, 6, 9, 12, 15, 18-hexaoxahenicosan-21-oate (5.57 g, 13.60 mmol) in DMF (75 mL) was added EDC (5.25 g, 27.34 mmol). The mixture was stirred for 6 h, concentrated in vacuo and purified with silica gel chromatography eluted with MeOH/DCM (1:8 to 1:5) to afford the title compound (8.882 g, 86% yield). ESI m/z calcd. for C₃₅H₇₀NO₁₆ [M+H]⁺: 760.4695, found 760.4735.

### Example 326. General Synthesis of 3-(ω-(3′-(ω′-methoxy PEGyl)-propanamido)-PEGyl) propanoic acid

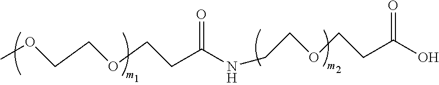

tert-butyl 3-(ω-(3′-(ω′-methoxy PEGyl)-propanamido)-PEGyl)propanoate in dioxane (0.2-1.0 M conc. of the starting material) was added HCl (conc. 25% v/v of dioxane). The mixture was stirred for 0.5 h, diluted with toluene, concentrated in vacuo to afford the title compound (90-102% yield).

### Example 327. Synthesis of 23-oxo-2, 5, 8, 11, 14, 17, 20, 27, 30, 33, 36, 39, 42-tridecaoxa-24-azapentatetracontan-45-oic acid (287)

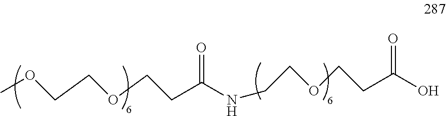

Tert-butyl 23-oxo-2, 5, 8, 11, 14, 17, 20, 27, 30, 33, 36, 39, 42-tridecaoxa-24-azapentatetracontan-45-oate (5.25 g, 6.91 mmol) in dioxane (20 mL) was added HCl (conc., 5 mL). The mixture was stirred for 0.5 h, diluted with toluene, concentrated in vacuo to afford the title compound (4.85 g, 99% yield). ESI m/z calcd. for C₃₁H₆₂NO₁₆ [M+H]⁺: 704.4069, found 704.4105.

### Example 328. Synthesis of ethyl 2-((R, E)-3-(((S)-tert-butylsulfinyl)imino)-1-hydroxy-4-methylpentyl)thiazole-4-carboxylate (288)

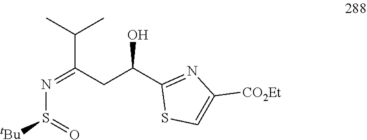 [SMILES: CCOC(=O)c1csc([C@H](O)C/C(=N\[S@@](=O)C(C)(C)C)C(C)C)n1]

To a solution of diisopropylamine (121 mL, 0.86 mol, 4.0 eq.) in dry THF (300 mL) was added n-butyllithium (2.5 M, 302 mL, 0.76 mol 3.5 eq.) at −78° C. under N₂. The reaction mixture was warmed to 0° C. over 30 min and then cooled back to −78°. (S, E)-2-methyl-N—(3-methylbutan-2-ylidene)propane-2-sulfonamide (57 g, 0.3 mol, 1.4 eq.) in THF (200 mL) was added. The reaction mixture was stirred for 1 h before ClTi(OⁱPr)₃ (168.5 g, 0.645 mol, 3.0 eq.) in THF (350 mL) was added dropwise. After stirring for 1 h, ethyl 2-formylthiazole-4-carboxylate (40 g, 0.215 mol, 1.0 eq.) dissolved in THF (175 mL) was added dropwise and the resulting reaction mixture was stirred for 2 h. The completion of the reaction was indicated by TLC analysis. The reaction was quenched by a mixture of acetic acid and THF (v/v 1:4, 200 mL), then poured onto iced water, extracted with ethyl acetate (4×500 mL). The organic phase was washed with water and brine, dried over anhydrous sodium sulfate, filtered and concentrated. The residue was purified by column chromatography (DCM/EtOAc/PE 2:1:2) to afforded the title compound (60 g, 74% yield) as a colorless oil. ¹H NMR (500 MHz, CDCl₃) δ 8.13 (s, 1H), 6.63 (d, J=8.2 Hz, 1H), 5.20-5.11 (m, 1H), 4.43 (q, J=7.0 Hz, 2H), 3.42-3.28 (m, 2H), 2.89 (dt, J=13.1, 6.5 Hz, 1H), 1.42 (t, J=7.1 Hz, 3H), 1.33 (s, 9H), 1.25-1.22 (m, 6H). ESI MS m/z calcd. for C₁₆H₂₆NaN₂04S₂ [M+Na]⁺397.13, found 397.11.

### Example 329. Synthesis of ethyl 2-((1R, 3R)-3-((S)-1, 1-dimethylethylsulfinamido)-1-hydroxy-4-methylpentyl)thiazole-4-carboxylate (289)

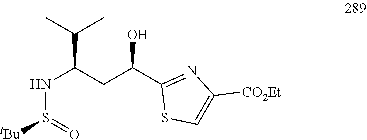 [SMILES: CCOC(=O)c1csc([C@H](O)C[C@@H](N[S@@](=O)C(C)(C)C)C(C)C)n1]

A solution of ethyl 2-((R, E)-3-(((S)-tert-butylsulfinyl)imino)-1-hydroxy-4-methylpentyl) thiazole-4-carboxylate (23.5 g, 62.7 mmol) dissolved in THF (200 mL) was cooled to −45° C. Ti(OEt)₄ (42.9 mL, 188 mmol, 3.0 eq.) was added slowly. After the completion of addition, the mixture was stirred for 1 h, before NaBH₄ (4.75 g, 126 mmol, 2.0 eq.) was added in portions. The reaction mixture was stirred at −45° C. for 3 h. TLC analysis showed some starting material still remained. The reaction was quenched with HOAc/THF (v/v 1:4, 25 mL), followed by EtOH (25 mL). The reaction mixture was poured onto ice (100 g) and warmed to r.t. After filtration over Celite, the organic phase was separated and washed with water and brine, dried over anhydrous sodium sulfate, filtered, and concentrated. The residue was purified by column chromatography (EtOAc/PE 1:1) to deliver the title product (16.7 g, 71% yield) as a white solid. ¹H NMR (500 MHz, CDCl₃) δ 8.10 (s, 1H), 5.51 (d, J=5.8 Hz, 1H), 5.23-5.15 (m, 1H), 4.41 (q, J=7.0 Hz, 2H), 3.48-3.40 (m, 1H), 3.37 (d, J=8.3 Hz, 1H), 2.29 (t, J=13.0 Hz, 1H), 1.95-1.87 (m, 1H), 1.73-1.67 (m, 1H), 1.40 (t, J=7.1 Hz, 3H), 1.29 (s, 9H), 0.93 (d, J=7.3 Hz, 3H), 0.90 (d, J=7.2 Hz, 3H). ESI MS m/z calcd. for C₁₆H₂₈NaN₂O₄S₂ [M+Na]⁺399.15, found 399.14.

### Example 330. Synthesis of ethyl 2-((1R, 3R)-3-amino-I-hydroxy-4-methylpentyl) thiazole-4-carboxylate hydrochloride (290)

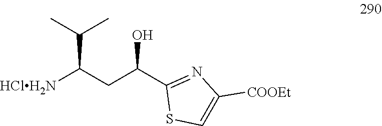

To a solution of ethyl 2-((1R, 3R)-3-((S)-1, 1-dimethylethylsulfinamido)-1-hydroxy-4-methylpentyl)thiazole-4-carboxylate (6.00 g, 16.0 mmol, 1.0 eq.) in ethanol (40 mL) was added 4 N HCl in dioxane (40 mL) slowly at 0° C. The reaction was allowed to warm to r.t. and stirred for 2.5 h then concentrated and triturated with petroleum ether. A white solid title compound (4.54 g, 92% yield) was collected and used in the next step.

### Example 331. Synthesis of ethyl 2-((1R, 3R)-3-((2S, 3S)-2-azido-3-methylpentanamido)-1-hydroxy-4-methylpentyl)thiazole-4-carboxylate (291)

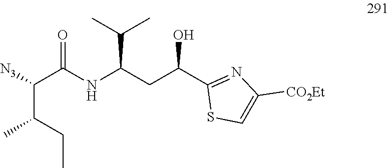 [SMILES: CCOC(=O)c1csc([C@H](O)C[C@@H](NC(=O)[C@@H](N=[N+]=[N-])[C@@H](C)CC)C(C)C)n1]

(2S, 3S)-2-azido-3-methylpentanoic (5.03 g, 28.8 mmol, 2.0 eq.) was dissolved in THF (120 mL) and cooled to 0° C., to which NMM (6.2 mL, 56.0 mmol, 4.0 eq.) and isobutylchloroformate (3.7 mL, 28.8 mmol, 2.0 eq.) were added in sequence. The reaction was stirred at 0° C. for 30 min and r.t. 1.0 h, and then cooled back to 0° C. Ethyl 2-((1R, 3R)-3-amino-1-hydroxy-4-methylpentyl)thiazole-4-carboxylate hydrochloride (4.54 g, 14.7 mmol, 1.0 eq.) was added in portions. After stirring at 0° C. for 30 min, the reaction was warmed to r.t. and stirred for 2 h. Water was added at 0° C. to quenched the reaction and the resulting mixture was extracted with ethyl acetate for three times. The combined organic layers were washed with 1N HCl, saturated NaHCO₃ and brine, dried over anhydrous sodium sulfate, filtered and concentrated. The residue was purified by column chromatography (0-30% EtOAc/PE) to give a white solid title compound (4.55 g, 74% yield).

### Example 332. Synthesis of ethyl 2-((1R, 3R)-3-((2S, 3S)-2-azido-3-methylpentanamido)-4-methyl-1-((triethylsilyl)oxy)pentyl)thiazole-4-carboxylate (292)

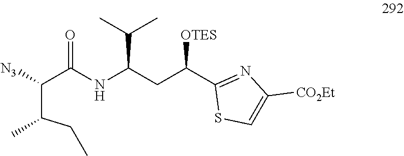

To a solution of ethyl 2-((1R, 3R)-3-((2S, 3S)-2-azido-3-methylpentanamido)-1-hydroxy-4-methylpentyl)thiazole-4-carboxylate (5.30 g, 12.8 mmol, 1.0 eq.) in dichloromethane (50 mL) was added imidazole (1.75 g, 25.6 mmol, 2.0 eq.), followed by chlorotriethylsilane (4.3 mL, 25.6 mmol, 2.0 eq.) at 0° C. The reaction mixture was allowed to warm to r.t. over 1 hour and stirred for an additional hour. Brine was added to the reaction mixture, the organic layer was separated and the aqueous layer was extracted with EtOAc. The combined organic phases were dried, filtered, concentrated under reduced pressure, and purified by column chromatography with a gradient of 15-35% ethyl acetate in petroleum ether to afford the title product (6.70 g, 99% yield) as a white solid. ¹H NMR (500 MHz, CDCl₃) δ 8.12 (s, 1H), 6.75 (d, J=8.0 Hz, 1H), 5.20-5.12 (m, 1H), 4.44 (q, J=7.0 Hz, 2H), 4.06-3.97 (m, 1H), 3.87 (d, J=3.8 Hz, 1H), 2.14 (d, J=3.8 Hz, 1H), 2.01-1.91 (m, 3H), 1.42 (t, J=7.1 Hz, 3H), 1.34-1.25 (m, 2H), 1.06 (d, J=6.8 Hz, 3H), 1.00-0.93 (m, 18H), 0.88 (dd, J=19.1, 6.8 Hz, 6H). ESI MS m/z calcd. for C₂₄H₄₄N₅O₄SSi [M+H]⁺ 526.28, found 526.28.

### Example 333. Synthesis of ethyl 2-((1R, 3R)-3-((2S, 3S)-2-azido-N, 3-dimethyl pentanamido)-4-methyl-1-((triethylsilyl)oxy)pentyl)thiazole-4-carboxylate (293)

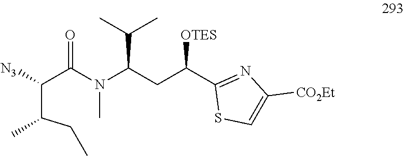

A solution of ethyl 2-((1R, 3R)-3-((2S, 3S)-2-azido-3-methylpentanamido)-4-methyl-1-((triethylsilyl)oxy)pentyl)thiazole-4-carboxylate (5.20 g, 9.9 mmol, 1.0 eq.) in THF (50 mL) was cooled to −45° C. and KHMDS (1M in toluene, 23.8 mL, 23.8 mmol, 2.4 eq.) was added. The resulting mixture was stirred at −45° C. for 20 min, followed by addition of methyl iodide (1.85 mL, 29.7 mmol, 3.0 eq.). The reaction mixture was warmed to r.t. over 4.5 h, then the reaction was quenched with EtOH (10 mL). The crude product was diluted with EtOAc (250 mL) and washed with brine (100 mL). The aqueous layer was extracted with ethyl acetate (3×50 mL). The organic layers were dried, filtered, concentrated and purified on column chromatography with a gradient of 15-35% ethyl acetate in petroleum ether to afford the title product (3.33 g, 63% yield) as a light yellow oil. ¹H NMR (500 MHz, CDCl₃) δ 8.09 (s, 1H), 4.95 (d, J=6.6 Hz, 1H), 4.41 (q, J=7.1 Hz, 2H), 3.56 (d, J=9.5 Hz, 1H), 2.98 (s, 3H), 2.27-2.06 (m, 4H), 1.83-1.70 (m, 2H), 1.41 (t, J=7.2 Hz, 3H), 1.29 (ddd, J=8.9, 6.8, 1.6 Hz, 3H), 1.01 (d, J=6.6 Hz, 3H), 0.96 (dt, J=8.0, 2.9 Hz, 15H), 0.92 (d, J=6.6 Hz, 3H), 0.90 (d, J=6.7 Hz, 3H). ESI MS m/z calcd. for C₂₅H₄₆N₅O₄SSi [M+H]⁺ 540.30, found 540.30.

### Example 334. Synthesis of ethyl 2-((6S, 9R, 11R)-6-((S)-sec-butyl)-13, 13-diethyl-9-isopropyl-2, 3, 3, 8-tetramethyl-4, 7-dioxo-12-oxa-2, 5, 8-triaza-13-silapentadecan-11-yl)thiazole-4-carboxylate (294)

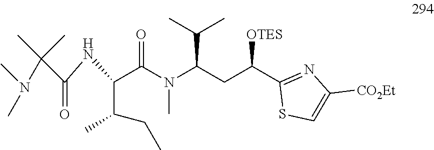

Dry Pd/C (10 wt %, 300 mg) and ethyl 2-((1R, 3R)-3-((2S, 3S)-2-azido-N, 3-dimethyl pentanamido)-4-methyl-1-((triethylsilyl)oxy)pentyl)thiazole-4-carboxylate (3.33 g, 6.16 mmol) were added to perfluorophenyl 2-(dimethylamino)-2-methylpropanoate (˜2.75 g, 1.5 eq.) in ethyl acetate. The reaction mixture was stirred under hydrogen atmosphere for 27 h, and then filtered through a plug of Celite, with washing of the filter pad with ethyl acetate. The combined organic portions were concentrated and purified by column chromatography with a gradient of 0-5% methanol in ethyl acetate to deliver the title product (3.24 g, 84% yield). ESI MS m/z calcd. for C₃₁H₅₉N₄O₅SSi [M+H]⁺ 626.39, found 626.95.

### Example 335. Synthesis of ethyl 2-((1R, 3R)-3-((2S, 3S)-2-(2-(dimethylamino)-2-methylpropanamido)-N, 3-dimethylpentanamido)-1-hydroxy-4-methylpentyl)thiazole-4-carboxylate (295)

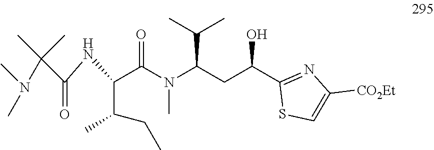 [SMILES: CCOC(=O)c1csc([C@H](O)C[C@H](C(C)C)N(C)C(=O)[C@@H](NC(=O)C(C)(C)N(C)C)[C@@H](C)CC)n1]

Ethyl 2-((6S, 9R, 11R)-6-((S)-sec-butyl)-13, 13-diethyl-9-isopropyl-2, 3, 3, 8-tetramethyl-4, 7-dioxo-12-oxa-2, 5, 8-triaza-13-silapentadecan-11-yl)thiazole-4-carboxylate (3.20 g, 5.11 mmol) was dissolved in deoxygenated AcOH/water/THF (v/v/v 3:1:1, 100 mL), and stirred at r.t. for 48 h. The reaction was then concentrated and purified on silica gel column chromatography (2:98 to 15:85 MeOH/EtOAc) to afford the title compound (2.33 g, 89% yield). ESI MS m/z calcd. for C₂₅H₄₅N₄O₅S [M+H]⁺ 512.30, found 512.45.

### Example 336. Synthesis of 2-((1R, 3R)-3-((2S, 3S)-2-(2-(dimethylamino)-2-methylpropanamido)-N, 3-dimethylpentanamido)-1-hydroxy-4-methylpentyl)thiazole-4-carboxylic acid (296)

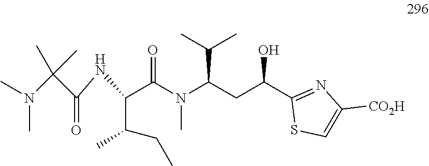 [SMILES: CC[C@H](C)[C@H](NC(=O)C(C)(C)N(C)C)C(=O)N(C)[C@H](C[C@@H](O)c1nc(C(=O)O)cs1)C(C)C]

An aqueous solution of LiOH (0.4 N, 47.7 mL, 19.1 mmol, 4.0 eq.) was added to a solution of ethyl 2-((1R, 3R)-3-((2S, 3S)-2-(2-(dimethylamino)-2-methylpropanamido)-N, 3-dimethylpentanamido)-1-hydroxy-4-methylpentyl)thiazole-4-carboxylate (2.30 g, 4.50 mmol, 1.0 eq.) in methanol (50 mL) at 0° C. The reaction mixture was stirred at r.t. for 2 h and then concentrated. Silica gel column chromatographic purification (100% dichloromethane then DCM/MeOH/NH₄OH 80:20:1) afforded the title compound (2.13 g, 98% yield) as an amorphous solid. ESI MS m/z calcd. for C₂₃H₄₁N₄O₅S [M+H]⁺ 485.27, found 485.55.

### Example 337. Synthesis of 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylic acid (297)

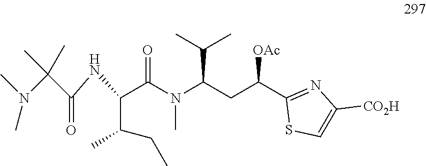 [SMILES: CC[C@H](C)[C@H](NC(=O)C(C)(C)N(C)C)C(=O)N(C)[C@H](C[C@@H](OC(C)=O)c1nc(C(=O)O)cs1)C(C)C]

To a solution of 2-((1R, 3R)-3-((2S, 3S)-2-(2-(dimethylamino)-2-methylpropan amido)-N, 3-dimethylpentanamido)-1-hydroxy-4-methylpentyl)thiazole-4-carboxylic acid (2.10 g, 4.33 mmol) in pyridine (50 mL) at 0° C., acetic anhydride (2.25 mL, 24 mmol) was added slowly. The reaction mixture was warmed to r.t. over 2 h and stirred at r.t. for 24 h. The reaction was concentrated and the residue was purified on reverse phase HPLC (C₁₈ column, 50 mm×250 (mm), 50 mL/min, 10-90% acetonitrile/water in 45 min) to afford the title compound (1.95 g, 86% yield) as an amorphous white solid. ESI MS m/z calcd. for C₂₅H₄₃N₄O₆S [M+H]⁺ 526.28, found 526.80.

### Example 338. Synthesis of ethyl 2-((1R, 3R)-3-((2S, 3S)-2-azido-N, 3-dimethylpentanamido)-1-methoxy-4-methylpentyl)thiazole-4-carboxylate (298)

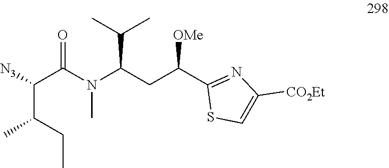 [SMILES: CCOC(=O)c1csc([C@@H](C[C@H](C(C)C)N(C)C(=O)[C@@H](N=[N+]=[N-])[C@@H](C)CC)OC)n1]

Compound 291 (130 g, 0.30 mol) was dissolved in dry tetrahydrofuran (1.6 L), to which methyl iodide (255 g, 1.80 mol) was added over an ice bath, followed by NaH (60 g, 60 wt %, 0.45 mol) in three portions. The reaction was warmed to room temperature naturally, and stirred overnight. HPLC-MS analysis indicated that the starting materials was completely consumed, with a little monomethyl substituted by-product. The reaction solution was poured into 2 L ice-cooled saturated NH₄C₁, extracted with ethyl acetate (2 L, 1 L). The organic phase was washed twice with water, once with brine, dried with anhydrous sodium sulfate, and concentrated to give a crude product. The crude product was triturated with PE to afford 108 g of white solid (yield 78%).

### Example 339. Synthesis of ethyl 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylate (299)

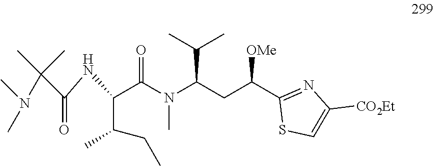 [SMILES: CCOC(=O)c1csc([C@@H](C[C@H](C(C)C)N(C)C(=O)[C@@H](NC(=O)C(C)(C)N(C)C)[C@@H](C)CC)OC)n1]

To a solution of compound 298 (100 g, 0.23 mol) in 200 mL of ethyl acetate were added perfluorophenyl 2-(dimethylamino)-2-methylpropanoate (0.57 mol in 1000 mL of ethyl acetate) and palladium carbon (10 g, 5 wt %, 50% wet). The mixture was stirred under H₂ balloon, and after exchanging the nitrogen for several times, the reaction was stirred overnight. HPLC-MS indicated that the complete consumption of the starting material. The reaction solution was filtered, and the filter cake was washed with ethyl acetate, the combined filtrate was collected, concentrated, and purified by column chromatography (20%-60% EtOAc/PE) to give 100 g of the title product (84% yield).

### Example 340. Synthesis of 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylic acid (300)

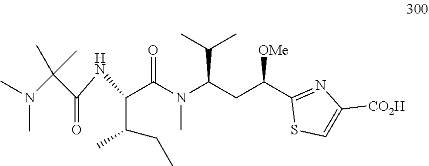 [SMILES: CC[C@H](C)[C@H](NC(=O)C(C)(C)N(C)C)C(=O)N(C)[C@H](C[C@@H](OC)c1nc(C(=O)O)cs1)C(C)C]

To a solution of compound 299 (121 g, 0.23 mol) in 1, 4-dioxane (1000 mL) and water (100 mL), LiOH (22 g, 0.92 mol) dissolved in water (300 mL) was slowly added to the reaction system. After stirring at room temperature for 2 h, HPLC-MS indicated completion of the reaction. The reaction was concentrated and mixed with 200 g silica gel, loaded on column, and eluted with 50%-100% EtOAc/PE and 0%-20% MeOH/DCM to give the title compound (94 g, 84% yield).

### Example 341. Synthesis of (S, Z)-tert-butyl 5-(4-(benzyloxy)phenyl)-4-((tert-but oxycarbonyl)amino)-2-methylpent-2-enoate (301)

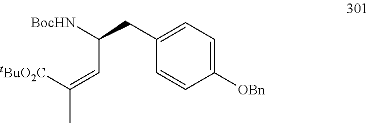 [SMILES: C/C(=C/[C@H](Cc1ccc(OCc2ccccc2)cc1)NC(=O)OC(C)(C)C)C(=O)OC(C)(C)C]

(S)-tert-Butyl (1-(4-(benzyloxy)phenyl)-3-oxopropan-2-yl)carbamate (0.84 g, 2 mmol, 1.0 eq.) was dissolved in dry dichloromethane (50 mL), to which tert-butyl 2-(triphenyl-phosphoranylidene)propanoate (1.6 g, 4 mmol, 2.0 eq.) was added and the solution was stirred at r.t. for 1.5 h as determined complete by TLC. Purification by column chromatography (10-50% EtOAc/hexanes) afforded the title compound (1.16 g, 98% yield).

### Example 342. Synthesis of (4R)-tert-butyl 4-((tert-butoxycarbonyl)amino)-5-(4-hydroxyphenyl)-2-methylpentanoate (302)

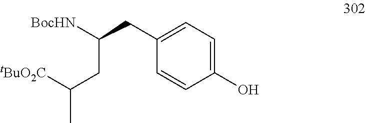 [SMILES: CC(C[C@H](Cc1ccc(O)cc1)NC(=O)OC(C)(C)C)C(=O)OC(C)(C)C]

(S, Z)-tert-Butyl 5-(4-(benzyloxy)phenyl)-4-((tert-but oxycarbonyl)amino)-2-methylpent-2-enoate (467 mg, 1 mmol) was dissolved in methanol (30 mL) and hydrogenated (1 atm H₂) with Pd/C catalyst (10 wt %, 250 mg) at r.t. overnight. The catalyst was filtered off and the filtrate were concentrated under reduced pressure to afford the title compound (379 mg, 99% yield).

### Example 343. Synthesis of (4R)-tert-butyl 4-((tert-butoxycarbonyl)amino)-5-(4-hydroxy-3-nitrophenyl)-2-methylpentanoate (303)

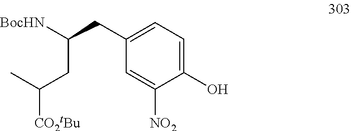 [SMILES: CC(C[C@H](Cc1ccc(O)c([N+](=O)[O-])c1)NC(=O)OC(C)(C)C)C(=O)OC(C)(C)C]

(4R)-tert-Butyl 4-((tert-butoxycarbonyl)amino)-5-(4-hydroxyphenyl)-2-methyl pentanoate (379 mg, 1 mmol, 1.0 eq.) was dissolved in THF (20 mL), to which a solution of tert-butyl nitrite (315 mg, 3 mmol, 3.0 eq.) in THF (2 mL) was added. The reaction was stirred at r.t. for 3 h and then poured onto water, extracted with ethyl acetate (2×50 mL) and the combined organic phases were washed with brine (50 mL), dried over anhydrous sodium sulfate, filtered and concentrated. Purification by column chromatography (10-50% EtOAc/hexanes) afforded the title compound (300 mg, 71% yield).

### Example 344. Synthesis of (4R)-tert-butyl 5-(3-amino-4-hydroxyphenyl)-4-((tert-butoxycarbonyl)amino)-2-methylpentanoate (304)

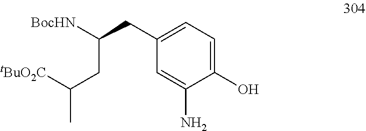 [SMILES: CC(C[C@H](Cc1ccc(O)c(N)c1)NC(=O)OC(C)(C)C)C(=O)OC(C)(C)C]

(4R)-Tert-butyl 4-((tert-butoxycarbonyl)amino)-5-(4-hydroxy-3-nitrophenyl)-2-methyl-pentanoate (200 mg, 0.47 mmol) was dissolved in ethyl acetate (30 mL) and mixed with palladium catalyst (10% on carbon, 100 mg), then hydrogenated (1 atm) at r.t. for 2 h. The catalyst was filtered off and all volatiles were removed under vacuum, which afforded the title compound (185 mg, 99%). ESI MS m/z calcd. for C₂₁H₃₅N₂O₅[M+H]⁺ 395.25, found 395.26.

### Example 345. Synthesis of (4R)-tert-butyl 5-(3-(4-(((benzyloxy)carbonyl)amino) butanamido)-4-hydroxyphenyl)-4-((tert-butoxycarbonyl)amino)-2-methylpentanoate (305)

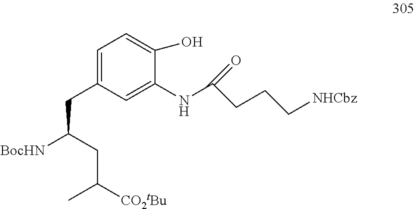 [SMILES: CC(C[C@H](Cc1ccc(O)c(NC(=O)CCCNC(=O)OCc2ccccc2)c1)NC(=O)OC(C)(C)C)C(=O)OC(C)(C)C]

HATU (39.9 g, 105 mmol) was added to a solution of 4-(((benzyloxy)carbonyl) amino) butanoic acid (26.1 g, 110 mmol) in DMF (300 mL). After stirring at r.t. for 30 min, the mixture was added to a solution of compound 304 (39.4 g, 100 mmol) and TEA (20.2 g, 200 mmol) in DMF (300 mL). The resulting mixture was stirred at r.t. for 2 h. Water was then added, extracted with ethyl acetate, the organic layer was washed with brine, dried over sodium sulfate. Purification by column chromatography (20%-70% EtOAc/PE) yielded the title product as a white solid (45 g, 73% yield). ESI m/z calcd. for C₃₃H₄₈N₃O₈ [M+H]⁺: 614.34, found 614.15.

### Example 346. Synthesis of (4R)-tert-butyl 5-(3-(4-aminobutanamido)-4-hydroxyphenyl)-4-((tert-butoxycarbonyl)amino)-2-methylpentanoate (306)

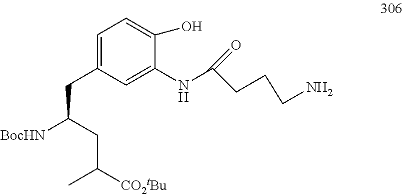 [SMILES: CC(C[C@H](Cc1ccc(O)c(NC(=O)CCCN)c1)NC(=O)OC(C)(C)C)C(=O)OC(C)(C)C]

Compound 305 (100 g, 163 mmol) was dissolved in methanol (500 mL) and hydrogenated (1 atm H₂) with Pd/C catalyst (10 wt %, 10 g) at r.t. overnight. The catalyst was filtered off and the filtrate were concentrated under reduced pressure to afford the title compound (75.8 g, 97% yield) as a brown foamy solid. ¹H NMR (400 MHz, CDCl3) δ 7.11 (s, 1H), 6.83 (d, J=10.3 Hz, 2H), 5.04-4.52 (m, 6H), 3.90-3.56 (m, 1H), 2.81 (d, J=5.3 Hz, 2H), 2.63 (dd, J=12.5, 6.1 Hz, 2H), 2.54-2.26 (dd, J=14.0, 7.6 Hz, 4H), 1.94-1.64 (m, 3H), 1.44-1.36 (m, 18H), 1.08 (d, J=6.9 Hz, 3H). ESI m/z calcd. for C₂₅H₄₂N₃O₆ [M+H]⁺: 480.30, found 480.59.

### Example 347. Synthesis of Compound (4R)-tert-butyl 5-(3-((S)-37-(((benzyloxy)carbonyl) amino)-31, 38-dioxo-2, 5, 8, 11, 14, 17, 20, 23, 26, 29-decaoxa-32, 39-diazatritetracontanamido)-4-hydroxyphenyl)-4-((tert-butoxycarbonyl)amino)-2-methylpentanoate (307)

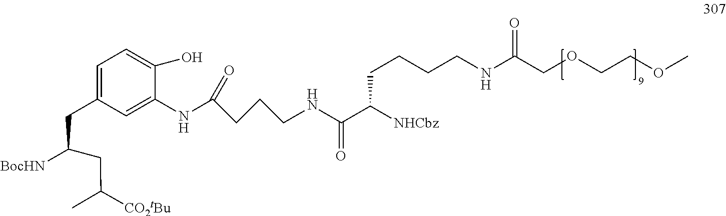

To a solution of (S)-37-(((benzyloxy)carbonyl)amino)-31-oxo-2,5,8,11,14,17,20,23,26,29-decaoxa-32-azaoctatriacontan-38-oic acid (130 g, 174 mmol, 1.1 eq.) in DMF (500 mL) were added TEA (66 mL, 474 mmol, 3eq.) and HATU (72 g, 190 mmol, 1.2 eq.) in sequence at 0° C. Then the reaction mixture was warmed to r.t and stirred for 2 h. A solution of compound 306 (75.8 g, 158 mmol, 1.0 eq.) in DMF (500 mL) was added to the above solution at 0° C., and the reaction mixture was stirred at r.t. for 1 h. The reaction mixture was poured into water (4 L), the aqueous layer was extracted with ethyl acetate (3×500 mL), and the organic layers were combined and washed with brine (2 L), dried over sodium sulfate, concentrated and the crude title product (190 g) was used in the next step directly. ESI MS m/z: calcd. for C₆₀H₁₀₀N₅O₂₀[M+H]⁺: 1210.69, found 1210.69.

### Example 348. Synthesis of (4R)-tert-butyl 5-(3-((S)-37-amino-31, 38-dioxo-2, 5, 8, 11, 14, 17, 20, 23, 26, 29-decaoxa-32, 39-diazatritetracontanamido)-4-hydroxyphenyl)-4-((tert-butoxycarbonyl)amino)-2-methylpentanoate (308)

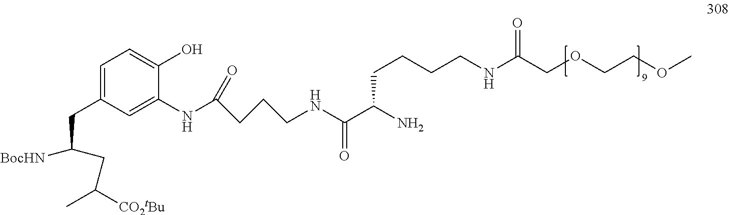

The crude product from previous reaction 307 (190 g) was dissolved in methanol (900 mL) and hydrogenated (1 atm H₂) with Pd/C catalyst (10 wt %, 19 g) at r.t. overnight. The catalyst was filtered off and the filtrate were concentrated under reduced pressure, and the crude compound was purified by silica gel column with a gradient of DCM/MeOH to give the title product (105 g, 62% yield over two steps) as a brown oil. ESI MS m/z calcd. for C₅₂H₉₅N₅O₁₈ [M+H]⁺: 1077.65, found 1077.65.

### Example 349. Synthesis of (4R)-tert-butyl 4-((tert-butoxycarbonyl)amino)-5-(3-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontanamido)-4-hydroxyphenyl)-2-methylpentanoate (309)

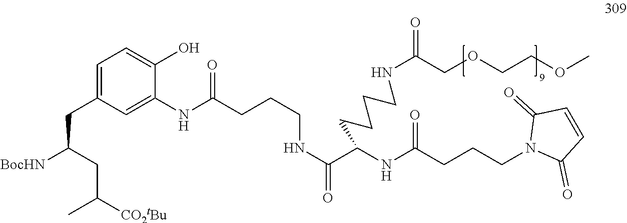

To a solution of compound 308 (105 g, 97.1 mmol, 1.0 eq.) in EtOH (5.3 L) was added N-succinimidyl 4-maleimido-butyrate (54.4 g, 194.2 mmol, 2.0 eq.) at r.t. Then 0.1M NaH₂PO₄ solution (1.1 L) was added, and the reaction mixture was stirred at r.t. overnight. EtOH was then evaporated under vacuum and the residue was poured onto water (3 L). The aqueous solution was extracted with ethyl acetate (4×500 mL), the organic layers were combined and washed with brine (2 L), dried over sodium sulfate, concentrated and the crude product was purified by silica gel column with a gradient of MeOH/DCM to give the title compound (100 g, 83% yield) as a yellow oil. ¹H NMR (400 MHz, CDCl₃) δ 9.43 (s, 1H), 7.35 (s, 1H), 7.23 (t, J=5.1 Hz, 1H), 7.01 (d, J=4.5 Hz, 2H), 6.89 (s, 2H), 6.70 (s, 2H), 4.56-4.45 (m, 1H), 4.30 (t, J=9.7 Hz, 1H), 3.97 (s, 2H), 3.86-3.74 (m, 1H), 3.66-3.63 (m, 36H), 3.58-3.52 (m, 5H), 3.38 (s, 3H), 3.33-3.19 (m, 3H), 2.47 (d, J=6.2 Hz, 4H), 2.23 (dd, J=11.6, 6.1 Hz, 2H), 1.91 (dtd, J=26.8, 13.6, 6.5 Hz, 7H), 1.71 (d, J=7.7 Hz, 2H), 1.56-1.49 (m, 2H), 1.42 (s, 9H), 1.39 (s, 9H), 1.10 (d, J=6.5 Hz, 3H). ESI m/z calcd. for C₆₀H₁₀₁N₆O₂₁ [M+H]⁺: 1241.69, found 1241.69.

### Example 350. Synthesis of (2S, 4R)-4-((tert-butoxycarbonyl)amino)-5-(4-hydroxy-3-nitrophenyl)-2-methylpentanoic acid (310)

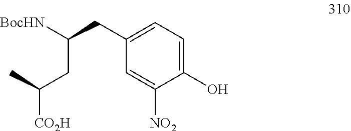 [SMILES: C[C@@H](C[C@H](Cc1ccc(O)c([N+](=O)[O-])c1)NC(=O)OC(C)(C)C)C(=O)O]

To a solution of (2S, 4R)-4-((tert-butoxycarbonyl)amino)-5-(4-hydroxyphenyl)-2-methyl-pentanoic acid (0.57 g, 1.76 mmol, 1.0 eq.) in THF (10 mL)was added t-BuONO (0.63 mL, 5.28 mmol, 3.0 eq.) at 0° C. The reaction was stirred at 0° C. for 1 hr then room temperature 1 h. After water (50 mL) was added, the reaction mixture was extracted with ethyl acetate (3×30 mL). The combined organic layers were washed with brine (100 mL), dried over anhydrous sodium sulfate, filtered, concentrated and purified by silica gel column chromatography (2:1 hexanes/ethyl acetate, containing 1% HOAc) to give the title compound as a yellow solid (0.50 g, 77% yield). ¹H NMR (400 MHz, DMSO) 67.92 (s, 1H), 7.47 (d, J=8.3 Hz, 1H), 7.05 (d, J=8.5 Hz, 1H), 3.73 (s, 1H), 2.78 (dd, J=13.6, 5.3 Hz, 1H), 2.69-2.47 (m, 2H), 1.87 (t, J=11.9 Hz, 1H), 1.47-1.37 (m, 1H), 1.32 (s, 9H), 1.17 (d, J=7.2 Hz, 3H). ESI MS m/z calcd. for C₁₇H₂₅N₂O₇ [M+H]⁺ 369.15, found 369.14.

### Example 351. Synthesis of (2S, 4R)-5-(3-amino-4-hydroxyphenyl)-4-((tert-butoxy-carbonyl)amino)-2-methylpentanoic acid (311)

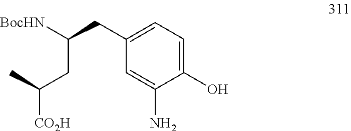 [SMILES: C[C@@H](C[C@H](Cc1ccc(O)c(N)c1)NC(=O)OC(C)(C)C)C(=O)O]

A mixture of compound 310 (0.50 g, 1.36 mmol, 1.0 eq.) and Pd/C (10 wt %, 0.02 g) in methanol (10 mL) was hydrogenated (1 atm H₂) at r.t. for 1 h, and then filtered through Celite (filter aid). The filtrate was concentrated to afford the title compound as a white foam (0.43 g, 93% yield). ESI MS m/z calcd. for C₁₇H₂₇N₂O₅[M+H]⁺ 339.18, found 339.17. ¹H NMR (400 MHz, MeOD) δ 6.60 (d, J=7.9 Hz, 2H), 6.44 (d, J=7.3 Hz, 1H), 3.71 (d, J=6.3 Hz, 1H), 2.62-2.37 (m, 3H), 1.83 (ddd, J=13.7, 9.9, 3.7 Hz, 1H), 1.39 (s, 9H), 1.13 (d, J=7.1 Hz, 3H).

### Example 352. Synthesis of(2S,4R)-4-((tert-butoxycarbonyl)amino)-5-(3-((S)-34-(4-(2,5-dioxo-2,5-dihydro-1H-pyrrol-1-yl)butanamido)-28,35,38-trioxo-2,5,8,11,14,17,20,23,26-nonaoxa-29,36,39-triazatritetracontan-43-amido)-4-hydroxyphenyl)-2-methylpentanoic acid (313)

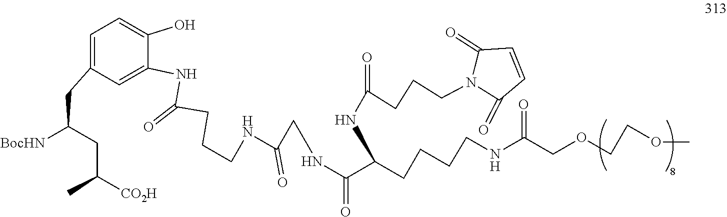

To a solution of compound 311 (78.0 g, 85.0 mmol, 1.0 eq.) and 2,5-dioxopyrrolidin-1-yl (S)-34-(4-(2,5-dioxo-2,5-dihydro-1H-pyrrol-1-yl)butanamido)-28,35,38-trioxo-2,5,8,11,14,17,20,23,26-nonaoxa-29,36,39-triazatritetracontan-43-oate (93.3 g, 95.8 mmol, 1.1 eq.) in 95% EtOH (3.3 L) at room temperature was added NaH₂PO₄ (0.1 M, 660 mL). The reaction was stirred at r.t. overnight then diluted with dichloromethane and washed with brine, dried over anhydrous sodium sulfate, filtered and concentrated. The residue was purified by column chromatography (3-10% MeOH/DCM) to give the title compound as a yellow oil (43 g, 37% yield).

### Example 353. Synthesis of(2S,4R)-4-amino-5-(3-((S)-34-(4-(2,5-dioxo-2,5-dihydro-1H-pyrrol-1-yl)butanamido)-28,35,38-trioxo-2,5,8,11,14,17,20,23,26-nonaoxa-29,36,39-triazatritetracontan-43-amido)-4-hydroxyphenyl)-2-methylpentanoic acid (314)

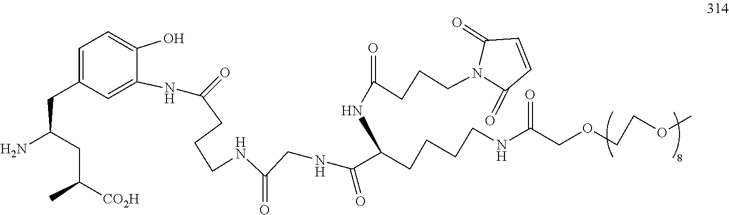

A solution of compound 313 (2.25 g, 1.78 mmol) in dioxane (10 mL) was treated with HCl (con. 3 mL) at r.t. for 1 h, then concentrated and co-evaporated with toluene/ethanol to give crude title product (1.97 g, 100% yield), which was used directly in the next step. ESI m/z calcd. for C₅₃H₈₈N₇O₂₀ [M+H]⁺: 1142.6085, found: 1142.6140.

### Example 354. Synthesis of (S, Z)-tert-butyl 4-((tert-butoxycarbonyl)amino)-2-methyl-5-phenylpent-2-enoate (315)

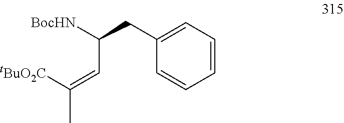 [SMILES: C/C(=C/[C@H](Cc1ccccc1)NC(=O)OC(C)(C)C)C(=O)OC(C)(C)C]

(S)-tert-butyl (1-oxo-3-phenylpropan-2-yl)carbamate (1.01 g, 4.0 mmol) was dissolved in dry dichloromethane (50 mL), to which tert-butyl 2-(triphenyl-phosphoranylidene)-propanoate (3.20 g, 8 mmol) was added and the solution was stirred at r.t. for 1.5 h as determined complete by TLC. Purification by column chromatography (10-50% EtOAc/hexanes) afforded the title compound (1.38 g, 96% yield). ESI m/z calcd. for C₂₁H₃₁NO₄ [M+H]⁺: 362.2332, found: 362.2350.

### Example 355. Synthesis of (S, E)-tert-butyl 4-((tert-butoxycarbonyl)amino)-2-methyl-5-(4-nitrophenyl)pent-2-enoate (316) and (S, E)-tert-butyl 4-((tert-butoxycarbonyl)amino)-2-methyl-5-(2-nitrophenyl)pent-2-enoate (317)

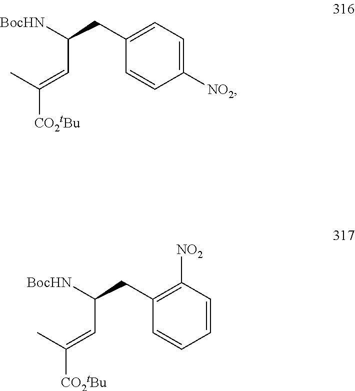 [SMILES: C/C(=C\[C@H](Cc1ccc([N+](=O)[O-])cc1)NC(=O)OC(C)(C)C)C(=O)OC(C)(C)C ; C/C(=C\[C@H](Cc1ccccc1[N+](=O)[O-])NC(=O)OC(C)(C)C)C(=O)OC(C)(C)C]

(S, Z)-tert-butyl 4-((tert-butoxycarbonyl)amino)-2-methyl-5-phenylpent-2-enoate (1.320 g, 3.65 mmol.) was dissolved in THF (45 mL), to which a solution of tert-butyl nitrite (1.151 g, 10.95 mmol) in THF (6 mL) was added. The reaction was stirred at r.t. for 3 h and then poured onto water, extracted with ethyl acetate (3×50 mL) and the combined organic phases were washed with brine (50 mL), dried over anhydrous sodium sulfate, filtered and concentrated. Purification by column chromatography (10-50% EtOAc/hexanes) afforded 316 (907 mg, 61% yield), ESI m/z calcd. for C₂₁H₃₁N₂O₆ [M+H]⁺: 407.2183, found: 407.2230, and 317 (133 mg, 9.0% yield), ESI m/z calcd. for C₂₁H₃₁N₂O₆ [M+H]⁺: 407.2183, found: 407.2245.

### Example 356. Synthesis of (2S, 4R)-tert-butyl 5-(4-aminophenyl)-4-((tert-butoxy-carbonyl)amino)-2-methylpentanoate (318)

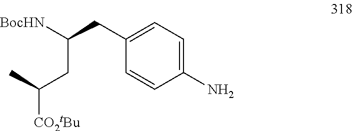 [SMILES: C[C@@H](C[C@H](Cc1ccc(N)cc1)NC(=O)OC(C)(C)C)C(=O)OC(C)(C)C]

A stirred mixture of (S, E)-tert-butyl 4-((tert-butoxycarbonyl)amino)-2-methyl-5-(4-nitrophenyl)pent-2-enoate (15.0 g, 36.9 mmol, 1.0 eq.), chiral spiro iridium catalyst (1.50 g, 0.78 mmol) (Zhu, S.-F.; et al, J. Am. Chem. Soc. 2006, 128, 12886; Song, S., et al, Org. Lett., 2013, 15, 3722) and Et₃N (4.26 g, 41.0 mmol) in 150 mL of methanol in a hydrogenation vessel was charged 6 atm of H₂ at 60° C. for 20 h. After releasing hydrogen, the mixture was concentrated and purified with C-8 column eluted with water/methanol (5% methanol to 50% methanol) to give 12.8 g (yield 92%) of the title compound which was used directly in the next step. ESI m/z calcd. for C₂₁H₃₅N₂O₄ [M+H]⁺: 379.2578, found: 379.2610.

### Example 357. Synthesis of (2S, 4R)-tert-butyl 4-((tert-butoxycarbonyl)amino)-5-(4-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)phenyl)-2-methylpentanoate (319)

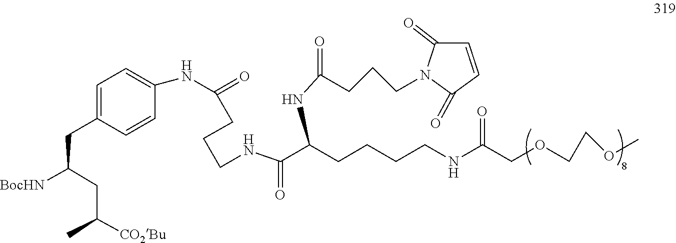

(2S, 4R)-tert-butyl 5-(4-aminophenyl)-4-((tert-butoxy-carbonyl)amino)-2-methylpentanoate (3.511 g, 9.28 mmol) and (S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontan-40-oic acid (7.613 g, 9.28 mmol) in DCM (75 mL) was added EDC (3.50 g, 18.23 mmol). The mixture was stirred for 12 hours, concentrated, purified on silica gel column eluted with 30% EtOAc/DCM, pooled the fractions, evaporated with oil pump to afford the title compound (8.98 g, 82% yield). ESI m/z calcd. for C₅₈H₉₇N₆O₂₉ [M+H]⁺: 1181.6809, found: 1181.6880.

### Example 358. Synthesis of (2S, 4R)-4-amino-5-(4-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontanamido)phenyl)-2-methylpentanoic acid (320)

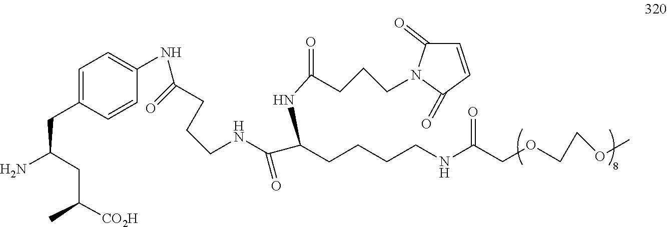

A solution of compound 319 (2.01 g, 1.70 mmol) in dioxane (10 mL) was treated with con. HCl (3 mL) at r.t. for 1 h, then concentrated and co-evaporated with toluene/ethanol to give crude title product (1.82 g, 100% yield), which was used directly in the next step. ESI m/z calcd. for C₅₁H₈₅N₆O₁₈ [M+H]⁺: 1069.5921, found: 1069.5990.

### Example 359. Synthesis of (2S, 4R)-tert-butyl 4-((tert-butoxycarbonyl)amino)-5-(4-((S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29-decaoxa-32, 39, 42-triazahexatetracontanamido)phenyl)-2-methylpentanoate (321)

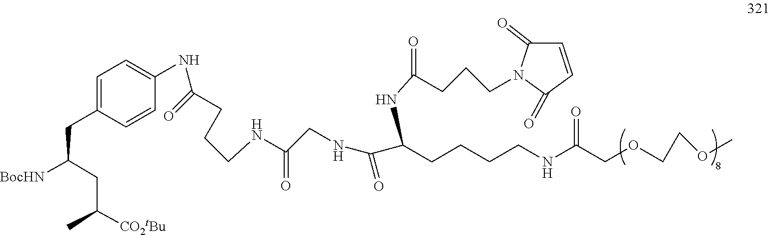

(2S, 4R)-tert-butyl 5-(4-aminophenyl)-4-((tert-butoxy-carbonyl)amino)-2-methyl pentanoate (3.012 g, 7.96 mmol) and (S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29-decaoxa-32, 39, 42-triazahexatetracontan-46-oic acid (7.346 g, 9.97 mmol) in DCM (75 mL) was added EDC (3.50 g, 18.23 mmol). The mixture was stirred for 12 hours, concentrated, purified on silica gel column eluted with 30% EtOAc/DCM, pooled the fractions, evaporated with oil pump to afford the title compound (8.672 g, 85% yield). ESI m/z calcd. for C₆₂H₁₀₄N₇O₂₁ [M+H]⁺: 1282.7286, found: 1282.7365.

### Example 360. Synthesis of (2S, 4R)-4-amino-5-(4-((S)-37-(4-(2, 5-dioxo-2, 5-dihydro-1H-pyrrol-1-yl)butanamido)-31, 38, 41-trioxo-2, 5, 8, 11, 14, 17, 20, 23, 26, 29-decaoxa-32, 39, 42-triazahexatetracontanamido)phenyl)-2-methylpentanoic acid (322)

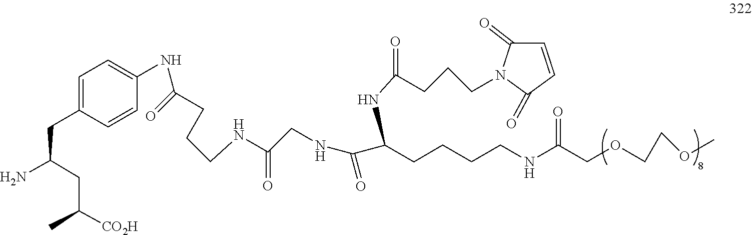

A solution of compound 321 (1.951 g, 1.52 mmol) in dioxane (10 mL) was treated with con. HCl (3 mL) at r.t. for 1 h, then concentrated and co-evaporated with toluene/ethanol to give crude title product (1.71 g, 100% yield), which was used directly in the next step. ESI m/z calcd. for C₅₃H₈₈N₇O₁₉ [M+H]⁺: 1126.6136, found: 1126.6265.

### Example 361. Synthesis of perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (323)

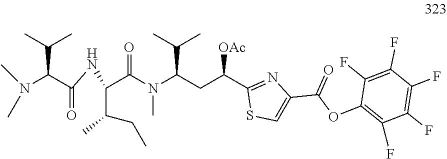 [SMILES: CC[C@H](C)[C@H](NC(=O)[C@H](C(C)C)N(C)C)C(=O)N(C)[C@H](C[C@@H](OC(C)=O)c1nc(C(=O)Oc2c(F)c(F)c(F)c(F)c2F)cs1)C(C)C]

To a solution of 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-1I1-yl)thiazole-4-carboxylic acid (2.210 g, 4.090 mmol) and pentafluorophenol (1.00 g, 5.430 mmol) in dichloromethane (60 mL) were added EDC (1.580 g, 8.22 mmol). The reaction mixture was stirred overnight. After the solvent was removed under reduced pressure, the reaction mixture was concentrated and purified on silica gel column chromatography (1:15 to 1:4 EtOAc/DCM) to afford the title compound (2.455 g, 85% yield), which was used directly for the next step. ESI MS m/z calcd. for C₃₂H₄₄F₅N₄06S [M+H]⁺ 707.2902, found 707.2970.

### Example 362. Synthesis of perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (324)

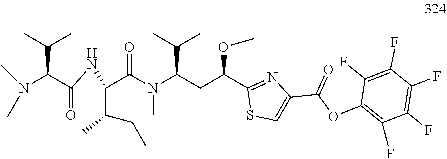 [SMILES: CC[C@H](C)[C@H](NC(=O)[C@H](C(C)C)N(C)C)C(=O)N(C)[C@H](C[C@@H](OC)c1nc(C(=O)Oc2c(F)c(F)c(F)c(F)c2F)cs1)C(C)C]

To a solution of 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylic acid (2.285 g, 4.460 mmol) and pentafluorophenol (1.00 g, 5.430 mmol) in dichloromethane (60 mL) were added EDC (1.580 g, 8.22 mmol). The reaction mixture was stirred overnight. After the solvent was removed under reduced pressure, the reaction mixture was concentrated and purified on silica gel column chromatography (1:15 to 1:4 EtOAc/DCM) to afford the title compound (2.510 g, 83% yield), which was used directly for the next step. ESI MS m/z calcd. for C₃₁H₄₄F₅N₄05S [M+H]⁺ 679.2953, found 679.2995.

### Example 363. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(4-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)phenyl)-2-methylpentanoic acid (325)

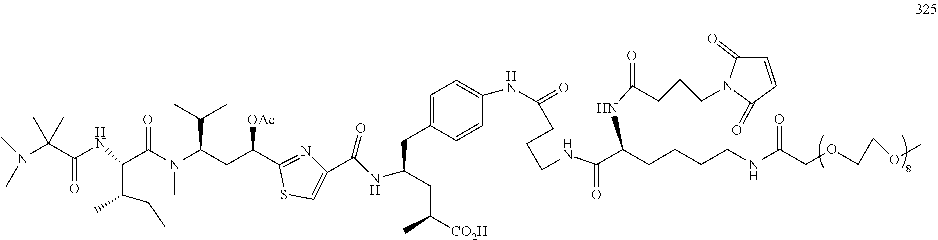

A mixture of perfluorophenyl 2-((6S, 9R, TTR)-6-((S)-sec-butyl)-9-isopropyl-2,3,3,8-tetramethy l-4,7,13-trioxo-12-oxa-2,5,8-triazatetradecan-11-yl)thiazole-4-carboxylate (1.001 g, 1.444 mmol) and compound 320 (1.543 g, 1.442 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.437 g, 65% yield). ESI MS m/z calcd. for C₇₄H₁₂1N₁₀O₂₂S [M+H]⁺1533.8378, found 1533.8470.

Example 364. Synthesis of (2S, 4R)-4-(2-((6S, 9R, 11R)-6-((S)-sec-butyl)-9-isopropyl- 2,3,3,8- tetramethyl-4,7-dioxo-12-oxa-2,5,8-triazatridecan-11-yl)thiazole-4-carboxamido)-5-(4-((S)-34-(4-(2,5-dioxo-2, 5-dihydro-1H-pyrrol-1-yl)butanamido)-28, 35-dioxo-2,5,8,11,14,17,20,23,26- nonaoxa-29, 36-diazatetracontanamido)phenyl)-2-methylpentanoic acid (326)

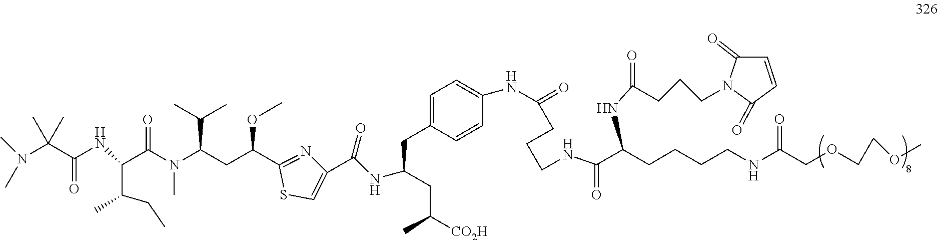

A mixture of perfluorophenyl 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylate (1.011 g, 1.521 mmol) and compound 320 (1.550 g, 1.444 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.401 g, 63% yield). ESI MS m/z calcd. for C₇₃H₁₂₁N₁₀O₂₁ S [M+H]⁺ 1505.8429, found 1505.8510.

### Example 365. Synthesis of (2S, 4R)-4-(2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxamido)-5-(4-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)phenyl)-2-methylpentanoic acid (327)

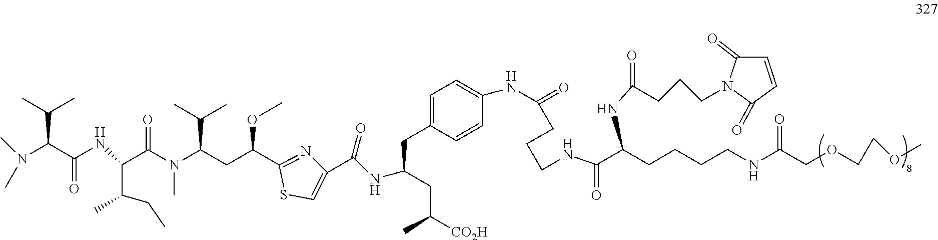

A mixture of perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (1.015 g, 1.496 mmol) and compound 320 (1.545 g, 1.443 mmol), and DIPEA (0.6 mL) in DMF (30 mL) was stirred at room temperature overnight. The reaction was concentrated under high vacuum, dissolved in small amount of water, then purified by prep-HPLC (C18 column, 10-90% MeCN/H₂O), pooled the fractions containing the product, concentrated and lyophilized to give the title compound (1.357 g, 61% yield). ESI MS m/z calcd. for C₇₄H₁₂₃N₁₀O₂₁S [M+H]⁺ 1519.8586, found 1519.8650.

### Example 366. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(4-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)phenyl)-2-methylpentanoic acid (328)

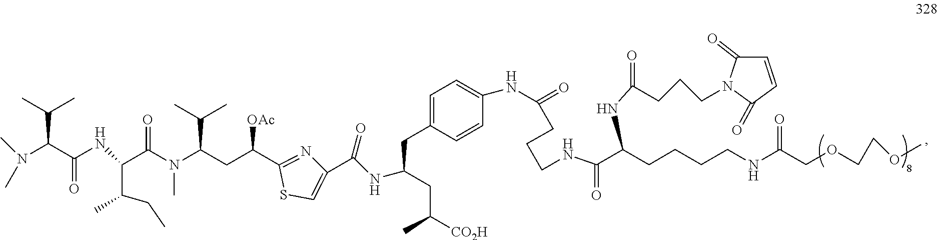

A mixture of perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (1.020 g, 1.444 mmol) and compound 320 (1.540 g, 1.442 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.338 g, 60% yield). ESI MS m/z calcd. for C₇₅H₁₂₃N₁₀O₂₂S [M+H]⁺ 1547.8535, found 1547.8595.

### Example 367. Synthesis of (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-5-(4-((S)-34-(4-(2, 5-dioxo-2, 5-dihydro-1H-pyrrol-1-yl)butanamido)-28, 35, 38-trioxo-2, 5, 8, 11, 14, 17, 20, 23, 26-nonaoxa-29, 36, 39-triazatritetracontanamido)phenyl)-2-methylpentanoic acid (329)

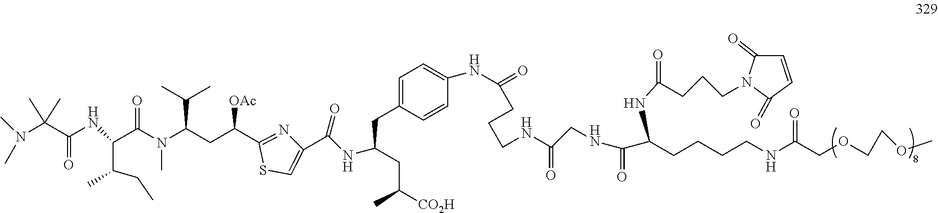

A mixture of perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (1.003 g, 1.444 mmol) and compound 322 (1.535 g, 1.363 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.365 g, 65% yield). ESI MS m/z calcd. for C₇₆H₁₂₄N₁₁O₂₃S [M+H]⁺ 1590.8593, found 1590.8670.

### Example 368. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(4-((S)-34-(4-(2, 5-dioxo-2, 5-dihydro-1H-pyrrol-1-yl)butanamido)-28, 35, 38-trioxo-2, 5, 8, 11, 14, 17, 20, 23, 26-nonaoxa-29, 36, 39-triazatritetracontanamido)phenyl)-2-methylpentanoic acid (330)

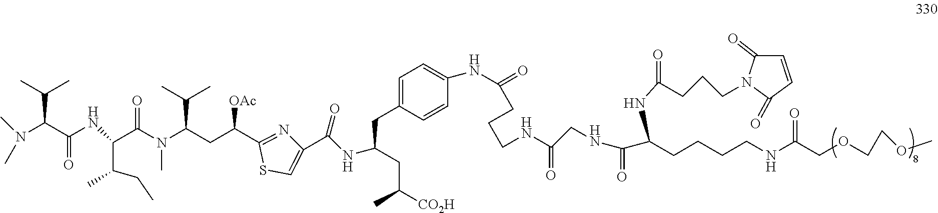

A mixture of perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (1.020 g, 1.444 mmol) and compound 322 (1.540 g, 1.367 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.315 g, 60% yield). ESI MS m/z calcd. for C₇₇H₁₂₆N₁₁O₂₃S [M+H]⁺ 1604.8750, found 1604.8835.

### Example 369. Synthesis of (2S, 4R)-4-(2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxamido)-5-(3-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)-4-hydroxyphenyl)-2-methylpentanoic acid (331)

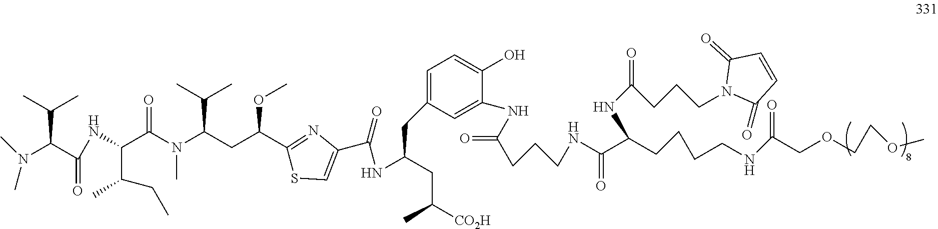

A mixture of perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (1.015 g, 1.496 mmol) and compound 314 (1.458 g, 1.401 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.338 g, 62% yield). ESI MS m/z calcd. for C₇₄H₁₂₃N₁₀O₂₂S [M+H]⁺ 1535.8535, found 1535.8655.

### Example 370. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)-4-hydroxyphenyl)-2-methylpentanoic acid (332)

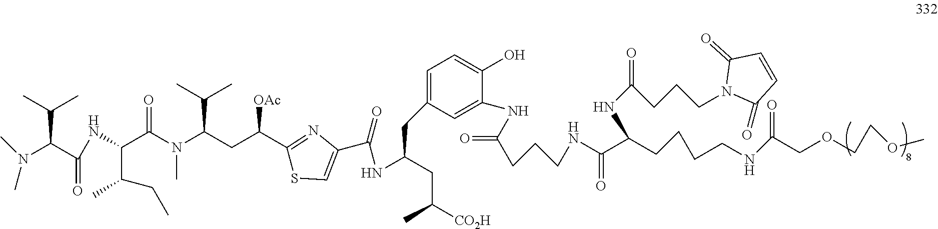

A mixture of perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (1.020 g, 1.444 mmol) and (2S, 4R)-4-amino-5-(3-((S)-34-(4-(2, 5-dioxo-2, 5-dihydro-1H-pyrrol-1-yl)butanamido)-28, 35-dioxo-2, 5, 8, 11, 14, 17, 20, 23, 26-nonaoxa-29, 36-diazatetracontanamido)-4-hydroxyphenyl)-2-methylpentanoic acid (1.455 g, 1.395 mmol), and DIPEA (0.5 mL) in DMF (35 mL) was stirred at room temperature overnight. The reaction was concentrated under high vacuum, dissolved in small amount of water and then purified by prep-HPLC (C₁₈ column, 10-90% MeCN/H₂O), pooled the fractions containing the product, concentrated and lyophilized to give the title compound (1.333 g, 61% yield). ESI MS m/z calcd. for C₇₅H₁₂₃N₁₀O₂₃S [M+H]⁺ 1563.8484, found 1563.8550.

### Example 371. Synthesis of (2S, 4R)-4-(2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxamido)-5-(3-((S)-34-(4-(2, 5-dioxo-2, 5-dihydro-1H-pyrrol-1-yl)butanamido)-28, 35, 38-trioxo-2, 5, 8, 11, 14, 17, 20, 23, 26-nonaoxa-29, 36, 39-triazatritetracontanamido)-4-hydroxyphenyl)-2-methylpentanoic acid (333)

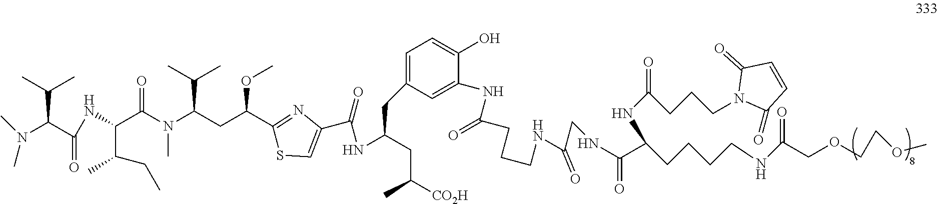

A mixture of perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (1.015 g, 1.496 mmol) and compound 314 (1.523 g, 1.387 mmol), and DIPEA (0.6 mL) in DMF (30 mL) was stirred at room temperature overnight. The reaction was concentrated under high vacuum, dissolved in small amount of water, then purified by prep-HPLC (C₁₈ column, 10-90% MeCN/H₂O), pooled the fractions containing the product, concentrated and lyophilized to give the title compound (1.326 g, 60% yield). ESI MS m/z calcd. for C₇₆H₁₂₆N₁₁O₂₃S [M+H]⁺ 1592.8750, found 1592.8845.

### Example 372. Synthesis of (2S, 4R)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-5-(3-((S)-34-(4-(2, 5-dioxo-2, 5-dihydro-1H-pyrrol-1-yl)butanamido)-28, 35, 38-trioxo-2, 5, 8, 11, 14, 17, 20, 23, 26-nonaoxa-29, 36, 39-triazatritetracontanamido)-4-hydroxyphenyl)-2-methylpentanoic acid (334)

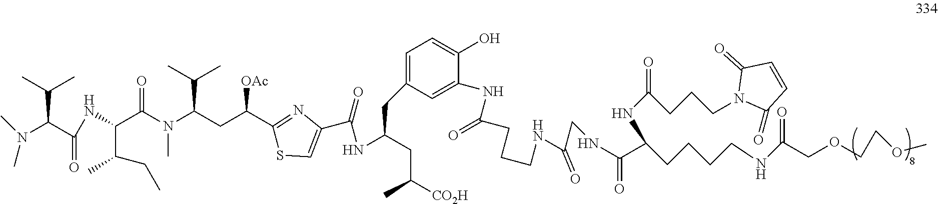

A mixture of perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (1.020 g, 1.444 mmol) and compound 314 (1.520 g, 1.384 mmol), and DIPEA (0.5 mL) in DMF (35 mL) was stirred at room temperature overnight. The reaction was concentrated under high vacuum, dissolved in small amount of water and then purified by prep-HPLC (C₁₈ column, 10-90% MeCN/H₂O), pooled the fractions containing the product, concentrated and lyophilized to give the title compound (1.391 g, 62% yield). ESI MS m/z calcd. for C₇₇H₁₂₆N₁₁O₂₄S [M+H]⁺ 1620.8699, found 1620.8790.

### Example 373. Synthesis of (2S, 4R)-5-(3-(13-(2′, 5′, 8′, 11′, 14′, 17′, 20′, 23′-octaoxapentacosane-25′-sulfonyl)-15-(2, 5-dioxo-2, 5-dihydro-1H-pyrrol-1-yl)-4, 7, 10-trioxo-3, 6, 9, 13-tetraazapentadecanamido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoic acid (335)

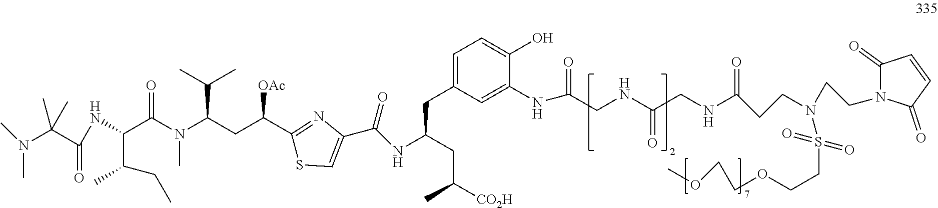

A solution of (2S, 4R)-5-(3-amino-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-2-methylpentanoic acid, HCl salt (Tub-039, R. Zhao, et al, PCT/CN2017/120454; R. Zhao, et al, 14th PEGS Boston, Boston, MA, USA, 3 May 2018) (83 mg, 0.106 mmol) and compound 110 (122 mg, 0.134 mmol) in DMF (8 mL) at 0° C., DIPEA (2 mL) was added. The reaction mixture was stirred at 0° C. for 0.5 h, followed by at room temperature for 4 h. Then the reaction mixture was concentrated, and purified by prep-HPLC (mobile phase: acetonitrile/water=10% to 80% with 0.1% formic acid) to afford the title product (95.5 mg, 58% yield). MS-ESI m/z: [M+H]⁺ calcd. for C₆₉H₁₁₂N₁₁O₂₄S, 1542.72; found, 1542.73.

### Example 374. Synthesis of 3, 3′-((2-(2-(bis(2-(2, 5-dioxo-2, 5-dihydro-1H-pyrrol-1-yl)ethyl)amino)-2-oxoethoxy)acetyl)azanediyl)dipropanoic acid (336)

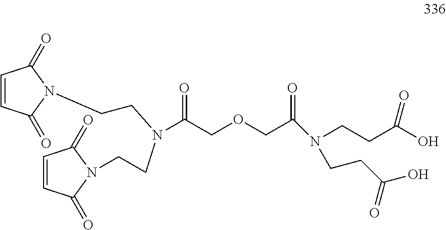 [SMILES: O=C(O)CCN(CCC(=O)O)C(=O)COCC(=O)N(CCN1C(=O)C=CC1=O)CCN1C(=O)C=CC1=O]

To a solution of 3, 3′-azanediyldipropanoic acid (1.00 g, 6.20 mmol) in DMA (25 mL) and NaH₂PO₄ buffer (30 mL, 100 mM, pH 7.0) was added 2, 5-dioxopyrrolidin-1-yl 2-(2-(bis(2-(2, 5-dioxo-2, 5-dihydro-1H-pyrrol-1-yl)ethyl)amino)-2-oxoethoxy)acetate (3.00 g, 6.30 mmol). The mixture then was stirred at r.t. for 4 h, concentrated, purified on silica gel column eluted with H₂O/CH₃CN (3% H₂O in CH₃CN to 5% H₂O in CH₃CN) to afford the title compound (2.52 g, 78% yield). ESI MS m/z C₂₂H₂₇N₄O₁₁ [M+H]⁺, calcd. 523.17, found 523.20.

### Example 375. Synthesis of bis(2, 5-dioxopyrrolidin-1-yl) 3, 3′-((2-(2-(bis(2-(2, 5-dioxo-2, 5-dihydro-1H-pyrrol-1-yl)ethyl)amino)-2-oxoethoxy)acetyl)azanediyl) dipropanoate (337)

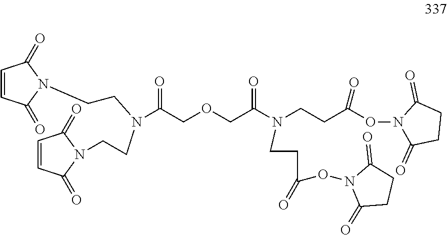 [SMILES: O=C(CCN(CCC(=O)ON1C(=O)CCC1=O)C(=O)COCC(=O)N(CCN1C(=O)C=CC1=O)CCN1C(=O)C=CC1=O)ON1C(=O)CCC1=O]

In the mixture solution of compound 336 (1.20 g, 2.30 mmol) and N-hydroxysuccinimide (0.34 g, 2.95 mmol) in dry DMA (30 mL) was added EDC (1.00 g, 5.23 mmol). The reaction mixture was stirred for 4 h, then concentrated and purified by silica gel column chromatography (4:1 to 5:3 DCM/EtOAc) to give the title compound (1.26 g, 77% yield). ESI MS m/z: calcd. for C₃₀H₃₃N₆O₁₅ [M+H]⁺ 717.20, found 717.30.

### Example 376. Synthesis of 3, 3′-((4-(bis(2-(2, 5-dioxo-2, 5-dihydro-1H-pyrrol-1-yl)ethyl)amino)-4-oxobutanoyl)azanediyl)dipropanoic acid (338)

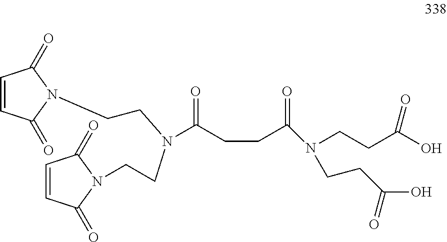 [SMILES: O=C(O)CCN(CCC(=O)O)C(=O)CCC(=O)N(CCN1C(=O)C=CC1=O)CCN1C(=O)C=CC1=O]

To a solution of 3, 3′-azanediyldipropanoic acid (1.00 g, 6.20 mmol) in DMA (25 mL) and NaH₂PO₄ buffer (30 mL, 100 mM, pH 7.0) was added 2, 5-dioxopyrrolidin-1-yl 4-(bis(2-(2, 5-dioxo-2, 5-dihydro-1H-pyrrol-1-yl)ethyl)amino)-4-oxobutanoate (2.90 g, 6.30 mmol). The mixture then was stirred at r.t. for 4 h, concentrated, purified on silica gel column eluted with H₂O/CH₃CN (3% H₂O in CH₃CN to 5% H₂O in CH₃CN) to afford the title compound (2.51 g, 80% yield). ESI MS m/z C₂₂H₂₇N₄O₁₀ [M+H]⁺, calcd. 507.17, found 507.20.

### Example 377. Synthesis of bis(2, 5-dioxopyrrolidin-1-yl) 3, 3′-((4-(bis(2-(2, 5-dioxo-2, 5-dihydro-1H-pyrrol-1-yl)ethyl)amino)-4-oxobutanoyl)azanediyl)dipropanoate (339)

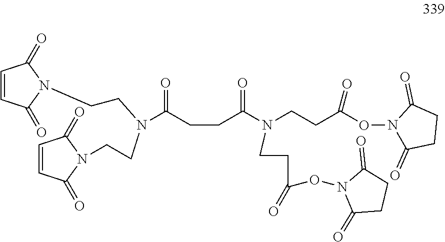 [SMILES: O=C(CCN(CCC(=O)ON1C(=O)CCC1=O)C(=O)CCC(=O)N(CCN1C(=O)C=CC1=O)CCN1C(=O)C=CC1=O)ON1C(=O)CCC1=O]

To a mixture solution of compound 338 (1.15 g, 2.27 mmol) and N-hydroxy succinimide (0.34 g, 2.95 mmol) in dry DMA (30 mL) was added EDC (1.00 g, 5.23 mmol). The reaction mixture was stirred for 4 h, then concentrated and purified by silica gel column chromatography (4:1 to 5:3 DCM/EtOAc) to give the title compound (1.27 g, 80% yield). ESI MS m/z: calcd. for C₃₀H₃₃N₆O₁₄ [M+H]⁺ 701.20, found 701.30.

### Example 378. Synthesis of (2R, 3R)-2, 3-bis(((benzyloxy)carbonyl)amino)-4-((4-(tert-butoxy)-4-oxobutyl)amino)-4-oxobutanoic acid (340)

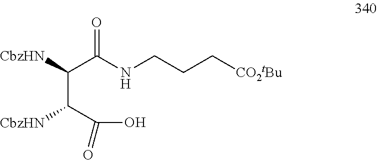 [SMILES: CC(C)(C)OC(=O)CCCNC(=O)[C@H](NC(=O)OCc1ccccc1)[C@@H](NC(=O)OCc1ccccc1)C(=O)O]

To a mixture of dibenzyl ((3R, 4S)-2, 5-dioxotetrahydrofuran-3, 4-diyl)-dicarbamate (4.25 g, 10.68 mmol, 1.0 eq.) and DMAP (13 mg, 0.11 mmol, 0.01 eq.) in 20 mL of dry DCM, a solution of t-butyl aminobutyrate (1.78 g, 11.21 mmol, 1.05 eq.) in 10 mL of anhydrous DCM was added. After the addition was completed, the starting material was completely dissolved and the reaction was allowed to stir at r.t. overnight. The crude product was loaded on a silica gel column and eluted with 3-5% MeOH/DCM. Fractions were combined and evaporated, the residue was triturated with PE/DCM (1:1) to afford 3.3 g of a white solid (yield 55.9%). ESI m/z calcd. for C₂₈H₃₆N₃O₉ [M+H]⁺: 558.2, found: 558.2.

### Example 379. Synthesis of 2, 2-dimethyl-4-oxo-3, 8, 11, 15, 18-pentaoxa-5-azahenicosan-21-oic acid (341)

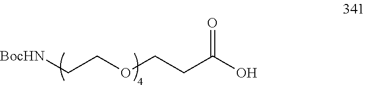

In a 500 mL flask, H₂N-PEG₄-CH₂CH₂CO₂H (3.0 g, 11.3 mmol, 1.0 eq.) and K₂CO₃ (4.7 g, 33.93 mmol, 3.0 eq.) were dissolved in 50 mL of water, and cooled over an ice water bath. Boc₂O (3.2 g, 14.7 mmol, 1.3) in 50 mL of THF was added dropwise. The reaction was allowed to warm to r.t. and stirred overnight. The reaction mixture was adjusted to pH 4-5 with 1N KHSO₄ and extracted with DCM (200 mL×1, 100 mL×3), washed with water (500 mL×1), and brine (500 mL×1), dried over anhydrous sodium sulfate, and concentrated. The residue was dissolved in a small amount of DCM and then loaded on a silica gel column, eluted with 2-4% MeOH/DCM, and the fractions were combined and concentrated to give 3.8 g of colorless oil (yield 93%). ESI m/z calcd. for C₁₆H₃₂NO₈ [M+H]⁺: 366.2, found: 366.2.

### Example 380. Synthesis of benzyl 2, 2-dimethyl-4-oxo-3, 8, 11, 15, 18-pentaoxa-5-azahenicosan-21-oate (342)

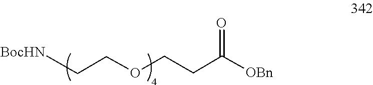

In a 50 mL single-necked flask, BocHN-PEG₄-CH₂CH₂CO₂H (0.81 g, 2.22 mmol, 1.0 eq.), K₂CO₃ (0.92 g, 6.66 mmol, 3.0 eq.) and NaI (0.033 g, 0.222 mmol, 0.1 eq.) were mixed in 10 mL of DMF, cooled over an ice water bath, and BnBr (0.57 g, 3.33 mmol, 1.5 eq.) was added dropwise, and the mixture was warmed to r.t. and stirred overnight. The reaction mixture was diluted with 100 mL of water, extracted with DCM (100 mL×2), washed with water (200 mL×1), and brine (200 mL×1), dry over anhydrous sodium sulfate, and concentrated. The residue was dissolved in a small amount of DCM, loaded on silica gel column, eluted with is 70-90% EA/PE to give 0.69 g of colorless oil (69% yield). ESI m/z calcd. for C₂₃H₃₈NO₈ [M+H]⁺: 446.3, found: 446.3.

### Example 381. Synthesis of benzyl 1-amino-3, 6, 10, 13-tetraoxahexadecan-16-oate (343)

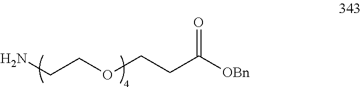

A solution of BocHN-PEG₄-CH₂CH₂CO₂Bn (0.69 g, 1.5 mmol, 1.0 eq.) in 6 mL of DCM and 3 mL of TFA was stirred at r.t. for 30 min. The solvents were removed and the residue was co-evaporated with DCM for three times, placed on high vacuum pump. The crude product was used directly in the next reaction. ESI m/z calcd. for C₁₈H₃₀NO₆ [M+H]⁺: 356.2, found: 356.2.

### Example 382. Synthesis of 2, 5-dioxopyrrolidin-1-yl 1-amino-3, 6, 10, 13-tetraoxahexadecan-16-oate (344)

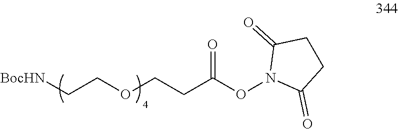

To a solution of BocHN-PEG₄-CH₂CH₂CO₂H (3.8 g, 10.4 mmol, 1.0 eq.) in 50 mL of dry DCM, NHS (1.4 g, 12.5 mmol, 1.2 eq.) and EDC (10.0 g, 52.0 mmol, 5.0 eq.) were added. The reaction was stirred at r.t. overnight and then washed with water (50 mL×2), brine (100 mL×1), dried over anhydrous sodium sulfate, and concentrated. The crude product was used directly in the next step. ESI m/z calcd. for C₂₀H₃₅N₂O₁₀ [M+H]⁺: 463.2, found:463.2.

### Example 383. Synthesis of 2, 2-dimethyl-4, 20-dioxo-3, 8, 11, 14, 17, 24, 27, 30, 33-nonaoxa-5, 21-diazahexatriacontan-36-oic acid (345)

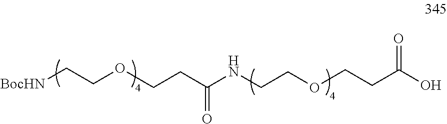

In a 300 mL flask, H₂N-PEG₄-CH₂CH₂CO₂H (2.8 g, 10.4 mmol, 1.0 eq.) and K₂CO₃ (4.3 g, 31.2 mmol, 3.0 eq.) were dissolved in 40 mL of water, cooled over an ice water bath, and the above crude NHS ester solution (3.8 g, 10.4 mmol, 1.0 eq.) in 40 mL of THF was added dropwise, and the mixture was warmed to r.t. and stirred overnight. The reaction mixture was adjusted to pH 4-5 using 1N KHSO₄, extracted with DCM (150 mL×1, 100 mL×2), washed with water (200 mL×1), and brine (200 mL×1), dried over anhydrous sodium sulfate, and concentrated. The residue was dissolved in small amount of DCM, and the loaded on a silica gel column, eluted with 4-6% MeOH/DCM to give a colorless oil (5.18 g, 81% yield). ESI m/z calcd. for C₂₇H₅₃N₂O₁₃ [M+H]⁺: 613.3, found: 613.3.

### Example 384. Synthesis of benzyl 2, 2-dimethyl-4, 20, 36-trioxo-3, 8, 11, 14, 17, 24, 27, 30, 33, 40, 43, 46, 49-tridecaoxa-5, 21, 37-triazadopentacontan-52-oate (346)

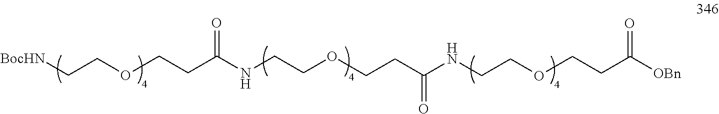

To a solution of H₂N-PEG₄-CH₂CH₂CO₂Bn (crude product from the previous step) dissolved in 3 mL of DMF, cooled over ice/water bath, DIPEA (0.78 g, 6.0 mmol, 4.0 eq.) was added dropwise, and followed by addition of a solution of 2, 2-dimethyl-4, 20-dioxo-3, 8, 11, 14, 17, 24, 27, 30, 33-nonaoxa-5, 21-diazahexatriacontan-36-oic acid (0.93 g, 1.5 mmol, 1.0 eq.) in 7 mL of DMF and HATU (1.72 g, 4.5 mmol, 3.0 eq.). The reaction was stirred over the ice bath for 2 hours, and diluted with 100 mL of water, extracted with DCM (100 mL×3), washed with 1N KHSO₄ (200 mL×1), saturated sodium bicarbonate (200 mL×1), and brine (200 mL×1), dried over anhydrous sodium sulfate, and concentrated. The residue was dissolved in a small amount of DCM, loaded on a silica gel column, and eluted 0-5% MeOH/DCM. Fractions were combined and concentrated to give 1.0 g of light yellow oil (71% yield). ESI m/z calcd. for C₄₅H₈₀N₃O₁₈ [M+H]⁺: 950.5, found: 950.5.

### Example 385. Synthesis of (50R, 51R)-1-benzyl 57-tert-butyl 50, 51-bis(((benzyloxy) carbonyl)amino)-17, 33, 49, 52-tetraoxo-4, 7, 10, 13, 20, 23, 26, 29, 36, 39, 42, 45-dodecaoxa-16, 32, 48, 53-tetraazaheptapentacontane-1, 57-dioate (347)

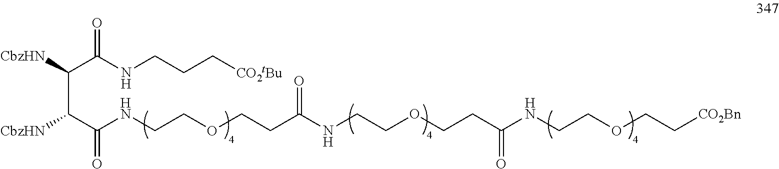

A solution of benzyl 2, 2-dimethyl-4, 20, 36-trioxo-3, 8, 11, 14, 17, 24, 27, 30, 33, 40, 43, 46, 49-tridecaoxa-5, 21, 37-triazadopentacontan-52-oate (1.0 g, 1.03 mmol, 1.0 eq.) in 6 mL of DCM, and 3 mL of TFA was stirred at r.t for 1 h. The solvents were removed and the residue was co-evaporated with DCM for three times, placed on high vacuum pump.

The crude product was re-dissolved in 10 mL of DMF, cooled over ice water bath. To which DIPEA (0.53 g, 4.12 mmol, 4.0 eq.), compound 340 (0.56 g, 1.03 mmol, 1.0 eq.) and HATU (1.17 g, 3.09 mmol, 3.0 eq.) were added in sequence. After stirring over the ice bath for 1 hour, 100 mL of water was added, and a solid precipitated out. The solid was collected by filtration and washed with water, dissolved in DCM, dried over anhydrous sodium sulfate, filtered and concentrated. The residue was dissolved in a small amount of DCM, loaded on a silica gel column, and eluted 0-10% MeOH/DCM. Fractions were combined and concentrated to give 0.93 g of light yellow foam (yield 65%). ESI m/z calcd. for C₆₈H₁₀₇N₈O₂₆ [M+H]: 1451.7, found: 1451.7.

### Example 386. Synthesis of (52R, 53R)-52, 53-bis(((benzyloxy)carbonyl)amino)-3, 19, 35, 51, 54-pentaoxo-1-phenyl-2, 6, 9, 12, 15, 22, 25, 28, 31, 38, 41, 44, 47-tridecaoxa-18, 34, 50, 55-tetraazanonapentacontan-59-oic acid (348)

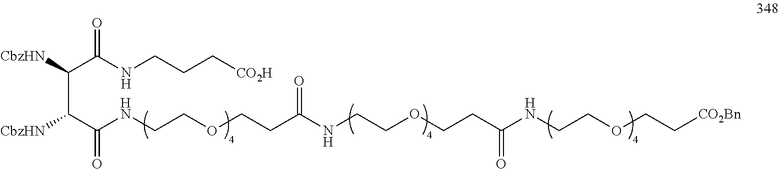

A solution of (50R, 51R)-1-benzyl 57-tert-butyl 50, 51-bis(((benzyloxy)carbonyl) amino)-17, 33, 49, 52-tetraoxo-4, 7, 10, 13, 20, 23, 26, 29, 36, 39, 42, 45-dodecaoxa-16, 32, 48, 53-tetraazaheptapentacontane-1, 57-dioate (0.93 g, 0.67 mmol, 1.0 eq.) in 6 mL of DCM, and 3 mL of TFA was stirred at r.t. for 1 h (the completion of the reaction was monitored by LC-MS). The solvents were removed and the residue was co-evaporated with DCM for three times, placed on high vacuum pump. The crude product was dissolved in a small amount of DCM and loaded on a silica gel column, and then eluted with 15-20% MeOH/DCM. Fractions were combined and concentrated to give 0.53 g of white foam (yield 60%) product. ESI m/z calcd. for C₆₄H₉₉N₈O₂₆ [M+H]⁺: 1395.7, found: 1395.7.

### Example 387. Synthesis of (50R, 51R)-1-benzyl 57-(perfluorophenyl) 50, 51-bis(((benzyloxy) carbonyl)amino)-17, 33, 49, 52-tetraoxo-4, 7, 10, 13, 20, 23, 26, 29, 36, 39, 42, 45-dodecaoxa-16, 32, 48, 53-tetraazaheptapentacontane-1, 57-dioate (349)

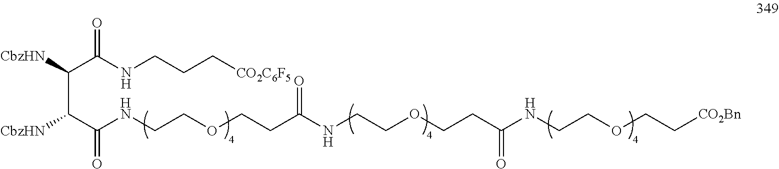

To a solution of compound 348 (0.53 g, 0.40 mmol, 1.0 eq.) in 10 mL DCM, pentafluorophenol (0.081 g, 0.44 mmol, 1.1 eq.) and EDC (0.38 g, 2.0 mmol, 5.0 eq.) were added. The reaction mixture was stirred at r.t. overnight and then washed with cold water (5 mL×2) and brine (10 mL×1), dried over anhydrous sodium sulfate, filtered and concentrated. The crude product was used directly in the next step. ESI m/z calcd. for C₇₀H₉₈ F₅N₆O₂₆ [M+H]⁺: 1561.6, found: 1561.6.

### Example 388. Synthesis of(2S, 4R)-5-(3-((52S, 53S, 64S)-52, 53-bis(((benzyloxy)carbonyl) amino)-3, 19, 35, 51, 54, 59, 62, 65-octaoxo-64-(28-oxo-2, 5, 8, 11, 14, 17, 20, 23, 26-nonaoxa-29-azatritriacontan-33-yl)-1-phenyl-2, 6, 9, 12, 15, 22, 25, 28, 31, 38, 41, 44, 47-tridecaoxa-18, 34, 50, 55, 60, 63, 66-heptaazaheptacontanamido)-4-hydroxyphenyl)-4-((tert-butoxycarbonyl)amino)-2-methylpentanoic acid (350)

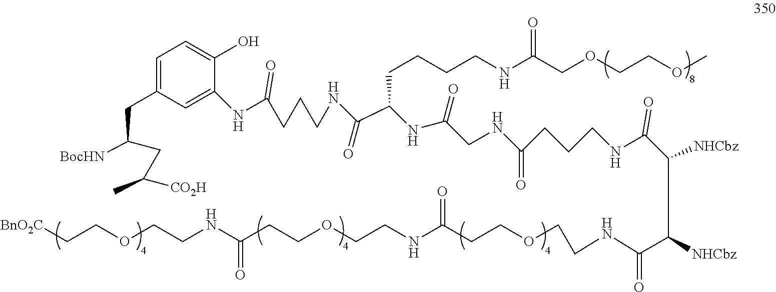

The crude product from the previous step (0.40 mmol, 1.0 eq.) was dissolved in 10 mL DMF, cooled over ice water bath. To which (2S, 4R)-5-(3-((S)-34-(2-aminoacetamido)-28, 35-dioxo-2, 5, 8, 11, 14, 17, 20, 23, 26-nonaoxa-29, 36-diazatetracontanamido)-4-hydroxyphenyl)-4-((tert-butoxycarbonyl)amino)-2-methylpentanoic acid (0.420 g, 0.406 mmol) and DIPEA (0.15 g, 1.2 mmol) were added in sequence. After stirring over the ice bath for 1 hour, the reaction was concentrated, and re-dissolved in a small amount of DCM, loaded on a silica gel column and eluted with 0-20% MeOH/DCM to give a colorless oil (0.531 g, 56% yield). ESI m/z calcd. for C₁₁₂H₁₇₉N₁₂O₄₁ [M+H]⁺: 2348.2291, found: 2348.2380.

### Example 389. Synthesis of (2S, 4R)-4-((tert-butoxycarbonyl)amino)-5-(3-((52S, 53S, 64S)-52, 53-diamino-3, 19, 35, 51, 54, 59, 62, 65-octaoxo-64-(28-oxo-2, 5, 8, 11, 14, 17, 20, 23, 26-nonaoxa-29-azatritriacontan-33-yl)-1-phenyl-2, 6, 9, 12, 15, 22, 25, 28, 31, 38, 41, 44, 47-tridecaoxa-18, 34, 50, 55, 60, 63, 66-heptaazaheptacontanamido)-4-hydroxyphenyl)-2-methylpentanoic acid (351)

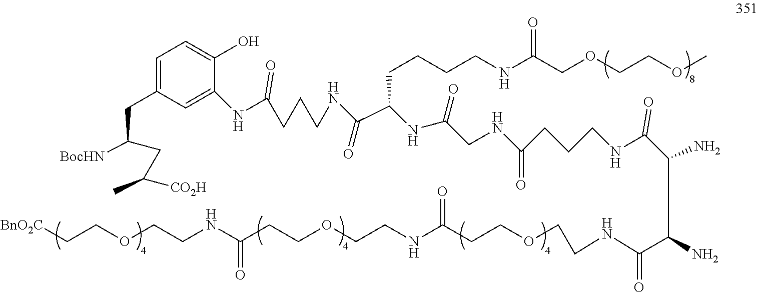

A mixture of compound 350 (0.53 g, 0.22 mmol) and dry palladium carbon (0.1 g, 10% wt) in 10 mL methanol was stirred under a H₂ balloon at r.t. overnight. The reaction mixture was filtered and the filtrate was evaporated to give 0.35 g (yield 76%) of crude material, which was directly used for the next reaction. ESI m/z calcd. for C₉₆H₁₆₇N₁₂O₃₇ [M+H]⁺: 2080.1556, found: 2080.1645.

### Example 390. Synthesis of (2S, 4R)-5-(3-((52S, 53S, 64S)-52, 53-bis(3-(2, 5-dioxo-2, 5-dihydro-1H-pyrrol-1-yl)propanamido)-3, 19, 35, 51, 54, 59, 62, 65-octaoxo-64-(28-oxo-2, 5, 8, 11, 14, 17, 20, 23, 26-nonaoxa-29-azatritriacontan-33-yl)-1-phenyl-2, 6, 9, 12, 15, 22, 25, 28, 31, 38, 41, 44, 47-tridecaoxa-18, 34, 50, 55, 60, 63, 66-heptaazaheptacontanamido)-4-hydroxyphenyl)-4-((tert-butoxycarbonyl)amino)-2-methylpentanoic acid (352)

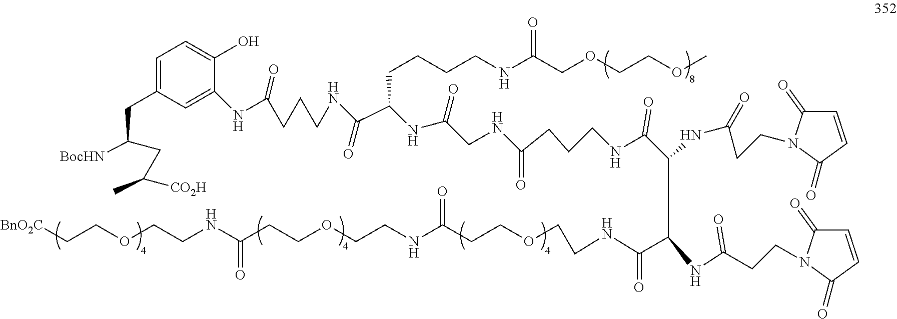

To the crude product from the previous step (0.350 g, 0.168 mmol) dissolved in the mixture of 5 mL of ethanol and 0.5 mL of 0.1M NaH₂PO₄, N-succinimidyl (3-maleimido)propanoate (0.200 g, 0.751 mmol) was added. The reaction mixture was stirred at r.t. overnight, and then concentrated. The residue was dissolved in a small amount of water, and loaded on C-18 gel column, eluted with 100-20% water/MeOH, pooled the fractions containing the product, concentrated and lyophilized to give a colorless oil product (0.23 g, 57% yield). ESI m/z calcd. for C₁₁₀H₁₇₇N₁₄O₄₃ [M+H]⁺: 2382, 2095, found:2382.2190.

### Example 391. Synthesis of (34S, 455, 46S)-34-((4-((5-((2S, 4S)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxamido)-4-carboxypentyl)-2-hydroxyphenyl)amino)-4-oxobutyl)carbamoyl)-45, 46-bis(3-(2, 5-dioxo-2, 5-dihydro-1H-pyrrol-1-yl)propanamido)-28, 36, 39, 44, 47, 63, 79-heptaoxo-2, 5, 8, 11, 14, 17, 20, 23, 26, 51, 54, 57, 60, 67, 70, 73, 76, 83, 86, 89, 92-henicosaoxa-29, 35, 38, 43, 48, 64, 80-heptaazapentanonacontan-95-oic acid (353)

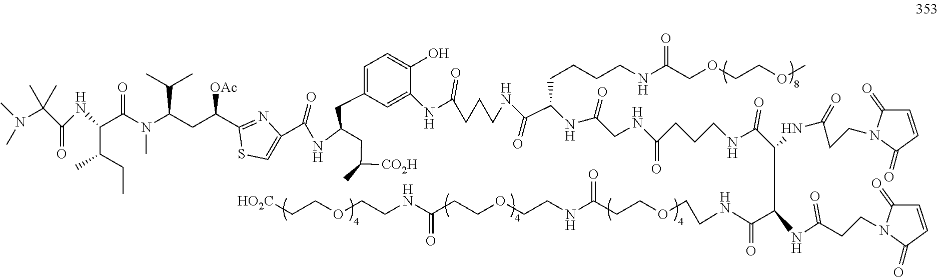

Compound 352 (0.131 g, 0.0546 mmol) was dissolved in 2 mL of DCM, and stirred with 2 mL of TFA at r.t. for 3 h. The solvents were removed and the residue was co-evaporated with DCM for three times, placed on high vacuum pump.

The crude product was re-dissolved in DMF (1.2 mL) and cooled over an ice water bath. Perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (Tub-pentafluorophenol) (0.048 g, 0.0690 mmol) was added, followed by addition of DIPEA (0.10 g). The reaction was stirred over the ice bath for 1 hour and then adjusted to pH 4-5 using formic acid. The mixture was concentrated, re-dissolved in a small amount of DCM, and loaded onto a silica gel column, and eluted with MeOH/DCM (1:10 to 1:4, all containing 0.1% formic acid). Fractions containing the product were combined and concentrated to give 0.112 g of yellow foam (75% yield in two steps). The product was further purified by preparative HPLC (15-50% MeCN/H₂O containing 0.1% formic acid). Fractions were combined and concentrated to give a colorless oil (0.084 g, 57% yield). ESI m/z calcd. for C₁₂₃H₂₀₃N₁₈O₄₆S [M+H]⁺: 2700.3820, found: 2700.3925.

### Example 392. Synthesis of 2, 5, 8, 11, 14, 17, 20, 23-octaoxapentacosan-25-yl 4-methylbenzenesulfonate (354)

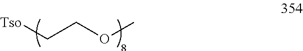

To a solution of mPEG₈-OH (10 g, 26 mmol, 1.0 eq.) in 100 mL of anhydrous DCM, TEA (10.5 g, 104 mmol, 4.0 eq.), DMAP (32 mg, 0.26 mmol, 0.01 eq.) and TsCl (14.9 g, 78 mmol, 3.0 eq.) were added in sequence over an ice water bath. The reaction was stirred at 0° C. for 10 min, then warmed to r.t. and stirred overnight. The reaction was washed with 1N HCl washing (100 mL×1), water (100 mL×1) and brine washing (100 mL×1), dried over anhydrous sodium sulfate, filtered and concentrated. The residue was dissolved in a small amount of DCM and loaded onto a silica gel column, eluted with EA/PE (5-100%) and 1-3% MeOH/DCM. Fractions were combined and concentrated to give a yellow oil (11.6 g, 83% yield). ESI m/z calcd. for C₂₄H₄₃O₁₁S [M+H]⁺: 539.2, found:539.2.

### Example 393. Synthesis of N, N-dibenzyl-2, 5, 8, 11, 14, 17, 20, 23-octaoxapentacosan-25-amine (355)

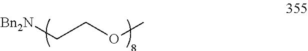

A mixture of 2, 5, 8, 11, 14, 17, 20, 23-octaoxapentacosan-25-yl 4-methylbenzene sulfonate (11.6 g, 21.5 mmol, 1.0 eq.) and dibenzylamine (5.5 g, 27.8 mmol, 1.5 eq.) in 20 mL of anhydrous DMF was heated to 100° C. with stirring overnight. The reaction was diluted with 300 mL of DCM, washed with water (300 mL×3) and brine (300 mL×1), dried over anhydrous sodium sulfate, filtered and concentrated. The residue was purified on silica gel column (50-100% EA/PE) to give a light yellow oil (8.2 g, 66% yield). ESI m/z calcd. for C₃₁H₅₀NO₈ [M+H]⁺: 564.3, found:564.3.

### Example 394. Synthesis of 2, 5, 8, 11, 14, 17, 20, 23-octaoxapentacosan-25-amine (356)

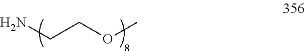

A solution mixture of N, N-dibenzyl-2, 5, 8, 11, 14, 17, 20, 23-octaoxapentacosan-25-amine (8.6 g, 15.2 mmol) and dry palladium on carbon (0.9 g, 10 wt %) in 100 mL of anhydrous methanol was refluxed under a H₂ balloon overnight. The catalyst was filtered off and washed with methanol. The filtrate was evaporated to give 5.3 g of colorless oil (yield 90%). ESI m/z calcd. for C₁₇H₃₈NO₈ [M+H]⁺: 384.3, found:384.3.

### Example 395. Synthesis of (28R, 29R)-tert-butyl 28, 29-bis(((benzyloxy)carbonyl) amino)-27, 30-dioxo-2, 5, 8, 11, 14, 17, 20, 23-octaoxa-26, 31-diazapentatriacontan-35-oate (357)

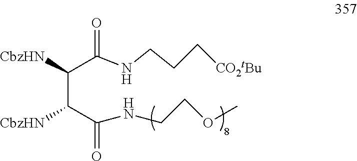

Compound 340 (1.6 g, 2.84 mmol, 1.0 eq.) and 2, 5, 8, 11, 14, 17, 20, 23-octaoxa pentacosan-25-amine (1.2 g, 2.84 mmol, 1.0 eq.) were dissolved in 5 mL of anhydrous DMF, to which HATU (3.2 g, 8.52 mmol, 3.0 eq.) and DIPEA (1.5 g, 11.36 mmol, 4.0 eq.) were added in sequence over an ice water bath. The reaction was stirred over the bath for 2 h, then 150 mL of water was added, and extracted with DCM (150 mL×1, 100 mL×1). The organic phase was washed with 1 N HCl (200 mL×1), saturated sodium bicarbonate (200 mL×1) and brine (200 mL×1), dried over anhydrous sodium sulfate, filtered and concentrated. The crude product was dissolved in a small amount of DCM and loaded on a silica gel column, and then eluted with 0-5% MeOH/DCM. Fractions were combined and concentrated to give 2.29 g of white solid (87% yield). ESI m/z calcd. for C₄₅H₇₁N₄O₁₆ [M+H]⁺: 923.5, found:923.5.

### Example 396. Synthesis of (28R, 29R)-28, 29-bis(((benzyloxy)carbonyl)amino)-27, 30-dioxo-2, 5, 8, 11, 14, 17, 20, 23-octaoxa-26, 31-diazapentatriacontan-35-oic acid (358)

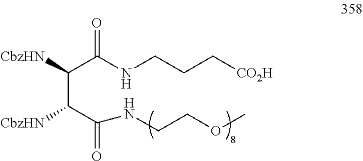

A solution of compound 357 (2.29 g, 2.48 mmol) in the mixture of 5 mL of DCM, and 5 mL of TFA was stirred at r.t. for 3 h. The solvents were removed and the residue was co-evaporated with DCM for three times, the residue was dissolved in a small amount of DCM, and loaded on a silica gel column, eluted with 5-8% MeOH/DCM. Fractions were combined and concentrated to give 2.09 g of white jelly solid (97% yield). ESI m/z calcd. for C₄₁H₆₃N₄O₁₆ [M+H]⁺: 867.4, found:867.4.

### Example 397. Synthesis of (28R, 29R)-perfluorophenyl 28, 29-bis(((benzyloxy)carbonyl) amino)-27, 30-dioxo-2, 5, 8, 11, 14, 17, 20, 23-octaoxa-26, 31-diazapentatriacontan-35-oate (359)

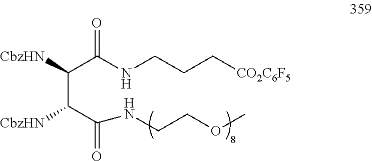

To a solution of compound 358 (1.5 g, 1.73 mmol, 1.0 eq.) in 10 mL of DCM over an ice water bath, pentafluorophenol (0.35 g, 1.90 mmol, 1.1 eq.) and EDC (1.7 g, 8.66 mmol, 5.0 eq.) were added. The reaction was warmed to r.t. and stirred for 5 h, then washed with water (10 mL×2) and brine (20 mL×1), dried over anhydrous sodium sulfate, filtered and concentrated to give 1.07 g of crude product (60% yield). ESI m/z calcd. for C₄₇H₆₂F₅N₄O₁₆ [M+H]⁺: 1033.4, found:1033.4.

### Example 398. Synthesis of (2S, 4R)-5-(3-((28S, 29S, 43S)-28, 29-bis(((benzyloxy)carbonyl) amino)-27, 30, 35, 38, 41, 44-hexaoxo-43-(28-oxo-2, 5, 8, 11, 14, 17, 20, 23, 26-nonaoxa-29-azatritriacontan-33-yl)-2, 5, 8, 11, 14, 17, 20, 23-octaoxa-26, 31, 36, 39, 42, 45-hexaazanonatetracontanamido)-4-hydroxyphenyl)-4-((tert-butoxycarbonyl)amino)-2-methylpentanoic acid (360)

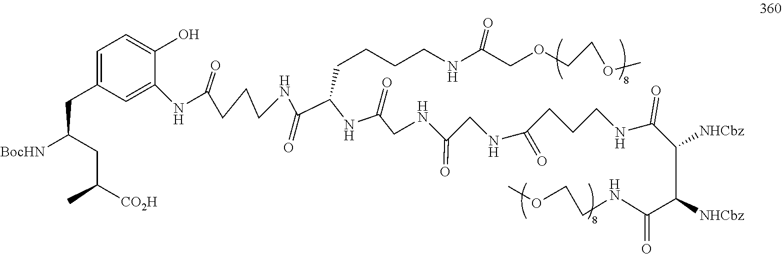

The above crude product (1.07 g, 1.00 mmol) in 10 mL of DMF over an ice water bath, (2S, 4R)-5-(3-((S)-34-(2-(2-aminoacetamido)acetamido)-28, 35-dioxo-2, 5, 8, 11, 14, 17, 20, 23, 26-nonaoxa-29, 36-diazatetracontanamido)-4-hydroxyphenyl)-4-((tert-butoxy carbonyl)amino)-2-methylpentanoic acid (1.091 g, 1.00 mmol) and DIPEA (0.39 g, 3.0 mmol) were added. The reaction was stirred over the bath for 1 h, and adjusted to pH 4-5 using 1N HCl, diluted with EA (100 mL), extracted with water (30 mL×5). The aqueous phase was concentrated and then re-dissolved in a small amount of DCM, loaded a silica gel column and eluted with 15-18% MeOH/DCM. Fractions were combined and concentrated to afford 0.88 g of colorless oil (51% yield). ESI m/z calcd. for C₉₁H₁₄₈N₁₁O₃₄ [M+H]⁺: 1939.0191, found:1939.0280.

### Example 399. Synthesis of (2S, 4R)-4-((tert-butoxycarbonyl)amino)-5-(3-((28S, 29S, 43S)-28, 29-diamino-27, 30, 35, 38, 41, 44-hexaoxo-43-(28-oxo-2, 5, 8, 11, 14, 17, 20, 23, 26-nonaoxa-29-azatritriacontan-33-yl)-2, 5, 8, 11, 14, 17, 20, 23-octaoxa-26, 31, 36, 39, 42, 45-hexaazanonatetracontanamido)-4-hydroxyphenyl)-2-methylpentanoic acid (361)

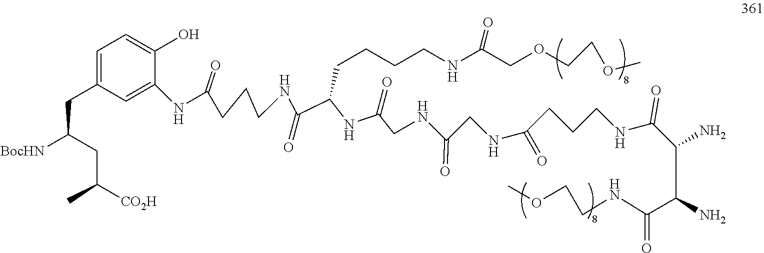

A mixture of previous compound (0.921 g, 0.475 mmol) and palladium on carbon (0.10 g, 10 wt %) in 15 mL of methanol was stirred under a H₂ balloon at r.t. overnight. The catalyst was filtered and filtrated solution was concentrated to give 0.780 g (yield 97%) of crude material, which was directly used for the next reaction. ESI m/z calcd. for C₇₅H₁₃₆N₁₁O₃₀ [M+H]⁺: 1670.9455, found:1670.9560.

### Example 400. Synthesis of (2S, 4R)-5-(3-((28S, 29S, 43S)-28, 29-bis(3-(2, 5-dioxo-2, 5-dihydro-1H-pyrrol-1-yl)propanamido)-27, 30, 35, 38, 41, 44-hexaoxo-43-(28-oxo-2, 5, 8, 11, 14, 17, 20, 23, 26-nonaoxa-29-azatritriacontan-33-yl)-2, 5, 8, 11, 14, 17, 20, 23-octaoxa-26, 31, 36, 39, 42, 45-hexaazanonatetracontanamido)-4-hydroxyphenyl)-4-((tert-butoxycarbonyl)amino)-2-methylpentanoic acid (362)

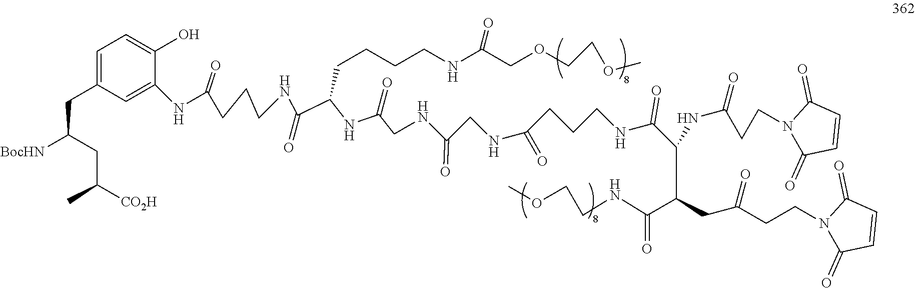

The crude product from the previous step (0.751 g, 0.450 mmol) dissolved in the mixture of 8 mL of ethanol and 1.2 mL of 0.1 M NaH₂PO₄, N-succinimidyl (3-maleimido) propanoate (0.202 g, 0.758 mmol) was added. The reaction mixture was stirred at r.t. overnight, and then concentrated. The residue was dissolved in a small amount of water, and loaded on C-18 gel column, eluted with 0-50% MeOH/water to give a colorless oil (0.603 g, yield 68%). ESI m/z calcd. for C₈₉H₁₄₆N₁₃O₃₆ [M+H]⁺: 1972.9994, found:1973.0090.

### Example 401. Synthesis of (2S, 4S)-5-(3-((28S, 29S, 43S)-28, 29-bis(3-(2, 5-dioxo-2, 5-dihydro-1H-pyrrol-1-yl)propanamido)-27, 30, 35, 38, 41, 44-hexaoxo-43-(28-oxo-2, 5, 8, 11, 14, 17, 20, 23, 26-nonaoxa-29-azatritriacontan-33-yl)-2, 5, 8, 11, 14, 17, 20, 23-octaoxa-26, 31, 36, 39, 42, 45-hexaazanonatetracontanamido)-4-hydroxyphenyl)-4-(2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxamido)-2-methylpentanoic acid (363)

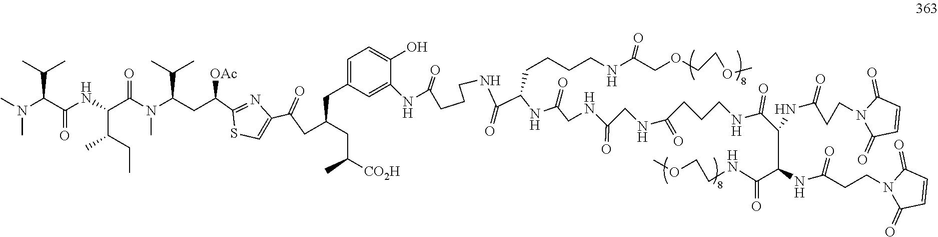

A solution of compound 362 (0.291 g, 0.147 mmol) in 3 mL of DCM and 1 mL of TFA was stirred at r.t. for 0.5 h. The solvents were removed and the residue was co-evaporated with DCM/toluene for three times, placed on high vacuum pump.

The crude product was re-dissolved in 5 mL of DMF and cooled over an ice water bath. Perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (0.121 g, 0.171 mmol) and DIPEA (0.265 g, 2.07 mmol) were added in. The reaction was stirred over the ice bath for 1 hour and then adjusted to pH 4-5 using formic acid. The mixture was concentrated, re-dissolved in a small amount of DCM, and loaded onto a silica gel column, and eluted with MeOH/DCM (1:6 to 1:3, containing 0.1% formic acid). Fractions were combined and concentrated to give 0.213 g of yellow foam product (61% yield in two steps). The product was further purified by preparative C-18 HPLC (25-50% MeCN/H₂O containing 0.1% formic acid). Fractions were combined and concentrated, lyophilized to give a colorless oil product (0.171 g, 48% yield in two steps). ESI m/z calcd. for C₁₁₀H₁₈₀N₁₇O₃₉S [M+H]⁺: 2395.2346, found: 2395.2440.

### Example 402. Synthesis of (6S, 13S)-di-tert-butyl 6, 13-bis(4-aminobutyl)-9, 10-bis(3-(2, 5-dioxo-2, 5-dihydro-1H-pyrrol-1-yl)propanamido)-5, 8, 11, 14-tetraoxo-4, 7, 12, 15-tetraazaoctadecane-1, 18-dioate (364)

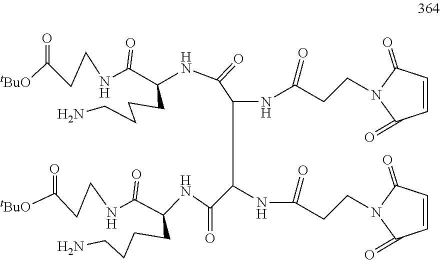 [SMILES: CC(C)(C)OC(=O)CCNC(=O)[C@H](CCCCN)NC(=O)C(NC(=O)CCN1C(=O)C=CC1=O)C(NC(=O)CCN1C(=O)C=CC1=O)C(=O)N[C@@H](CCCCN)C(=O)NCCC(=O)OC(C)(C)C]

To a solution of compound 234 (8.50 g, 6.80 mmol) in methanol (100 mL), NH₄F (0.80 g, 21.62 mmol) and a drop of 1.0 M HCl (˜0.010 mL) were added. The reaction was kept for stirring at r.t for 2 h, following by 50° C. for 2 h. The mixture was then diluted with DMF (30 mL), evaporated in vacuo and dried with oil vacuum pump to give the crude product (8.19 g, >100% yield) for next step without further purification. ESI MS m/z 961.53 ([M+H]t

### Example 403. Synthesis of (6S, 13S)-di-tert-butyl 9, 10-bis(3-(2, 5-dioxo-2, 5-dihydro-1H-pyrrol-1-yl)propanamido)-5, 8, 11, 14-tetraoxo-6, 13-bis(29-oxo-2, 5, 8, 11, 14, 17, 20, 23, 26-nonaoxa-30-azatetratriacontan-34-yl)-4, 7, 12, 15-tetraazaoctadecane-1, 18-dioate (365)

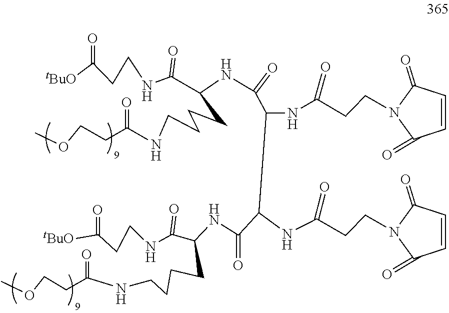

To a solution of the above crude compound (8.19 g,˜6.80 mmol) in D1A(100 mL), 2, 5, 8, 11, 14, 17, 20, 23, 26-nonaoxanonacosan-29-oic acid (6.92 g, 15.17 mmol) and EDC·HCl (6.30 g, 33.15 mmol) were added. The reaction mixture was stirred at r.t. for 8 h, then concentrated, diluted with water (50 mL) and extracted with ethyl acetate (3×80 mL). The combined organic phase was dried over anhydrous sodium sulfate, filtered, concentrated and purified by silica gel column (10%-30% MeOH/DCM) to give a colorless oil (6.51 g, 52% yield in two steps). ESI MS m/z 1839.09 [M+H]⁺.

### Example 404. Synthesis of (6S, 13S)-9, 10-bis(3-(2, 5-dioxo-2, 5-dihydro-1H-pyrrol-1-yl) propanamido)-5, 8, 11, 14-tetraoxo-6, 13-bis(29-oxo-2, 5, 8, 11, 14, 17, 20, 23, 26-nonaoxa-30-azatetratriacontan-34-yl)-4, 7, 12, 15-tetraazaoctadecane-1, 18-dioic acid (366)

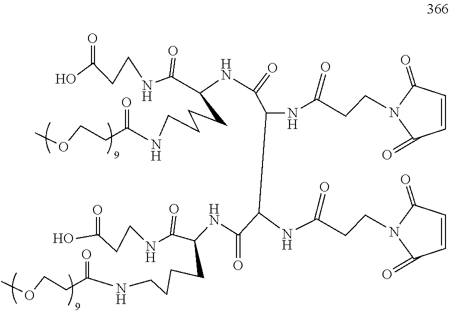

A solution of the above compound (6.49 g, 3.53 mmol) in dioxane (30 mL) was treated with concentrated HCl (10 mL) at 0° C. for 30 min, then diluted with toluene (50 mL), concentrated and purified on a short silica gel column with elution of 10-25% methanol/dichloromethane to give the colorless oil product (5.47 g, 90% yield). ESI MS m/z 1725.88 ([M+H]⁺).

### Example 405. Synthesis of (18S, 25S)-di-tert-butyl 21, 22-bis(3-(2, 5-dioxo-2, 5-dihydro-1H-pyrrol-1-yl)propanamido)-4, 7, 10, 13, 17, 20, 23, 26, 30, 33, 36, 39-dodecaoxo-18, 25-bis(29-oxo-2, 5, 8, 11, 14, 17, 20, 23, 26-nonaoxa-30-azatetratria contan-34-yl)-3, 6, 9, 12, 16, 19, 24, 27, 31, 34, 37, 40-dodecaazadotetracontane-1, 42-dioate (367)

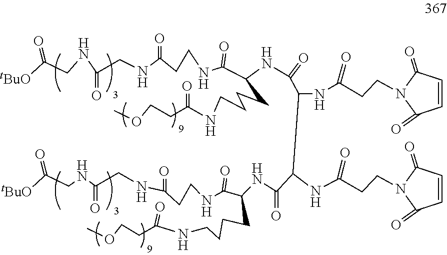

To a solution of compound 366 (5.40 g, 3.13 mmol) in DMA(100 mL), tert-butyl 2-(2-(2-(2-aminoacetamido)acetamido)acetamido)acetate (H-Gly-Gly-Gly-Gly-O^{t}Bu) (2.50 g, 8.27 mmol) and EDC·HCl (5.50 g, 28.94 mmol) were added. The reaction mixture was stirred at r.t. for 8 h, then concentrated, diluted with water (50 mL) and extracted with ethyl acetate (3×80 mL). The combined organic phase was dried over anhydrous sodium sulfate, filtered, concentrated and purified by silica gel column (5%-20% MeOH/DCM) to give the product as a colorless oil (5.95 g, 83% yield). ESI MS m/z 2294.52 ([M+H]⁺).

### Example 406. Synthesis of (18S, 25S)-21, 22-bis(3-(2, 5-dioxo-2, 5-dihydro-1H-pyrrol-1-yl) propanamido)-4, 7, 10, 13, 17, 20, 23, 26, 30, 33, 36, 39-dodecaoxo-18, 25-bis(29-oxo-2, 5, 8, 11, 14, 17, 20, 23, 26-nonaoxa-30-azatetratriacontan-34-yl)-3, 6, 9, 12, 16, 19, 24, 27, 31, 34, 37, 40-dodecaazadotetracontane-1, 42-dioic acid (368)

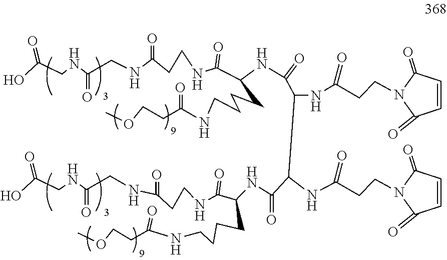

A solution of compound 367 (5.90 g, 2.57 mmol) in dioxane (30 mL) was treated with concentrated HCl (10 mL) at 0° C. for 30 min, then diluted with toluene (50 mL), concentrated and loaded on a short silica gel column and eluted with 10-30% methanol/dichloromethane to give the product as a colorless oil (4.60 g, 82% yield). ESI MS m/z 2182.33 ([M+H]⁺).

### Example 407. Synthesis of (17S, 24S)-bis(2, 5-dioxopyrrolidin-1-yl) 20, 21-bis(3-(2, 5-dioxo-2, 5-dihydro-1H-pyrrol-1-yl)propanamido)-4, 7, 9, 12, 16, 19, 22, 25, 29, 32, 35, 38-dodecaoxo-17, 24-bis(29-oxo-2, 5, 8, 11, 14, 17, 20, 23, 26-nonaoxa-30-azatetratria contan-34-yl)-3, 6, 8, 11, 15, 18, 23, 26, 30, 33, 36, 39-dodecaazahentetracontane-1, 41-dioate (369)

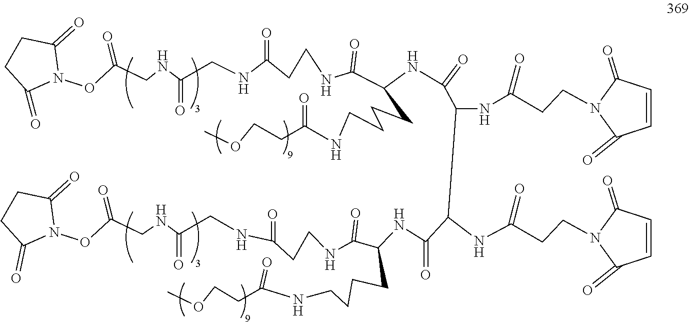

To a mixture of compound 368 (2.30 g, 1.05 mmol) and NHS (0.270 g, 2.34 mmol) in DMF (25 mL) was added EDC (0.785 g, 4.08 mmol). The mixture was stirred for 6 h, concentrated and purified on silica gel column eluted with EtOAc/DCM (1:5-1:1) to afford the title compound (1.88 g, 76% yield). ESI MS m/z 2362.05 ([M+H]⁺).

### Example 408. Synthesis of (2S, 2's, 4R, 4′R)-5, 5′-((((17S, 24S)-20, 21-bis(3-(2, 5-dioxo-2, 5-dihydro-1H-pyrrol-1-yl)propanamido)-4, 7, 9, 12, 16, 19, 22, 25, 29, 32, 35, 38-dodecaoxo-17, 24-bis(29-oxo-2, 5, 8, 11, 14, 17, 20, 23, 26-nonaoxa-30-azatetra triacontan-34-yl)-3, 6, 8, 11, 15, 18, 23, 26, 30, 33, 36, 39-dodecaazahen tetracontane-1, 41-dioyl)bis(azanediyl))bis(4-hydroxy-3, 1-phenylene))bis(4-((tert-butoxycarbonyl) amino)-2-methylpentanoic acid) (370)

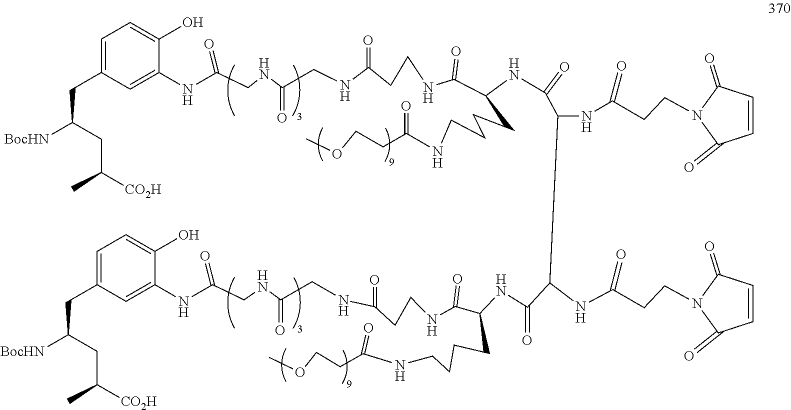

To a mixture of (2S, 4R)-5-(3-amino-4-hydroxyphenyl)-4-((tert-butoxycarbonyl)amino)-2-methylpentanoic acid (201 mg, 0.594 mmol) in DMA (10 mL) and 0.1 M NaH₂PO₄ buffer (pH 7.5, 5 mL) was added compound 369 (712 mg, 0.301 mmol) in four portions in 1 h. The mixture was stirred for another 2 h, concentrated, purified on C-18 HPLC eluted with water/methanol (from 100% water to 50% of water), pooled the fractions containing the product, concentrated, and dried over the vacuum oil pump to afford the title compound (519 mg, 62% yield). MS-ESI m/z: [M+H]⁺ calcd. for C₁₂₅H₁₉₉N₂₂O₅₀, 2808.3706; found, 2808.3790.

### Example 409. Synthesis of 371

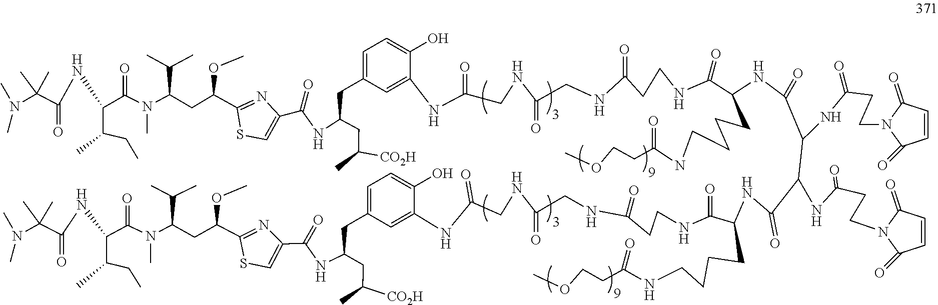

To a solution of compound 370 (251 mg, 0.0894 mmol) dissolved in 2 mL of dioxane was added HCl (conc. 0.5 mL). The mixture was stirred at r.t. for 45 min, diluted with toluene, concentrated and the residue was co-evaporated with DMF (5 mL) for three times, placed on high vacuum pump.

The crude product was re-dissolved in DMF (4 mL) and cooled over an ice water bath. Perfluorophenyl 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylate (145 mg, 0.218 mmol) was added, followed by addition of DIPEA (0.4 mL). The reaction was the stirred at r.t. for 1 hour, then concentrated, diluted with DMF (4 mL), adjusted to pH 4-5 using formic acid and purified by preparative C-18 HPLC (15-50% MeCN/H₂O containing 0.1% formic acid). Fractions were combined, concentrated, lyophilized to give a colorless foam (193 mg, 61% yield in two steps) of the product. ESI m/z calcd. for C₁₆₃H₂₆₃N₃₀O₅₄S₂[M+H]⁺: 3568.8198, found: 3588.8320.

### Example 410. Synthesis of (2S, 5S, 8S, 9S, 12S, 15S)-di-tert-butyl 8, 9-bis(((benzyloxy) carbonyl)amino)-2, 5, 12, 15-tetramethyl-4, 7, 10, 13-tetraoxo-3, 6, 11, 14-tetraazahexadecane-1, 16-dioate (372)

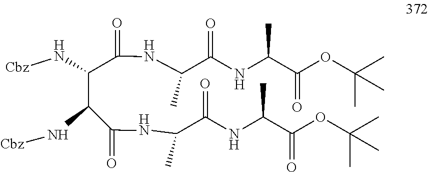 [SMILES: C[C@H](NC(=O)[C@@H](NC(=O)OCc1ccccc1)[C@H](NC(=O)OCc1ccccc1)C(=O)N[C@@H](C)C(=O)N[C@@H](C)C(=O)OC(C)(C)C)C(=O)N[C@@H](C)C(=O)OC(C)(C)C]

To a solution of (S)-tert-butyl 2-((S)-2-aminopropanamido)propanoate (2.00 g, 5.0 mmol) in DMF (30 mL) at about 0° C., dibenzyl ((3S, 4S)-2, 5-dioxotetrahydrofuran-3, 4-diyl)dicarbamate (127, 2.16 g, 10.0 mmol) was added. The mixture was stirred at 0° C. for 30 min, room temperature for 45 min, then re-cooled to about 0° C., followed by addition of DIPEA (640 mg, 5.0 mmol) and EDC (5.21 g, 27.1 mmol). The reaction mixture was warmed to room temperature and stirred for 1 hour, then diluted with dichloromethane (350 mL), washed with saturated NaHCO₃ (150 mL), water (100 mL), dried over anhydrous sodium sulfate, filtered and concentrated. The residue was purified by column chromatography (100:0 to 10:1 dichloromethane/MeOH) to give the title compound (2.71 g, 67% yield). MS-ESI m/z: [M+H]⁺ calcd. for C₄₀H₅₆N₆O₁₂, 813.40; found, 813.40.

### Example 411. Synthesis of (2S, 5S, 8S, 9S, 12S, 15S)-di-tert-butyl 8, 9-diamino-2, 5, 12, 15-tetramethyl-4, 7, 10, 13-tetraoxo-3, 6, 11, 14-tetraazahexadecane-1, 16-dioate (373)

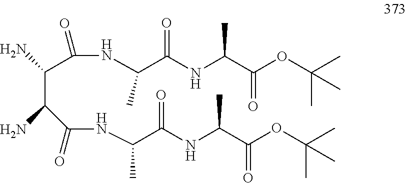 [SMILES: C[C@H](NC(=O)[C@@H](N)[C@H](N)C(=O)N[C@@H](C)C(=O)N[C@@H](C)C(=O)OC(C)(C)C)C(=O)N[C@@H](C)C(=O)OC(C)(C)C]

A mixture of compound 372 (2.65 g, 3.21 mmol) and 10% palladium carbon (100 mg) in methanol (60 mL) was stirred under hydrogen overnight. The solid was filtered off and filtrate concentrated to give a colorless oil (1.762 g, 100% yield). ESI m/z: [M+H]⁺ calcd. for C₂₄H₄₄N₆O₈, 545.32; found, 545.32.

### Example 412. Synthesis of (2S, 5S, 8S, 9S, 12S, 15S)-di-tert-butyl 8, 9-bis(2-(2, 5-dioxo-2, 5-dihydro-1H-pyrrol-1-yl)acetamido)-2, 5, 12, 15-tetramethyl-4, 7, 10, 13-tetraoxo-3, 6, 11, 14-tetraazahexadecane-1, 16-dioate (374)

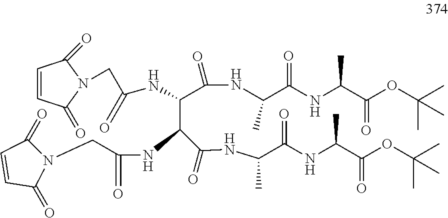 [SMILES: C[C@H](NC(=O)[C@@H](NC(=O)CN1C(=O)C=CC1=O)[C@H](NC(=O)CN1C(=O)C=CC1=O)C(=O)N[C@@H](C)C(=O)N[C@@H](C)C(=O)OC(C)(C)C)C(=O)N[C@@H](C)C(=O)OC(C)(C)C]

To a solution of compound 373 (1.76 g, 3.23 mmol) in DMF (50 mL) was added 2-(2, 5-dioxo-2, 5-dihydro-1H-pyrrol-1-yl)acetic acid (1.25 g, 8.06 mmol) and EDC (2.82 g, 14.68 mmol). The reaction mixture was stirred overnight, concentrated and purified on a silica gel column (dichloromethane/MeOH=100:0 to 10:1) to afford the title compound (2.21 g, 78% yield). MS-ESI m/z: [M+H]⁺ calcd. for C₄₀H₅₈N₈O₁₄, 875.41; found 875.41.

### Example 413. Synthesis of (2S, 5S, 8S, 9S, 12S, 15S)-8, 9-bis(2-(2, 5-dioxo-2, 5-dihydro-1H-pyrrol-1-yl)acetamido)-2, 5, 12, 15-tetramethyl-4, 7, 10, 13-tetraoxo-3, 6, 11, 14-tetraazahexadecane-1, 16-dioic acid (375)

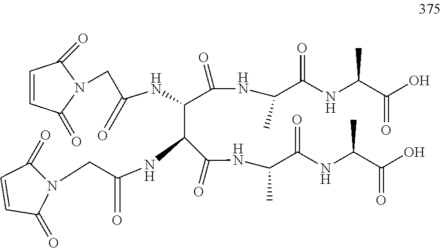 [SMILES: C[C@H](NC(=O)[C@H](C)NC(=O)[C@@H](NC(=O)CN1C(=O)C=CC1=O)[C@H](NC(=O)CN1C(=O)C=CC1=O)C(=O)N[C@@H](C)C(=O)N[C@@H](C)C(=O)O)C(=O)O]

Compound 374 (1.21 g, 1.38 mmol) in dioxane (20 mL) was treated with conc. hydrochloric acid (5 mL) for 0.5 hours. The reaction mixture was diluted with toluene (10 mL), evaporated and dried over oil vacuum pump to give the title product (1.05 g, 100% yield) which was used for the next step without further purification. MS-ESI m/z: [M+H]⁺ calcd. for C₃₂H₄₂N₈O₁₄, 763.28; found, 763.28.

### Example 414. Synthesis of (2S, 2's, 4R, 4′R)-di-tert-butyl 5, 5′-((((7S, 10S, 13S, 16S, 17S, 20S, 23S, 26S)-16, 17-bis(2-(2, 5-dioxo-2, 5-dihydro-1H-pyrrol-1-yl)acetamido)-10, 13, 20, 23-tetramethyl-6, 9, 12, 15, 18, 21, 24, 27-octaoxo-7, 26-bis(28-oxo-2, 5, 8, 11, 14, 17, 20, 23, 26-nonaoxa-29-azatritriacontan-33-yl)-5, 8, 11, 14, 19, 22, 25, 28-octaazadotriacontane-1, 32-dioyl)bis(azanediyl))bis(4-hydroxy-3, 1-phenylene))bis(4-((tert-butoxycarbonyl)amino)-2-methylpentanoate) (376)

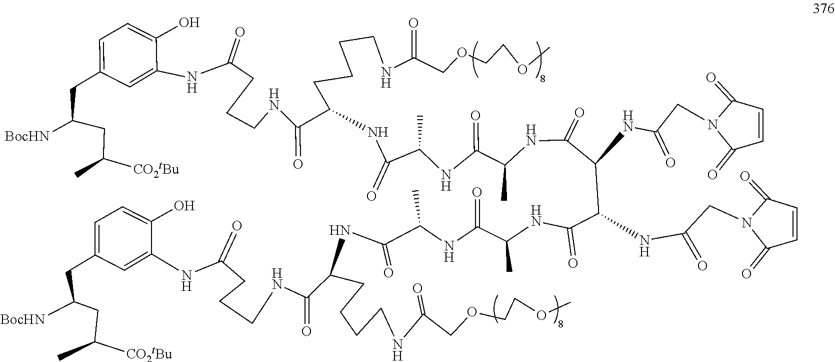

To the above step compound 375 (1.001 g, 1.31 mmol) in THF (50 mL) were added (2S, 4R)-tert-butyl 5-(3-((S)-34-amino-28, 35-dioxo-2, 5, 8, 11, 14, 17, 20, 23, 26-nonaoxa-29, 36-diazatetracontanamido)-4-hydroxyphenyl)-4-((tert-butoxycarbonyl)amino)-2-methylpentanoate (2.801 g, 2.650 mmol), bromo-tris-pyrrolidino-phosphonium hexafluorophosphate (PyBroP) (1.842 g, 3.951 mmol) and DMAP (0.311 g, 2.536 mmol). The mixture was stirred overnight, evaporated, purified on silica gel column eluted with MeOH/DCM (1:10) to afford the title compound (2.613 g, 73% yield). MS-ESI m/z: [M+H]⁺ calcd. for C₁₂₈H₂₀₉N₁₈O₄₆, 2734.4569; found 2734.4675.

### Example 415. Synthesis of (2S, 2's, 4R, 4′R)-5, 5′-((((7S, 10S, 13S, 16S, 17S, 20S, 23S, 26S)-16, 17-bis(2-(2, 5-dioxo-2, 5-dihydro-1H-pyrrol-1-yl)acetamido)-10, 13, 20, 23-tetramethyl-6, 9, 12, 15, 18, 21, 24, 27-octaoxo-7, 26-bis(28-oxo-2, 5, 8, 11, 14, 17, 20, 23, 26-nonaoxa-29-azatritriacontan-33-yl)-5, 8, 11, 14, 19, 22, 25, 28-octaazadotriacontane-1, 32-dioyl)bis(azanediyl))bis(4-hydroxy-3, 1-phenylene))bis(4-amino-2-methylpentanoic acid), HCl salt (377)

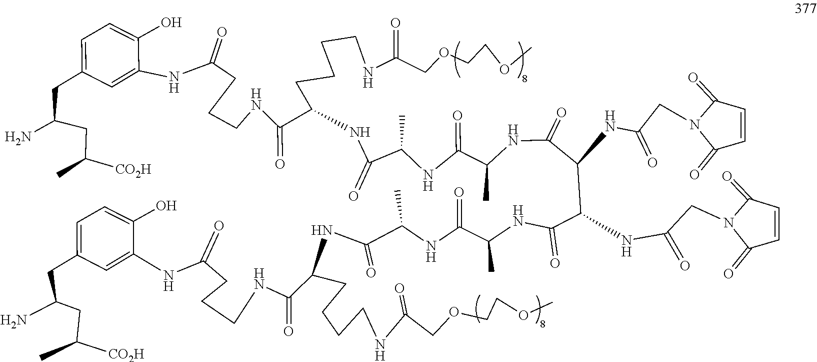

Compound 376 (2.610 g, 0.954 mmol) in dioxane (20 mL) was treated with hydrochloric acid (conc. 5 mL) for 0.5 hours. The reaction mixture was diluted with toluene (10 mL), evaporated and dried over oil vacuum pump to give the title product (2.315 g, 100% yield) which was used for the next step without further purification. MS-ESI m/z: [M+H]⁺ calcd. for C₁₁₀H₁₇₇N₁₈O₄₂, 2422.2269; found, 2422.2375.

### Example 416. Synthesis of 378

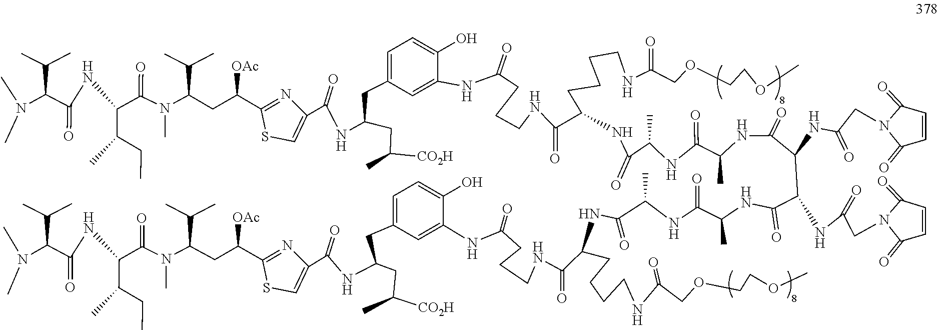

To a solution of compound 377 (0.521 g, 0.215 mmol) dissolved in DMF (8 mL) and cooled over an ice water bath was added perfluorophenyl 2-((3S, 6S, 9R, 11R)-6-((S)-sec-butyl)-3, 9-diisopropyl-2, 8-dimethyl-4, 7, 13-trioxo-12-oxa-2, 5, 8-triazatetradecan-11-yl)thiazole-4-carboxylate (0.362 g, 0.515 mmol), followed by addition of DIPEA (0.50 mL). The reaction was then stirred at r.t. for 1 hour and then adjusted to pH 4-5 using formic acid. The mixture was concentrated, re-dissolved in a small amount of water, and purified by preparative C-18 HPLC (10-60% MeCN/H₂O containing 0.1% formic acid). Fractions were combined, concentrated and lyophilized to give a colorless foam (462 mg, 62% yield). ESI m/z calcd. for C₁₆₂H₂₆₁N₂₆O₅₂S₂[M+H]⁺: 3466.7979, found: 3466.8070.

### Example 417. Synthesis of 379, 380 and 381

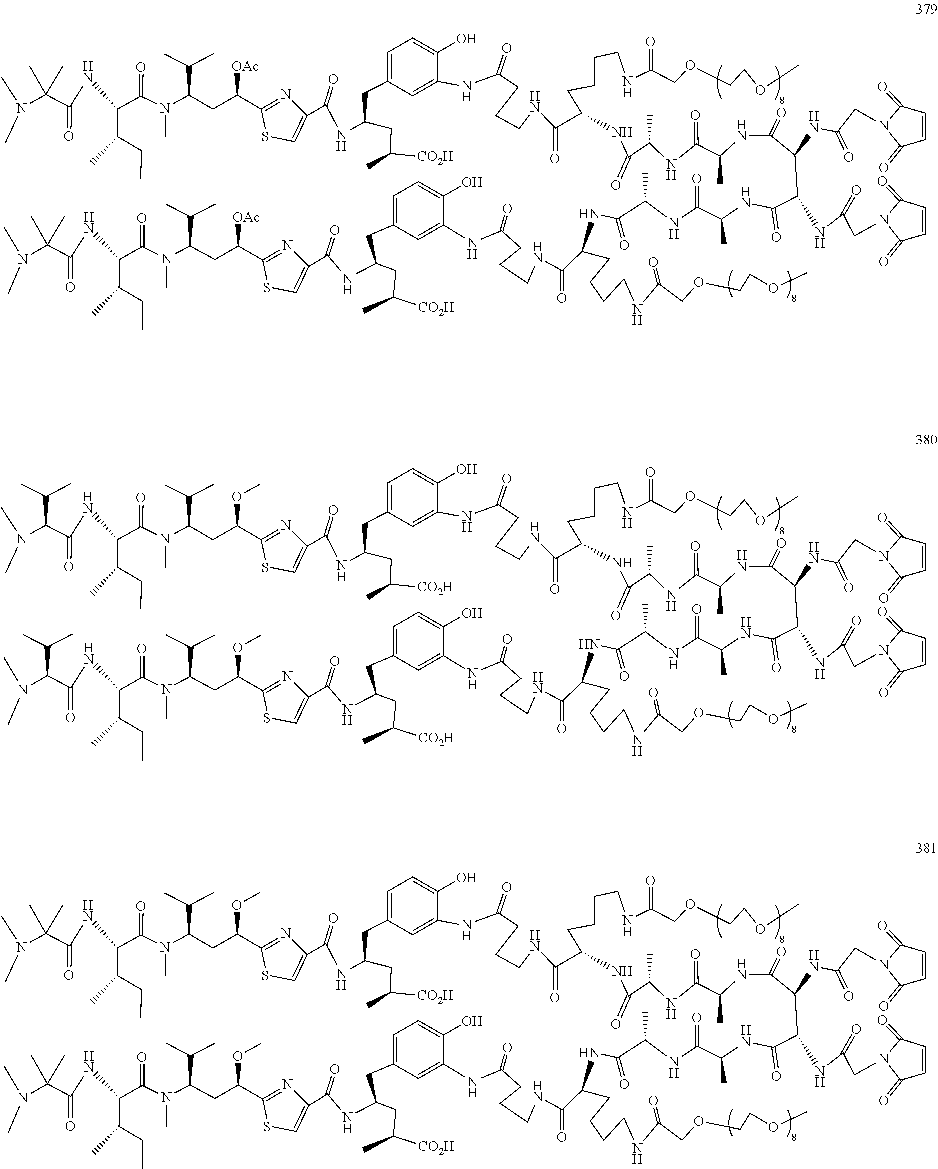

To a solution of compound 377 (200 mg, 0.0825 mmol) dissolved in DMF (5 mL) and cooled over an ice water bath was added perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (0.20 mmol), or perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (0.20 mmol), or perfluorophenyl 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylate (0.20 mmL), followed by addition of DIPEA (0.40 mL). The reaction was then stirred at r.t. for 1 hour and then adjusted to pH 4-5 using formic acid. The mixture was concentrated, re-dissolved in a small amount of water, and purified by preparative C-18 HPLC (10-60% MeCN/H₂O containing 0.1% formic acid). Fractions were combined, concentrated and lyophilized to give a colorless foam (60-65% yield).

379 (175 mg, 62% yield), ESI m/z calcd. for C₁₆₀H₂₅₇N₂₆O₅₂S₂[M+H]⁺: 3438.7707, found: 3438.7830.

380 (168 mg, 60% yield), ESI m/z calcd. for C₁₆₀H₂₆₁N₂₆O₅₀S₂ [M+H]⁺: 3410.8122, found: 3410.8245. 381 (162 mg, 58% yield), ESI m/z calcd. for C₁₅₈H₂₅₇N₂₆O₅₀S₂ [M+H]⁺: 3382.7809, found: 3382.7940.

### Example 418. Synthesis of (2S, 2's, 4R, 4′R)-5, 5′-((((7S, 32S)-19, 20-bis(2-(2, 5-dioxo-2, 5-dihydro-1H-pyrrol-1-yl)acetyl)-6, 9, 12, 17, 22, 27, 30, 33-octaoxo-7, 32-bis(28-oxo-2, 5, 8, 11, 14, 17, 20, 23, 26-nonaoxa-29-azatritriacontan-33-yl)-5, 8, 11, 16, 19, 20, 23, 28, 31, 34-decaazaoctatriacontane-1, 38-dioyl)bis(azanediyl))bis(4-hydroxy-3, 1-phenylene))bis(4-((tert-butoxycarbonyl)amino)-2-methylpentanoic acid) (382)

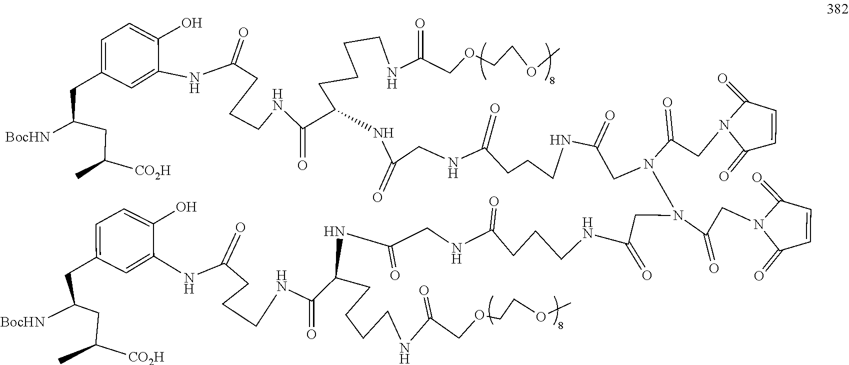

To a mixture of (2S, 4R)-5-(3-((S)-34-(2-aminoacetamido)-28, 35-dioxo-2, 5, 8, 11, 14, 17, 20, 23, 26-nonaoxa-29, 36-diazatetracontanamido)-4-hydroxyphenyl)-4-((tert-butoxycarbonyl)amino)-2-methylpentanoic acid (852 mg, 0.825 mmol) in THF (30 mL) and 0.1 M NaH₂PO₄ buffer (20 mL, pH 7.5) was added bis(2, 5-dioxopyrrolidin-1-yl)4, 4′-((2, 2′-(1, 2-bis(2-(2, 5-dioxo-2, 5-dihydro-1H-pyrrol-1-yl) acetyl)hydrazine-1, 2-diyl)bis(acetyl))bis(azanediyl))dibutanoate (322 mg, 0.409 mmol). The mixture was stirred overnight, evaporated, purified on C-18 HPLC (250 mm (L)×50 mm(d)) eluted with MeOH/water (v=40 mL/min, from 5% to 50% of MeOH in 45 min), pooled the fraction containing the product, concentrated and lyophilzed to afford the title compound (568 mg, 53% yield). MS-ESI m/z: [M+H]⁺ calcd. for C₁₂₀H₁₉₃N₁₈O₄₆, 2622.3317; found 2622.3420.

### Example 419. Synthesis of (2S, 2's, 4R, 4′R)-5, 5′-((((7S, 32S)-19, 20-bis(2-(2, 5-dioxo-2, 5-dihydro-1H-pyrrol-1-yl)acetyl)-6, 9, 12, 17, 22, 27, 30, 33-octaoxo-7, 32-bis(28-oxo-2, 5, 8, 11, 14, 17, 20, 23, 26-nonaoxa-29-azatritriacontan-33-yl)-5, 8, 11, 16, 19, 20, 23, 28, 31, 34-decaazaoctatriacontane-1, 38-dioyl)bis(azanediyl))bis(4-hydroxy-3, 1-phenylene))bis(4-amino-2-methylpentanoic acid), HCl salt To a solution of compound (383)

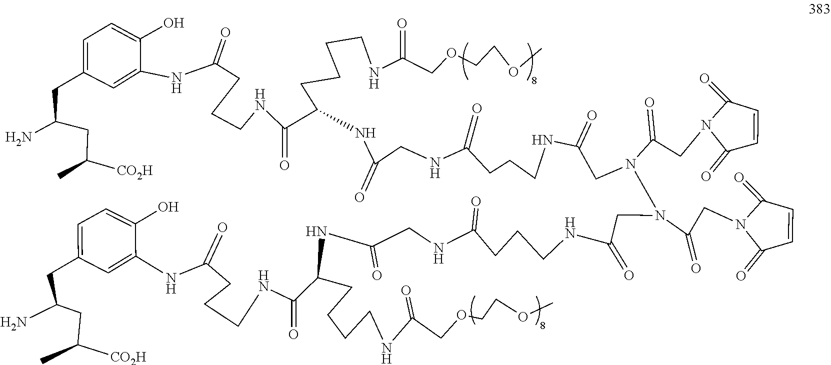

Compound 382 (561 mg, 0.214 mmol) in dioxane (6 mL) was treated with conc. hydrochloric acid (2 mL) for 0.5 hours. The reaction mixture was diluted with toluene (10 mL), evaporated and dried over oil vacuum pump to give the product (520 mg, 101% yield) which was used for the next step without further purification. MS-ESI m/z: [M+H]⁺ calcd. for C₁₁₀H₁₇₇N₁₈O₄₂, 2422.2269; found, 2422.2380.

### Example 420. Synthesis of 384

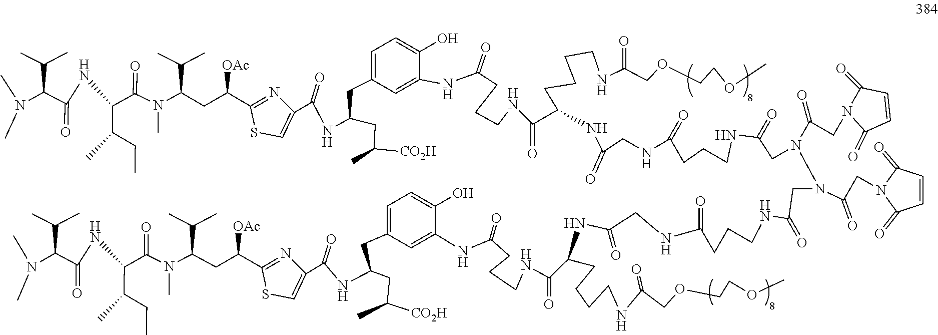

To a solution of compound 383 (0.121 g, 0.050 mmol) dissolved in DMF (3 mL) and cooled over 10 an ice water bath was added perfluorophenyl 2-((3S, 6S, 9R, 11R)-6-((S)-sec-butyl)-3, 9-diiso-propyl-2, 8-dimethyl-4, 7, 13-trioxo-12-oxa-2, 5, 8-triazatetradecan-11-yl)thiazole-4-carboxylate (80.1 mg, 0.113 mmol), followed by addition of DIPEA (0.15 mL). The reaction was then stirred at r.t. for 2 hours and then adjusted to pH 4-5 using formic acid. The mixture was concentrated, re-dissolved in a small amount of water, and purified by preparative C-18 HPLC (10-60%0 MeCN/H₂O containing 0.1% formic acid). Fractions containing the product were combined, concentrated and lyophilized to give a colorless foam (109.1 mg, 63% yield). ESI m/z calcd. for C₁₆₂H₂₆₁N₂₆O₅₂S₂[M+H]⁺: 3466.8020, found: 3466.8130.

### Example 421. Synthesis of 385

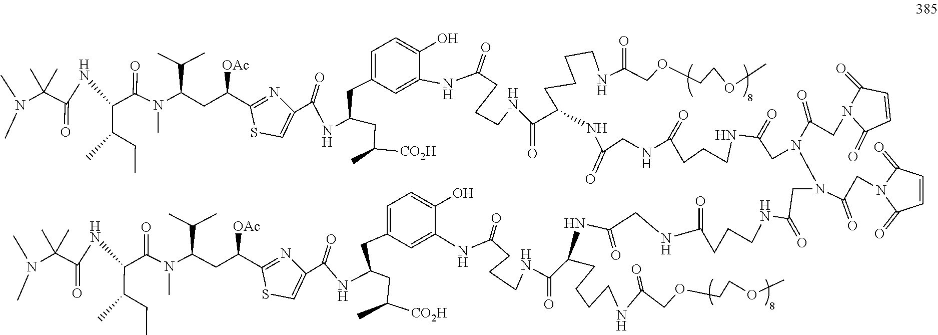

To a solution of compound 383 (0.121 g, 0.050 mmol) dissolved in DMF (3 mL) and cooled over an ice water bath was added perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (83.1 mg, 0.120 mmol), followed by addition of DIPEA (0.15 mL). The reaction was then stirred at r.t. for 2 hours and then adjusted to pH 4-5 using formic acid. The mixture was concentrated, re-dissolved in a small amount of water, and purified by preparative C-18 HPLC (10-60% MeCN/H₂O containing 0.1% formic acid). Fractions containing the product were combined, concentrated and lyophilized to give a colorless foam (104.7 mg, 61% yield). ESI m/z calcd. for C₁₆₀H₂₅₇N₂₆O₅₂S₂[M+H]⁺: 3438.7707, found: 3438.7840.

### Example 422. Synthesis of 386

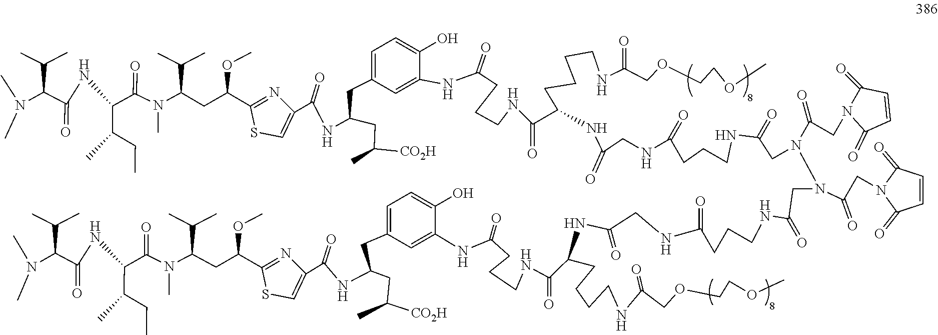

To a solution of compound 383 (121 mg, 0.050 mmol) dissolved in DMF (3 mL) and cooled over an ice water bath was added perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (82.0 mg, 0.121 mmol), followed by addition of DIPEA (0.15 mL). The reaction was then stirred at r.t. for 2 hours and then adjusted to pH 4-5 using formic acid. The mixture was concentrated, re-dissolved in a small amount of water, and purified by preparative C-18 HPLC (10-60% MeCN/H₂O containing 0.1% formic acid). Fractions containing the product were combined, concentrated and lyophilized to give a colorless foam (110.2 mg, 65% yield). ESI m/z calcd. for C₁₆₀H₂₆₁N₂₆O₅₀S₂ [M+H]⁺: 3410.8122, found: 3410.8240.

### Example 423. Synthesis of 387

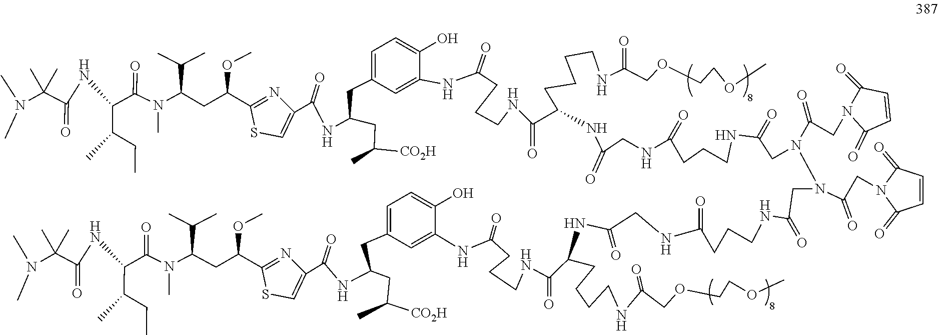

To a solution of compound 383 (121 mg, 0.050 mmol) dissolved in DMF (3 mL) and cooled over an ice water bath was added perfluorophenyl 2-((6S, 9R, 11R)-6-((S)-sec-butyl)-9-isopropyl-2, 3, 3, 8-tetramethyl-4, 7-dioxo-12-oxa-2, 5, 8-triazatridecan-11-yl)thiazole-4-carboxylate (80.0 mg, 0.120 mmol), followed by addition of DIPEA (0.15 mL). The reaction was then stirred at r.t. for 2 hours and then adjusted to pH 4-5 using formic acid. The mixture was concentrated, re-dissolved in a small amount of water, and purified by preparative C-18 HPLC (10-60% MeCN/H₂O containing 0.1% formic acid). Fractions containing the product were combined, concentrated and lyophilized to give a colorless foam (106.2 mg, 63% yield). ESI m/z calcd. for C₁₅₈H₂₅₇N₂₆O₅₀S₂ [M+H]⁺: 3382.7809, found: 3382.7940.

### Example 424. Synthesis of 388

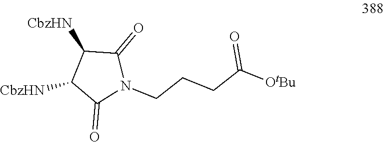 [SMILES: CC(C)(C)OC(=O)CCCN1C(=O)[C@H](NC(=O)OCc2ccccc2)[C@@H](NC(=O)OCc2ccccc2)C1=O]

To a solution of compound 127 (12.4 g, 22.2 mmol) in DMF (100 mL) at about 0° C., HATU (16.9 g, 44.5 mmol) and TEA (6.2 mL, 44.5 mmol) were added. The mixture was stirred at room temperature overnight, then concentrated, then diluted with water (200 mL), extracted with ethyl acetate (3×100 mL). The organic phase was washed with water (50 mL), 5% NaHCO₃ (50 mL), 2 N HCl (50 mL), dried over anhydrous sodium sulfate, filtered and concentrated. The residue was recrystallized with petroleum and ethyl acetate to give the title compound, as a yellow solid (10.0 g, 83.3% yield). ESI-MS m/z: calcd. for C₂₈H₃₄N₃O₈ [M+H]⁺: 540.23; found 540.23.

### Example 425. Synthesis of 389

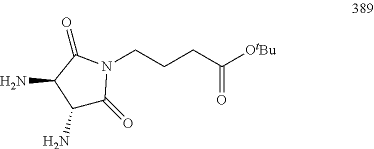 [SMILES: CC(C)(C)OC(=O)CCCN1C(=O)[C@H](N)[C@@H](N)C1=O]

A mixture of compound 388 (10.0 g, 18.5 mmol) and 10% palladium carbon (1.0 g) in methanol (100 mL) was stirred under hydrogen for 3 h. The solid was filtered off and filtrate concentrated to give a colorless oil (4.6 g, 91% yield). ESI m/z: calcd. for C₁₂H₂₂N₃O₄ [M+H]⁺: 272.15; found 272.15.

### Example 426. Synthesis of 390

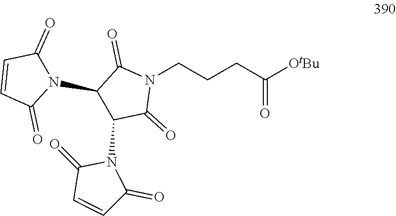 [SMILES: CC(C)(C)OC(=O)CCCN1C(=O)[C@H](N2C(=O)C=CC2=O)[C@@H](N2C(=O)C=CC2=O)C1=O]

Compound 389 (2.8 g, 10.4 mmol) was dissolved in a saturated solution of NaHCO₃ (40 mL) and cooled to about 5° C., N-(methoxycarbonyl)maleimide (3.2 g, 20.8 mmol) was added to the stirred solution. The mixture was stirred at 0° C. for 2 h and r.t. for 2 h, and then diluted with ice water (100 mL), extracted with ethyl acetate (3×50 mL). The combined organic phase was washed with water (50 mL), dried over anhydrous sodium sulfate, filtered and concentrated, purified by column chromatography (1-70% ethyl acetate/petroleum ether) to give compound 390 (750 mg, 17% yield). ESI m/z: calcd. for C₂₀H₂₂N₃O₈ [M+H]⁺: 432.13, found 432.13.

### Example 427. Synthesis of 391

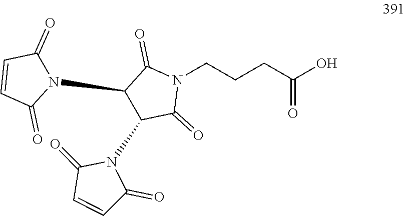 [SMILES: O=C(O)CCCN1C(=O)[C@H](N2C(=O)C=CC2=O)[C@@H](N2C(=O)C=CC2=O)C1=O]

Compound 390 (750 mg, 1.7 mmol) was dissolved in dichloromethane (5 mL), and treated with TFA (5 mL) at r.t. for 2 hours. The mixture was concentrated to give a white solid (652 mg, 100% yield). ESI-MS m/z: calcd. for C₁₆H₁₄N₃O₈ [M+H]⁺: 376.07, found 376.07.

### Example 428. Synthesis of 392

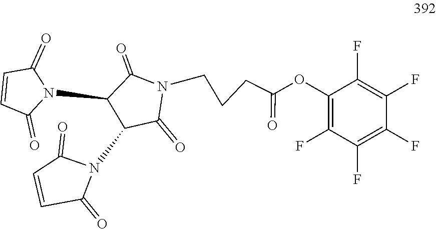 [SMILES: O=C(CCCN1C(=O)[C@H](N2C(=O)C=CC2=O)[C@@H](N2C(=O)C=CC2=O)C1=O)Oc1c(F)c(F)c(F)c(F)c1F]

To a solution of compound 391 (400 mg, 1.07 mmol) in dichloromethane (5 mL), EDC (410 mg, 2.14 mmol) and pentafluorophenol (394 mg, 2.14 mmol) were added. The reaction mixture was stirred for 1 hour and then diluted with dichloromethane (50 mL), washed with water (10 mL), dried over anhydrous sodium sulfate, filtered and concentrated to afford the title compound (578 mg, 100% yield). ESI-MS m/z calcd. for C₂₂H₁₃F₅N₃O₈ [M+H]⁺ 542.05, found 542.05.

### Example 429. Synthesis of 393

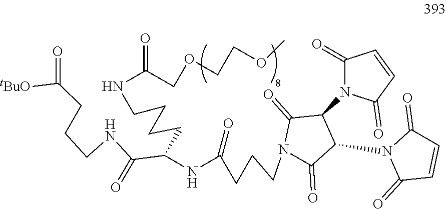

To a solution of compound 149 (0.91 g, 1.28 mmol) and compound 392 (578 mg, 1.07 mmol) in DMF (8 mL), DIPEA (373 μL, 2.14 mmol) was added at 0° C. The mixture was warmed to r.t. and stirred for 1 hour, then diluted with dichloromethane (50 mL) and washed with water (20 mL), 2N HCl (20 mL) and water (20 mL), dried over anhydrous sodium sulfate, filtered, concentrated and purified by silica gel column chromatography (100:1 to 10:1 dichloromethane/methanol) to give the title compound (0.60 g, 55% yield). ESI-MS m/z: calcd. for C₄₉H₇₇N₆O₂₀[M+H]⁺: 1069.51, found 1069.51.

### Example 430. Synthesis of 394

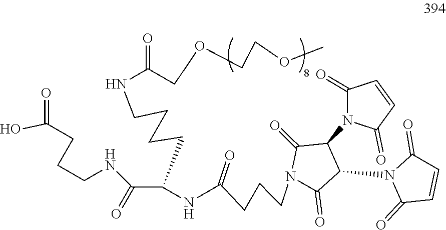

Compound 393 (0.60 g, 0.56 mmol) was dissolved in TFA (3 mL) and dichloromethane (3 mL) and stirred at room temperature for 2 hours. The reaction mixture was concentrated and co-evaporated with toluene twice, and the residue was placed on a vacuum pump to give the title compound (0.32 g, 57% yield). ESI MS m/z calcd. for C₄₅H₆₉N₆O₂₀[M+H]⁺: 1013.45, found 1013.45.

### Example 431. Synthesis of 395

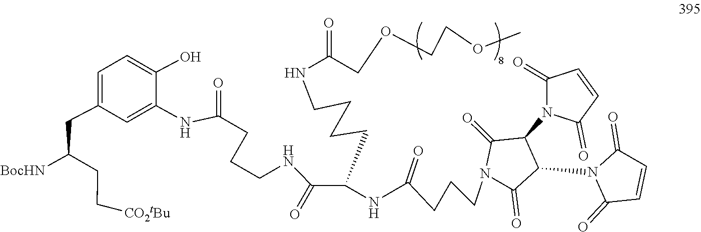

To a solution of compound 394 (0.20 g, 0.197 mmol) and tert-butyl (R)-5-(3-amino-4-hydroxyphenyl)-4-((tert-butoxycarbonyl)amino)pentanoate (90 mg, 0.237 mmol) in dichloromethane (10 mL) was added EDC HCl (76 mg, 0.394 mmol). The mixture was stirred for 1 hour, then diluted with dichloromethane (50 mL) and washed with water (20 mL), dried over anhydrous sodium sulfate, filtered, concentrated and purified on silica gel column, eluted with MeOH/DCM (1:10) to afford compound 395 (150 mg, 56% yield). ESI-MS m/z: calcd. for C₆₅H₉₉N₈O₂₄ [M+H]⁺: 1375.67, found 1375.67.

### Example 432. Synthesis of 396

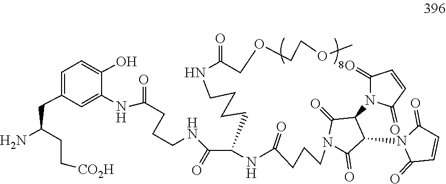

Compound 395 (0.60 g, 0.044 mmol) was dissolved in TFA (3 mL) and dichloromethane (3 mL) and stirred at room temperature for 2 hours. The reaction mixture was concentrated and co-evaporated with toluene twice, and the residue was placed on a vacuum pump to give the title compound (53 mg, 100% yield). ESI-MS m/z: calcd. for C₅₆H₈₃N₈O₂₂ [M+H]⁺: 1219.55, found 1219.55.

### Example 433. Synthesis of 397

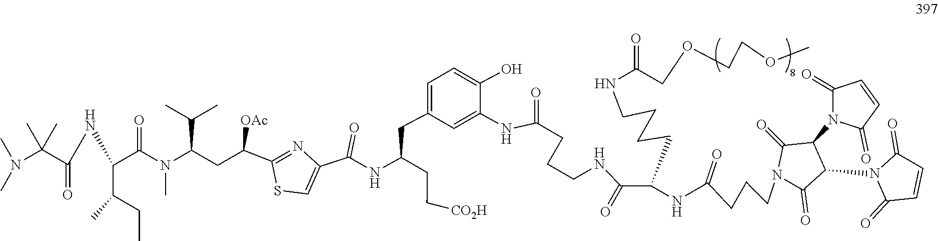

To a solution of compound 396 (53 mg, 0.044 mmol) and perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (30 mg, 0.044 mmol) dissolved in DMF (3 mL) and cooled over an ice water bath was added DIPEA (30 μL, 0.176 mmol). The reaction was then stirred at r.t. for 1 hour and purified by preparative C-18 HPLC (10-60% MeCN/H₂O containing 0.1% formic acid). Fractions containing the product were combined, concentrated and lyophilized to give a colorless foam (35 mg, 46% yield). ESI-MS m/z calcd. for C₈₁H₁₂₃N₁₂O₂₇S [M+H]⁺: 1727.83, found: 1727.83.

### Example 434. Synthesis of 398

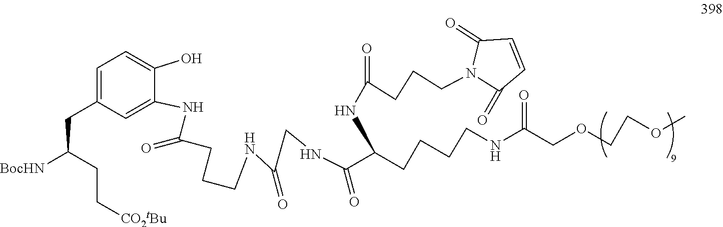

To a solution of(S)-37-(4-(2,5-dioxo-2,5-dihydro-1H-pyrrol-1-yl)butanamido)-31,38,41-trioxo-2,5,8,11,14,17,20,23,26,29-decaoxa-32,39,42-triazahexatetracontan-46-oic acid (0.30 g, 0.30 mmol) and tert-butyl (R)-5-(3-amino-4-hydroxyphenyl)-4-((tert-butoxycarbonyl)amino)-pentanoate (0.12 g, 0.40 mmol) in DCM (20 mL) was added EDC (0.96 g, 0.50 mmol). The mixture was stirred for 2 hours, washed with water and brine, dried over anhydrous sodium sulfate, filtered and concentrated to afford the title compound (0.60 g, 100% yield). ESI m/z calcd. for C₆₁H₁₀₁N₇O₂₂ [M+H]⁺: 1285.51, found: 1284.97.

### Example 435. Synthesis of 399

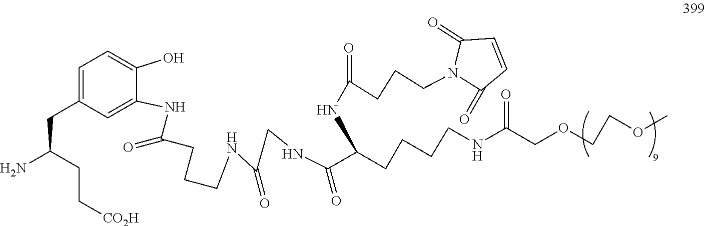

A solution of compound 398 (0.51 g, 0.40 mmol) in dichloromethane (10 mL) was treated with formic acid (5 mL) at r.t. for 1 h, then concentrated and purified by reverse phase HPLC (C₁₈ column, 10-80% acetonitrile/water in 40 min, v=8 mL/min) to afford the title compound (0.21 g, 48% yield). ESI-MS m/z calcd. for C₅₂H₈₅N₇O₂₀ [M+H]⁺: 1129.28, found: 1128.85.

### Example 436. Synthesis of 400

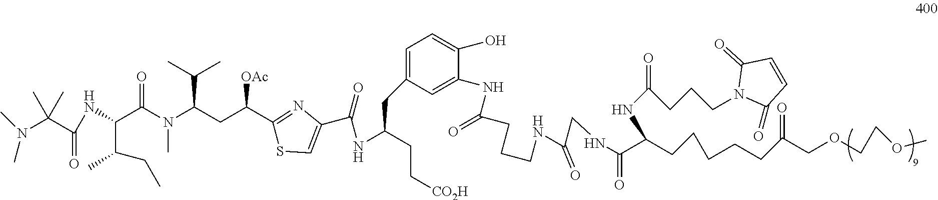

To a mixture of perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (0.13 g, 0.19 mmol) and compound 399 (0.21 g, 0.19 mmol) in DMF (5 mL) was added DIPEA (74 mg, 0.57 mmoL) at 0° C. The reaction was stirred at 0° C. for 1 h and room temperature for 1 h, concentrated under high vacuum, dissolved in small amount of water and then purified by prep-HPLC (C₁₈ column, 10-90% MeCN/H₂O) to give the title compound (54 mg, 17% yield). ESI MS m/z calcd. for C₇₇H₁₂₅N₁₁O₂₅S [M+H]⁺ 1637.96, found 1638.40.

### Example 437. Synthesis of 401a/b

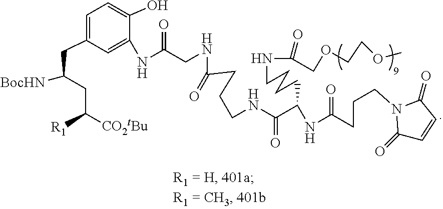

A solution of 2,5-dioxopyrrolidin-1-yl (S)-(37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-oyl)glycinate (1.00 g, 0.98 mmol) and tert-butyl (R)-5-(3-amino-4-hydroxyphenyl)-4-((tert-butoxycarbonyl)amino)pentanoate (0.315 g, 0.98 mmol) or (2S,4R)-tert-butyl 5-(3-amino-4-hydroxyphenyl)-4-((tert-butoxycarbonyl)amino)-2-methylpentanoate (0.386 g, 0.98 mmol) in THF (15 mL) was heated at 60° C. overnight and then concentrated, purified by column chromatography (MeOH/dichloromethane 1:10) to afford the compound 401a (0.75 g, 59% yield). ESI MS m/z calcd. for C₆₁H₁₀₁N₇O₂₂ [M+H]⁺: 1283.70, found: 1284.71; or 401b (0.801 g, 63% yield). ESI MS m/z calcd. for C₆₂H₁₀₃N₇O₂₂ [M+H]⁺: 1297.72, found: 1298.85.

### Example 438. Synthesis of 402a/b

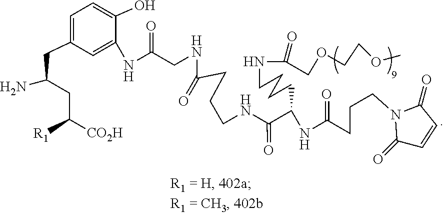

A solution of compound 401a or 401b (0.58 mmol) in dichloromethane (5 mL) was treated with TFA (3 mL) at r.t. for 0.5 h, diluted with toluene then concentrated, dried over oil pump to afford the title compound 402a or 402b as a yellow oil (-99% yield) which was used for the next step without further purification. 402a, ESI-MS m/z calcd. for C₅₂H₈₅N₇O₂₀ [M+H]⁺: 1127.58, found: 1128.60; 402 b, ESI-MS m/z calcd. for C₅₃H₈₇N₇O₂₀ [M+H]⁺: 1141.59, found: 1141.61.

### Example 439. Synthesis of 403a/b

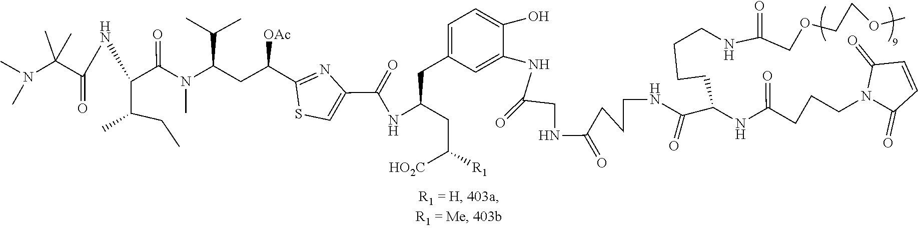

To a mixture of perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (0.32 g, 0.47 mmol) and compound 402a or 402b (0.47 mmol) in DMF (5 mL) was added DIPEA (120 mg, 0.94 mmoL) at 0° C. The reaction was stirred at room temperature for 1 h, concentrated under high vacuum, dissolved in small amount of water and then purified by prep-HPLC (C18 column, 10-90% MeCN/H₂O) to give the compound 403a or 403b (42%˜65% yield). 403a ESI MS m/z calcd. for C₇₇H₁₂₅N₁₁O₂₅S [M+H]⁺ 1635.86, found 1636.87; 403b ESI MS m/z calcd. for C₇₈H₁₂₇N₁₁O₂₅ [M+H]⁺ 1649.87, found 1650.89.

### Example 440. Synthesis of 404

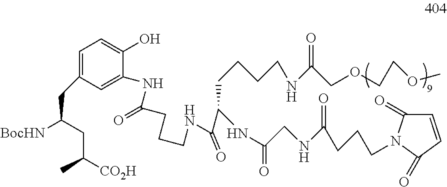

A solution of 2,5-dioxopyrrolidin-1-yl (S)-37-(2-(4-(2,5-dioxo-2,5-dihydro-1H-pyrrol-1-yl)butanamido)acetamido)-31,38-dioxo-2,5,8,11,14,17,20,23,26,29-decaoxa-32,39-diazatritetracontan-43-oate (1.56 g, 1.64 mmol) and compound 311 (0.67 g, 1.97 mmol) in THF (20 mL) was heated at 60° C. overnight and then concentrated, purified by column chromatography (MeOH/dichloromethane 1:10) to afford the title compound (1.72 g, 84% yield). ESI MS m/z calcd. for C₅₈H₉₅N₇O₂₂ [M+H]⁺: 1243.43, found: 1242.65.

### Example 441. Synthesis of 405

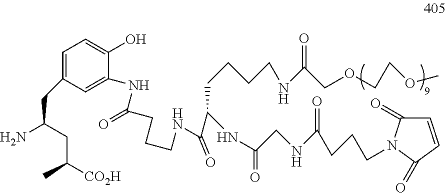

A solution of compound 404 (1.72 g, 1.38 mmol) in dichloromethane (10 mL) was treated with TFA (5 mL) at r.t. for 0.5 h, then concentrated to afford the title compound as a yellow oil (0.62 g, >100% yield). ESI-MS m/z calcd. for C₅₃H₈₇N₇O₂₀ [M+H]⁺: 1143.31, found: 1142.60.

### Example 442. Synthesis of 406

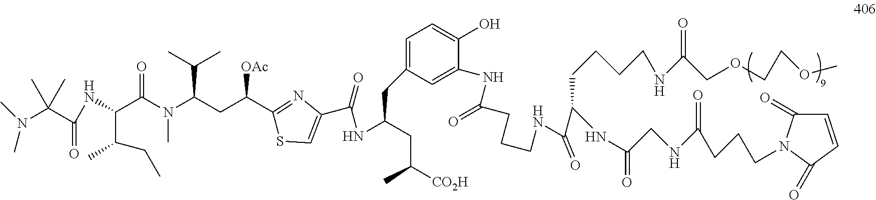

To a mixture of perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (0.45, 0.65 mol) and compound 405 from previous step (0.57 g, 0.50 mmol) in DMF (5 mL) was added DIPEA (260 mg, 2.0 mmoL) at 0° C. The reaction was stirred at room temperature for 1 h, concentrated under high vacuum, dissolved in small amount of water and then purified by prep-HPLC (C18 column, 10-90% MeCN/H₂O) to give the title compound (0.28 g, 34% yield). ESI MS m/z calcd. for C₇₈H₁₂₇N₁₁O₂₅S [M+H]⁺ 1651.99, found 1650.87.

### Example 443. Synthesis of Compound 407a/b/c/d/e/f

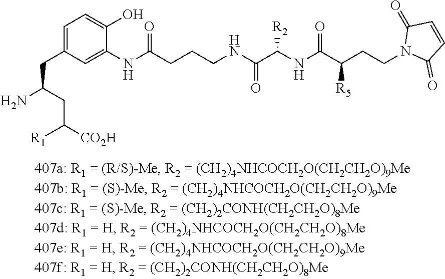

A solution of Boc-protected amine (7.0 mmol) respectively in dichloromethane (10 mL) was treated with TFA (10 mL) at r.t. for 2 h then concentrated and co-evaporated with toluene to give crude product 407a, 407b, 407c, 407d, 407e or 407f respectively, which was used directly in the next step.

### Example 444. Synthesis of Compound 408a/b/c/d/e/f

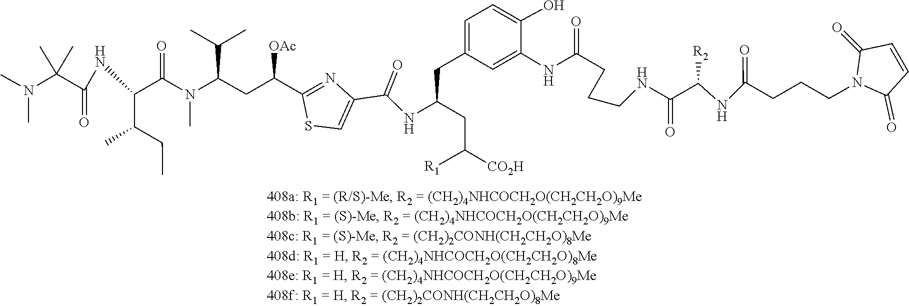

Compound 407a, 407b, 407c, 407d, 407e or 407f (7.0 mmol) respectively and perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl)thiazole-4-carboxylate (5.2 g, 7.5 mmol) were dissolved in DMA (20 mL). And then DIPEA (4.8 mL, 28 mmol) was added. The resulting mixture was stirred at r.t. for 3 h. After the solvent was removed under vacuum, the residue was purified on preparative HPLC (C₁₈ column, 10-90% MeCN/H₂O) to afford the title product 408a, 408b, 408c, 408d, 408e or 408f respectively. 408a: 9.26 g, 83% yield, MS ESI m/z calcd for C₇₆H₁₂₄N₁₀O₂₄S [M+H]⁺: 1593.85, found: 1594.06; 408b: 9.32 g, 86% yield, MS ESI m/z calcd for C₇₄H₁₂₀N₁₀O₂₃S [M+H]⁺: 1549.82, found: 1549.74; 408c: 8.76 g, 84% yield, MS ESI m/z calcd for C₇₁H₁₁₄N₁₀O₂₂S [M+H]⁺: 1491.78, found: 1491.87; 408d: 8.49 g, 79% yield, MS ESI m/z calcd for C₇₃H₁₁₈N₁₀O₂₃S [M+H]⁺: 1535.81, found: 1535.93; 408e: 8.73 g, 78% yield, MS ESI m/z calcd for C₇₅H₁₂₂N₁₀O₂₄S [M+H]⁺: 1579.84, found: 1579.92; 408f: 8.27 g, 80% yield, MS ESI m/z calcd for C₇₀H₁₁₂N₁₀O₂₂S [M+H]⁺: 1477.77 found: 1476.82.

### Example 445. Synthesis of Compound 409a/b/c

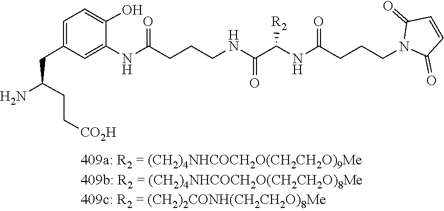

A solution of Boc-protected amine (5.0 mmol) in dichloromethane (5 mL) was treated with TFA (5 mL) at r.t. for 2 h then concentrated and co-evaporated with toluene to give crude product 409a, 409b or 409c, which was used directly in the next step.

### Example 446. Synthesis of Compound 410a/b/c

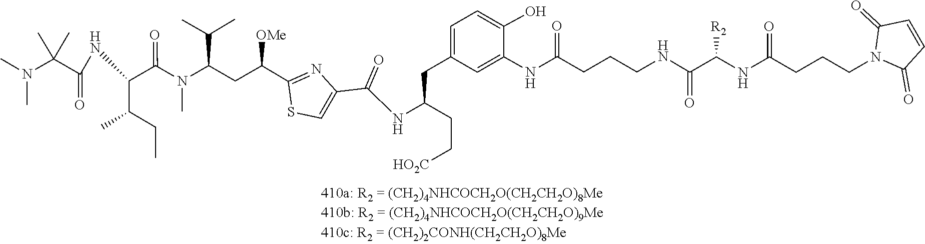

Compound 409a, 409b or 409c (1.0 mmol) respectively and perfluorophenyl 2-((6S,9R,11R)-6-((S)-sec-butyl)-9-isopropyl-2,3,3,8-tetramethyl-4,7-dioxo-12-oxa-2,5,8-triazatridecan-11-yl)thiazole-4-carboxylate (4.98 g, 1.2 mmol) were dissolved in DMF (10 mL). And then DIPEA (0.86 mL, 5.0 mmol) was added. The resulting mixture was stirred at r.t. for 3 h. After the solvent was removed under vacuum, the residue was purified on preparative HPLC (C₁₈ column, 10-90% MeCN/H₂O) to afford the title product 410a, 410b or 410c respectively. 410a: 1.25 g, 82% yield, MS ESI m/z calcd for C₇₃H₁₂₀N₁₀O₂₂S [M+H]⁺: 1521.83, found: 1522.54; 410b: 1.38 g, 88% yield, MS ESI m/z calcd for C₇₅H₁₂₄N₁₀O₂₃S [M+H]⁺: 1565.86, found: 1566.58; 410c: 1.20 g, 83% yield, MS ESI m/z calcd for C₆₉H₁₁₂N₁₀O₂₁S [M+H]⁺: 1449.77 found: 1449.45.

### Example 447. Synthesis of Compound 411a/b/c/d/e/f

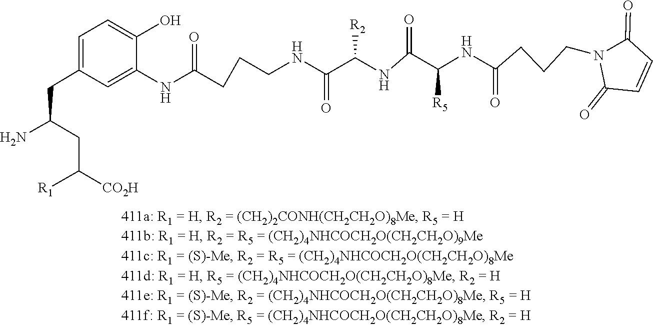

A solution of Boc-protected amine (7.0 mmol) in dichloromethane (20 mL) was treated with TFA (5 mL) at r.t. for 0.5 h then concentrated and co-evaporated with toluene to give crude product 411a, 411b, 411c, 411d, 4Ile or 411f respectively, which was used directly in the next step.

### Example 448. Synthesis of Compound 412a/412b/412c/412d/412e/412f

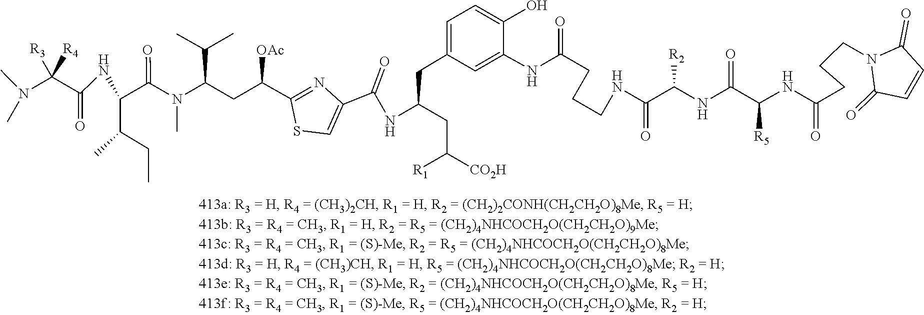

Compound 411a, 411b, 411c, 411d, 4Ile or 411f (1.0 mmol) respectively and compound perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate or perfluorophenyl 2-((3S, 6S, 9R, 11R)-6-((S)-sec-butyl)-3, 9-diiso-propyl-2, 8-dimethyl-4, 7, 13-trioxo-12-oxa-2, 5, 8-triazatetradecan-11-yl)thiazole-4-carboxylate (1.2 mmol) were dissolved in DMF (5 mL). And then DIPEA (0.86 mL, 5 mmol) was added. The resulting mixture was stirred at r.t. for 3 h. After the solvent was removed under vacuum, the residue was purified on preparative HPLC (C₁₈ column, 10-90% MeCN/H₂O) to afford product 412a, 412b, 412c, 412d, 412e or 412f respectively. 412a: 1.27 g, 82% yield, MS ESI m/z calcd for C₇₃H₁₁₇N₁₁O₂₃S [M+H]⁺: 1548.80 found: 1549.35; 412b: 1.68 g, 78% yield, MS ESI m/z calcd for C₁₀₂H₁₇₄N₁₂O₃₆S [M+H]⁺: 2176.19 found: 2177.95; 412c: 1.61 g, 77% yield, MS ESI m/z calcd for C₉₉H₁₆₉N₁₂O₃₄S [M+H]⁺: 2102.16 found: 2104.15; 412d: 1.17 g, 73% yield, MS ESI m/z calcd for C₇₆H₁₂₄N₁₁O₂₄S [M+H]⁺: 1606.85 found: 1607.95; 412e: 1.20 g, 75% yield, MS ESI m/z calcd for C₇₆H₁₂₄N₁₁O₂₄S [M+H]⁺: 1606.85 found: 1607.70; 412f: 1.19 g, 74% yield, MS ESI m/z calcd for C₇₆H₁₂₄N₁₁O₂₄S [M+H]⁺: 1606.85 found: 1607.90.

### Example 449. Synthesis of Compound 413a/b/c/d

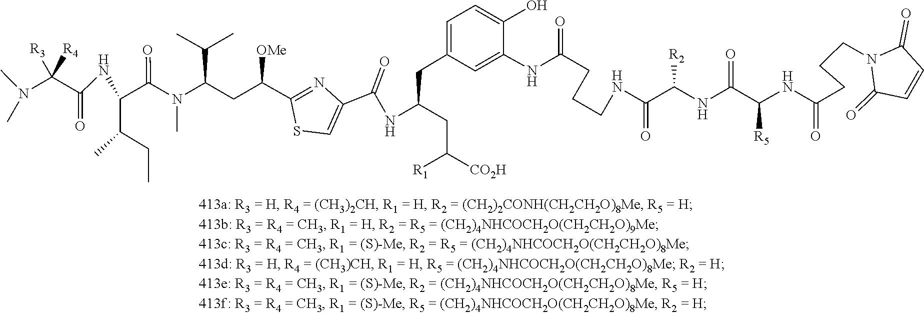

Compound 411a, 411b, 411c, 411d, 4Ile or 411f (1.0 mmol) respectively and compound perfluorophenyl 2-((6S,9R,11R)-6-((S)-sec-butyl)-9-isopropyl-2,3,3,8-tetramethyl-4,7-dioxo-12-oxa-2,5,8-triazatridecan-11-yl)thiazole-4-carboxylate or perfluorophenyl 2-((3S, 6S, 9R, 11R)-6-((S)-sec-butyl)-3, 9-diisopropyl-2, 8-dimethyl-4, 7-dioxo-12-oxa-2, 5, 8-triazatridecan-11-yl)thiazole-4-carboxylate (1.15 mmol) were dissolved in DMF (20 mL). And then DIPEA (4.8 mL, 28 mmol) was added. The resulting mixture was stirred at r.t. for 3 h. After the solvent was removed under vacuum, the residue was purified on preparative HPLC (C18 column, 10-90% MeCN/H₂O) to afford product 413a, 413b, 413c, 413d, 413e or 413f respectively. 413a: 1.20 g, 78% yield, MS ESI m/z calcd for C₇₂H₁₁₇N₁₁O₂₂S [M+H]⁺: 1520.80 found: 1521.65; 412b: 1.33 g, 62% yield, MS ESI m/z calcd for C₁₀₁H₁₇₄N₁₂O₃₅S [M+H]⁺: 2148.19 found: 2149.95; 412c: 1.55 g, 74% yield, MS ESI m/z calcd for C₉₈H₁₆₉N₁₂O₃₃S [M+H]⁺: 2074.16 found: 2075.15; 412d: 1.14 g, 72% yield, MS ESI m/z calcd for C₇₅H₁₂₄N₁₁O₂₃S [M+H]⁺: 1578.85 found: 1579.90; 412e: 1.18 g, 75% yield, MS ESI m/z calcd for C₇₅H₁₂₄N₁₁O₂₃S [M+H]⁺: 1578.85 found: 1579.55; 412f: 1.16 g, 73% yield, MS ESI m/z calcd for C₇₅H₁₂₄N₁₁O₂₃S [M+H]⁺: 1578.85 found: 1579.75.

### Example 450. Synthesis of Compound 414a/b/c/d

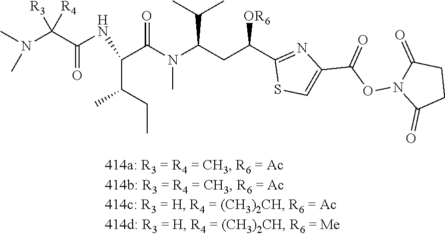

To a solution of acid (10.0 mmol) in dichloromethane (30 mL) were added N-hydroxysuccinimide (1.38 g, 12.0 mmol) and EDC HCl (2.30 g, 12.0 mmol). The reaction mixture was stirred at r.t. for 3 h and then concentrated. The residue was purified on silica gel column (50%-80% PE/EtOAc) to give the title compound 414a, 414b, 414c or 414d respectively. 414a: 5.47 g, 88% yield, ESI MS m/z calcd for C₂₉H₄₅N₅O₈S [M+H]⁺ 624.28, found 624.58; 414b: 4.87 g, 82% yield, ESI MS m/z calcd for C₂₈H₄₅N₅O₇S [M+H]⁺ 596.29, found 596.25; 414c: 5.41 g, 85% yield, ESI MS m/z calcd for C₃₀H₄₇N₅O₈S [M+H]⁺ 638.30, found 638.85; 414d: 5.17 g, 85% yield, ESI MS m/z calcd for C₂₉H₄₇N₅O₇S [M+H]⁺ 610.30, found 610.56.

### Example 451. Synthesis of Compound 415a/b/c/d

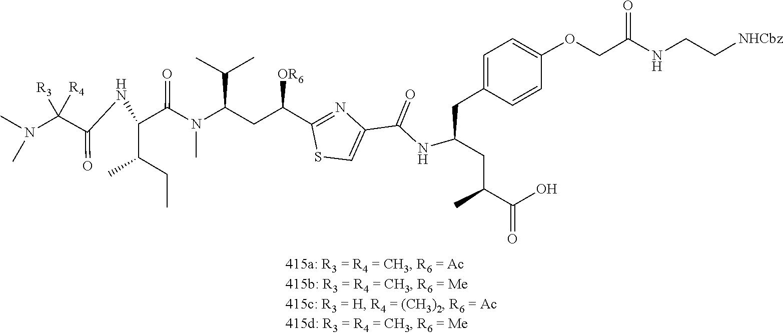

A mixture of (2S,4R)-4-amino-5-(4-(2-((2-(((benzyloxy)carbonyl)amino)ethyl) amino)-2-oxoethoxy)phenyl)-2-methylpentanoic acid (457 mg, 1.0 mmol) and compound 414a, 414b, 414c or 414d (1.0 mmol) respectively in 0.1 M NaH₂PO₄ (10 mL) and EtOH (10 mL) was stirred at r.t. overnight, and then concentrated, purified by SiO₂ column chromatography (5%-20% MeOH/DCM) to yield the title compound 415a, 415b, 415c or 415d respectively. 415a: 733 mg, 76% yield, ESI MS m/z calcd for C₄₉H₇₂N₇O₁₁S [M+H]⁺ 966.50, found 966.50. 415b: 685 mg, 73% yield, ESI MS m/z calcd for C₄₈H₇₂N₇O₁₀S [M+H]⁺ 938.50, found 938.50; 415c: 713 mg, 72% yield, ESI MS m/z calcd for C₅₀H₇₄N₇O₁₁S [M+H]⁺ 980.51, found 980.55; 415d: 667 mg, 70% yield, ESI MS m/z calcd for C₄₉H₇₄N₇O₁₀S [M+H]⁺ 952.52, found 952.55.

### Example 452. Synthesis of Compound 416a/416b/416c/416d

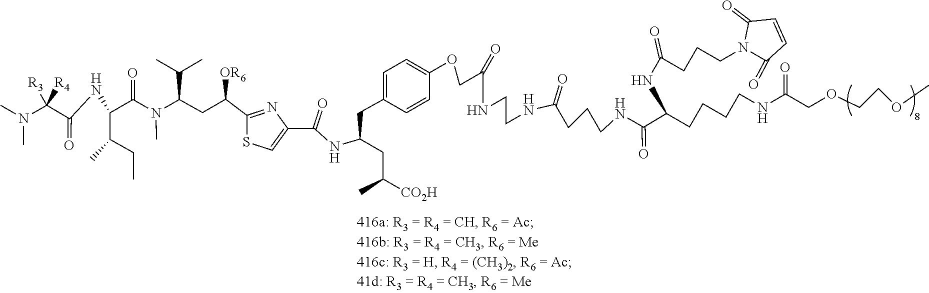

Compound 415a, 415b, 415c or 415d (0.20 mmol) respectively was dissolved in methanol (20 mL) and hydrogenated (1 atm H₂) with Pd/C catalyst (10 wt %, 20 mg) at r.t. for 4 h. The catalyst was filtered off and the filtrate were concentrated under reduced pressure to afford an amino intermediate compound (97%-102% yield) as a brown foamy solid which was used directly for the next step without further purification.

To a mixture of the prepared amino compound and pentafluorophenyl ester (0.23 mmol) in DMF (8 mL) was added Et₃N (0.17 mL, 1.2 mmol). The mixture was stirred at r.t. for 6 h, concentrated under high vacuum, dissolved in small amount of water and then purified by prep-HPLC (C-18 column, 10-90% MeCN/H₂O). Fractions containing the product were combined, concentrated and lyophilized to give the title compound 416a, 416b, 416c or 416d respectively. 416a: 197 mg, 61% yield (two steps), ESI MS m/z calcd for C₇₇H₁₂₆N₁₁O₂₄S [M+H]⁺ 1620.8699, found 1620.8810; 416b: 189 mg, 59% yield (two steps), ESI MS m/z calcd for C₇₆H₁₂₆N₁₁O₂₃S [M+H]⁺ 1592.8750, found 1592.8845; 416c: 209.1 mg, 64% yield, ESI MS m/z calcd for C₇₈H₁₂₈N₁₁O₂₄S [M+H]⁺ 1634.8855, found 1634.8980; 416d: 196 mg, 61% yield (two steps), ESI MS m/z calcd for C₇₇H₁₂₈N₁₁O₂₃S [M+H]⁺ 1606.8906, found 1606.9035.

### Example 453. Synthesis of Compound 417a/b

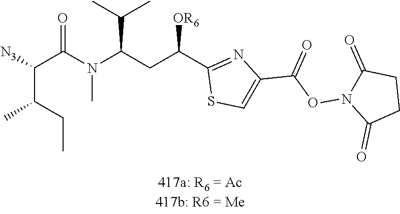

To a solution of acid (10.0 mmol) in dichloromethane (40 mL) were added N-hydroxysuccinimide (1.38 g, 12.0 mmol) and EDC HCl (2.30 g, 12.0 mmol). The reaction mixture was stirred at r.t. for 3 h and then concentrated. The residue was purified on silica gel column (50%-80% EtOAc/PE) to give the title compound 417a or 417b. 417a: 4.60 g, 86% yield, ESI MS m/z calcd for C₂₃H₃₂N₆O₇S [M+H]⁺ 537.19, found 537.88; 417b: 4.56 g, 90% yield, ESI MS m/z calcd for C₂₂H₃₂N₆O₆S [M+H]⁺ 509.19, found 509.56.

### Example 454. Synthesis of Compound 418a/b

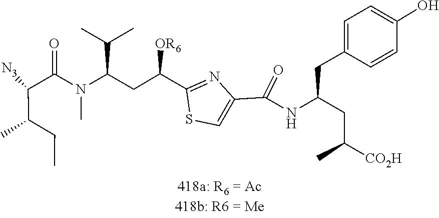

A mixture of (2S,4R)-4-amino-5-(4-hydroxyphenyl)-2-methylpentanoic acid (1.78 g, 8.0 mmol) and compound 417a or 417b (8.0 mmol) in 0.1 M NaH₂PO₄ (10 mL) and EtOH (10 mL) was stirred at r.t. overnight, and then concentrated, purified by column chromatography (50% EtOAc/PE, 0-5% MeOH/DCM) to yield the title compound 418a or 418b. 418a: 4.13 g, 80% yield, ESI MS m/z calcd for C₃₁H₄₄N₆O₇S [M+H]⁺ 645.30, found 645.96; 418b: 4.34 g, 88% yield, ESI MS m/z calcd for C₃₀H₄₄N₆O₆S [M+H]⁺ 617.30, found 617.52.

### Example 455. Synthesis of Compound 419a/b

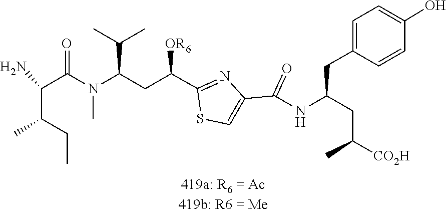

Compound 418a or 418b (6.0 mol) was dissolved in methanol (10 mL), Pd/C (10 mg, 5 wt %) was added, and the mixture was stirred under a hydrogen balloon (1 atm H₂) overnight and then filtered. The filtrate was concentrated to give the title product (assuming 100% yield), no further purification was required for the use in the next step.

### Example 456. Synthesis of Compound 420a/b

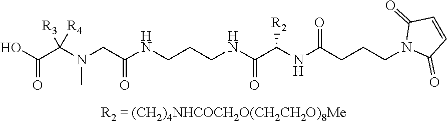

A mixture of compound (S)—N-(6-((3-aminopropyl)amino)-5-(4-(2,5-dioxo-2,5-dihydro-1H-pyrrol-1-yl)butanamido)-6-oxohexyl)-2,5,8,11,14,17,20,23,26-nonaoxaoctacosan-28-amide (15.8 g, 20 mmol), 3,3,4-trimethylmorpholine-2,6-dione or 3-isopropyl-4-methylmorpholine-2,6-dione (25 mmol) in THF (100 mL) was refluxed for 2.0 h, cooled to r.t. and concentrated. The residue was purified by prep-HPLC (C18 column, 10-90% MeCN/H₂O) to give the title compound 420a or 420b. 420a: 9.48 g, 50% yield, ESI MS m/z calcd for C₄₃H₇₆N₆O₁₇ [M+H]⁺ 949.53, found 949.89; 420b: 11.8 g, 62% yield, ESI MS m/z calcd for C₄₄H₇₈N₆O₁₇ [M+H]⁺ 963.54, found 963.52.

### Example 457. Synthesis of Compound 421a/b

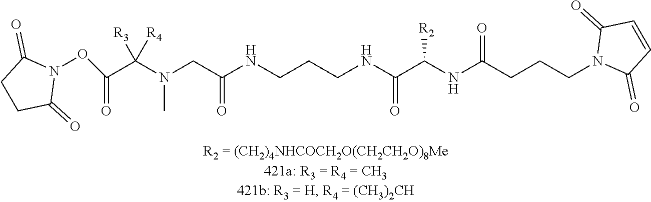

To a solution of compound 420a or 420b (10.0 mmol) in dichloromethane (40 mL) were added N-hydroxysuccinimide (1.38 g, 12.0 mmol) and EDC HCl (2.30 g, 12.0 mmol). The reaction mixture was stirred at r.t. for 3 h and then concentrated. The residue was purified on silica gel column (50%-80% EtOAc/PE) to give the title compound 421a or 421b. 421a: 9.31 g, 89% yield, ESI MS m/z calcd for C₄₇H₇₉N₇O₁₉ [M+H]⁺ 1046.54, found 1046.98; 421b: 9.33 g, 88% yield, ESI MS m/z calcd for C₄₈H₈₁N₇O₁₉ [M+H]⁺ 1060.56, found 1060.48.

### Example 458. Synthesis of Compound 422a/b/c/d

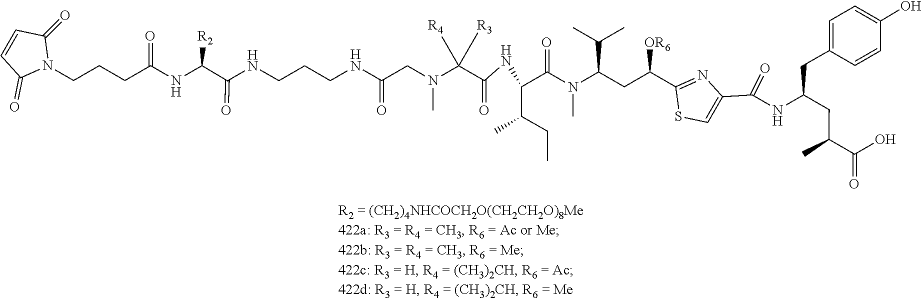

A mixture of compound 421a or 421b (1.00 mmol) and compound 419a (0.50 g, 0.80 mmol) or 419b (0.47 g, 0.80 mmol) in 0.1 M NaH₂PO₄ (1.0 mL) and EtOH (1.0 mL) was stirred at r.t. overnight, and then concentrated, dissolved in water and purified by prep-HPLC (C18 column, 10-90% MeCN/H₂O) to give the title compound 422a, 422b, 422c or 422d. 422a (R₆═Ac): 1.05 g, 85% yield, ESI MS m/z calcd for C₇₄H₁₂₀N₁₀O₂₃S [M+H]⁺ 1549.82, found 1551.33; 422b (R₆=Me): 1.02 g, 82% yield, ESI MS m/z calcd for C₇₃H₁₂₀N₁₀O₂₂S [M+H]⁺ 1521.83, found 1522.33; 422c (R₆═Ac): 0.94 g, 75% yield, ESI MS m/z calcd for C₇₅H₁₂₂N₁₀O₂₃S [M+H]⁺ 1562.84, found 1562.88; 422d (R₆=Me): 0.76 g, 62% yield, ESI MS m/z calcd for C₇₄H₁₂₂N₁₀O₂₂S [M+H]⁺ 1534.85, found 1536.88.

### Example 459. Synthesis of tert-butyl ((34S,42S,44R)-34-(4-(2,5-dioxo-2,5-dihydro-1H-pyrrol-1-yl)butanamido)-45-(4-hydroxyphenyl)-42-methyl-28,35,41-trioxo-2,5,8,11,14,17,20,23,26-nonaoxa-29,36,40-triazapentatetracontan-44-yl)carbamate (423)

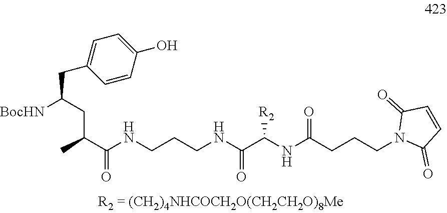

To a solution of (S)—N-(6-((3-aminopropyl)amino)-5-(4-(2,5-dioxo-2,5-dihydro-1H-pyrrol-1-yl)butanamido)-6-oxohexyl)-2,5,8,11,14,17,20,23,26-nonaoxaoctacosan-28-amide (7.91 g/, 10.0 mmol) in THF/DCM (40/40 mL) were added (2S,4R)-4-((tert-butoxycarbonyl) amino)-5-(4-hydroxyphenyl)-2-methylpentanoic acid (3.88 g, 12.0 mmol) and EDC HCl (2.30 g, 12.0 mmol). The reaction mixture was stirred at r.t. for 3 h and then concentrated. The residue was purified on silica gel column (50%-80% EtOAc/PE) to give the title compound (6.90 g, 63% yield). ESI MS m/z calcd for C₅₃H₈₈N₆O₁₈ [M+H]⁺ 1097.62, found 1098.52.

### Example 460. Synthesis of N—((S)-6-((3-((2S,4R)-4-amino-5-(4-hydroxyphenyl)-2-methylpentan-amido)propyl)amino)-5-(4-(2,5-dioxo-2,5-dihydro-1H-pyrrol-1-yl)butanamido)-6-oxohexyl)-2,5,8,11,14,17,20,23,26-nonaoxaoctacosan-28-amide (424)

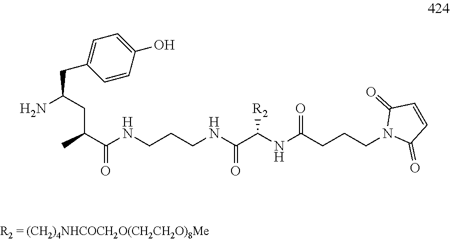

A solution of compound 423 (0.88 g, 0.8 mmol) in dichloromethane (2.5 mL) was treated with TFA (2.5 mL) at r.t. for 2 h then concentrated and co-evaporated with toluene to give crude product 424 (assuming 100% yield), which was used directly in the next step.

### Example 461. Synthesis of Compound 425a/b/c/d

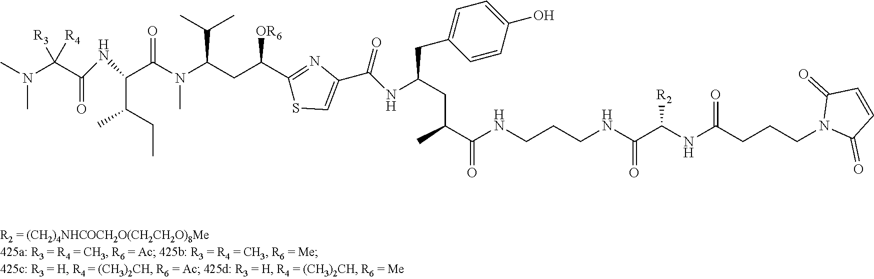

A mixture of compound 414a, 414b, 414c or 414d (1.0 mmol) and compound 424 (0.80 g, 0.8 mmol) in 0.1 M NaH₂PO₄ (2.5 mL) and EtOH (5 mL) was stirred at room temperature overnight, and then concentrated, dissolved in water and purified by prep-HPLC (C18 column, 10-90% MeCN/H₂O) to give the title compound 425a, 425b, 425c or 425d. 425a: 1.02 g, 85% yield, ESI MS m/z calcd for C₇₃H₁₂₀N₁₀O₂₁S [M+H]⁺ 1505.84, found 1506.62; 425b: 0.93 g, 79% yield, ESI MS m/z calcd for C₇₂H₁₂₀N₁₀O₂₀S [M+H]⁺ 1477.84, found 1477.60; 425c: 0.85 g, 70% yield, ESI MS m/z calcd for C₇₄H₁₂₂N₁₀O₂₁S [M+H]⁺ 1519.85, found 1520.20; 425d: 0.85 g, 71% yield, ESI MS m/z calcd for C₇₃H₁₂₂N₁₀O₂₀S [M+H]⁺ 1491.86, found 1491.80.

### Example 462. Synthesis of (4R,4′R)-di-tert-butyl 5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(28-oxo-2,5,8,11,14,17,20,23,26-nonaoxa-29-azatritriacontan-33-yl)-3,6,9,12,17,22,27,30,33,36-decaazaoctatriacontane-1,38-dioyl)bis(azanediyl))bis(4-hydroxy-3,1-phenylene))bis(4-((tert-butoxycarbonyl)amino)pentanoate) (426)

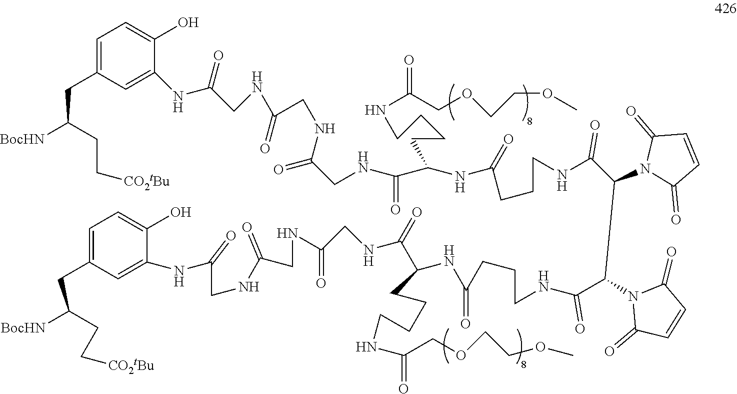

To a solution of compound 207 (1.001 g, 0.520 mmol) and (R)-tert-butyl 5-(3-amino-4-hydroxyphenyl)-4-((tert-butoxycarbonyl)amino)pentanoate (0.401 g, 1.054 mmol) in DMA (40 mL) were added EDC (0.701 g, 3.651 mmol) and DIPEA (0.20 mL, 1.15 mmol). The mixture was stirred for 8 h, concentrated under reduced pressure and purified by silica gel column chromatography with a gradient of 5-15% methanol in DCM to give the title product (1.033 g, 75% yield). MS ESI m/z calcd. for C₁₂₂H₁₉₇N₁₈O₄₆ [M+H]⁺ 2650.3630, found 2650.3820.

### Example 463. Synthesis of (4R,4′R)-5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(28-oxo-2,5,8,11,14,17,20,23,26-nonaoxa-29-azatritriacontan-33-yl)-3,6,9,12,17,22,27,30,33,36-decaazaoctatriacontane-1,38-dioyl)bis(azanediyl))bis(4-hydroxy-3,1-phenylene))bis(4-aminopentanoic acid) (427)

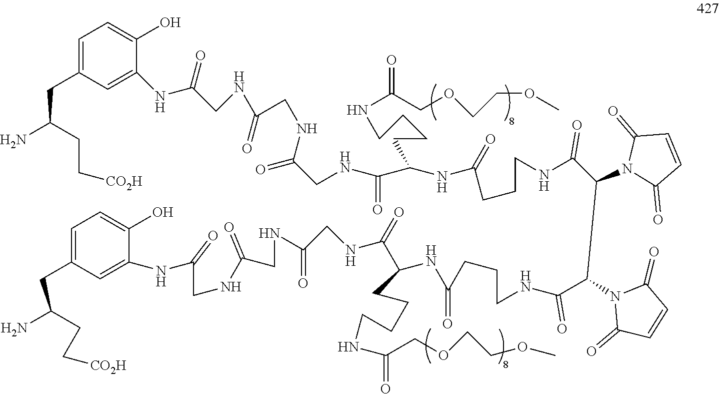

A solution of compound 426 (1.00 g, 0.377 mmol) in dioxane (10 mL) was treated with HCl (conc. 3 mL) at r.t. for 0.5 h, diluted with Toluene and dioxane (10/10 ml) and concentrated to afford the title compound as a yellow oil (0.891 g, >100% yield). ESI-MS m/z calcd. for C₁₀₄H₁₆₅N₁₈O₄₂ [M+H]⁺: 2338.1330, found: 2318.1560; C₁₀₄H₁₆₆N₁₈O₄₂ [M+2H]²+: 1169.5704, found: 1169.5785.

### Example 464. Synthesis of 428a, 428b, 428c and 428d

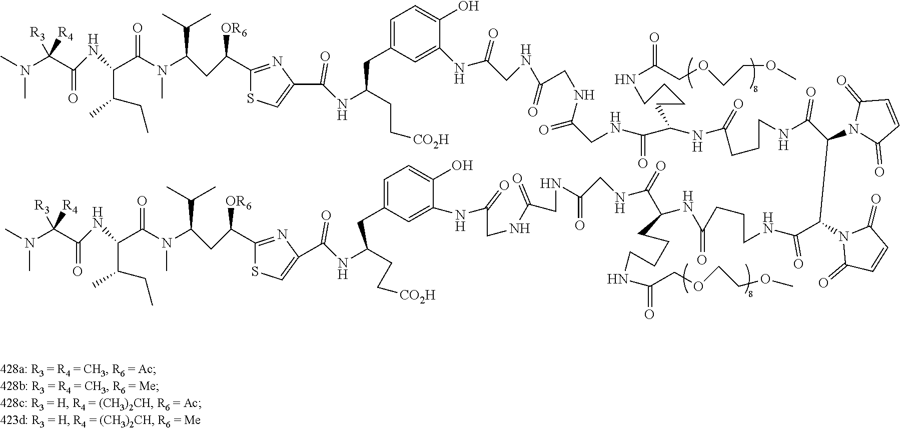

Compound 427 (200 mg, 0.0856 mmol) and perfluorophenyl 2-((6S,9R,11R)-6-((S)-sec-butyl)-9-isopropyl-2,3, 3,8-tetramethyl-4,7,13-trioxo-12-oxa-2,5,8-triazatetradecan-11-yl)thiazole-4-carboxylate (120 mg, 0.173 mmol), perfluorophenyl 2-((6S,9R,11R)-6-((S)-sec-butyl)-9-isopropyl-2,3,3,8-tetramethyl-4,7-dioxo-12-oxa-2,5,8-triazatridecan-11-yl)thiazole-4-carboxylate (120 mg, 0.180 mmol), perfluorophenyl 2-((3S,6S,9R,11R)-6-((S)-sec-butyl)-3,9-diisopropyl-2,8-dimethyl-4,7,13-trioxo-12-oxa-2,5,8-triazatetradecan-11-yl)thiazole-4-carboxylate (126 mg, 0.178 mmol), perfluorophenyl 2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxylate (122 mg, 0.180 mmol) respectively were dissolved in DMA (10 mL). And then DIPEA (0.1 mL, 0.575 mmol) was added to each of the reaction. The resulting mixture was stirred at r.t. for 3 h. After the solvent was removed under vacuum, the residue was purified on preparative HPLC (C₁₈ column, 10-60% MeCN/H₂O in 50 min, d 20×250 mm, v=10 ml/min) to afford the title product 428a, 428b, 428c, and 428d respectively. 428a: 192.3 mg, 67% yield, MS ESI m/z calcd for C₁₅₄H₂₄₅N₂₆O₅₂S₂[M+H]⁺: 3354.6768, found: 3354.6915; 428b: 193.6 mg, 69% yield, MS ESI m/z calcd for C₁₅₂H₂₄₅N₂₆O₅OS₂ [M+H]⁺: 3298.6870, found: 3298.7025; 428c: 188.6 mg, 65% yield, MS ESI m/z calcd for C₁₅₆H₂₄₉N₂₆O₅₂S₂[M+H]⁺: 3382.7081, found: 3382.7140; 428d: 199.3 mg, 69% yield, MS ESI m/z calcd for C₁₅₄H₂₄₉N₂₆O₅OS₂ [M+H]⁺: 3326.7183, found: 3326.7980.

### Example 465. Synthesis of (2S,2'S,4R,4′R)-di-tert-butyl 5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(28-oxo-2,5,8,11,14,17,20,23,26-nonaoxa-29-azatritriacontan-33-yl)-3,6,9,12,17,22,27,30,33,36-decaazaoctatriacontane-1,38-dioyl)bis(azanediyl))bis(4-hydroxy-3,1-phenylene))bis(4-((tert-butoxycarbonyl)amino)-2-methylpentanoate) (429)

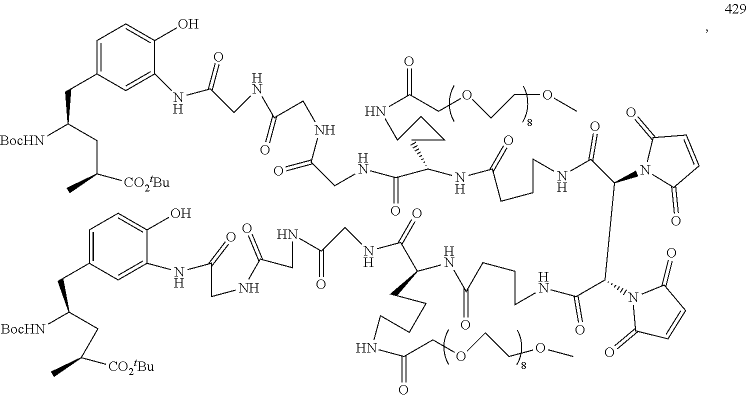

To a solution of compound 207 (1.051 g, 0.546 mmol) and (2S,4R)-tert-butyl 5-(3-amino-4-hydroxyphenyl)-4-((tert-butoxycarbonyl)amino)-2-methylpentanoate (0.451 g, 1.143 mmol) in DMA (40 mL) were added EDC (0.851 g, 4.432 mmol) and DIPEA (0.30 mL, 1.725 mmol). The mixture was stirred for 8 h, concentrated under reduced pressure and purified by silica gel column chromatography with a gradient of 5-15% methanol in DCM to give the title product (1.155 g, 79% yield). MS ESI m/z calcd. for C₁₂₄H₂₀₁N₁₈O₄₆ [M+H]⁺ 2678.3943, found 2678.4025.

### Example 466. Synthesis of (25,2'S,4R,4′R)-5,5′-((((11S,19S,20S,28S)-19,20-bis(2,5-dioxo-2,5-dihydro-1H-pyrrol-1-yl)-4,7,10,13,18,21,26,29,32,35-decaoxo-11,28-bis(28-oxo-2,5,8,11,14,17,20,23,26-nonaoxa-29-azatritriacontan-33-yl)-3,6,9,12,17,22,27,30,33,36-decaazaoctatriacontane-1,38-dioyl)bis(azanediyl))bis(4-hydroxy-3,1-phenylene))bis(4-amino-2-methylpentanoic acid) (430)

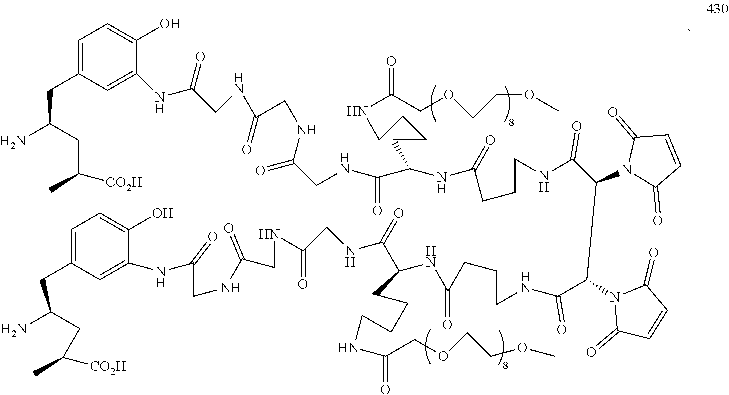

A solution of compound 429 (1.03 g, 0.384 mmol) in dioxane (10 mL) was treated with HCl (conc. 3 mL) at r.t. for 0.5 h, diluted with Toluene and dioxane (10/10 ml) and concentrated to afford the title compound as a yellow oil (0.911 g, >100% yield). ESI-MS m/z calcd. for C₁₀₆H₁₆₉N₁₈O₄₂ [M+H]⁺: 2366.1642, found: 2366.1795; C₁₀₆H₁₇₀N₁₈O₄₂ [M+2H]²+: 1183.5861, found: 1183.5970.

### Example 467. Synthesis of 431a, 431b, 431c and 431d

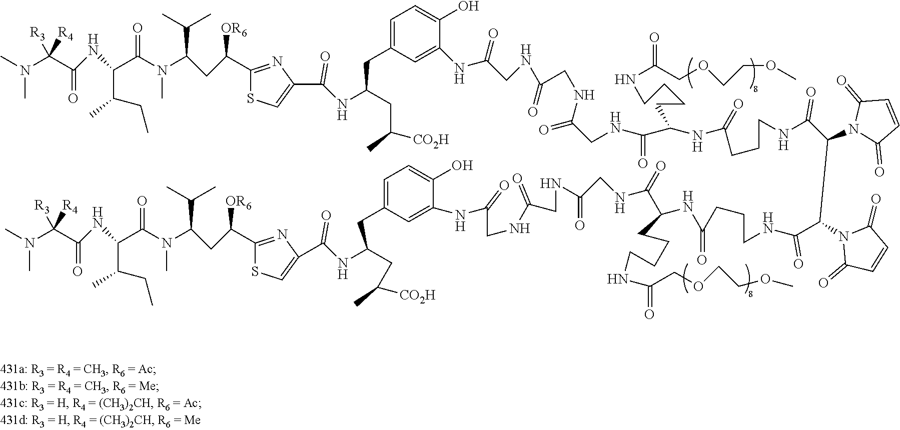

Compound 429 (210 mg, 0.0887 mmol) and perfluorophenyl 2-((6S,9R,11R)-6-((S)-sec-butyl)-9-isopropyl-2,3, 3,8-tetramethyl-4,7,13-trioxo-12-oxa-2,5,8-triazatetradecan-11-yl)thiazole-4-carboxylate (130 mg, 0.187 mmol), perfluorophenyl 2-((6S,9R,11R)-6-((S)-sec-butyl)-9-isopropyl-2,3,3,8-tetramethyl-4,7-dioxo-12-oxa-2,5,8-triazatridecan-11-yl)thiazole-4-carboxylate (128 mg, 0.192 mmol), perfluorophenyl 2-((3S,6S,9R,11R)-6-((S)-sec-butyl)-3,9-diisopropyl-2,8-dimethyl-4,7,13-trioxo-12-oxa-2,5,8-triazatetradecan-11-yl)thiazole-4-carboxylate (135 mg, 0.191 mmol), perfluorophenyl 2-((3S,6S,9R,11R)-6-((S)-sec-butyl)-3,9-diisopropyl-2,8-dimethyl-4,7-dioxo-12-oxa-2,5,8-triazatridecan-11-yl)thiazole-4-carboxylate (131 mg, 0.193 mmol) respectively were dissolved in DMA (10 mL). And then DIPEA (0.1 mL, 0.575 mmol) was added to each of the reaction. The resulting mixture was stirred at r.t. for 3 h. After the solvent was removed under vacuum, the residue was purified on preparative HPLC (C18 column, 10-60% MeCN/H₂O in 50 min, d 20×250 mm, v=10 ml/min) to afford the title product 431a, 431b, 431c, and 431d respectively. 431a: 210.2 mg, 70% yield, MS ESI m/z calcd for C₁₅₆H₂₄₉N₂₆O₅₂S₂[M+H]⁺: 3382.7081, found: 3382.7210; 431b: 207.7 mg, 69% yield, MS ESI m/z calcd for C₁₅₄H₂₄₉N₂₆O₅₀S₂ [M+H]⁺: 3326.7183, found: 3326.7320; 431c: 206.3 mg, 68% yield, MS ESI m/z calcd for C₁₅₈H₂₅₃N₂₆O₅₂S₂[M+H]⁺: 3410.7394, found: 3410.7515; 431d: 211.5 mg, 71% yield, MS ESI m/z calcd for C₁₅₆H₂₅₃N₂₆O₅₀S₂ [M+H]⁺: 3354.7496, found: 3354.7665.

### Example 468. Conjugation Reaction

Zinc amino complex (in 10-60 mM, 1.0-5.0 eq. of an antibody used) and TCEP (in 100 mM, 2.5-4.5 eq. of an antibody used) were added in sequence to a solution containing an antibody (such as BCMA, Her2, EGFR, CD33, Trop2, Steap1, CD56, PSMA and Her3 generated in house, 10-30 mg/mL, in 20 mM PBS pH 5.5-7.5) at 2-8° C. After incubation at 2-8° C. for 12-16 h (overnight), a payload/linker complex (100-200 mM, 2.0-8 0.0 eq. of the antibody used) was introduced and incubated for further 2-4 h at 2-8° C. After the incubation, cystine (100-200 mM, 4.0-8.0 eq. of the antibody) was added to the to deplete the excess TCEP, cysteine (100-200 mM, 2.0-6.0 eq. of the antibody) was added to deplete the excess payload, EDTA (100 −200 mM, 4.0-6.0 eq. of the antibody) was added to trap zinc, and DHIAA (100 −200 mM, 8.0-30.0 eq. of the antibody) was added to oxidize the free thiol groups in the protein. The reaction mixture was finally purified using a de-salting column (Zeba Spin Desalting Columns, 40K MWCO), or UF/DF, or ion exchange chromatography, and drug/antibody ratio (DAR) were analyzed using HIC-HPLC or HPLC-MS. The HIC-HPLC results are exampled in Table 1 and 2.

### Example 469. DALR Analysis

DALR was analyzed by using HIC-HPLC, and the HPLC parameters are as follows:

| HPLC | Agilent 1260 | | | | | |
|---|---|---|---|---|---|---|
| Column | Thermo HIC butyl 4.6 × 100 mm | | | | | |
| Phase A | PBS buffer containing (NH₄)₂SO₄, | | | | | |
| Phase B | PBS buffer | | | | | |
| Sample | injection volume 10 μL | | | | | |
| Rate | 0.8 mL/min | | | | | |
| Wavelength | 280 nm | | | | | |
| Gradient | Time (min) | 0 | 35 | 40 | 42 | 48 |
| | Phase A (%) | 80 | 0 | 0 | 80 | 80 |
| | Phase B (%) | 20 | 100 | 100 | 20 | 20 |

TABLE 1
Drug distribution of BCMA-ADCs (C-408b) analyzed
by HIC-HPLC for the conjugation condition at 3.6
eq. of TCEP with different equivalents of Zinc complexes,
pH 7.2, in 13-16 h of conjugation at 4° C.:

| | Eq. of Zinc | | | | | | |
|---|---|---|---|---|---|---|---|
| Zinc complex | complex | DAR | D 0 | D 2 | D 4 | D 6 | D 8 |
| ZnCl₂ | 2.0 | 4.51 | 0.88 | 10.96 | 64.81 | 8.48 | 14.87 |
| | 2.2 | 4.47 | 0.92 | 11.72 | 64.48 | 8.62 | 14.26 |
| Z-01 | 1.8 | 4.69 | 0.77 | 5.92 | 69.24 | 5.99 | 18.08 |
| | 2.0 | 4.59 | 0.23 | 8.02 | 69.77 | 6.25 | 15.74 |
| | 2.2 | 4.67 | 0.19 | 7.50 | 68.42 | 6.36 | 17.53 |
| | 2.4 | 4.77 | 0.29 | 6.68 | 66.59 | 7.02 | 19.42 |
| Z-02 | 2.0 | 4.72 | 0.14 | 6.74 | 68.57 | 6.37 | 18.19 |
| | 2.2 | 4.72 | 0.18 | 6.99 | 68.09 | 6.25 | 18.49 |
| | 2.4 | 4.59 | 0.21 | 7.73 | 70.31 | 5.81 | 15.93 |
| | 2.6 | 4.63 | 0.25 | 7.61 | 69.12 | 6.33 | 16.69 |
| Z-03 | 1.8 | 4.39 | 0.30 | 10.79 | 70.67 | 5.51 | 12.72 |
| | 2.0 | 4.36 | 0.38 | 11.63 | 69.85 | 6.04 | 12.11 |
| | 2.2 | 4.35 | 0.37 | 11.31 | 70.59 | 5.99 | 11.74 |
| | 2.4 | 4.18 | 0.64 | 15.12 | 68.37 | 6.13 | 9.74 |
| Z-04 | 1.8 | 4.52 | 0.42 | 7.77 | 71.79 | 5.37 | 14.64 |
| | 2.0 | 4.47 | 1.17 | 6.80 | 73.44 | 4.69 | 13.90 |
| | 2.2 | 4.47 | 0.89 | 7.28 | 72.90 | 5.13 | 13.79 |
| | 2.4 | 4.52 | 0.21 | 7.82 | 72.12 | 5.45 | 14.40 |
| Z-05 | 1.8 | 4.56 | 0.68 | 8.08 | 69.20 | 6.67 | 15.37 |
| | 2.0 | 4.54 | 0.66 | 8.21 | 68.76 | 8.12 | 14.25 |
| | 2.2 | 4.53 | 0.72 | 8.43 | 68.45 | 8.41 | 13.99 |
| Z-06 | 2.0 | 4.46 | 0.40 | 8.31 | 72.43 | 5.69 | 13.17 |
| | 2.2 | 4.42 | 0.27 | 8.09 | 74.11 | 5.34 | 12.19 |
| | 2.4 | 4.37 | 0.59 | 8.02 | 74.73 | 5.44 | 11.22 |
| | 2.6 | 4.38 | 0.60 | 8.12 | 74.02 | 5.78 | 11.48 |
| Z-07 | 2.0 | 4.65 | 0.23 | 7.33 | 69.18 | 6.46 | 16.80 |
| | 2.2 | 4.63 | 0.17 | 6.93 | 70.46 | 6.13 | 16.31 |
| | 2.4 | 4.57 | 0.63 | 5.73 | 73.04 | 5.61 | 14.99 |
| | 2.6 | 4.50 | 0.60 | 7.03 | 72.33 | 6.39 | 13.65 |
| Z-08 | 2.0 | 4.52 | 1.20 | 6.85 | 71.59 | 5.59 | 14.77 |
| | 2.2 | 4.56 | 0.34 | 8.25 | 69.99 | 5.85 | 15.57 |
| | 2.4 | 4.49 | 0.76 | 7.40 | 72.34 | 5.39 | 14.12 |
| | 2.6 | 4.49 | 0.71 | 7.92 | 71.43 | 5.72 | 14.22 |
| Z-09 | 2.0 | 4.68 | 0.82 | 6.17 | 68.58 | 6.81 | 17.62 |
| | 2.2 | 4.66 | 1.30 | 5.48 | 69.56 | 6.24 | 17.42 |
| | 2.4 | 4.65 | 0.22 | 6.88 | 69.97 | 5.84 | 17.10 |
| | 2.6 | 4.64 | 0.71 | 6.67 | 69.51 | 6.10 | 17.01 |
| Z-10 | 2.0 | 4.55 | 0.86 | 7.74 | 70.30 | 5.32 | 15.78 |
| | 2.2 | 4.50 | 1.41 | 6.86 | 71.75 | 5.47 | 14.51 |
| | 2.4 | 4.41 | 1.08 | 9.08 | 71.30 | 5.46 | 13.08 |
| | 2.6 | 4.45 | 0.98 | 8.61 | 71.01 | 5.42 | 13.98 |
| Z-11 | 2.0 | 4.56 | 0.29 | 7.58 | 70.84 | 6.49 | 14.81 |
| | 2.2 | 4.50 | 0.58 | 6.71 | 73.13 | 6.24 | 13.34 |
| | 2.4 | 4.48 | 0.14 | 6.19 | 75.46 | 5.86 | 12.35 |
| | 2.6 | 4.49 | 0.31 | 6.65 | 74.35 | 5.81 | 12.88 |
| Z-13 | 1.8 | 4.52 | 0 | 8.37 | 70.66 | 7.47 | 13.50 |
| | 2.0 | 4.54 | 0 | 6.35 | 73.92 | 5.98 | 13.76 |
| | 2.2 | 4.44 | 0 | 7.66 | 74.76 | 5.71 | 11.87 |
| | 2.4 | 4.40 | 0.08 | 8.18 | 74.89 | 5.43 | 11.42 |
| | 2.6 | 4.42 | 0.10 | 7.99 | 74.52 | 5.67 | 11.72 |
| Z-14 | 1.8 | 4.48 | 0 | 8.63 | 72.34 | 5.60 | 13.42 |
| | 2.0 | 4.45 | 0 | 7.71 | 74.46 | 5.48 | 12.35 |
| | 2.2 | 4.41 | 0 | 8.88 | 73.72 | 5.55 | 11.85 |
| | 2.4 | 4.39 | 0 | 9.35 | 73.64 | 5.40 | 11.62 |
| | 2′6 | 4.39 | 0.08 | 9.11 | 73.48 | 5.63 | 11.70 |
| Z-15 | 1.8 | 4.61 | 0 | 6.62 | 71.87 | 5.69 | 15.82 |
| | 2.0 | 4.48 | 0.98 | 7.67 | 71.47 | 5.96 | 13.92 |
| | 2.2 | 4.46 | 0 | 8.44 | 73.09 | 5.36 | 13.11 |
| | 2.4 | 4.43 | 0 | 8.07 | 74.56 | 5.38 | 11.99 |
| | 2.6 | 4.45 | 0.11 | 8.11 | 73.98 | 5.48 | 12.32 |
| Z-16 | 1.8 | 4.46 | 0 | 8.59 | 72.73 | 5.74 | 12.94 |
| | 2.0 | 4.41 | 0 | 9.47 | 72.69 | 5.91 | 11.93 |
| | 2.2 | 4.35 | 0 | 10.68 | 72.33 | 5.64 | 11.35 |
| | 2.4 | 4.42 | 0 | 7.70 | 75.35 | 5.88 | 11.07 |
| | 2.6 | 4.35 | 0.08 | 8.91 | 74.82 | 5.71 | 10.48 |
| Z-17 | 1.8 | 4.46 | 0 | 8.44 | 72.72 | 6.03 | 12.81 |
| | 2.0 | 4.41 | 0 | 10.48 | 70.86 | 6.23 | 12.43 |
| | 2.2 | 4.44 | 0 | 8.89 | 72.57 | 6.08 | 12.46 |
| | 2.4 | 4.42 | 0 | 8.06 | 74.59 | 5.74 | 11.61 |
| | 2.6 | 4.43 | 0.07 | 8.11 | 73.82 | 5.98 | 12.02 |
| Z-19 | 2.0 | 4.18 | 0.51 | 12.52 | 73.42 | 4.74 | 8.80 |
| | 2.2 | 4.20 | 0.52 | 12.92 | 71.90 | 5.29 | 9.37 |
| Z-20 | 2.0 | 4.32 | 0.36 | 10.17 | 73.54 | 5.01 | 10.91 |
| | 2.2 | 4.29 | 0.38 | 10.75 | 73.19 | 5.16 | 10.51 |
| Z-21 | 2.0 | 4.42 | 0.39 | 7.84 | 74.76 | 4.57 | 12.44 |
| | 2.2 | 4.33 | 0.40 | 8.34 | 76.09 | 4.68 | 10.49 |
| | 2.4 | 4.34 | 0.38 | 8.38 | 75.78 | 4.59 | 10.87 |
| Z-22 | 2.0 | 4.42 | 0.36 | 9.99 | 71.29 | 5.13 | 13.23 |
| | 2.2 | 4.41 | 0.34 | 8.14 | 74.38 | 4.99 | 12.14 |
| | 2.4 | 4.40 | 0.41 | 8.49 | 73.82 | 5.07 | 12.21 |
| Z-23 | 2.0 | 4.49 | 0.16 | 8.10 | 72.55 | 5.27 | 13.92 |
| | 2.2 | 4.41 | 0.15 | 8.42 | 74.25 | 5.02 | 12.16 |
| | 2.4 | 4.42 | 0.21 | 8.40 | 73.79 | 5.11 | 12.49 |
| Z-25 | 2.2 | 4.26 | 0.54 | 11.43 | 72.87 | 5.03 | 10.13 |
| Z-26 | 2.0 | 4.48 | 0.41 | 9.25 | 70.44 | 5.65 | 14.25 |
| | 2.2 | 4.29 | 0.43 | 10.32 | 73.86 | 4.86 | 10.53 |
| | 2.4 | 4.32 | 0.62 | 10.01 | 73.11 | 4.92 | 11.34 |
| Z-27 | 2 | 4.39 | 0.36 | 8.62 | 74.18 | 4.96 | 11.88 |
| | 2.2 | 4.37 | 0.32 | 9.26 | 73.80 | 5.07 | 11.55 |
| | 2.4 | 4.36 | 0.41 | 9.41 | 73.53 | 5.05 | 11.60 |
| Z-28 | 2.0 | 4.39 | 0.36 | 8.02 | 75.08 | 4.96 | 11.59 |
| | 2.2 | 4.31 | 0.43 | 9.24 | 75.20 | 4.57 | 10.56 |
| | 2.4 | 4.32 | 0.47 | 9.01 | 74.90 | 5.09 | 10.53 |
| Z-29 | 2.0 | 4.08 | 6.50 | 8.07 | 70.53 | 4.50 | 10.40 |
| | 2.2 | 4.04 | 6.93 | 8.14 | 70.70 | 4.34 | 9.89 |
| | 2.4 | 4.07 | 6.41 | 8.49 | 70.11 | 4.77 | 10.22 |
| Z-31 | 1.5 | 4.94 | 0 | 1.75 | 69.81 | 8.26 | 20.19 |
| | 1.8 | 4.89 | 0 | 1.53 | 71.70 | 7.61 | 19.16 |
| | 2.0 | 4.87 | 0 | 1.45 | 72.38 | 7.18 | 18.99 |
| | 2.2 | 4.78 | 0 | 2.65 | 73.24 | 6.64 | 17.47 |
| | 2.4 | 4.83 | 0 | 1.56 | 73.46 | 6.98 | 18.01 |
| | 2.6 | 4.82 | 0 | 1.83 | 73.18 | 6.82 | 18.17 |
| Z-32 | 1.5 | 4.85 | 0 | 1.49 | 72.98 | 7.00 | 18.52 |
| | 1.8 | 4.80 | 0 | 1.42 | 74.04 | 7.53 | 17.01 |
| | 2.2 | 4.71 | 0 | 1.32 | 77.34 | 5.98 | 15.36 |
| | 2.4 | 4.65 | 0 | 1.66 | 78.21 | 5.91 | 14.22 |
| | 2.6 | 4.69 | 0 | 2.07 | 76.60 | 5.95 | 15.37 |
| Z-33 | 1.5 | 4.9 | 0 | 1.59 | 69.64 | 10.82 | 17.95 |
| | 2.0 | 4.93 | 0 | 1.90 | 68.33 | 11.12 | 18.65 |
| | 2.2 | 4.96 | 0 | 1.27 | 67.92 | 12.14 | 18.67 |
| | 2.4 | 5.04 | 0 | 0.90 | 66.62 | 11.78 | 20.70 |
| | 2.6 | 5.08 | 0 | 0.86 | 65.16 | 12.90 | 21.08 |

TABLE 2
Drug distribution of BCMA-ADCs (C-408b) analyzed by HIC-HPLC
for the conjugation condition at 3.0-4.0 eq. of TCEP, 2.2 or
2.4 equivalents of Z-11, Z-16, Z-21, Z-28 and Z-32, 6.0 eq
of compound 408b, pH 7.0, in 15 h of conjugation at 4° C.

| Zn | Eq. | | | | | | | |
|---|---|---|---|---|---|---|---|---|
| amino | of | Eq. | | | | | | |
| Com- | com- | of | | D | D | D | D | D |
| plex | plex | TCEP | DAR | 0 | 2 | 4 | 6 | 8 |
| Z-11 | 2.2 | 3.0 | 3.8 | 1.10 | 17.37 | 73.57 | 5.39 | 2.57 |
| Z-11 | 2.2 | 3.2 | 3.9 | 1.04 | 13.1 | 77.47 | 5.37 | 3.02 |
| Z-11 | 2.2 | 3.4 | 4.0 | 0.04 | 10.84 | 79.51 | 5.78 | 3.83 |
| Z-11 | 2.2 | 3.6 | 4.2 | 0.03 | 7.03 | 80.43 | 6.05 | 6.46 |
| Z-11 | 2.2 | 3.8 | 4.4 | 0.09 | 4.32 | 80.82 | 5.24 | 9.53 |
| Z-11 | 2.2 | 4.0 | 4.4 | 0.02 | 3.57 | 80.57 | 4.92 | 10.92 |
| Z-11 | 2.4 | 3.0 | 4.0 | 0.32 | 12.88 | 77.57 | 5.21 | 4.02 |
| Z-11 | 2.4 | 3.2 | 4.0 | 0.22 | 10.40 | 79.57 | 5.20 | 4.61 |
| Z-11 | 2.4 | 3.4 | 4.2 | 0.10 | 7.59 | 80.81 | 5.48 | 6.02 |
| Z-11 | 2.4 | 3.6 | 4.1 | 0.21 | 8.06 | 81.08 | 5.04 | 5.61 |
| Z-11 | 2.4 | 3.8 | 4.3 | 0.19 | 5.87 | 80.63 | 6.14 | 7.17 |
| Z-11 | 2.4 | 4.0 | 4.3 | 0.29 | 6.39 | 79.98 | 4.16 | 9.18 |
| Z-16 | 2.4 | 3.2 | 3.9 | 1.50 | 14.46 | 75.54 | 4.50 | 4.00 |
| Z-16 | 2.4 | 3.4 | 4.0 | 0.08 | 11.71 | 78.57 | 5.38 | 4.26 |
| Z-16 | 2.4 | 3.6 | 4.0 | 0.35 | 11.53 | 77.7 | 6.13 | 4.29 |
| Z-16 | 2.4 | 3.8 | 4.1 | 0.14 | 10.32 | 77.58 | 6.05 | 6.91 |
| Z-21 | 2.4 | 3.2 | 4.0 | 0.10 | 10.58 | 78.97 | 6.34 | 4.01 |
| Z-21 | 2.4 | 3.4 | 4.1 | 0.15 | 8.48 | 80.46 | 5.87 | 5.04 |
| Z-21 | 2.4 | 3.6 | 4.3 | 0.08 | 5.15 | 81.05 | 4.68 | 9.04 |
| Z-21 | 2.4 | 3.8 | 4.3 | 0.15 | 5.22 | 80.84 | 4.62 | 9.17 |
| Z-28 | 2.4 | 3.0 | 4.0 | 0.34 | 11.32 | 79.74 | 4.56 | 4.04 |
| Z-28 | 2.4 | 3.2 | 4.1 | 0.19 | 9.2 | 81.12 | 4.75 | 4.74 |
| Z-28 | 2.4 | 3.4 | 4.2 | 0.02 | 6.78 | 83.15 | 4.68 | 5.37 |
| Z-28 | 2.4 | 3.6 | 4.3 | 0.29 | 4.58 | 81.82 | 4.27 | 9.04 |
| Z-28 | 2.4 | 3.8 | 4.4 | 0.07 | 4.15 | 80.94 | 4.23 | 10.61 |
| Z-28 | 2.4 | 4.0 | 4.4 | 0.03 | 4.99 | 80.03 | 4.16 | 10.79 |
| Z-32 | 2.4 | 3.0 | 4.0 | 0.34 | 11.32 | 79.74 | 4.56 | 4.04 |
| Z-32 | 2.4 | 3.2 | 4.1 | 0.16 | 9.01 | 81.11 | 5.71 | 4.01 |
| Z-32 | 2.4 | 3.4 | 4.2 | 0.12 | 6.68 | 82.17 | 5.18 | 5.85 |
| Z-32 | 2.4 | 3.6 | 4.3 | 0.19 | 4.63 | 81.82 | 4.29 | 9.07 |
| Z-32 | 2.4 | 3.8 | 4.4 | 0.07 | 4.15 | 80.94 | 4.22 | 10.62 |
| Z-32 | 2.4 | 4.0 | 4.4 | 0.03 | 4.99 | 80.03 | 4.17 | 10.78 |

### Example 470. General Preparation of Formulation of the Conjugates

In a liquid formulation of 80 mg of each conjugate (with the antibody of BCMA, Her2, EGFR, CD33, Trop2, Steap1, CD56, PSMA and Her3): C-009, C-020, C-025, C-027, C-031, C-037, C-038, C-039, C-043, C-046, C-052, C-056, C-059, C-063, C-066, C-071, C-079, C-084, C-087, C-093, C-096, C-102, C-109, C-ill, C-118, C-123, C-133, C-143, C-155, C-168, C-172, C-182, C-186, C-198, C-203, C-208, C-214, C-215, C-216, C-217, C-218, C-226, C-227, C-231, C-237, C-249, C-259, C-260, C-261, C-325, C-326, C-327, C-328, C-329, C-330, C331, C-332, C-333, C-334, C-335, C-353, C-363, C-371, C-378, C-379, C-380, C-381, C-384, C-385, C-386, C-387, C-397, C-400, C-403a, C-403b, C-406, C-408a, C-408b, C-408c, C-408d, C-408e, C-408f, C-410a, C-410b, C-410c, C-412a, C-412b, C-412c, C-412d, C-412e, C-412f, C-413a, C-413b, C-413c, C-413d, C-413e, C-413f, C-416a, C-416b, C-416c, C-416d, C-422a, C-422b, C-422c, C-422d, C-425a, C-425b, C-425c, C-425d, C-428a, C-428b, C-428c, C-428d, C-431a, C-431b, C-431c, C-431d, in the 10 mL of borosilicate vial containing 240 mg of sucrose, 0.8 mg of polysorbate-80, 24 mg of sodium citrate in 4 mL of sterile water were adjusted with citric acid to pH 6.0. Then each of the conjugate solution was lyophilized at temperature from −65° C. to 0° C., and to RT at reduced pressure (5˜10 torr) to form a dryness cake. The cake conjugates were stored at 2˜8° C., and then reconstituted with 4 mL of sterile water for further application.

Example 471. In vitro cytotoxicity evaluation of the conjugate (with the antibody of BCMA Her2, EGFR, Trop2, Steap1, CD56, PSMA and Her3): C-009, C-020, C-025, C-027, C-031, C-037, C-038, C-039, C-043, C-046, C-052, C-056, C-059, C-063, C-066, C-071, C-079, C-084, C-087, C-093, C-096, C-102, C-109, C-111, C-118, C-123, C-133, C-143, C-155, C-168, C-172, C-182, C-186, C-198, C-203, C-208, C-214, C-215, C-216, C-217, C-218, C-226, C-227, C-231, C-237, C-249, C-259, C-260, C-261, C-325, C-326, C-327, C-328, C-329, C-330, C331, C-332, C-333, C-334, C-335, C-353, C-363, C-371, C-378, C-379, C-380, C-381, C-384, C-385, C-386, C-387, C-397, C-400, C-403a, C-403b, C-406, C-408a, C-408b, C-408c, C-408d, C-408e, C-408f, C-410a, C-410b, C-410c, C-412a, C-412b, C-412c, C-412d, C-412e, C-412f, C-413a, C-413b, C-413c, C-413d, C-413e, C-413f, C-416a, C-416b, C-416c, C-416d, C-422a, C-422b, C-422c, C-422d, C-425a, C-425b, C-425c, C-425d, C-428a, C-428b, C-428c, C-428d, C-431a, C-431b, C-431c, C-431d, in comparison with Paclitaxel. For evaluation of Her2-ADCs, the comparison was chosen T-DM1.

The cell lines used in the cytotoxicity assays were (1). Myeloma(+) cells, NCI-H929, and MM1S were obtained from ATCC, and 8226-2A1 cell is Myeloma antigen express cells through culturing and clone-picking of ATCC's RPMI-8226; (2). EGFR (+) cells: HCC-827 is lung cancer cells, and LN229 and U87MG are human glioma tumor cell lines; (3). MUC-1 (+) cells: Colo205 is colon cancer cell line; (4). Trop2 (+) cells: MDA-MB-468 cells is a human triple negative breast cancer cell line, Calu-3 is a Homo sapiens lung adenocarcinoma or a submucosal gland cell line; (5). Her2(+) cells: BT-474 is a human breast cancer cell line, NCI-N87 is a human gastric carcinoma cell line (NCI-N87 cells also express Trop2 antigens); SK-OV-3 is a human ovarian cancer cell line, A431, a human epithelial carcinoma cell line. The cells were grown according to the provider manuals, For instance, N87 cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/mL) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100. The MTT results of BCMA-ADCs are listed in Table 4.

TABLE 3
The Structures of the ADC conjugates of the patent application:

| | |
|---|---|
| 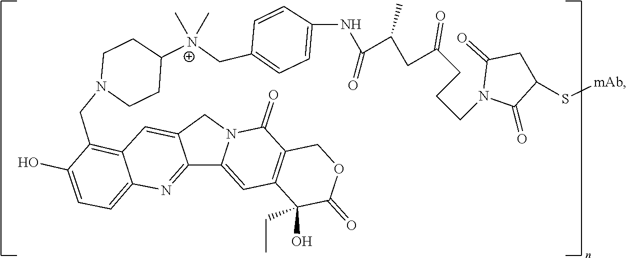 | C-009 |
| 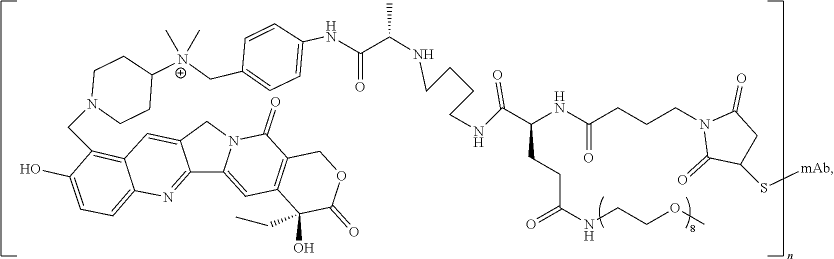 | C-020 |
| 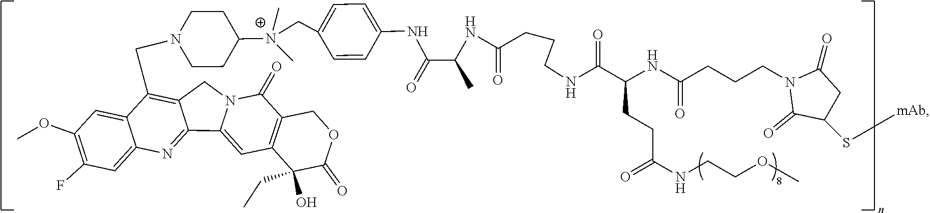 | C-025 |
| 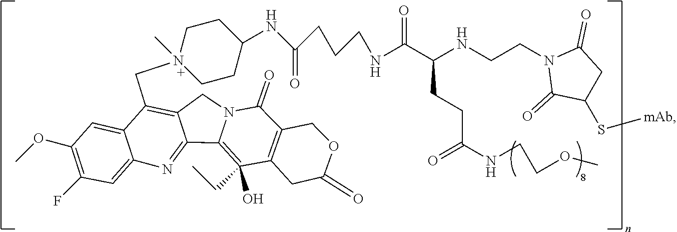 | C-027 |
| 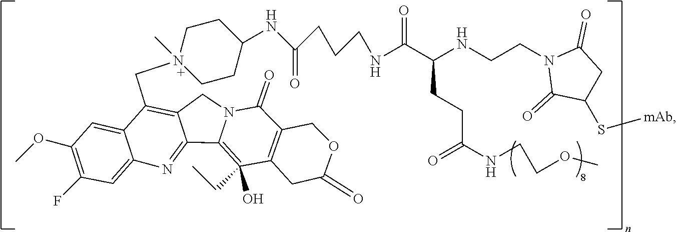 | C-031 |
| 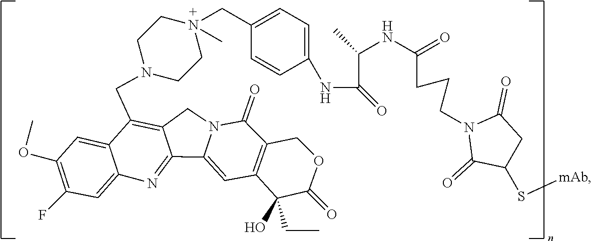 | C-037 |
| 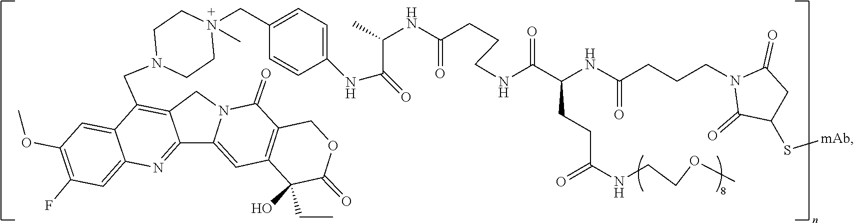 | C-038 |
| 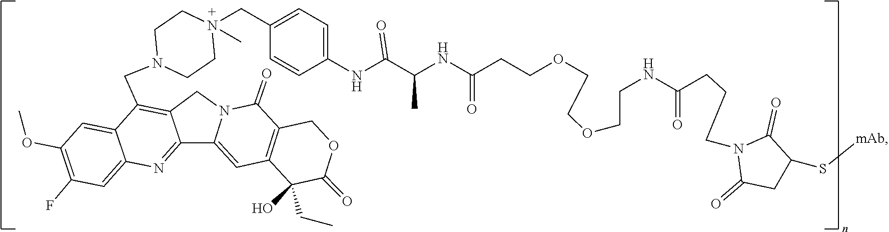 | C-039 |
| 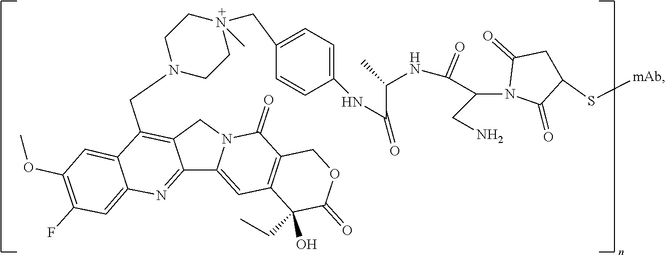 | C-043 |
| 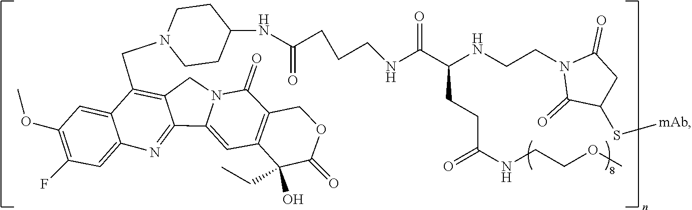 | C-046 |
| 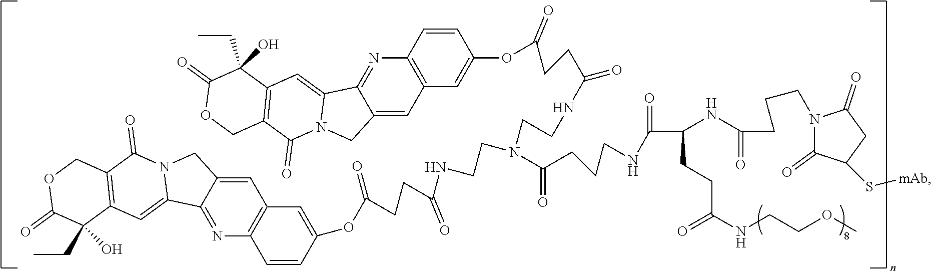 | C-052 |
| 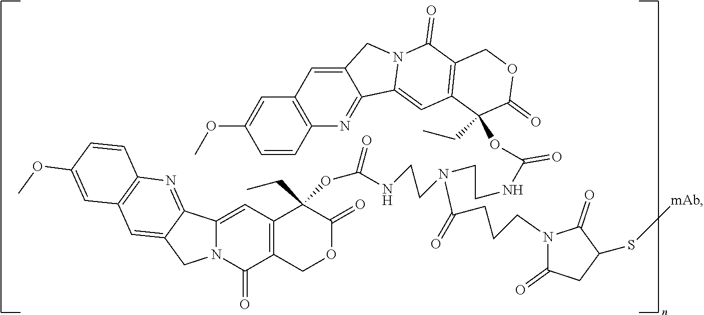 | C-056 |
| 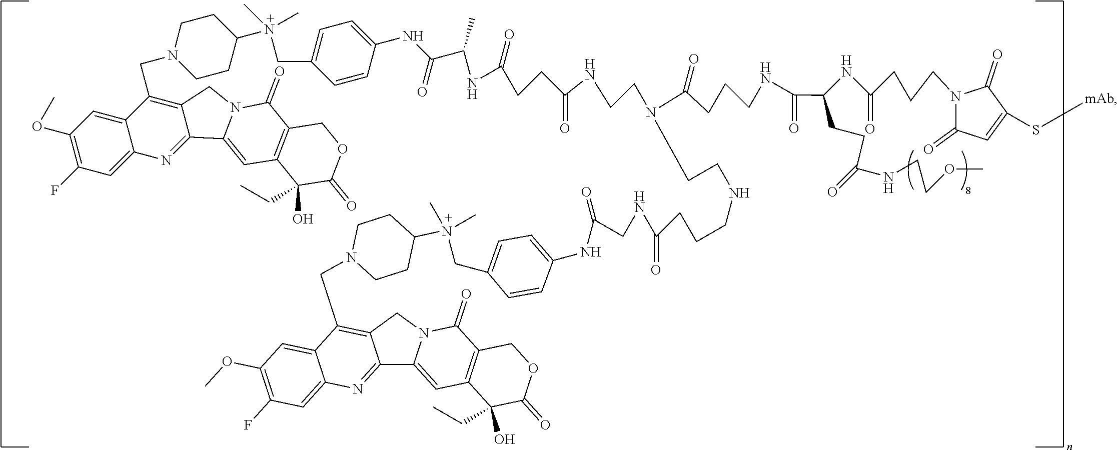 | C-059 |
| 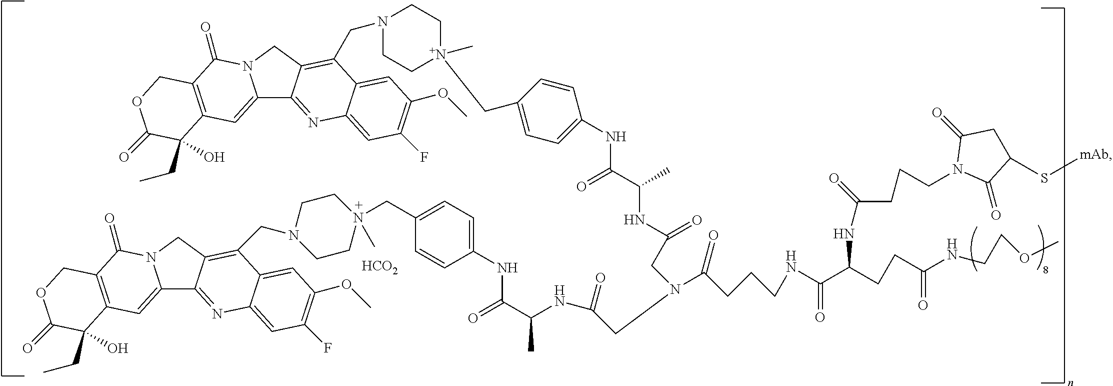 | C-063 |
| 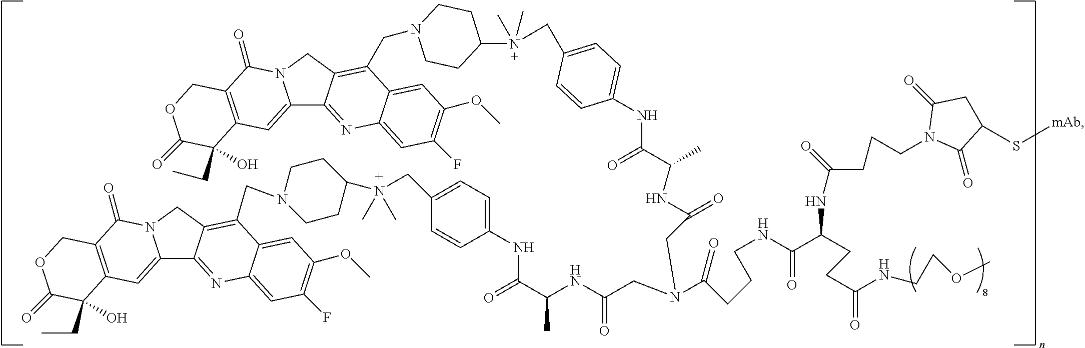 | C-066 |
| 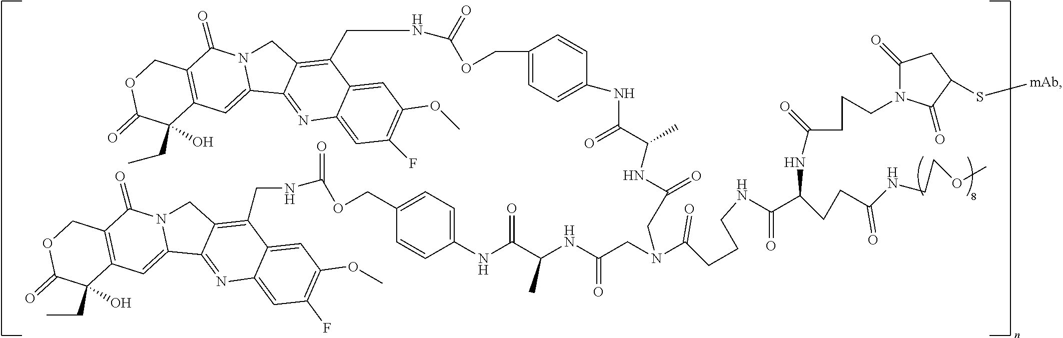 | C-071 |
| 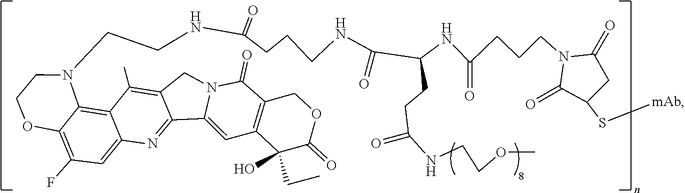 | C-079 |
| 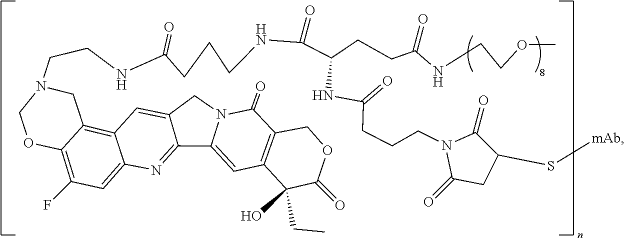 | C-084 |
| 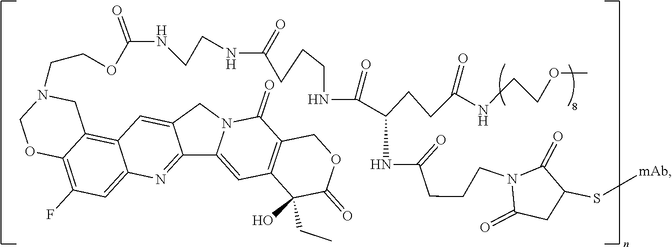 | C-087 |
| 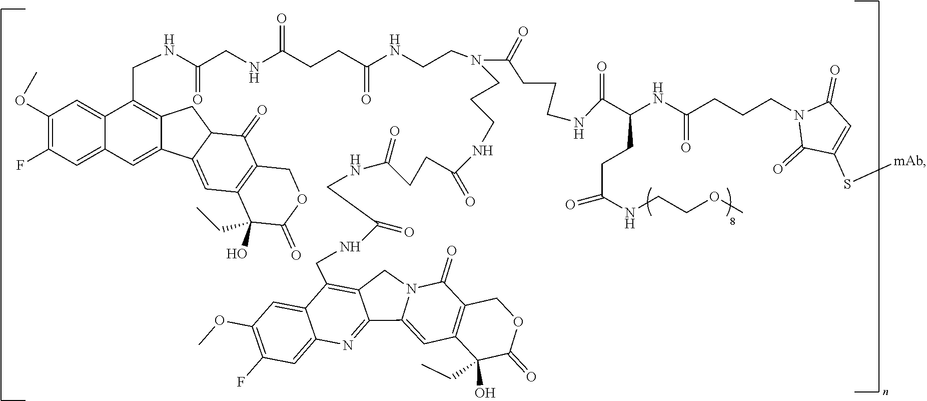 | C-093 |
| 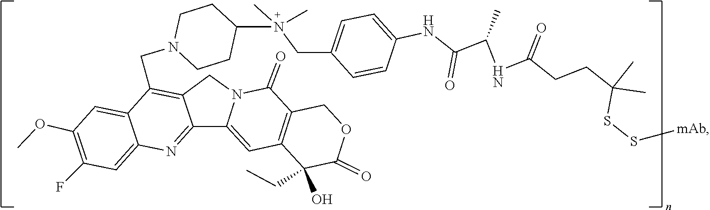 | C-096 |
| 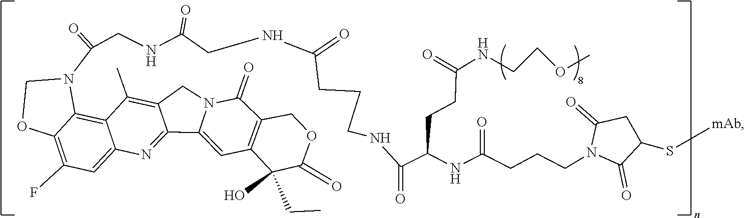 | C-102 |
| 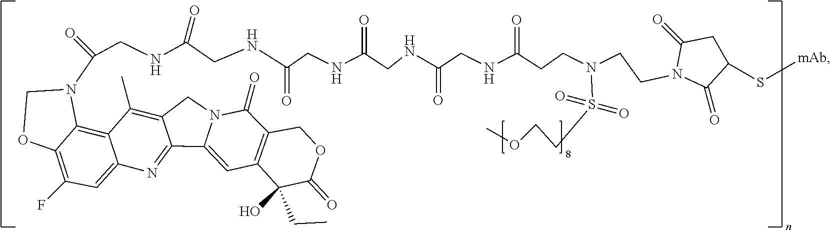 | C-109 |
| 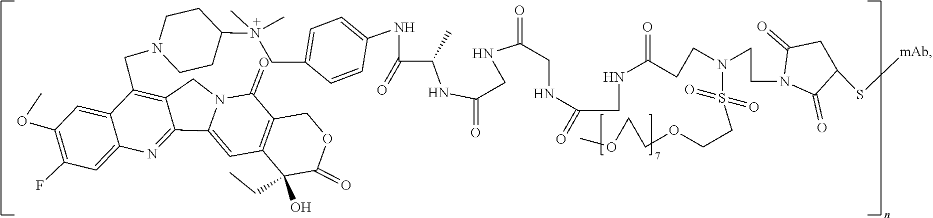 | C-111 |
| 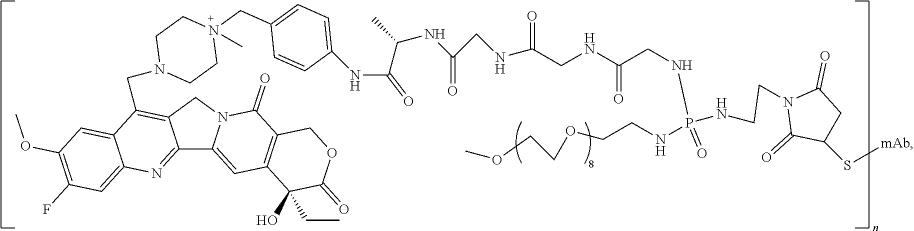 | C-118 |
| 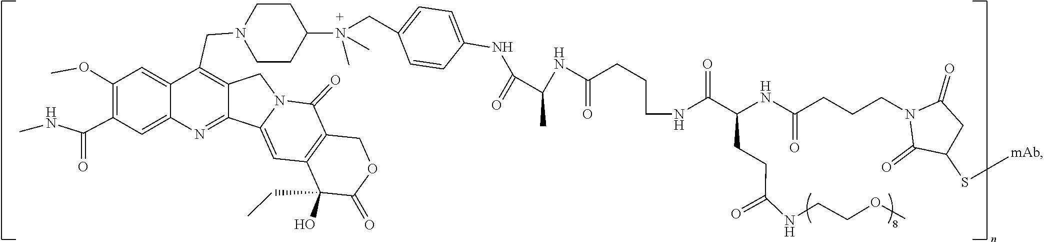 | C-123 |
| 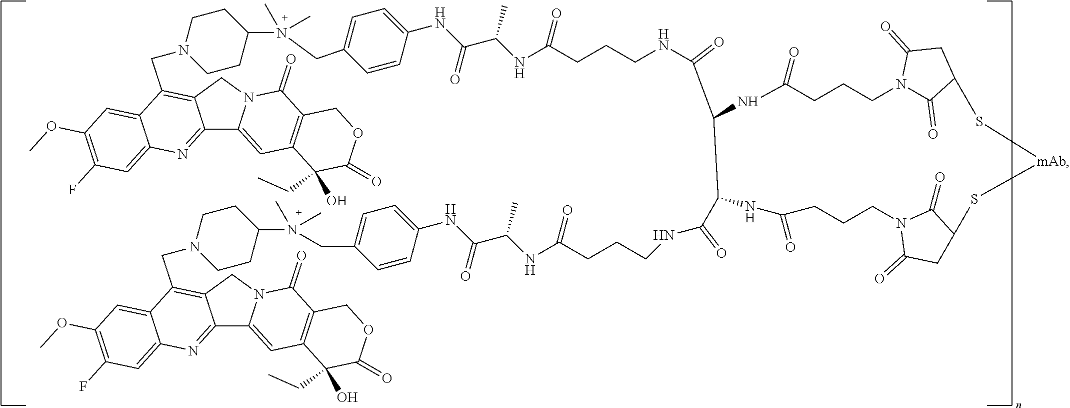 | C-133 |
| 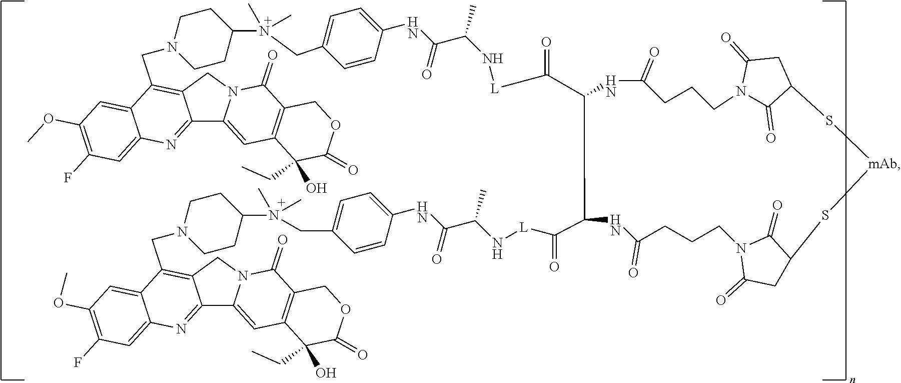 | |
| 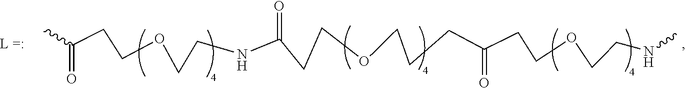 | C-143 |
| 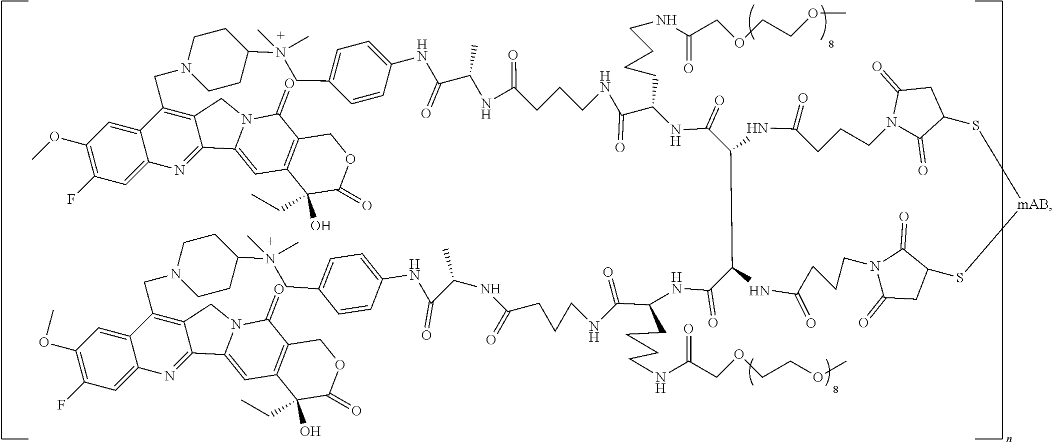 | C-155 |
| 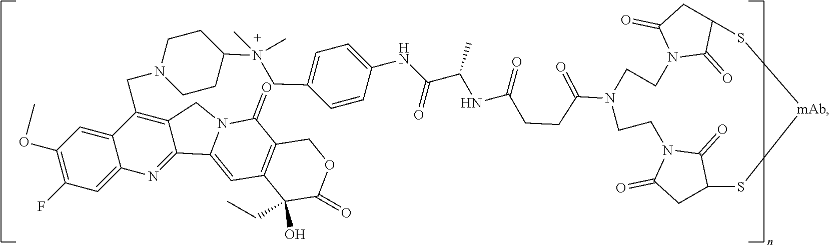 | C-168 |
| 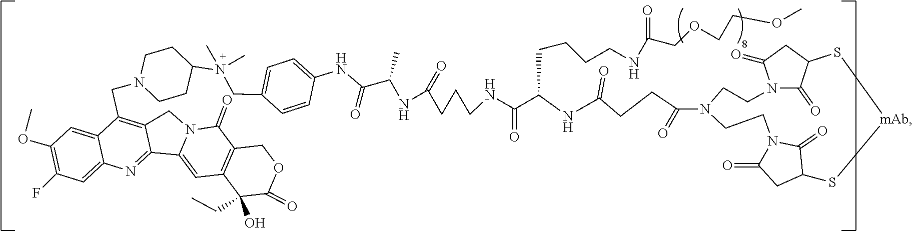 | C-172 |
| 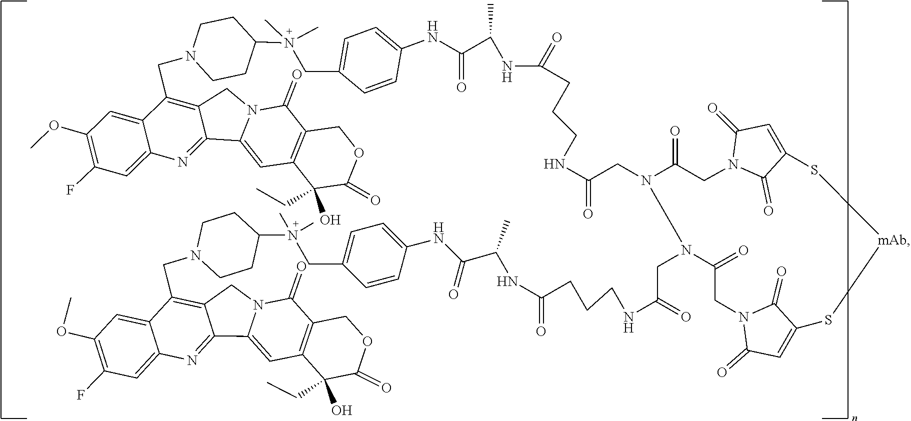 | C-182 |
| 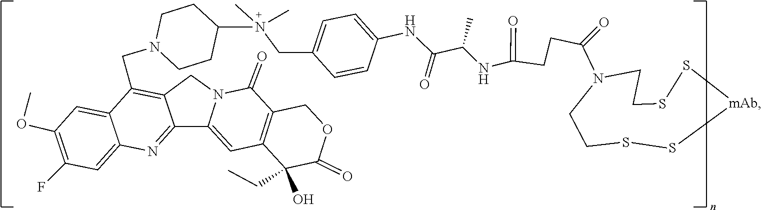 | C-186 |
| 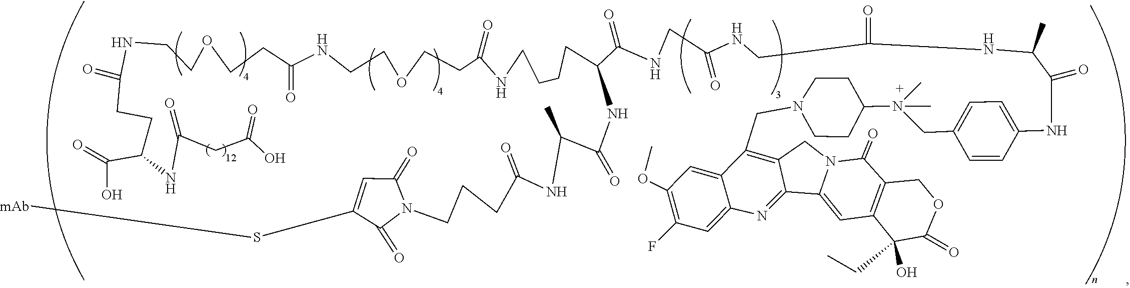 | C-198 |
| 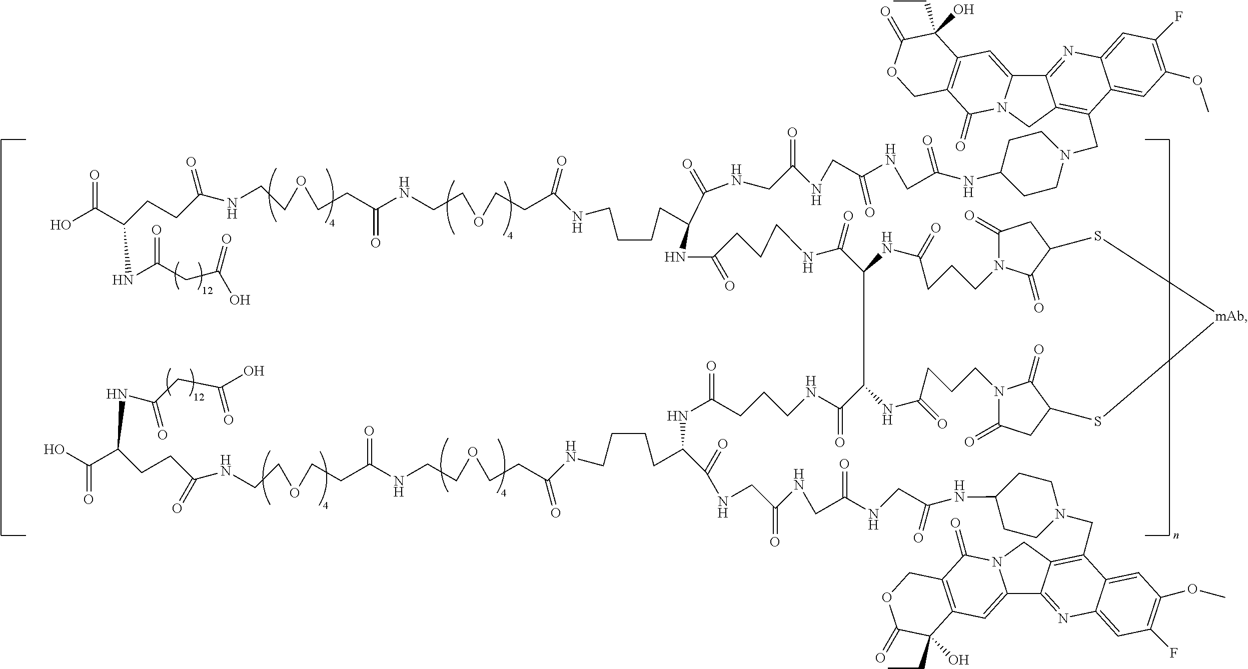 | C-203 |
| 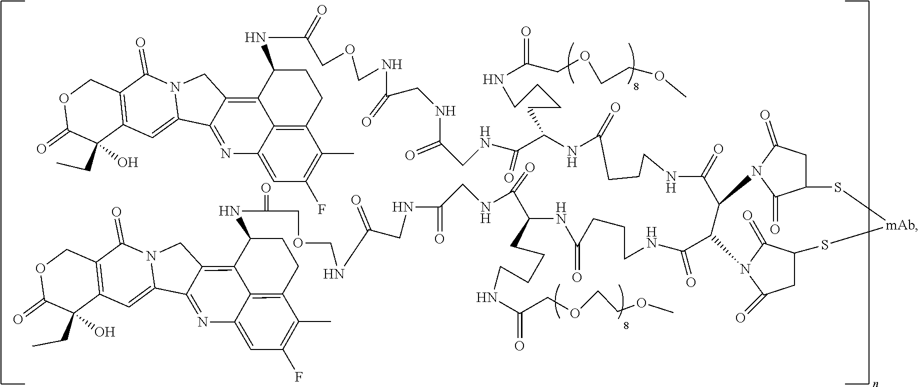 | C-208 |
| 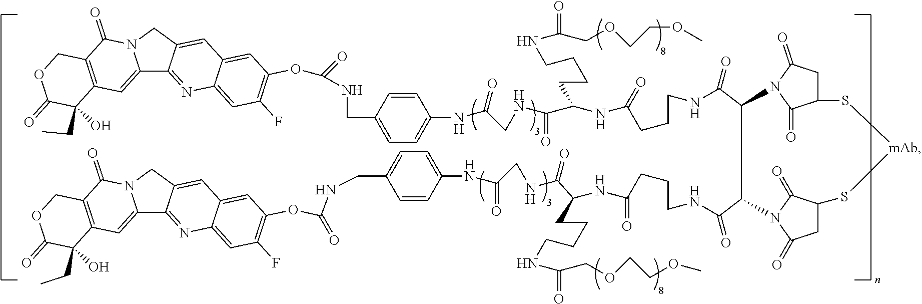 | C-214 |
| 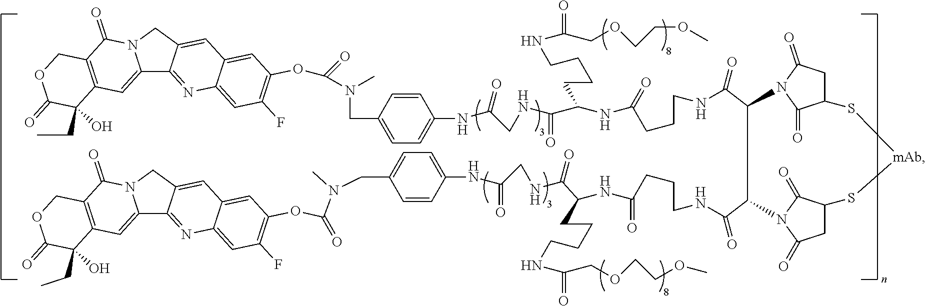 | C-215 |
| 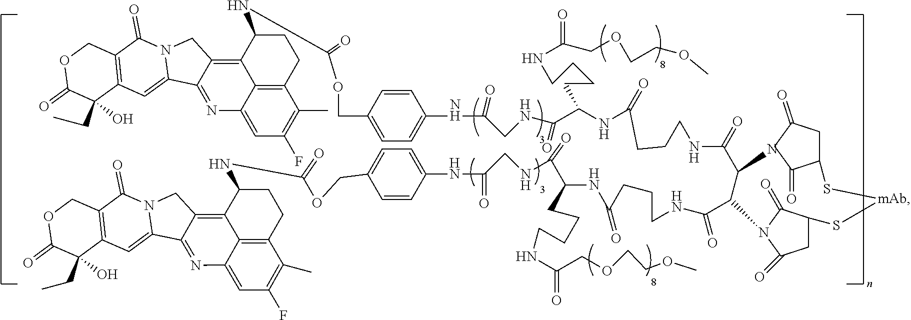 | C-216 |
| 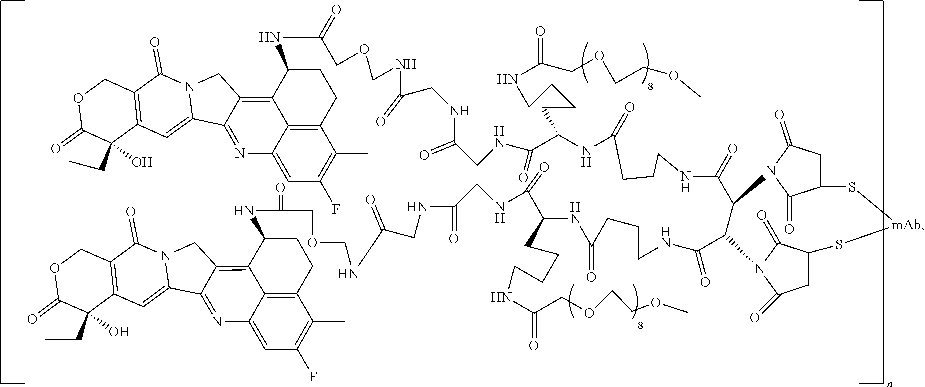 | C-217 |
| 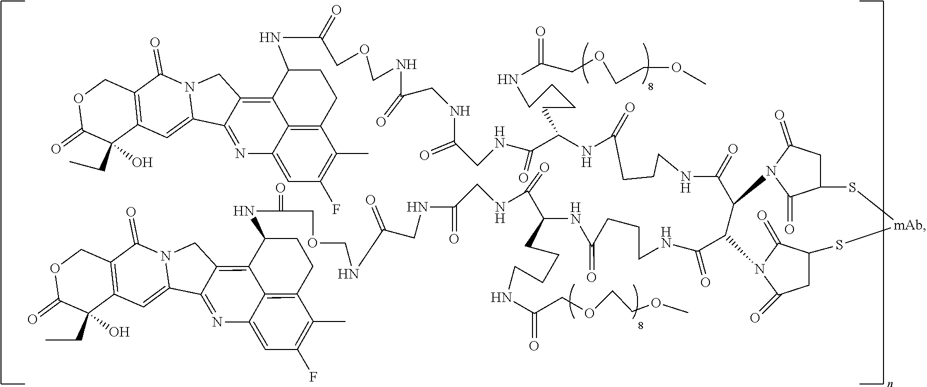 | C-218 |
| 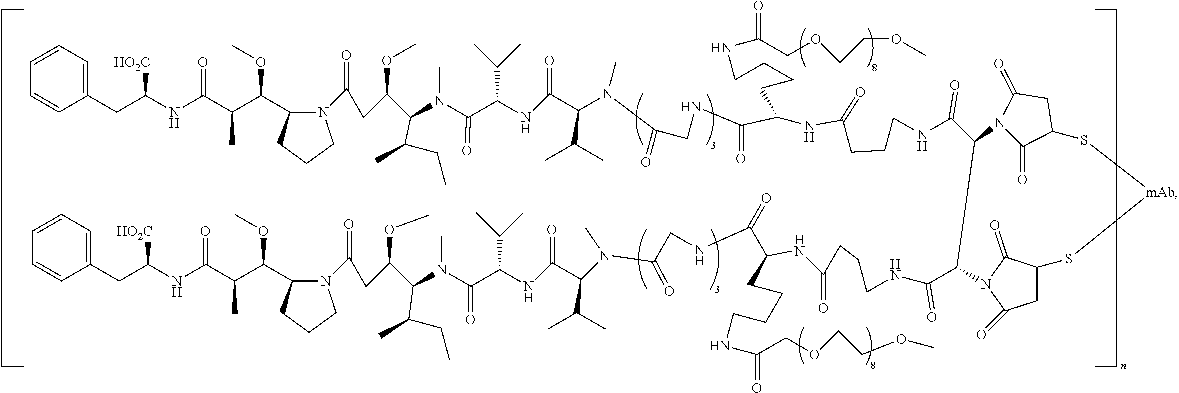 | C-226 |
| 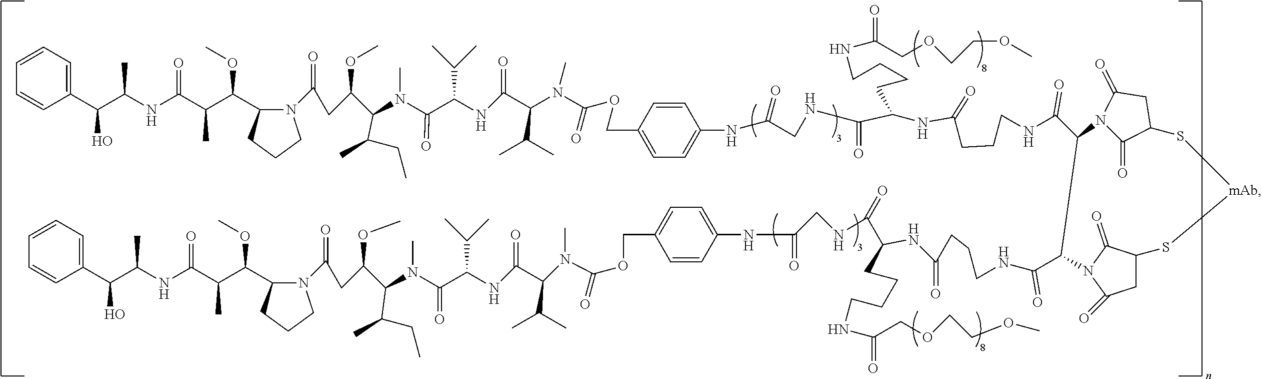 | C-227 |
| 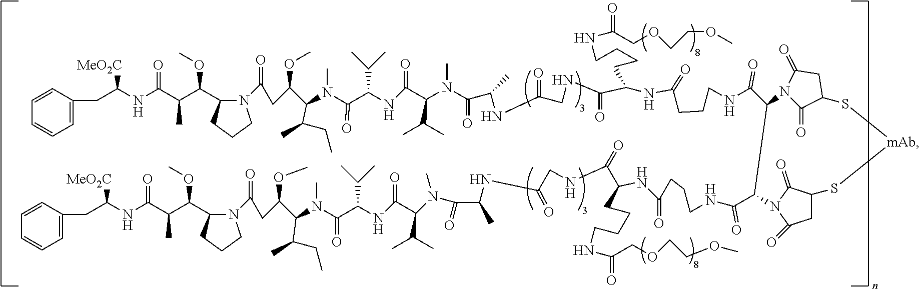 | C-231 |
| 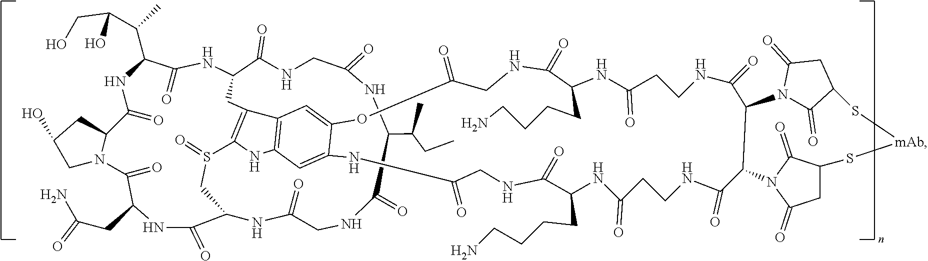 | C-237 |
| 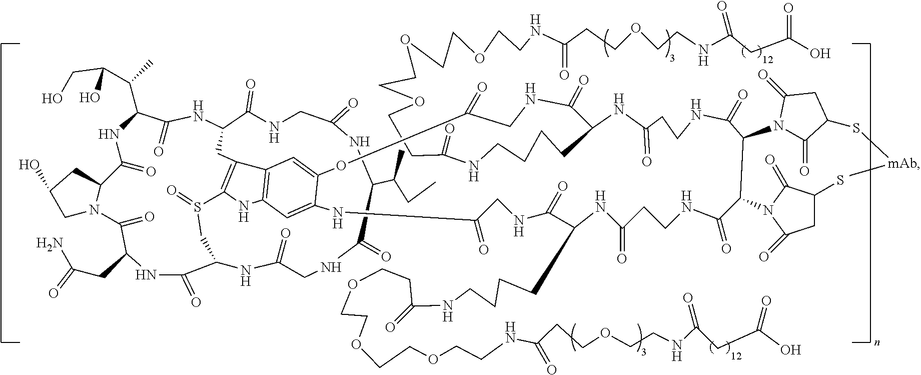 | C-249 |
| 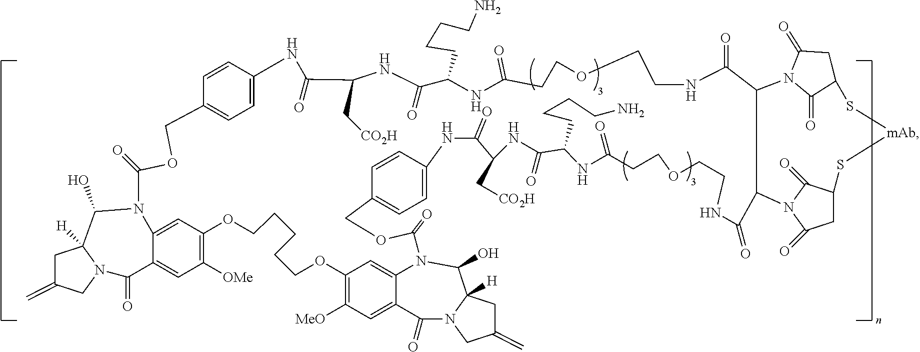 | C-259 |
| 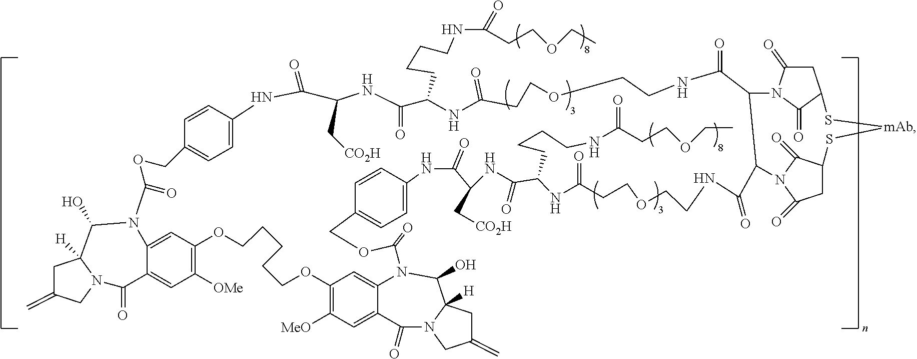 | C-260 |
| 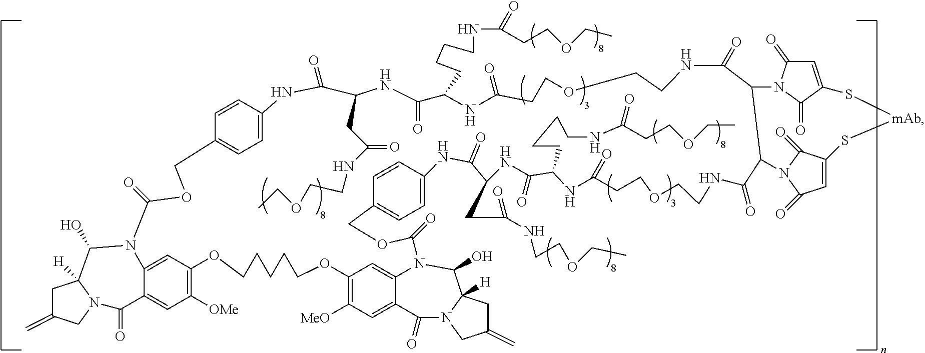 | C-261 |
| 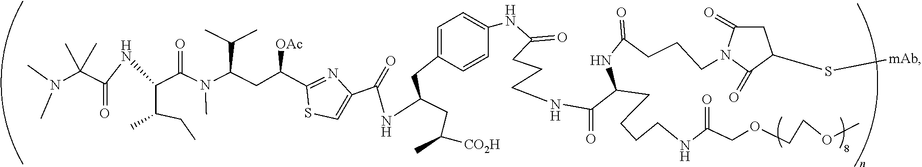 | C-325 |
| 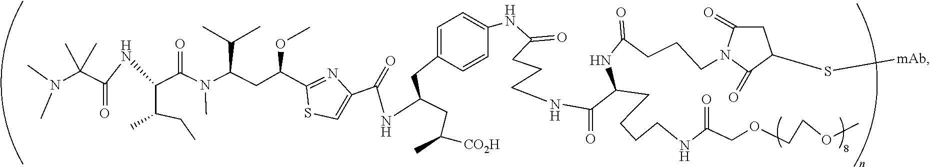 | C-326 |
| 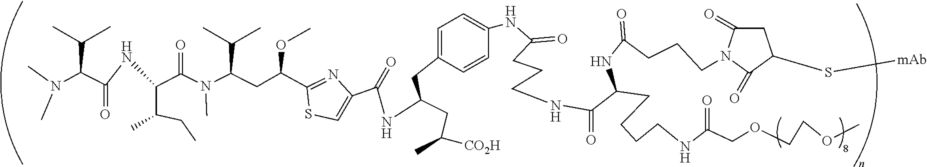 | C-327 |
| 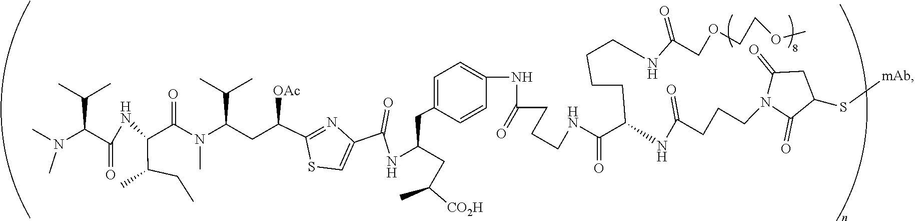 | C-328 |
| 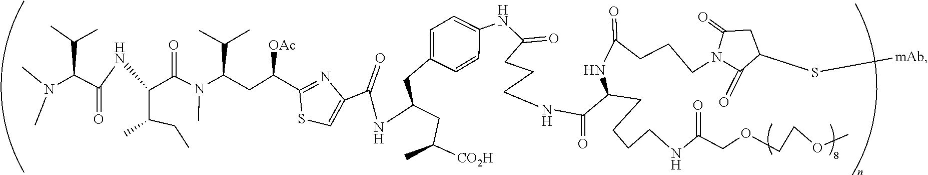 | C-329 |
| 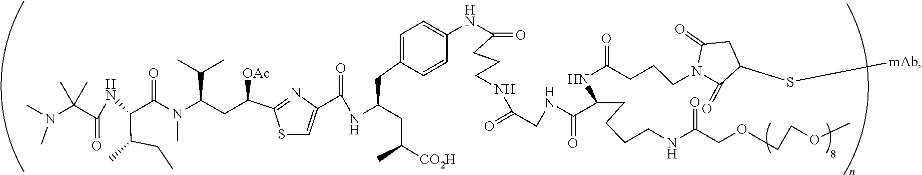 | C-330 |
| 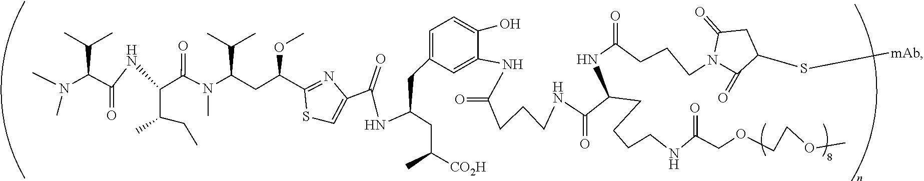 | C-331 |
| 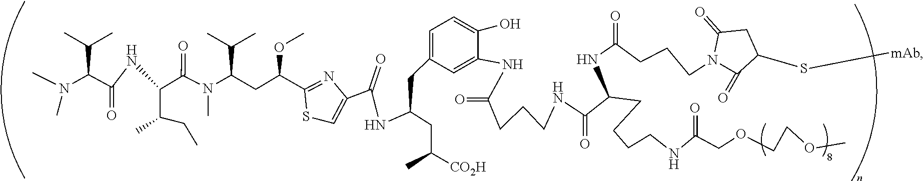 | C-332 |
| 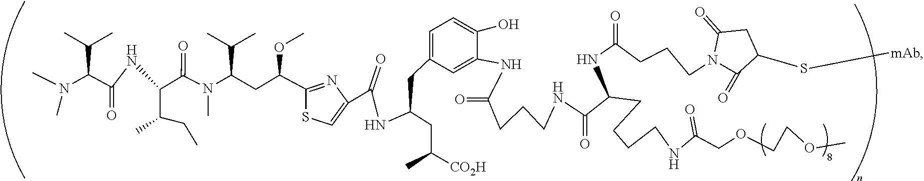 | C-333 |
| 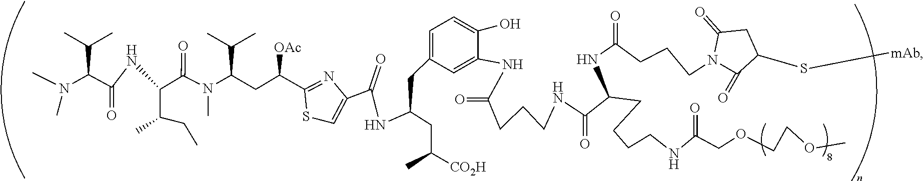 | C-334 |
| 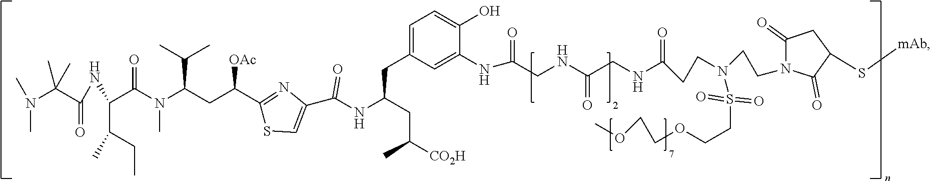 | C-335 |
| 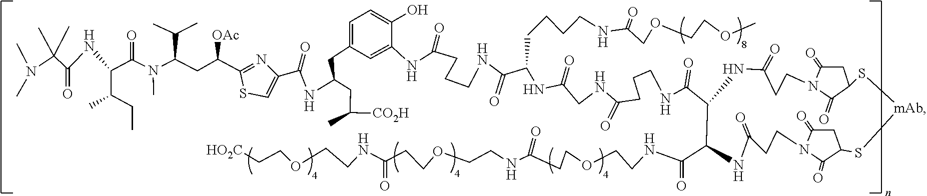 | C-353 |
| 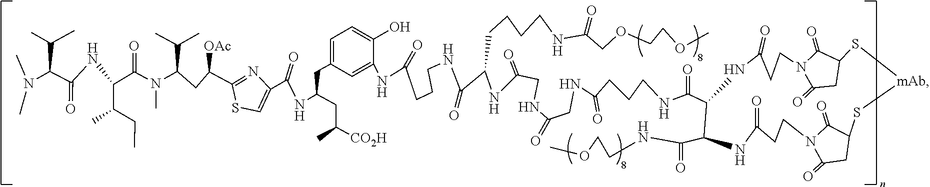 | C-363 |
| 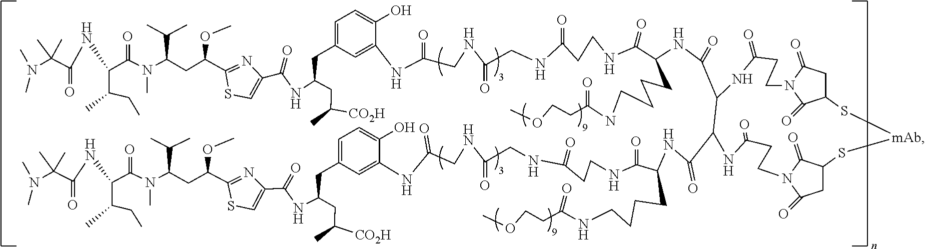 | C-371 |
| 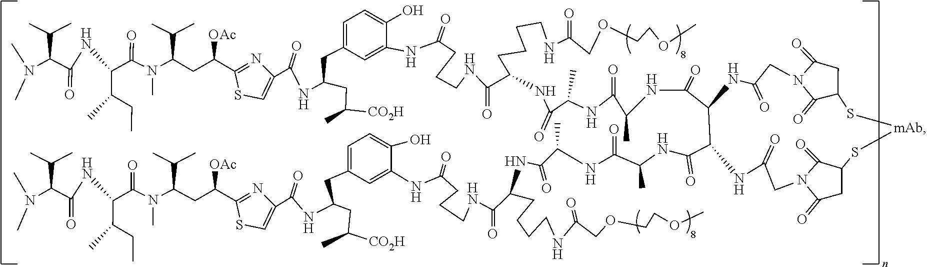 | C-378 |
| 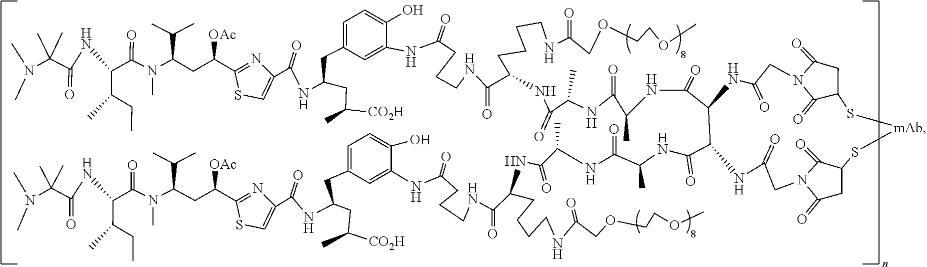 | C-379 |
| 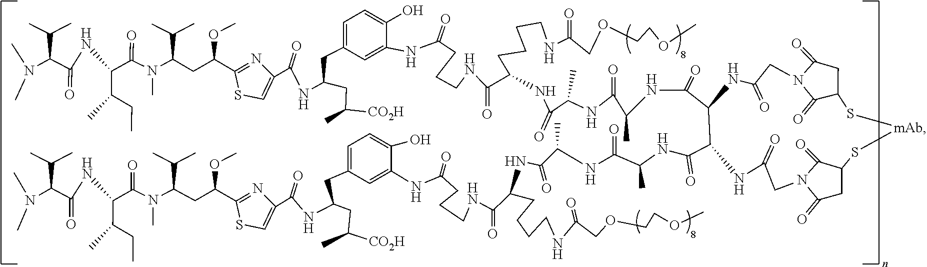 | C-380 |
| 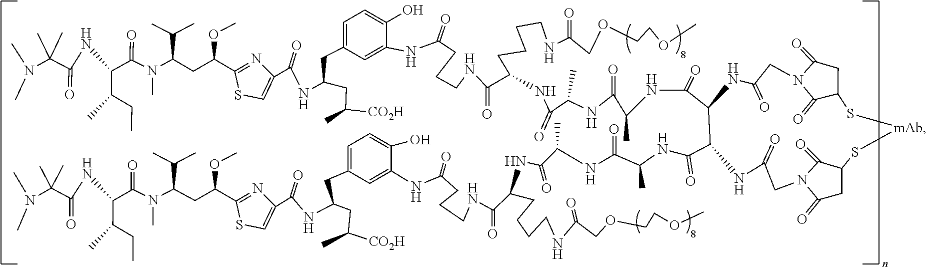 | C-381 |
| 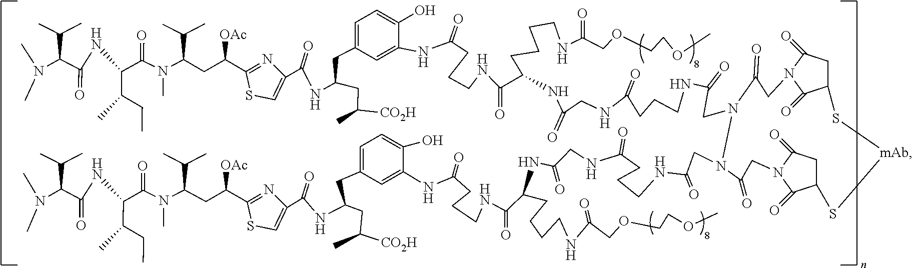 | C-384 |
| 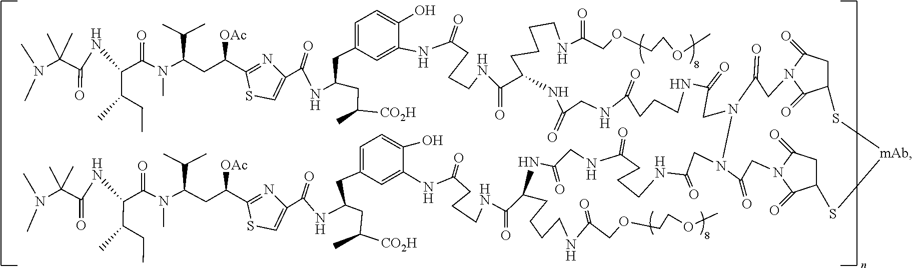 | C-385 |
| 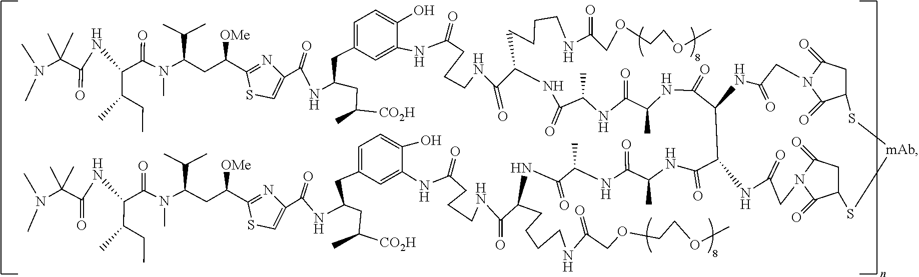 | C-386 |
| 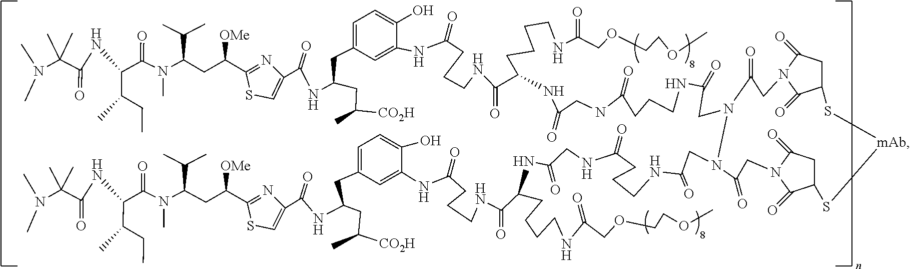 | C-387 |
| 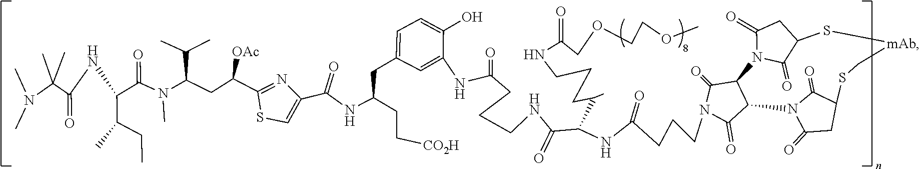 | C-397 |
| 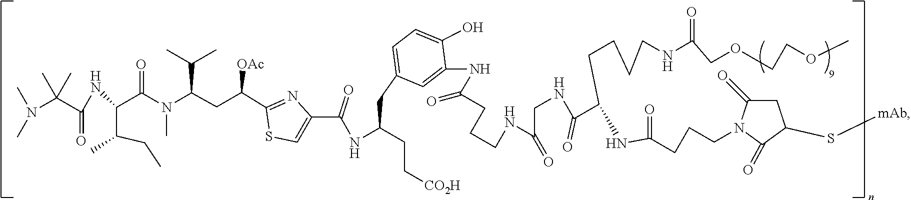 | C-400 |
| 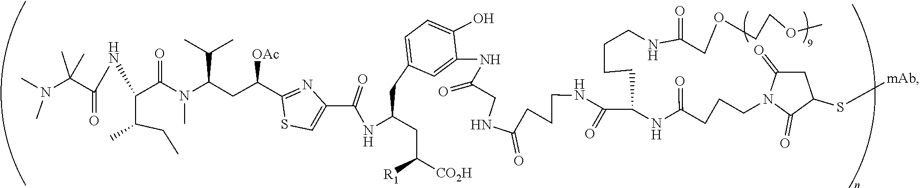 | |
| C-403a, or C-403b, | |
| R₁ = H,C-403a, | |
| R₁ = Me, C-403b | |
| 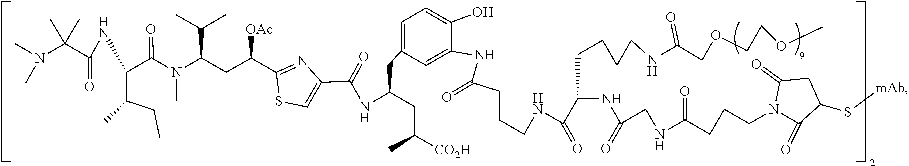 | C-406 |
| 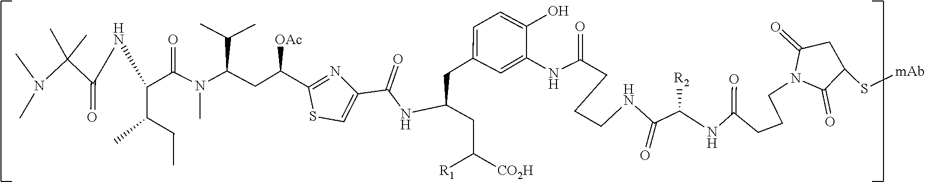 | |
| C-408a: R₁ = (R/S)-Me, R₂ = (CH₂)₄NHCOCH₂O(CH₂CH₂O)₉Me | |
| C-408b: R₁ = (S)-Me, R₂ = (CH₂)₄NHCOCH₂O(CH₂CH₂O)₉Me | |
| C-408c: R₁ = (S)-Me, R₂ = (CH₂)₂CONH(CH₂CH₂O)₈Me | |
| C-408d: R₁ = H, R₂= (CH₂)₄NHCOCH₂O(CH₂CH₂O)₈Me | |
| C-408e: R₁ = H, R₂ = (CH₂)₄NHCOCH₂O(CH₂CH₂O)₈Me | |
| C-408f: R₁ = H, R₂ = (CH₂)₂CONH(CH₂CH₂O)₈Me, | |
| 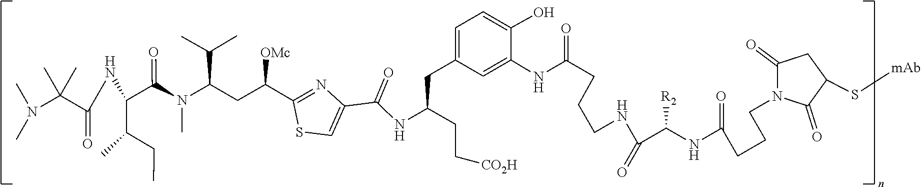 | |
| C-410a: R₂ = (CH₂)₄NHCOCH₂O(CH₂CH₂O)₈Me | |
| C-410b: R₂ = (CH₂)₄NHCOCH₂O(CH₂CH₂O)₉Me | |
| C-410c: R₂ = (CH₂)₂CONH(CH₂CH₂O)₈Me, | |
| 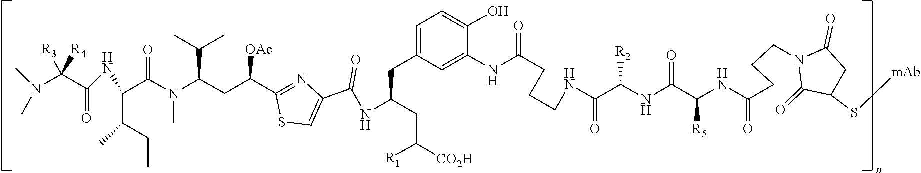 | |
| C-412a: R₃ = H, R₄ = (CH₃)₂CH, R₁ = H, R₂ = (CH₂)₂CONH(CH₂CH₂O)₈Me, R₅ = H; | |
| C-412b: R₃ = R₄ = CH₃, R₁ = H, R₂ = R₅ = (CH₂)₄NHCOCH₂O(CH₂CH₂O)₉Me; | |
| C-412c: R₃ = R₄ = CH₃, R₁ = (S)-Me, R₂ = R₅ = (CH₂)₄NHCOCH₂O(CH₂CH₂O)₈Me; | |
| C-412d: R₃ = H, R₄ = (CH₃)₂CH, R₁ = H, R₅ = (CH₂)₄NHCOCH₂O(CH₂CH₂O)₈Me, R₂ = H; | |
| C-412e: R₃ = R₄ = CH₃, R₁ = (S)-Me, R₂ = (CH₂)₄NHCOCH₂O(CH₂CH₂O)₈Me, R₅ = H; | |
| C-412f: R₃ = R₄ = CH₃, R₁ = (S)-Me, R₅ = (CH₂)₄NHCOCH₂O(CH₂CH₂O)₈Me, R₂ = H; | |
| 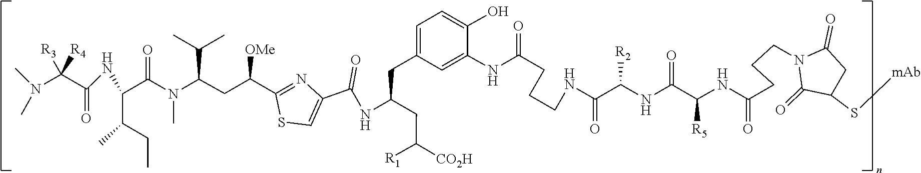 | |
| C-413a: R₃ = H, R₄ = (CH₃)₂CH, R₁ = H, R₂ = (CH₂)₂CONH(CH₂CH₂O)₈Me, R₅ = H; | |
| C-413b: R₃ = R₄ = CH₃, R₁ = H, R₂ = R₅ = (CH₂)₄NHCOCH, O(CH₂CH₂O)₉Me; | |
| C-413c: R₃ = R₄ = CH₃, R₁ = (S)-Me, R₂ = R₅ = (CH₂)₄NHCOCH₂O(CH₂CH₂O)₈Me; | |
| C-413d: R₃ = H, R₄ = (CH₃)₂CH, R₁ = H, R₅ = (CH₂)₄NHCOCH₂O(CH₂CH₂O)₈Me, R₂ = H; | |
| C-413e: R₃ = R₄ = CH₃, R₁ = (S)-Me, R₂ = (CH₂)₄NHCOCH₂O(CH₂CH₂O)₈Me, R₅ = H; | |
| C-413f: R₃ = R₄ = CH₃, R₁ = (S)-Me, R₅ = (CH₂)₄NHCOCH₂O(CH₂CH₂O)₈Me, R₂ = H;, | |
| 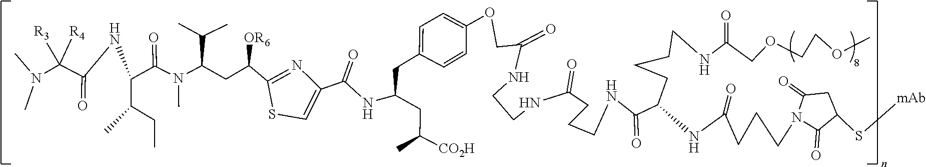 | |
| C-416a: R₃ = R₄ = CH₃, R₆ = Ac; | |
| C-416b: R₃ = R₄ = CH₃, R₆ = Me; | |
| C-416c: R₃ = H, R₄ = (CH₃)₂CH, R₆ = Ac; | |
| C-416d: R₃ = R₄ = CH₃, R₆ = Me;, | |
| 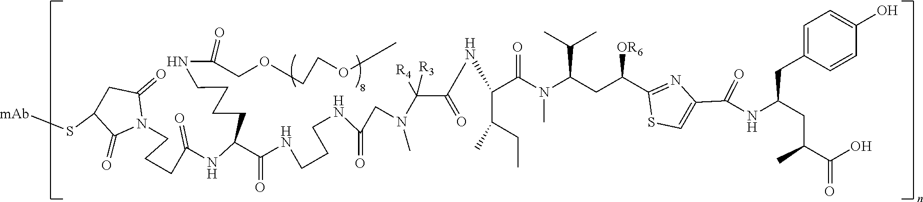 | |
| C-422a: R₃ = R₄ = CH₃, R₆ = Ac; | |
| C-422b: R₃ = R₄ = CH₃, R₆ = Me; | |
| C-422c: R₃ = H, R₄ = (CH₃)₂CH, R₆ = Ac; | |
| C-422d: R₃ = H, R₄ = (CH₃)₂CH, R₆ = Me, | |
| 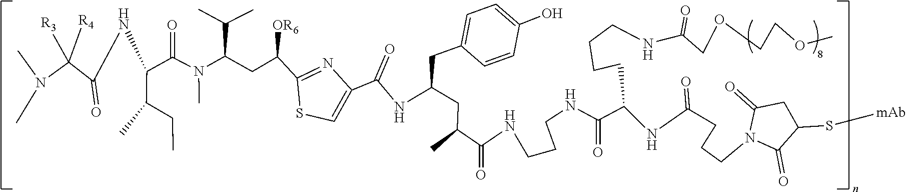 | |
| C-425a: R₃ = R₄ = CH₃, R₆ = Ac; | |
| C-425b: R₃ = R₄ = CH₃, R₆ = Me; | |
| C-425c: R₃ = H, R₄ = (CH₃)₂CH, R₆ = Ac; | |
| C-425d: R₃ = H, R₄ = (CH₃)₂CH, R₆ = Me, | |
| 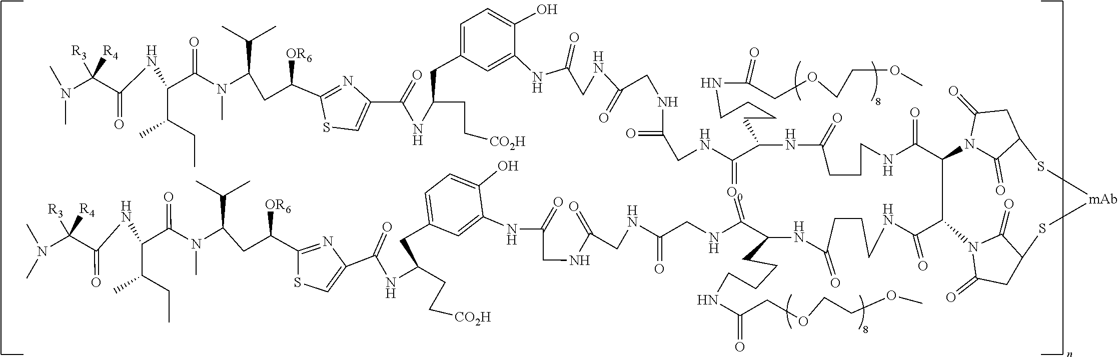 | |
| C-428a: R₃ = R₄ = CH₃, R₆ = Ac; | |
| C-428b: R₃ = R₄ = CH₃, R₆ = Me; | |
| C-428c: R₃ = H, R₄ = (CH₃)₂CH, R₆ = Ac; | |
| C-428d: R₃ = H, R₄ = (CH₃)₂CH, R₆ = Me, | |
| 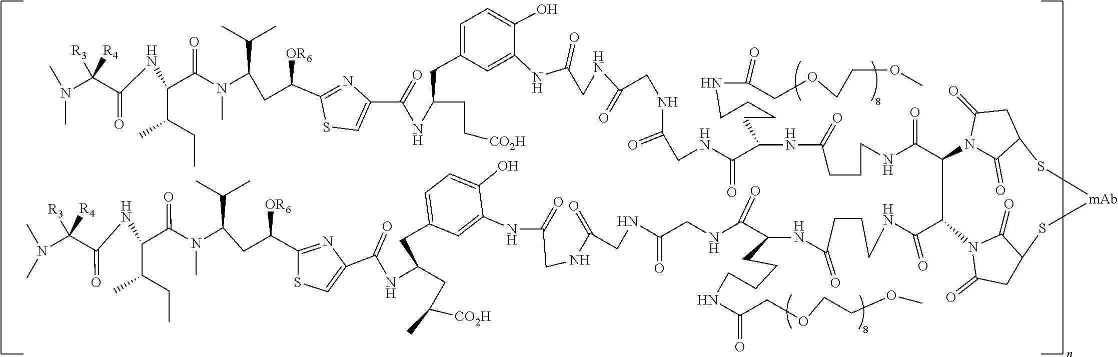 | |
| C-431a: R₃ = R₄ = CH₃, R₆ = Ac; | |
| C-431b: R₃ = R₄ = CH₃, R₆ = Me; | |
| C-431c: R₃ = H, R₄ = (CH₃)₂CH, R₆ = Ac; | |
| C-431d: R₃ = H, R₄ = (CH₃)₂CH, R₆ = Me, | |

TABLE 4
MTT assays of the BCMA antibody conjugates against tumor cells
of NCI-H929, MM1S, 8226-2A1 at 15000 cells, 96 h incubation:

| ADC | DAR | IC50 (nM) | IC50 (nM) | IC50 (nM) |
|---|---|---|---|---|
| Compound | ratio | NCI-H929 | MM1S | 8226-2A1 |
| C-009 | 4.6 | 21.6 | 17.8 | 18.7 |
| C-020 | 4.5 | 18.7 | 19.1 | 17.5 |
| C-025 | 4.6 | 0.68 | 0.92 | 0.46 |
| C-027 | 4.6 | 1.82 | 1.58 | 1.47 |
| C-031 | 4.6 | 1.65 | 1.63 | 1.18 |
| C-037 | 4.6 | 0.48 | 0.85 | 0.63 |
| C-038 | 4.6 | 0.56 | 0.48 | 0.65 |
| C-039 | 4.6 | 1.59 | 1.35 | 1.75 |
| C-043 | 4.5 | 1.22 | 1.48 | 1.89 |
| C-046 | 4.4 | 1.13 | 1.62 | 1.38 |
| C-052 | 7.6 | 2.58 | 3.31 | 3.22 |
| C-056 | 7.8 | 10.3 | 8.2 | 9.75 |
| C-059 | 7.6 | 0.02 | 0.20 | 0.08 |
| C-063 | 7.6 | 0.09 | 0.08 | 0.06 |
| C-066 | 7.8 | 0.02 | 0.07 | 0.04 |
| C-071 | 7.6 | 0.08 | 0.05 | 0.04 |
| C-079 | 4.0 | 2.89 | 4.04 | 5.22 |
| C-084 | 4.0 | 3.48 | 4.85 | 5.73 |
| C-087 | 4.2 | 3.86 | 5.23 | 5.13 |
| C-093 | 7.7 | 0.03 | 0.04 | 0.02 |
| C-096 | 3.6 | 0.38 | 0.81 | 0.72 |
| C-102 | 4.6 | 0.47 | 0.28 | 0.19 |
| C-109 | 4.4 | 0.55 | 0.37 | 0.31 |
| C-111 | 4.5 | 0.17 | 0.23 | 0.36 |
| C-118 | 4.6 | 0.38 | 0.33 | 0.19 |
| C-123 | 4.6 | 0.30 | 0.27 | 0.25 |
| C-133 | 3.6 | 0.39 | 0.29 | 0.18 |
| C-143 | 3.8 | 0.28 | 0.36 | 0.25 |
| C-155 | 3.8 | 0.22 | 0.28 | 0.21 |
| C-168 | 2.2 | 1.25 | 1.43 | 1.09 |
| C-172 | 2.4 | 1.15 | 1.20 | 1.12 |
| C-182 | 4.2 | 0.51 | 0.50 | 0.38 |
| C-186 | 2.8 | 1.68 | 1.11 | 1.28 |
| C-198 | 4.8 | 1.07 | 1.13 | 1.07 |
| C-203 | 3.6 | 1.12 | 1.18 | 1.29 |
| C-208 | 4.6 | 0.28 | 0.27 | 0.22 |
| C-214 | 4.6 | 0.30 | 0.46 | 0.30 |
| C-215 | 4.5 | 0.22 | 0.25 | 0.28 |
| C-216 | 4.6 | 0.48 | 0.32 | 0.38 |
| C-217 | 4.4 | 0.31 | 0.33 | 0.24 |
| C-218 | 4.5 | 0.19 | 0.28 | 0.15 |
| C-226 | 4.4 | 0.06 | 0.05 | 0.03 |
| C-227 | 4.4 | 0.18 | 0.10 | 0.07 |
| C-231 | 4.5 | 0.14 | 0.13 | 0.08 |
| C-237 | 4.2 | 0.51 | 0.80 | 1.08 |
| C-249 | 4.1 | 0.82 | 1.10 | 1.56 |
| C-259 | 4.0 | 0.02 | 0.08 | 0.03 |
| C-260 | 4.1 | 0.05 | 0.09 | 0.03 |
| C-261 | 4.2 | 0.01 | 0.06 | 0.03 |
| C-325 | 4.4 | 0.19 | 0.15 | 0.09 |
| C-326 | 4.4 | 0.10 | 0.17 | 0.12 |
| C-327 | 4.5 | 0.24 | 0.19 | 0.17 |
| C-328 | 4.4 | 0.15 | 0.16 | 0.13 |
| C-329 | 4.5 | 0.13 | 0.15 | 0.11 |
| C-330 | 4.4 | 0.22 | 0.20 | 0.18 |
| C-331 | 4.4 | 0.25 | 0.34 | 0.39 |
| C-332 | 4.5 | 0.25 | 0.29 | 0.23 |
| C-333 | 4.5 | 0.36 | 0.37 | 0.29 |
| C-334 | 4.5 | 0.22 | 0.23 | 0.19 |
| C-335 | 4.5 | 0.40 | 0.39 | 0.37 |
| C-353 | 2.1 | 1.20 | 1.73 | 0.92 |
| C-363 | 1.9 | 1.03 | 1.42 | 0.93 |
| C-371 | 4.1 | 0.62 | 0.66 | 0.91 |
| C-378 | 4.0 | 0.47 | 0.42 | 0.77 |
| C-379 | 4.1 | 0.51 | 0.53 | 0.65 |
| C-380 | 4.0 | 0.53 | 0.50 | 0.57 |
| C-381 | 4.2 | 0.63 | 0.54 | 0.67 |
| C-384 | 4.1 | 0.40 | 0.52 | 0.48 |
| C-385 | 4.1 | 0.46 | 0.62 | 0.66 |
| C-386 | 4.2 | 0.30 | 0.46 | 0.39 |
| C-387 | 4.0 | 0.48 | 0.62 | 0.57 |
| C-397 | 2.2 | 1.10 | 1.39 | 1.27 |
| C-400 | 4.4 | 0.62 | 0.67 | 0.69 |
| C-403a | 4.5 | 0.60 | 0.69 | 0.65 |
| C-403b | 4.4 | 0.19 | 0.22 | 0.29 |
| C-406 | 4.5 | 0.37 | 0.51 | 0.43 |
| C-408a | 4.5 | 0.51 | 0.53 | 0.67 |
| C-408b | 4.4 | 0.41 | 0.43 | 0.49 |
| C-408d | 4.5 | 0.59 | 0.77 | 0.82 |
| C-408e | 4.5 | 0.62 | 0.77 | 0.80 |
| C-408f | 4.4 | 0.65 | 0.76 | 0.85 |
| C-410a | 4.5 | 0.64 | 0.78 | 0.81 |
| C-410b | 4.5 | 0.63 | 0.78 | 0.80 |
| C-410c | 4.6 | 0.65 | 0.79 | 0.81 |
| C-412a | 4.3 | 0.51 | 0.70 | 0.60 |
| C-412b | 4.4 | 0.69 | 0.82 | 0.82 |
| C-412c | 4.5 | 0.61 | 0.77 | 0.65 |
| C-413a | 4.5 | 0.48 | 0.49 | 0.51 |
| C-413b | 4.5 | 0.52 | 0.72 | 0.77 |
| C-413c | 4.5 | 0.39 | 0.52 | 0.51 |
| C-413d | 4.4 | 0.31 | 0.46 | 0.51 |
| C-416a | 4.5 | 0.30 | 0.31 | 0.32 |
| C-416b | 4.4 | 0.30 | 0.45 | 0.53 |
| C-416c | 4.4 | 0.28 | 0.35 | 0.48 |
| C-416d | 4.5 | 0.38 | 0.51 | 0.61 |
| C-422a | 4.6 | 0.72 | 0.83 | 0.89 |
| C-422b | 4.6 | 0.79 | 0.89 | 1.06 |
| C-422c | 4.6 | 0.62 | 0.77 | 0.83 |
| C-422d | 4.5 | 0.68 | 0.82 | 0.91 |
| C-425a | 4.4 | 0.41 | 0.47 | 0.62 |
| C-425b | 4.5 | 0.43 | 0.53 | 0.59 |
| C-425c | 4.5 | 0.31 | 0.37 | 0.48 |
| C-425d | 4.5 | 0.37 | 0.39 | 0.53 |
| C-428a | 4.4 | 0.52 | 0.69 | 0.77 |
| C-428b | 4.4 | 0.49 | 0.73 | 0.71 |
| C-428c | 4.4 | 0.37 | 0.53 | 0.60 |
| C-428d | 4.5 | 0.42 | 0.55 | 0.69 |
| C-431a | 4.5 | 0.23 | 0.34 | 0.47 |
| C-431b | 4.6 | 0.27 | 0.39 | 0.47 |
| C-431c | 4.5 | 0.20 | 0.33 | 0.35 |
| C-431d | 4.4 | 0.25 | 0.37 | 0.39 |
| Paclitaxel | | 2.89 | 3.28 | 3.71 |

### Example 472. Antitumor Activity in vivo (BALB/c Nude Mice Bearing HCC-827, NCI-N87, NCi-H929, BT-474, SK-OV-3, OE-19, Calu-3, HCT-116, Mz-ChA-1, or UCC, Xenograft Tumors independently)

The in vivo efficacy of EGFR conjugates of C-031, C-038, C-066, C-071, C-093, C-111, C-118, C-208, C-214, and C-216 against human lung adenocarcinoma HCC-827 cell; Trop2 conjugates of C-216, C-218, C-328, C-384, C-408b, C-412c, C-422a, C-425a, and C-431c against human gastric carcinoma N-87 cells; BCMA conjugates of C-227, C-403a, C-403b, C-408b, C-412e, C-412f, C-428c, and C-431a against human multiple myeloma NCI-H929 cells; in xenograft models. Five-week-old female BALB/c Nude mice (6 animals per group) were inoculated subcutaneously in the area under the right shoulder with respective carcinoma cells (5×10⁶ cells/mouse) in 0.1-0.2 mL of serum-free medium. The tumors were grown for 6-8 days to an average size of 150 mm³, or 8-9 days to an average size of 180 mm³. The animals were then randomly divided into different groups (6 animals per group). The first group of mice served as the control group and was treated with the phosphate-buffered saline (PBS) vehicle. The other groups were treated with conjugates at dose of 6 mg/Kg administered intravenously. Three dimensions of the tumor were measured every 3 or 4 days (twice a week) and the tumor volumes were calculated using the formula tumor volume=½(length×width×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

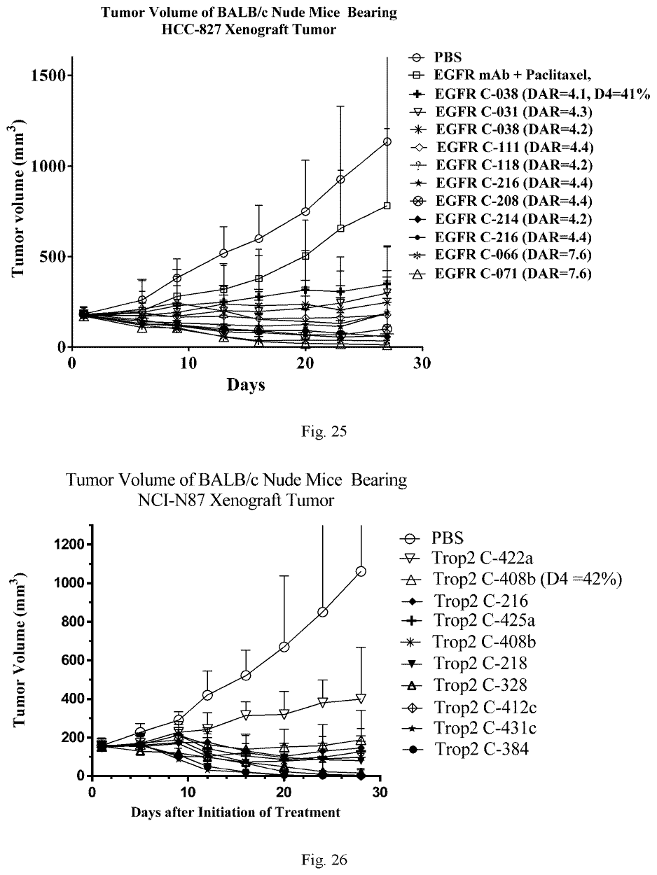

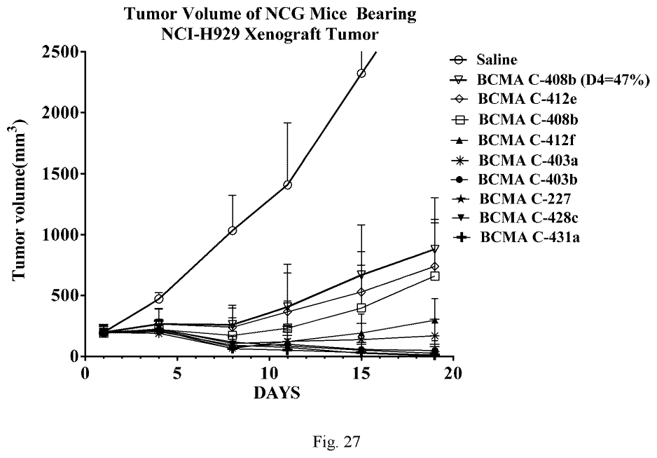

The results were plotted in FIGS. 25-27. All the conjugates did not cause the animal body weight loss at dose of 6.0 mg/Kg. All conjugates demonstrated antitumor activity as comparison with PBS buffer.

### Example 473. Analysis the DAR and the Conjugation Sites by UPLC-MS

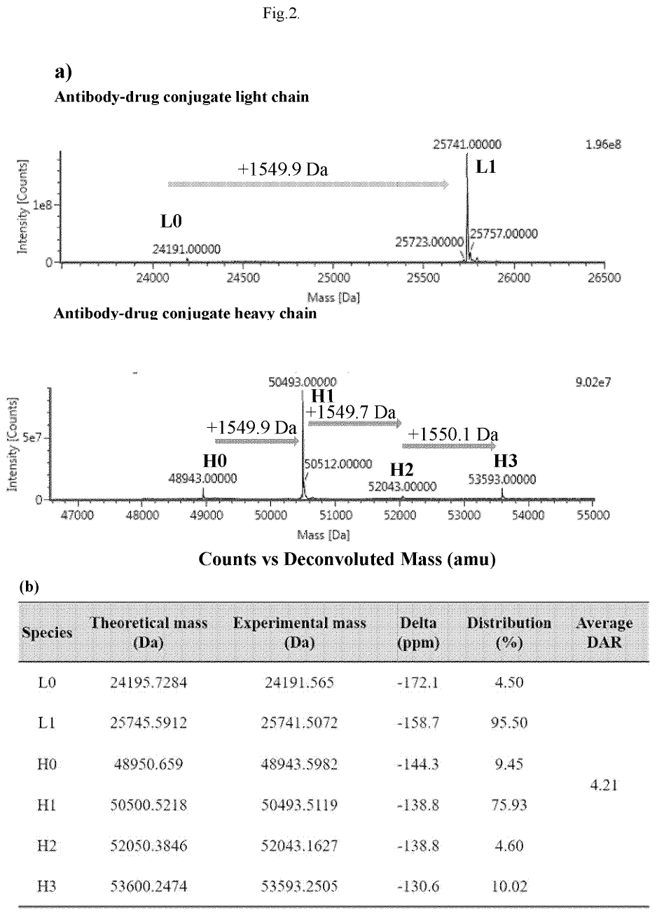

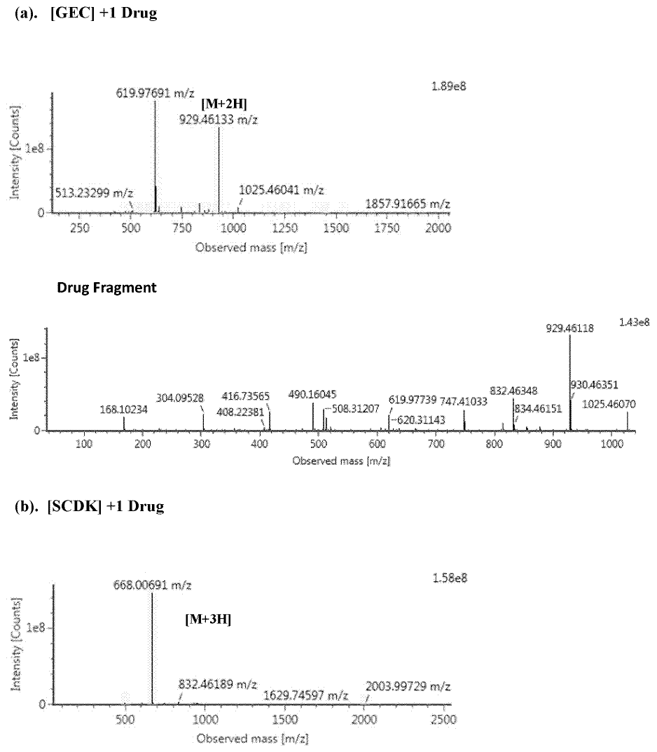

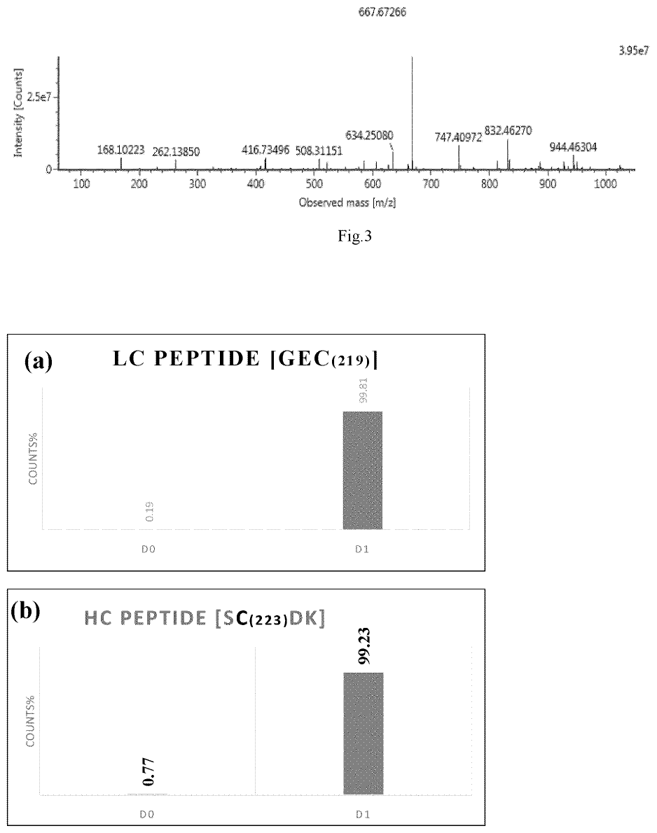

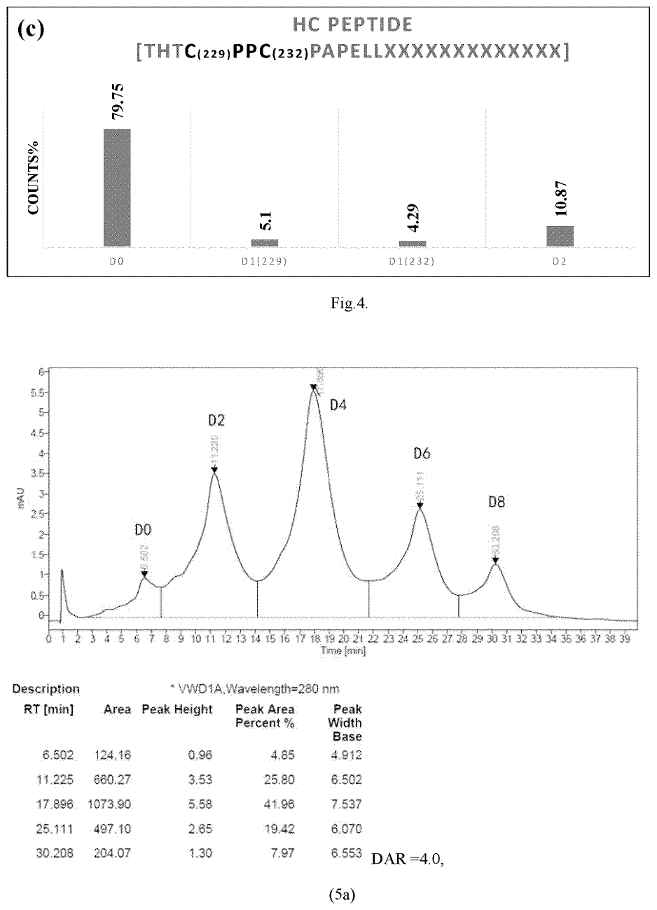

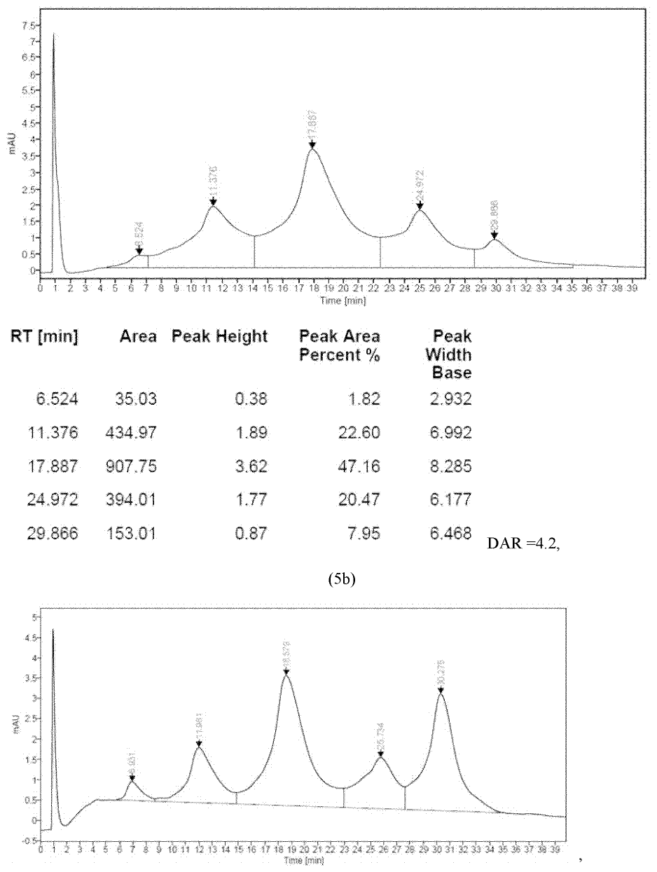

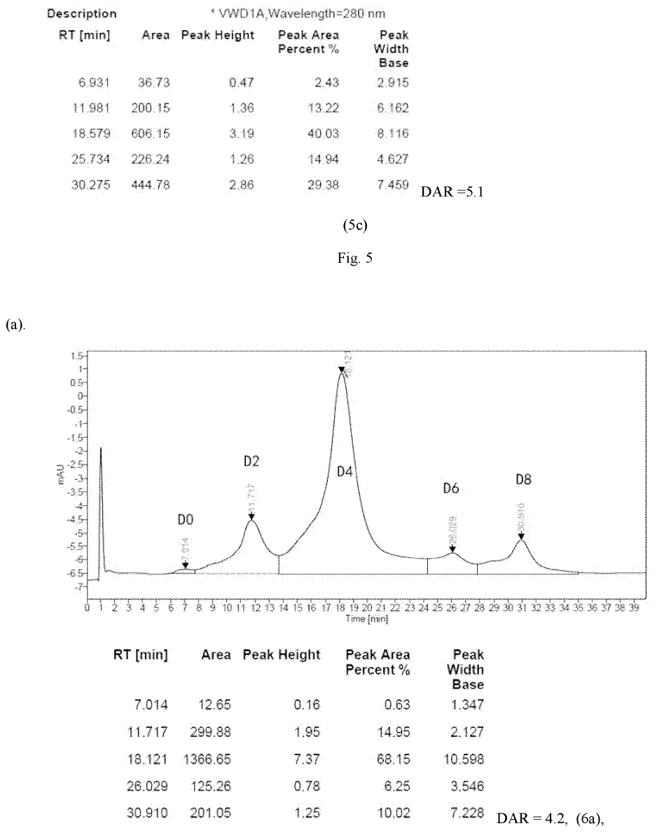

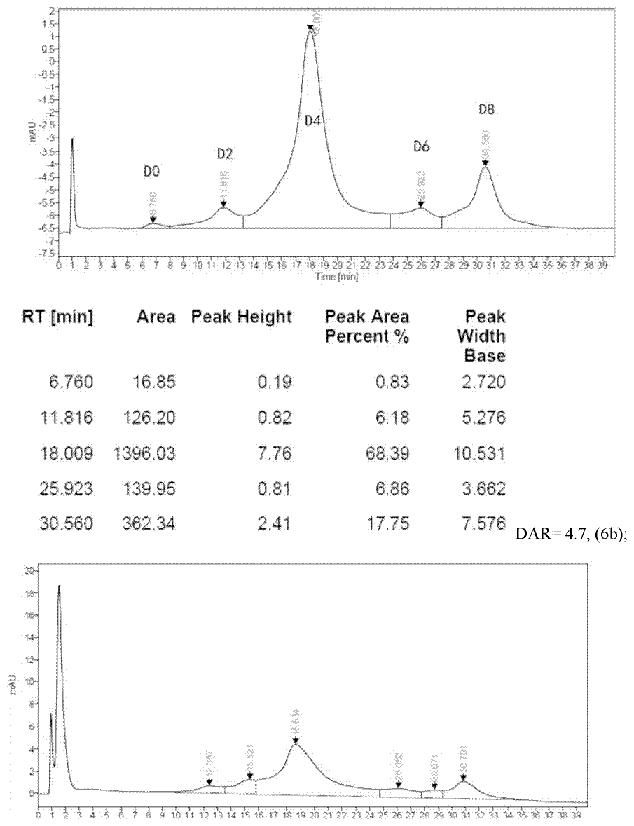

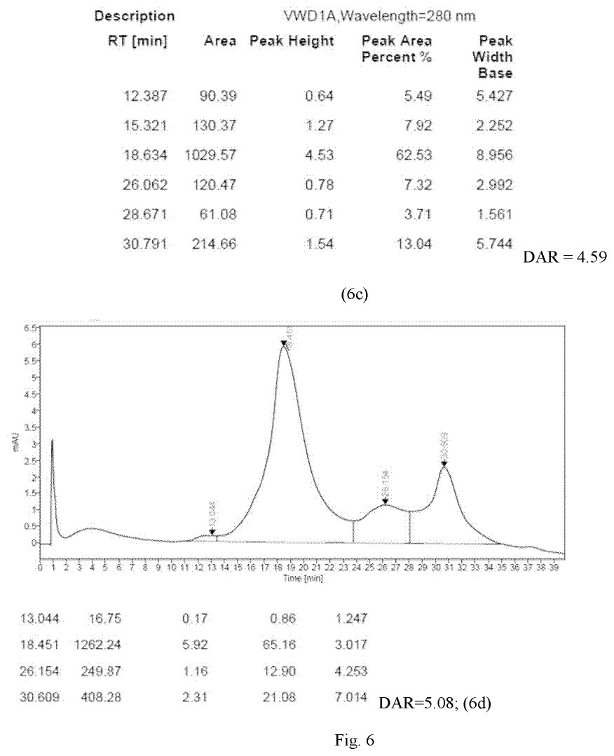

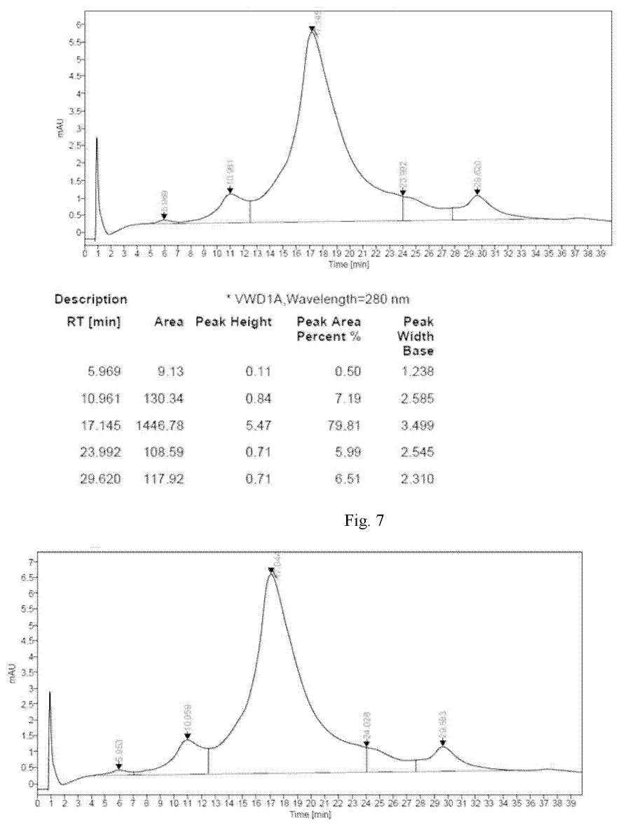

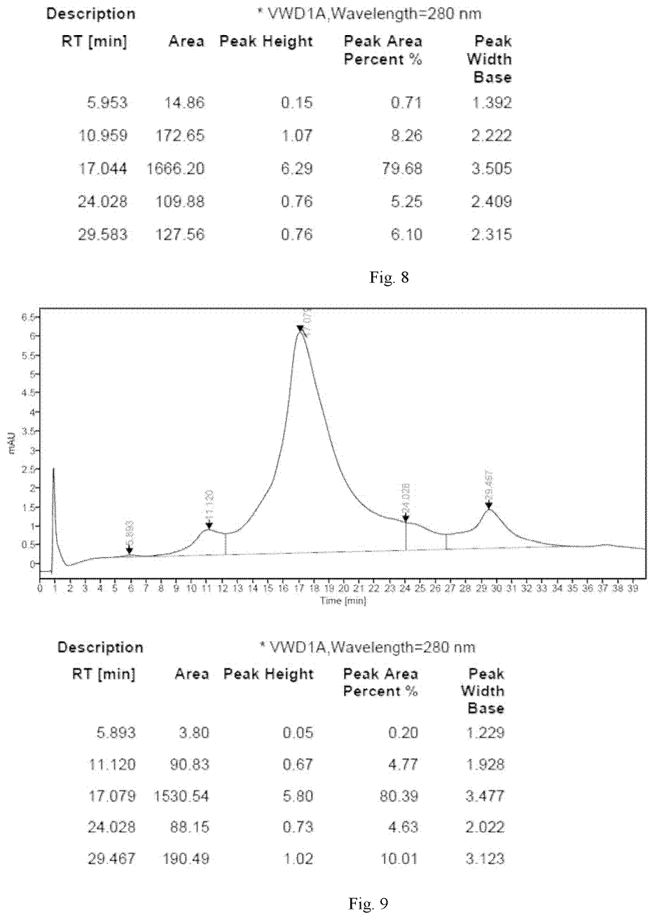

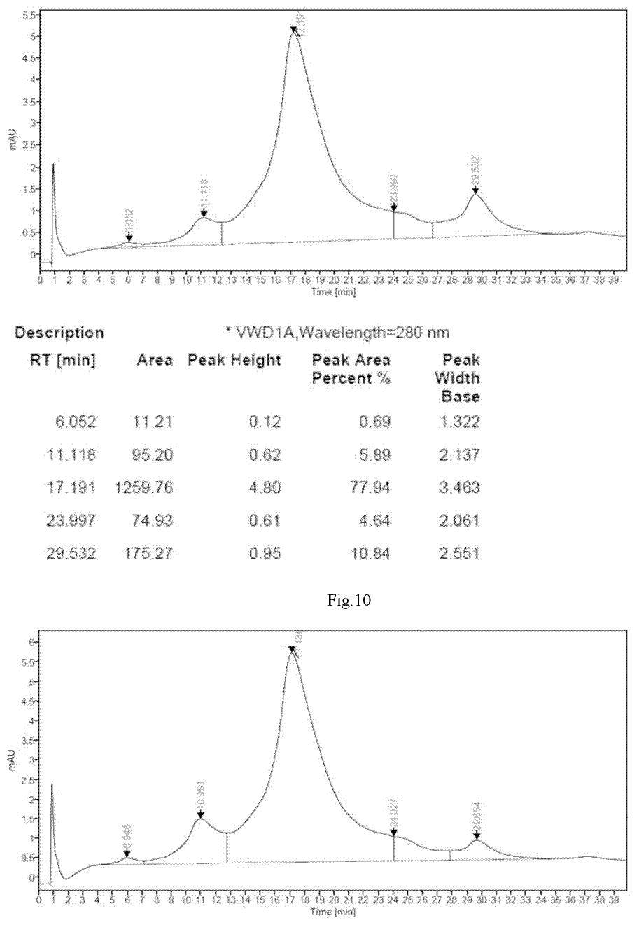

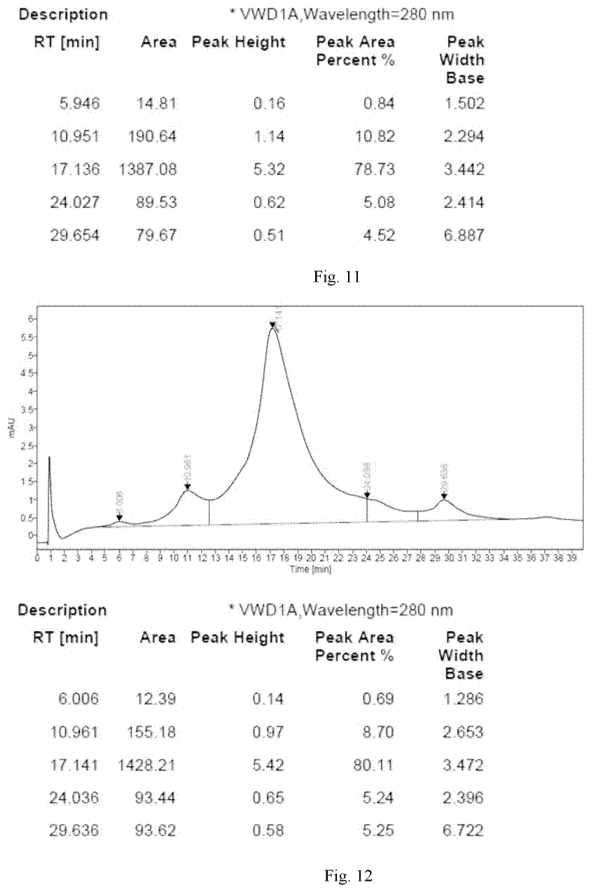

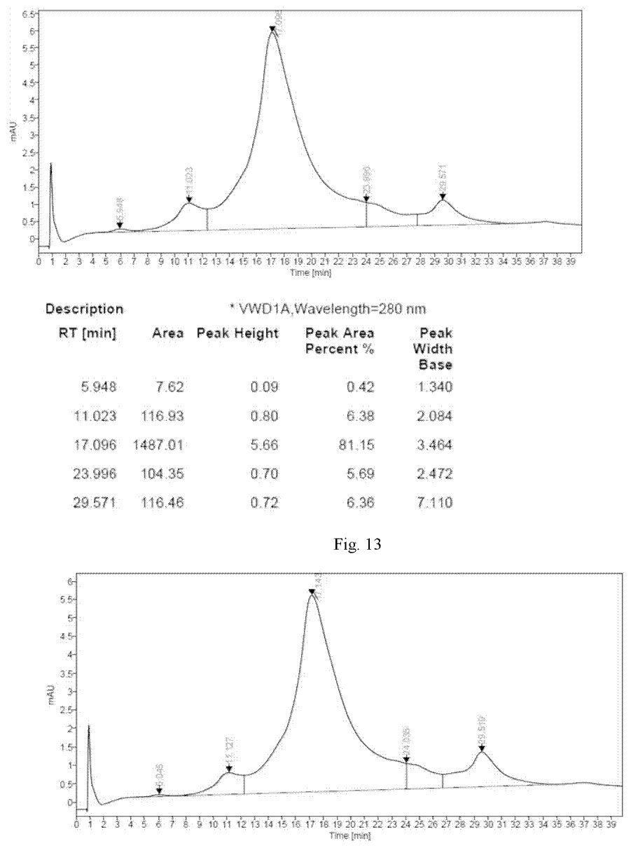

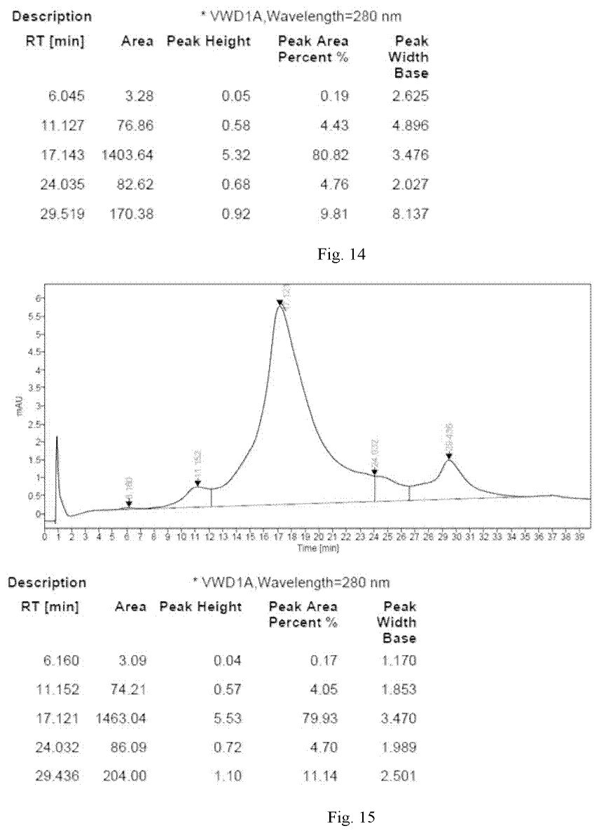

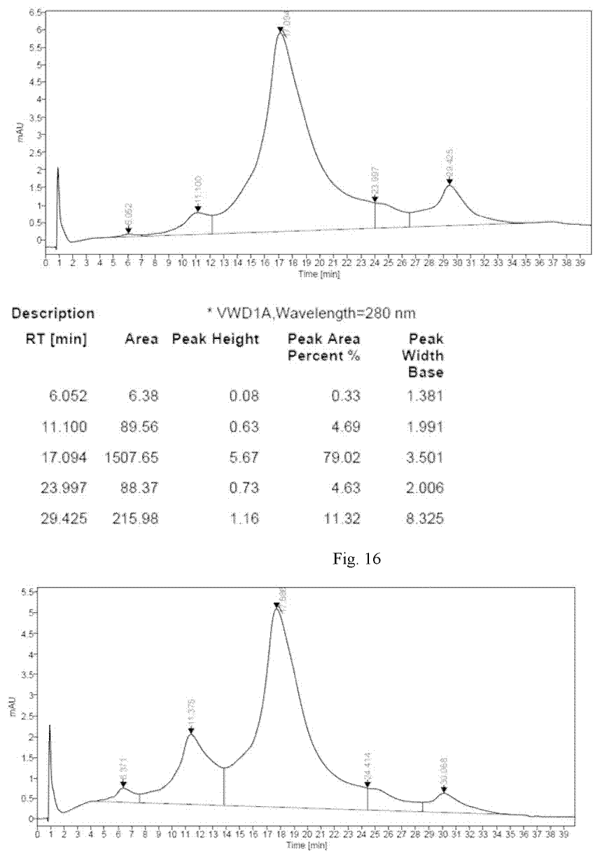

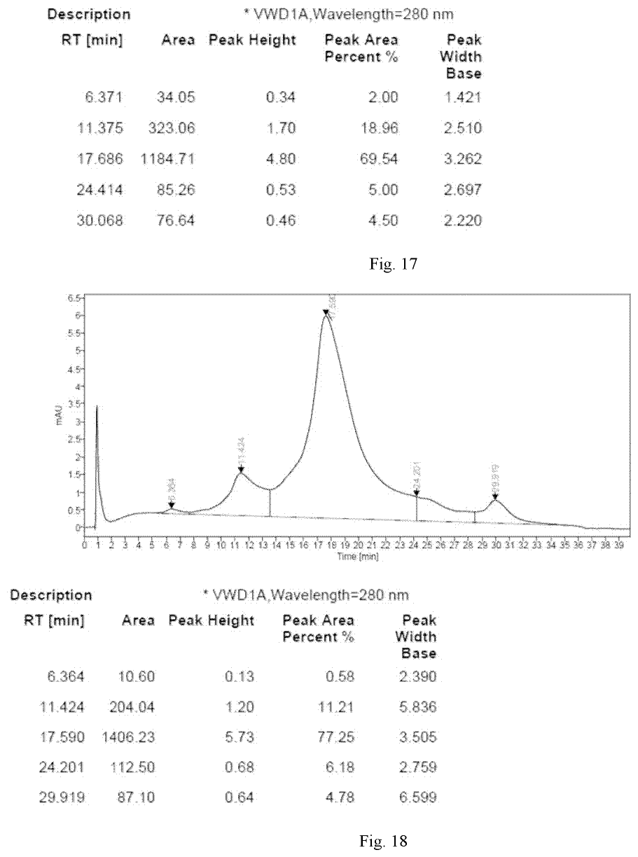

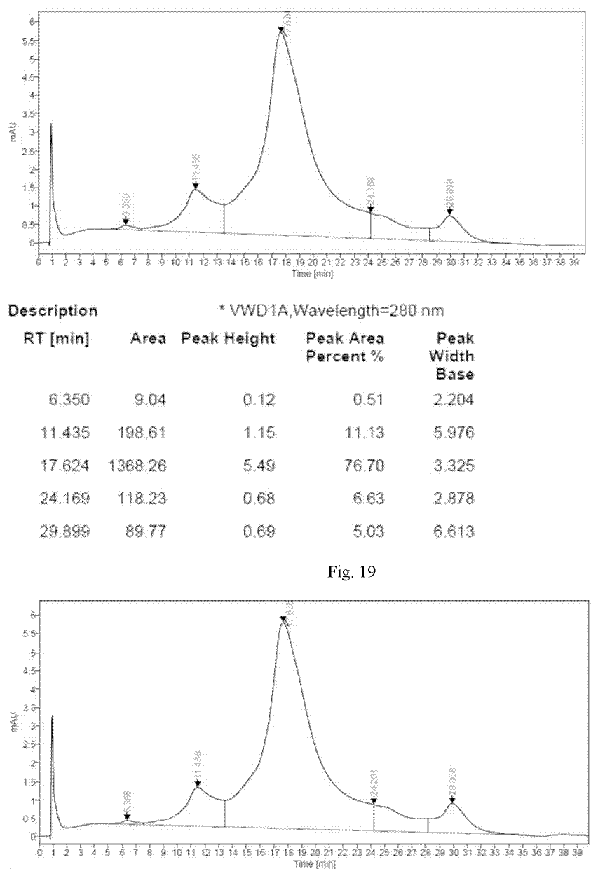

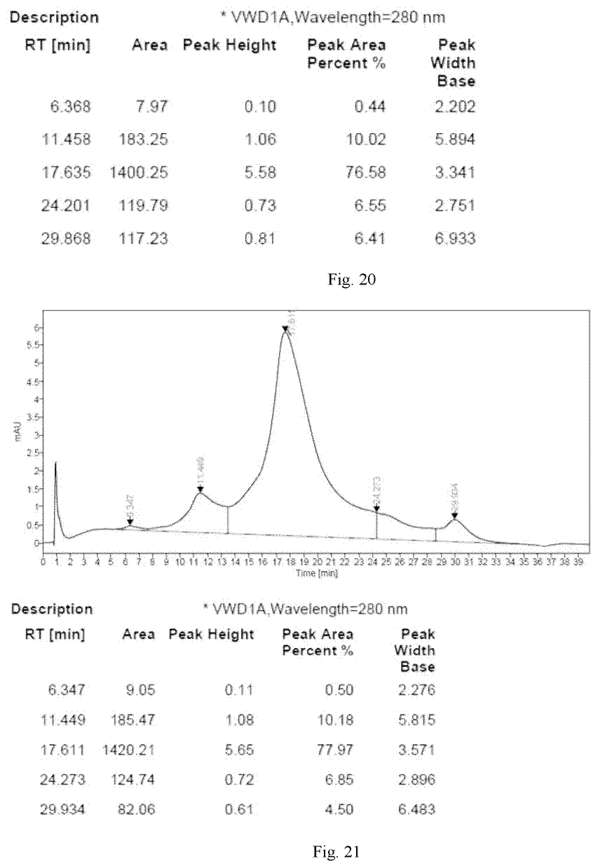

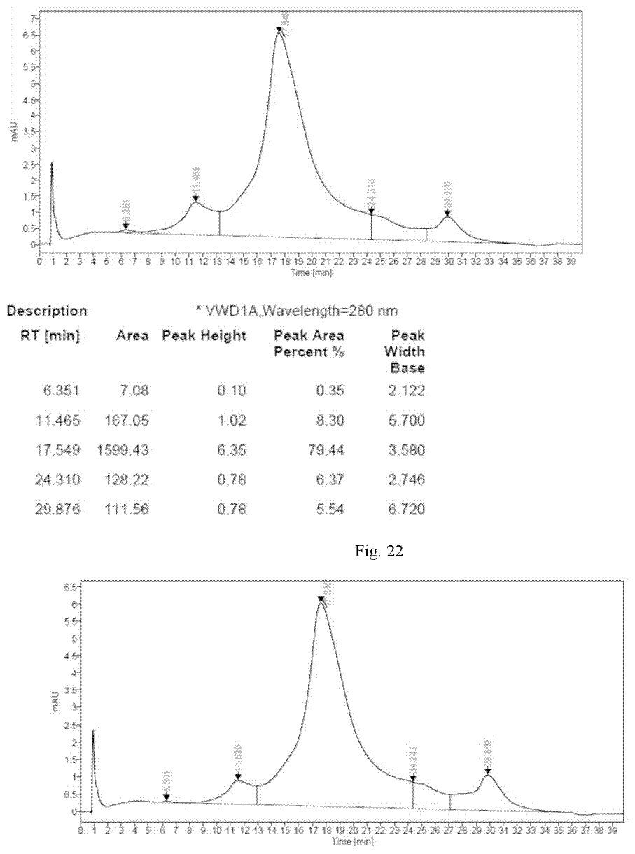

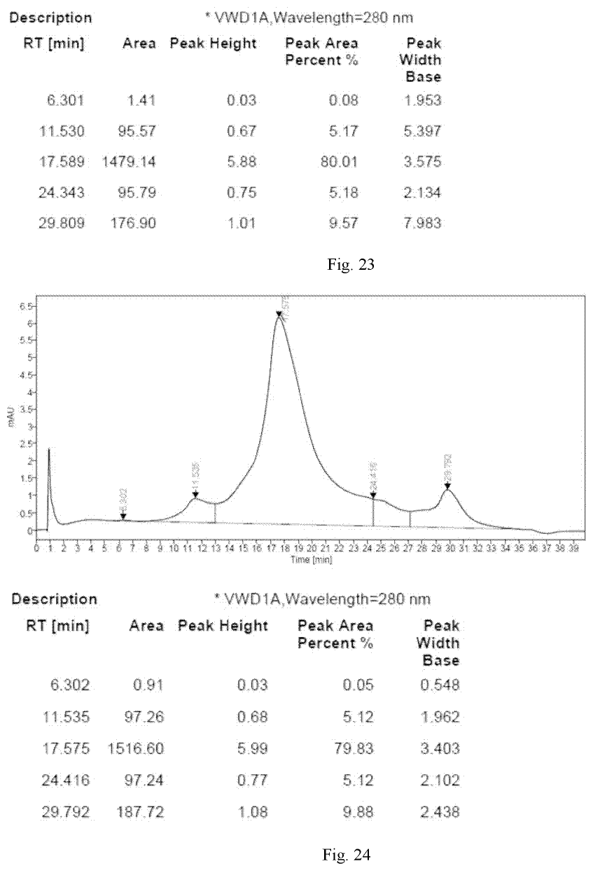

DAR measurement: Reduction (5 mM dithiothreitol at 37° C. for about 2 h) of the ADC followed by a deglycosylation step (Rapid PNGase F at 50° C. for about 15 min) can generate six fragments as illustrated in FIG. 2-4. HC and LC exist as naked or conjugated forms carrying up to 3 payloads. Chromatographic separations of these fragments with MS detection were performed with Acquity UPLC(Waters) using BEH 300 C₄ 1.7 m 2.1×50 mm column coupled with Xevo G2XS Q-TOF mass spectrometer (Waters). Performed the chromatographic separation at a flow rate of 0.25 μl/min using a linear gradient of mobile phase B (ACN with 0.1% FA) from 5% to 85% over 4 min. Conducted the data acquisition with MassLynx software, and used the mass acquisition range from 500 Da to 4000 Da. Performed the data analysis using UNIFI software (Waters). The following equation was used for average DAR calculation for conventional conjugated ADC.

$$Average DAR = L 1 / ( L 0 + L 1 ) * 2 + H 1 / ( H 0 + H 1 + H 2 + H 3 ) * 2 + H 2 / ( H 0 + H 1 + H 2 + H 3 ) * 2 + H 3 / ( H 0 + H 1 + H 2 + H 3 ) * 2$$

Conjugation Site: ADC samples were denatured and reduced (6M Urea, 10 mM dithiothreitol at 56° C. for about 40 min), alkylated (about 30 mM Iodoacetamide, 40 min in the dark at room temperature), diluted in 50 mM NH₄HCO₃ and digested with trypsin (1/50 enzyme/substrate weight ratio, 4 h, 37° C.). Chromatographic separations of peptides with MS detection were performed with Acquity UPLC (Waters) using BEH C₁₈ 1.7 μm 2.1×100 mm Column coupled with Xevo G2XS Q-TOF mass spectrometer (Waters). Perform the chromatographic separation at a flow rate of 0.2 μl/min using a linear gradient of mobile phase B (ACN with 0.1% FA) from 1% to 40% over 95 min. Conducted the data acquisition with MassLynx software, and used the mass acquisition range from 100 Da to 2500 Da. Perform the data analysis using UNIFI software (Waters).

1.-22. (canceled)

23. A homogenous conjugation process comprising the following steps:
(a) incubating an antibody or antibody-like protein in presence of an effective amount of a transition metal cation-amino chelate and a reductant, in a water based buffer system to selectively reduce inter-chain disulfide bonds within the antibody or antibody-like protein to generate thiol groups;
(b) introducing an effective amount of a linker or drug/linker complex bearing thiol reactive groups to react with the thiol groups resulted from step (a);
(c) optionally adding an effective amount of an oxidant to re-oxidize unreacted thiol groups; and
(d) purifying resulted conjugate;
wherein the optional step (c) can be replaced by: adding an effective amount of cystine or a relative disulfide compound to quench unreacted reductant, while generating cysteine or a thiol compound from reduction of the cystine or a disulfide compound to quench excessive conjugation linker or drug/linker complex containing a thiol reactive group, or an effective amount of cysteine or relative thiol compound can be added to quench excessive linker or linker/payload complex molecule;
wherein the transition metal cation-amino chelate/complex has the formula of M(NR₁R₂R₃)ₘ₁^{m2+}, wherein M is Zn²⁺, Cu²⁺, Fe²⁺, Cd²⁺, Ni²⁺, Cr²⁺, Cr³⁺, Ti²⁺, Ti³⁺, Co²⁺, Mn²⁺, Mn³⁺, Ag⁺, or Hg²⁺; wherein R₁, R₂ and R₃ are independently a C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; m1 is 1, 2, 3, 4, 5, 6, 7 or 8; m2 is 1, 2, 3, 4, 5, or 6; or N, R₁, R₂ and/or R₃ themselves can form a heterocyclic, carbocyclic, diheterocyclic, or dicarbocyclic ring; and (NR₁R₂R₃)ₘ₁ can form a dimer, trimer, tetramer, pentamer, or hexamer wherein these polymers are covalently linked by N, R₁, R₂ and/or R₃; the transition metal cation-amino chelate/complex, M (NR₁R₂R₃)ₘ₁^{m2+}, used in step (a) is 0.01 mM-1.0 mM in concentration, or 0.5-20 equivalents in moles of the antibody or antibody-like protein;
wherein the reductant is an organic phosphine reductant and used at 1.0-20 equivalents in moles of the antibody or antibody-like protein;
wherein the oxidant in step (c) is DHAA, Fe³⁺, I₂, Cu²⁺, Mn³⁺, MnO₂, or a mixture of Fe³⁺/I⁻, the oxidant used in a reaction solution of step (c) is 0.02 mM-1.0 mM in concentration, or 1-100 equivalents in moles of the antibody or antibody-like protein;
wherein a pH in a conjugation reaction of step (b) is between about 5.0 to 8.0;
wherein the reaction solution of step (a) comprises up to 30% of a water mixable (miscible) organic solvent selected from DMA, DMF, ethanol, methanol, acetone, acetonitrile, THF, isopropanol, dioxane, propylene glycol, or ethylene diol as a co-solvent in the water based buffer system;
wherein a temperature in the conjugation reaction of step (b) is between about −5° C. to 40° C.; a reaction time of step (b) is between about 15 min to about 48 hours;
wherein the resulted conjugate is purified by gel filtration on a Sephadex G25 or Sephacryl S300 column, adsorption chromatography, ion (cation or anion) exchange chromatography or by dialysis (ultrafiltration or hyperfiltration (UF) and/or diafiltration (DF).

24. The process according to claim 23, wherein the transition metal cation-amino chelate is selected from the group consisting of Zn(NH₂CH₃)₂²⁺, Zn(NH₂CH₂CH₃)₂²⁺, Zn(NH₂CH₂CH₂CH₃)₂²⁺, Zn(NH₂CH(CH₃)₂)₂²⁺, Zn(NH₂C(CH₃)₃)₂²⁺, Zn(NH₂CH₂C(CH₃)₃)₂²⁺, Zn(NH(CH₃)₂)₂²⁺, Zn(NH(CH₂CH₃)₂)₂²⁺, Zn(NH(CH(CH₃)₂)₂)₂²⁺, Zn(NH(C(CH₃)₃)₂)₂²⁺, Zn(NH(CH(CH₂CH₃)₂)₂)₂²⁺, Zn(NH(CH₂C(CH₃)₃)₂)₂²⁺, Zn(NH(CH₂C(CH₂CH₃)₃)₂)₂²⁺, Zn(NH(CH₂CH₂C(CH₃)₃)₂)₂²⁺, Zn(NH₂CH₂CH₂OH)₂²⁺, Zn(NH(CH₂CH₂OH)₂)₂²⁺, Zn(N(CH₂CH₂OH)₃)₂²⁺, Zn(NH₂CH₂COOH)₂²⁺, Zn(NH₂CH₂CONH₂)₂²⁺, Zn(NH₂CH₂COOCH₃)₂²⁺, Zn(NH₂CH₂COOCH₂CH₃)₂²⁺, Zn(NH₂CH₂COOC(CH₃)₃)₂²⁺, Zn(NH₂CH₂COOCH(CH₃)₂)₂²⁺, Zn(NH₂CH₂CH₂COOH)₂²⁺, Zn(NH(CH₂COOH)₂)₂²⁺, Zn(N(CH₂CH₂COOH)₃)₂²⁺, Zn(NH₂CH₃)₄²⁺, Zn(NH₂CH₂CH₃)₄²⁺, Zn(NH₂CH₂CH₂CH₃)₄²⁺, Zn(NH₂CH(CH₃)₂)₄²⁺, Zn(NH₂C(CH₃)₃)₄²⁺, Zn(NH₂CH₂C(CH₃)₃)₄²⁺, Zn(NH(CH₃)₂)₄²⁺, Zn(NH(CH₂CH₃)₂)₄²⁺, Zn(NH(CH(CH₃)₂)₂)₄²⁺, Zn(NH(C(CH₃)₃)₂)₄²⁺, Zn(NH(CH(CH₂CH₃)₂)₂)₄²⁺, Zn(NH(CH₂C(CH₃)₃)₂)₄²⁺, Zn(NH(CH₂C(CH₂CH₃)₃)₂)₄²⁺, Zn(NH(CH₂CH₂C(CH₃)₃)₂)₄²⁺, Zn(NH₂CH₂CH₂OH)₄²⁺, Zn(NH(CH₂CH₂OH)₂)₄²⁺, Zn(N(CH₂CH₂OH)₃)₄²⁺, Zn(NH₂CH₂COOH)₄²⁺, Zn(NH₂CH₂CONH₂)₄²⁺, Zn(NH₂CH₂COOCH₃)₄²⁺, Zn(NH₂CH₂COOCH₂CH₃)₄²⁺, Zn(NH₂CH₂COOC(CH₃)₃)₄²⁺, Zn(NH₂CH₂COOCH(CH₃)₂)₄²⁺, Zn(NH₂CH₂CH₂COOH)₄²⁺, Zn(NH(CH₂COOH)₂)₄²⁺, Zn(N(CH₂CH₂COOH)₃)₄²⁺, 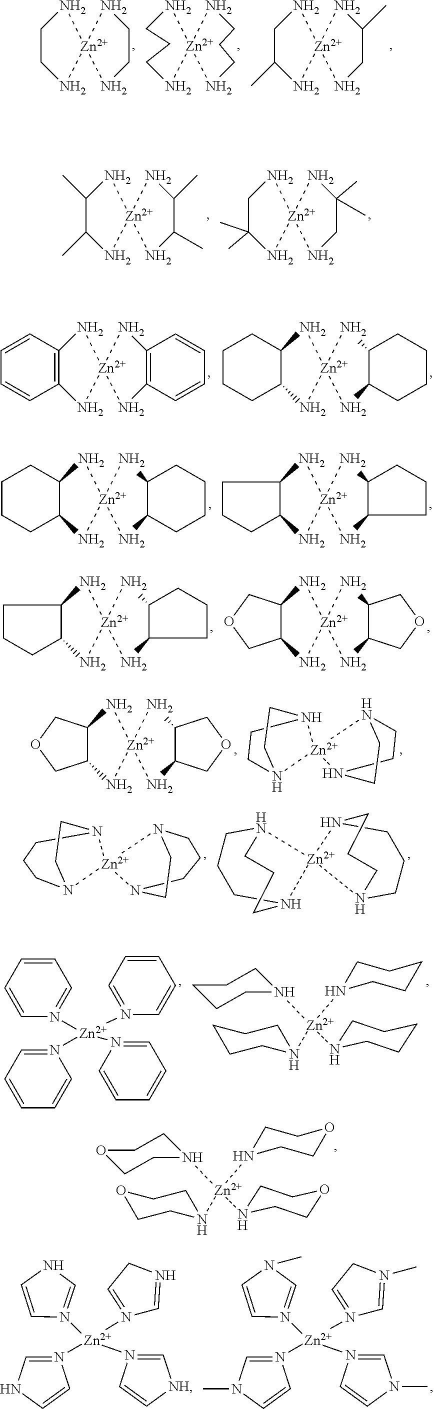 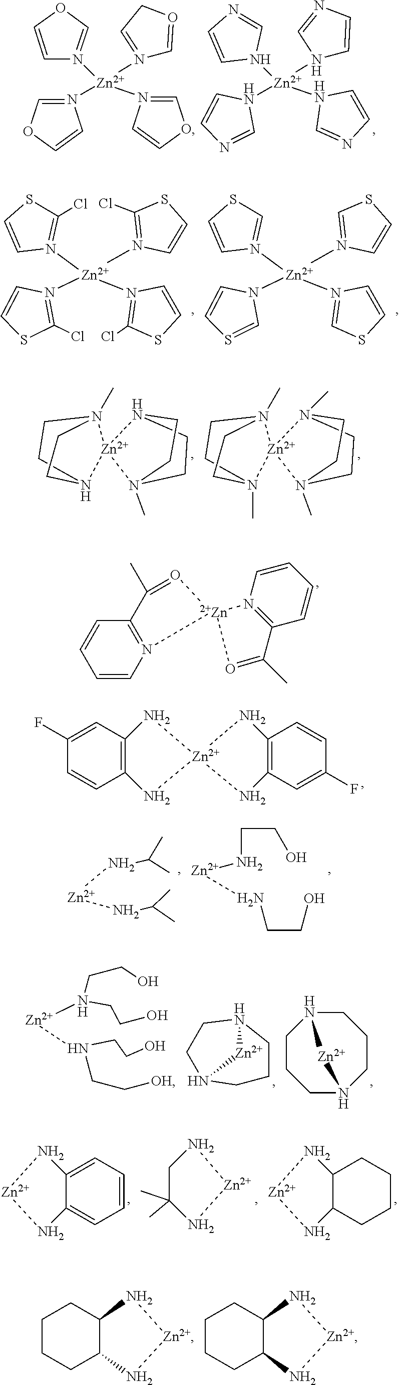 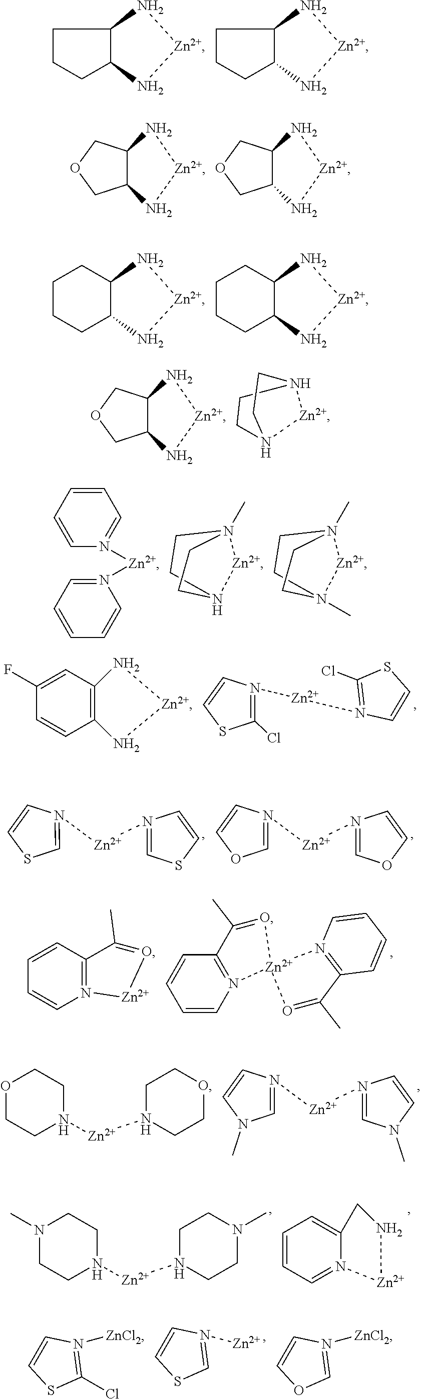 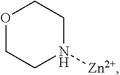
and a salt formed from any of the above cations with an anion which is selected from the group consisting of Cl⁻, Br⁻, I⁻, SO₄²⁻, HSO₄⁻, NO₃⁻, PO₄³⁻, HPO₄²⁻, H₂PO₄⁻, CO₃²⁻, HCO₃⁻, HCOO⁻, CH₃COO⁻, F₃CCOO⁻, Cl₃CCOO⁻, FCH₂COO⁻, ClCH₂COO⁻, F₂CHCOO⁻, Cl₂CHCOO⁻, BF₄⁻, SO₃²⁻, HSO₃⁻, CH₃SO³⁻, C₆H₅CH₂SO³⁻, C₆H₅SO³⁻, C₆H₅COO⁻, C₆H₅CH₂COO⁻, C₆F₅O⁻, C₆H₄(OH)COO⁻, C₆H₂F₃O⁻, C₆H₄(NO₂)O⁻, and C₆H₂(NO₂)₃O⁻;
wherein the transition metal cation-amino complex in the reaction solution of step (a) are 0.5-20 equivalents in moles of the protein, and is added with a water-miscible organic solvent, selected from ethanol, methanol, propanol, propandiol, DMA, DMF, DMSO, THF, or CH₃CN.

25. The process according to claim 23, wherein the organic phosphine reductant is selected from the group consisting of Tris(2-carboxyethyl)phosphine (TCEP) (P(CH₂CH₂COOH)₃), Tris(hydroxypropyl)phosphine (P(CH₂CH₂CH₂OH)₃), P(CH₂CH₃)₃, P(CH₂CH₂CH₃)₃, P(CH₂CH₂—CH₂CH₃)₃, P(CH(CH₃)₂)₃, P(CH₂CH═CH₂)₃, P(CH₂CH₂CN)₃, P(CH(CH₃)₂)₂(CH₂CH₂NH₂), P(CH₂CH₂CONH₂)₃, P(CH₂CH₂CONHCH₃)₃, P(CH₂CH₂CH₂NHCOCH₃)₃, NaB(CN)H₃, (C₆H₁₁)₂P(CH₂)₄P(C₆H₁)₂, (C₆H₁₁)₂P(CH₂)₃P(C₆H₁₁)₂, Dicyclohexyl(ethyl)phosphine, Bis[2-(di-tert-butylphosphino)-ethyl]amine, Tricyclohexyl-phosphine, 1, 2-Ethanediylbis[dicyclohexyl]-phosphine, Bis[2-(dicyclohexylphosphino)-ethyl]amine, Tris[2-(diphenylphosphino)ethyl]-phosphine ([(C₆H₅)₂PCH₂CH₂]₃P), triphenylphosphine, sulfonylated triphenylphosphines (2-(diphenylphosphino)benzenesulfonic acid (diPPBS), 3-(diphenylphosphino)benzenesulfonic acid, 4-(diphenylphosphino)benzenesulfonic acid, and 3, 3′, 3″-phosphinetriyltribenzenesulfonic acid); and the reductant used in the reaction solution of step (A) is 2.0-4.0 equivalents in moles of the protein.

26. The process according to claim 23, wherein the buffer system comprises PBS, Mes, Bis-Tris, Bis-Tris Propane, Pipes, Aces, Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetate, Histidine, Citrate, MES, Borate, or a combination of two, three or four buffer components from above; a pH of the buffer system is 4.0-9.0; a concentration of the buffer in the reaction solution of step (A) is 0.02-1.0 M.

27. The process according to claim 23, wherein the drug/linker complex has a formula (I), (II) or (III): 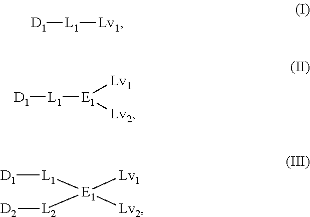
wherein: Lv₁ and Lv₂ are a thiol reaction group, and are independently selected from the group consisting of: 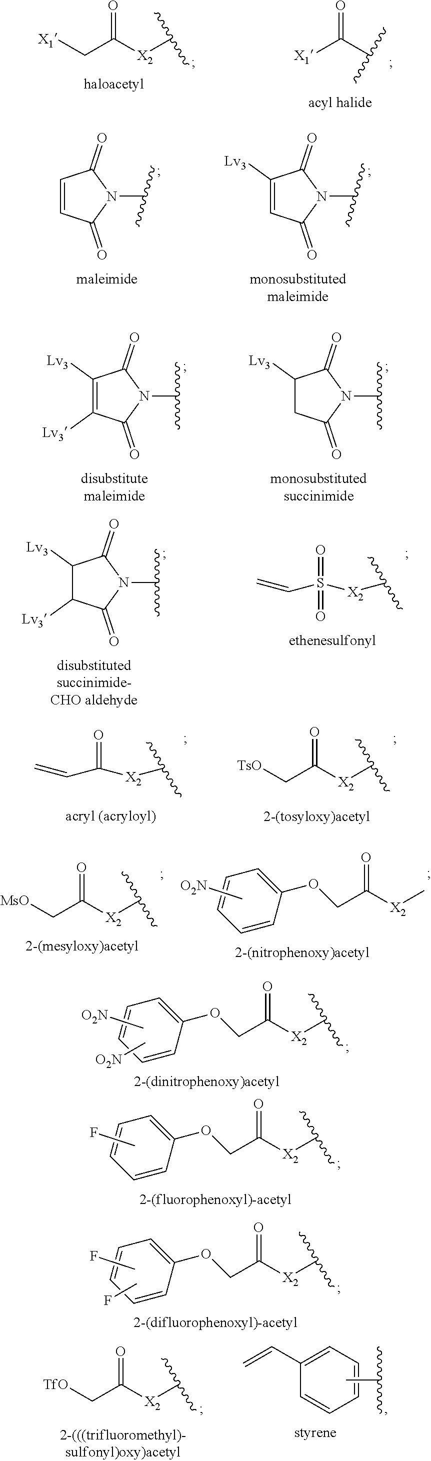 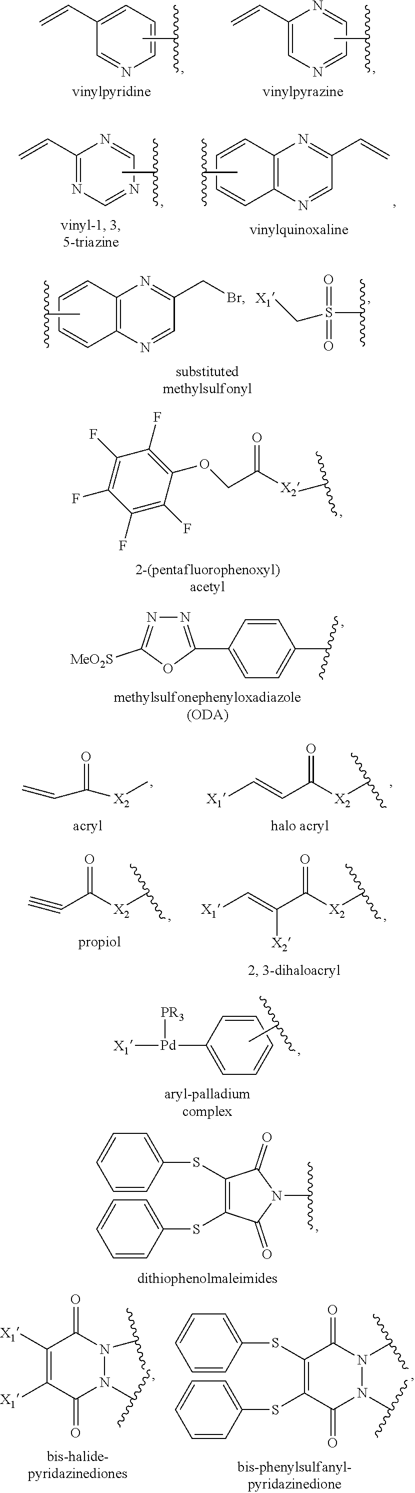 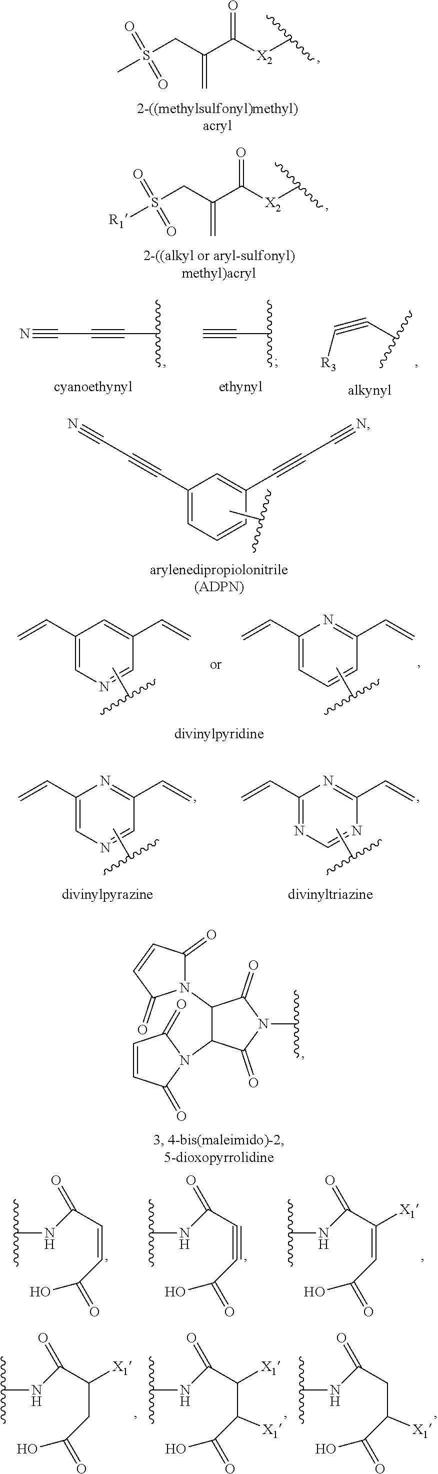 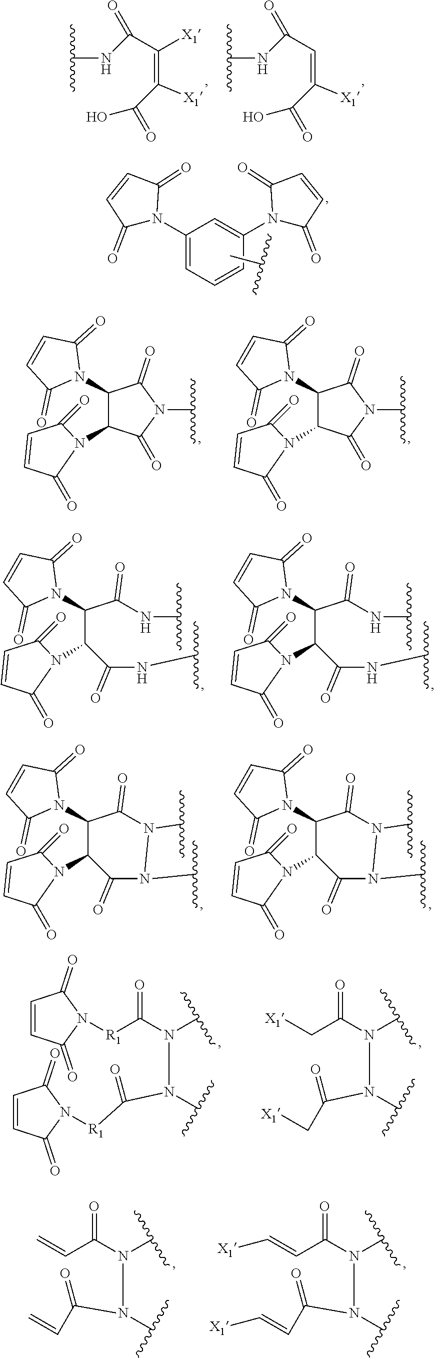 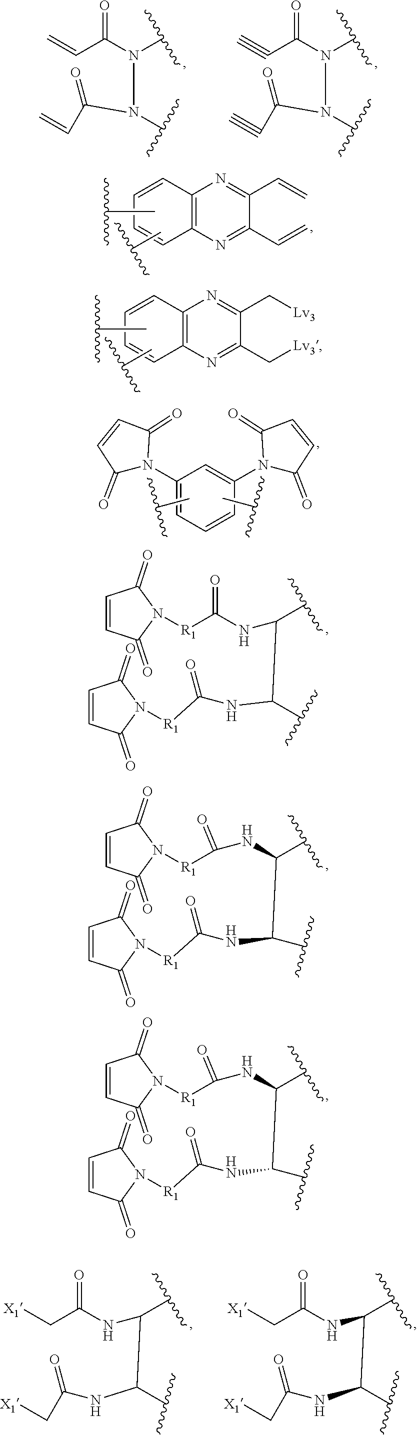 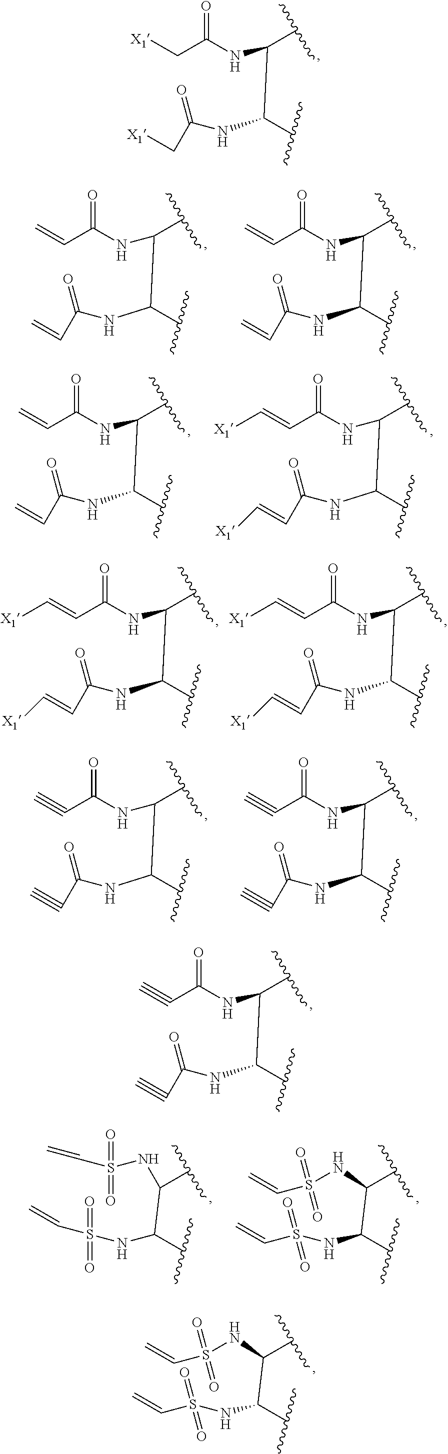 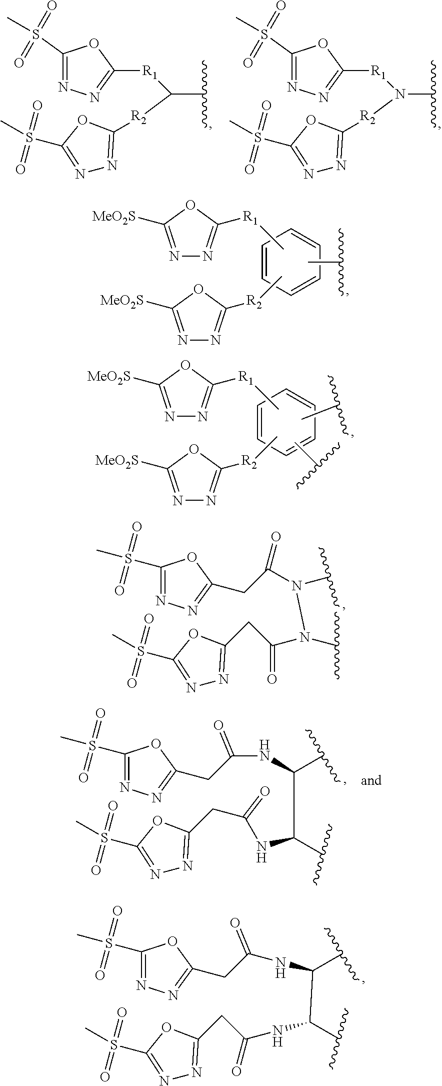
wherein X₁′ and X₂′ are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, an anhydride formed of any of the above itself, or formed with another anhydride:acetyl anhydride, formyl anhydride; or an intermediate molecule generated of any of the above with a condensation reagent for peptide coupling reactions or for Mitsunobu reactions;
in the formula (II) and formula (III), the groups of 2 and 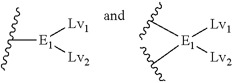
is independently selected from: 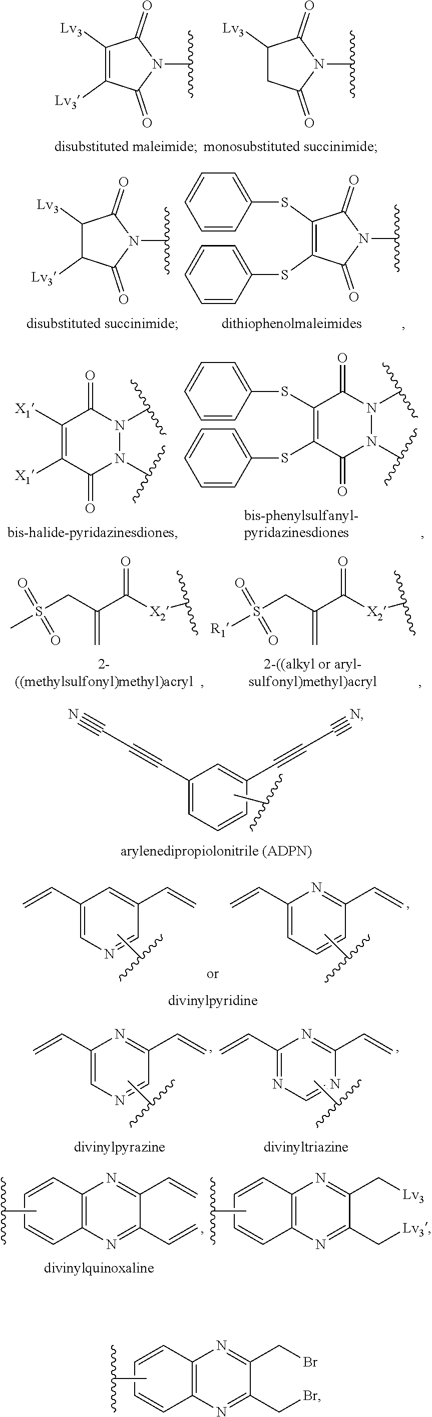 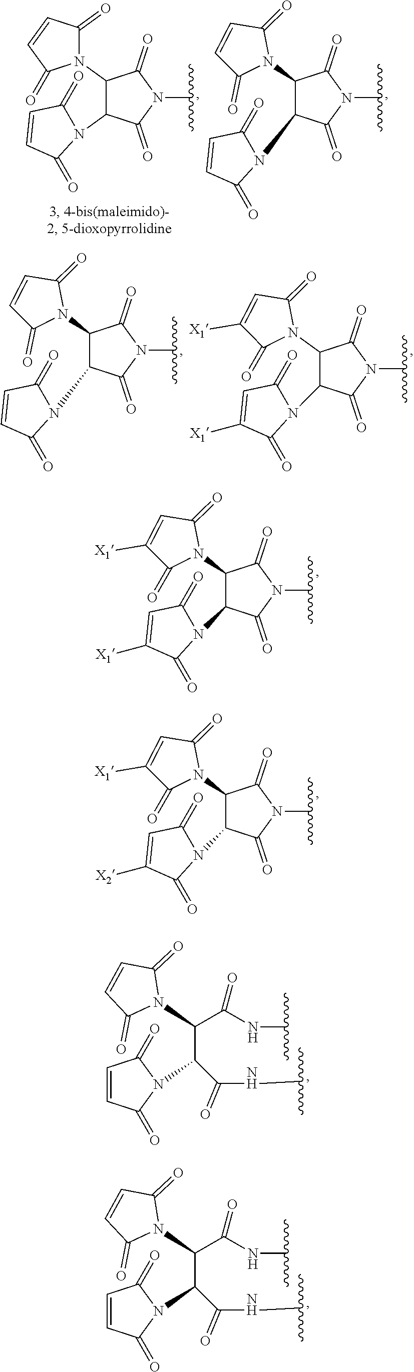 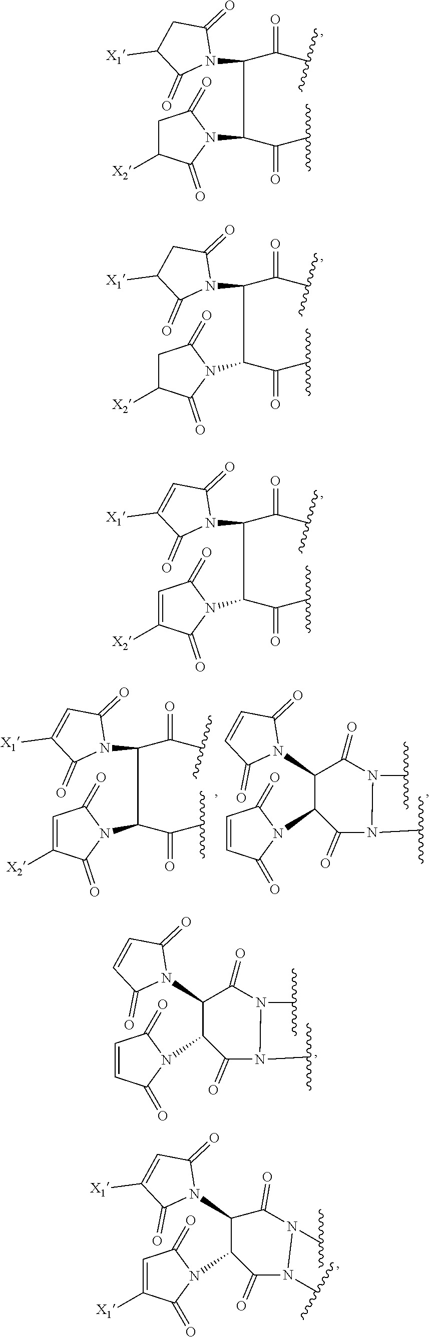 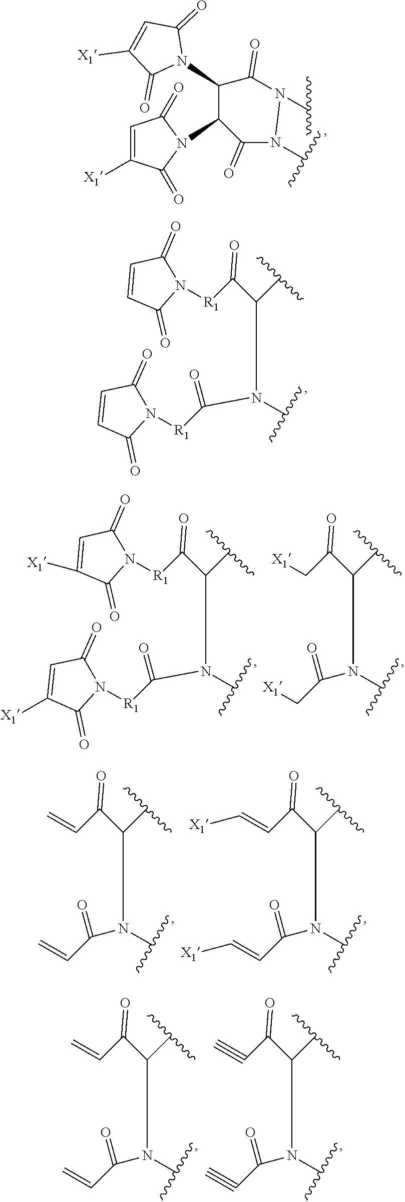 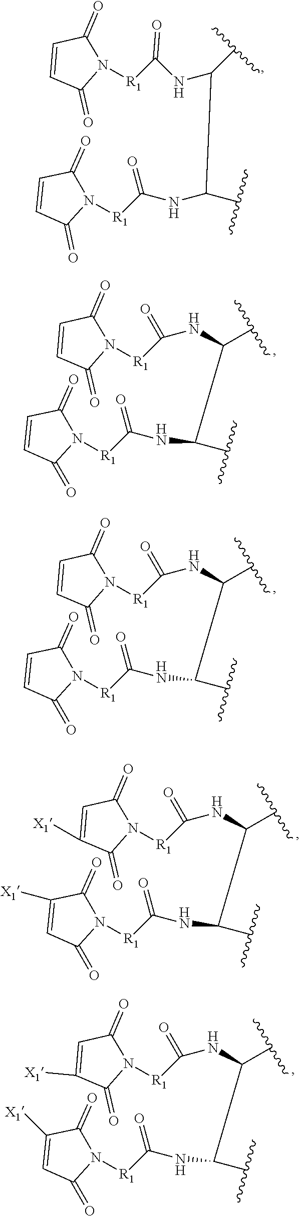 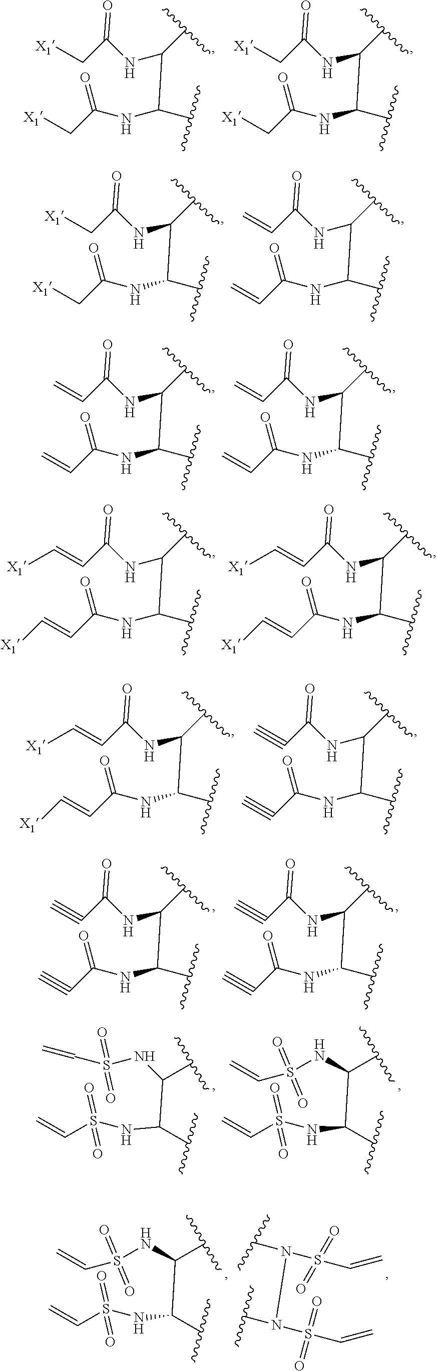 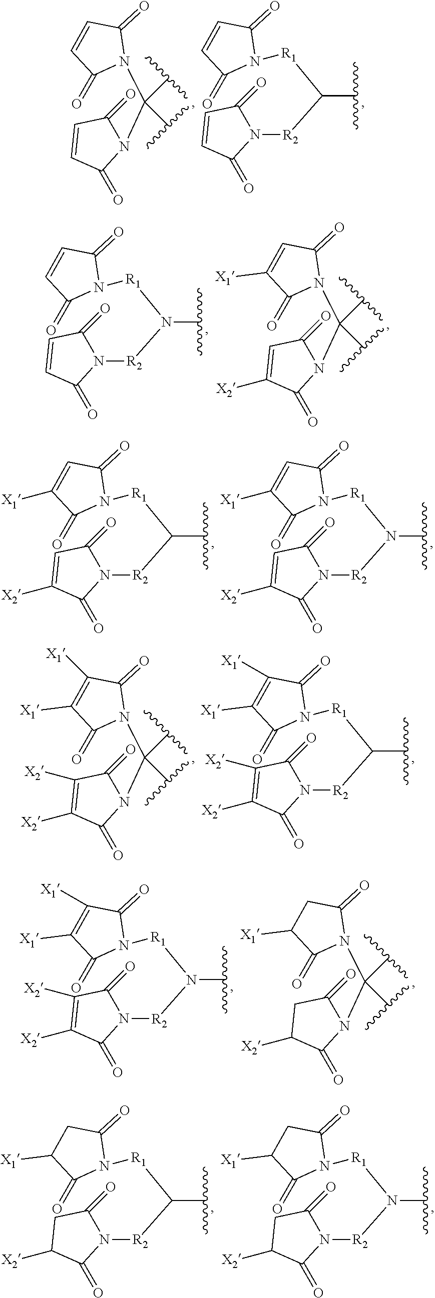 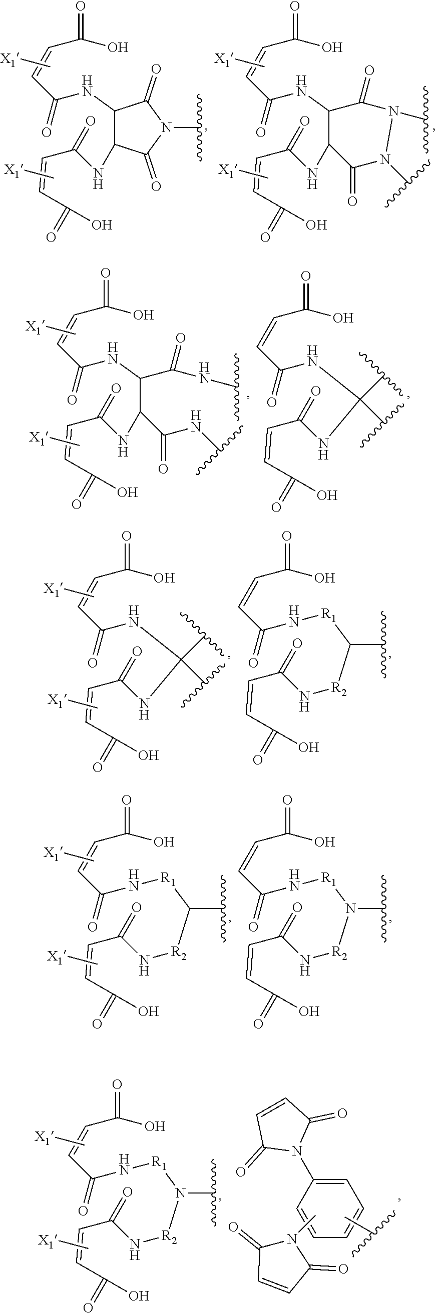 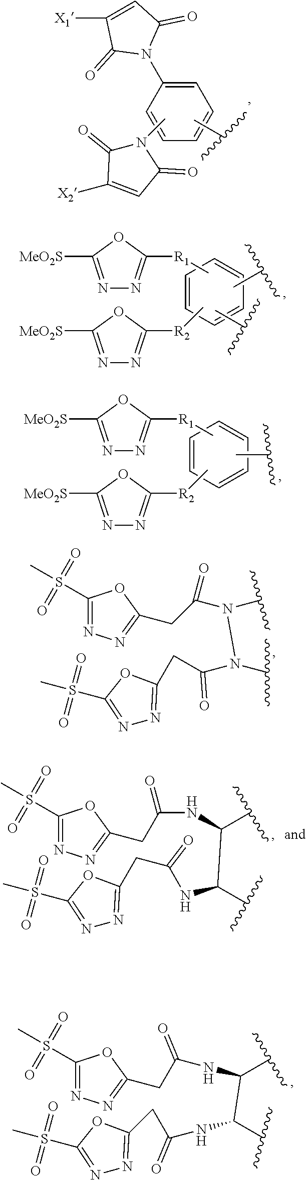
wherein L_{V3}, L_{V3}′, X₁′ and X₂′ are described above; a connecting bond“ [symbol] ” in the middle of two atoms means it can link either of the two atoms;
wherein L₁ and L₂ are, the same or different, independently O, NH, S, NHNH, N(R₃), N(R₃)N(R_{3′}), polyethyleneoxy unit of formula (OCH₂CH₂)ₚOR₃, (OCH₂CH(CH₃))ₚOR₃, NH(CH₂CH₂O)ₚR₃, or NN(CH₂CH(CH₃)O)ₚR₃, N[(CH₂CH₂O)ₚR₃][(CH₂CH₂O)_{p′}R_{3′}], (OCH₂CH₂)ₚCOOR₃, CH₂CH₂(OCH₂CH₂)ₚCOOR₃, wherein p and p′ are an integer independently selected from 0 to about 1000; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; wherein R₃ and R_{3′} are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or 1-8 natural or unnatural amino acids; or polyethyleneoxy unit of formula (OCH₂CH₂)ₚ or (OCH₂CH(CH₃))ₚ, wherein p is an integer from 0 to about 1000, or a combination of two or more of the above;
wherein L₁ or L₂ may contain a self-immolative or a non-self-immolative component, peptidyl unit, a hydrazone bond, a disulfide, an ester, an oxime, an amide, or a thioether bond, the self-immolative unit is an aromatic compound that is electronically similar to a para-aminobenzylcarbamoyl (PAB) group, and is selected from the group consisting of 2-aminoimidazol-5-methanol compounds, heterocyclic PAB compounds, beta-glucuronide, and ortho or para-aminobenzylacetals;
wherein the self-immolative linker component has one of the following structures: 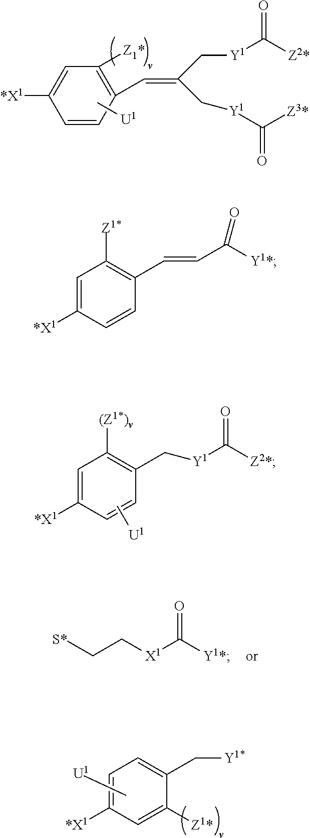
wherein a (*) atom is a point of attachment of additional spacer or releasable linker unit, or a cytotoxic agent, and/or a cell binding molecule (CBA); X¹, Y¹, Z² and Z³ are independently NH, O, or S; Z¹ is independently H, NH, O or S; v is 0 or 1; U¹ is independently H, OH, C₁-C₆ alkyl, (OCH₂CH₂)ₙF, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(ORs(OR₅′) wherein R₅ and R₅′ are as defined above;
wherein the non-self-immolative linker component is one of the following structures: 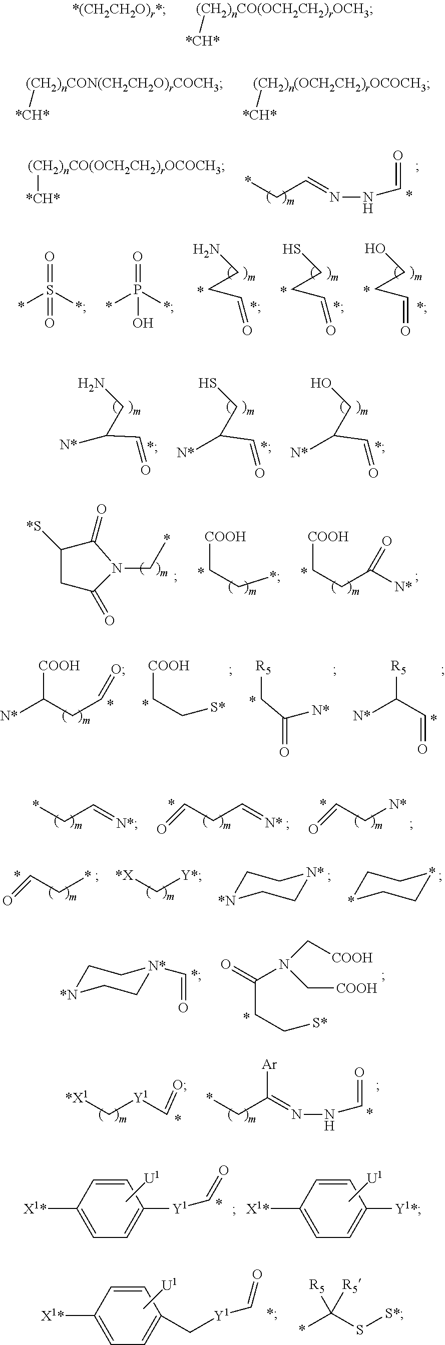 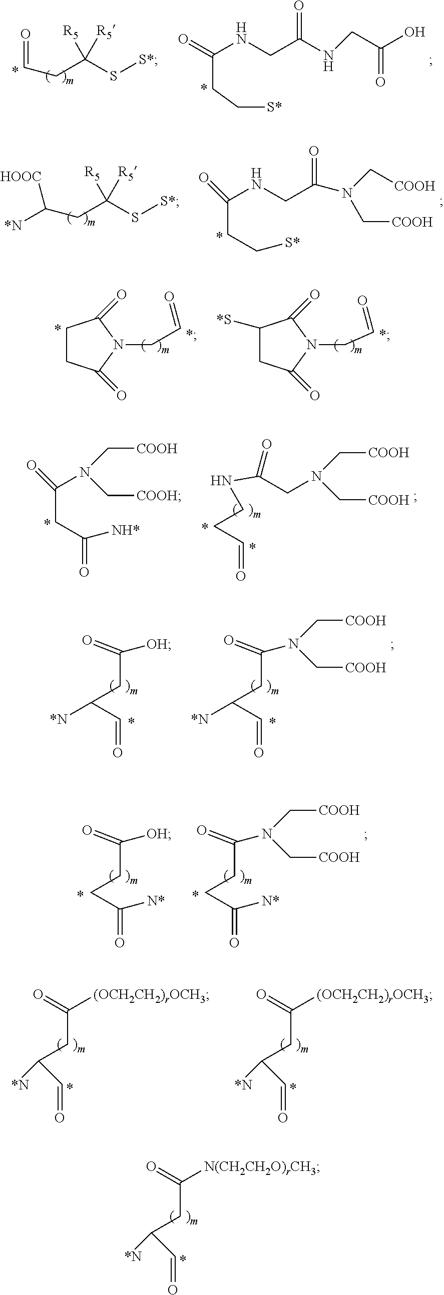 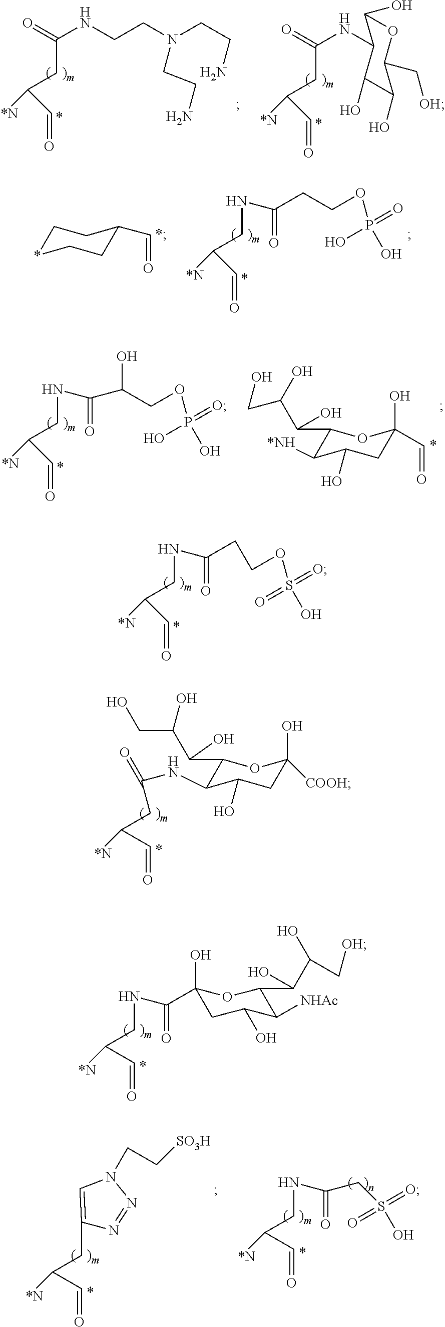 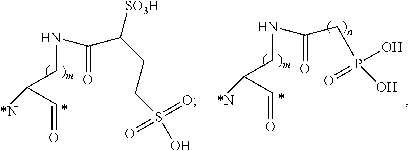
wherein a (*) atom is a point of attachment of additional spacer R₁ or releasable linker, cytotoxic agent, and/or binding molecule; X¹, Y¹, U¹, R₁, R₅, R₅′ are defined as above; r is 0-100; m and n are 0-6 independently;
wherein L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or a natural or unnatural peptide having 1-8 natural or unnatural amino acid units;
wherein L₁ or L₂ may be a releasable linker;
wherein the releasable linker (L₁ or L₂) comprises:
—(CR₅R₆)ₘ(Aa)r(CR₇R₈)ₙ(OCH₂CH₂)ₜ—, —(CR₅R₆)ₘ(CR₇R₈)ₙ(Aa)ᵣ(OCH₂CH₂)ₜ—, -(Aa)ᵣ-(CR₅R₆)ₘ(CR₇R₈)ₙ(OCH₂CH₂)ᵣ—, —(CR₅R₆)ₘ(CR₇R₈)ₙ(OCH₂CH₂)ᵣ(Aa)-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa) (CR₉R₁₀)ₙ—(OCH₂CH₂)ᵣ—, —(CR₅R₆)ₘ-phenyl-CO(Aa)ₜ(CR₇R₈)ₙ—, —(CRSR₆)ₘ-furyl-CO(Aa)ₜ(CR₇R₈)ₙ—, —(CR₅R₆)ₘ-oxazolyl-CO(Aa)ᵣ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ-(Aa)ₜphenyl-, —(CR₅R₆)ₘ-(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(CR₅SR₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₁₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)timidazolyl-CO—(CR₇R₈)ₙ—, —K(CR₅R₆)ₜmorpholino-CO(Aa)(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R)ₘ(Aa)ₜphenyl, —K—(CR₅R₆)ₘ—(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein in, Aa, m, n, R₃, R₄, and R₅ are described above; t and r are 0-100 independently; R₆, R₇, and R₈ are independently chosen from −1; halide; C₁˜C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group;
wherein the structures of the components of the linker L₁ and L₂ can be: 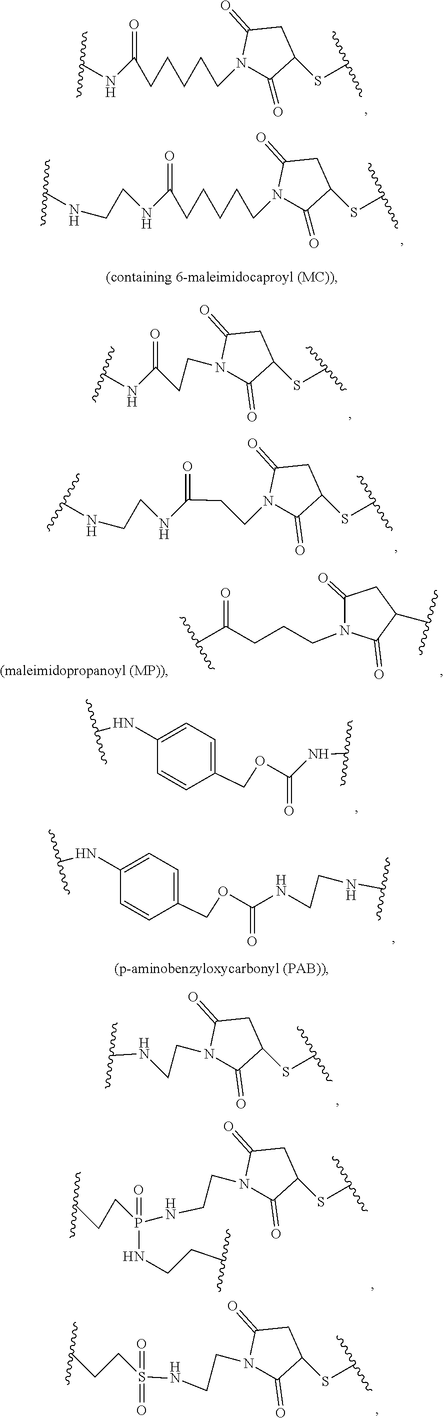 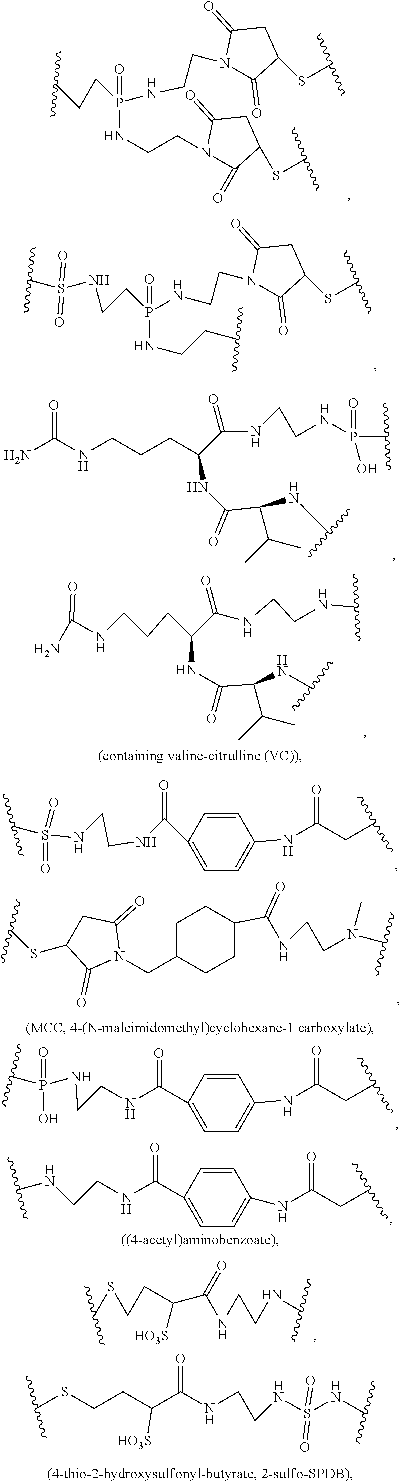 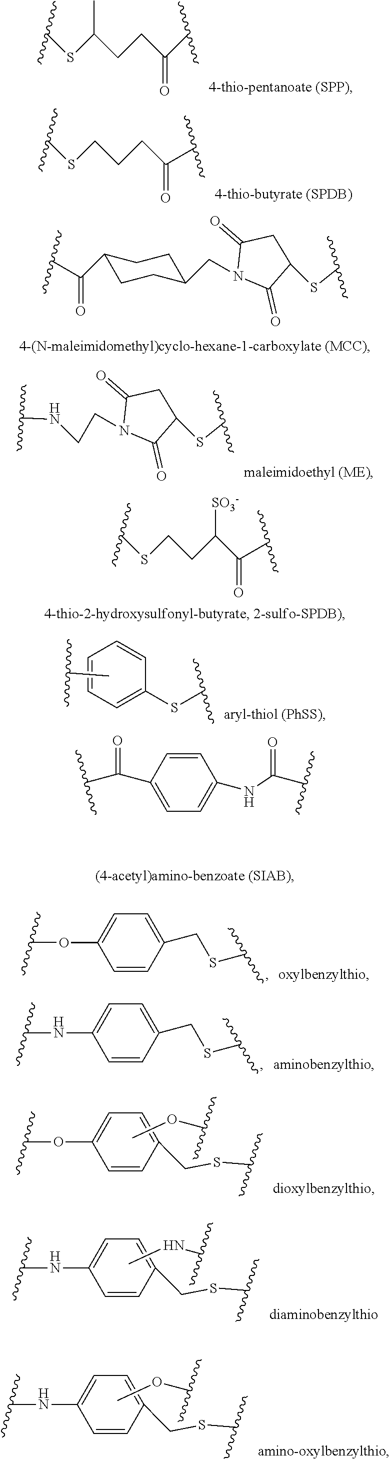 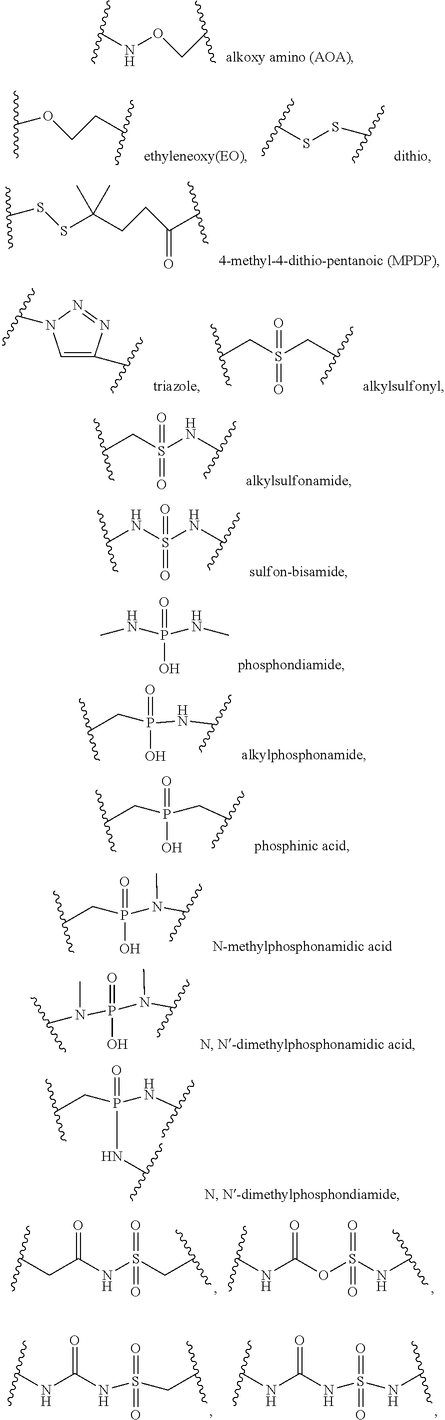 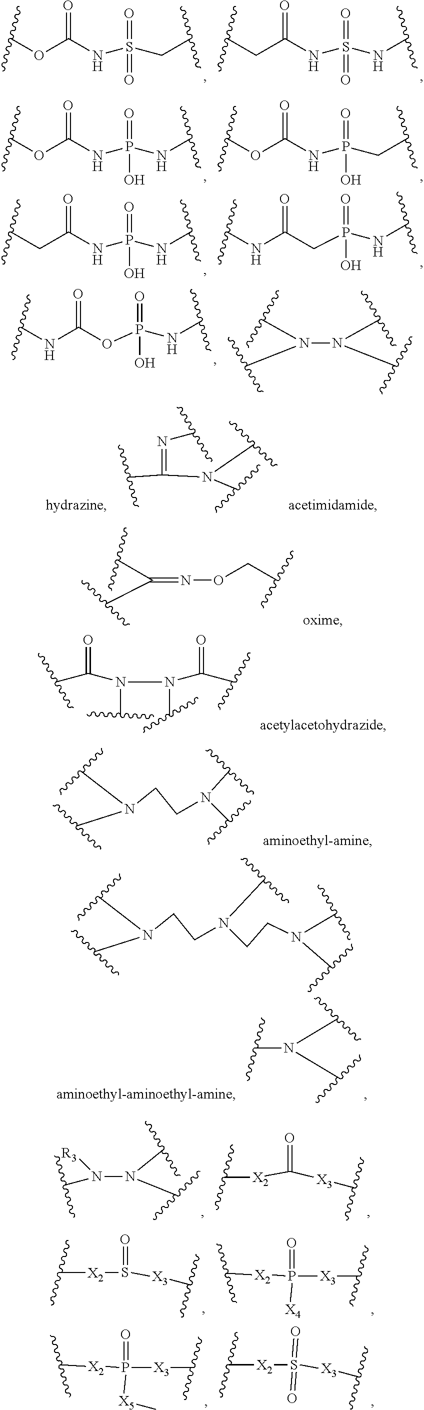 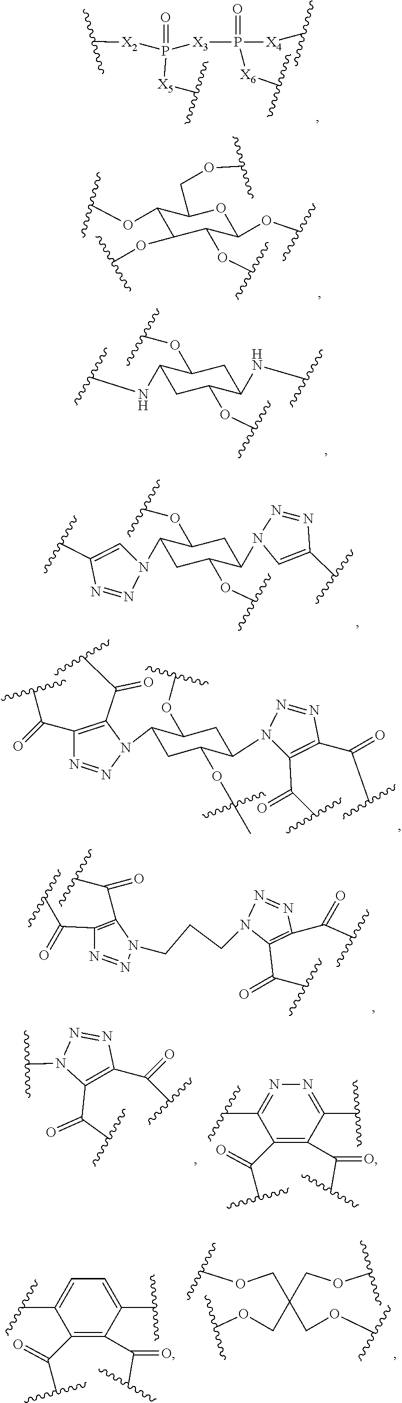 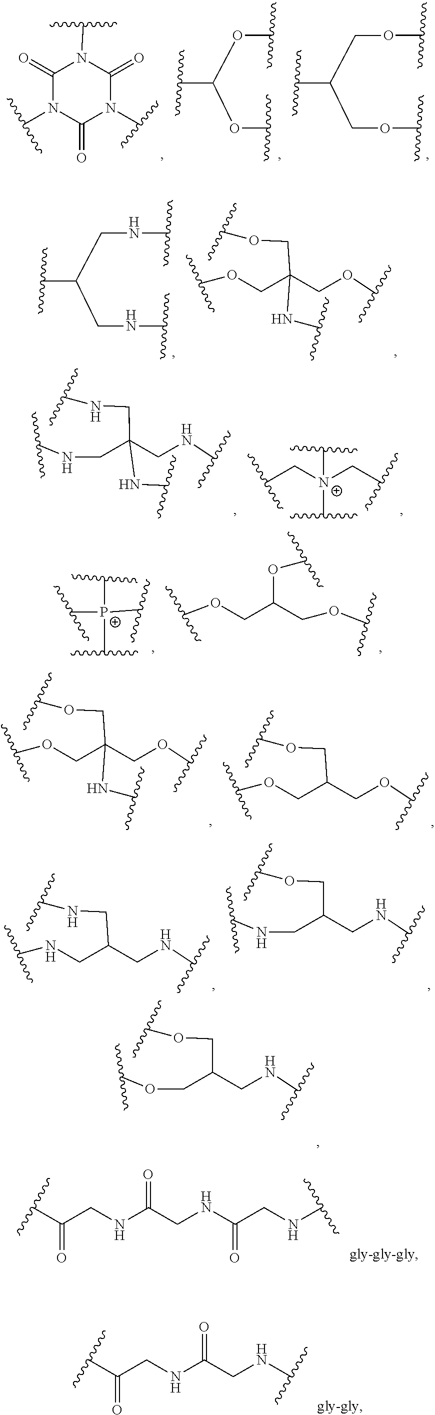 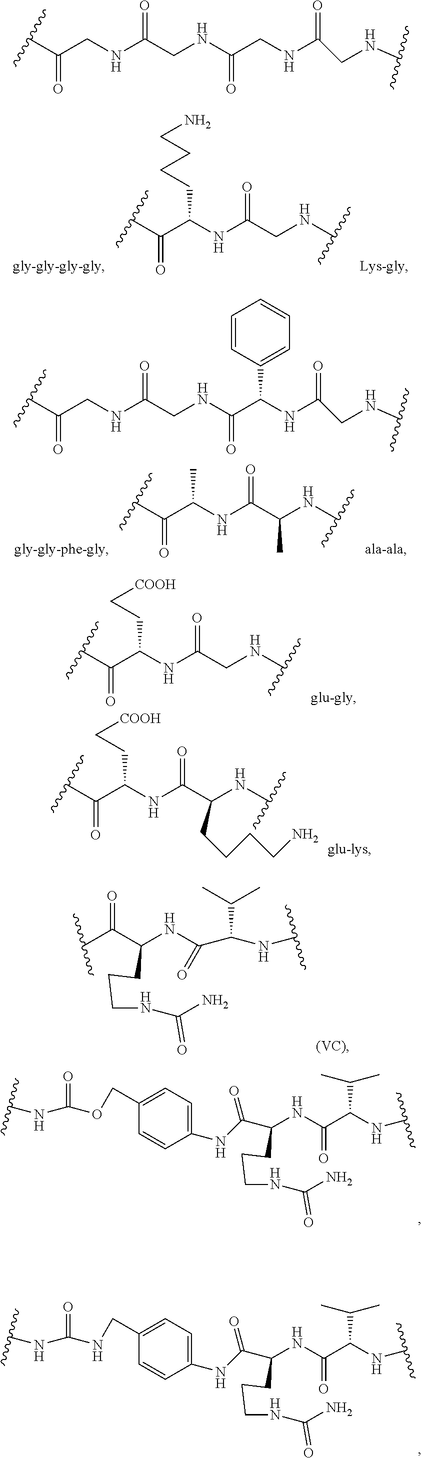 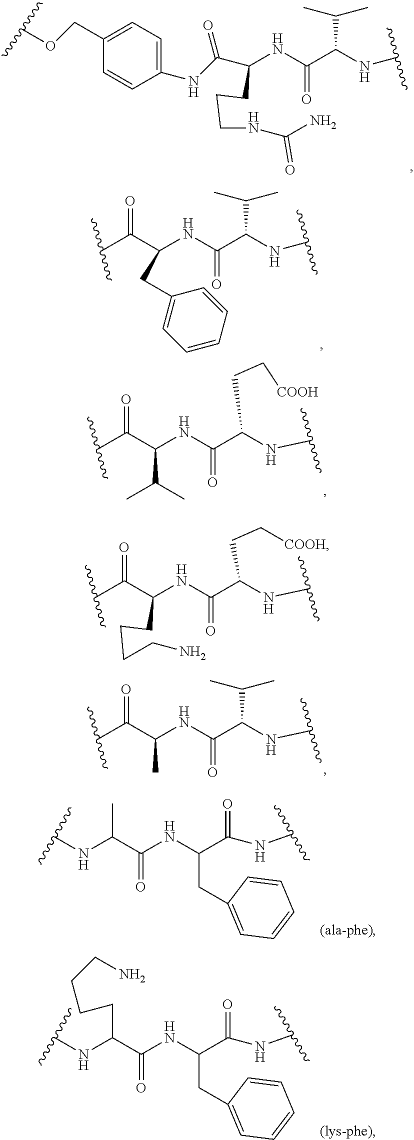
or a combination of two or more of the above; wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or polyethyleneoxy unit of formula (OCH₂CH₂)ₚ or (OCH₂CH(CH₃))ₚ, wherein p is an integer from 0 to about 1000, or a combination of two or more of the above groups;
wherein L₁, L₂, X₁, X₂, X₃, X₁′, X₂′ and X₃′ can further be independently absent;
wherein E₁ is a joint group that linked two thiol reactive groups of Lv₁ and Lv₂, and E₁ is selected from CH, CH₂, NH, NHNH, N(R₃), N(R₃)N(R₃′), N═N, N—N, P, P(═O), S, Si, C₂-C₈ alkyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; a peptide containing 1-4 units of aminoacids; or one of the following structures: 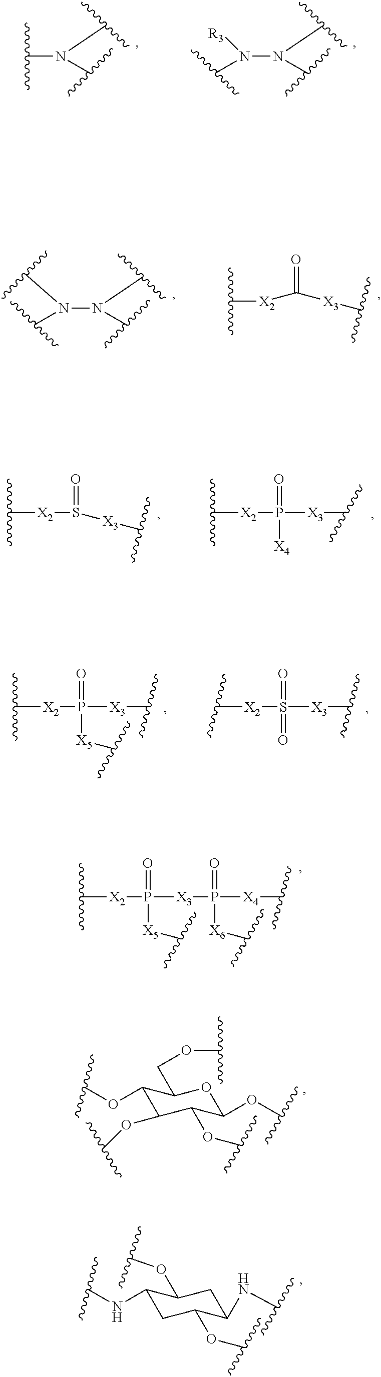 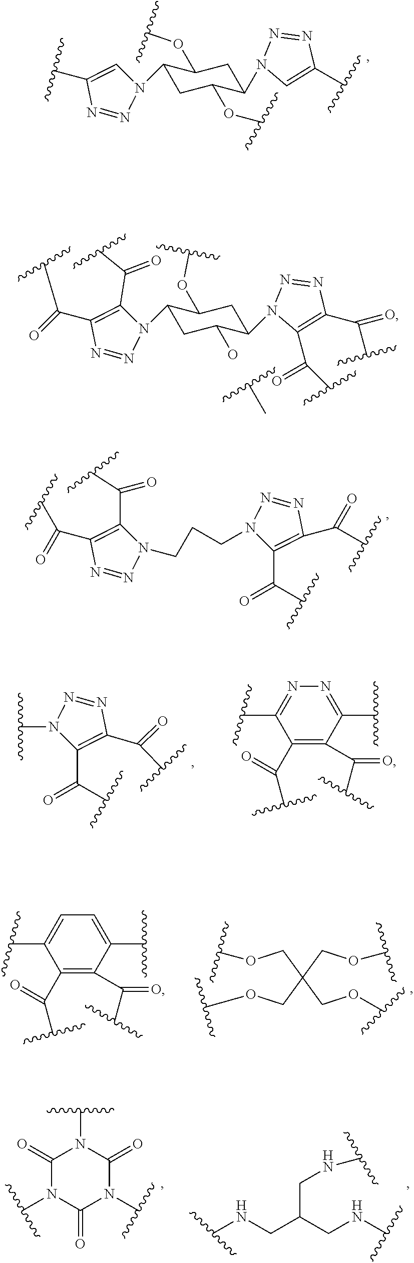 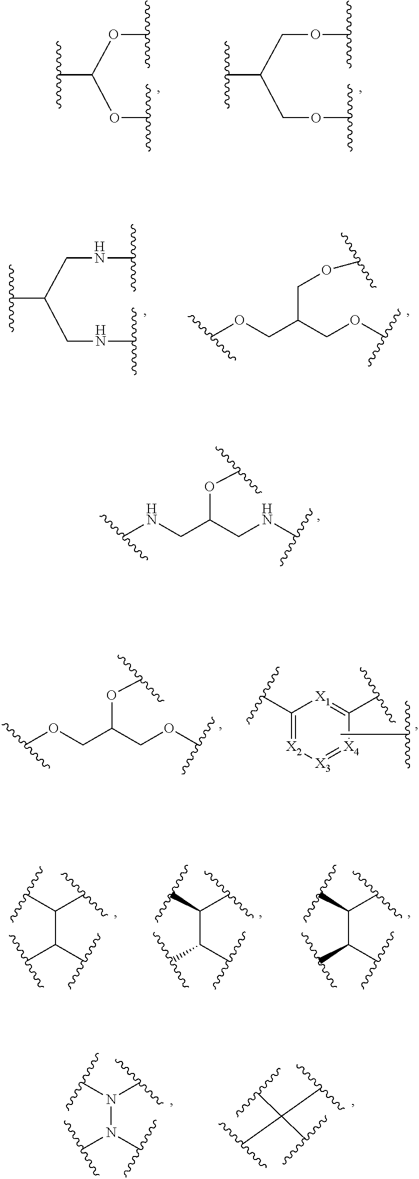 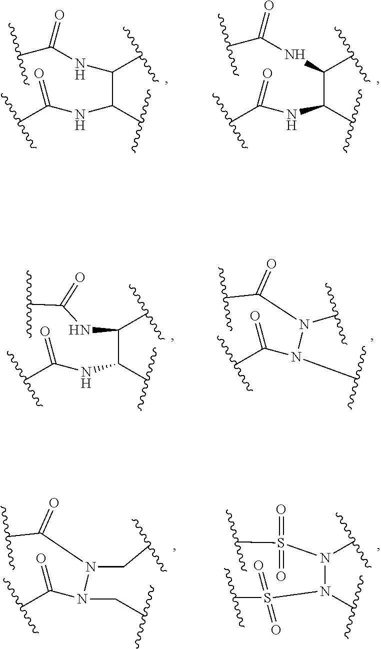
wherein [symbol] is a site of linkage;
wherein D₁ and D₂ are a cytotoxic drug, or a therapeutic drug, an immunotherapeutical short antibody-like protein, a function molecule for enhancement of binding or stabilization of the cell-binding antibody-like protein agent, a cell-surface receptor binding ligand, an antibody fragment, siRNA or DNA molecule, and are selected from:
1) Chemotherapeutic agents:
a) Alkylating agents: nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its synthetic analogues adozelesin, carzelesin and bizelesin); Duocarmycin (including the synthetic analogues, KW-2189 and CBI-TMI); Benzodiazepine dimers (including dimers of pyrrolobenzodiazepine (PBD) or tomaymycin, indolinobenzodiazepines, imidazobenzothiadiazepines, or oxazolidino-benzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines: benzodopa, carboquone, meturedopa, and uredopa; ethylenimines and methylamelamines including altretamine, triethylenemel-amine, trietylenephosphoramide, triethylenethio-phosphaoramide and trimethylolomel-amine];
b) Plant Alkaloids: Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermo-lide, bryostatins, dolostatins, auristatins, tubulysins, cephalostatins; pancratistatin; a sarcodictyin; spongistatin;
c) DNA Topoisomerase Inhibitors: Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C);
d) Anti-metabolites: [Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, frolinic acid;
e) Hormonal therapies: Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human antibody-like proteins containing a TNF domain)];
f) Kinase inhibitors: BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). antibiotics, such as the enediyne antibiotics: calicheamicins:calicheamicin γ1, δ1, α1 and β1; dynemicin: dynemicin A and deoxydynemicin; esperamicin, kedarcidin, C-1027, maduropeptin, neocarzinostatin chromophore and related chromoprotein enediyne antiobiotic chromomophores, aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
f) Polyketides (acetogenins): bullatacin and bullatacinone; gemcitabine, epoxomicins, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors: Lovastatin, Dopaminergic neurotoxins: 1-methyl-4-phenylpyridinium ion, Cell cycle inhibitors: staurosporine, Actinomycins: Actinomycin D, dactinomycin, Bleomycins: bleomycin A2, bleomycin B2, peplomycin, Anthracyclines: daunorubicin, doxorubicin (adriamycin), idarubicin, epirubicin, pirarubicin, zorubicin, mtoxantrone, MDR inhibitors: verapamil, Ca²+ATPase inhibitors: thapsigargin, Histone deacetylase inhibitors: Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A; Thapsigargin, Celecoxib, glitazones, epigallocatechin gallate, Disulfiram, Salinosporamide A.; Anti-adrenals: aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine, razoxane; rhizoxin; sizofiran; spirogermanium; tenuazonic acid; triaziquone; 2, 2′, 2″-trichlorotriethylamine; trichothecenes: T-2 toxin, verrucarin A, roridin A and anguidine; urethane, siRNA, antisense drugs, and a nucleolytic enzyme;
2) An anti-autoimmune disease agent selected from the group consisting of cyclosporine, cyclosporine A, aminocaproic acid, azathioprine, bromocriptine, chlorambucil, chloroquine, cyclophosphamide, corticosteroids: amcinonide, betamethasone, budesonide, hydrocortisone, flunisolide, fluticasone propionate, fluocortolone danazol, dexamethasone, Triamcinolone acetonide, beclometasone dipropionate, DHEA, enanercept, hydroxychloroquine, infliximab, meloxicam, methotrexate, mofetil, mycophenylate, prednisone, sirolimus, and tacrolimus.
3) An anti-infectious disease agent:
a) Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b) Amphenicols:azidamfenicol, chloramphenicol, florfenicol, thiamphenicol;
c) Ansamycins: geldanamycin, herbimycin;
d) Carbapenems: biapenem, doripenem, ertapenem, imipenem/cilastatin, meropenem, panipenem;
e) Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f) Glycopeptides: bleomycin, vancomycin (oritavancin, telavancin), teicoplanin (dalbavancin), ramoplanin;
g) Glycylcyclines;
h). β-Lactamase inhibitors: penam (sulbactam, tazobactam), clavam (clavulanic acid);
i). Lincosamides: clindamycin, lincomycin;
j) Lipopeptides: daptomycin, A54145, calcium-dependent antibiotics (CDA);
k) Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l) Monobactams: aztreonam, tigemonam;
m) Oxazolidinones: linezolid;
n) Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o) Polypeptides: bacitracin, colistin, polymyxin B;
p) Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q) Streptogramins: pristinamycin, quinupristin/dalfopristin);
r) Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s) Steroid antibacterials;
t) Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines;
u) antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors: fosfomycin, nitrofurantoin, paclitaxel, platensimycin, pyrazinamide, quinupristin/dalfopristin, rifampicin (rifampin), tazobactam tinidazole, uvaricin;
4) Anti-viral drugs:
a) Entry/fusion inhibitors: aplaviroc, maraviroc, vicriviroc, gp41 (enfuvirtide), PRO 140, CD4 (ibalizumab);
b) Integrase inhibitors: raltegravir, elvitegravir, globoidnan A;
c) Maturation inhibitors: bevirimat, vivecon;
d) Neuraminidase inhibitors: oseltamivir, zanamivir, peramivir;
e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (3′-fluoro-2′, 3′-dideoxythymidine (FLT) and 3′-fluoro-2′, 3′-dideoxyguanosine (FLG), fomivirsen, ganciclovir, idoxuridine, lamivudine (3TC), 1-nucleosides (p1-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f) Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g) Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h) anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
5) radioisotopes (radionuclides): ³H, ¹¹C, ¹⁴C, ¹⁸F, ³²p, ³⁵S, ⁶⁴Cu, ⁶⁸Ga, ⁸⁶Y ⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi;
6) a pharmaceutically acceptable salt, acid or derivative of any of the above drugs.

28. The process according to claim 27, wherein the thiol groups in the antibody or antibody-like protein generated in step (a) simultaneously or sequentially react with the drug/linker complex of formula (I), (II) or (III) independently to form a conjugate of formula (V), (VI), or (VII) as represented below: 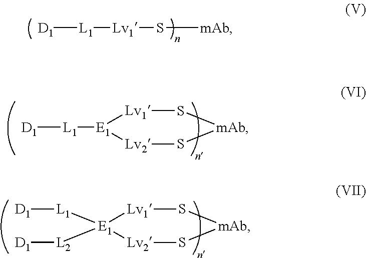
wherein n is 1-20; n′ is 1-10; D₁, D₂, L₁, L₂, and E₁ are the same as described in claim 27; S (sulfur) is generated from the reduction of disulfide bonds in the antibody or antibody-like protein; mAb is the antibody or antibody-like protein;
wherein Lv₁′ and Lv₂′ independently have one of the following structures: 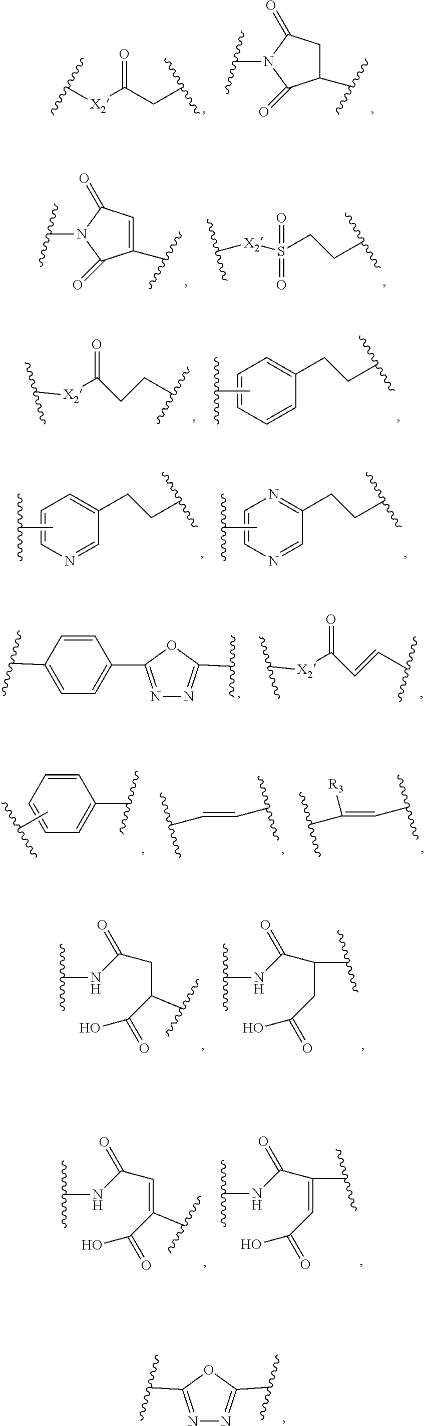
wherein X₂′ and R₃ are defined the same as in claim 27;
wherein in the formula (VI) and formula (VII) the moities of, 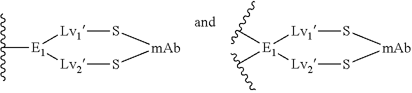
are independently selected from: 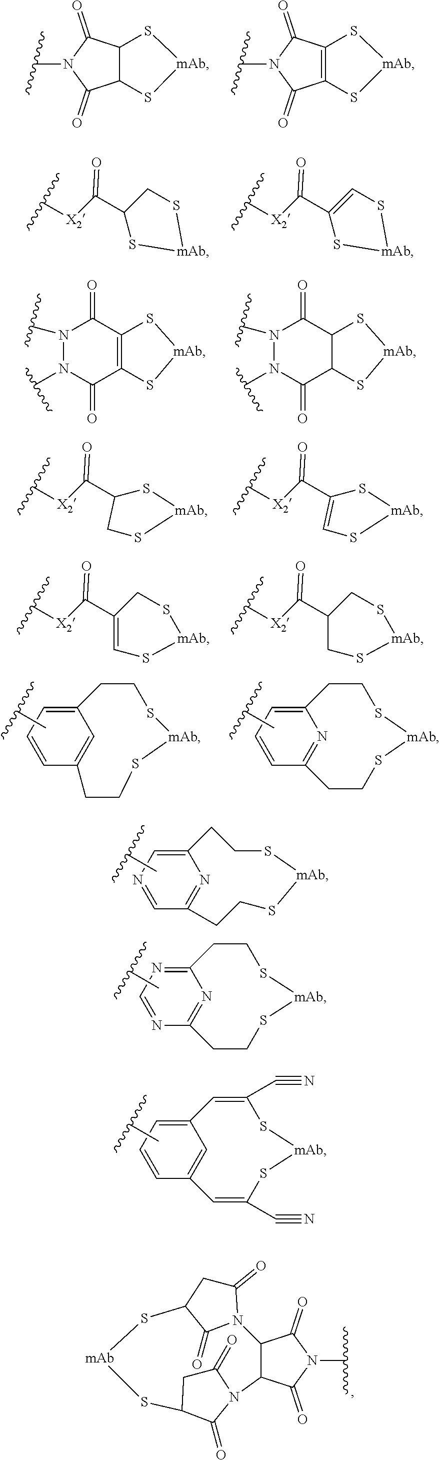 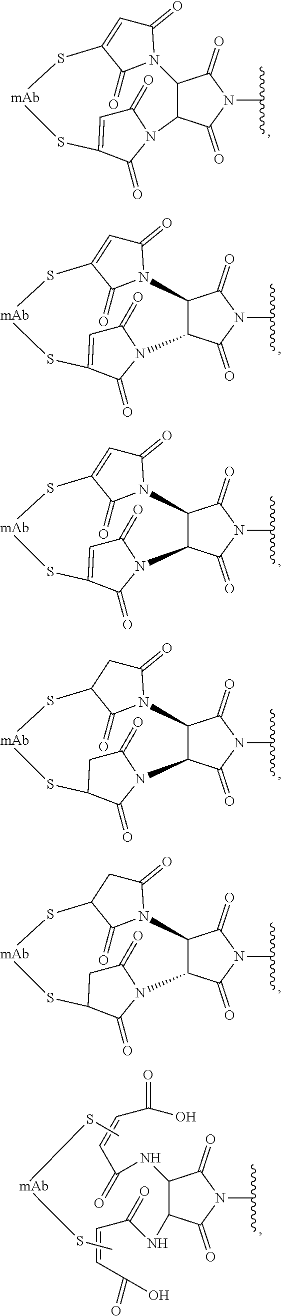 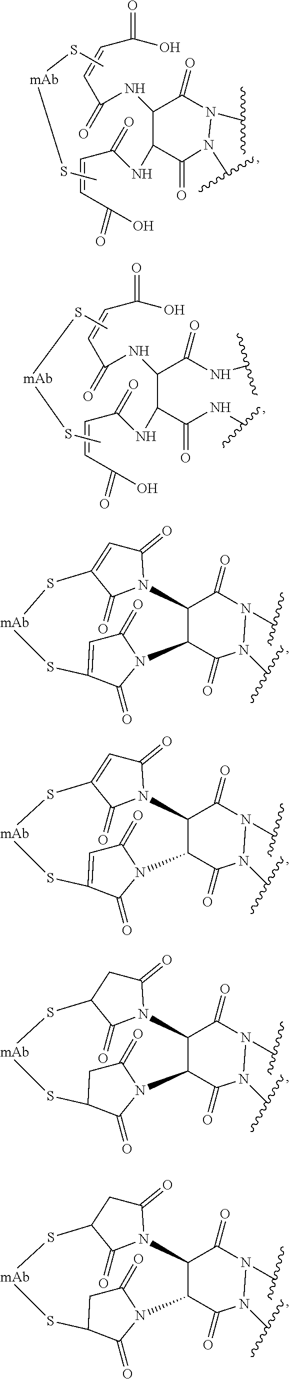 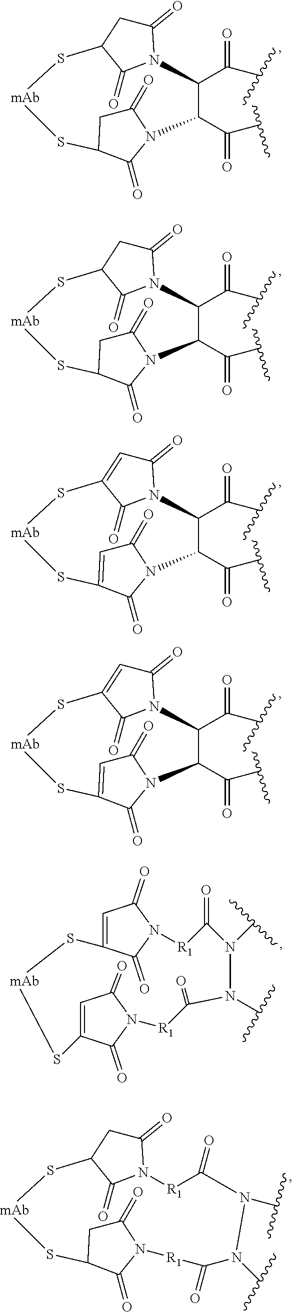 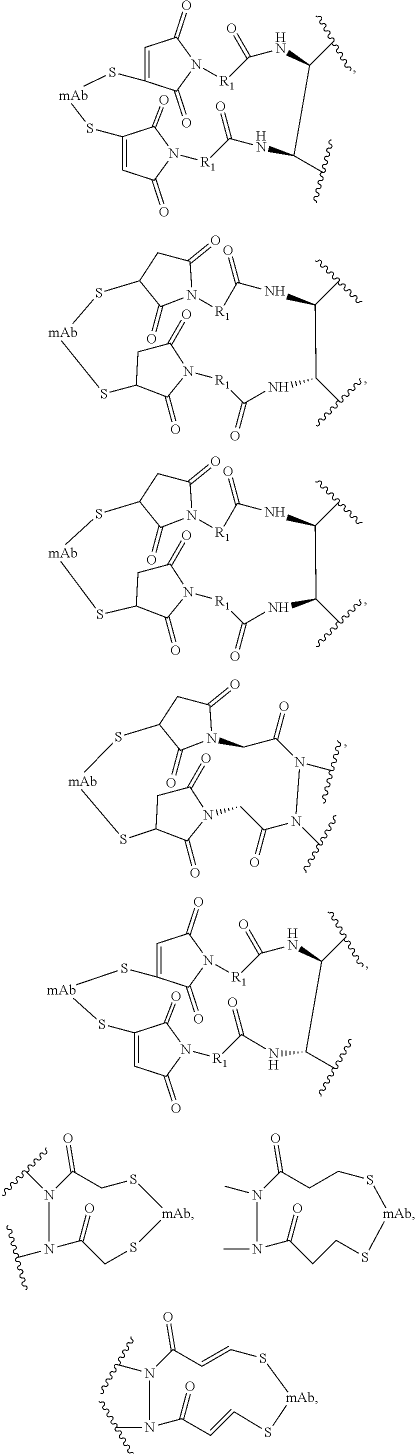 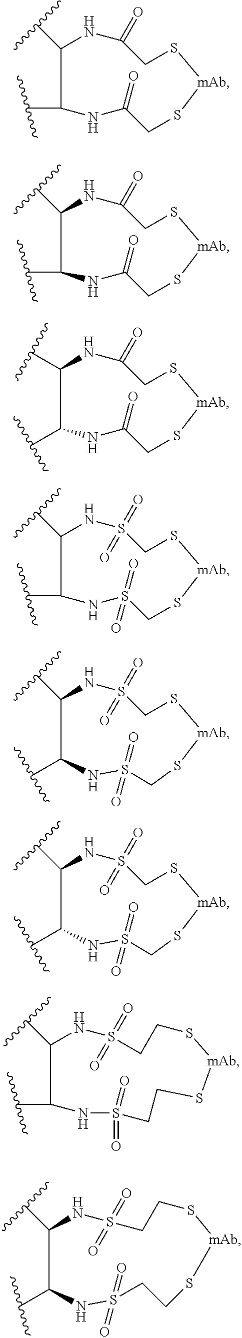 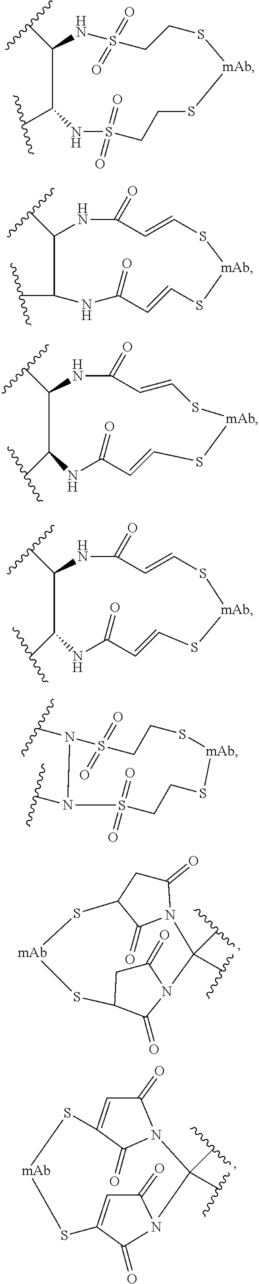 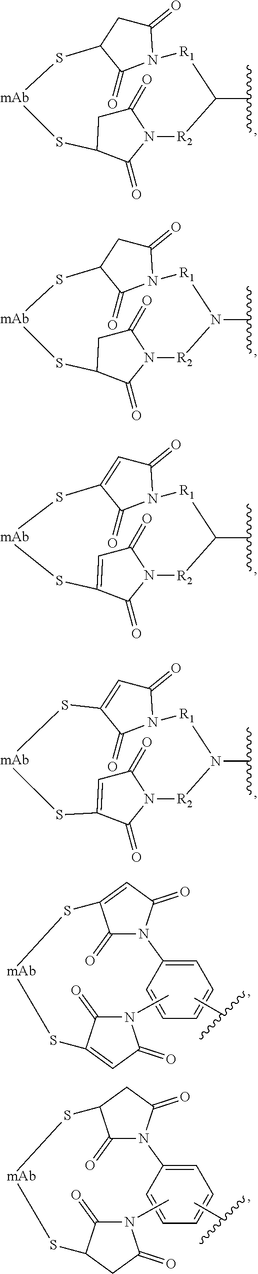 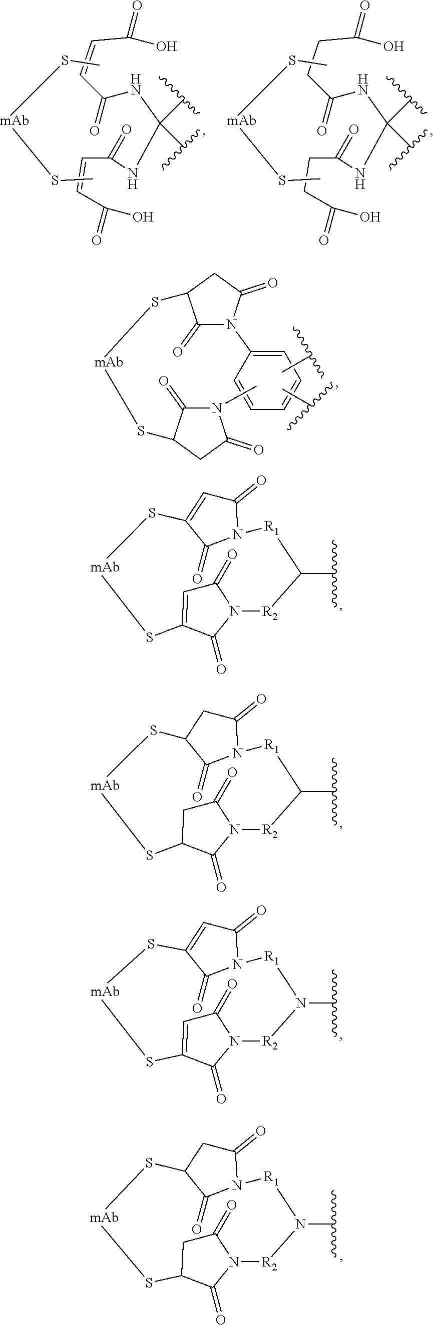 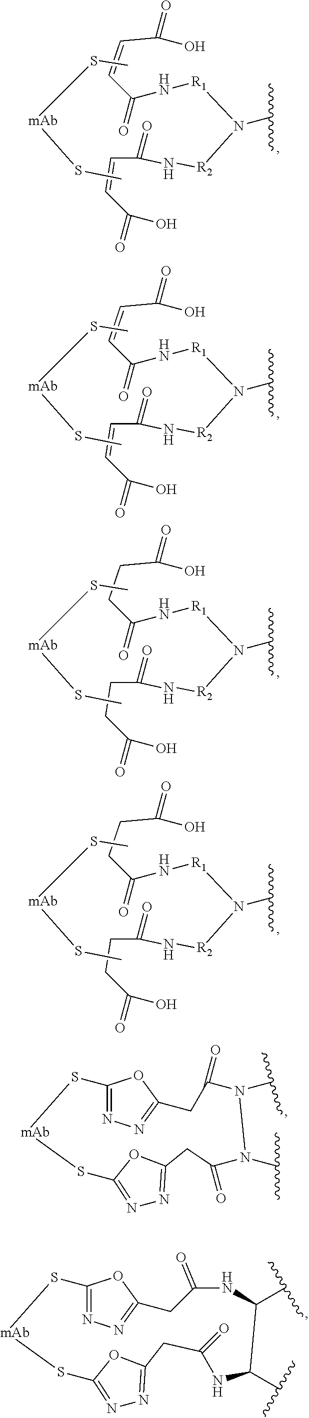 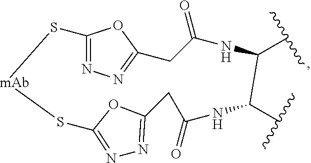
wherein R₁, R₂, X₂ are the same as defined above; mAb is the antibody or antibody-like protein.

29. The process according to claim 23, wherein the thiol groups in the antibody or antibody-like protein generated in step (a) simultaneously or sequentially react with a condensation compound having structural formula (VIII), (IX) or (X) as illustrated below and independently with a drug, D₁ and/or D₂, to form a conjugate of formula (V), (VI), or (VII): 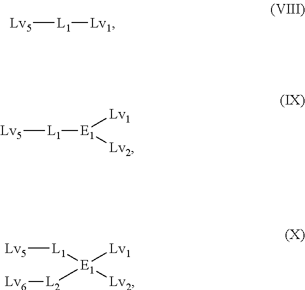
wherein L₁ for Formula (I), (II) and (III) and L₂ are, the same or different, independently O, NH, S, NHNH, N(R₃), N(R₃)N(R₃′), polyethyleneoxy unit of formula (OCH₂CH₂)ₚOR₃, (OCH₂CH(CH₃))ₚOR₃, NH(CH₂CH₂O)ₚR₃, or NH(CH₂CH(CH₃)O)ₚR₃, N[(CH₂CH₂O)ₚR₃][(CH₂CH₂O)ₚ′R₃′], (OCH₂CH₂)ₚC₀₀R₃, CH₂CH₂(OCH₂CH₂)ₚC₀₀R₃, wherein p and p′ are an integer independently selected from 0 to about 1000; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; wherein R₃ and R₃′ are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or 1-8 natural or unnatural amino acids; or polyethyleneoxy unit of formula (OCH₂CH₂)ₚ or (OCH₂CH(CH₃))ₚ, wherein p is an integer from 0 to about 1000, or a combination of two or more of the above;
wherein L₁ or L₂ may contain a self-immolative or a non-self-immolative component, peptidyl unit, a hydrazone bond, a disulfide, an ester, an oxime, an amide, or a thioether bond, the self-immolative unit is an aromatic compound that is electronically similar to a para-aminobenzylcarbamoyl (PAB) group, and is selected from the group consisting of 2-aminoimidazol-5-methanol compounds, heterocyclic PAB compounds, beta-glucuronide, and ortho or para-aminobenzylacetals;
wherein the self-immolative linker component has one of the following structures: 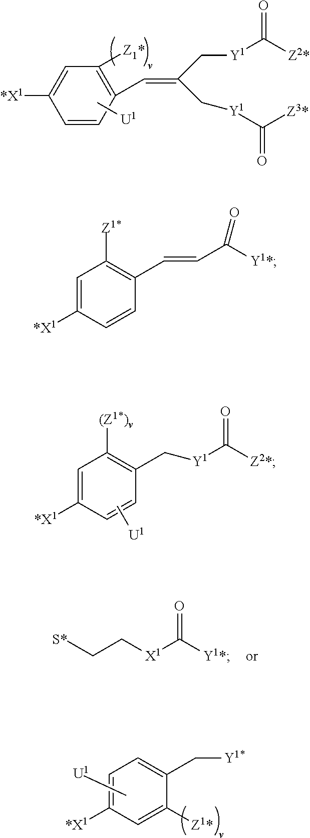
wherein a (*) atom is a point of attachment of additional spacer or releasable linker unit, or a cytotoxic agent, and/or a cell binding molecule (CBA); X¹, Y¹, Z² and Z³ are independently NH, O, or S; Z¹ is independently H, NH, O or S; v is 0 or 1; Uⁱ is independently H, OH, C₁-C₆ alkyl, (OCH₂CH₂)ₙF, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′) wherein R₅ and R₅′ are as defined above;
wherein the non-self-immolative linker component is one of the following structures: 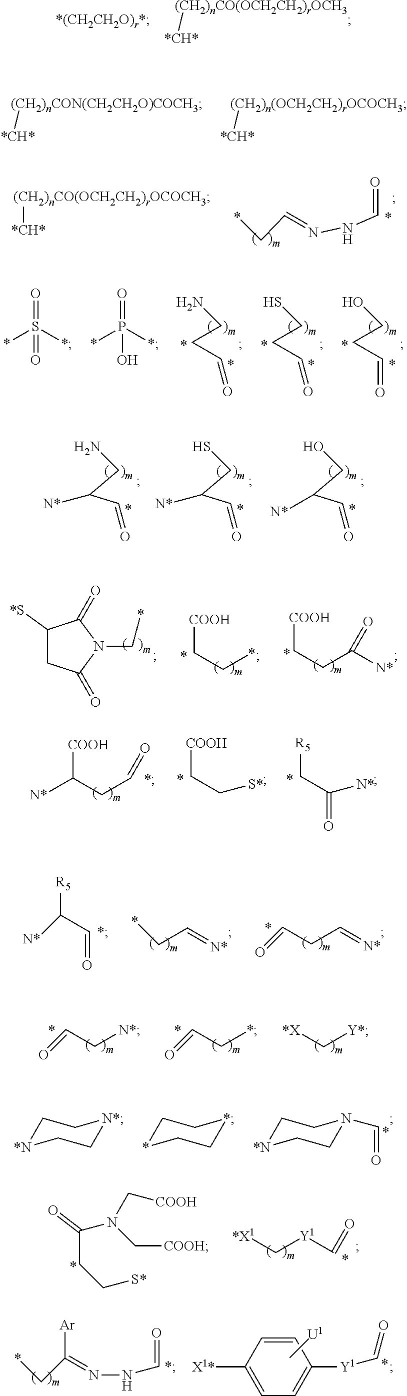 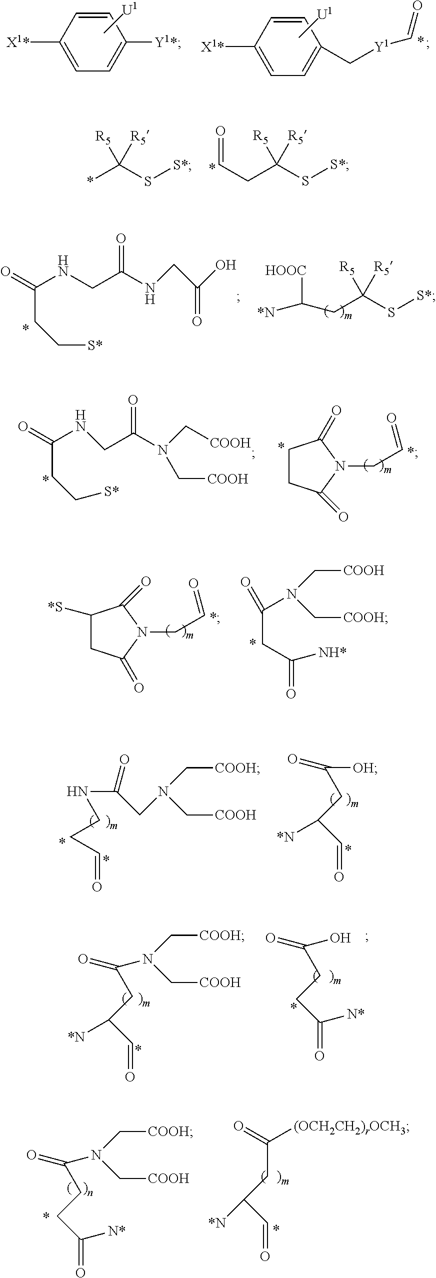 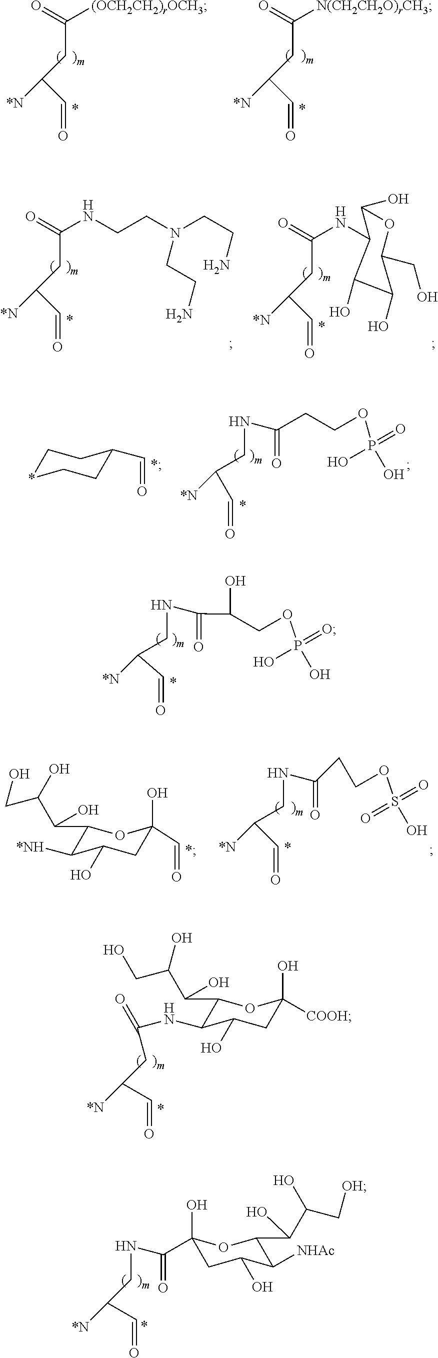 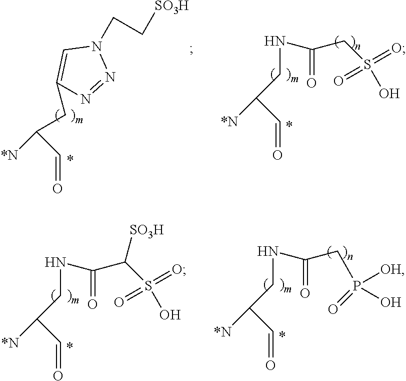
wherein a (*) atom is a point of attachment of additional spacer R₁ or releasable linker, cytotoxic agent, and/or binding molecule; X¹, Y¹, U¹, R₁, R₅, R₅′ are defined as above; r is 0-100; m and n are 0-6 independently;
wherein L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or a natural or unnatural peptide having 1-8 natural or unnatural amino acid units;
wherein L₁ or L₂ may be a releasable linker;
wherein the releasable linker (L₁ or L₂) comprises:
—(CR₅R₆)ₘ(Aa)ᵣ(CR₇R₈)ₙ(OCH₂CH₂)ₜ—, —(CR₅R₆)ₘ(CR₇R₈)ₙ(Aa)ᵣ(OCH₂CH₂)ₜ—, - (Aa)ᵣ-(CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)ₜ-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂C₁₂)ᵣ—, —(CR₅R₆)ₘ(CO)(Aa) (CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CRR)ₘ-oxazolyl-CO(Aa)ₜ(CR₇R₈)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)-imidazolyl-CO—(CR₇R₈)ₙ—, —(CR₅R₆)ₜ-morpholino-CO(Aa)ₜ-(CR₇R₈)ₙ—, —(CR₅R₆)ₜpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ—(Aa)ₜphenyl-, —(CR₅R₆)ₘ—(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(C R₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₁₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OC₁₂C₁₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OCH₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)timidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)—(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ—(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, n, R₃, R₄, and R₅ are described above; t and r are 0-100 independently; R₆, R₇, and R₈ are independently chosen from H; halide; C₁˜C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group;
wherein the structures of the components of the linker L₁ and L₂ can be: 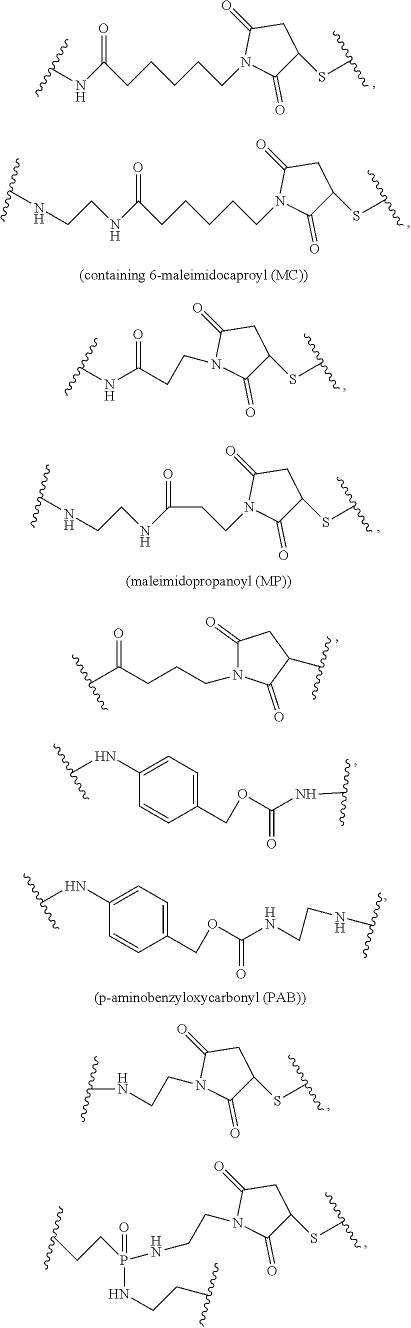 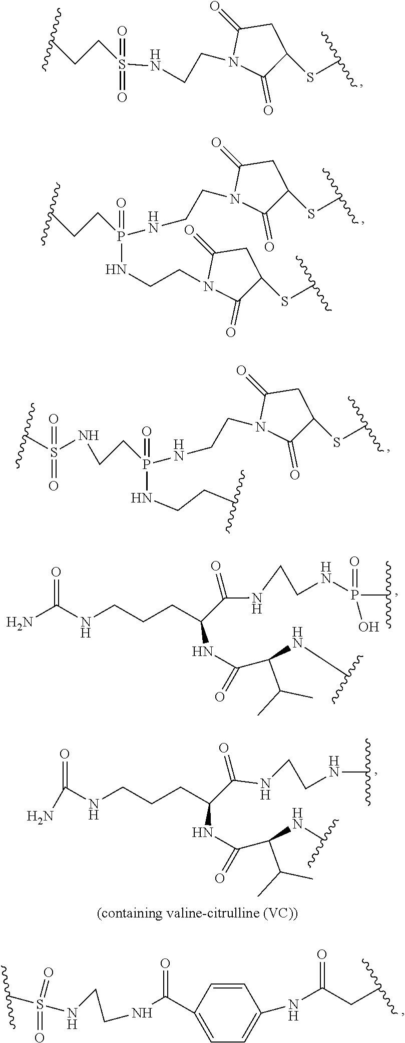 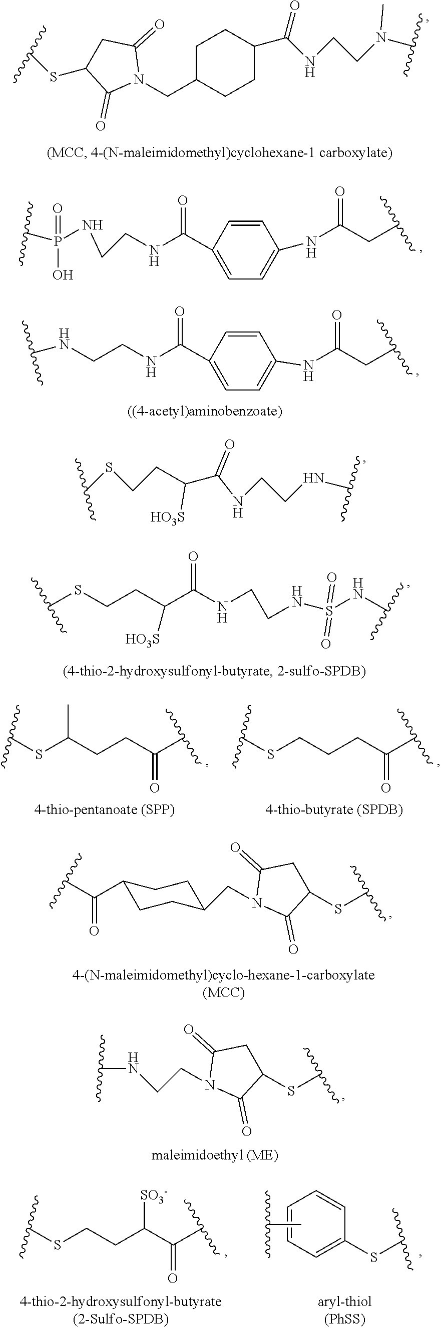 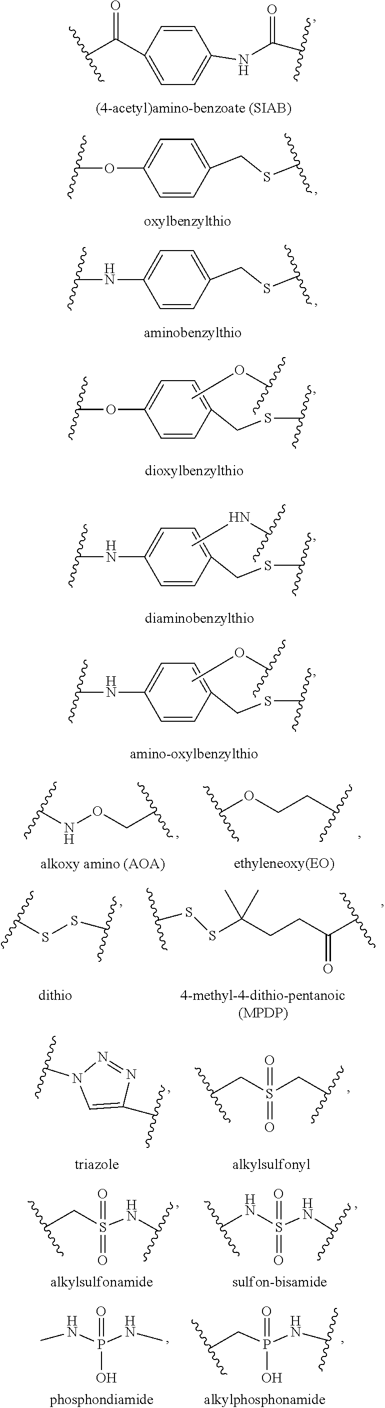 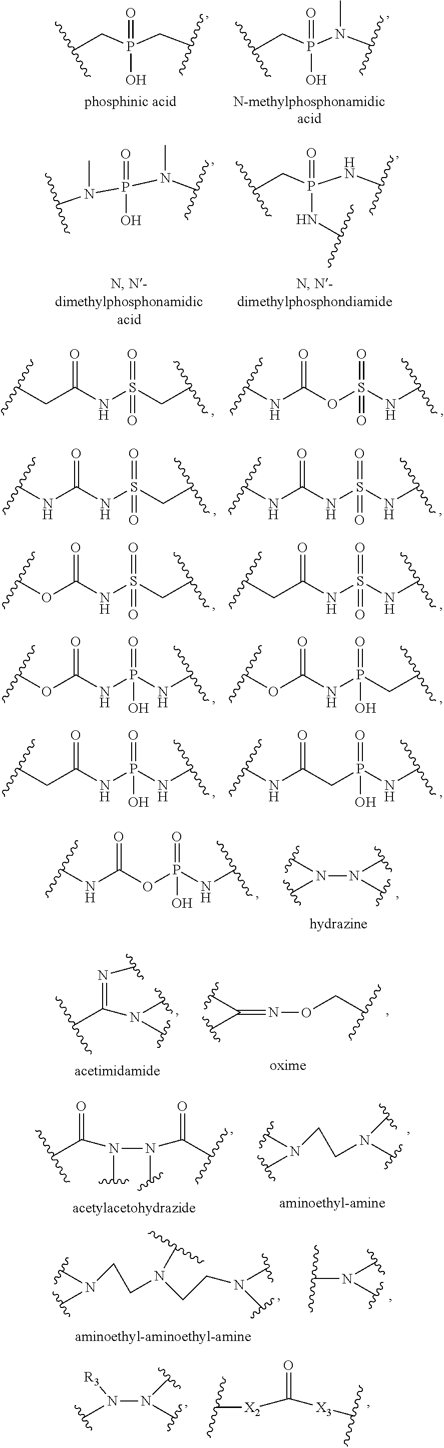 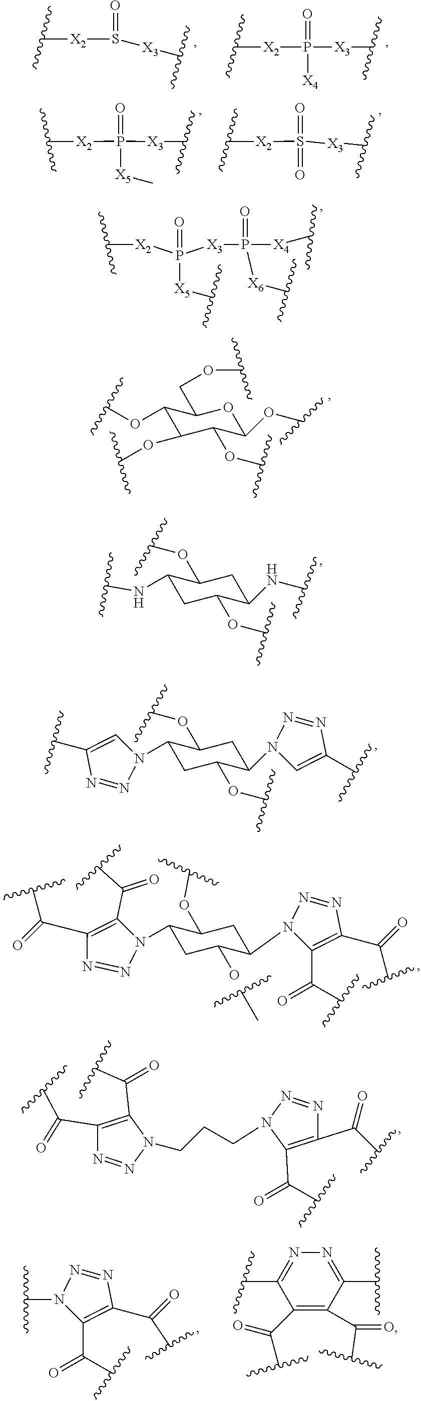 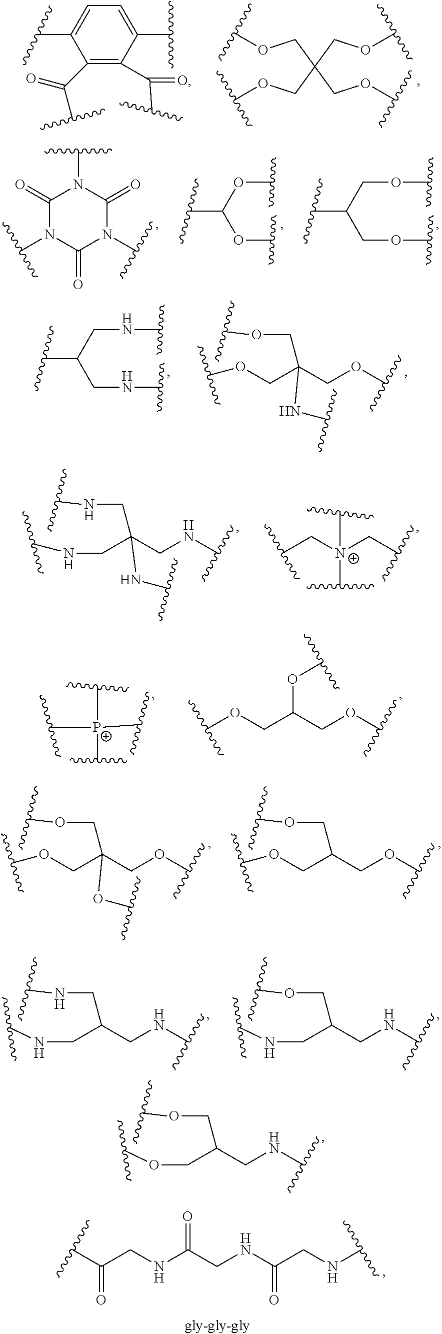 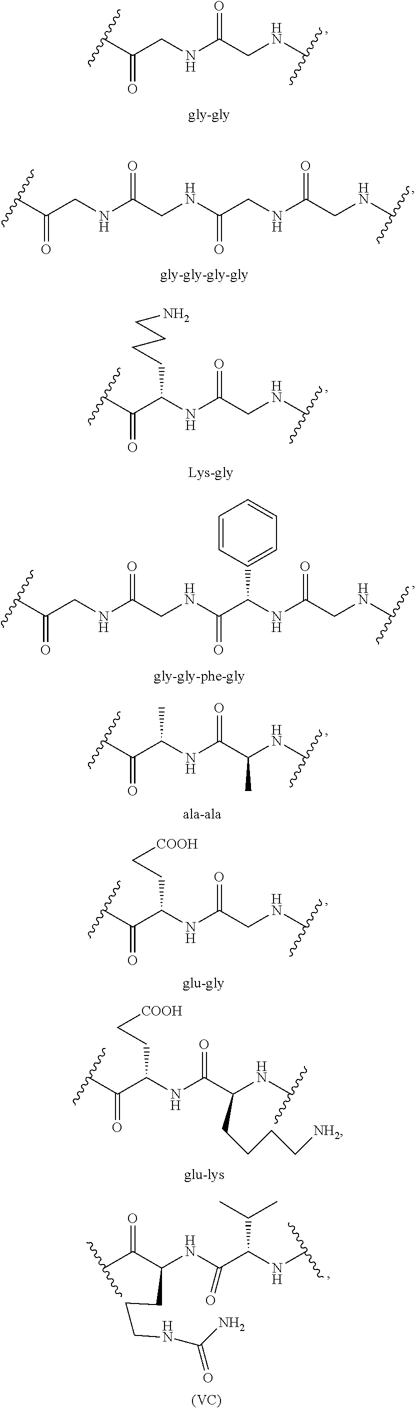 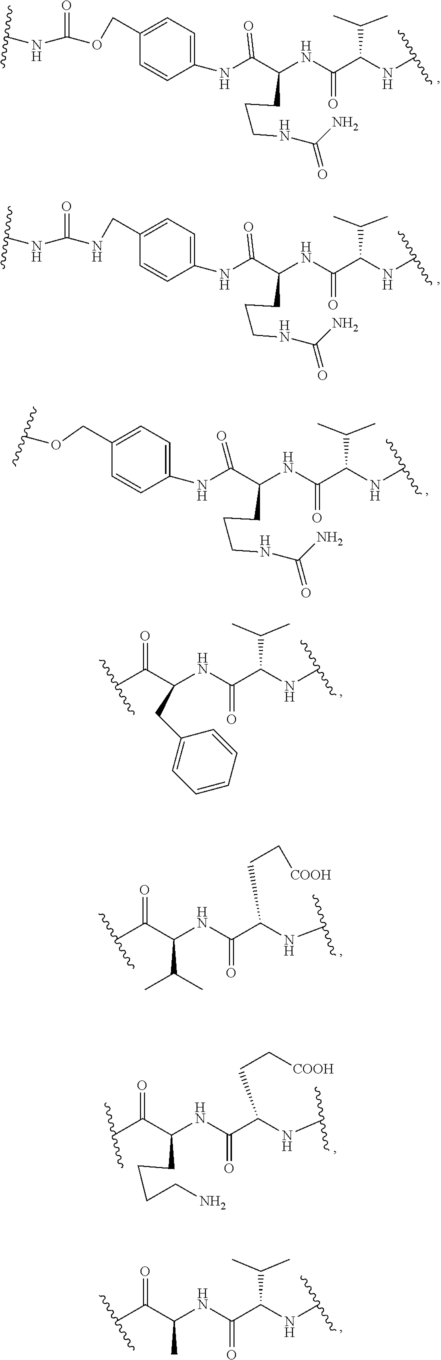 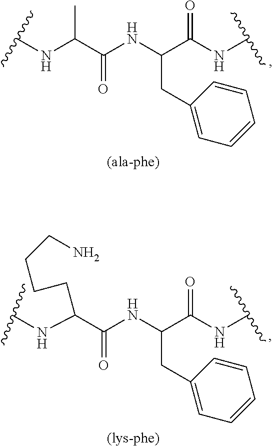 [SMILES: *NC(=O)C(Cc1ccccc1)NC(=O)C(C)N* ; *NC(=O)C(Cc1ccccc1)NC(=O)C(CCCCN)N*]
or a combination of two or more of the above; wherein [symbol] is the site of linkage; X₂, X₃, X₄, X₅, or X₆, are independently selected from NH; NHNH; N(R₁₂); N(R₁₂)N(R₁₂); O; S; C₁-C₆ of alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or polyethyleneoxy unit of formula (OCH₂CH₂)ₚ or (OCH₂CH(CH₃))ₚ, wherein p is an integer from 0 to about 1000, or a combination of two or more of the above groups;
wherein L₁, L₂, X₁, X₂, X₃, X₁′, X₂′ and X₃′ can further be independently absent;
wherein E₁ is a joint group that linked two thiol reactive groups of Lv₁ and Lv₂, and E₁ is selected from CH, CH₂, NH, NHNH, N(R₃), N(R₃)N(R₃′), N═N, N—N, P, P(═O), S, Si, C₂-C₈ alkyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; a peptide containing 1-4 units of aminoacids; or one of the following structures: 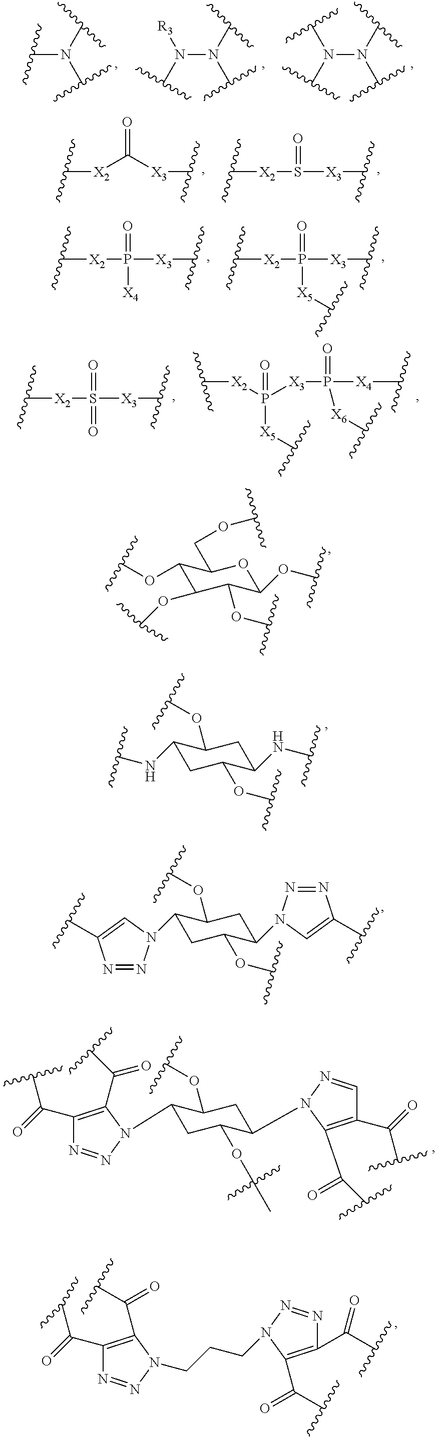 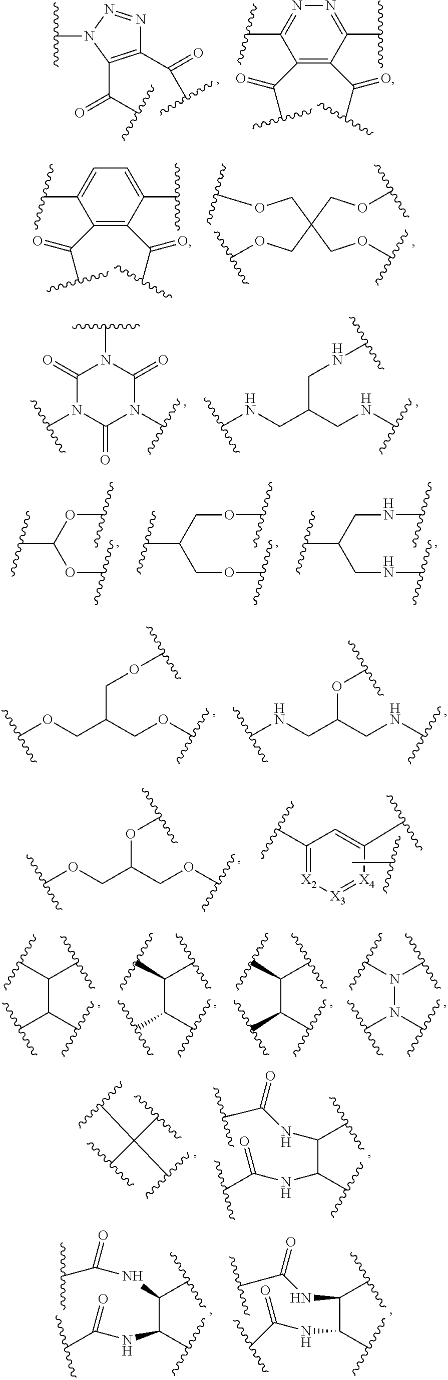 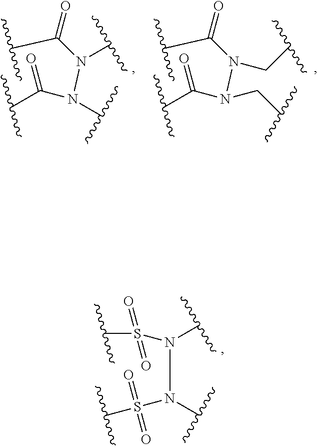
wherein [symbol] is a site of linkage;
Lv₁ and Lv₂ are a thiol reaction group, and are independently selected from the group consisting of: 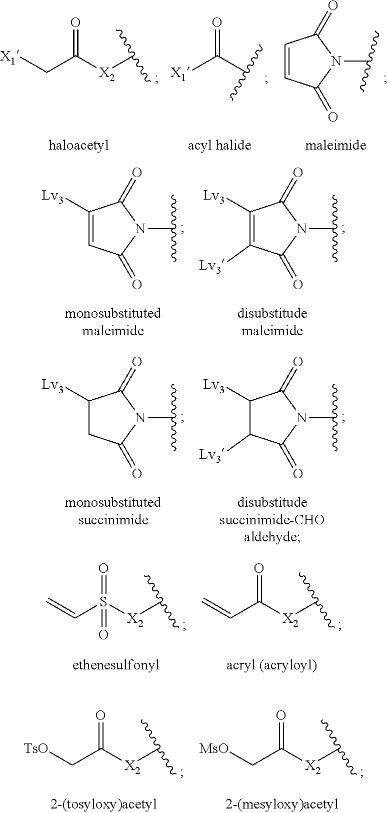 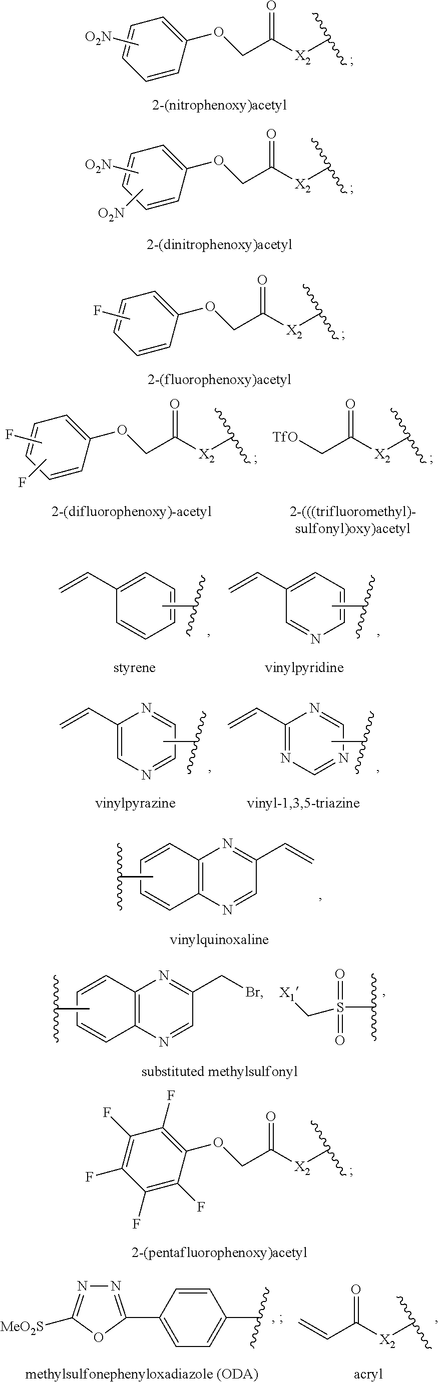 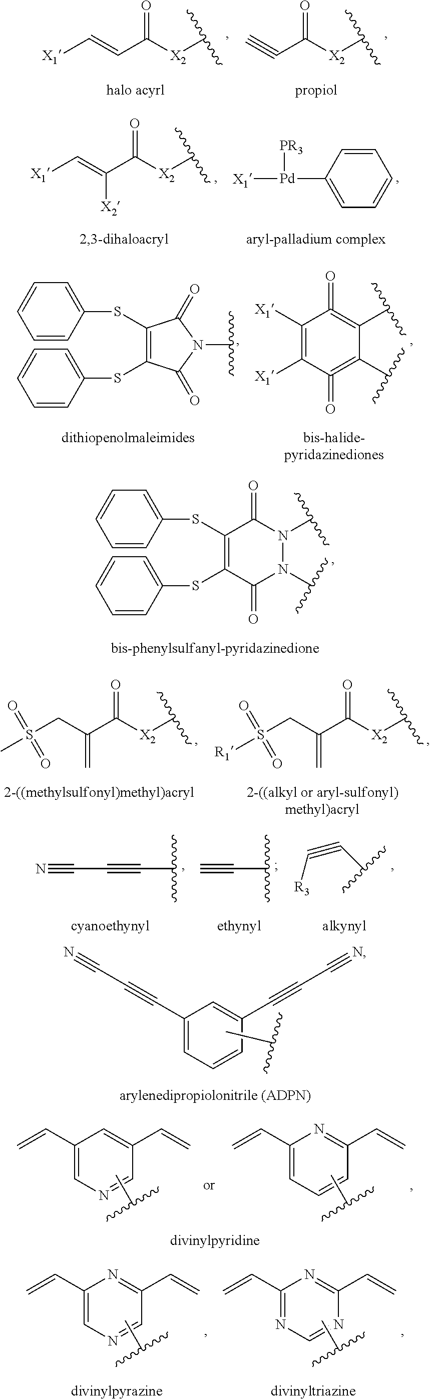 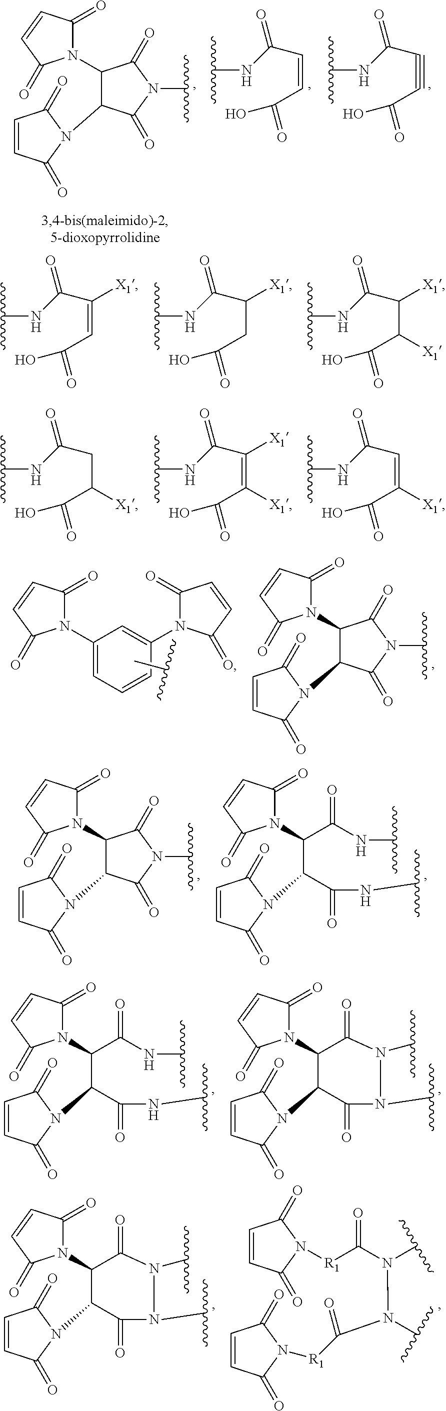 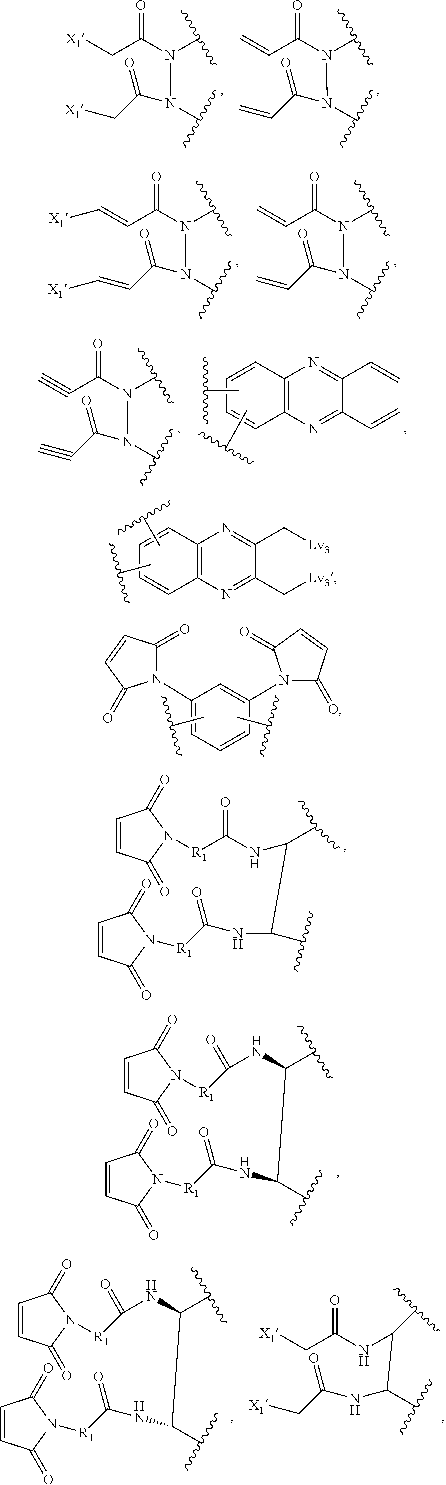 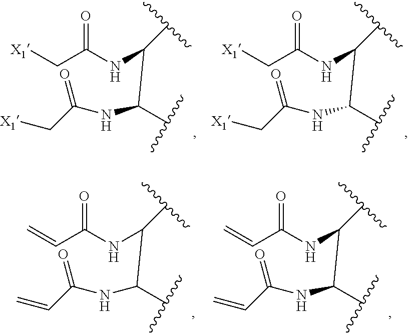 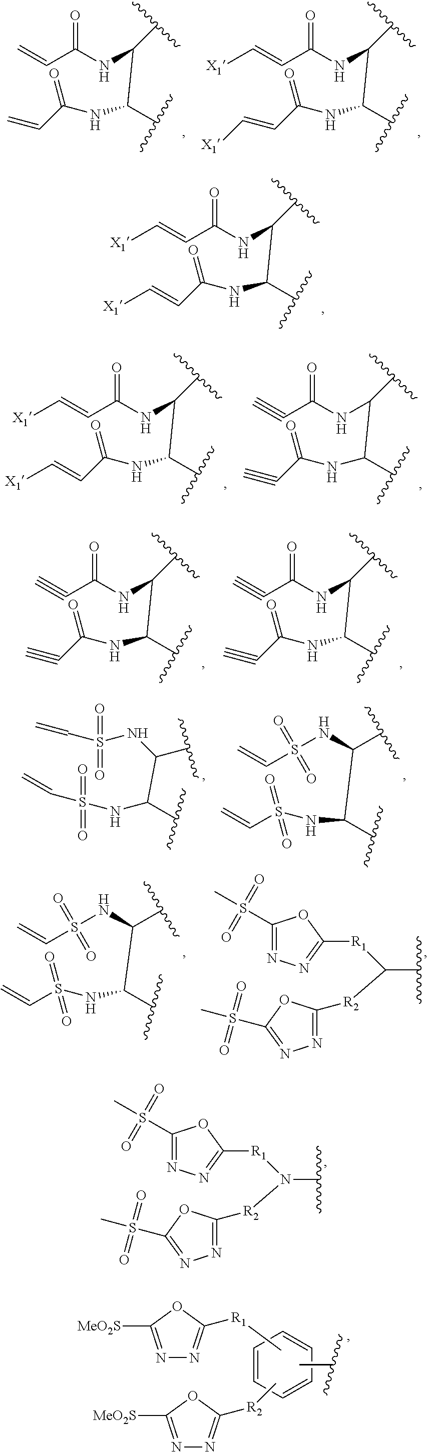 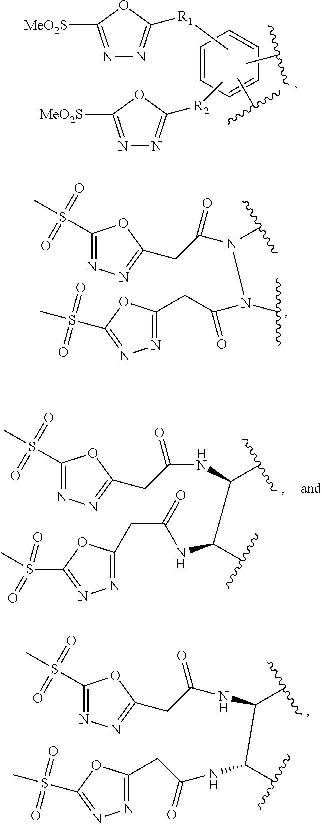
wherein X₁′ and X₂′ are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, an anhydride formed of any of the above itself, or formed with another anhydride: acetyl anhydride, formyl anhydride; or an intermediate molecule generated of any of the above with a condensation reagent for peptide coupling reactions or for Mitsunobu reactions;
wherein a condensation reaction of the formula (VIII), (IX) or (X) with the drug D₁ or/and D₂ can be conducted in a separated reaction pot;
wherein Lv₅ and Lv₆ are independently selected from: 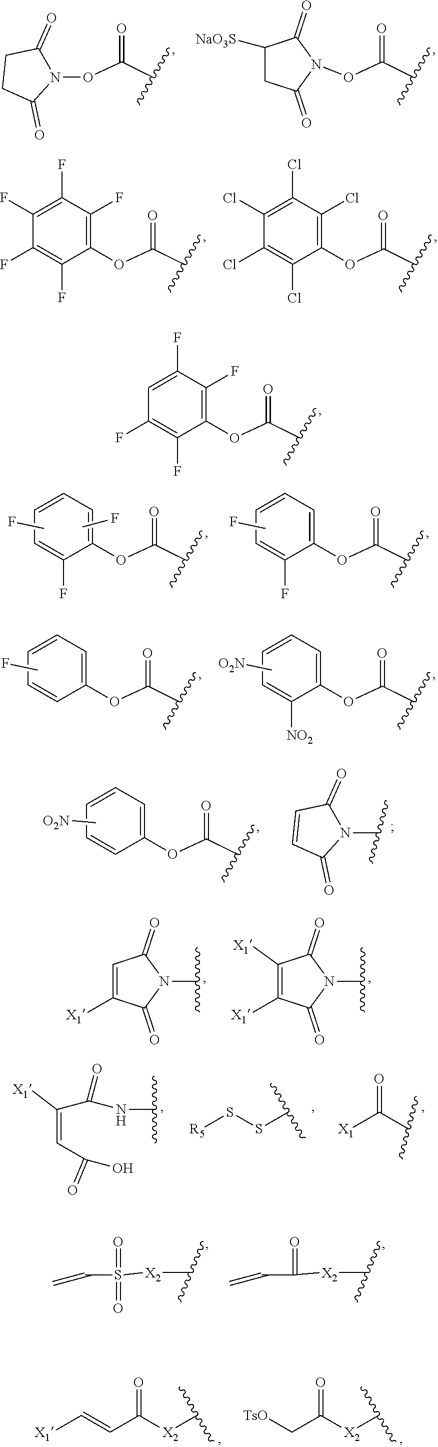 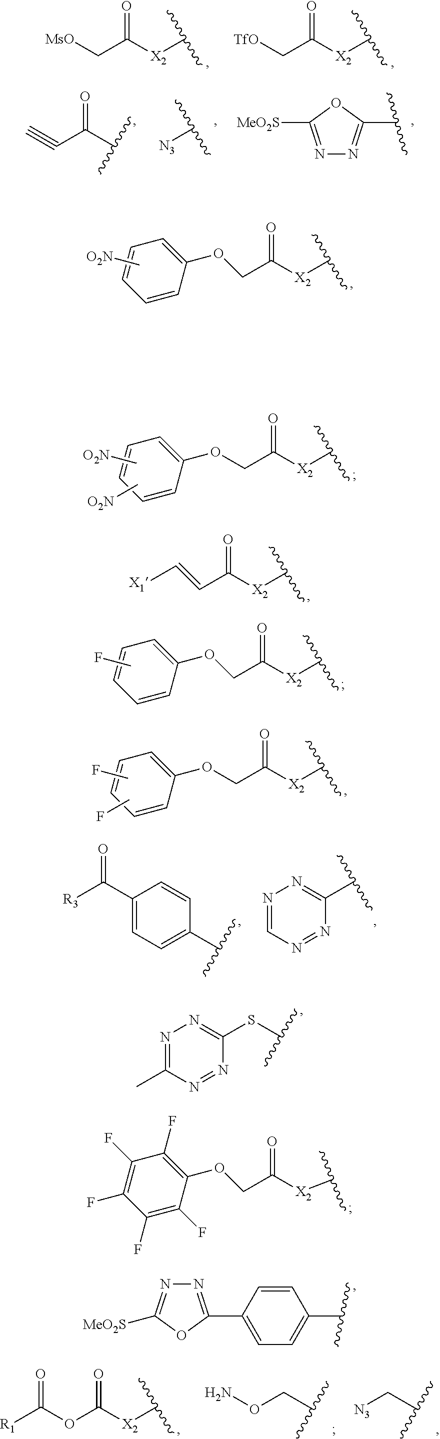 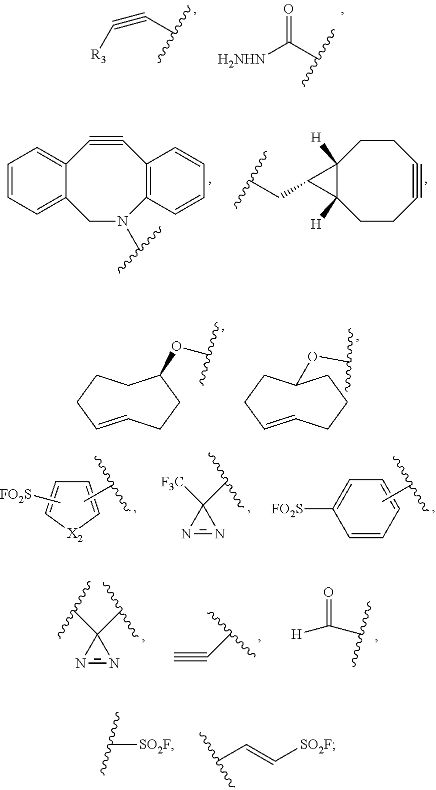
wherein X₁′ is F, Cl, Br, I, OTs (tosylate), OTf (triflate), OMs (mesylate), OC₆H₄(NO₂), OC₆H₃(NO₂)₂, OC₆F₅, OC₆HF₄, or Lv₃; X₂′ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenoxyl; N-hydroxysuccinimide (NHS); phenoxyl; benzenethiol, dinitrophenoxyl; pentafluorophenoxyl; tetrafluorophenoxyl; difluorophenoxyl; monofluorophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate, anhydride formed its self, or formed with another anhydride selected from acetyl anhydride or formyl anhydride; or an intermediate molecule generated with a condensation reagent for peptide coupling reactions or for Mitsunobu reactions; wherein Lv₅ and/or Lv₆ can be also reactive with a thiol in a cytotoxic drug as long as the reaction are at least one fold faster or slower than the reaction between Lv₁ or Lv₂ and the thiol groups in the antibody or antibody-like protein.

30. The process according to claim 29, wherein the thiol groups in the antibody or antibody-like protein generated simultaneously or sequentially react independently with formula (VIII), (IX) or (X), to form the formula (XI), (XII) or (XIII) illustrated below, followed by reaction with the drug D₁ or D₂ independently to form the conjugate of formula (V), (VI), or (VII). 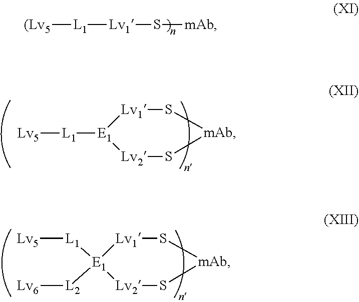
wherein Lv₅, Lv₆, L₁, L₂, E₁, Lv₁′ Lv₂′, mAb, n and n′ are the same as described in claim 29.

31. The process according to claim 28, wherein the drug D₁ and D₂ are a chromophore molecule that is capable of absorbing UV light, florescent light, IR light, near IR light, or visual light; a chromatophore molecule of a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; or a class or subclass of luciferin compounds;
the chromophore molecule is selected from, non-protein organic fluorophores: Xanthene derivatives (including fluorescein, rhodamine, Oregon green, eosin, and Texas red); Cyanine derivatives: (including cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (including dansyl and prodan derivatives); Coumarin derivatives; Oxadiazole derivatives (pyridyloxazole, nitrobenzoxadiazole and benzoxadiazole); Anthracene derivatives (anthraquinones, including DRAQ5, DRAQ7 and CyTRAK Orange); Pyrene derivatives (including cascade blue); Oxazine derivatives (including Nile red, Nile blue, cresyl violet, oxazine 170); Acridine derivatives (including proflavin, acridine orange, acridine yellow); Arylmethine derivatives (including auramine, crystal violet, malachite green); Tetrapyrrole derivatives (including porphin, phthalocyanine, bilirubin);
the chromophore molecule can be also selected from analogs and derivatives of the following fluorophore compounds: CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, IR-783, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (Biostatus, red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1, DCFH (2′,7′-Dichorodihydro-fluorescein, oxidized form), DHR (Dihydrorhodamine 123, oxidized form, light catalyzes oxidation), Fluo-3 (AM ester. pH >6), Fluo-4 (AM ester. pH 7.2), Indo-1 (AM ester, low/high calcium (Ca2+)), and SNARF (pH 6/9).

32. The process according to claim 27, wherein the drug D₁ and D2 are independently a polyalkylene glycol selected from poly(ethylene glycols) (PEGs), poly(propylene glycol) and copolymers of ethylene oxide and propylene oxide; the polyalkylene glycol has a molecular weight of from about 10 Daltons to about 200 kDa.

33. The process according to claim 28, wherein the drug D₁ and D2 are selected from a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (including dimers of pyrrolobenzodiazepine (PBD), tomaymycin, anthramycin, indolinobenzodiazepines, imidazobenzothiadiazepines, or oxazolidinobenzo-diazepines) and their analogs, a calicheamicin or an enediyne antibiotic and its analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin and its analogs, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT) inhibitor, an immunotoxin, a cell receptor agonist, a cell stimulating molecule or intracellular signalling molecule, one, two or more DNA, RNA, mRNA, small interfering RNA (siRNA), microRNA (miRNA), and PIWI interacting RNAs (piRNA), and stereoisomers, isosteres, analogs, or derivatives thereof;
wherein:
(a) Tubulysin and its analogs have the following formula (IV): 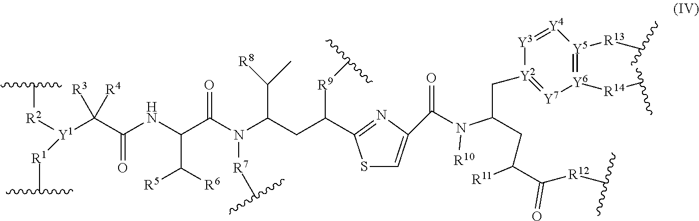
or a derivative with one or more isotopes; or a pharmaceutically acceptable salt, hydrate, or hydrated salt thereof, or a polymorphic crystalline structure thereof, or an optical isomer, racemate, diastereomer or enantiomer thereof,
wherein [symbol] is a linkage site that links to L₁ and/or L₂ independently;
wherein R¹, R², R³, and R⁴ are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl; or R¹ and R², R¹ and R³, R² and R³, R³ and R⁴, R⁵ and R⁶, R¹¹ and R¹² or R¹³ and R¹⁴ form a 3-7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R′, R¹⁰ and R¹¹ are independently H, or C₁-C₄ alkyl or heteroalkyl;
wherein R⁷ is independently H, R¹⁴, —R¹⁴C(═O)X¹R¹⁵; or —R¹⁴X₁R¹⁵; X¹ is O, S, S—S, NH, CH₂ or NR¹⁴;
wherein R⁹ is H, OH, —O—, ═O, —OR¹⁴, —OC(═O)R¹⁴, —OC(═O)NHR¹⁴—, —OC(═O) R¹⁴SSR¹⁵—, OP(═O)(OR¹⁴)—, —OC(═O)NR¹⁴R¹⁵, OP(═O)(OR¹⁴), or OR¹⁴OP(═O)(OR¹⁵);
wherein R¹¹ is independently H, R¹, —R¹⁴C(═O)R¹⁶, —R¹X₂R², —R¹C(═O)X², wherein X² is —O—, —S—, —NH—, —N(R²)—, —O—R¹—, —S—R¹—, —S(═O)—R′—, or —NR²;
wherein R¹² is R¹, —OH, —SH, —NH₂, NH, NHNH₂, —NH(R¹⁵), —OR¹⁵, —R¹⁵COR¹⁶, —R¹⁵COOR¹⁶, —R¹⁵C(O)NH₂, —R¹⁵C(O)NHR¹⁷, —SR¹⁶, R¹⁵S(═O)R¹⁶, —R¹⁵P(═O)(OR¹⁷)₂, —R¹⁵OP(═O)(OR¹⁷)₂, —CH₂OP(═O)(OR¹⁷)₂, —R¹⁵SO₂R¹⁷, —R¹⁵X₂R¹⁶, —R¹⁵C(═O)X², where X² is —O—, OH, SH, —S—, NH₂, —NH—, —N(R¹⁵)—, —O—R¹⁵—, —S—R¹⁵—, —S(═O)—R¹⁵—, CH₂ or —NHR¹⁵—R¹³ and R¹⁴ are independently H, O, S, NH, N(R¹⁵), NHNH, —OH, —SH, —NH₂, NH, NHNH₂, —NH(R¹⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N—(C₄-C₁₂ glycoside); C₁-C₈ alkyl, or heteroalkyl; C₂-C₈ alkenyl, alkynyl, heteroalkyl, or heterocycloalkyl; C₃-C₈ aryl, Ar-alkyl, carbocyclic, cycloalkyl, heteroalkylcycloalkyl, alkylcarbonyl, or heteroaryl, or C₂-C₈ ester, ether, or amide; or a peptide containing 1-8 amino acids (NH(Aa)₁₋₈ or CO(Aa)₁₋₈ (N-terminal or C-terminal (Aa)r, r=1-8 of the same or different sequences of amino acids), or polyethyleneoxy unit of formula (OCH₂CH₂)ₚ or (OCH₂CH(CH₃))ₚ, wherein p is an integer from 0 to about 1000, or a combination of two or more of the above groups; X² is O, S, S—S, NH, CH₂, OH, SH, NH₂, CHR¹ or NR¹;
R¹⁵, R¹⁶ and R¹⁷ is independently H, C₁-C₈ alkyl, or heteroalkyl; C₂-C₈ alkenyl, alkynyl, heteroalkyl, or heterocycloalkyl; C₃-C₈ aryl, Ar-alkyl, carbocyclic, cycloalkyl, heteroalkylcycloalkyl, alkylcarbonyl, heteroaryl, or alkylcarbonyl, or Na⁺, K⁺, Cs⁺, Li⁺, Ca²⁺, Mg⁺, Zn²⁺, N⁺(R¹)(R²)(R³)(R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N(R₁), thus Y², Y⁴ Y⁵ Y⁶ and Y⁷ form a heteroaromatic ring of furan, pyrrole thiophene, thiazole, oxazole and imidazole, pyrazole, triazole, tetrazole, or thiadiazole; when q=1, Y³, Y⁴, Y⁵, Y⁶ and Y⁷ are independently CH or N, thus Y², Y³ Y⁴ Y⁵ Y⁶ and Y⁷ form an aromatic ring of benzene, pyridine, pyridazine, pyrimidine, pyrazine, triazine, tetrazine, or pentazine;
the tubulysin analogs have the structures shown below: 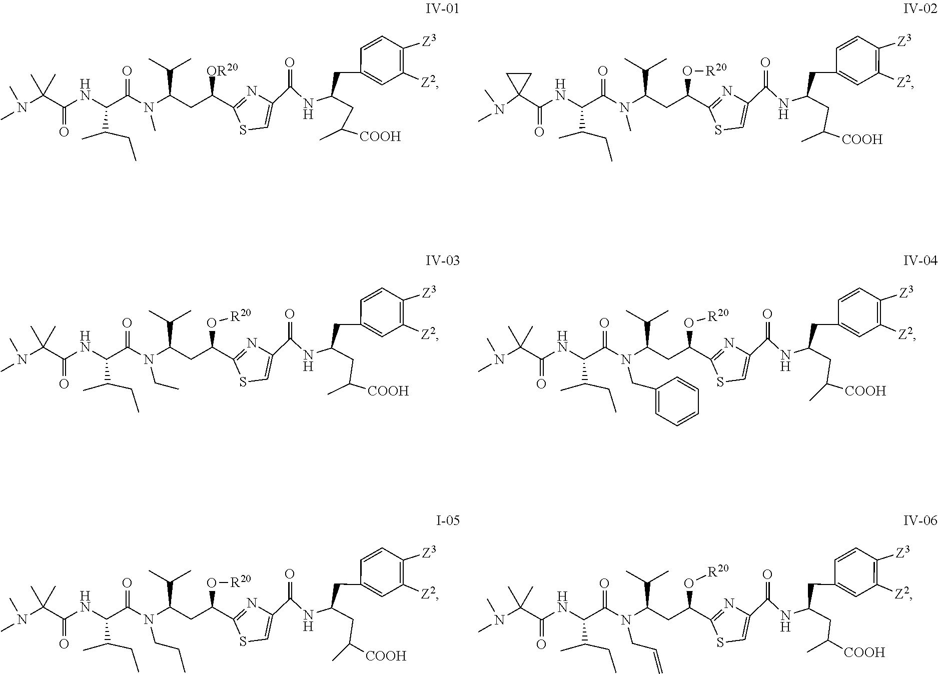 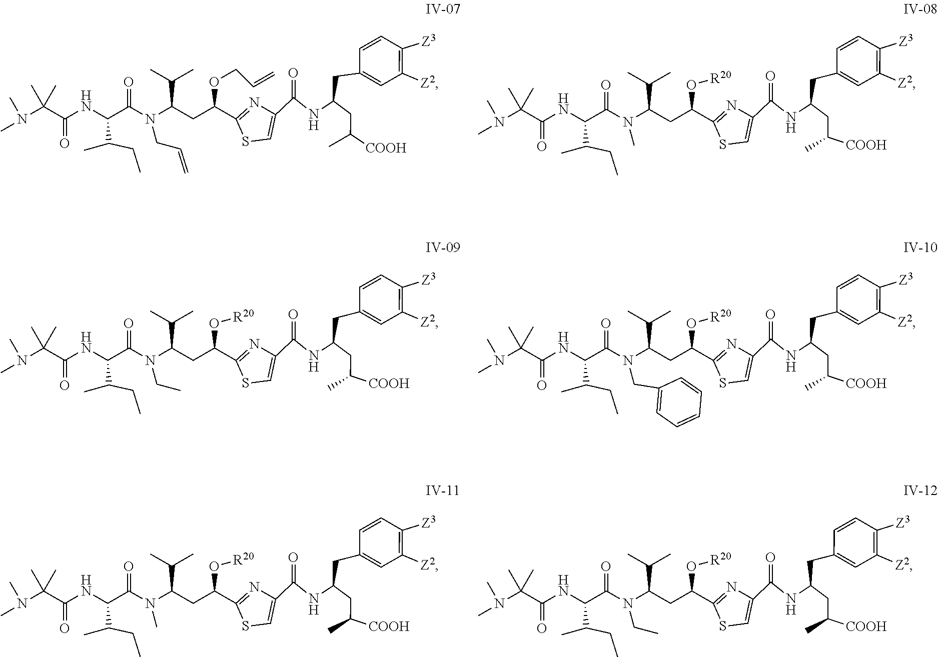 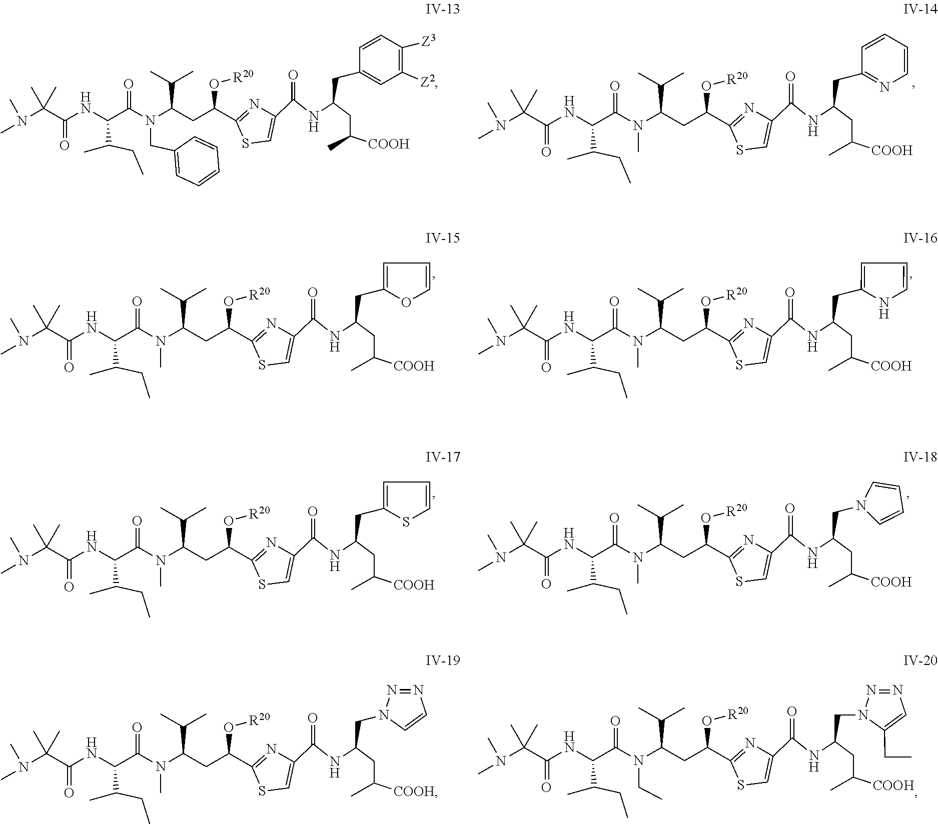 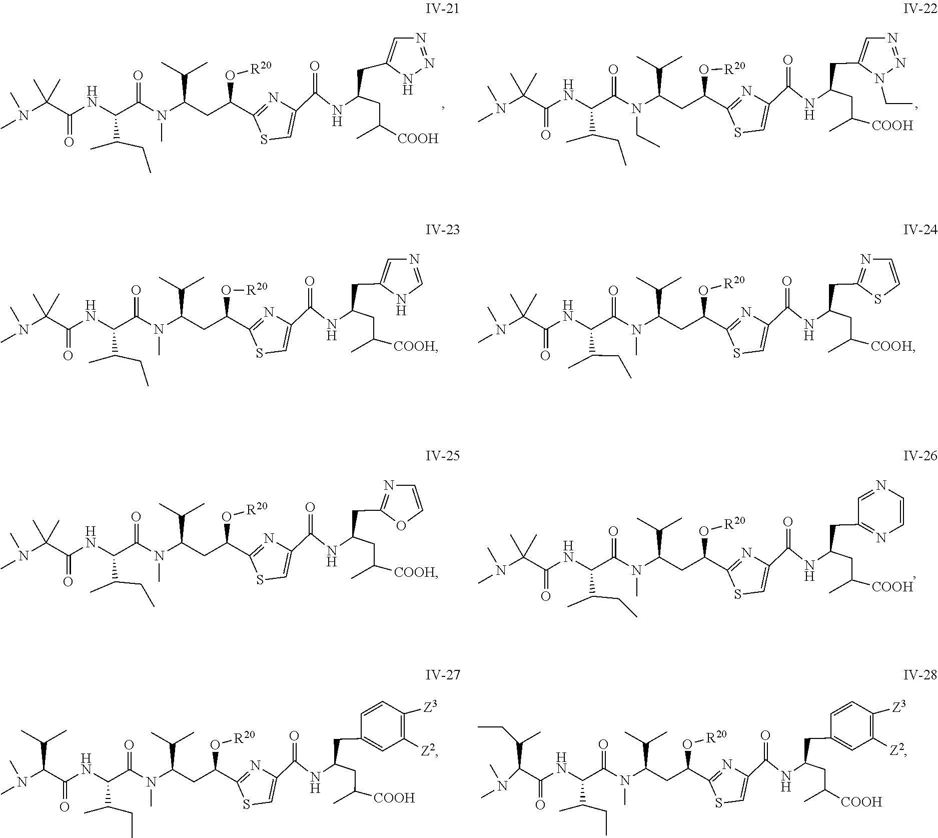 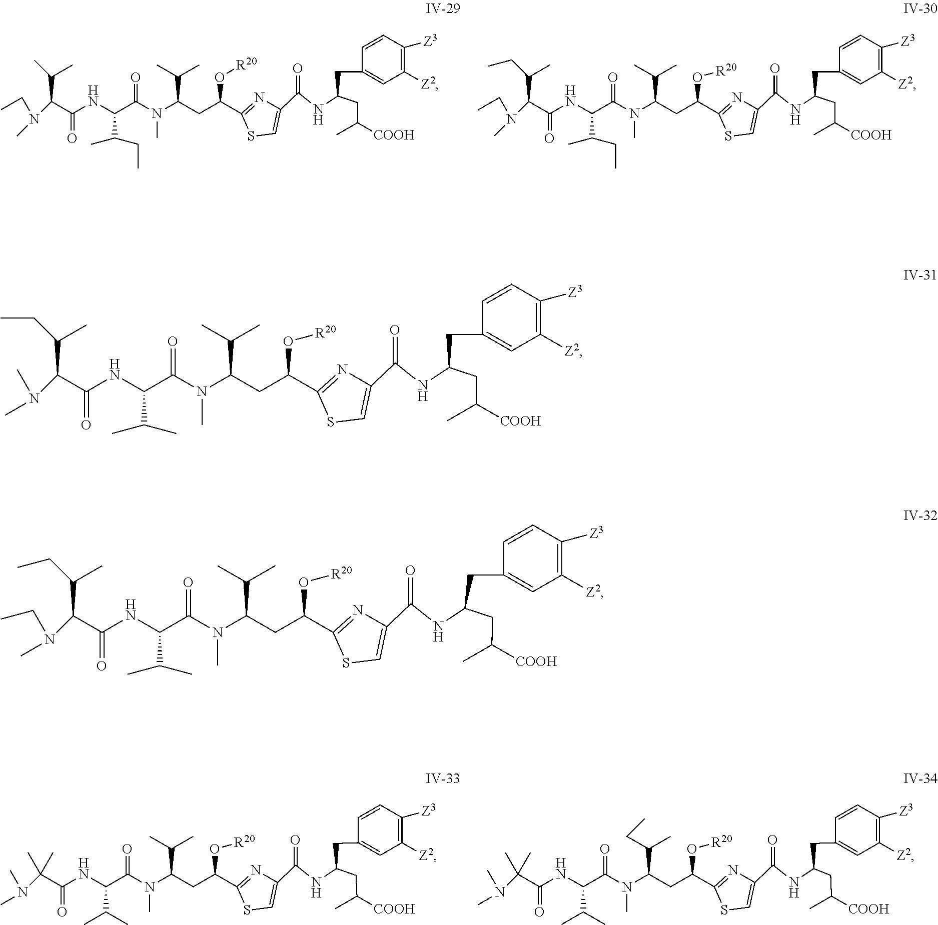 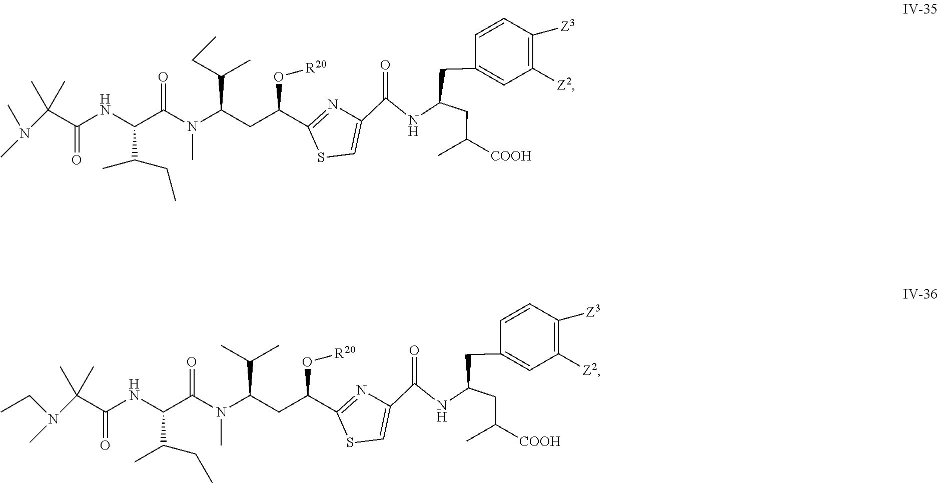 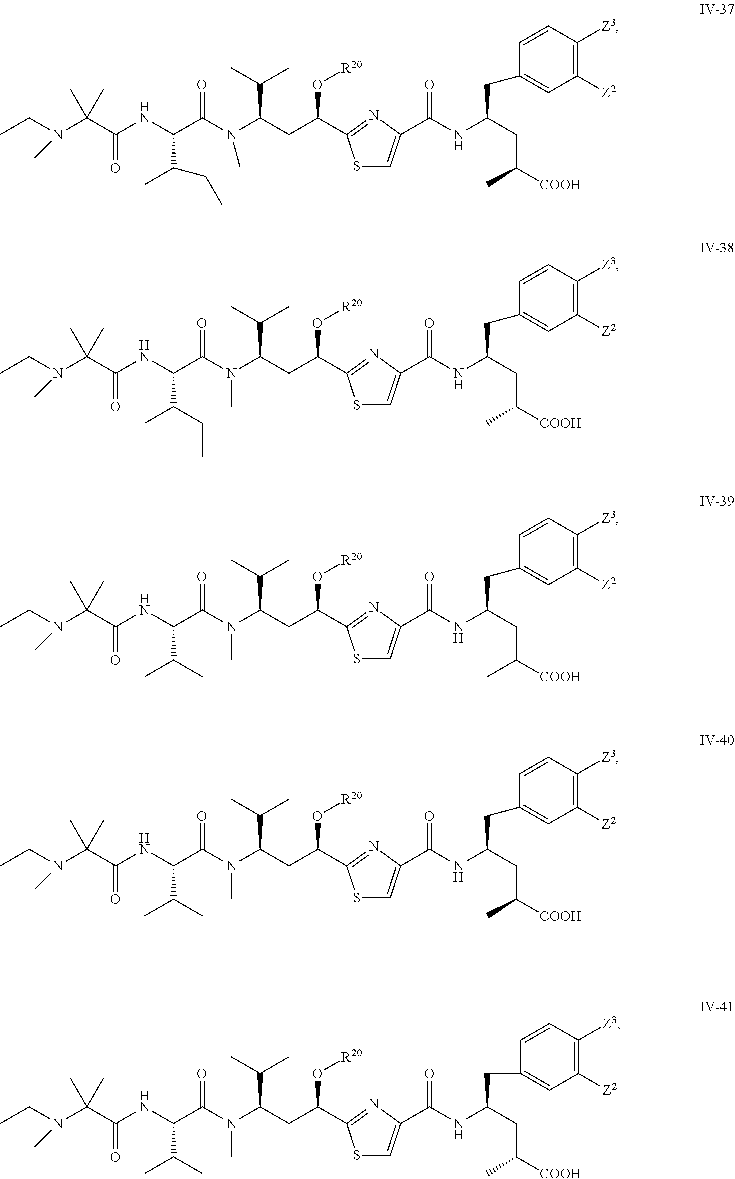 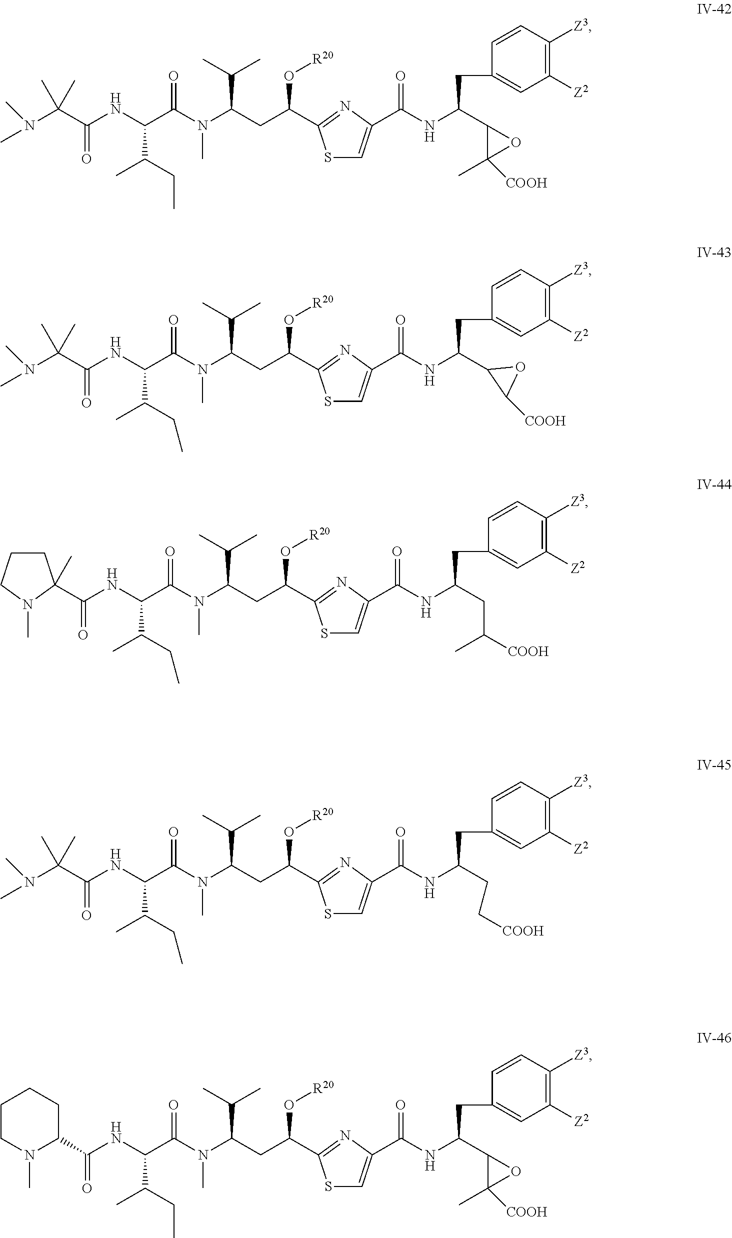 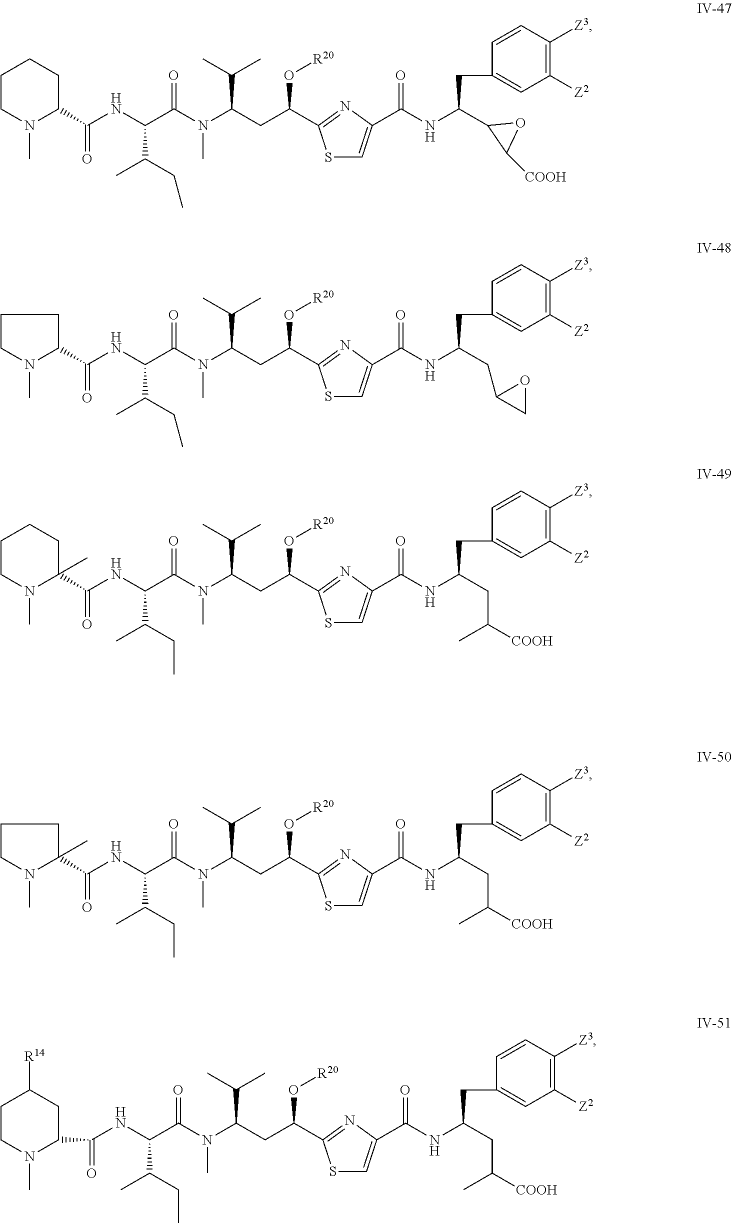 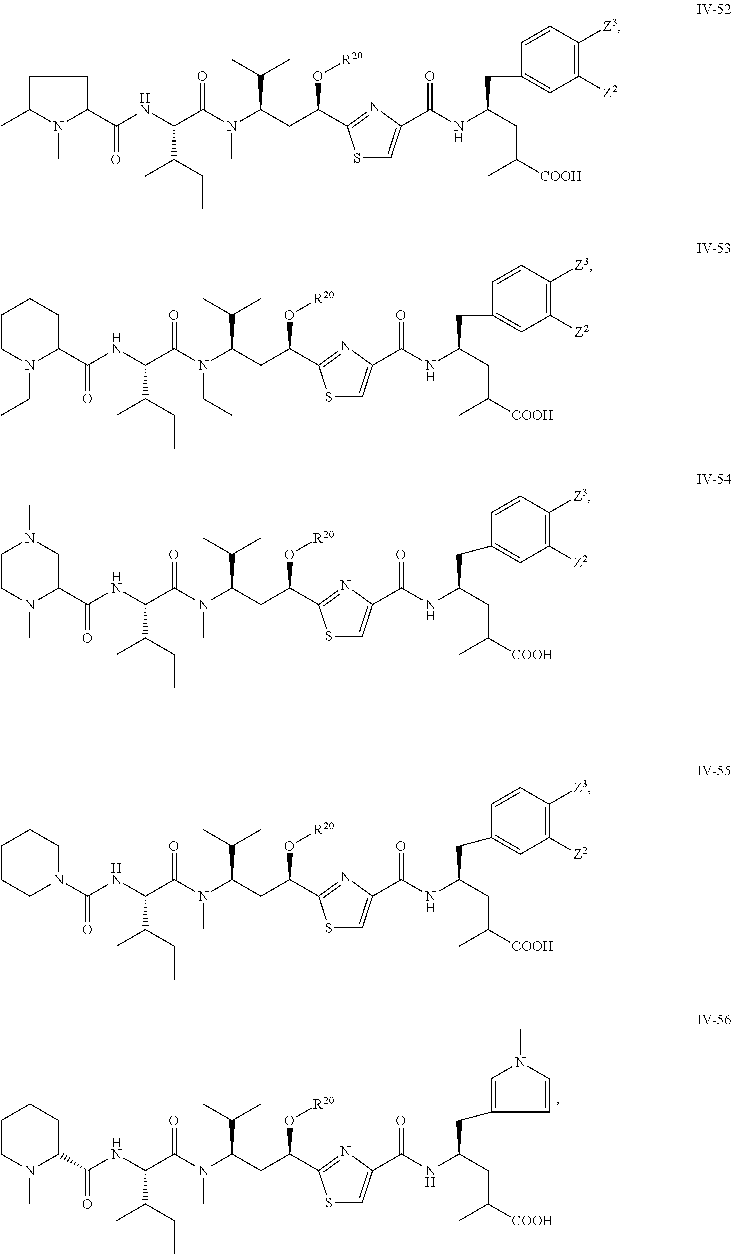 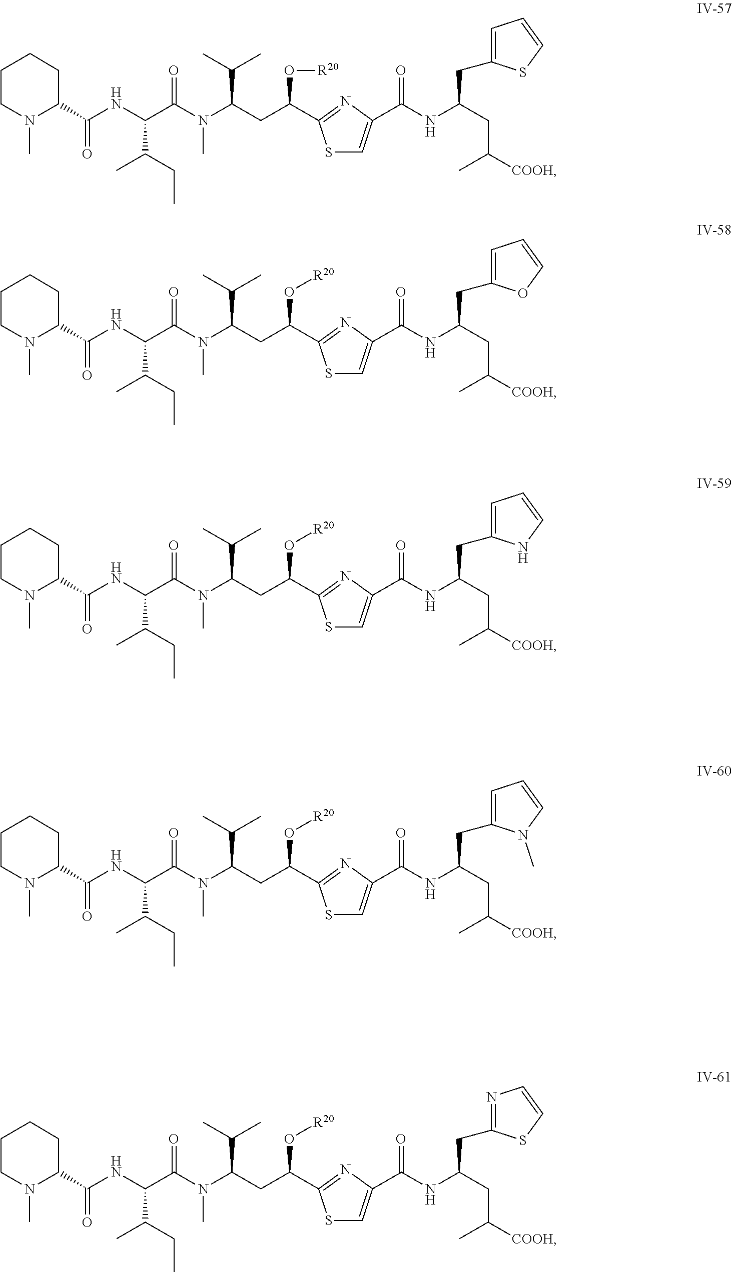 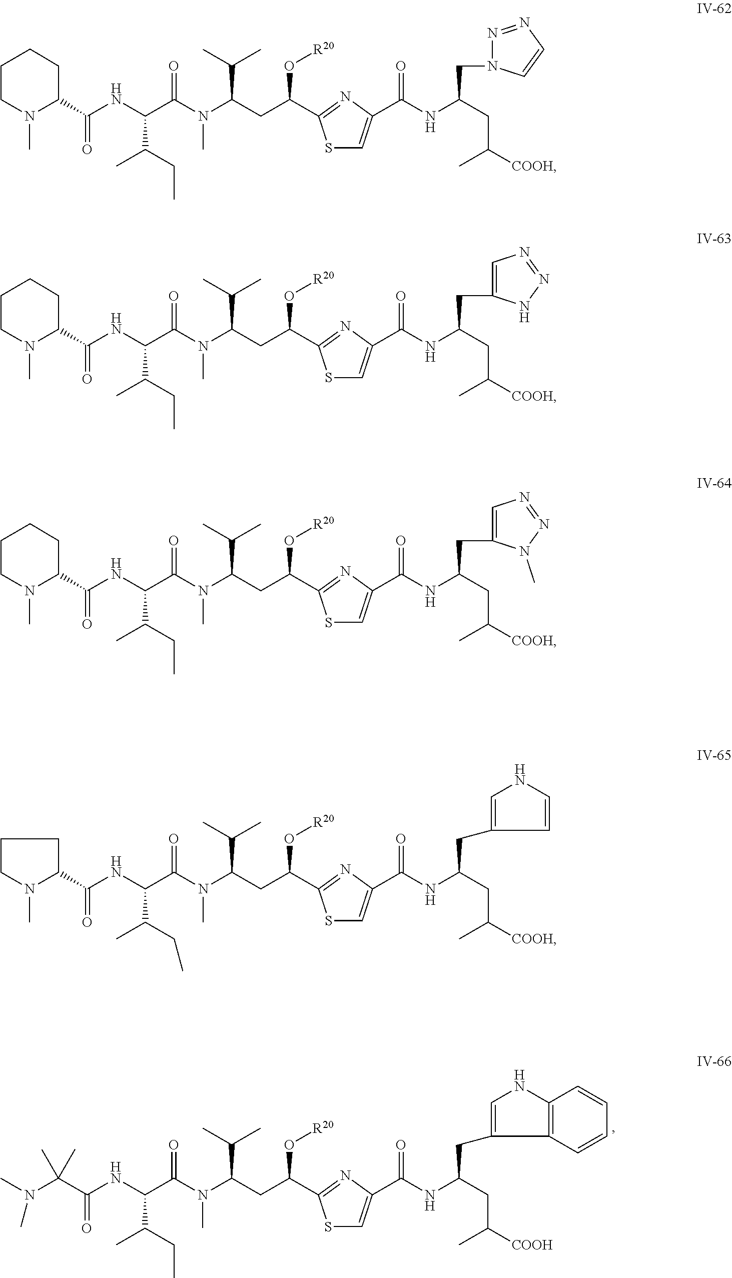 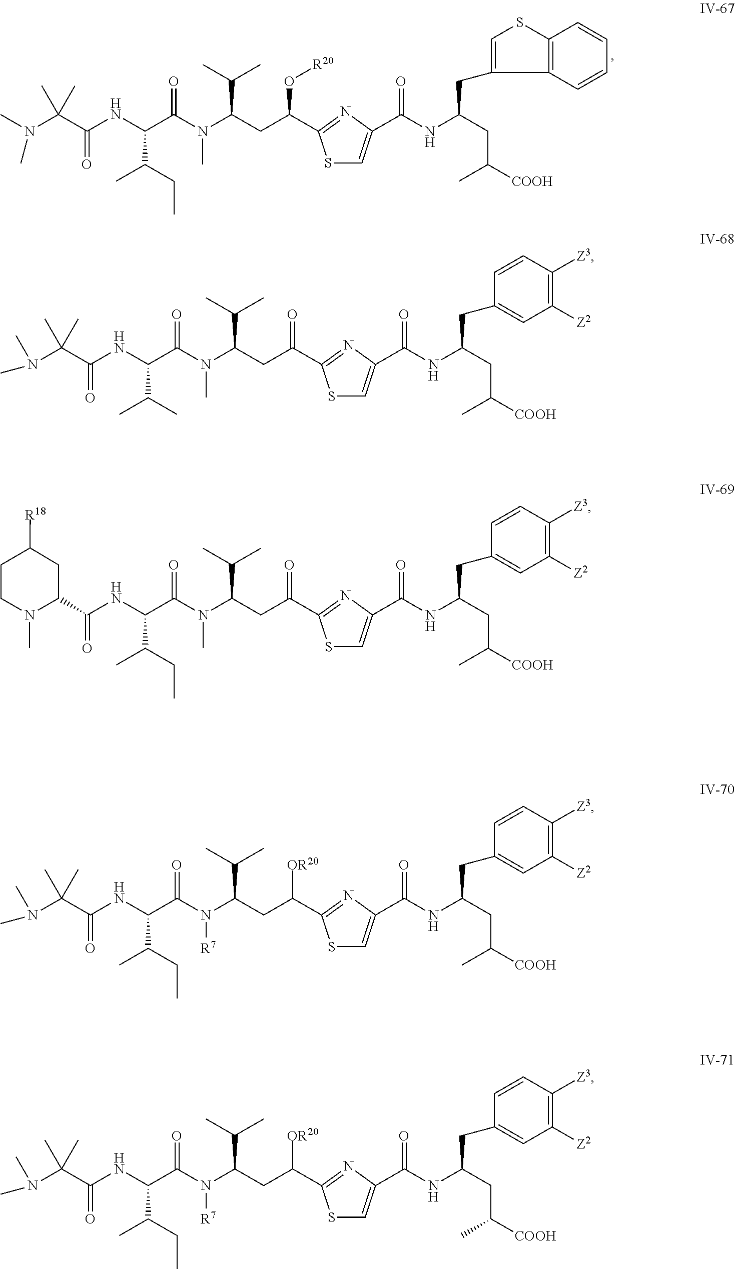 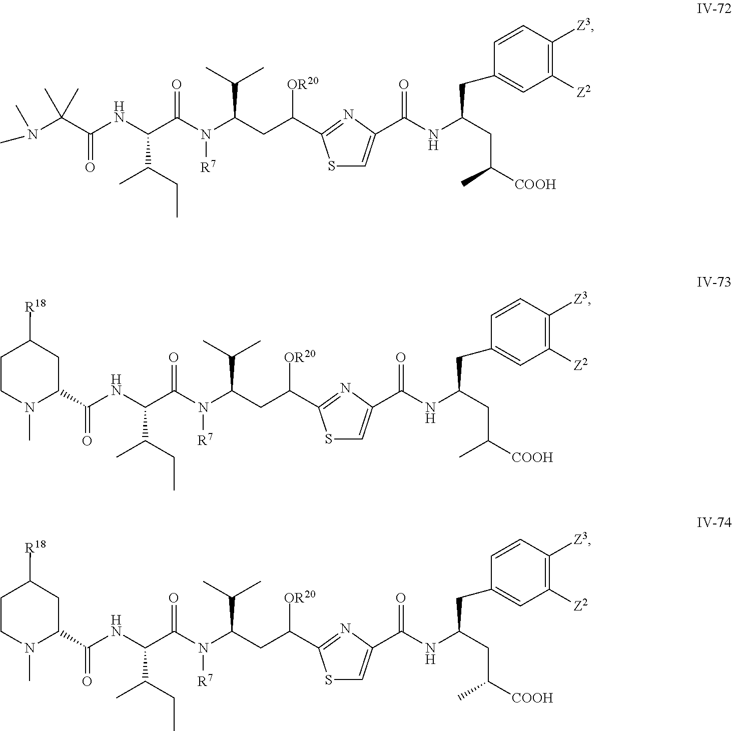 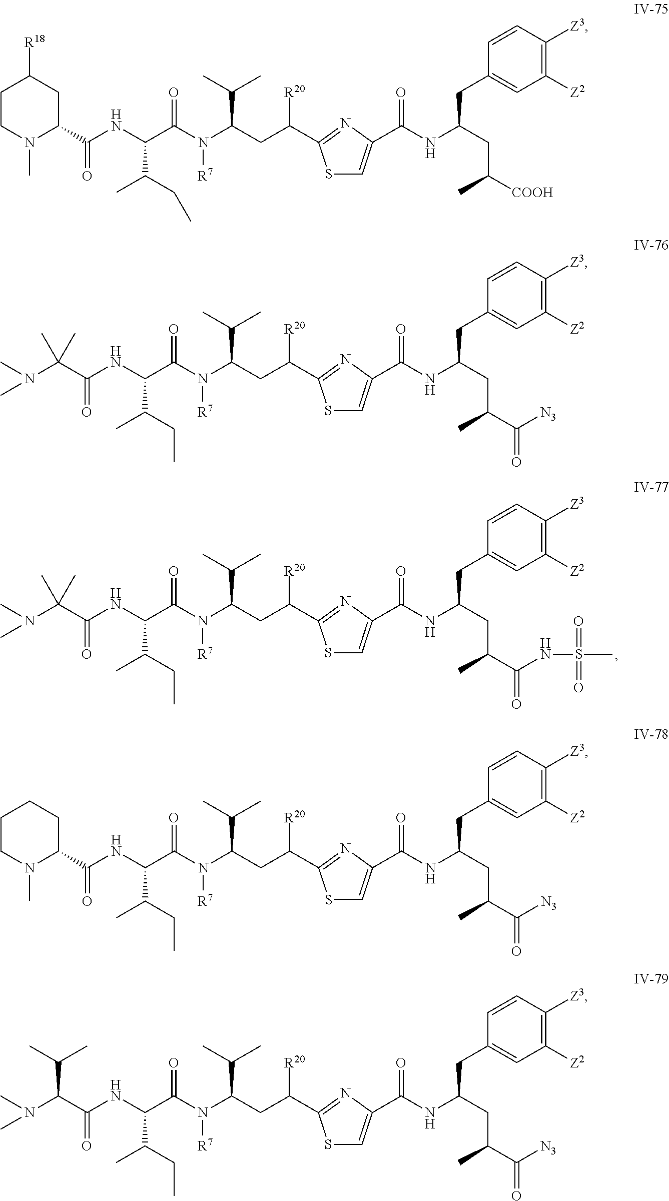
wherein R²⁰ is H; C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); or C₁-C₈ carboxylate, ester, ether, or amide; or 1-8 amino acids; or polyethyleneoxy unit of formula (OCH₂CH₂)ₚ or (OCH₂CH(CH₃))ₚ, wherein p is an integer from 0 to about 1000; or R²⁰ is absent and the oxygen forms a ketone, or a combination of two or more of the above groups;
Z² and Z³ are independently H, OH, NH₂, O, NH, COOH, COO, C(O), C(O), C(O)NH, C(O)NH₂, R¹⁸, OCH₂OP(O)(OR¹⁸)₂, OC(O)OP(O)(OR¹⁸)₂, OPO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); R¹⁷ and R¹⁸ are independently H, C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸);
R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁-C₈ alkyl or carboylate; C₂-C₈ alkenyl, alkynyl, alkylcycloalkyl, or heterocycloalkyl; C₃-C₈ aryl or alkylcarbonyl; or a pharmaceutical salt thereof,
X is O, S, NH, NHNH, or CH₂;
R⁷ is the same as defined above; wherein linkage sites, “ [symbol] ” in formula (IV-01)-(IV-79) have the same linkage sites according to formula (IV);
(b) the calicheamicins and their related enediyne antibiotics have the following formula: 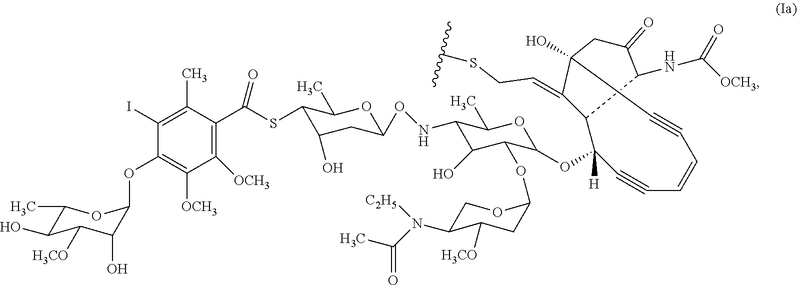 [SMILES: *SC/C=C1\C2[C@@H](OC3OC(C)C(NOC4CC(O)C(SC(=O)c5c(C)c(I)c(OC6OC(C)C(O)C(OC)C6O)c(OC)c5OC)C(C)O4)C(O)C3OC3CC(OC)C(N(CC)C(C)=O)CO3)C#C/C=C\C#C[C@]1(O)CC(=O)[C@@H]2NC(=O)OC]
or a derivative with one or more isotopes, or a pharmaceutically acceptable salt, hydrate, or hydrated salt thereof, or a polymorphic crystalline structure thereof, or an optical isomer, racemate, diastereomer or enantiomer thereof,
wherein [symbol] is a site linked to L₁ or L₂;
(c) the Maytansines or their derivatives maytansinoids have the following formula: 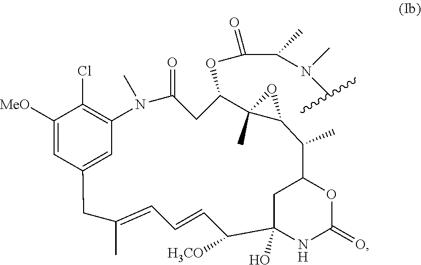
wherein [symbol] is a site linked to L₁ or L₂;
(d) the camptothecin (CPTs) and its derivatives have the following formula: 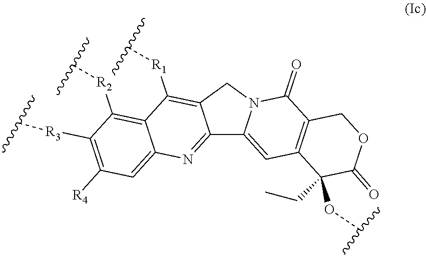
or a derivative with one or more isotopes, or a pharmaceutically acceptable salt, hydrate, or hydrated salt thereof; or a polymorphic crystalline structure thereof; or an optical isomer, racemate, diastereomer or enantiomer thereof;
wherein R₁, R₂ and R₄ are independently selected from H, F, Cl, Br, CN, NO₂, C₁-C₈ alkyl; O—C₁-C₈ alkyl; NH—C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, amide, carbonate, urea, or carbamate;
R₃ is H, OH, NH₂, C₁-C₈ alkyl; O—C₁-C₈ alkyl; NH—C₁-C₈ alkyl; C₂-C₈ heteroalkyl, alkylcycloalkyl, or heterocycloalkyl; or C₂-C₈ ester, ether, amide, carbonate, urea, or carbamate; or R₁ and R₂, R₂ and R₃ and R₃ and R₄ independently form a 5-7 membered carbocyclic, heterocyclic, heterocycloalkyl, aromatic or heteroaromatic ring system; “ [symbol] ” is a site in the molecule that can be linked to L₁ or L₂;
the camptothecin (CPTs) and its derivatives have the following formula: 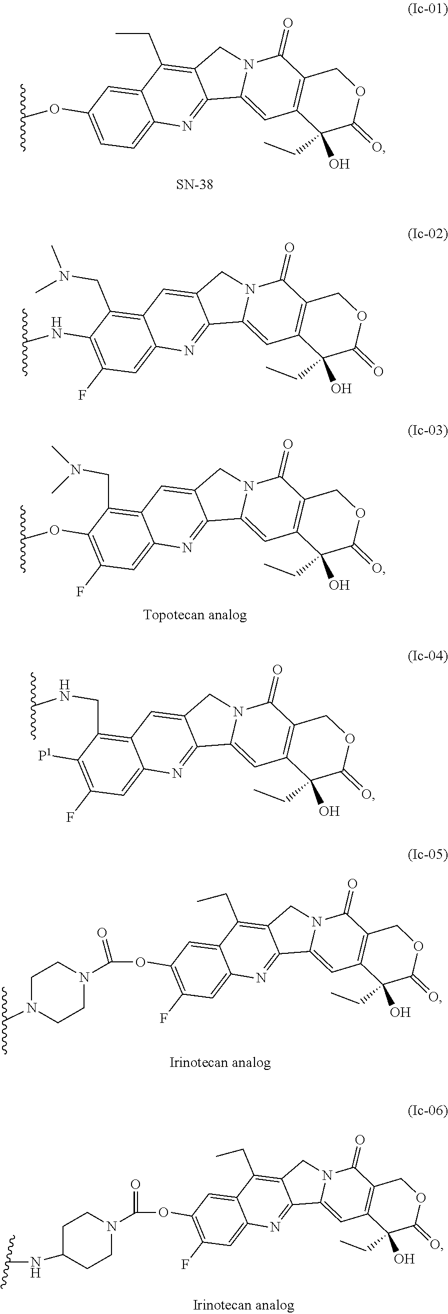 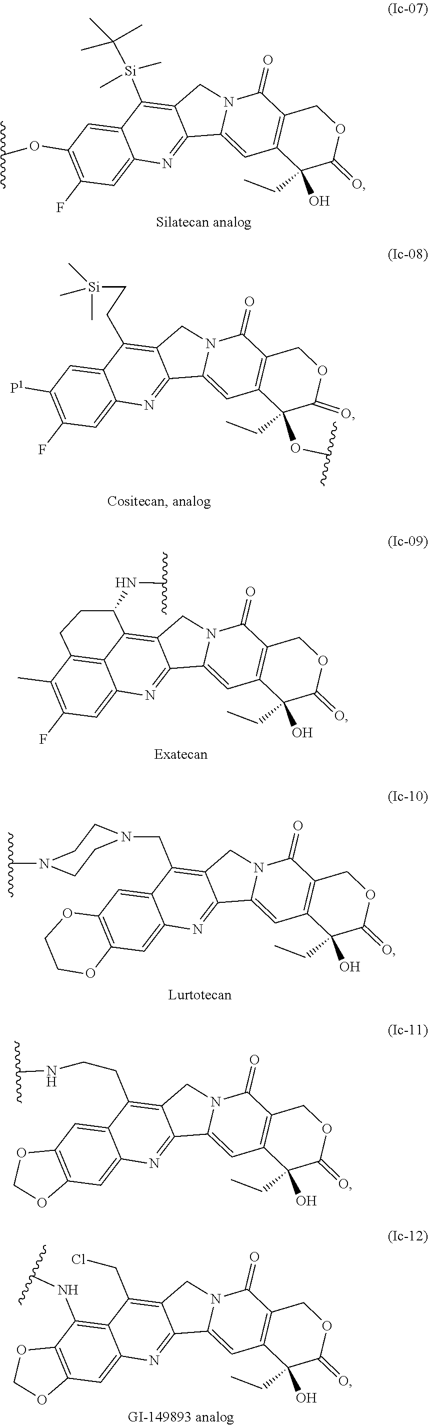 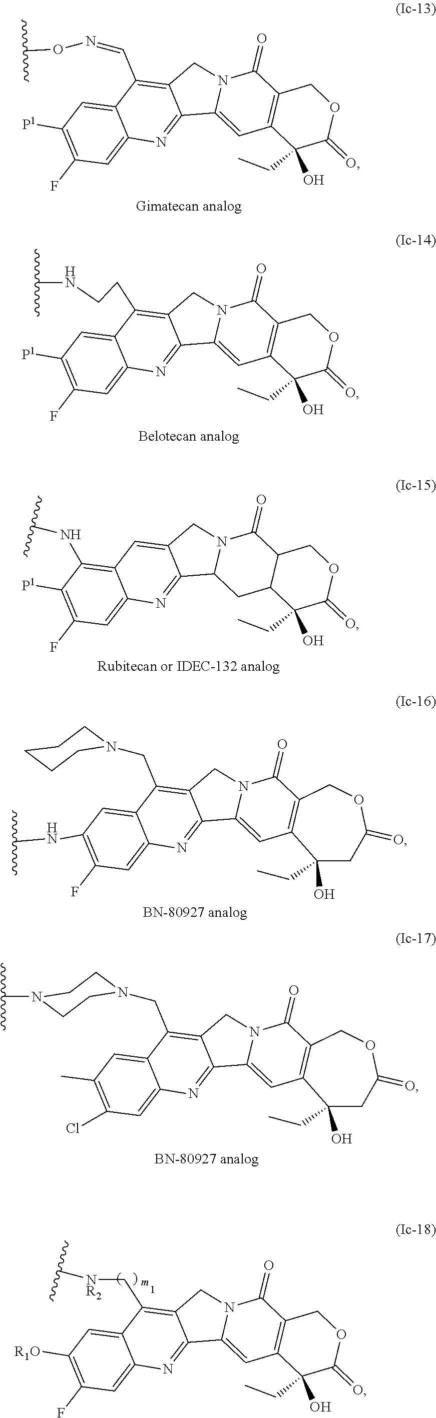
or a derivative with one or more isotopes, or a pharmaceutically acceptable salt, hydrate, or hydrated salt thereof; or a polymorphic crystalline structure thereof, or an optical isomer, racemate, diastereomer or enantiomer thereof,
wherein [symbol] is a site linked to L₁ or L₂; P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ linear or branched alkyl), C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); R¹⁷ and R¹⁸ are independently H, C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); R₁ and R₂ are independently H; O—C₁-C₈ alkyl; C₂-C₈ of heteroalkyl, alkylcycloalkyl, heterocycloalkyl; C₃-C₈ of aryl, or Ar-alkyl;
(e) the Combretastatins have the following formula: 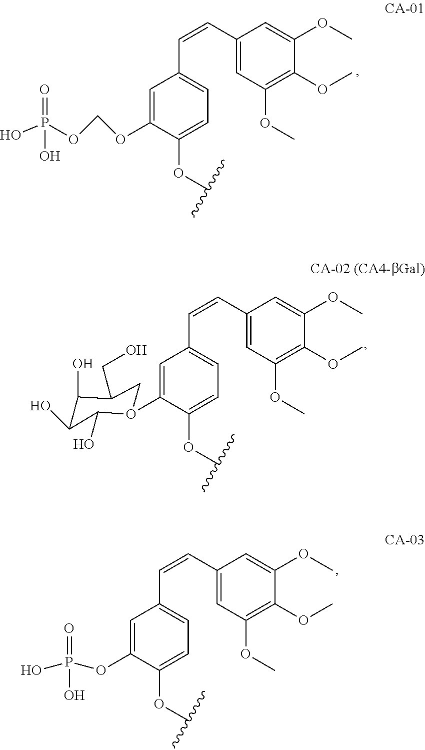 [symbol]
is a site in the molecule that can be linked to L₁ or L₂;
(f) the Taxanes have the following formula: 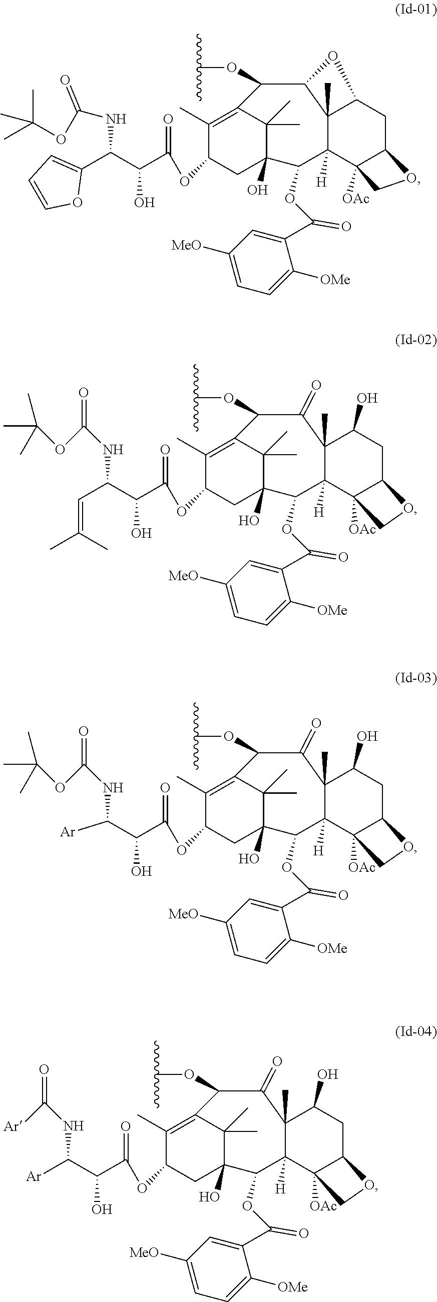
wherein [symbol] is a site linked to L₁ or L₂; Ar and Ar′ are independently aryl or heteroaryl;
(g) the anthracyclines have the following formula: 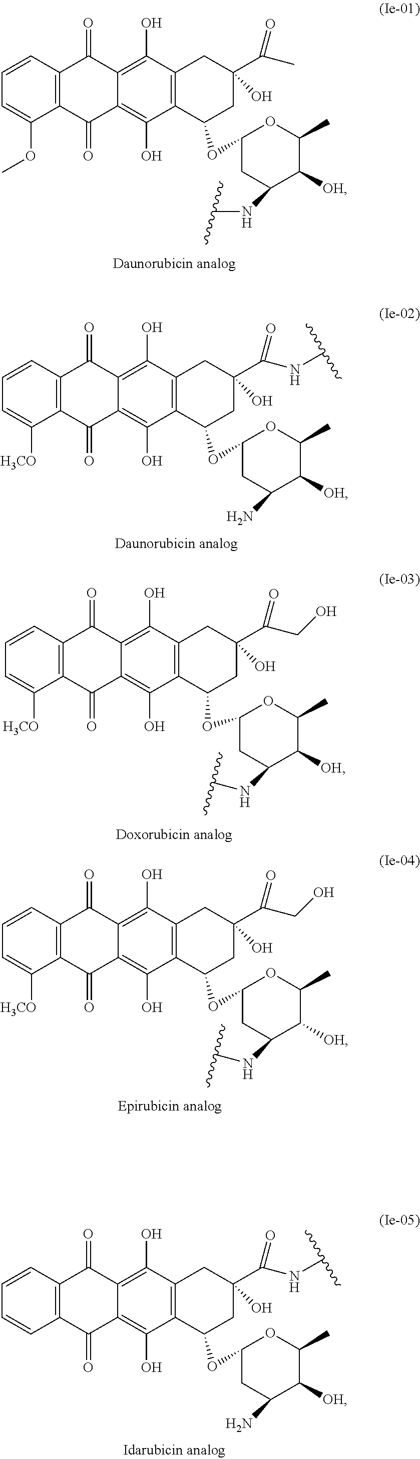 [SMILES: *N[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O ; *NC(=O)[C@]1(O)Cc2c(O)c3c(c(O)c2[C@@H](O[C@H]2C[C@H](N)[C@H](O)[C@H](C)O2)C1)C(=O)c1c(OC)cccc1C3=O ; *N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O ; *N[C@H]1C[C@H](O[C@H]2C[C@](O)(C(=O)CO)Cc3c(O)c4c(c(O)c32)C(=O)c2c(OC)cccc2C4=O)O[C@@H](C)[C@@H]1O ; *NC(=O)[C@]1(O)Cc2c(O)c3c(c(O)c2[C@@H](O[C@H]2C[C@H](N)[C@H](O)[C@H](C)O2)C1)C(=O)c1ccccc1C3=O] 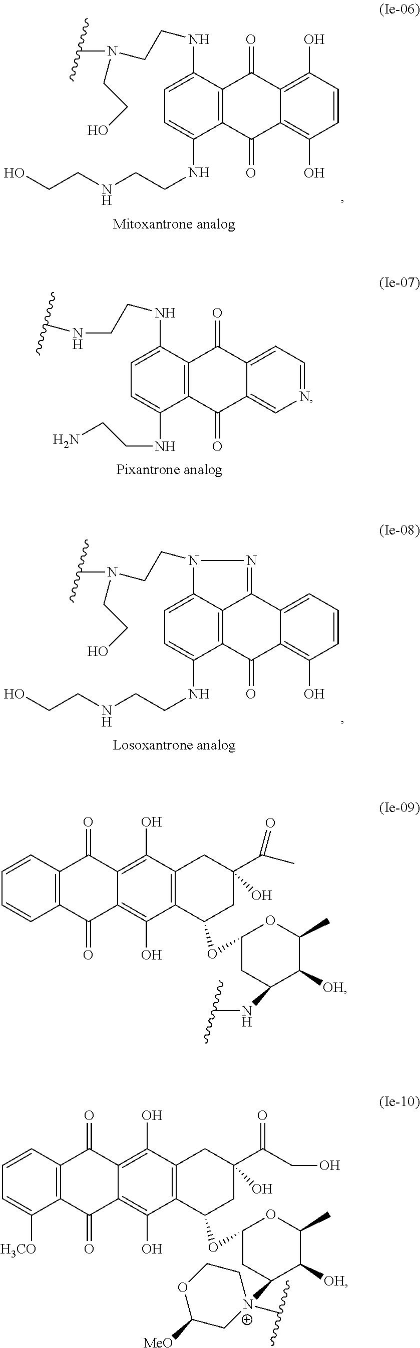 [SMILES: *N(CCO)CCNc1ccc(NCCNCCO)c2c1C(=O)c1c(O)ccc(O)c1C2=O ; *NCCNc1ccc(NCCN)c2c1C(=O)c1ccncc1C2=O ; *N(CCO)CCn1nc2c3c(c(NCCNCCO)ccc31)C(=O)c1c(O)cccc1-2 ; *N[C@H]1C[C@H](O[C@H]2C[C@](O)(C(C)=O)Cc3c(O)c4c(c(O)c32)C(=O)c2ccccc2C4=O)O[C@@H](C)[C@H]1O ; *[N+]1([C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)CCO[C@H](OC)C1] 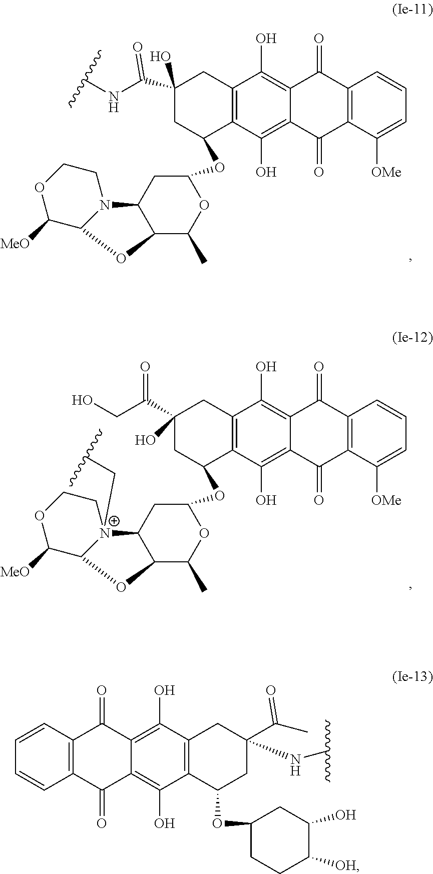
Amrubicin analog,
wherein [symbol] is a site that links to L₁ or L₂;
(h) the Vinca alkaloids are selected from vinblastine, vincristine, vindesine, leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine and have the following formula: 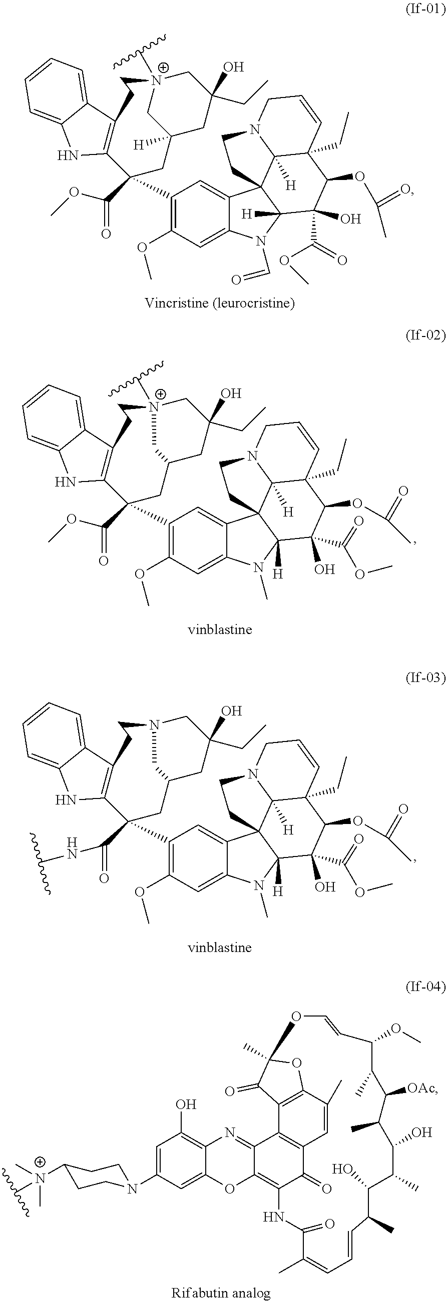 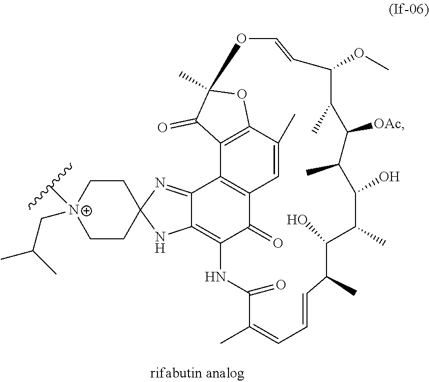
wherein [symbol] is a site linked to L₁ or L₂;
(i) the Dolastatins and their peptidic analogs and derivatives are selected from dolastatin 10, auristatin E (AE), auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), Auristatin F phenylene diamine (AFP) and have the following formula: 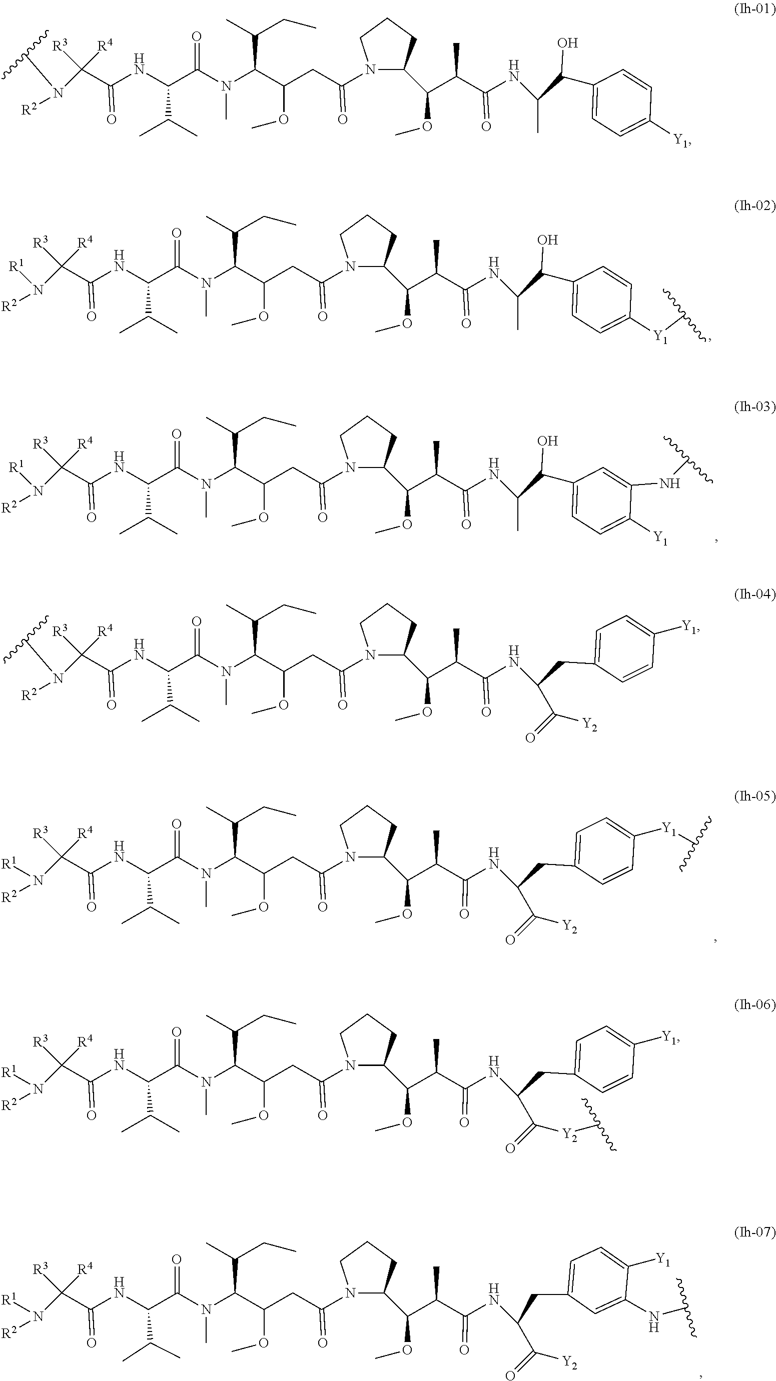 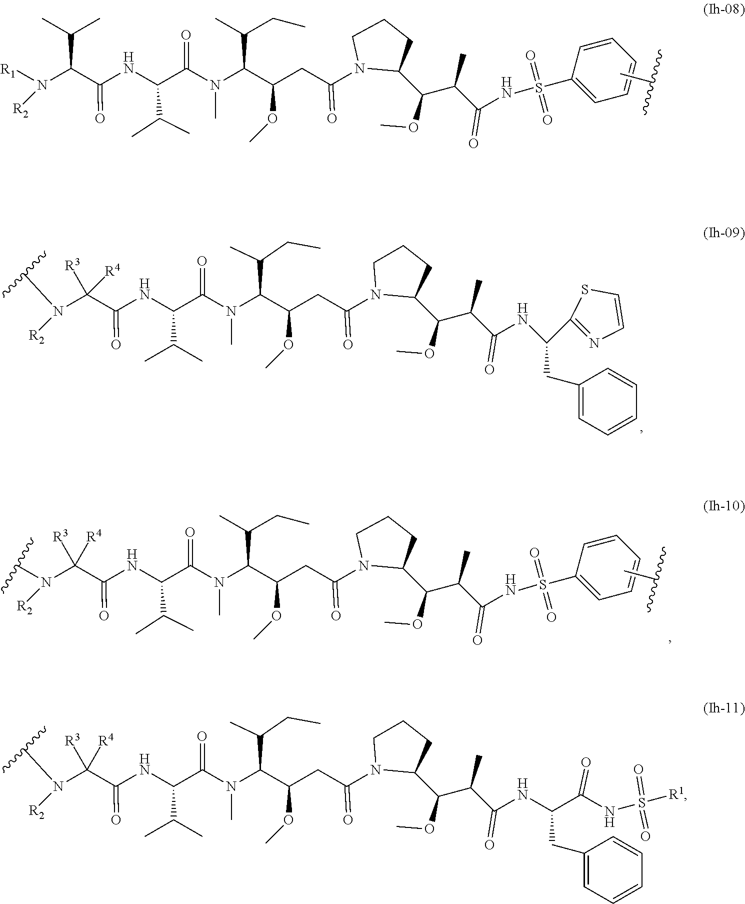
or a derivative with one or more isotopes; or a pharmaceutically acceptable salt, hydrate, or hydrated salt thereof; or a polymorphic crystalline structure thereof; or an optical isomer, racemate, diastereomer or enantiomer thereof;
wherein R¹, R², R³, R⁴ and R⁵ are independently H; C₁-C₈ linear or branched alkyl, aryl, heteroaryl, heteroalkyl, alkylcycloalkyl, ester, ether, amide, amine, heterocycloalkyl, or acyloxylamine; or peptide containing 1-8 amino acids, or polyethyleneoxy unit having formula (OCH₂CH₂)ₚ or (OCH₂CH(CH₃))ₚ, wherein p is an integer from 1 to about 5000; the two Rs: R¹ and R², R² and R³, R¹ and R³ or R³ and R⁴ can form a 3-8 member cyclic ring of alkyl, aryl, heteroaryl, heteroalkyl, or alkylcycloalkyl group;
X₃ is H, CH₃ or X₁′R₁′, wherein X₁′ is NH, N(CH₃), NHNH, O, or S, and R₁′ is H or C₁-C₈ linear or branched alkyl, aryl, heteroaryl, heteroalkyl, alkylcycloalkyl, or acyloxylamine;
R₃′ is H or C₁-C₆ linear or branched alkyl;
Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, NR₁R₂R₃;
Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) or C(O)NR₁ when linked to a connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH or C(O)NHR₁ when not linked to the connecting site “ [symbol] ”;
R₁₂ is OH, NH₂, NHR₁, NHNH₂, NHNHCOOH, O—R₁—COOH, NH—R₁—COOH, NH—(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-5000; R₁, R₂, R₃, R₄, R₅, R₅′, Z₁, Z₂, and n are the same as defined above;
(j) the Hemiasterlin and its analogues have the following formula: 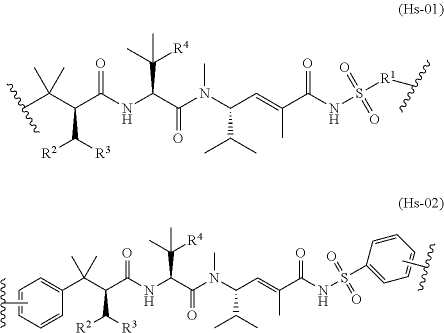
wherein R¹, R², R³, R⁴ and R⁵ are independently H; C₁-C₈ linear or branched alkyl, aryl, heteroaryl, heteroalkyl, alkylcycloalkyl, ester, ether, amide, amine, heterocycloalkyl, or acyloxylamine; or peptide containing 1-8 aminoacids, or polyethyleneoxy unit having formula (OCH₂CH₂)ₚ or (OCH₂CH(CH₃))ₚ, wherein p is an integer from 1 to about 5000; the R² and R³ can form 3-8 member cyclic ring of alkyl, aryl, heteroaryl, heteroalkyl, or alkylcycloalkyl group;
(k) the Eribulin has the following formula: 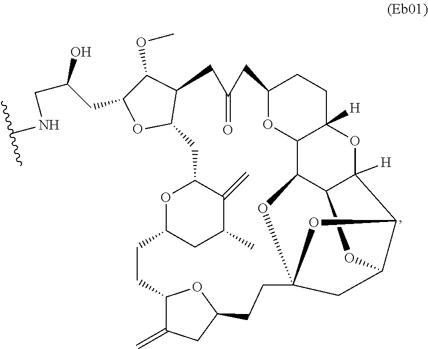
wherein [symbol] is a linkage site that links to L₁ and/or L₂ independently;
(l) the Inhibitor of nicotinamide phosphoribosyltransferases (NAMPT) have the following formula, NP01, NP02, NP03, NP04, NP05, NP06, NP07, NP08, and NP09: 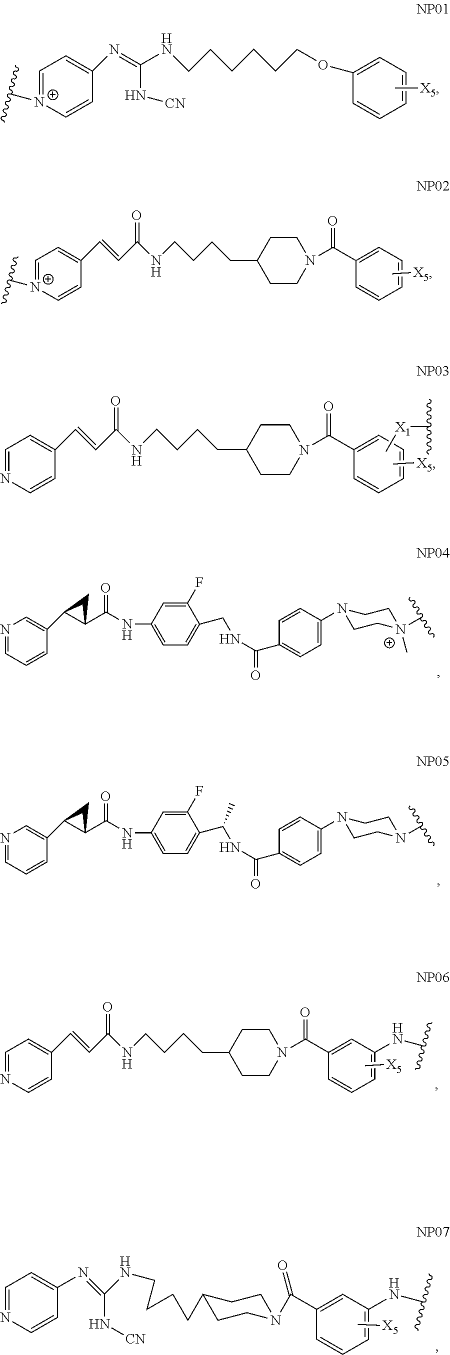 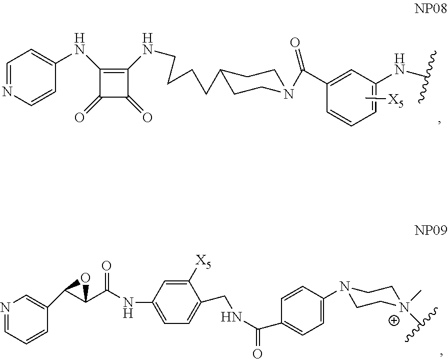
or a derivative with one or more isotopes, or a pharmaceutically acceptable sals, hydrate, or hydrated salt thereof; or a polymorphic crystalline structure thereof; or an optical isomer, racemate, diastereomer or enantiomer thereof; wherein “ [symbol] ” is the same above; X₅ is F, Cl, Br, I, OH, OR₁, R₁, OPO₃H₂, OSO₃H, NHR₁, OCOR₁, or NHCOR₁;
(m) the benzodiazepine dimer and its analogs have the following formulae, PB01, PB02, PB03, PB04, PB05, PB06, PB07, PB08, PB09, PB10, PB11, PB12, PB13, PB14, PB15, and PB16: 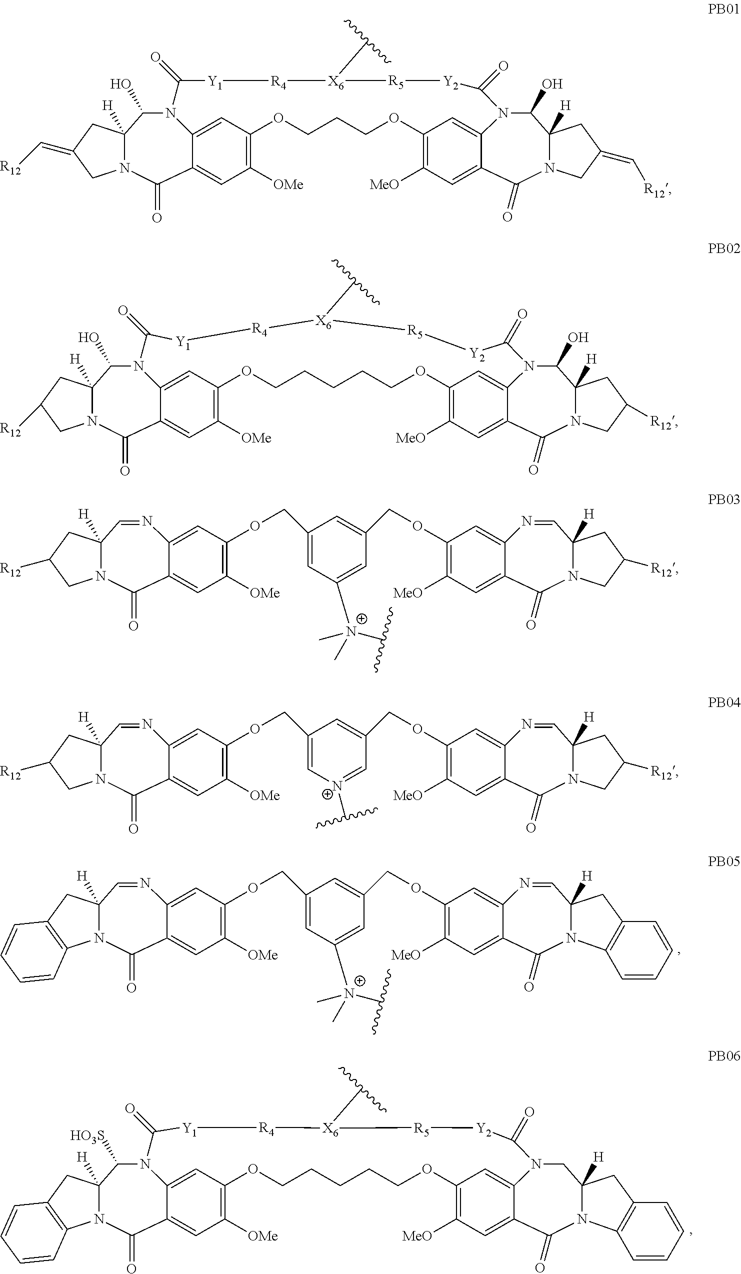 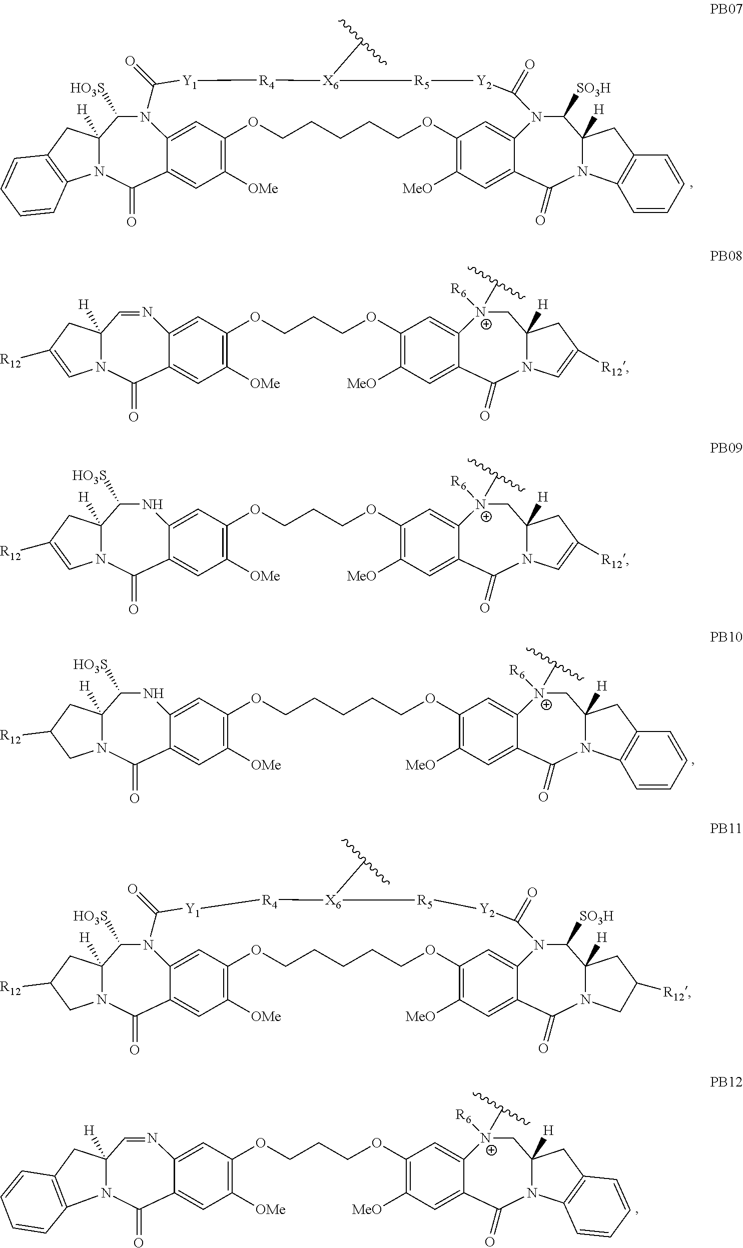 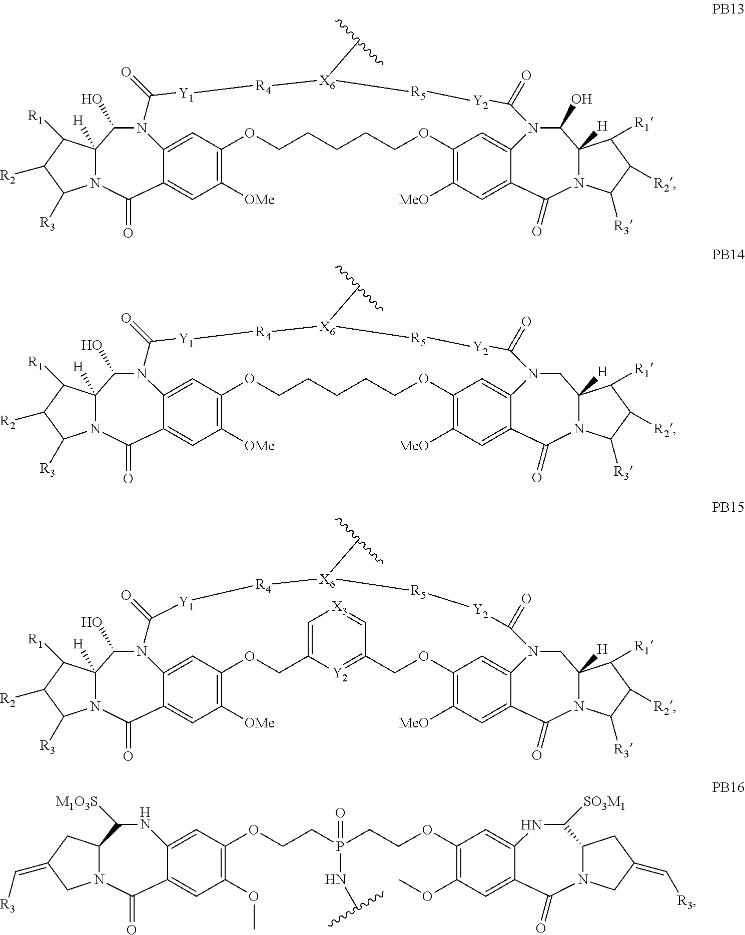
or a derivative with one or more isotopes, or a pharmaceutically acceptable salt, hydrate, or hydrated salt thereof; or a polymorphic crystalline structure thereof; or an optical isomer, racemate, diastereomer or enantiomer thereof;
wherein X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁;
R¹, R², R³, R^{1′}, R^{2′}, and R^{3′} are independently H; F; CL; ═O; ═S; OH; SH; C₁-C₈ linear or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amine (NHR₅, NR₅R₅′), heterocycloalkyl, or acyloxylamine (—C(O)NHOH, —ONHC(O)R₅); or peptide containing 1-20 natural or unnatural aminoacids, or polyethyleneoxy unit of formula (OCH₂CH₂)ₚ or (OCH₂CH(CH₃))ₚ, wherein p is an integer from 1 to about 1000, the two Rs: R¹ and R², R² and R³, R¹ and R³, R^{1′} and R^{2′}, R^{2′} and R^{3′}, or R^{1′} and R^{3′} can independently form a 3-8 member cyclic ring of alkyl, aryl, heteroaryl, heteroalkyl, or alkylcycloalkyl group;
X₃ and Y₃ are independently N, NH, CH or CR₁, or one of X₃ and Y₃ can be absent;
R₄, R₅, R₆, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, or acyloxylamines;
X₆ is CH, C(O), N, P(O)NH, P(O)NR₁, CHC(O)NH, C₁-C₈ linear or branched alkyl, or heteroalkyl; C₃-C₈ aryl, heteroaryl, alkylcycloalkyl, acyloxyl, alkylaryl, alkylaryloxyl, or alkylarylamino, or an Aa (amino acid);
Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, NR₁R₂R₃;
“ [symbol] ” is the same as defined above;
(n) the CC-1065 analog and doucarmycin analogs have the following formula, CC01, CC02 CC03 CC04 CC05 CC06 and CC07: 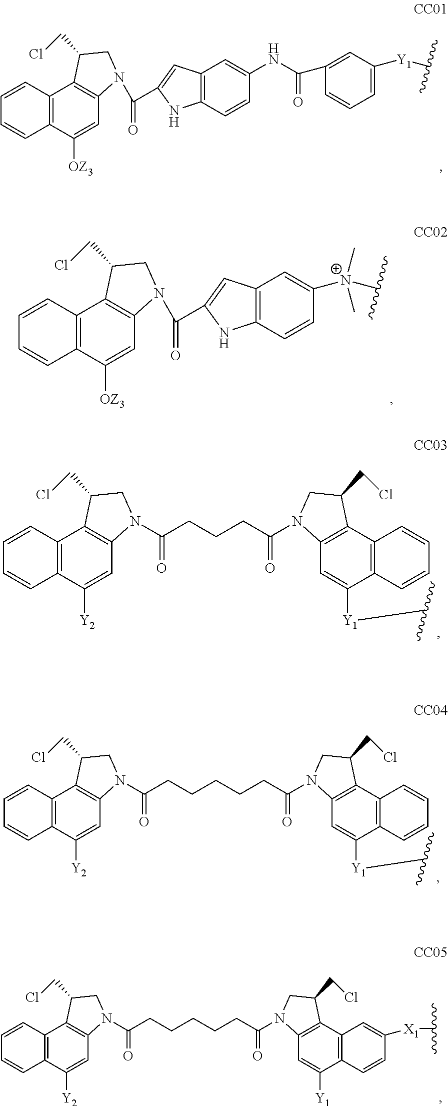 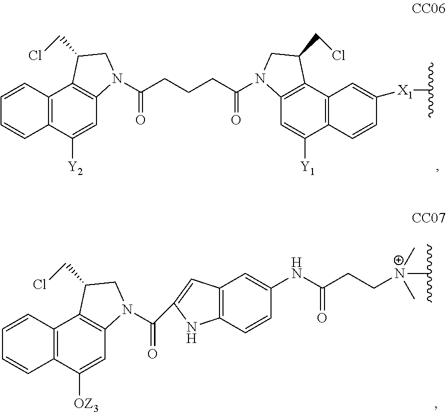
wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) or C(O)NR₁ when linked to a connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH or C(O)NHR₁ when not linked to the connecting site “ [symbol] ”;
Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are the same as defined above;
(o) the amatoxin and its analogs have the following formula, Am01, Am02, and Am03: 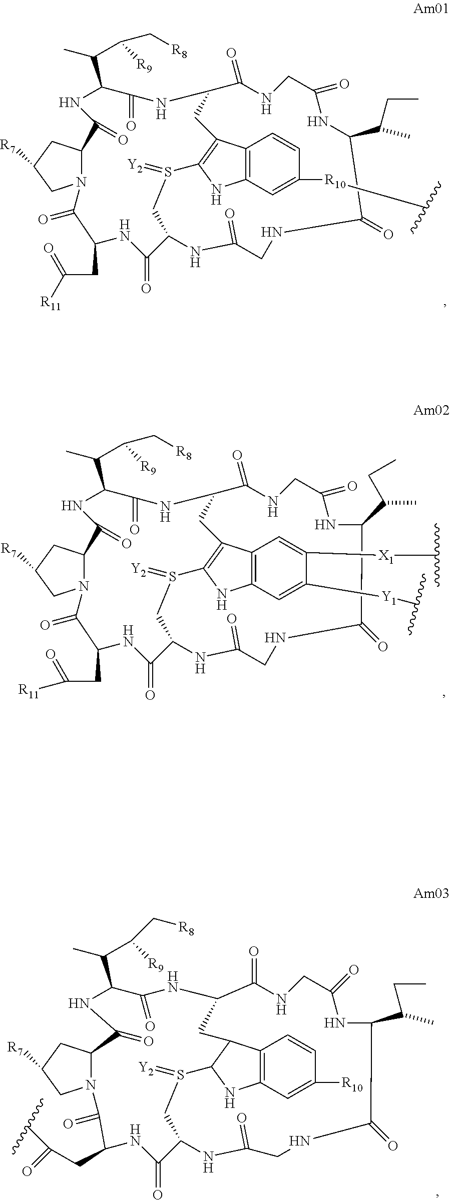
or a derivative with one or more isotopes, or a pharmaceutically acceptable salt, hydrate, or hydrated salt thereof; or a polymorphic crystalline structure thereof; or an optical isomer, racemate, diastereomer or enantiomer thereof;
wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) or C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH—(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa)ᵣ is the same or different sequence of 1-8 amino acids; n and m₁ are independently 1-20; p is 1-1000; R₁, R₂, Ar, and “ [symbol] ” are the same as defined above;
(p) the Spliceostatins and pladienolides are spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)-4-hydroxy-1, 6-dioxaspiro[2.5]oct-5-yl]-3-methylpenta-2, 4-dien-1-y-1}-2,5-dimethyltetrahydro-2H-pyran-3-yl]amino}-5-oxopent-3-en-2-yl acetate, Pladienolide B, Pladienolide D, and E7107 having a core structure of Sp-01: 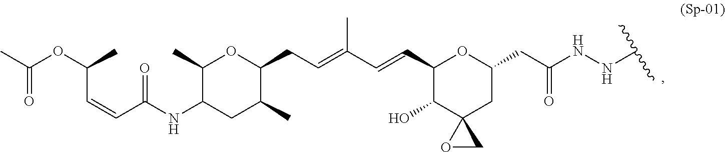 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]
(q) the protein kinase inhibitors are selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, and Nazartinib (EGF816), having the following formula, PK01-PK40: 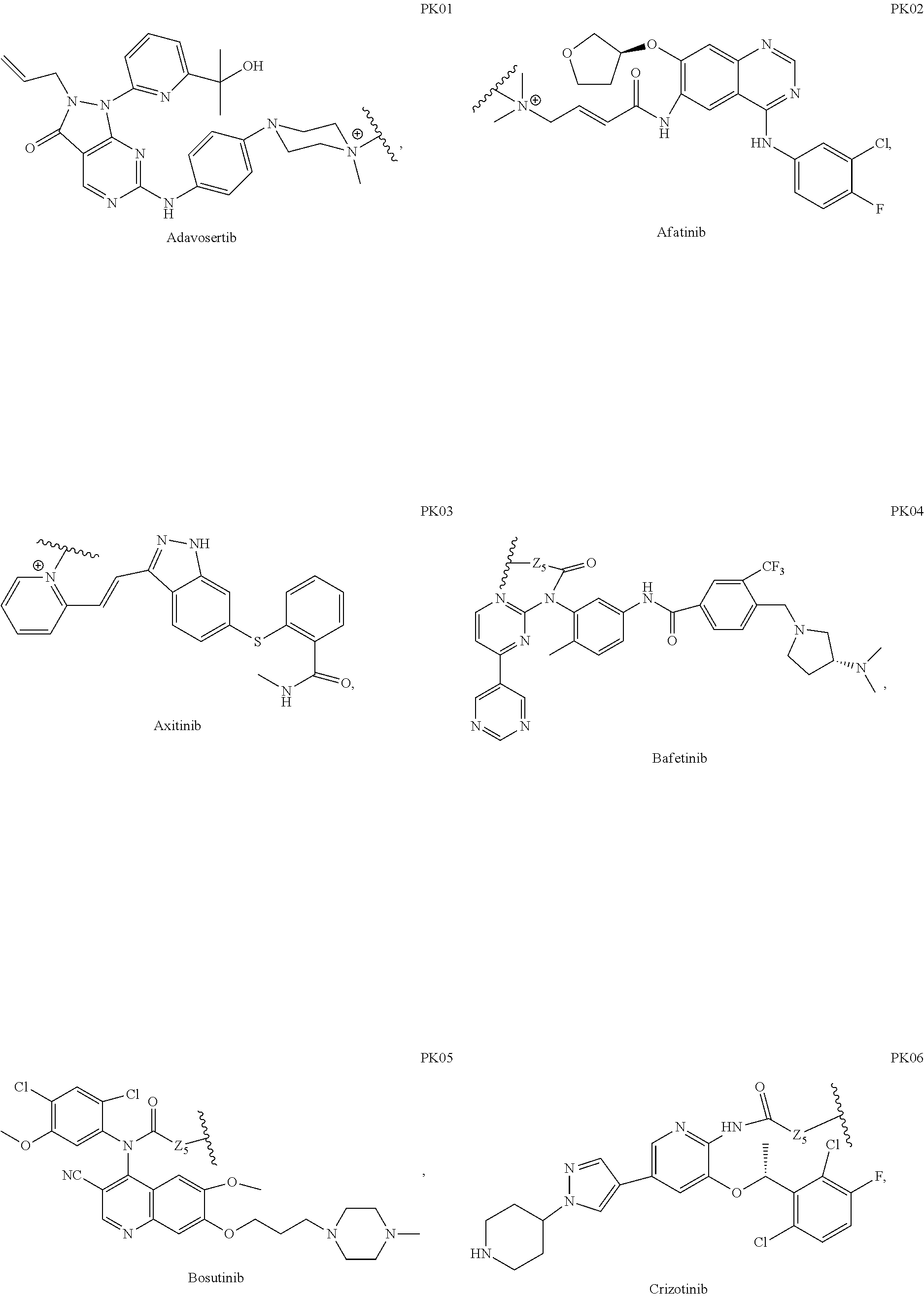 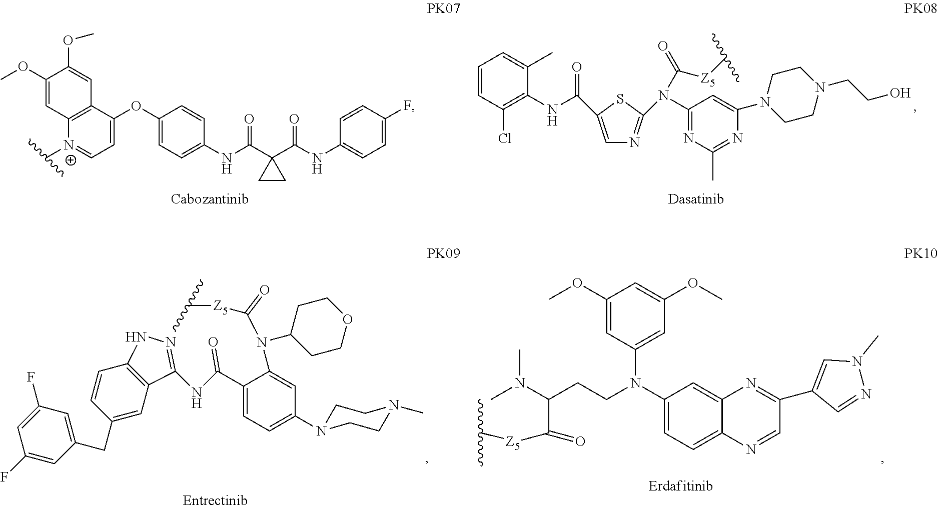 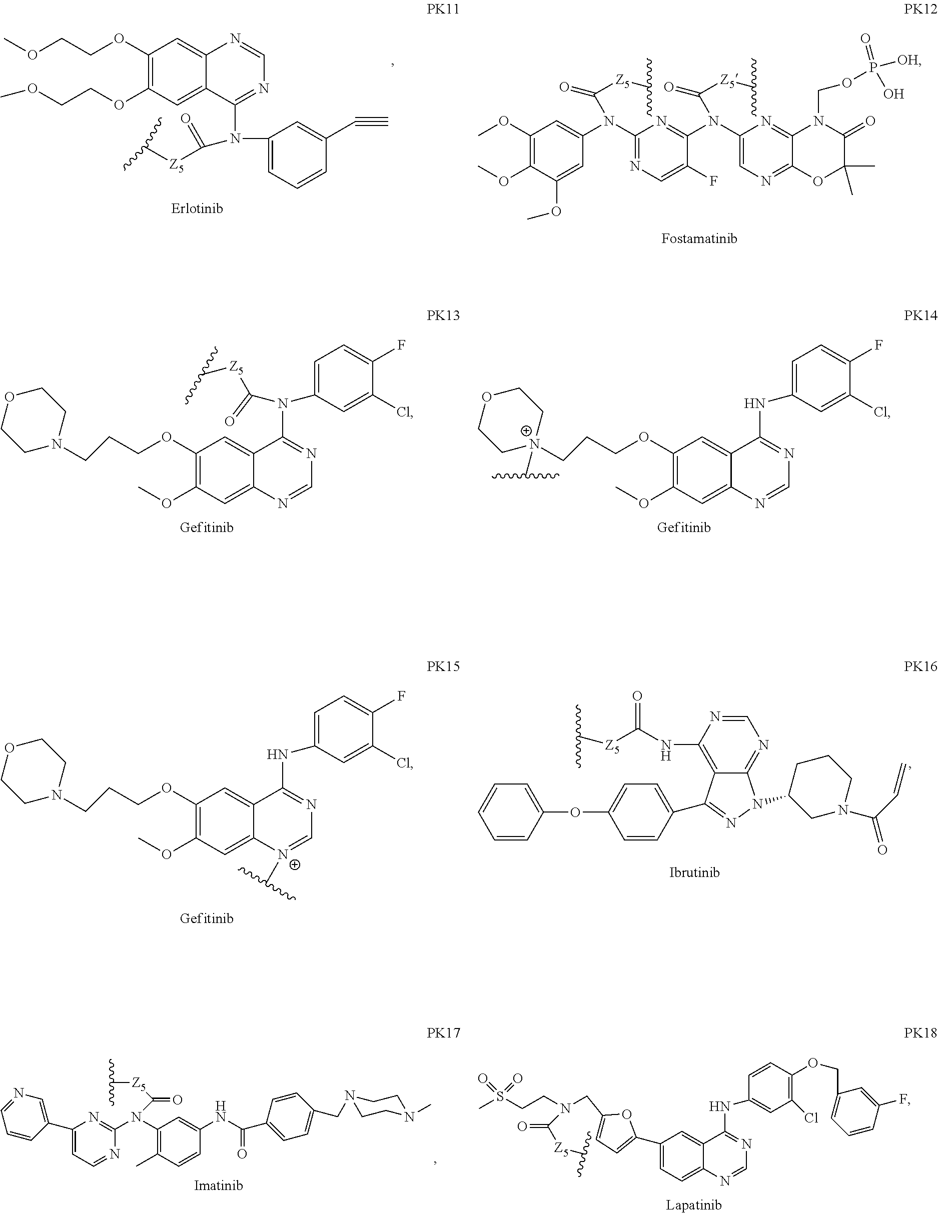 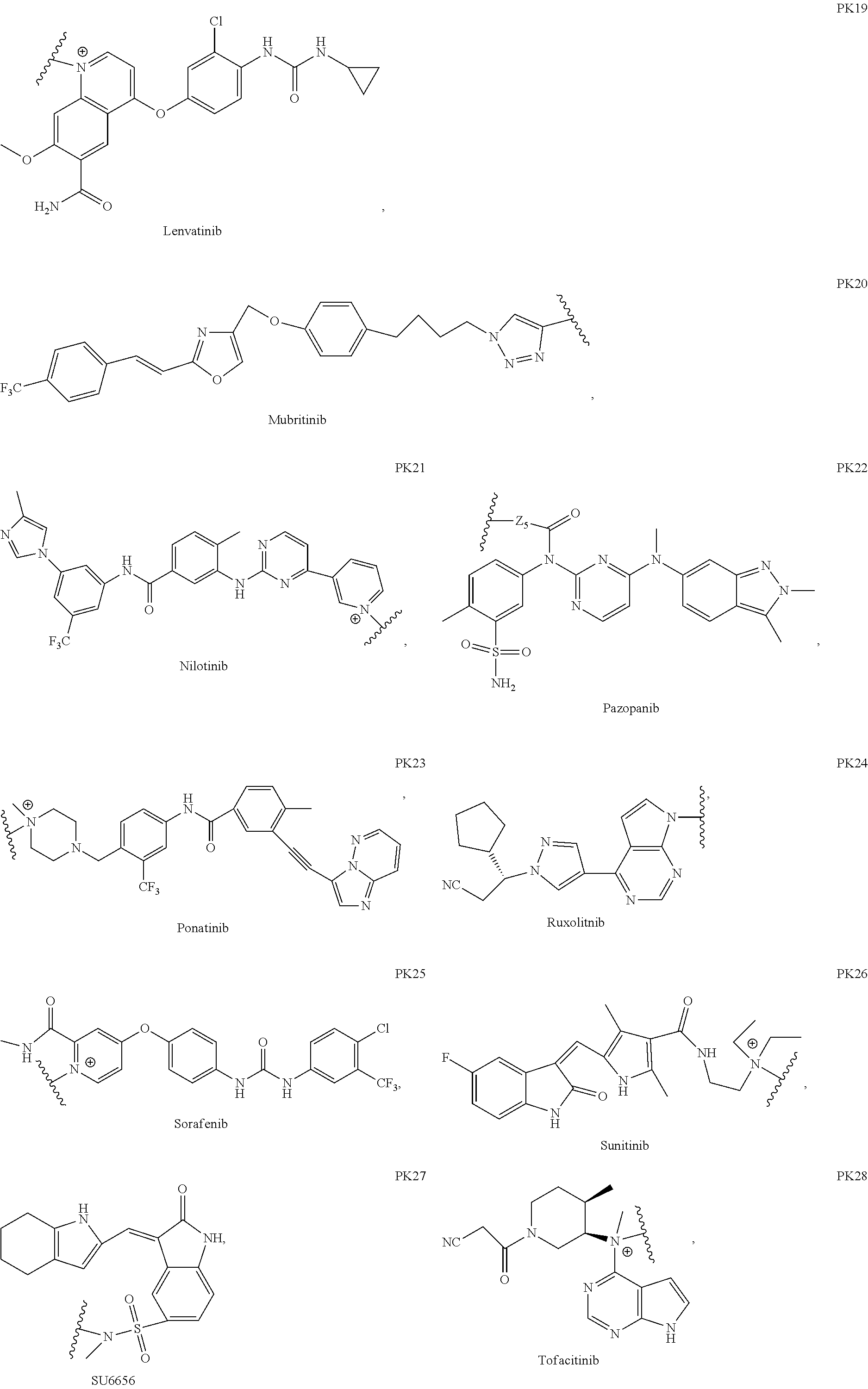 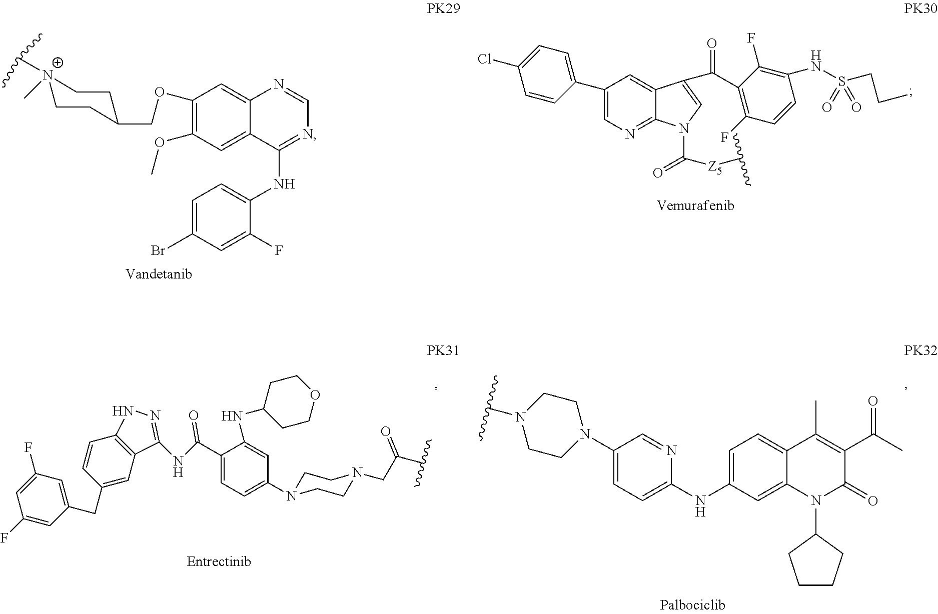 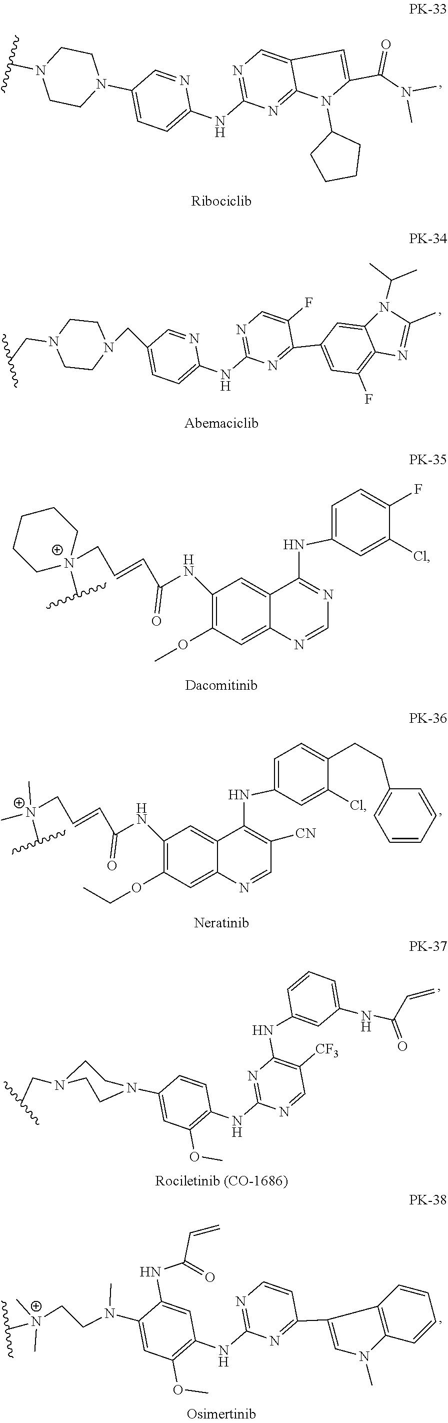 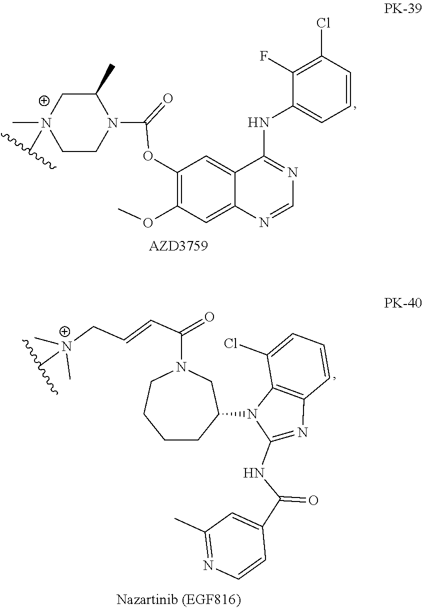
wherein Z₅ and Z₅′ are independently O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) or C(O)NR₁; R₁ and R₂ are defined the same above;
(r) the MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733, and have the following formula: 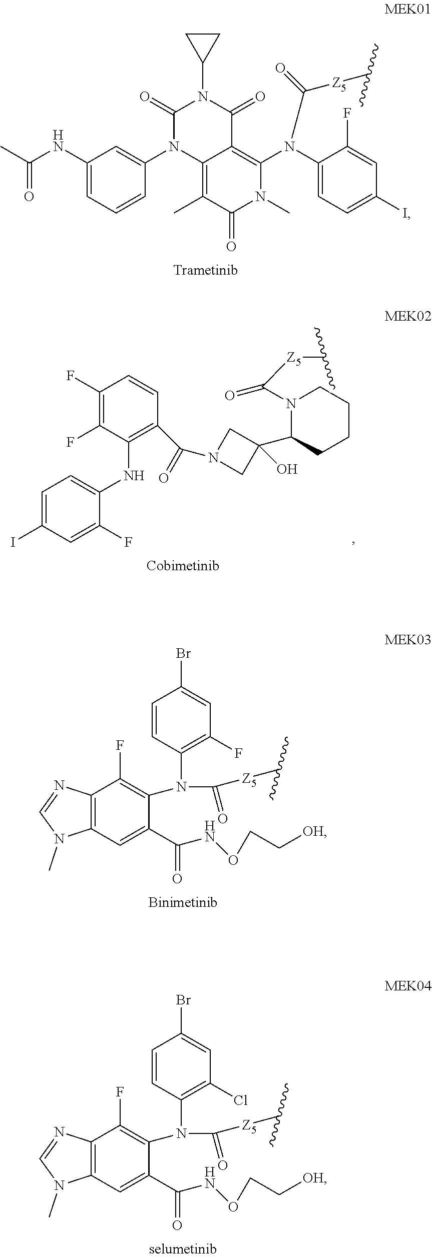
wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁; R₁ and R₂ are the same as defined above;
(s) the proteinase inhibitors are selected from Carfilzomib, Clindamycin, Retapamulin, Indibulin, having the following formulae: 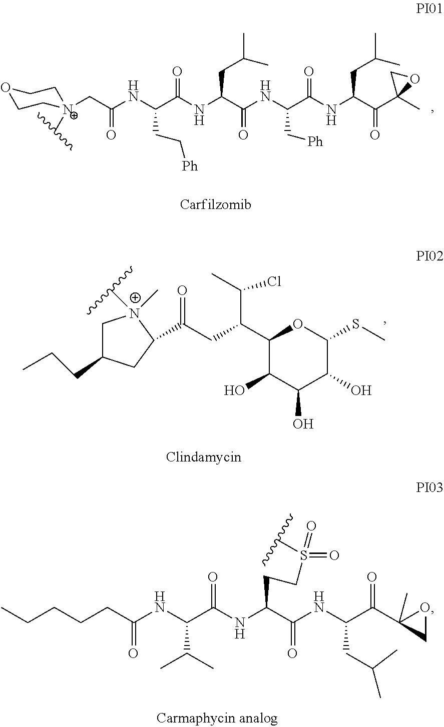
(t) the immunotoxin are selected from Diphtheria toxin (DT), Cholera toxin (CT), Trichosanthin (TCS), Dianthin, Pseudomonas exotoxin A (ETA), Erythrogenic toxins, Diphtheria toxin, AB toxins, Type III exotoxins, proaerolysin, and topsalysin;
(u) the cell receptor agonist or stimulating molecule are selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y₁-Y₆); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones selected from the group consisting of luteinizing hormone-releasing hormone (LHRH) agonists and antagonists, and gonadotropinrreleasing hormone (GnRH) agonist selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gy-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of αVβ₁, αVβ₃, αVβ₅, αVβ₆, α₆β₄, α7β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists;
the cell receptor agonist is selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αvβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR2 for a Toll-like receptor,), LB23 (for an androgen receptor), LB24 (Cilengitide/cyclo(-RGDfV-) for an av integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) having following structures: 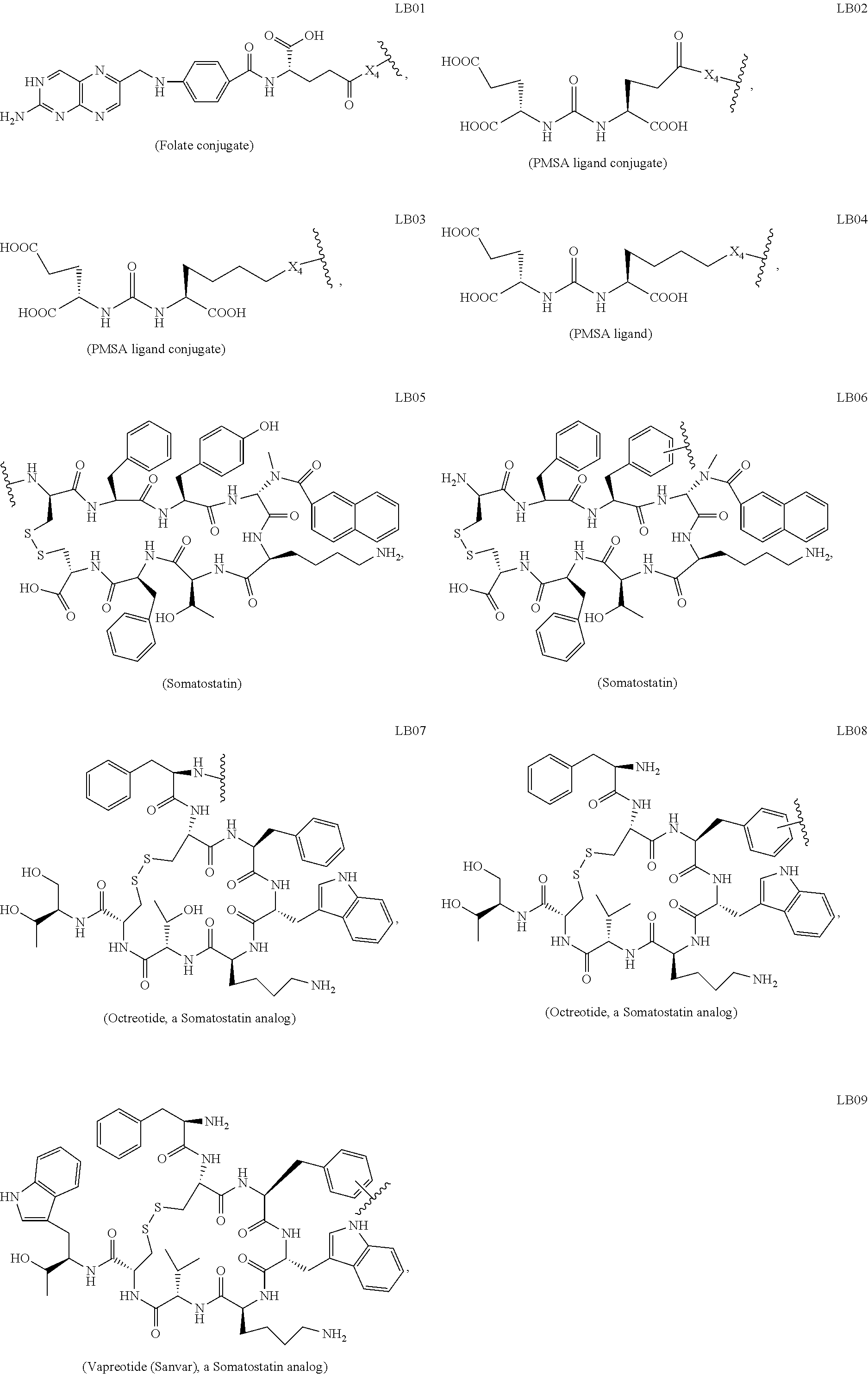 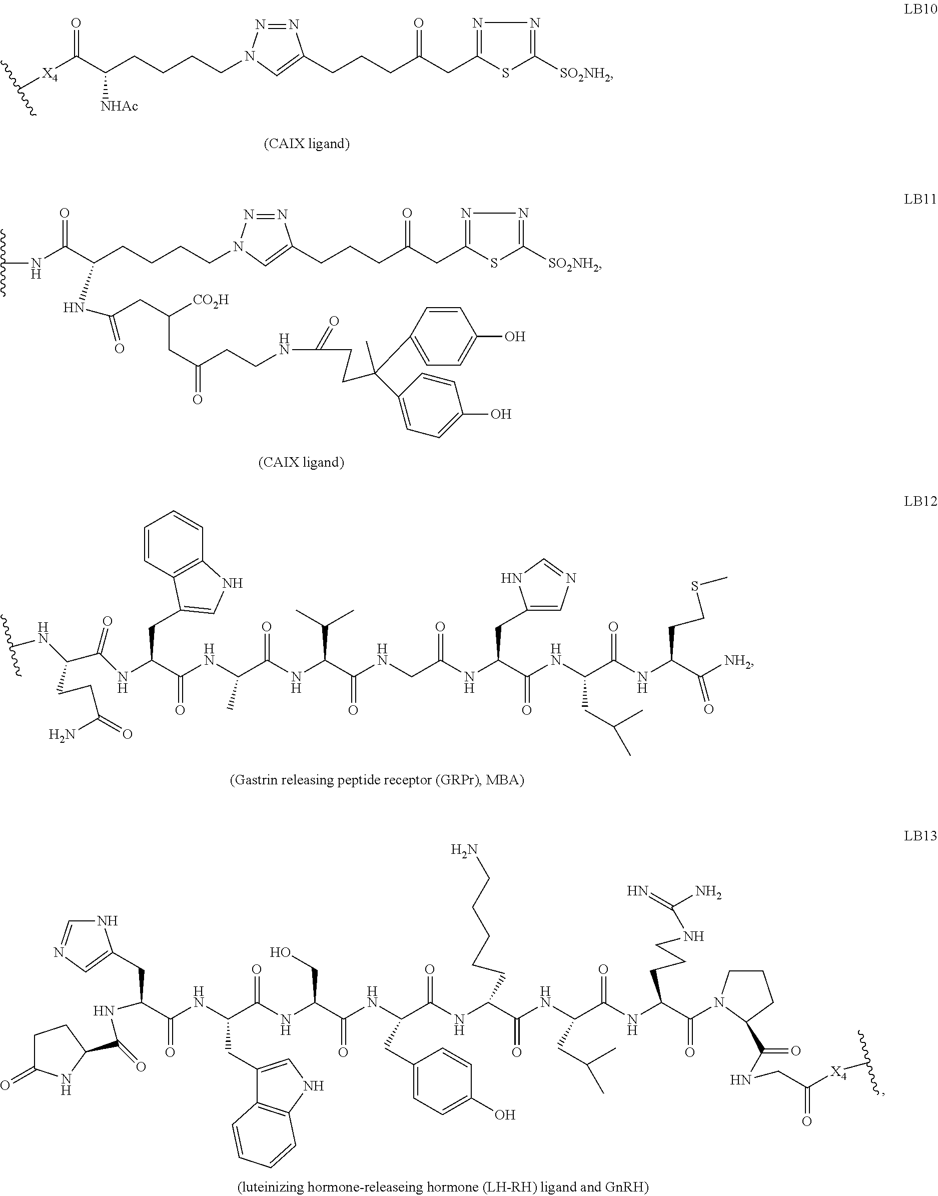 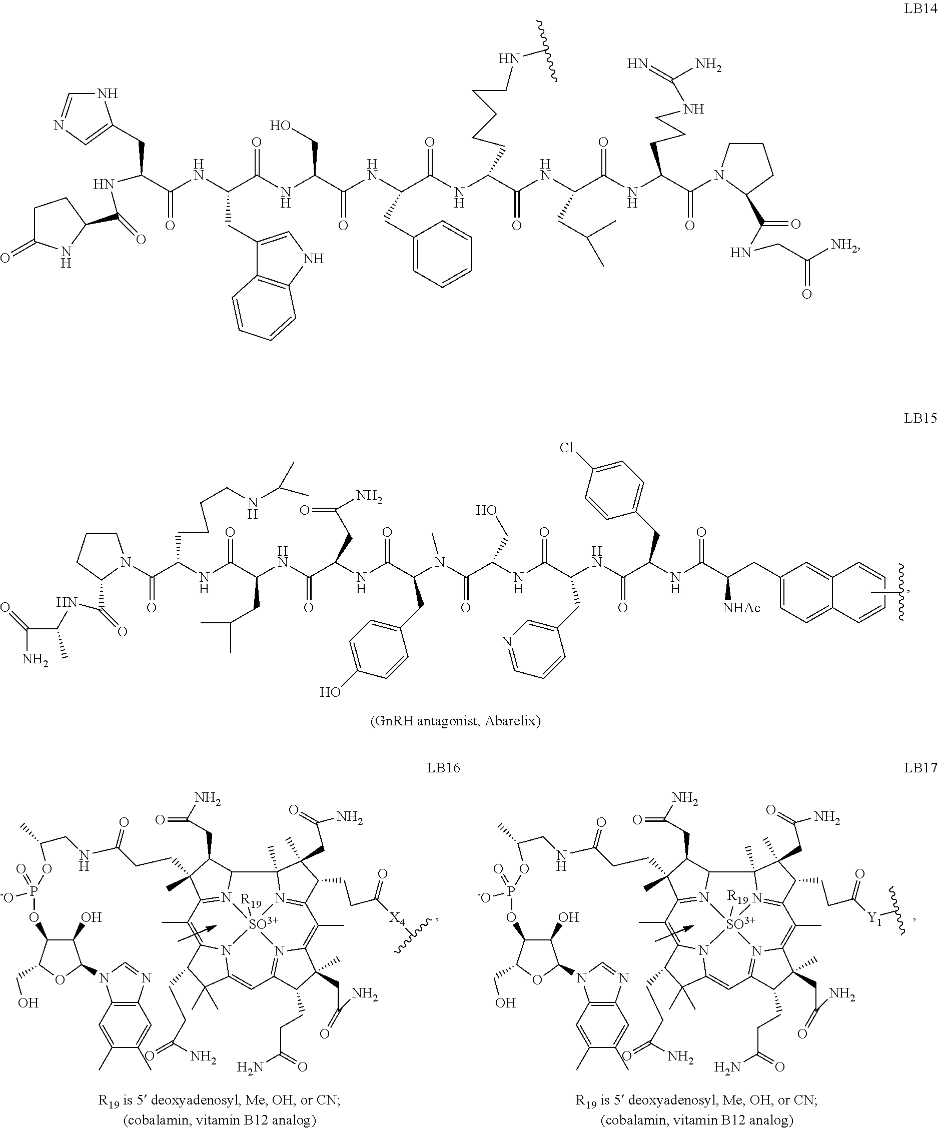 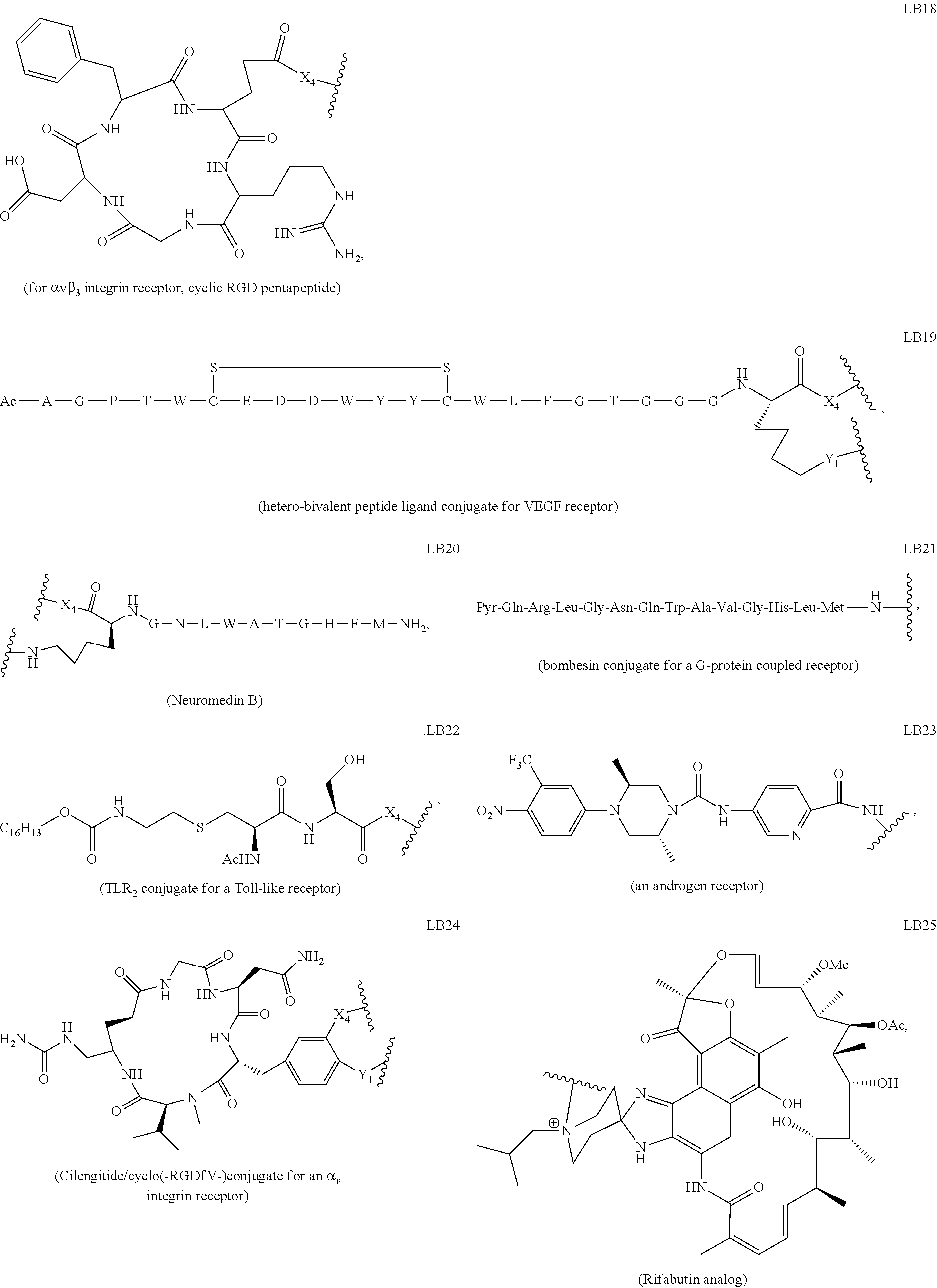 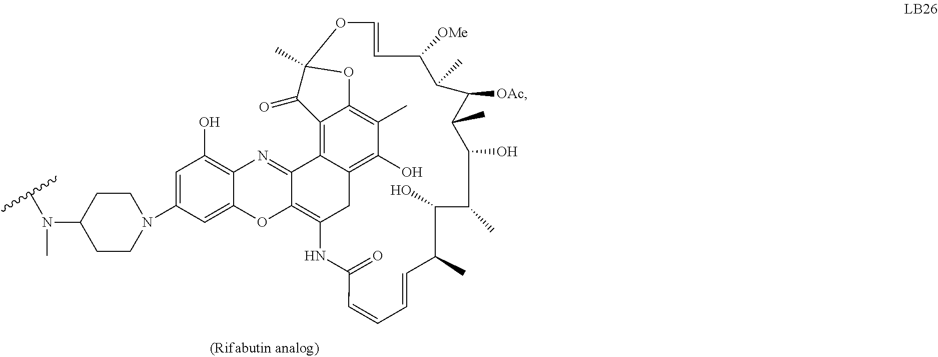 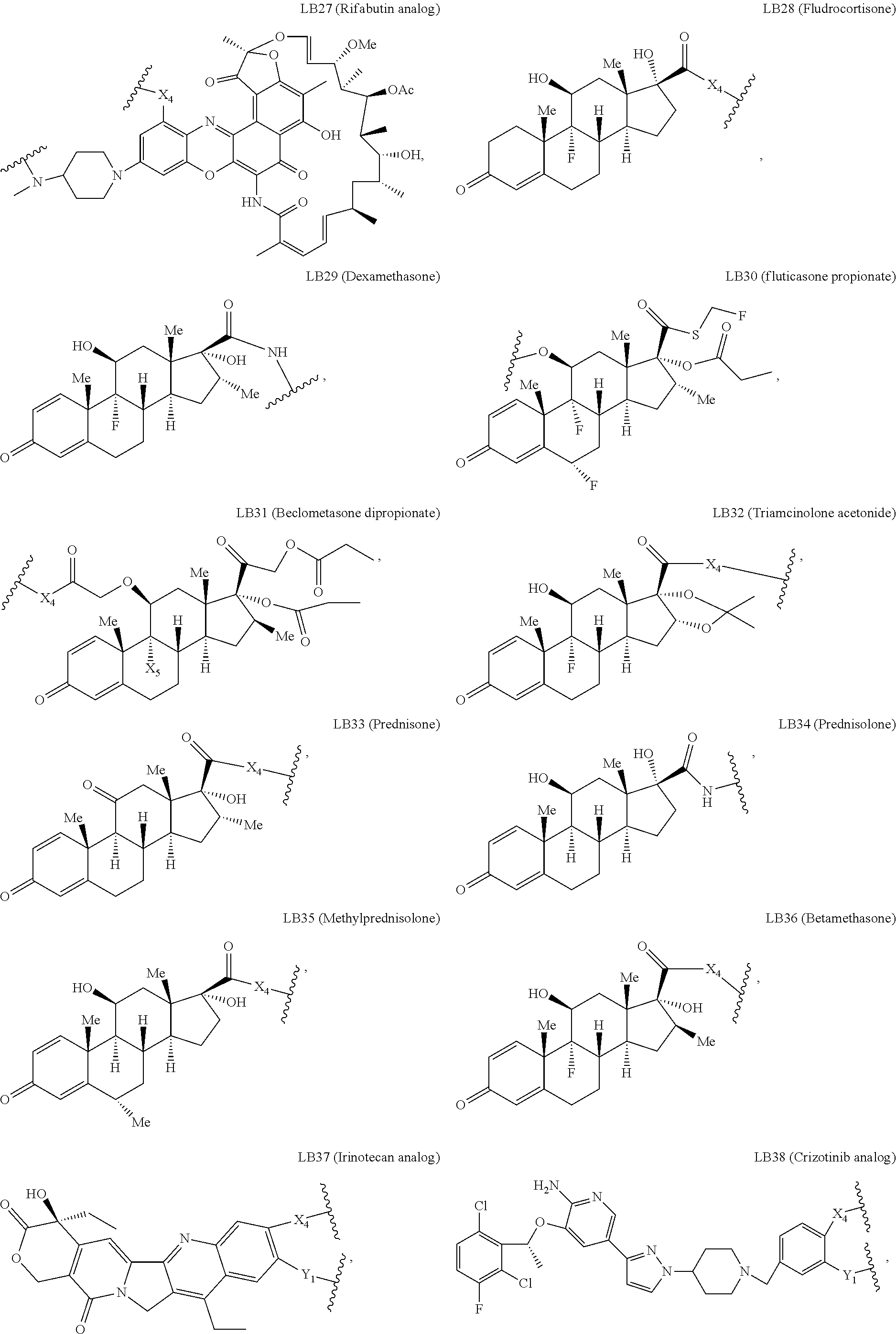 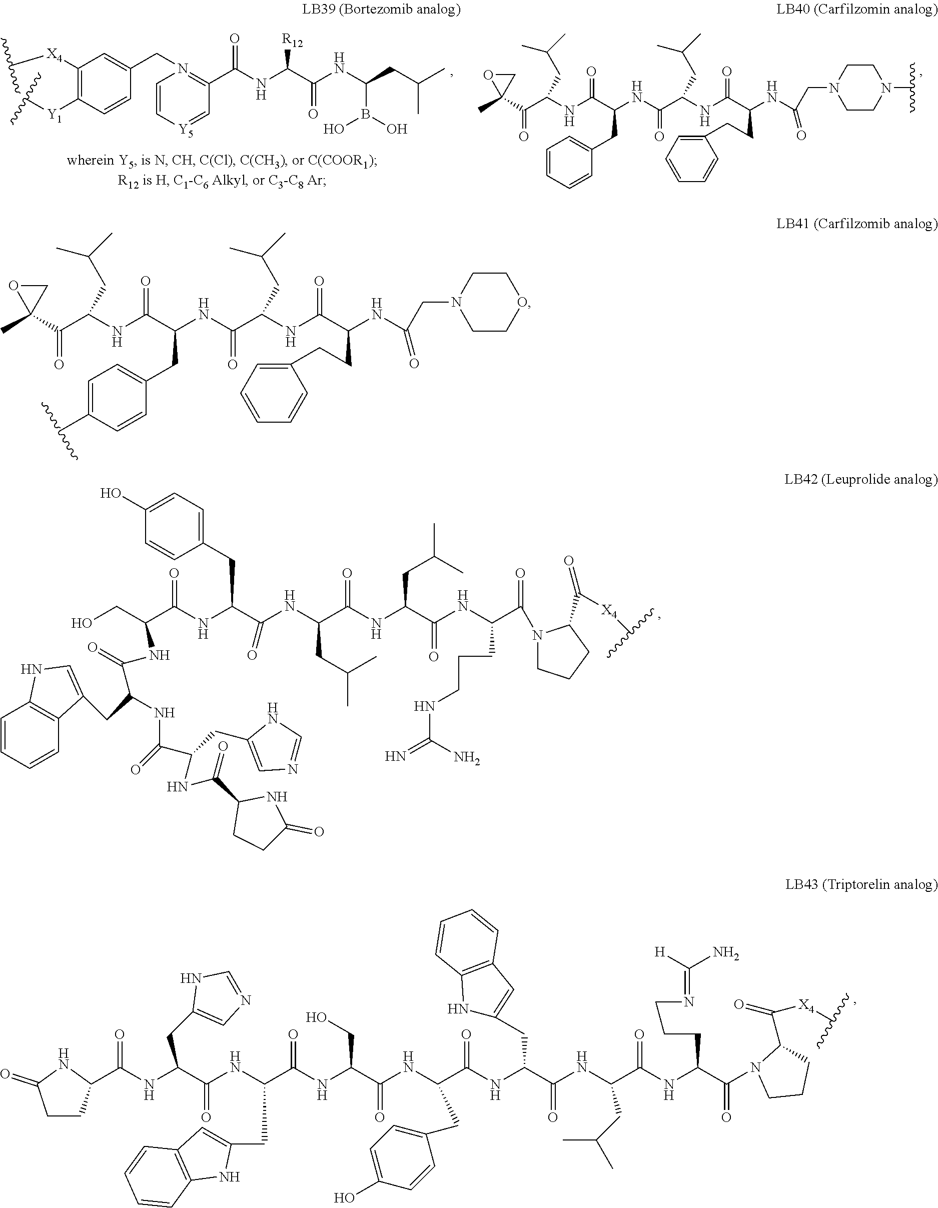 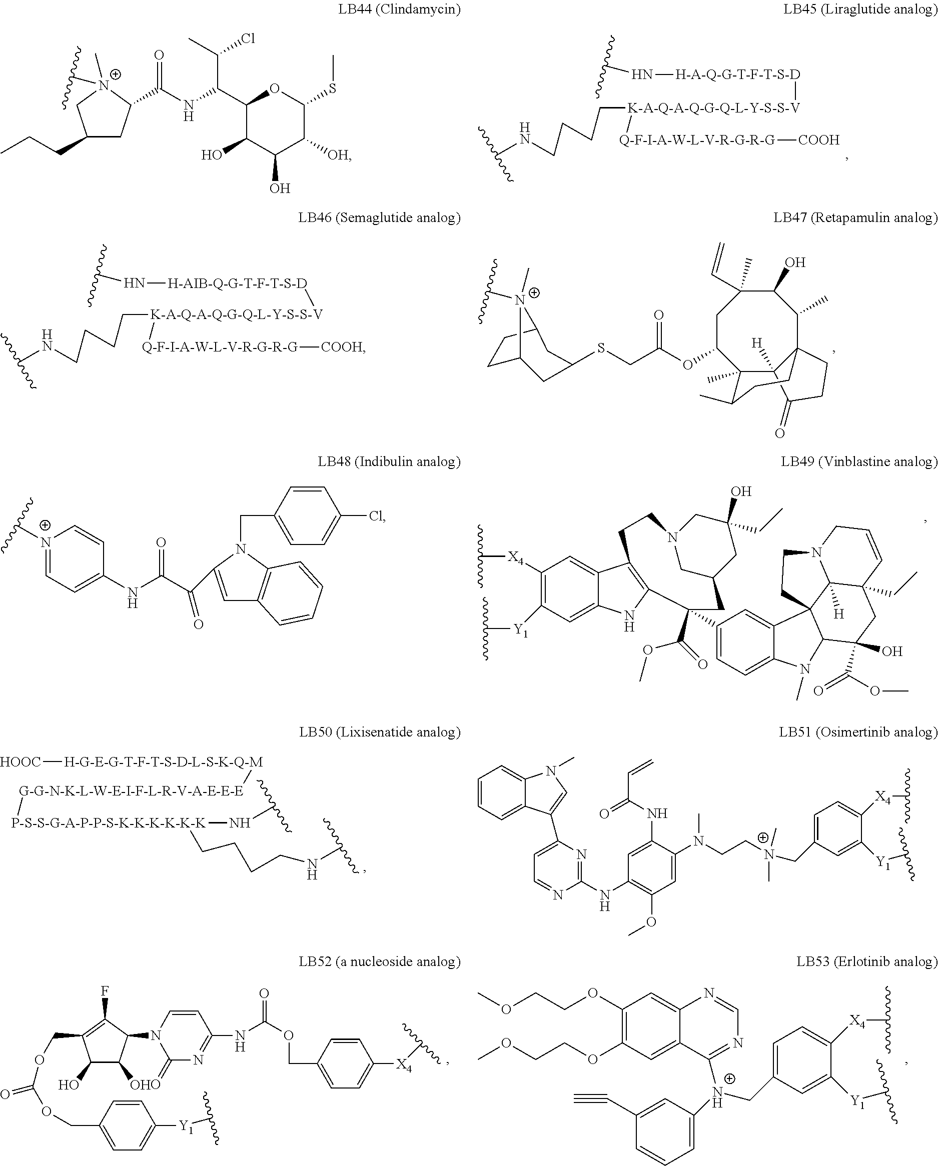 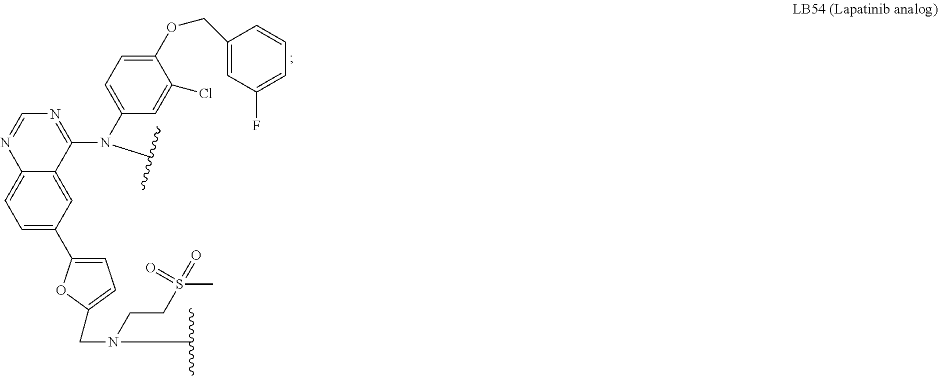
wherein X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) or C(O)NR₁;
(v) the one, two or more DNA, RNA, mRNA, small interfering RNA (siRNA), microRNA (miRNA), and PIWI interacting RNAs (piRNA) have a structure of: 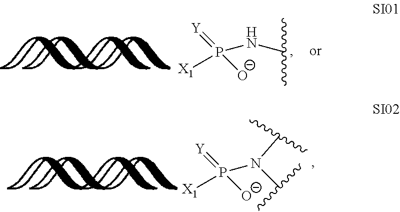
wherein “ [symbol] ” is a site to link the side chain linker; [symbol] is single or double strands of DNA, RNA, mRNA, siRNA, miRNA, or piRNA; X₁, and Y are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) or C(O)NR₁.

34. The process according to claim 23, wherein the antibody or antibody-like protein is selected from:
(A): the group consisting of probody, nanobody, peptides, a polymeric micelle, a liposome, a lipoprotein-based drug carrier, a nano-particle drug carrier, a dendrimer, and a molecule or a particle said above coating or linking with a cell-binding ligand, or a combination of two or more of the above;
(B): the group consisting of a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune antibody-like proteins (SIP), a lymphokine antibody-like protein, a hormone antibody-like protein, a growth factor antibody-like protein, a colony stimulating factor antibody-like protein, a nutrient-transport antibody-like protein, large molecular weight antibody-like proteins, fusion antibody-like proteins, kinase inhibition antibody-like protein, gene-targeting antibody-like protein, antibody-like protein coated on nanoparticles or polymers modified with antibodies or large molecular weight antibody-like proteins; or
(C): the group consisting of an IgG antibody that is able to bind against tumor cells, virus infected cells, microorganism infected cells, parasite infected cells, autoimmune disease cells, activated tumor cells, myeloid cells, activated T-cells, an affecting B cells, or melanocytes, or abnormal cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11(C—C motif chemokine 11), CCR4 (C—C chemokine receptor type 4), CCR5, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C—X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ₃, α₄β₇, α5β1, α6β4, α7β7, αllβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-1 (Trop1), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

35. The process according to claim 28, wherein the conjugate, in which the drugs are conjugated to disulfide bond sites between heavy-light chains of an antibody has one of the structures of a001-a233, C-009, C-020, C-025, C-027, C-031, C-037, C-038, C-039, C-043, C-046, C-052, C-056, C-059, C-063, C-066, C-071, C-079, C-084, C-087, C-093, C-096, C-102, C-109, C-111, C-118, C-123, C-133, C-143, C-155, C-168, C-172, C-182, C-186, C-198, C-203, C-208, C-214, C-215, C-216, C-217, C-218, C-226, C-227, C-231, C-237, C-249, C-259, C-260, C-261, C-325, C-326, C-327, C-328, C-329, C-330, C331, C-332, C-333, C-334, C-335, C-353, C-363, C-371, C-378, C-379, C-380, C-381, C-384, C-385, C-386, C-387, C-397, C-400, C-403, C-406, C-408a, C-408b, C-408c, C-408d, C-408e, C-408f, C-410a, C-410b, C-410c, C-412a, C-412b, C-412c, C-413a, C-413b, C-413c, C-413d, C-416a, C-416b, C-416c, C-416d, C-422a, C-422b, C-422c, C-422d, C-425a, C-425b, C-425c, C-425d, C-428a, C-428b, C-428c, C-428d, C-431a, C-431b, C-431c, C-431d, as illustrated below: 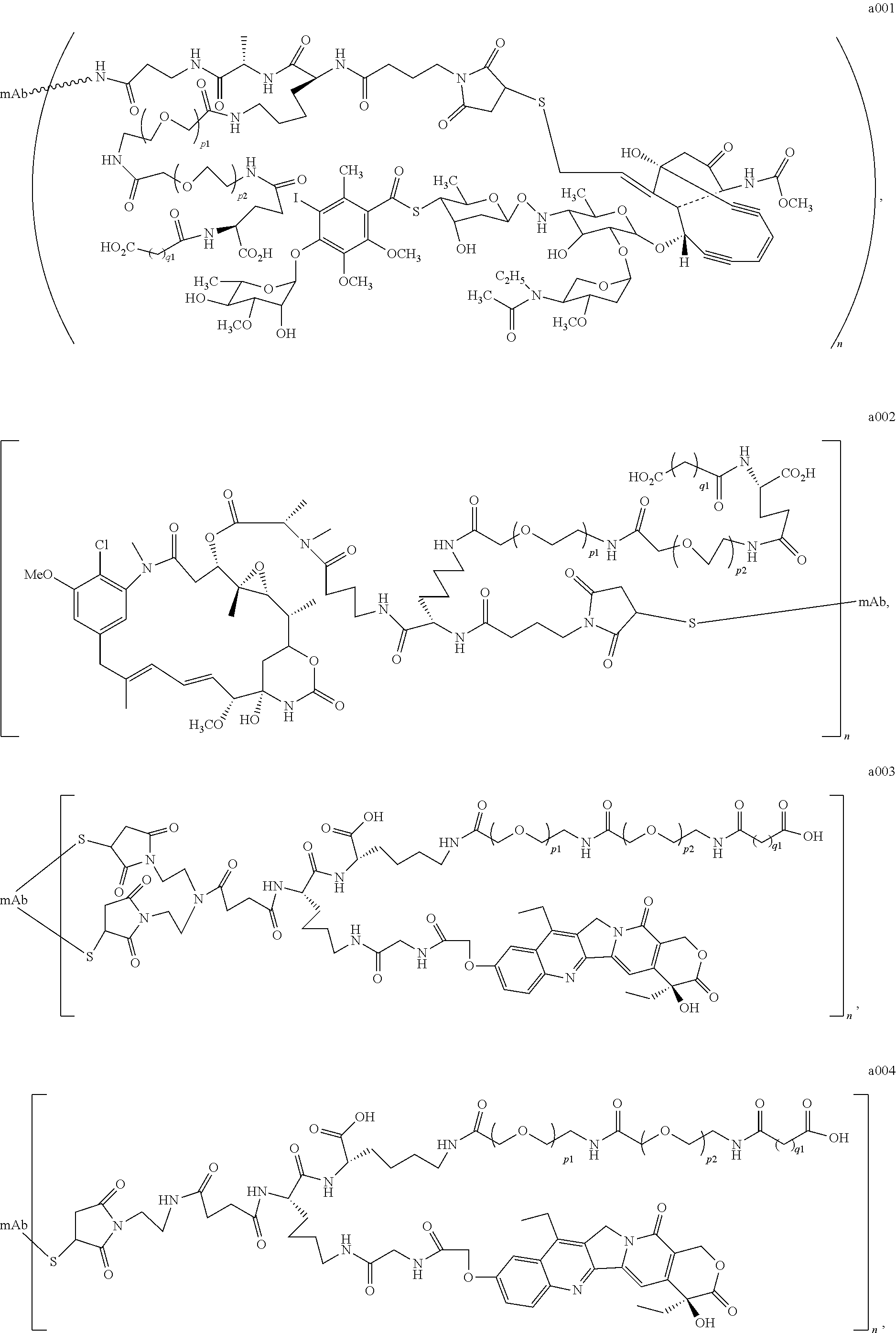 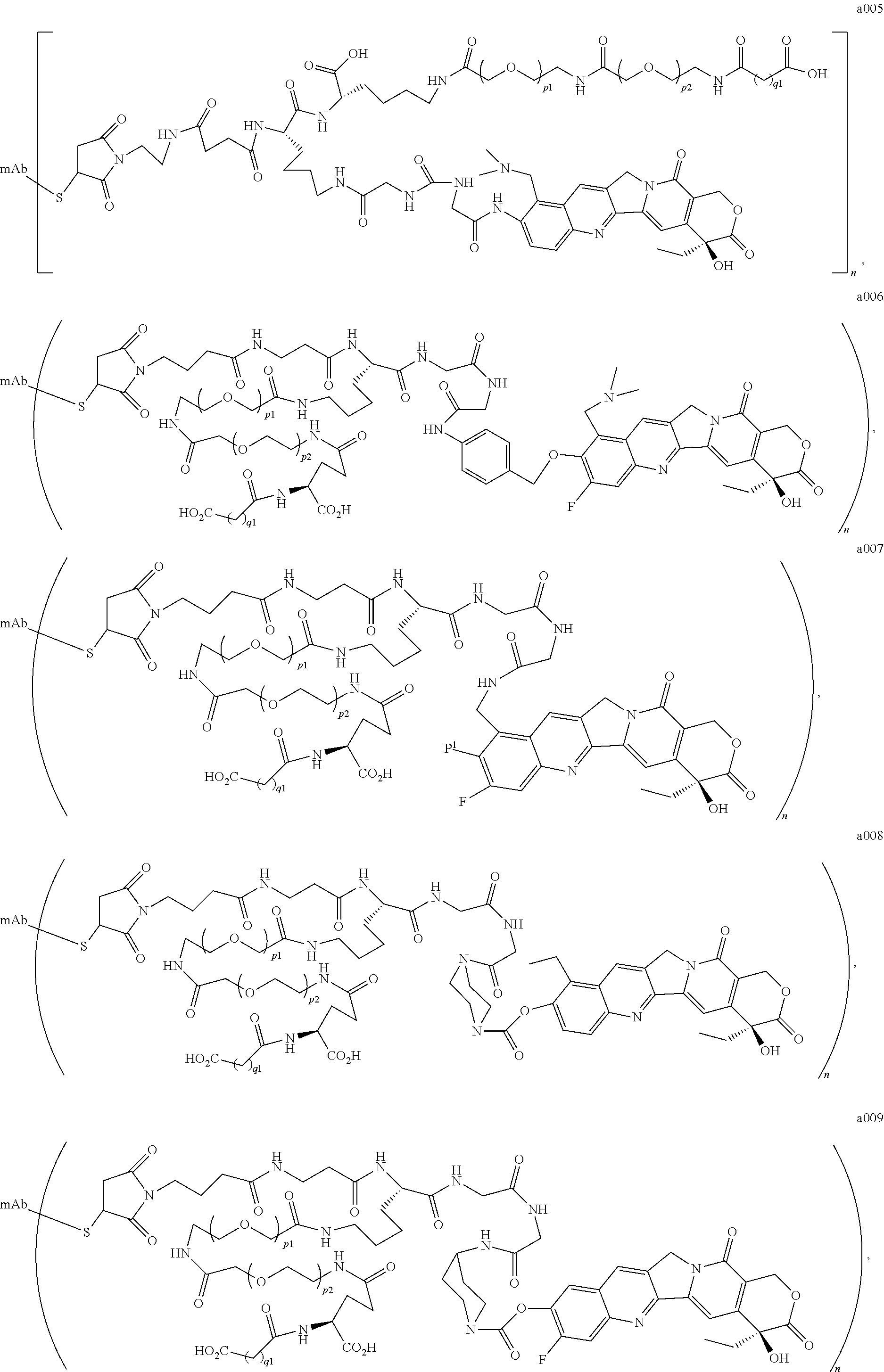 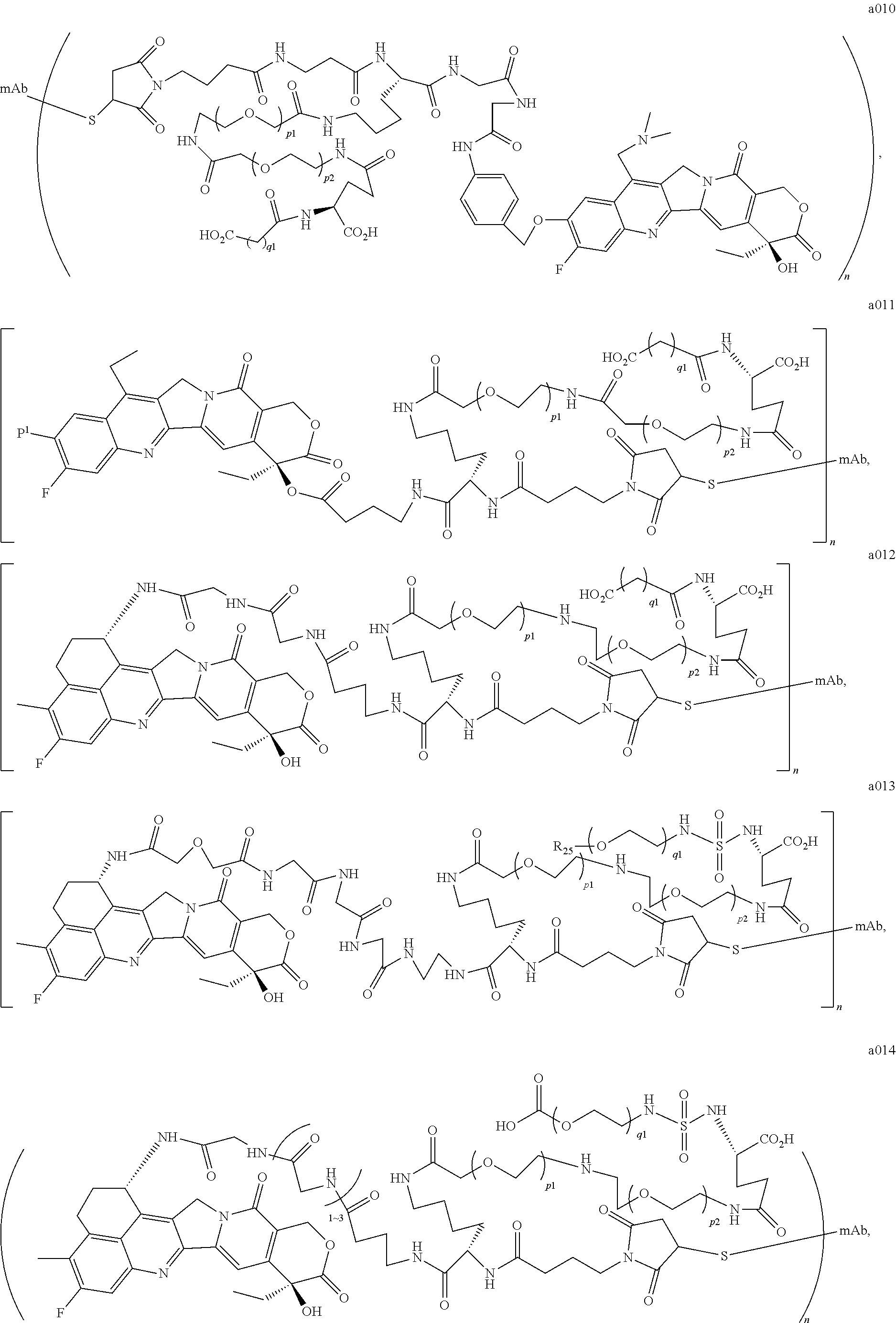 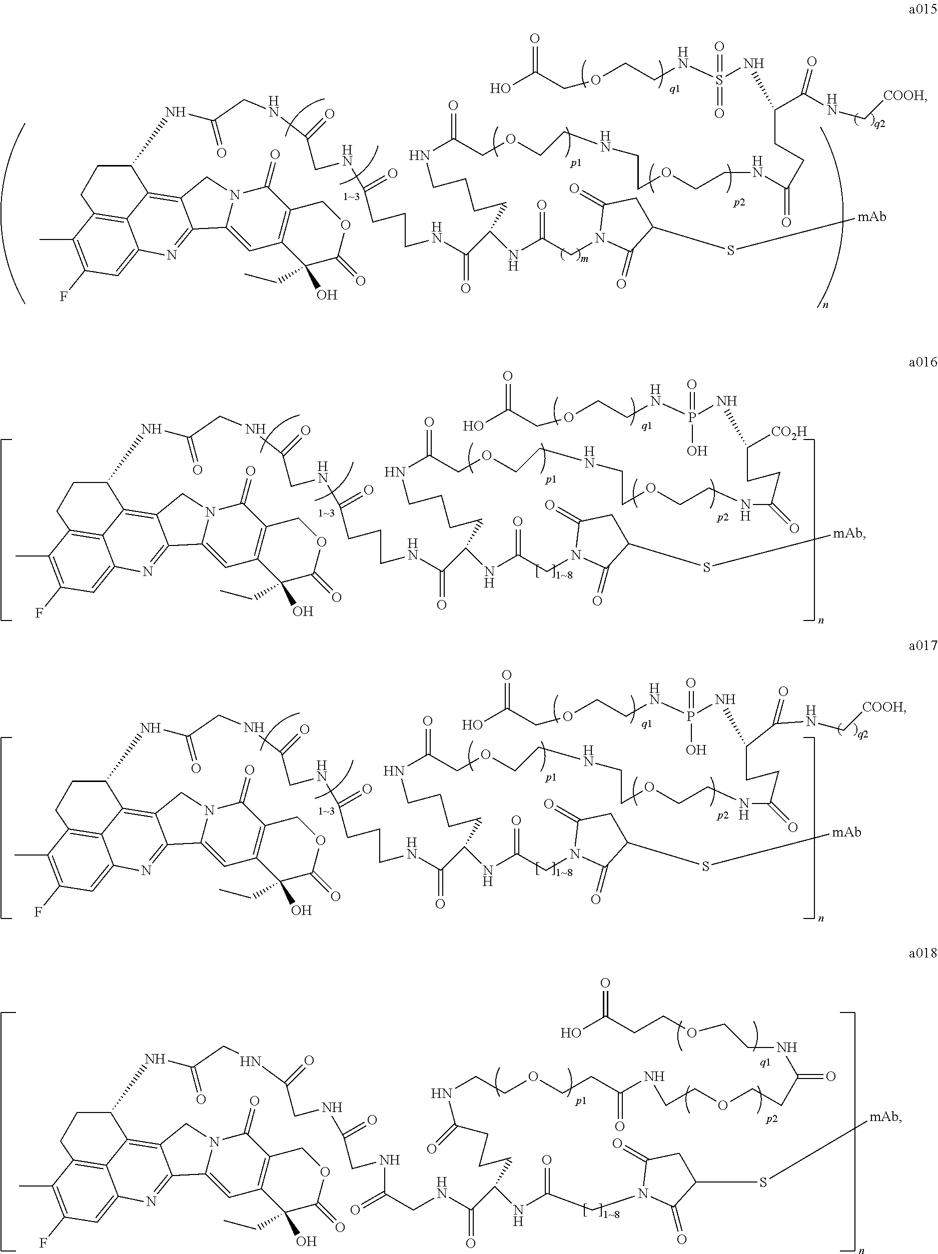 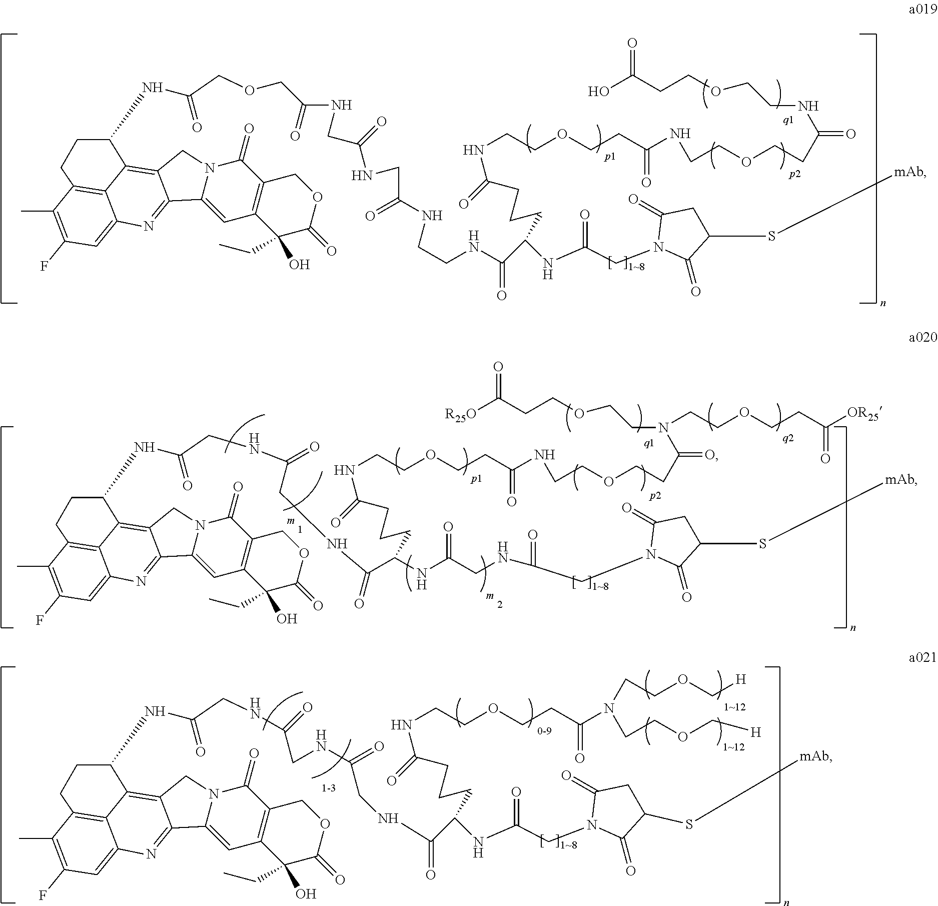 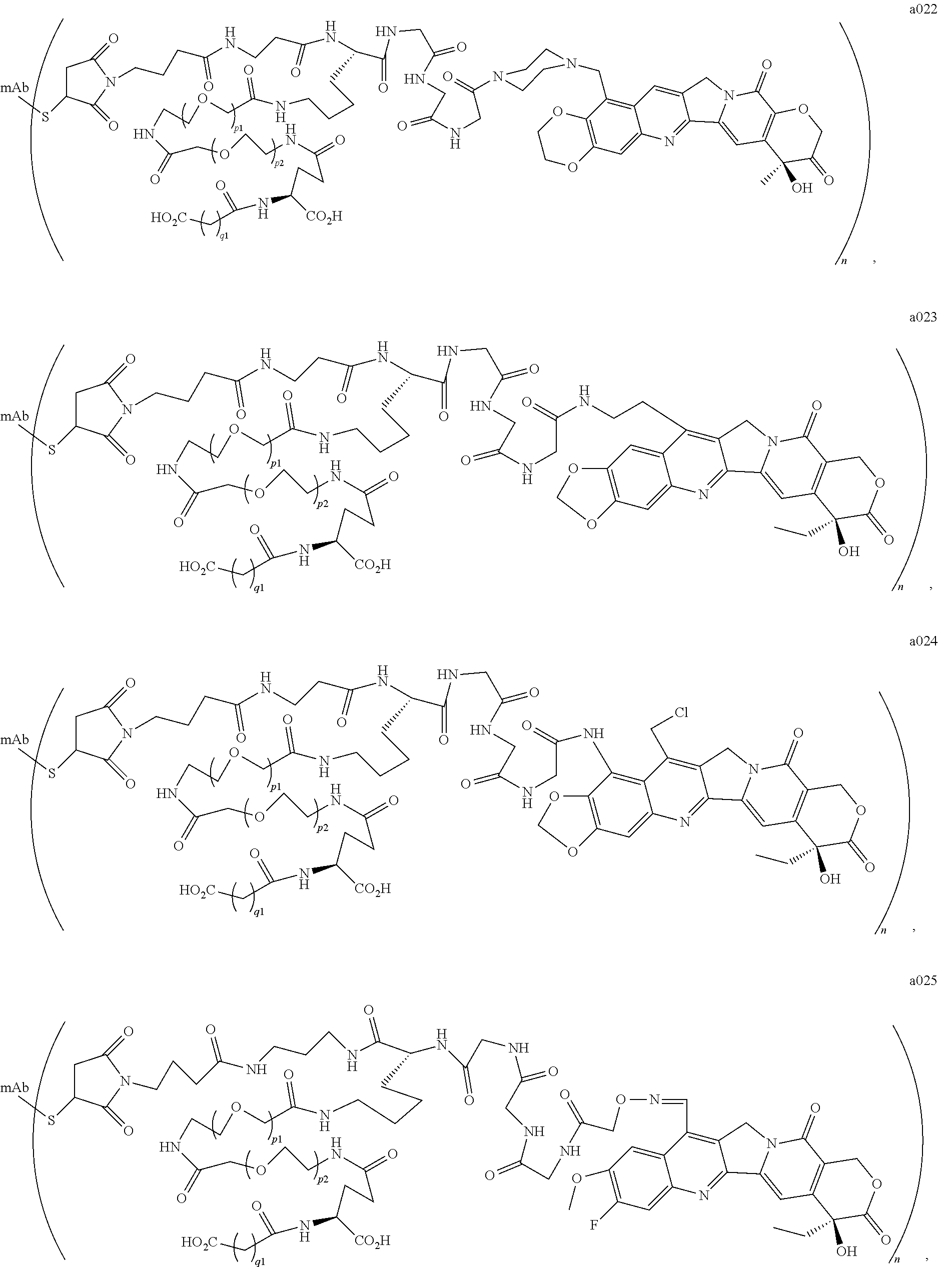 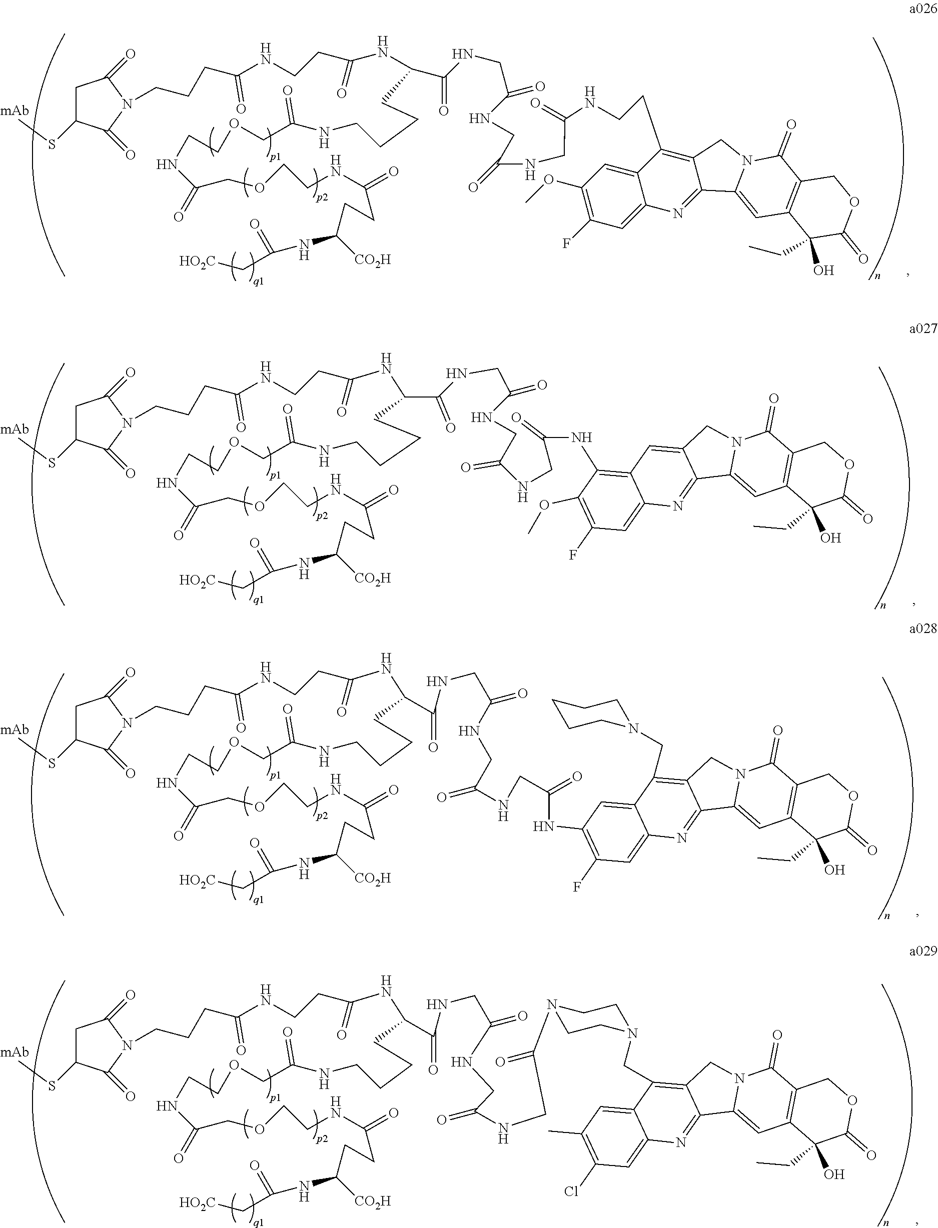 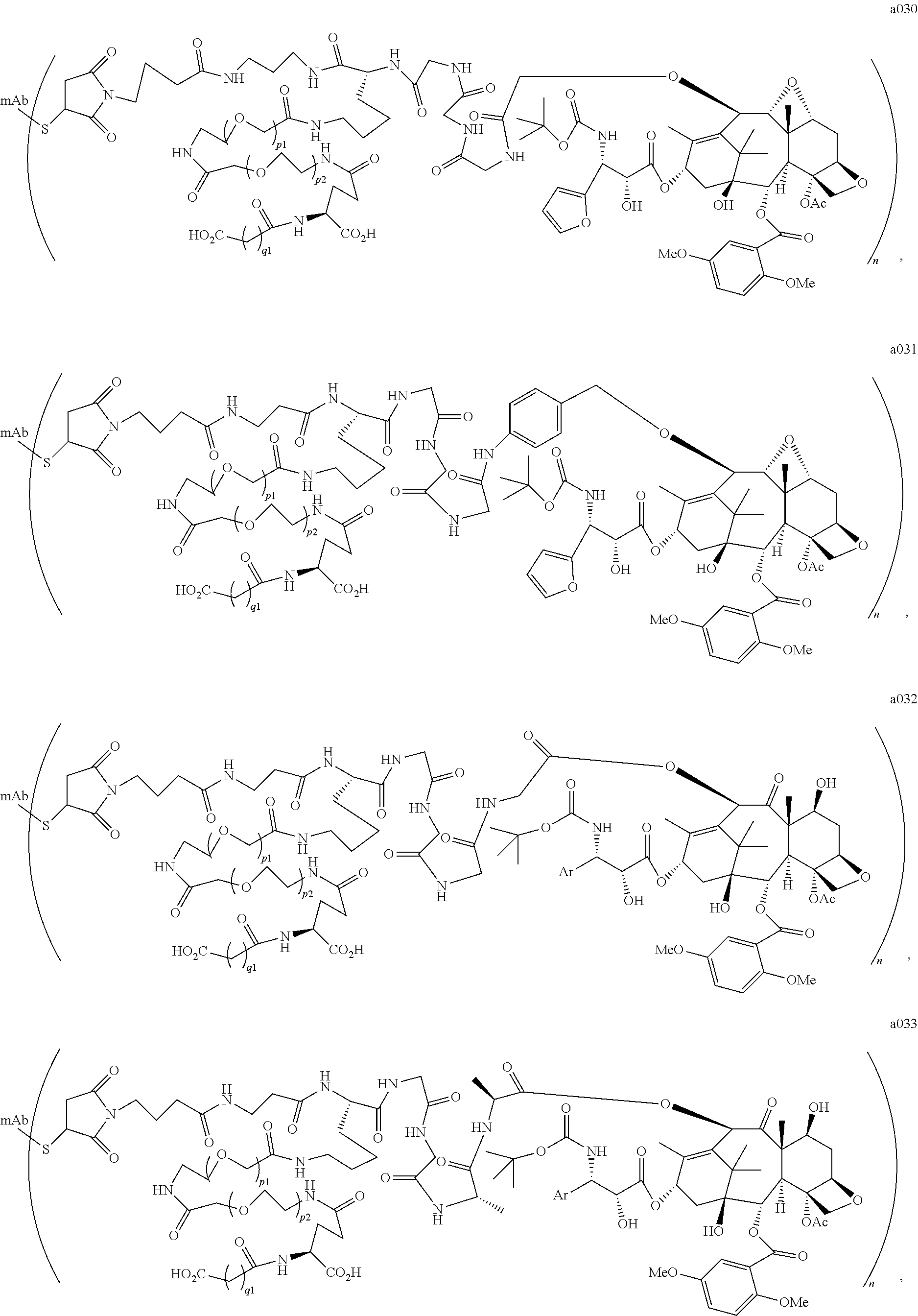 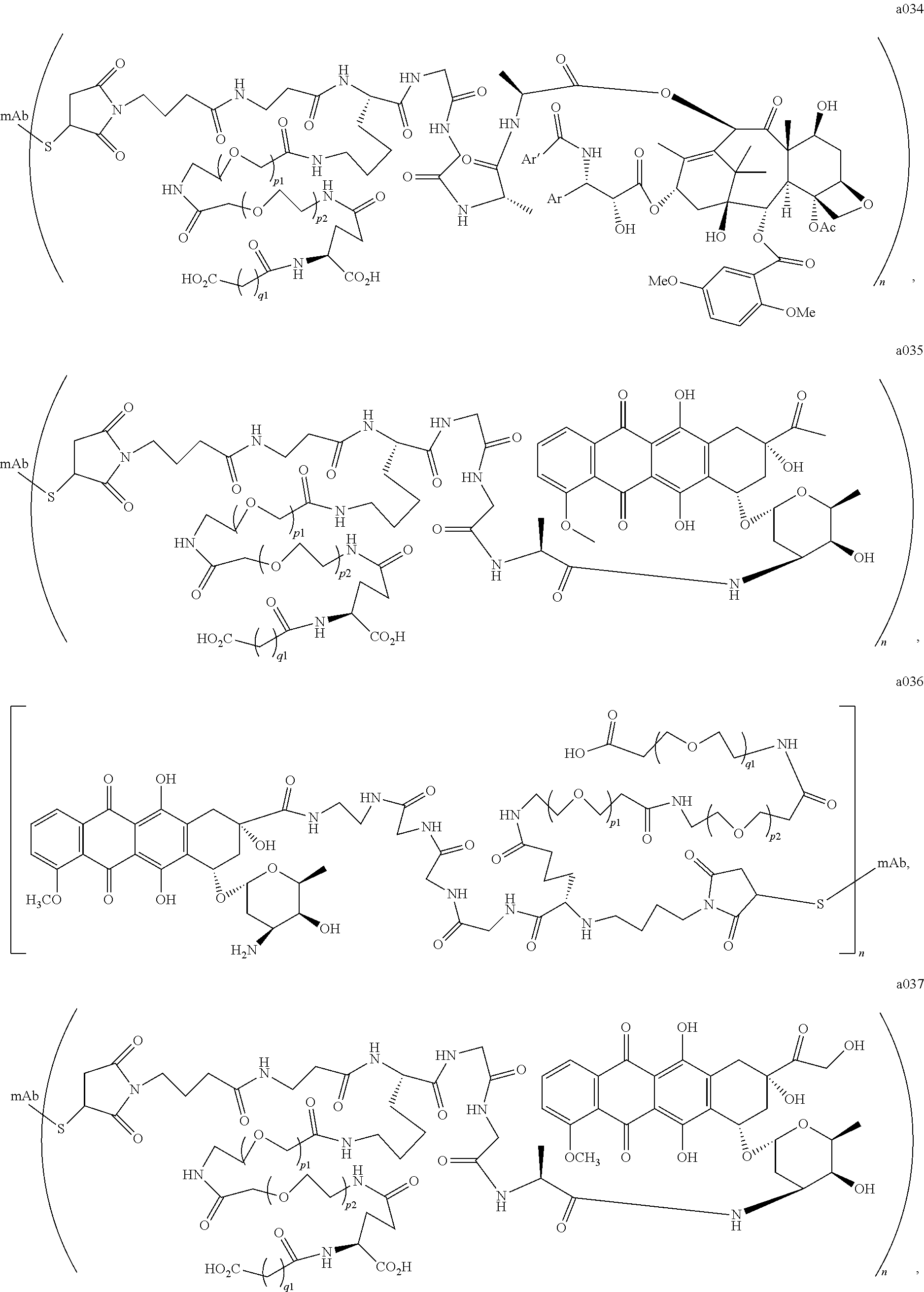 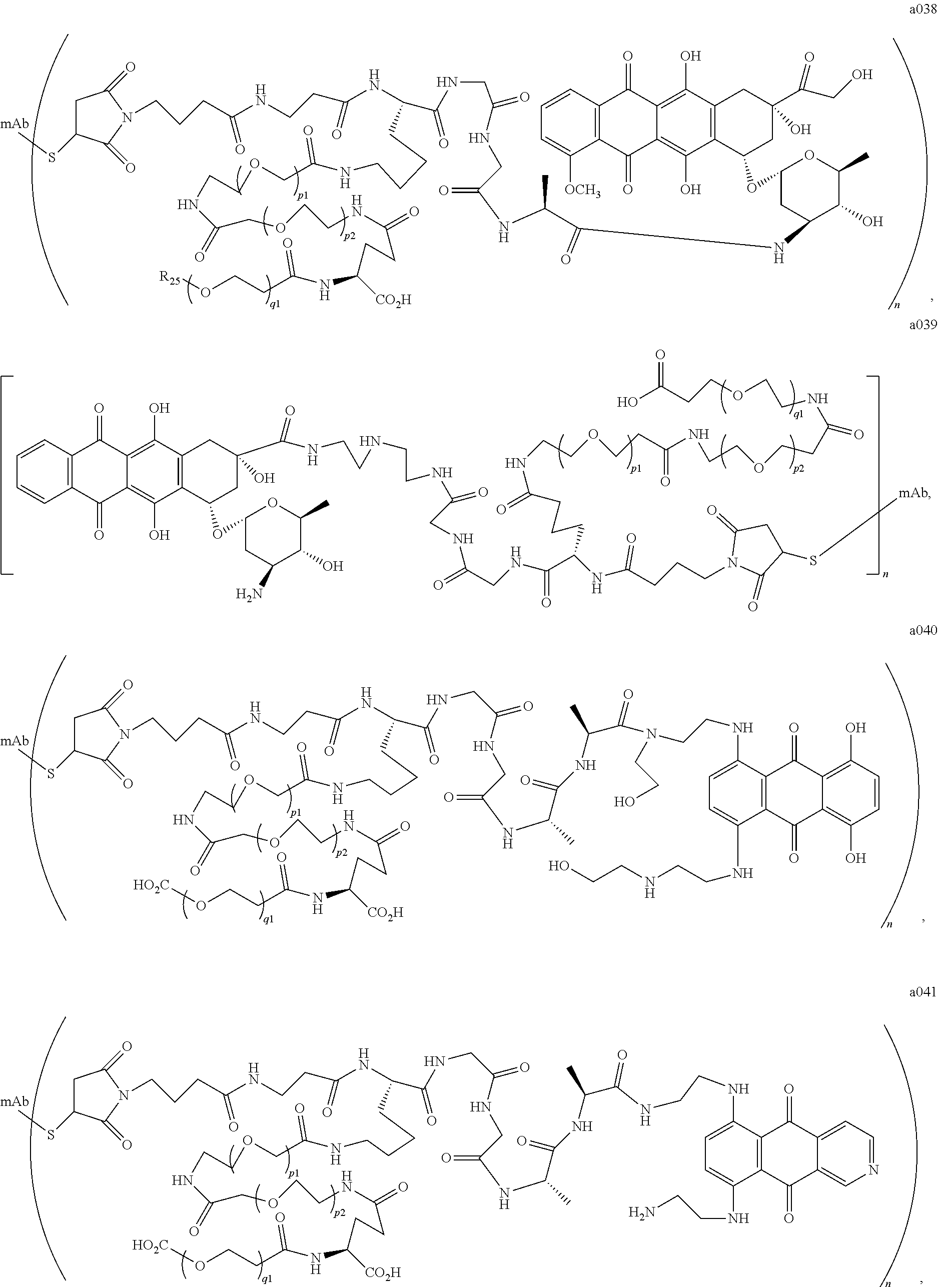 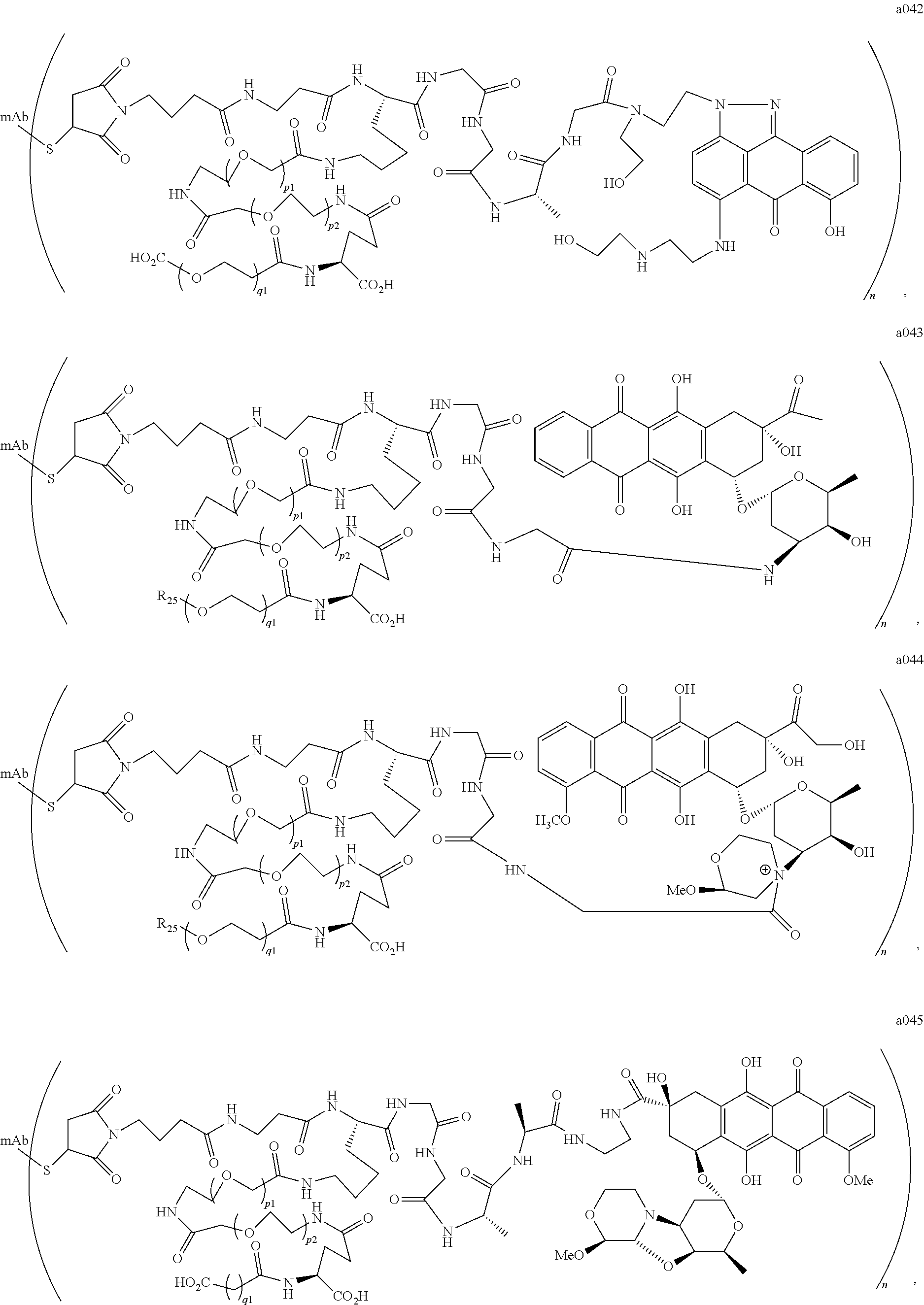 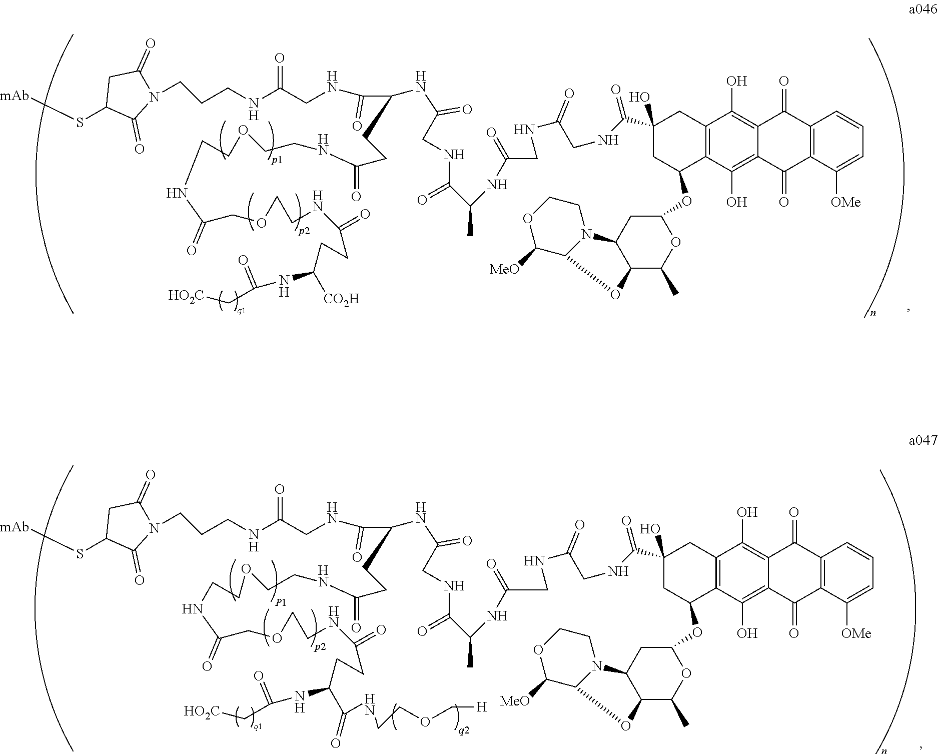 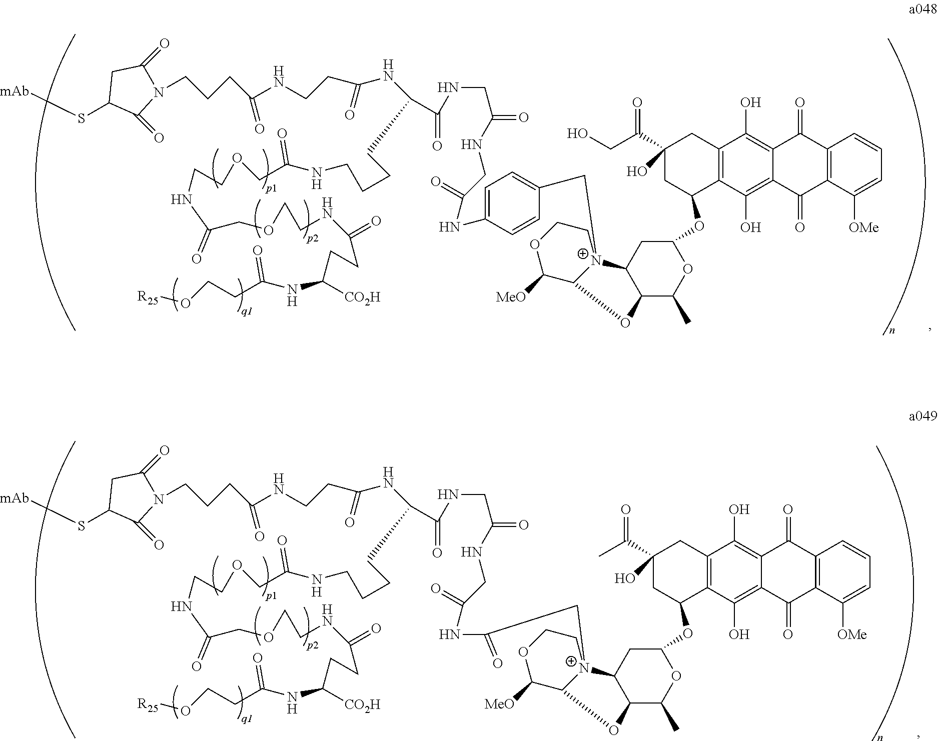 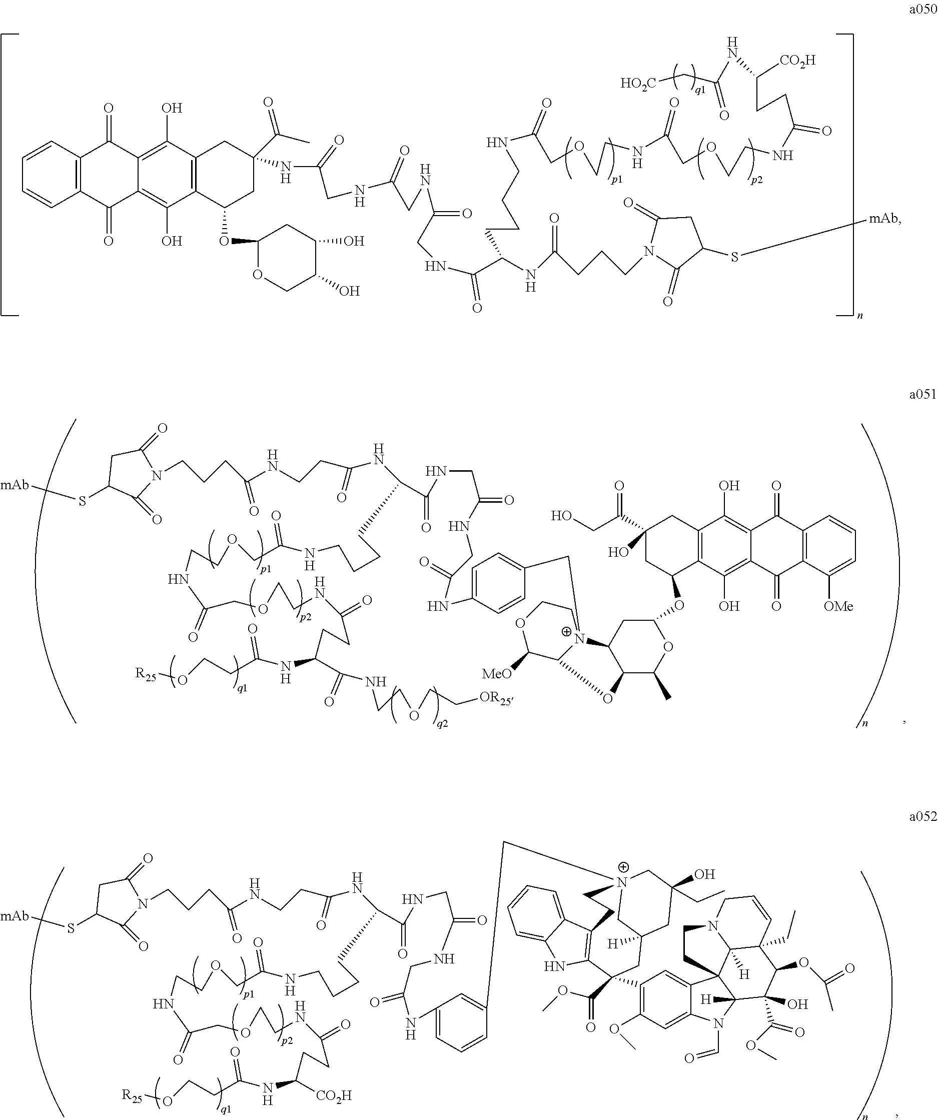 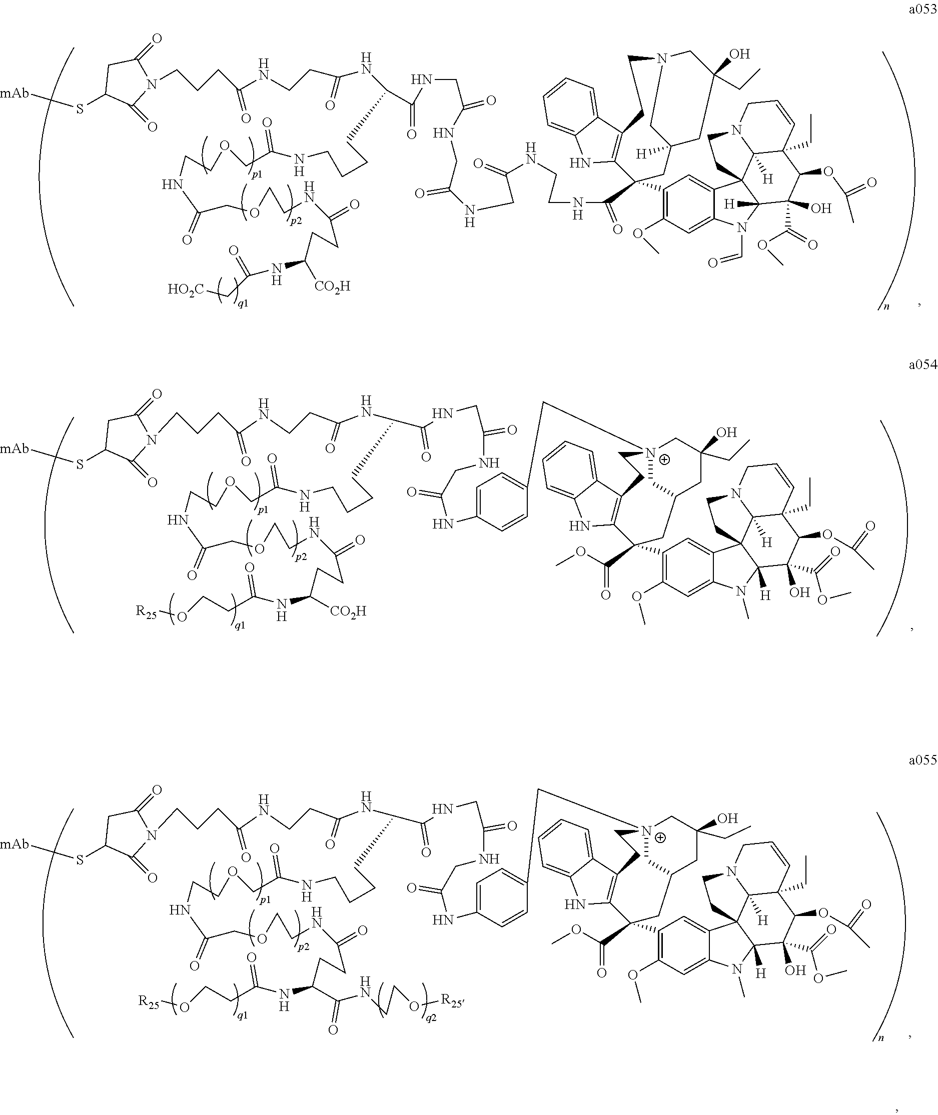 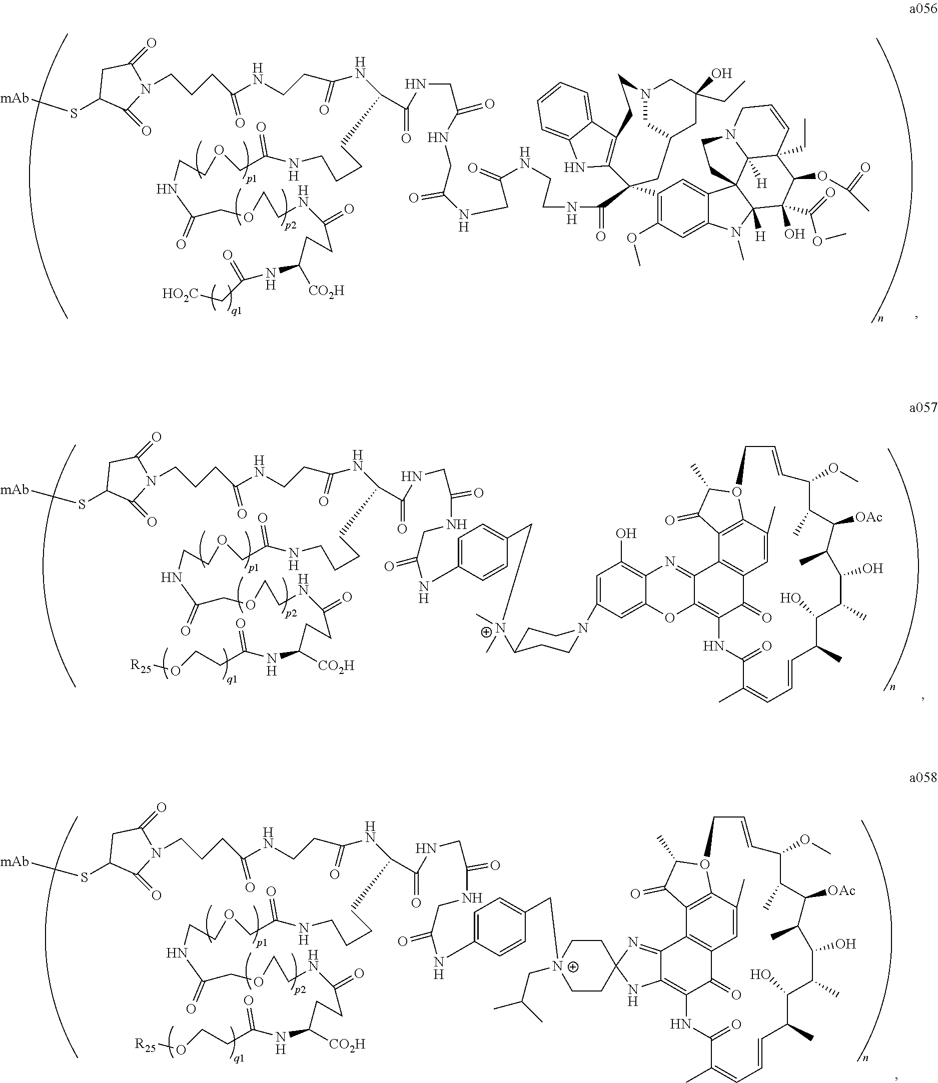 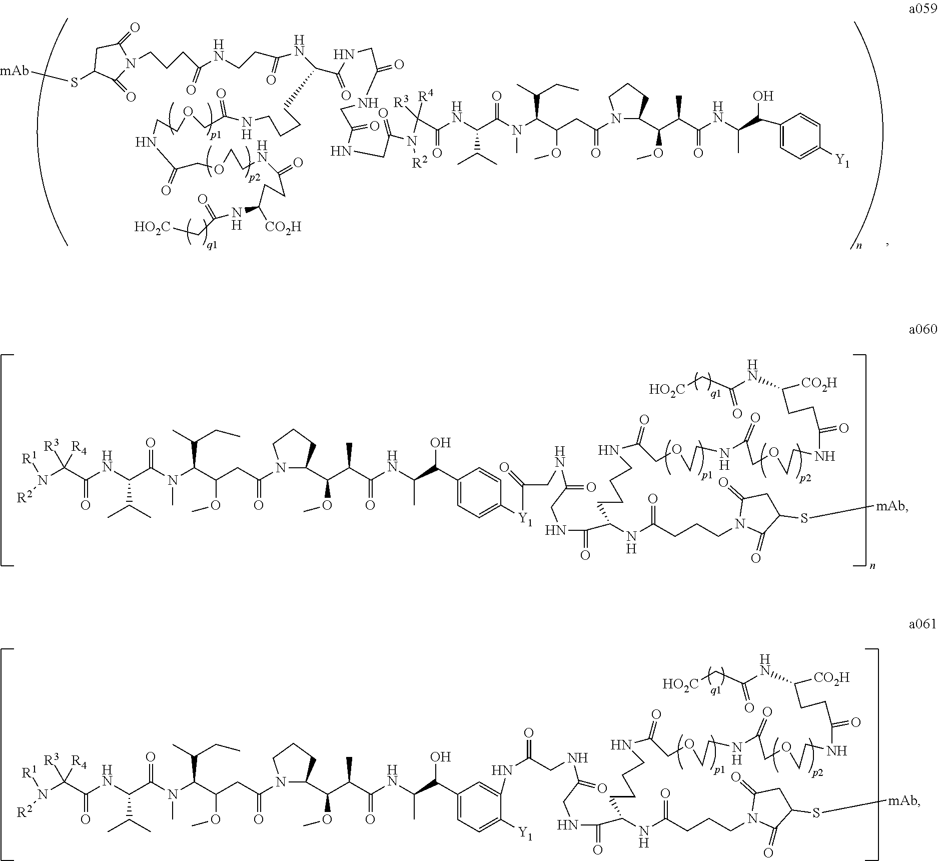 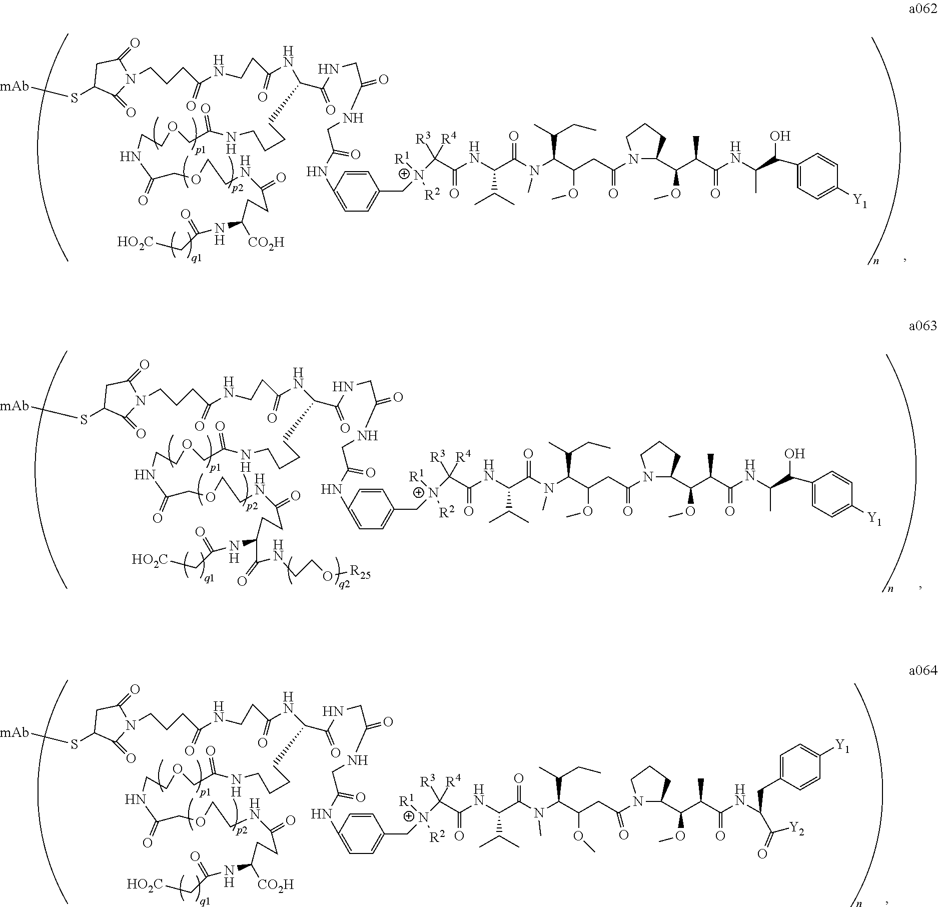 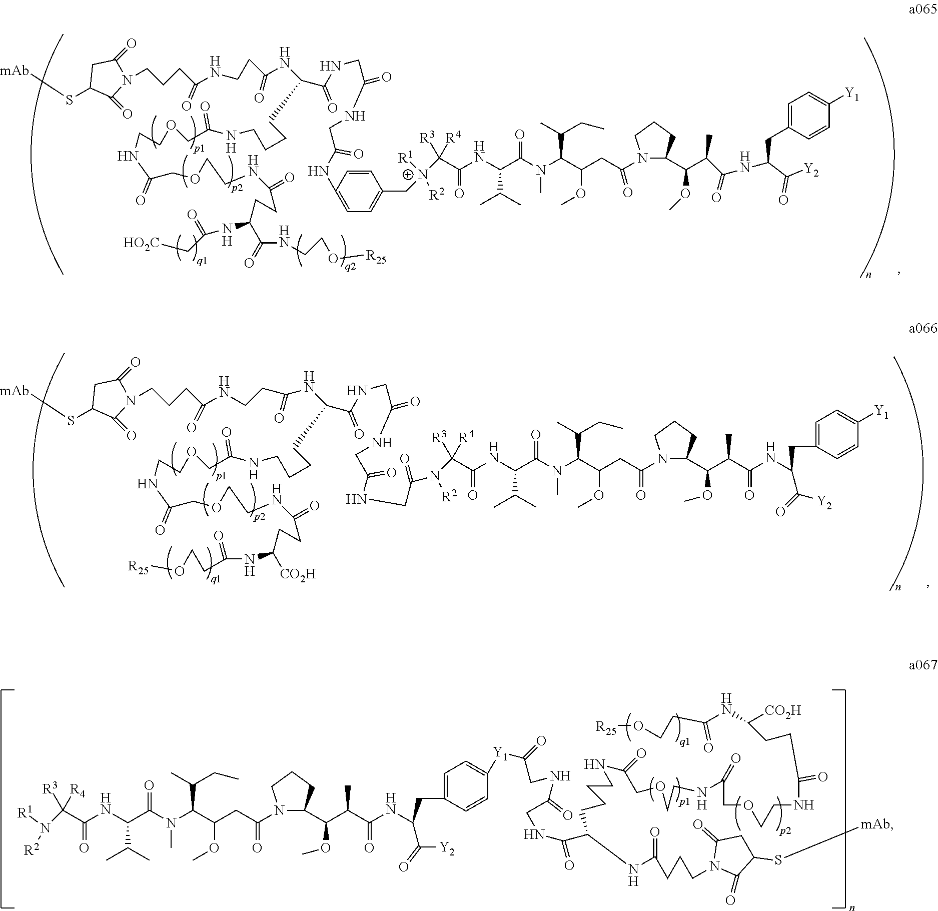 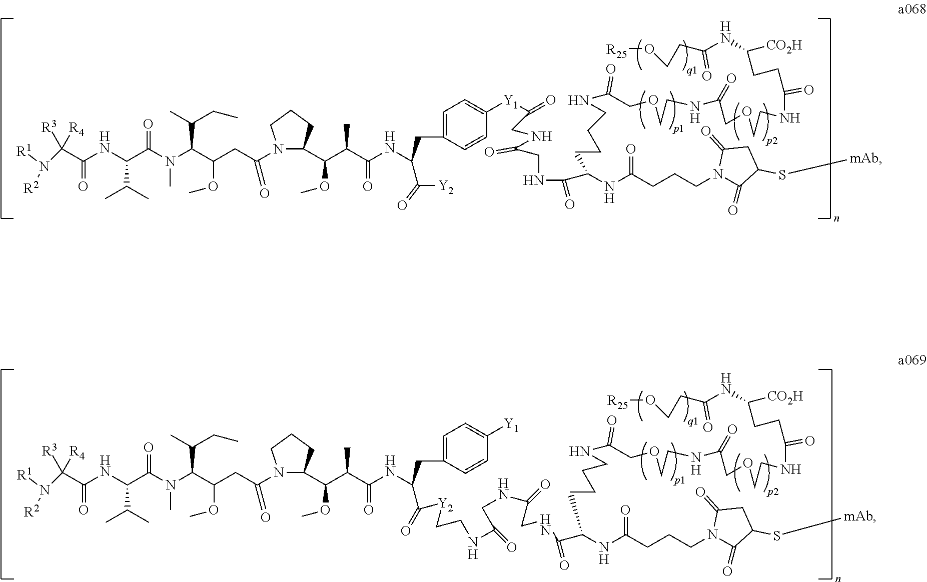 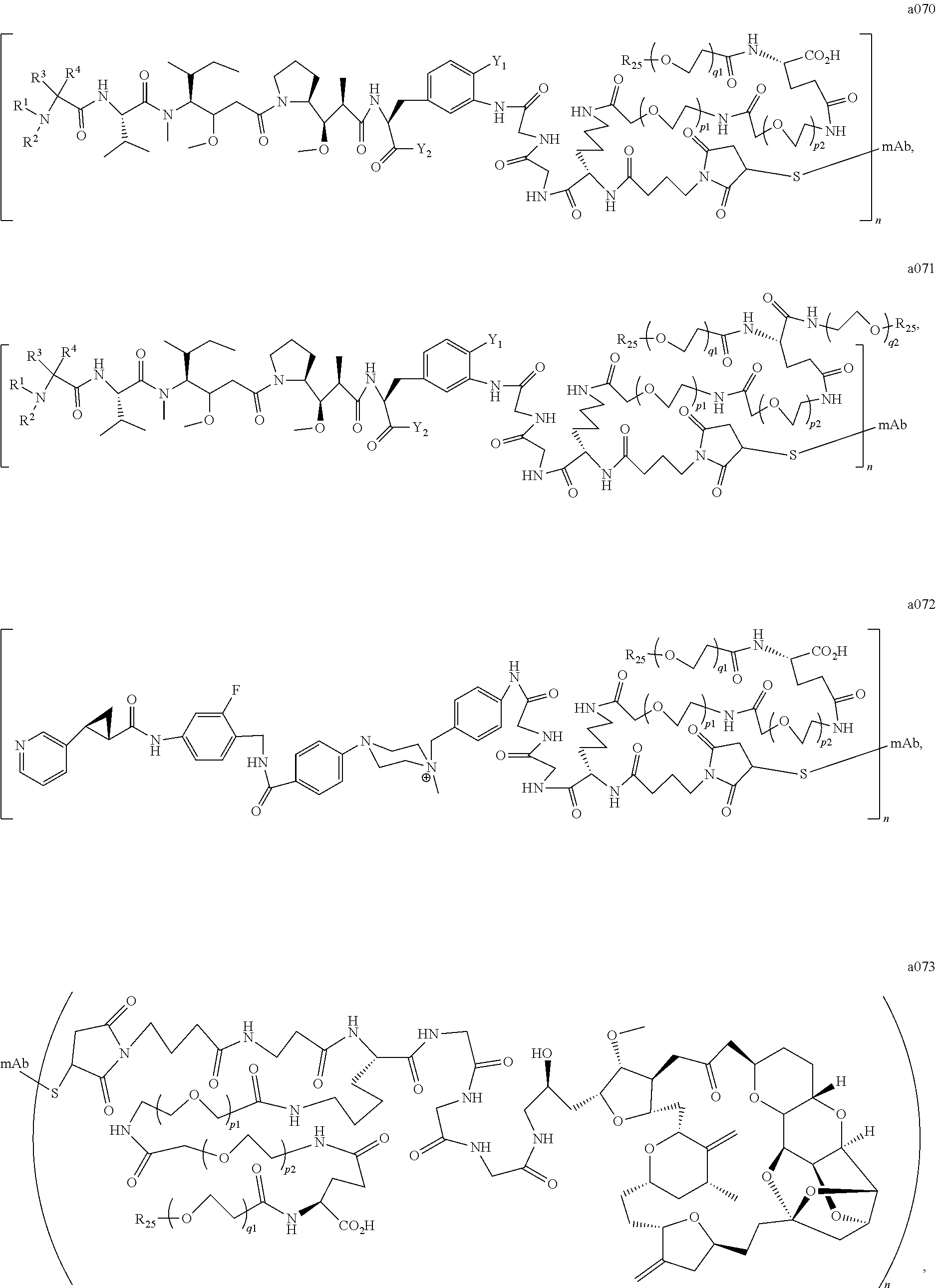 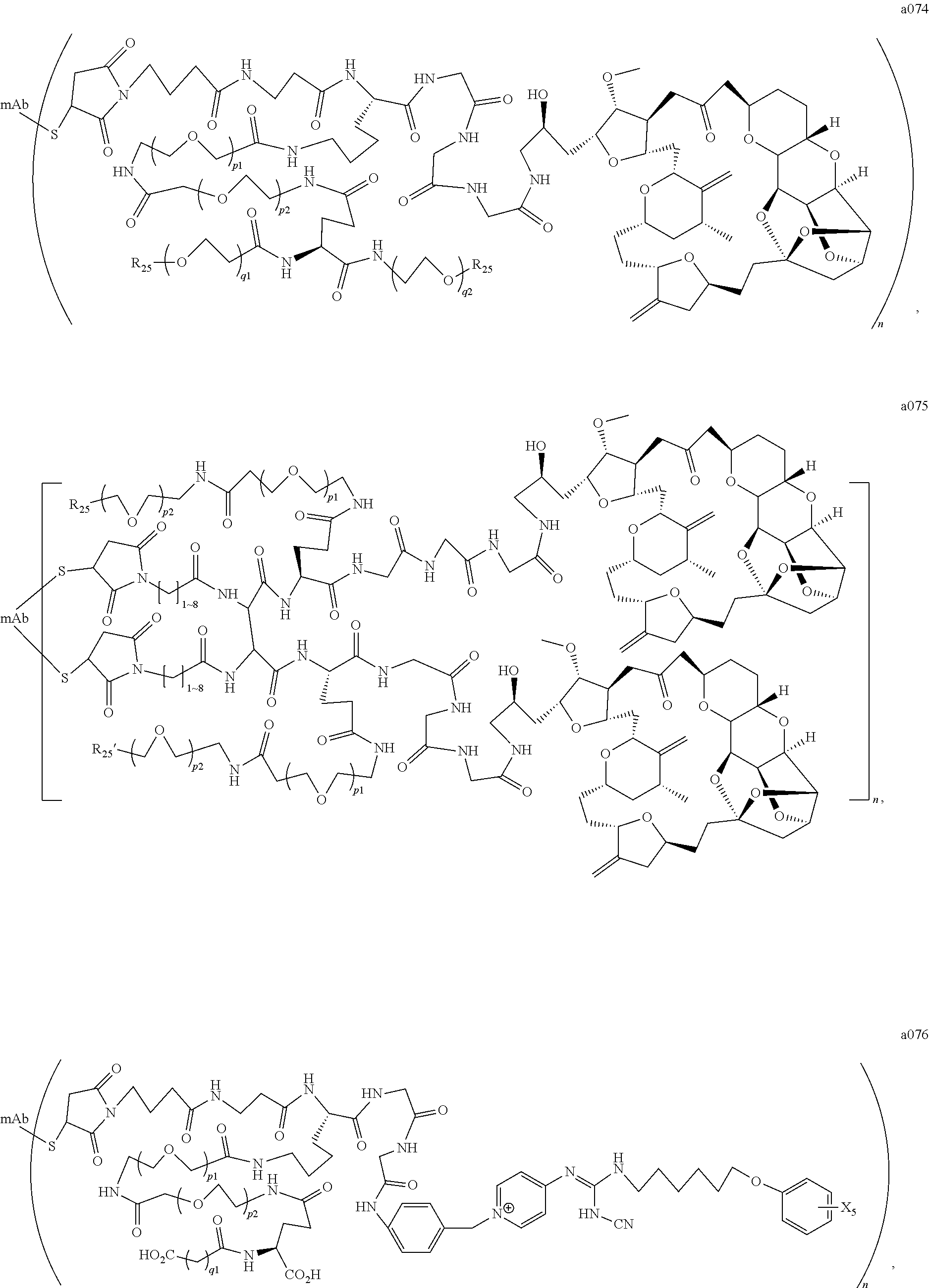 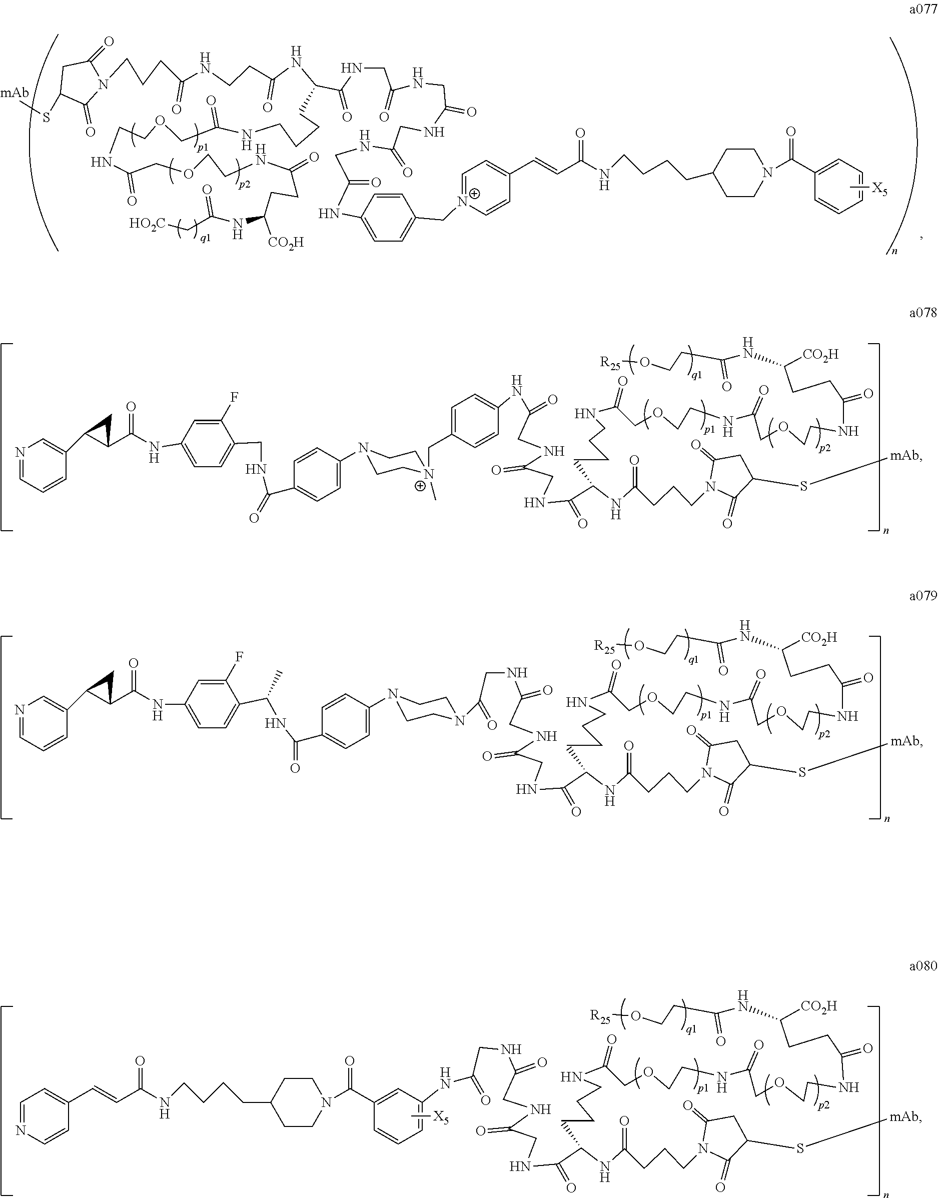 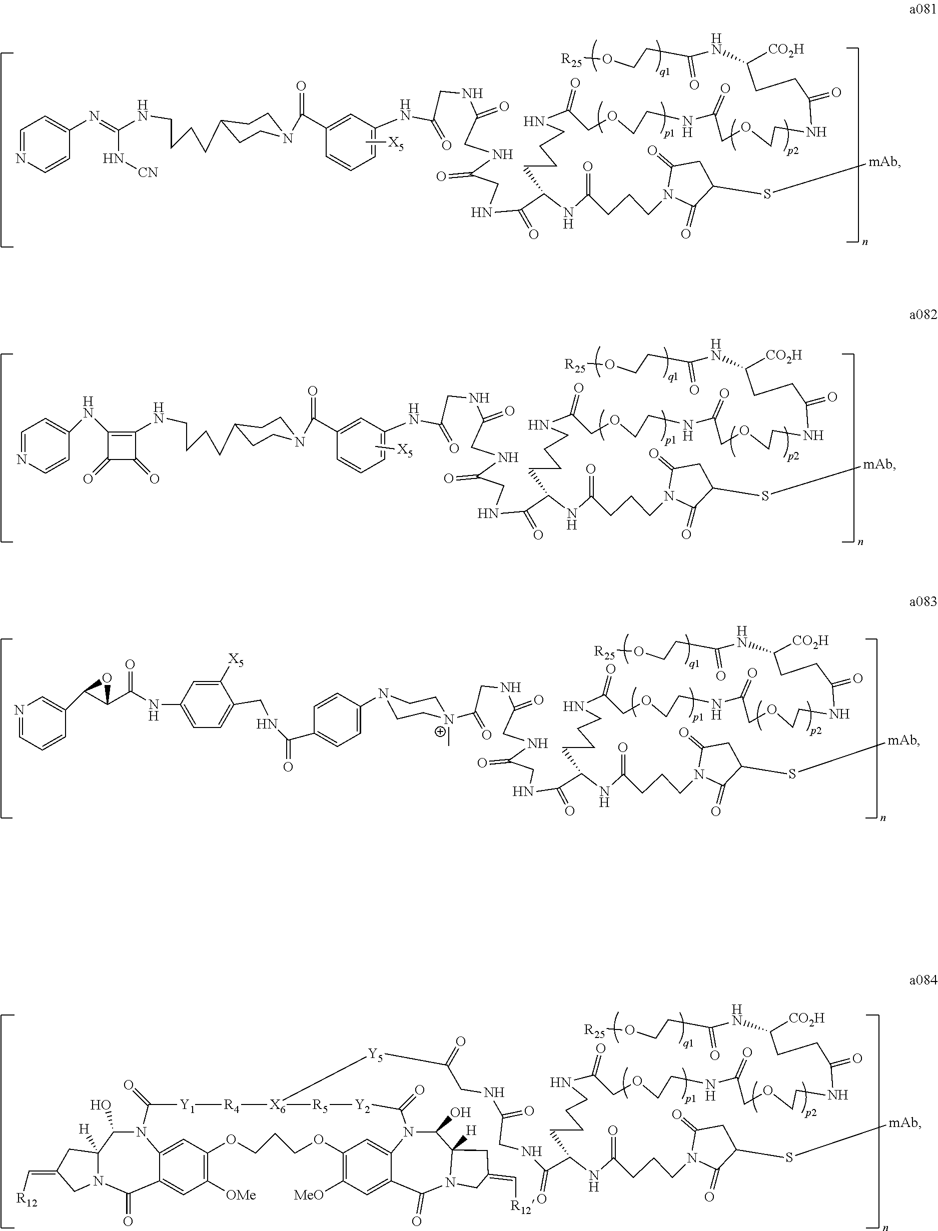 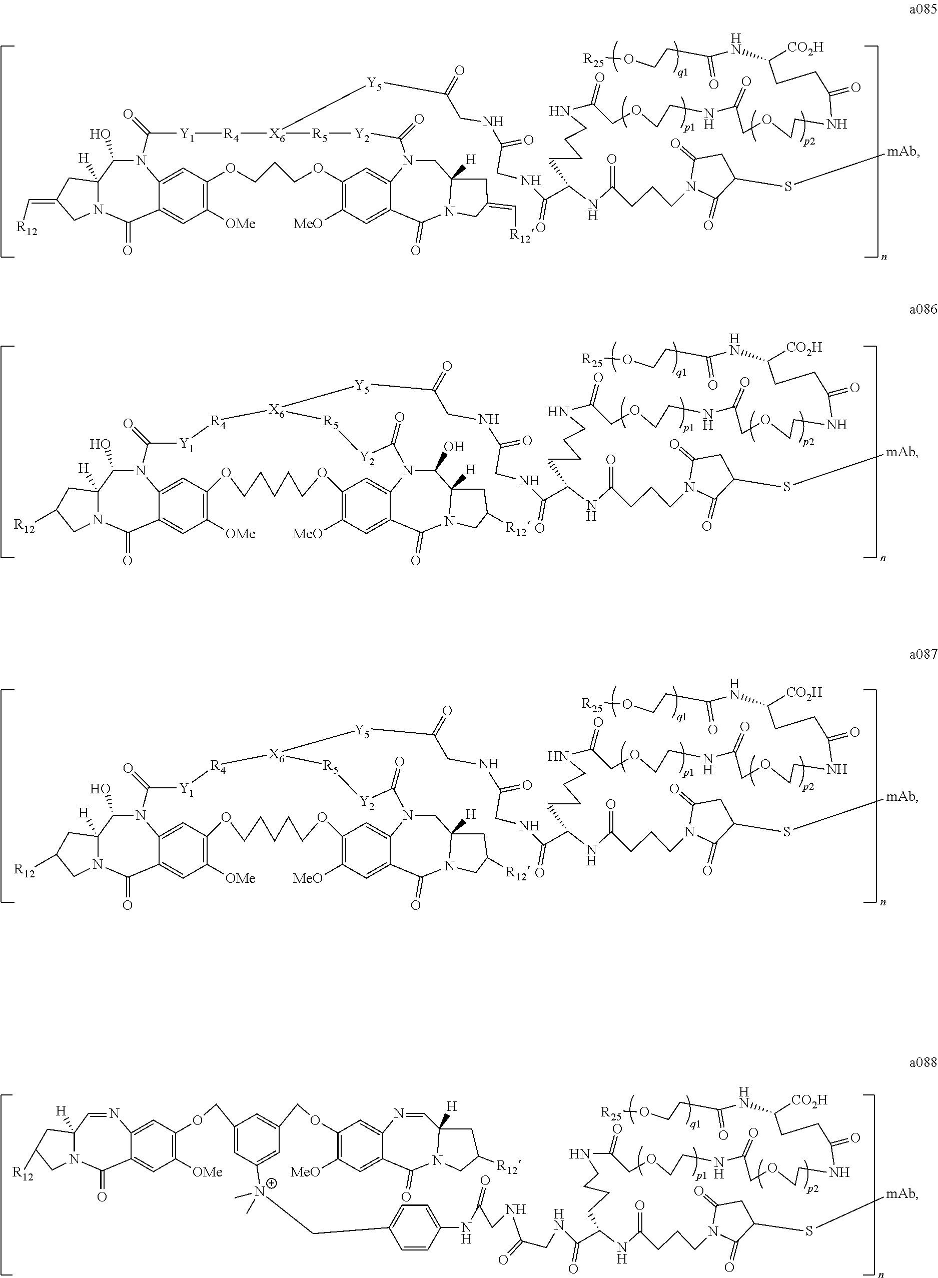 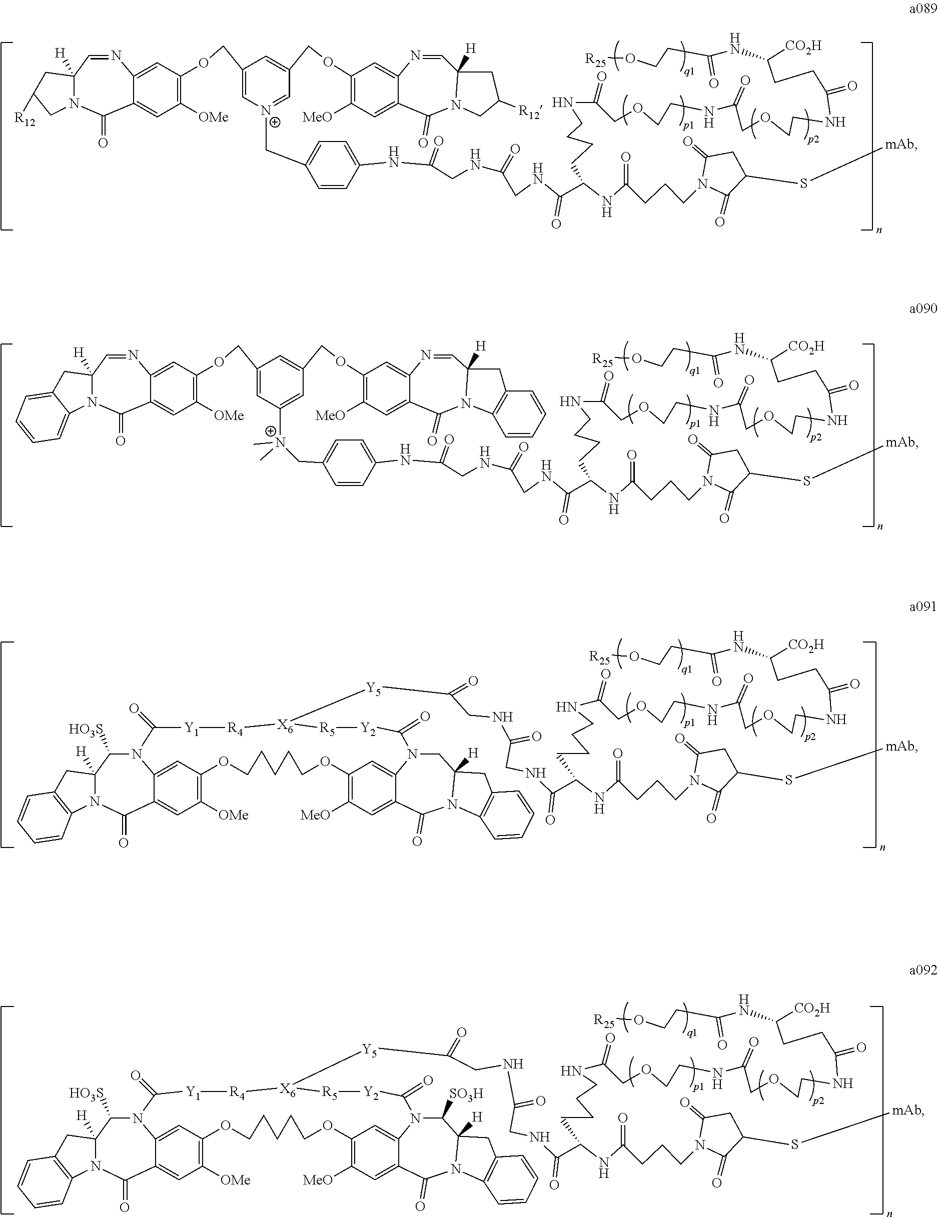 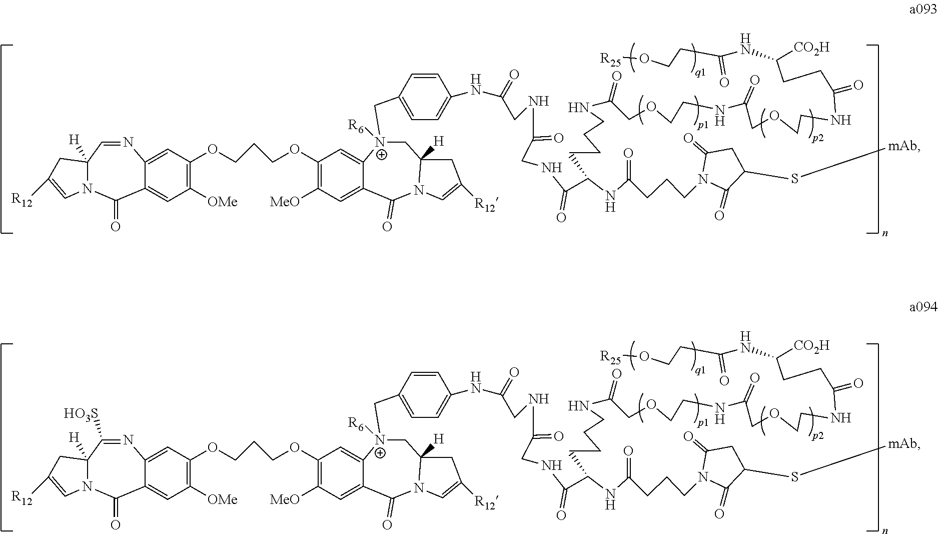 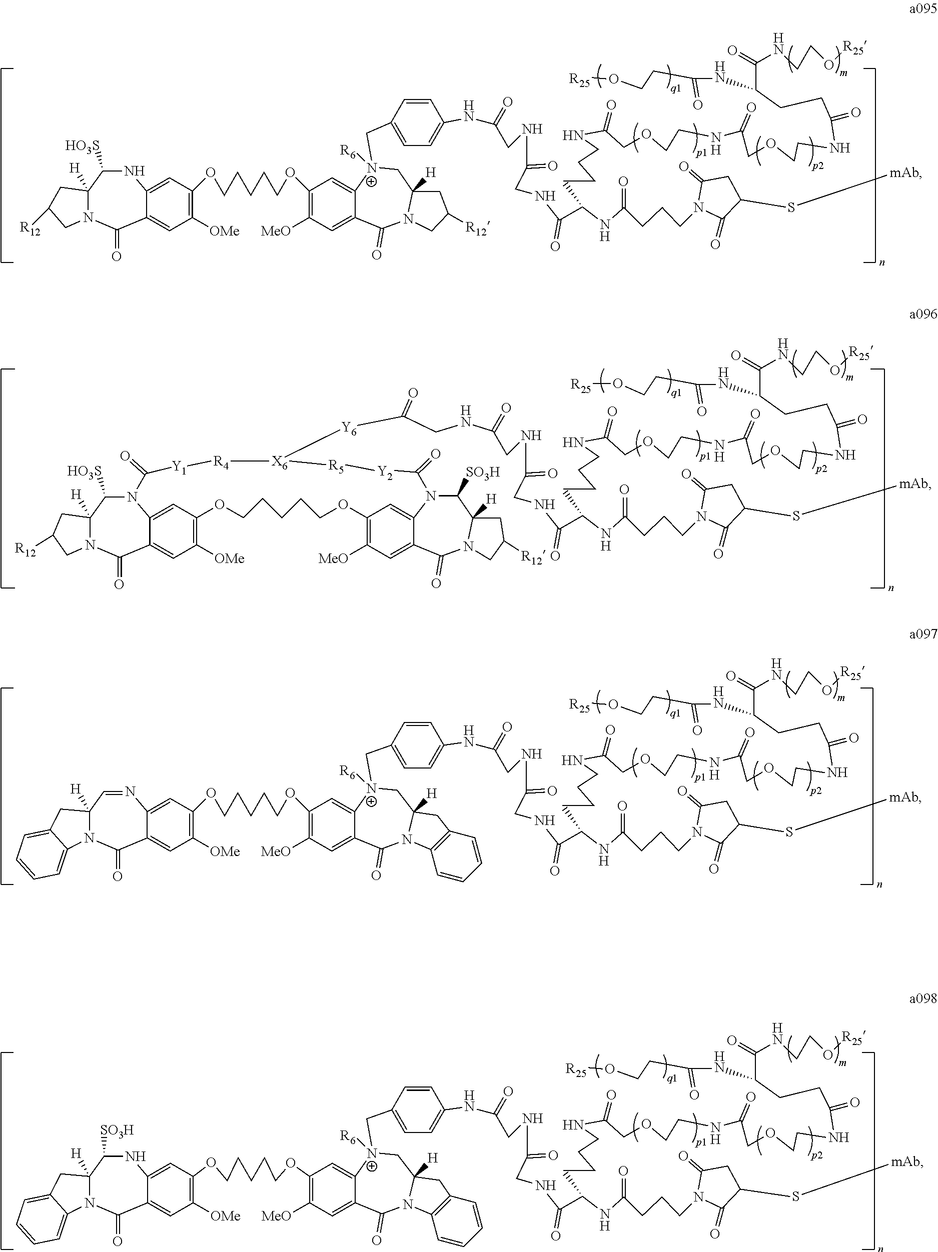 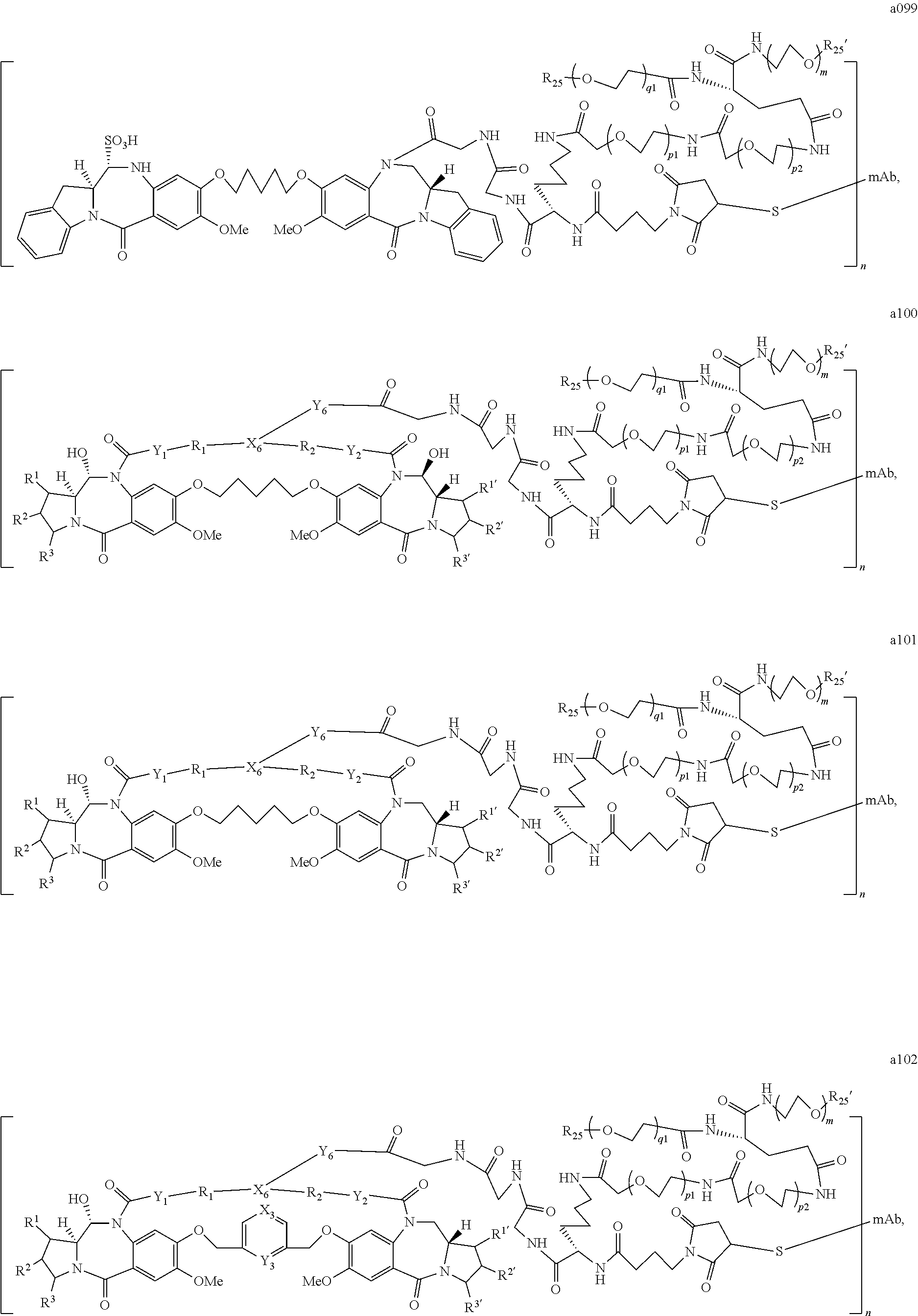 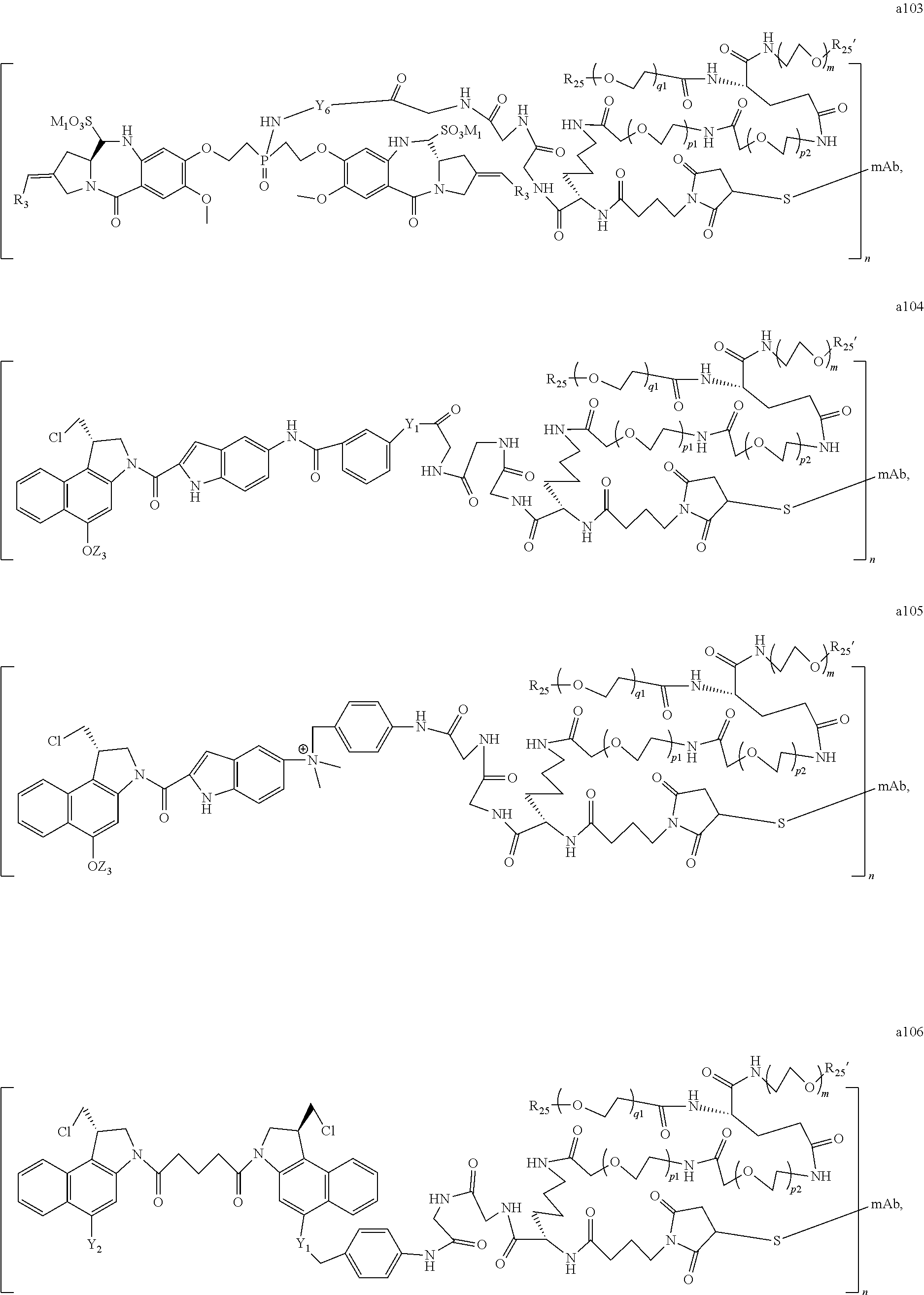 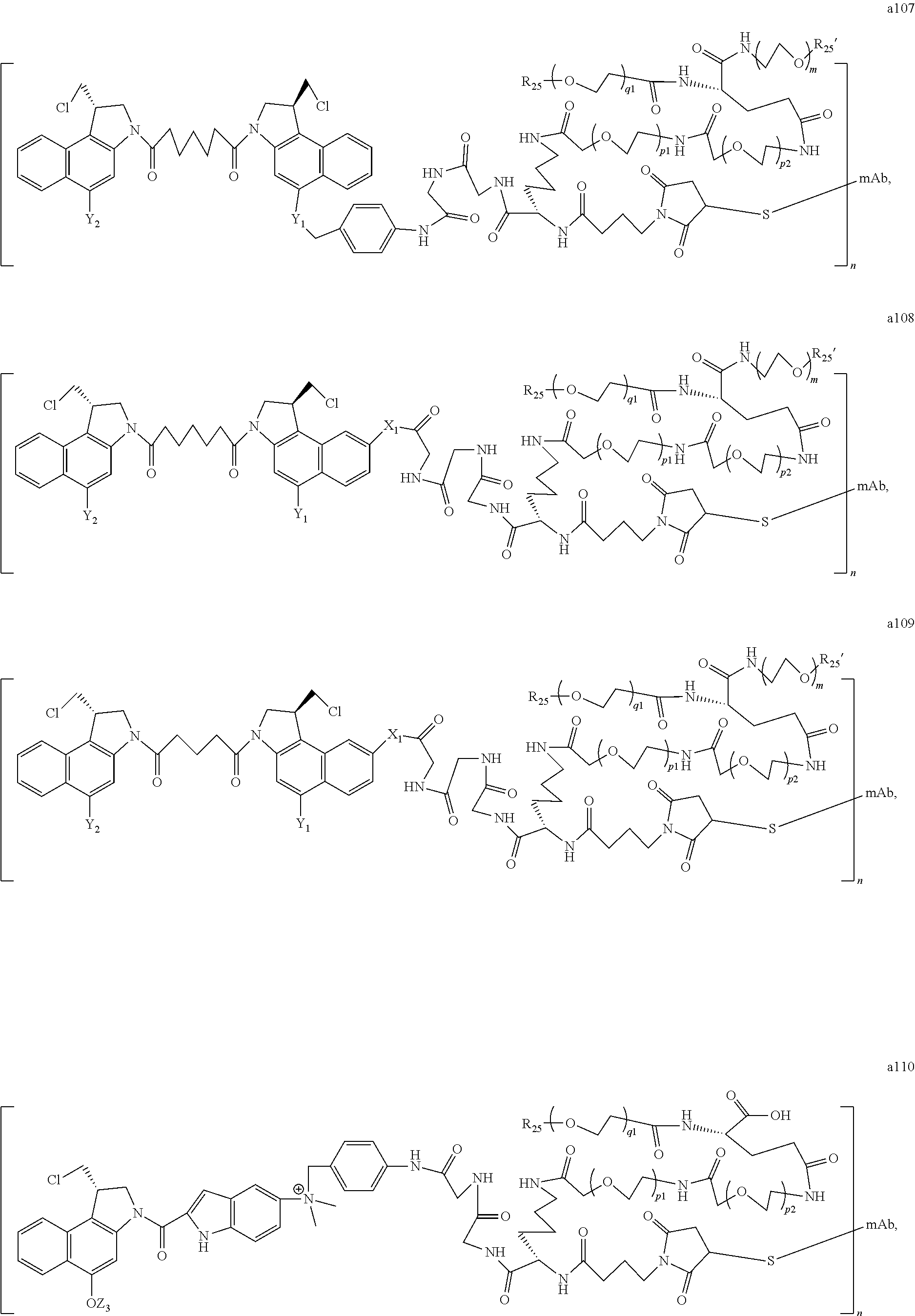 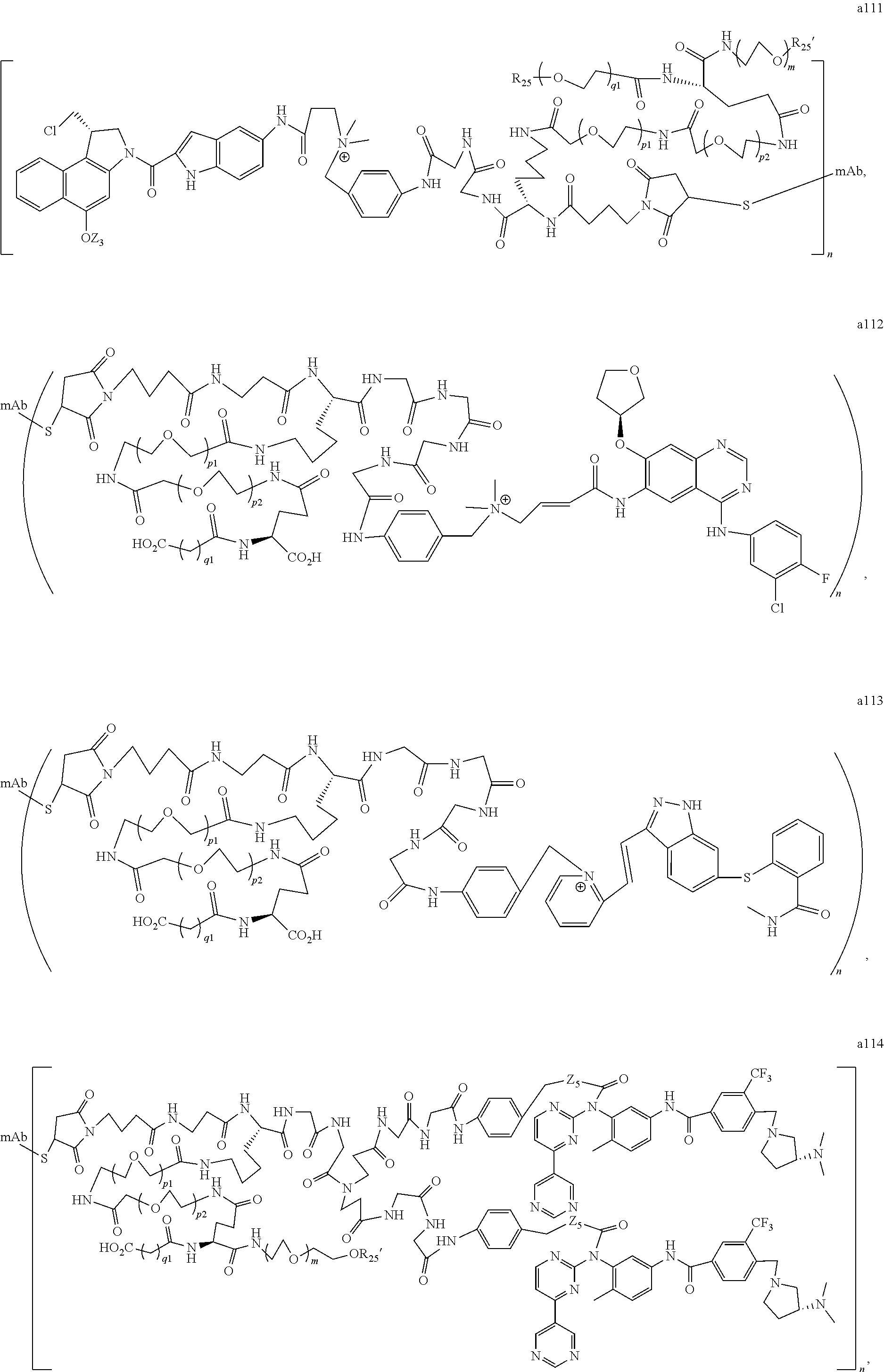 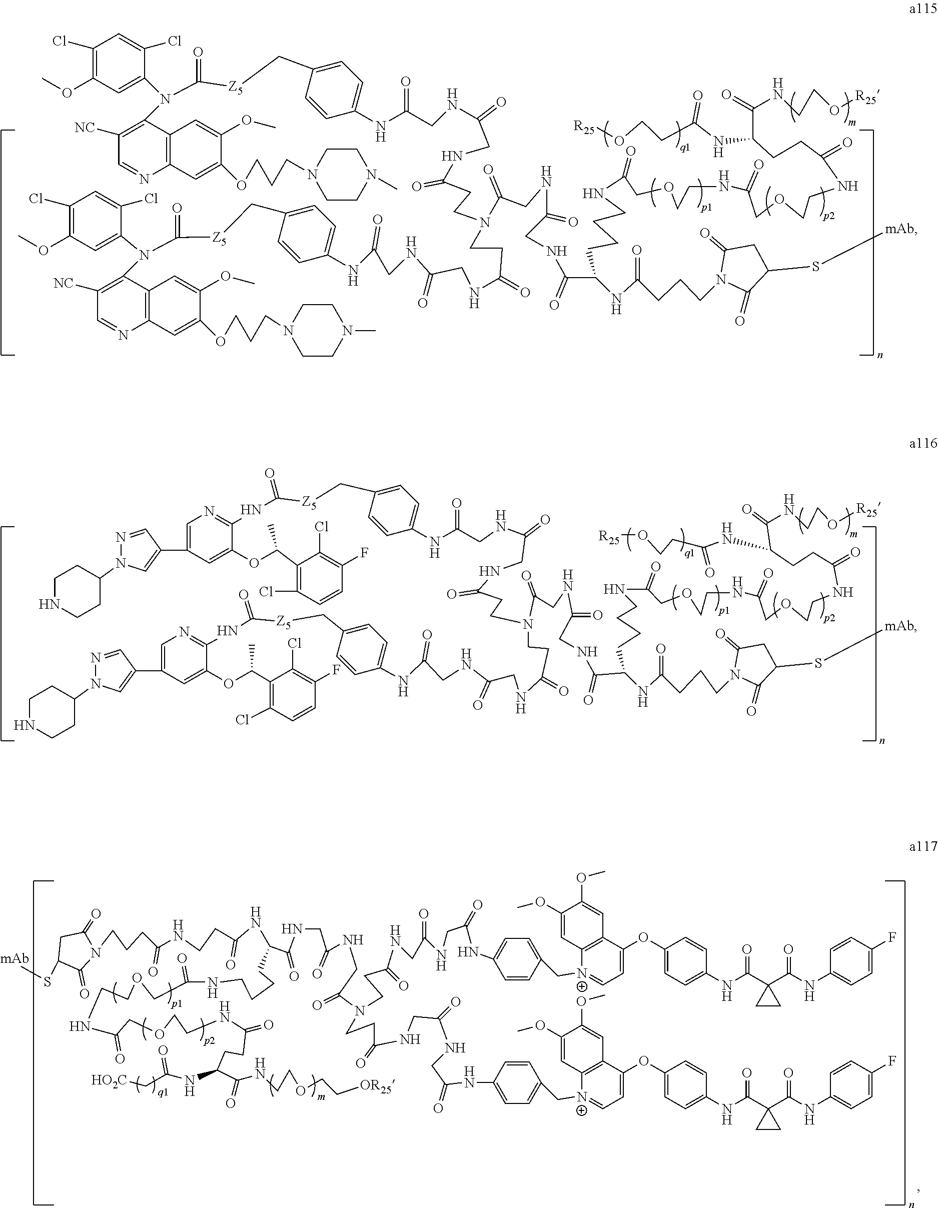 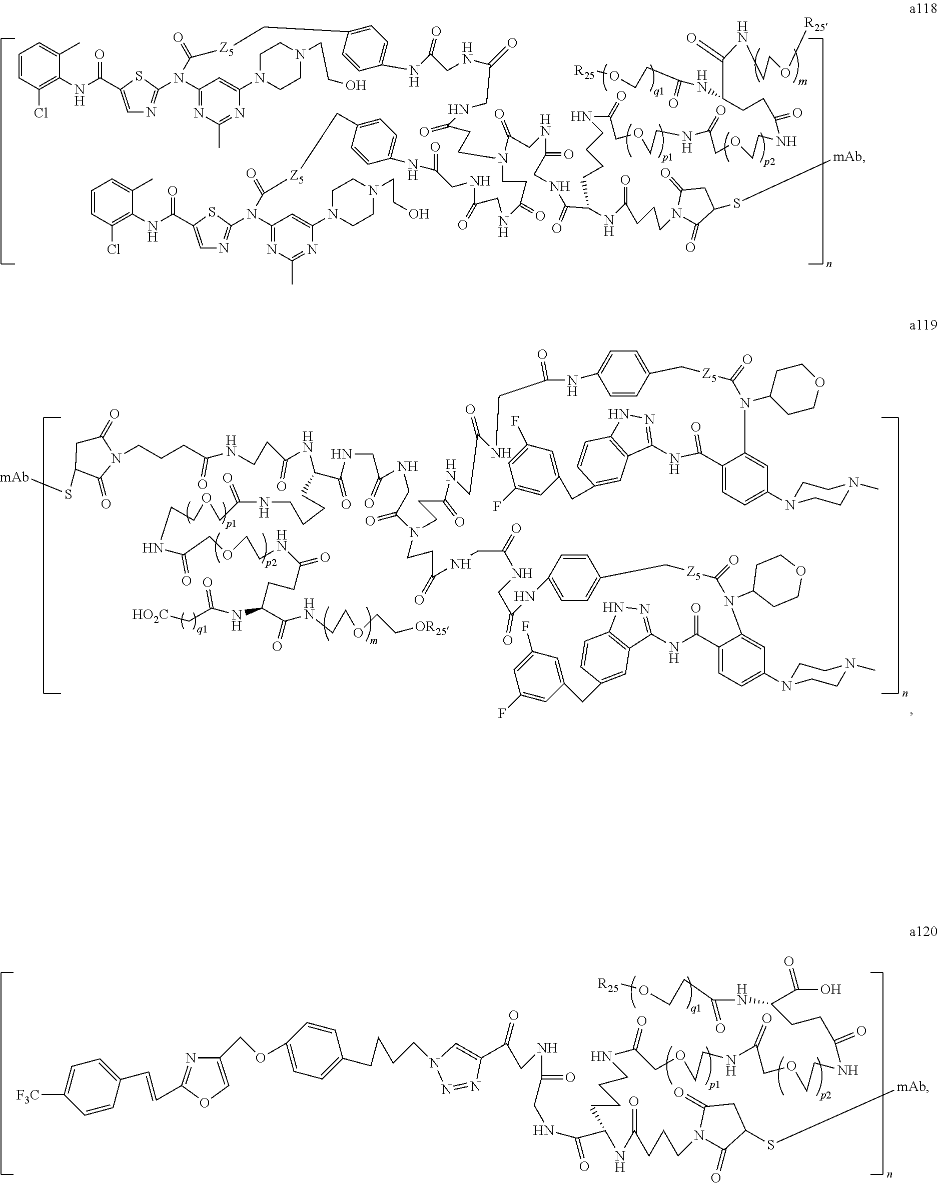 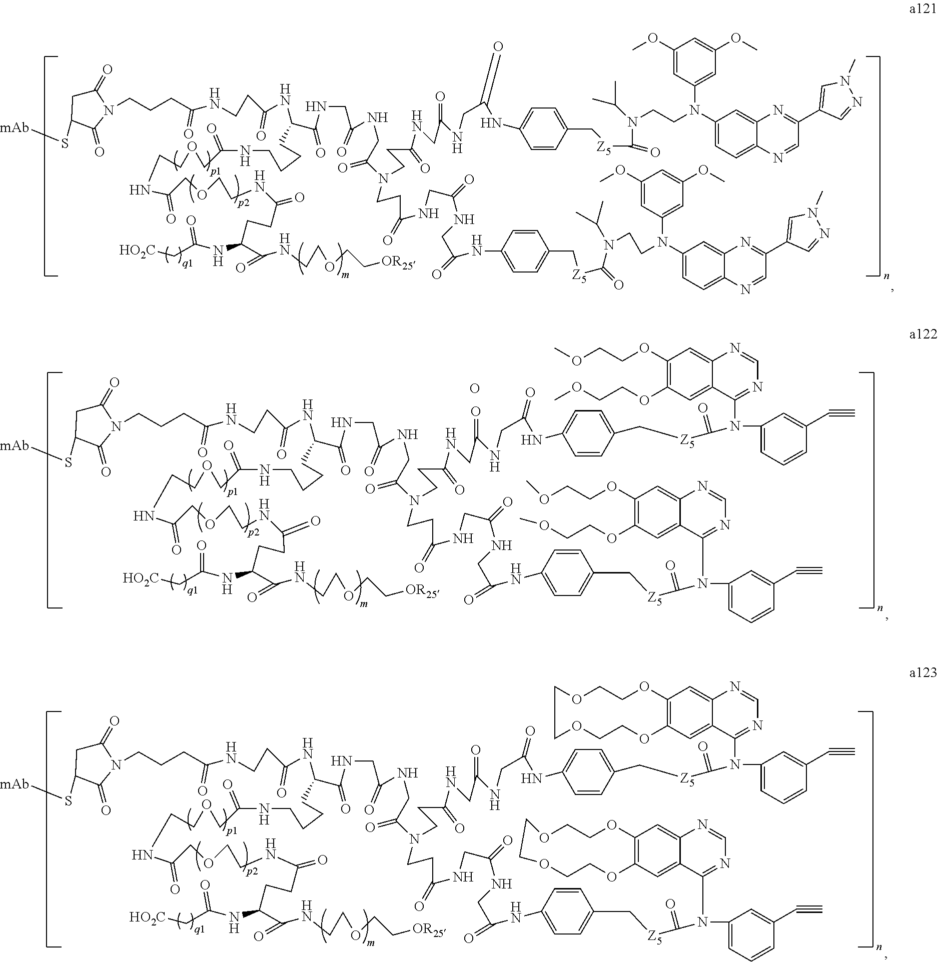 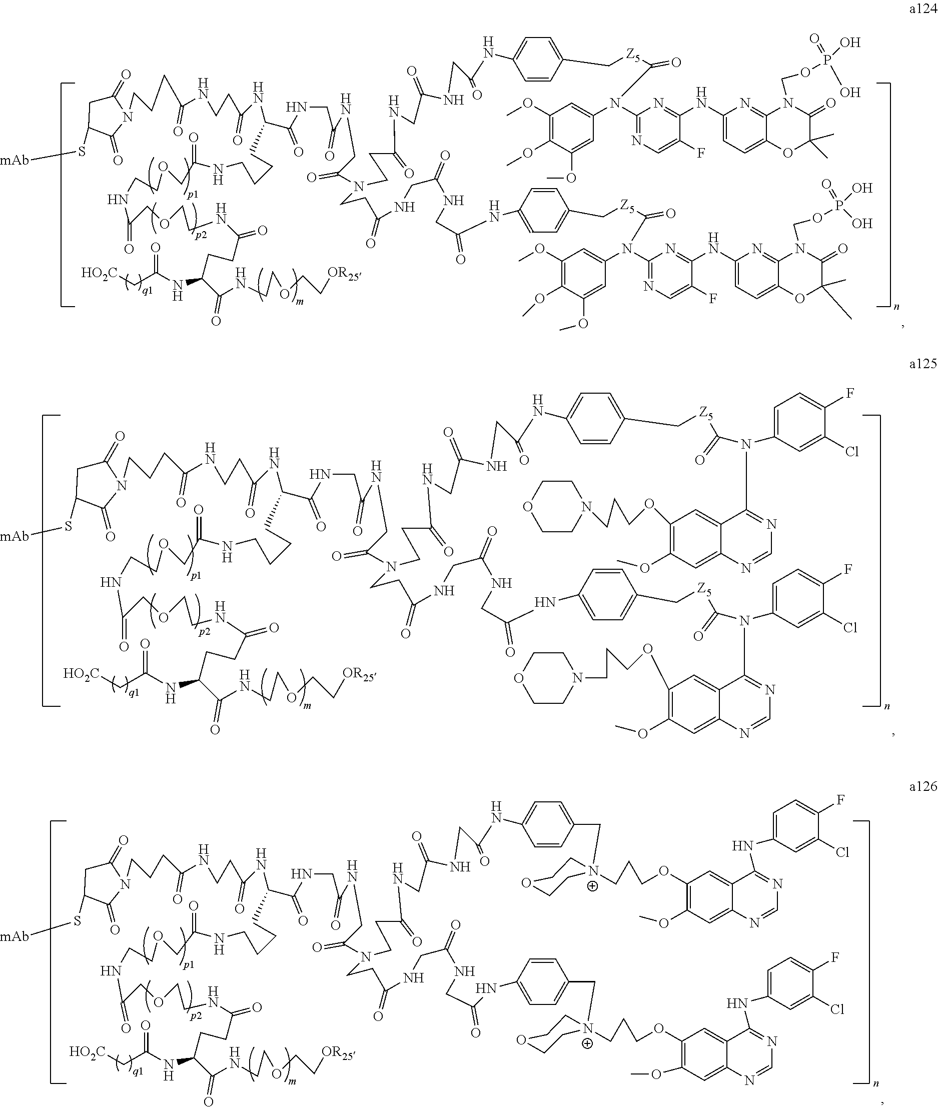 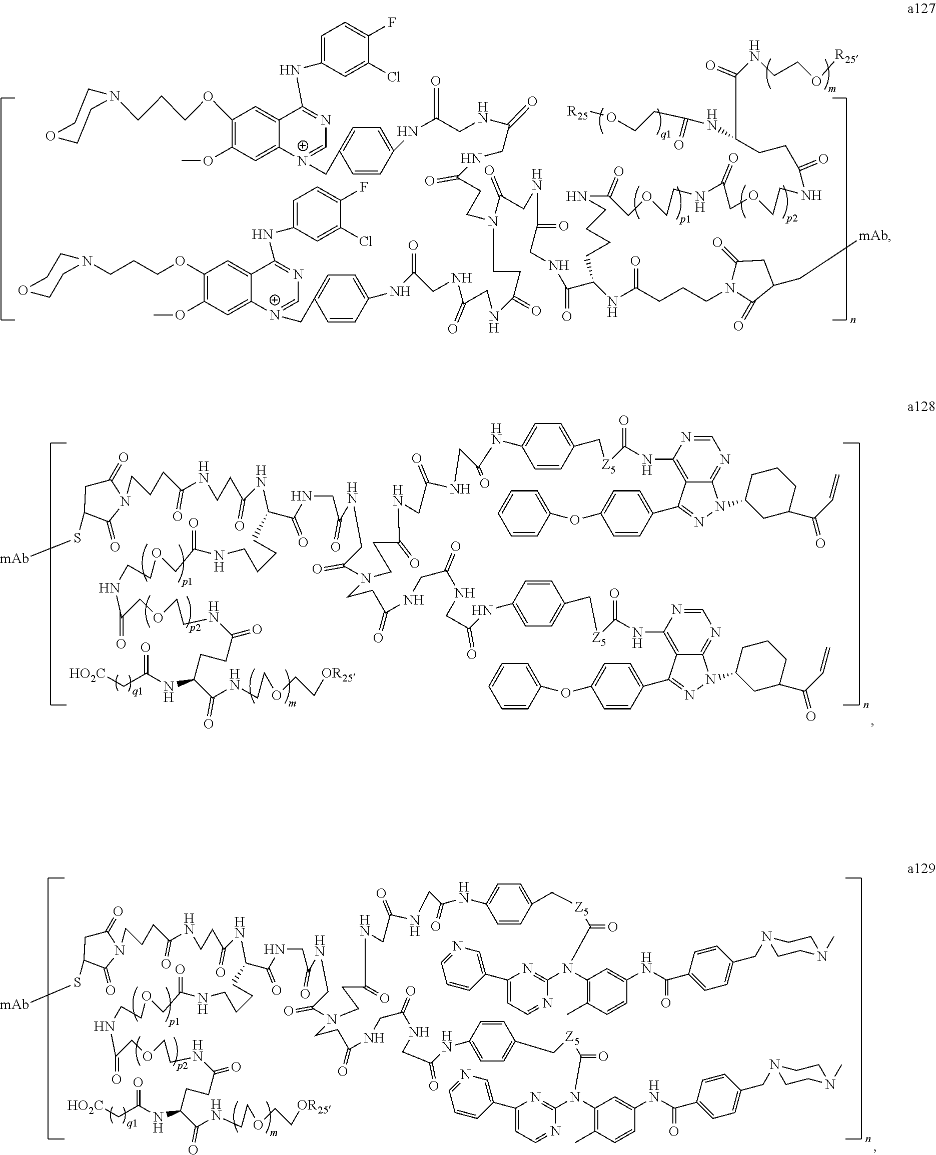 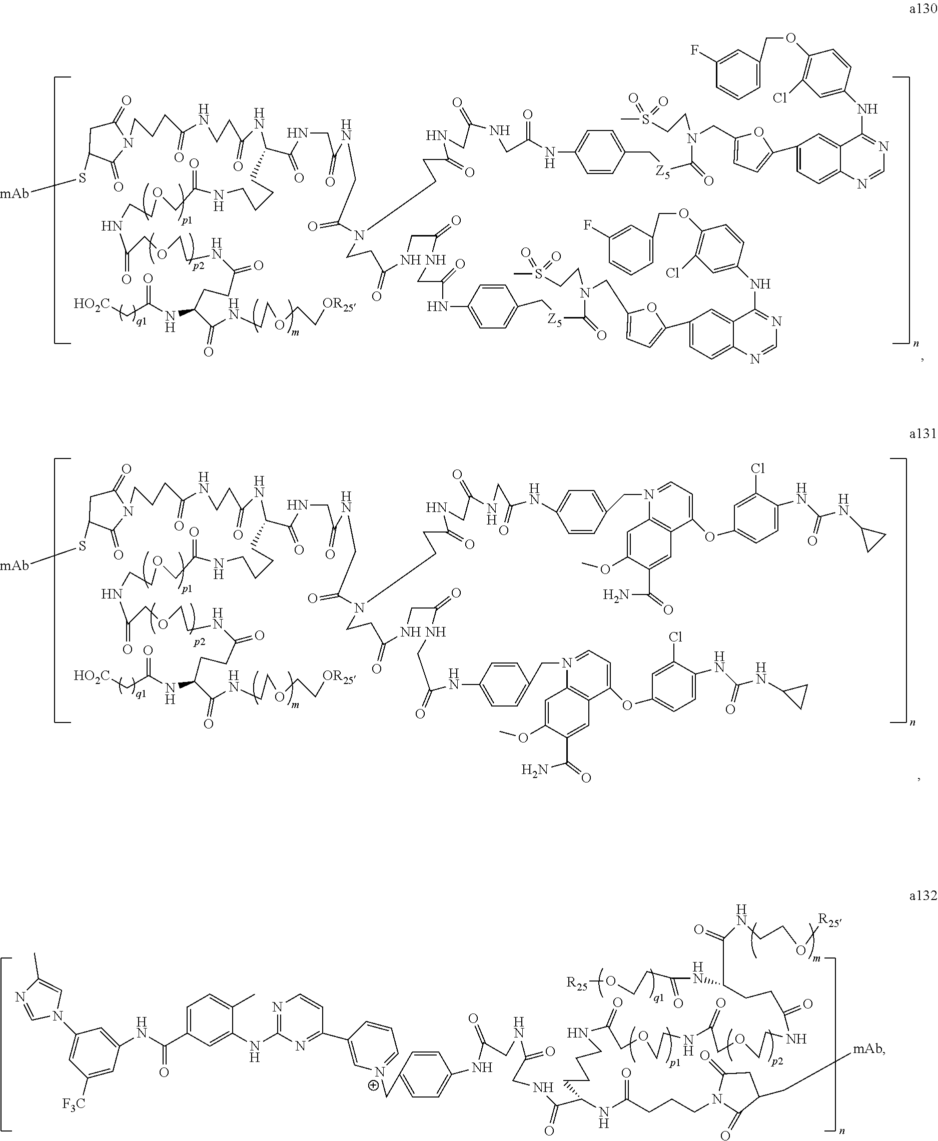 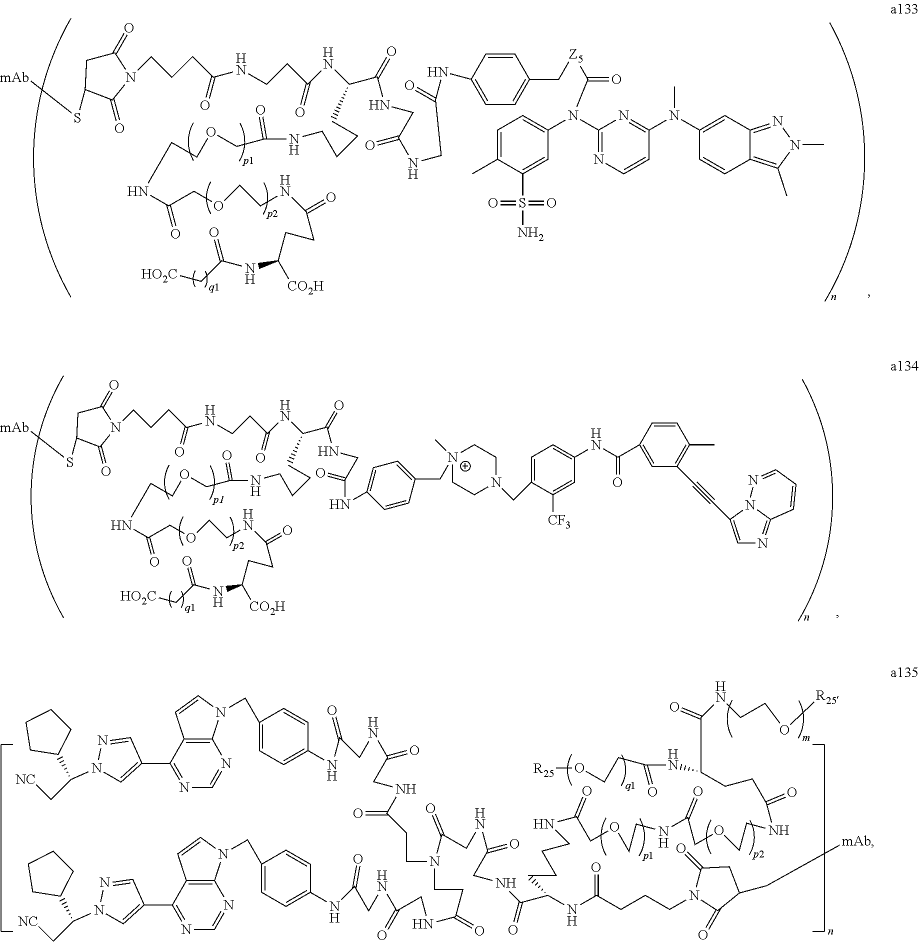 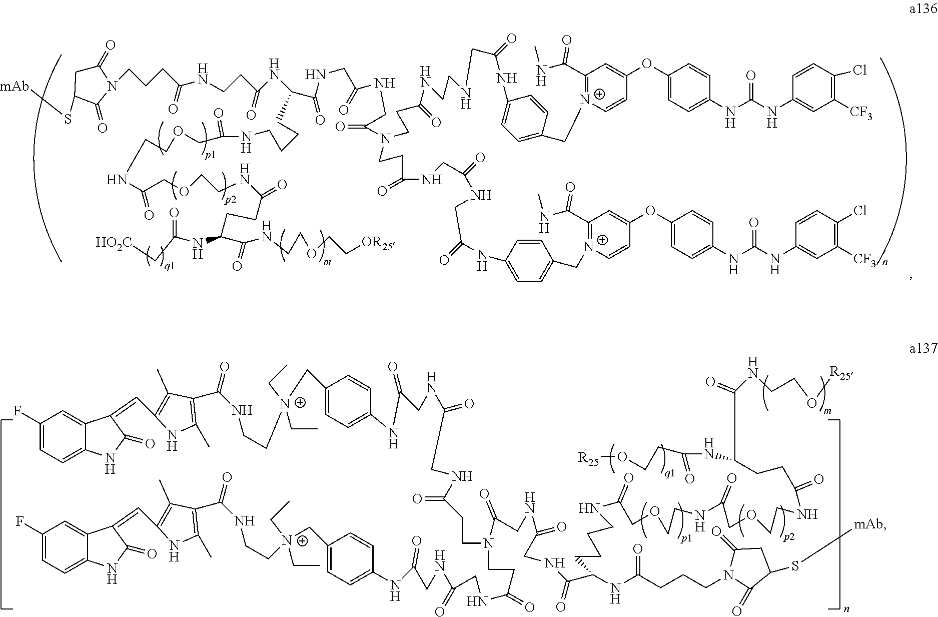 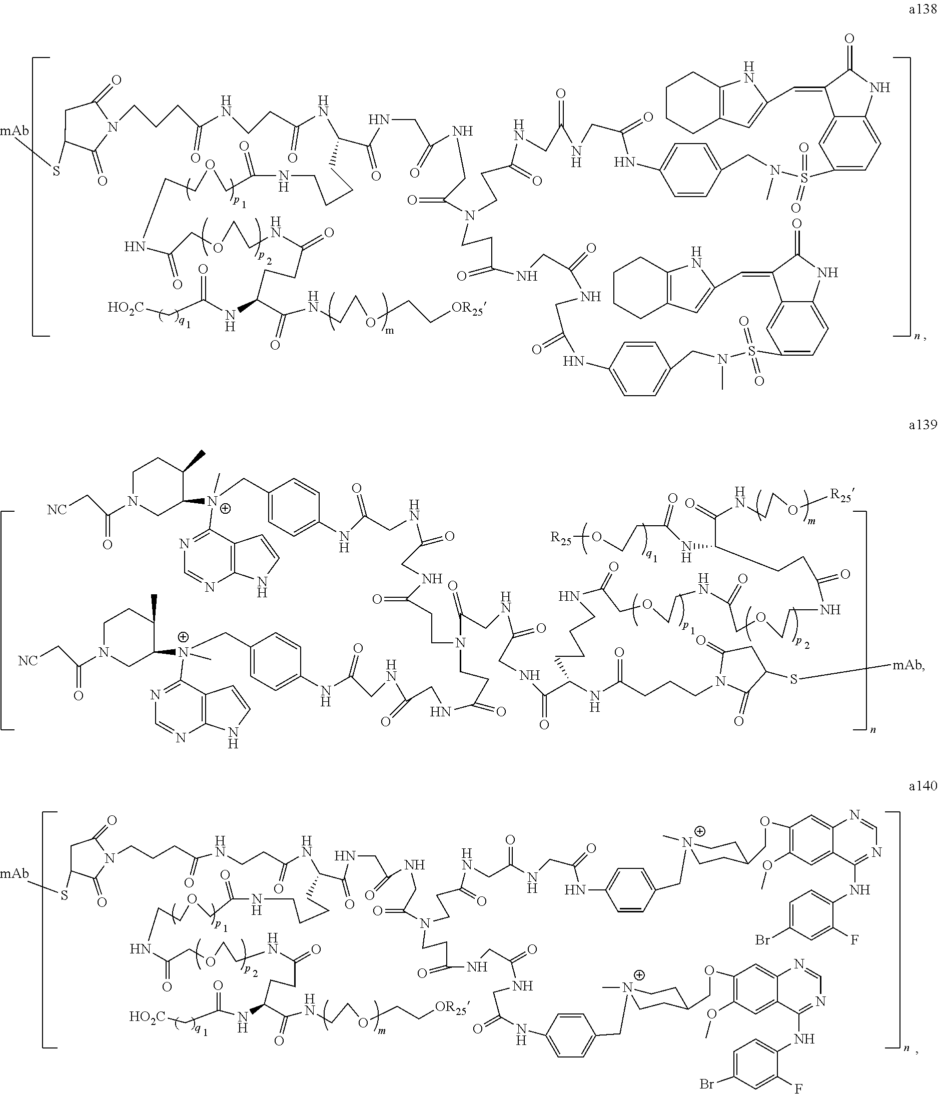 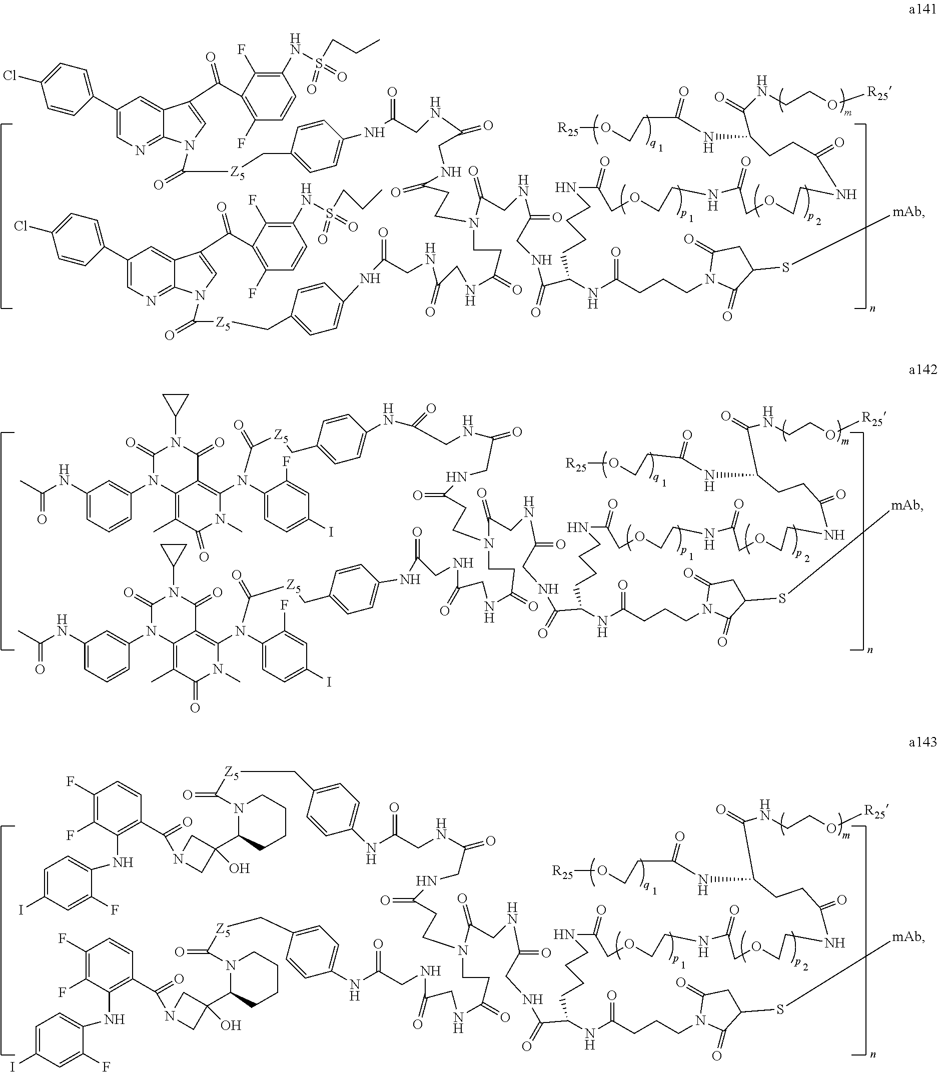 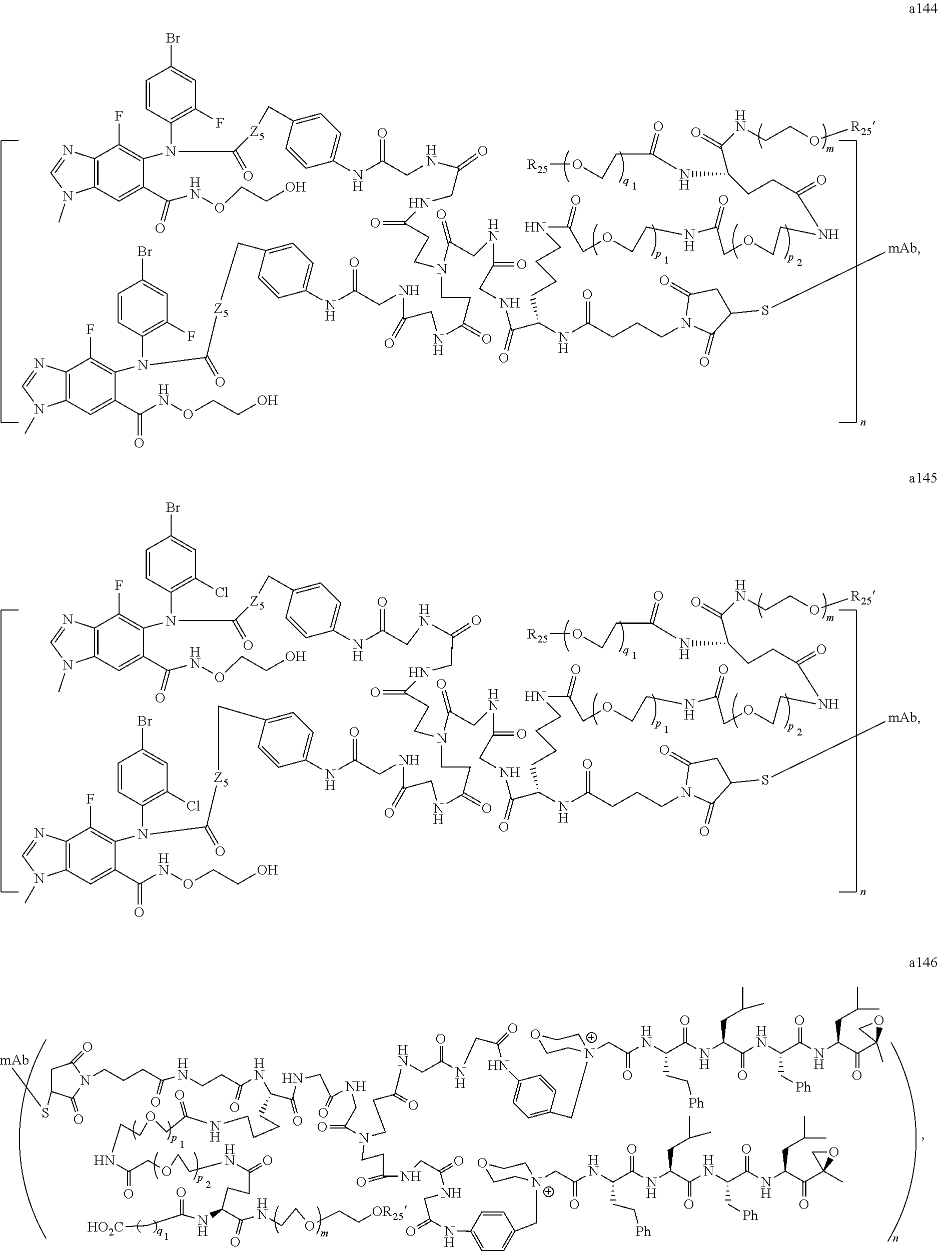 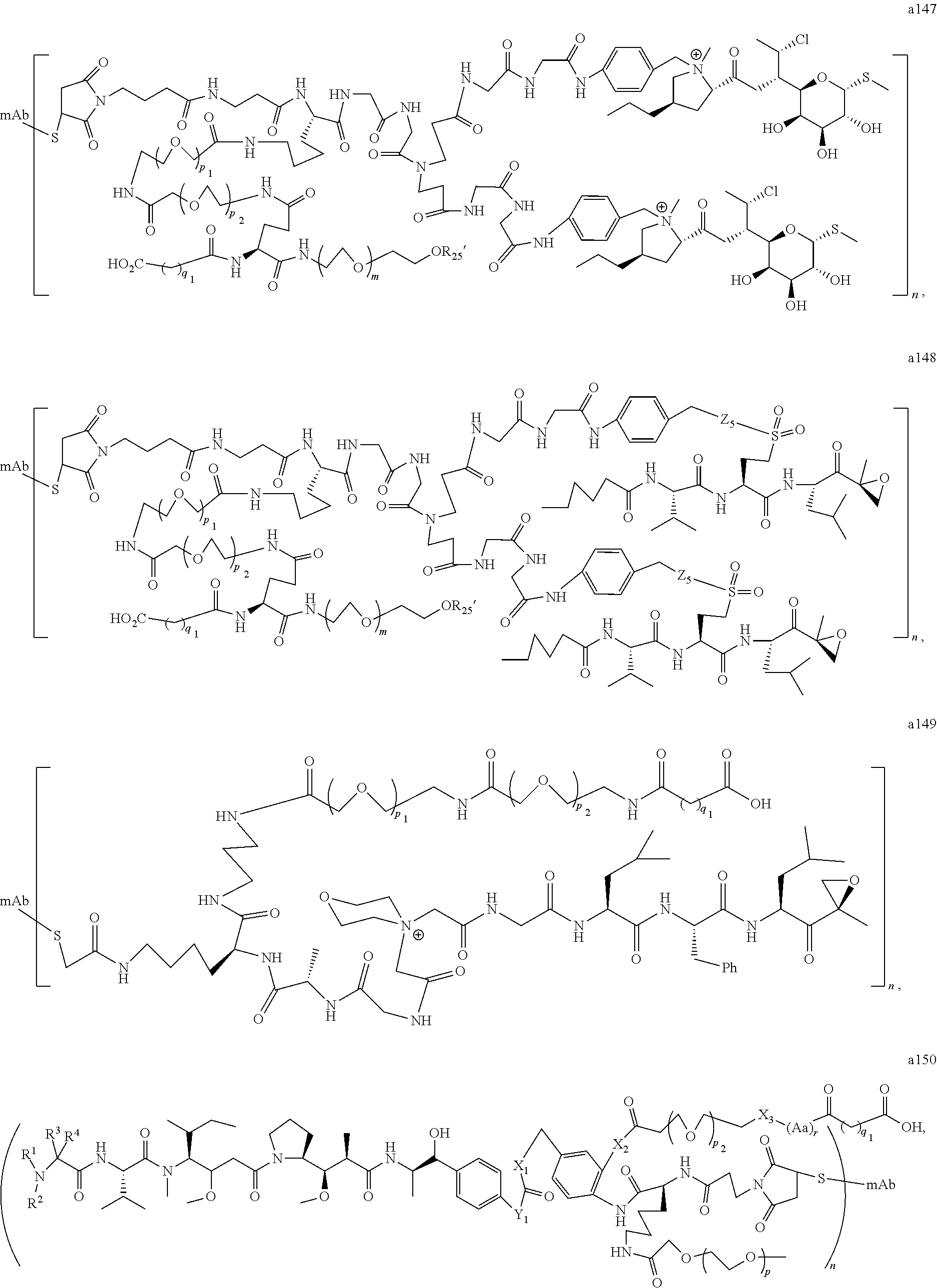 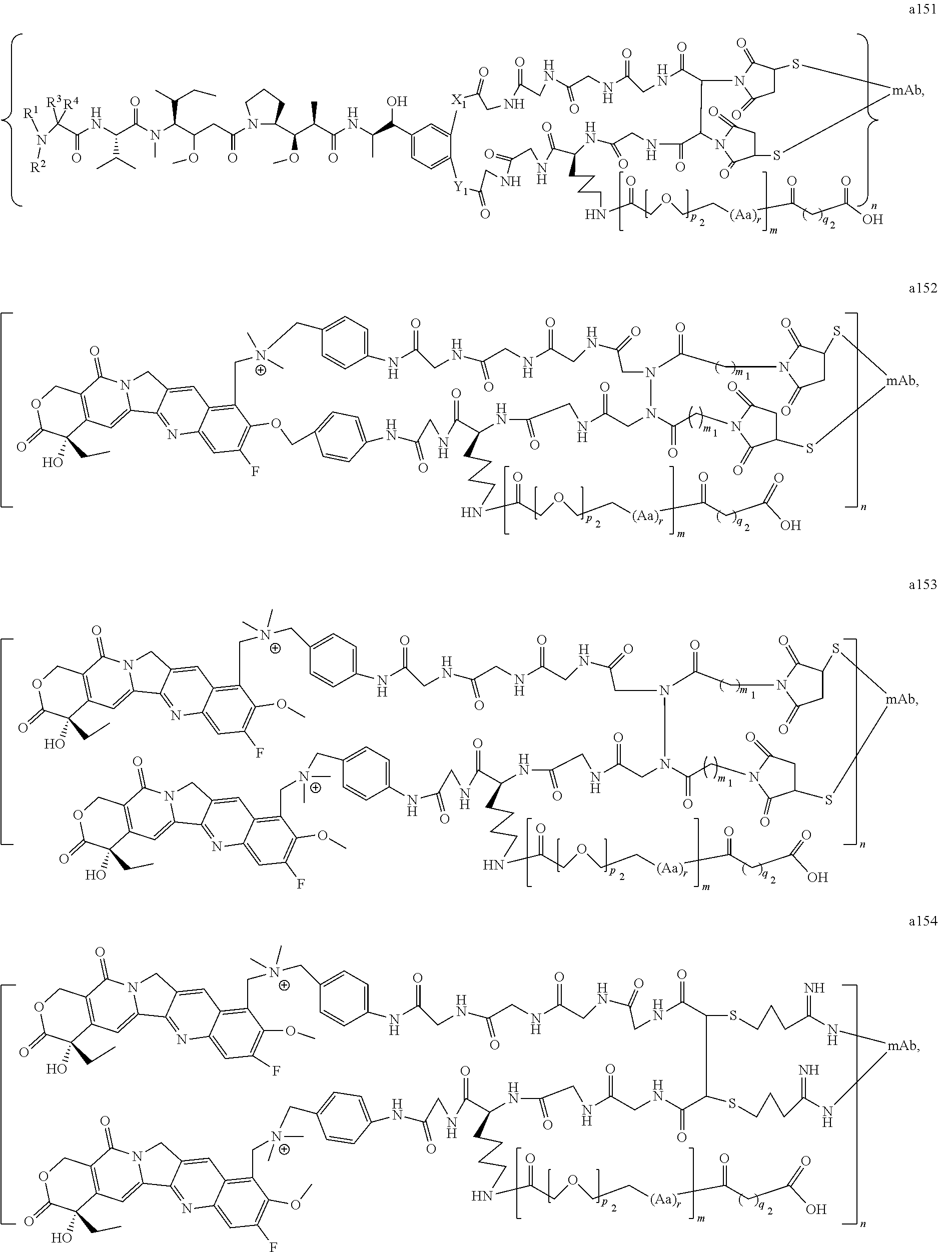 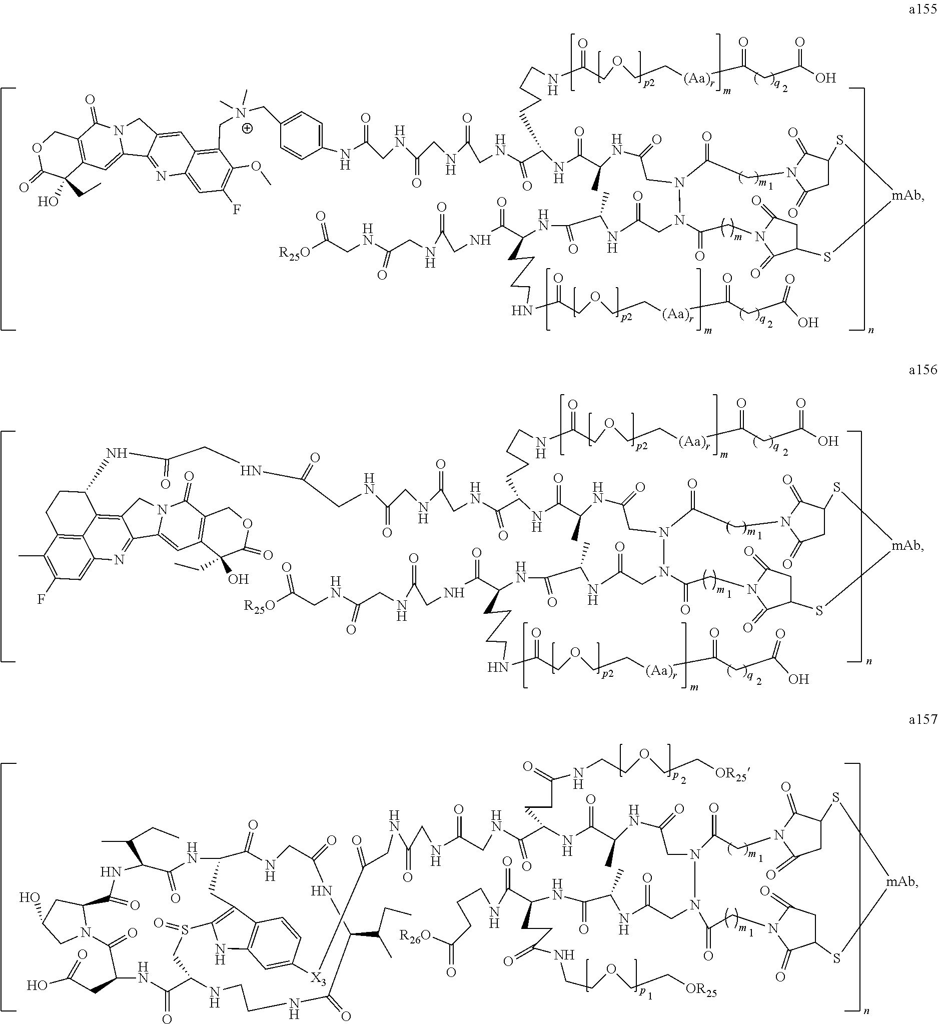 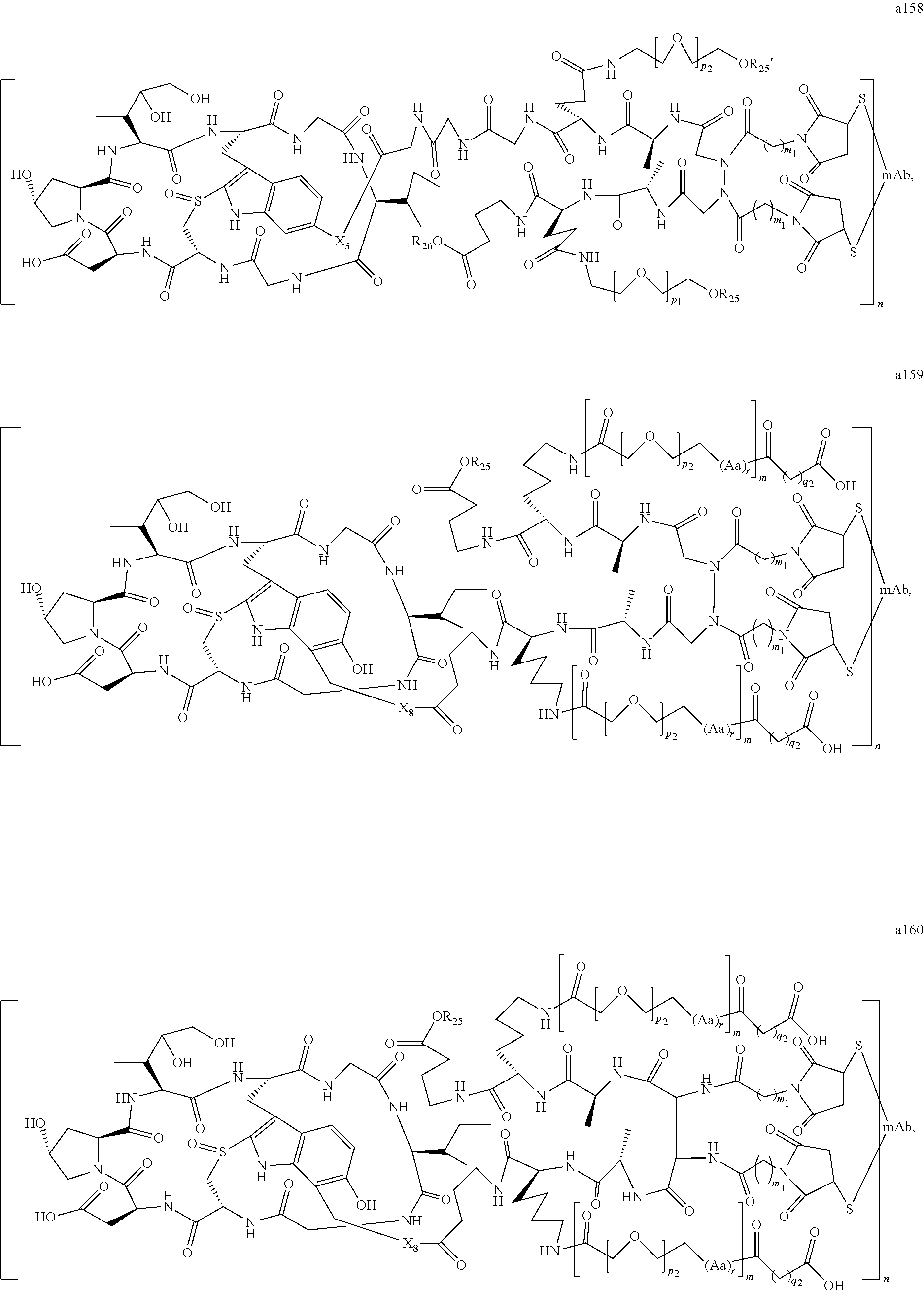 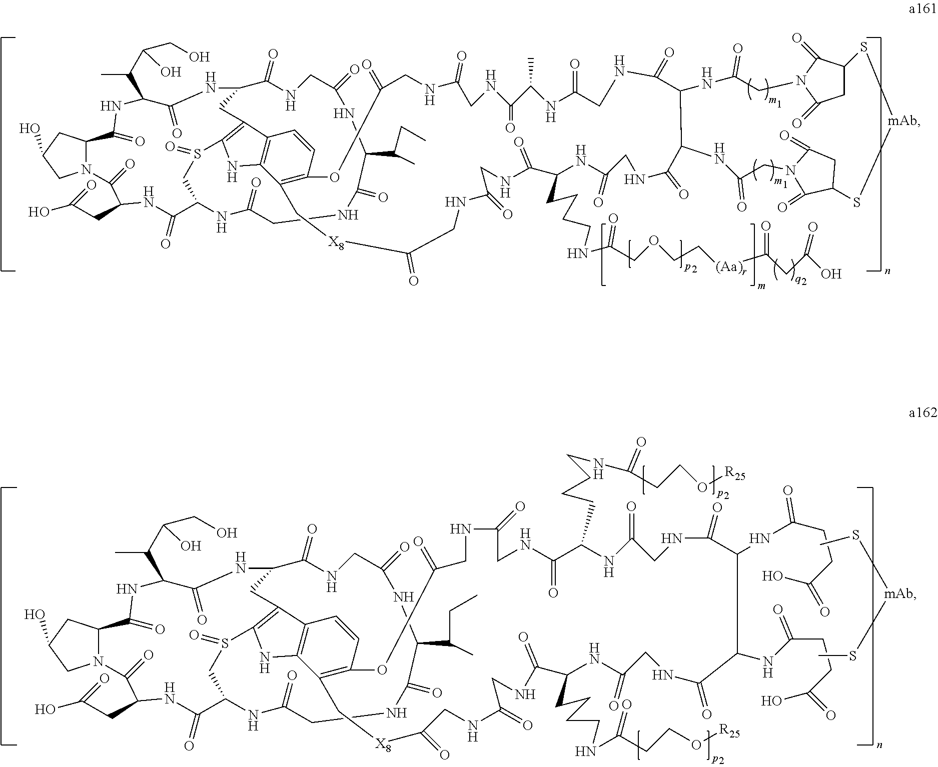 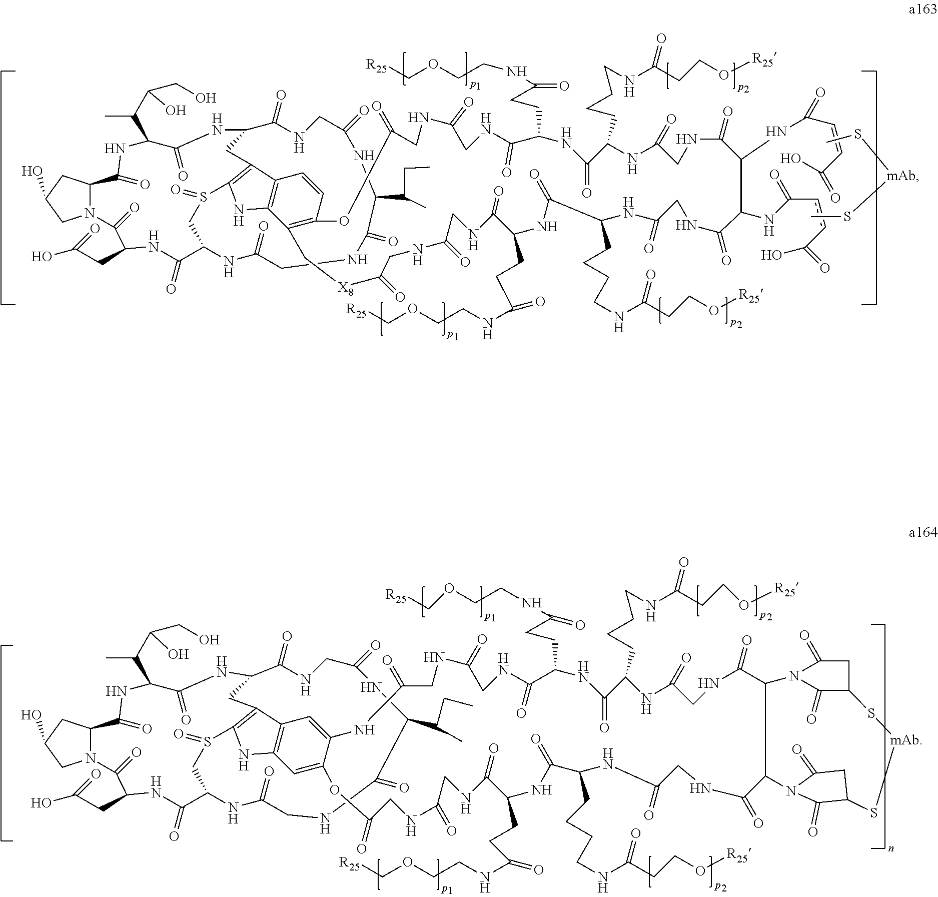 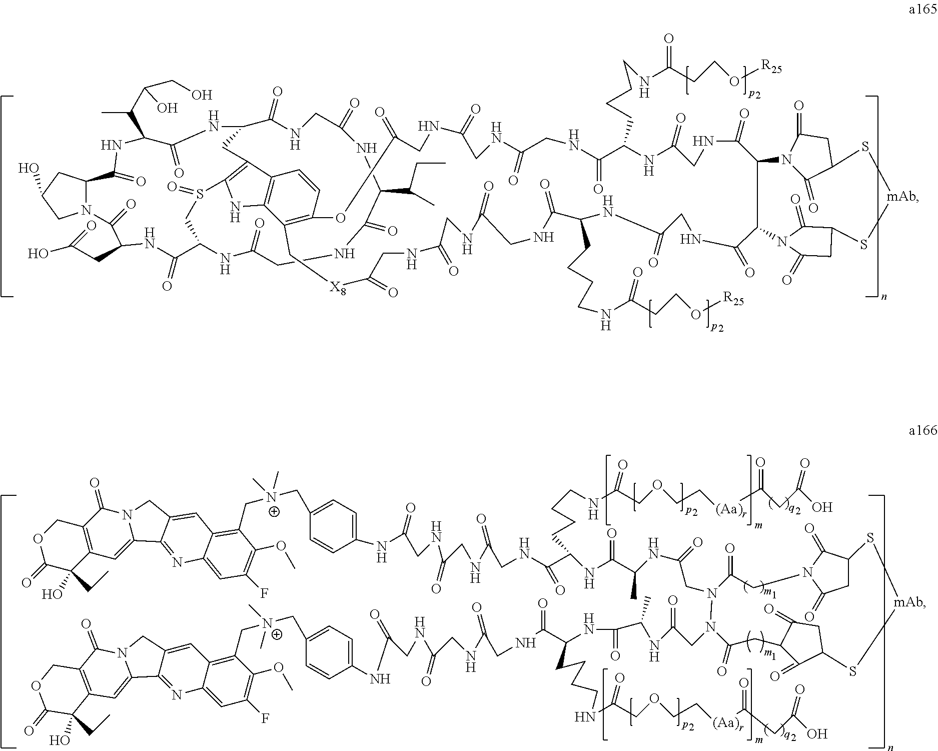 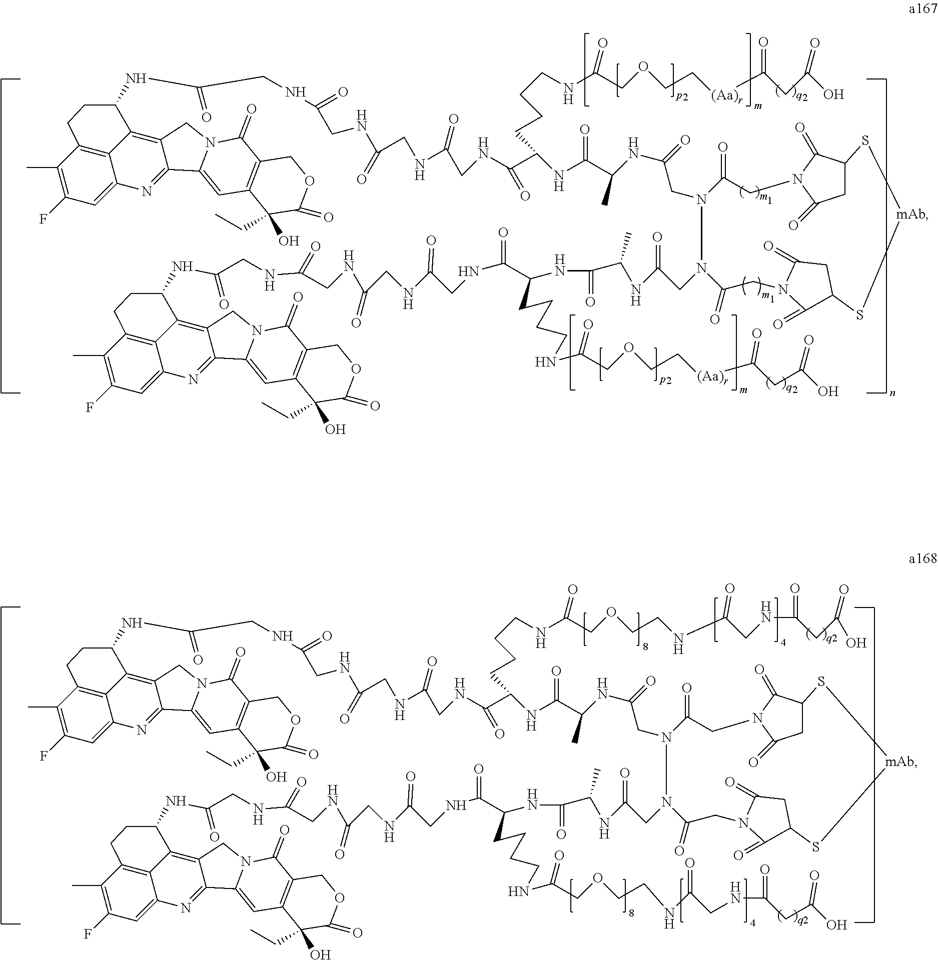 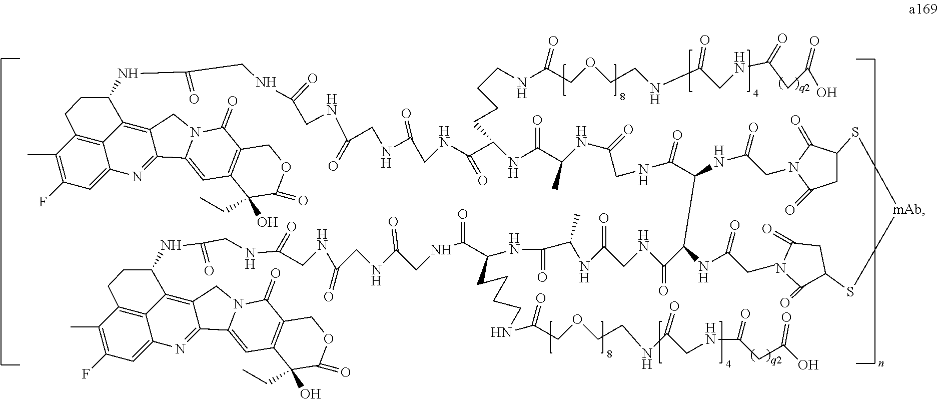 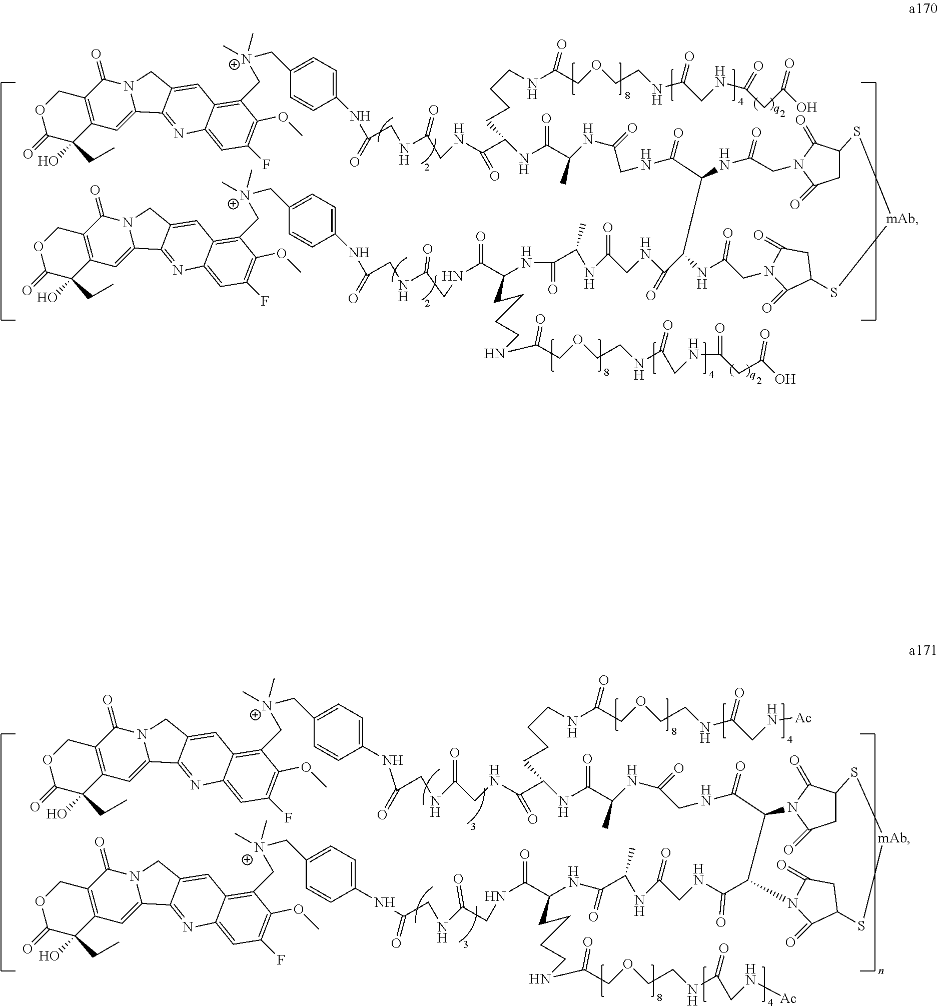 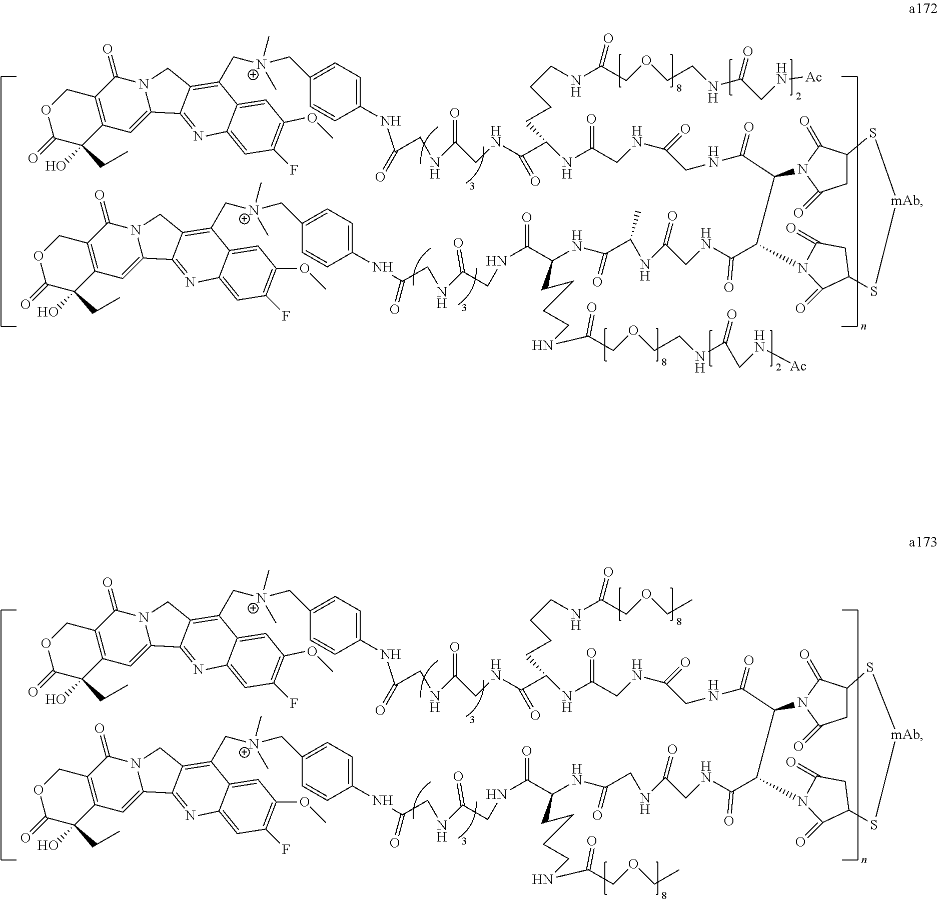 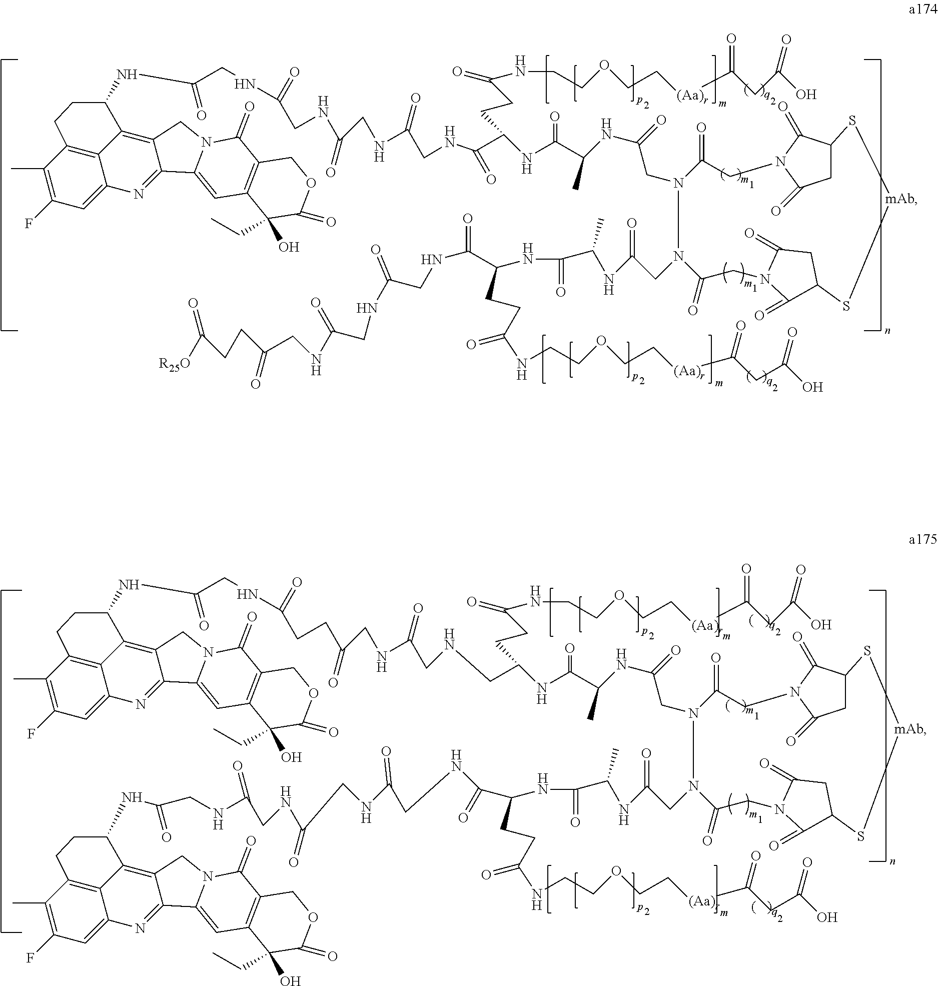 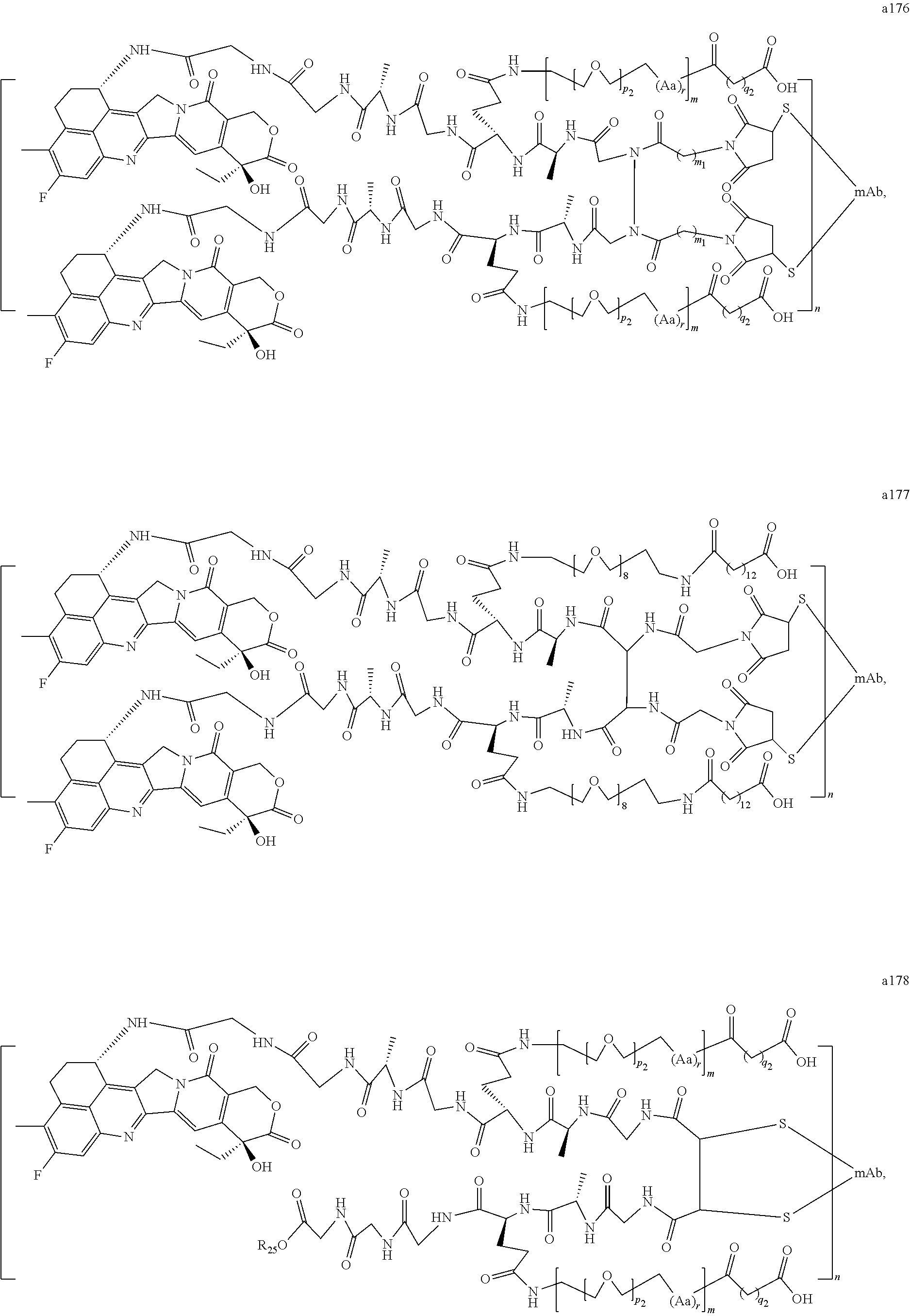 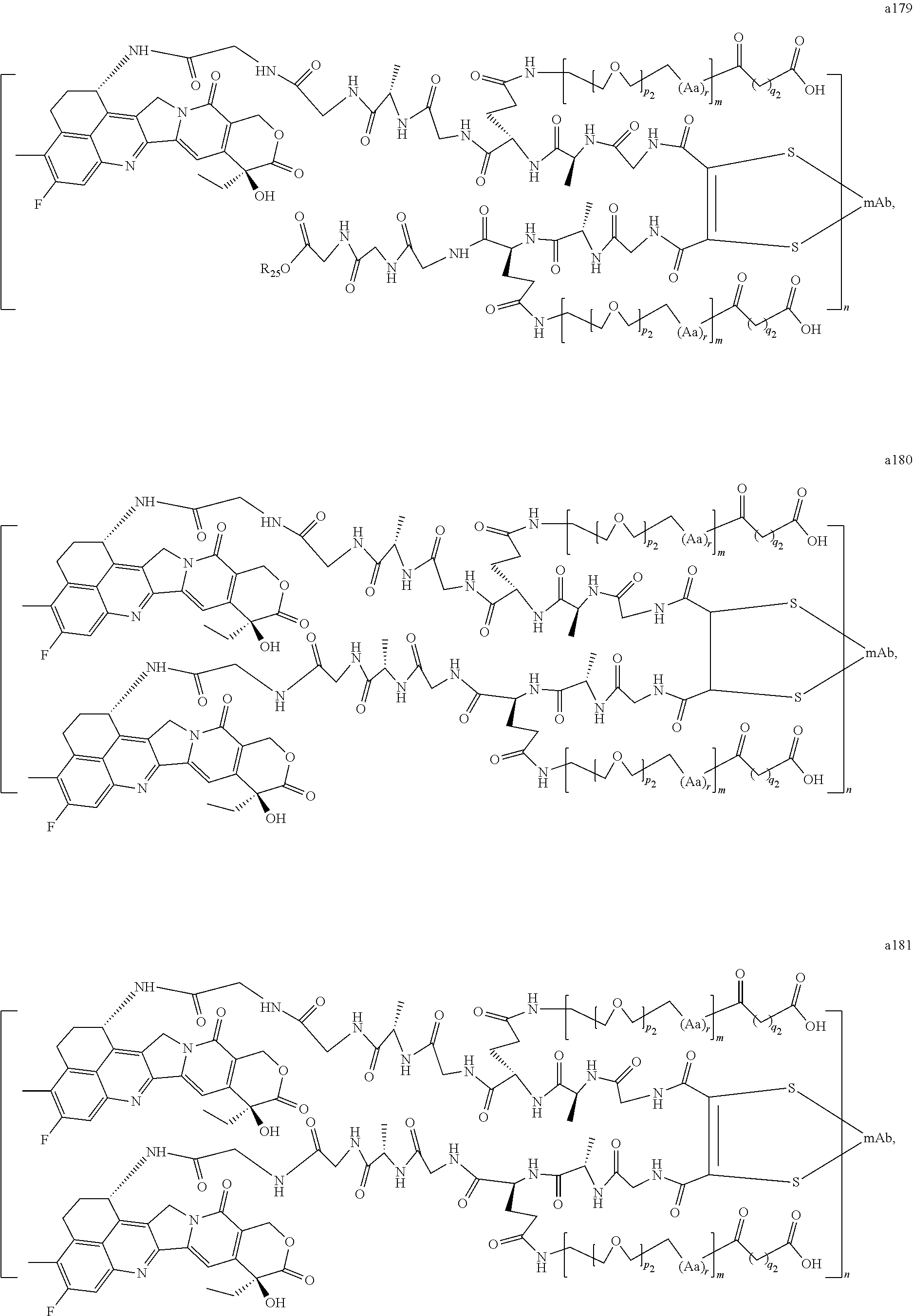 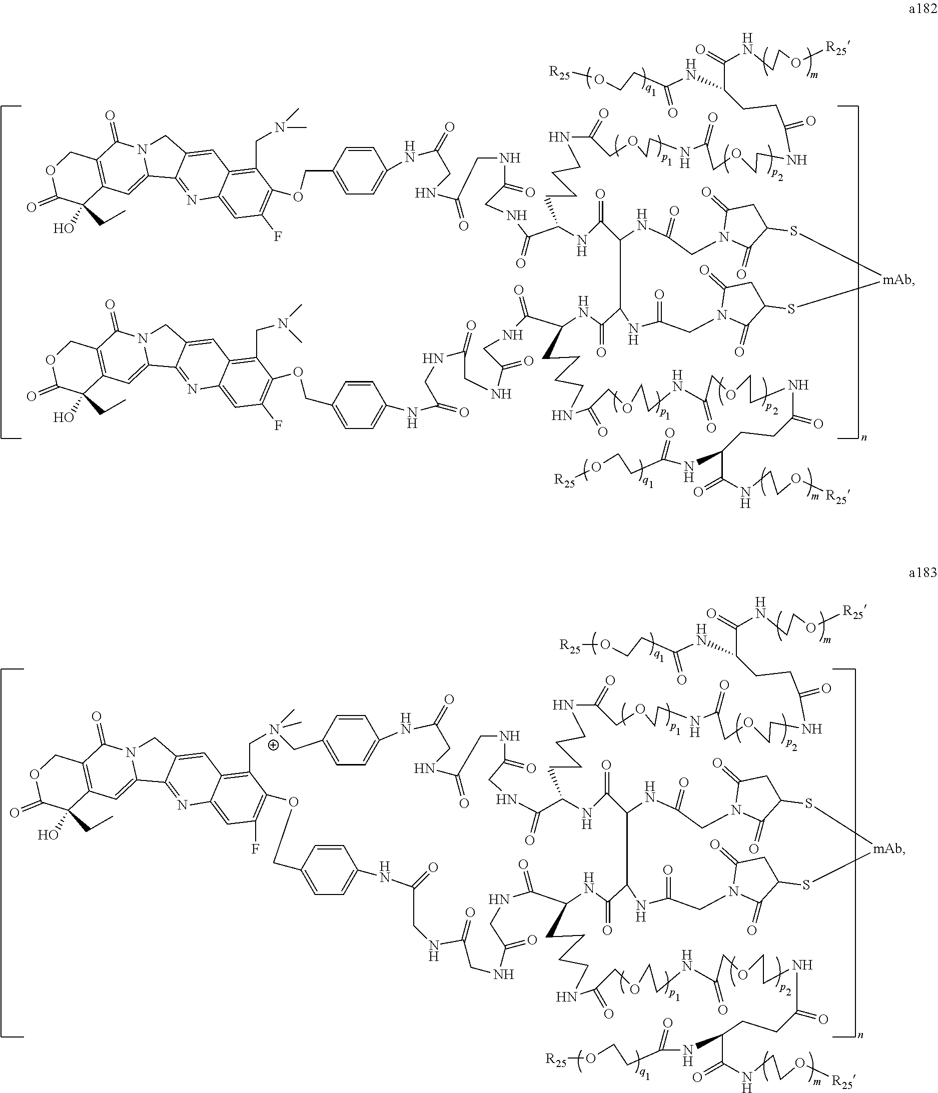 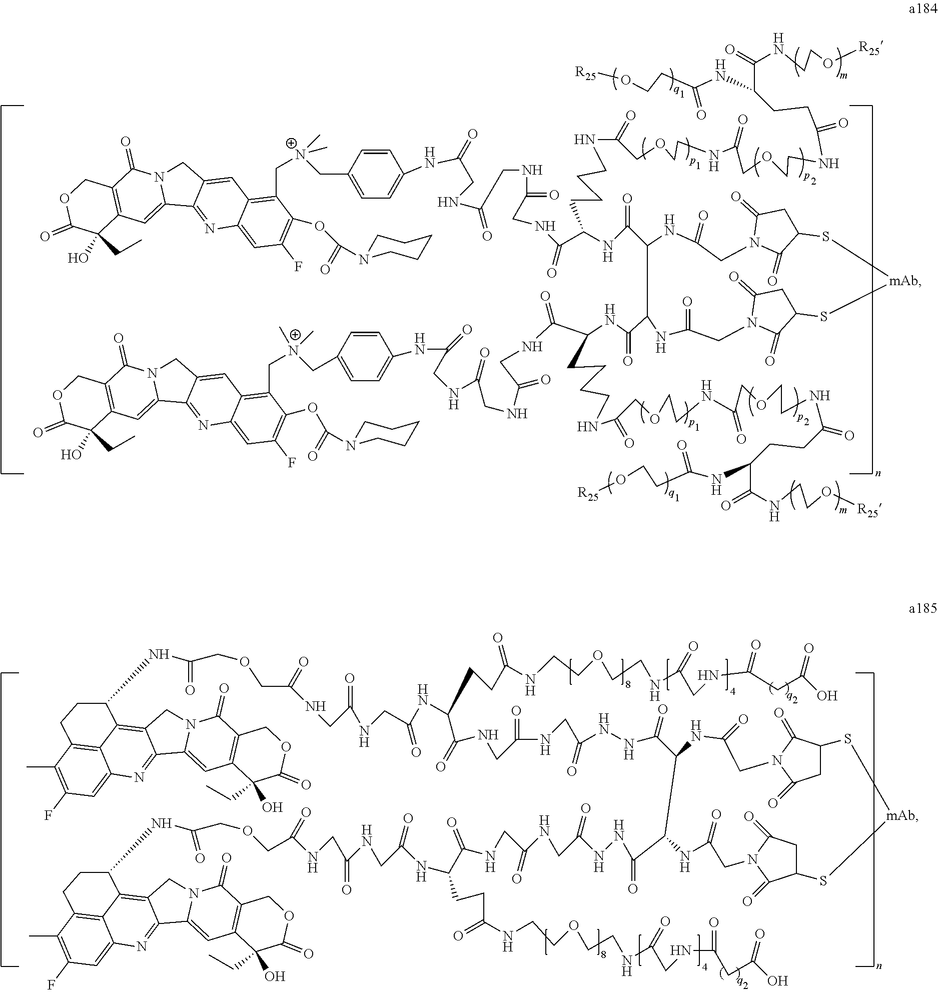 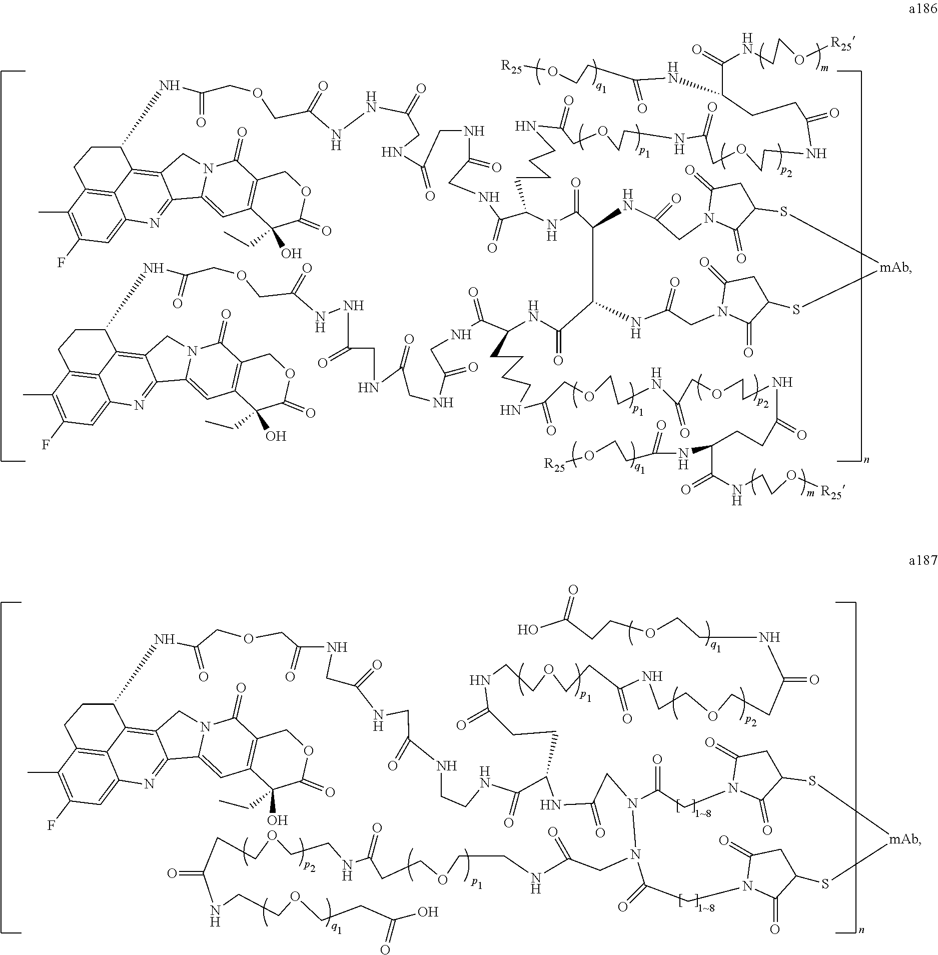 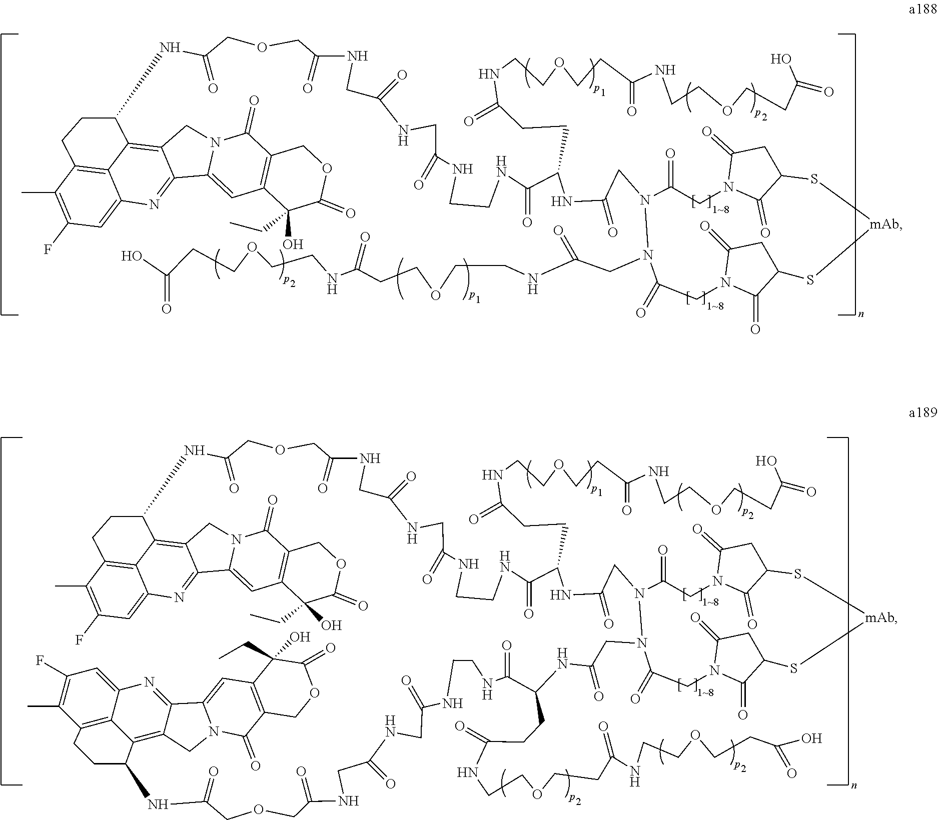 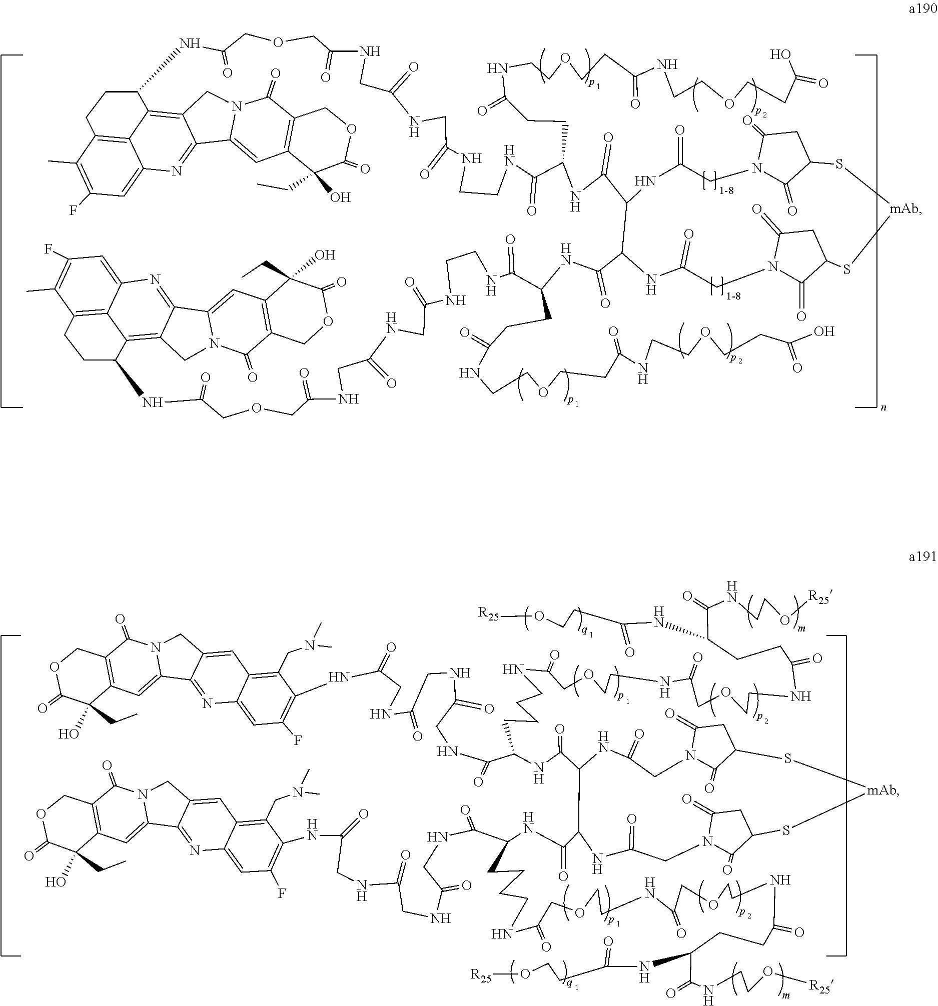 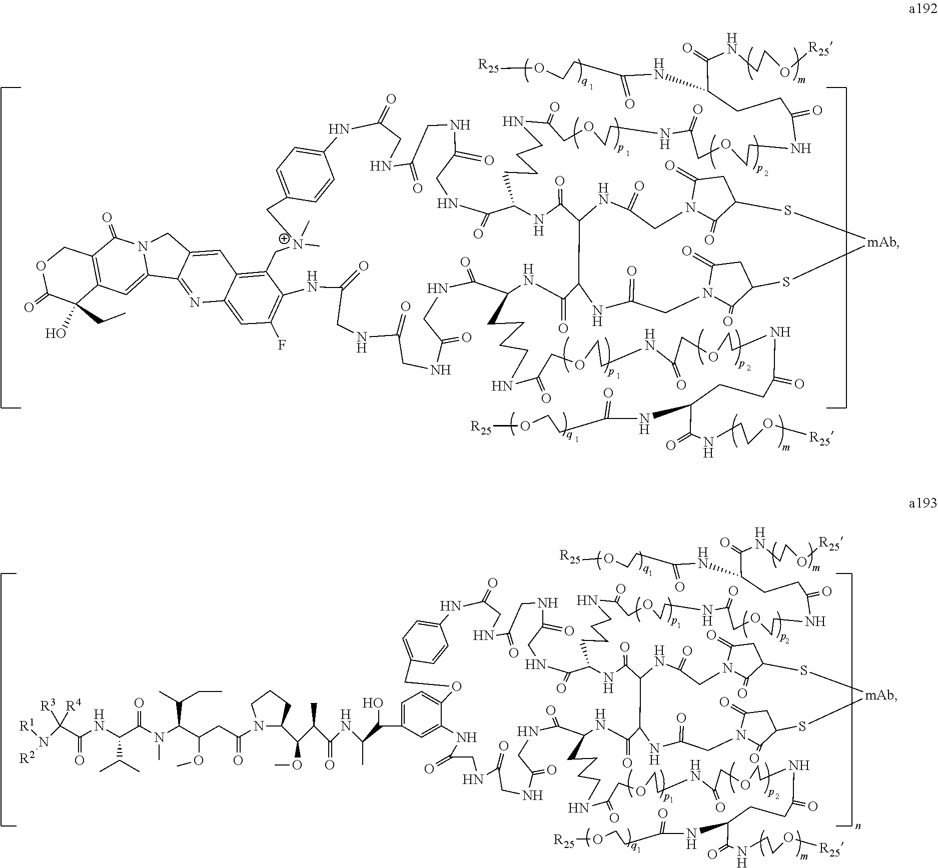 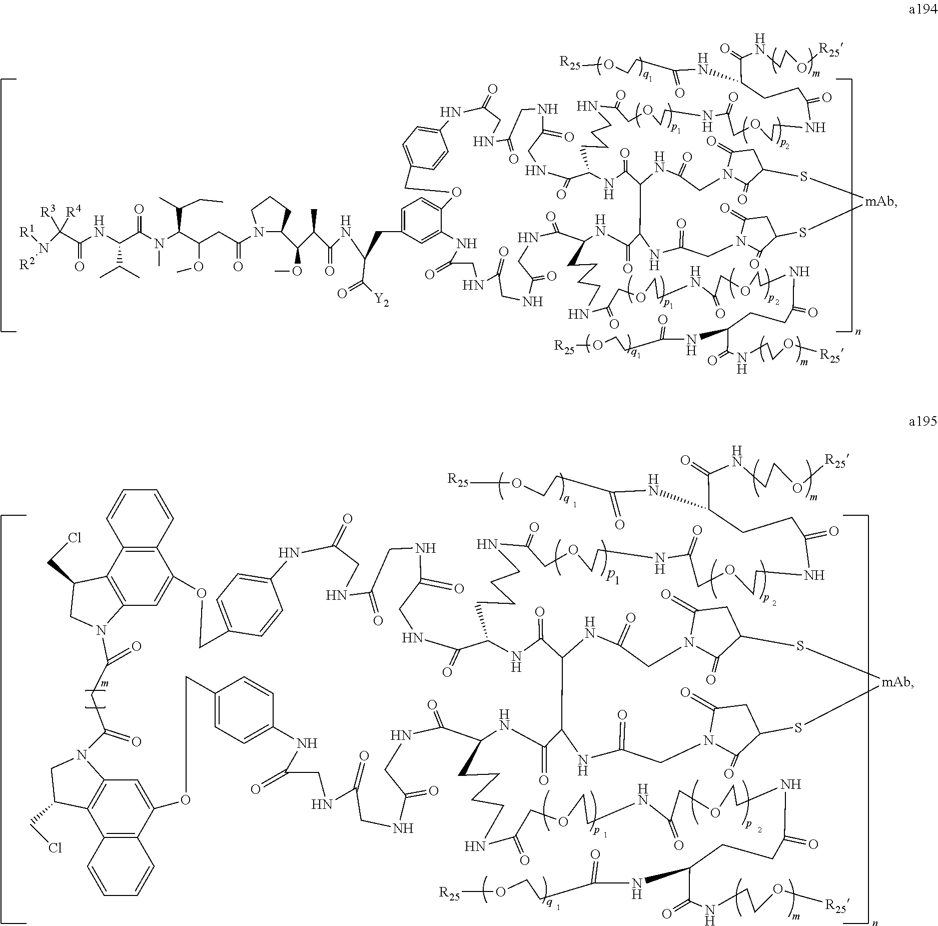 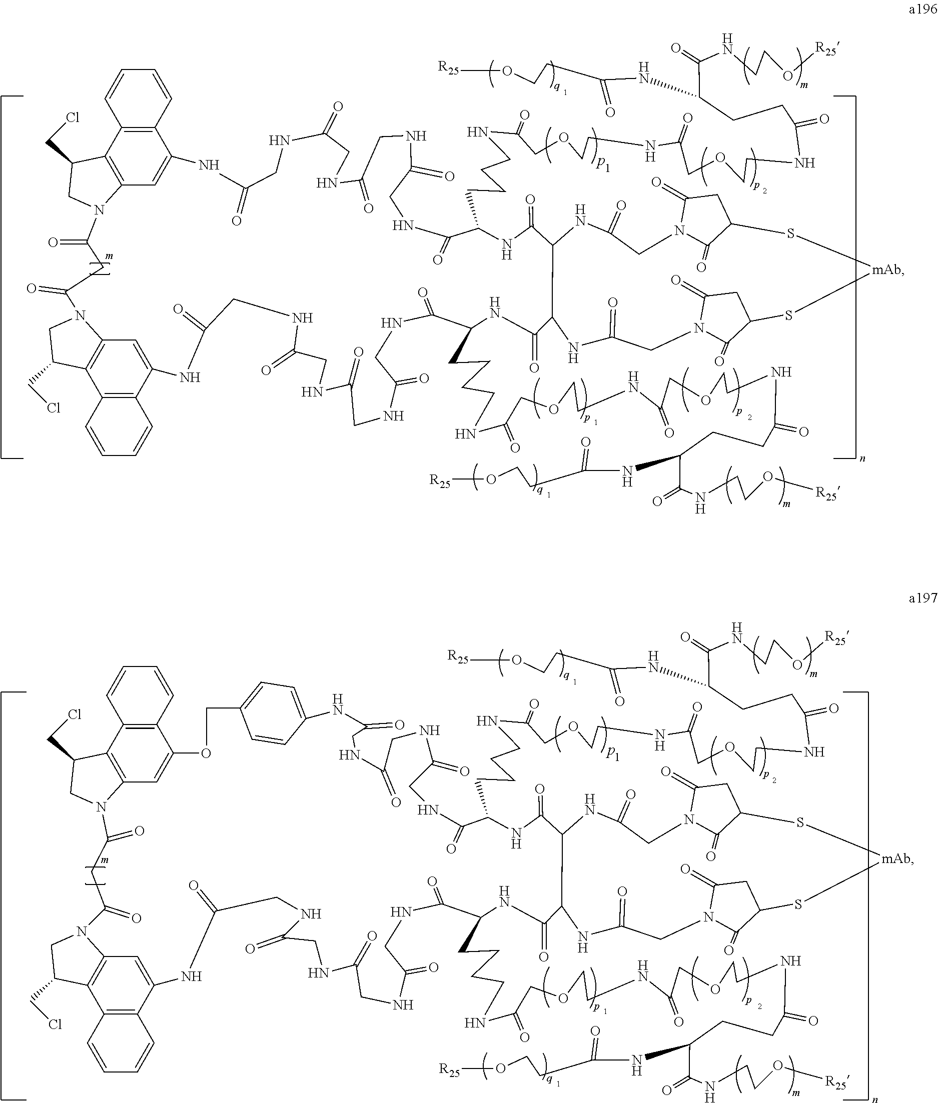 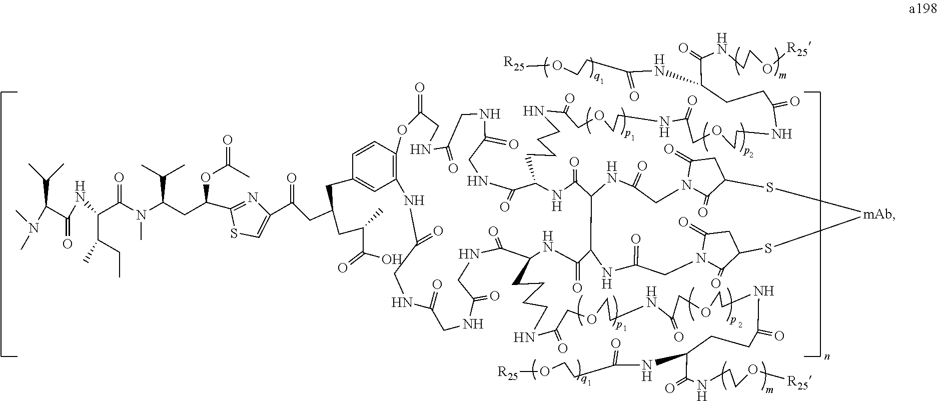 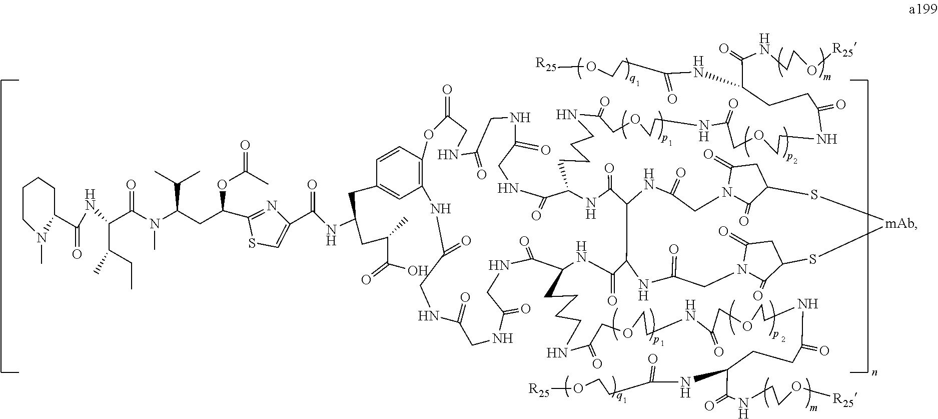 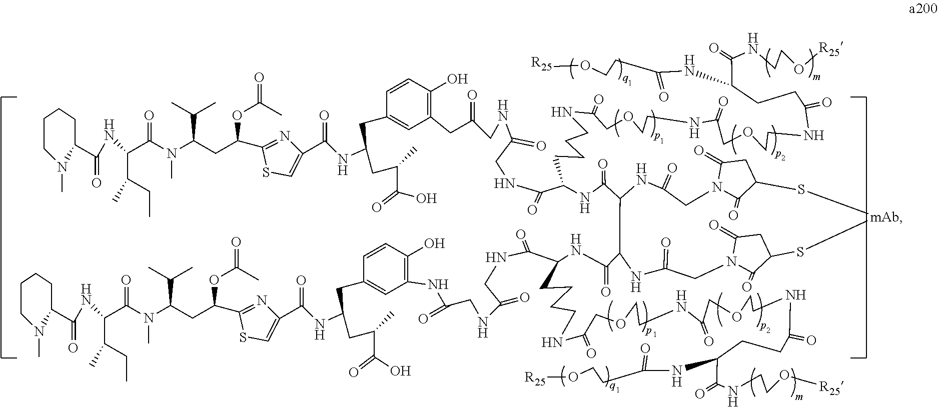 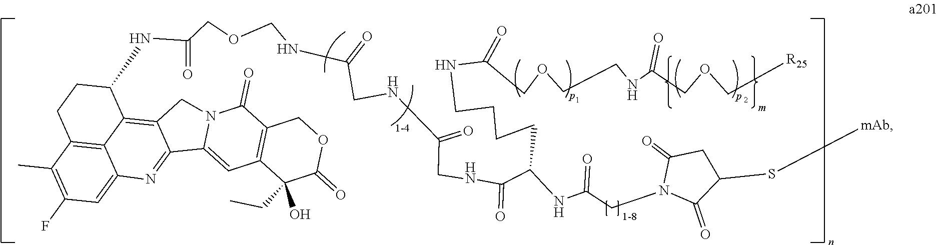 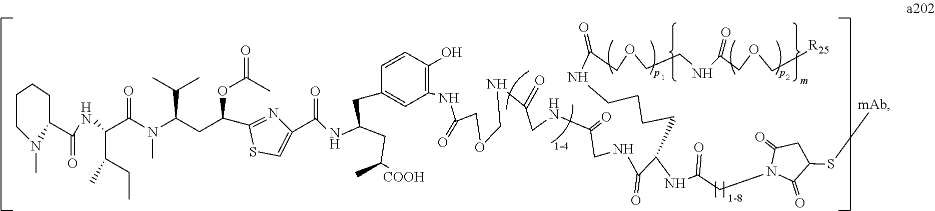 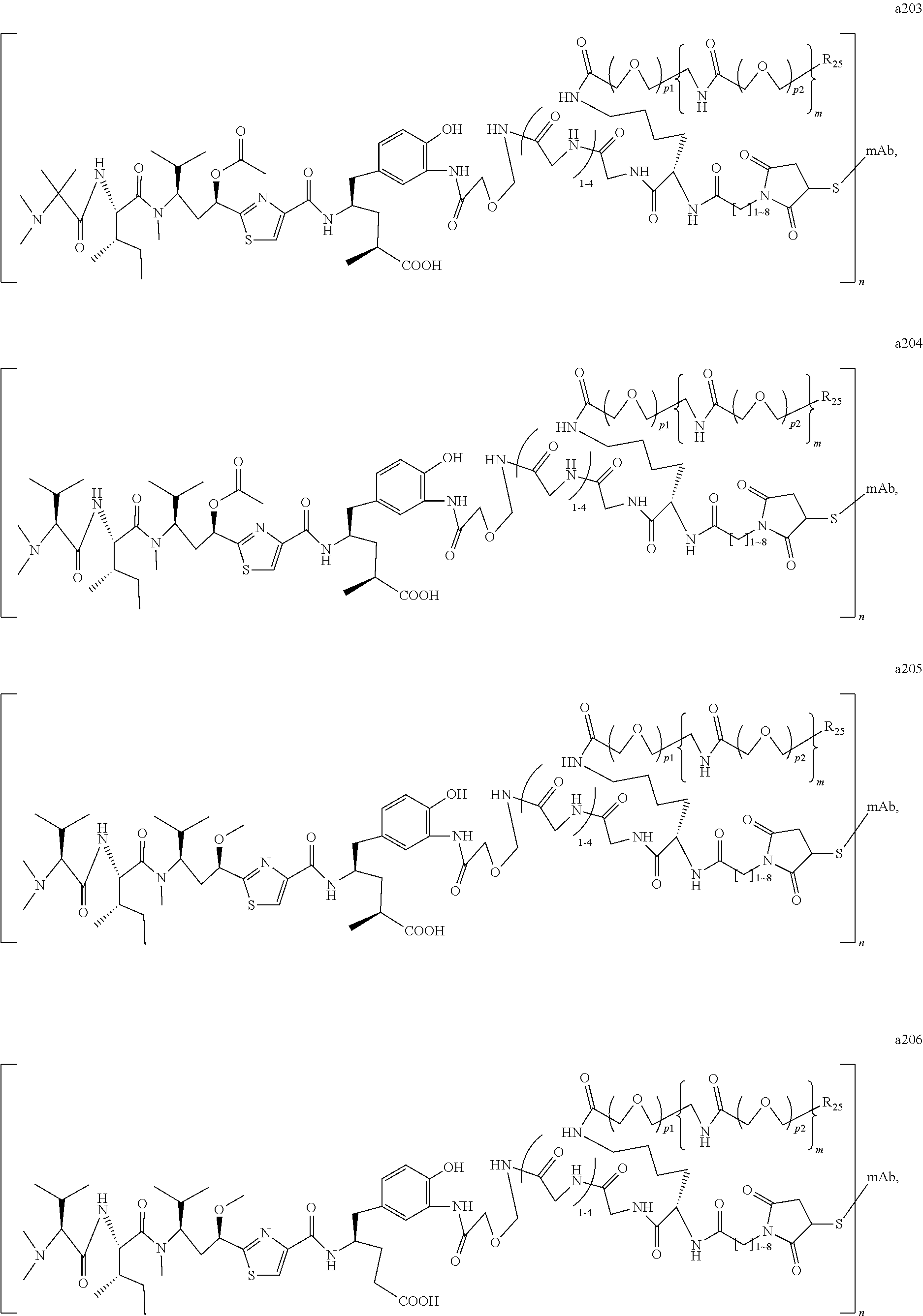 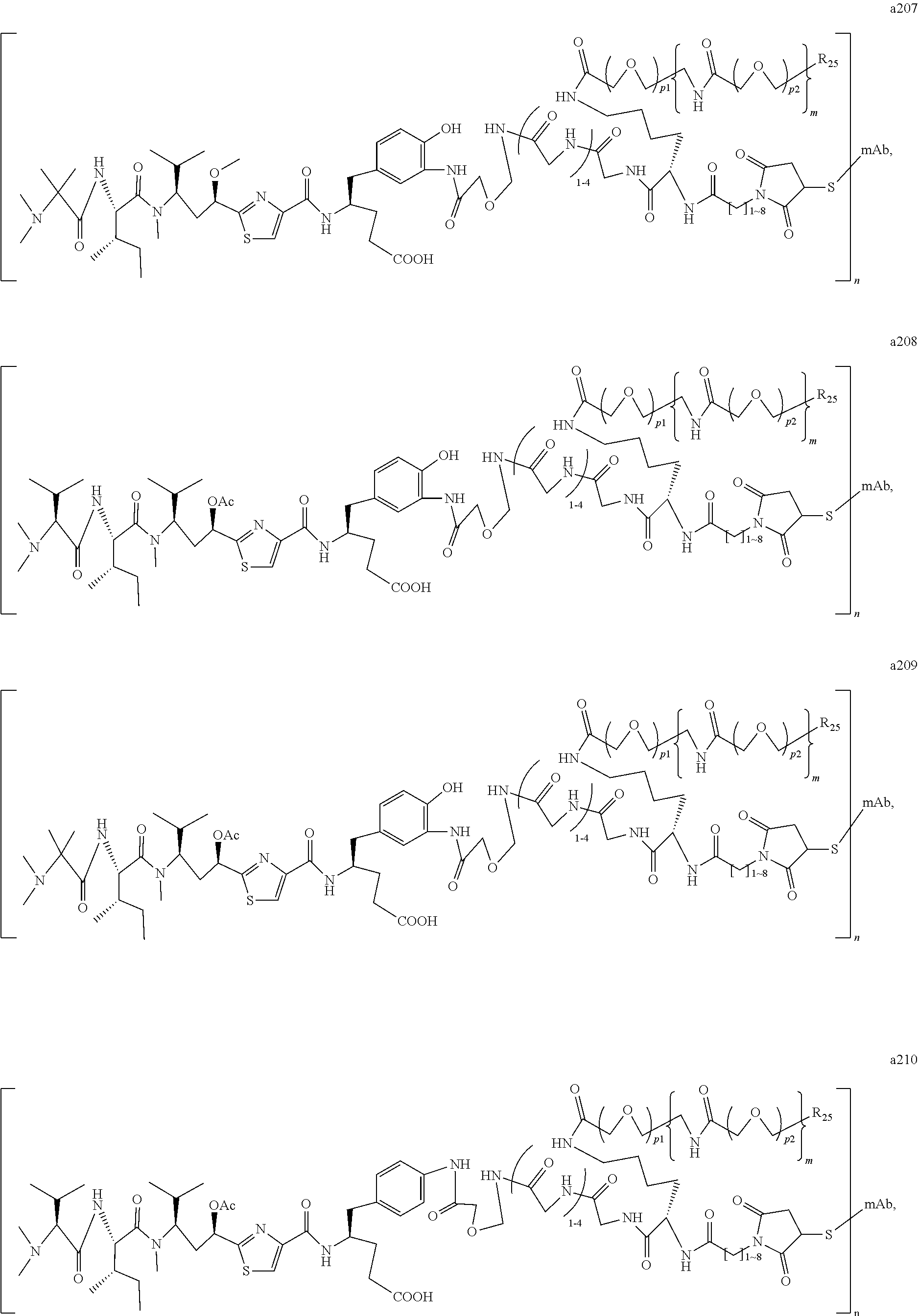 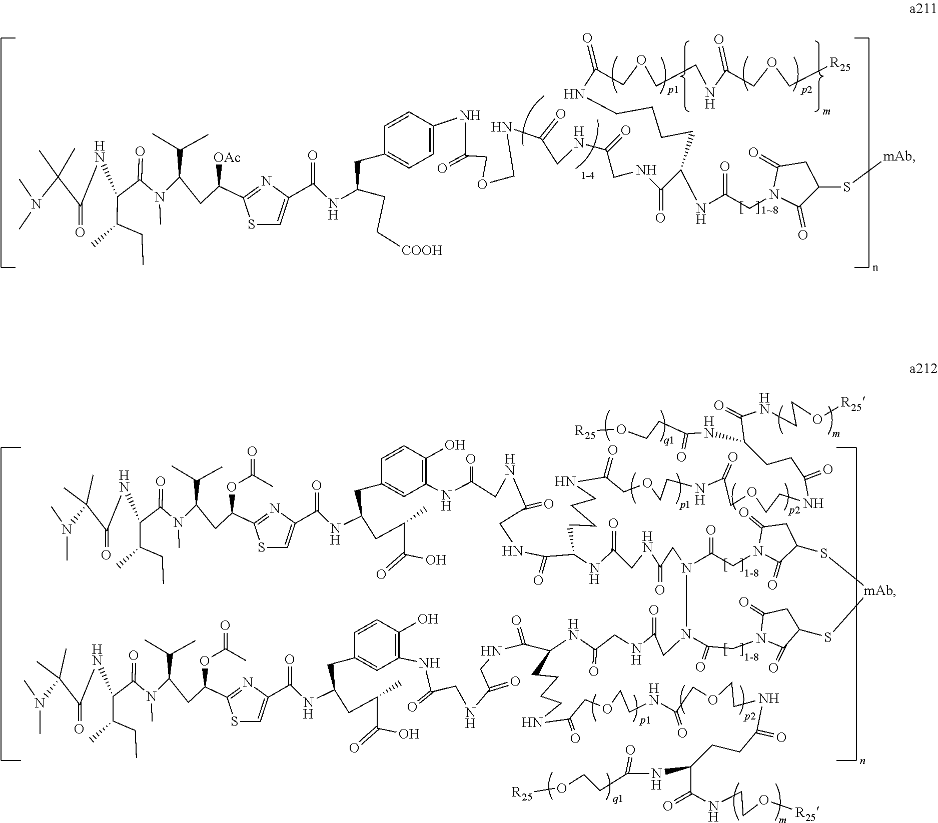 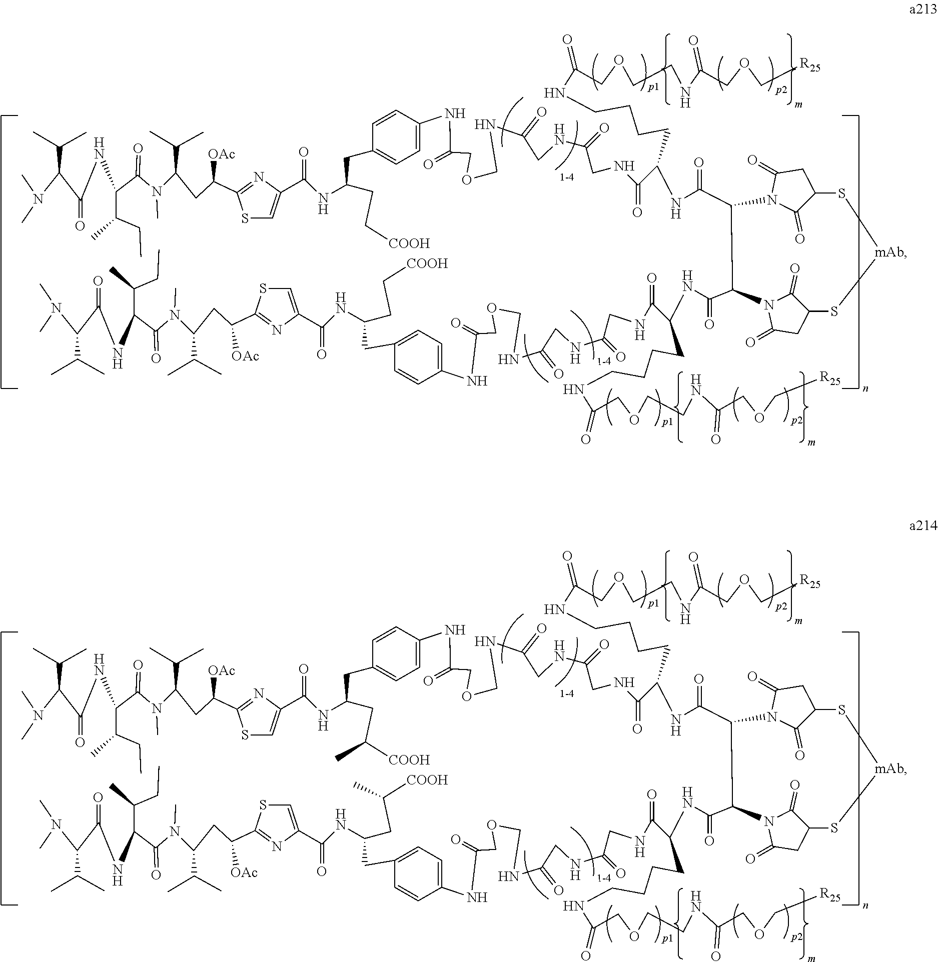 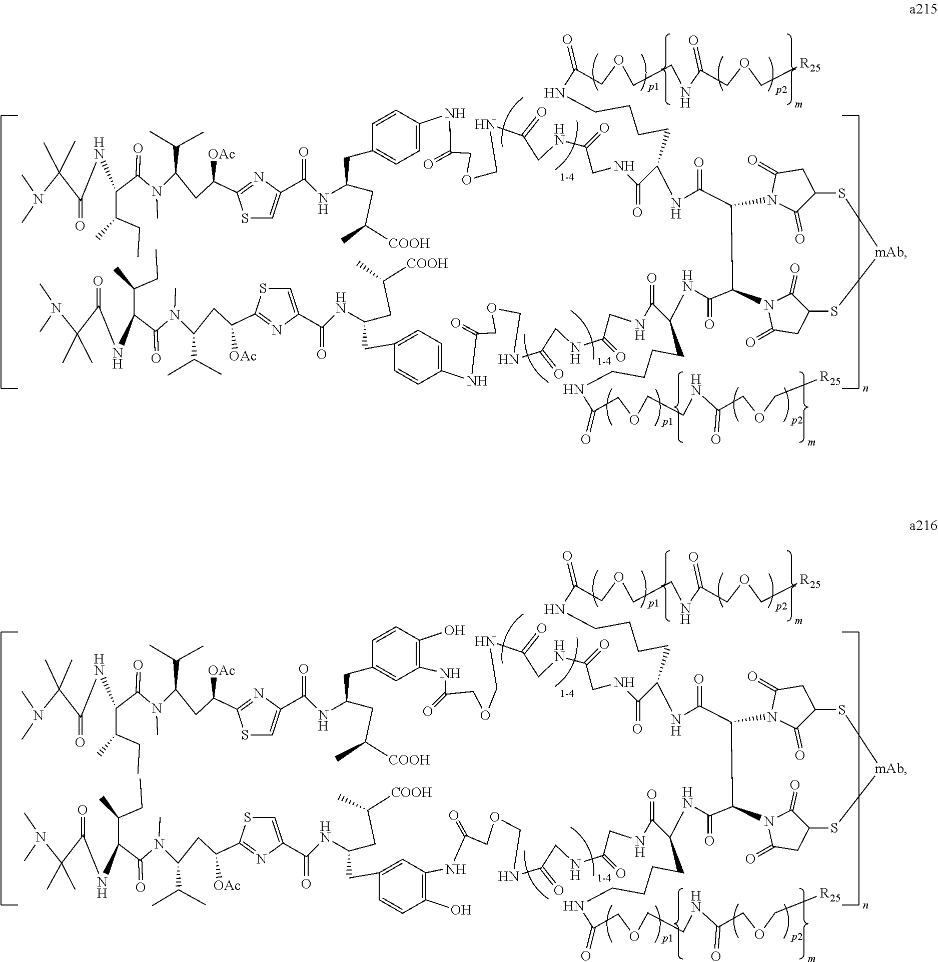 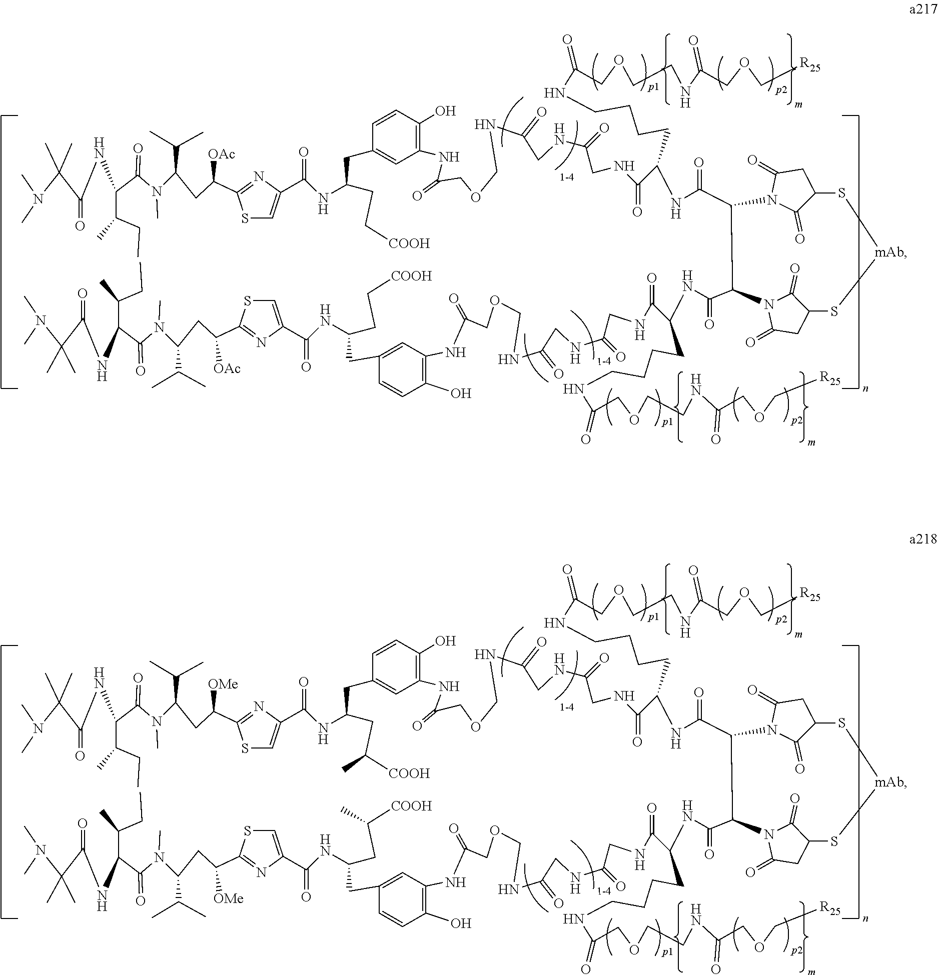 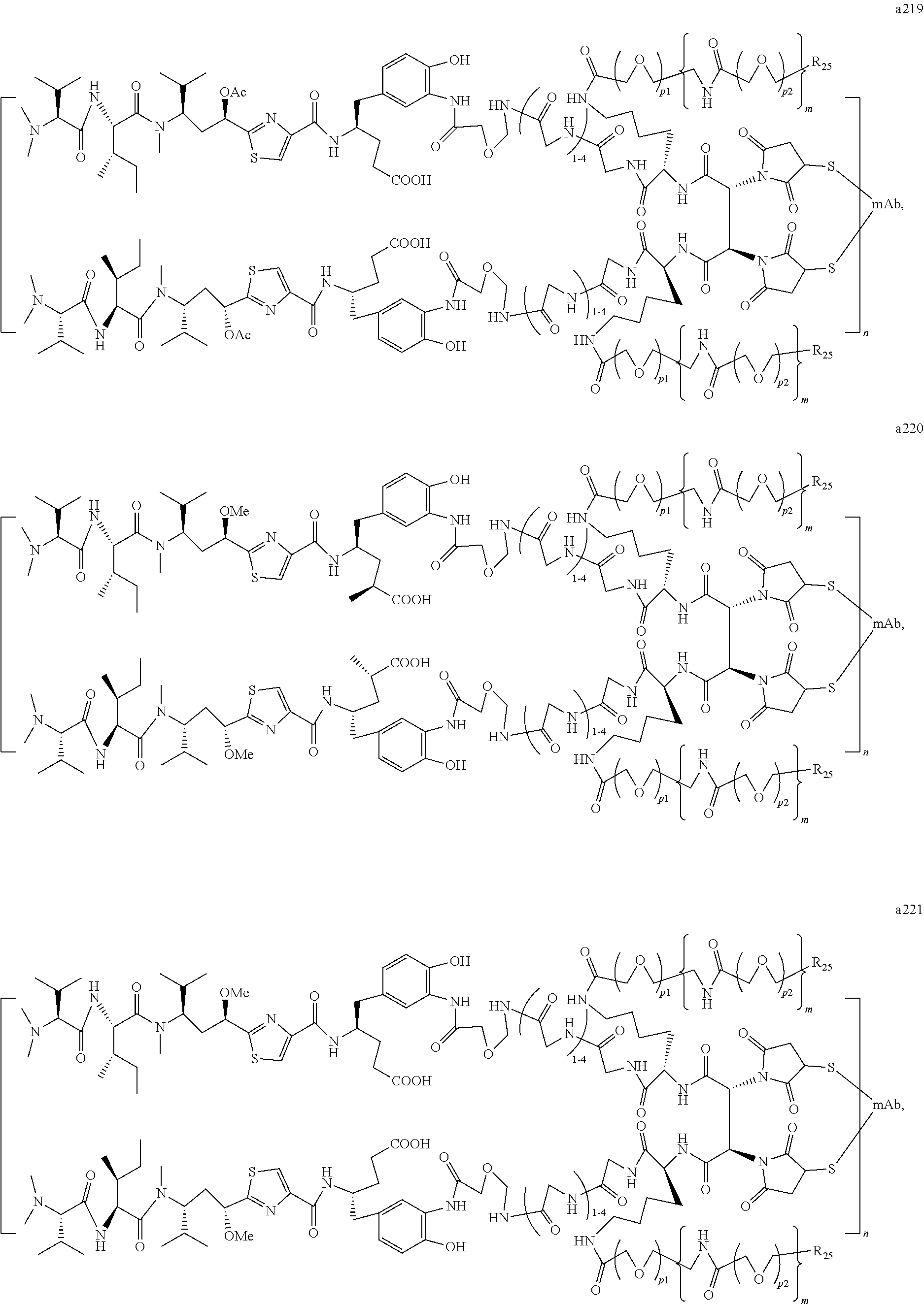 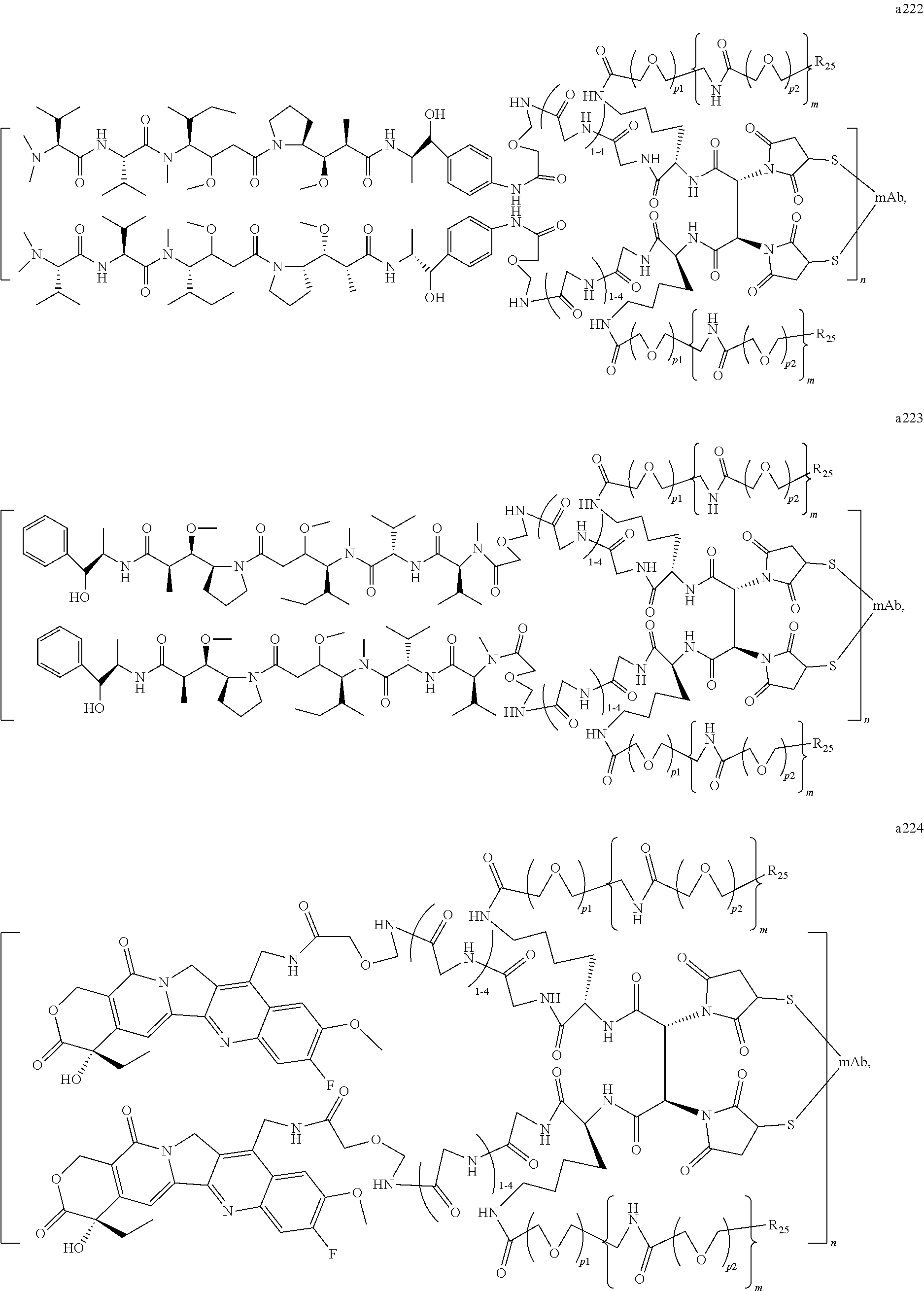 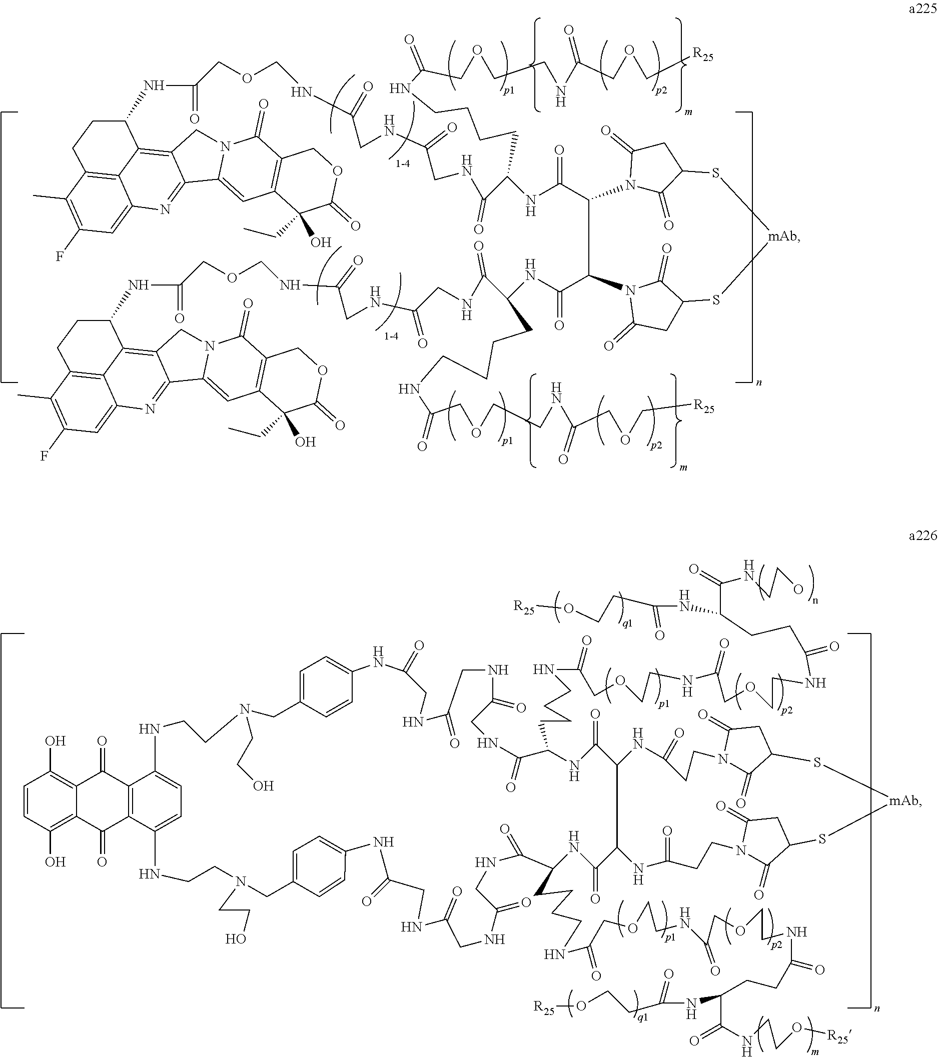 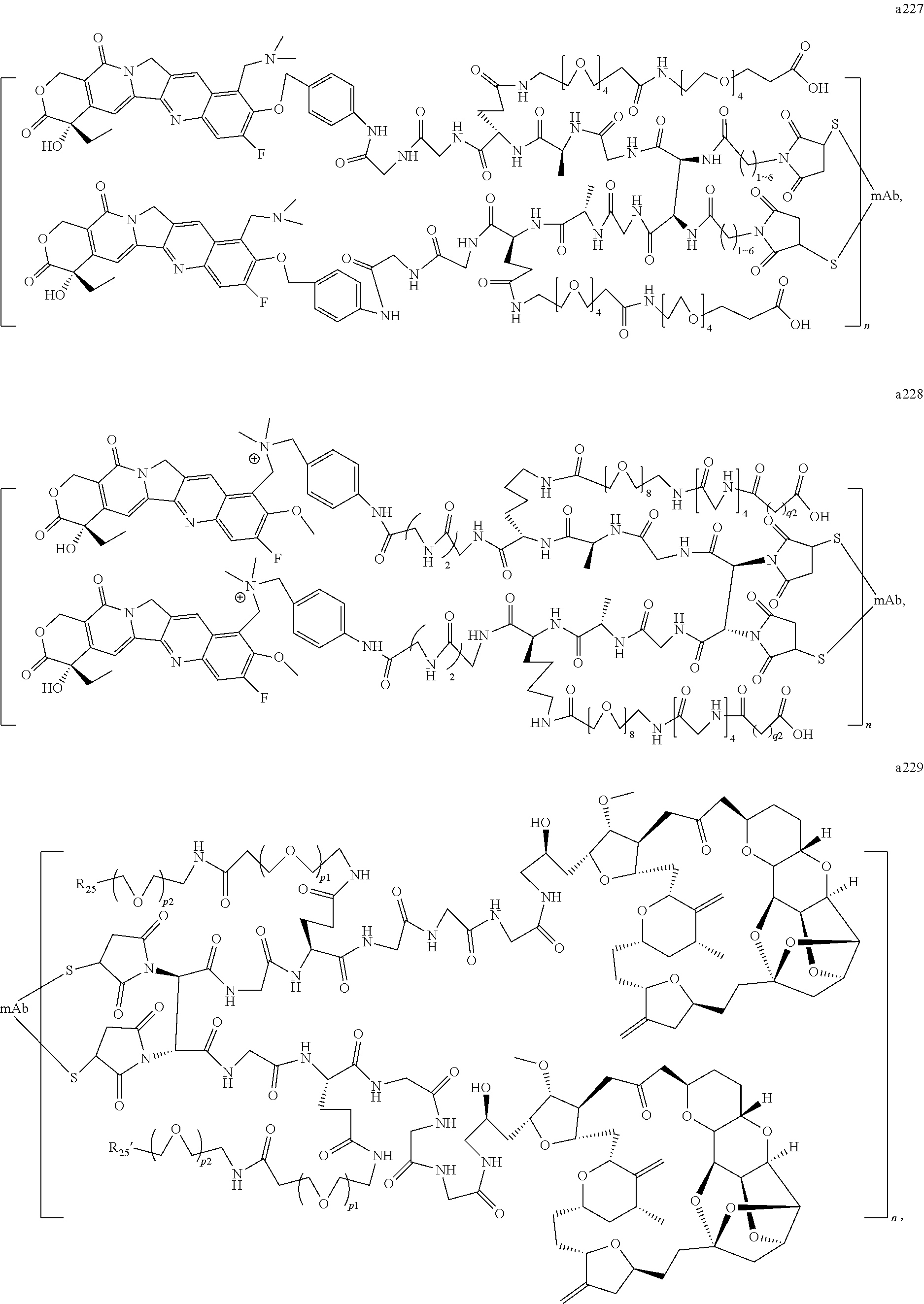 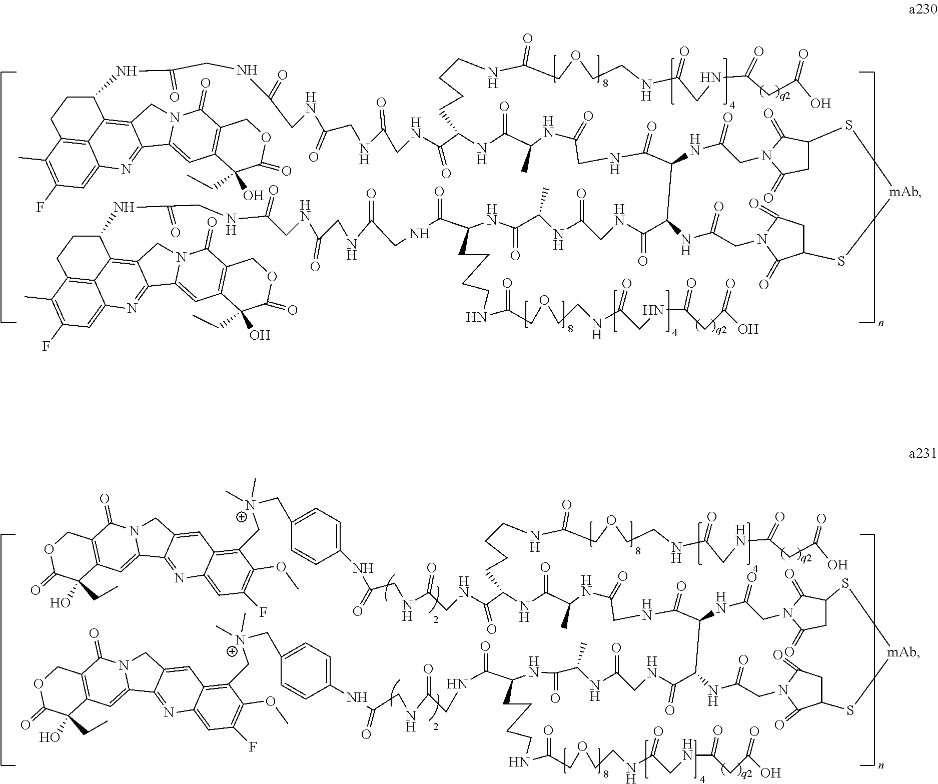 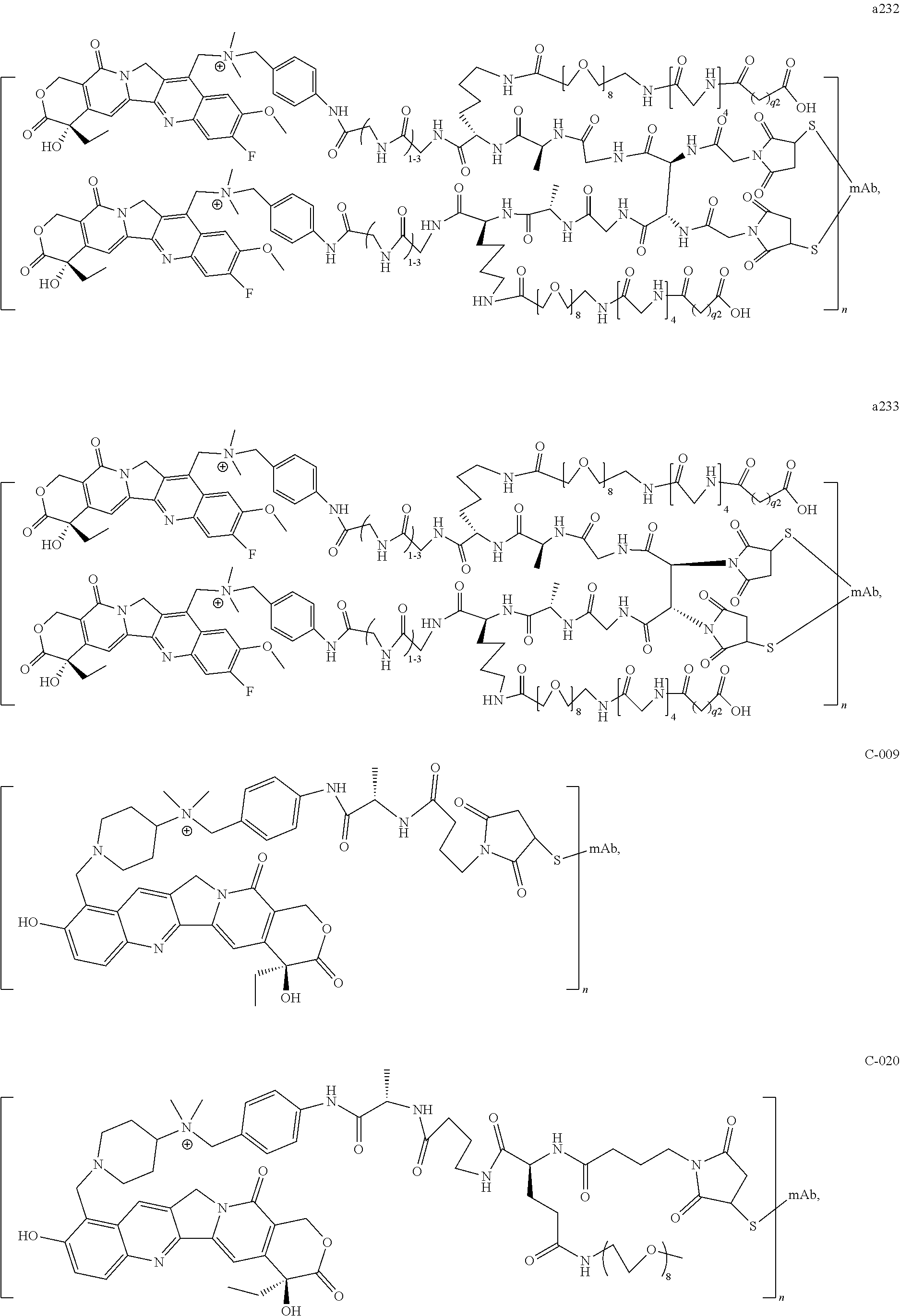 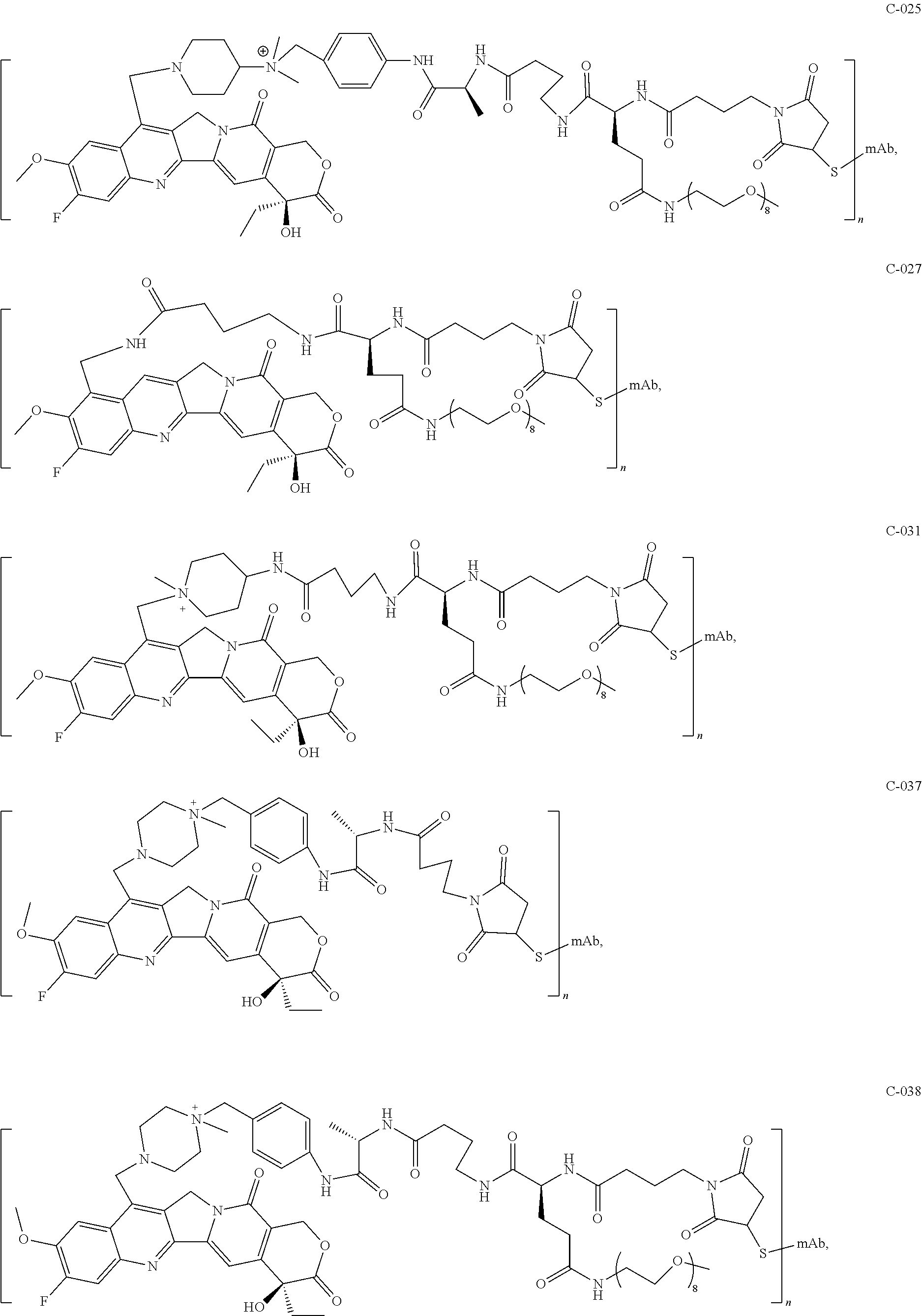 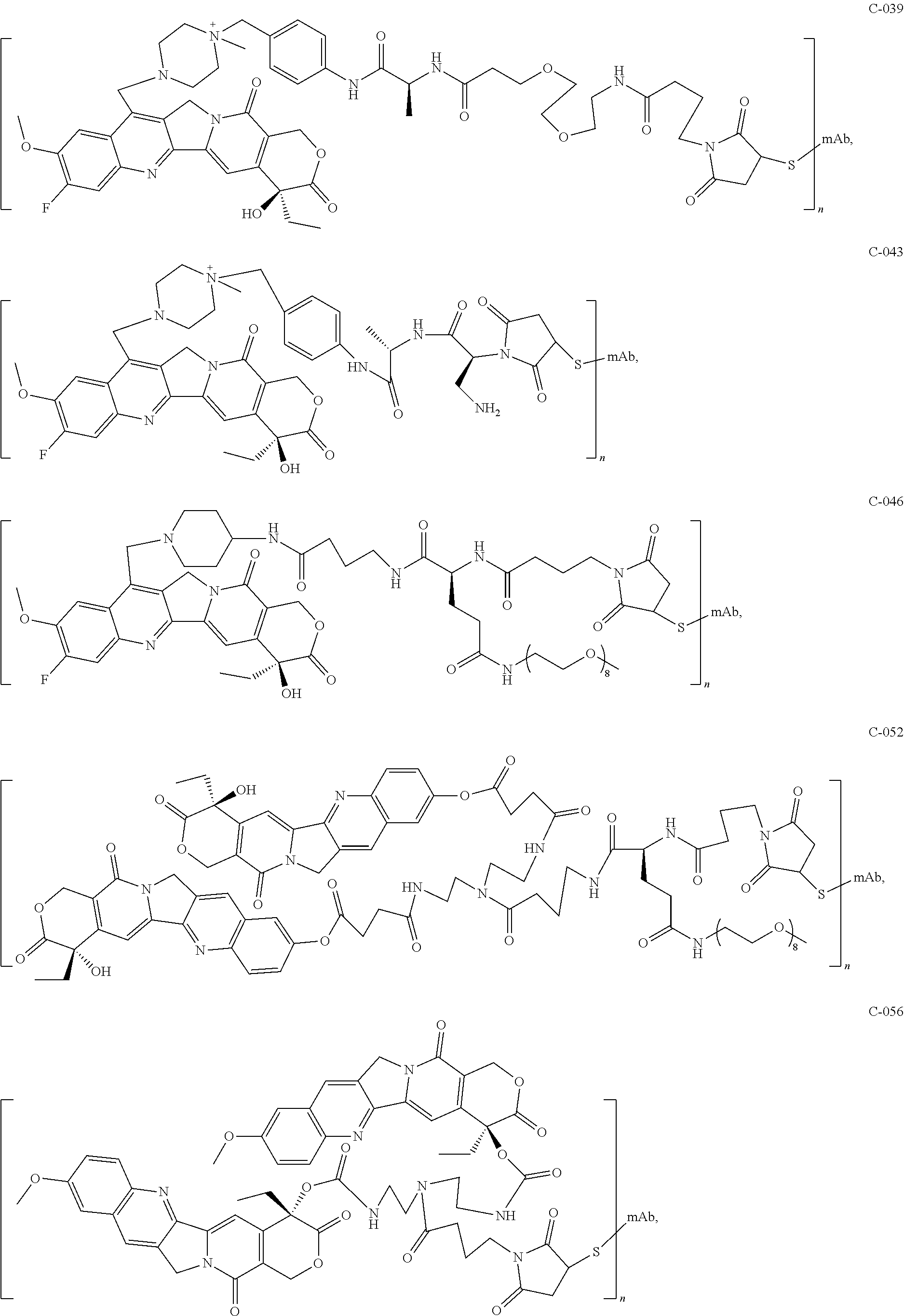 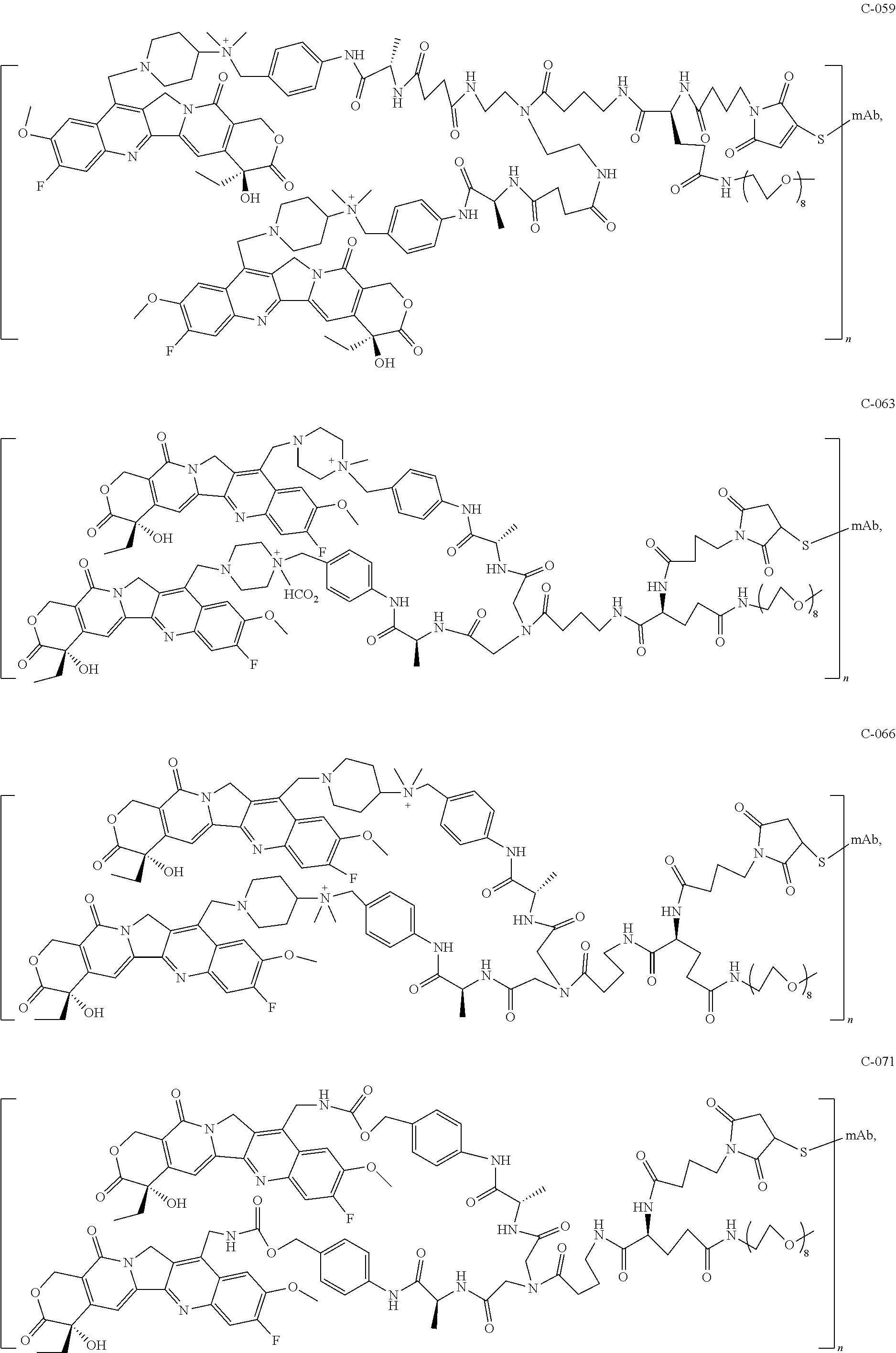 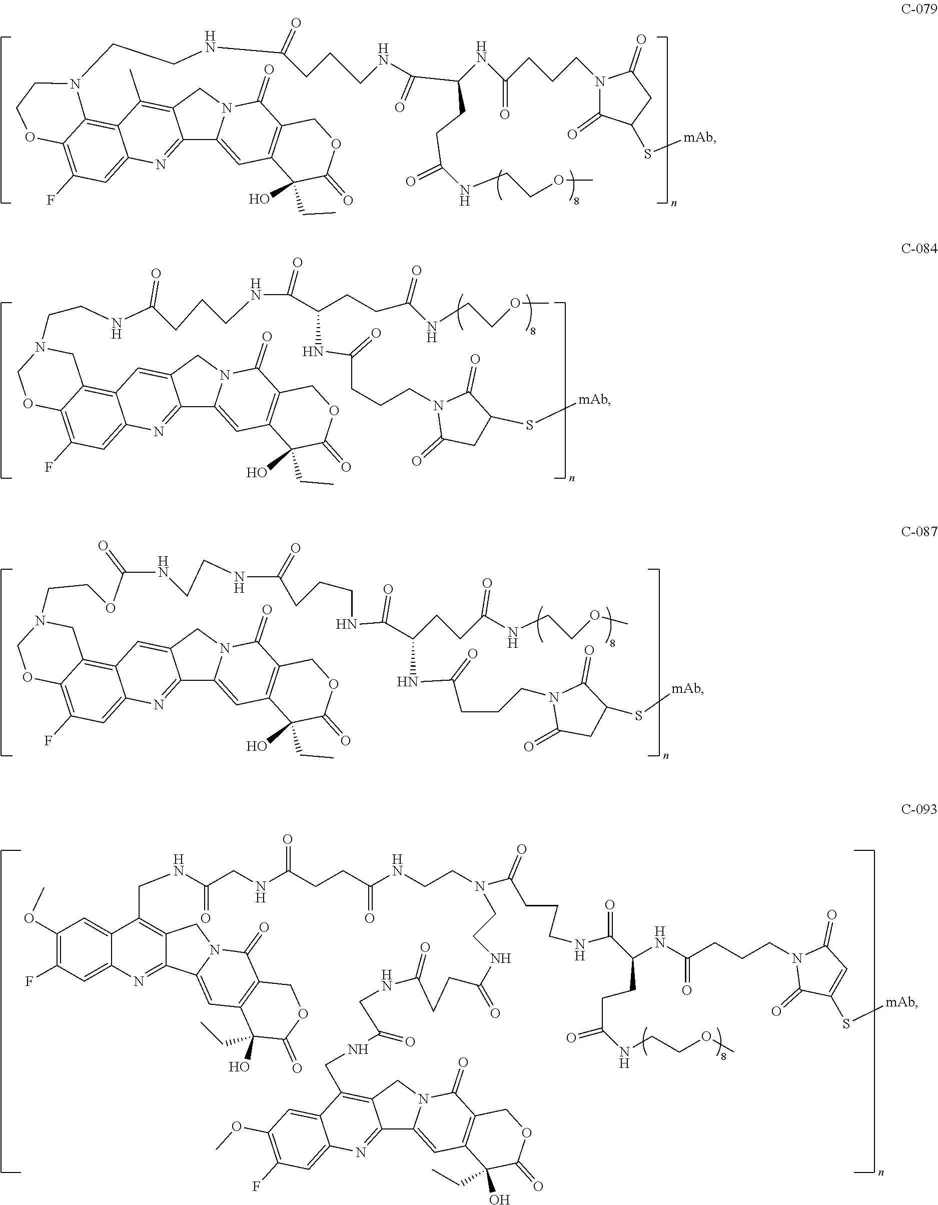 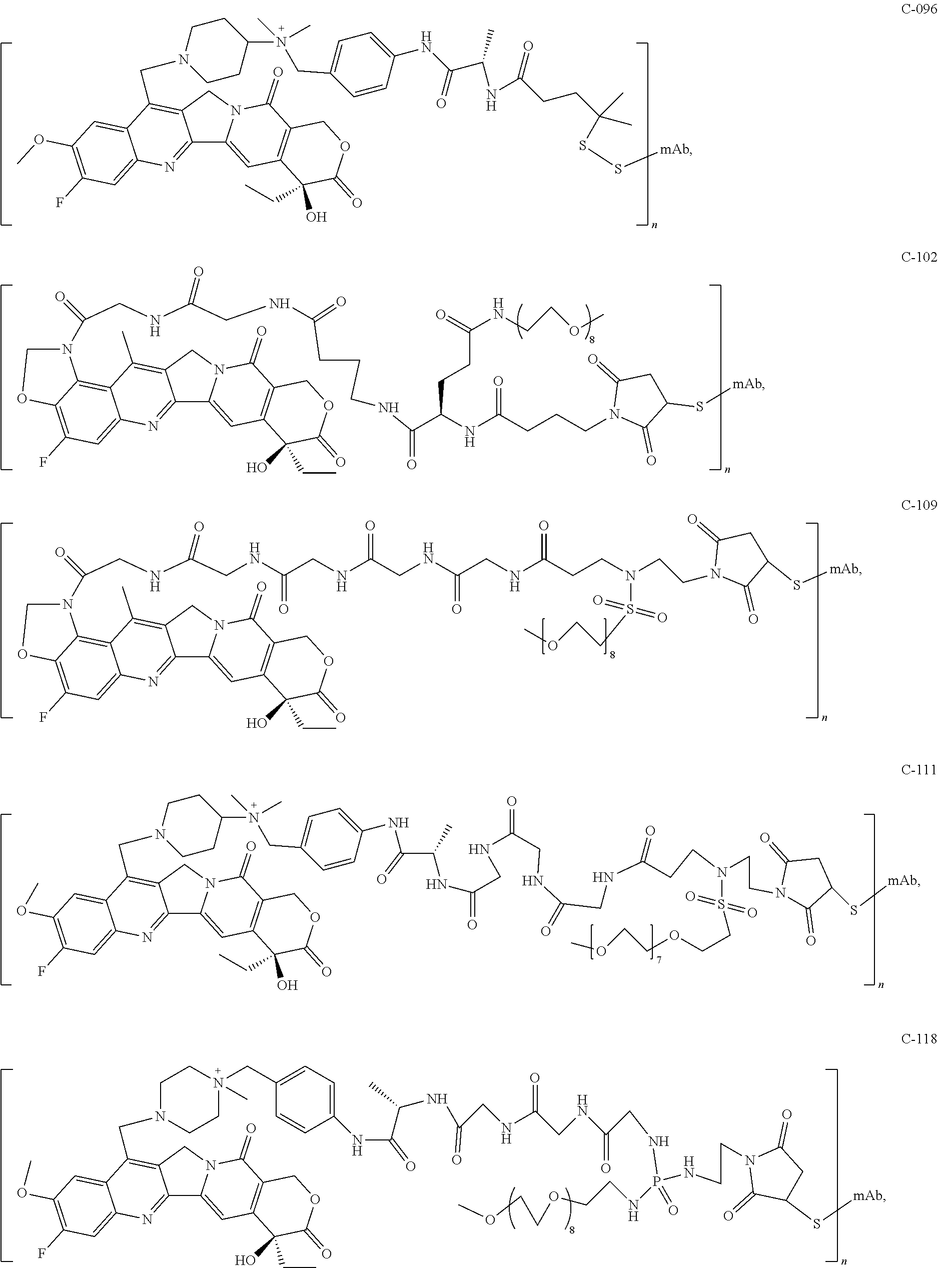 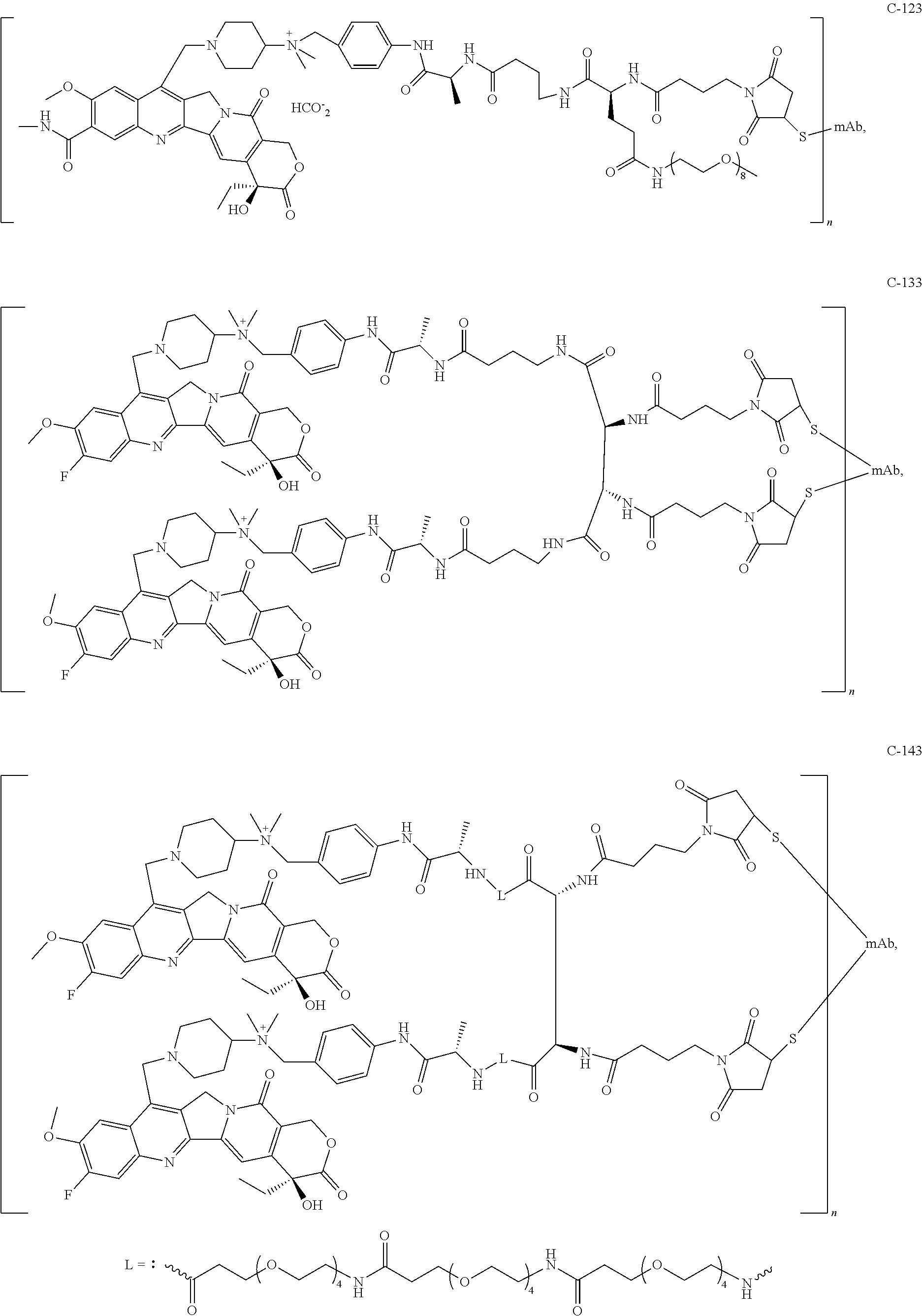 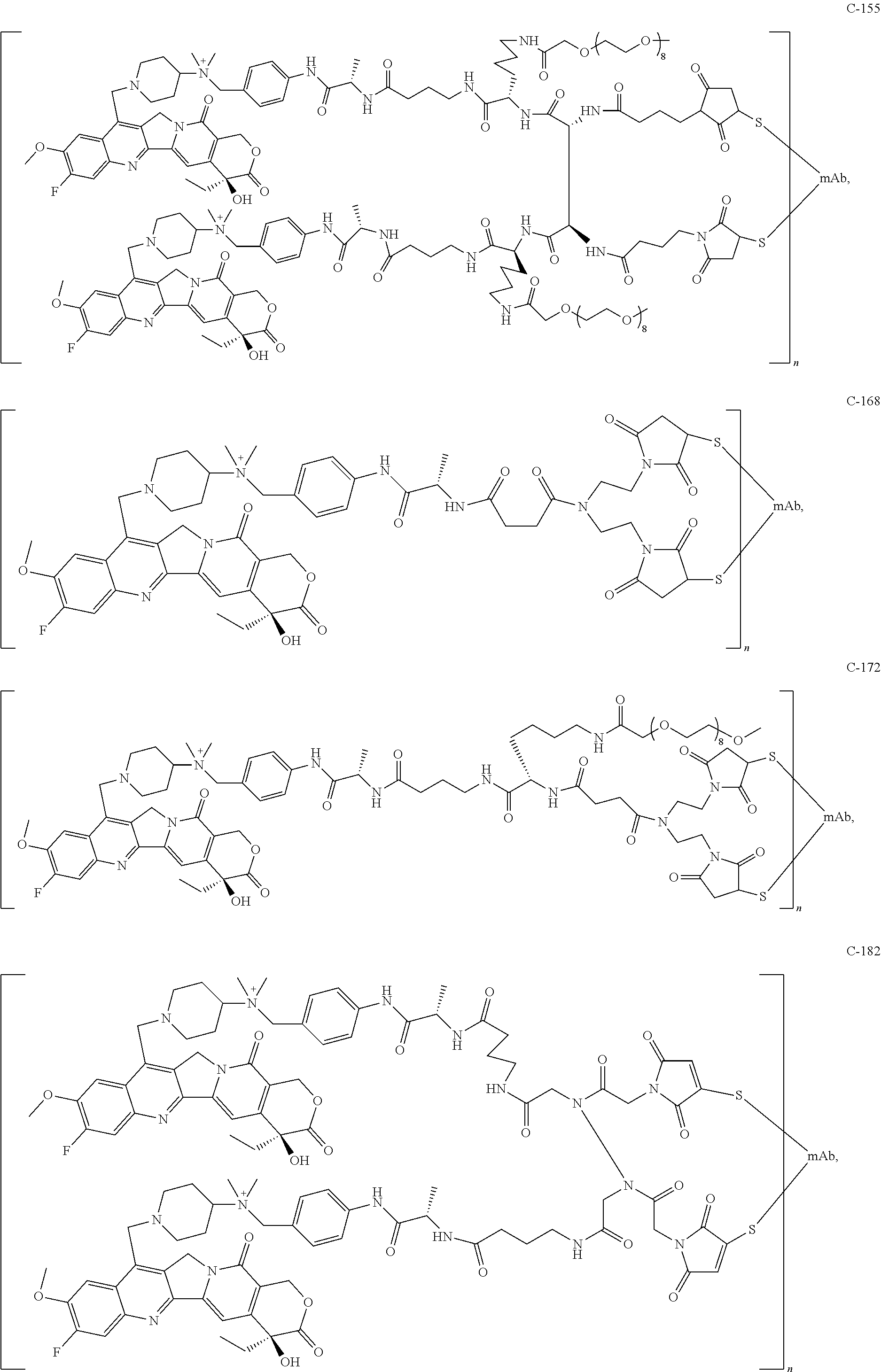 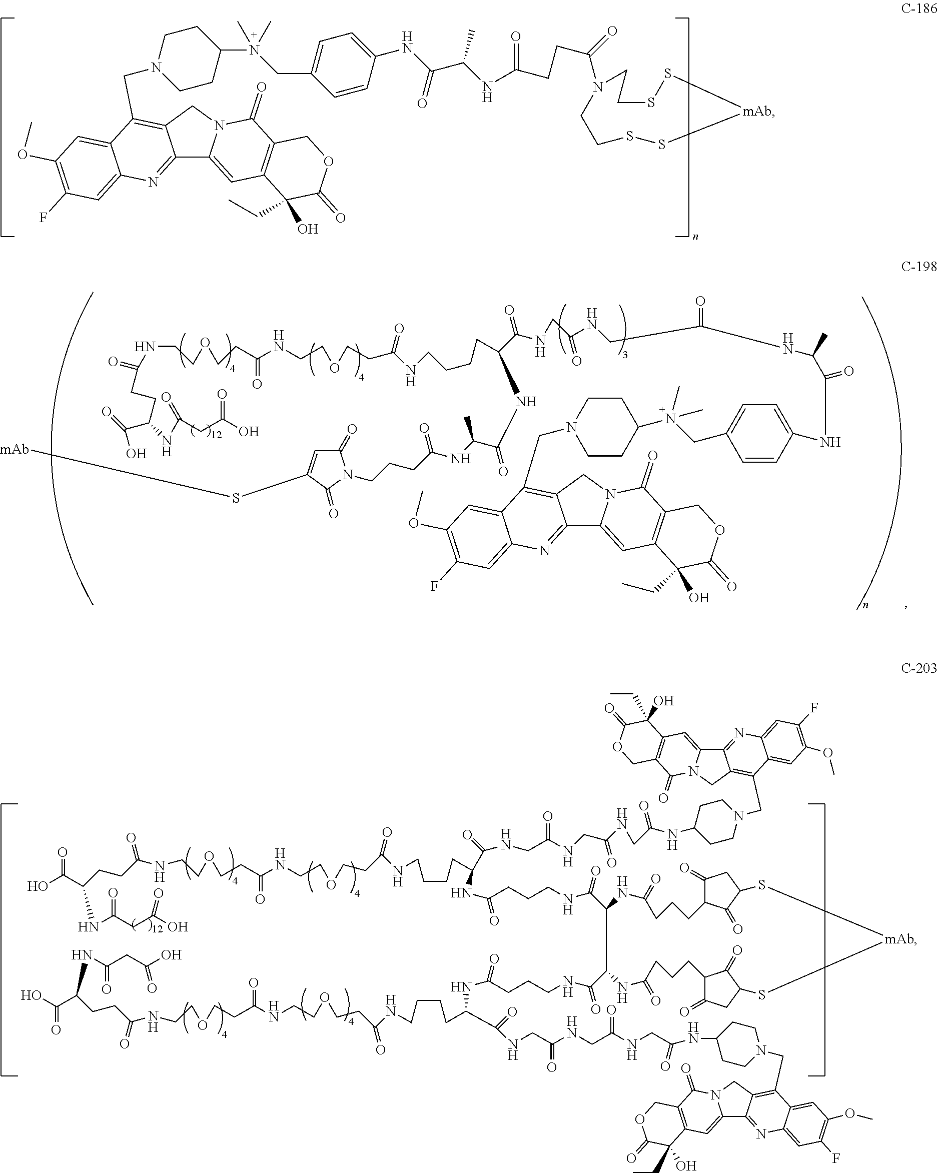 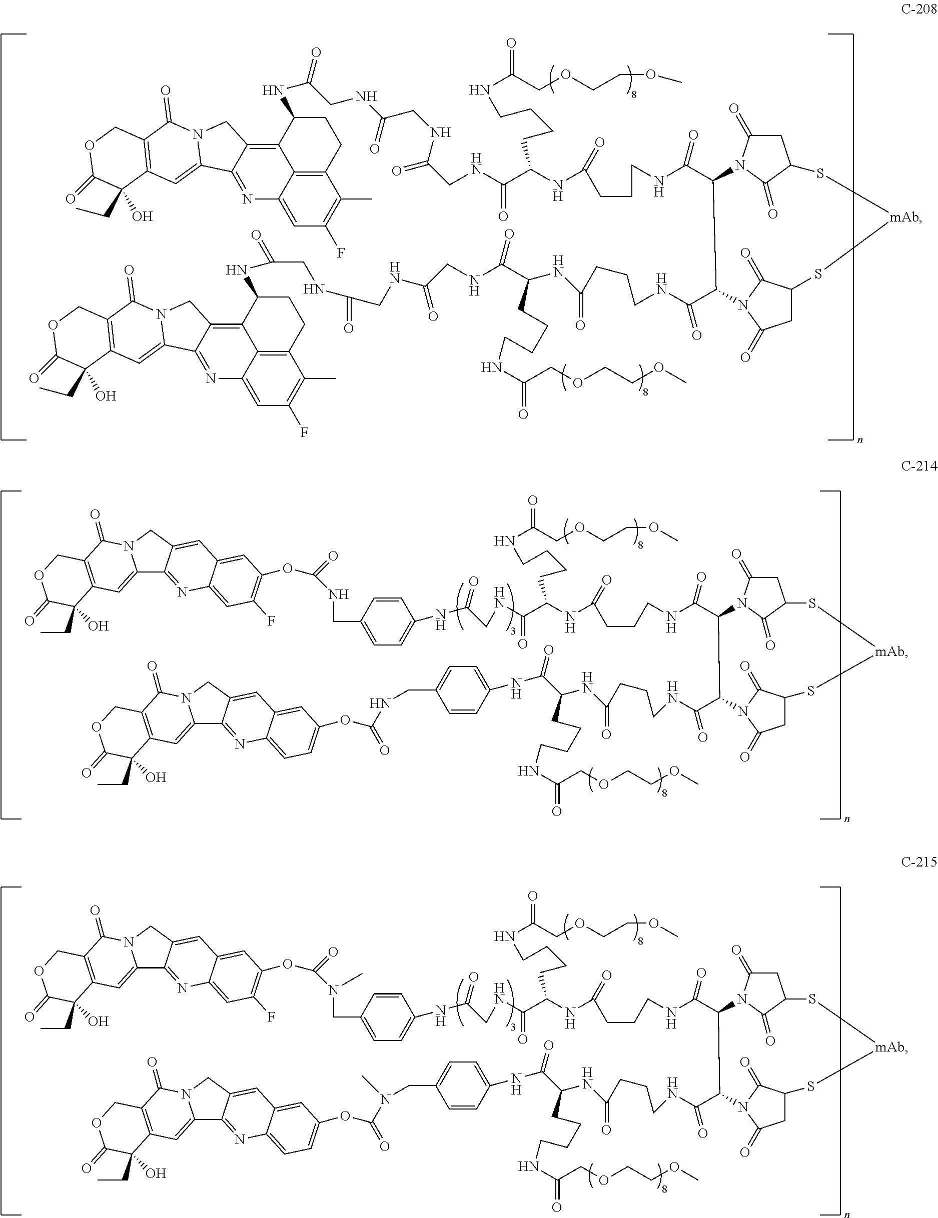 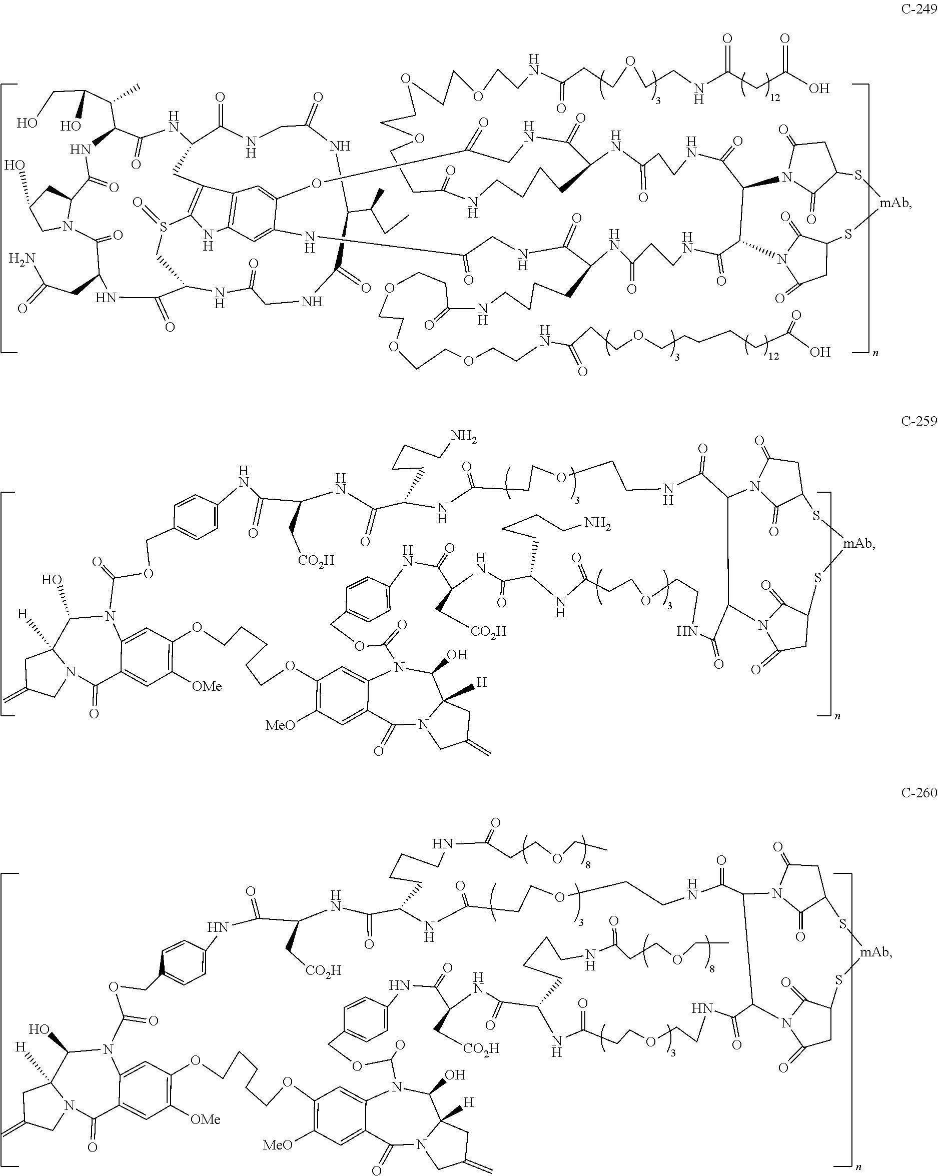 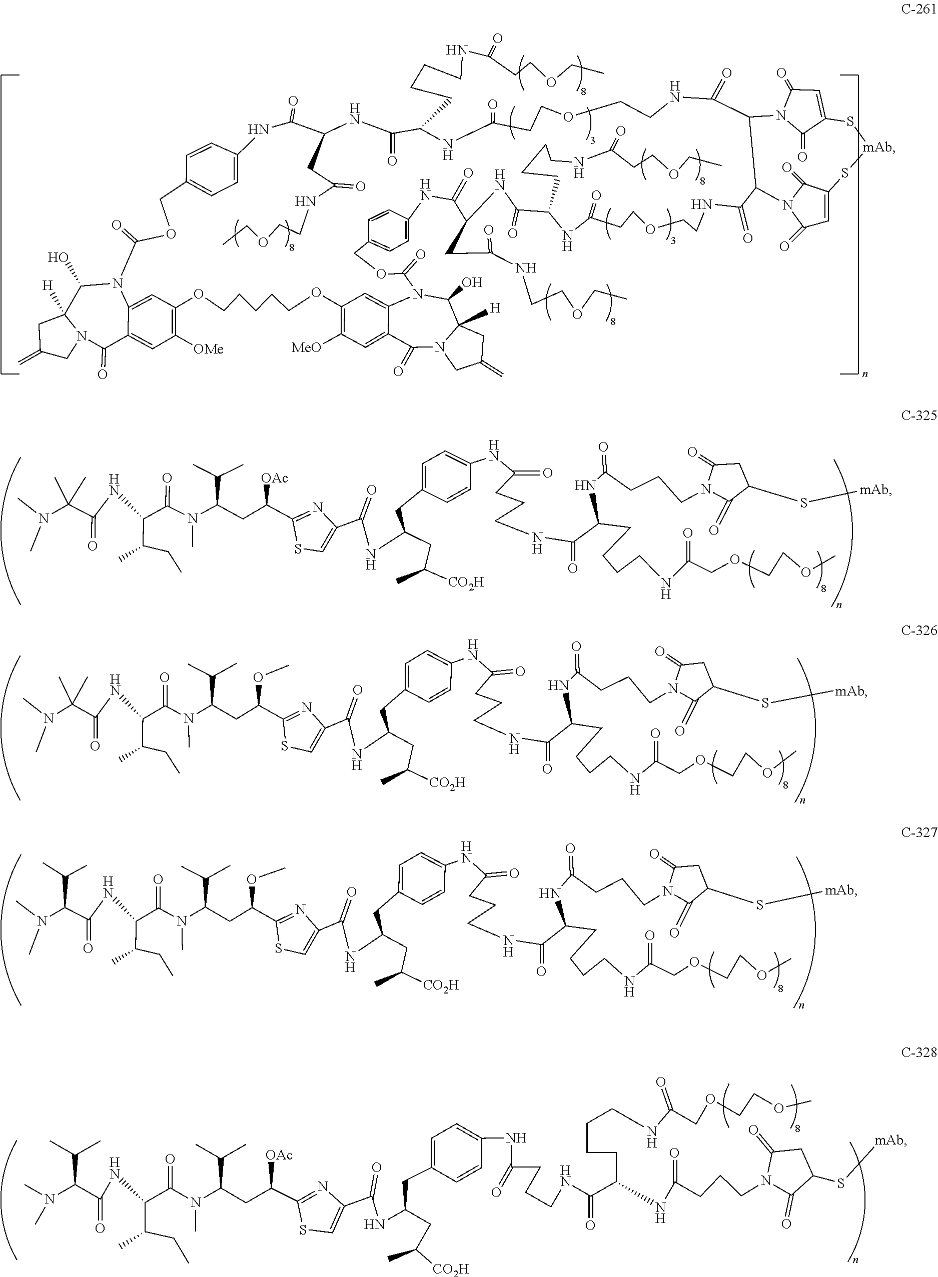 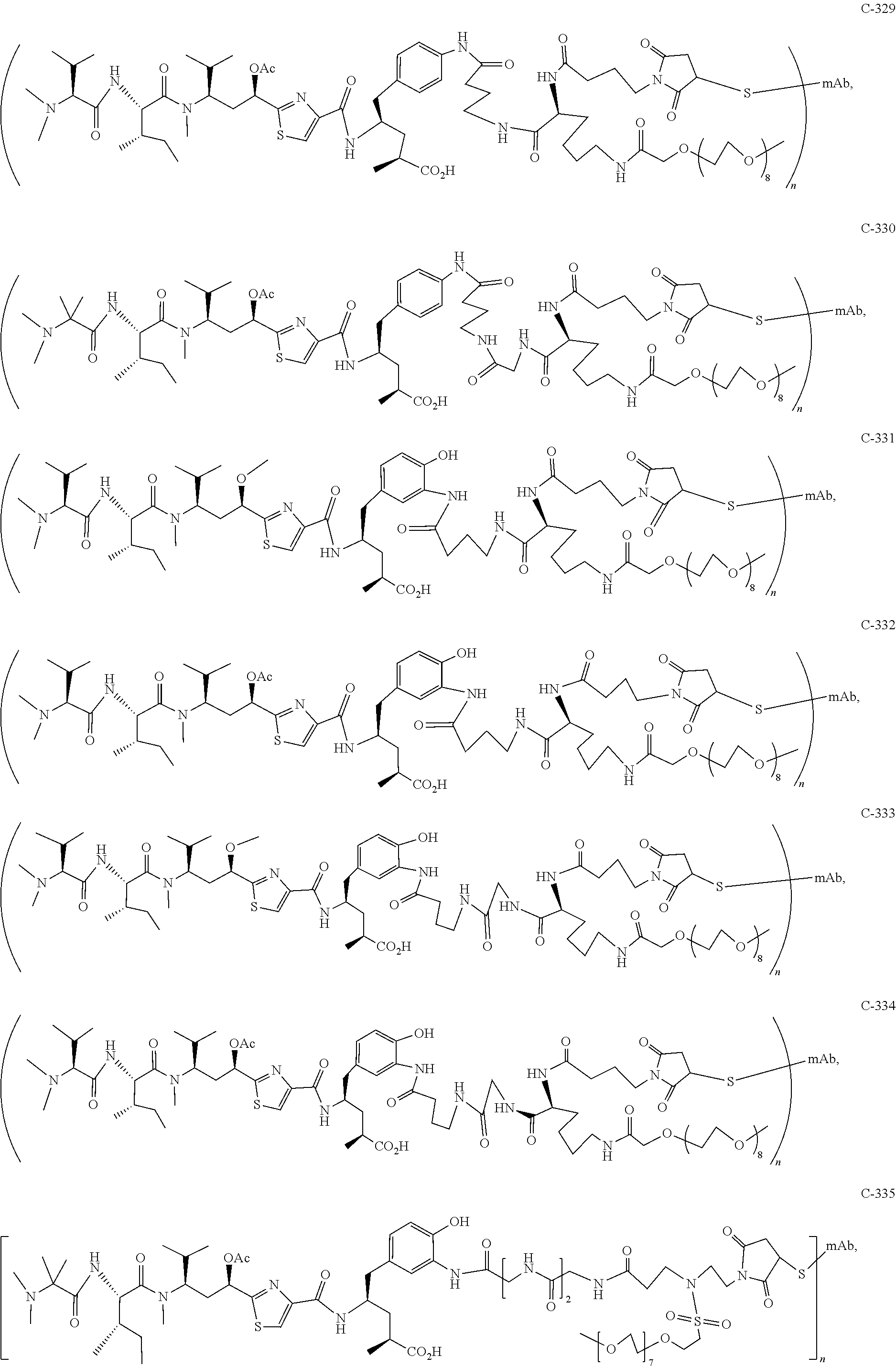 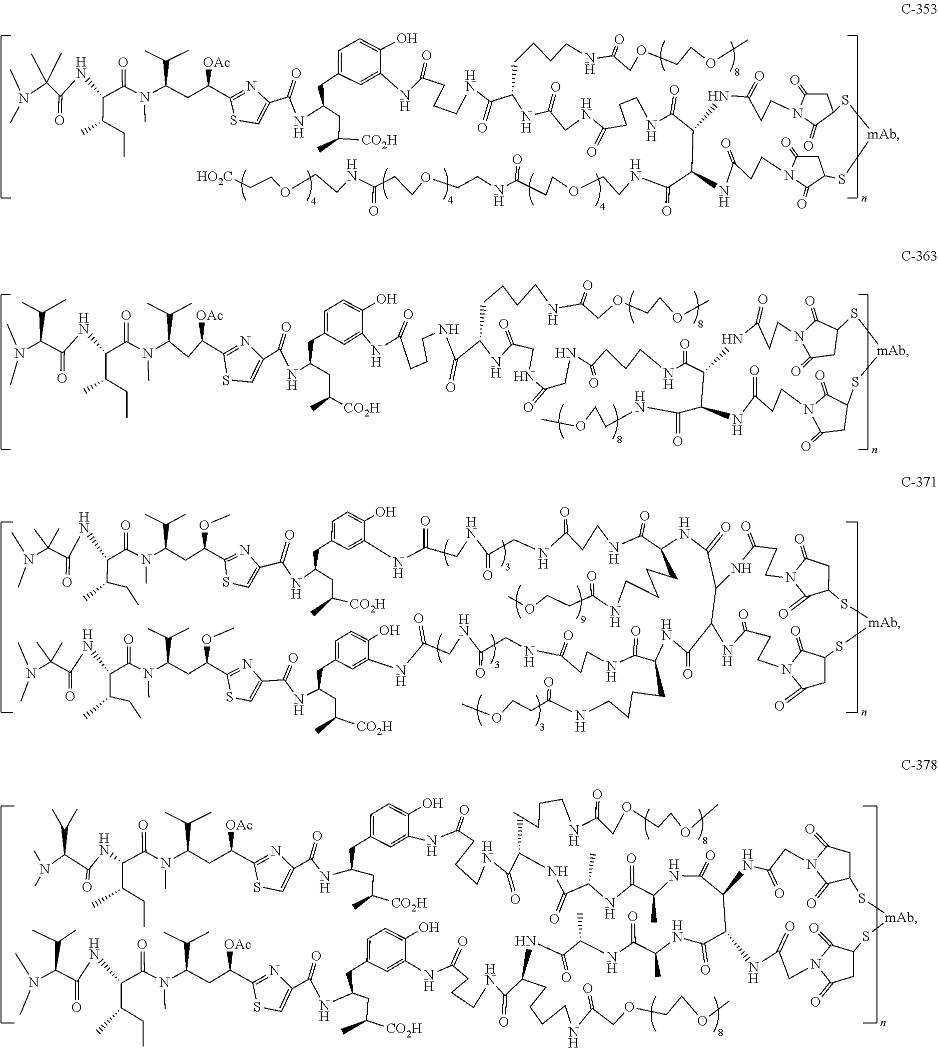 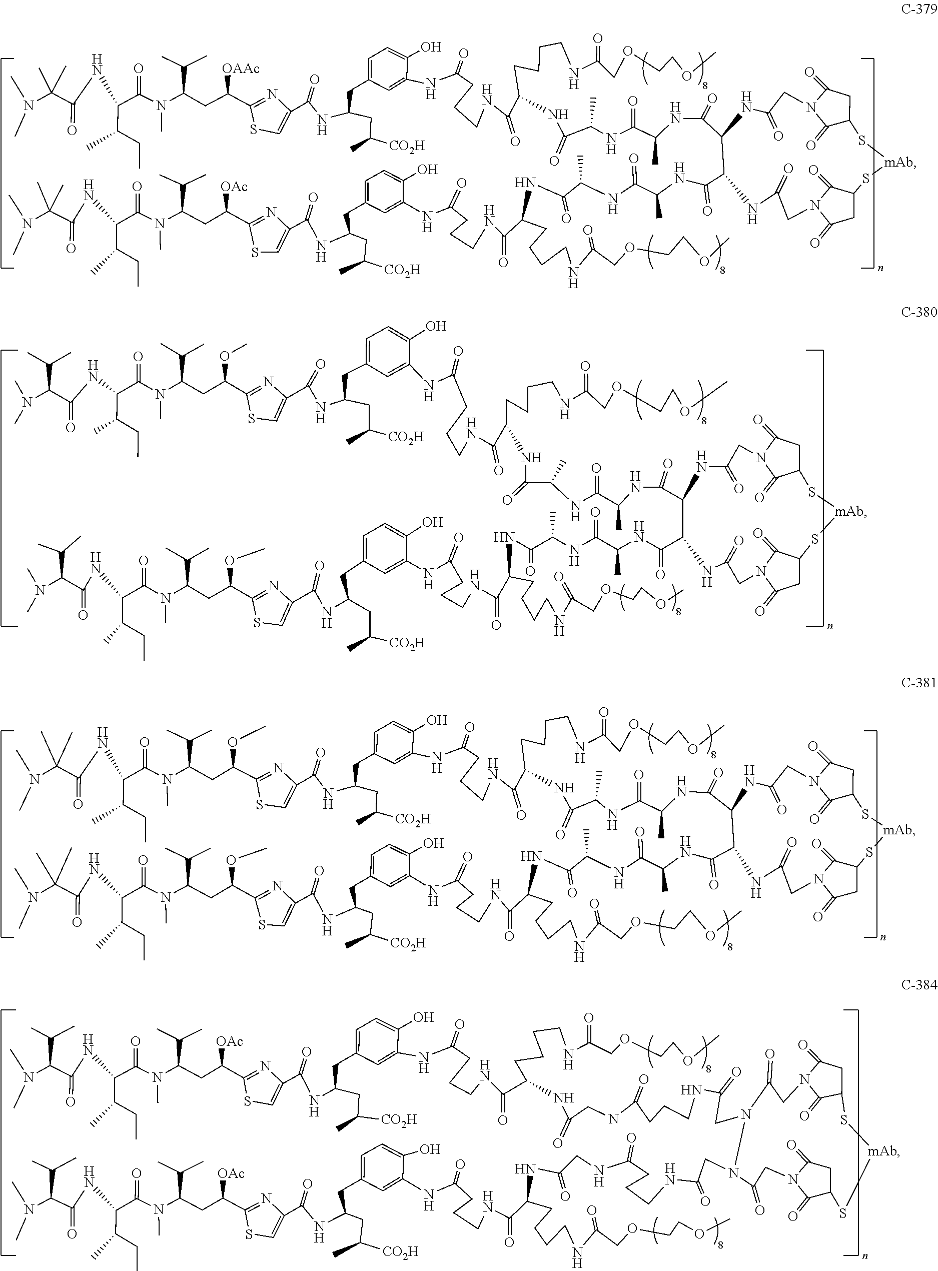 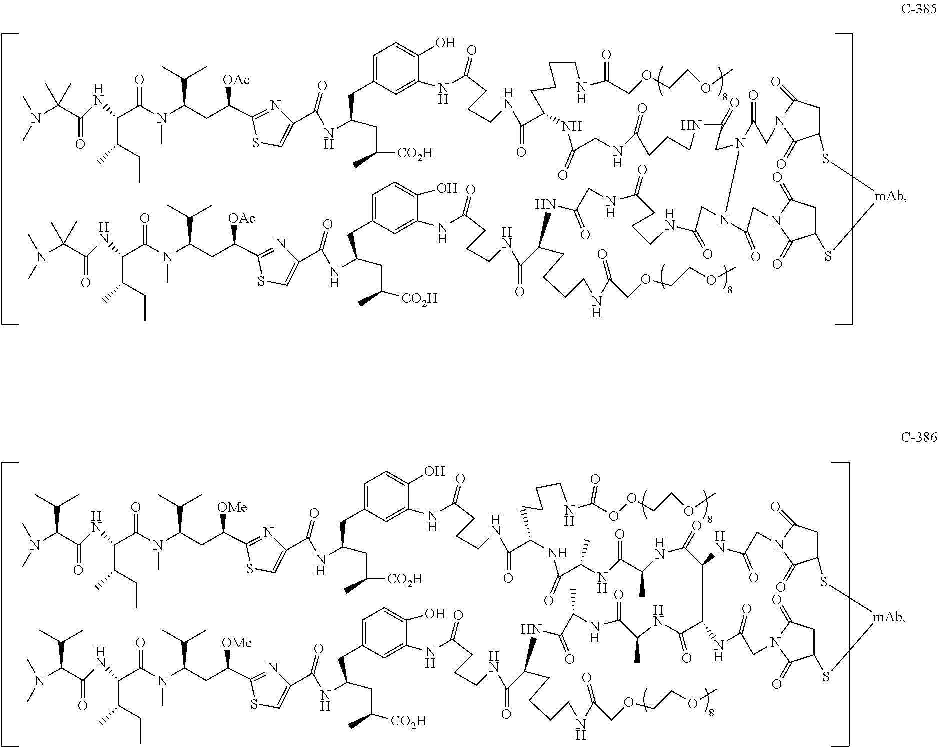 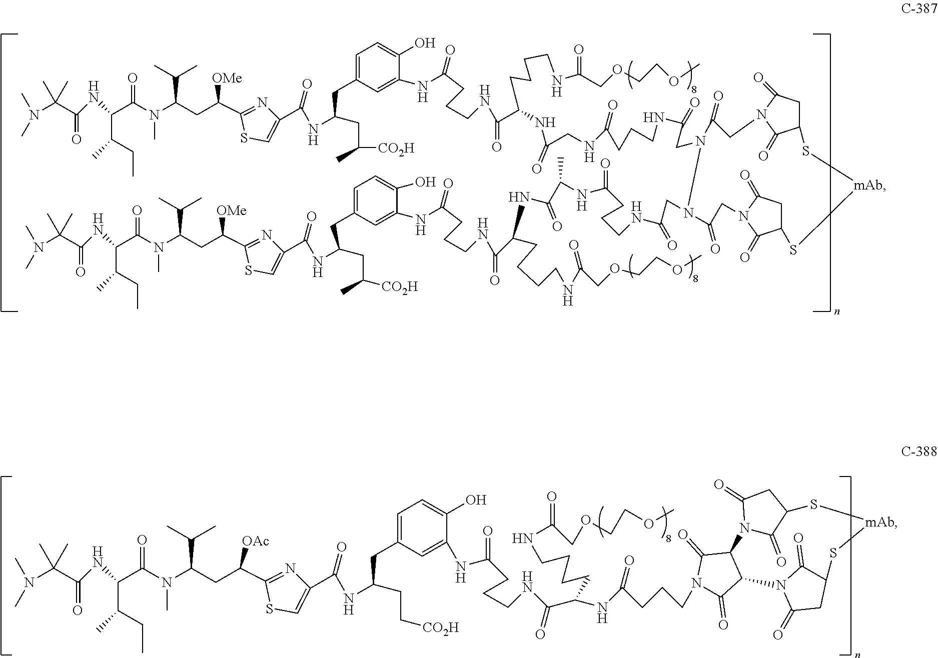 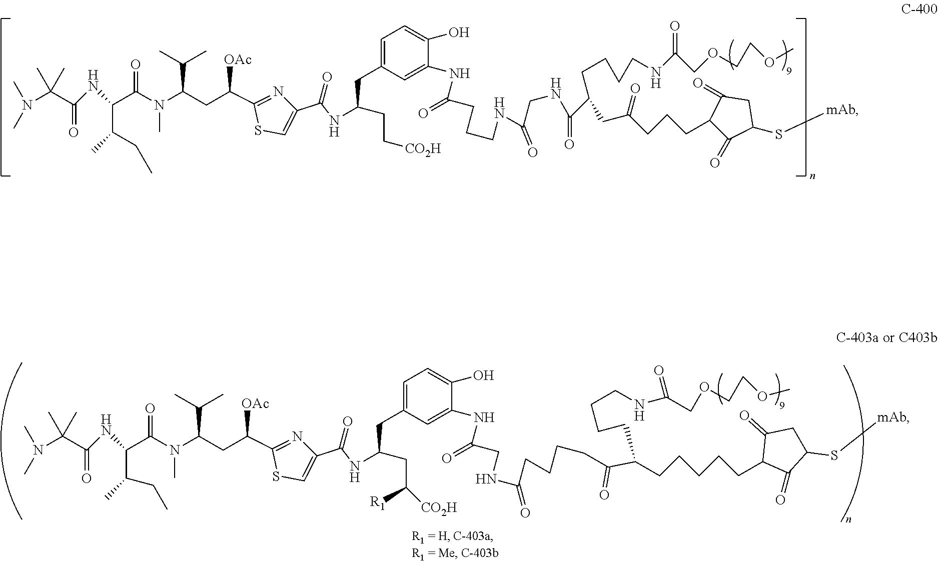 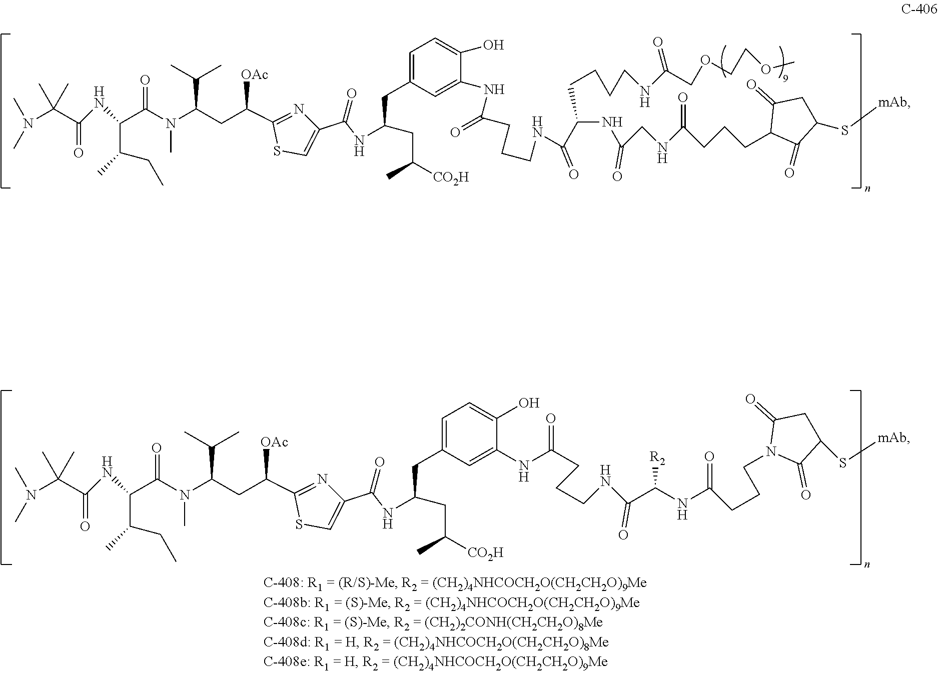 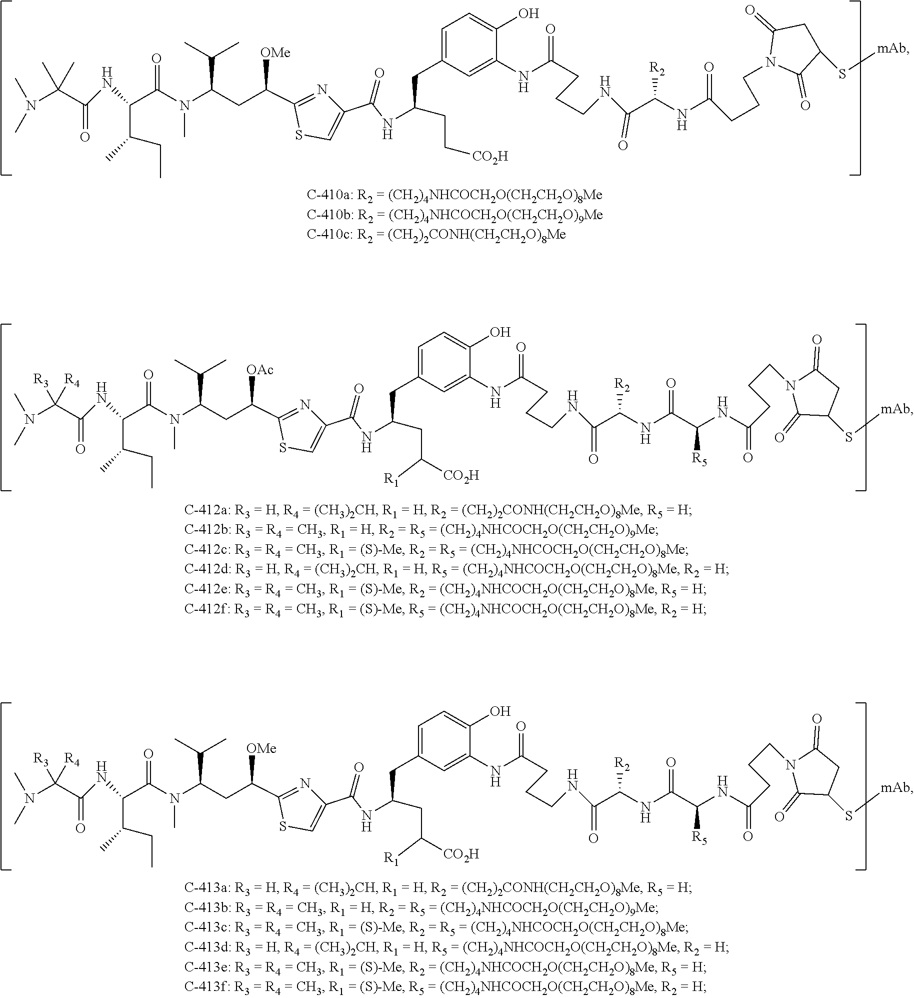 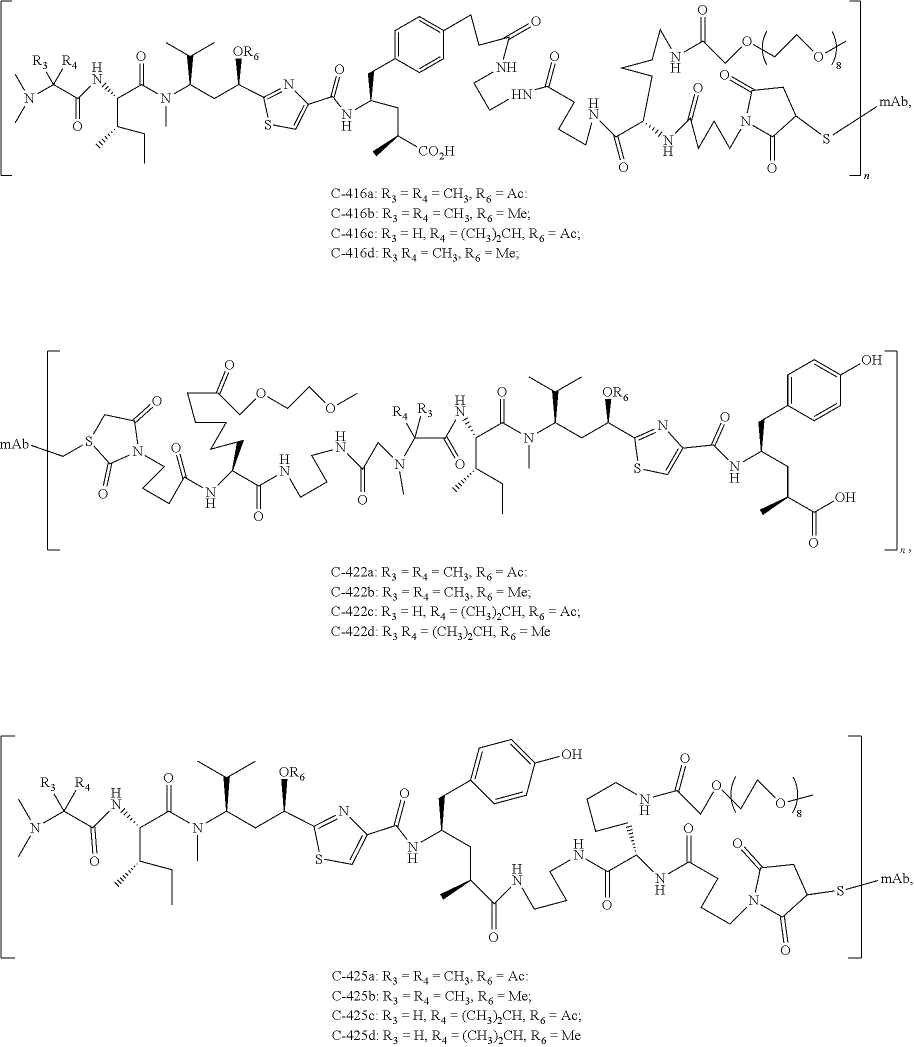 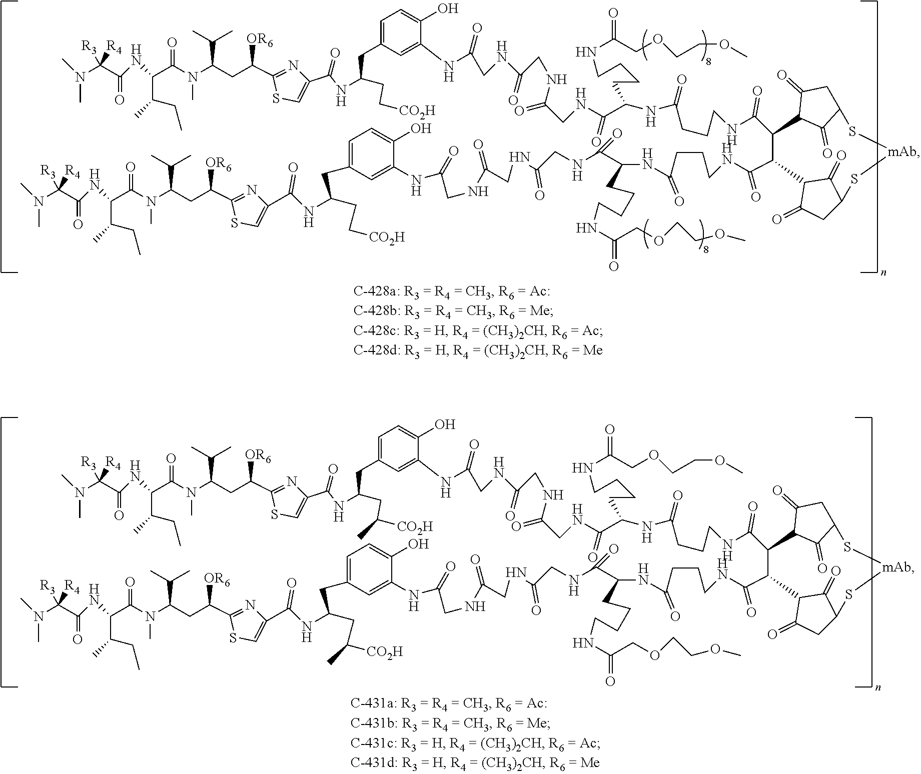
or a derivative with one or more isotopes, a pharmaceutically acceptable salt, hydrate, or hydrated salt thereof; or a polymorphic crystalline structure thereof; or an optical isomer, racemate, diastereomer or enantiomer thereof;
wherein p, p₁, p₂, and p₃ are independently 0-100; m, m₁, and m₂ are independently 0-20; n is 1-10;
P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR^{1′}, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); R¹⁷ and R¹⁸ are independently H, C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), or carbamate (—C(O)NR¹⁷R₈);
R¹, R², R³, R^{1′}, R^{2′}, R^{3′}, and R⁴ are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl; or R¹ and R², R¹ and R³, R² and R³, R³ and R⁴, R^{1′} and R^{2′}, R^{1′} and R^{3′} or R^{2′} and R^{3′} form a 3-7 membered carbocyclic, cycloalkyl, heterocyclic, heterocycloalkyl, aromatic or heteroaromatic ring system;
R₄, R₅, R₅′, and R₆ are independently H, C₁-C₈ linear or branched alkyl, C₃-C₈ aryl, heteroaryl, heteroalkyl, alkylcycloalkyl, or acyloxylamines, or (Aa)r, r=1-6 (amino acid or peptides having the same or different sequence of amino acids);
R₇, R₈, and R₉ are independently H, OH, OR₁, NH₂, NHR₁, C₁-C₆ alkyl, or absent;
R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent;
R₁₁ is OH, NH₂, NHR₁, NHNH₂, NHNHCOOH, O—R₁—COOH, NH—R₁—COOH, NH—(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa)ᵣ is 1-8 aminoacids; n and m₁ are independently 1-20; p is 1-1000; R₁, R₂ and Ar, are the same defined above; “ [symbol] ” is defined the same above;
R₁₂ and R₁₂′ are independently H, ═O, OR₁, NH₂, NH(CH₃), NHNH₂, COOH, SH, OZ₃, SZ₃, F, Cl, C₁-C₈ of linear or branched alkyl, or C₃-C₈ aryl, heteroaryl, heteroalkyl, alkylcycloalkyl, or acyloxylamines;
R₂₅, R₂₆ and R₂₅′ are are independently H, Ac, R₁, C(O)NHR₁, C(O)R₁, R₁COOH, R₁COOR₂, R₁OR₂, R₁CONHR₂, CH₂OAc, CH₂NHAc, R₁NH₂, NR₁R₂, N⁺R₁R₂R₃, CH₂CONH(CH₂)_{q1}COOH, CH₂CONH(CH₂)_{q1}COOR₁, CH₂CONH(CH₂)_{q1}N⁺R₁R₂R₃, or (Aa)r;
X₁, X₂, X₄, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), CH₂, CHNH, CH₂O, C(O)NHNHC(O), OCH₂C₆H₄NH, NHCH₂C₆H₄NH, SCH₂C₆H₄NH or C(O)NR₁ when linked to a connecting site“ [symbol] ”; or OH, NH₂, NHNH2, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH or C(O)NHR₁ when not linked to the connecting site “ [symbol] ”; Y₂ can further be 0, 02, NR₁, NH, or absent when it links S; [symbol]
X₃ and Y₃ are independently N, NH, CH, CH₂ or CR₁, or one of X₃ and Y₃ can be absent;
wherein R₁ is C₁-C₈ linear or branched alkyl, or heteroalkyl; C₃-C₈ aryl, heteroaryl, alkylcycloalkyl, acyloxyl, alkylaryl, alkylaryloxyl, alkylarylamino, or alkylarylthiol; (Aa)r, r=1-6 (amino acid or peptide having the same or different sequence of amino acids);
Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃;
Z₃ is H, OP(O)(OM₁)(OM₂), OCH₂OP(O)(OM₁)(OM₂), OSO₃M₁, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃;
X₅ is F, Cl, Br, I, OH, OR₁, R₁, OPO₃H₂, OSO₃H, NHR₁, OCOR₁, NHCOR₁, CN or OCH₂OP(O)(OM₁)(OM₂);
Y₅ is NH, NHNH, NR₁, O, OCH₂C₆H₄NH, NHCH₂C₆H₄NH, SCH₂C₆H₄NH, R₁, (Aa)r, r=1-6 (amino acid or peptides having the same or different sequence of amino acids);
X₆ and Y₆ are independently CH, C(O), N, P(O)NH, P(O)NR₁, CHC(O)NH, C₁-C₈ linear or branched alkyl, or heteroalkyl; C₃-C₈ aryl, heteroaryl, alkylcycloalkyl, acyloxyl, alkylaryl, alkylaryloxyl, or alkylarylamino, or an Aa (amino acid);
Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) or C(O)NR₁;
X₈ is O, S, NH, NHNH, NHR₁, SR₁₂, SSR₁₂, SSCH(CH₃)R₁, SSC(CH₃)₂R₁, or R₁;
wherein R₁, R₂ and R₃ are in dependently H, C₁-C₈ linear or branched alkyl, C₃-C₈ aryl, heteroaryl, heteroalkyl, alkylcycloalkyl, or acyloxylamines unless they are defined in the structural formulae;
Lᵥ₁ is a leaving group the same as defined above;
M₁ and M₂ are independently H, Na, K, Ca, Mg, NH₄, or NR₁R₂R₃.

36. The process according to claim 23, wherein the drug/linker complex has one of the structures of below: 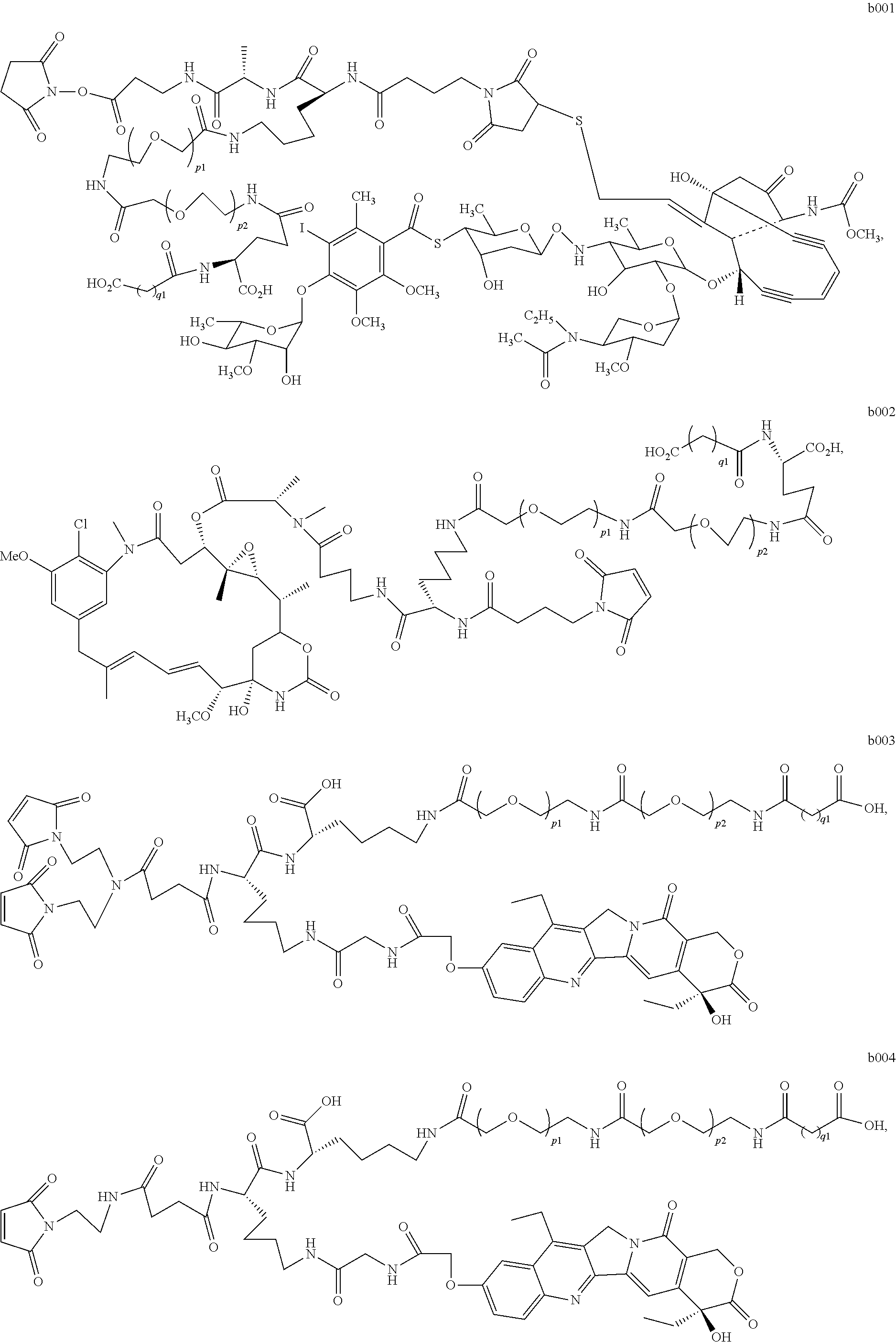 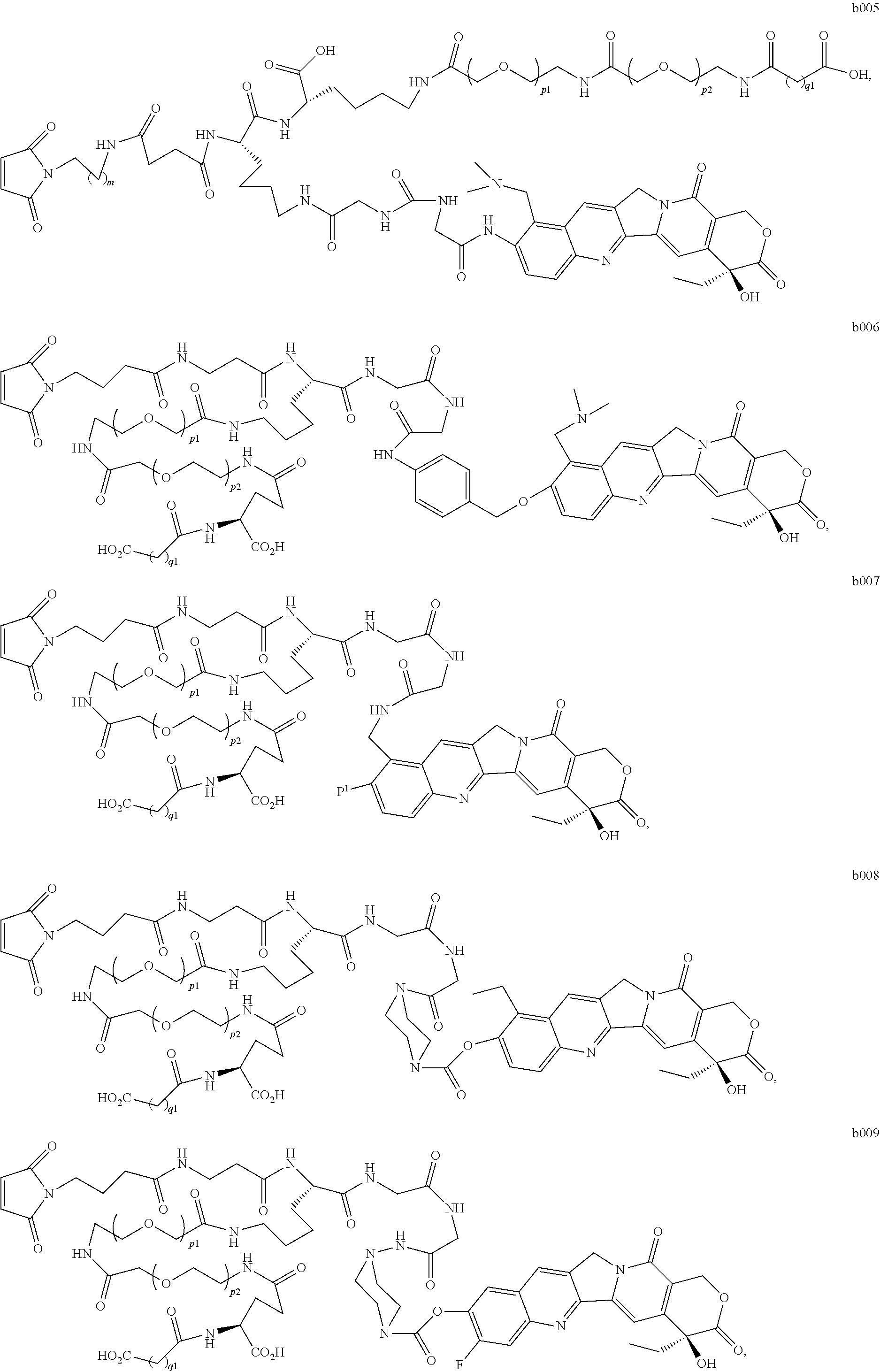 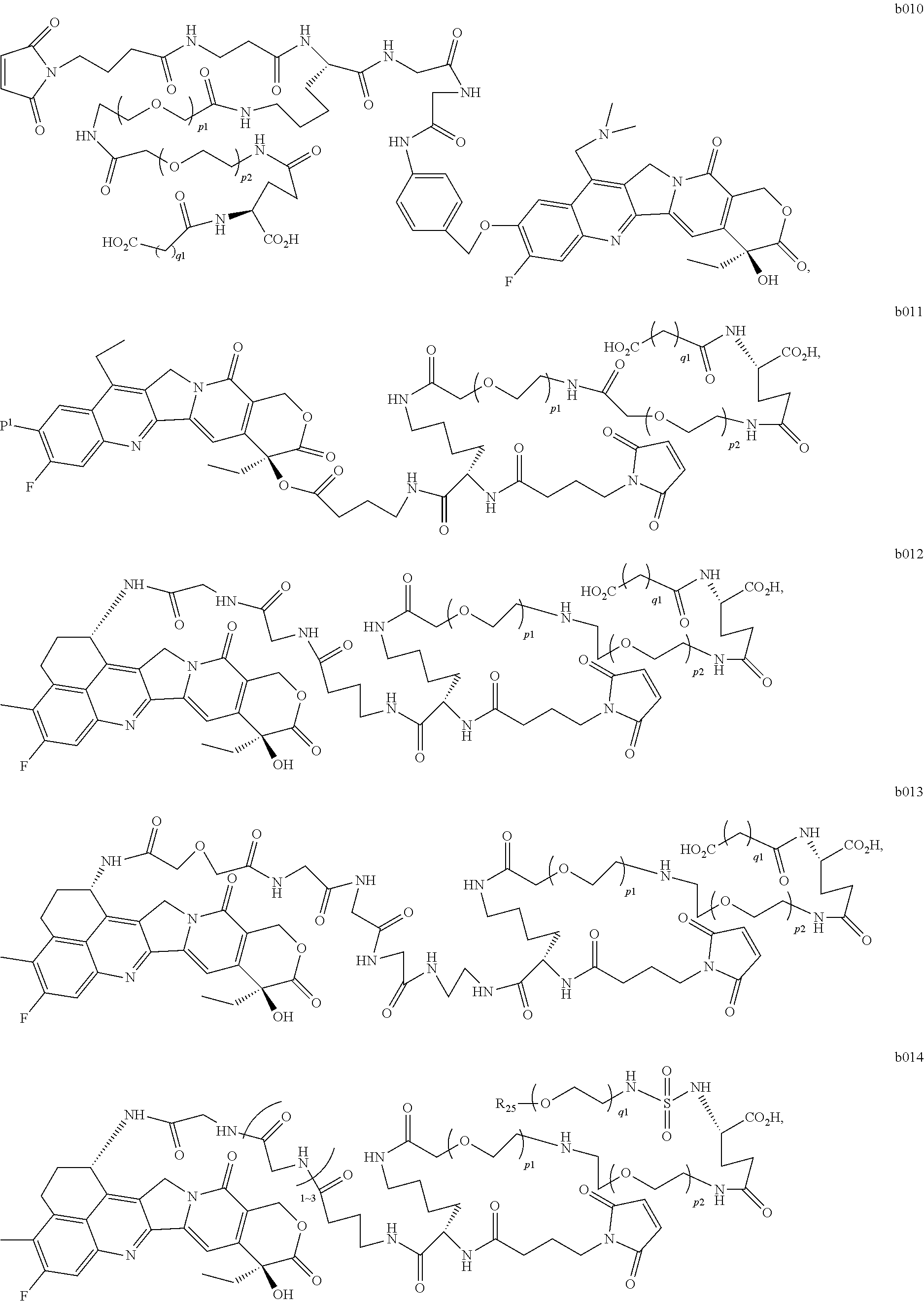 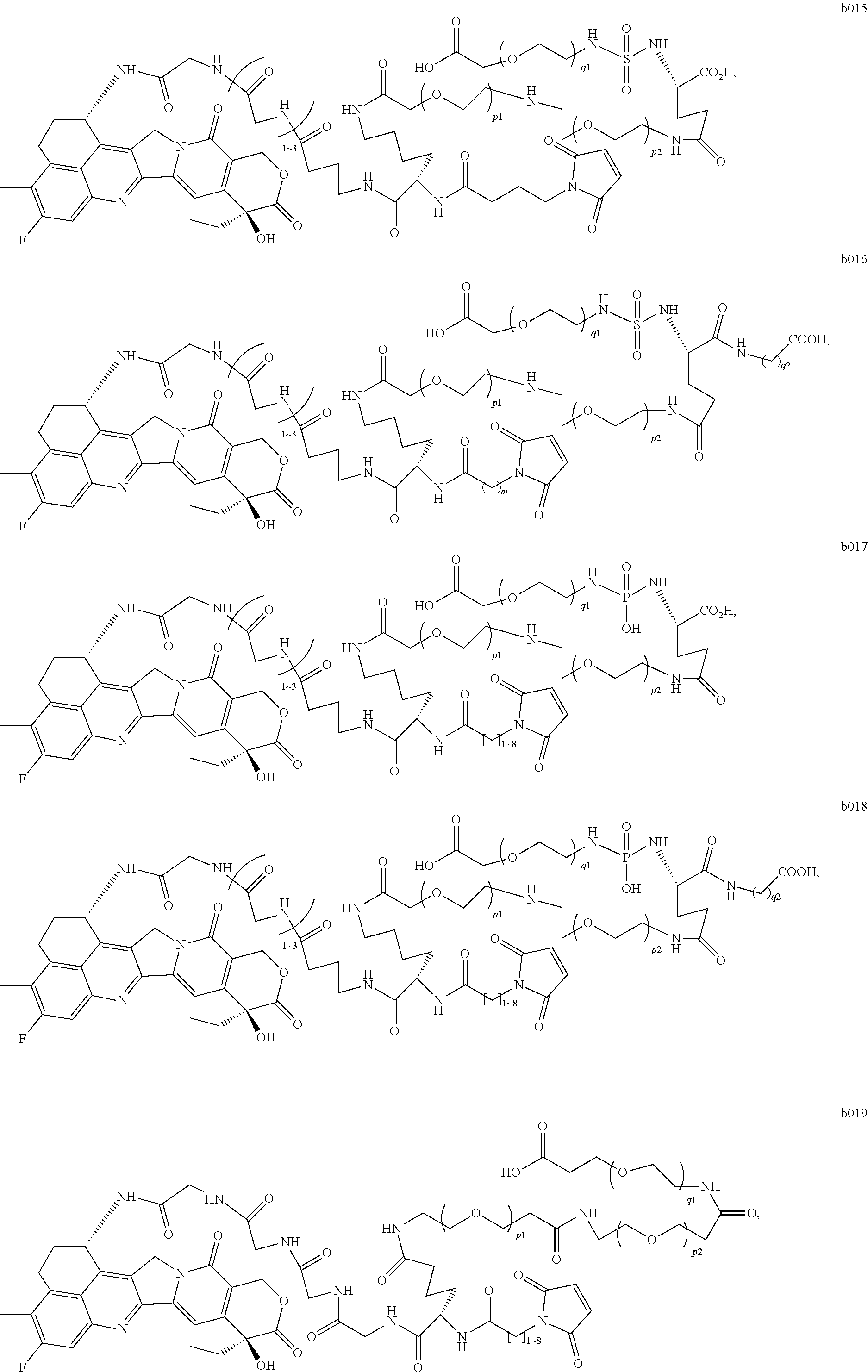 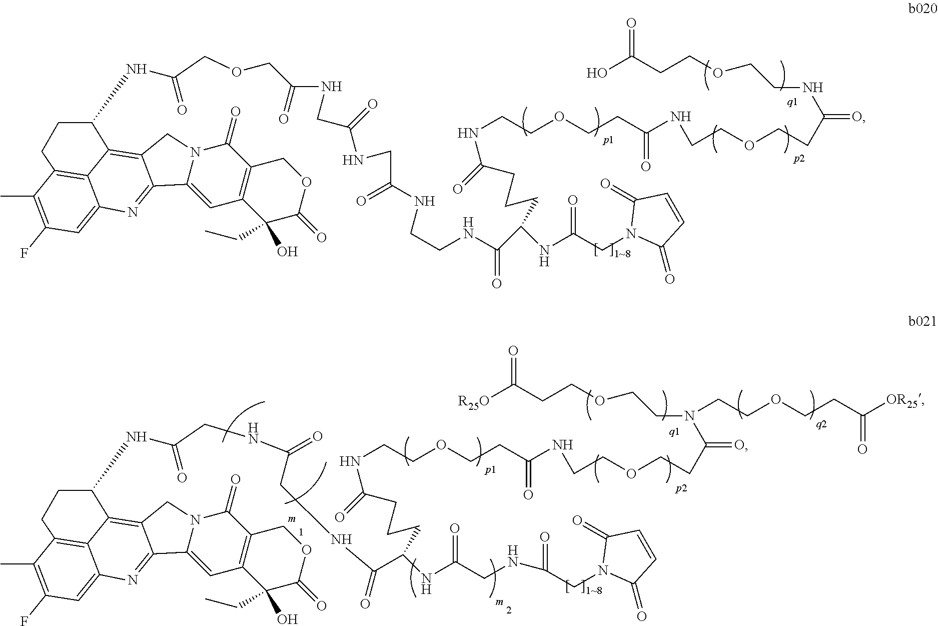 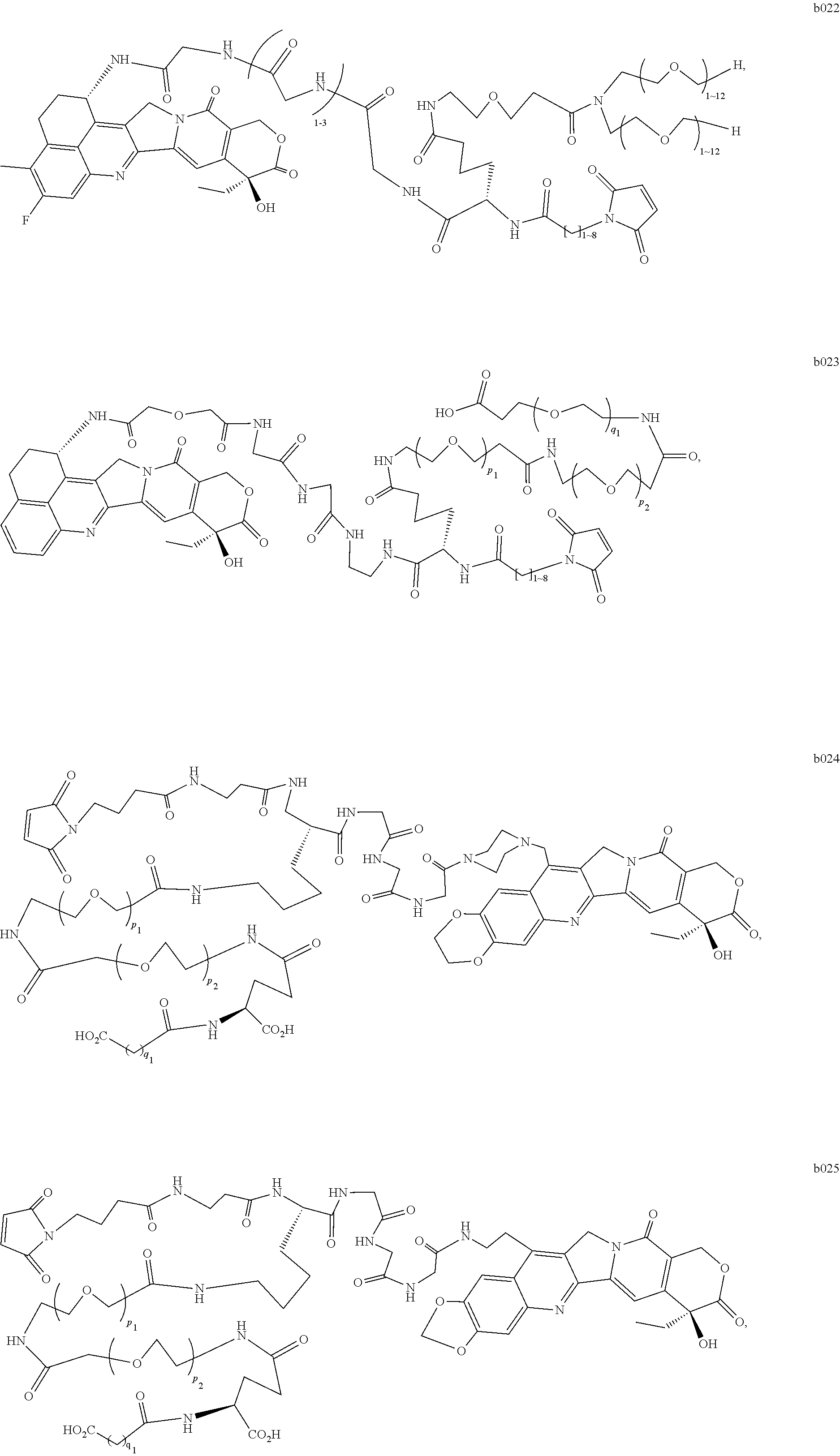 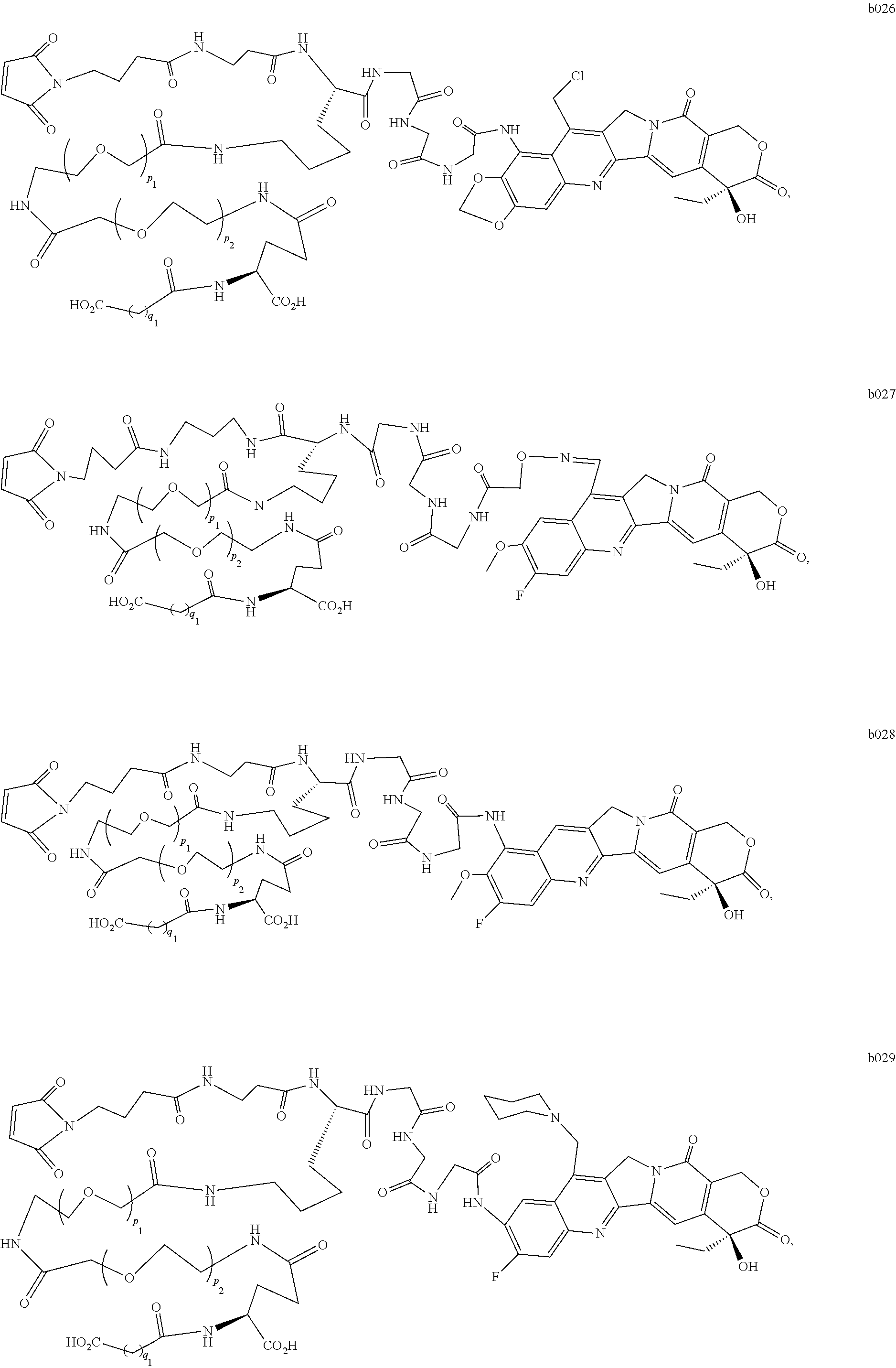 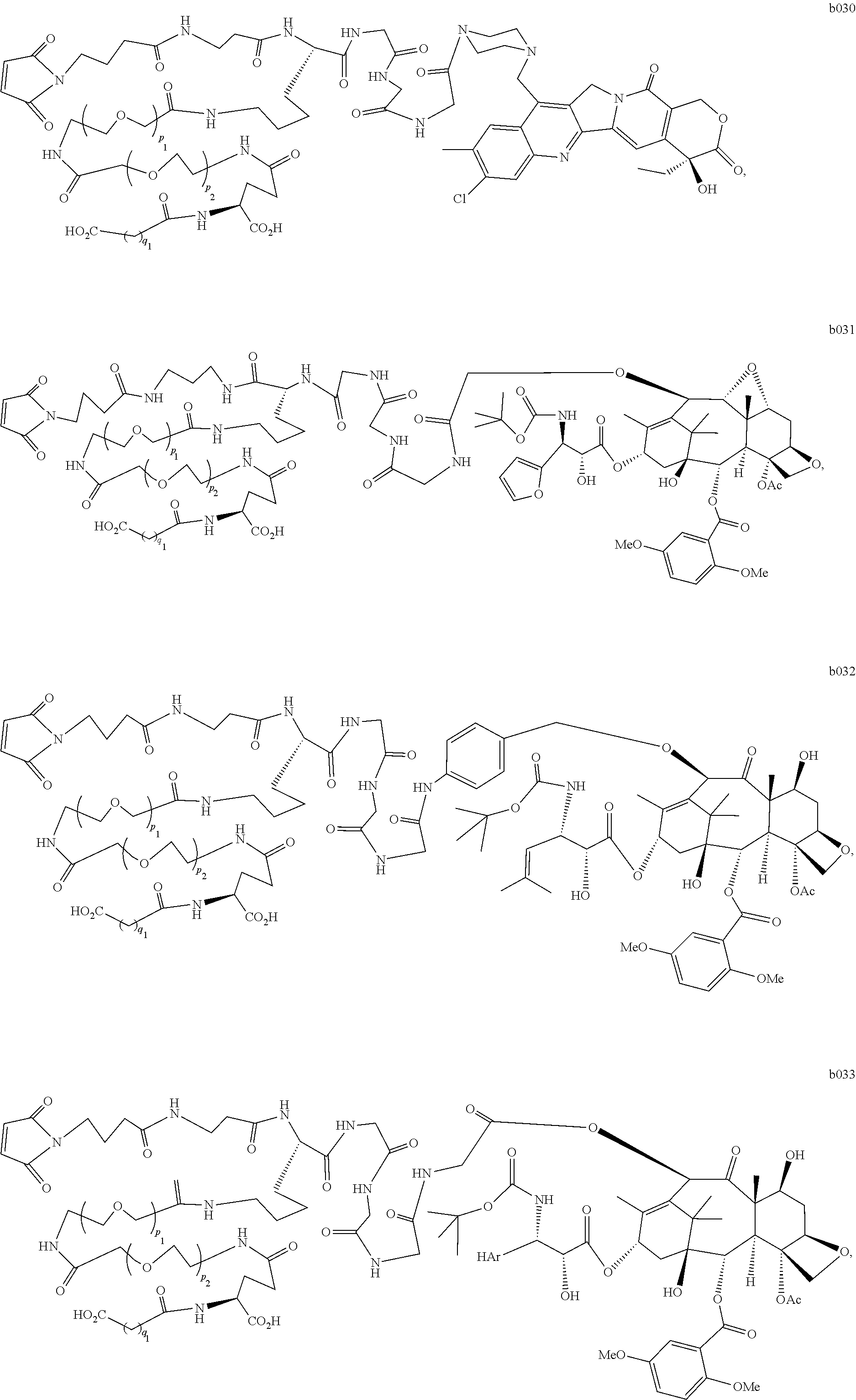 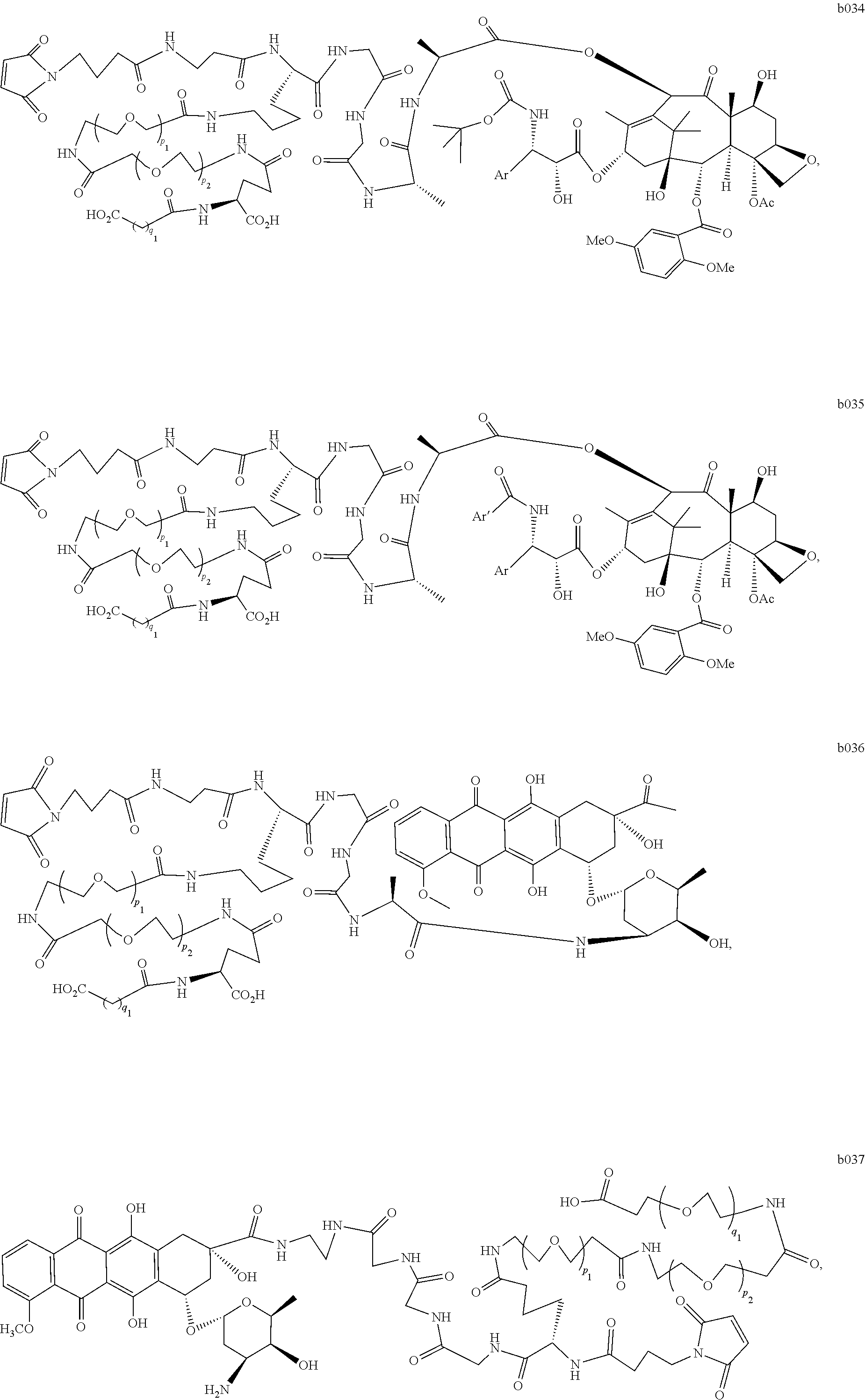 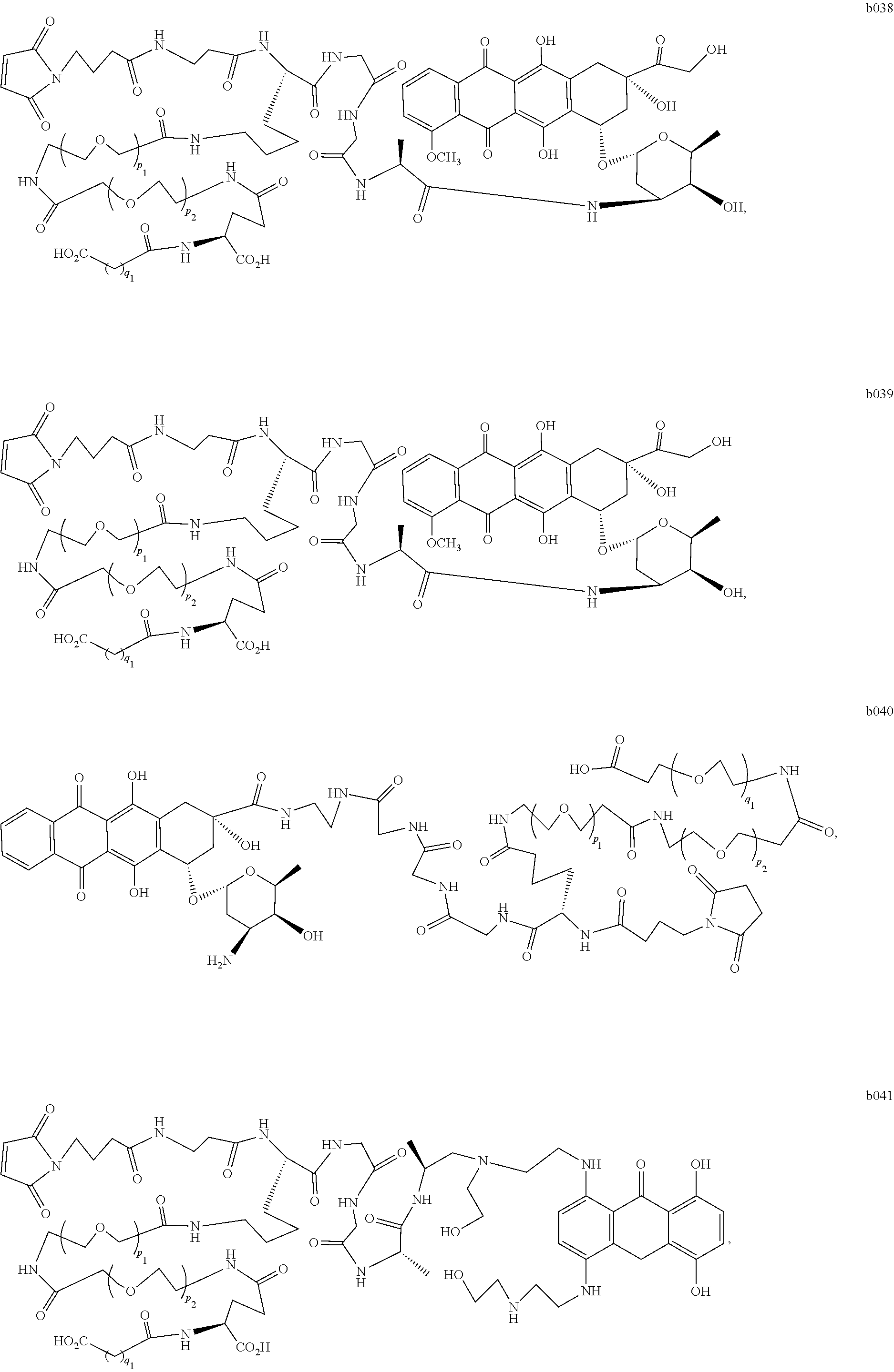 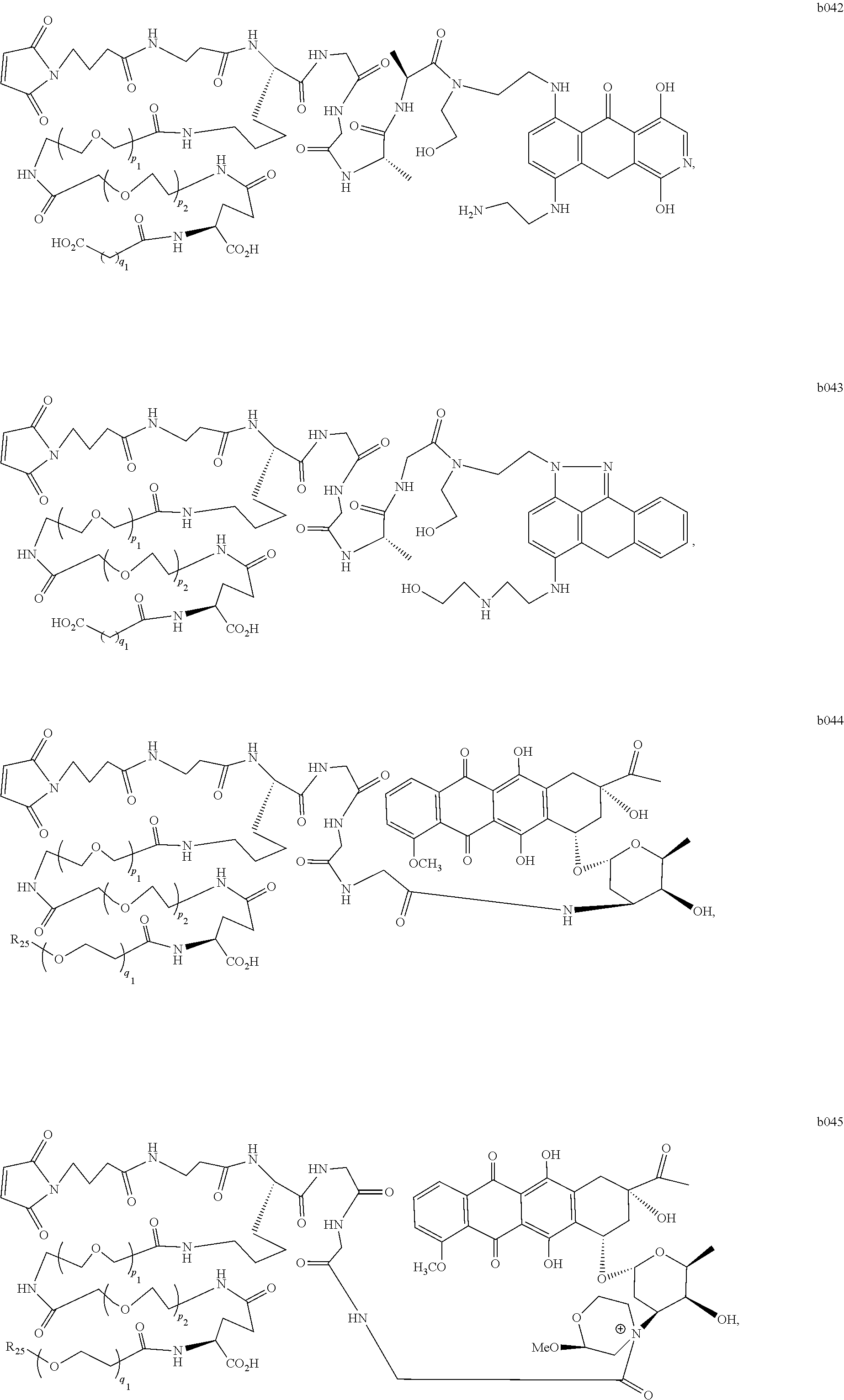 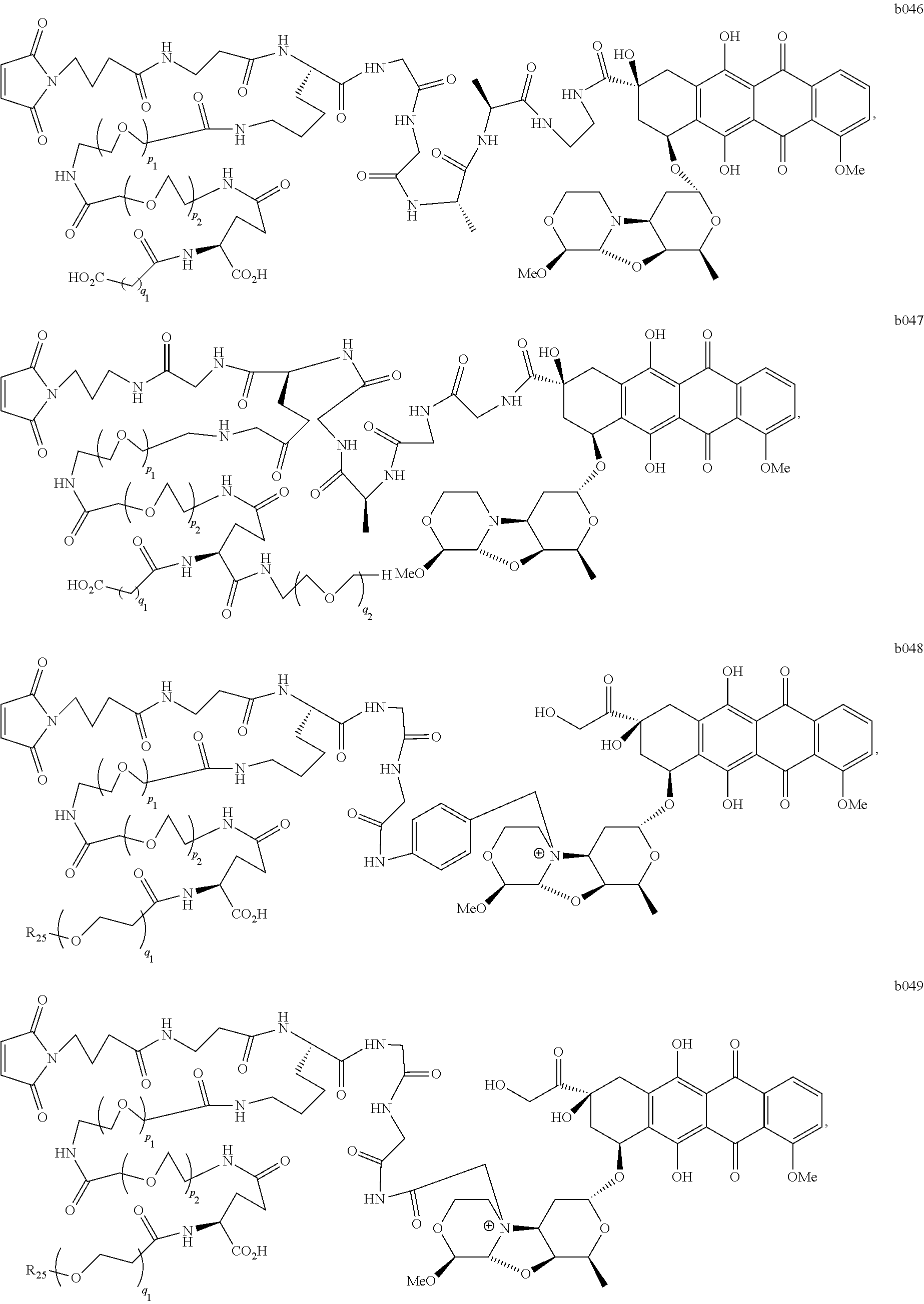 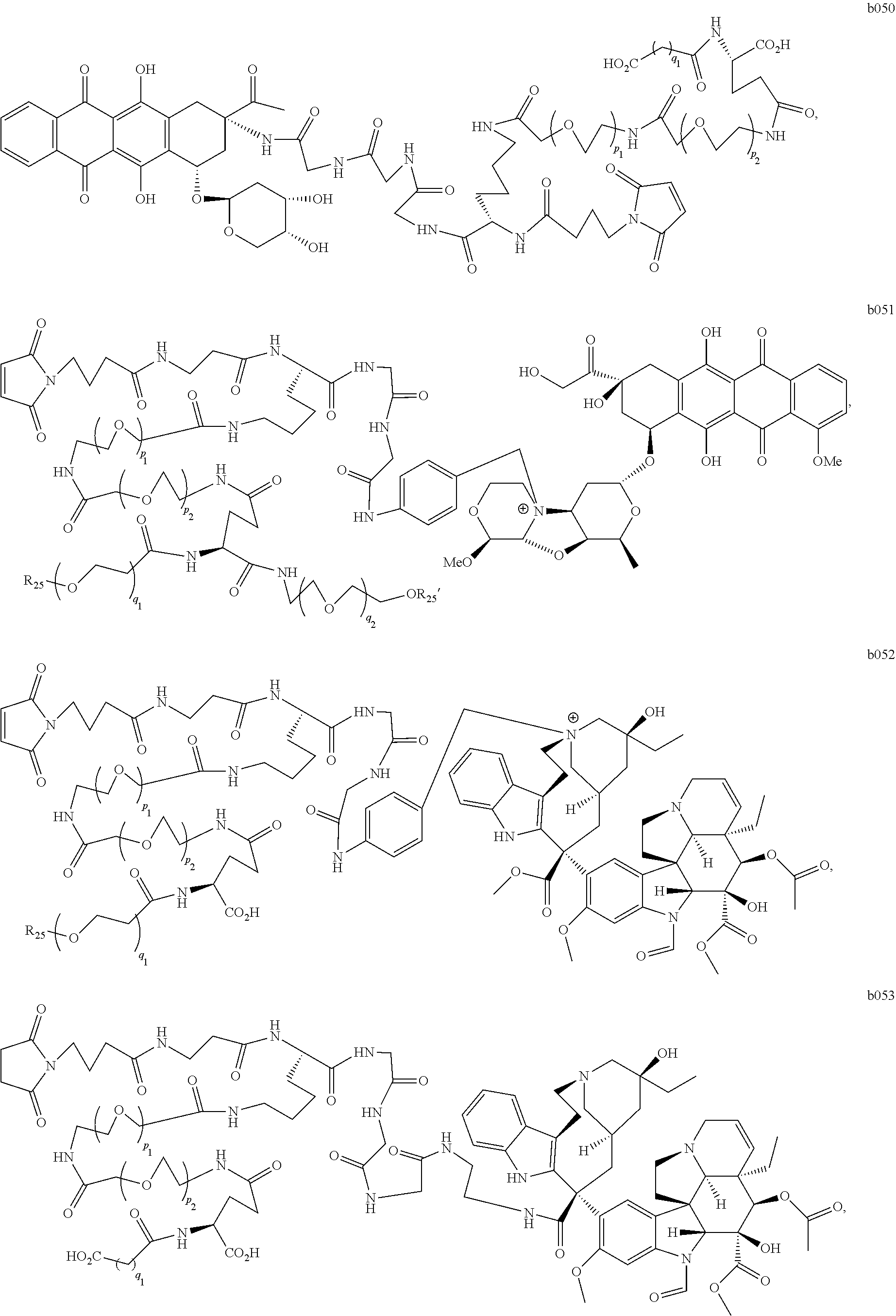 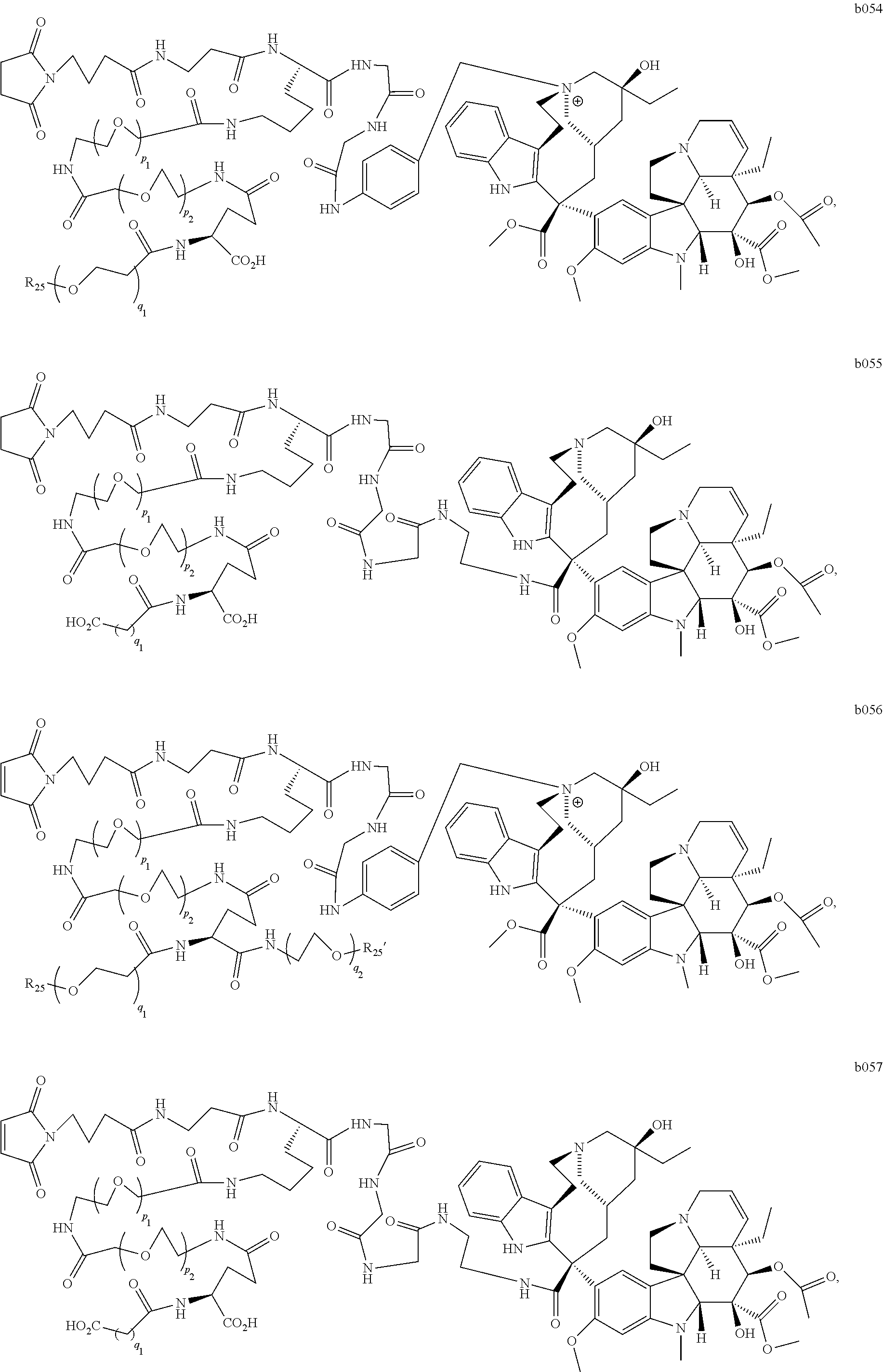 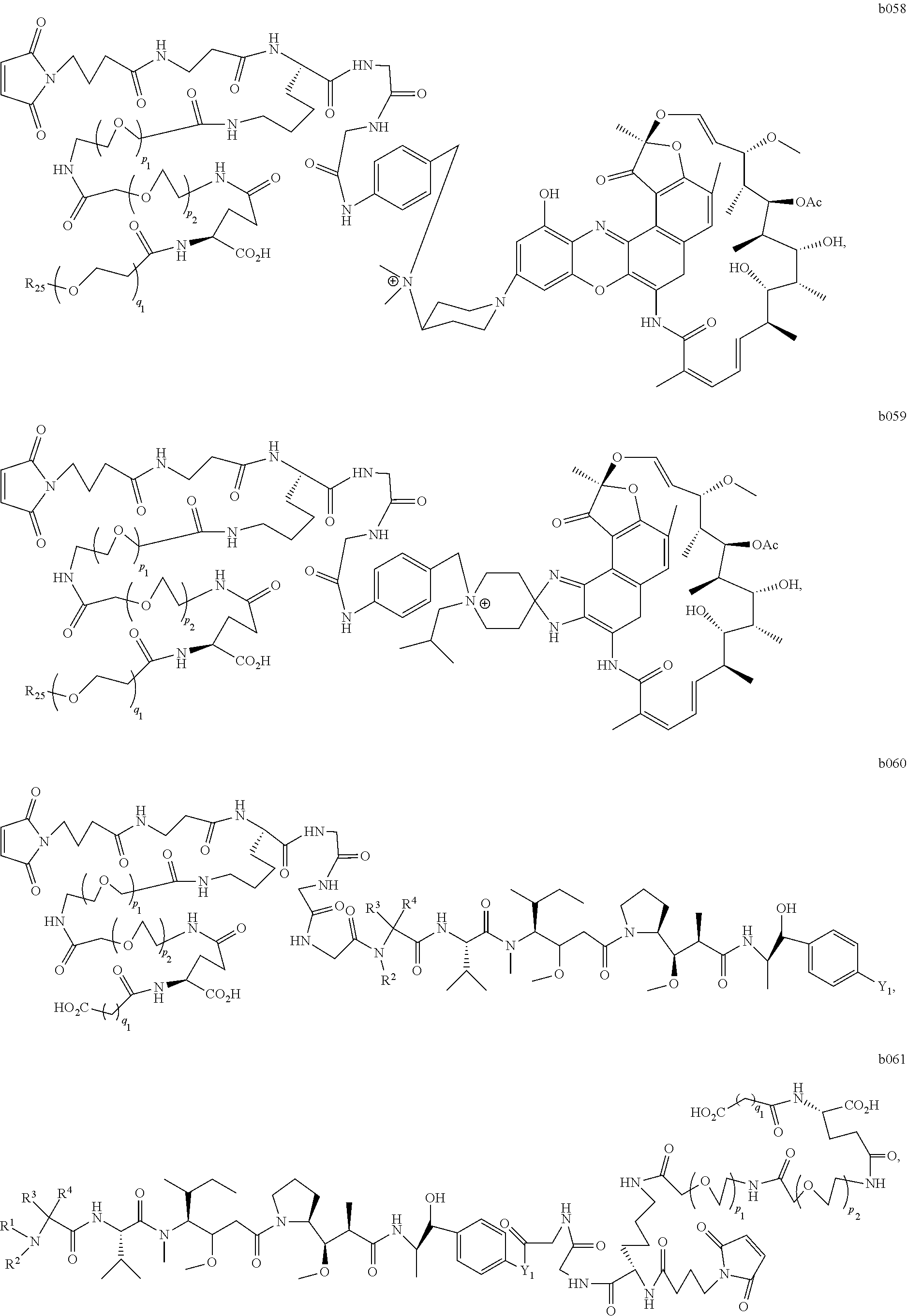 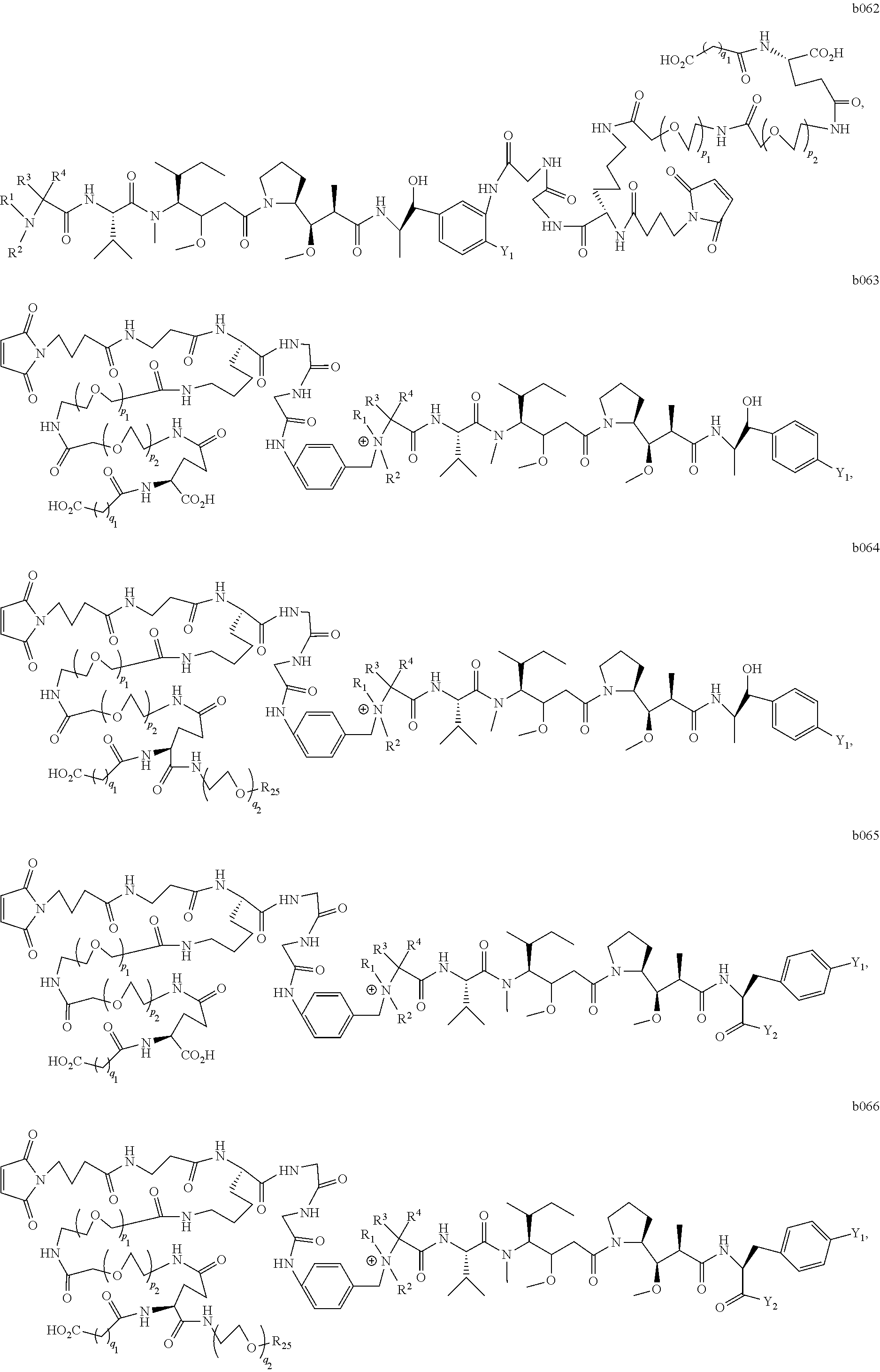 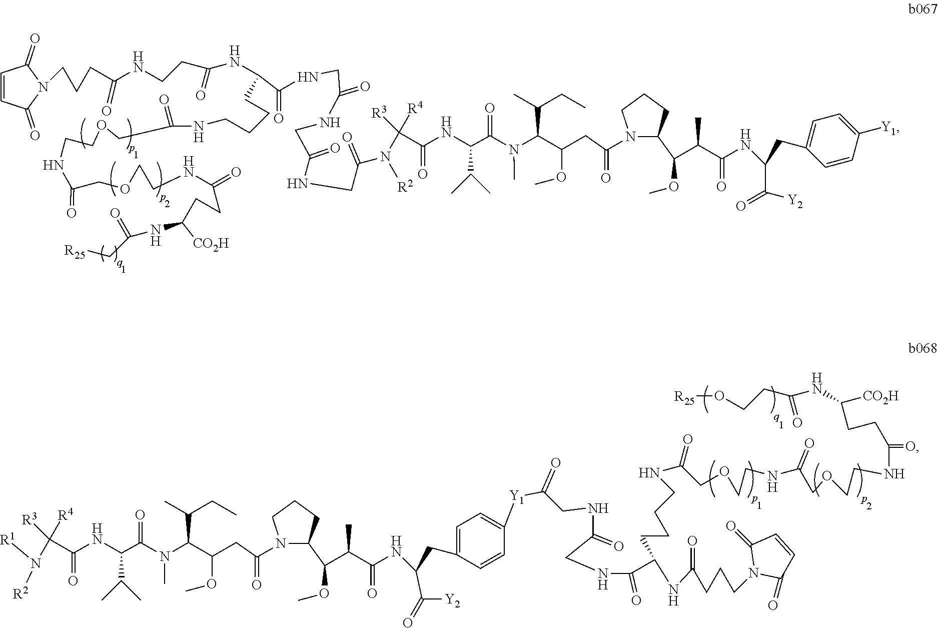 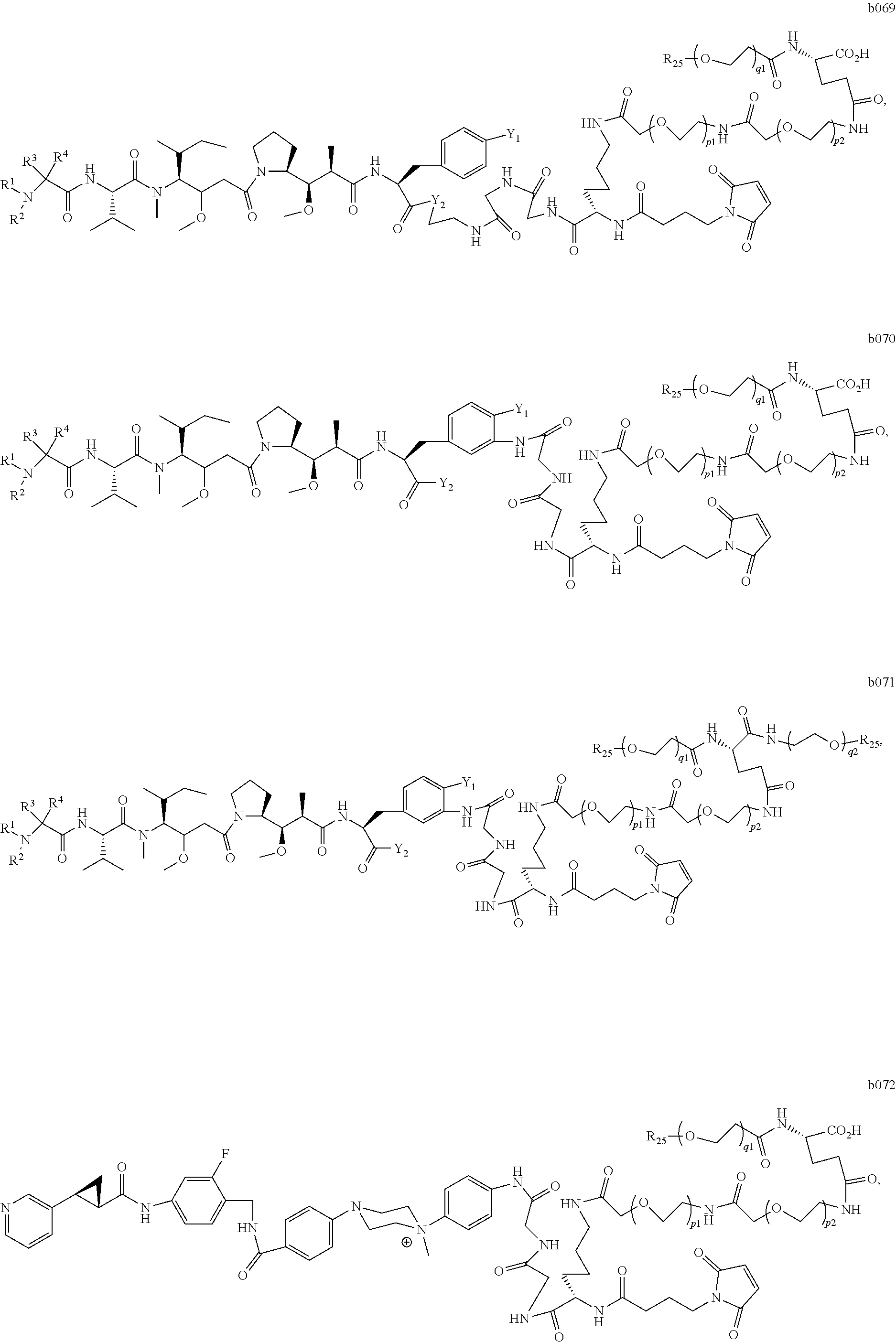 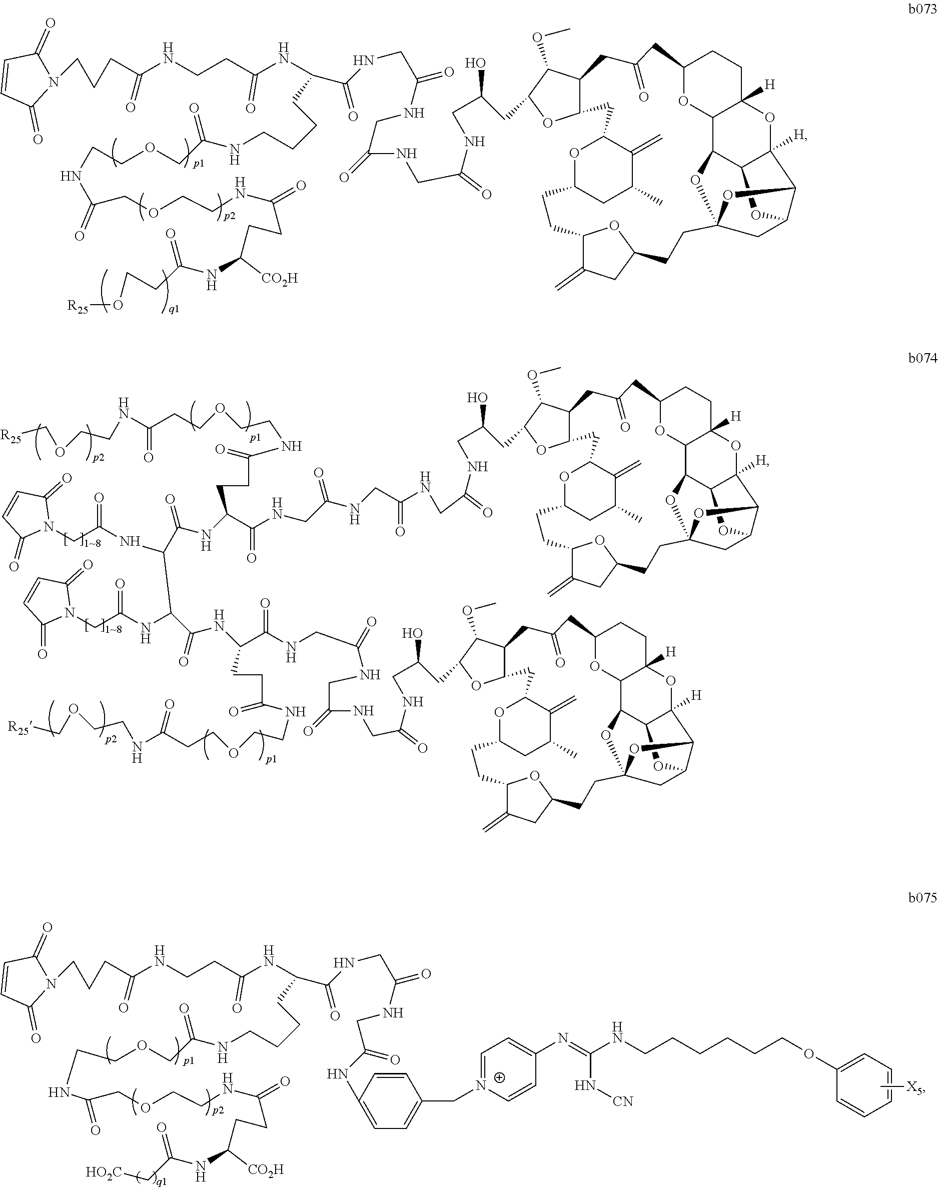 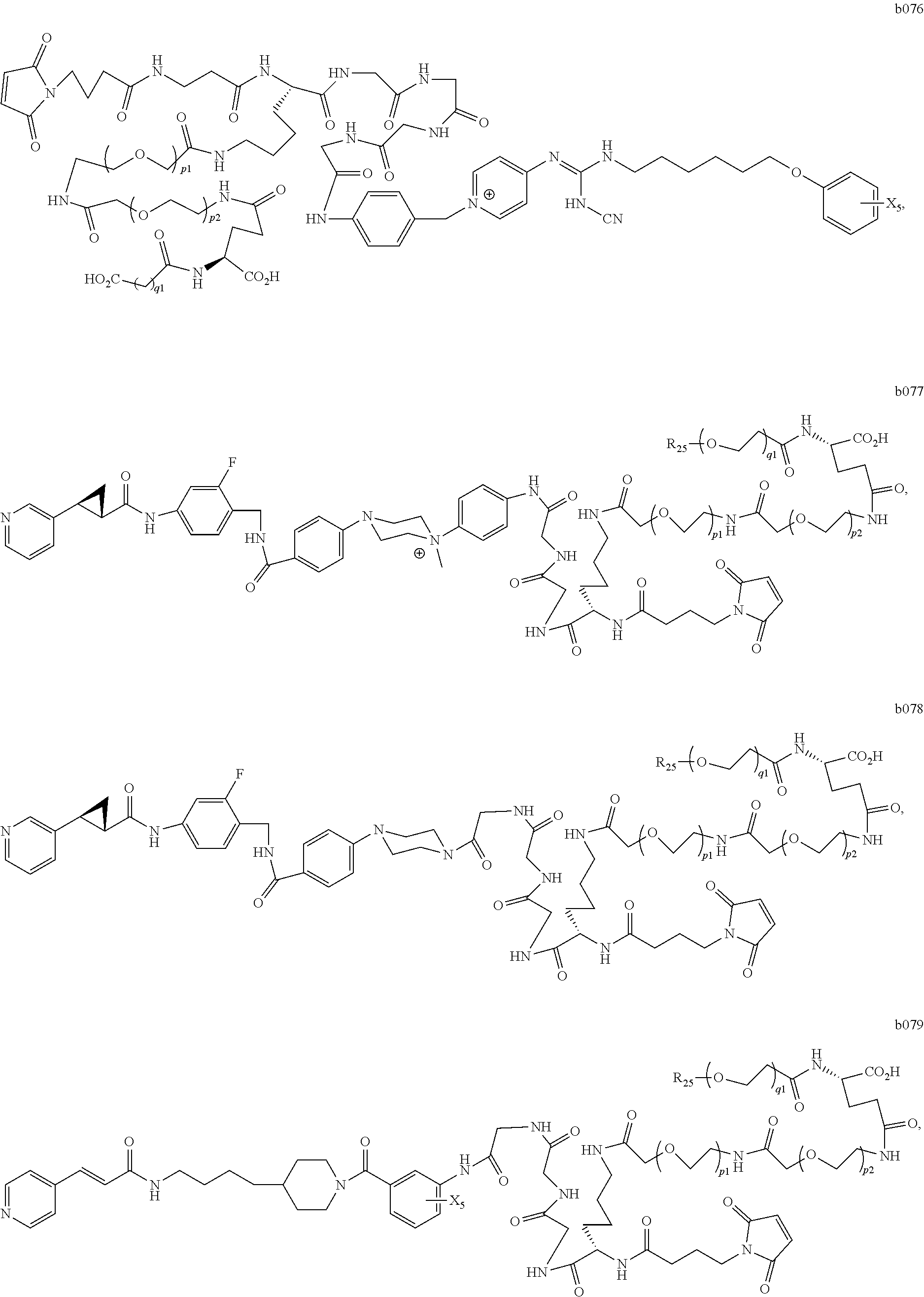 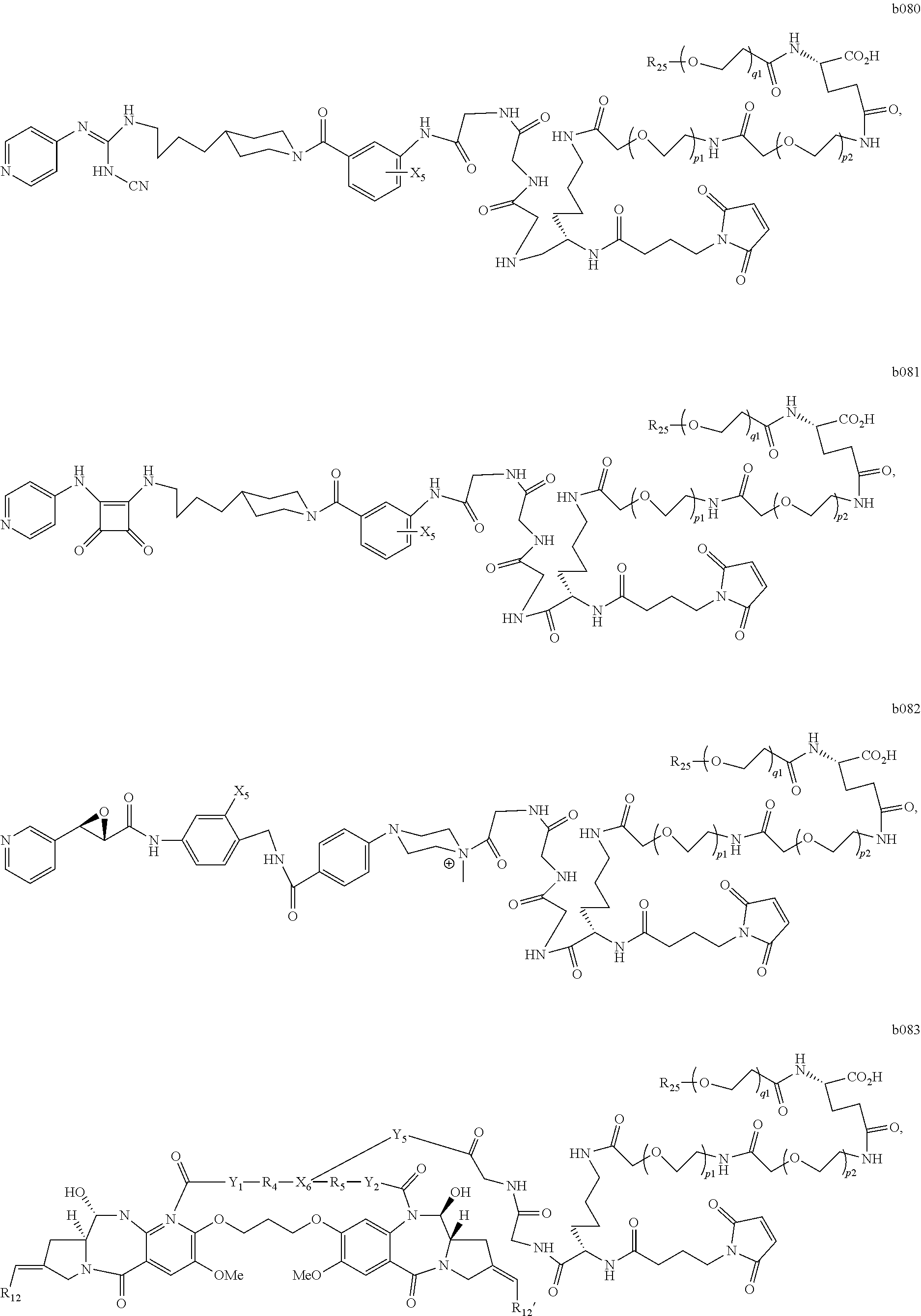 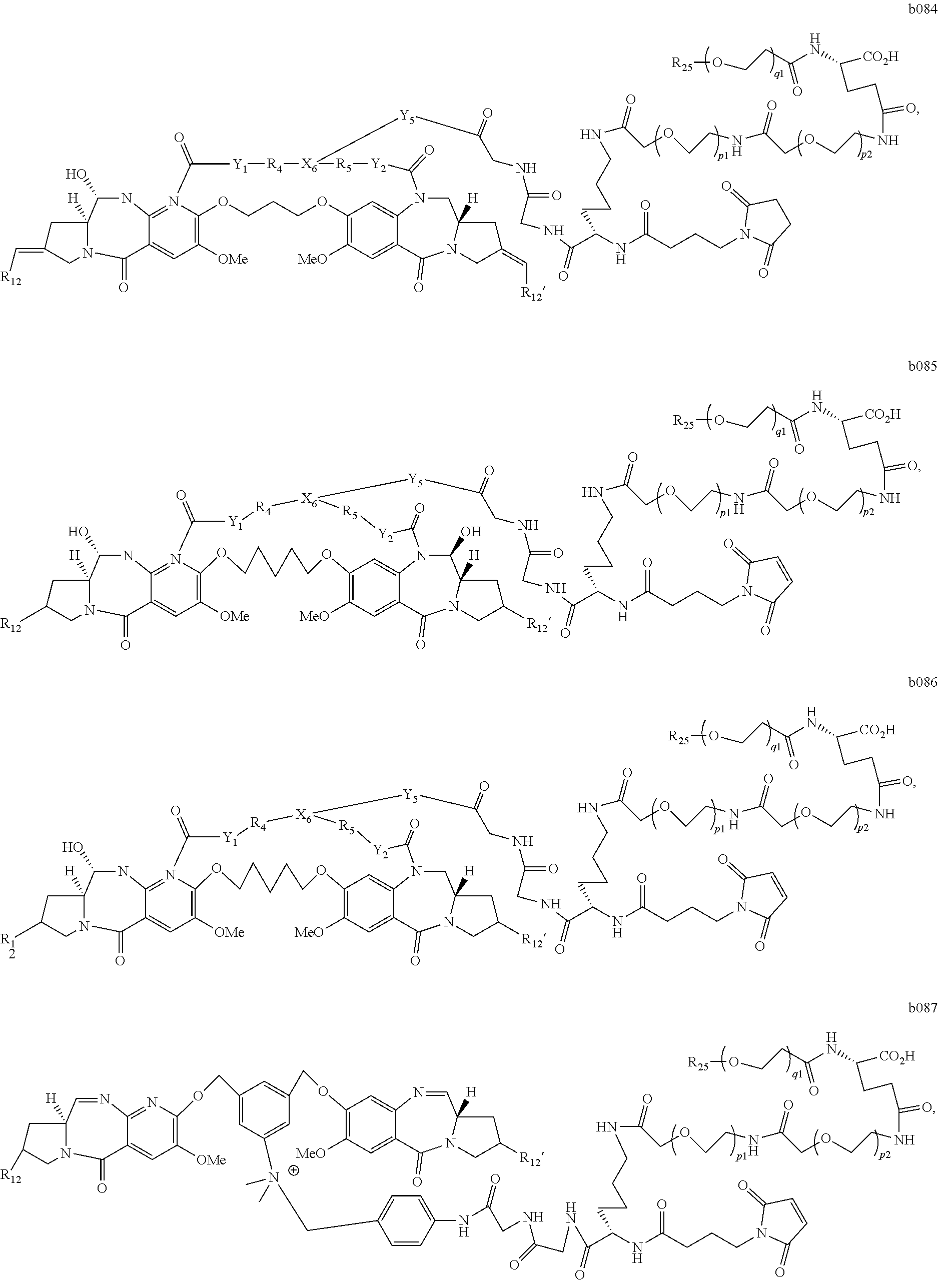 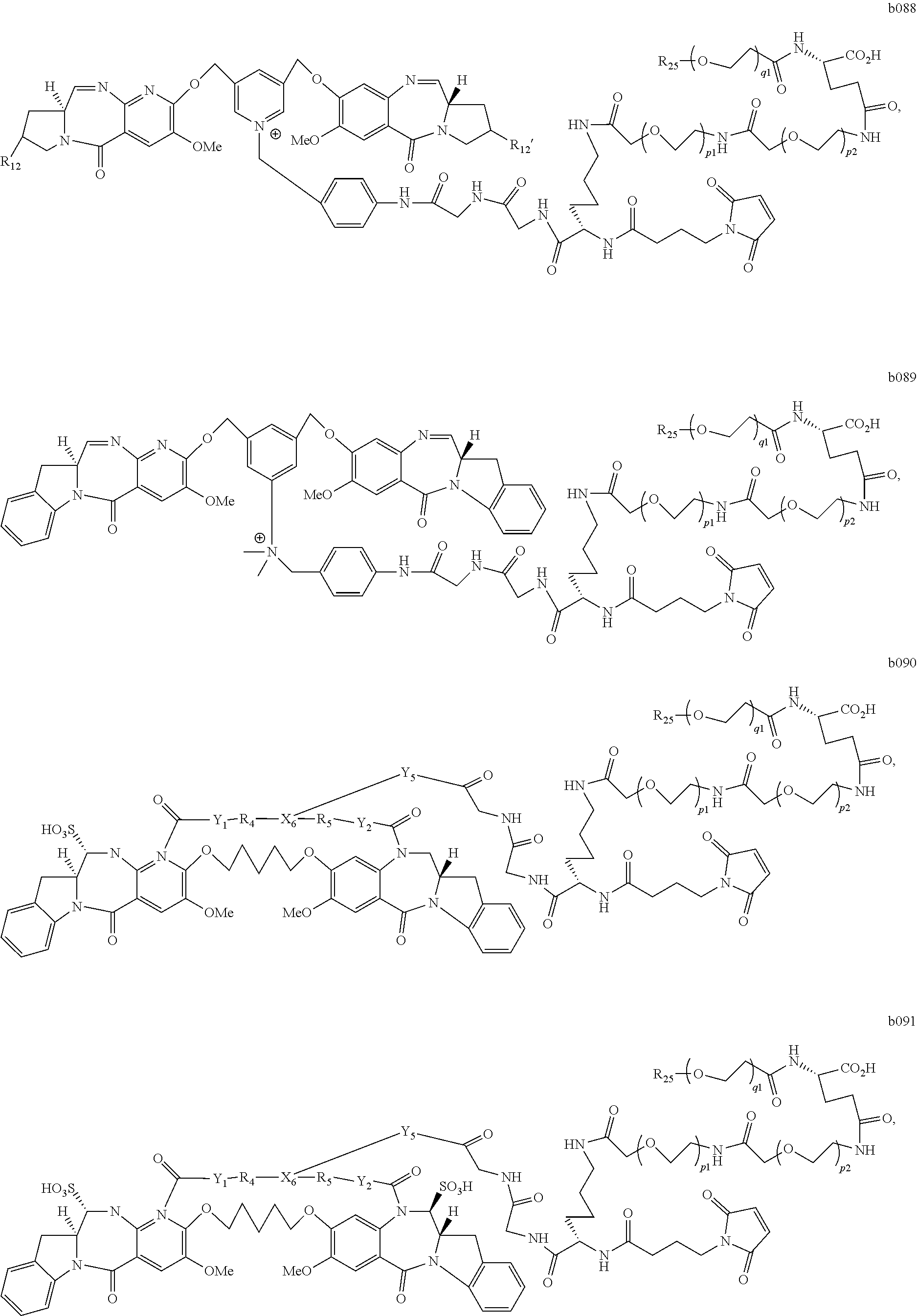 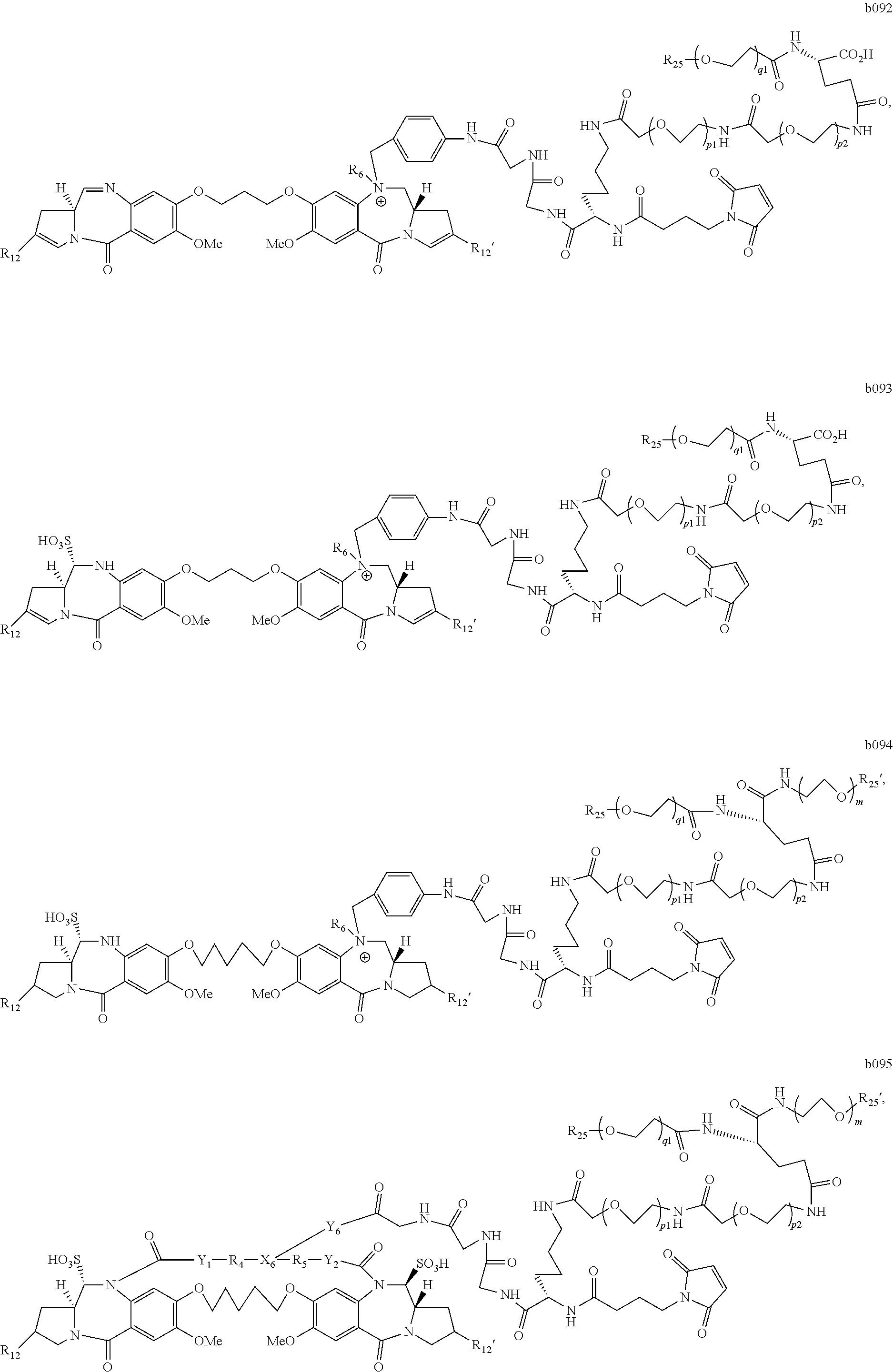 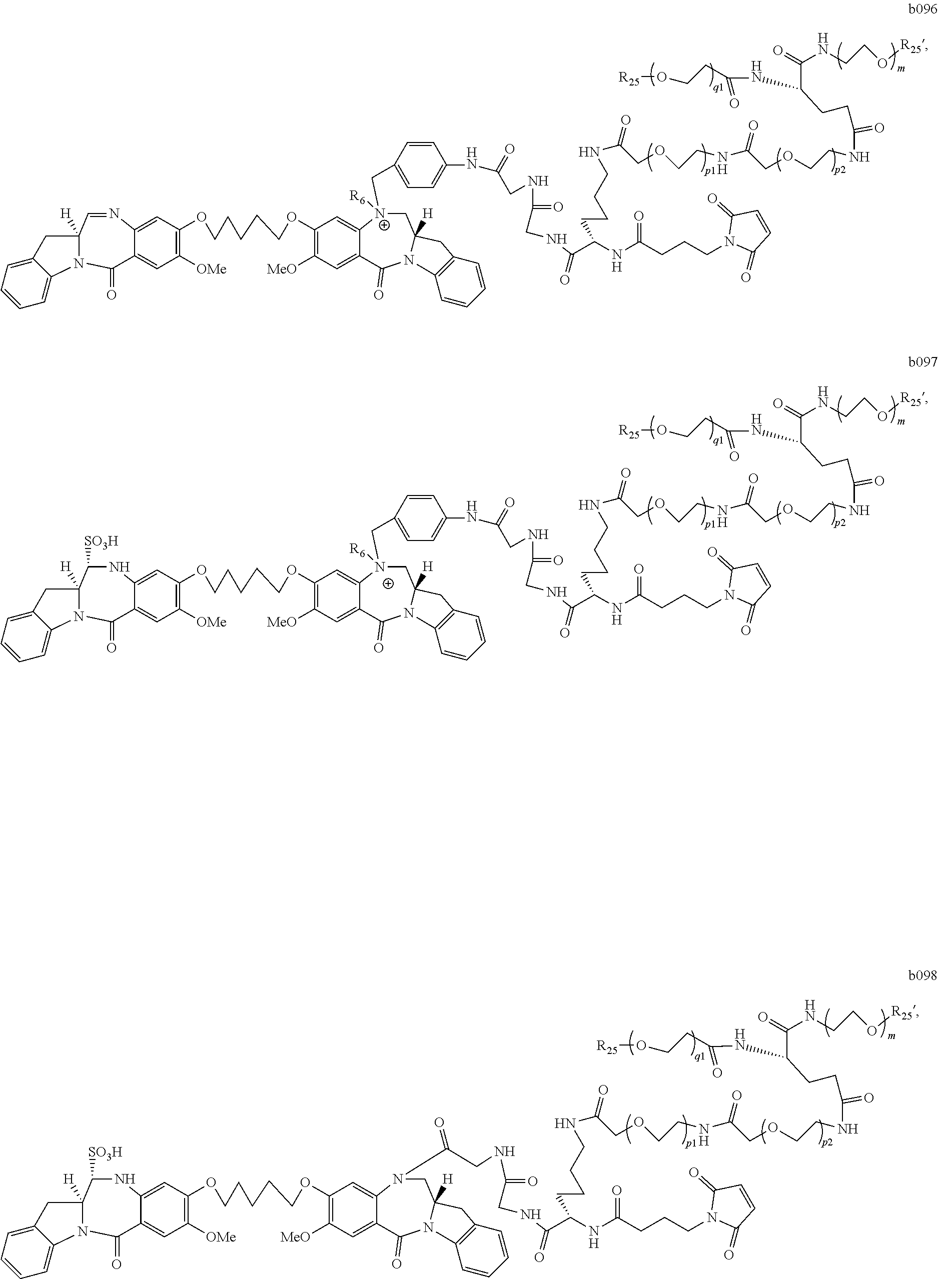 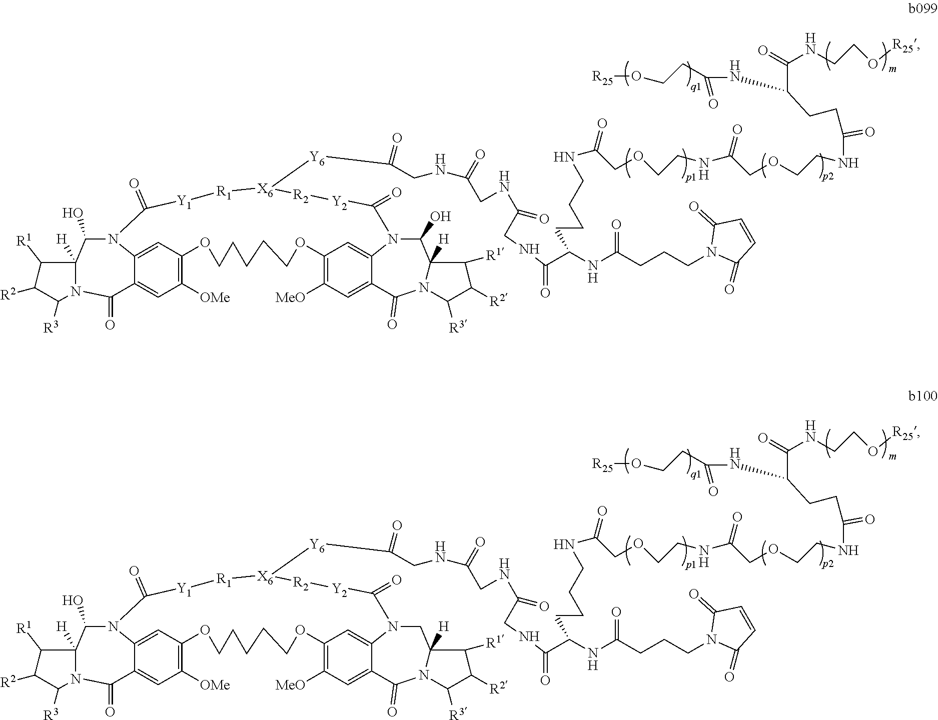 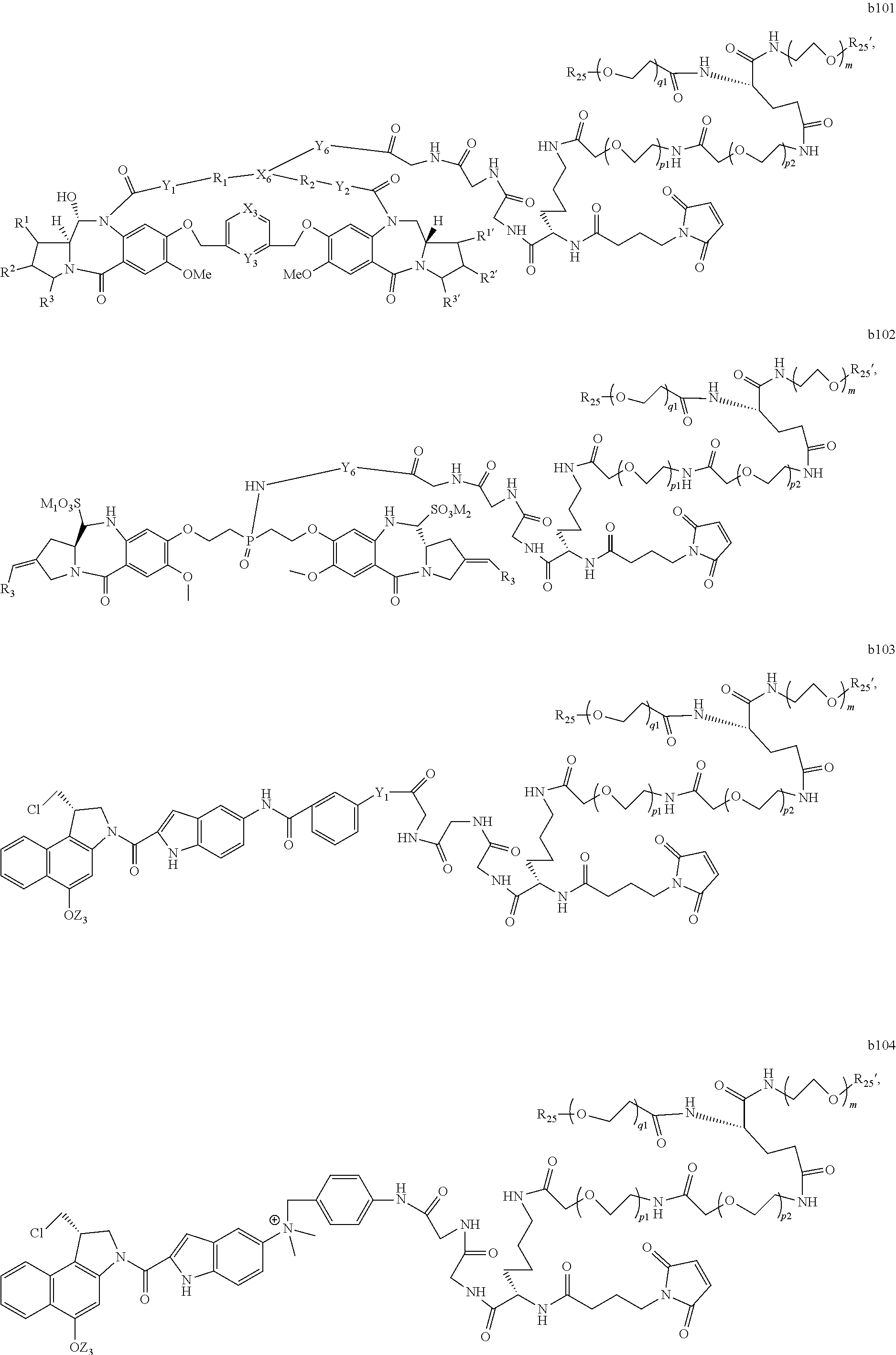 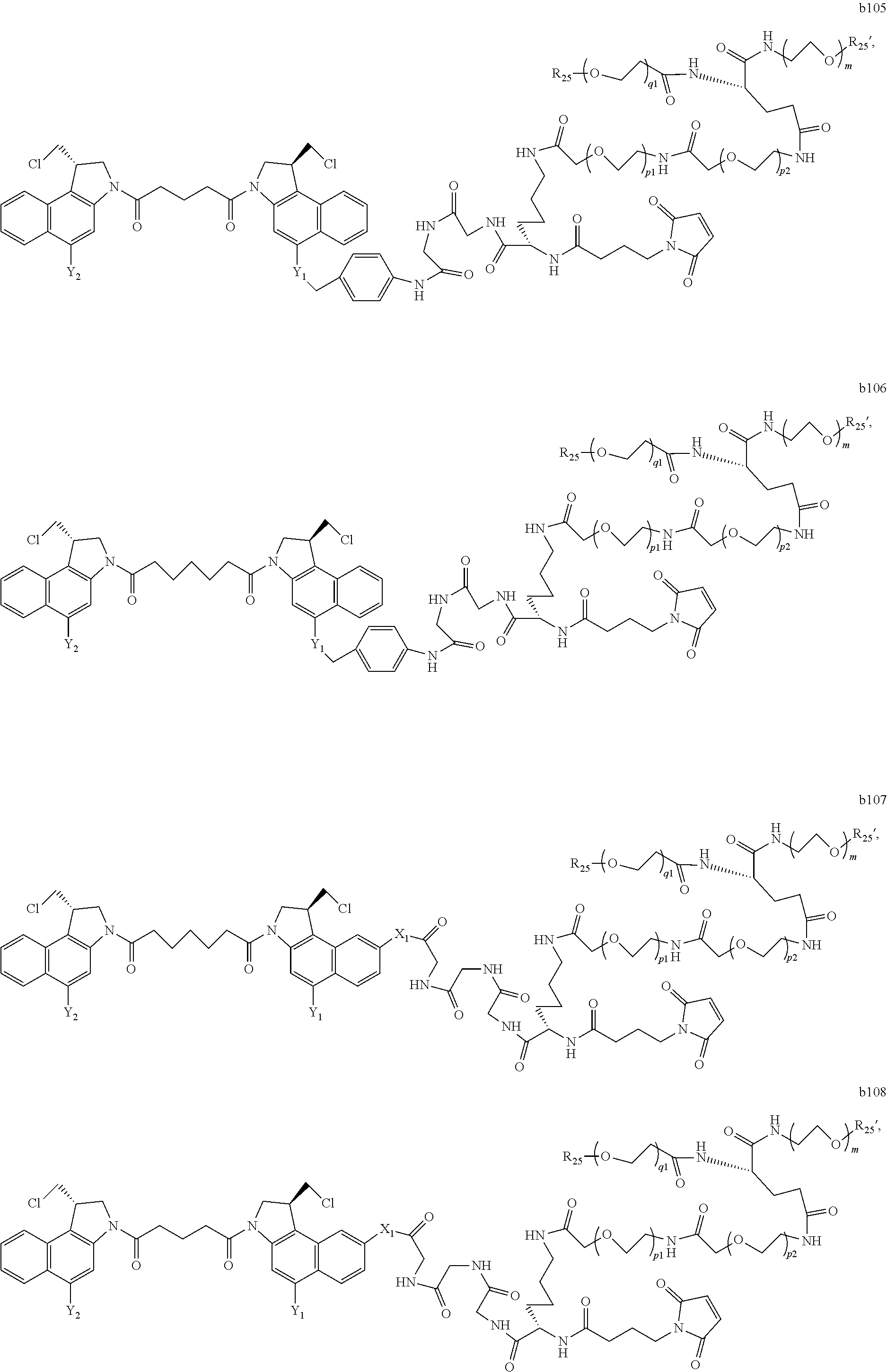 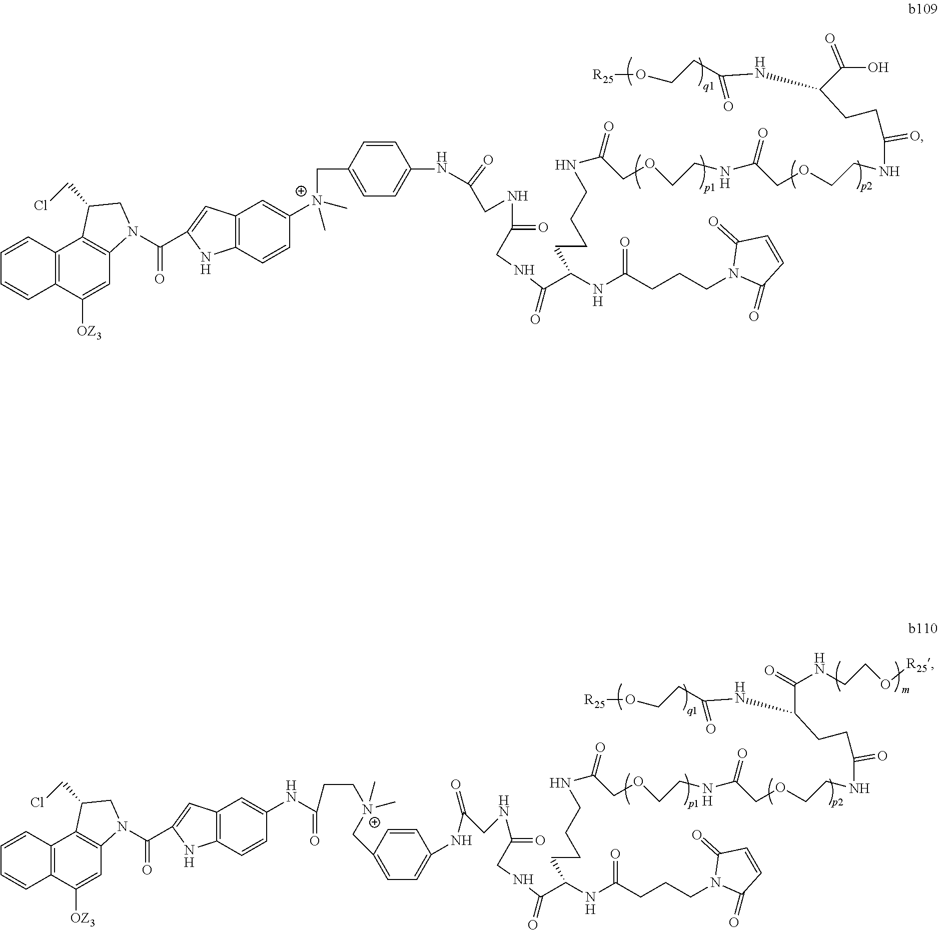 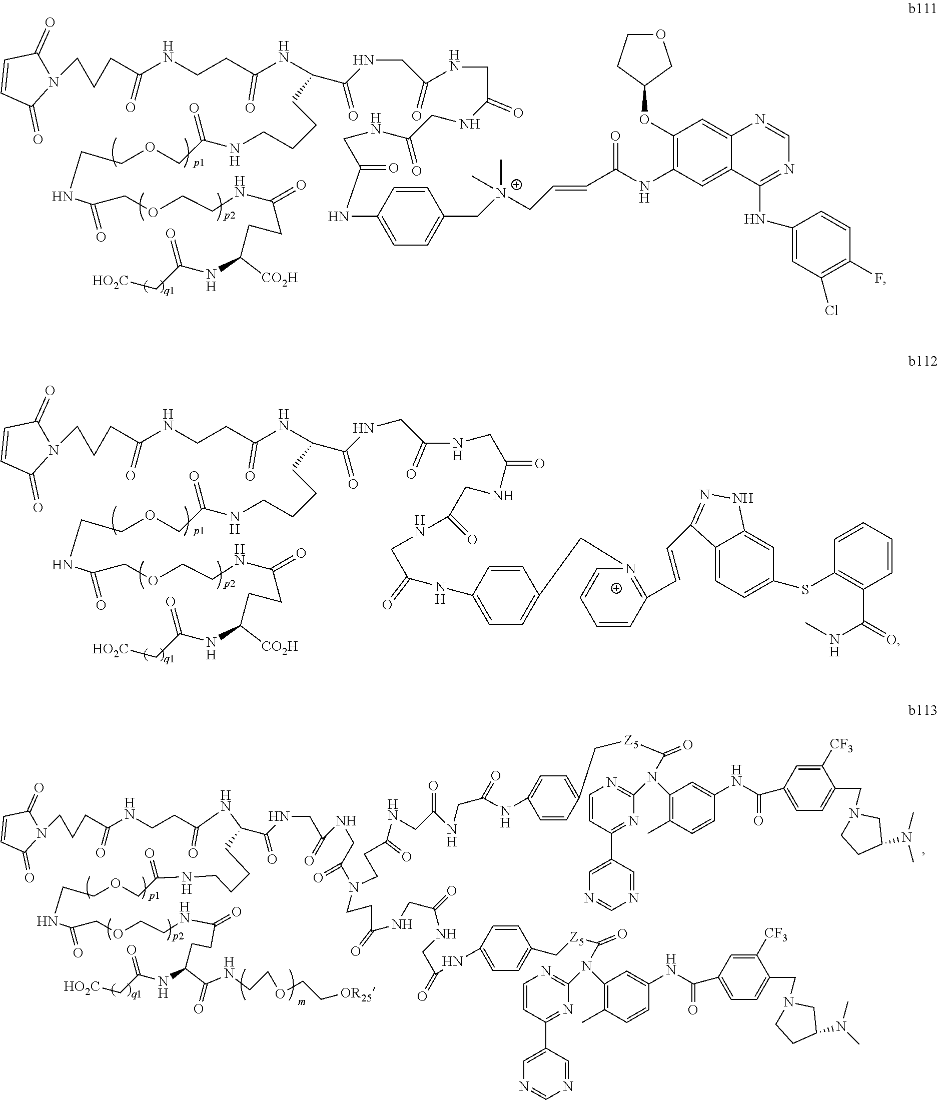 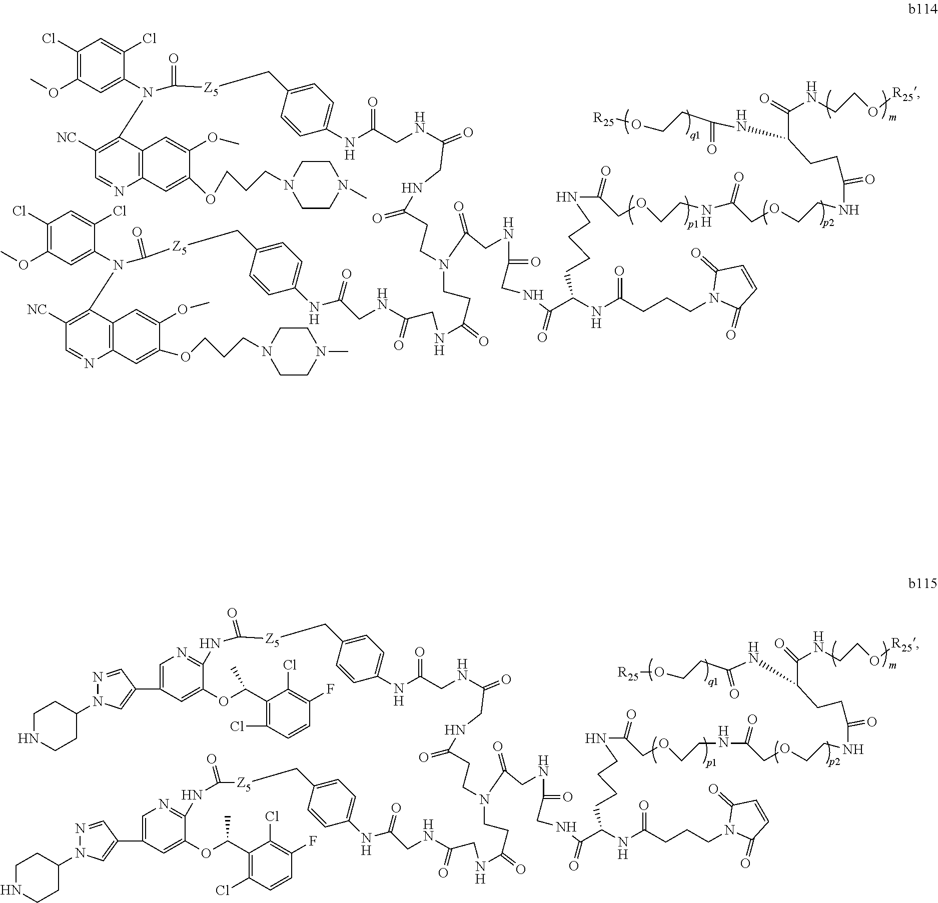 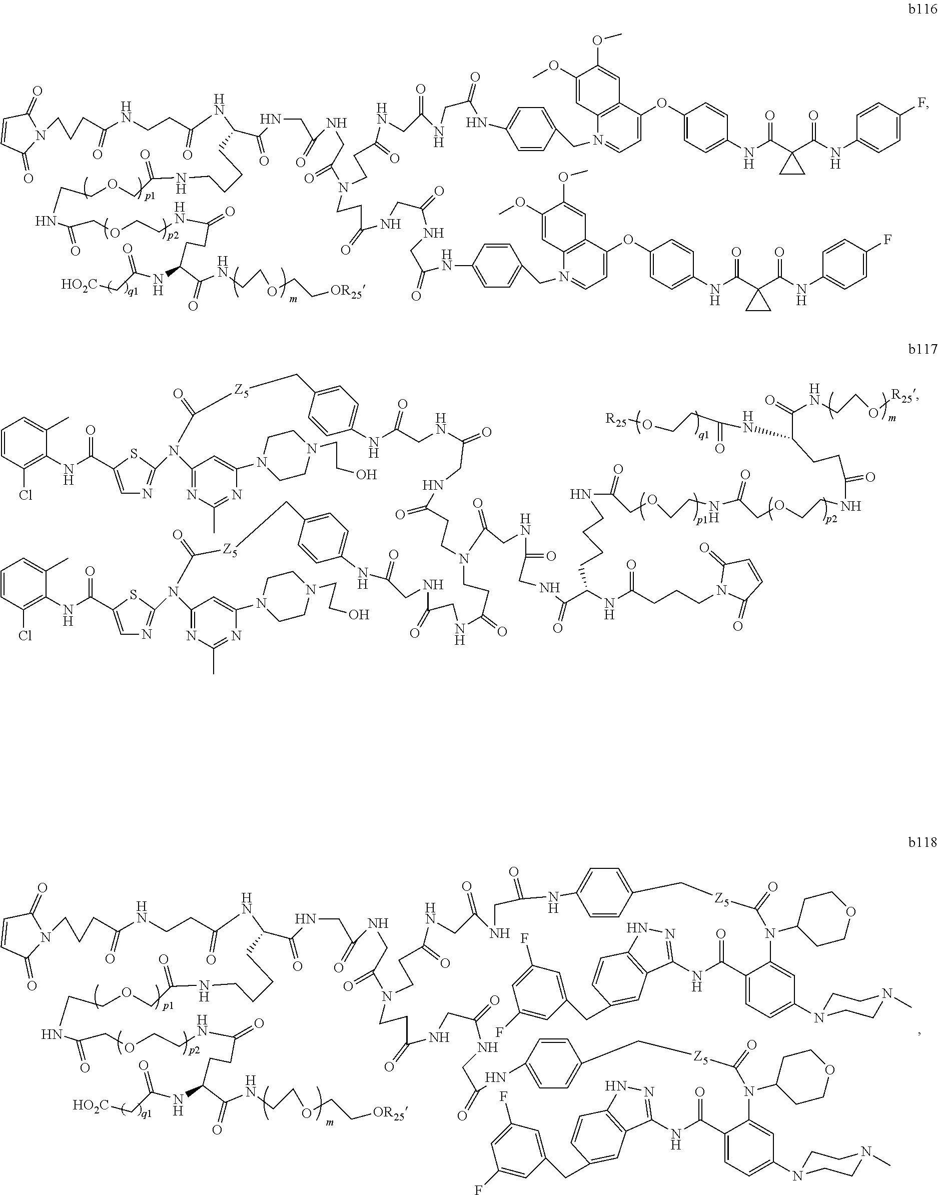 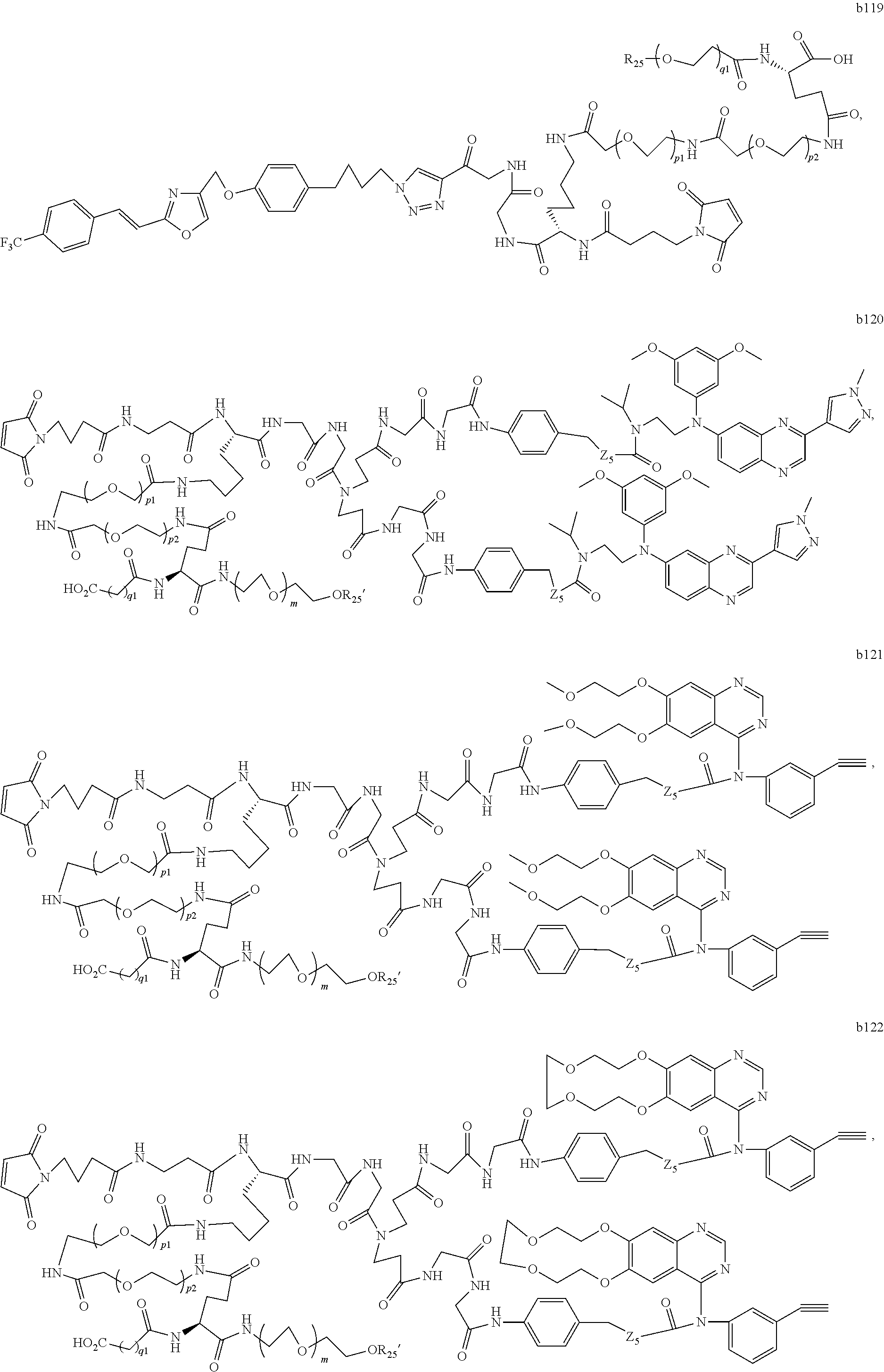 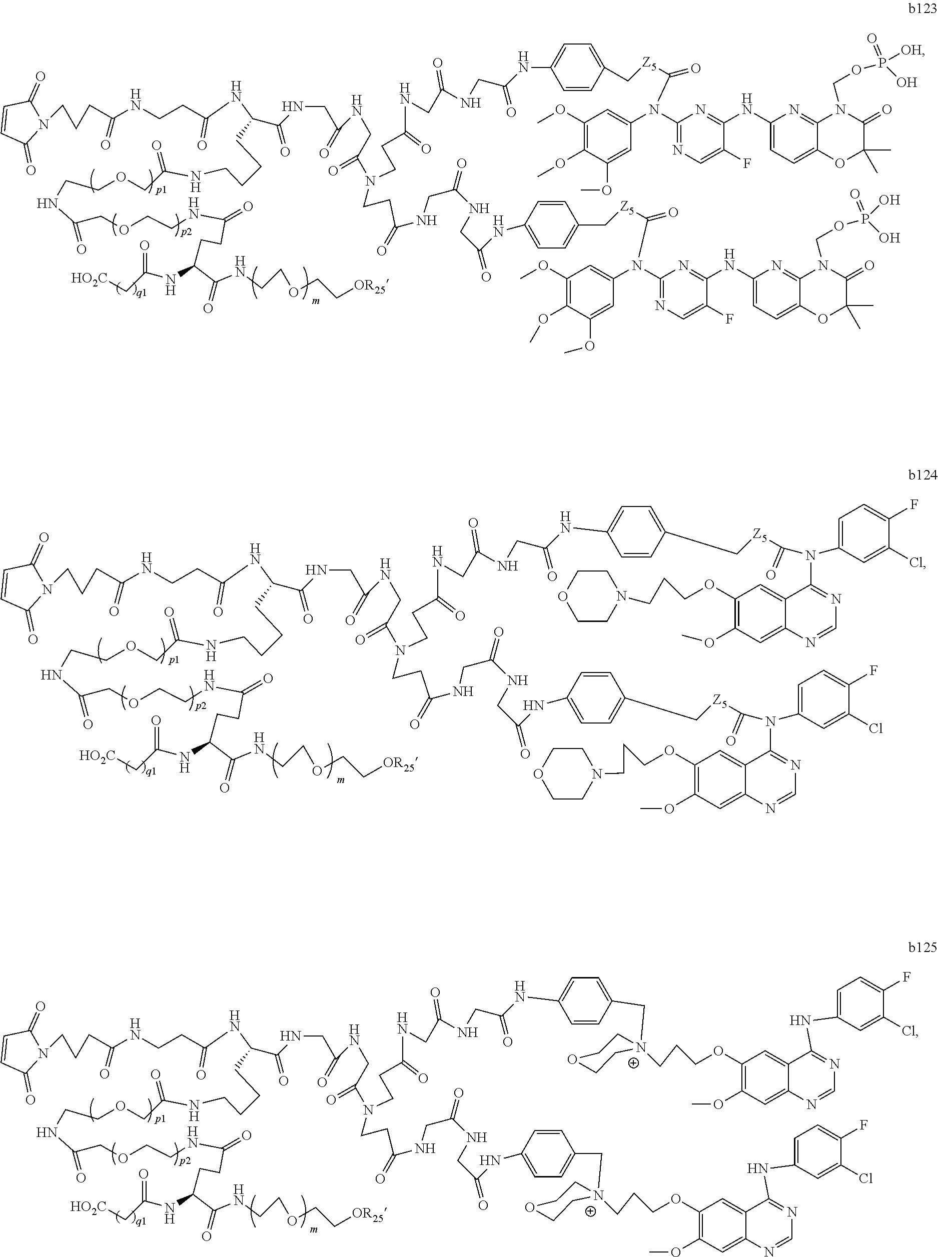 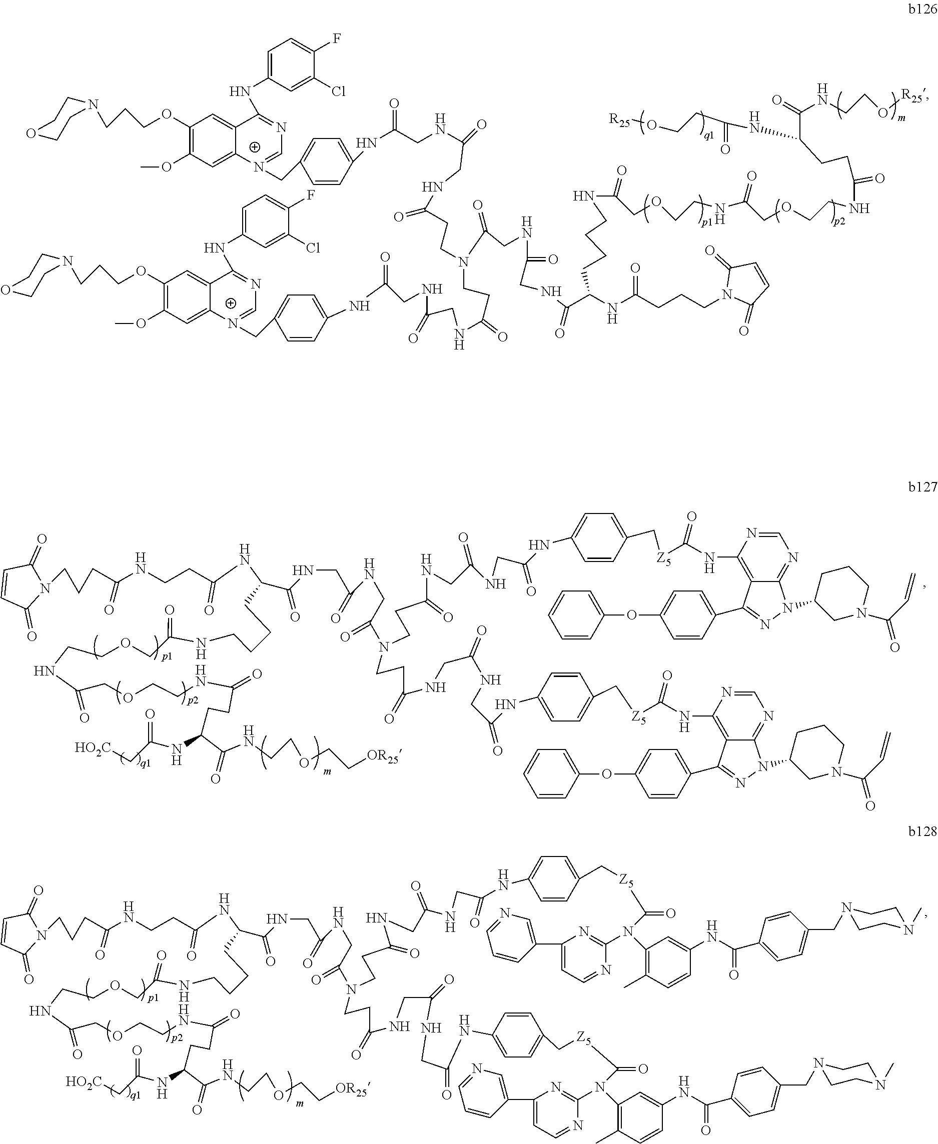 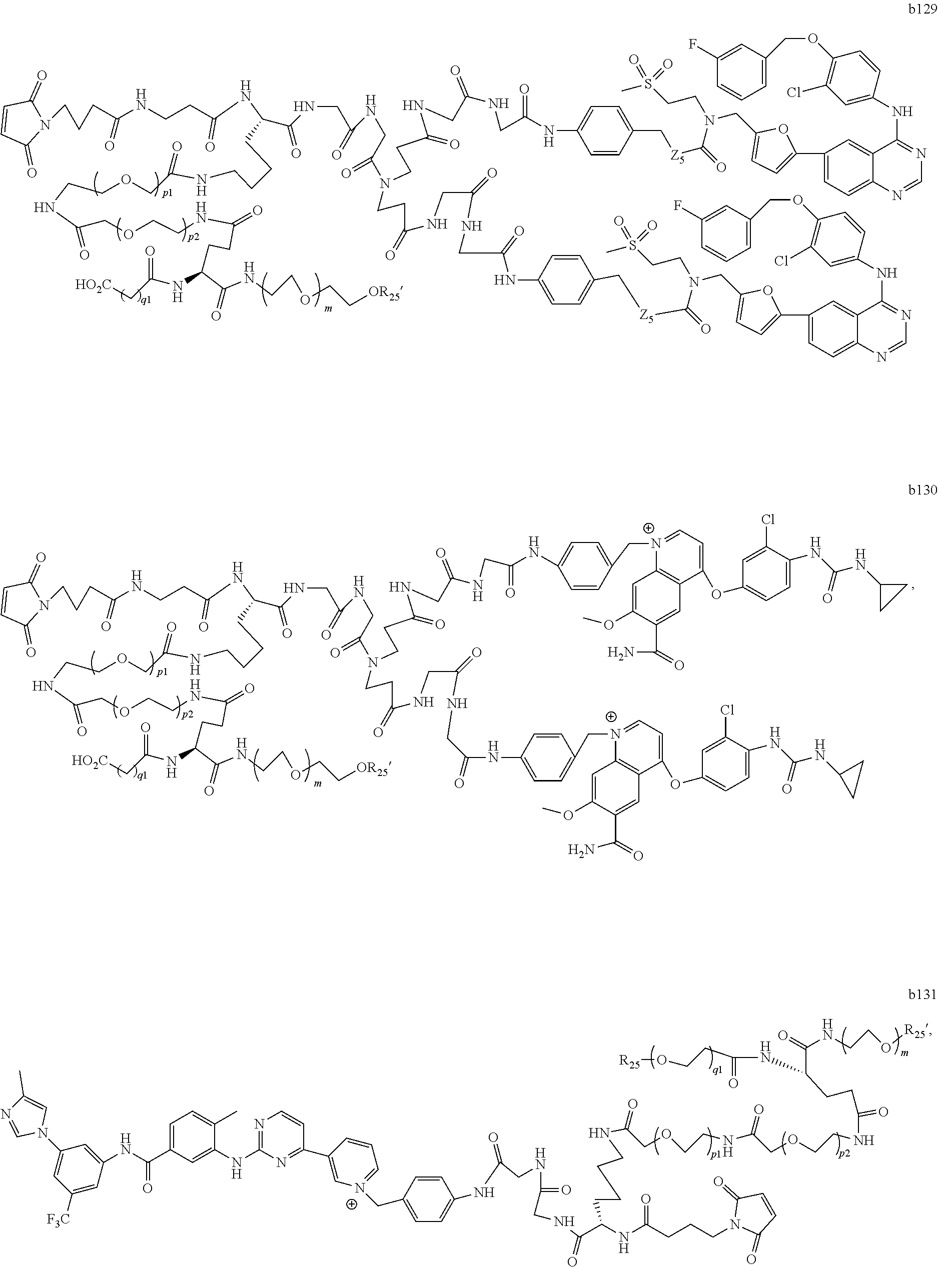 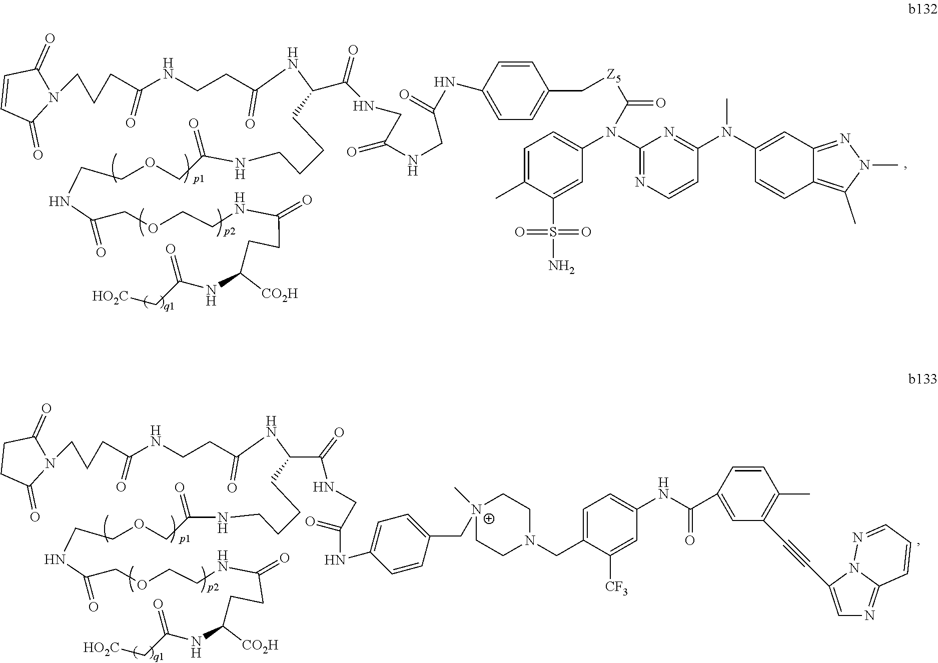 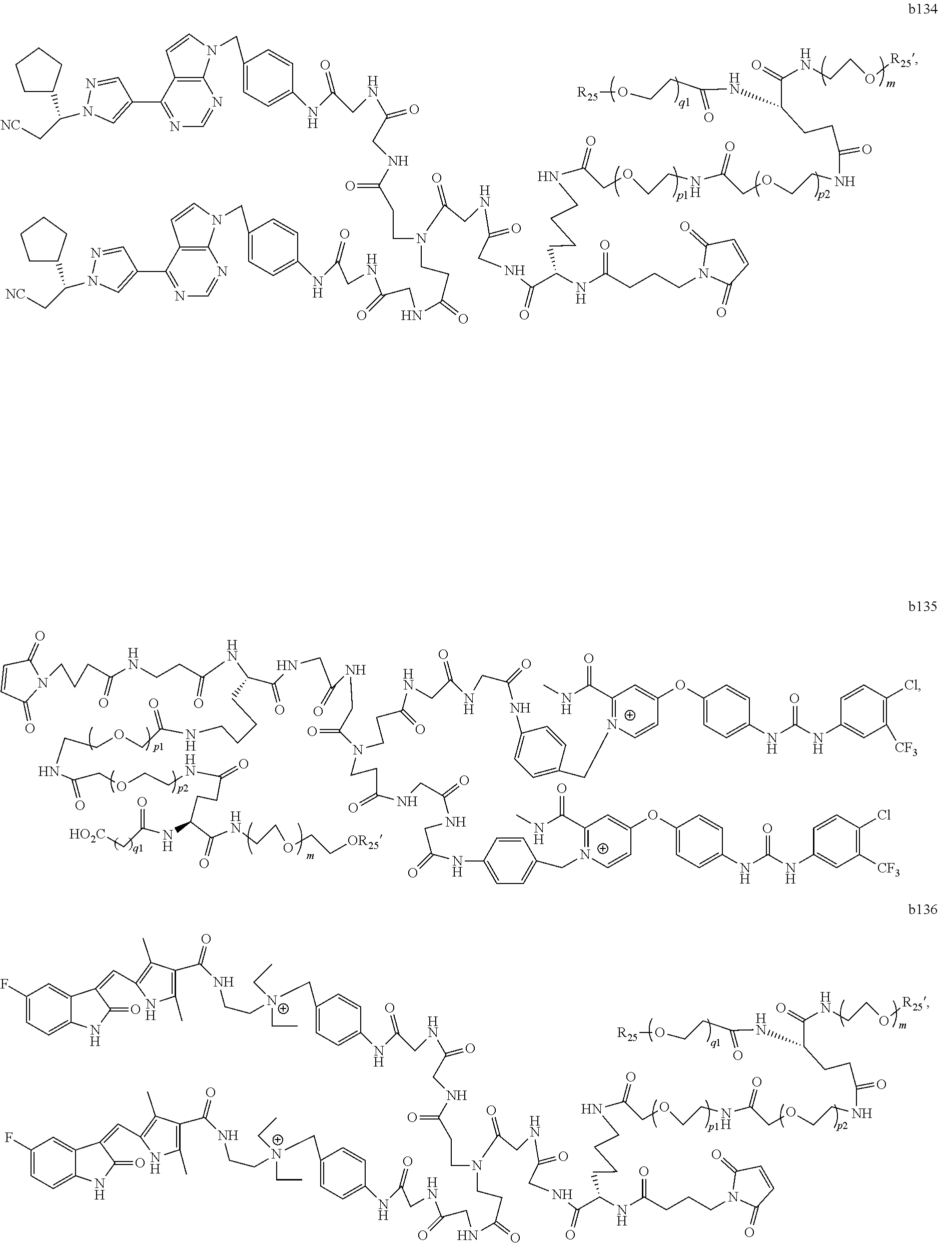 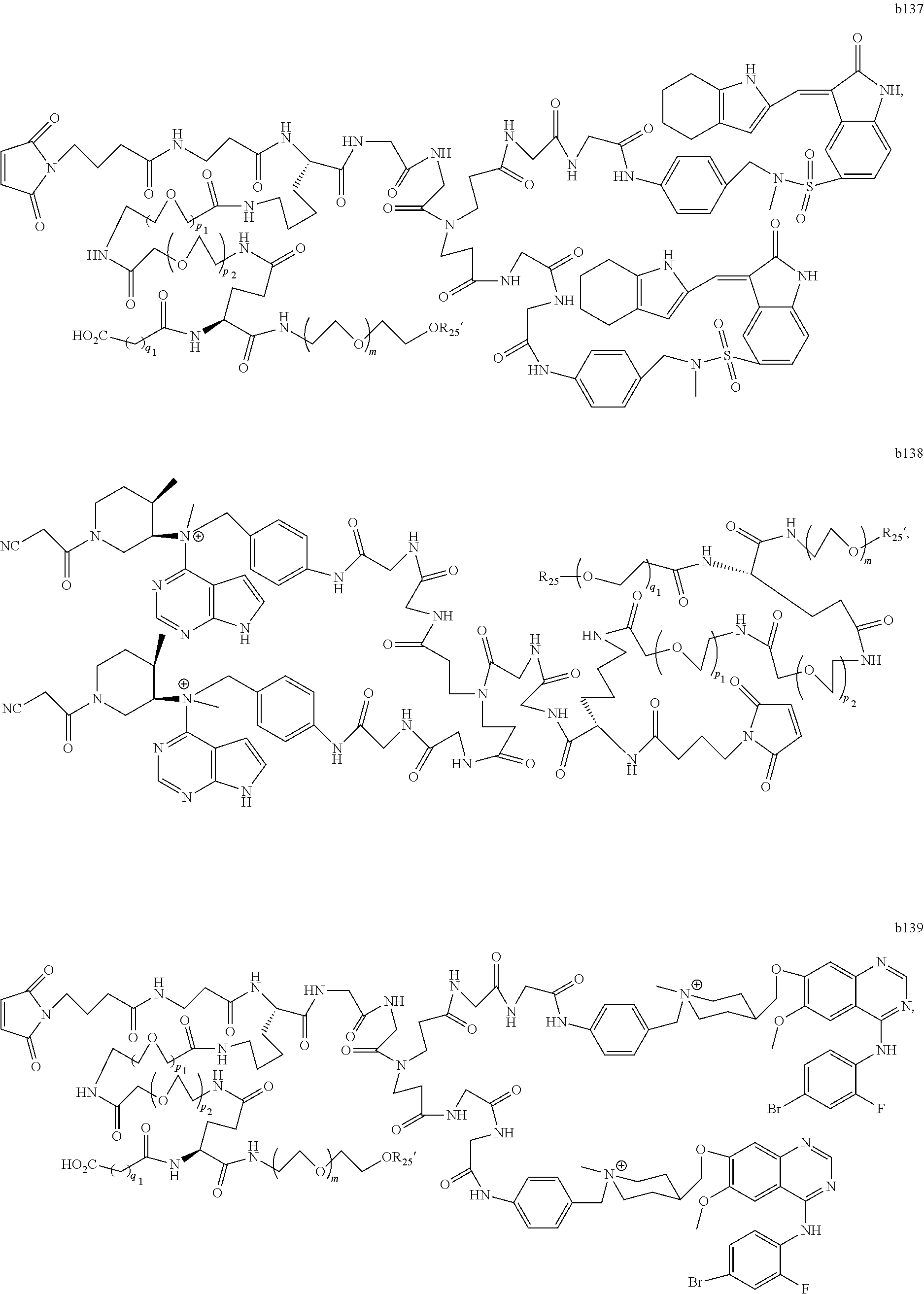 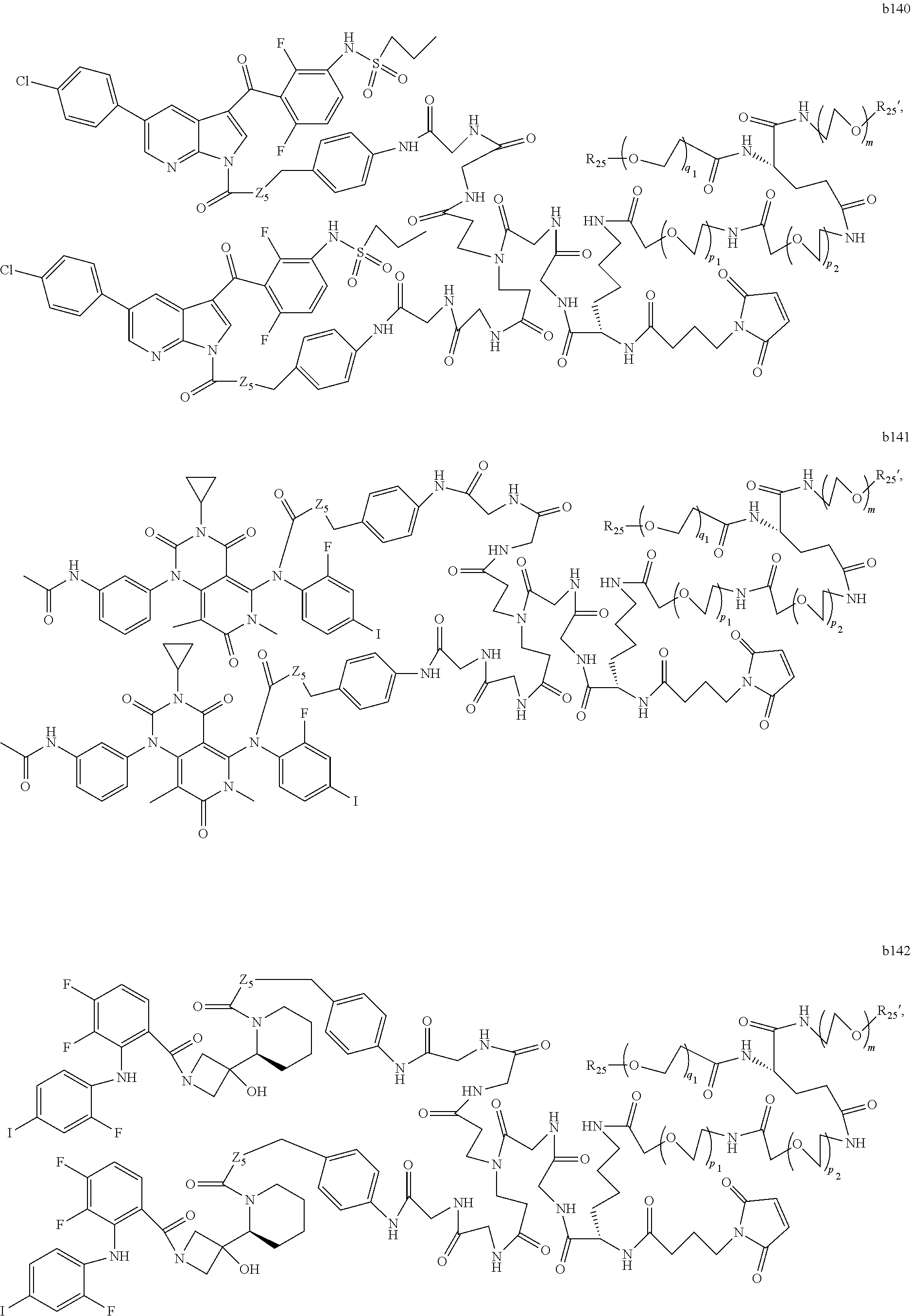 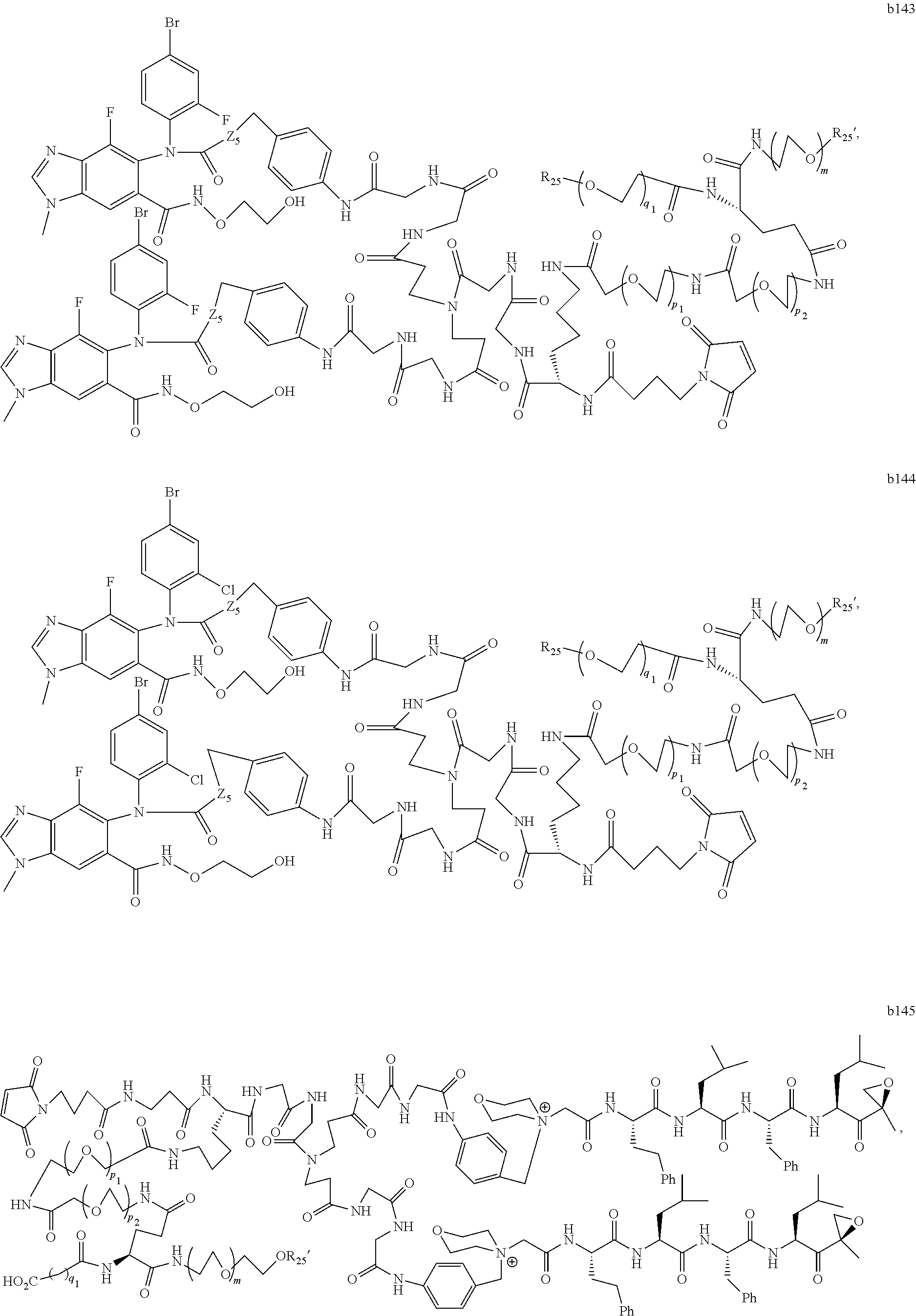 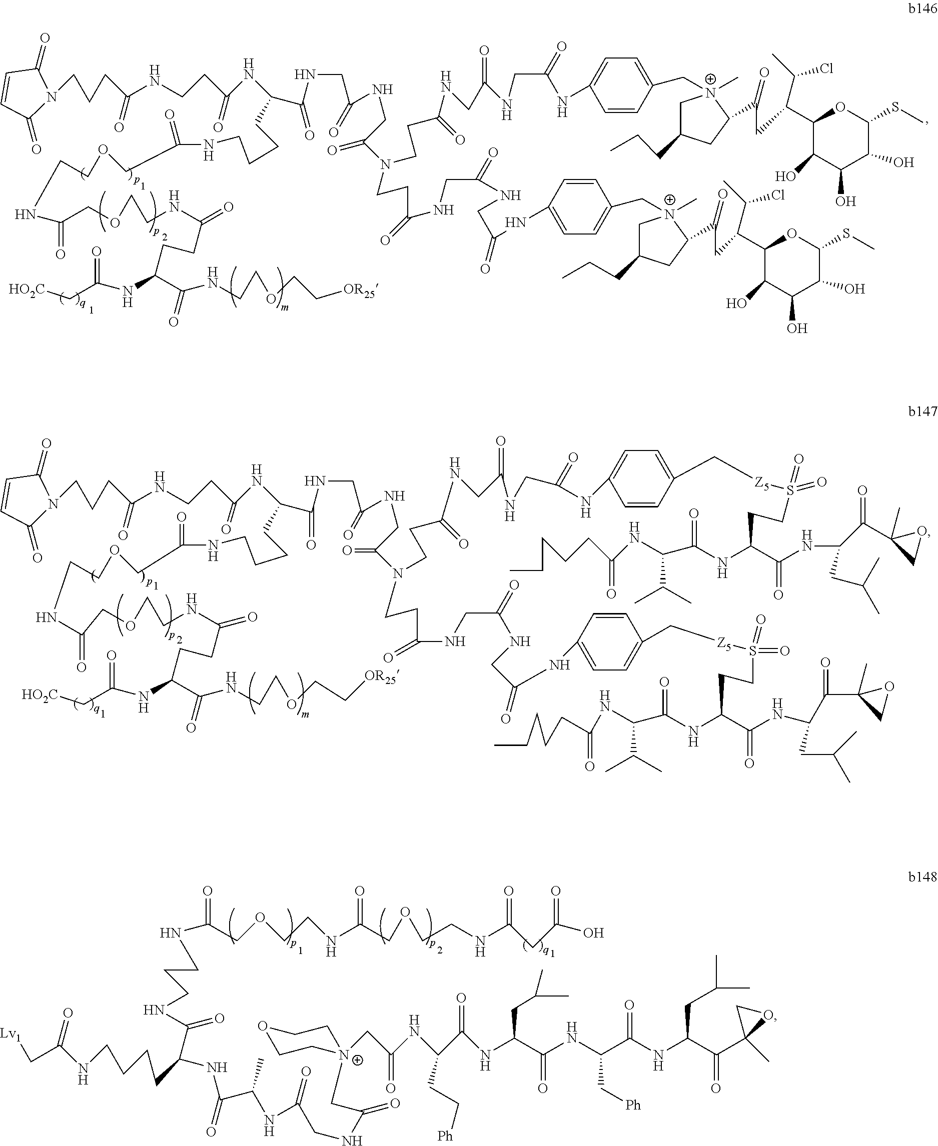 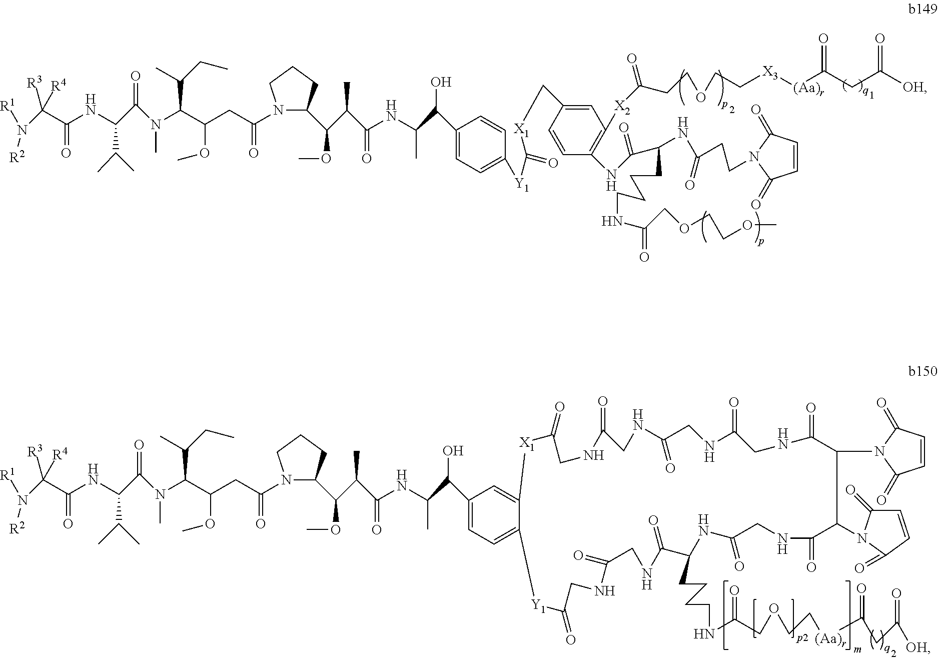 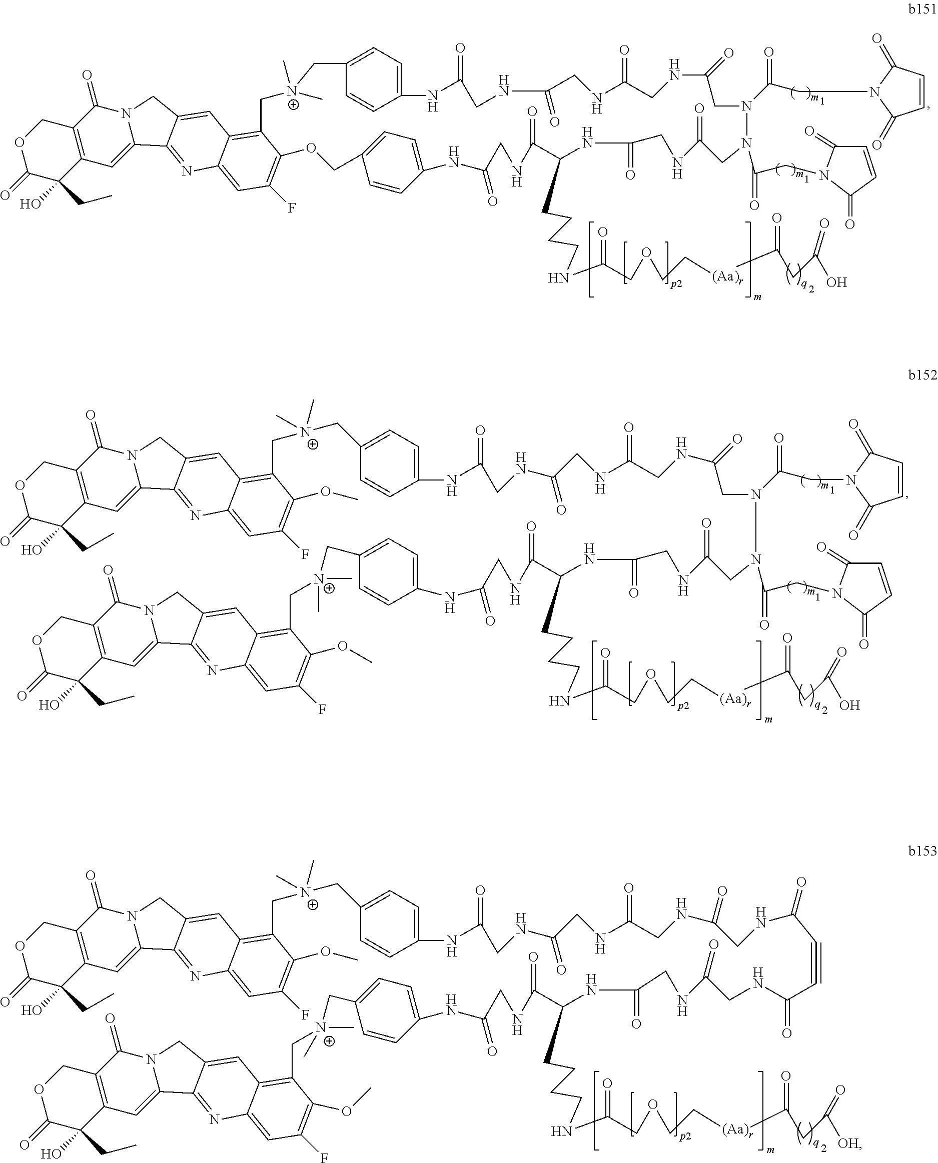 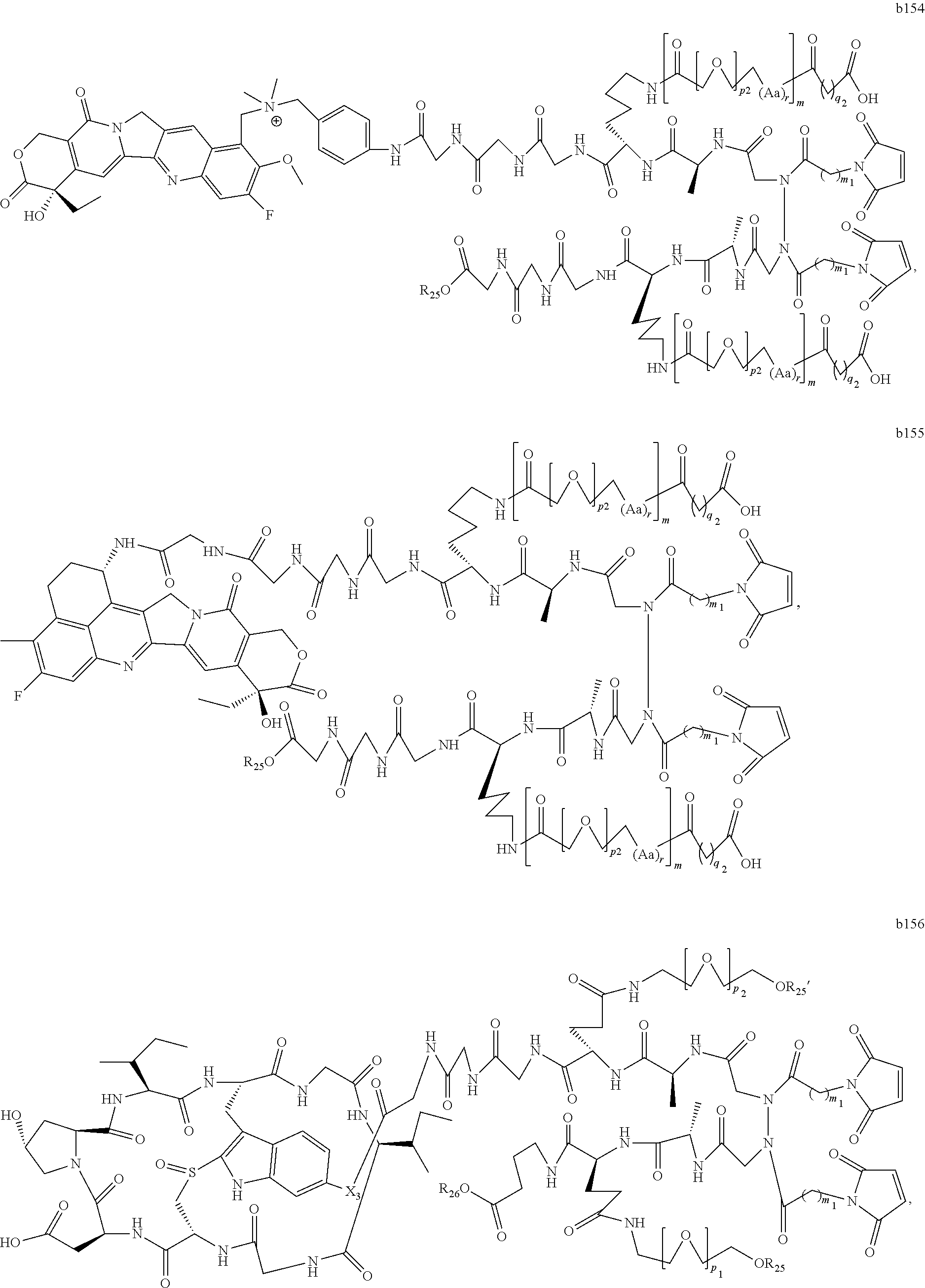 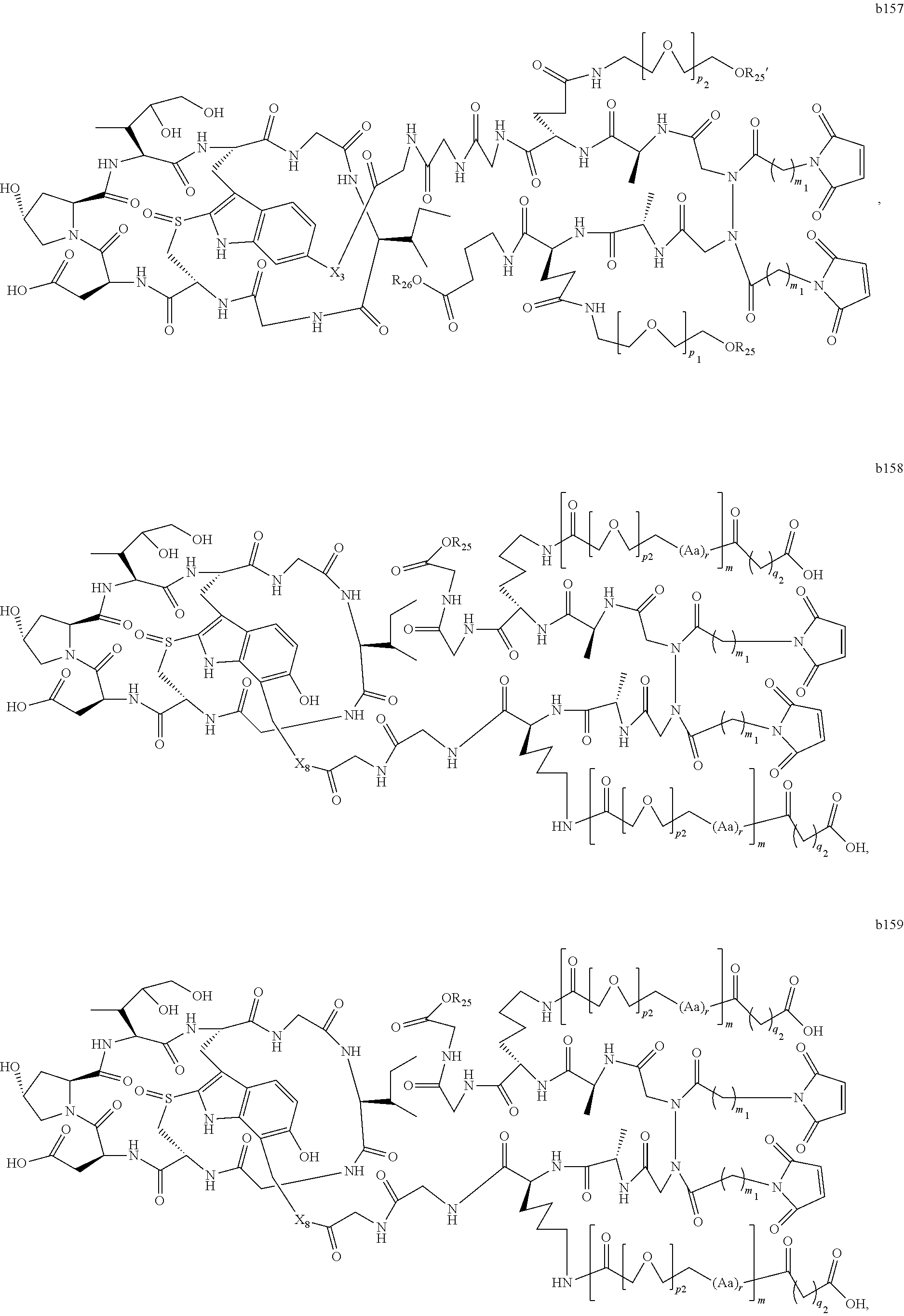 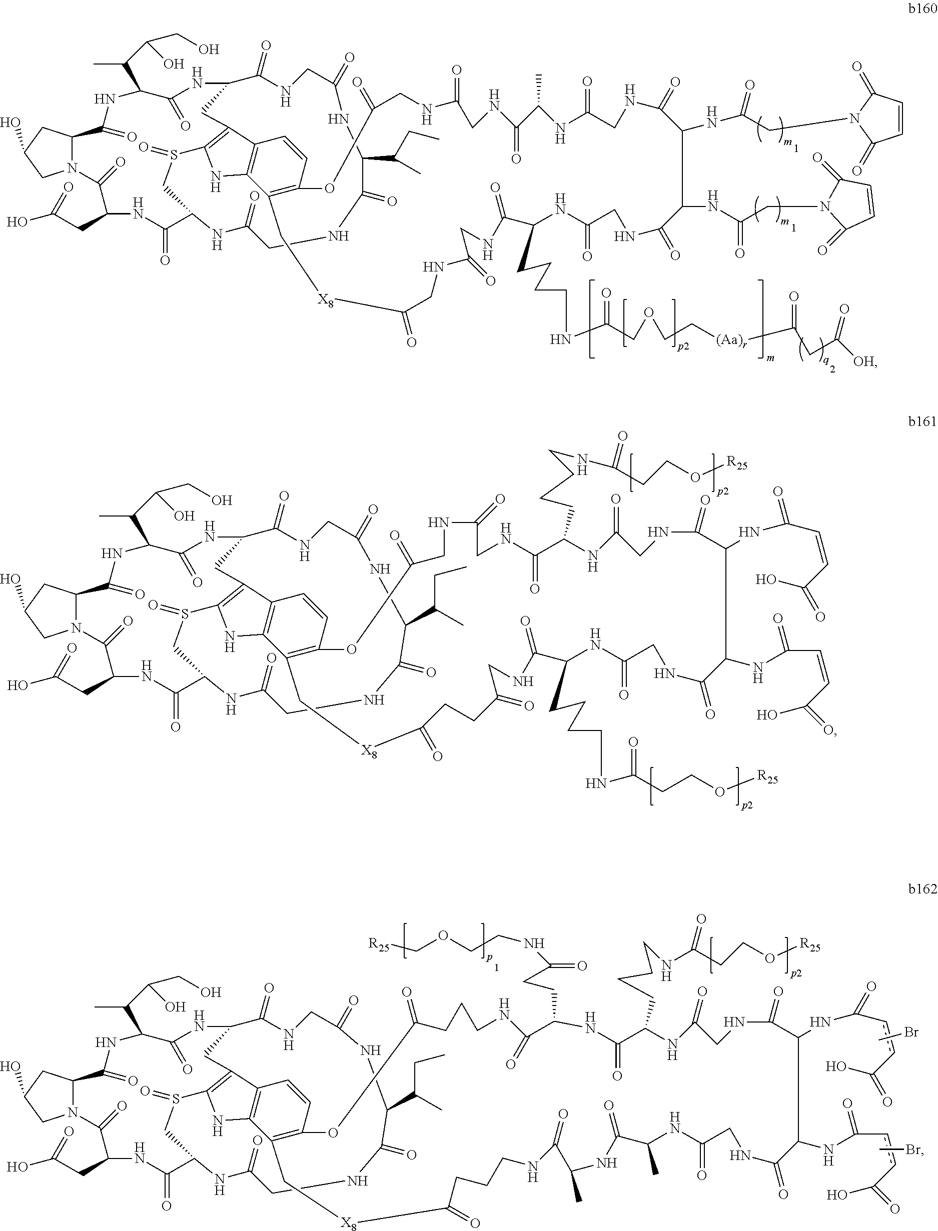 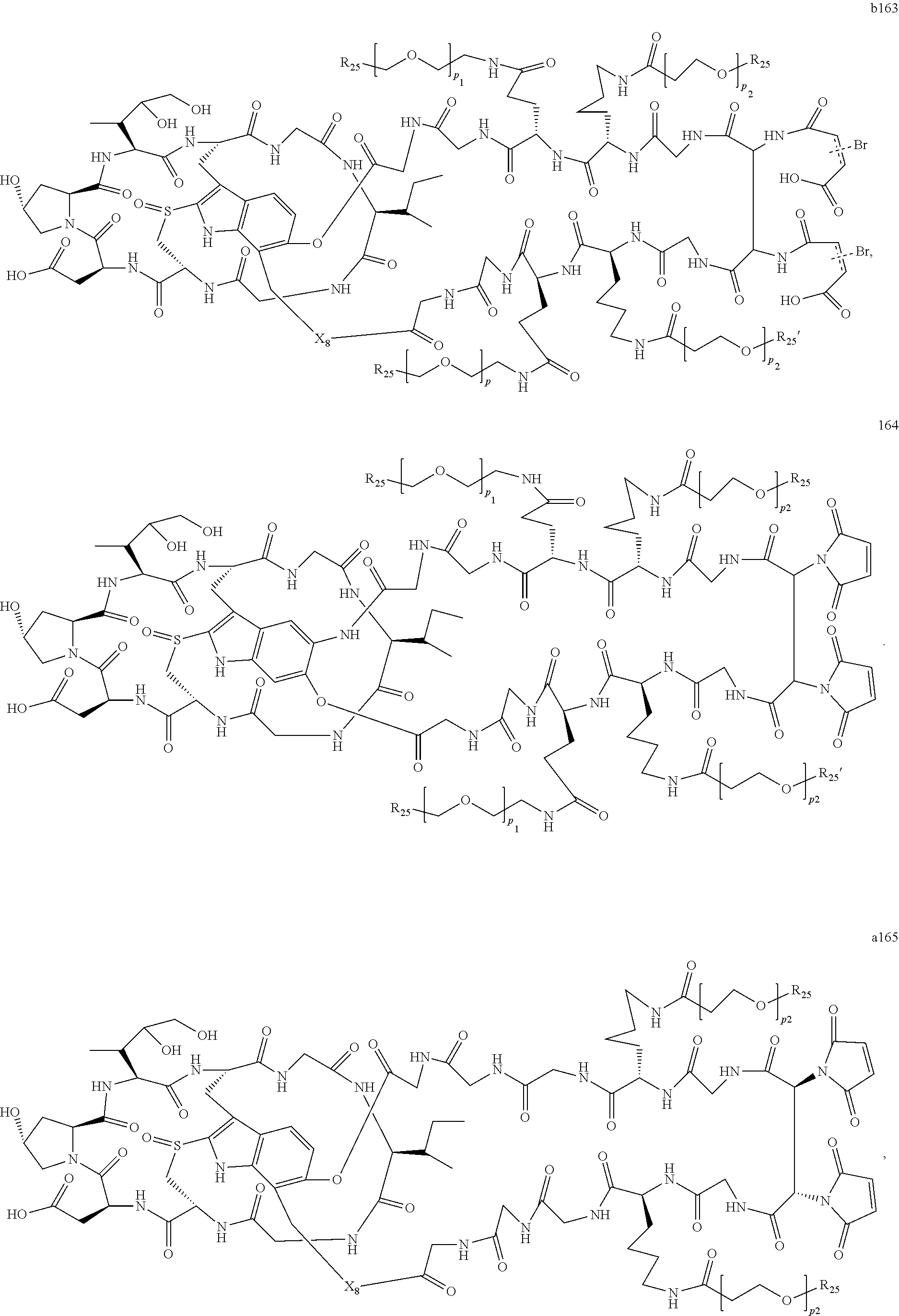 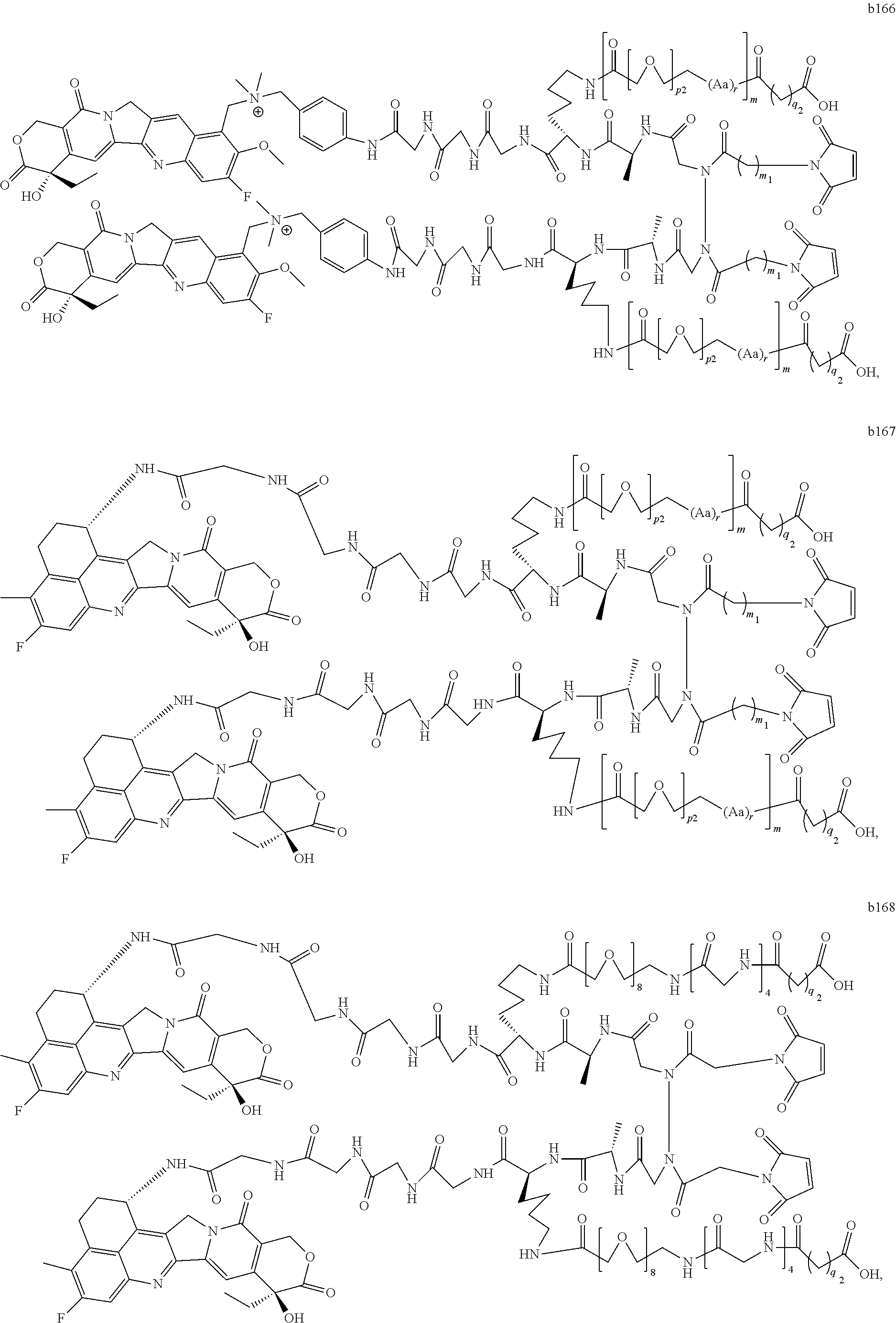 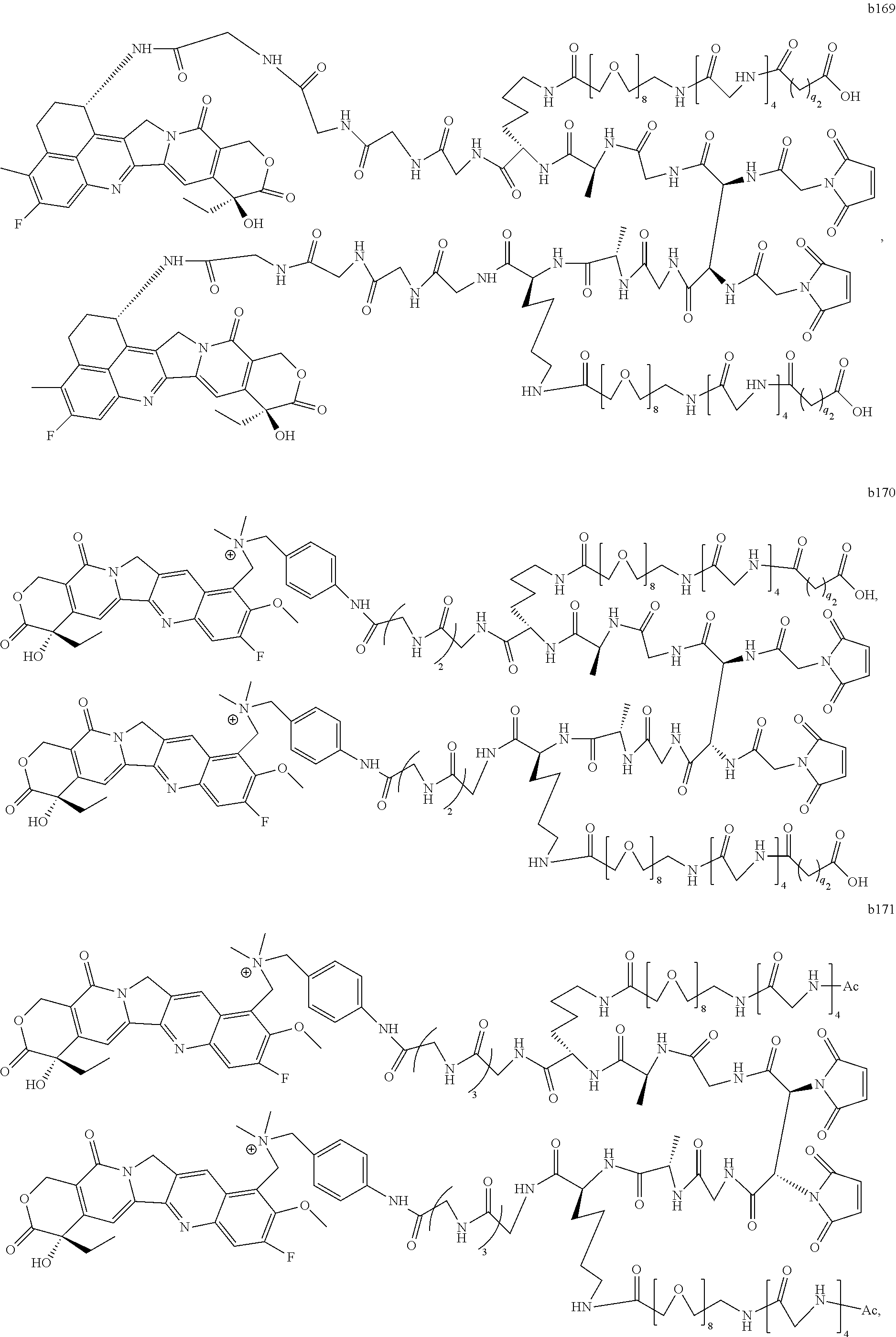 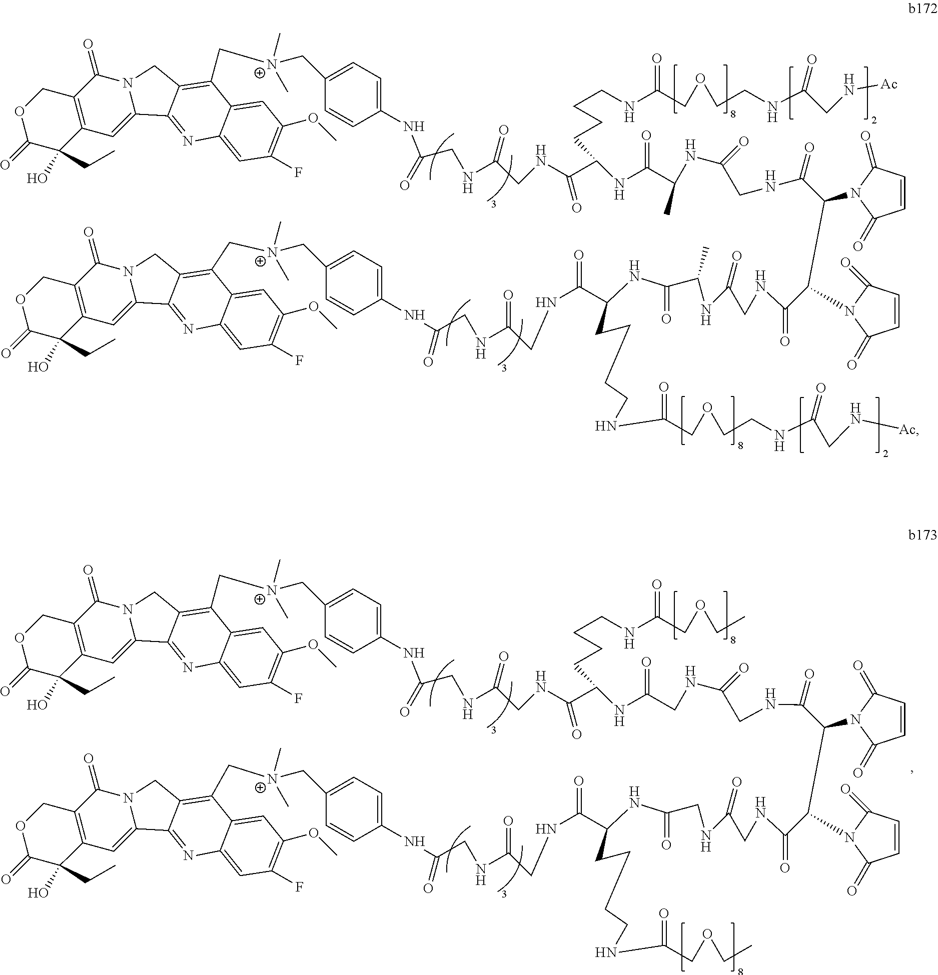 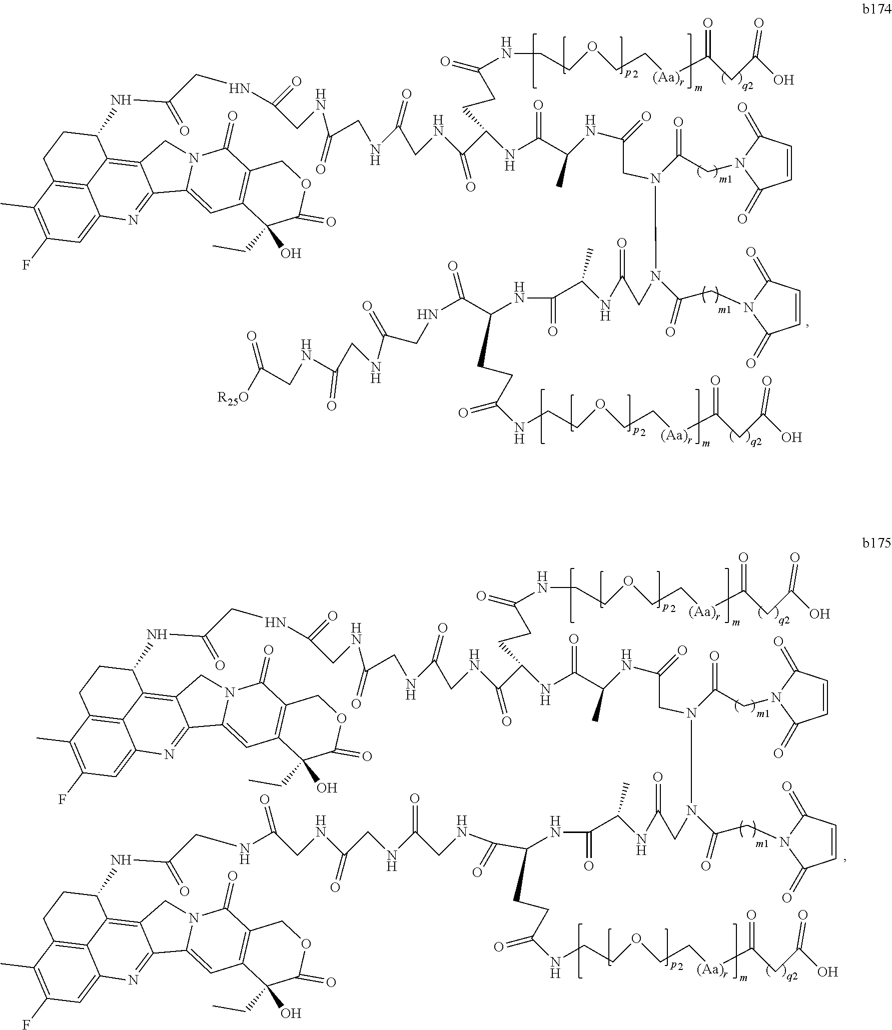 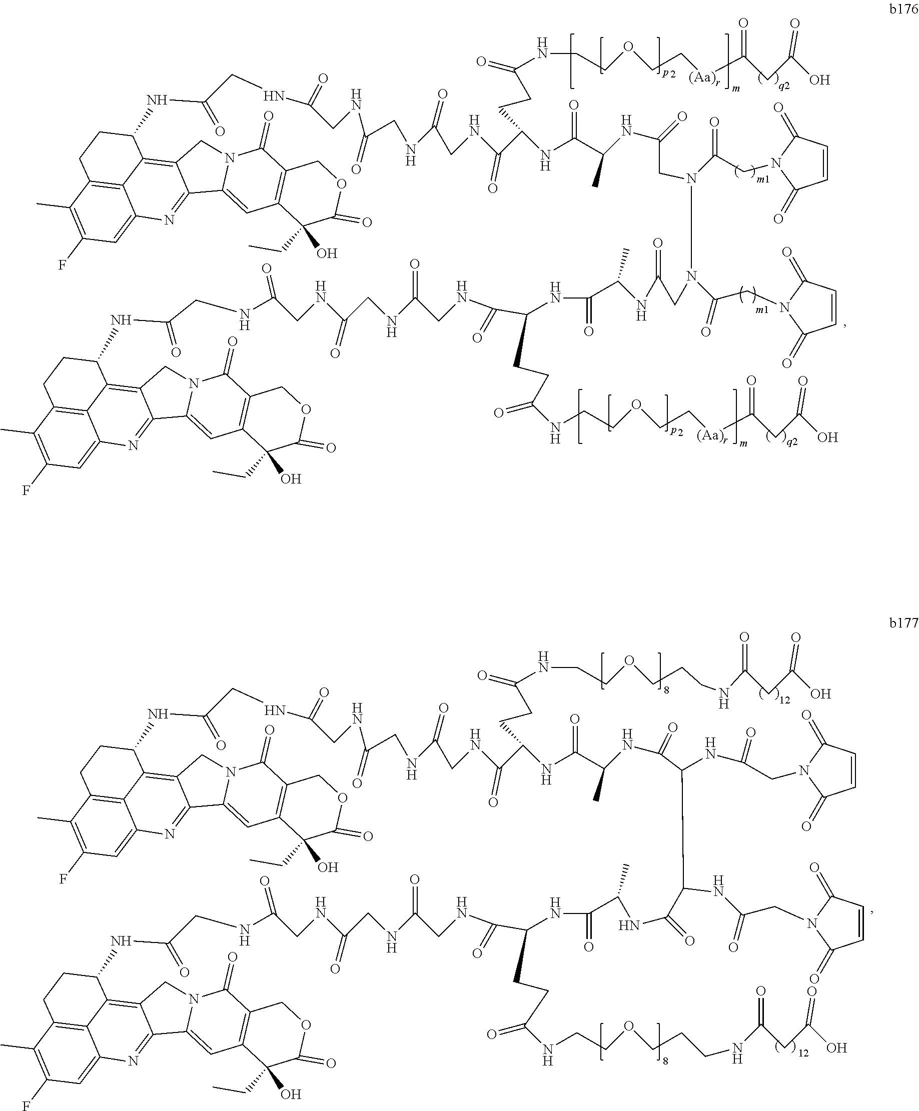 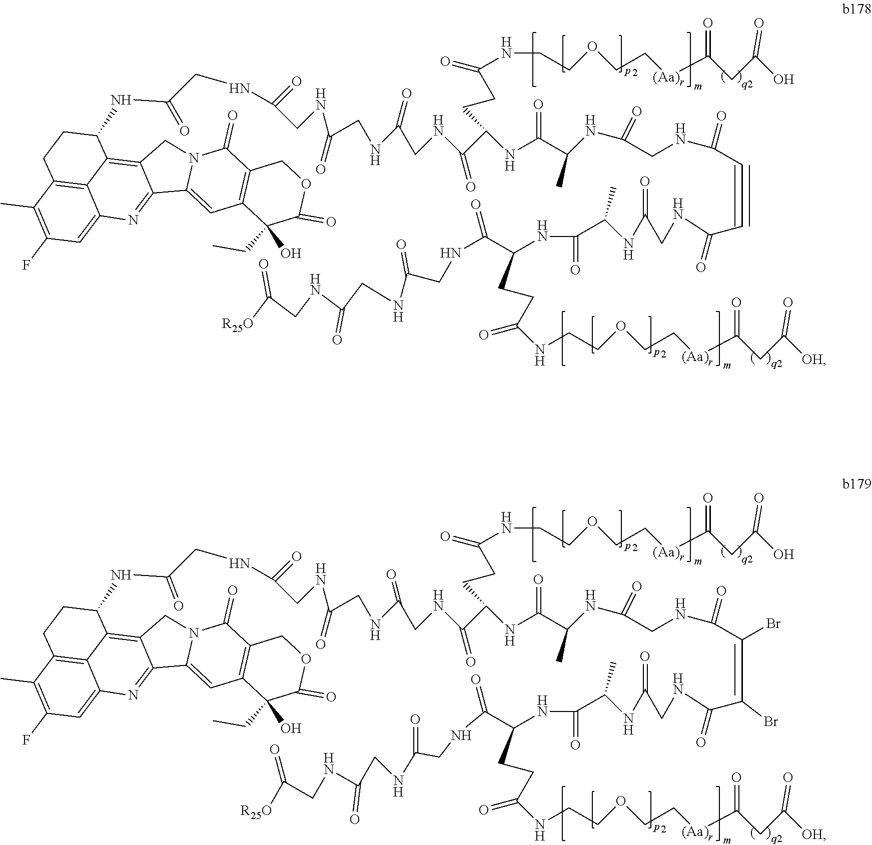 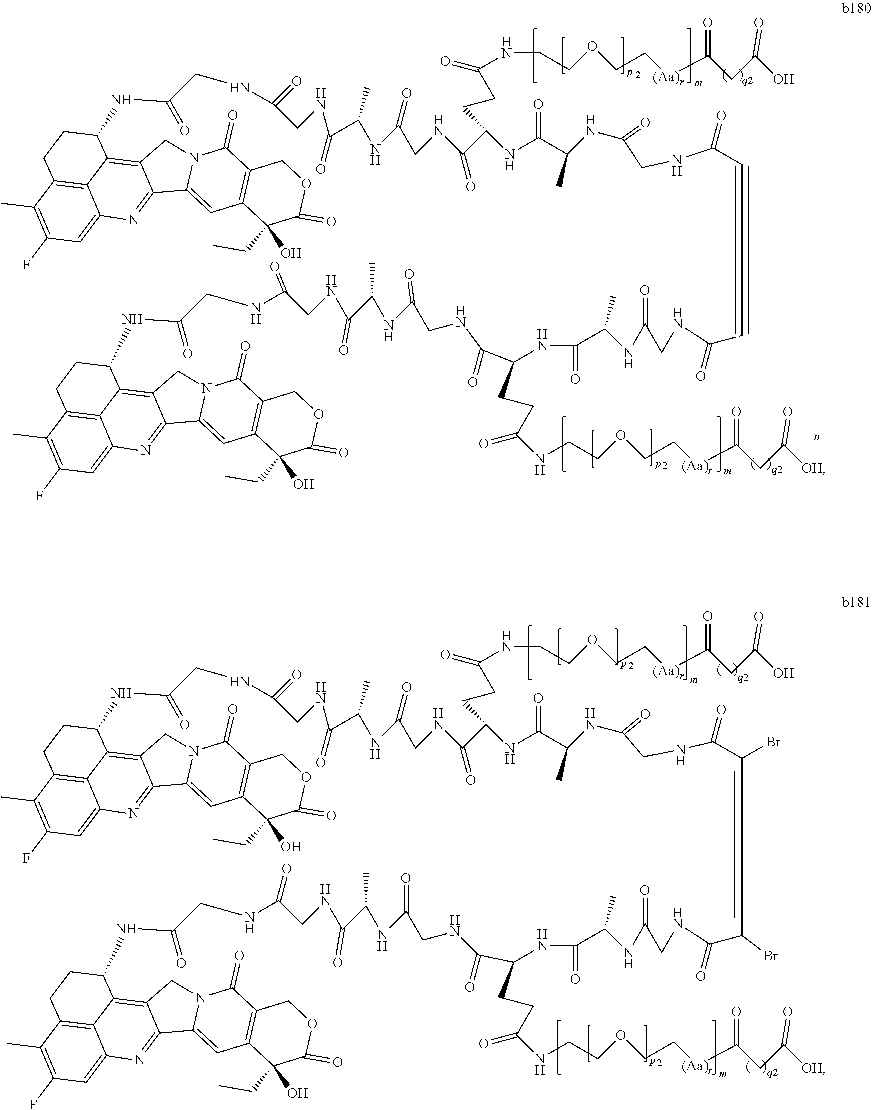 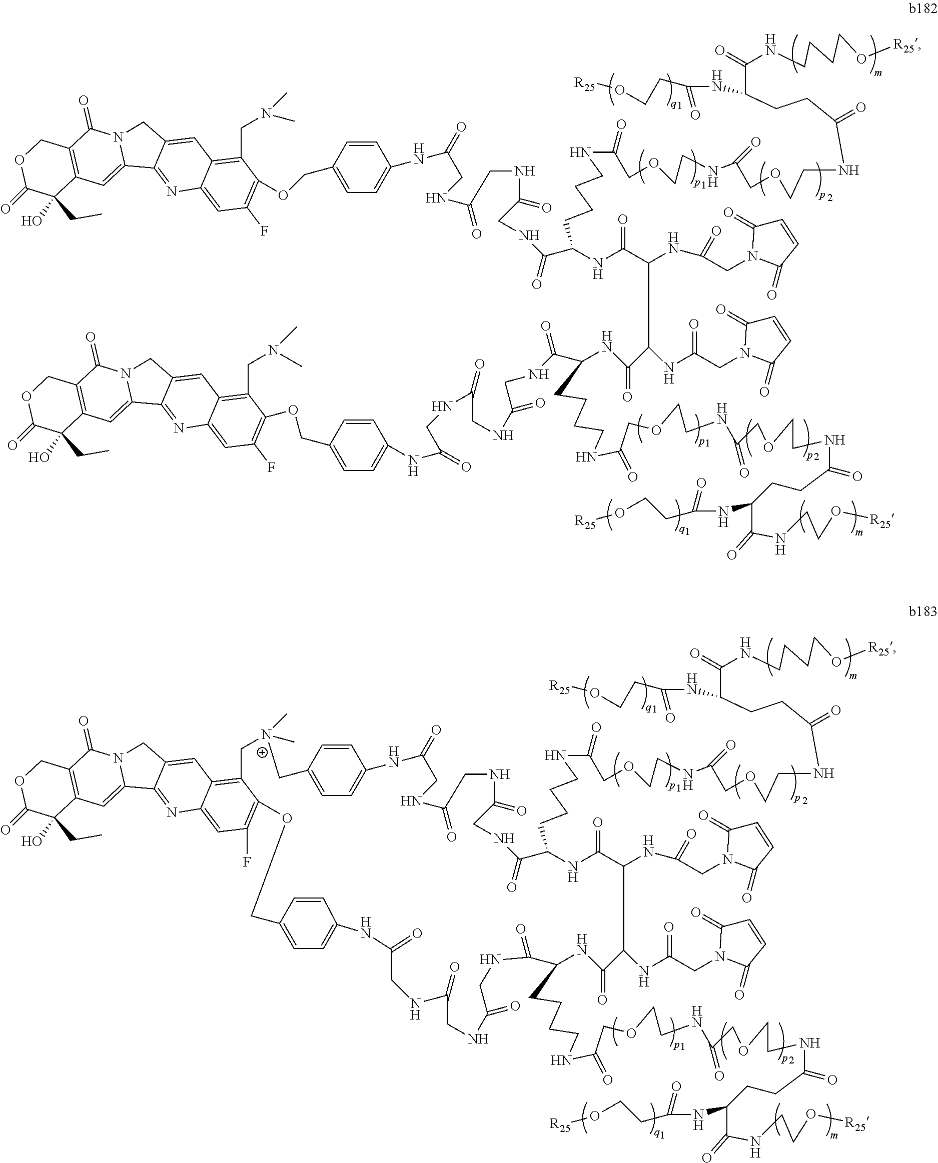 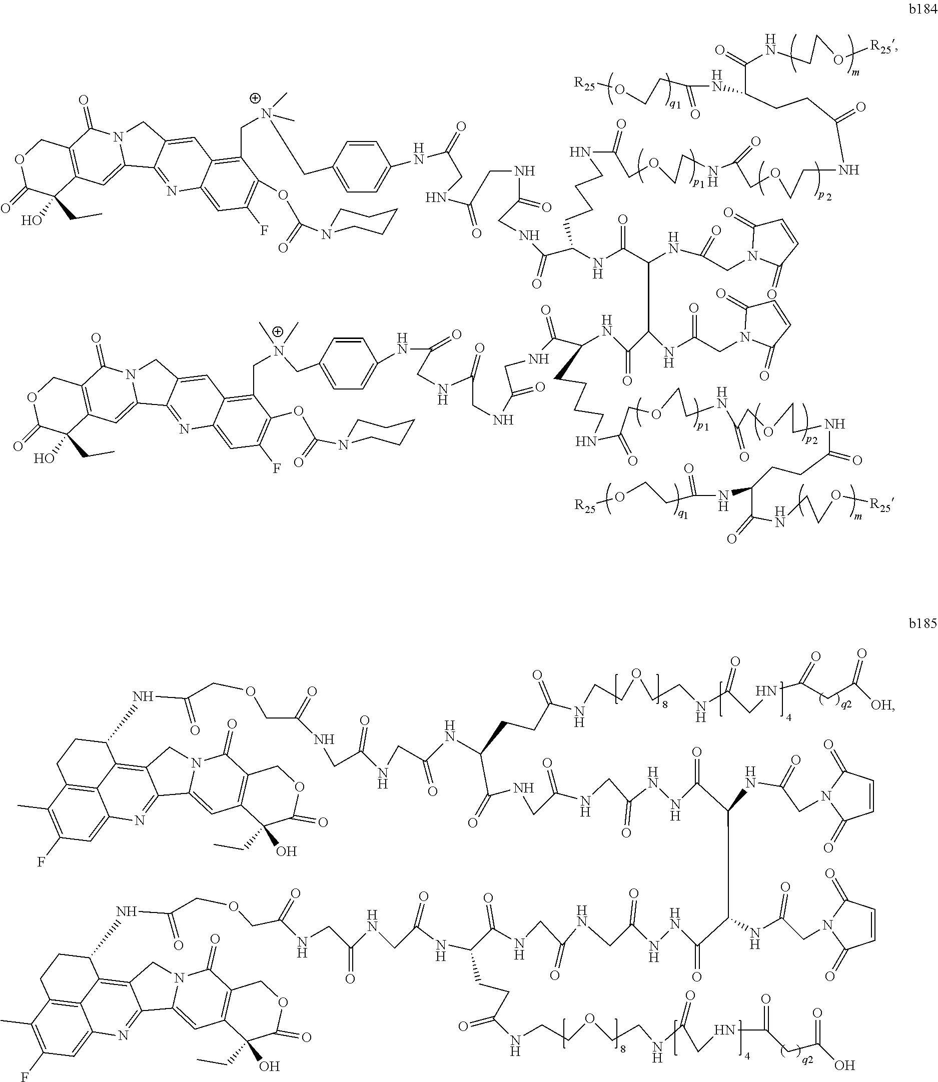 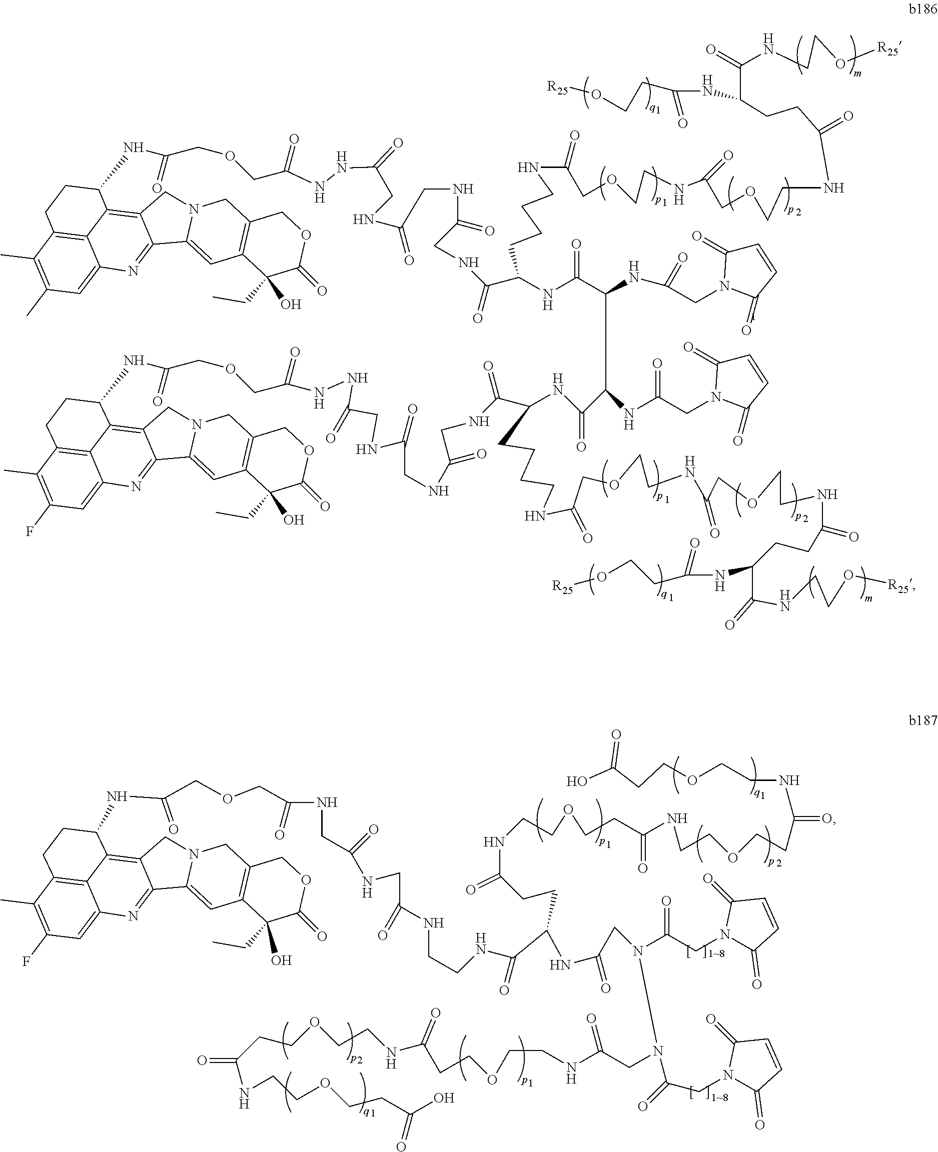 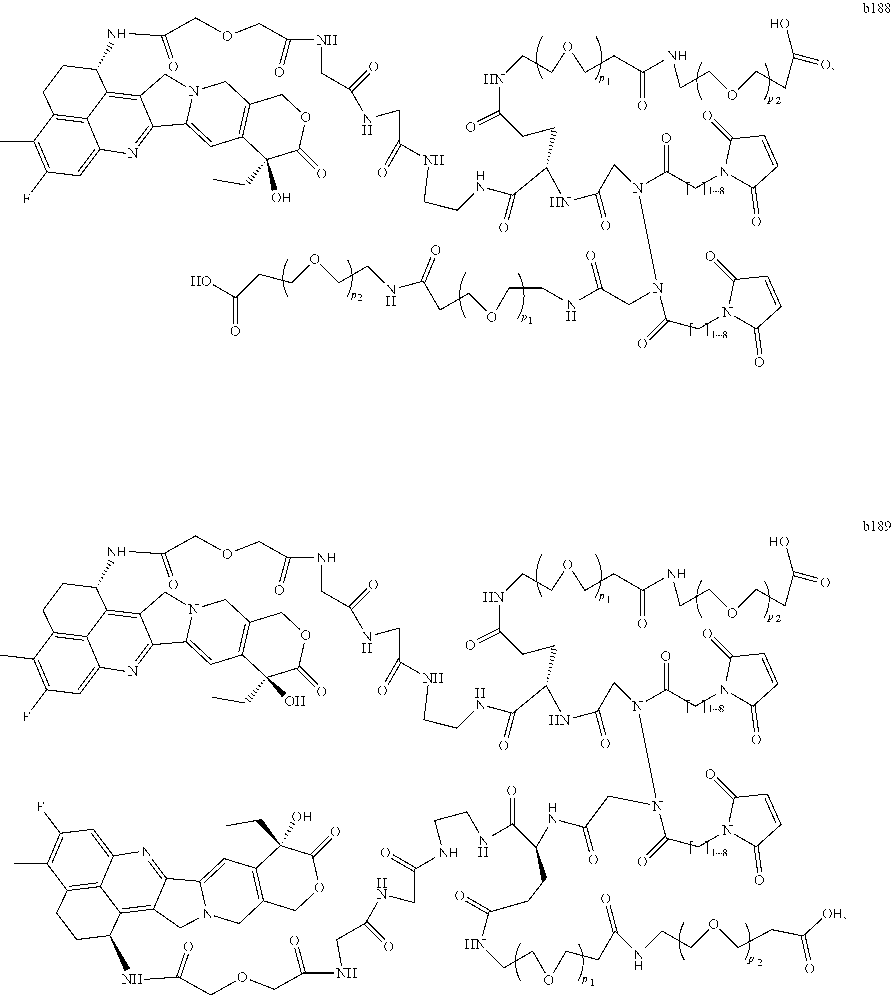 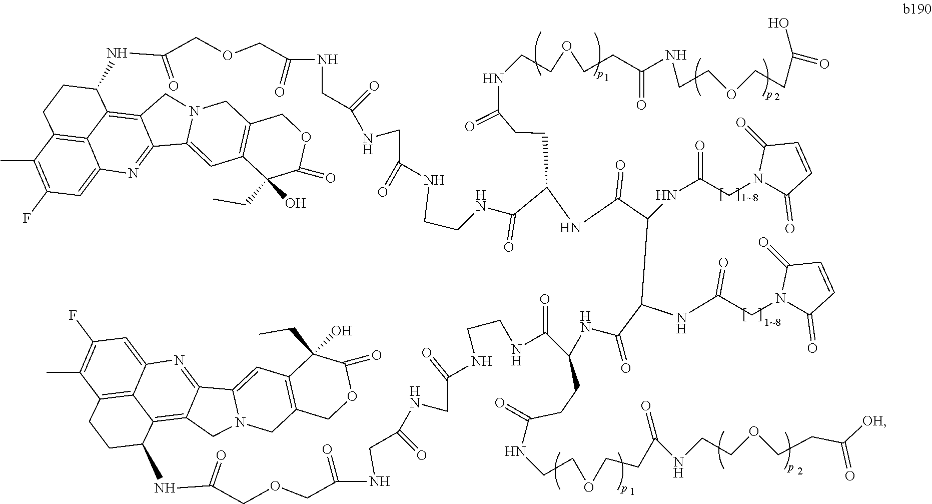 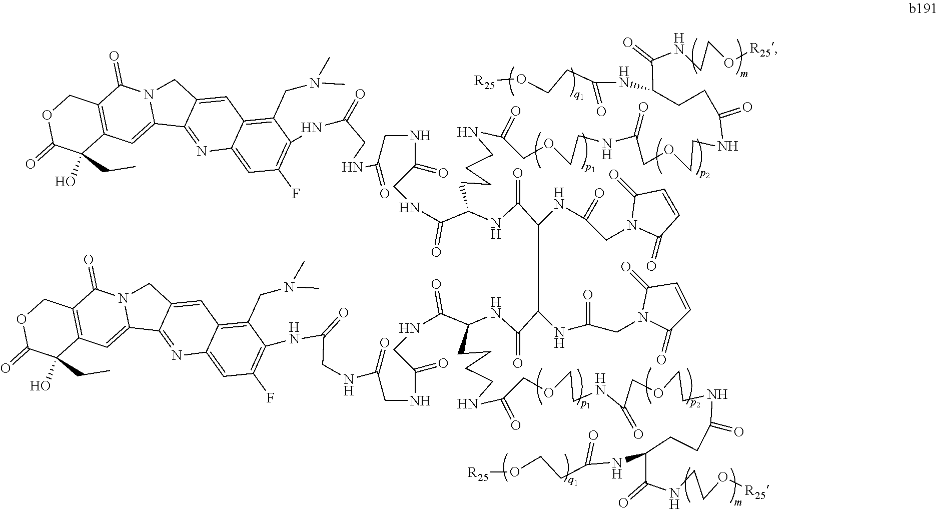 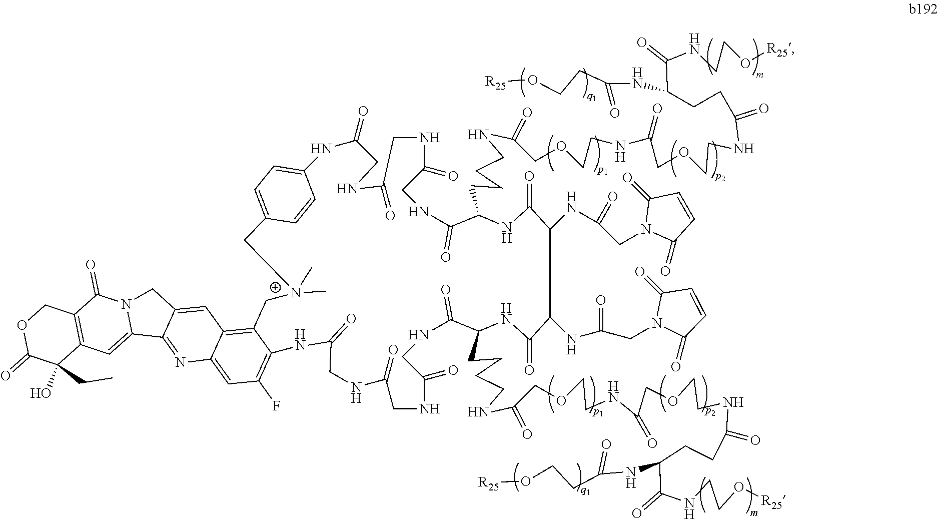 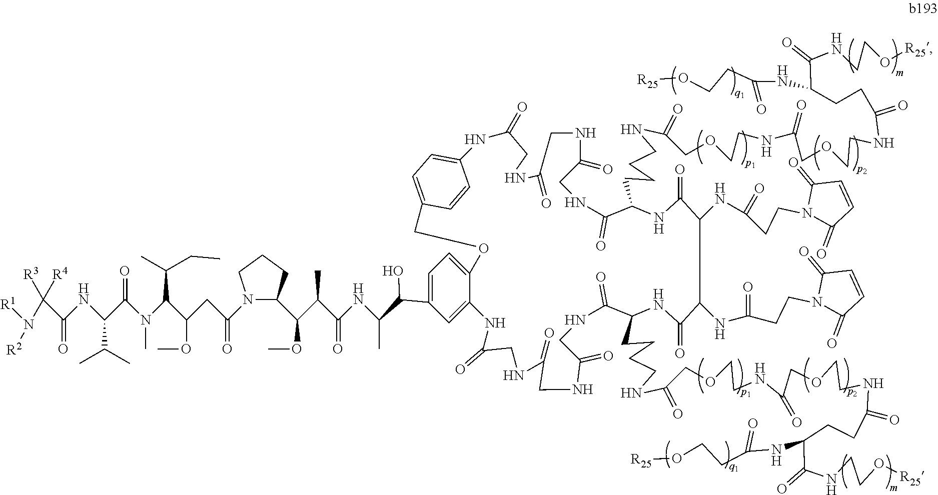 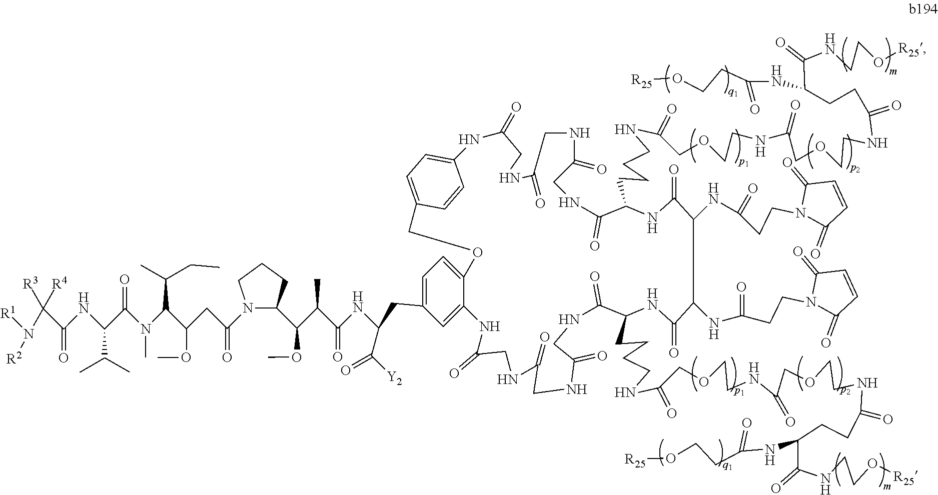 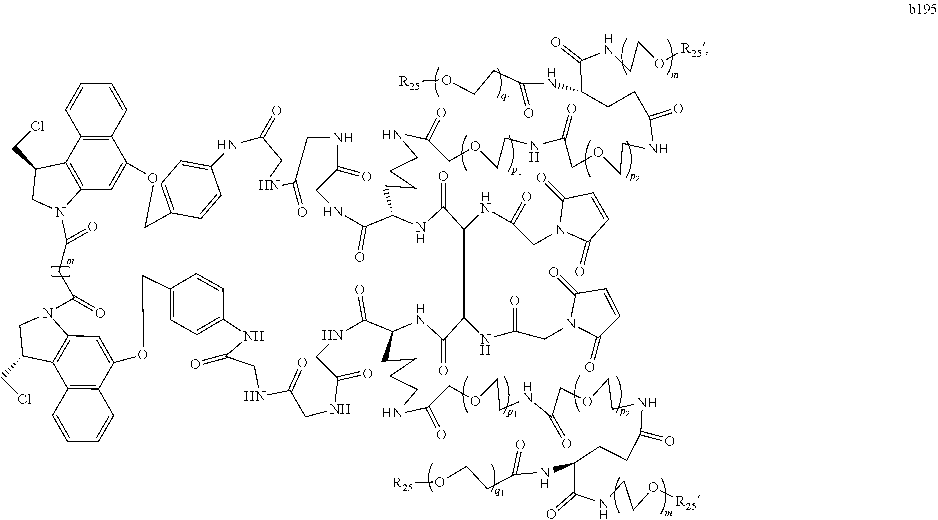 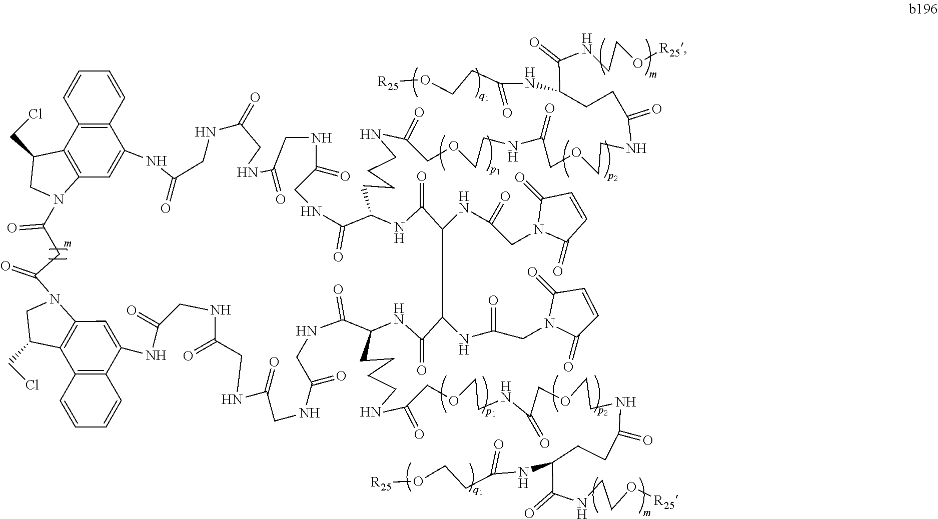 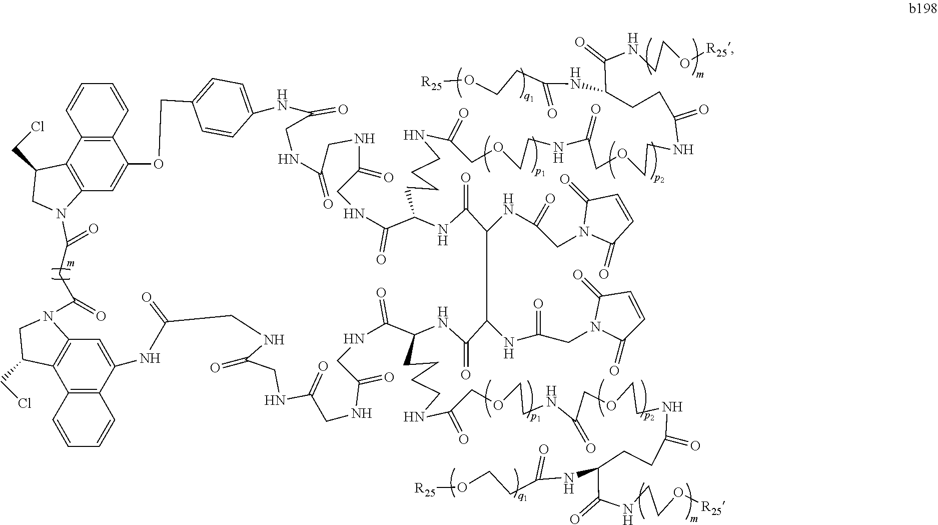 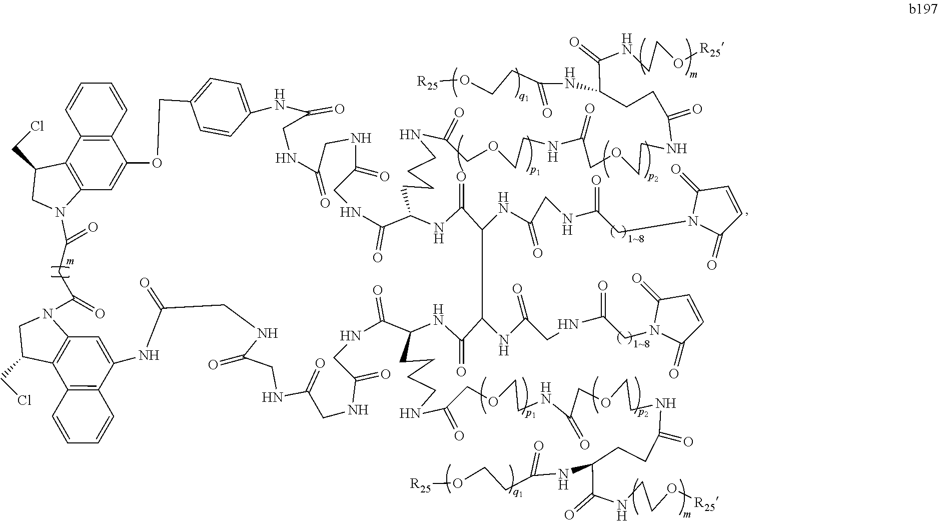 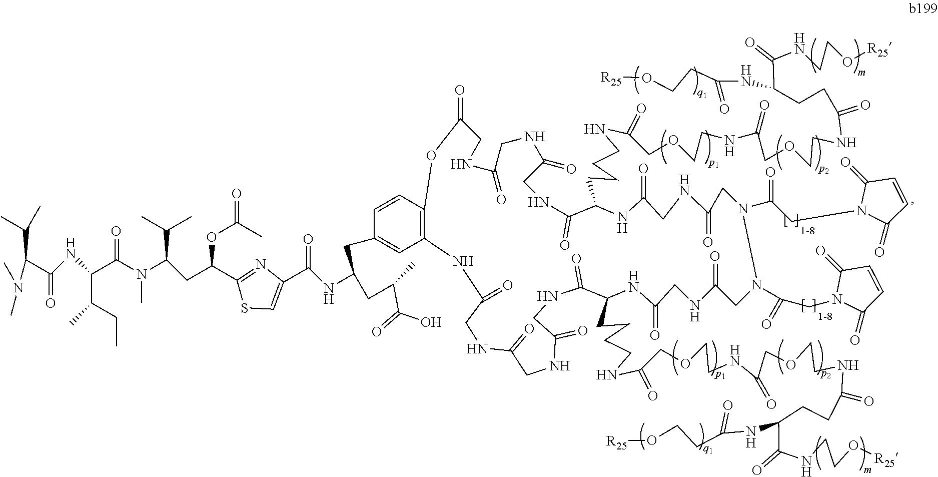 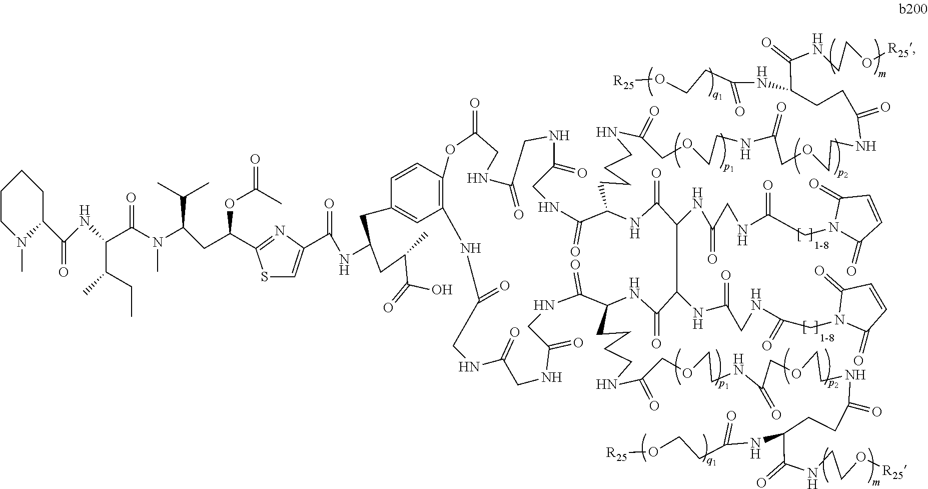 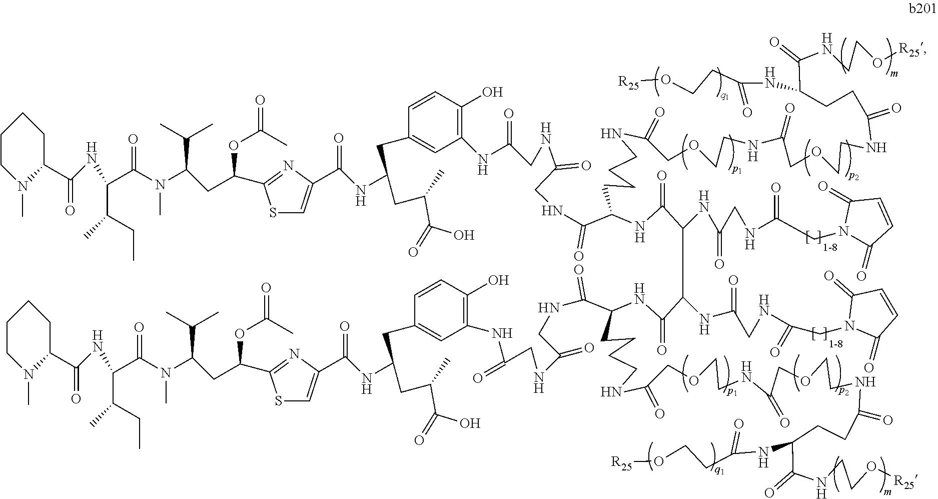 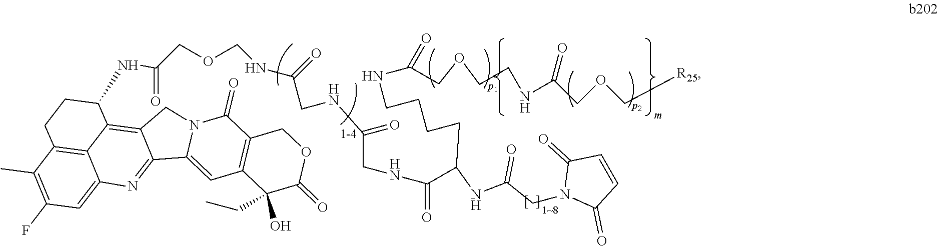 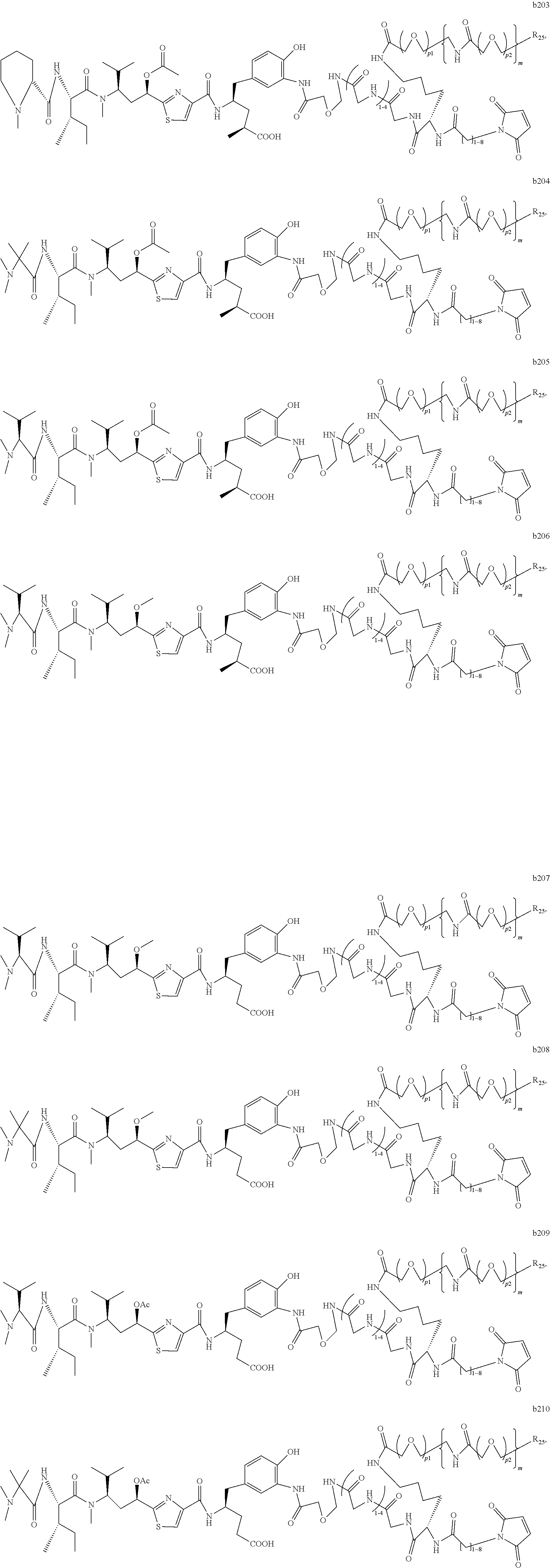 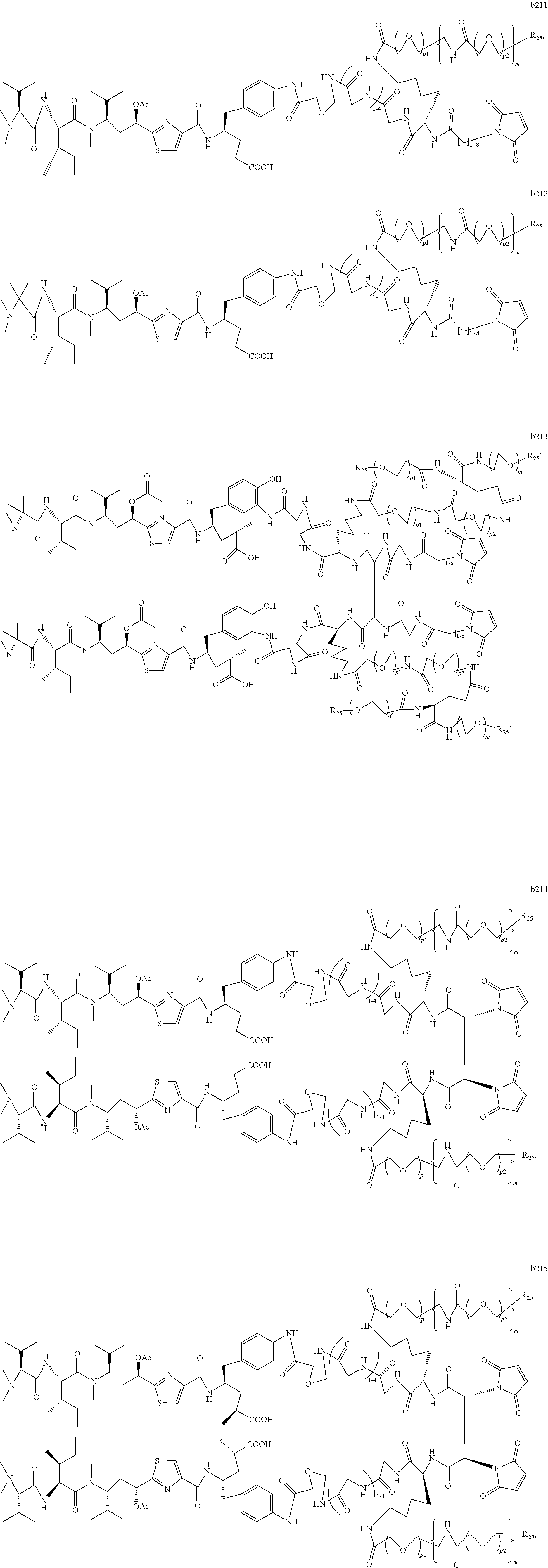 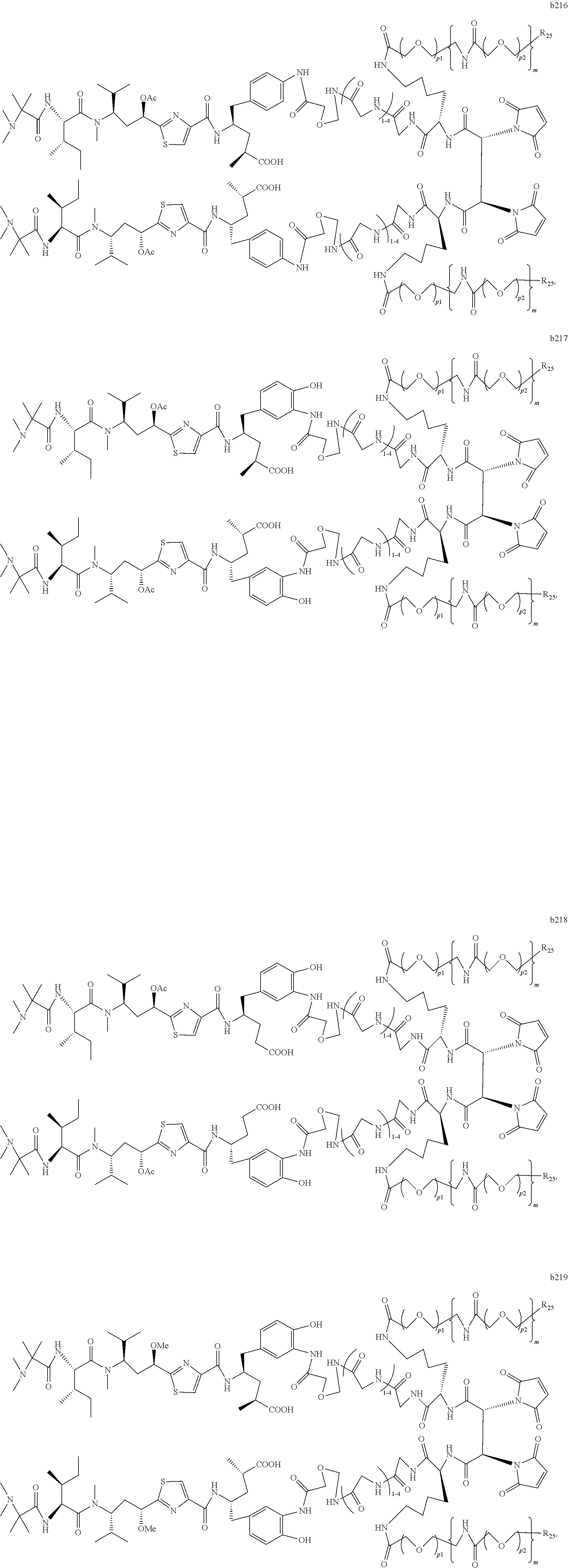 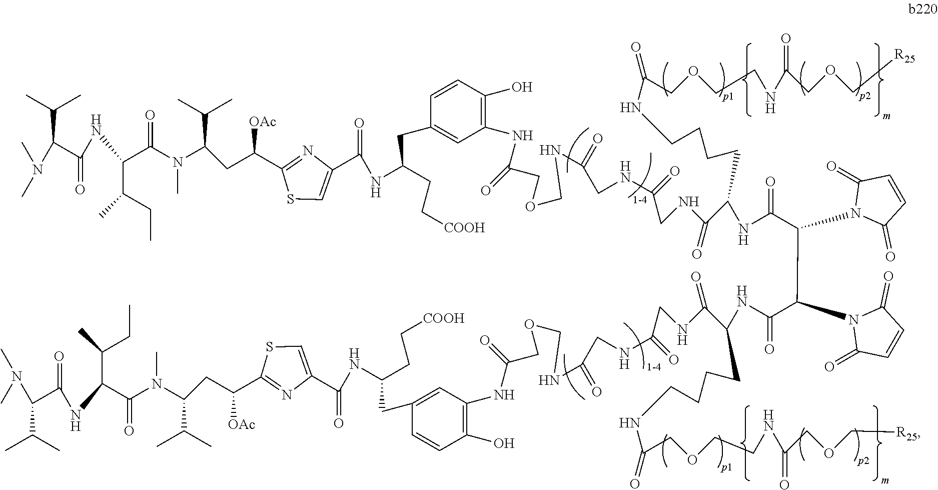 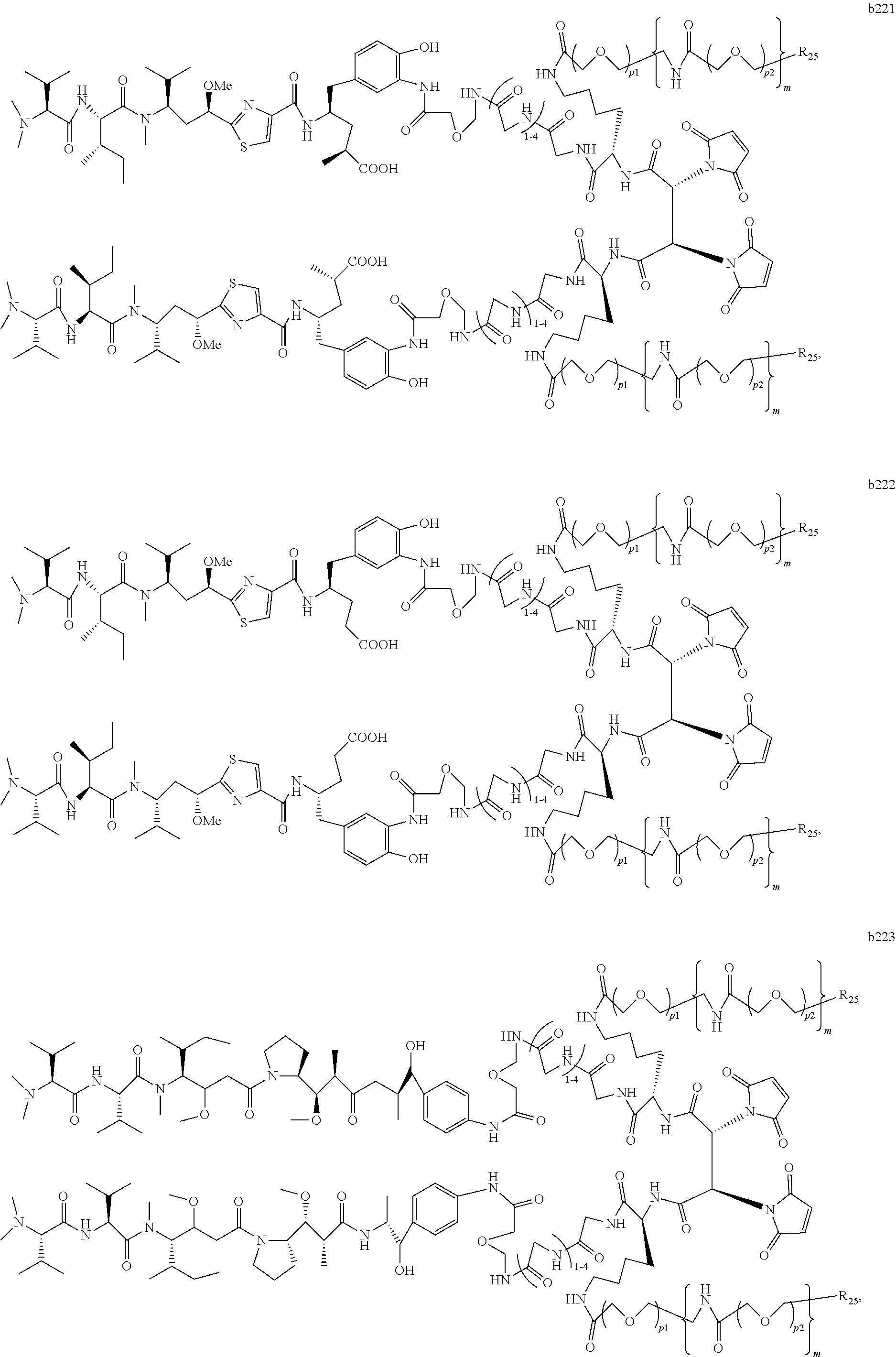 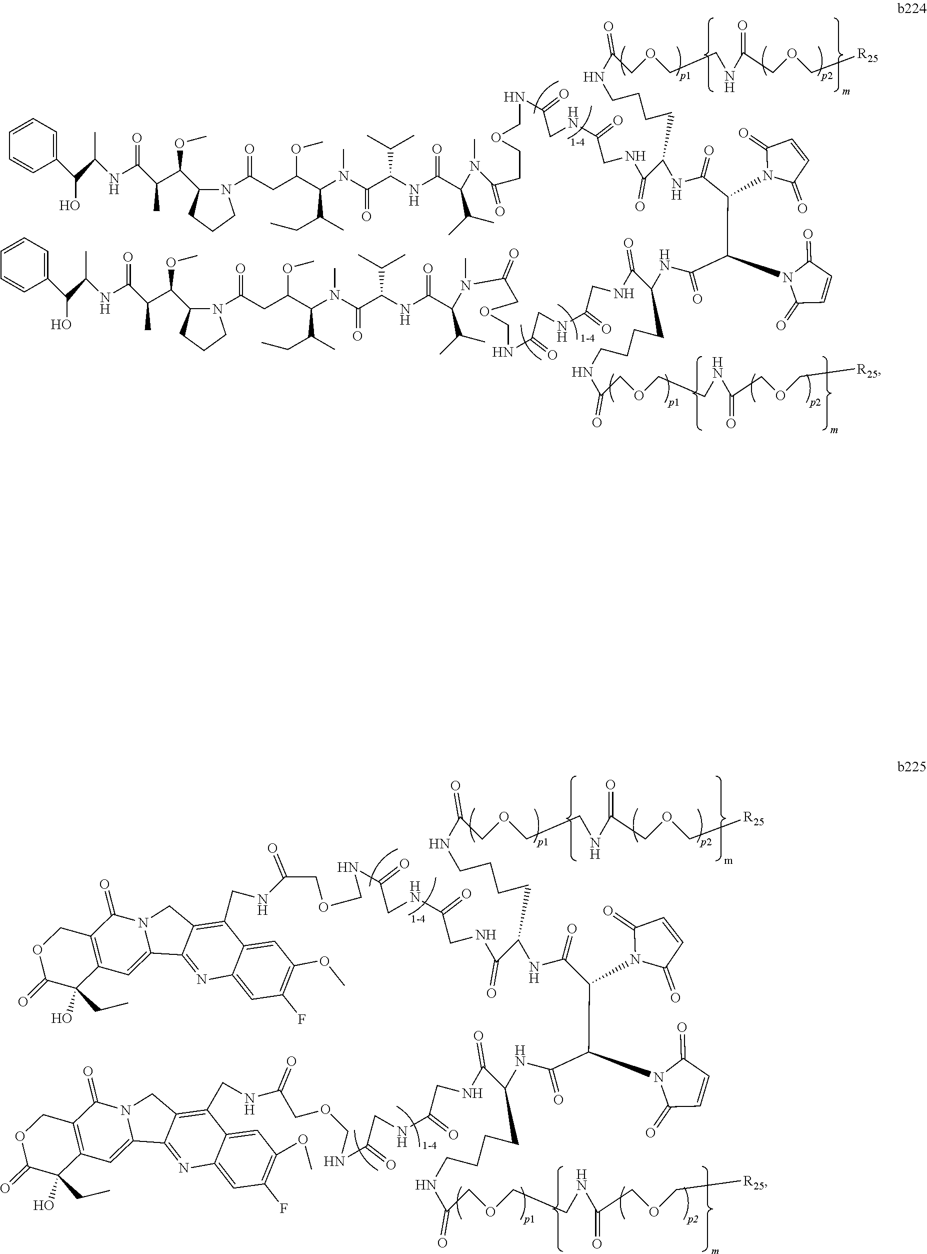 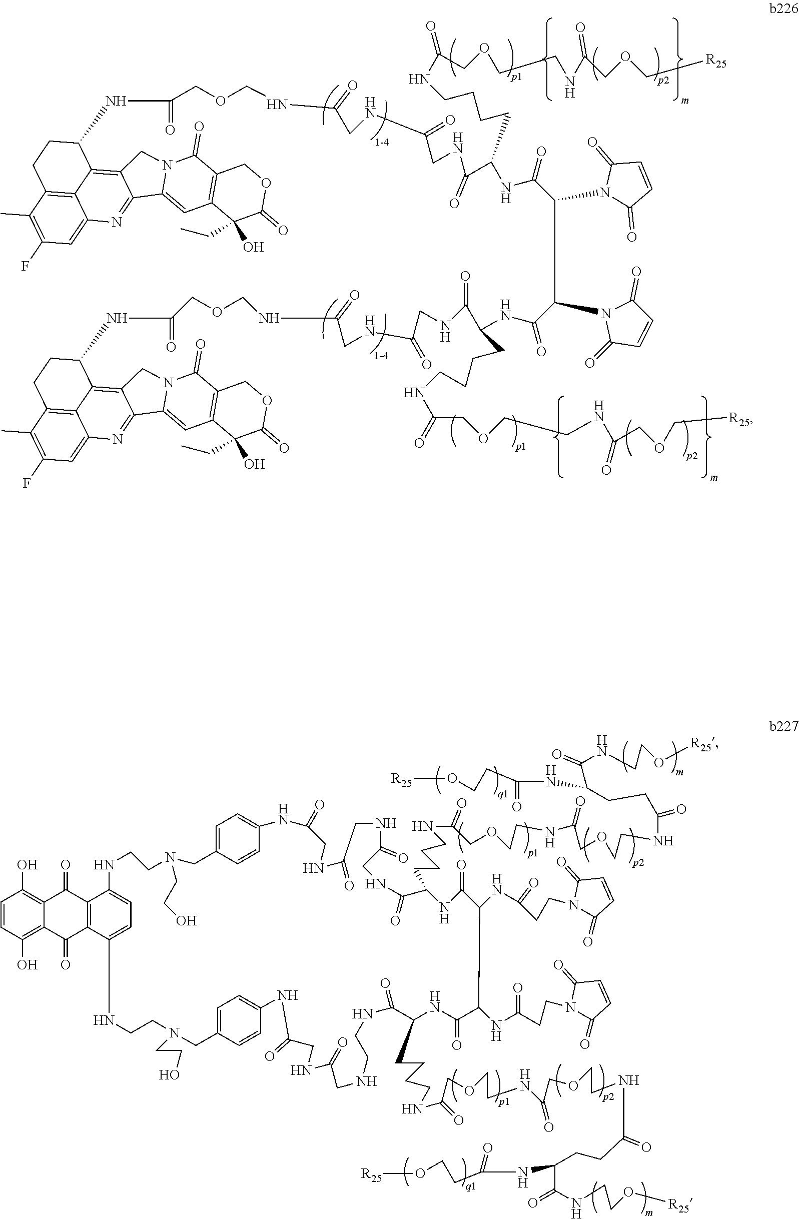 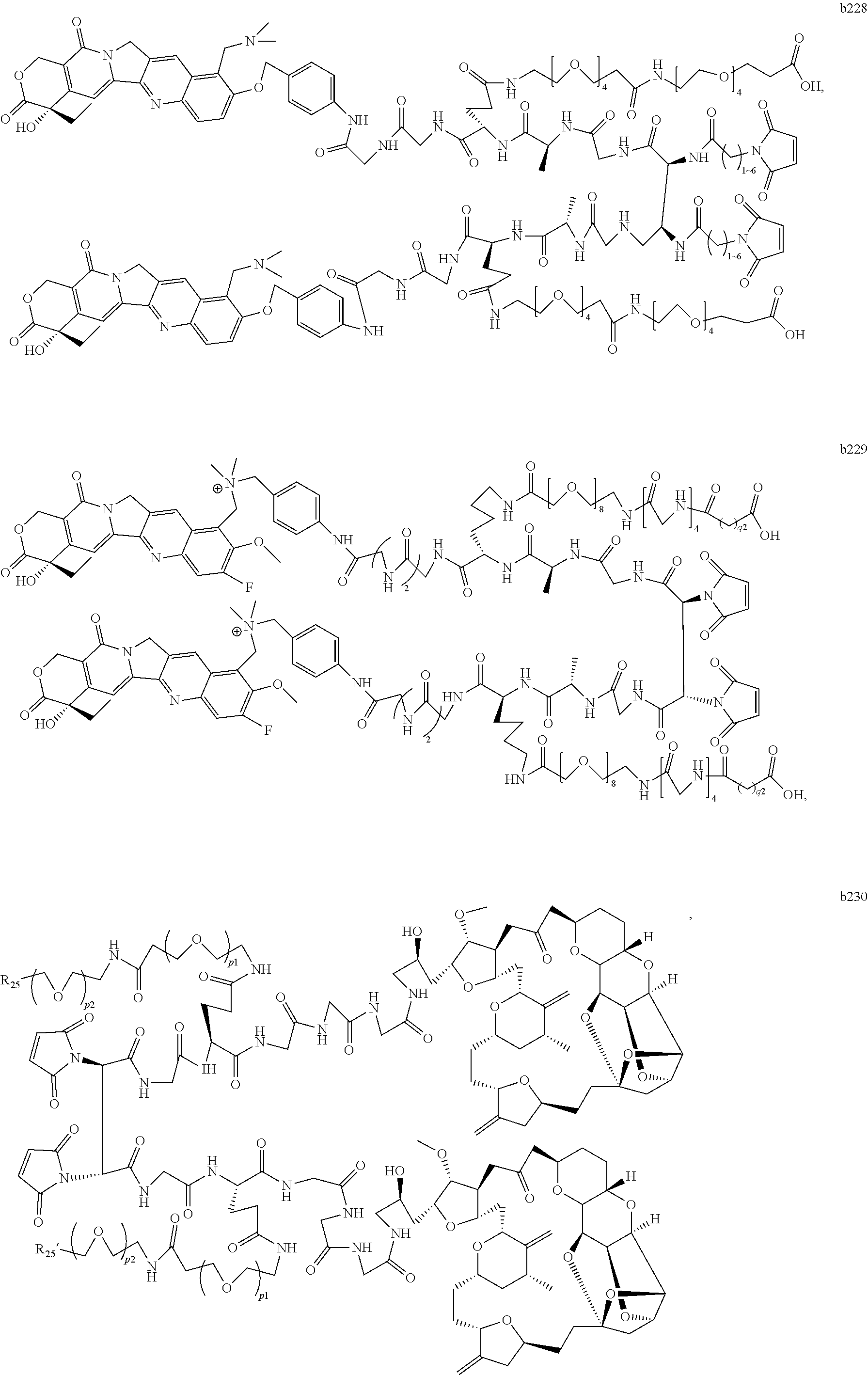 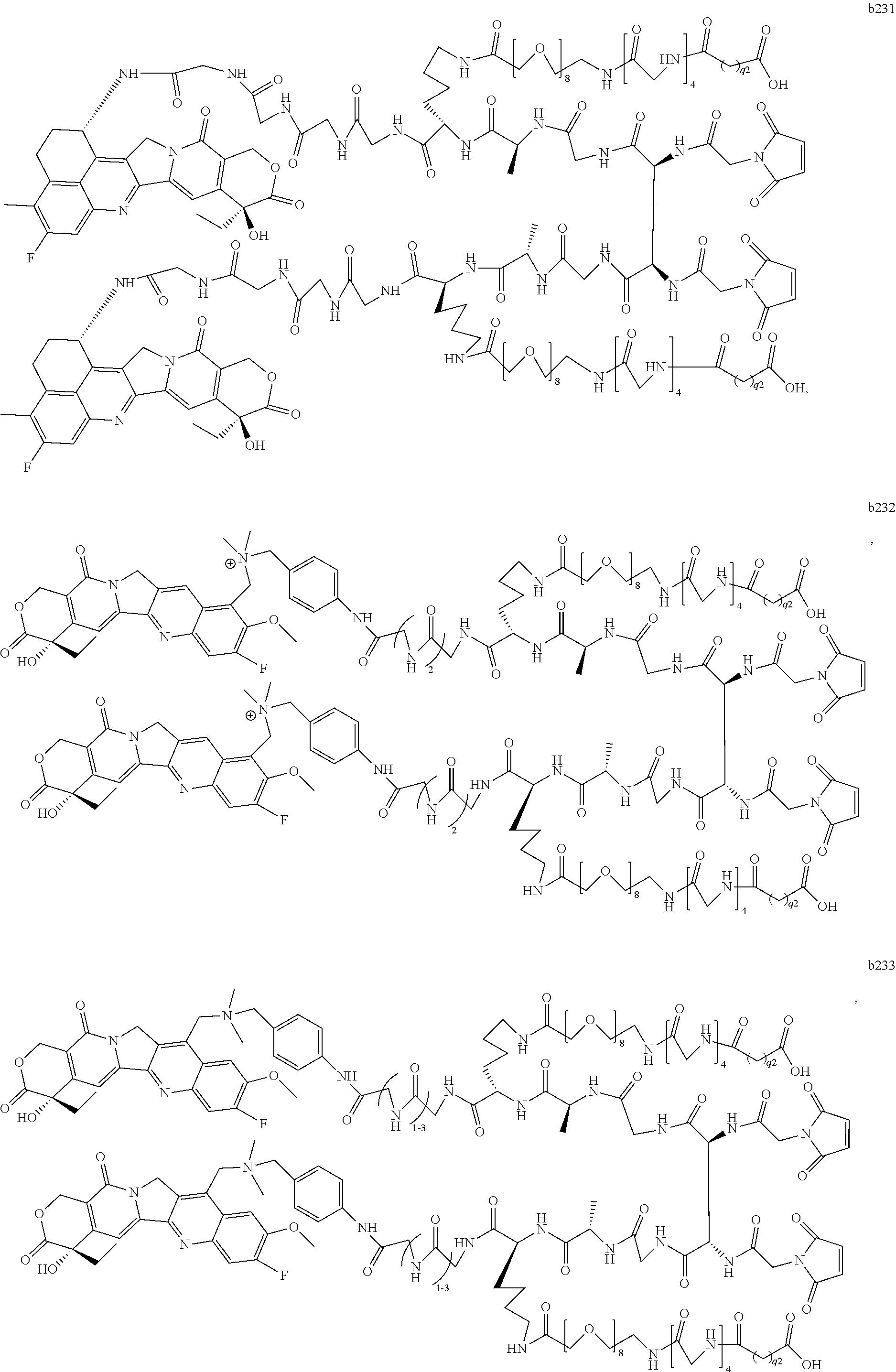 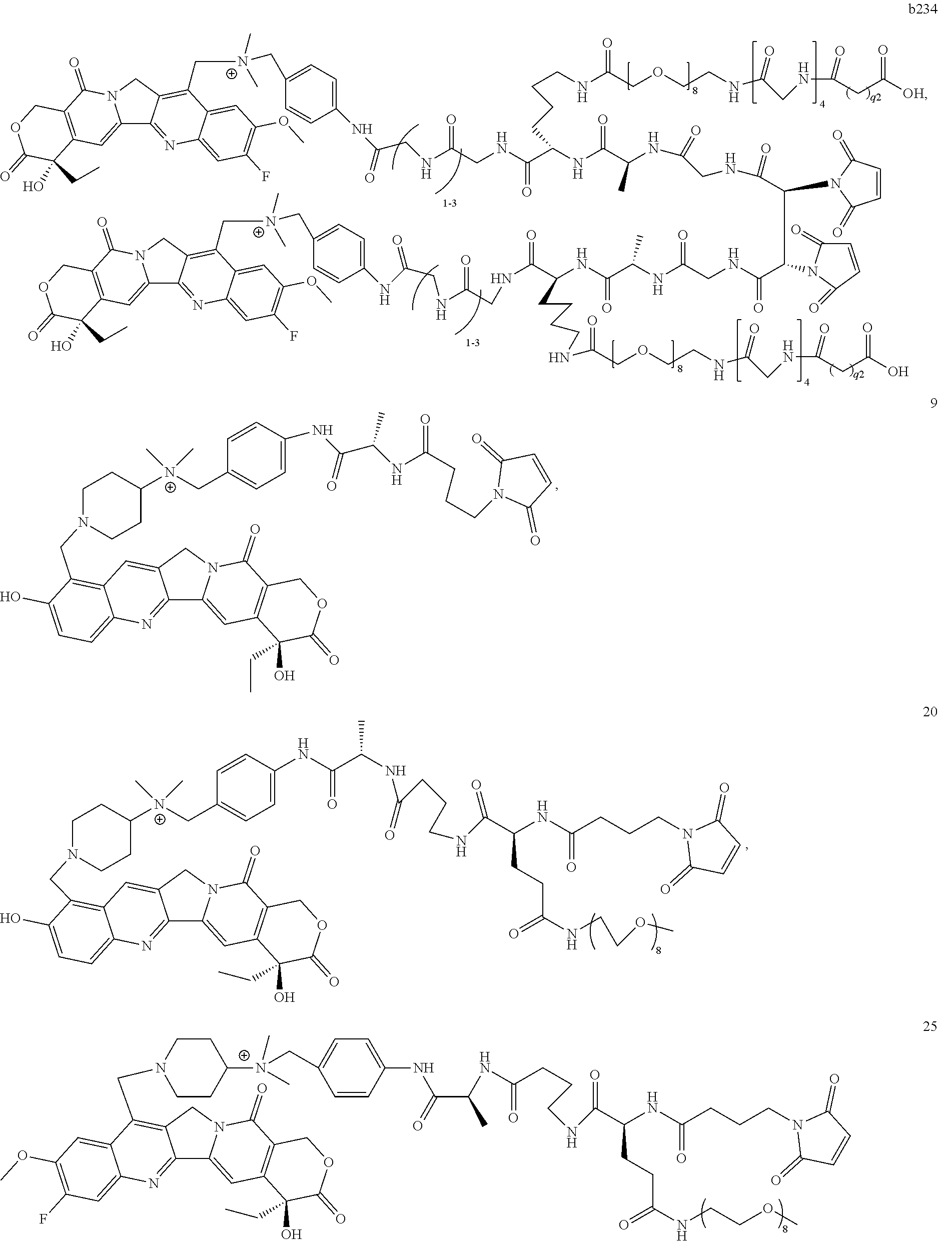 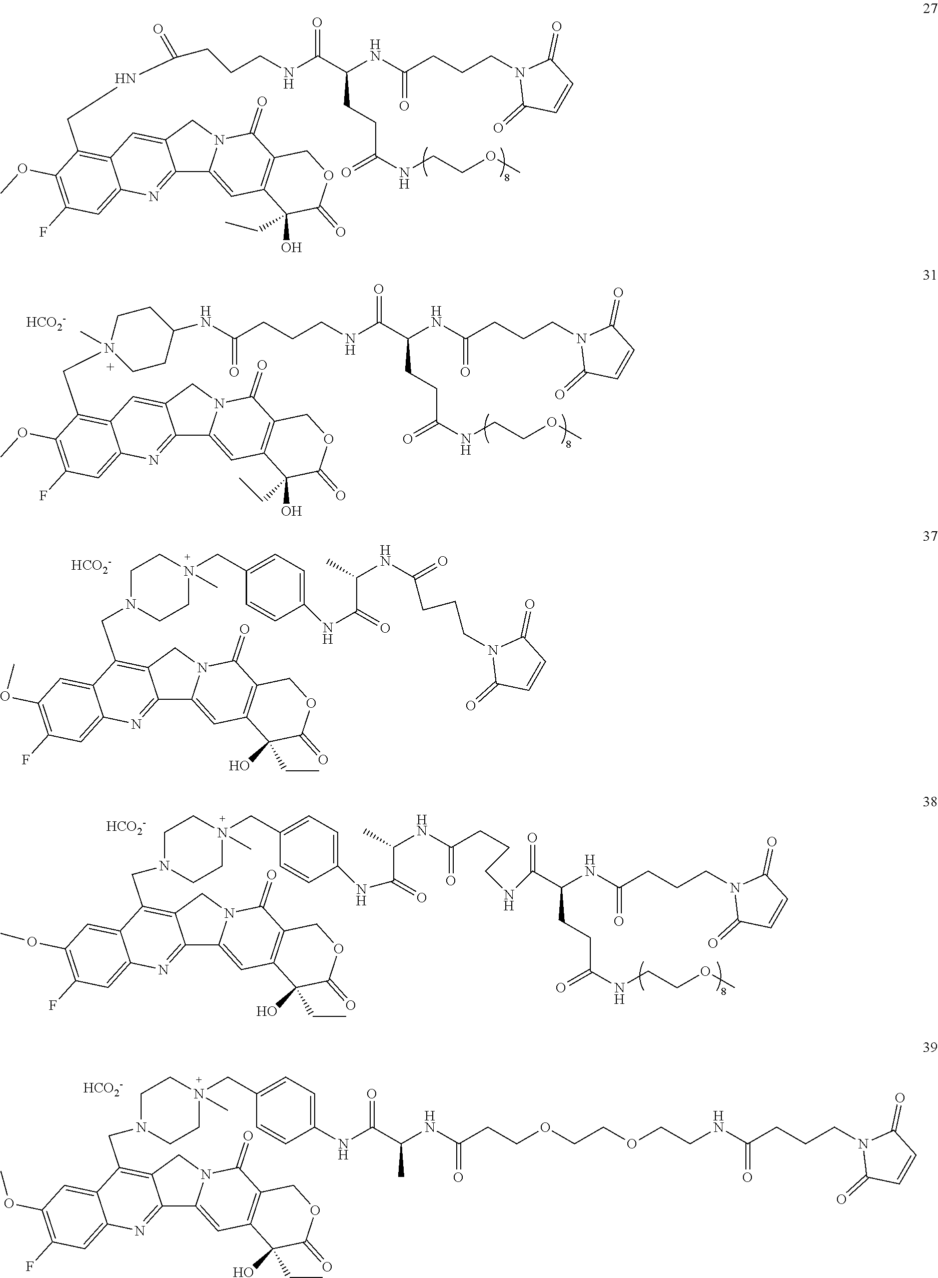 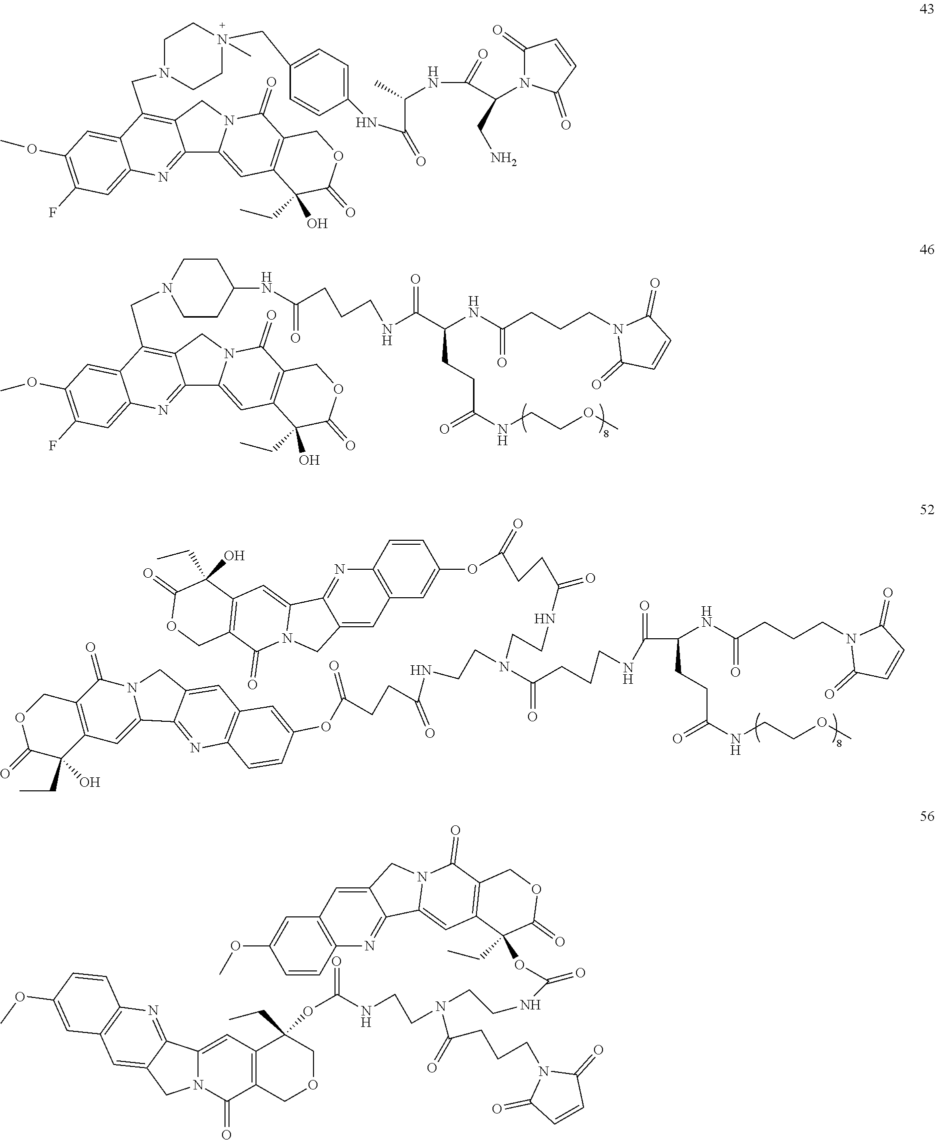 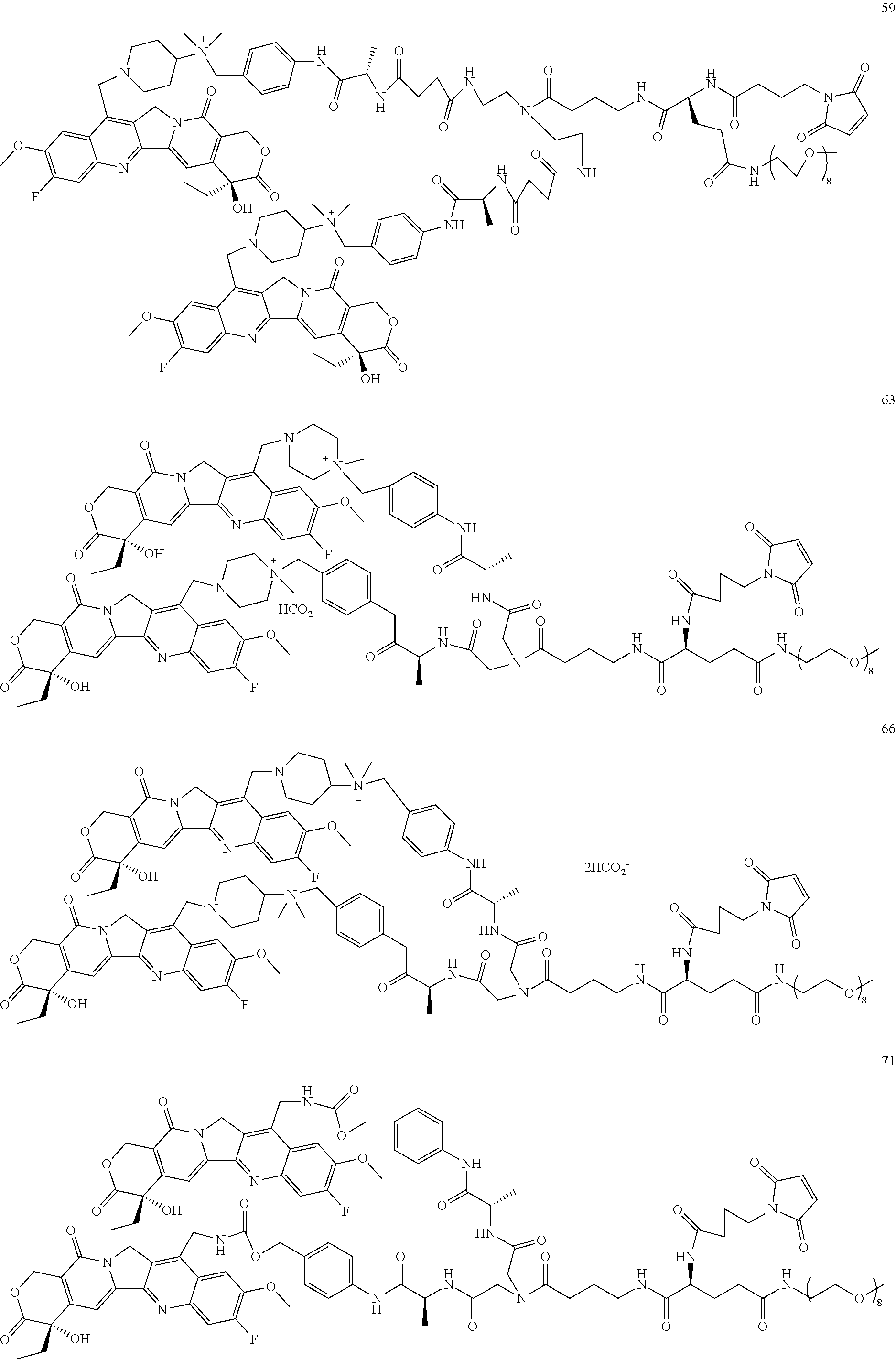 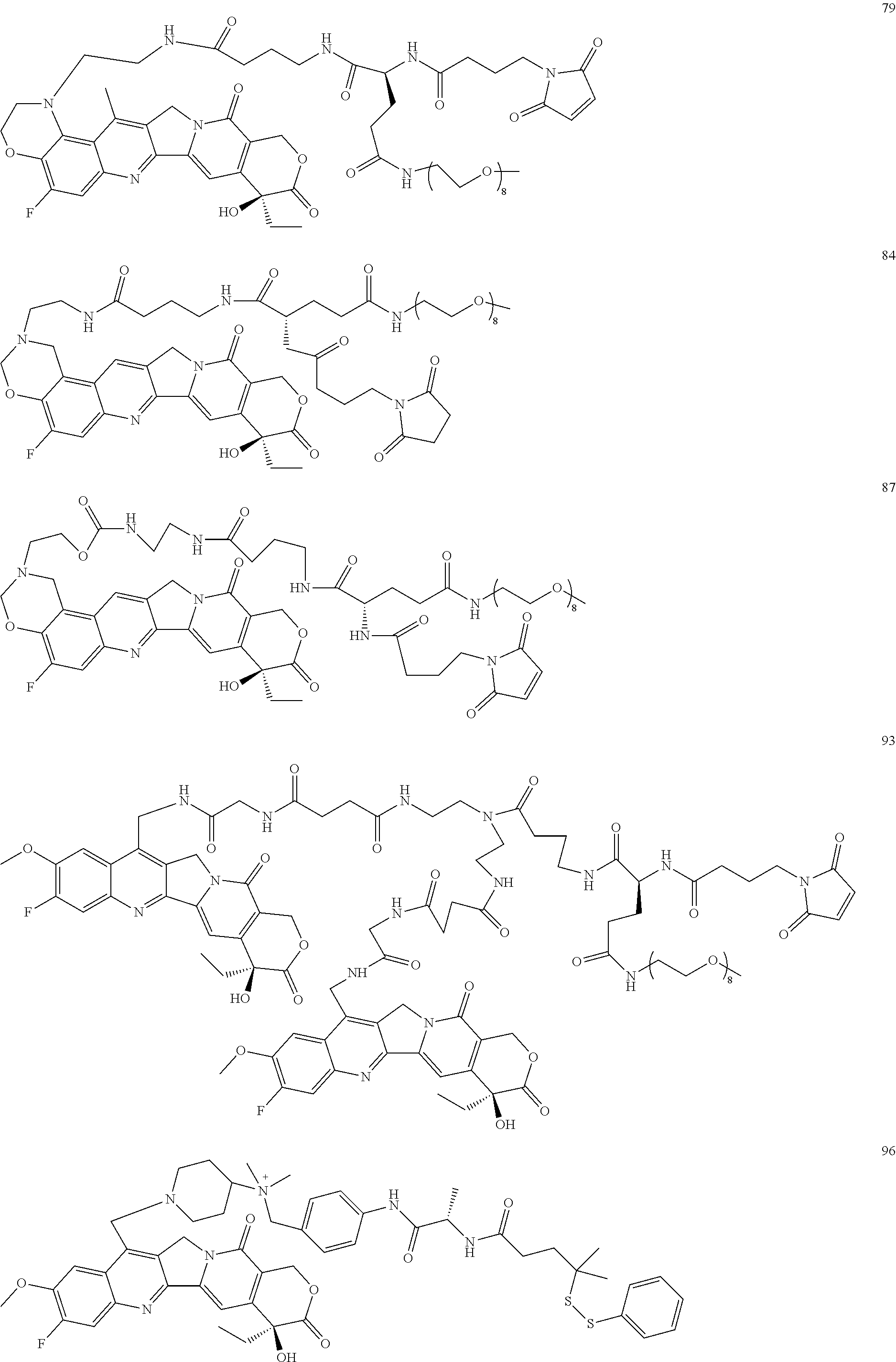 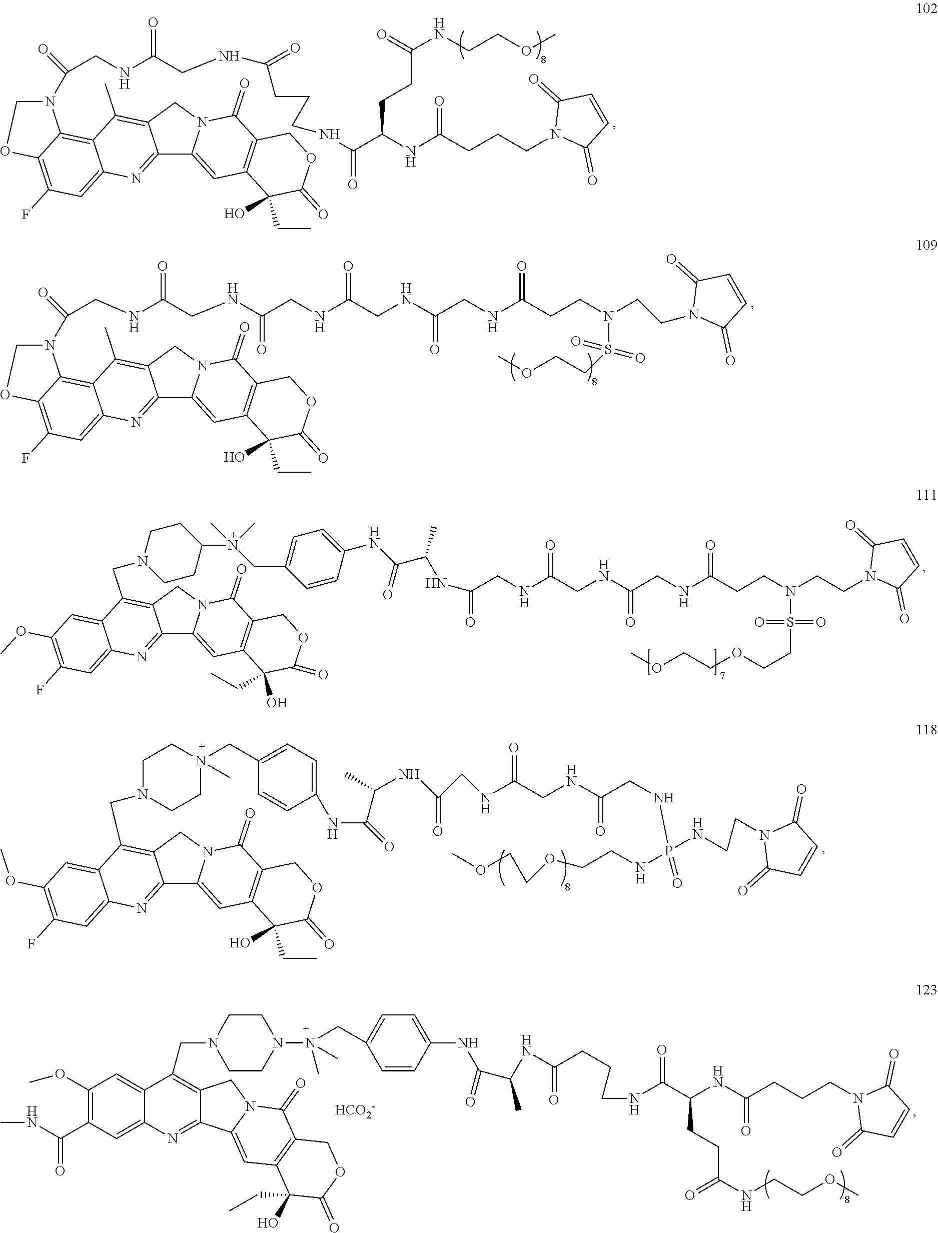 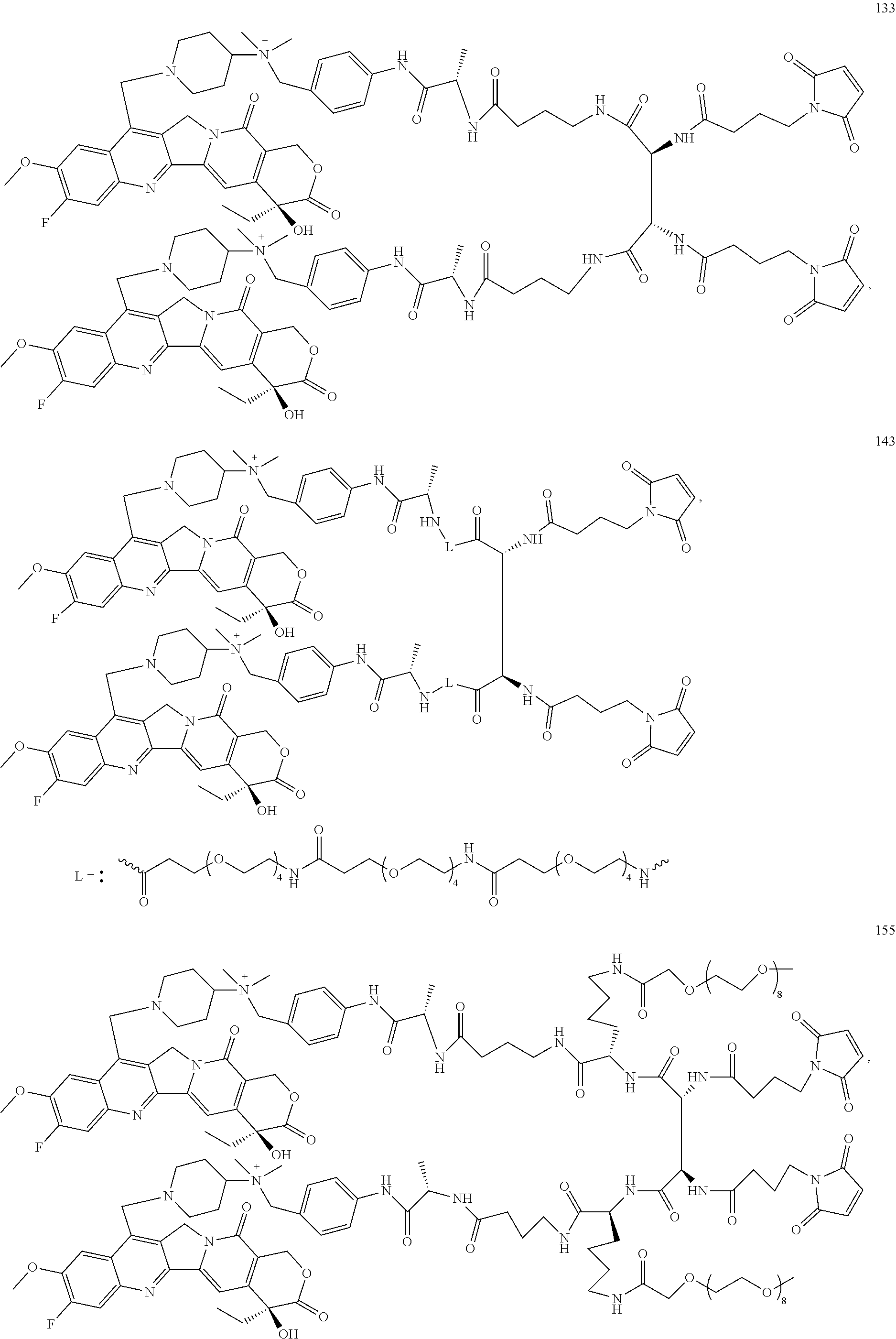 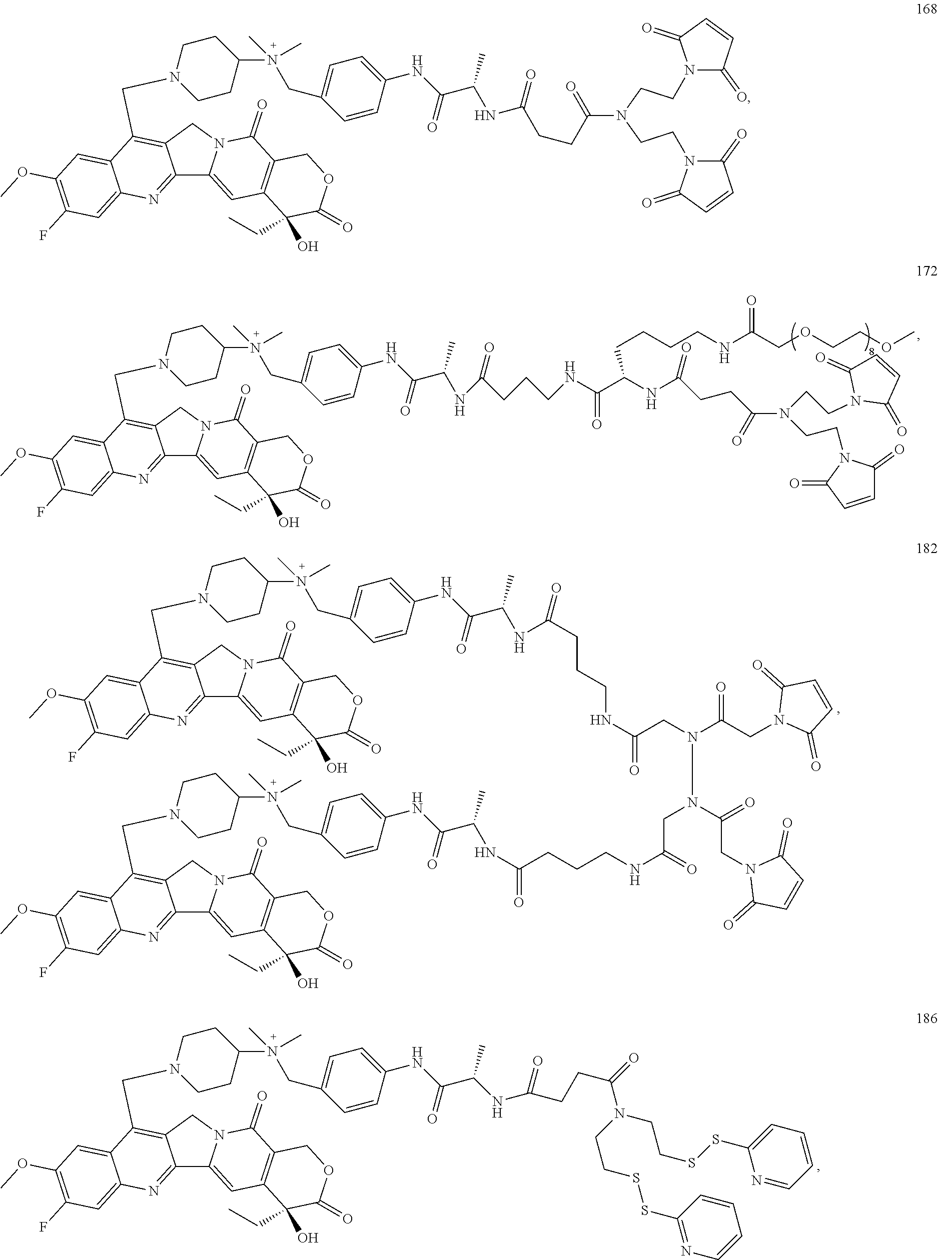 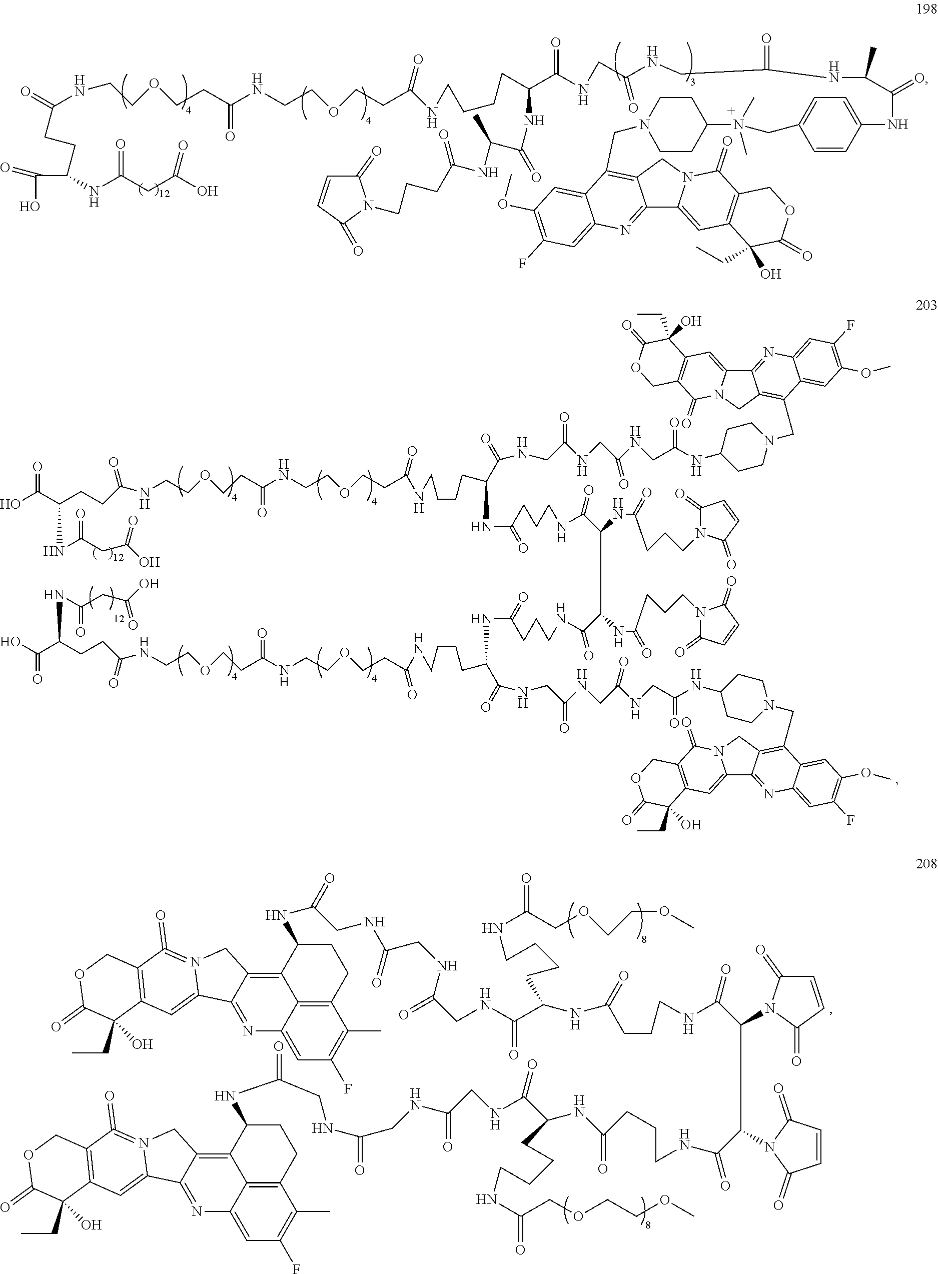 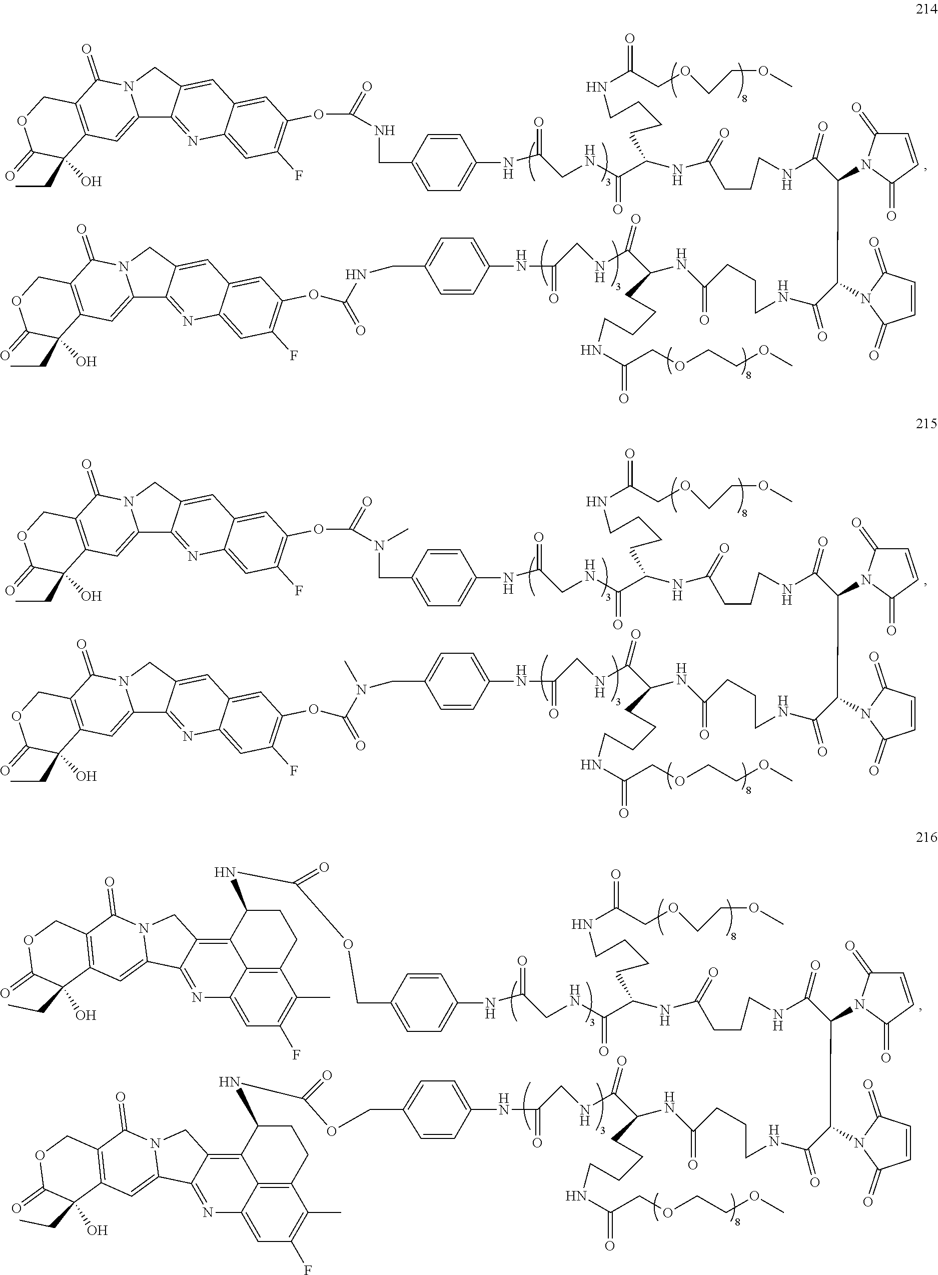 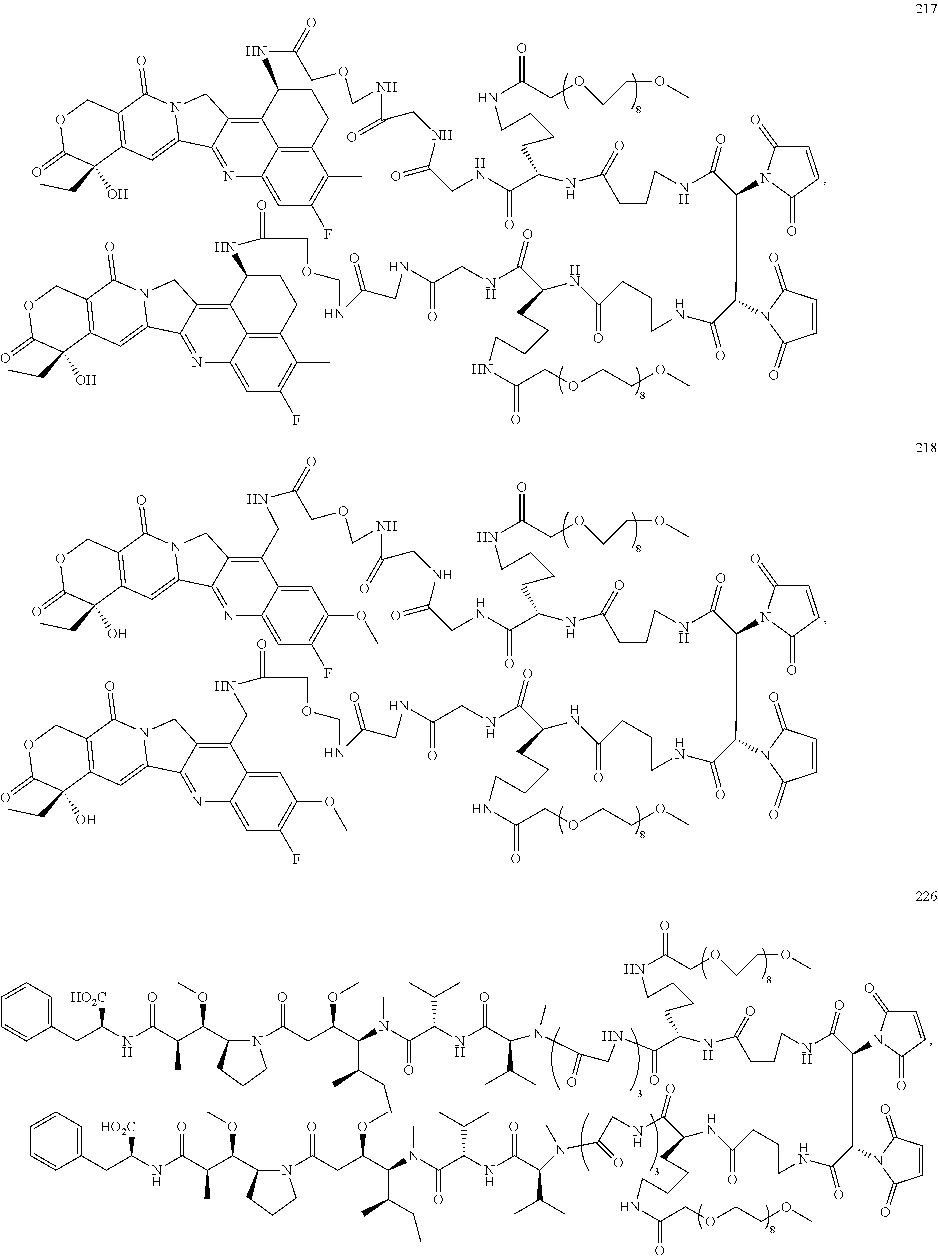 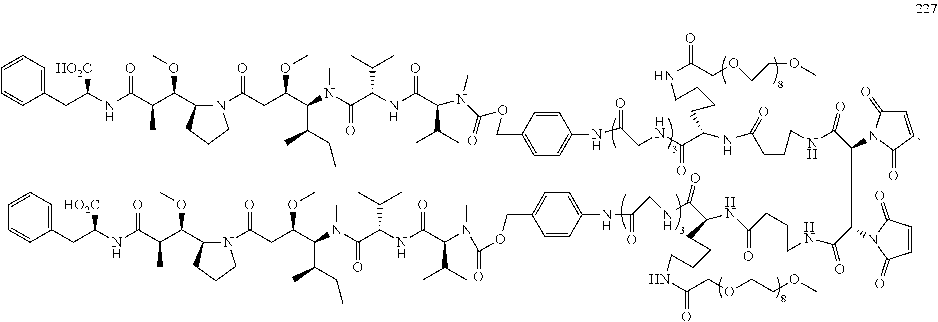 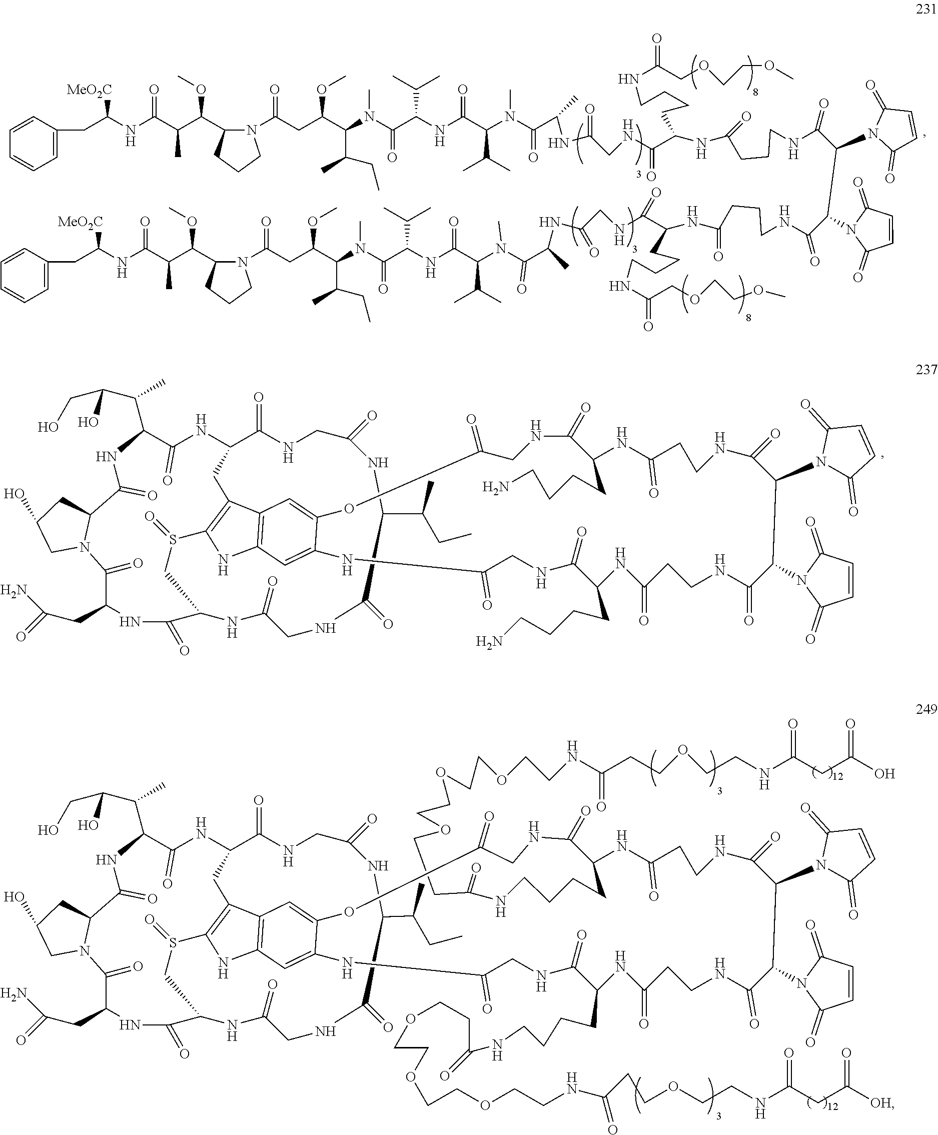 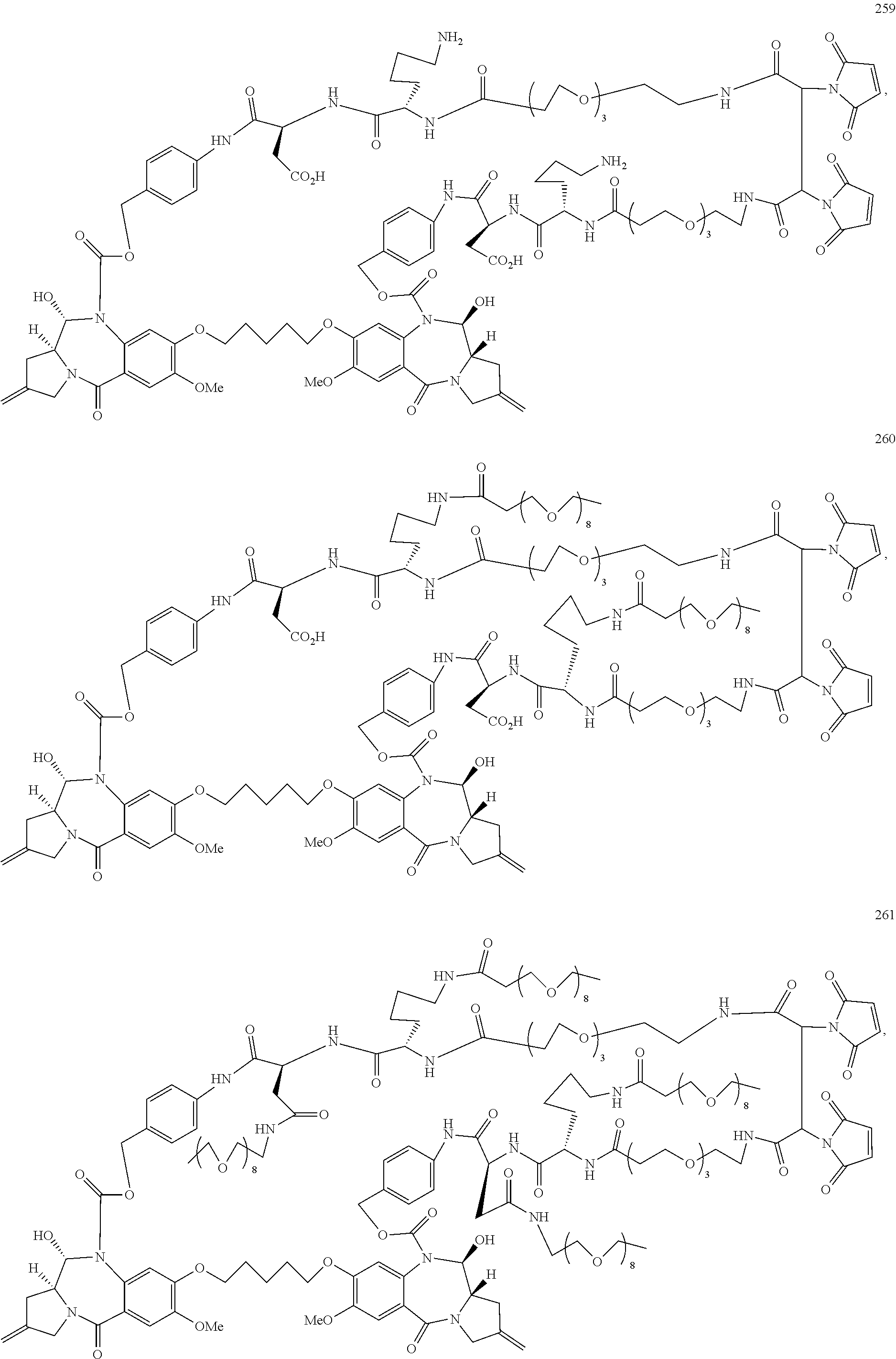 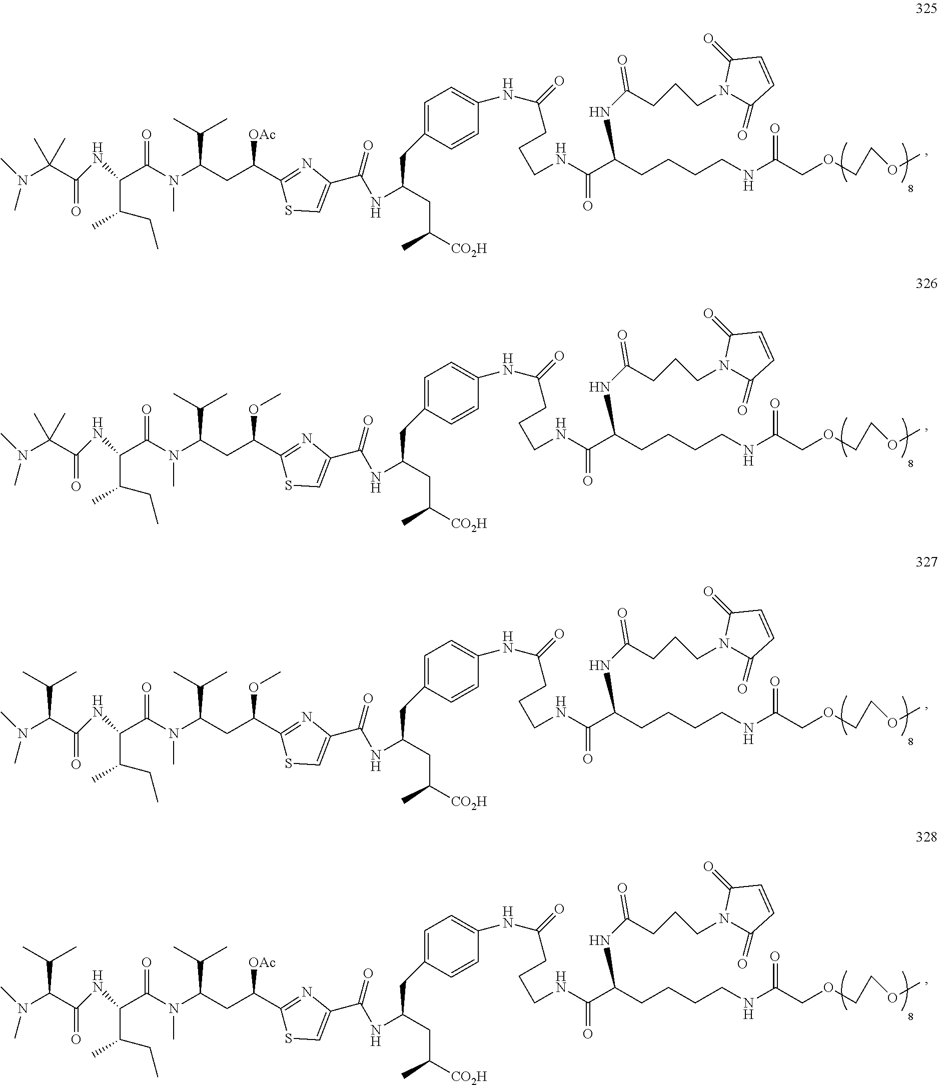 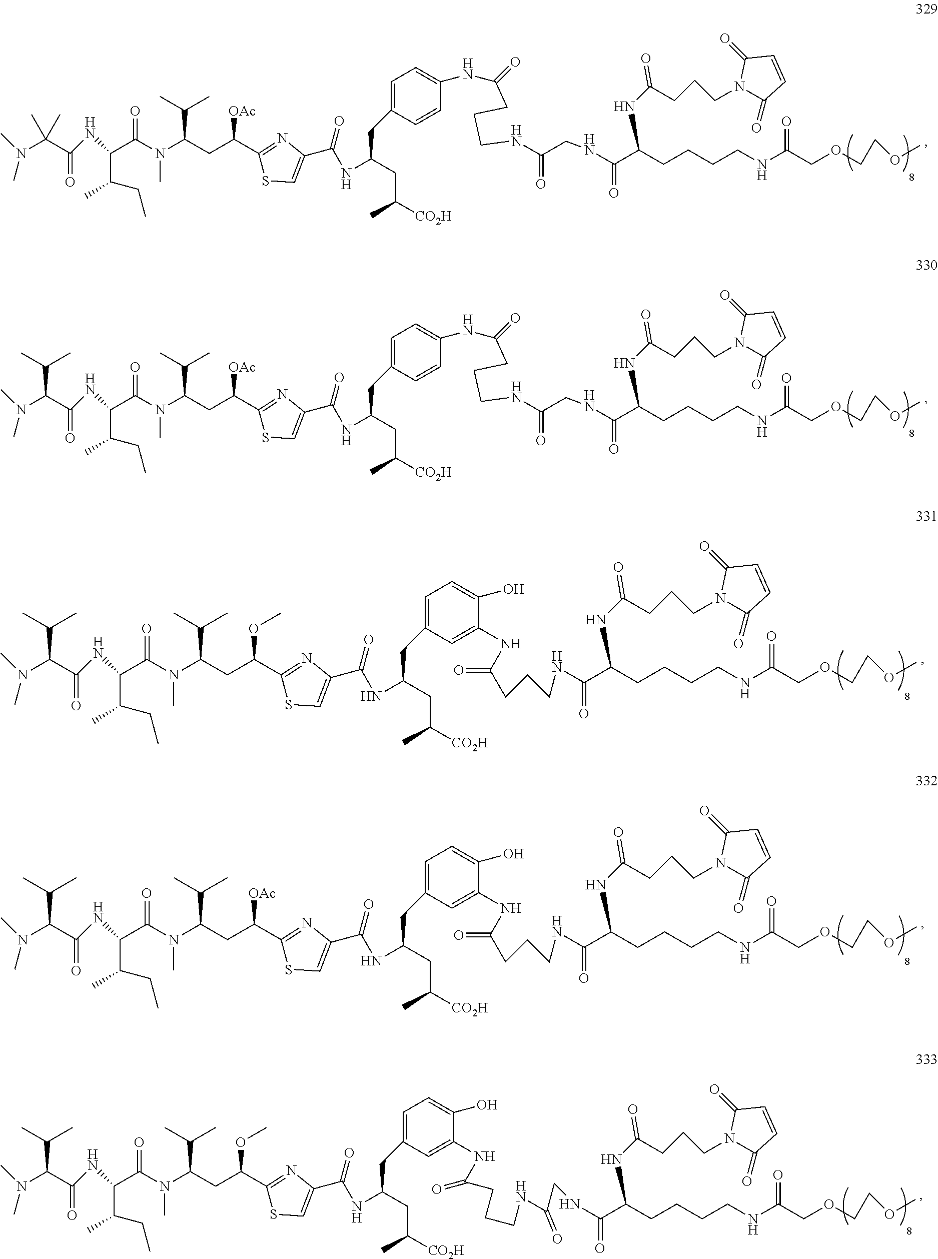 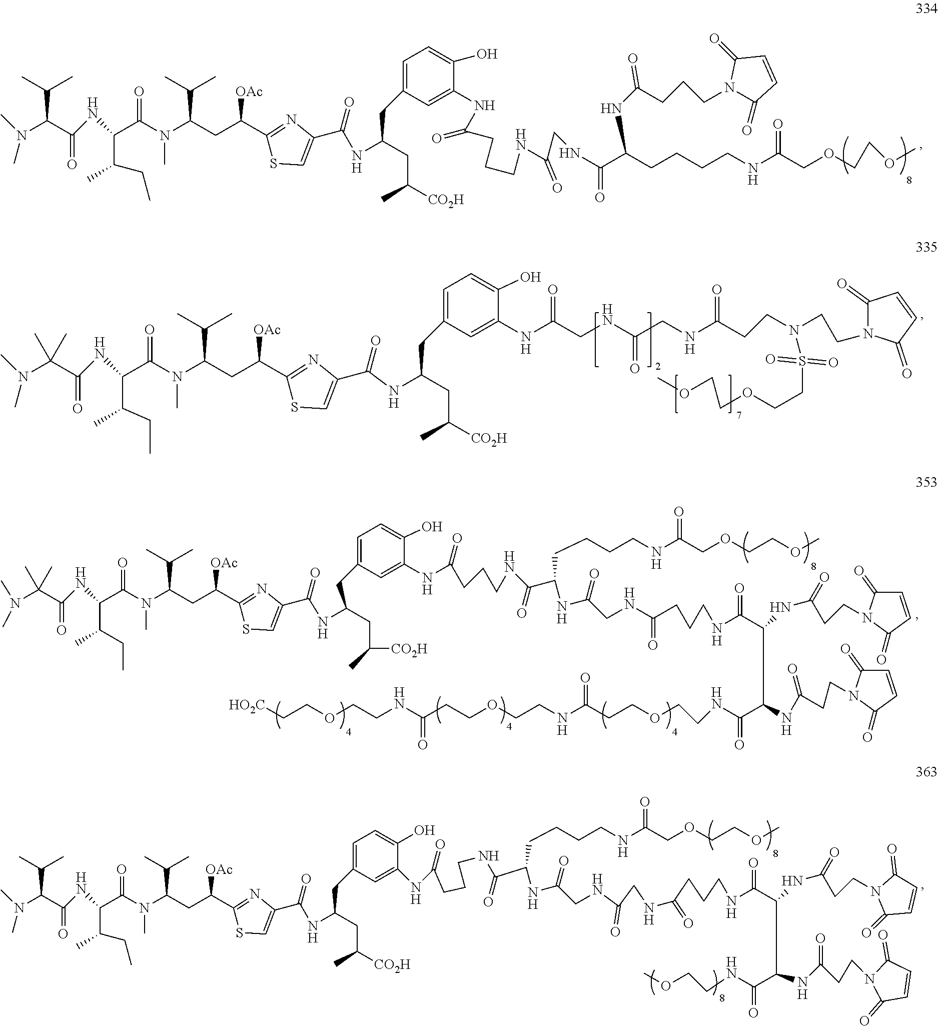 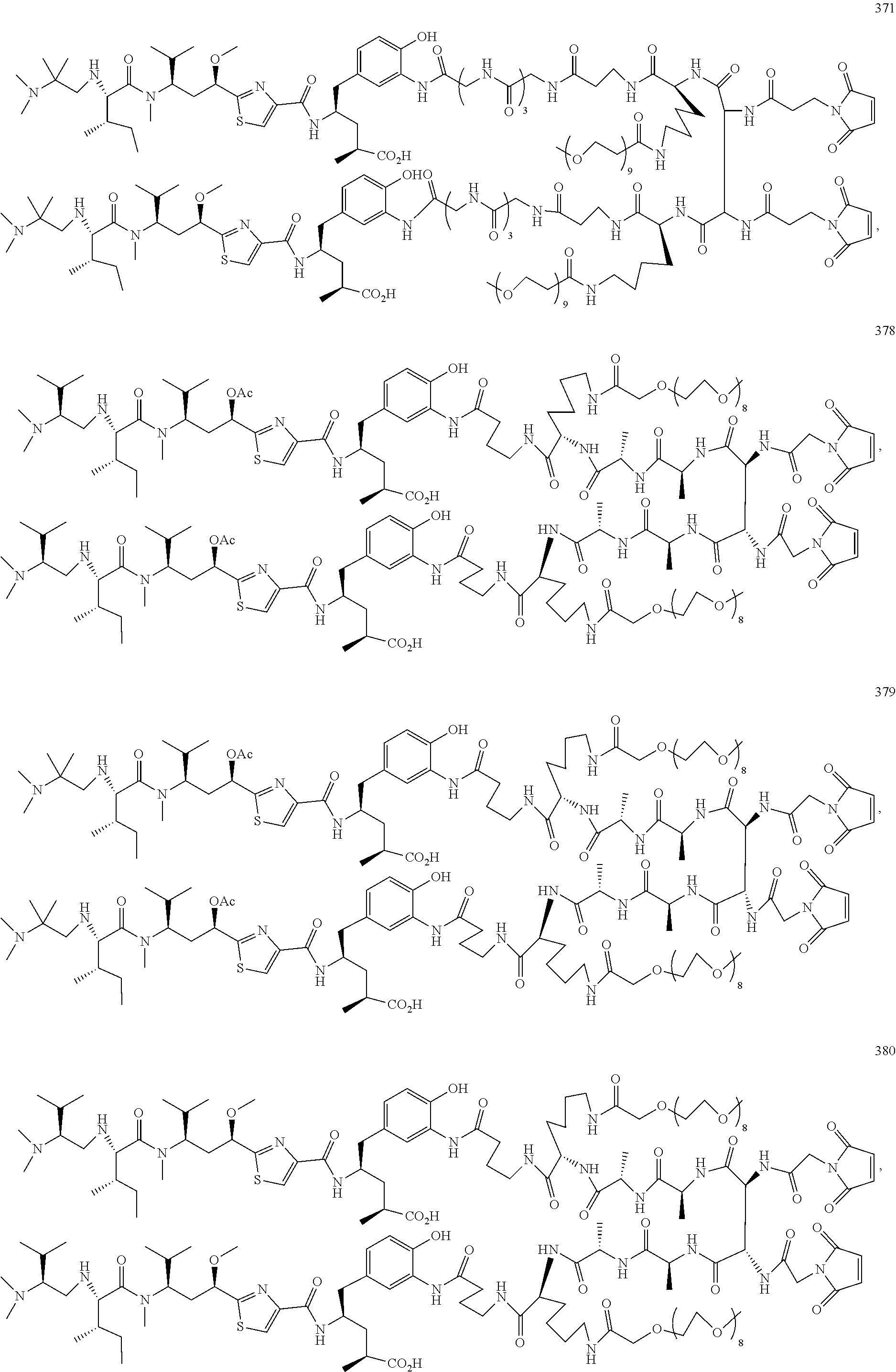 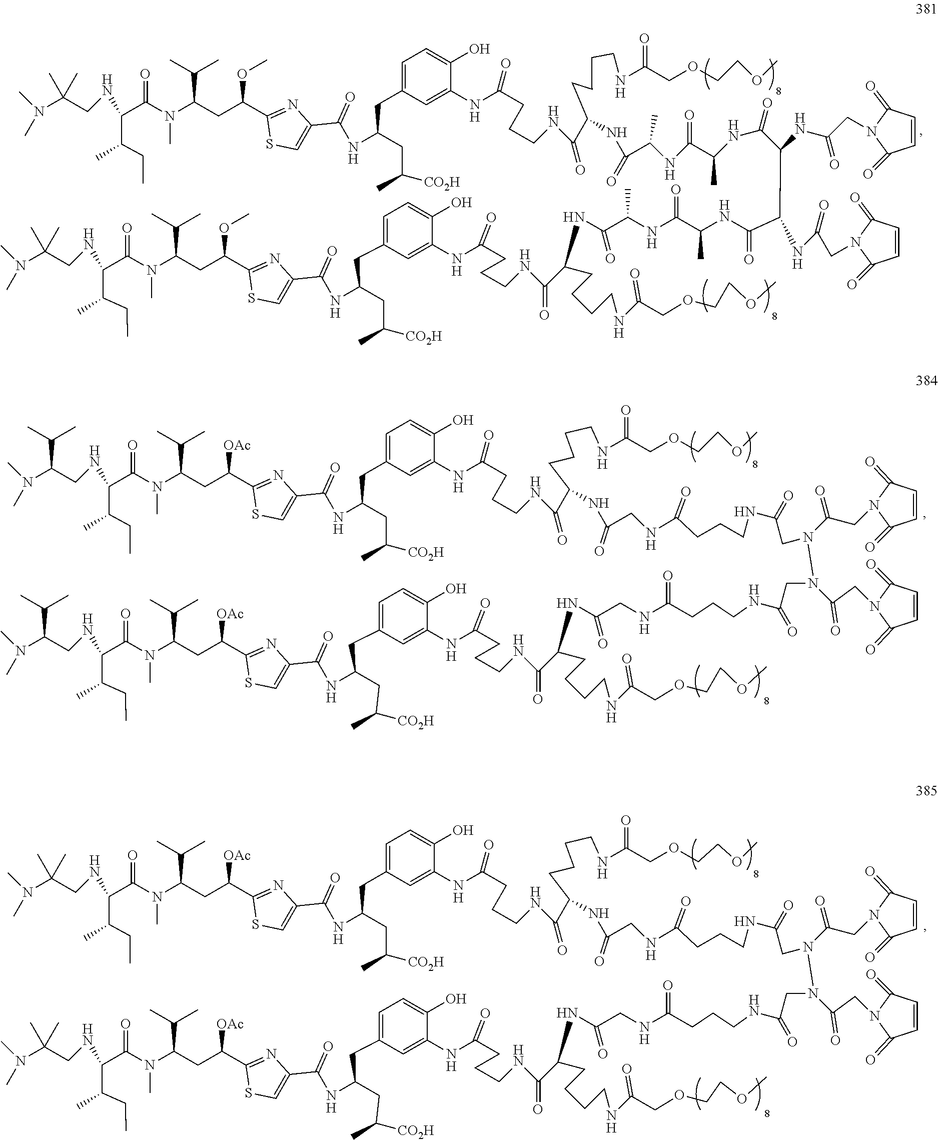 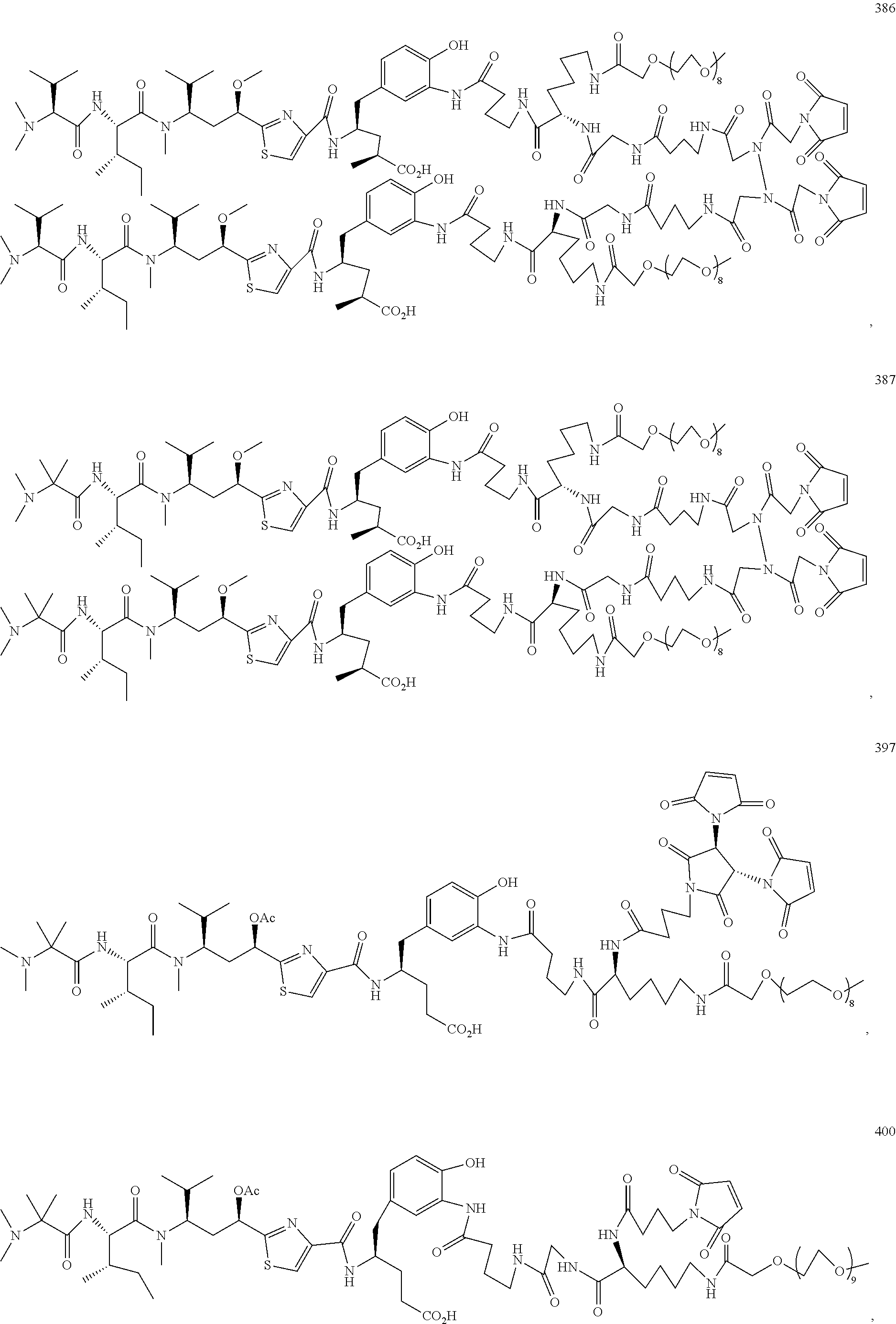 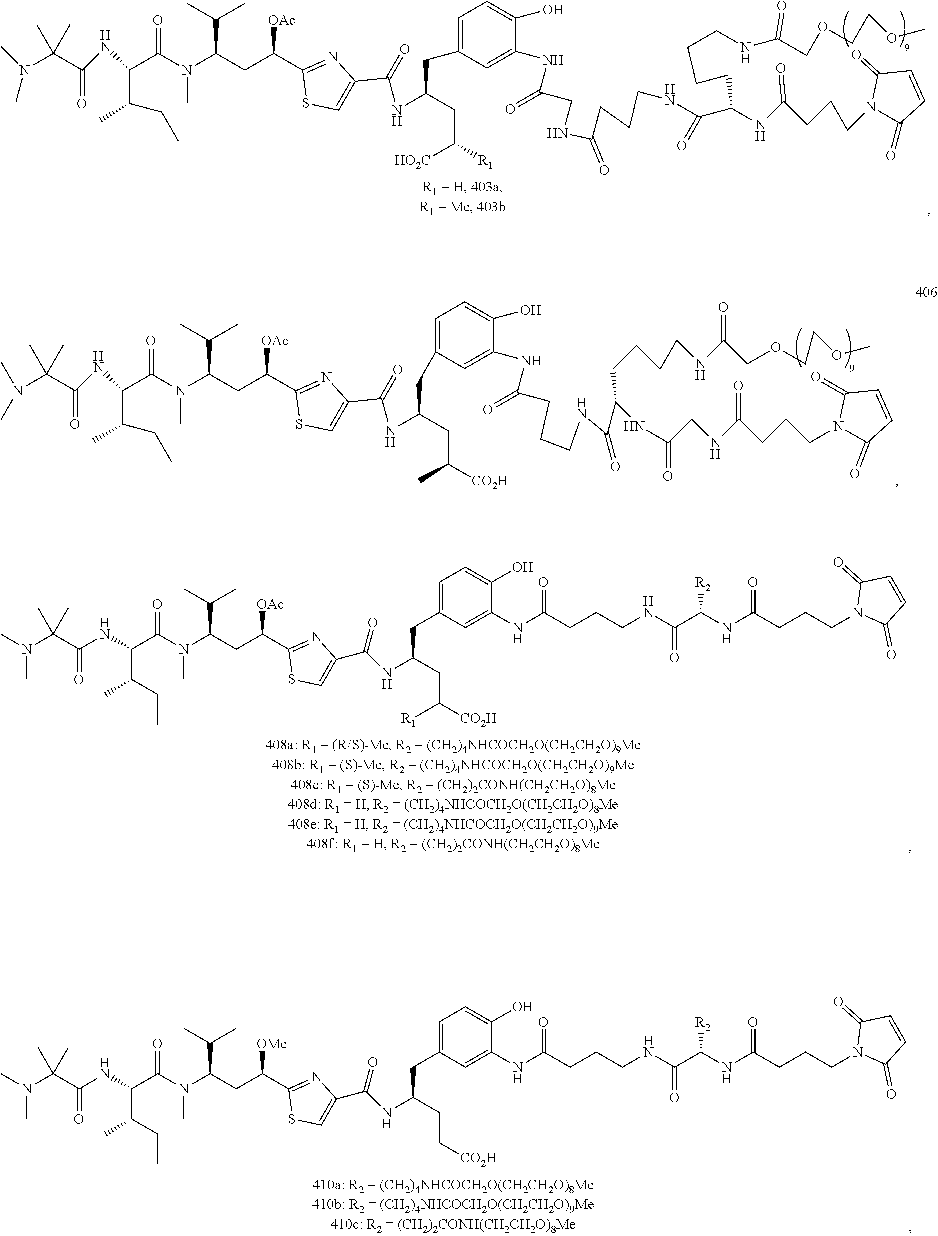 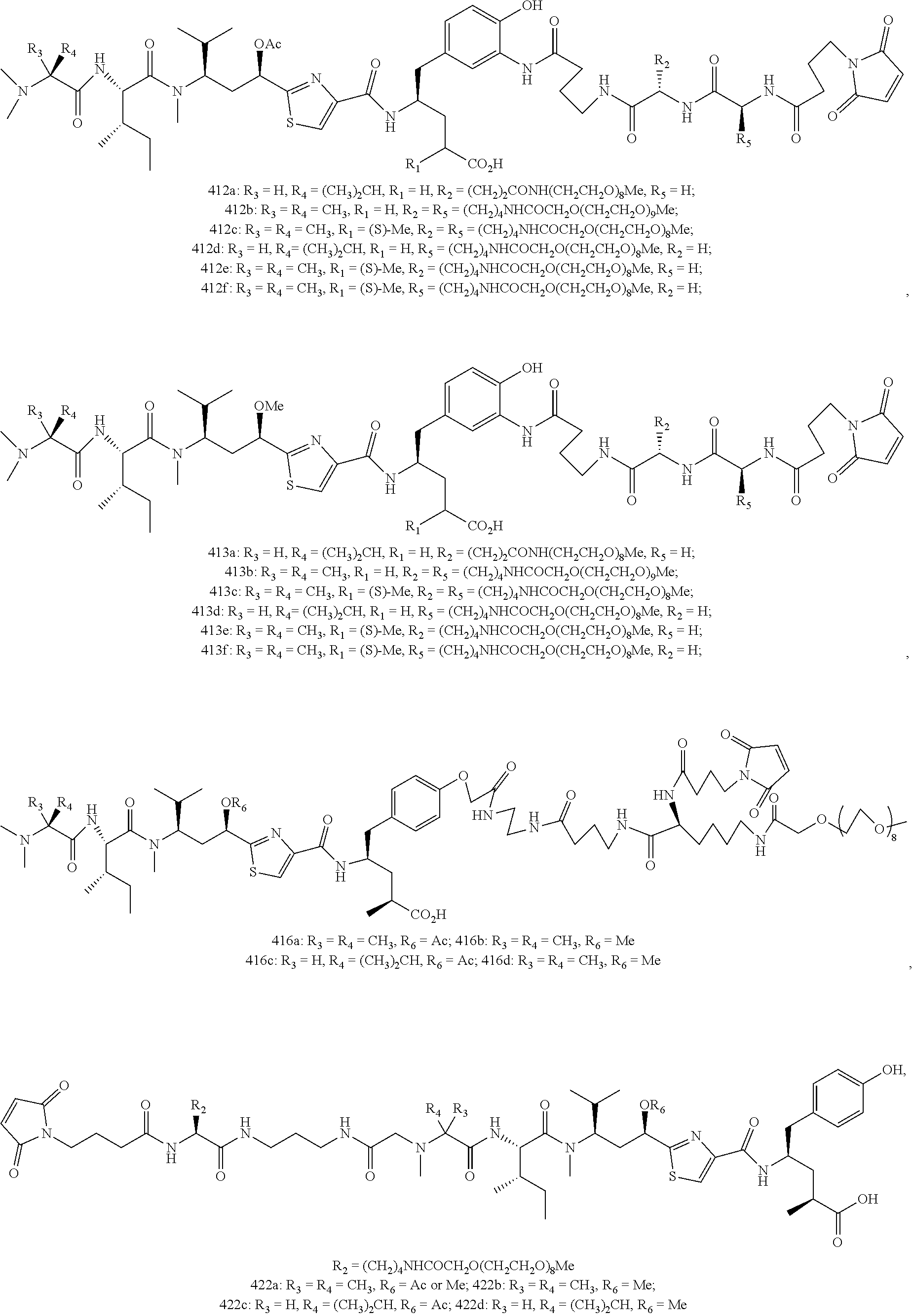 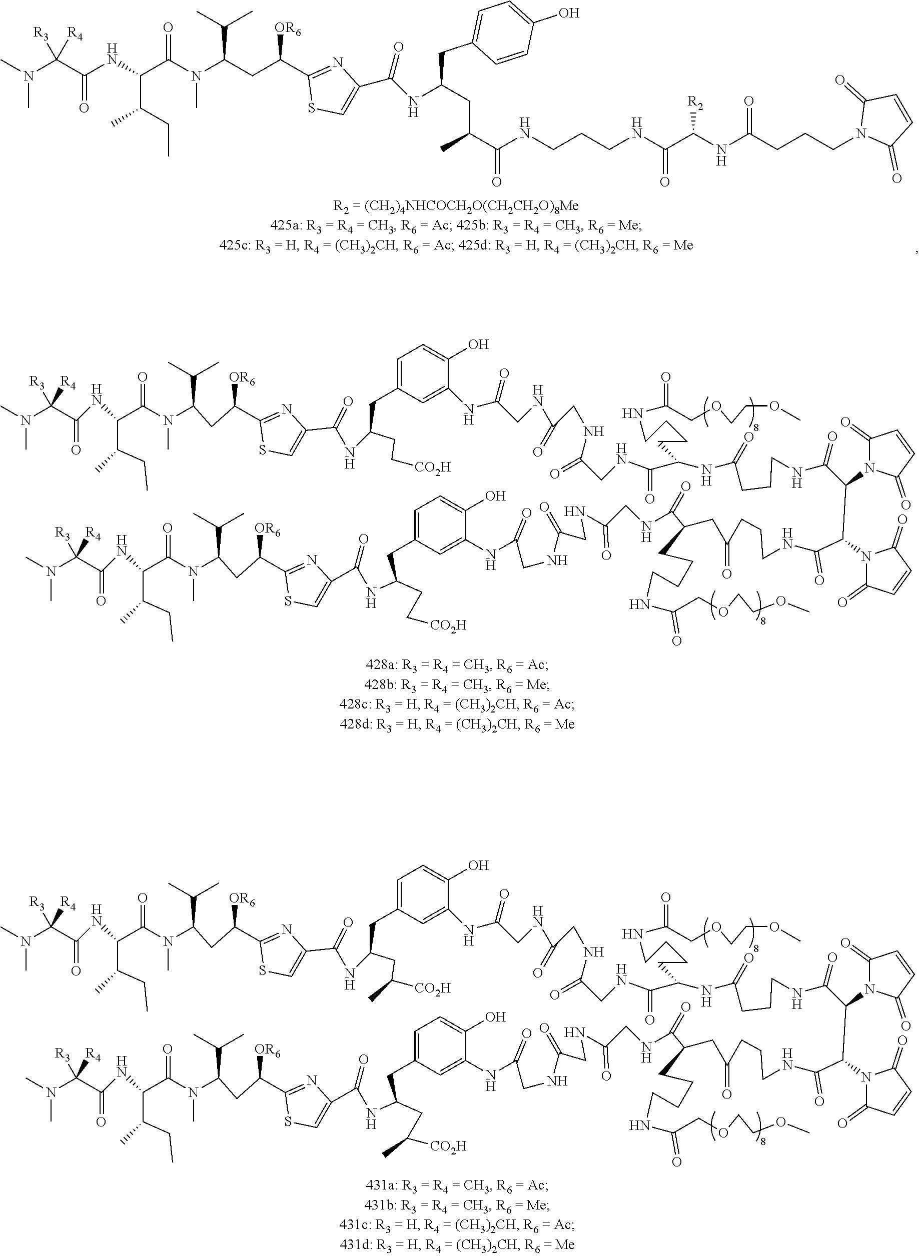
or a derivative with one or more isotopes, a pharmaceutically acceptable salt, hydrate, or hydrated salt thereof; or a polymorphic crystalline structure thereof; or an optical isomer, racemate, diastereomer or enantiomer thereof;
wherein m, m₁, m₂, n, P¹, R¹, R², R³, R⁴, R^{1′}, R^{2′}, R^{3′}, R₁, R₂, R₃, R₄, R₅, R₆, R₁₂, R₁₂′, R₂₅, R₂₆, R₂₅′, X₁, X₂, X₃, X₅, X₆, Y₁, Y₂, Y₆, Z₃, Z₅, p, p₁, p₂, p₃, q₁, q₂, Lv₁, Aa, (Aa)r, Ar and mAb are described the same as in claim 23.

37. The process according to claim 34, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

38. A pharmaceutical composition comprising a therapeutically effective amount of the conjugate of claim 43, and a pharmaceutically acceptable salt, carrier, diluent, or excipient therefor, or a combination of two or more of the conjugates, for treatment or prevention of a cancer, or an autoimmune disease, or an infectious disease.

39. The pharmaceutical composition according to claim 38, which is either in a liquid formula or in a formulated lyophilized solid/powder, comprising by weight of: 0.01%-99% of one or more of the conjugates; 0.0%-20.0% of one or more polyols; 0.0%-20.0% of one or more surfactants; 0.0%-5.0% of one or more preservatives; 0.0%-30% of one or more amino acids; 0.0%-5.0% of one or more antioxidants; 0.0%-0.3% of one or more metal chelating agents; 0.0%-30.0% of one or more buffer salts for adjusting pH of the composition to pH 4.5-7.5; and 0.0%-30.0% of one or more isotonic agents for adjusting osmotic pressure between from about 250 to 350 mOsm when being reconstituted for administration to a patient;
wherein the polyol is selected from the group consisting of fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and metallic salts thereof,
wherein the surfactant is selected from the group consisting of polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is selected from the group consisting of benzyl alcohol, octadecyldimethylbenzyl ammonium chloride, hexamethonium chloride, benzalkonium chloride, benzethonium chloride, phenol, butyl and benzyl alcohol, alkyl parabens, catechol, resorcinol, cyclohexanol, 3-pentanol, and m-cresol;
wherein the amino acid is selected from the group consisting of arginine, cystine, glycine, lysine, histidine, ornithine, isoleucine, leucine, alanine, glycine glutamic acid and aspartic acid;
wherein the antioxidant is selected from ascorbic acid, glutathione, cystine or and methionine;
wherein the chelating agent is EDTA or EGTA;
wherein the buffer salt is selected from the group consisting of sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; and arginine, glycine, glycylglycine, or histidine with anionic acetate, chloride, phosphate, sulfate, or succinate salt;
wherein the tonicity agent is selected from the group consisting of mannitol, sorbitol, sodium acetate, potassium chloride, sodium phosphate, potassium phosphate, trisodium citrate, and sodium chloride.

40. The pharmaceutical composition according to claim 38, which is packed in a vial, bottle, pre-filled syringe, or pre-filled auto-injector syringe, in a form of a liquid or lyophilized solid.

41. A method for synergistical treatment or prevention of a cancer, an autoimmune disease, or an infectious disease, comprising administering the pharmaceutical composition according to claim 38 concurrently with a synergistic agent of a chemotherapeutic agent, a radiation therapy agent, an immunotherapy agent, an autoimmune disorder agent, an anti-infectious agents or another conjugate.

42. The method according to claim 41, wherein the synergistic agent is one or more selected from the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-lb, Interferon alfa-2a, Interferon alfa-2b, Interferon beta, Interferon beta 1a, Interferon beta 1b, Interferon gamma-la, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L₁ antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and an analog, derivative, a pharmaceutically acceptable salt thereof, carrier, diluent or excipient thereof or a combination of two or more of the above compounds.

43. A conjugate of formula (V), (VI), or (VII): 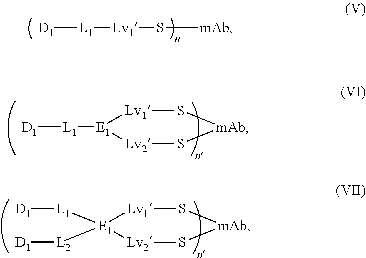
wherein n is 1-20; n′ is 1-10; mAb is an antibody or antibody-like protein;
wherein L₁ and L₂ are, the same or different, independently O, NH, S, NHNH, N(R₃), N(R₃)N(R₃′), polyethyleneoxy unit of formula (OCH₂CH₂)ₚOR₃, (OCH₂CH(CH₃))ₚOR₃, NH(CH₂CH₂O)ₚR₃, or NH(CH₂CH(CH₃)O)ₚR₃, N[(CH₂CH₂O)ₚR₃][(CH₂CH₂O)ₚ′R₃′], (OCH₂CH₂)ₚCOOR₃, CH₂CH₂(OCH₂CH₂)ₚCOOR₃, wherein p and p′ are an integer independently selected from 0 to about 1000; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; wherein R₃ and R₃′ are independently H; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or 1-8 natural or unnatural amino acids; or polyethyleneoxy unit of formula (OCH₂CH₂)ₚ or (OCH₂CH(CH₃))ₚ, wherein p is an integer from 0 to about 1000, or a combination of two or more of the above;
wherein L₁ or L₂ may contain a self-immolative or a non-self-immolative component, peptidyl unit, a hydrazone bond, a disulfide, an ester, an oxime, an amide, or a thioether bond, the self-immolative unit is an aromatic compound that is electronically similar to a para-aminobenzylcarbamoyl (PAB) group, and is selected from the group consisting of 2-aminoimidazol-5-methanol compounds, heterocyclic PAB compounds, beta-glucuronide, and ortho or para-aminobenzylacetals;
wherein the self-immolative linker component has one of the following structures: 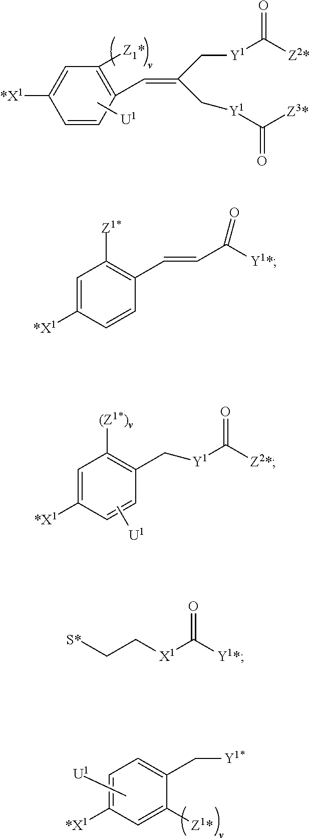
wherein a (*) atom is a point of attachment of additional spacer or releasable linker unit, or a cytotoxic agent, and/or a cell binding molecule (CBA); X¹, Y¹, Z² and Z³ are independently NH, O, or S; Z¹ is independently H, NH, O or S; v is O or 1; U¹ is independently H, OH, C₁-C₆ alkyl, (OCH₂CH₂)ₙF, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′) wherein R₅ and R₅′ are as defined above;
wherein the non-self-immolative linker component is one of the following structures: 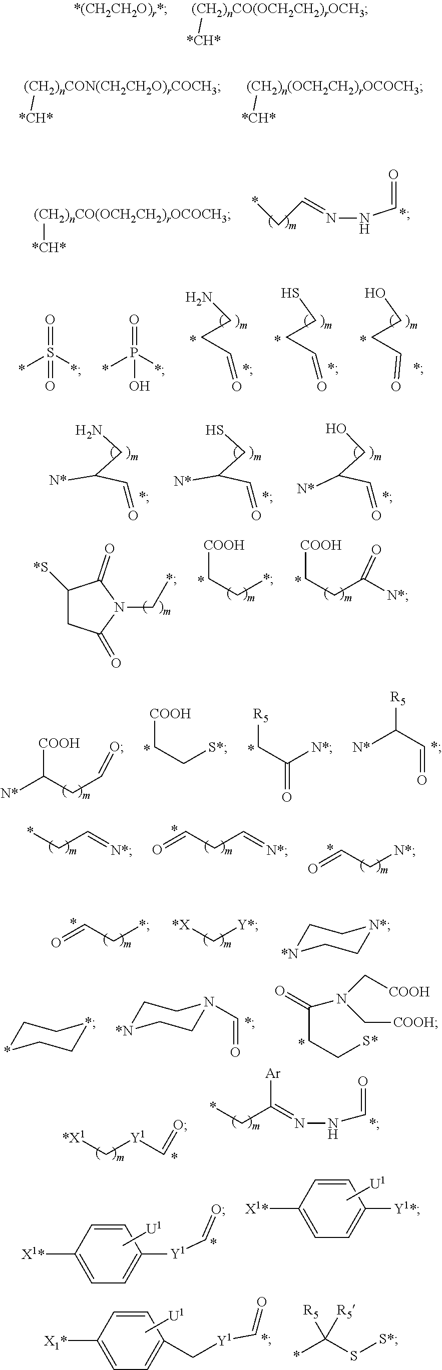 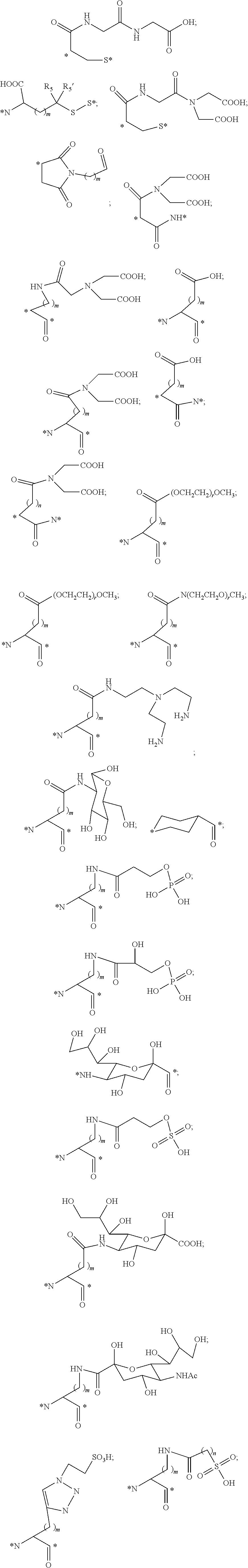 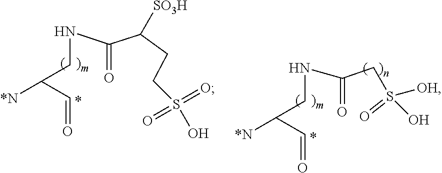
wherein a (*) atom is a point of attachment of additional spacer R₁ or releasable linker, cytotoxic agent, and/or binding molecule; X¹, Y¹, U¹, R₁, R₅, R₅′ are defined as above; r is 0-100; m and n are 0-6 independently;
wherein L₁ or L₂ may be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or a natural or unnatural peptide having 1-8 natural or unnatural amino acid units;
wherein L₁ or L₂ may be a releasable linker;
wherein the releasable linker (L₁ or L₂) comprises:
—(CR₅R₆)ₘ(Aa)r(CR₇R₈)ₙ(OCH₂CH₂)ₜ—, —(CR₅R₆)ₘ(CR₇R₈)ₙ(Aa)r(OCH₂CH₂)ₜ—, -(Aa)ᵣ- (CR₅R₆)ₘ(CR₇R₈)ₙ(OCH₂CH₂)ₜ—, —(CR₅R₆)ₘ(CR₇R₈)ₙ(OCH₂CH₂)ᵣ(Aa)-, —(CR₅R₆)ₘ—(CR₇═CR₈)(CR₉R₁₀)ₙ(Aa)ₜ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ(NR₁₁CO)(CR₉R₁₀)ₙ(OCH₂CH₂)ᵣ—, —(CR₅R₆)ₘ(OCO)(Aa)(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ(OCH₂CH₂)ᵣ—, —(CR₅R₆)ₘ—(OCO)(Aa)ₜ(CR₉R₁₀)ₙ—(OCH₂CH₂)ᵣ—, —(CR₅R₆)ₘ(OCNR₇)(Aa)ₜ(CR₉R₁₀)ₙ(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R)ₙ—, —(CR₅R₆)ₘ-thiazolyl-CO(Aa)ₜ(CCR₇R₈)ₙ—, —(CR₅R₆)ₜ-thienyl-CO(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆)tpiperazino-CO(Aa)ₜ-(CR₇R₈)ₙ—, —(CR₅R₆)ₜ—N-methylpiperazin-CO(Aa)ₜ-(CR₇R₈)ₙ—, —(CR₅R₆)ₘ—(Aa)ₜphenyl-, —(CR₅R₆)ₘ—(Aa)ₜfuryl-, —(CR₅R₆)ₘ-oxazolyl(Aa)ₜ-, —(CR₅R₆)ₘ-thiazolyl(Aa)ₜ-, —(CR₅R₆)ₘ-thienyl-(Aa)ₜ-, —(CR₅R₆)ₘ-imidazolyl(Aa)ₜ-, —(CR₅R₆)ₘ-morpholino-(Aa)ₜ-, —(CR₅R₆)ₘ-piperazino-(Aa)ₜ-, —(CR₅R₆)ₘ—N-methylpiperazino-(Aa)ₜ-, —K(CR₅R₆)ₘ(Aa)ᵣ(CR₇R₈)ₙ(OCH₂CH₂)ₜ—, —K(CR₅R₆)ₘ(CR₇R₈)ₙ(Aa)ᵣ(OCH₂CH₂)ₜ—, —K(Aa)ᵣ-(CR₅R₆)ₘ(CR₇R₈)ₙ(OCH₂CH₂)ₜ—, —K(CR₅R₆)ₘ(CR₇R₈)ₙ(OCH₂CH₂)ᵣ(Aa)ₜ-, —K(CR₅R₆)ₘ—(CR₇═CR₈)(CR₉R₁₀)ₙ(Aa)ₜ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ(Aa)ₜ(NR₁₁CO)(CR₉R₁₀)ₙ(OCH₂CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇)(Aa)ₜ(CR₉R₁₀)ₙ(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)(Aa)ₜ(CR₉R₁₀)ₙ(OCH₂CH₂)ᵣ—, —K—(CR₅R₆)ₘ(CO)(Aa)ₜ(CR₉R₁₀)ₙ(OC₁₂CH₂)ᵣ—, —K(CR₅R₆)ₘ-phenyl-CO(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—, —K(CR₅R₆)ₘ-thiazolyl-CO(Aa)ₜ-(CR₇R₈)ₙ—, —K(CR₅R₆)ₜ-thienyl-CO(CR₇R₈)ₙ—, —K(CR₅R₆)timidazolyl-CO—(CR₇R₈)ₙ—, —K(CR₅R₆)ₜmorpholino-CO(Aa)ₜ(CR₇R₈)ₙ—, —K(CR₅R₆)ₜpiperazino-CO(Aa)ₜ-(CR₇R₈)ₙ—, —K(CR₅R₆)ₜ—N-methylpiperazinCO(Aa)ₜ(CR₇R₈)ₙ—, —K(CR₅R₆)ₘ(Aa)ₜphenyl, —K—(CR₅R₆)ₘ—(Aa)ₜfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘ-piperazino-(Aa)ₜG, —K(CR₅R₆)ₘN-methylpiperazino(Aa)ₜ-; wherein m, Aa, m, n, R₃, R₄, and R₅ are described above; t and r are 0-100 independently; R₆, R₇, and R₈ are independently chosen from H; halide; C₁˜C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(═O)NH—NH—, O, S, Se, B or C₃-C₆ heteroaromatic group;
wherein the structures of the components of the linker L₁ and L₂ can be: 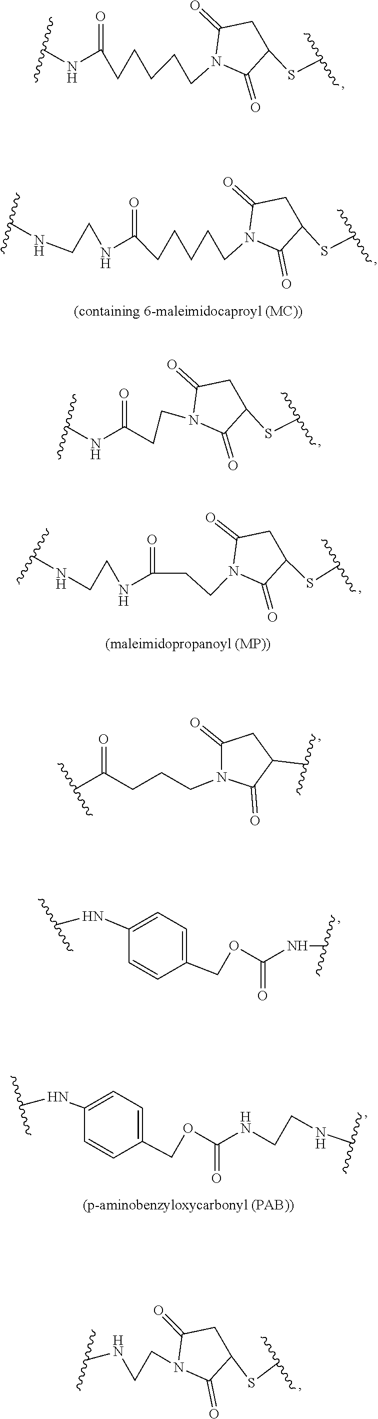 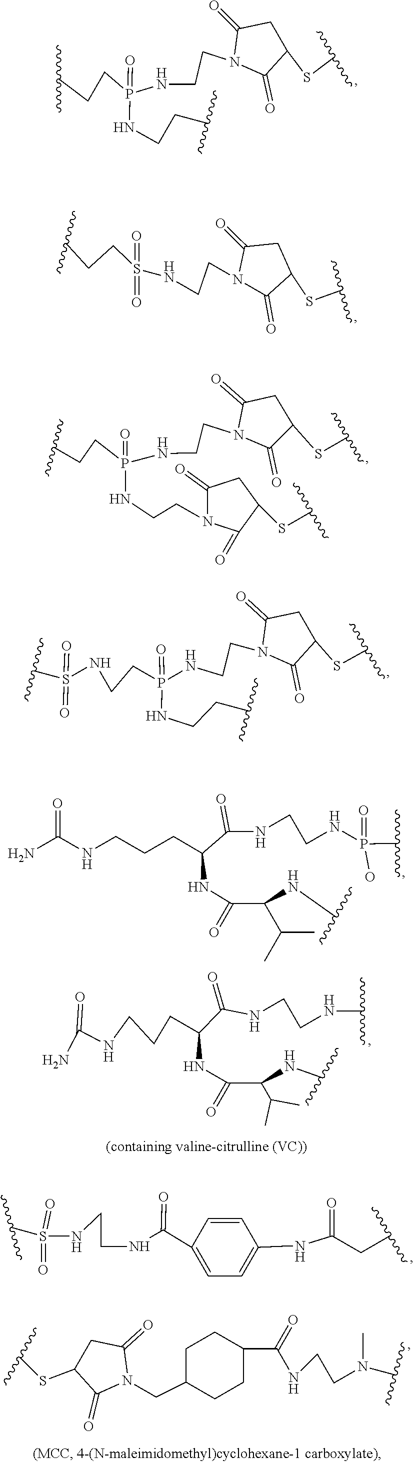 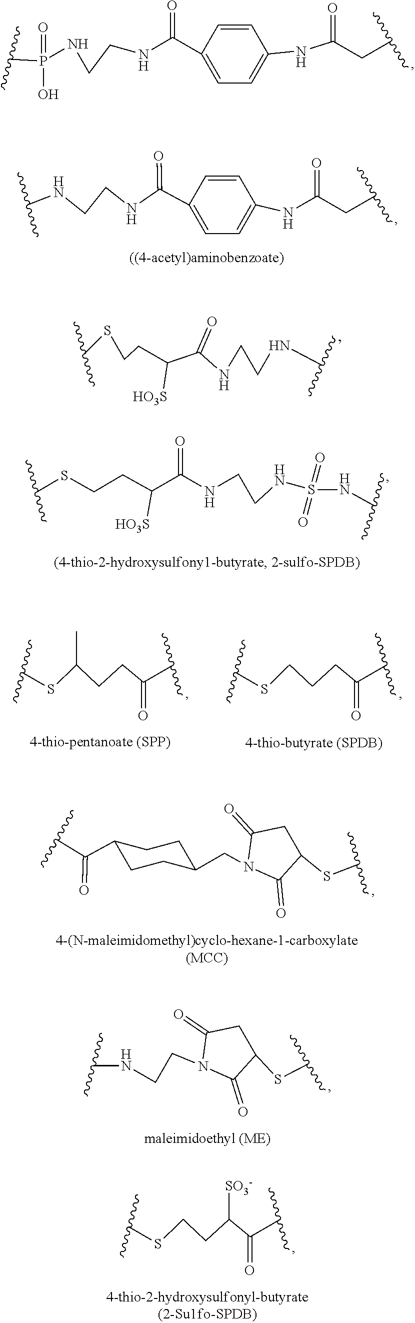 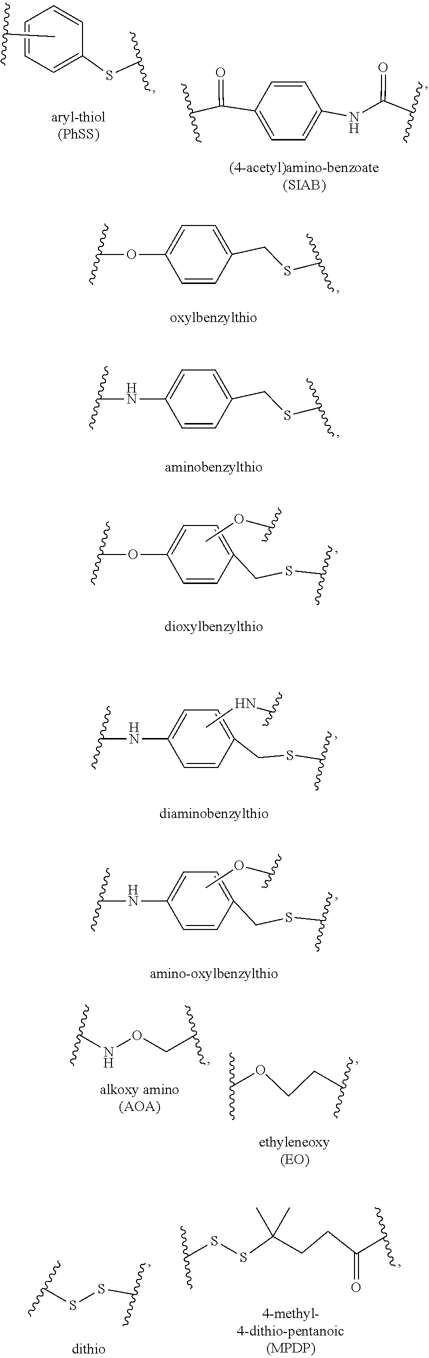 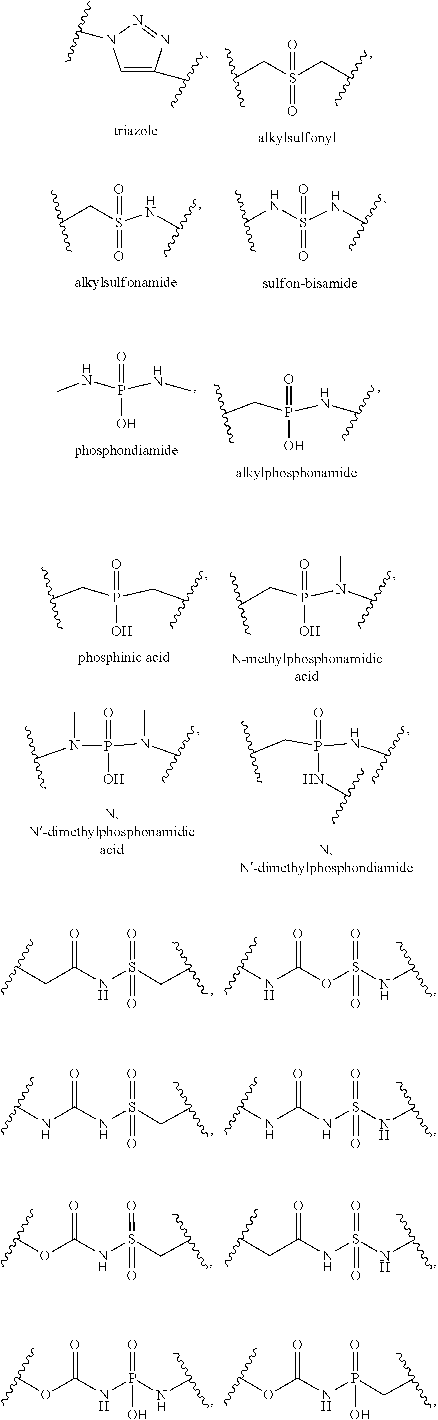 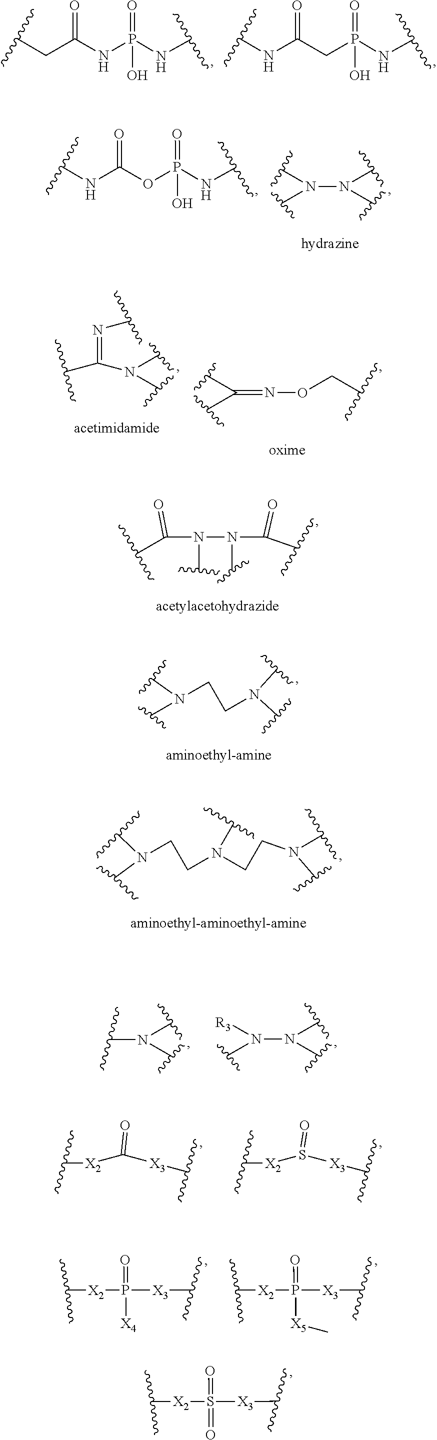 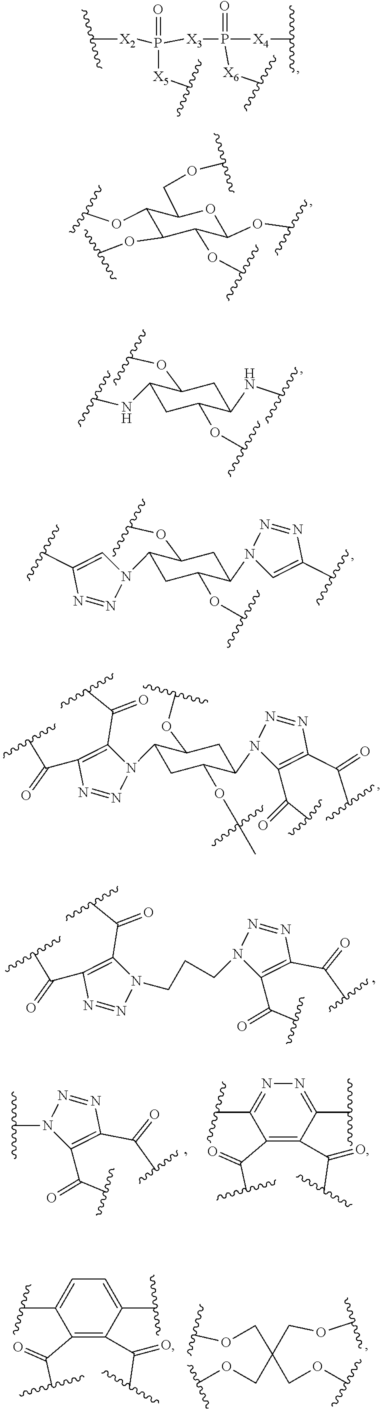 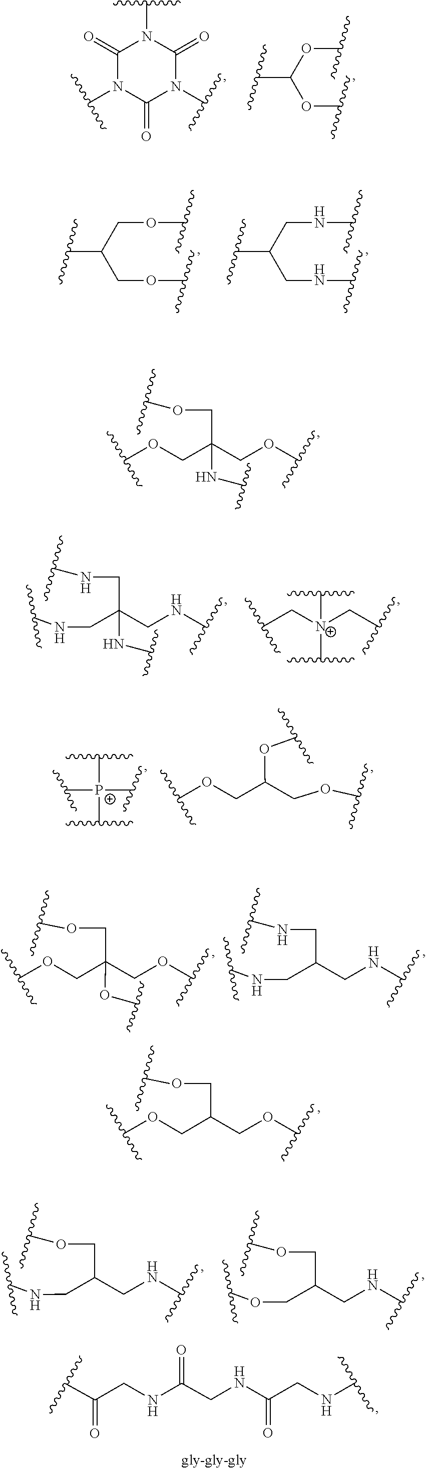 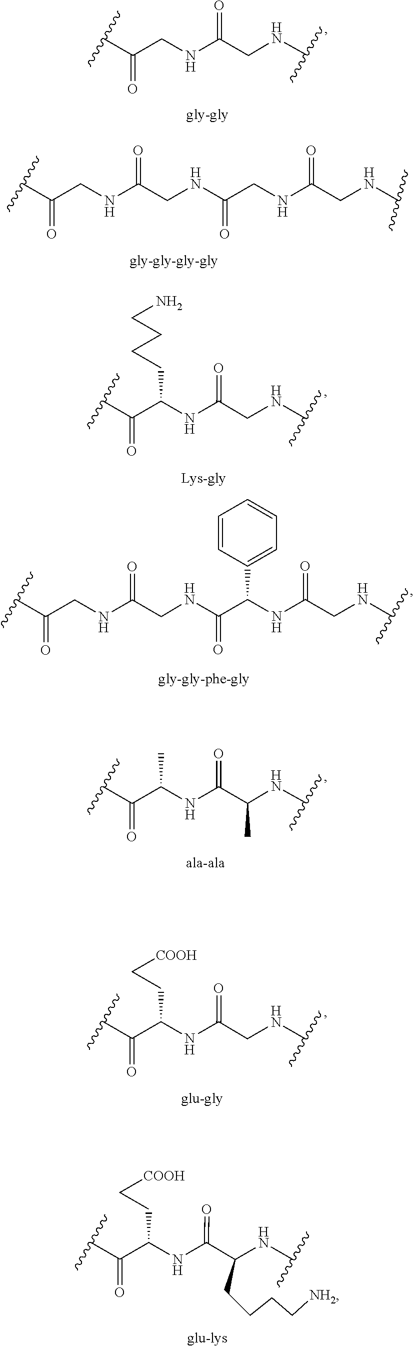 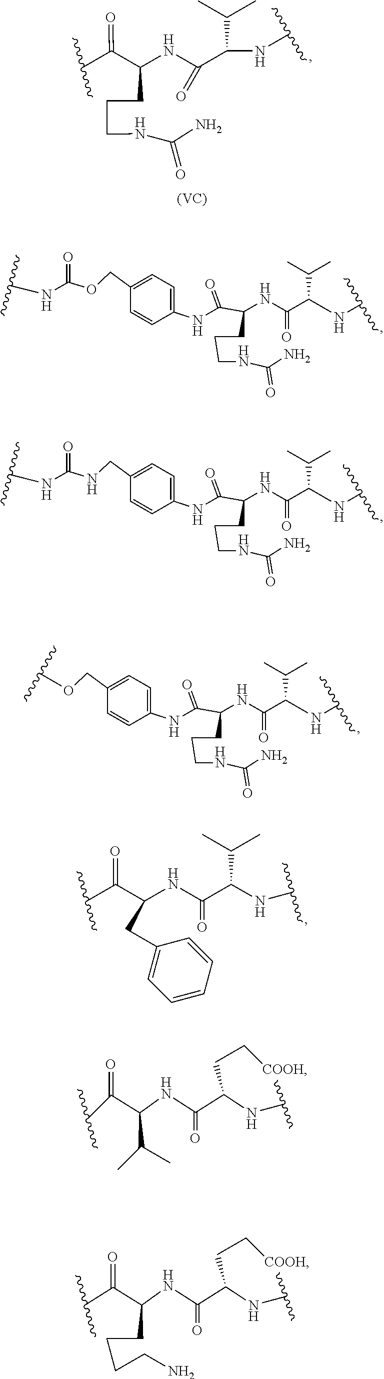 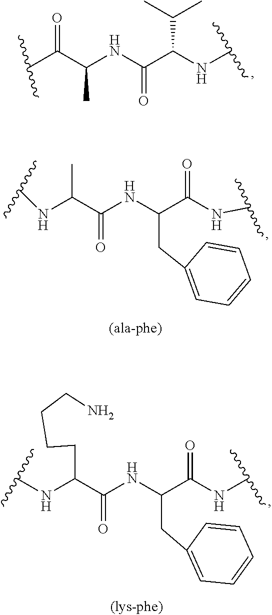 [SMILES: *N[C@H](C(=O)N[C@@H](C)C(*)=O)C(C)C ; *NC(=O)C(Cc1ccccc1)NC(=O)C(C)N* ; *NC(=O)C(Cc1ccccc1)NC(=O)C(CCCCN)N*]
or a combination of two or more of the above; wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; CH₂OR₁₂, CH₂SR₁₂, CH₂NHR₁₂, or 1-8 amino acids; wherein R₁₂ and R₁₂′ are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or polyethyleneoxy unit of formula (OCH₂CH₂)ₚ or (OCH₂CH(CH₃))ₚ, wherein p is an integer from 0 to about 1000, or a combination of two or more of the above groups;
wherein L₁, L₂, X₁, X₂, X₃, X₁′, X₂′ and X₃′ can further be independently absent;
wherein E₁ is a joint group that linked two thiol reactive groups of Lv₁ and Lv₂, and E₁ is selected from CH, CH₂, NH, NHNH, N(R₃), N(R₃)N(R₃′), N═N, N—N, P, P(═O), S, Si, C₂-C₈ alkyl, heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; a peptide containing 1-4 units of aminoacids; or one of the following structures: 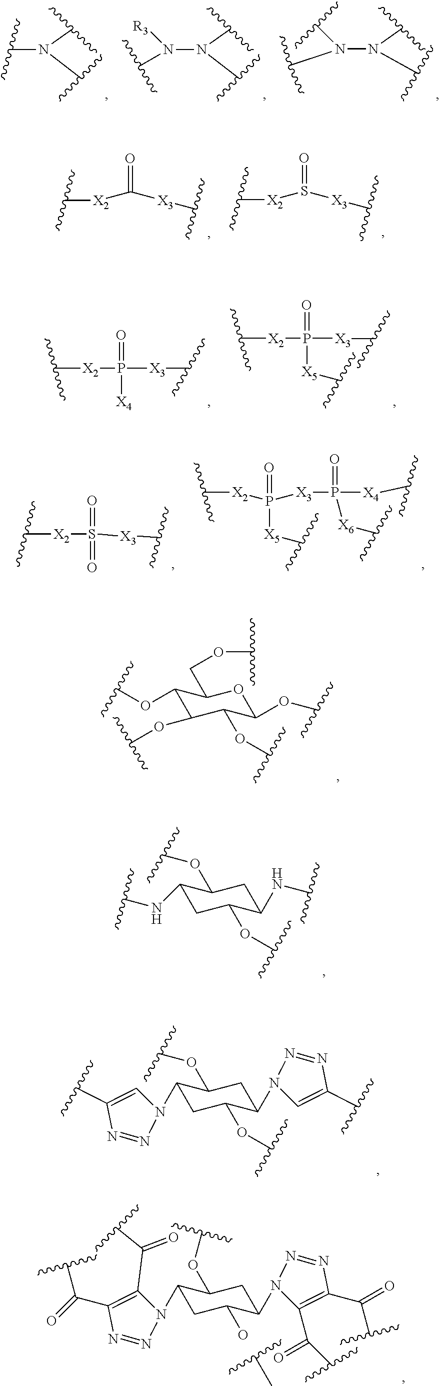 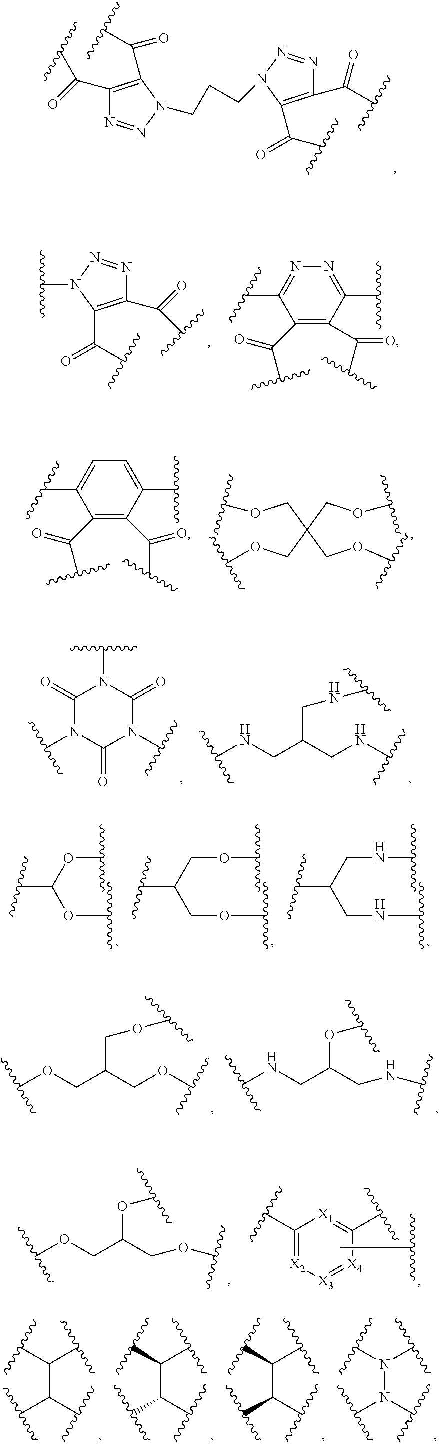 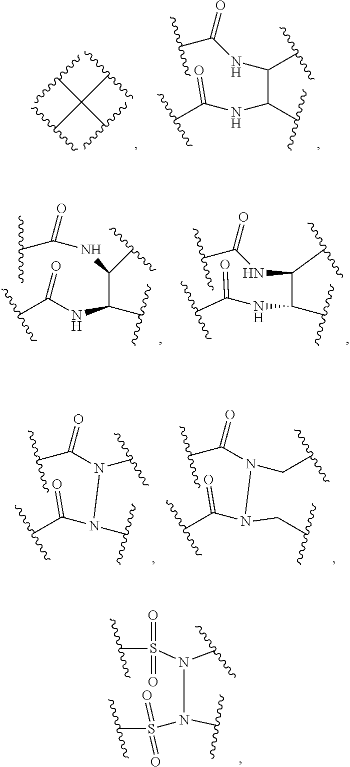
wherein [symbol] is a site of linkage;
wherein D₁ and D₂ are a cytotoxic drug, or a therapeutic drug, an immunotherapeutical short antibody-like protein, a function molecule for enhancement of binding or stabilization of the cell-binding antibody-like protein agent, a cell-surface receptor binding ligand, an antibody fragment, siRNA or DNA molecule, and are selected from:
1) Chemotherapeutic agents:
a) Alkylating agents: nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its synthetic analogues adozelesin, carzelesin and bizelesin); Duocarmycin (including the synthetic analogues, KW-2189 and CBI-TMI); Benzodiazepine dimers (including dimers of pyrrolobenzodiazepine (PBD) or tomaymycin, indolinobenzodiazepines, imidazobenzothiadiazepines, or oxazolidino-benzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines: benzodopa, carboquone, meturedopa, and uredopa; ethylenimines and methylamelamines including altretamine, triethylenemel-amine, trietylenephosphoramide, triethylenethio-phosphaoramide and trimethylolomel-amine];
b) Plant Alkaloids: Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermo-lide, bryostatins, dolostatins, auristatins, tubulysins, cephalostatins; pancratistatin; a sarcodictyin; spongistatin;
c) DNA Topoisomerase Inhibitors: Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C);
d) Anti-metabolites: [Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, frolinic acid;
e) Hormonal therapies: Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human antibody-like proteins containing a TNF domain)];
f) Kinase inhibitors: BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). antibiotics, such as the enediyne antibiotics: calicheamicins:calicheamicin γ1, δ1, α1 and β1; dynemicin: dynemicin A and deoxydynemicin; esperamicin, kedarcidin, C-1027, maduropeptin, neocarzinostatin chromophore and related chromoprotein enediyne antiobiotic chromomophores, aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
f) Polyketides (acetogenins): bullatacin and bullatacinone; gemcitabine, epoxomicins, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors: Lovastatin, Dopaminergic neurotoxins: 1-methyl-4-phenylpyridinium ion, Cell cycle inhibitors: staurosporine, Actinomycins: Actinomycin D, dactinomycin, Bleomycins: bleomycin A2, bleomycin B2, peplomycin, Anthracyclines: daunorubicin, doxorubicin (adriamycin), idarubicin, epirubicin, pirarubicin, zorubicin, mtoxantrone, MDR inhibitors: verapamil, Ca²⁺ ATPase inhibitors: thapsigargin, Histone deacetylase inhibitors: Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A; Thapsigargin, Celecoxib, glitazones, epigallocatechin gallate, Disulfiram, Salinosporamide A.; Anti-adrenals: aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine, razoxane; rhizoxin; sizofiran; spirogermanium; tenuazonic acid; triaziquone; 2, 2′, 2″-trichlorotriethylamine; trichothecenes: T-2 toxin, verrucarin A, roridin A and anguidine; urethane, siRNA, antisense drugs, and a nucleolytic enzyme;
2) An anti-autoimmune disease agent selected from the group consisting of cyclosporine, cyclosporine A, aminocaproic acid, azathioprine, bromocriptine, chlorambucil, chloroquine, cyclophosphamide, corticosteroids: amcinonide, betamethasone, budesonide, hydrocortisone, flunisolide, fluticasone propionate, fluocortolone danazol, dexamethasone, Triamcinolone acetonide, beclometasone dipropionate, DHEA, enanercept, hydroxychloroquine, infliximab, meloxicam, methotrexate, mofetil, mycophenylate, prednisone, sirolimus, and tacrolimus.
3) An anti-infectious disease agent:
a) Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b) Amphenicols:azidamfenicol, chloramphenicol, florfenicol, thiamphenicol;
c) Ansamycins: geldanamycin, herbimycin;
d) Carbapenems: biapenem, doripenem, ertapenem, imipenem/cilastatin, meropenem, panipenem;
e) Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f) Glycopeptides: bleomycin, vancomycin (oritavancin, telavancin), teicoplanin (dalbavancin), ramoplanin;
g) Glycylcyclines;
h). P-Lactamase inhibitors: penam (sulbactam, tazobactam), clavam (clavulanic acid);
i). Lincosamides: clindamycin, lincomycin;
j) Lipopeptides: daptomycin, A54145, calcium-dependent antibiotics (CDA);
k) Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l) Monobactams: aztreonam, tigemonam;
m) Oxazolidinones: linezolid;
n) Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o) Polypeptides: bacitracin, colistin, polymyxin B;
p) Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q) Streptogramins: pristinamycin, quinupristin/dalfopristin);
r) Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s) Steroid antibacterials;
t) Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines;
u) antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors: fosfomycin, nitrofurantoin, paclitaxel, platensimycin, pyrazinamide, quinupristin/dalfopristin, rifampicin (rifampin), tazobactam tinidazole, uvaricin;
4) Anti-viral drugs:
a) Entry/fusion inhibitors: aplaviroc, maraviroc, vicriviroc, gp41 (enfuvirtide), PRO 140, CD4 (ibalizumab);
b) Integrase inhibitors: raltegravir, elvitegravir, globoidnan A;
c) Maturation inhibitors: bevirimat, vivecon;
d) Neuraminidase inhibitors: oseltamivir, zanamivir, peramivir;
e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (3′-fluoro-2′, 3′-dideoxythymidine (FLT) and 3′-fluoro-2′, 3′-dideoxyguanosine (FLG), fomivirsen, ganciclovir, idoxuridine, lamivudine (3TC), 1-nucleosides (β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f) Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g) Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h) anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
5) radioisotopes (radionuclides): ³H, ¹¹C, ¹⁴C, ¹⁸F, ³²P, ³⁵, ⁶⁴Cu, ⁶⁸Ga ⁸⁶Y⁹⁹Tc, ¹¹¹In, ¹²³I, ¹²⁴I, ¹²⁵I, ¹³¹I, ¹³³Xe, ¹⁷⁷Lu, ²¹¹At, and ²¹³Bi;
6) a pharmaceutically acceptable salt, acid or derivative of any of the above drugs;
wherein Lv_{1′} and Lv_{2′} independently have one of the following structures: 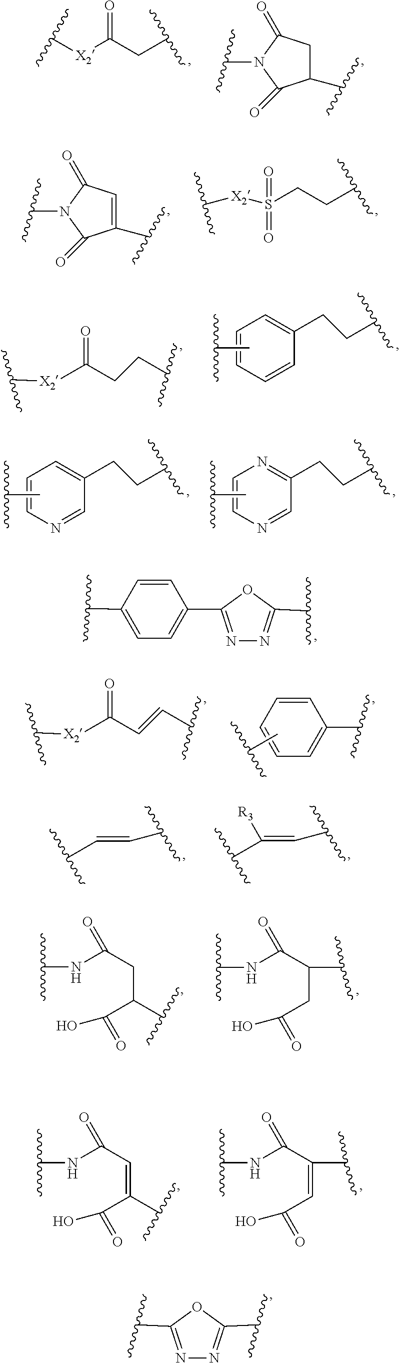
wherein X₂′ is F, Cl, Br, I, OTf, OMs, OC₆H₄(NO₂), OC₆H₃(NO₂)₂, OC₆F₅, OC₆HF₄, or Lv₃; and
R₃ is H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁;
wherein in the formula (VI) and formula (VII) the moities of, 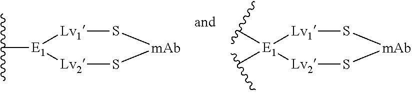
are independently selected from: 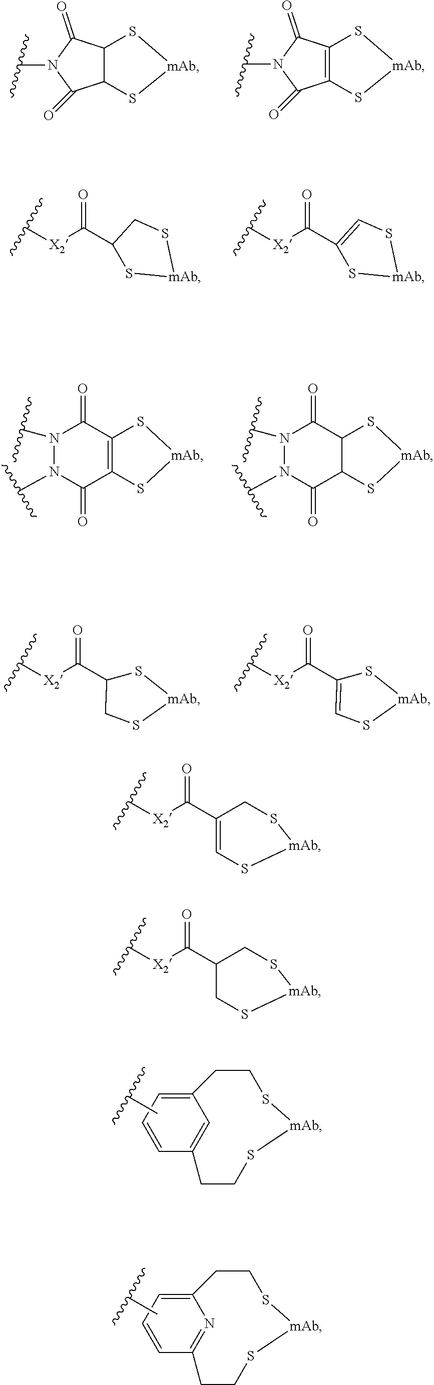 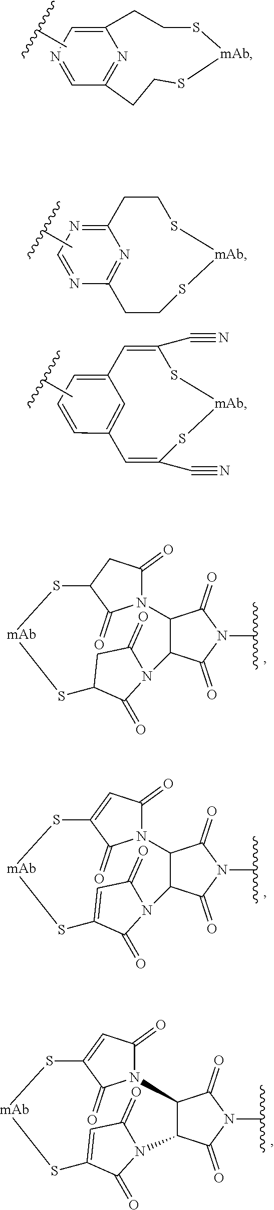 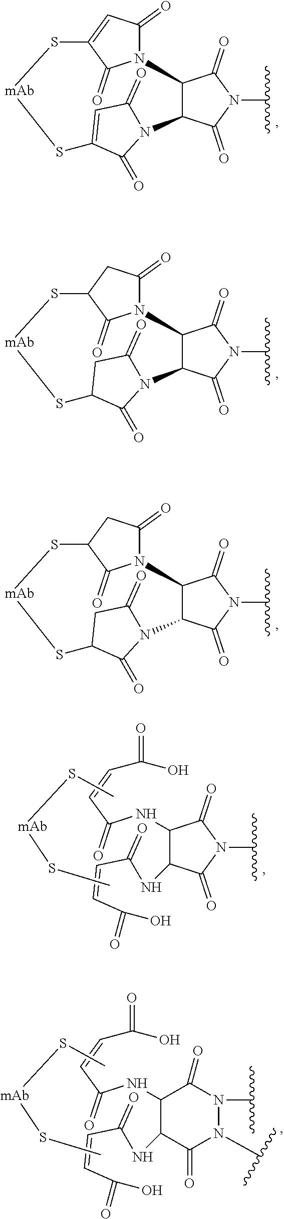 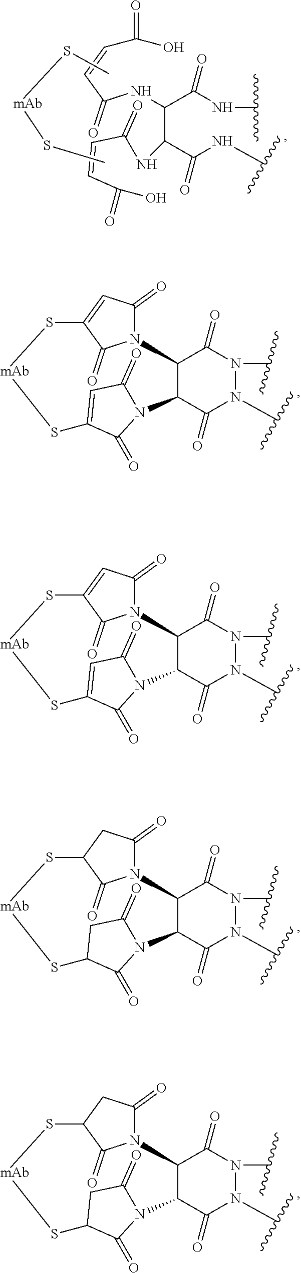 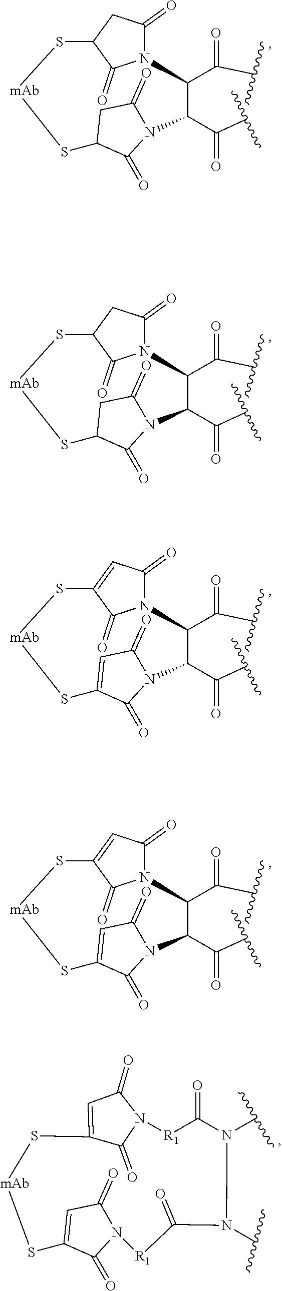 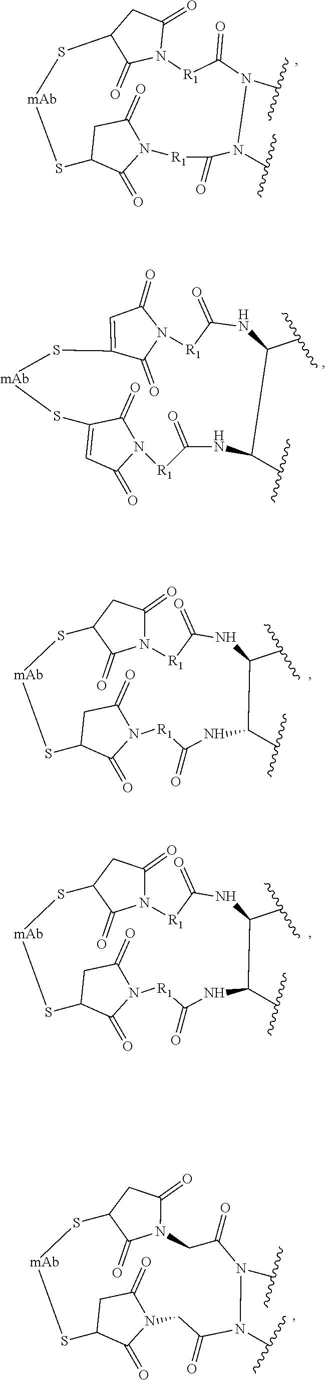 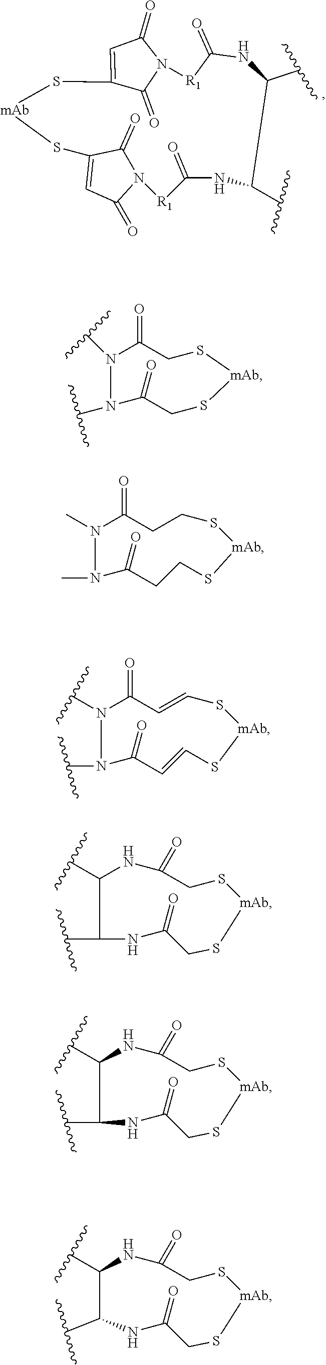 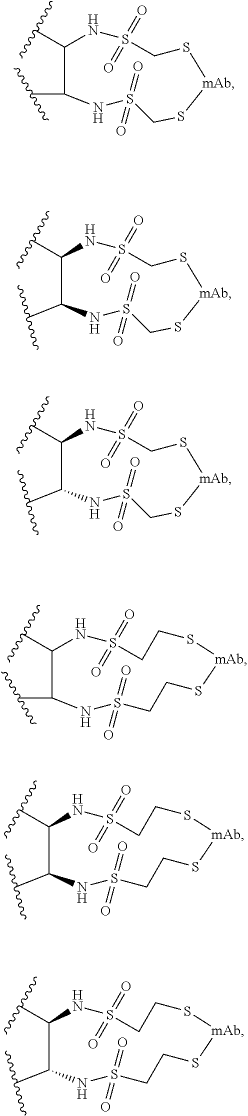 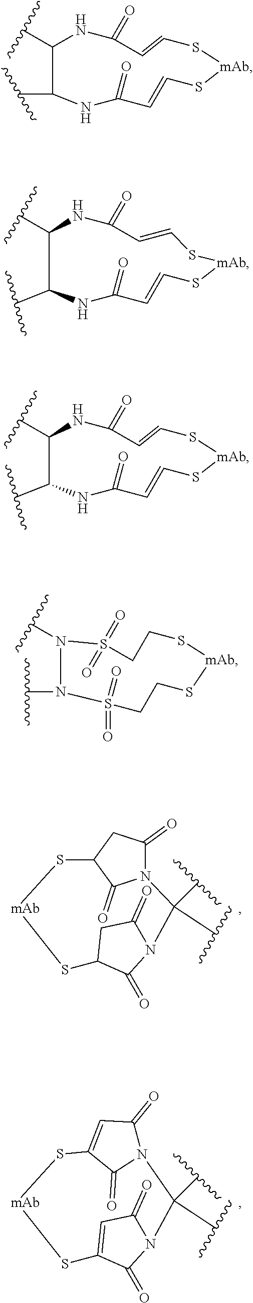 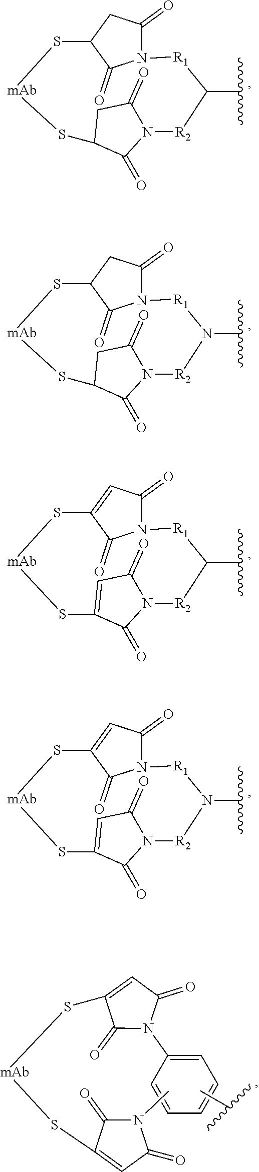 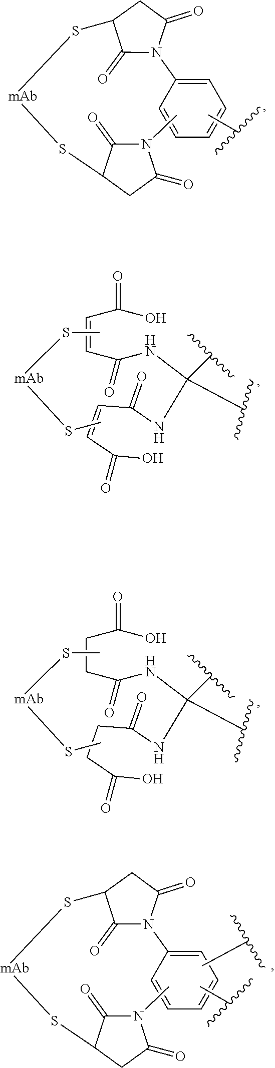 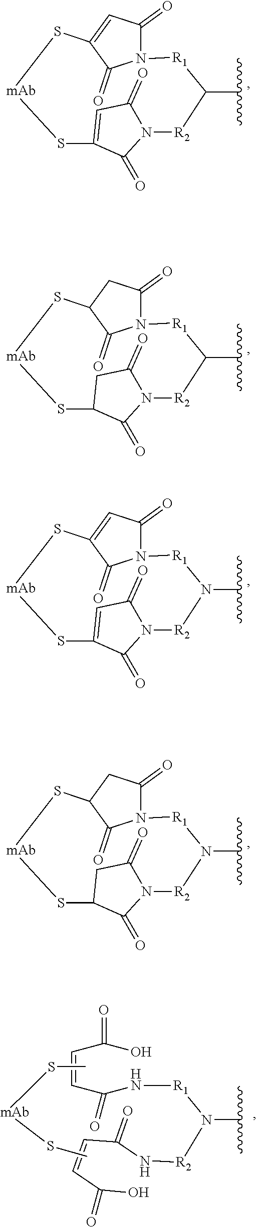 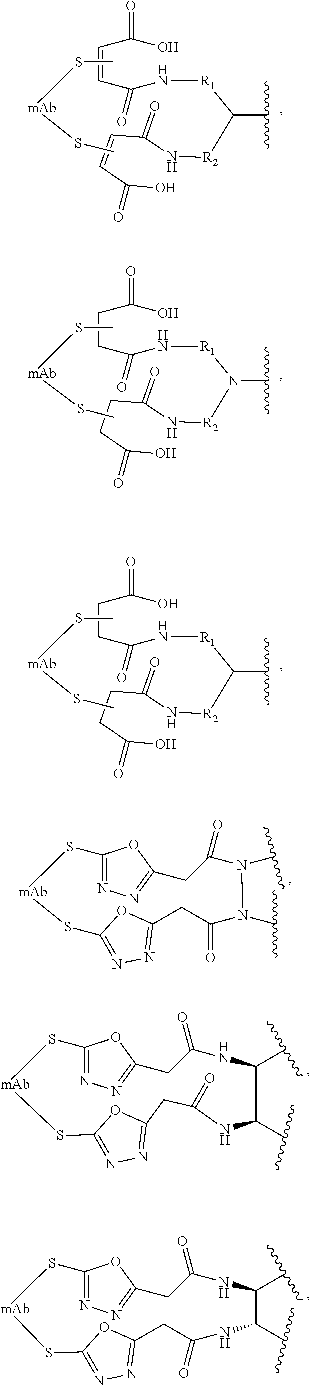
wherein R₁, R₂, X₂ and mAb are the same as defined above.

44. The conjugate of claim 43, having in vitro, in vivo or ex vivo cell killing activity.

45. The process according to claim 23, wherein a drug/antibody like protein (antibody) ratio (DAR) of the conjugate is 2-6, measured by UV at wavelength of range 240-380 nm, by hydrophobic interaction chromatography (HIC-HPLC), Capillary electrophoresis (CE), LC-MS, CE-MS, or LC-MS/MS.
